(19) United States
(12) Patent Application Publication
(10) Pub. No.: US 2004/0043382 A1
(43) Pub. Date: Mar. 4, 2004

(54) Novel proteins and nucleic acids encoding same

(76) Inventors: Muralidhara Padigaru, Branford, CT (US); Kimberly A. Spytek, New Haven, CT (US); Suresh G. Shenoy, Branford, CT (US); Raymond J. Taupier JR., East Haven, CT (US); Carol E. A. Pena, New Haven, CT (US); Li Li, Branford, CT (US); Bryan D. Zerhusen, Branford, CT (US); Vladimir Y. Gusev, Madison, CT (US); Weizhen Ji, Branford, CT (US); Linda Gorman, Branford, CT (US); Charles E. Miller, Guilford, CT (US); Ramesh Kekuda, Norwalk, CT (US); Meera Patturajan, Branford, CT (US); Esha A. Gangolli, Madison, CT (US); Corine A.M. Vernet, Branford, CT (US); Xiaojia Sasha Guo, Branford, CT (US); Velizar T. Tchernev, Branford, CT (US); Elma R. Fernandes, Branford, CT (US); Stacie J. Casman, North Haven, CT (US); Uriel M. Malyankar, Branford, CT (US); Valerie Gerlach, Branford, CT (US); Yi Liu, San Diego, CA (US); David W. Anderson, Branford, CT (US); Steven K. Spaderna, Berlin, CT (US); Elina Catterton, Madison, CT (US); Mario W. Leite, Milford, CT (US); Haihong Zhong, Guilford, CT (US); John P. Alsobrook II, Madison, CT (US); Denise M. Lepley, Branford, CT (US); Daniel K. Rieger, Branford, CT (US); Catherine E. Burgess, Wethersfield, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/092,900

(22) Filed: Mar. 7, 2002

Related U.S. Application Data
(60) Provisional application No. 60/274,322, filed on Mar. 8, 2001.
(60) Provisional application No. 60/283,675, filed on Apr. 13, 2001.
(60) Provisional application No. 60/338,092, filed on Dec. 3, 2001.
(60) Provisional application No. 60/274,281, filed on Mar. 8, 2001.
(60) Provisional application No. 60/274,191, filed on Mar. 8, 2001.
(60) Provisional application No. 60/325,681, filed on Sep. 27, 2001.
(60) Provisional application No. 60/304,354, filed on Jul. 10, 2001.
(60) Provisional application No. 60/279,995, filed on Mar. 30, 2001.
(60) Provisional application No. 60/294,899, filed on May 31, 2001.
(60) Provisional application No. 60/287,424, filed on Apr. 30, 2001.
(60) Provisional application No. 60/299,027, filed on Jun. 18, 2001.
(60) Provisional application No. 60/309,198, filed on Jul. 31, 2001.
(60) Provisional application No. 60/281,444, filed on Apr. 4, 2001.
(60) Provisional application No. 60/274,194, filed on Mar. 8, 2001.
(60) Provisional application No. 60/274,849, filed on Mar. 9, 2001.
(60) Provisional application No. 60/330,380, filed on Oct. 18, 2001.
(60) Provisional application No. 60/275,235, filed on Mar. 12, 2001.
(60) Provisional application No. 60/288,342, filed on May 3, 2001.
(60) Provisional application No. 60/275,578, filed on Mar. 13, 2001.
(60) Provisional application No. 60/291,240, filed on May 16, 2001.
(60) Provisional application No. 60/294,485, filed on May 30, 2001.
(60) Provisional application No. 60/299,310, filed on Jun. 19, 2001.
(60) Provisional application No. 60/275,579, filed on Mar. 13, 2001.
(60) Provisional application No. 60/275,601, filed on Mar. 13, 2001.
(60) Provisional application No. 60/276,000, filed on Mar. 14, 2001.
(60) Provisional application No. 60/280,900, filed on Apr. 2, 2001.
(60) Provisional application No. 60/276,776, filed on Mar. 16, 2001.
(60) Provisional application No. 60/294,889, filed on May 31, 2001.
(60) Provisional application No. 60/318,770, filed on Sep. 12, 2001.
(60) Provisional application No. 60/276,994, filed on Mar. 19, 2001.
(60) Provisional application No. 60/277,338, filed on Mar. 20, 2001.
(60) Provisional application No. 60/325,430, filed on Sep. 27, 2001.
(60) Provisional application No. 60/332,094, filed on Nov. 21, 2001.
(60) Provisional application No. 60/299,303, filed on Jun. 19, 2001.
(60) Provisional application No. 60/288,066, filed on May 2, 2001.
(60) Provisional application No. 60/277,321, filed on Mar. 20, 2001.
(60) Provisional application No. 60/280,822, filed on Apr. 2, 2001.
(60) Provisional application No. 60/277,239, filed on Mar. 20, 2001.
(60) Provisional application No. 60/277,327, filed on Mar. 20, 2001.
(60) Provisional application No. 60/277,791, filed on Mar. 21, 2001.
(60) Provisional application No. 60/333,184, filed on Nov. 14, 2001.
(60) Provisional application No. 60/277,833, filed on Mar. 22, 2001.
(60) Provisional application No. 60/318,462, filed on Sep. 10, 2001.
(60) Provisional application No. 60/288,528, filed on May 3, 2001.
(60) Provisional application No. 60/278,152, filed on Mar. 23, 2001.
(60) Provisional application No. 60/332,272, filed on Nov. 14, 2001.
(60) Provisional application No. 60/278,894, filed on Mar. 26, 2001.
(60) Provisional application No. 60/312,903, filed on Aug. 16, 2001.
(60) Provisional application No. 60/333,272, filed on Nov. 14, 2001.
(60) Provisional application No. 60/279,036, filed on Mar. 27, 2001.
(60) Provisional application No. 60/332,172, filed on Nov. 14, 2001.
(60) Provisional application No. 60/337,426, filed on Dec. 3, 2001.
(60) Provisional application No. 60/278,999, filed on Mar. 27, 2001.
(60) Provisional application No. 60/279,344, filed on Mar. 28, 2001.
(60) Provisional application No. 60/332,271, filed on Nov. 14, 2001.
(60) Provisional application No. 60/291,099, filed on May 16, 2001.
(60) Provisional application No. 60/291,190, filed on May 15, 2001.
(60) Provisional application No. 60/280,233, filed on Mar. 30, 2001.
(60) Provisional application No. 60/280,802, filed on Apr. 2, 2001.
(60) Provisional application No. 60/335,301, filed on Oct. 31, 2001.
(60) Provisional application No. 60/337,185, filed on Dec. 4, 2001.
(60) Provisional application No. 60/345,705, filed on Jan. 3, 2002.

(51) Int. Cl. C12Q 1/68; C07H 21/04; C12N 9/00; C12P 21/02; C12N 5/06; C07K 14/47

(52) U.S. Cl. 435/6; 435/69.1; 435/183; 435/320.1; 435/325; 530/350; 536/23.2

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases.

### RELATED APPLICATIONS

This is a request for filing a new nonprovisional application under 37 C.F.R. §1.53(b). This application claims priority to U.S. S. No. 60/274,322 filed on Mar. 8, 2001 (Cura 590); U.S. S. No. 60/283,675 filed on Apr. 13, 2001 (Cura 590D1); U.S. S. No. 60/338,092 filed on Dec. 3, 2001 (Cura 590D2); U.S. S. No. 60/274,281 filed on Mar. 8, 2001 (Cura 591); U.S. S. No. 60/274,101 filed on Mar. 8, 2001 (Cura 592); U.S. S. No. 60/325,681 filed on Sep. 27, 2001 (Cura 592J1); U.S. S. No. 60/304,354 filed on Jul. 10, 2001 (Cura 592I1); U.S. S. No. 60/279,995 filed on Mar. 30, 2001 (Cura 592H1); U.S. S. No. 60/294,899 filed on May 31, 2001 (Cura 592E1); U.S. S. No. 60/287,424 filed on Apr. 30, 2001 (Cura 592D1); U.S. S. No. 60/299,027 filed on Jun. 18, 2001 (Cura 592D2); U.S. S. No. 60/309,198 filed on Jul. 31, 2001 (Cura 592C1); U.S. S. No. 60/281,194 filed on Apr. 4, 2001 (Cura 592A1); U.S. S. No. 60/274,194 filed on Mar. 8, 2001 (Cura 593); U.S. S. No. 60/274,849 filed on Mar. 9, 2001 (Cura 594); U.S. S. No. 60/330,380 filed on Oct. 18, 2001 (Cura 594C1); U.S. S. No. 60/275,235 filed on Mar. 12, 2001 (Cura 595); U.S. S. No. 60/288,342 filed on May 3, 2001 (Cura 595J1); U.S. S. No. 60/275,578 filed on Mar. 13, 2001 (Cura 596); U.S. S. No. 60/291,240 filed on May 16, 2001 (Cura 596I1); U.S. S. No. 60/294,485 filed on May 30, 2001 (Cura 596B1); U.S. S. No. 60/299,310 filed on Jun. 19, 2001 (Cura 596A1); U.S. S. No. 60/275,579 filed on Mar. 13, 2001 (Cura 597); U.S. S. No. 60/275,601 filed on Mar. 13, 2001 (Cura 598); U.S. S. No. 60/276,000 filed on Mar. 14, 2001 (Cura 599); U.S. S. No. 60/280,900 filed on Apr. 2, 2001 (Cura 599E1); U.S. S. No. 60/276,776 filed on Mar. 16, 2001 (Cura 600); U.S. S. No. 60/294,889 filed on May 31, 2001 (Cura 600G 1); U.S. S. No. 60/318,770 filed on Sep. 12, 2001 (Cura 600E1); U.S. S. No. 60/276,994 filed on Mar. 19, 2001 (Cura 604); U.S. S. No. 60/277,338 filed on Mar. 20, 2001 (Cura 607); U.S. S. No. 60/325,430 filed on Sep. 27, 2001 (Cura 607J1); U.S. S. No. 60/332,094 filed on Nov. 21, 2001 (Cura 607C1); U.S. S. No. 60/299,303 filed on Jun. 19, 2001 (Cura 607B1); U.S. S. No. 60/288,066 filed on May 2, 2001 (Cura 607A1); U.S. S. No. 60/277,321 filed on Mar. 20, 2001 (Cura 608); U.S. S. No. 60/280,822 filed on Apr. 2, 2001 (Cura 608A); U.S. S. No. 60/277,239 filed on Mar. 20, 2001 (Cura 609); U.S. S. No. 60/277,327 filed on Mar. 20, 2001 (Cura 610); U.S. S. No. 60/277,791 filed on Mar. 21, 2001 (Cura 611); U.S. S. No. 60/333,184 filed on Nov. 14, 2001 (Cura 611H1); U.S. S. No. 60/277,833 filed on Mar. 22, 2001 (Cura 612); U.S. S. No. 60/318,462 filed on Sep. 10, 2001 (Cura 612J1); U.S. S. No. 60/288,528 filed on May 3, 2001 (Cura 612A1); U.S. S. No. 60/278,152 filed on Mar. 23, 2001 (Cura 613); U.S. S. No. 60/332,272 filed on Nov. 14, 2001 (Cura 613D1); U.S. S. No. 60/278,894 filed on Mar. 26, 2001 (Cura 614); U.S. S. No. 60/312,903 filed on Aug. 16, 2001 (Cura 614C1); U.S. S. No. 60/333,272 filed on Nov. 14, 2001 (Cura 614C2); U.S. S. No. 60/279,036 filed on Mar. 27, 2001 (Cura 615); U.S. S. No. 60/332,172 filed on Nov. 14, 2001 (Cura 615I1); U.S. S. No. 60/337,426 filed on Dec. 3, 2001 (Cura 615I2); U.S. S. No. 60/278,999 filed on Mar. 27, 2001 (Cura 616); U.S. S. No. 60/279,344 filed on Mar. 28, 2001 (Cura 617); U.S. S. No. 60/332,271 filed on Nov. 14, 2001 (Cura 617J1); U.S. S. No. 60/291,099 filed on May 16, 2001 (Cura 617H1); U.S. S. No. 60/291,190 filed on May 15, 2001 (Cura 617E1); U.S. S. No. 60/280,233 filed on Mar. 30, 2001 (Cura 618); U.S. S. No. 60/280,802 filed on Apr. 2, 2001 (Cura 621); U.S. S. No. 60/335,301 filed on Oct. 31, 2001 (Cura 621 F1); U.S. S. No. 60/337,185 filed on Dec. 4, 2001 (Cura 621D1); and U.S. S. No. 60/345,705 filed on Jan. 3, 2002 (Cura 621B1).

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions. The present invention discloses novel associations of proteins and polypeptides and the nucleic acids that encode them with various diseases or pathologies. The proteins and related proteins that are similar to them, are encoded by a cDNA and/or by genomic DNA. The proteins, polypeptides and their cognate nucleic acids were identified by Curagen Corporation in certain cases. The XYZase-encoded protein and any variants, thereof, are suitable as diagnostic markers, targets for an antibody therapeutic and targets for small molecule drugs. As such the current invention embodies the use of recombinantly expressed and/or endogenously expressed protein in various screens to identify such therapeutic antibodies and/or therapeutic small molecules.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways are constituted of extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion protein effectors. In a clinical setting a subject may be suspected of suffering from a condition brought on by diminished or suppressed levels of a protein effector of interest. Therefore there is a need to be able to assay for the level of the protein effector of interest in a biological sample from such a subject, and to compare the level with that characteristic of a nonpathological condition. There further is a need to provide the protein effector as a product of manufacture. Administration of the effector to a subject in need thereof is useful in treatment of the pathological condition, or the protein effector deficiency or suppression may be favorably acted upon by the administration of another small molecule drug product. Accordingly, there is a need for a method of treatment of a pathological condition brought on by a diminished or suppressed levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

It is an objective of this invention to provide at least one target biopolymer that is intended to serve as the macromolecular component in a screening assay for identifying candidate pharmaceutical agents.

It is another objective of the present invention to provide screening assays that positively identify candidate pharmaceutical agents from among a combinatorial library of low molecular weight substances or compounds.

It is still a further objective of this invention to employ the candidate pharmaceutical agents in any of a variety of in vitro, ex vivo and in vivo assays in order to identify pharmaceutical agents with advantageous therapeutic applications in the treatment of a disease, pathology, or abnormal state or condition in a mammal.

### SUMMARY OF THE INVENTION

The invention is based in part upon the discovery of nucleic acid sequences encoding novel polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 178, or polypeptide sequences, which represents the group consisting of SEQ ID NO: 2n, wherein n is an integer between 1 and 178.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 178, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table 1 provides a summary of the NOVX nucleic acids and their encoded polypeptides.

TABLE 1
Sequences and Corresponding SEQ ID Numbers

| | | Nucleic | Amino | |
|---|---|---|---|---|
| | | Acid | Acid | |
| | | SEQ | SEQ | |
| NOVX | Internal | ID | ID | |
| No. | Acc. No. | NO. | NO. | Homology |
| 1a | CG58522-01 | 1 | 2 | human platelet |
| | | | | activating factor |
| | | | | acetylhydrolase |
| 2a | CG58520-01 | 3 | 4 | GABA(A) receptor |
| 2b | CG58520-02 | 5 | 6 | GABA(A) receptor |
| 2c | CG58520-03 | 7 | 8 | GABA(A) receptor |
| 3a | CG58518-01 | 9 | 10 | GABA(A) receptor |
| 4a | CG58516-01 | 11 | 12 | Beta transducin |
| 5a | CG58473-01 | 13 | 14 | Protein kinase |
| 6a | CG58470-01 | 15 | 16 | UDP-N- |
| | | | | acetylhexosamine |
| | | | | pyrophosphorylase |
| 7a | CG58593-01 | 17 | 18 | ubiquitin 52 like |
| 8a | CG57871-01 | 19 | 20 | tousled like kinase like |
| 9a | CG58590-01 | 21 | 22 | guanylate kinase like |
| 9b | CG58590-02 | 23 | 24 | guanylate kinase like |
| 10a | CG58572-01 | 25 | 26 | glucosamine phosphate |
| | | | | N acetyltransferase like |
| 10b | CG58572-02 | 27 | 28 | glucosamine phosphate |
| | | | | N acetyltransferase like |
| 11a | CG58564-01 | 29 | 30 | Protein tyrosine |
| | | | | phosphatase like |
| 11b | CG58564-02 | 31 | 32 | Protein tyrosine |
| | | | | phosphatase like |
| 11c | CG58564-03 | 33 | 34 | Dual-Specificity |
| | | | | phosphatase like |
| 11d | CG58564-04 | 35 | 36 | Dual-Specificity |
| | | | | phosphatase like |
| 12a | CG57819-01 | 37 | 38 | RPGR interacting |
| | | | | protein 1 like |
| 13a | CG57789-01 | 39 | 40 | RAS like protein |
| | | | | RRP22 like |
| 13b | CG57789-02 | 41 | 42 | RAS like protein |
| | | | | RRP22 like |
| 14a | CG57758-01 | 43 | 44 | sodium/lithium |
| | | | | dependent |
| | | | | dicarboxylate |
| | | | | transporter like |
| 14b | CG57758-02 | 45 | 46 | sodium/lithium |
| | | | | dependent |
| | | | | dicarboxylate |
| | | | | transporter like |
| 14c | CG57758-03 | 47 | 48 | sodium/lithium |
| | | | | dependent |
| | | | | dicarboxylate |
| | | | | transporter like |
| 14d | CG57758-04 | 49 | 50 | sodium/lithium |
| | | | | dependent |
| | | | | dicarboxylate |
| | | | | transporter like |
| 14e | CG57758-05 | 51 | 52 | sodium/lithium |
| | | | | dependent |
| | | | | dicarboxylate |
| | | | | transporter like |
| 15a | CG57732-01 | 53 | 54 | Ca 2 + calmodulin |
| | | | | dependent protein |
| | | | | kinase IV kinase like |
| 15b | CG57732-02 | 55 | 56 | Ca 2 + calmodulin |
| | | | | dependent protein |
| | | | | kinase IV kinase like |
| 15c | CG57732-03 | 57 | 58 | Ca 2 + calmodulin |
| | | | | dependent protein |
| | | | | kinase IV kinase like |
| 16a | CG57709-01 | 59 | 60 | TCE2 like |
| 17a | CG57700-01 | 61 | 62 | hydoxyacylglutathione |
| | | | | hydrolase like |
| 17b | CG57700-02 | 63 | 64 | hydoxyacylglutathione |
| | | | | hydrolase like |
| 17c | CG57700-03 | 65 | 66 | hydoxyacylglutathione |
| | | | | hydrolase like |
| 17d | CG57700-04 | 67 | 68 | hydoxyacylglutathione |
| | | | | hydrolase like |
| 18a | CG58553-01 | 69 | 70 | vasopressin receptor |
| | | | | like |
| 19a | CG58626-01 | 71 | 72 | phosphatidic acid |
| | | | | preferring |
| | | | | phospholipase A1 like |
| 20a | CG57597-01 | 73 | 74 | hypothetical protein |
| | | | | like |
| 21a | CG57804-01 | 75 | 76 | Talin like |
| 22a | CG57551-01 | 77 | 78 | NAC-1 like |
| 23a | CG57411-01 | 79 | 80 | Kelch like |
| 24a | CG57399-01 | 81 | 82 | phospholipase |
| | | | | ADRAB-B precursor |
| | | | | like |
| 24b | CG57399-02 | 83 | 84 | phospholipase |
| | | | | ADRAB-B precursor |
| | | | | like |
| 24c | CG57399-03 | 85 | 86 | phospholipase |
| | | | | ADRAB-B precursor |
| | | | | like |
| 25a | CG59311-01 | 87 | 88 | acyl-coenzyme A |
| | | | | thioester hydrolase |
| 25b | CG59311-02 | 89 | 90 | peroxisomal acyl- |
| | | | | coenzyme A thioeseter |
| | | | | hydrolase like |
| 25c | CG59311-03 | 91 | 92 | peroxisomal acyl- |
| | | | | coenzyme A thioeseter |
| | | | | hydrolase like |
| 26a | CG59309-01 | 93 | 94 | acyl-coenzyme A |
| | | | | thioester hydrolase |
| 27a | CG57364-01 | 95 | 96 | CG6896 |
| 28a | CG59348-01 | 97 | 98 | cytoplasmic protein |
| | | | | (patent calls this Cyclin |
| | | | | L-like) |
| 29a | CG59245-01 | 99 | 100 | glucose 6-phosphatase |
| 29b | CG59245-02 | 101 | 102 | glucose 6-phosphatase |
| 30a | CG59241-01 | 103 | 104 | Amiloride-sensitive |
| | | | | sodium channel |
| 31a | CG58602-01 | 105 | 106 | FAD binding domain |
| | | | | containing protein |
| 32a | CG58468-01 | 107 | 108 | Serum Amyloid Protein |
| 33a | CG58183-01 | 109 | 110 | N-Methyl-D-Aspartate |
| | | | | receptor |
| 34a | CG59315-01 | 111 | 112 | Connexin |
| 35a | CG59203-01 | 113 | 114 | lysozyme C |
| 35b | CG59203-02 | 115 | 116 | lysozyme C |
| 36a | CG58662-01 | 117 | 118 | cytoplasmic protein |
| 36b | CG58662-02 | 119 | 120 | cytoplasmic protein |
| 37a | CG58584-01 | 121 | 122 | 405 ribosomal protein |
| | | | | S29 like |
| 38a | CG58538-01 | 123 | 124 | Histone deacetylase |
| | | | | complex protein 66 like |
| 39a | CG59371-01 | 125 | 126 | expressed cytoplasmic |
| | | | | protein like |
| 40a | CG59346-01 | 127 | 128 | cortactin binding |
| | | | | protein 1 like |
| 41a | CG57814-01 | 129 | 130 | Basic I 19 like homo |
| | | | | sapiens |
| 41b | CG57814-02 | 131 | 132 | Basic I 19 like homo |
| | | | | sapiens |
| 42a | CG59327-01 | 133 | 134 | Monocarboxylate |
| | | | | transporter 1 like |
| 43a | CG59494-01 | 135 | 136 | myelin P2 like |
| 44a | CG59432-0l | 137 | 138 | chloride channel like |
| 44b | CG59432-02 | 139 | 140 | chloride channel like |
| 45a | CG59394-01 | 141 | 142 | GPCR like |
| 46a | CG59383-01 | 143 | 144 | D6MM5E PROTEIN |
| | | | | like |
| 46b | CG59383-02 | 145 | 146 | D6MM5E PROTEIN |
| | | | | like |
| 47a | CG58526-01 | 147 | 148 | scramblase like |
| 48a | CG57851-01 | 149 | 150 | sulfotransferase like |
| 49a | CG59377-01 | 151 | 152 | epsin like |
| 50a | CG59258-01 | 153 | 154 | transcriptional activator |
| | | | | like |
| 51a | CG59492-01 | 155 | 156 | Myosin Head (Motor |
| | | | | Domain) like |
| 52a | CG59564-01 | 157 | 158 | Sorting nexin 6 like |
| 53a | CG59553-01 | 159 | 160 | Secretory protein SECS |
| | | | | like |
| 54a | CG59545-01 | 161 | 162 | Placental protein 13 |
| | | | | like |
| 55a | CG59435-01 | 163 | 164 | Nedd-1 like |
| 55b | CG59435-02 | 165 | 166 | Nedd-1 like |
| 56a | CG59439-01 | 167 | 168 | Xenobiotic/medium- |
| | | | | chain fatty acid: CoA |
| | | | | ligase form XL-III like |
| 56b | CG59439-02 | 169 | 170 | Xenobiotic/medium- |
| | | | | chain fatty acid: CoA |
| | | | | ligase form XL-III like |
| 57a | CG59354-01 | 171 | 172 | phosducin like |
| 57b | CG59354-02 | 173 | 174 | phosducin like |
| 57c | CG59354-03 | 175 | 176 | phosducin like |
| 58a | CG59319-01 | 177 | 178 | phosducin like |
| 58b | CG59319-02 | 179 | 180 | phosducin like |
| 59a | CG59576-01 | 181 | 182 | GPCR like |
| 60a | CG59557-01 | 183 | 184 | GPCR like |
| 61a | CG59555-01 | 185 | 186 | GPCR like |
| 62a | CG59551-01 | 187 | 188 | GPCR like |
| 63a | CG59540-01 | 189 | 190 | GPCR like |
| 64a | CG59280-01 | 191 | 192 | GPCR like |
| 64b | CG59280-02 | 193 | 194 | GPCR like |
| 65a | CG59568-01 | 195 | 196 | GPCR like |
| 66a | CG59224-01 | 197 | 198 | GPCR like |
| 67a | CG59222-01 | 199 | 200 | GPCR like |
| 68a | CG59220-01 | 201 | 202 | GPCR like |
| 69a | CG59218-0l | 203 | 204 | GPCR like |
| 70a | CG59216-01 | 205 | 206 | GPCR like |
| 71a | CG59214-01 | 207 | 208 | GPCR like |
| 72a | CG59211-01 | 209 | 210 | GPCR like |
| 73a | CG59276-01 | 211 | 212 | Dihydroorotate |
| | | | | dehydrogenase like |
| 74a | CG59268-01 | 213 | 214 | monooxygenase like |
| 75a | CG59549-01 | 215 | 216 | H326 like (cytoplasmic |
| | | | | protein with WD repeat |
| | | | | domain) |
| 76a | CG59641-01 | 217 | 218 | Acetyl-CoA |
| | | | | Carboxylase 2 like |
| 77a | CG59630-01 | 219 | 220 | Midnolin like |
| 78a | CG59561-01 | 221 | 222 | ACYL COENZYME A |
| | | | | THIOESTER |
| | | | | HYDROLASE like |
| 79a | CG59452-01 | 223 | 224 | CELL |
| | | | | PROLIFERATION |
| | | | | RELATED PROTEIN |
| | | | | CAP like |
| 80a | CG59572-01 | 225 | 226 | Pseudouridine Synthase |
| | | | | 3 like |
| 80b | CG59572-02 | 227 | 228 | Pseudouridine Synthase |
| | | | | 3 like |
| 81a | CG59522-01 | 229 | 230 | Myosin like |
| 82a | CG59520-01 | 231 | 232 | Farnesyl- |
| | | | | pyrophosphate |
| | | | | synthetase like |
| 83a | CG59758-01 | 233 | 234 | UBIQUITIN like |
| 83b | CG59758-02 | 235 | 236 | UBIQUITIN like |
| 84a | CG59586-01 | 237 | 238 | glucokinase like |
| 85a | CG59704-01 | 239 | 240 | serine/threonine kinase |
| | | | | like |
| 86a | CG59628-01 | 241 | 242 | Short-chain |
| | | | | dehydrogenase like |
| 87a | CG59516-01 | 243 | 244 | Calponin like |
| 87b | CG59516-02 | 245 | 246 | Calponin like |
| 88a | CG59671-02 | 247 | 248 | acyl-coenzyme A |
| | | | | thioester hydrolase |
| 89a | CG56870-01 | 249 | 250 | NDRG3 like |
| 89b | CG56870-02 | 251 | 252 | NDRG3 like |
| 89c | CG56870-03 | 253 | 254 | NDRG3 like |
| 89d | CG56870-04 | 255 | 256 | NDRG3 like |
| 89e | CG56870-05 | 257 | 258 | NDRG3 like |
| 90a | CG59764-01 | 259 | 260 | Ferritin like |
| 91a | CG59710-01 | 261 | 262 | P14 like |
| 92a | CG59754-02 | 263 | 264 | Downs syndrome cell |
| | | | | adhesion molecule like |
| 92b | CG59754-01 | 265 | 266 | Downs syndrome cell |
| | | | | adhesion molecule like |
| 93a | CG59800-01 | 267 | 268 | HEPARAN SULFATE |
| | | | | D-GLUCOSAMINYL |
| | | | | 3-O- |
| | | | | SULFOTRANSFERASE-3B |
| | | | | like |
| 94a | CG59761-01 | 269 | 270 | AXIN I (AXIS |
| | | | | INHIBITION |
| | | | | PROTEIN I) (HAXIN) |
| | | | | like |
| 95a | CG59756-01 | 271 | 272 | JUNCTOPHILIN |
| | | | | TYPE 2 like |
| 96a | CG59708-01 | 273 | 274 | Ubiquitin carboxyl- |
| | | | | terminal hydrolase 21 |
| | | | | like |
| 96b | CG59708-02 | 275 | 276 | Ubiquitin carboxyl- |
| | | | | terminal hydrolase 21 |
| | | | | like |
| 96c | CG59708-03 | 277 | 278 | Ubiquitin carboxyl- |
| | | | | terminal hydrolase 21 |
| | | | | like |
| 97a | CG59559-01 | 279 | 280 | BA12M19.1.3 like |
| 98a | CG59669-01 | 281 | 282 | carbonyl reductase |
| | | | | (called NADPH- |
| | | | | dependent carbonyl |
| | | | | reductase-like in |
| | | | | patent) |
| 99a | CG58624-01 | 283 | 284 | metal transporter |
| 100a | CG59679-01 | 285 | 286 | carbonyl reductase |
| 101a | CG59644-01 | 287 | 288 | CG12091 (putative |
| | | | | protein phosphatase) |
| 102a | CG59662-01 | 289 | 290 | Cyclophilin |
| 103a | CG59773-01 | 291 | 292 | Myomegalin |
| 103b | CG59773-02 | 293 | 294 | Myomegalin |
| 103c | CG59773-03 | 295 | 296 | Myomegalin |
| 104a | CG57460-01 | 297 | 298 | PEPTIDYL-PROLYL |
| | | | | CIS-TRANS |
| | | | | ISOMERASE like |
| 105a | CG57464-01 | 299 | 300 | N- |
| | | | | ACETYLTRANSFER |
| | | | | ASE like |
| 106a | CG57466-01 | 301 | 302 | Acetylglucosaminyl- |
| | | | | transferase like |
| 107a | CG57468-01 | 303 | 304 | ABC transporter like |
| | | | | homo sapiens |
| 108a | CG59609-01 | 305 | 306 | PEPTIDYL-PROLYL |
| | | | | CIS-TRANS |
| | | | | ISOMERASE A like |
| 109a | CG59613-01 | 307 | 308 | Proliferating cell |
| | | | | nuclear antigen like |
| 110a | CG59619-01 | 309 | 310 | CYTOPLASMIC |
| | | | | ACTIN 2 like |
| 111a | CG59621-01 | 311 | 312 | SELENOPHOSPHATE |
| | | | | SYNTHETASE like |
| 112a | CG59625-01 | 313 | 314 | glucose transporter like |
| 113a | CG59887-0l | 315 | 316 | Amino Acid/Metabolite |
| | | | | Permease like |
| 113b | CG59887-02 | 317 | 318 | Amino Acid/Metabolite |
| | | | | Permease like |
| 114a | CG59861-01 | 319 | 320 | RIBULOSE-5- |
| | | | | PHOSPHATE |
| | | | | EPIMERASE like |
| 114b | CG59861-02 | 321 | 322 | RIBULOSE-5- |
| | | | | PHOSPHATE |
| | | | | EPIMERASE like |
| 115a | CG59857-01 | 323 | 324 | Rhotekin like homo |
| | | | | sapiens |
| 116a | CG59855-0l | 325 | 326 | ATP SYNTHASE |
| | | | | SUBUNIT C lik |
| 116b | CG59855-02 | 327 | 328 | ATP SYNTHASE |
| | | | | SUBUNIT C like |
| 117a | CG59807-01 | 329 | 330 | Zinc finger like |
| 118a | CG59805-01 | 331 | 332 | Zinc finger like |
| 119a | CG59928-01 | 333 | 334 | Universal Stress (USP) |
| | | | | Domain Containing |
| | | | | Protein like |
| 120a | CG59947-01 | 335 | 336 | VOLTAGE-GATED |
| | | | | POTASSIUM |
| | | | | CHANNEL PROTEIN |
| | | | | KV3.3 (KSHIIID) like |
| 121a | CG59938-0l | 337 | 338 | arylsulfatase like homo |
| | | | | sapiens |
| 122a | CG59746-01 | 339 | 340 | ubiguitin-specific |
| | | | | processing protease |
| | | | | like homo sapiens |
| 123a | CG88613-01 | 341 | 342 | INOSITOL 1,4,5- |
| | | | | TRISPHOSPHATE 3- |
| | | | | KINASE |
| | | | | ISOENZYME like |
| 124a | CG59993-01 | 343 | 344 | synaptotagmin II like |
| 124b | CG59993-02 | 345 | 346 | synaptotagmin 11 like |
| 125a | CG59991-01 | 347 | 348 | ooplasm specific |
| | | | | protein like |
| 126a | CG59987-01 | 349 | 350 | GTP-RHO binding |
| | | | | protein 1 (rhophilin) |
| | | | | like |
| 126b | CG59987-02 | 351 | 352 | GTP-RHO binding |
| | | | | protein 1 (rhophilin) |
| | | | | like |
| 127a | CG59971-01 | 353 | 354 | Leucine rich repeat |
| | | | | (LRR) like |
| 127b | CG59971-02 | 355 | 356 | Leucine rich repeat |
| | | | | (LRR) like |

Table 1 indicates homology of NOVX nucleic acids to known protein families. Thus, the nucleic acids and polypeptides, antibodies and related compounds according to the invention corresponding to a NOVX as identified in column 1 of Table 1 will be useful in therapeutic and diagnostic applications implicated in, for example, pathologies and disorders associated with the known protein families identified in column 5 of Table 1.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table 1, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table 1.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. a variety of cancers.

Additional utilities for NOVX nucleic acids and polypeptides according to the invention are disclosed herein.

The present invention is based on the identification of biological macromolecules differentially modulated in a pathologic state, disease, or an abnormal condition or state. Among the pathologies or diseases of present interest include metabolic diseases including those related to endocrinologic disorders, cancers, various tumors and neoplasias, inflammatory disorders, central nervous system disorders, and similar abnormal conditions or states. In very significant embodiments of the present invention, the biological macromolecules implicated in the pathologies and conditions are proteins and polypeptides, and in such cases the present invention is related as well to the nucleic acids that encode them. Methods that may be employed to identify relevant biological macromolecules include any procedures that detect differential expression of nucleic acids encoding proteins and polypeptides associated with the disorder, as well as procedures that detect the respective proteins and polypeptides themselves. Significant methods that have been employed by the present inventors, include GeneCalling® technology and SeqCalling TM technology, disclosed respectively, in U.S. Pat. No. 5,871,697, and in U. S. Ser. No. 09/417,386, filed Oct. 13, 1999, each of which is incorporated herein by reference in its entirety. GeneCalling® is also described in Shimkets, et al., “Gene expression analysis by transcript profiling coupled to a gene database query” Nature Biotechnology 17:198-803 (1999).

The invention provides polypeptides and nucleotides encoded thereby that have been identified as having novel associations with a disease or pathology, or an abnormal state or condition, in a mammal. The present invention further identifies a set of proteins and polypeptides, including naturally occurring polypeptides, precursor forms or proproteins, or mature forms of the polypeptides or proteins, which are implicated as targets for therapeutic agents in the treatment of various diseases, pathologies, abnormal states and conditions. A target may be employed in any of a variety of screening methodologies in order to identify candidate therapeutic agents which interact with the target and in so doing exert a desired or favorable effect. The candidate therapeutic agent is identified by screening a large collection of substances or compounds in an important embodiment of the invention. Such a collection may comprise a combinatorial library of substances or compounds in which, in at least one subset of substances or compounds, the individual members are related to each other by simple structural variations based on a particular canonical or basic chemical structure. The variations may include, by way of nonlimiting example, changes in length or identity of a basic framework of bonded atoms; changes in number, composition and disposition of ringed structures, bridge structures, alicyclic rings, and aromatic rings; and changes in pendent or substituents atoms or groups that are bonded at particular positions to the basic framework of bonded atoms or to the ringed structures, the bridge structures, the alicyclic structures, or the aromatic structures.

A polypeptide or protein described herein, and that serves as a target in the screening procedure, includes the product of a naturally occurring polypeptide or precursor form or proprotein. The naturally occurring polypeptide, precursor or proprotein includes, e.g., the full-length gene product, encoded by the corresponding gene. The naturally occurring polypeptide also includes the polypeptide, precursor or proprotein encoded by an open reading frame described herein. A “mature” form of a polypeptide or protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a polypeptide or protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them.

As used herein, “identical” residues correspond to those residues in a comparison between two sequences where the equivalent nucleotide base or amino acid residue in an alignment of two sequences is the same residue. Residues are alternatively described as “similar” or “positive” when the comparisons between two sequences in an alignment show that residues in an equivalent position in a comparison are either the same amino acid or a conserved amino acid as defined below.

As used herein, a “chemical composition” relates to a composition including at least one compound that is either synthesized or extracted from a natural source. A chemical compound may be the product of a defined synthetic procedure. Such a synthesized compound is understood herein to have defined properties in terms of molecular formula, molecular structure relating the association of bonded atoms to each other, physical properties such as chromatographic or spectroscopic characterizations, and the like. A compound extracted from a natural source is advantageously analyzed by chemical and physical methods in order to provide a representation of its defined properties, including its molecular formula, molecular structure relating the association of bonded atoms to each other, physical properties such as chromatographic or spectroscopic characterizations, and the like.

As used herein, a “candidate therapeutic agent” is a chemical compound that includes at least one substance shown to bind to a target biopolymer. In important embodiments of the invention, the target biopolymer is a protein or polypeptide, a nucleic acid, a polysaccharide or proteoglycan, or a lipid such as a complex lipid. The method of identifying compounds that bind to the target effectively eliminates compounds with little or no binding affinity, thereby increasing the potential that the identified chemical compound may have beneficial therapeutic applications. In cases where the “candidate therapeutic agent” is a mixture of more than one chemical compound, subsequent screening procedures may be carried out to identify the particular substance in the mixture that is the binding compound, and that is to be identified as a candidate therapeutic agent.

As used herein, a “pharmaceutical agent” is provided by screening a candidate therapeutic agent using models for a disease state or pathology in order to identify a candidate exerting a desired or beneficial therapeutic effect with relation to the disease or pathology. Such a candidate that successfully provides such an effect is termed a pharmaceutical agent herein. Nonlimiting examples of model systems that may be used in such screens include particular cell lines, cultured cells, tissue preparations, whole tissues, organ preparations, intact organs, and nonhuman mammals. Screens employing at least one system, and preferably more than one system, may be employed in order to identify a pharmaceutical agent. Any pharmaceutical agent so identified may be pursued in further investigation using human subjects.

NOVX Nucleic Acids and Polypeptides

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 178; (b) a variant of a mature form of the amino acid sequence selected from the group consisting of SEQ ID NO: 2n, wherein n is an integer between 1 and 178, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 178; (d) a variant of the amino acid sequence selected from the group consisting of SEQ ID NO:2n, wherein n is an integer between 1 and 178 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 178; (b) a variant of a mature form of the amino acid sequence selected from the group consisting of SEQ ID NO: 2n, wherein n is an integer between 1 and 178 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 178; (d) a variant of the amino acid sequence selected from the group consisting of SEQ ID NO: 2n, wherein n is an integer between 1 and 178, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 178 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 178; (b) a nucleotide sequence wherein one or more nucleotides in the nucleotide sequence selected from the group consisting of SEQ ID NO: 2n−1, wherein n is an integer between 1 and 178 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 178; and (d) a nucleic acid fragment wherein one or more nucleotides in the nucleotide sequence selected from the group consisting of SEQ ID NO: 2n−1, wherein n is an integer between 1 and 178 is changed from that selected from the group consisting of the chosen sequence to a different nucleotide provided that no more than 15% of the nucleotides are so changed.

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NO: 2n−1, wherein n is an integer between 1 and 178, or a complement of this aforementioned nucleotide sequence, can be isolated using standard molecular biology techniques and the sequence information provided herein. Using all or a portion of the nucleic acid sequence of SEQ ID NO: 2n−1, wherein n is an integer between 1 and 178 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, erg., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NO: 2n−1, wherein n is an integer between 1 and 178, or a complement thereof. Oligonucleotides may be chemically synthesized and may also be used as probes.

In another embodiment, an isolated nucleic acid molecule of the invention comprises a nucleic acid molecule that is a complement of the nucleotide from the group consisting of SEQ ID NO: 2n−1, wherein n is an integer between 1 and 178, or a portion of this nucleotide sequence (e.g., a fragment that can be used as a probe or primer or a fragment encoding a biologically-active portion of an NOVX polypeptide). A nucleic acid molecule that is complementary to the nucleotide sequence from the group consisting of SEQ ID NO: 2n−1, wherein n is an integer between 1 and 178 is one that is sufficiently complementary to the nucleotide sequence from the group consisting of SEQ ID NO: 2n−1, wherein n is an integer between 1 and 178 that it can hydrogen bond with little or no mismatches to the nucleotide sequence from the group consisting of SEQ ID NO: 2n−1, wherein n is an integer between 1 and 178, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO: 2n−1, wherein n is an integer between 1 and 178, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NO: 2n−1, wherein n is an integer between 1 and 178; or an anti-sense strand nucleotide sequence of SEQ ID NO: 2n−1, wherein n is an integer between 1 and 178.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NO: 2n−1, wherein n is an integer between 1 and 178, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NO: 2n−1, wherein n is an integer between 1 and 178 due to degeneracy of the genetic code and thus encode the same NOVX proteins as that encoded by the nucleotide sequences shown in SEQ ID NO: 2n−1, wherein n is an integer between 1 and 178. In another embodiment, an isolated nucleic acid molecule of the invention has a nucleotide sequence encoding a protein having an amino acid sequence shown in SEQ ID NO: 2n, wherein n is an integer between 1 and 178.

In addition to the human NOVX nucleotide sequences shown in SEQ ID NO: 2n−1, wherein n is an integer between 1 and 178, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NO: 2n−1, wherein n is an integer between 1 and 178 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n−1, wherein n is an integer between 1 and 178. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at

pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NO: 2n−1, wherein n is an integer between 1 and 178, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n−1, wherein n is an integer between 1 and 178, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NO: 2n−1, wherein n is an integer between 1 and 178, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NO: 2n−1, wherein n is an integer between 1 and 178, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NO: 2n, wherein n is an integer between 1 and 178. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO: 2n−1, wherein n is an integer between 1 and 178 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NO: 2n, wherein n is an integer between 1 and 178. Preferably, the protein encoded by the nucleic acid molecule is at least about 60% homologous to SEQ ID NO: 2n, wherein n is an integer between 1 and 178; more preferably at least about 70% homologous SEQ ID NO: 2n, wherein n is an integer between 1 and 178; still more preferably at least about 80% homologous to SEQ ID NO: 2n, wherein n is an integer between 1 and 178; even more preferably at least about 90% homologous to SEQ ID NO: 2n, wherein n is an integer between 1 and 178; and most preferably at least about 95% homologous to SEQ ID NO: 2n, wherein n is an integer between 1 and 178.

An isolated nucleic acid molecule encoding an NOVX protein homologous to the protein of SEQ ID NO: 2n, wherein n is an integer between 1 and 178 can be created by introducing one or more nucleotide substitutions, additions or deletions into the nucleotide sequence of SEQ ID NO: 2n−1, wherein n is an integer between 1 and 178, such that one or more amino acid substitutions, additions or deletions are introduced into the encoded protein.

Mutations can be introduced into SEQ ID NO: 2n−1, wherein n is an integer between 1 and 178 standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NO: 2n−1, wherein n is an integer between 1 and 178, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1 and 178, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NO: 2n, wherein n is an integer between 1 and 178, or antisense nucleic acids complementary to an NOVX nucleic acid sequence of SEQ ID NO: 2n−1, wherein n is an integer between 1 and 178, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NO: 2n−1, wherein n is an integer between 1 and 178). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N. Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93:14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NO: 2n, wherein n is an integer between 1 and 178. The invention also includes a mutant or variant protein any of whose residues may be changed from the corresponding residues shown in SEQ ID NO: 2n, wherein n is an integer between 1 and 178 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NO: 2n, wherein n is an integer between 1 and 178) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NO: 2n, wherein n is an integer between 1 and 178. In other embodiments, the NOVX protein is substantially homologous to SEQ ID NO: 2n, wherein n is an integer between 1 and 178, and retains the functional activity of the protein of SEQ ID NO: 2n, wherein n is an integer between 1 and 178, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NO: 2n, wherein n is an integer between 1 and 178, and retains the functional activity of the NOVX proteins of SEQ ID NO: 2n, wherein n is an integer between 1 and 178.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA from the group consisting of SEQ ID NO: 2n−1, wherein n is an integer between 1 and 178.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an A NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NO: 2n, wherein n is an integer between 1 and 178, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

NOVX Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 2n, wherein n is an integer between 1 and 178, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. Nature 368 856-859 (1994)); Morrison (Nature 368, 812-13 (1994)); Fishwild et al, (Nature Biotechnology 1, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, 131i, ¹³¹In, ⁹⁰Y, and ¹⁸Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

Antibodies directed against a protein of the invention may be used in methods known within the art relating to the localization and/or quantitation of the protein (e.g., for use in measuring levels of the protein within appropriate physiological samples, for use in diagnostic methods, for use in imaging the protein, and the like). In a given embodiment, antibodies against the proteins, or derivatives, fragments, analogs or homologs thereof, that contain the antigen binding domain, are utilized as pharmacologically-active compounds (see below).

An antibody specific for a protein of the invention can be used to isolate the protein by standard techniques, such as immunoaffinity chromatography or immunoprecipitation. Such an antibody can facilitate the purification of the natural protein antigen from cells and of recombinantly produced antigen expressed in host cells. Moreover, such an antibody can be used to detect the antigenic protein (e.g., in a cellular lysate or cell supernatant) in order to evaluate the abundance and pattern of expression of the antigenic protein. Antibodies directed against the protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSecI (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banedji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NO: 2n−1, wherein n is an integer between 1 and 178 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NO: 2n−1, wherein n is an integer between 1 and 178), but more preferably, is a non-human homologue of a human NOVX gene. For example, a mouse homologue of human NOVX gene of SEQ ID NO: 2n−1, wherein n is an integer between 1 and 178 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TETRATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, orcan comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (ie., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GALA). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NO: 2n−1, wherein n is an integer between 1 and 178, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal 0.4 z spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO: 2n−1, wherein n is an integer between 1 and 178 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO: 2n−1, wherein n is an integer between 1 and 178, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (ie., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g. a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome PREGNANCY ZONE PROTEIN PRECURSOR enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C 19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification. Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials.

For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (ie., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

Sequence Analyses

The sequence of NOVX was derived by laboratory cloning of cDNA fragments, by in silico prediction of the sequence. cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The laboratory cloning was performed using one or more of the methods summarized below:

SeqCalling™Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen Corporation's SeqCalling technology which is disclosed in full in U.S. Ser. No. 09/417,386 filed Oct. 13, 1999, and Ser. No. 09/614,505 filed Jul. 11, 2000. Sequence traces were evaluated manually and edited for corrections if appropriate. cDNA sequences from all samples were assembled together, sometimes including public human sequences, using bioinformatics programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

Presented information includes that associated with genomic clones, public genes and ESTs sharing sequence identity with the disclosed sequence and CuraGen Corporation's Electronic Northern bioinformatic tool.

### EXAMPLES

### Example A

### Sequence related information

The NOV1 clone was analyzed, and the nucleotide and predicted polypeptide sequences are shown in Table 1A.

TABLE 1A
NOV1 Sequence Analysis

| | SEQ ID NO:1 | 711 bp | |
|---|---|---|---|
| NOV1a, | TGCAGAATGAACCAAGGAGACTCAAACCCAGCAGCTACTCCGCATGCGGCAGAAGACA | | |
| CG582522-01 DNA Sequence | | | |
| | TTCAAGGAGATGACAGATGGATGTGTCAGCACAACAGATTTGTTTTGGACTGTAAAGA | | |
| | CAAACAGCCTGATGTACCATTTGCGGGAGGCTCCGTGGTGCAGTTACTGCAGCCATAT | | |
| | GAGATATGGCGAGAGCTTTTTTCCCCACTTCATGCACTGAATTTTGGAACTGGGGGAG | | |
| | ATACAACAAGACATGTTTTGTGGAGACTAAAGAGCGGAGAACTGGGGAATACTAAGCC | | |
| | TAAGGTCATTGTTTTCTGGCTAGGAAGAAACAACCATGAAAATATGGCAGAAGAGGTA | | |
| | GCAGGTGGTATGGCGGCCATCGTACAACTTATCAACACAAGGCAGCCACAGGCCAAAA | | |
| | TCATTGTATTTGATCTGTTACCTCAAGGTGAGAAACCCAACCCTTTGAGGCAAAAGAA | | |
| | CGCCAAGGTGAACCCACTCGTCAAGATTTCGCTGCTGAAACTTACCAACGTGCAGCTC | | |
| | CTGGATACTGACAGGGGTTTCGTGCACTCCGACCGTGCCATCTCCTGCCACGACATGT | | |
| | TTGATTTTCTGCATTTGACAGGAGGTGGCTACTCAAAGGTCTGCAAACCCTTGAATGA | | |
| | ACTGATCATGCAGTTGTTGGAGGAAACACCTGAGGAGAAACAAACCACCATTGCCTGA | | |
| | CTGGCTCCCATGAGT | | |
| | ORF STart: ATG at 7 | | ORF Stop: TGA at 694 |
| | SEQ ID NO:2 | 229 aa | MW at 25656.2 kD |

| | |
|---|---|
| NOV1a, | MNQGDSNPAATPHAAEDIQGDDRWMCQHNRFVLDCKDKQPDVPFAGGSVVQLLQPYEI |
| CG58522-01 Protein Sequence | |
| | WRELFSPLHALNFGTGGDTTRHVLWRLKSGELGNTKPKVIVFWLGRNMAEEVAG |
| | GMAAIVQLINTRQPQAKIIVFDLLPQGEKPNPLRQKNAKVNPLVKISLLKLTNVQLLD |
| | TDRGFVHSDRAISCHDMFDELHLTGGGYSKVCKPLNELIMQLLEETPEEEQTTIA |

Further analysis of the NOV1a protein yielded the following properties shown in Table 1B.

TABLE 1B
Protein Sequence Properties NOV1a

| | |
|---|---|
| Psort | 0.6500 probability located in cytoplasm; 0.2340 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV1a protein against the Geneseq database, a proprietary database that contains sequences published in patents and patent publication, yielded several homologous proteins shown in Table 1C.

TABLE 1C
Geneseq Results for NOV1a

| | | | Identities/ | |
|---|---|---|---|---|
| | | NOV1a | Similarites | |
| | | Residues/ | for the | |
| Geneseq | Protein/Organism/Length | Match | Matched | Expect |
| Identifier | [Patent #, Date] | Residues | Region | Value |
| AAB49433 | Human beta platelet | 1 . . . 229 | 196/229 | e−114 |
| | activating factor | 1 . . . 229 | (85%) | |
| | acetylhydrolase - Homo | | 209/229 | |
| | sapiens, 229 aa. [U.S. | | (90%) | |
| | Pat. No. 6146868-A, | | | |
| | 14 NOV. 2000] | | | |
| AAB49432 | Rat beta platelet | 1 . . . 229 | 195/229 | e−114 |
| | activating factor | 1 . . . 229 | (85%) | |
| | acetylhydrolase - Rattus | | 208/229 | |
| | norvegicus, 229 aa. [U.S. | | (90%) | |
| | Pat. No. 6146868-A, | | | |
| | 14 NOV. 2000] | | | |
| AAB49434 | Murine beta platelet | 1 . . . 229 | 192/229 | e−111 |
| | activating factor | 1 . . . 229 | (83%) | |
| | acetylhydrolase - Mus | | 205/229 | |
| | musculus, 229 aa. [U.S. | | (88%) | |
| | Pat. No. 6146868-A, | | | |
| | 14 NOV. 2000] | | | |
| AAB49436 | Bovine gamma platelet | 4 . . . 219 | 124/216 | 5e−74 |
| | activating factor | 3 . . . 218 | (57%) | |
| | acetylhydrolase - Bos | | 165/216 | |
| | taurus, 1232 aa. [U.S. | | (75%) | |
| | Pat. No. 6146868-A, | | | |
| | 14 NOV. 2000] | | | |
| AAB49435 | Human gamma platelet | 4 . . . 219 | 124/216 | 2e−73 |
| | activating factor | 3 . . . 218 | (57%) | |
| | acetylhydrolase - Homo | | 164/216 | |
| | sapiens, 231 aa. [U.S. | | (75%) | |
| | Pat. No. 6146868-A, | | | |
| | 14 NOV. 2000] | | | |

In a BLAST search of public sequence databases, the NOV1a protein was found to have homology to the proteins shown in the BLASTP data in Table 1D.

TABLE 1D
Public BLASTP Results for NOV1a

| | | NOV1a | Identities/ | |
|---|---|---|---|---|
| Protein | | Residues/ | Similarities for | |
| Accession | Protein/Organism/ | Match | the Matched | Expect |
| Number | Length | Residues | Portion | Value |
| Q29459 | Platelet-activating | 1 . . . 229 | 196/229 (85%) | e−114 |
| | factor acetylhydrolase | 1 . . . 229 | 209/229 (90%) | |
| | IB beta subunit | | | |
| | (EC 3.1.1.47) (PAF | | | |
| | acetylhydrolase 30 | | | |
| | kDa subunit) | | | |
| | (PAF-AH 30 kDa | | | |
| | subunit) (PAF-AH | | | |
| | beta subunit) | | | |
| | (PAFAH beta | | | |
| | subunit) - Homo | | | |
| | sapiens (Human), | | | |
| | and, 229 aa. | | | |
| O35264 | Platelet-activating | 1 . . . 229 | 195/229 (85%) | e−113 |
| | factor acetylhydrolase | 1 . . . 229 | 208/229 (90%) | |
| | IB beta subunit (EC | | | |
| | 3.1.1.47) (PAF | | | |
| | acetylhydrolase 30 | | | |
| | kDa subunit) | | | |
| | (PAF-AH beta | | | |
| | subunit) (PAFAH | | | |
| | beta subunit) | | | |
| | (Platelet- | | | |
| | activating factor | | | |
| | acetylhydrolase alpha, | | | |
| | 2 subunit) (PAF-AH | | | |
| | alpha 2) - Rattus | | | |
| | norvegicus (Rat), | | | |
| | 229 aa. | | | |
| Q61206 | Platelet-activating | 1 . . . 229 | 192/229 (83%) | e−111 |
| | factor acetylhydrolase | 1 . . . 229 | 205/229 (88%) | |
| | IB beta subunit (EC | | | |
| | 3.1.1.47) (PAF | | | |
| | acetylhydrolase 30 | | | |
| | kDa subunit) | | | |
| | (PAF-AH 30 kDa | | | |
| | subunit) (PAF-AH | | | |
| | beta subunit) | | | |
| | (PAFAH beta | | | |
| | subunit) - Mus | | | |
| | musculus (Mouse), | | | |
| | 229 aa. | | | |
| Q29460 | Platelet-activating | 4 . . . 219 | 125/216 (57%) | 8e−74 |
| | factor acetylhydrolase | 3 . . . 218 | 165/216 (75%) | |
| | IB gamma subunit | | | |
| | (EC 3.1.1.47) | | | |
| | (PAF acetylhydrolase | | | |
| | 29 kDa subunit) | | | |
| | (PAF-AH 29 kDa | | | |
| | subunit) (PAF-AH | | | |
| | gamma subunit) | | | |
| | (PAFAH gamma | | | |
| | subunit) - Bos taurus | | | |
| | (Bovine), 232 aa. | | | |
| Q15102 | Platelet-activating | 4 . . . 219 | 124/216 (57%) | 7e−73 |
| | factor acetyl- | 3 . . . 218 | 164/216 (75%) | |
| | hydrolase IB | | | |
| | gamma subunit | | | |
| | (EC 3.1.1.47) | | | |
| | (PAF acetylhydrolase | | | |
| | 29 kDa subunit) | | | |
| | (PAF-AH 29 kDa | | | |
| | subunit) (PAF-AH | | | |
| | gamma subunit) | | | |
| | (PAFAH gamma | | | |
| | subunit) - Homo | | | |
| | sapiens (Human), | | | |
| | 231 aa. | | | |

PFam analysis predicts that the NOV1a protein contains the domains shown in the Table 1E.

TABLE 1E
Domain Analysis of NOV1a

| | NOV1a | Identities/Similarities | Expect |
|---|---|---|---|
| Pfam Domain | Match Region | for the Matched Region | Value |
| PAF-AH: | 7 . . . 221 | 150/215 (70%) | 6e−147 |
| domain 1 of 1 | | 186/215 (87%) | |

### Example 2

The NOV2 clone was analyzed, and the nucleotide and predicted polypeptide sequences are shown in Table 2A.

TABLE 2A
NOV2 Sequence Analysis

| | SEQ ID NO:3 | 1457 bp | |
|---|---|---|---|
| NOV2a, | CGATTCCGATGGGTCCTTTGAAAGCTTTTCTCTTCTCCCCTTTTCTTCTGCGGAGTCA | | |
| CG58520-01 DNA Sequence | | | |
| | AAGTAGAGGGGTGAGGTTGGTCTTCTTGTTACTGACCCTGCATTTGGGAAACGTTGAT | | |
| | AAGGCAGATGATGAAGATGATGAGGATTTAACGGTGAACAAAACCTGGGTCTTGGCCC | | |
| | CAAAAATTCATGAAGGAGATATCACACAAATTCTGAATTCATTGCTTCAAGGCTATGA | | |
| | CAATAAACTTCGTCCAGATATAGGAGTGAGGCCCACAGTAATTGAAACTGATGTTTAT | | |
| | GTAAACAGCATTGGACCAGTTGATCCAATTAATATGGAATATACAATAGATATAATTT | | |
| | TTGCCCAAACCTGGTTTGACAGTCGTTTAAAATTCAATAGTACCATGAAAGTGCTTAT | | |
| | GCTTAACAGTAATATGGTTGGAAAAATTTGGATTCCTGACACTTTCTTCAGAAACTCA | | |
| | AGAAAATCTGATGCTCACTGGATAACAACTCCTAATCGTCTGCTTCGAATTTGGAATG | | |
| | ATAGATGGATACCCTAAAAATGAAATTGAGTTATCAATGGAAGCGAAGTTCTGTGGAA | | |
| | GTGGGCGACACAAGATCCGGAGATTATATCAGTTTGCATTTGTAGGGTTACGGAACTC | | |
| | AACTGAAATCACTCACACGATCTCTGGGGATTATGTTATCATGACAATTTTTTTTGAC | | |
| | CTGAGCAGAAGAATGGGATATTTCACTATTCAGACCTACATTCCATGCATTCTGACAG | | |
| | TTGTTCTTTCTTGGGTGTCTTTTTGGATCAATAAAGATGCAGTGCCTGCAAGAACATC | | |
| | GTTGGGTATGACATCTATAGGTATCACTACAGTTCTGACTATGACAACCCTGAGTACA | | |
| | ATTGCCAGGAAGTCTTTACCTAAGGTTTCTTATGTGACTGCGATGGATCTCTTTGTTT | | |
| | CTGTTTGTTTCATTTTTGTTTTTGCAGCCTTGATGGAATATGGAACCTTGCATTATTT | | |
| | TACCAGCAACCAAAAAGGAAAGACTGCTACTAAAGACAGAAAGCTAAAAAATAAAGCC | | |
| | TCGACTCCTGGTCTCCATCCTGGATCCACTCTGATTCCAATGAATAATATTTCTGTGC | | |
| | CGCAAGAAGATGATTATGGGTATCAGTGTTTGGAGGGCAAAGATTGTGCCAGCTTCTT | | |
| | CTGTTGCTTTGAAGACTGCAGAACAGGATCTTGGAGGGAAGGAAGGATACACATACGC | | |
| | ATTGCCAAAATTGACTCTTATTCTAGAATATTTTTCCCAACCGCTTTTGCCCTGTTCA | | |
| | ACTTGGTTTATTGGGTTGGCTATCTTTACTTATAAAATCTACTTCATAAGCAAAAATC | | |
| | AAAAGAA | | |
| | ORF Start: ATG at 9 | | ORF Stop: TAA at 1425 |
| | SEQ ID NO:4 | 472 aa | MW at 54100.9 kD |
| NOV2a, | MGPLKAFLFSPELLRSQSRGVRLVFLLLTLHLGNVDKADDEDDEDLTVNKTWVLAPKI | | |
| CG58520-01 Protein Sequence | | | |
| | HEGDITQILNSLLQGYDNKLRPDIGVRPTVIETDVYVNSIGPVDPINMEYTIDIIFAQ | | |
| | TWFDSRLKFNSTMKVLMLNSNMVGKIWIPDTFFRNSRKSDAHWITTPNRLLRIWNDGR | | |
| | VLYTLRRLTINAECYLQLHNFPMDEHSCPLEFSSFSIDGYPKNEIELSMEAKFCGSGR | | |
| | HKIRRLYQFAFVGLRNSTEITHTISGDYVIMTIFFDLSRRMGYFTIQTYIPCILTVVL | | |
| | SWVSFWINKDAVPARTSLGMTSIGITTVLTMTTLSTIARKSLPKVSYVTAMDLFVSVC | | |
| | FIFVFAALMEYGTLHYFTSNQKGKTATKDRKLKNKASTPGLHPGSTLIPMNNISVPQE | | |
| | DDYGYQCLEGKDCASFFCCFEDCRTGSWREGRIHIRIAKIDSYSRIFFPTAFALFNLV | | |
| | YWVGYLYL | | |
| | SEQ ID NO:5 | 1521 bp | |
| NOV2b, | CAACCAAGAGGCAAGAGGCGAGAGAAGGAAAAAAAAAAAAGCGATGAGTTCGCCAAAT | | |
| CG58520-02 DNA Sequence | | | |
| | ATATGGAGCACAGGAAGCTCAGTCTACTCGACTCCTGTATTTTCACAGAAAATGACGG | | |
| | TGTGGATTCTGCTCCTGCTGTCGCTCTACCCTGGCTTCACTAGCCAGAAATCTGATGA | | |
| | TGACTATGAAGATTATGCTTCTAACAAAACATGGGTCTTGACTCCAAAAGTTCCTGAG | | |
| | GGTGATGTCACTGTCATCTTAAACAACCTGCTGGAAGGATATGACAATAAACTTCGGC | | |
| | CTGATATAGGAGTGAAGCCAACGTTAATTCACACAGACATGTATGTGAATAGCATTGG | | |
| | TCCAGTGAACGCTATCAATATGGAATACACTATTGATATATTTTTTGCGCAAACGTGG | | |
| | TATGACAGACGTTTGAAATTTAACAGCACCATTAAAGTCCTCCGATTGAACAGCAACA | | |
| | TGGTGGGGAAAATCTGGATTCCAGACACTTTCTTCAGAAATTCCAAAAAAGCTGATGC | | |
| | ACACTGGATCACCACCCCCAACAGGATGCTGAGAATTTGGAATGATGGTCGAGTGCTC | | |
| | TACACCCTAAGGTTGACAATTGATGCTGAGTGCCAATTACAATTGCACAACTTTCCAA | | |
| | TGGATGAACACTCCTGCCCCTTGGAGTTCTCCAGTTATGGCTATCCACGTGAAGAAAT | | |
| | TGTTTATCAATGGAAGCGAAGTTCTGTTGAAGTGGGCGACACAAGATCCTGGAGGCTT | | |
| | TATCAATTCTCATTTGTTGGTCTAAGAAATACCACCGAAGTAGTGAAGACAACTTCCG | | |
| | GAGATTATGTGGTCATGTCTGTCTACTTTGATCTGAGCAGAAGAATGGGATACTTTAC | | |
| | CATCCAGACCTATATCCCCTGCACACTCATTGTCGTCCTATCCTGGGTGTCTTTCTGG | | |
| | ATCAATAAGGATGCTGTTCCAGCCAGAACATCTTTAGGTATCACCACTGTCCTGACAA | | |
| | TGACCACCCTCAGCACCATTGCCCGGAAATCGCTCCCCAAGGTCTCCTATGTCACAGC | | |
| | GATGGATCTCTTTGTATCTGTTTGTTTCATCTTTGTCTTCTCTGCTCTGGTGGAGTAT | | |
| | GGCACCTTGCATTATTTTGTCAGCAACCGGAAACCAAGCAAGGACAAAGATAAAAAGA | | |
| | AGAAAAACCCTCTTCTTCGGATGTTTTCCTTCAAGGCCCCTACCATTGATATCCGCCC | | |
| | AAGATCAGCAACCATTCAAATGAATAATGCTACACACCTTCAAGAGAGAGATGAAGAG | | |
| | TACGGCTATGAGTGTCTGGACGGCAAGGACTGTGCCAGTTTTTTCTGCTGTTTTGAAG | | |
| | ATTGTCGAACAGGAGCTTGGAGACATGGGAGGATACATATCCGCATTGCCAAAATGGA | | |
| | CTCCTATGCTCGGATCTTCTTCCCCACTGCCTTCTGCCTGTTTAATCTGGTCTATTGG | | |
| | GTCTCCTACCTCTACCTGTGAGGAGGTATGGGTTTTACTGATATGGTTCTTATTCACT | | |
| | GAGTCTCATGGAG | | |
| | ORF Start ATG at 44 | | ORF Stop: TGA at 1469 |
| | SEQ ID NO:6 | 475 aa | MW at 55184.9 kD |
| NOV2b, | MSSPNIWSTGSSVYSTPVFSQKMTVWILLLLSLYPGFTSQKSDDDYEDYASNKTWVLT | | |
| CG58520-02 Protein Sequence | | | |
| | PKVPEGDVTVILNNLLEGYDNKLRPDIGVKPTLIHTDMYVNSIGPVNAINMEYTIDIF | | |
| | FAQTWYDRRLKFNSTIKVLRLNSNMVGKIWIPDTFFRNSKKADAHWITTPNRMLRIWN | | |
| | DGRVLYTLRLTIDAECQLQLHNFPMDEHSCPLEFSSYGYPREEIVYQWKRSSVEVGDT | | |
| | RSWRLYQFSFVGLRNTTEVVKTTSGDYVVMSVYFDLSRRMGYFTIQTYIPCTLIVVLS | | |
| | WVSFWINKDAVPARTSLGITTVLTMTTLSTIARKSLPKVSYVTAMDLFVSVCFIFVFS | | |
| | ALVEYGTLHYFVSNRKPSKDKDKKKKNPLLRMFSFKAPTIDIRPRSATIQMNNATHLQ | | |
| | ERDEEYGYECLDGKDCASFFCCFEDCRTGAWRHGRIHIRIAKMDSYARIFFPTAFCLF | | |
| | NLVYWVSYLYL | | |
| | SEQ ID NO:7 | 1455 bp | |
| NOV2c, | TAGTGCAGCACACGTAAAAAAGCGATTCCGATGGGTCCTTTGAAAGCTTTTCTCTTCT | | |
| CG58520-03 DNA Sequence | | | |
| | CCCCTTTTCTTCTGCGGAGTCAAAGTAGAGGGGTGAGGTTGGTCTTCTTGTTACTGAC | | |
| | CCTGCATTTGGGAAACTGGGTTGATAAGGCAGATGATGAAGATGATGAGGATTTAACG | | |
| | GTGAACAAAACCTGGGTCTTGGCCCCAAAAATTCATGAAGGAGATATCACACAAATTC | | |
| | TGAATTCATTGCTTCAAGGCTATGACAATAAACTTCGTCCAGATATAGGAGTGAGGCC | | |
| | CACAGTAATTGAAACTGATGTTTATGTAAACAGCATTGGACCAGTTGATCCAATTAAT | | |
| | ATGGAATATACAATAGATATAATTTTTGCCCAAACCTGGTTTGACAGTCGTTTAAAAT | | |
| | TCAATAGTACCATGAAAGTGCTTATGCTTAACAGTAATATGGTTGGAAAAATTTGGAT | | |
| | TCCTGACACTTTCTTCAGAAACTCAAGAAAATCTGATGCTCACTGGATAACAACTCCT | | |
| | AATCGTCTGCTTCGAATTTGGAATGATGGACGAGTTCTGTATACTCTAAGGTTGACAA | | |
| | TTAATGCAGAATGTTATCTTCAGCTTCATAACTTTCCCATGGATGAACATTCCTGTCC | | |
| | ACTGGAATTTTCAAGCGATGGATACCCTAAAAATGAAATTGAGTATAAGTGGAAAAAG | | |
| | CCCTCCGTAGAAGTGGCTGATCCTAAATACTGGAGATTATATCAGTTTGCATTTGTAG | | |
| | GGTTACGGAACTCAACTGAAATCACTCACACGATCTCTGGTGATTATGTTATCATGAC | | |
| | AATTTTTTTTGACCTGAGCAGAAGAATGGGATATTTCACTATTCAGACCTACATTCCA | | |
| | TGCATTCTGACAGTTGTTCTTTCTTGGGTGTCTTTTTGGATCAATAAAGATGCAGTGC | | |
| | CTGCAAGAACATCGTTGGGTATCACTACAGTTCTGACTATGACAACCCTGAGTACAAT | | |
| | TGCCAGGAAGTCTTTACCTAAGGTTTCTTATGTGACTGCGATGGATCTCTTTGTTTCT | | |
| | GTTTGTTTCATTTTTGTTTTTGCAGCCTTGATGGAATATGGAACCTTGCATTATTTTA | | |
| | CCAGCAACCAAAAAGGAAAGACTGCTACTAAAGACAGAAAGCTAAAAAATAAAGCCTC | | |
| | GGTAACTCCTGGTCTCCATCCTGGATCCACTCTGATTCCAATGAATAATATTTCTGTG | | |
| | CCGCAAGAAGATGATTATGGGTATCAGTGTTTGGAGGGCAAAGATTGTGCCAGCTTCT | | |
| | TCTGTTGCTTTGAAGACTGCAGAACAGGATCTTGGAGGGAAGGAAGGATACACATACG | | |
| | CATTGCCAAAATTGACTCTTATTCTAGAATATTTTTCCCAACCGCTTTTGCCCTGTTC | | |
| | AACTTGGTTTATTGGGTTGGCTATCTTTTACTTATAAAATCTACTTCATAAGCAAAAAT | | |
| | CAAAA | | |
| | ORF Start: ATG at 31 | | ORF Stop: TAA at 1426 |
| | SEQ ID NO:8 | 465 aa | MW at 53597.3 kD |

| | |
|---|---|
| NOV2c, | MGPLKAFLFSPFLLRSQSRGVRLVFLLLTLHLGNWVDKADDEDDEDLTVNKTWVLAPK |
| CG58520-03 Protein Sequence | |
| | IHEGDITQILNSLLQGYDNKLRPDIGVRPTVIETDVYVNSIGPVDPINMEYTIDIIFA |
| | QTWFDSRLKFNSTMKVLMLNSNMVGKIWIPDTFFRNSRKSDAHWITTPNRLLRIWNDG |
| | RVLYTLRLTINAECYLQLHNFPMDEHSCPLEFSSDGYPKNEIEYKWKKPSVEVADPKY |
| | WRLYQFAFVGLRNSTEITHTISGDYVIMTIFFDLSRRMGYFTIQTYIPCILTVVLSWV |
| | SFWINKDAVPARTSLGITTVLTMTTLSTIARKSLPKVSYVTAMDLFVSVCFIFVFAAL |
| | MEYGTLHYFTSNQKGKTATKDRKLKNKASVTPGLHPGSTLIPMNNISVPQEDDYGYQC |
| | LEGKDCASFFCCFEDCRTGSWREGRIHIRIAKIDSYSRIFFPTAFALFNLVYWVGYLYL |

Sequence comparison of the above protein sequences yields the following sequence relationships shown in Table 2B.

TABLE 2B
Comparison of NOV2a against NOV2b through NOV2c.

| Protein | NOV2a Residues/ | Identities/ |
|---|---|---|
| Sequence | Match Residues | Similarities for the Matched Region |
| NOV2b | 24 . . . 472 | 311/458 (67%) |
| | 27 . . . 475 | 352/458 (75%) |
| NOV2c | 1 . . . 472 | 414/474 (87%) |
| | 1 . . . 465 | 415/474 (87%) |

Further analysis of the NOV2a protein yielded the following properties shown in Table 2C.

TABLE 2C
Protein Sequence Properties NOV2a

| | |
|---|---|
| Psort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Likely cleavage site between residues 38 and 39 |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | | Identities/ | |
|---|---|---|---|---|
| | | NOV2a | Similarites | |
| | Protein/ | Residues/ | for the | |
| Geneseq | Organism/Length | Match | Matched | Expect |
| Identifier | [Patent #, Date] | Residues | Region | Value |
| AAM41007 | Human polypeptide | 24 . . . 472 | 334/451 | 0.0 |
| | SEQ ID NO 5938 - | 49 . . . 489 | (74%) | |
| | Homo sapiens, 489 aa. | | 379/451 | |
| | [WO200153312-A1, | | (83%) | |
| | 26 JUL. 2001] | | | |
| AAM39221 | Human polypeptide | 24 . . . 472 | 334/451 | 0.0 |
| | SEQ ID NO 2366 | | (74%) | |
| | Homo sapiens, 467 aa. | 27 . . . 467 | 379/451 | |
| | [WO200153312-A1, | | (83%) | |
| | 26 JUL. 2001] | | | |
| AAR83968 | GABA-A receptor | 24 . . . 472 | 300/472 | e−169 |
| | gamma-3 subunit - | 5 . . . 467 | (63%) | |
| | Homo sapiens, 467 aa. | | 356/472 | |
| | [WO9529234-A1, | | (74%) | |
| | 02 NOV. 1995] | | | |
| AAW59048 | GABA-A receptor | 62 . . . 472 | 193/448 | e−102 |
| | epsilon sub-unit | 70 . . . 506 | (43%) | |
| | related protein - | | 274/448 | |
| | Mammalia, 506 aa. | | (61%) | |
| | [DE19644501-A1, | | | |
| | 30 APR. 1998] | | | |
| AAW61045 | Human GABA | 62 . . . 472 | 193/448 | e−102 |
| | receptor epsilon | 70 . . . 506 | (43%) | |
| | subunit - Homo | | 274/448 | |
| | sapiens, 506 aa. | | (61%) | |
| | [WO9823742-A1, | | | |
| | 04 JUN. 1998] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | Protein/Organism/ | Match | the Matched | Expect |
| Number | Length | Residues | Portion | Value |
| P23574 | Gamma-amino- | 1 . . . 472 | 426/475 (89%) | 0.0 |
| | butyric-acid | 1 . . . 465 | 440/475 (91%) | |
| | receptor gamma-1 | | | |
| | subunit precursor | | | |
| | (GABA(A) | | | |
| | receptor) - | | | |
| | Rattus norvegicus | | | |
| | (Rat), 465 aa. | | | |
| Q9R0Y8 | Gamma-amino- | 1 . . . 472 | 420/477 (88%) | 0.0 |
| | butyric-acid | 1 . . . 465 | 434/477 (90%) | |
| | receptor gamma-1 | | | |
| | subunit precursor | | | |
| | (GABA(A) | | | |
| | receptor) - | | | |
| | Mus musculus | | | |
| | (Mouse), 465 aa. | | | |
| JH0824 | gamma-amino- | 16 . . . 472 | 390/463 (84%) | 0.0 |
| | butyric acid A | 12 . . . 464 | 416/463 (89%) | |
| | receptor gamma 1 | | | |
| | chain precursor - | | | |
| | chicken, 464 aa. | | | |
| JH0316 | gamma-amino- | 24 . . . 472 | 336/451 (74%) | 0.0 |
| | butyric acid A | 26 . . . 466 | 380/451 (83%) | |
| | receptor gamma 2 | | | |
| | chain alternatively | | | |
| | spliced precursor - | | | |
| | mouse, 466 aa. | | | |
| P18508 | Gamma-amino- | 24 . . . 472 | 335/451 (74%) | 0.0 |
| | butyric-acid receptor | 26 . . . 466 | 379/451 (83%) | |
| | gamma-2 subnuit | | | |
| | precursor | | | |
| | (GABA(A) | | | |
| | receptor) - | | | |
| | Rattus norvegicus | | | |
| | (Rat), 466 aa. | | | |

PFam analysis predicts that the NOV2a protein contains the domains shown in the Table 2F.

TABLE 2F
Domain Analysis of NOV2a

| | NOV2a | | |
|---|---|---|---|
| | Match | Identities/Similarities | Expect |
| Pfam Domain | Region | for the Matched Region | Value |
| Neur_chan_LBD: | 63 . . . 273 | 66/271 (24%) | 2.7e−56 |
| domain 1 of 1 | | 162/271 (60%) | |
| Cys-protease-3C: | 363 . . . 369 | 4/7 (57%) | 5.2 |
| domain 1 of 1 | | 6/7 (86%) | |
| Neur_chan_memb: | 280 . . . 466 | 44/297 (15%) | 1.2e−60 |
| domain 1 of 1 | | 164/297 (55%) | |

### Example 3

The NOV3 clone was analyzed, and the nucleotide and predicted polypeptide sequences are shown in Table 3A.

TABLE 3A
NOV3 Sequence Analysis

| | SEQ ID NO:9 | 1440 bp | |
|---|---|---|---|
| NOV3a, | GAAGAGATGGTCCTGGCTTTCCAGTTAGTCTCCTTCACCTACATCTGGATCATATTGA | | |
| CG58518-01 DNA Sequence | | | |
| | AACCAAATGTTTGTGCTGCTTCTAACATCAAGATGACACACCAGCGGTGCTCCTCTTC | | |
| | AATGAAACAAACCTGGAAACAAGAAACTAGAATGAAGAAAGATGACAGTACCAAAGCG | | |
| | CGGCCTCAGAAATATGAGCAACTTCTCCATATAGAGGACAACGATTTCGCAATGAGAC | | |
| | CTGGATTTGGAGGTGAGTATTATCCTCTCAAAATTGGGTCTCCAGTGCCAGTAGGTAT | | |
| | AGATGTCCATGTTGAAAGCATTGACAGCATTTCAGAGACTAACATGGTAAGTTTCTTC | | |
| | ATGGGATATGACTTTACAATGACTTTTTATCTCAGGCATTACTGGAAAGACGAGAGGC | | |
| | TCTCCTTTCCTAGCACAGCAAACAAAAGCATGACATTTGATCATAGATTGACCAGAAA | | |
| | GATCTGGGTGCCTGATATCTTTTTTGTCCACTCTAAAAGATCCTTCATCCATGATACA | | |
| | ACTATGGAGAATATCATGCTGCGCGTACACCCTGATGGAAACGTCCTCCTAAGTCTCA | | |
| | GGAGGATAACGGTTTCGGCCATGTGCTTTATGGATTTCAGCAGGTTTCCTCTTGACAC | | |
| | TCAAAATTGTTCTCTTGAACTGGAAAGCGCCTACAATGAGGATGACCTAATGCTATAC | | |
| | TGGAAACACGGAAACAAGTCCTTAAATACTGAAGAACATATGTCCCTTTCTCAGTTCT | | |
| | TCATTGAAGACTTCAGTGCATCTAGTGGATTAGCTTTCTATAGCAGCACAGGTTGGTA | | |
| | CAATAGGCTTTTCATCAACTTTGTGCTAAGGAGGCATGTTTTCTTCTTTGTGCTGCAA | | |
| | ACCTATTTCCCAGCCATATTGATGGTGATGCTTTCATGGGTTTCATTTTGGATTGACC | | |
| | GAAGAGCTGTTCCTGCAAGAGTTTCCCTGGGTGGAATCACCACAGTGCTGACCATGTC | | |
| | CACAATCATCACTGCTGTGAGCGCCTCCATGCCCCAGGTGTCCTACCTCAAGGCTGTG | | |
| | GATGTGTACCTGTGGGTCAGCTCCCTCTTTGTGTTCCTGTCAGTCATTGAGTATGCAG | | |
| | CTGTGAACTACCTCACCACAGTGGAAGAGCGGAAACAATTCAAGAAGACAGGAAAGGT | | |
| | ACAGATTTCTAGGATGTACAATATTGATGCAGTTCAAGCTATGGCCTTTGATGGTTGT | | |
| | TACCATGACAGCGAGATTGACATGGACCAGACTTCCCTCTCTCTAAACTCAGAAGACT | | |
| | TCATGAGAAGAAAATCGATATGCAGCCCCAGCACCGATTCATCTCGGATAAAGAGAAG | | |
| | AAAATCCCTAGGAGGACATGTTGGTAGAATCATTCTGGAAAACAACCATGTCATTGAC | | |
| | ACCTATTCTAGGATTTTATTCCCCATTGTGTATATCTTTATTTAATTT | | |
| | ORF Start: ATG at 7 | | ORF Stop: TAA at 1435 |
| | SEQ ID NO:10 | 476 aa | MW at 55285.2 kD |

| | |
|---|---|
| NOV3a, | MVLAFQLVSFTYIWIILKPNVCAASNIKMTHQRCSSSMKQTWKQETRMKKDDSTKARP |
| CG58518-01 Protein Sequence | |
| | QKYEQLLHIEDNDFAMRPGFGGEYYPLKIGSPVPVGIDVHVESIDSISETNMVSFFMG |
| | YDFIMTFYLRHYWKDERLSFPSTANKSMTFDHRLTRKIWVPDIFFVHSKRSFIHDTTM |
| | ENIMLRVHPDGNVLLSLRRITVSAMCFMDFSRFPLDTQNCSLELESAYNEDDLMLYWK |
| | HGNKSLNTEEHMSLSQFFIEDFSASSGLAFYSSTGWYNRLFINFVLRRHVFFFVLQTY |
| | FPAILMVMLSWVSFWIDRRAVPARVSLGGITTVLTMSTIITAVSASMPQVSYLKAVDV |
| | YLWVSSLFVFLSVIEYAAVNYLTTVEERKQFKKTGKVQISRMYNIDAVQAMAFDGCYH |
| | DSEIDMDQTSLSLNSEDFMRRKSICSPSTDSSRIKRRKSLGGHVGRIILENNHVIDTY |
| | SRILFPIVYIFI |

Further analysis of the NOV3a protein yielded the following properties shown in Table 3B.

TABLE 3B
Protein Sequence Properties NOV3a

| | |
|---|---|
| PSort | 0.6850 probability located in endoplasmic reticulum |
| analysis: | (membrane); 0.6400 probability located in plasma membrane; |
| | 0.4600 probability located in Golgi body; 0.2400 probability |
| | located in nucleus |
| SignalP | Likely cleavage site between residues 25 and 26 |
| analysis: | |

A search of the NOV3a protein against the Geneseq database, a proprietary database that contains sequences published in patents and patent publication, yielded several homologous proteins shown in Table 3C.

TABLE 3C
Geneseq Results for NOV3a

| | | | Identities/ | |
|---|---|---|---|---|
| | | NOV3a | Similarites | |
| | Protein/ | Residues/ | for the | |
| Geneseq | Organism/Length | Match | Matched | Expect |
| Identifier | [Patent #, Date] | Residues | Region | Value |
| AAU04467 | Human gamma-amino | 1 . . . 474 | 454/475 | 0.0 |
| | butyric acid (GABA) | 1 . . . 456 | (95%) | |
| | receptor protein #1 - | | 454/475 | |
| | Homo sapiens, 467 aa. | | (95%) | |
| | [WO200153489-A1, | | | |
| | 26 JUL. 2001] | | | |
| AAU04470 | Human gamma-amino | 48 . . . 474 | 408/428 | 0.0 |
| | butyric acid (GABA) | 1 . . . 409 | (95%) | |
| | receptor protein #4 - | | 408/428 | |
| | Homo sapiens, 420 aa. | | (95%) | |
| | [WO200153489-A1, | | | |
| | 26 JUL. 2001] | | | |
| AAU04468 | Human gamma-amino | 1 . . . 393 | 370/394 | 0.0 |
| | butyric acid (GABA) | 1 . . . 377 | (93%) | |
| | receptor protein #2 | | 370/394 | |
| | Homo sapiens, 392 aa. | | (93%) | |
| | [WO200153489-A1, | | | |
| | 26 JUL. 2001] | | | |
| AAU04471 | Human gamma-amino | 48 . . . 393 | 324/347 | e−180 |
| | butyric acid (GABA) | 1 . . . 330 | (93%) | |
| | receptor protein #5 - | | (93%) | |
| | [WO200153489-A1, | | | |
| | 26 JUL. 2001] | | | |
| AAU04469 | Human gamma-amino | 1 . . . 192 | 176/192 | 2e−96 |
| | butyric acid (GABA) | 1 . . . 177 | (91%) | |
| | receptor protein #3 - | | 176/192 | |
| | Homo sapiens, 180 aa. | | (91%) | |
| | [WO200153489-A1, | | | |
| | 26 JUL. 2001] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | Identities/ | |
|---|---|---|---|---|
| Protein | | Residues/ | Similarities for | |
| Accession | Protein/Organism/ | Match | the Matched | Expect |
| Number | Length | Residues | Portion | Value |
| P50573 | Gamma-amino- | 1 . . . 474 | 383/476 (80%) | 0.0 |
| | butyric-acid | 1 . . . 453 | 407/476 (85%) | |
| | receptor rho-3 | | | |
| | subunit precursor | | | |
| | (GABA(A) | | | |
| | receptor) - Rattus | | | |
| | norvegicus (Rat), | | | |
| | 464 aa. | | | |
| Q9YGQ2 | GAMMA-AMINO- | 1 . . . 474 | 293/485 (60%) | e−153 |
| | BUTYRIC-ACID | 4 . . . 459 | 363/485 (74%) | |
| | RECEPTOR RHO-3 | | | |
| | SUBUNIT - Morone | | | |
| | americana | | | |
| | (White perch), | | | |
| | 470 aa. | | | |
| P50572 | Gamma-amino- | 49 . . . 474 | 270/427 (63%) | e−144 |
| | butyric-acid | 58 . . . 463 | 317/427 (74%) | |
| | receptor rho-1 | | | |
| | subunit precursor | | | |
| | (GABA(A) | | | |
| | receptor) - Rattus | | | |
| | norvegicus (Rat), | | | |
| | 474 aa. | | | |
| P56475 | Gamma-amino- | 49 . . . 474 | 270/427 (63%) | e−143 |
| | butyric-acid | 58 . . . 463 | 317/427 (74%) | |
| | receptor rho-1 | | | |
| | subunit precursor | | | |
| | (GABA(A) | | | |
| | receptor) - Mus | | | |
| | musculus (Mouse), | | | |
| | 474 aa. | | | |
| P24046 | Gamma-amino- | 49 . . . 474 | 268/427 (62%) | e−143 |
| | butyric-acid | 57 . . . 462 | 317/427 (73%) | |
| | receptor rho-1 | | | |
| | subunit precursor | | | |
| | (GABA(A) | | | |
| | receptor) - | | | |
| | Homo sapiens | | | |
| | (Human), 473 aa. | | | |

PFam analysis predicts that the NOV3a protein contains the domains shown in the Table 3E.

TABLE 3E
Domain Analysis of NOV3a

| | NOV3a | | |
|---|---|---|---|
| | Match | Identities/Similarities | Expect |
| Pfam Domain | Region | for the Matched Region | Value |
| Neur_chan_LBD: | 88 . . . 282 | 70/250 (28%) | 1.2e−54 |
| domain 1 of 1 | | 165/250 (66%) | |
| Neur_chan_memb: | 289 . . . 475 | 44/292 (15%) | 7.6e−28 |
| domain 1 of 1 | | 141/292(48%) | |

### Example 4

The NOV4 clone was analyzed, and the nucleotide and predicted polypeptide sequences are shown in Table 4A.

TABLE 4A
NOV4 Sequence Analysis

| | SEQ ID NO:11 | 1587 bp | |
|---|---|---|---|
| NOV4a, | GAACAGAAATGAATAAAAGTCGCTGGCAGAGTAGAAGACGACATGGGAGAAGAAGCCA | | |
| CG58516-01 DNA Sequence | | | |
| | CCAGCAGAACCCTTGGTTCAGACTCCGTGATTCTGAAGACAGGTCTGACTCCCGGGCA | | |
| | GCACAGCCCGCTCACGATTCCGGCCACGGTGATGACGAGTCTCCGTCAACCTCGTCTG | | |
| | GCACAGCTGGGACCTCCTCTGTGCCAGAGCTACCTGGGTTTTACTTTGACCCTGAAAA | | |
| | GAAACGCTACTTCCGCTTGCTCCCTGGACATAACAACTGCAACCCCCTGACGAAAGAG | | |
| | AGCATCCGGCAGAAGGAGATGGAGAGCAAGAGACTGCGGCTGCTCCAGGAAGAAGACA | | |
| | GACGGAAAAAGATTGCCAGGATGGGATTTAATGCATCTTCCATGCTACGAAAAAGCCA | | |
| | GCTGGGTTTTCTCAACGTCACCAATTACTGCCATTTAGCCCACGAGCTGCGTCTCAGC | | |
| | TGCATGGAGAGGAAAAAGGTCCAGATTCGAAGCATGGATCCCTCCGCCTTGGCAAGCG | | |
| | ACCGATTTAACCTCATACTGGCAGATACCAACAGTGACCGGCTCTTCACAGTGAACGA | | |
| | TGTTACAGTTGGAGGCTCCAAGTATGGTATCATCAACCTGCAAGTCTGAAGACCCCT | | |
| | ACGCTCAAGGTGTTCATGCCACGAAAACCTCCGATTCTCACCAACCGGAAGGTGAACA | | |
| | CTTCGGTGTGCTGGGCCTCGCTGAATCACTTGGATTCCCACATTCTGCTATGCCTCAT | | |
| | GGGACTCGCAGAGACTCCAGGCTGTGCCACCCTGCTCCCAGCATCACTGTTCGTCAAT | | |
| | AGTCCCCACCCAGGAATAGACCGGCCTGGCATGCTCTGCAGTTTCCGGATCCCTGGGG | | |
| | GTGCCTGGTCCTGTGCCTGGTCCCTGAATATCCAAGCAAATAACTGCTTCAGTACAGG | | |
| | CTTGTCTCGGCGGGTCCTGTTGACCAACGTGGTGACGGGACACCGGCAGTCCTTTGGG | | |
| | ACCAACAGTGATGTCTTGGCCCAGCAGTTTGCTCTCATGGCTCCTCTGCTGTTTAATG | | |
| | GCTGCCGCTCTGGGGAAATCTTTGCCATTGATCTGCGTTGTGGAAATCAAGGCAAGGG | | |
| | ATGGAAGGCCACCCGCCTGTTTCATGATTCAGCAGTGACCTCTGTGCGGATCCTCCAA | | |
| | GATGAGCAATACCTGATGGCTTCAGACATGGCTGGAAAGATCAAGCTGTGGGACCTGA | | |
| | GGACCACGAAGTGCGTAAGGCAGTACGAAGGCCACGTGAATGAGTACGCCTACCTGCC | | |
| | CCTGCATGTGCACGAGGAAGAAGGAATCCTGGTGGCAGTGGGCCAGGACTGCTACACG | | |
| | AGAATCTGGAGCCTCCACGATGCCCGCCTACTGAGAACCATACCCTCCCCGTACCCTG | | |
| | CCTCCAAGGCCGACATTCCCAGTGTGGCCTTCTCGTCGCGGCTGGGGGGCTCCCGGGG | | |
| | AGAATCTGGAGCCTCCACGATGCCCGCCTACTGAGAACCATACCCTCCCCGTACCCTG | | |
| | CCTCCAAGGCCGACATTCCCAGTGTGGCCTTCTCGTCGCGGCTGGGGGGCTCCCGGGG | | |
| | GCGCGCCGGGGCTGCTCATGGCTGTCGGGCAGGACCTTTACTGTTACTCCTACAGCTA | | |
| | ATTCTGCAGGGCACAGCCCAGAGCCATGTGGATTTGACTTACGGGAGTAAAGCGTAAC | | |
| | TTTTTACTGCATCTAATGAGG | | |
| | ORF Start: ATG at 9 | | ORF Stop: TAA at 1563 |
| | SEQ ID NO:12 | 518 aa | MW at 57769.3 kD |

| | |
|---|---|
| NOV4a, | MNKSRWQSRRHGRRSHQQNPWFRLRDSEDRSDSRAAQPAHDSGHGDDESPSTSSGTA |
| CG58516-01 Protein Sequence | |
| | GTSSVPELPGFYFDPEKKRYFRLLPGENNCNPLTKESIRQKEMESKRLRLLQEEDRRK |
| | KIARMGFNASSMLRKSQLGFLNVTNYCHLAHELRLSCMERKKVQIRSMDPSALASDRF |
| | NLILADTNSDRLFTVNDVTVGGSKYGIINLQSLKTPTLKVFMPRKPPILTNRKVNTSV |
| | CWASLNHLDSHILICLMGLAETPGCATLLPASLFVNSPHPGIDRPGMLCSFRIPGGAW |
| | SCAWSLNIQANNCFSTGLSRRVLLTNVVTGHRQSFGTNSDVLAQQFALMAPLLFNGCR |
| | SGEIFAIDLRCGNQGKGWKATRLFHDSAVTSVRILQDEQYLMASDMAGKIKLWDLRTT |
| | KCVRQYEGHVNEYAYLPLHVHEEEGILVAVGQDCYTRIWSLHDARLLRTIPSPYPASK |
| | ADIPSVAFSSRLGGSRGRAGAAHGCRAGPLLLLLQLILQGTAQSHVDLTYGSKA |

Further analysis of the NOV4a protein yielded the following properties shown in Table 4B.

TABLE 4B
Protein Sequence Properties NOV4a

| | |
|---|---|
| Psort | 0.9600 probability located in nucleus; 0.4776 probability |
| analysis: | located in mitochondrial matrix space; 0.3000 probability |
| | located in microbody (peroxisome); 0.1837 probability located |
| | in mitochondrial inner membrane |
| SignalP | No Known Signal Sequence Predicted |
| analysis: | |

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | Protein/ | Residues/ | Similarities | |
| Geneseq | Organism/Length | Match | for the | Expect |
| Identifier | [Patent #, Date] | Residues | Matched Region | Value |
| ABB11794 | Human secreted | 1 . . . 484 | 470/484 (97%) | 0.0 |
| | protein | 5 . . . 485 | 471/484 (97%) | |
| | homologue SEQ | | | |
| | ID NO:2164— | | | |
| | Homo sapiens, | | | |
| | 500 aa. | | | |
| | [WO200157188- | | | |
| | A2, Aug. 9, | | | |
| | 2001] | | | |
| AAM79804 | Human protein | 1 . . . 484 | 470/484 (97%) | 0.0 |
| | SEQ ID NO | 5 . . . 485 | 471/484 (97%) | |
| | 3450—Homo | | | |
| | sapiens, 500 aa. | | | |
| | [WO200157190- | | | |
| | A2, Aug. 9, | | | |
| | 2001] | | | |
| AAM41122 | Human poly- | 1 . . . 484 | 470/484 (97%) | 0.0 |
| | peptide SEQ ID | 5 . . . 485 | 471/484 (97%) | |
| | NO 6053—Homo | | | |
| | sapiens, 500 aa. | | | |
| | [WO200153312- | | | |
| | A1, Jul. 26, | | | |
| | 2001] | | | |
| AAG67256 | Amino acid | 1 . . . 484 | 459/484 (94%) | 0.0 |
| | sequence of a | 1 . . . 474 | 462/484 (94%) | |
| | human liver- | | | |
| | associated gene— | | | |
| | Homo sapiens, | | | |
| | 489 aa. | | | |
| | [WO200109318- | | | |
| | A1, Feb. 8, | | | |
| | 2001] | | | |
| AAB94587 | Human protein | 1 . . . 484 | 459/484 (94%) | 0.0 |
| | sequence SEQ ID | 1 . . . 474 | 462/484 (94%) | |
| | NO:15389— | | | |
| | Homo sapiens, | | | |
| | 489 aa. | | | |
| | [EP1074617-A2, | | | |
| | Feb. 7, 2001] | | | |

In a BLAST search of public sequence databases, the NOV4a protein was found to have homology to the proteins shown in the BLASTP data in Table 4D.

TABLE 4D
Public BLASTP Results for NOV4a

| | | | Identities/ | |
|---|---|---|---|---|
| | | NOV4a | Similarities | |
| Protein | | Residues/ | for the | |
| Accession | Protein/ | Match | Matched | Expect |
| Number | Organism/Length | Residues | Portion | Value |
| AAH18979 | HYPOTHETICAL | 1 . . . 484 | 470/484 | 0.0 |
| | 55.7 KDA | | (97%) | |
| | PROTEIN— | 1 . . . 480 | 471/484 | |
| | Homo sapiens | | (97%) | |
| | (Human), 495 aa. | | | |
| Q96K22 | CDNA FLJ14839 | 1 . . . 484 | 459/484 | 0.0 |
| | FIS, CLONE | | (94%) | |
| | OVARC1001791— | 1 . . . 474 | 462/484 | |
| | Homo sapiens | | (94%) | |
| | (Human), 489 aa. | | | |
| Q9Y4P5 | HYPOTHETICAL | 5 . . . 435 | 420/431 | 0.0 |
| | 48.5 KDA | | (97%) | |
| | PROTEIN— | 2 . . . 428 | 421/431 | |
| | Homo sapiens | | (97%) | |
| | (Human), 430 aa | | | |
| | (fragment). | | | |
| Q99LF7 | HYPOTHETICAL | 1 . . . 484 | 378/485 | 0.0 |
| | 58.1 KDA | | (77%) | |
| | PROTEIN— | 1 . . . 481 | 423/485 | |
| | Mus musculus | | (86%) | |
| | (Mouse), 519 aa. | | | |
| Q9UF10 | HYPOTHETICAL | 269 . . . 483 | 175/215 | 4e−99 |
| | 26.0 KDA | | (81%) | |
| | PROTEIN— | 4 . . . 217 | 193/215 | |
| | Homo sapiens | | (89%) | |
| | (Human), 234 aa | | | |
| | (fragment). | | | |

PFam analysis predicts that the NOV4a protein contains the domains shown in the Table 4E.

TABLE 4E
Domain Analysis of NOV4a

| | | Identities/ | |
|---|---|---|---|
| | NOV4a | Similarities for | |
| Pfam Domain | Match Region | the Matched Region | Expect Value |
| WD40: domain | 281 . . . 316 | 2/37 (5%) | 5.8e+02 |
| 1 of 3 | | 26/37 (70%) | |
| WD40: domain | 367 . . . 402 | 10/37 (27%) | 6.1 |
| 2 of 3 | | 27/37 (73%) | |
| WD40: domain | 408 . . . 446 | 10/39 (26%) | 13 |
| 3 of 3 | | 23/39 (59%) | |

### Example 5

The NOV5 clone was analyzed, and the nucleotide and predicted polypeptide sequences are shown in Table 5A.

TABLE 5A
NOV5 Sequence Analysis

| | SEQ ID NO:13 | 1081 bp | |
|---|---|---|---|
| NOV5a, | AGGATGGCCCAGAAGGAGAACAGTTATCCCTGGCCCTATGGCAAGCAGACGGCTCCAG | | |
| CG58473-01 DNA Sequence | | | |
| | CCGGCCTGAGTACCCTGCTCCCGCGAGTCCTCCCGAGGATCCCCACCGAAGCTGCGCG | | |
| | TGAGCTCCCGAGCTGCGCAGACCCACAGCCCGCAGCGGCCCCTGGCCATGAGGTGGTA | | |
| | GAGAACAGTTGTGGGAAGCGCAGCATCTTAACGCGGCCCTTCCTGGTCGACGACCTTG | | |
| | AGACTGGGCGTCCCCTGGGCAAAGACAAGTTTGTACATGTGTACTTGGCTCGAAAGAA | | |
| | GACAAGCCATTTCATCGTGGCCCTCAAGGCCTTCAAGTCTCAGATAGAGGAGGGCGTG | | |
| | GAGCACCAGATGCGCAGGCAGATGGAAATCCAGGCCCCCTTTCAGCATCCCAACATAT | | |
| | TGAGTCTCTACAACTATTTTTATGACCTGAGAAAAATCTACTGGATTCTAGAGTACGC | | |
| | CCCCGCCACCCCTACCCCCGAGGAGCTGTACCAGGAGCTGCGAAAGAGCCGCACCTTT | | |
| | GACAAGAAGCCAACAGCCACCATCACGGGGGAGGTGGCAGATGCTCTGATGTACTGCC | | |
| | GAGCACCAGATGCGCAGGCAGATGGAAATCCAGGCCCCCTTTCAGCATCCCAACATAT | | |
| | TGAGTCTCTACAACTATTTTTATGACCTGAGAAAAATCTACTGGATTCTAGAGTACGC | | |
| | CCCCGCCACCCCTACCCCCGAGGAGCTGTACCAGGAGCTGCGAAAGAGCCGCACCTTT | | |
| | GACAAGAAGCCAACAGCCACCATCACGGGGGAGGTGGCAGATGCTCTGATGTACTGCC | | |
| | ACGGGAAGAAGGTGACTCCCAGAGACATGAAGCCAGATAATCTACTCTCAGGGCTTGA | | |
| | GGGCGAGCTGAAAGTTGCCGACTTCGGCTGCCCTGTGCACGCCCCCTCACTGAGGAGG | | |
| | AAGACAAGACAAATGTGTGGCACCCTGGACTACCTGTCCCCAGAGACAATTGAGGGGC | | |
| | GCGCGCACACCGAGAAGGTGGATTTGTGGTACATCGGAGCACTCGGCTATGAGCCGCT | | |
| | GGTGGGGAACCCCACACACAATGAGGCCTATGGGCGAATCGTCAAGGTGGCCCTAAAA | | |
| | TTCCCCCTTCTGTGCCCAGGAGAGCCCCAGGACCTCATCTCCAAGCTGCTTAGGCATA | | |
| | ACCCCTCAGAACGGCTGCCCCTGGCCCAGGTCTCAGCCCACCCTGGGATCCTGGCCCA | | |
| | TTCTCGGAGGGTTTTGCCTCCCTCTGCCCATCAGTCTGTCCCCTGGTGGTCCCTGACA | | |
| | TTCACTCGGGGGCGTCTGTGTTTGTAAGTCTGCATAT | | |
| | ORF Start: ATG at 4 | | ORF Stop: TAA at 1069 |
| | SEQ ID NO:14 | 355 aa | MW at 40012.7 kD |

| | |
|---|---|
| NOV5a, | MAQKENSYPWPYGKQTAPAGLSTLLPRVLPRIPTEAARELPSCADPQPAAAPGHEVVE |
| CG58473-01 Protein Sequence | |
| | NSCGKRSILTRPFLVDDLETGRPLGKDKFVHVYLARKKTSHFIVALKAFKSQIEEGVE |
| | HQMRRQMEIQAPFQHPNILSLYNYFYDLRKIYWILEYAPATPTPEELYQELRKSRTFD |
| | KKPTATITGEVADALMYCHGKKVTPRDMKPDNTLLSGLEGELKVADFGCPVHAPSLRRK |
| | TRQMCGTLDYLSPETIEGRAHTEKVDLWYIGALGYEPLVGNPTHNEAYGRIVKVALKF |
| | PLLCPGEPQDLISKLLRHNPSERLPLAQVSAHPGILAHSRRVLPPSAHQSVPWWSLTF |
| | TRGRLCL |

Further analysis of the NOV5a protein yielded the following properties shown in Table 5B1

TABLE 5B
Protein Sequence Properties NOV5a

| | |
|---|---|
| Psort | 0.4500 probability located in cytoplasm; 0.3000 probability |
| analysis: | located in microbody (peroxisome); 0.1897 probability located |
| | in lysosome (lumen); 0.1000 probability located in |
| | mitochondrial matrix space |
| SignalP | No Known Signal Sequence Predicted |
| analysis: | |

A search of the NOV5a protein against the Geneseq database, a proprietary database that contains sequences published in patents and patent publication, yielded several homologous proteins shown in Table 5C.

TABLE 5C
Geneseq Results for NOV5a

| | | NOV5a | Identities/ | |
|---|---|---|---|---|
| | Protein/ | Residues/ | Similarities | |
| Genseq | Organism/Length | Match | for the | Expect |
| Identifier | [Patent #, Date] | Residues | Matched Region | Value |
| AAG67615 | Amino acid | 1 . . . 341 | 247/349 (70%) | e−129 |
| | sequence of | 1 . . . 343 | 274/349 (77%) | |
| | a human | | | |
| | [WO200109316- | | | |
| | A1, Feb. 8, 2001] | | | |
| AAG67436 | Amino acid | 1 . . . 341 | 247/349 (70%) | e−129 |
| | sequence of a | 1 . . . 343 | 274/349 (77%) | |
| | human poly- | | | |
| | peptide—Homo | | | |
| | sapiens, 344 aa. | | | |
| | [WO200109345- | | | |
| | A1, Feb. 8, 2001] | | | |
| AAY22475 | Human AUR1 | 1 . . . 341 | 247/349 (70%) | e−129 |
| | protein | 1 . . . 343 | 274/349 (77%) | |
| | sequence—Homo | | | |
| | sapiens, 344 aa. | | | |
| | [WO9937788-A2, | | | |
| | Jul. 29, 1999] | | | |
| AAW18083 | Human Aurora- | 1 . . . 341 | 247/349 (70%) | e−129 |
| | 1—Homo | 1 . . . 343 | 274/349 (77%) | |
| | sapiens, 344 aa. | | | |
| | [WO9722702-A1, | | | |
| | Jun. 26, 1997] | | | |
| AAY27052 | Human protein | 1 . . . 341 | 246/352 (69%) | e−127 |
| | kinase (HPKM)-1 | 1 . . . 346 | 274/352 (76%) | |
| | (clone ID | | | |
| | 2940)—Homo | | | |
| | sapiens, 347 aa. | | | |
| | [WO9938981-A2, | | | |
| | Aug. 5, 1999] | | | |

In a BLAST search of public sequence databases, the NOV5a protein was found to have homology to the proteins shown in the BLASTP data in Table 5D.

TABLE 5D
Public BLASTP Results for NOV5a

| | | NOV5a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| O60446 | AURORA- | 1 . . . 341 | 247/349 (70%) | e−128 |
| | RELATED | 1 . . . 343 | 274/349 (77%) | |
| | KINASE 2 | | | |
| | (SERINE/ | | | |
| | THREONINE | | | |
| | KINASE 12)— | | | |
| | Homo sapiens | | | |
| | (Human), 344 aa. | | | |
| Q96GD4 | UNKNOWN | 1 . . . 341 | 247/349 (70%) | e−128 |
| | (PROTEIN FOR | 1 . . . 343 | 274/349 (77%) | |
| | MGC:11031)— | | | |
| | Homo sapiens | | | |
| | (Human), 344 aa. | | | |
| Q96DV5 | UNKNOWN | 1 . . . 341 | 247/350 (70%) | e−126 |
| | (PROTEIN FOR | 1 . . . 344 | 274/350 (77%) | |
| | MGC:4243)— | | | |
| | Homo sapiens | | | |
| | (Human), 345 aa. | | | |
| Q9UQ46 | AIK2—Homo | 1 . . . 341 | 245/348 (70%) | e−126 |
| | sapiens (Human), | 1 . . . 342 | 272/348 (77%) | |
| | 343 aa. | | | |
| O14630 | PROTEIN | 1 . . . 341 | 245/352 (69%) | e−125 |
| | KINASE—Homo | 1 . . . 346 | 272/352 (76%) | |
| | sapiens (Human), | | | |
| | 347 aa. | | | |

PFam analysis predicts that the NOV5a protein contains the domains shown in the Table SE.

TABLE 5E
Domain Analysis of NOV5a

| | | Identities/ | |
|---|---|---|---|
| | NOV5a | Similarities for | Expect |
| Pfam Domain | Match Region | the Matched Region | Value |
| Pkinase: domain | 76 . . . 325 | 81/293 (28%) | 6.5e−36 |
| 1 of 1 | | 184/293 (63%) | |

### Example 6

The NOV6 clone was analyzed, and the nucleotide and predicted polypeptide sequences are shown in Table 6A.

TABLE 6A
NOV6 Sequence Analysis

| | SEQ ID NO:15 | 1524 bp | |
|---|---|---|---|
| NOV6a, | AGCATTATGAACACTAATGACCTTAAACTCAGGTTGTCCAAAGCTGAGCAAGAACACC | | |
| CG58470-01 DNA Sequence | | | |
| | CACTACGTTTCTGGAATGAGCTTGAAGAAGCCCGACAGGTAGAACTTTATGCAGAGCT | | |
| | CCAGGCCATCGACTTTCAGGAACTGAACTTCTTTTTCCAAAAGGCCATTGAAGGATTT | | |
| | AACCAGTCCTCTCATCAAGAAAAGGTGGATGCGGGAATGGAACCTGTCCCTCGAGAAG | | |
| | TACTGGGCAGTGCTGCAGGGAAGCTAGATCAGCTCCAGGCCTGGGAAAGCAAAGTTTT | | |
| | CCAGATTTCTGAGAACAAAGTCACAGTTGTTCTAGCTGGTGGGCAGGGGACTAGACTC | | |
| | GTTGCATATCCAAAGGGGATGTATGATGTTGGTTTGCCATCCCATAAGACACTTTTTC | | |
| | AGATTCAAGCAGAGCATATCCTGAAGCTACAACAGTTAGCTGAAAAATATTATGGCAA | | |
| | CAAATGCATTATTCCATATTACGTCATGACCAGCGAGTTCACTCTGGGGCCCACGGCC | | |
| | GAGTTCTTCAGGGAGCACAACTTCTTCCACCTGGACCCCGCCAACGTGGTCATGTTTG | | |
| | AGCAGCGCCTGCTGCCTGCTGTGACCTTTGATGGCAAGGTTATCCTGGAGCGGAAAGA | | |
| | CAAAGTTGCCATGGCCCCAGACGGCAACGGGGGCCTCTACTGCGCGCTGGAGGACCAC | | |
| | AAGATCCTGGAGGACATGGAGCGCCGGGGAGTGGAGTTTGTGCACGTGTACTGTGTGG | | |
| | ACAACATCCTGGTGCGGCTGGCGGACCCTGTCTTCATCGGCTTCTGTGTGTTGCAGGG | | |
| | CGCAGACTGTGGCGCCAAGGTGGTGGAAAAGGCATACCCCGAGGAGCCCGTGGGCGTG | | |
| | GTGTGCCAGGTGGACGGTGTCCCCCAGGTGGTGGAGTACAGCGAGATCAGTCCTGAGA | | |
| | CCGCACAGCTACGTGTCTCCGACGGGAGCCTGCTGTACAATGCAGGCAACATCTGCAA | | |
| | CCACTTCTTCACCCGAGGCTTCCTTAAGGCGGTCACCAGGGAGTTTGAGCCTTTGCTG | | |
| | AAGCCACACGTGGCTGTGAAGAAGGTCCCGTATGTGGATGAGGAGGGGAATCTGGTAA | | |
| | AGCCGCTAAAACCGAACGGGATAAAGATGGAGAAGTTTGTGTTTGATGTGTTCCGGTT | | |
| | TGCTAAGAACTTTGCTGCCTTGGAAGTGCTGCGGGAGGAGGAATTTTCCCCACTGAAG | | |
| | AACGCAGAGCCAGCCGACAGGGACAGTCCCCGCACCGCTCGCCAGGCCCTGCTCACCC | | |
| | AGCACTACCGGTGGGCTCTGCGGGCCGGGGCCCGCTTCCTGGATGCCCATGGGGCCCG | | |
| | GCTCCCAGAGCTGCCCAGCTTGCCCCCAAATGGAGACCCTCCGGCCATCTGTGAGATA | | |
| | TCGCCCTTGGTGTCTTACTCTGGAGAGGGTTTAGAAGTGTACCTGCAAGGCCGGGAGT | | |
| | TCCAGTCCCCGCTCATCCTGGATGAAGACCAGGCCAGGGAGCTGGTGAAAAATGGTAT | | |
| | ATGAACCTGATACCAA | | |
| | ORF Start: ATG at 7 | | ORF Stop: TGA at 1510 |
| | SEQ ID NO:16 | 501 aa | MW at 56461.0 kD |

| | |
|---|---|
| NOV6a, | MNTNDLKLRLSKAEQEHPLRFWNELEEARQVELYAELQAIDFQELNFFFQKAIEGFNQ |
| CG58470-01 Protein Sequence | |
| | SSHQEKVDAGMEPVPREVLGSAAGKLDQLQAWESKVFQISENKVTVVLAGGQGTRLVA |
| | YPKGMYDVGLPSHKTLFQIQAEHILKLQQLAEKYYGNKCIIPYYVMTSEFTLGPTAEF |
| | FREHNFFHLDPANVVMFEQRLLPAVTFDGKVILERKDKVAMAPDGNGGLYCALEDHKI |
| | LEDMERRGVEFVHVYCVDNILVRLADPVFIGFCVLQGADCGAKVVEKAYPEEPVGVVC |
| | QVDGVPQVVEYSEISPETAQLRVSDGSLLYNAGNICNHFFTRGFLKAVTREFEPLLKP |
| | HVAVKKVPYVDEEGNLVKPLKPNGIKMEKFVFDVFRFAKNFAALEVLREEEFSPLKNA |
| | EPADRDSPRTARQALLTQHYRWALRAGARFLDAHGARLPELPSLPPNGDPPAICEISP |
| | LVSYSGEGLEVYLQGREFQSPLILDEDQARELVKNGI |

Further analysis of the NOV6a protein yielded the following properties shown in Table 6B.

TABLE 6B
Protein Sequence Properties NOV6a

| | |
|---|---|
| PSort | 0.4500 probability located in cytoplasm; 0.3490 probability |
| analysis: | located in microbody (peroxisome); 0.1000 probability located |
| | in mitochondrial matrix space; 0.1000 probability located |
| | in lysosome (lumen) |
| SignalP | No Known Signal Sequence Predicted |
| analysis: | |

A search of the NOV6a protein against the Geneseq database, a proprietary database that contains sequences published in patents and patent publication, yielded several homologous proteins shown in Table 6C.

TABLE 6C
Geneseq Results for NOV6a

| | | NOV6a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAB56960 | Human prostate | 1 . . . 501 | 353/522 (67%) | 0.0 |
| | cancer antigen | 3 . . . 524 | 413/522 (78%) | |
| | protein sequence | | | |
| | SEQ ID NO: | | | |
| | 1538—Homo | | | |
| | sapiens, 524 aa. | | | |
| | [WO200055174- | | | |
| | A1, Sep. 21, | | | |
| | 2000] | | | |
| AAG32392 | Arabidopsis | 9 . . . 485 | 194/489 (39%) | 3e−84 |
| | thaliana protein | 36 . . . 497 | 275/489 (55%) | |
| | fragment SEQ ID | | | |
| | NO: 39067— | | | |
| | Arabidopsis | | | |
| | thaliana, 502 aa. | | | |
| | [EP1033405-A2, | | | |
| | Sep. 6, 2000] | | | |
| AAG40236 | Arabidopsis | 9 . . . 485 | 193/488 (39%) | 3e−82 |
| | thaliana protein | 12 . . . 472 | 272/488 (55%) | |
| | fragment SEQ ID | | | |
| | NO: 49896— | | | |
| | Arabidopsis | | | |
| | thaliana, 477 aa. | | | |
| | [EP1033405-A2, | | | |
| | Sep. 6, 2000] | | | |
| AAG40235 | Arabidopsis | 9 . . . 485 | 193/488 (39%) | 3e−82 |
| | thaliana protein | 35 . . . 495 | 272/488 (55%) | |
| | fragment SEQ ID | | | |
| | NO: 49895— | | | |
| | Arabidopsis | | | |
| | thaliana, 500 aa. | | | |
| | [EP1033405-A2, | | | |
| | Sep. 6, 2000] | | | |
| AAG40234 | Arabidopsis | 9 . . . 485 | 193/488 (39%) | 3e−82 |
| | thaliana protein | 40 . . . 500 | 272/488 (55%) | |
| | fragment SEQ ID | | | |
| | NO: 49894— | | | |
| | Arabidopsis | | | |
| | thaliana, 505 aa. | | | |
| | [EP1033405-A2, | | | |
| | Sep. 6, 2000] | | | |

In a BLAST search of public sequence databases, the NOV6a protein was found to have homology to the proteins shown in the BLASTP data in Table 6D.

TABLE 6D
Public BLASTP Results for NOV6a

| | | NOV6a | Identities/ | |
|---|---|---|---|---|
| Protein | | Residues/ | Similarities for | |
| Accession | Protein/ | Matched | the Matched | Expect |
| Number | Organism/Length | Residues | Portion | Value |
| Q96GM2 | UDP-N- | 1 . . . 501 | 351/505 (69%) | 0.0 |
| | ACTEYL- | 1 . . . 505 | 412/505 (81%) | |
| | GLUCOSAMINE | | | |
| | PYROPHOS- | | | |
| | PHORYLASE | | | |
| | 1—Homo sapiens | | | |
| | (Human), 505 aa. | | | |
| Q16222 | UDP-N-acetyl- | 1 . . . 501 | 352/522 (67%) | 0.0 |
| | hexosamine pyro- | 1 . . . 522 | 412/522 (78%) | |
| | phosphorylase | | | |
| | (Antigen X) | | | |
| | (AGX) (Sperm- | | | |
| | associated | | | |
| | antigen 2) | | | |
| | [Includes: UDP- | | | |
| | N-acetylgalactos- | | | |
| | amine pyro- | | | |
| | phosphorylase | | | |
| | (EC 2.7.7.-) | | | |
| | (AGX-1); UDP- | | | |
| | N-acetylglucos- | | | |
| | amine pyro- | | | |
| | phosphorylase | | | |
| | (EC 2.7.7.23) | | | |
| | (AGX-2)]— | | | |
| | Homo sapiens | | | |
| | (Human), 522 aa. | | | |
| Q91YN5 | HYPO- | 1 . . . 501 | 342/522 (65%) | 0.0 |
| | THETICAL | 1 . . . 522 | 407/522 (77%) | |
| | 58.6 KDA | | | |
| | PROTEIN— | | | |
| | Mus musculus | | | |
| | (Mouse), 522 aa. | | | |
| AAH17547 | HYPO- | 1 . . . 501 | 341/521 (65%) | 0.0 |
| | THETICAL | 1 . . . 521 | 407/521 (77%) | |
| | 58.5 KDA | | | |
| | PROTEIN— | | | |
| | Mus musculus | | | |
| | (Mouse), 521 aa. | | | |
| Q9Y0Z0 | BCDNA: | 6 . . . 492 | 236/491 (48%) | e−124 |
| | LD24639 | 44 . . . 513 | 330/491 (67%) | |
| | PROTEIN— | | | |
| | Drosophila | | | |
| | melanogaster | | | |
| | (Fruit fly), | | | |
| | 520 aa. | | | |

PFam analysis predicts that the NOV6a protein contains the domains shown in the Table 6E.

TABLE 6E
Domain Analysis of NOV6a

| | | Identities/ | |
|---|---|---|---|
| | NOV6a | Similarities for | Expect |
| Pfam Domain | Match Region | the Matched Region | Value |
| UDPGP: domain | 40 . . . 434 | 108/428 (25%) | 8.4e−111 |
| 1 of 1 | | 324/428 (76%) | |

### Example 7

The NOV7 clone was analyzed, and the nucleotide and predicted polypeptide sequences are shown in Table 7A.

TABLE 7A
NOV7 Sequence Analysis

| | SEQ ID NO:17 | 461 bp | |
|---|---|---|---|
| NOV7a, | ACGCAGAGATGCAGATCTTTGTGAAGACCCTCACGGGCAAGACCATCACCCTTGAGGT | | |
| CG58593-01 DNA Sequence | | | |
| | CAAGCCCACCGACACCATTGAGAATGTCAAAACCAAAATTCAGGACAAGGAGGGTATC | | |
| | CCACCTGACCAGCAGCGTCTGATATTTGCTGGGAAACGGCTGGAGGATGGCCACACTC | | |
| | TCTCAGGCTACAACATCCAGAAAGAGTCCACCCTAAACCTGGTGCTGCGCCTGCGAGG | | |
| | TGGCATTACTGAGCCTTCCCTCCGCCAGCTCGTCCAGAAATACAACTGCGACGAGATG | | |
| | ATCTGCTGCAAGTGCTATGCTTGCCTGCACCCCGGTGCTATCAACTGCCACAAGAAGA | | |
| | AATGCGGCCACACCAACAACCTGTACCCCAGGAAGAAGGTCAAATAAGGCTCTTCCTT | | |
| | CCTTGAAGGGCAGCAGCCTTCTGCCCAGGCCCCATGGCCCTGGGGCCTCAATAAA | | |
| | ORF Start: ATG at 9 | | ORF Stop: TAA at 393 |
| | SEQ ID NO:18 | 128 aa | MW at 14540.9 kD |

| | |
|---|---|
| NOV7a, | MQIFVKTLTGKTITLEVKPTDTIENVKTKIQDKEGIPPDQQRLIFAGKRLEDGHTLSG |
| CG58593-01 Protein Sequence | |
| | YNIQKESTLNLVLRLRGGITEPSLRQLVQKYNCDEMICCKCYACLHPGAINCHKKKCG |
| | HTNNLYPRKKVK |

Further analysis of the NOV7a protein yielded the following properties shown in Table 7B.

TABLE 7B
Protein Sequence Properties NOV7a

| | |
|---|---|
| PSort | 0.9800 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV7a protein against the Geneseq database, a proprietary database that contains sequences published in patents and patent publication, yielded several homologous proteins shown in Table 7C.

TABLE 7C
Geneseq Results for NOV7a

| | | | Identities/ | |
|---|---|---|---|---|
| | | NOV7a | Similarities | |
| | Protein/ | Residues/ | for the | |
| Geneseq | Organism/Length | Match | Matched | Expect |
| Identifier | [Patent #, Date] | Residues | Region | Value |
| AAB52080 | Gene 16 human | 1 . . . 128 | 111/128 | 7e−61 |
| | secreted protein | | (86%) | |
| | homologous amino | 1 . . . 128 | 118/128 | |
| | acid sequence | | (91%) | |
| | #129—Sus scrofa, | | | |
| | 128 aa. | | | |
| | [WO200061596-A1, | | | |
| | Oct. 19, 2000] | | | |
| AAG43861 | Arabidopsis | 1 . . . 128 | 101/128 | 9e−55 |
| | thaliana protein | | (78%) | |
| | fragment SEQ ID | 1 . . . 128 | 113/128 | |
| | NO: 54871— | | (87%) | |
| | Arabidopsis | | | |
| | thaliana, 128 aa. | | | |
| | [EP1033405-A2, | | | |
| | Sep. 6, 2000] | | | |
| AAG36188 | Arabidopsis | 1 . . . 128 | 101/128 | 9e−55 |
| | thaliana protein | | (78%) | |
| | fragment SEQ ID | 122 . . . 249 | 113/128 | |
| | NO: 44314— | | (87%) | |
| | Arabidopsis | | | |
| | thaliana, 249 aa. | | | |
| | [EP1033405-A2, | | | |
| | Sep. 6, 2000] | | | |
| AAG36187 | Arabidopsis | 1 . . . 128 | 101/128 | 9e−55 |
| | thaliana protein | | (78%) | |
| | fragment SEQ ID | 137 . . . 264 | 113/128 | |
| | NO: 44313— | | (87%) | |
| | Arabidopsis | | | |
| | thaliana, 264 aa. | | | |
| | [EP1033405-A2, | | | |
| | Sep. 6, 2000] | | | |
| AAG36186 | Arabidopsis | 1 . . . 128 | 101/128 | 9e−55 |
| | thaliana protein | | (78%) | |
| | fragment SEQ ID | 195 . . . 322 | 113/128 | |
| | NO: 44312— | | (87%) | |
| | Arabidopsis | | | |
| | thaliana, 322 aa. | | | |
| | [EP1033405-A2, | | | |
| | Sep. 6, 2000] | | | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q9BX98 | UBIQUITIN | 1 . . . 128 | 111/128 (86%) | 3e−60 |
| | A-52 RESIDUE | 14 . . . 141 | 118/128 (91%) | |
| | RIBOSOMAL | | | |
| | PROTEIN | | | |
| | FUSION | | | |
| | PRODUCT 1— | | | |
| | Homo sapiens | | | |
| | (Human), 141 aa | | | |
| | (fragment). | | | |
| Q9UPK7 | UBIQUITIN-52 | 1 . . . 128 | 111/128 (86%) | 3e−60 |
| | AMINO ACID | 1 . . . 128 | 118/128 (91%) | |
| | FUSION | | | |
| | PROTEIN— | | | |
| | Homo sapiens | | | |
| | (Human), 128 aa. | | | |
| Q9PT09 | UBIQUITIN— | 1 . . . 128 | 110/128 (85%) | 6e−60 |
| | Oncorhynchus | 1 . . . 128 | 118/128 (91%) | |
| | mykiss (Rainbow | | | |
| | trout) (Salmo | | | |
| | gairdneri), | | | |
| | 128 aa. | | | |
| O42388 | UBIQUITIN- | 1 . . . 128 | 110/128 (85%) | 7e−60 |
| | RIBOSOMAL | 1 . . . 128 | 117/128 (90%) | |
| | PROTEIN | | | |
| | FUSION | | | |
| | PROTEIN— | | | |
| | Gallus gallus | | | |
| | (Chicken), | | | |
| | 128 aa. | | | |
| Q9XSV1 | UBIQUITIN- | 1 . . . 128 | 110/128 (85%) | 1e−59 |
| | RIBOSOMAL | 1 . . . 128 | 117/128 (90%) | |
| | PROTEIN L40 | | | |
| | FUSION | | | |
| | PROTEIN— | | | |
| | Canis | | | |
| | familiaris | | | |
| | (Dog), 128 aa. | | | |

PFam analysis predicts that the NOV7a protein contains the domains shown in the Table 7E.

TABLE 7E
Domain Analysis of NOV7a

| | | Identities/ | |
|---|---|---|---|
| | NOV7a | Similarities for | Expect |
| Pfam Domain | Match Region | the Matched Region | Value |
| ubiquitin: domain | 1 . . . 74 | 54/83 (65%) | 1.9e−38 |
| 1 of 1 | | 72/83 (87%) | |
| Ribosomal_L40e: | 77 . . . 128 | 30/52 (58%) | 7.3e−20 |
| domain 1 of 1 | | 42/52 (81%) | |

### Example 8

The NOV8 clone was analyzed, and the nucleotide and predicted polypeptide sequences are shown in Table 8A.

TABLE 8A
NOV8 Sequence Analysis

| | SEQ ID NO:19 | 2296 bp | |
|---|---|---|---|
| NOV8a, | CGGCGGCGGCGGCAGTAGAAATGATGGAAGAATTGCATAGCCTGGACCCACGACGGCA | | |
| CG57871-01 DNA Sequence | | | |
| | GAAATTATTGGAGGCCAGGTTTACTGGAGTAGGTGTTAGTAAGGGACCACTTAATAGT | | |
| | GAGTCTTCCAACCAGAGCTTGTGCAGCGTCGGATCCTTGAGTGATAAAGAAGTAGAGA | | |
| | CTCCCAAGAAAAAGCAGAATGACCAGCGAAATCGGAAAAGAAAAGCTGAACCATATGA | | |
| | AAGTAGCCAAGGGAAAGGCACTCCTAGGGGACATAAAATTAGTGATTACTTTGAGTTT | | |
| | GCTGGGGGAAGCGGGCCGGGAACCAGCCCTGGCAGAAGTGTTCCACCAGTTGCACGAT | | |
| | CCTCACTGCAACATTCTTTATCCAATCCCTTACCGCGACGAGTAGAACAGCCCCTCTA | | |
| | TGGTTTAGATGGCAGTGCTGCAAAGGAGGCAACGGAGGAGCAGTCTGCTCTGCCAACC | | |
| | CTCATGTCAGTGATGCTAGCAAAACCTCGGCTTGACACAGAGCAGCTGGCGCAAAGGG | | |
| | GAGCTGGCCTCTGCTTCACTTTTGTTTCAGCTCAGCAAAACAGTCCCTCATCTACGGG | | |
| | ATCTGGCAACACAGAGCATTCCTGCAGCTCCCAAAAACAGATCTCCATCCAGCACAGA | | |
| | CAGACCCAGTCCGACCTCACAATAGAAAAAATATCTGCACTAGAAAACAGTAAGAATT | | |
| | CTGACTTAGAGAAGAAGGAGGGAAGAATAGATGATTTATTAAGAGCCATCTGTGATTT | | |
| | GAGACGGCAGATTGATGAACAGCAAAAGATGCTAGAGAAATACAAGGAACGATTAAAT | | |
| | AGATGTGTGACAATGAGCAAGAAACTCCTTATAGAAAAGTCAAAACAAGAGAAGATGG | | |
| | CGTGTAGAGATAAGAGCATGCAAGACCGCTTGAGACTGGGCCACTTTACTACGTCTGA | | |
| | CCACGGAGCCAAATTTACTGAGCAGTGGACAGATGGTTATGCTTTTCAGAATCTTATC | | |
| | AAGCAACAGGAAAGGATAAATTCACAGAGGGAAGAGATAGAAAGACAACGGAAAATGT | | |
| | TAGCAAAGCGGAAACCTCCTGCCATGGGTCAGGCCCCTCCTGCAACCAATGAGCAGAA | | |
| | ACAGTGGAAAAGCAAGACCAATGGAGCTGAAAATGAAACGTTAACGTTAAAAGAATAC | | |
| | CATGAACAAGAAGAAATCTTCAAACTCAGATTAGGTCATCTTAAAAAGGAGGAAGCAG | | |
| | AGATCCAGGCAGAGCTGGAGAGGCTAGAAAGGGTTAGAAAACTACATATCAGGGAAGT | | |
| | AAAAAGGATACATAATGAAGATAATTCACAATTTAAATATCATCCAACGCTAAATGAC | | |
| | AGATATTTGTTGTTACATCTTTTGGGTAGAGGAGGTTTCAGTGAAGTTTACAAGGCAT | | |
| | TTGATCTAACAGAGCAAAGATACGTAGCTGTGAAAATTCACCAGTTAAATAAAAACTG | | |
| | GAGAGATGAGAAAAAGGAGAATTACCACAAGCATGCATGTAGGGAATACCGGATTCAT | | |
| | AAAGAGCTGGACCATCCCAGAATAGTTAAGCTGTATGATTACTTTTCACTGGATACTG | | |
| | ACTCGTTTTGTACAGTATTAGAATACTGTGAGGGAAATGATCTGGACTTCTACCTGAA | | |
| | ACAGCACAAATTAATGTCAGAGAAAGAGGCCCGGTCCATTATCATGCAGATTGTGAAT | | |
| | GCTTTAAAGTACTTAAATGAAATAAAACCTCCCATCATACACTATGACCTCAAACCAG | | |
| | GTAATATTCTTTTAGAAAATGGTACAGCGTGTGGAGAGATAAAAATTACAGATTTTGG | | |
| | TCTTTCGAAGATCATGGATGATGATAGCTACAATTCAGTGGATGGCATGGAGCTAACA | | |
| | TCACAAGGTGCTGGTACTTATTGGTATTTACCACCAGAGTGTTTTGTGGTTGGGAAAG | | |
| | AACCACCAAAGATCTCAAATAAAGTTGATGTGTGGTCGGTGGGTGTGATCTTCTATCA | | |
| | GTGTCTTTATGGAAGGAAGCCTTTTGGCCATAACCAGTCTCAGCAAGACATCCTACAA | | |
| | GAGAATACGATTCTTAAAGCTACTGAAGTGCAGTTCCCGCCAAAGCCGGTAGTAACAC | | |
| | CTGAAGCAAAGGCGTTGATTCGACGATGCTTGGCCTACCGAAAGGAGGACCGCATTGA | | |
| | TGTCCAGCAGCTGGCCTGTGATCCCTACTTGTTGCCTCACATCCGAAAGTCAGTCTCT | | |
| | ACGAGTAGCCCTGCTGGAGCTGCTATTGCATCAACCTCTGGGGCGTCCAATAACAGTT | | |
| | CTTCTAATTGAGACTGACTCCAAGGCCACAAACT | | |
| | ORF Start: ATG at 24 | | ORF Stop: TGA at 2271 |
| | SEQ ID NO:20 | 749 aa | MW at 8545.8 kD |

| | |
|---|---|
| NOV8a, | MEELHSLDPRRQKLLEARFTGVGVSKGPLNSESSNQSLCSVGSLSDKEVETPKKKQND |
| CG57871-01 Protein Sequence | |
| | QRNRKRKAEPYESSQGKGTPRGHKISDYFEFAGGSGPGTSPGRSVPPVARSSLQHSLS |
| | NPLPRRVEQPLYGLDGSAAKEATEEQSALPTLMSVMLAKPRLDTEQLAQRGAGLCFTF |
| | VSAQQNSPSSTGSGNTEHSCSSQKQISIQHRQTQSDLTIEKISALENSKNSDLEKKEG |
| | RIDDLLRAICDLRRQIDEQQKMLEKYKERLNRCVTMSKKLLIEKSKQEKMACRDKSMQ |
| | DRLRLGHFTTSDHGAKFTEQWTDGYAFQNLIKQQERINSQREEIERQRKMLAKRKPPA |
| | MGQAPPATNEQKQWKSKTNGAENETLTLKEYHEQEEIFKLRLGHLKKEEAEIQAELER |
| | LERVRKLHIREVKRIHNEDNSQFKYHPTLNDRYLLLHLLGRGGFSEVYKAFDLTEQRY |
| | VAVKIHQLNKNWRDEKKENYHKHACREYRIHKELDHPRIVKLYDYFSLDTDSFCTVLE |
| | YCEGNDLDFYLKQHKLMSEKEARSIIMQIVNALKYLNEIKPPIIHYDLKPGNILLENG |
| | TACGEIKITDFGLSKIMDDDSYNSVDGMELTSQGAGTYWYLPPECFVVGKEPPKISNK |
| | VDVWSVGVIFYQCLYGRKPFGHNQSQQDILQENTILKATEVQFPPKPVVTPEAKALIR |
| | RCLAYRKEDRIDVQQLACDPYLLPHIRKSVSTSSPAGAAIASTSGASNNSSSN |

Further analysis of the NOV8a protein yielded the following properties shown in Table 8B.

TABLE 8B
Protein Sequence Properties NOV8a

| | |
|---|---|
| PSort | 0.9600 probability located in nucleus; 0.1000 probability |
| analysis: | located in mitochondrial matrix space; 0.1000 probability |
| | located in lysosome (lumen); 0.0000 probability located |
| | in endoplasmic reticulum (membrane) |
| SignalP | No Known Signal Sequence Predicted |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | | |
|---|---|---|---|---|---|
| | Protein/ | Residues/ | Similarities for | | |
| Geneseq | Organism/Length | Match | the Matched | | Expect |
| Identifier | [Patent #, Date] | Residues | Region | | Value |
| AAM39278 | Human polypeptide SEQ ID NO | 1 . . . 749 | 703/749 | (93%) | 0.0 |
| | 2423 - Homo sapiens, 718 aa. | 2 . . . 718 | 707/749 | (93%) | |
| | [WO200153312-A1, 26 JUL | | | | |
| | 2001] | | | | |
| AAM41064 | Human polypeptide SEQ ID NO | 1 . . . 749 | 695/750 | (92%) | 0.0 |
| | 5995 - Homo sapiens, 809 aa. | 92 . . . 809 | 701/750 | (92%) | |
| | [WO200153312-A1, 26 JUL | | | | |
| | 2001] | | | | |
| AAR76062 | Protein kinase PKU beta - Homo | 210 . . . 749 | 525/540 | (97%) | 0.0 |
| | sapiens, 540 aa. [JP07132093-A, | 1 . . . 540 | 527/540 | (97%) | |
| | 23 MAY 1995] | | | | |
| AAR76061 | Protein kinase PKU alpha - Homo | 1 . . . 744 | 537/794 | (67%) | 0.0 |
| | sapiens, 787 aa. [JP07132093-A, | 49 . . . 783 | 592/794 | (73%) | |
| | 23 MAY 1995] | | | | |
| ABB20910 | Protein #2909 encoded by probe | 346 . . . 749 | 404/404 | (100%) | 0.0 |
| | expression - Homo sapiens, 404 | 1 . . . 404 | 404/404 | (100%) | |
| | aa. [WO200157274-A2, 09 AUG | | | | |
| | 2001] | | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UK17 | TOUSLED-LIKE KINASE 2 - | 1 . . . 749 | 731/749 (97%) | 0.0 |
| | Homo sapiens (Human), 749 aa. | 1 . . . 749 | 736/749 (97%) | |
| O55047 | TOUSLED-LIKE KINASE - Mus | 1 . . . 749 | 699/749 (93%) | 0.0 |
| | musculus (Mouse), 717 aa. | 1 . . . 717 | 705/749 (93%) | |
| Q9Y4F7 | PKU-ALPHA - Homo sapiens | 1 . . . 749 | 700/749 (93%) | 0.0 |
| | (Human), 719 aa (fragment). | 3 . . . 719 | 705/749 (93%) | |
| Q9D5Y5 | TOUSLED-LIKE KINASE 2 | 1 . . . 656 | 629/656 (95%) | 0.0 |
| | (ARABIDOPSIS) - Mus musculus | 1 . . . 656 | 640/656 (96%) | |
| | (Mouse), 696 aa. | | | |
| Q90ZY7 | PKU-ALPHA PROTEIN KINASE - | 1 . . . 749 | 580/753 (77%) | 0.0 |
| | Brachydanio rerio (Zebrafish) | 2 . . . 697 | 626/753 (83%) | |
| | (Zebra danio), 697 aa. | | | |

PFam analysis predicts that the NOV8a protein contains the domains shown in the Table 8E.

TABLE 8E
Domain Analysis of NOV8a

| | | Identities/ | | |
|---|---|---|---|---|
| | | Similarities | | |
| Pfam Domain | NOV8a Match Region | for the Matched Region | | Expect Value |
| A2M: domain 1 of 1 | 501 . . . 523 | 10/23 | (43%) | 4.6 |
| | | 20/23 | (87%) | |
| Pkinase: domain 1 of | 439 . . . 718 | 96/316 | (30%) | 5.4e−70 |
| 1 | | 213/316 | (67%) | |

### Example 9

The NOV9 clone was analyzed, and the nucleotide and predicted polypeptide sequences are shown in Table 9A.

TABLE 9A
NOV9 Sequence Analysis

| | SEQ ID NO:21 | 2060 bp | |
|---|---|---|---|
| NOV9a, | GTTTTCATAGATAACCATGCAACATCCCATATGAATGGGCATGTTACAGAGGAATCA | | |
| CG58590-01 DNA Sequence | | | |
| | GACAGCGAAGTAAAAAATGTTGATCTTGCATCACCAGAGGAACATCAGAAGCACCGAG | | |
| | AGATGGCTGTTGACTGCCCTGGAGATTTGGGCACCAGGATGATGCCAATACGTCGAAG | | |
| | TGCACAGTTGGAGCGTATTCGGCAACAACAGGAGGACATGAGGCGTAGGAGAGAGGAA | | |
| | GAAGGGAAAAAGCAAGAACTTGACCTTAATTCTTCCATGAGACTTAAGAAACTAGCCC | | |
| | AAATTCCTCCAAAGACCGGAATAGATAACCCTATGTTTGATACAGAGGAAGGAATTGT | | |
| | CTTAGAAAGTCCTCATTATGCTGTGAAAATATTAGAAATAGAAGACTTGTTTTCTTCA | | |
| | CTTAAACATATCCAACATACTTTGGTAGATTCTCAGAGCCAGGAGGATATTTCACTGC | | |
| | TTTTACAACTTGTTCAAAATAAGGATTTCCAGAATGCATTTAAGATACACAATGCCAT | | |
| | CACAGTACACATGAACAAGGCCAGTCCTCCATTTCCTCTTATCTCCAACGCACAAGAT | | |
| | CTTGCTCAAGAGGTACAAACTGTTTTGAAGCCAGTTCATCATAAGGAAGGACAAGAAC | | |
| | TAACTGCTTTGCTGAATACTCCACATATTCAGGCACTTTTACTGGCCCACGATAAGGT | | |
| | TGCTGAGCAGGAAATGCAGCTAGAGCCCATTACAGATGAGAGAGTTTATGAAAGTATT | | |
| | GGCCAGTATGGAGGAGAAACTGTAAAAATAGTTCGTATAGAAAAGGCTCGTGATATTC | | |
| | CGTTGGGTGCTACAGTTCGTAATGAAATGGACTCTGTCATCATTAGCCGGATAGTAAA | | |
| | AGGGGGTGCTGCAGAGAAAAGTGGTCTGTTGCATGAAGGAGATGAAGTTCTAGAGATT | | |
| | AATGGCATTGAAATTCGGGGGAAAGATGTCAATGAGGTTTTTGACTTGTTGTCTGATA | | |
| | TGCATGGTACTTTGACTTTTGTCCTGATTCCCAGTCAACAGATCAAGCCGCCTCCTGC | | |
| | CAAGGAAACAGTAATCCATGTAAAAGCTCATTTTGACTATGACCCCTCAGATGACCCT | | |
| | TATGTTCCATGTCGAGAGTTAGGTCTGTCTTTTCAAAAAGGTGATATACTTCATGTGA | | |
| | TCAGTCAAGAAGATCCAAACTGGTGGCAGGCCTACAGGGAAGGGGACGAAGATAATCA | | |
| | ACCTCTAGCCGGGCTTGTTCCAGGGAAAAGCTTTCAGCAGCAAAGGGAAGCCATGAAA | | |
| | CAAACCATAGAAGAAGATAAGGAGCCAGAAAAATCAGGTAAACTGTGGTGTGCAAAGA | | |
| | AGAATAAAAAGAAGAGGAAAAAGGTTTTATATAATGCCAATAAAAATGATGATTATGA | | |
| | CAACGAGGAGATCTTAACCTATGAGGAAATGTCACTTTATCATCAGCCAGCAAATAGG | | |
| | AAGAGACCTATCATCTTGATTGGTCCACAGAACTGTGGCCAGAATGAATTGCGTCAGA | | |
| | GGCTCATGAACAAAGAAAAGGACCGCTTTGCATCTGCAGTTCCTCGTACAACCCGGAG | | |
| | TAGGCGAGACCAAGAAGTAGCCGGTAGAGATTACCACTTTGTTTCGCGGCAAGCATTC | | |
| | GAGGCAGACATAGCAGCTGGAAAGTTCATTGAGCATGGTGAATTTGAGAAGAATTTGT | | |
| | ATGGAACTAGCATAGATTCTGTACGGCAAGTGATCAACTCTGGCAAAATATGTCTTTT | | |
| | AAGTCTTCGTACACAGTCATTGAAGACTCTCCGGAATTCAGATTTGAAACCATATATT | | |
| | ATCTTCATTGCACCCCCTTCACAAGAAAGACTTCGGGCATTATTGGCCAAAGAAGGCA | | |
| | AGAATCCAAAGCCTGAAGAGTTGAGAGAAATCATTGAGAAGACAAGAGAGATGGAGCA | | |
| | GAACAATGGCCACTACTTTGATACGGCAATTGTGAATTCCGATCTTGATAAAGCCTAT | | |
| | CAGGAATTGCTTAGGTTAATTAACAAACTTGATACTGAACCTCAGTGGGTACCATCCA | | |
| | CTTGGCTGAGGTGAAAGAAACATCCATTCT | | |
| | ORF Start: ATG at 17 | | ORF Stop: TGA at 2042 |
| | SEQ ID NO:22 | 675 aa | MW at 77311.8 kD |
| NOV9a, | MTTSHMNGHVTEESDSEVKNVDLASPEEHQKHREMAVDCPGDLGTRMMPIRRSAQLER | | |
| CG58590-01 Protein Sequence | | | |
| | IRQQQEDMRRRREEEGKKQELDLNSSMRLKKLAQIPPKTGIDNPMFDTEEGIVLESPH | | |
| | YAVKILEIEDLFSSLKHIQHTLVDSQSDEDISLLLQLVQNKDFQNAFKIHNAITVHMN | | |
| | KASPPFPLISNAQDLAQEVQTVLKPVHHKEGQELTALLNTPHIQALLLAHDKVAEQEM | | |
| | QLEPITDERVYESIGQYGGETVKIVRIEKARDIPLGATVRNEMDSVIISRIVKGGAAE | | |
| | KSGLLHEGDEVLEINGIEIRGKDVNEVFDLLSDMHGTLTFVLIPSQQIKPPPAKETVI | | |
| | HVKAHFDYDPSDDPYVPCRELGLSFQKGDILHVISQEDPNWWQAYREGDEDNQPLAGL | | |
| | VPGKSFQQQREAMKQTIEEDKEPEKSGKLWCAKKNKKKRKKVLYNANKNDDYDNEEIL | | |
| | TYEEMSLYHQPANRKRPIILIGPQNCGQNELRQRLMNKEKDRFASAVPRTTRSRRDQE | | |
| | VAGRDYHFVSRQAFEADIAAGKFIEHGEFEKNLYGTSIDSVRQVINSGKICLLSLRTQ | | |
| | SLKTLRNSDLKPYIIFIAPPSQERLRALLAKEGKNPKPEELREIIEKTREMEQNNGHY | | |
| | FDTAIVNSDLDKAYQELLRLINKLDTEPQWVPSTWLR | | |
| | SEQ ID NO:23 | 2030 bp | |
| NOV9b, | CCATGACAACATCCCATATGAATGGGCATGTTACAGAGGAATCAGACAGCGAAGTAAA | | |
| CG58590-02 DNA Sequence | | | |
| | AAATGTTGATCTTGCATCACCAGAGGAACATCAGAAGCACCGAGAGATGGCTGTTGAC | | |
| | TGCCCTGGAGATTTGGGCACCAGGATGATGCCAATACGTCGAAGTGCACAGTTGGAGC | | |
| | GTATTCGGCAACAACAGGAGGACATGAGGCGTAGGAGAGAGGAAGAAGGGAAAAAGCA | | |
| | AGAACTTGACCTTAATTCTTCCATGAGACTTAAGAAACTAGCCCAAATTCCTCCAAAG | | |
| | ACCGGAATAGATAACCCTATGTTTGATACAGAGGAAGGAATTGTCTTAGAAAGTCCTC | | |
| | ATTATGCTGTGAAAATATTAGAAATAGAAGACTTGTTTTCTTCACTTAAACATATCCA | | |
| | ACATACTTTGGTAGATTCTCAGAGCCAGGAGGATATTTCACTGCTTTTACAACTTGTT | | |
| | CAAAATAAGGATTTCCAGAATGCATTTAAGATACACAATGCCATCACAGTACATATGA | | |
| | ACAAGGCCAGTCCTCCATTTCCTCTTATCTCCAACGCACAAGATCTTGCTCAAGAGGT | | |
| | ACAAACTGTTTTGAAGCCAGTTCATCATAAGGAAGGACAAGAACTAACTGCTTTGCTG | | |
| | AATACTCCACATATTCAGGCACTTTTACTGGCCCACGATAAGGTTGCTGAGCAGGAAA | | |
| | TGCAGCTAGAGCCCATTACAGATGAGAGAGTTTATGAAAGTATTGGCCAGTATGGAGG | | |
| | AGAAACTGTAAAAATAGTTCGTATAGAAAAGGCTCGTGATATTCCGTTGGGTGCTACA | | |
| | GTTCGTAATGAAATGGACTCTGTCATCATTAGCCGGATAGTAAAAGGGGGTGCTGCAG | | |
| | AGAAAAGTGGTCTGTTGCATGAAGGAGATGAAGTTCTAGAGATTAATGGCATTGAAAT | | |
| | TCGGGGGAAAGATGTCAATGAGGTTTTTGACCTGTTGTCTGATATGCATGGTACTTTG | | |
| | ACTTTTGTCCTGATTCCCAGTCAACAGATCAAGCCGCCTCCTGCCAAGGAAACAGTAA | | |
| | TCCATGTAAAAGCTCATTTTGACTATGACCCCTCAGATGACCCTTATGTTCCATGTCG | | |
| | AGAGTTAGGTCTGTCTTTTCAAAAAGGTGATATACTTCATGTGATCAGTCAAGAAGAT | | |
| | CCAAACTGGTGGCAGGCCTACAGGGAAGGGGACGAAGATAATCAACCTCTAGCCGGGC | | |
| | TTGTTCCAGGGAAAAGCTTTCAGCAGCAAAGGGAAGCCATGAAACAAACCATAGAAGA | | |
| | AGATAAGGAGCCAGAAAAATCAGGAAAACTGTGGTGTGCAAAGAAGAATAAAAAGAAG | | |
| | AGGAAAAAGGTTTTATATAATGCCAATAAAAATGATGATTATGACAACGAGGAGATCT | | |
| | TAACCTATGAGGAAATGTCACTTTATCATCAGCCAGCAAATAGGAAGAGACCTATCAT | | |
| | CTTGATTGGTCCACAGAACTGTGGCCAGAATGAATTGCGTCAGAGGCTCATGAACAAA | | |
| | GAAAAGGACCGCTTTGCATCTGCAGTTCCTCATACAACCCGGAGTAGGCGAGACCAAG | | |
| | AAGTAGCCGGTAGAGATTACCACTTTGTTTCGCGGCAAGCATTCGAGGCAGACATAGC | | |
| | AGCTGGAAAGTTCATTGAGCATGGTGAATTTGAGAAGAATTTGTATGGAACTAGCATA | | |
| | GATTCTGTACGGCAAGTGATCAACTCTGGCAAAATATGTCTTTTAAGTCTTCGTACAC | | |
| | AGTCATTGAAGACTCTCCGGAATTCAGATTTGAAACCATATATTATCTTCATTGCACC | | |
| | CCCTTCACAAGAAAGACTTCGGGCATTATTGGCCAAAGAAGGCAAGAATCCAAAGCCT | | |
| | GAAGAGTTGAGAGAAATCATTGAGAAGACAAGAGAGATGGAGCAGAACAATGGCCACT | | |
| | ACTTTGATACGGCAATTGTGAATTCCGATCTTGATAAAGCCTATCAGGAATTGCTTAG | | |
| | GTTAATTAACAAACTTGATACTGAACCTCAGTGGGTACCATCCACTTGGCTGAGGTGA | | |
| | ORF Start: ATG at 3 | | ORF Stop: TGA at 2028 |
| | SEQ ID NO:24 | 675 aa | MW at 77292.8 kD |

| | |
|---|---|
| NOV9b, | MTTSEMNGEVTEESDSEVKNVDLASPEEHQKHREMAVDCPGDLGTRMMPIRRSAQLER |
| CG58590-02 Protein Sequence | |
| | IRQQQEDMRRRREEEGKKQELDLNSSMRLKKLAQIPPKTGIDNPMFDTEEGIVLESPH |
| | YAVKILEIEDLFSSLKHIQHTLVDSQSDEDISLLLQLVQNKDFQNAFKIHNAITVHMN |
| | KASPPFPLISNAQDLAQEVQTVLKPVHHKEGQELTALLNTPHIQALLLAHDKVAEQEM |
| | QLEPITDERVYESIGQYGGETVKIVRIEKARDIPLGATVRNEMDSVIISRIVKGGAAE |
| | KSGLLHEGDEVLEINGIEIRGKDVNEVFDLLSDMHGTLTFVLIPSQQIKPPPAKETVI |
| | HVKAHFDYDPSDDPYVPCRELGLSFQKGDILHVISQEDPNWWQAYREGDEDNQPLAGL |
| | VPGKSFQQQREAMKQTIEEDKEPEKSGKLWCAKKNKKKRKKVLYNANKNDDYDNEEIL |
| | TYEEMSLYHQPANRKRPIILIGPQNCGQNELRQRLMNKEKDRFASAVPHTTRSRRDQE |
| | VAGRDYHFVSRQAFEADIAAGKFIEHGEFEKNLYGTSIDSVRQVINSGKICLLSLRTQ |
| | SLKTLRNSDLKPYIIFIAPPSQERLRALLAKEGKNPKPEELREIIEKTREMEQNNGHY |
| | FDTAIVNSDLDKAYQELLRLINKLDTEPQWVPSTWLR |

Sequence comparison of the above protein sequences yields the following sequence relationships shown in Table 9B.

TABLE 9B
Comparison of NOV9a against NOV9b.

| | | Identities/ |
|---|---|---|
| Protein | NOV9a Residues/ | Similarities for |
| Sequence | Match Residues | the Matched Region |
| NOV9b | 1 . . . 675 | 636/675 (94%) |
| | 1 . . . 675 | 636/675 (94%) |

Further analysis of the NOV9a protein yielded the following properties shown in Table 9C.

TABLE 9C
Protein Sequence Properties NOV9a

| | |
|---|---|
| PSort | 0.7000 probability located in nucleus; 0.1000 probability |
| analysis: | located in mitochondrial matrix space; 0.1000 probability |
| | located in lysosome (lumen); 0.0000 probability located |
| | in endoplasmic reticulum (membrane) |
| SignalP | No Known Signal Sequence Predicted |
| analysis: | |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB94180 | Human protein sequence SEQ ID | 173 . . . 675 | 501/503 (99%) | 0.0 |
| | NO:14494 - Homo sapiens, 503 aa. | 1 . . . 503 | 501/503 (99%) | |
| | [EP1074617-A2, 7 FEB 2001] | | | |
| AAB41921 | Human ORFX ORF1685 | 406 . . . 675 | 261/270 (96%) | e−147 |
| | polypeptide sequence SEQ ID | 1 . . . 269 | 264/270 (97%) | |
| | NO:3370 - Homo sapiens, 269 aa. | | | |
| | [WO200058473-A2, 5 OCT 2000] | | | |
| AAU07123 | Human novel human protein, NHP | 143 . . . 674 | 224/564 (39%) | e−109 |
| | #23 - Homo sapiens, 576 aa. | 31 . . . 574 | 339/564 (59%) | |
| | [WO200161016-A2, 23 AUG 2001] | | | |
| AAU07119 | Human novel human protein, NHP | 143 . . . 654 | 213/544 (39%) | e−102 |
| | #19 - Homo sapiens, 560 aa. | 31 . . . 554 | 327/544 (59%) | |
| | [WO200161016-A2, 23 AUG 2001] | | | |
| AAU07115 | Human novel human protein, NHP | 143 . . . 606 | 196/481 (40%) | 5e−97 |
| | #15 - Homo sapiens, 520 aa. | 31 . . . 495 | 300/481 (61%) | |
| | [WO200161016-A2, 23 AUG 2001] | | | |

In a BLAST search of public sequence databases, the NOV9a protein was found to have homology to the proteins shown in the BLASTP data in Table 9E.

TABLE 9E
Public BLASTP Results for NOV9a

| | | | Identities/ | |
|---|---|---|---|---|
| | | NOV9a | Similarities | |
| Protein | | Residues/ | for the | |
| Accession | | Match | Matched | Expect |
| Number | Protein/Organism/Length | Residues | Portion | Value |
| Q9JLB2 | PALS1 - Mus musculus (Mouse), | 1 . . . 675 | 652/675 (96%) | 0.0 |
| | 675 aa. | 1 . . . 675 | 665/675 (97%) | |
| Q9H9Q0 | CDNA FLJ12615 FIS, CLONE | 173 . . . 675 | 501/503 (99%) | 0.0 |
| | NT2RM4001629, WEAKLY | 1 . . . 503 | 501/503 (99%) | |
| | SIMILAR TO MAGUK P55 | | | |
| | SUBFAMILY MEMBER 3 - Homo | | | |
| | sapiens (Human), 503 aa. | | | |
| AAL40935 | STARDUST PROTEIN MAGUK1 | 252 . . . 674 | 252/460 (54%) | e−140 |
| | ISOFORM - Drosophila | 829 . . . 1282 | 327/460 (70%) | |
| | melanogaster (Fruit fly), 1289 aa. | | | |
| Q9W3H6 | CG1617 PROTEIN - Drosophila | 252 . . . 674 | 252/500 (50%) | e−132 |
| | melanogaster (Fruit fly), 794 aa. | 294 . . . 787 | 327/500 (65%) | |
| Q9W7F1 | P55-RELATED MAGUK | 142 . . . 673 | 209/556 (37%) | e−105 |
| | PROTEIN DLG3 - Brachydanio | 30 . . . 573 | 335/556 (59%) | |
| | rerio (Zebrafish) (Zebra danio), 576 | | | |
| | aa. | | | |

PFam analysis predicts that the NOV9a protein contains the domains shown in the Table 9F.

TABLE 9F
Domain Analysis of NOV9a

| | | Identities/ | | |
|---|---|---|---|---|
| | | Similarities | | |
| Pfam Domain | NOV9a Match Region | for the Matched Region | | Expect Value |
| L27: domain 1 of 1 | 186 . . . 238 | 19/56 | (34%) | 0.049 |
| | | 39/56 | (70%) | |
| PDZ: domain 1 of 1 | 256 . . . 335 | 21/83 | (25%) | 9.7e−12 |
| | | 58/83 | (70%) | |
| SH3: domain 1 of 1 | 348 . . . 415 | 19/68 | (28%) | 0.026 |
| | | 46/68 | (68%) | |
| Guanylate_kin: domain 1 | 515 . . . 624 | 54/113 | (48%) | 6.2e−38 |
| of 1 | | 87/113 | (77%) | |
| Peptidase_S15: domain 1 | 642 . . . 658 | 6/17 | (35%) | 8.2 |
| of 1 | | 13/17 | (76%) | |
| Caulimo_mov: domain 1 | 420 . . . 673 | 59/335 | (18%) | 6.1 |
| of 1 | | 156/335 | (47%) | |

### Example 10

The NOV10 clone was analyzed, and the nucleotide and predicted polypeptide sequences are shown in Table 10A.

TABLE 10A
NOV10 Sequence Analysis

| | SEQ ID NO:25 | 576 bp | |
|---|---|---|---|
| NOV10a, | ACCTTACTAGAAAAATGAAACCTGATGAAACTCCTATGTTTGACCCAAGTCTACTCAA | | |
| CG58572-01 DNA Sequence | | | |
| | AGAAGTGGACTGGAGTCAGAATACAGCTACATTTTCTCCAGCCATTTCCCCAACACAT | | |
| | CCTGGAGAAGGCTTGGTTTTGAGGCCTCTTTGTACTGCTGACTTAAATAGAGGTTTTT | | |
| | TTAAGGTATTGGGTCAGCTAACAGAGACTGGAGTTGTCAGCCCTGAACAATTTATGGA | | |
| | ATCTTTTGAGCATATGAAGAAATCTGGGGATTATTATGTTACAGTTGTAGAAGATGTG | | |
| | ACTCTAGGACAGATTGTTGCTACGGCAACTCTGATTATAGAACATAAATTCATCCATT | | |
| | CCTGTGCTAAGAGAGGAAGAGTAGAAGATGTTGTTGTTAGTGATGAATGCAGAGGAAA | | |
| | GCAGCTTGGCAAATTGTTATTATCAACCCTTACTTTGCTAAGCAAGAAACTGAACTGT | | |
| | TACAAGATTACCCTTGAATGTCTACCACAAAATGTTGGTTTCTATAAAAAGTTTGGAT | | |
| | ATACTGTATCTGAAGAAAACTACATGTGTCGGAGGTTTCTAAAGTAAAAATCTT | | |
| | ORF Start: ATG at 15 | | ORF Stop: TAA at 567 |
| | SEQ ID NO:26 | 184 aa | MW at 2079.9 kD |
| NOV10a, | MKPDETPMFDPSLLKEVDWSQNTATFSPAISPTHPGEGLVLRPLCTADLNRGFFKVLG | | |
| CG58572-01 Protein Sequence | | | |
| | QLTETGVVSPEQFMESFEHMKKSGDYYVTVVEDVTLGQIVATATLIEHKFIHSCAKR | | |
| | GRVEDVVVSDECRGKQLGKLLLSTLTLLSKKLNCYKITLECLPQNVGFYKKFGYTVSE | | |
| | ENYMCRRFLK | | |
| | SEQ ID NO:27 | 560 bp | |
| NOV10b, | ATGAAACCTGATGAAACTCCTATGTTTGACCCAAGTCTACTCAAAGAAGTGGACTGGA | | |
| CG58572-02 DNA Sequence | | | |
| | GTCAGAATACAGCTACATTTTCTCCAGCCATTTCCCCAACACATCCTGGAGAAGGCTT | | |
| | GGTTTTGGGGCCTCTTTGTACTGCTGACTTAAATAGAGGTTTTTTTAAGGTATTGGGT | | |
| | CAGCTAACAGAGACTGGAGTTGTCAGCCCTGAACAATTTATGAAATCTTTTGAGCATA | | |
| | TGAAGAAATCTGGGGATTATTATGTTACAGTTGTAGAAGATGTGACTCTAGGACAGAT | | |
| | TGTTGCTACGGCAACTCTGATTATAGAACATAAATTCATCCATTCCTGTGCTAAGAGA | | |
| | GGAAGAGTAGAAGATGTTGTTGTTAGTGATGAATGCAGAGGAAAGCAGCTTGGCAAAT | | |
| | TGTTATTATCAACCCTTACTTTGCTAAGCAAGAAACTGAACTGTTACAAGATTACCCT | | |
| | TGAATGTCTACCACAAAATGTTGGTTTCTATAAAAAGTTTGGATATACTGTATCTGAA | | |
| | GAAAACTACATGTGTCGGAGGTTTCTAAAGTAAAAATC | | |
| | ORF Start: ATG at 1 | | ORF Stop: TAA at 553 |
| | SEQ ID NO:28 | 184 aa | MW at 20649.8 kD |

| | |
|---|---|
| NOV10b, | MKPDETPMFDPSLLKEVDWSQNTATFSPAISPTHPGEGLVLGPLCTADLNRGFFKVLG |
| CG5872-02 Protein Sequence | |
| | QLTETGVVSPEQFMKSFEHMKKSGDYYVTVVEDVTLGQIVATATLIIEHKFIHSCAKR |
| | GRVEDVVVSDECRGKQLGKLLLSTLTLLSKKLNCYKITLECLPQNVGFYKKFGYTVSE |
| | ENYMCRRFLK |

Sequence comparison of the above protein sequences yields the following sequence relationships shown in Table 10B.

TABLE 10B
Comparison of NOV10a against NOV10b.

| | | Identities/ |
|---|---|---|
| Protein | NOV10a Residues/ | Similarities for |
| Sequence | Match Residues | the Matched Region |
| NOV10b | 1 . . . 184 | 163/184 (88%) |
| | 1 . . . 184 | 164/184 (88%) |

Further analysis of the NOV10a protein yielded the following properties shown in Table 10C.

TABLE 10C
Protein Sequence Properties NOV10a

| | |
|---|---|
| PSort | 0.4500 probability located in cytoplasm; 0.1206 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG67123 | Amino acid sequence of human | 1 . . . 184 | 183/184 (99%) | e−105 |
| | 50287 transferase - Homo sapiens, | 1 . . . 184 | 184/184 (99%) | |
| | 184 aa. [WO200164904-A2, | | | |
| | 7 SEP 2001] | | | |
| AAB73505 | Human transferase HTFS-12, SEQ | 1 . . . 184 | 183/184 (99%) | e−105 |
| | ID NO: 12 - Homo sapiens, 184 aa. | 1 . . . 184 | 184/184 (99%) | |
| | [WO200132888-A2, 10 MAY | | | |
| | 2001] | | | |
| AAB63700 | Human gastric cancer associated | 1 . . . 184 | 183/184 (99%) | e−105 |
| | antigen protein sequence SEQ ID | 17 . . . 200 | 184/184 (99%) | |
| | NO:1062 - Homo sapiens, 200 aa. | | | |
| | [WO200073801-A2, 7 DEC | | | |
| | 2000] | | | |
| AAU07779 | Human novel transferase protein, | 1 . . . 184 | 182/184 (98%) | e−104 |
| | NHP #22 - Homo sapiens, 184 aa. | 1 . . . 184 | 183/184 (98%) | |
| | [WO200164903-A2, 7 SEP 2001] | | | |
| AAM79992 | Human protein SEQ ID NO 3638 - | 1 . . . 184 | 181/184 (98%) | e−104 |
| | Homo sapiens, 206 aa. | 23 . . . 206 | 183/184 (99%) | |
| | [WO200157190-A2, 9 AUG | | | |
| | 2001] | | | |

In a BLAST search of public sequence databases, the NOV10a protein was found to have homology to the proteins shown in the BLASTP data in Table 10E.

TABLE 10E
Public BLASTP Results for NOV10a

| | | | Identities/ | | |
|---|---|---|---|---|---|
| | | NOV10a | Similarities | | |
| Protein | | Residues/ | for the | | |
| Accession | | Match | Matched | | Expect |
| Number | Protein/Organism/Length | Residues | Portion | | Value |
| Q96EK6 | SIMILAR TO GLUCOSAMINE- | 1 . . . 184 | 183/184 | (99%) | e−104 |
| | PHOSPHATE N- | 1 . . . 184 | 184/184 | (99%) | |
| | ACETYLTRANSFERASE - Homo | | | | |
| | sapiens (Human), 184 aa. | | | | |
| Q9JK38 | EMEG32 PROTEIN | 1 . . . 184 | 180/184 | (97%) | e−102 |
| | (GLUCOSAMINE-PHOSPHATE N- | 1 . . . 184 | 182/184 | (98%) | |
| | ACETYLTRANSFERASE) - Mus | | | | |
| | musculus (Mouse), 184 aa. | | | | |
| Q9VAI0 | Probable glucosamine-phosphate N- | 4 . . . 176 | 84/174 | (48%) | 2e−43 |
| | acetyltransferase (EC 2.3.1.4) | 6 . . . 179 | 123/174 | (70%) | |
| | (Phosphoglucosamine transacetylase) | | | | |
| | (Phosphoglucosamine acetylase) - | | | | |
| | Drosophila melanogaster (Fruit fly), | | | | |
| | 219 aa. | | | | |
| Q17427 | Probable glucosamine-phosphate N- | 32 . . . 182 | 65/152 | (42%) | 1e−28 |
| | acetyltransferase (EC 2.3.1.4) | 15 . . . 165 | 98/152 | (63%) | |
| | (Phosphoglucosamine transacetylase) | | | | |
| | (Phosphoglucosamine acetylase) - | | | | |
| | Caenorhabditis elegans, 165 aa. | | | | |
| O45811 | T23G11.2PROTEIN - | 42 . . . 184 | 63/143 | (44%) | 3e−26 |
| | Caenorhabditis elegans, 347 aa. | 201 . . . 340 | 88/143 | (61%) | |

PFam analysis predicts that the NOV10a protein contains the domains shown in the Table 10F.

TABLE 10F
Domain Analysis of NOV10a

| | | Identities/ | |
|---|---|---|---|
| | | Similarities | |
| Pfam Domain | NOV10a Match Region | for the Matched Region | Expect Value |
| Acetyltransf: domain 1 | 89 . . . 171 | 22/87 (25%) | 6.5e−13 |
| of 1 | | 62/87 (71%) | |

### Example 11

The NOV11 clone was analyzed, and the nucleotide and predicted polypeptide sequences are shown in Table 11A.

TABLE 11A
NOV11 Sequence Analysis

| | SEQ ID NO:29 | 7098 bp | |
|---|---|---|---|
| NOV11a, | CCCGCGGGCCAGCACCATGGAGGACGTGAAGCTGGAGTTCCCTTCCCTTCCACAGTGC | | |
| CG58564-01 DNA Sequence | | | |
| | AAGGAAGACGCCGAGGAGTGGACCTACCCTATGAGACGAGAGATGCAGGAAATTTTAC | | |
| | CTGGATTGTTCTTAGGCCCATATTCATCTGCTATGAAAAGCAAGCTACCTGTACTACA | | |
| | GAAACATGGAATAACCCATATAATATGCATACGACAAAATATTGAAGCAAACTTTATT | | |
| | AAACCAAACTTTCAGCAGTTATTTAGGTATTTAGTCCTGGATATTGCAGATAATCCAG | | |
| | TTGAAAATATAATACGTTTTTTCCCTATGACTAAGGAATTTATTGATGGGAGCTTACA | | |
| | AATGGGAGGTAAAGTTCTTGTGCATGGAAATGCAGGGATCTCCAGAAGTGCAGCCTTT | | |
| | GTTATTGCATACATTATGGAAACATTTGGAATGAAGTACAGGGATGCTTTTGCTTATG | | |
| | TTCAAGAAAGAAGATTTTGTATTAATCCTAATGCTGGATTTGTCCATCAACTTCAGGA | | |
| | ATATGAAGCCATCTACCTAGCAAAATTAACAATACAGATGATGTCACCACTCCAGATA | | |
| | GAAAGGTCATTATCTGTTCATTCTGGTACCACAGGTAGTTTGAAGAGAACACATGAAG | | |
| | AAGAGGATGATTTTGGAACCATGCAAGTGGCGACTGCACAGAATGGCTGACTTGAAGA | | |
| | GCAACATCATAGA | | |
| | ORF Start: ATG at 17 | | ORF Stop: TGA at 686 |
| | SEQ ID NO:30 | 223 aa | MW at 25492.2 kD |
| NOV11a, | MEDVKLEFPSLPQCKEDAEEWTYPMRREMQEILPGLEFLGPYSSAMKSKLPVLQKHGIT | | |
| CG58564-01 Protein Sequence | | | |
| | HIICIRQNIEANFIKPNFQQLFRYLVLDIADNPVENIIRFFPMTKEFIDGSLQMGGKV | | |
| | LVHGNAGISRSAAFVIAYIMETFGMKYRDAFAYVQERRFCINPNAGFVHQLQEYEAIY | | |
| | LAKLTIQMMSPLQIERSLSVHSGTTGSLKRTHEEEDDPGTMQVATAQNG | | |
| | SEQ ID NO:31 | 724 bp | |
| NOV 11b, | ACTCTCCCACCCCACCCACCAGAATGGCGGGCCAGCACCATGGAGGACGTGAAGCTGG | | |
| CG58564-02 DNA Sequence | | | |
| | AGTTCCCTTCCCTTCCACAGTGCAAGGAAGACGCCGAGGAGTGGACCTACCCTATGAG | | |
| | ACGAGAGATGCAGGAAATTTTATCTGGATTGTTCTTAGGCCCATATTCATCTGCTATG | | |
| | AAAAGCAAGCTACCTGTACTACAGAAACATGGAATAACCCATATAATATGCATACGAC | | |
| | AAAATATTGAAGCAAACTTTATTAAACCAAACTTTCAGCAGTTATTTAGATATTTAGT | | |
| | CCTGGATATTGCAGATAATCCAGTTGAAAATATAATACGTTTTTTCCCTATGACTAAG | | |
| | GAATTTATTGATGGGAGCTTACAAATGGGAGGAAAAGTTCTTGTGCATGGAAATGCAG | | |
| | GGATCTCCAGAAGTGCAGCCTTTGTTATTGCATACATTATGGAAACATTTGGAATGAA | | |
| | GTACAGAGATGCTTTTGCTTATGTTCAAGAAAGAAGATTTTGTATTAATCCTAATGCT | | |
| | GGATTTGTCCATCAACTTCAGGAATATGAAGCCATCTACCTAGCAAAATTAACAATAC | | |
| | AGATGATGTCACCACTCCAGATAGAAAGGTCATTATCTGTTCATTCTGGTACCACAGG | | |
| | CAGTTTGAAGAGAACACATGAAGAAGAGGATGATTTTGGAACCATGCAAGTGGCGACT | | |
| | GCACAGAATGGTGACTTGAAGAGCAAC | | |
| | ORF Start: ATG at 40 | | ORF Stop: TGA at 709 |
| | SEQ ID NO:32 | 223 aa | MW at 25482.1 kD |
| NOV11b, | MEDVKLEFPSLPQCKEDAEEWTYPMRREMQEILSGLFLGPYSSAMKSKLPVLQKHGIT | | |
| CG58564-02 Protein Sequence | | | |
| | HIICIRQNIEANFIKPNFQQLFRYLVLDIADNPVENIIRFFPMTKEFIDGSLQMGGKV | | |
| | LVHGNAGISRSAAFVIAYIMETFGMKYRDAFAYVQERRFCINPNAGFVHQLQEYEAIY | | |
| | LAKLTIQMMSPLQIERSLSVHSGTTGSLKRTHEEEDDFGTMQVATAQNG | | |
| | SEQ ID NO:33 | 545 bp | |
| NOV11c, | ACTCTCCCACCCCACCCACCAGCCCGCGGGCCAGCACCATGGAGGACGTGAAGCTGGA | | |
| CG58564-03 DNA Sequence | | | |
| | GTTCCCTTCCCTTCCACAGTGCAAGGAAGACGCCGAGGAGTGGACCTACCCTATGAGA | | |
| | CGAGAGATGCAGGAAATTTTACCTGGATTGTTCTTAGGCCCATATTCATCTGCTATGA | | |
| | AAAGCAAGCTACCTGTACTACAGAAACATTTGGAATGAAGTACAGAGATGCTTTTGCT | | |
| | TATGTTCAAGAAAGAAGATTTTGTATTAATCCTAATGCTGGATTTGTCCATCAACTTC | | |
| | AGGAATATGAAGCCATCTACCTAGCAAAATTAACAATACAGATGATGTCACCACTCCA | | |
| | GATAGAAAGGTCATTATCTGTTCATTCTGGTACCACAGGCAGTTTGAAGAGAACACAT | | |
| | GAGGAAGAGGATGATTTTGGAACCATGCAAGTGGCGACTGCACAGAATGGCTGACTTG | | |
| | AAGAGCAACATCATAGAGTGTGAATTTCTATTTGGGAAGGAGAAAATACAAGAGAAAA | | |
| | TTATAATGTAAAATGGTAAAAAA | | |
| | ORF Start: ATG at 39 | | ORF Stop: TGA at 210 |
| | SEQ ID NO:34 | 57 aa | MW at 6695.7 kD |
| NOV11c, | MEDVKLEFPSLPQCKEDAEEWTYPMRREMQEILPGLFLGPYSSAMKSKLPVLQKHLE | | |
| CG58564-03 DNA Sequence | | | |
| | SEQ ID NO:35 | 663 bp | |
| NOV11d, | ACTCTCCCACCCCACCCACCAGCCCGCGGGCCAGCACCATGGAGGACGTGAAGCTGGA | | |
| CG58564-04 DNA Sequence | | | |
| | GTTCCCTTCCCTTCCACAGTGCAAGGAAGACGCCGAGGAGTGGACCTACCCTATGAGA | | |
| | CGAGAGATGCAGGAAATTTTACCTGGATTGTTCTTAGGCCCATATTCATCTGCTATGA | | |
| | AAAGCAAGCTACCTGTACTACAGAAACATGGAATAACCCATATAATATGCATACGACA | | |
| | AAATATTGAAGCAAACTTTATTAAACCAAACTTTCAGCAGTTATTTAGACTAAGGAAT | | |
| | TTATTGATGGGAGCTTACAAATGGGAGGAAAAGTTCTTGTGCATGGAAATGCAGGGAT | | |
| | CTCCAGAAGTGCAGCCTTTGTTATTGCATACATTATGGAAACATTTGGAATGAAGTAC | | |
| | AGAGATGCTTTTGCTTATGTTCAAGAAAGAAGATTTTGTATTAATCCTAATGCTGGAT | | |
| | TTGTCCATCAACTTCAGGAATATGAAGCCATCTACCTAGCAAAATTAACAATACAGAT | | |
| | GATGTCACCACTCCAGATAGAAAGGTCATTATCTGTTCATTCTGGTACCACAGGCAGT | | |
| | TTGAAGAGAACACATGAAGAAGAGGATGATTTTGGAACCATGCAAGTGGCGACTGCAC | | |
| | AGAATGGCTGACTTGAAGAGCAACT | | |
| | ORF Start: ATG at 39 | | ORF Stop: TGA at 399 |
| | SEQ ID NO:36 | 120 aa | MW at 14245.6 kD |

| | |
|---|---|
| NOV11d, | MEDVKLEFPSLPQCKEDAEEWTYPMRREMQEILPGLFLGPYSSAMKSKLPVLQKHGIT |
| CG58564-04 Protein Sequence | |
| | HIICIRQNIEANFIKPNFQQLEFRLRNLLMGAYKWEEKFLCMEMQGSPEVQPLLLHTLW |
| | KHLE |

Sequence comparison of the above protein sequences yields the following sequence relationships shown in Table 11B.

TABLE 11B
Comparison of NOV11a against NOV11b through NOV11d.

| | | Identities/ | |
|---|---|---|---|
| | NOV11a Residues/ | Similarities for | |
| Protein Sequence | Match Residues | the Matched Region | |
| NOV11b | 1 . . . 223 | 222/223 | (99%) |
| | 1 . . . 223 | 222/223 | (99%) |
| NOV11c | 1 . . . 55 | 55/55 | (100%) |
| | 1 . . . 55 | 55/55 | (100%) |
| NOV11d | 1 . . . 81 | 81/81 | (100%) |
| | 1 . . . 81 | 81/81 | (100%) |

Further analysis of the NOV11a protein yielded the following properties shown in Table 11C.

TABLE 11C
Protein Sequence Properties NOV11a

| | |
|---|---|
| PSort | 0.4698 probability located in microbody (peroxisome); 0.4500 |
| analysis: | probability located in cytoplasm; 0.1958 probability located in |
| | lysosome (lumen); 0.1000 probability located in mitochondrial |
| | matrix space |
| SignalP | No Known Signal Sequence Predicted |
| analysis: | |

A search of the NOV11a protein against the Geneseq database, a proprietary database that contains sequences published in patents and patent publication, yielded several homologous proteins shown in Table 11D.

TABLE 11D
Geneseq Results for NOV11a

| | | | Identities/ | |
|---|---|---|---|---|
| | | NOV11a | Similarities | |
| | Protein/ | Residues/ | for the | |
| Geneseq | Organism/Length | Match | Matched | Expect |
| Identifier | [Patent #, Date] | Residues | Region | Value |
| AAU09017 | Human dual | 1 . . . 223 | 223/223 | e−128 |
| | specificity | | (100%) | |
| | phosphatase | 1 . . . 223 | 223/223 | |
| | 38692—Homo | | (100%) | |
| | sapiens, 223 aa. | | | |
| | [WO200173059- | | | |
| | A2, Oct. 4, 2001] | | | |
| AAE08552 | Human | 1 . . . 223 | 223/223 | e−128 |
| | phosphatase | | (100%) | |
| | protein—Homo | 1 . . . 223 | 223/223 | |
| | sapiens, 223 aa. | | (100%) | |
| | [WO200160992- | | | |
| | A2, Aug. 23, | | | |
| | 2001] | | | |
| AAM41520 | Human poly- | 1 . . . 223 | 223/223 | e−128 |
| | peptide SEQ ID | | (100%) | |
| | NO 6451—Homo | 14 . . . 236 | 223/223 | |
| | sapiens, 236 aa. | | (100%) | |
| | [WO200153312- | | | |
| | A1, Jul. 26, 2001] | | | |
| AAM39734 | Human poly- | 1 . . . 223 | 223/223 | e−128 |
| | peptide SEQ ID | | (100%) | |
| | NO 2879—Homo | 1 . . . 223 | 223/223 | |
| | sapiens, 223 aa. | | (100%) | |
| | [WO200153312- | | | |
| | A1, Jul. 26, 2001] | | | |
| AAU23521 | Novel human | 25 . . . 171 | 55/147 | 1e−18 |
| | enzyme poly- | | (37%) | |
| | peptide #607— | 7 . . . 145 | 80/147 | |
| | Homo sapiens, | | (54%) | |
| | 190 aa. | | | |
| | [WO200155301- | | | |
| | A2, Aug. 2, | | | |
| | 2001] | | | |

In a BLAST search of public sequence databases, the NOV11a protein was found to have homology to the proteins shown in the BLASTP data in Table 11E.

TABLE 11E
Public BLASTP Results for NOV11a

| | | | Identities/ | |
|---|---|---|---|---|
| | | NOV11a | Similarities | |
| Protein | | Residues/ | for the | |
| Accession | Protein/ | Match | Matched | Expect |
| Number | Organism/Length | Residues | Portion | Value |
| CAD10219 | SEQUENCE 4 | 1 . . . 223 | 223/223 | e−127 |
| | FROM PATENT | | (100%) | |
| | WO0173059— | 1 . . . 223 | 223/223 | |
| | Homo sapiens | | (100%) | |
| | (Human), 223 aa. | | | |
| Q9DCF8 | 0610039A20RIK | 1 . . . 223 | 215/223 | e−124 |
| | PROTEIN—Mus | | (96%) | |
| | musculus | 1 . . . 223 | 221/223 | |
| | (Mouse), 223 aa. | | (98%) | |
| Q60970 | PROTEIN | 1 . . . 223 | 214/223 | e−124 |
| | TYROSINE | | (95%) | |
| | PHOSPHATASE- | 1 . . . 223 | 221/223 | |
| | LIKE—Mus | | (98%) | |
| | musculus | | | |
| | (Mouse), 223 aa. | | | |
| Q60969 | PROTEIN | 1 . . . 168 | 163/168 | 2e−93 |
| | TYROSINE | | (97%) | |
| | PHOSPHATASE- | 1 . . . 168 | 167/168 | |
| | LIKE—Mus | | (99%) | |
| | musculus | | | |
| | (Mouse), 205 aa. | | | |
| Q99850 | TYROSINE | 116 . . . 181 | 66/66 | 3e−31 |
| | PHOSPHATASE- | | (100%) | |
| | LIKE PROTEIN | 1 . . . 66 | 66/66 | |
| | HOMOLOG | | (100%) | |
| | HSTYXB— | | | |
| | Homo sapiens | | | |
| | (Human), 66 aa | | | |
| | (fragment). | | | |

PFam analysis predicts that the NOV11a protein contains the domains shown in the Table 11F.

TABLE 11F
Domain Analysis of NOV11a

| | | Identities/ | |
|---|---|---|---|
| | NOV11a | Similarities for | Expect |
| Pfam Domain | Match Region | the Matched Region | Value |
| DSPc: domain | 28 . . . 173 | 64/172 (37%) | 2.2e−63 |
| 1 of 1 | | 127/172 (74%) | |
| Y_phosphatase: | 35 . . . 179 | 35/279 (13%) | 1.7 |
| domain 1 of 1 | | 93/279 (33%) | |

### Example 12

The NOV12 clone was analyzed, and the nucleotide and predicted polypeptide sequences are shown in Table 12A.

TABLE 12A
NOV12 Sequence Analysis

| | SEQ ID NO:37 | 3696 bp | |
|---|---|---|---|
| NOV12a, | GTGTAAAAATACTGTCCATTTAATGTTTTCTGGGACTTTAGGTAAGAATATGAAAACT | | |
| CG57819-01 DNA Sequence | | | |
| | CAACCACCCTTGAGCAGGATGAACCGGGAGGAATTGGAGGACAGTTTCTTTCGACTTC | | |
| | GCGAAGATCACATGTTGGTGAAGGAGCTTTCTTGGAAGCAACAGGATGAGATCAAAAG | | |
| | GCTGAGGACCACCTTGCTGCGGTTGACCGCTGCTGGCCGGGACCTGCGGGTCGCGGAG | | |
| | GAGGCGGCGCCGCTCTCGGAGACCGCAAGGCGCGGGCAGAAGGCGGGATGGCGGCAGC | | |
| | GCCTCTCCATGCACCAGCGCCCCCAGATGCACCGACTGCAAGGGCATTTCCACTGCGT | | |
| | CGGCCCTGCCAGCCCCCGCCGCGCCCAGCCTCGCGTCCAAGTGGGACACAGACAGCTC | | |
| | CACACAGCCGGTGCACCGGTGCCGGAGAAACCCAAGAGGGGTAGGGACAGGCTGAGCT | | |
| | ACACAGCCCCTCCATCGTTTAAGGAGCATGCGACAAATGAAAACAGAGGTGAAGTAGC | | |
| | CAGTAAACCCAGTGAACTGGCCCACATCATGGCCAGCAATACCATGCAAGTGGAAGAG | | |
| | CCACCCAAGTCTCCTGAGAAAATGTGGCCTAAAGATGAAAATTTTGAACAGAGAAGCT | | |
| | CATTGGAGTGTGCTCAGAAGGCTGCAGAGCTTCGGGCTTCCATTAAAGAGAAGGTAGA | | |
| | GCTGATTCGACTTAAGAAGCTCTTACATGAAAGAAATGCTTCATTGGTTATGACAAAA | | |
| | GCACAATTAACAGAAGTTCAAGAGGTGAGTTGCCATCTTTTGACCCAGAATCAGGGAA | | |
| | TCCTGAGTGCAGCCCATGAGGCCCTCCTCAAGCAAGTGAATGAGCTCAGGGCAGAGCT | | |
| | GAAGGAAGAAAGCAAGAAGGCTGTGAGCTTGAAGAGCCAACTGGAAGATGTGTCTATC | | |
| | TTGCAGATGACTCTGAAGGAGTTTCAGGAGAGAGTTGAAGATTTGGAAAAAGAACGAA | | |
| | AATTGCTGAATGACAATTATGACAAACTCTTAGAAAGCAGTGACAGCTCCAGTCAGCC | | |
| | CCACTGGAGCAACGAGCTCATAGCGGAACAGCTACAGCAGCAAGTCTCTCAGCTGCAG | | |
| | GATCAGCTGGATGCTGAGCTGGAGGACAAGAGAAAAGTTTTACTTGAGCTGTCCAGGG | | |
| | AGAAAGCCCAAAATGAGGATCTGAAGCTTGAAGTCACCAACATACTTCAGAAGCATAA | | |
| | ACAGGAAGTAGAGCTCCTCCAAAATGCAGCCACAATTTCCCAACCTCCTGACAGGCAA | | |
| | TCTGAACCAGCCACTCACCCAGCTGTATTGCAAGAGAACACTCAGATCCAGCCAAGTG | | |
| | AACCCAAAAACCAAGAAGAAAAGAAACTGTCCCAGGTGCTAAATGAGTTGCAAGTATC | | |
| | ACACGCAGAGACCACATTGGAACTAGAAAAGACCAGGGACATGCTTATTCTGCAGCGC | | |
| | AAAATCAACGTGTGTTATCAGGAGGAACTGGAGGCAATGATGACAAAAGCTGACAATG | | |
| | ATAATAGAGATCACAAAGAAAAGCTGGAGAGGTTGACTCGACTACTAGACCTCAAGAA | | |
| | TAACCGTATCAAGCAGCTGGAAGAACAGCTCAAAGATGTTGCTTATGGCACCCGACCG | | |
| | TTGTCGTTATGTTTGGAAACACTGCCAGCCCATGGAGATGAGGATAAAGTGGATATTT | | |
| | CTCTGCTGCATCAGGGTGAGAATCTTTTTGAACTGCACATCCACCAGGCCTTCCTGAC | | |
| | ATCTGCCGCCCTAGCTCAGGCTGGAGATACCCAACCTACCACTTTCTGCACCTATTCC | | |
| | TTCTATGACTTTGAAACCCACTGTACCCCATTATCTGTGGGGCCACAGCCCCTCTATG | | |
| | ACTTCACCTCCCAGTATGTGATGGAGACAGATTCGCTTTTCTTACACTACCTTCAAGA | | |
| | GGCTTCAGCCCGGCTTGACATACACCAGGCCATGGCCAGTGAACACAGCACTCTTGCT | | |
| | GCAGGATGGATTTGCTTTGACAGGGTGCTAGAGACTGTGGAGAAAGTCCATGGCTTGG | | |
| | CCACACTGATTGGTGCTGGTGGAGAAGAGTTCGGGGTTCTAGAGTACTGGATGAGGCT | | |
| | GCGTTTCCCCATAAAACCCAGCCTACAGGCGTGCAATAAACGAAAGAAAGCCCAGGTC | | |
| | TACCTGTCAACCGATGTGCTTGGAGGCCGGAAGGCCCAGGAAGAGGAGGTGAGATCGG | | |
| | AGTCTTGGGAACCTCAGAACGAGCTGTGGATTGAAATCACCAAGTGCTGTGGCCTCCG | | |
| | GAGTCGATGGCTGGGAACTCAACCCAGTCCATATGCTGTGTACCGCTTCTTCACCTTT | | |
| | TCTGACCATGACACTGCCATCATTCCAGCCAGTAACAACCCCTACTTTAGAGACCAGG | | |
| | CTCGATTCCCAGTGCTTGTGACCTCTGACCTGGACCATTATCTGAGACGGGAGGCCTT | | |
| | GTCTATACATGTTTTTGATGATGAAGACTTAGAGCCTGGCTCGTATCTTGGCCGAGCC | | |
| | CGAGTGCCTTTACTGCCTCTTGCAAAAAATGAATCTATCAAAGGTGATTTTAACCTCA | | |
| | CTGACCCTGCAGAGAAACCCAACGGATCTATTCAAGTGCAACTGGATTGGAAGTTTCC | | |
| | CTACATACCCCCTGAGAGCTTCCTGAAACCAGAAGCTCAGACTAAGGGGAAGGATACC | | |
| | AAGGACAGTTCAAAGATCTCATCTGAAGAGGAAAAGGCTTCATTTCCTTCCCAGGATC | | |
| | AGATGGCATCTCCTGAGGTTCCCATTGAAGCTGGCCAGTATCGATCTAAGAGAAAACC | | |
| | TCCTCATGGGGGAGAAAGAAAGGAGAAGGAGCACCAGGTTGTGAGCTACTCAAGAAGA | | |
| | AAACATGGCAAAAGAATAGGTGTTCAAGGAAAGAATAGAATGGAGTATCTTAGCCTTA | | |
| | ACATCTTAAATGGAAATACACTGAAGCAGGTGAATTACACTGAGTGGAAGTTCTCAGA | | |
| | GACTAACAGCTTCATAGGTGATGGCTTTAAAAATCAGCACGAGGAAGAGGAAATGACA | | |
| | TTATCCCATTCAGCACTGAAACAGAAGGAACCTCTACATCCTGTAAATGACAAAGAAT | | |
| | CCTCTGAACAAGGTTCTGAAGTCAGTGAAGCACAAACTACCGACAGTGATGATGTCAT | | |
| | AGTGCCACCCATGTCTCAGAAATATCCTAAGGCAGATTCAGAGAAGATGTGCATTGAA | | |
| | ATTGTCTCCCTGGCCTTCTACCCAGAGGCAGAAGTGATGTCTGATGAGAACATAAAAC | | |
| | AGGTGTATGTGGAGTACAAATTCTACGACCTACCCTTGTCGGAGACAGAGACTCCAGT | | |
| | GTCCCTAAGGAAGCCTAGGGCAGGAGAAGAAATCCACTTTCACTTTAGCAAGGTAATA | | |
| | GACCTGGACCCACAGGAGCAGCAAGGCCGAAGGCGGTTTCTGTTCGACATGCTGAATG | | |
| | GACAAGATCCTGATCAAGGACAGTTAAAGTTTACAGTGGTAAGTGATCCTCTGGATGA | | |
| | AGAAAAGAAAGAATGTGAAGAAGTGGGATATGCATATCTTCAACTGTGGCAGATCCTG | | |
| | GAGTCAGGAAGAGATATTCTAGAGCAAGAGCTAGACGTTGTTAGCCCTGAAGATCTGG | | |
| | CTACCCCAATAGGAAGGCTGAAGGTTTCCCTTCAAGCAGCTGCTGTCCTCCATGCTAT | | |
| | TTACAAGGAGATGACTGAAGATTTGTTTTCATGAAGGAACAA | | |
| | ORF Start: ATG at 23 | | ORF Stop: TGA at 3686 |
| | SEQ ID NO:38 | 1221 aa | MW at 139825.2 kD |

| | |
|---|---|
| NOV12a, | MFSGTLGKNMKTQPPLSRMNREELEDSFFRLREDHMLVKELSWKQQDEIKRLRTTLLR |
| CG57819-01 Protein | |
| | LTAAGRDLRVAEEAAPLSETARRGQKAGWRQRLSMHQRPQMHRLQGHFHCVGPASPRR |
| | AQPRVQVGHRQLHTAGAPVPEKPKRGRDRLSYTAPPSFKEHATNENRGEVASKPSELA |
| | HIMASNTMQVEEPPKSPEKMWPKDENFEQRSSLECAQKAAELRASIKEKVELIRLKKL |
| | LHERNASLVMTKAQLTEVQEVSCHLLTQNQGILSAAHEALLKQVNELRAELKEESKKA |
| | VSLKSQLEDVSILQMTLKEFQERVEDLEKERKLLNDNYDKLLESSDSSSQPHWSNELT |
| | AEQLQQQVSQLQDQLDAELEDKRKVLLELSREKAQNEDLKLEVTNILQKHKQEVELLQ |
| | NAATISQPPDRQSEPATHPAVLQENTQIQPSEPKNQEEKKLSQVLNELQVSHAETTLE |
| | LEKTRDMLILQRKINVCYQEELEAMMTKADNDNRDHKEKLERLTRLLDLKNNRIKQLE |
| | EQLKDVAYGTRPLSLCLETLPAHGDEDKVDISLLHQGENLFELHIHQAFLTSAALAQA |
| | GDTQPTTFCTYSFYDFETHCTPLSVGPQPLYDFTSQYVMETDSLFLHYLQEASARLDI |
| | HQAMASEHSTLAAGWICFDRVLETVEKVHGLATLIGAGGEEFGVLEYWMRLRFPIKPS |
| | LQACNKRKKAQVYLSTDVLGGRKAQEEEVRSESWEPQNELWIEITKCCGLRSRWLGTQ |
| | PSPYAVYRFFTFSDHDTAIIPASNNPYFRDQARFPVLVTSDLDHYLRREALSIHVFDD |
| | EDLEPGSYLGRARVPLLPLAKNESIKGDFNLTDPAEKPNGSIQVQLDWKFPYIPPESF |
| | LKPEAQTKGKDTKDSSKISSEEEKASFPSQDQMASPEVPIEAGQYRSKRKPPHGGERK |
| | EKEHQVVSYSRRKHGKRIGVQGKNRMEYLSLNILNGNTLKQVNYTEWKFSETNSFIGD |
| | GFKNQHEEEEMTLSHSALKQKEPLHPVNDKESSEQGSEVSEAQTTDSDDVIVPPMSQK |
| | YPKADSEKMCIEIVSLAFYPEAEVMSDENIKQVYVEYKFYDLPLSETETPVSLRKPRA |
| | GEEIHFHFSKVIDLDPQEQQGRRRFLFDMLNGQDPDQGQLKFTVVSDPLDEEKKECEE |
| | VGYAYLQLWQILESGRDILEQELDVVSPEDLATPIGRLKVSLQAAAVLHAIYKEMTED |
| | LFS |

Further analysis of the NOV12a protein yielded the following properties shown in Table 12B.

TABLE 12B
Protein Sequence Properties NOV12a

| | |
|---|---|
| PSort | 0.9600 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV12a protein against the Geneseq database, a proprietary database that contains sequences published in patents and patent publication, yielded several homologous proteins shown in Table 12C.

TABLE 12C
Geneseq Results for NOV12a

| | | | Identities/ | |
|---|---|---|---|---|
| | | NOV12a | Similarities | |
| | Protein/ | Residues/ | for the | |
| Geneseq | Organism/Length | Match | Matched | Expect |
| Identifier | [Patent #, Date] | Residues | Region | Value |
| AAM78558 | Human protein | 63 . . . 1219 | 400/1193 | e−172 |
| | SEQ ID NO | | (33%) | |
| | 1220—Homo | 47 . . . 1177 | 640/1193 | |
| | sapiens, 1179 aa. | | (53%) | |
| | [WO200157190- | | | |
| | A2, Aug. 9, | | | |
| | 2001] | | | |
| AAM79542 | Human protein | 63 . . . 1219 | 400/1193 | e−172 |
| | SEQ ID NO | | (33%) | |
| | 3188—Homo | 28 . . . 1158 | 640/1193 | |
| | sapiens, 1160 aa. | | (53%) | |
| | [WO200157190- | | | |
| | A2, Aug. 9, | | | |
| | 2001] | | | |
| AAM41414 | Human poly- | 63 . . . 1219 | 400/1193 | e−172 |
| | peptide SEQ ID | | (33%) | |
| | NO 6345—Homo | 28 . . . 1158 | 640/1193 | |
| | sapiens, 1160 aa. | | (53%) | |
| | [WO200153312- | | | |
| | A1, Jul. 26, 2001] | | | |
| AAM39628 | Human poly- | 118 . . . 1219 | 390/1138 | e−171 |
| | peptide SEQ ID | | (34%) | |
| | NO 2773—Homo | 47 . . . 1126 | 623/1138 | |
| | sapiens, 1128 aa. | | (54%) | |
| | [WO200153312- | | | |
| | A1, Jul. 26, 2001] | | | |
| AAG75661 | Human colon | 445 . . . 523 | 40/79 | 1e−13 |
| | cancer antigen | | (50%) | |
| | protein SEQ ID | 33 . . . 111 | 56/79 | |
| | NO:6425—Homo | | (70%) | |
| | sapiens, 118 aa. | | | |
| | [WO200122920- | | | |
| | A2, Apr. 5, 2001] | | | |

In a BLAST search of public sequence databases, the NOV12a protein was found to have homology to the proteins shown in the BLASTP data in Table 12D.

TABLE 12D
Public BLASTP Results for NOV12a

| | | | Identities/ | |
|---|---|---|---|---|
| | | NOV12a | Similarities | |
| Protein | | Residues/ | for the | |
| Accession | Protein/ | Match | Matched | Expect |
| Number | Organism/Length | Residues | Portion | Value |
| Q96KN7 | RPGR- | 7 . . . 1221 | 1203/1258 | 0.0 |
| | INTERACTING | | (95%) | |
| | PROTEIN 1— | 29 . . . 1286 | 1207/1258 | |
| | Homo sapiens | | (95%) | |
| | (Human), 1286 aa. | | | |
| Q96QA8 | RPGR- | 7 . . . 1221 | 1203/1258 | 0.0 |
| | INTERACTING | | (95%) | |
| | PROTEIN 1— | 29 . . . 1286 | 1207/1258 | |
| | Homo sapiens | | (95%) | |
| | (Human), 1286 aa. | | | |
| Q9GLM3 | RPGR- | 1 . . . 1221 | 922/1234 | 0.0 |
| | INTERACTING | | (74%) | |
| | PROTEIN-1— | 1 . . . 1221 | 1031/1234 | |
| | Bos taurus | | (82%) | |
| | (Bovine), 1221 aa. | | | |
| Q9NR40 | RPGR- | 331 . . . 1221 | 883/902 | 0.0 |
| | INTERACTING | | (97%) | |
| | PROTEIN— | 1 . . . 902 | 888/902 | |
| | Homo sapiens | | (97%) | |
| | (Human), 902 aa. | | | |
| Q9HBK6 | RPGR- | 471 . . . 1221 | 742/763 | 0.0 |
| | INTERACTING | | (97%) | |
| | PROTEIN-1— | 1 . . . 762 | 746/763 | |
| | Homo sapiens | | (97%) | |
| | (Human), 762 aa. | | | |

PFam analysis predicts that the NOV12a protein contains the domains shown in the Table 12E.

TABLE 12E
Domain Analysis of NOV12a

| | NOV12a | Identities/Similarities | Expect |
|---|---|---|---|
| Pfam Domain | Match Region | for the Matched Region | Value |
| PFEMP: domain | 293 . . . 413 | 23/176 (13%) | 7.9 |
| 1 of 1 | | 82/176 (47%) | |
| C2: domain | 736 . . . 825 | 14/101 (14%) | 1.4 |
| 1 of 1 | | 54/101 (53%) | |

### Example 13

The NOV13 clone was analyzed, and the nucleotide and predicted polypeptide sequences are shown in Table 13A.

TABLE 13A
NOV13 Sequence Analysis

| | SEQ ID NO:39 | 678 bp | |
|---|---|---|---|
| NOV13a, | TGGGGCGGGAGGCATGGTCTCCACCTACCGGGTGGCCGTGCTGGGGGCGCGAGGTGTG | | |
| CG57789-01 DNA Sequence | | | |
| | GGCAAGAGTGCCATCGTGCGCCAGTTCTTGTACAACGAGTTCAGCGAGGTCTGCGTCC | | |
| | CCACCACCGCCCGCCGCCTTTACCTGCCTGCTGTCGTCATGAACGGCCACGTGCACGA | | |
| | CCTCCAGATCCTCGACTTTCCACCCATCAGCGCCTTCCCTGTCAATACGCTCCAGGAG | | |
| | TGGGCAGACACCTGCTGCAGGGGACTCCGGAGTGTCCACGCCTACATCCTGGTCTACG | | |
| | ACATCTGCTGCTTTGACAGCTTTGAGTACGTCAAGACCATCCGCCAGCAGATCCTGGA | | |
| | GACGAGGGTGATCGGAACCTCAGAGACGCCCATCATCATCGTGGGCAACAAGCGGGAC | | |
| | CTGCAGCGCGGACGCGTGATCCCGCGCTGGAACGTGTCGCACCTGGTACGCAAGACCT | | |
| | GGAAGTGCGGCTACGTGGAATGCTCGGCCAAGTACAACTGGCACATCCTGCTGCTCTT | | |
| | CAGCGAGCTGCTCAAGAGCGTCGGCTGCGCCCGTTGCAAGCACGTGCACGCTGCCCTG | | |
| | CGCTTCCAGGGCGCGCTGCGCCGCAACCGCTGCGCCATCATGTGACGCCTGCGCGCCC | | |
| | CTCGGGCTGCACCGGCACTGGCCGAGCGGAGGGCGGGGCC | | |
| | ORF Start: ATG at 14 | | ORF Stop: TGA at 623 |
| | SEQ ID NO:40 | 203 aa | MW at 23229.0 kD |
| NOV13a, | MVSTYRVAVLGARGVGKSAIVRQFLYNEFSEVCVPTTARRLYLPAVVMNGHVHDLQIL | | |
| CG57789-01 Protein Sequence | | | |
| | DFPPISAFPVNTLQEWADTCCRGLRSVHAYILVYDICCFDSFEYVKTIRQQILETRVI | | |
| | GTSETPIIIVGNKRDLQRGRVIPRWNVSHLVRKTWKCGYVECSAKYNWHILLLFSELL | | |
| | KSVGCARCKHVHAALRFQGALRRNRCAIM | | |
| | SEQ ID NO:41 | 682 bp | |
| NOV13b, | TGGGAGGCATGGTCTCCACCTACCGGGTGGCCGTGCTGGGGGCGCGAGGTGTGGGCAA | | |
| CG57789-02 DNA Sequence | | | |
| | GAGTGCCATCGTGCGCCAGTTCTTGTACAACGAGTTCAGCGAGGTCTGCGTCCCCACC | | |
| | ACCGCCCGCCGCCTTTACCTGCCTGCTGTCGTCATGAACGGCCACGTGCACGACCTCC | | |
| | AGATCCTCGACTTTCCACCCATCAGCGCCTTCCCTGTCAATACGCTCCAGGAGTGGGC | | |
| | AGACACCTGCTGCAGGGGACTCCGGAGTGTCCACGCCTACATCCTGGTCTACGACATC | | |
| | TGCTGCTTTGACAGCTTTGAGTACGTCAAGACCATCCGCCAGCAGATCCTGGAGACGA | | |
| | GGGTGATCGGAACCTCAGAGACGCCCATCATCATCGTGGGCAACAAGCGGGACCTGCA | | |
| | GCGCGGACGCGTGATCCCGCGCTGGAACGTGTCGCACCTGGTACGCAAGACCTGGAAG | | |
| | TGCGGCTACGTGGAATGCTCGGCCAAGTACAACTGGCACATCCTGCTGCTCTTCAGCG | | |
| | AGCTGCTCAAGAGCGTCGGCTGCGCCCGTTGCAAGCACGTGCACGCTGCCCTGCGCTT | | |
| | CCAGGGCGCGCTGCGCCGCAACCGCTGCGCCATCATGTGACGCCTGCGCGCCCCTCGG | | |
| | GCTGCACCGGCACTGGCCGAGCGGAGGGCACTGGCCGAGCGGAG | | |
| | ORF Start: ATG at 9 | | ORF Stop: TGA at 618 |
| | SEQ ID NO:42 | 203 aa | MW at 23229.0 kD |

| | |
|---|---|
| NOV13b, | MVSTYRVAVLGARGVGKSAIVRQFLYNEFSEVCVPTTARRLYLPAVVMNGHVHDLQIL |
| CG57789-02 Protein Sequence | |
| | DFPPISAFPVNTLQEWADTCCRGLRSVHAYILVYDICCFDSFEYVKTIRQQILETRVI |
| | GTSETPIIIVGNKRDLQRGRVIPRWNVSHLVRKTWKCGYVECSAKYNWHILLLFSELL |
| | KSVGCARCKHVHAALRFQGALRRNRCAIM |

Sequence comparison of the above protein sequences yields the following sequence relationships shown in Table 13B.

TABLE 13B
Comparison of NOV13a against NOV13b.

| Protein | NOV13a Residues/ | Identities/Similarities |
|---|---|---|
| Sequence | Match Residues | for the Matched Region |
| NOV13b | 1 . . . 203 | 203/203 (100%) |
| | 1 . . . 203 | 203/203 (100%) |

Further analysis of the NOV13a protein yielded the following properties shown in Table 13C.

TABLE 13C
Protein Sequence Properties NOV13a

| | |
|---|---|
| PSort | 0.6500 probability located in plasma membrane; 0.5064 |
| analysis: | probability located in mitochondrial matrix space; 0.3844 |
| | probability located in microbody (peroxisome); 0.2556 |
| | probability located in lysosome (lumen) |
| SignalP | No Known Signal Sequence Predicted |
| analysis: | |

A search of the NOV13a protein against the Geneseq database, a proprietary database that contains sequences published in patents and patent publication, yielded several homologous proteins shown in Table 13D.

TABLE 13D
Geneseq Results for NOV13a

| | | | Identities/ | |
|---|---|---|---|---|
| | | NOV13a | Similarities | |
| | Protein/ | Residues/ | for the | |
| Geneseq | Organism/Length | Match | Matched | Expect |
| Identifier | [Patent #, Date] | Residues | Region | Value |
| AAB42840 | Human ORFX | 1 . . . 136 | 136/136 | 2e−75 |
| | ORF2604 poly- | | (100%) | |
| | peptide sequence | 1 . . . 136 | 136/136 | |
| | SEQ ID NO:5208— | | (100%) | |
| | Homo sapiens, | | | |
| | 136 aa. | | | |
| | [WO200058473-A2, | | | |
| | Oct. 5, 2000] | | | |
| AAM41682 | Human polypeptide | 5 . . . 174 | 66/171 | 4e−18 |
| | SEQ ID NO 6613— | | (38%) | |
| | Homo sapiens, | 15 . . . 173 | 189/171 | |
| | 206 aa. | | (51%) | |
| | [WO200153312-A1, | | | |
| | Jul. 26, 2001] | | | |
| AAM39896 | Human polypeptide | 5 . . . 174 | 66/171 | 4e−18 |
| | SEQ ID NO 3041— | | (38%) | |
| | Homo sapiens, | 8 . . . 166 | 89/171 | |
| | 199 aa. | | (51%) | |
| | [WO200153312-A1, | | | |
| | Jul. 26, 2001] | | | |
| AAY99656 | Human GTPase | 5 . . . 173 | 59/179 | 3e−14 |
| | associated protein- | | (32%) | |
| | 7—Homo sapiens, | 25 . . . 191 | 87/179 | |
| | 281 aa. | | (47%) | |
| | WO200031263-A2, | | | |
| | Jun. 2, 2000] | | | |
| AAR05075 | RAP1A Gene | 5 . . . 177 | 57/175 | 5e-14 |
| | product | | (32%) | |
| | incorporating at | 4 . . . 165 | 90/175 | |
| | least one peptide | | (50%) | |
| | associated with ras | | | |
| | oncogene— | | | |
| | Synthetic, 184 aa. | | | |
| | [WO9000179-A, | | | |
| | Jan. 11, 1990] | | | |

In a BLAST search of public sequence databases, the NOV13a protein was found to have homology to the proteins shown in the BLASTP data in Table 13E.

TABLE 13E
Public BLASTP Results for NOV13a

| | | NOV13a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96S79 | RAS-LIKE | 1 . . . 203 | 203/203 (100%) | e−118 |
| | PROTEIN/ | 1 . . . 203 | 203/203 (100%) | |
| | VTS58635— | | | |
| | Homo sapiens | | | |
| | (Human), 203 aa. | | | |
| Q92737 | Ras-like protein | 1 . . . 203 | 105/204 (51%) | 3e−50 |
| | RRP22 (RAS- | 1 . . . 203 | 134/204 (65%) | |
| | related protein | | | |
| | on chromosome | | | |
| | 22)—Homo | | | |
| | sapiens (Human), | | | |
| | 203 aa. | | | |
| Q95KD9 | HYPO- | 5 . . . 174 | 66/171 (38%) | 1e−17 |
| | THETICAL | 8 . . . 166 | 89/171 (51%) | |
| | 22.5 KDA | | | |
| | PROTEIN— | | | |
| | Macaca | | | |
| | fascicularis | | | |
| | (Crab eating | | | |
| | macaque) | | | |
| | (Cynomolgus | | | |
| | monkey), 199 aa. | | | |
| Q96HU8 | SIMILAR TO | 5 . . . 174 | 66/171 (38%) | 1e−17 |
| | CG8500 GENE | 8 . . . 166 | 89/171 (51%) | |
| | PRODUCT— | | | |
| | Homo sapiens | | | |
| | (Human), 199 aa. | | | |
| Q9NF75 | EG:BACR37P7.8 | 5 . . . 174 | 61/174 (35%) | 4e−16 |
| | PROTEIN— | 48 . . . 210 | 88/174 (50%) | |
| | Drosophila | | | |
| | melanogaster | | | |
| | (Fruit fly), | | | |
| | 306 aa. | | | |

PFam analysis predicts that the NOV13a protein contains the domains shown in the Table 13F.

TABLE 13F
Domain Analysis of NOV13a

| | NOV13a | Identities/Similarities | Expect |
|---|---|---|---|
| Pfam Domain | Match Region | for the Matched Region | Value |
| Semialdhyde_dh: | 4 . . . 14 | 4/11 (36%) | 0.75 |
| domain 1 of 1 | | 11/11 (100%) | |
| ras: domain 1 of 1 | 6 . . . 203 | 56/224 (25%) | 1.2e−12 |
| | | 125/224 (56%) | |

### Example 14

The NOV14 clone was analyzed, and the nucleotide and predicted polypeptide sequences are shown in Table 14A.

TABLE 14A
NOV14 Sequence Analysis

| | SEQ ID NO:43 | 1790 bp | |
|---|---|---|---|
| NOV14a, | TCTCCCTCCCGCGCGATGGCCTCGGCGCTGAGCTATGTCTCCAAGTTCAAGTCCTTCG | | |
| CG57758-01 DNA Sequence | | | |
| | TGATCTTGTTCGTCACCCCGCTCCTGCTGCTGCCACTCGTCATTCTGATGCCCGCCAA | | |
| | GGTCAGTTGTGCCTACGTCATCATCCTCATGGCCATTTACTGGTGCACAGAAGTCATC | | |
| | CCTCTGGCTGTCACCTCTCTCATGCCTGTCTTGCTTTTCCCACTCTTCCAGATTCTGG | | |
| | ACTCCAGGCAGGTGTGTGTCCAGTACATGAAGGACACCAACATGCTGTTCCTGGGCGG | | |
| | CCTCATCGTGGCCGTGGCTGTGGAGCGCTGGAACCTGCACAAGAGGATCGCCCTGCGC | | |
| | ACGCTCCTCTGGGTGGGGGCCAAGCCTGCACGGCTGATGCTGGGCTTCATGGGCGTCA | | |
| | CAGCCCTCCTGTCCATGTGGATCAGTAACACGGCAACCACGGCCATGATGGTGCCCAT | | |
| | CGTGGAGGCCATATTGCAGCAGATGGAAGCCACAAGCGCAGCCACCGAGGCCGGCCTG | | |
| | GAGCTGGTGGACAAGGGCAAGGCCAAGGAGCTGCCAGGGAGTCAAGTGATTTTTGAAG | | |
| | GCCCCACTCTGGGGCAGCAGGAAGACCAAGAGCGGAAGAGGTTGTGTAAGGCCATGAC | | |
| | CCTGTGCATCTGCTACGCGGCCAGCATCGGGGGCACCGCCACCCTGACCGGGACGGGA | | |
| | CCCAACGTGGTGCTCCTGGGCCAGATGAACGAGTTGTTTCCTGACAGCAAGGACCTCG | | |
| | TGAACTTTGCTTCCTGGTTTGCATTTGCCTTTCCCAACATGCTGGTGATGCTGCTGTT | | |
| | CGCCTGGCTGTGGCTCCAGTTTGTTTACATGTTCTCCAGTTTTAAAAAGTCCTGGGGC | | |
| | TGCGGGCTAGAGAGCAAGAAAAACGAGAAGGCTGCCCTCAAGGTGCTGCAGGAGGAGT | | |
| | ACCGGAAGCTGGGGCCCTTGTCCTTCGCGGAGATCAACGTGCTGATCTGCTTCTTCCT | | |
| | GCTGGTCATCCTGTGGTTCTCCCGAGACCCCGGCTTCATGCCCGGCTGGCTGACTGTT | | |
| | GCCTGGGTGGAGGGTGAGACAAAGTATGTCTCCGATGCCACTGTGGCCATCTTTGTGG | | |
| | CCACCCTGCTATTCATTGTGCCTTCACAGAAGCCCAAGTTTAACTTCCGCAGCCAGAC | | |
| | TGAGGAAGGTAAGTCTCCTGTTCTGATCGCCCCCCCTCCCCTGCTGGATTGGAAGGTA | | |
| | ACCCAGGAGAAAGTGCCCTGGGGCATCGTGCTGCTACTAGGGGGCGGATTTGCTCTGG | | |
| | CTAAAGGATCCGAGGCCTCGGGGCTGTCCGTGTGGATGGGGAAGCAGATGGAGCCCTT | | |
| | GCACGCAGTGCCCCCGGCAGCCATCACCTTGATCTTGTCCTTGCTCGTTGCCGTGTTC | | |
| | ACTGAGTGCACAAGCAACGTGGCCACCACCACCTTGTTCCTGCCCATCTTTGCCTCCA | | |
| | TGTCTCGCTCCATCGGCCTCAATCCGCTGTACATCATGCTGCCCTGTACCCTGAGTGC | | |
| | CTCCTTTGCCTTCATGTTGCCTGTGGCCACCCCTCCAAATGCCATCGTGTTCACCTAT | | |
| | GGGCACCTCAAGGTTGCTGACATGGTGAAAACAGGAGTCATAATGAACATAATTGGAG | | |
| | TCTTCTGTGTGTTTTTGGCTGTCAACACCTGGGGACGGGCCATATTTGACTTGGATCA | | |
| | TTTCCCTGACTGGGCTAATGTGACACATATTGAGACTTAGGAAGAGCCACAAGACCAC | | |
| | ACACACAGCCCTTACCCTCCTCAGGACTACCGAACCTTCTGGCACACCTT | | |
| | ORF Start: ATG at 16 | | ORF Stop: TAG at 1720 |
| | SEQ ID NO:44 | 568 aa | MW at 62592.9 kD |
| NOV14a, | MASALSYVSKFKSFVILFVTPLLLLPLVILMPAKVSCAYVIILMAIYWCTEVIPLAVT | | |
| CG57758-01 Protein Sequence | | | |
| | SLMPVLLFPLFQILDSRQVCVQYMKDTNMLFLGGLIVAVAVERWNLHKRIALRTLLWV | | |
| | GAKPARLMLGFMGVTALLSMWISNTATTAMMVPIVEAILQQMEATSAATEAGLELVDK | | |
| | GKAKELPGSQVIFEGPTLGQQEDQERKRLCKAMTLCICYAASIGGTATLTGTGPNVVL | | |
| | LGQMNELFPDSKDLVNFASWFAFAFPNMLVMLLFAWLWLQFVYMFSSFKKSWGCGLES | | |
| | KKNEKAALKVLQEEYRKLGPLSFAEINVLICFFLLVILWFSRDPGFMPGWLTVAWVEG | | |
| | ETKYVSDATVAIFVATLLFIVPSQKPKFNFRSQTEEGKSPVLIAPPPLLDWKVTQEKV | | |
| | PWGIVLLLGGGFALAKGSEASGLSVWMGKQMEPLHAVPPAAITLILSLLVAVFTECTS | | |
| | NVATTTLFLPIFASMSRSIGLNPLYIMLPCTLSASFAFMLPVATPPNAIVFTYGHLKV | | |
| | ADMVKTGVIMNIIGVFCVFLAVNTWGRAIFDLDHFPDWANVTHIET | | |
| | SEQ ID NO:45 | 1899 bp | |
| NOV14b, | CGTCTCGCCCGCCAGTCTCCCTCCCGCGCGATGGCCTCGGCGCTGAGCTATGTCTCCA | | |
| CG57758-02 DNA Sequence | | | |
| | AGTTCAAGTCCTTCGTGATCTTGTTCGTCACCCCGCTCCTGCTGCTGCCACTCGTCAT | | |
| | TCTGATGCCCGCCAAGGTCAGTTGCTGTGCCTACGTCATCATCCTCATGGCCATTTAC | | |
| | TGGTGCACAGAAGTCATCCCTCTGGCTGTCACCTCTCTCATGCCTGTCTTGCTTTTCC | | |
| | CACTCTTCCAGATTCTGGACTCCAGGCAGGTGTGTGTCCAGTACATGAAGGACACCAA | | |
| | CATGCTGTTCCTGGGCGGCCTCATCGTGGCCGTGGCTGTGGAGCGCTGGAACCTGCAC | | |
| | AAGAGGATCGCCCTGCGCACGCTCCTCTGGGTGGGGGCCAAGCCTGCACGGCTGATGC | | |
| | TGGGCTTCATGGGCGTCACAGCCCTCCTGTCCATGTGGATCAGTAACACGGCAACCAC | | |
| | GGCCATGATGGTGCCCATCGTGGAGGCCATATTGCAGCAGATGGAAGCCACAAGCGCA | | |
| | GCCACCGAGGCCGGCCTGGAGGGACAAGGTACCACAATAAACAACCTGAATGCACTGG | | |
| | AGGATGATACAGTGAAAGCAGTACTAGGAGGAAAGTGTGTAGCTATAATAAGCACTTA | | |
| | CGTCAAAAAAGTAGAAAAACTTCAAATAAACAATCTAATGACACCTCTTAAAAAACTA | | |
| | GAAAAGCAAGAGCAACAGGACCTAGGGCCTGGCATCAGGCCTCAGGACTCTGCCCAGT | | |
| | GCCAGGAAGACCAAGAGCGGAAGAGGTTGTGTAAGGCCATGACCCTGTGCATCTGCTA | | |
| | CGCGGCCAGCATCGGGGGCACCGCCACCCTGACCGGGACGGGACCCAACGTGGTGCTC | | |
| | CTGGGCCAGATGAACGAGTTGTTTCCTGACAGCAAGGACCTCGTGAACTTTGCTTCCT | | |
| | GGTTTGCATTTGCCTTTCCCAACATGCTGGTGATGCTGCTGTTCGCCTGGCTGTGGCT | | |
| | CCAGTTTGTTTACATGTTCTCCAGTTTTAAAAAGTCCTGGGGCTGCGGGCTAGAGAGC | | |
| | AAGAAAAACGAGAAGGCTGCCCTCAAGGTGCTGCAGGAGGAGTACCGGAAGCTGGGGC | | |
| | CCTTGTCCTTCGCGGAGATCAACGTGCTGATCTGCTTCTTCCTGCTGGTCATCCTGTG | | |
| | GTTCTCCCGAGACCCCGGCTTCATGCCCGGCTGGCTGACTGTTGCCTGGGTGGAGGGT | | |
| | GAGACAAAGTCAGTCTCCGATGCCACTGTGGCCATCTTTGTGGCCACCCTGCTATTCA | | |
| | TTGTGCCTTCACAGAAGCCCAAGTTTAACTTCCGCAGCCAGACTGAGGAAGGTAAGTC | | |
| | TCCTGTTCTGATCGCCCCCCCTCCCCTGCTGGATTGGAAGGTAACCCAGGAGAAAGTG | | |
| | CCCTGGGGCATCGTGCTGCTACTAGGGGGCGGATTTGCTCTGGCTAAAGGATCCGAGG | | |
| | CCTCGGGGCTGTCCGTGTGGATGGGGAAGCAGATGGAGCCCTTGCACGCAGTGCCCCC | | |
| | GGCAGCCATCACCTTGATCTTGTCCTTGCTCGTTGCCGTGTTCACTGAGTGCACAAGC | | |
| | AACGTGGCCACCACCACCTTGTTCCTGCCCATCTTTGCCTCCATGTCTCGCTCCATCG | | |
| | GCCTCAATCCGCTGTACATCATGCTGCCCTGTACCCTGAGTGCCTCCTTTGCCTTCAT | | |
| | GTTGCCTGTGGCCACCCCTCCAAATGCCATCGTGTTCACCTATGGGCACCTCAAGGTT | | |
| | GCTGACATGGTAAAAACAGGAGTCATAATGAACATAATTGGAGTCTTCTGTGTGTTTT | | |
| | TGGCTGTCAACACCTGGGGACGGGCCATATTTGACTTGGATCATTTCCCTGACTGGGC | | |
| | TAATGTGACACATATTGAGACTTAGGAAGAGCCACAAGACCAC | | |
| | ORF Start: ATG at 31 | | ORF Stop: TAG at 1879 |
| | SEQ ID NO:46 | 616 aa | MW at 67816.9 kD |
| NOV14b, | MASALSYVSKFKSFVILFVTPLLLLPLVILMPAKVSCCAYVIILMAIYWCTEVIPLAV | | |
| CG57758-02 Protein Sequence | | | |
| | TSLMPVLLFPLFQILDSRQVCVQYMKDTNMLFLGGLIVAVAVERWNLHKRIALRTLLW | | |
| | VGAKPARLMLGFMGVTALLSMWISNTATTAMMVPIVEAILQQMEATSAATEAGLEGQG | | |
| | TTINNLNALEDDTVKAVLGGKCVAIISTYVKKVEKLQINNLMTPLKKLEKQEQQDLGP | | |
| | GIRPQDSAQCQEDQERKRLCKAMTLCICYAASIGGTATLTGTGPNVVLLGQMNELFPD | | |
| | SKDLVNFASWFAFAFPNMLVMLLFAWLWLQFVYMFSSFKKSWGCGLESKKNEKAALKV | | |
| | LQEEYRKLGPLSFAEINVLICFFLLVILWFSRDPGFMPGWLTVAWVEGETKSVSDATV | | |
| | AIFVATLLFIVPSQKPKFNFRSQTEEGKSPVLIAPPPLLDWKVTQEKVPWGIVLLLGG | | |
| | GFALAKGSEASGLSVWMGKQMEPLHAVPPAAITLILSLLVAVFTECTSNVATTTLFLP | | |
| | IFASMSRSIGLNPLYIMLPCTLSASFAFMLPVATPPNAIVFTYGHLKVADMVKTGVIM | | |
| | NIIGVFCVFLAVNTWGRAIFDLDHFPDWANVTHIET | | |
| | SEQ ID NO:47 | 1899 bp | |
| NOV14c, | CGTCTCGCCCGCCAGTCTCCCTCCCGCGCGATGGCCTCGGCGCTGAGCTATGTCTCCA | | |
| CG57758-03 DNA Sequence | | | |
| | AGTTCAAGTCCTTCGTGATCTTGTTCGTCACCCCGCTCCTGCTGCTGCCACTCGTCAT | | |
| | TCTGATGCCCGCCAAGGTCAGTTGCTGTGCCTACGTCATCATCCTCATGGCCATTTAC | | |
| | TGGTGCACAGAAGTCATCCCTCTGGCTGTCACCTCTCTCATGCCTGTCTTGCTTTTCC | | |
| | CACTCTTCCAGATTCTGGACTCCAGGCAGGTGTGTGTCCAGTACATGAAGGACACCAA | | |
| | CATGCTGTTCCTGGGCGGCCTCATCGTGGCCGTGGCTGTGGAGCGCTGGAACCTGCAC | | |
| | AAGAGGATCGCCCTGCGCACGCTCCTCTGGGTGGGGGCCAAGCCTGCACGGCTGATGC | | |
| | TGGGCTTCATGGGCGTCACAGCCCTCCTGTCCATGTGGATCAGTAACACGGCAACCAC | | |
| | GGCCATGATGGTGCCCATCGTGGAGGCCATATTGCAGCAGATGGAAGCCACAAGCGCA | | |
| | GCCACCGAGGCCGGCCTGGAGGGACAAGGTACCACAATAAACAACCTGAATGCACTGG | | |
| | AGGATGATACAGTGAAAGCAGTACTAGGAGGAAAGTGTGTAGCTATAATAAGCACTTA | | |
| | CGTCAAAAAAGTAGAAAAACTTCAAATAAACAATCTAATGACACCTCTTAAAAAACTA | | |
| | GAAAAGCAAGAGCAACAGGACCTAGGGCCTGGCATCAGGCCTCAGGACTCTGCCCAGT | | |
| | GCCAGGAAGACCAAGAGCGGAAGAGGTTGTGTAAGGCCATGACCCTGTGCATCTGCTA | | |
| | CGCGGCCAGCATCGGGGGCACCGCCACCCTGACCGGGACGGGACCCAACGTGGTGCTC | | |
| | CTGGGCCAGATGAACGAGTTGTTTCCTGACAGCAAGGACCTCGTGAACTTTGCTTCCT | | |
| | GGTTTGCATTTGCCTTTCCCAACATGCTGGTGATGCTGCTGTTCGCCTGGCTGTGGCT | | |
| | CCAGTTTGTTTACATGTTCTCCAGTTTTAAAAAGTCCTGGGGCTGCGGGCTAGAGAGC | | |
| | AAGAAAAACGAGAAGGCTGCCCTCAAGGTGCTGCAGGAGGAGTACCGGAAGCTGGGGC | | |
| | CCTTGTCCTTCGCGGAGATCAACGTGCTGATCTGCTTCTTCCTGCTGGTCATCCTGTG | | |
| | GTTCTCCCGAGACCCCGGCTTCATGCCCGGCTGGCTGACTGTTGCCTGGGTGGAGGGT | | |
| | GAGACAAAGTCAGTCTCCGATGCCACTGTGGCCATCTTTGTGGCCACCCTGCTATTCA | | |
| | TTGTGCCTTCACAGAAGCCCAAGTTTAACTTCCGCAGCCAGACTGAGGAAGGTAAGTC | | |
| | TCCTGTTCTGATCGCCCCCCCTCCCCTGCTGGATTGGAAGGTAACCCAGGAGAAAGTG | | |
| | CCCTGGGGCATCGTGCTGCTACTAGGGGGCGGATTTGCTCTGGCTAAAGGATCCGAGG | | |
| | CCTCGGGGCTGTCCGTGTGGATGGGGAAGCAGATGGAGCCCTTGCACGCAGTGCCCCC | | |
| | GGCAGCCATCACCTTGATCTTGTCCTTGCTCGTTGCCGTGTTCACTGAGTGCACAAGC | | |
| | AACGTGGCCACCACCACCTTGTTCCTGCCCATCTTTGCCTCCATGTCTCGCTCCATCG | | |
| | GCCTCAATCCGCTGTACATCATGCTGCCCTGTACCCTGAGTGCCTCCTTTGCCTTCAT | | |
| | GTTGCCTGTGGCCACCCCTCCAAATGCCATCGTGTTCACCTATGGGCACCTCAAGGTT | | |
| | GCTGACATGGTAAAAACAGGAGTCATAATGAACATAATTGGAGTCTTCTGTGTGTTTT | | |
| | TGGCTGTCAACACCTGGGGACGGGCCATATTTGACTTGGATCATTTCCCTGACTGGGC | | |
| | TAATGTGACACATATTGAGACTTAGGAAGAGCCACAAGACCAC | | |
| | ORF Start: ATG at 31 | | ORF Stop: TAG at 1879 |
| | SEQ ID NO:48 | 616 aa | MW at 67816.9 kD |
| NOV14c, | MASALSYVSKFKSFVILFVTPLLLLPLVILMPAKVSCCAYVIILMAIYWCTEVIPLAV | | |
| CG57758-03 Protein Sequence | | | |
| | TSLMPVLLFPLFQILDSRQVCVQYMKDTNMLFLGGLIVAVAVERWNLHKRIALRTLLW | | |
| | VGAKPARLMLGFMGVTALLSMWISNTATTAMMVPIVEAILQQMEATSAATEAGLEGQG | | |
| | TTINNLNALEDDTVKAVLGGKCVAIISTYVKKVEKLQINNLMTPLKKLEKQEQQDLGP | | |
| | GIRPQDSAQCQEDQERKRLCKAMTLCICYAASIGGTATLTGTGPNVVLLGQMNELFPD | | |
| | SKDLVNFASWFAFAFPNMLVMLLFAWLWLQFVYMFSSFKKSWGCGLESKKNEKAALKV | | |
| | LQEEYRKLGPLSFAEINVLICFFLLVILWFSRDPGFMPGWLTVAWVEGETKSVSDATV | | |
| | AIFVATLLFIVPSQKPKFNFRSQTEEGKSPVLIAPPPLLDWKVTQEKVPWGIVLLLGG | | |
| | GFALAKGSEASGLSVWMGKQMEPLHAVPPAAITLILSLLVAVFTECTSNVATTTLFLP | | |
| | IFASMSRSIGLNPLYIMLPCTLSASFAFMLPVATPPNAIVFTYGHLKVADMVKTGVIM | | |
| | NIIGVFCVFLAVNTWGRAIFDLDHFPDWANVTHIET | | |
| | SEQ ID NO:49 | 1606 bp | |
| NOV14d, | GATGGCCTCGGCGCTGAGCTATGTCTCCAAGTTCAAGTCCTTCGTGATCTTGTTCGTC | | |
| CG57758-04 DNA Sequence | | | |
| | ACCCCGCTCCTGCTGCTGCCACTCGTCATTCTGATGCCCGCCAAGTTTGTCAGGTGTG | | |
| | CCTACGTCATCATCCTCATGGCCATTTACTGGTGCACAGAAGTCATCCCTCTGGCTGT | | |
| | CACCTCTCTCATGCCTGTCTTGCTTTTCCCACTCTTCCAGATTCTGGACTCCAGGCAG | | |
| | GTGTGTGTCCAGTACATGAAGGACACCAACATGCTGTTCCTGGGCGGCCTCATCGTGG | | |
| | CCGTGGCTGTGGAGCGCTGGAACCTGCACAAGAGGATCGCCCTGCGCACGCTCCTCTG | | |
| | GGTGGGGGCCAAGCCTGCACGGCTGATGCTGGGCTTCATGGGCGTCACAGCCCTCCTG | | |
| | TCCATGTGGATCAGTAACACGGCAACCACGGCCATGATGGTGCCCATCGTGGAGGCCA | | |
| | TATTGCAGCAGATGGAAGCCACAAGCGCAGCCACCGAGGCCGGCCTGGAGCTGGTGGA | | |
| | CAAGGGCAAGGCCAAGGAGCTGCCAGGGAGTCAAGTGATTTTTGAAGGCCCCACTCTG | | |
| | GGGCAGCAGGAAGACCAAGAGCGGAAGAGGTTGTGTAAGGCCATGACCCTGTGCATCT | | |
| | GCTACGCGGCCAGCATCGGGGGCACCGCCACCCTGACCGGGACGGGACCCAACGTGGT | | |
| | GCTCCTGGGCCAGATGAACGAGTTGTTTCCTGACAGCAAGGACCTCGTGAACTTTGCT | | |
| | TCCTGGTTTGCATTTGCCTTTCCCAACATGCTGGTGATGCTGCTGTTCGCCTGGCTGT | | |
| | GGCTCCAGTTTGTTTACATGAGATTCAATTTTAAAAAGTCCTGGGGCTGCGGGCTAGA | | |
| | GAGCAAGAAAAACGAGAAGGCTGCCCTCAAGGTGCTGCAGGAGGAGTACCGGAAGTTG | | |
| | GGGCCCTTGTCCTTCGCGGAGATCAACGTGCTGATCTGCTTCTTCCTGCTGGTCATCC | | |
| | TGTGGTTCTCCCGAGACCCCGGCTTCATGCCCGGCTGGCTGACTGTTGCCTGGGTGGA | | |
| | GGGTGAGACAAAGTATGTCTCCGATGCCACTGTGGCCATCTTTGTGGCCACCCTGCTA | | |
| | TTCATTGTGCCTTCACAGAAGCCCAAGTTTAACTTCCGCAGCCAGACTGAGGAAGAAA | | |
| | GGAAAACTCCATTTTATCCCCCTCCCCTGCTGGATTGGAAGGTAACCCAGGAGAAAGT | | |
| | GCCCTGGGGCATCGTGCTGCTACTAGGGGGCGGATTTGCTCTGGCTAAAGGATCCGAG | | |
| | GCCTCGGGGCTGTCCGTGTGGATGGGGAAGCAGATGGAGCCCTTGCACGCAGTGCCCC | | |
| | CGGCAGCCATCACCTTGATCTTGTCCTTGCTCGTTGCCGTGTTCACTGAGTGCACAAG | | |
| | CAACGTGGCCACCACCACCTTGTTCCTGCCCATCTTTGCCTCCATGGTGAAAACAGGA | | |
| | GTCATAATGAACATAATTGGAGTCTTCTGTGTGTTTTTGGCTGTCAACACCTGGGGAC | | |
| | GGGCCATATTTGACTTGGATCATTTCCCTGACTGGGCTAATGTGACACATATTGAGAC | | |
| | TTAGGAAGAGCCACAAGACCACACACATAGCCCTTACCCT | | |
| | ORF Start: ATG at 2 | | ORF Stop: TAG at 1568 |
| | SEQ ID NO:50 | 522 aa | MW at 58109.6 kD |
| NOV14d, | MASALSYVSKFKSFVILFVTPLLLLPLVILMPAKFVRCAYVIILMAIYWCTEVIPLAV | | |
| CG57758-04 Protein Sequence | | | |
| | TSLMPVLLFPLFQILDSRQVCVQYMKDTNMLFLGGLIVAVAVERWNLHKRIALRTLLW | | |
| | VGAKPARLMLGFMGVTALLSMWISNTATTAMMVPIVEAILQQMEATSAATEAGLELVD | | |
| | KGKAKELPGSQVIFEGPTLGQQEDQERKRLCKAMTLCICYAASIGGTATLTGTGPNVV | | |
| | LLGQMNELFPDSKDLVNFASWFAFAFPNMLVMLLFAWLWLQFVYMRFNFKKSWGCGLE | | |
| | SKKNEKAALKVLQEEYRKLGPLSFAEINVLICFFLLVILWFSRDPGFMPGWLTVAWVE | | |
| | GETKYVSDATVAIFVATLLFIVPSQKPKFNFRSQTEEERKTPFYPPPLLDWKVTQEKV | | |
| | PWGIVLLLGGGFALAKGSEASGLSVWMGKQMEPLHAVPPAAITLILSLLVAVFTECTS | | |
| | NVATTTLFLPIFASMVKTGVIMNIIGVFCVFLAVNTWGRAIFDLDHFPDWANVTHIET | | |
| | SEQ ID NO:51 | 1781 BP | |
| NOV14e, | GATGGCCTCGGCGCTGAGCTATGTCTCCAAGTTCAAGTCCTTCGTGATCTTGTTCGTC | | |
| CG57758-05 DNA Sequence | | | |
| | ACCCCGCTCCTGCTGCTGCCACTCGTCATTCTGATGCCCGCCAAGTTTGTCAGGTGTG | | |
| | CCTACGTCATCATCCTCATGGCCATTTACTGGTGCACAGAAGTCATCCCTCTGGCTGT | | |
| | CACCTCTCTCATGCCTGTCTTGCTTTTCCCACTCTTCCAGATTCTGGACTCCAGGCAG | | |
| | GTGTGTGTCCAGTACATGAAGGACACCAACATGCTGTTCCTGGGCGGCCTCATCGTGG | | |
| | CCGTGGCTGTGGAGCGCTGGAACCTGCACAAGAGGATCGCCCTGCGCACGCTCCTCTG | | |
| | GGTGGGGGCCAAGCCTGCACGGCTGATGCTGGGCTTCATGGGCGTCACAGCCCTCCTG | | |
| | TCCATGTGGATCAGTAACACGGCAACCACGGCCATGATGGTGCCCATCGTGGAGGCCA | | |
| | TATTGCAGCAGATGGAAGCCACAAGCGCAGCCACCGAGGCCGGCCTGGAGCTGGTGGA | | |
| | CAAGGGCAAGGCCAAGGAGCTGCCAGGGAGTCAAGTGATTTTTGAAGGCCCCACTCTG | | |
| | GGGCAGCAGGAAGACCAAGAGCGGAAGAGGTTGTGTAAGGCCATGACCCTGTGCATCT | | |
| | GCTACGCGGCCAGCATCGGGGGCACCGCCACCCTGACCGGGACGGGACCCAACGTGGT | | |
| | GCTCCTGGGCCAGATGAACGAGTTGTTTCCTGACAGCAAGGACCTCGTGAACTTTGCT | | |
| | TCCTGGTTTGCATTTGCCTTTCCCAACATGCTGGTGATGCTGCTGTTCGCCTGGCTGT | | |
| | GGCTCCAGTTTGTTTACATGAGATTCAATTTTAAAAAGTCCTGGGGCTGCGGGCTAGA | | |
| | GAGCAAGAAAAACGAGAAGGCTGCCCTCAAGGTGCTGCAGGAGGAGTACCGGAAGTTG | | |
| | GGGCCCTTGTCCTTCGCGGAGATCAACGTGCTGATCTGCTTCTTCCTGCTGGTCATCC | | |
| | TGTGGTTCTCCCGAGACCCCGGCTTCATGCCCGGCTGGCTGACTGTTGCCTGGGTGGA | | |
| | GGGTGAGACAAAGTATGTCTCCGATGCCACTGTGGCCATCTTTGTGGCCACCCTGCTA | | |
| | TTCATTGTGCCTTCACAGAAGCCCAAGTTTAACTTCCGCAGCCAGACTGAGGAAGAAA | | |
| | GGAAAACTCCATTTTATCCCCCTCCCCTGCTGGATTGGAAGGTAACCCAGGAGAAAGT | | |
| | GCCCTGGGGCATCGTGCTGCTACTAGGGGGCGGATTTGCTCTGGCTAAAGGATCCGAG | | |
| | GCCTCGGGGCTGTCCGTGTGGATGGGGAAGCAGATGGAGCCCTTGCACGCAGTGCCCC | | |
| | CGGCAGCCATCACCTTGATCTTGTCCTTGCTCGTTGCCGTGTTCACTGAGTGCACAAG | | |
| | CAACGTGGCCACCACCACCTTGTTCCTGCCCATCTTTGCCTCCATGAATCACGTCCCC | | |
| | AAGAGCTTCTGTGTTCTGTACGGTGATGTTGCAGTGCTGTCTTTCCGCAGTCTCGCTC | | |
| | CATCGGCCTCAATCCGCTGTACATCATGCTGCCCTGTACCCTGAGTGCCTCCTTTGCC | | |
| | TTCATGTTGCCTGTGGCCACCCCTCCAAATGCCATCGTGTTCACCTATGGGCACCTCA | | |
| | AGGTTGCTGACATGGTGAAAACAGGAGTCATAATGAACATAATTGGAGTCTTCTGTGT | | |
| | GTTTTTGGCTGTCAACACCTGGGGACGGGCCATATTTGACTTGGATCATTTCCCTGAC | | |
| | TGGGCTAATGTGACACATATTGAGACTTAGGAAGAGCCACA | | |
| | ORF Start: ATG at 2 | | ORF Stop: TGA at 1550 |
| | SEQ ID NO:52 | 516 aa | MW at 57173.5 kD |

| | |
|---|---|
| NOV14e, | MASALSYVSKFKSFVILFVTPLLLLPLVILMPAKFVRCAYVILMAIYWCTEVIPLAV |
| CG57758-05 Protein Sequence | |
| | TSLMPVLLFPLFQILDSRQVCVQYMKDTNMLFLGGLIVAVAVERWNLHKRIALRTLLW |
| | VGAKPARLMLGFMGVTALLSMWISNTATTAMMVPIVEAILQQMEATSAATEAGLELVD |
| | KGKAKELPGSQVIFEGPTLGQQEDQERKRLCKAMTLCICYAASIGGTATLTGTGPNVV |
| | LLGQMNELFPDSKDLVNFASWFAFAFPNMLVMLLFAWLWLQFVYMRFNFKKSWGCGLE |
| | SKKNEKAALEVLQEEYRKLGPLSFAEINVLICFFLLVILWFSRDPGFMPGWLTVAWVE |
| | GETKYVSDATVAIFVATLLFIVPSQKPKFNFRSQTEEERKTPFYPPPLLDWKVTQEKV |
| | PWGIVLLLGGGFALAKGSEASGLSVWMGKQMEPLHAVPPAAITLILSLLVAVFTECTS |
| | NVATTTLFLPIFAS |

Sequence comparison of the above protein sequences yields the following sequence relationships shown in Table 14B.

TABLE 14B
Comparison of NOV14a against NOV14b through NOV14e.

| Protein | NOV14a Residues/ | Identities/Similarities |
|---|---|---|
| Sequence | Match Residues | for the Matched Region |
| NOV14b | 1 . . . 568 | 519/616 (84%) |
| | 1 . . . 616 | 524/616 (84%) |
| NOV14c | 1 . . . 568 | 519/616 (84%) |
| | 1 . . . 616 | 524/616 (84%) |
| NOV14d | 1 . . . 568 | 483/570 (84%) |
| | 1 . . . 522 | 485/570 (84%) |
| NOV14e | 1 . . . 480 | 440/482 (91%) |
| | 1 . . . 480 | 443/482 (91%) |

Further analysis of the NOV14a protein yielded the following properties shown in Table 14C.

TABLE 14C
Protein Sequence Properties NOV14a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Likely cleavage site between residues 38 and 39 |
| analysis: | |

A search of the NOV14a protein against the Geneseq database, a proprietary database that contains sequences published in patents and patent publication, yielded several homologous proteins shown in Table 14D.

TABLE 14D
Geneseq Results for NOV14a

| | | | Identities/ | |
|---|---|---|---|---|
| | | NOV14a | Similarities | |
| | Protein/ | Residues/ | for the | |
| Geneseq | Organism/Length | Match | Matched | Expect |
| Identifier | [Patent #, Date] | Residues | Region | Value |
| AAB23625 | Human secreted | 10 . . . 566 | 256/623 | e−137 |
| | protein SEQ ID NO: | | (41%) | |
| | 50—Homo sapiens, | 9 . . . 623 | 386/623 | |
| | 627 aa. | | (61%) | |
| | [WO200049134-A1, | | | |
| | Aug. 24, 2000] | | | |
| AAB36158 | Novel human | 10 . . . 566 | 256/623 | e−137 |
| | transporter protein | | (41%) | |
| | SEQ ID NO: 2— | 9 . . . 623 | 386/623 | |
| | Homo sapiens, | | (61%) | |
| | 627 aa. | | | |
| | [WO200065055-A2, | | | |
| | Nov. 2, 2000] | | | |
| AAB42213 | Human ORFX | 10 . . . 566 | 256/623 | e−136 |
| | ORF1977 poly- | | (41%) | |
| | peptide sequence | 9 . . . 623 | 386/623 | |
| | SEQ ID NO: | | (61%) | |
| | 3954—Homo | | | |
| | sapiens, 627 aa. | | | |
| | [WO200058473-A2, | | | |
| | Oct. 5, 2000] | | | |
| AAB36164 | Novel human | 10 . . . 566 | 252/623 | e−136 |
| | transporter protein | | (40%) | |
| | SEQ ID NO: 14— | 9 . . . 622 | 382/623 | |
| | Homo sapiens, | | (60%) | |
| | 626 aa. | | | |
| | [WO200065055-A2, | | | |
| | Nov. 2, 2000] | | | |
| AAB36159 | Novel human | 10 . . . 566 | 256/623 | e−136 |
| | transporter protein | | (41%) | |
| | SEQ ID NO: 4— | 9 . . . 623 | 385/623 | |
| | Homo sapiens, | | (61%) | |
| | 627 aa. | | | |
| | [WO200065055-A2, | | | |
| | Nov. 2, 2000] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| O57661 | INTESTINAL | 1 . . . 564 | 336/619 (54%) | 0.0 |
| | SODIUM/ | 1 . . . 619 | 444/619 (71%) | |
| | LITHIUM | | | |
| | DEPENDENT DI- | | | |
| | CARBOXYLATE | | | |
| | TRANSPORTER | | | |
| | (NA(+)/DI- | | | |
| | CARBOXYLATE | | | |
| | COTRANS- | | | |
| | PORTER)— | | | |
| | Xenopus laevis | | | |
| | (African clawed | | | |
| | frog), 622 aa. | | | |
| Q9ES88 | NA/DICARB- | 1 . . . 561 | 311/572 (54%) | e−179 |
| | OXYLATE CO- | 1 . . . 567 | 421/572 (73%) | |
| | TRANSPORTER | | | |
| | (SOLUTE | | | |
| | CARRIER | | | |
| | FAMILY 13 | | | |
| | (SODIUM- | | | |
| | TRANSPORTER), | | | |
| | MEMBER 2)— | | | |
| | Mus musculus | | | |
| | (Mouse), 586 aa. | | | |
| O35055 | SODIUM/DI- | 1 . . . 562 | 311/572 (54%) | e−179 |
| | CARBOXYLATE | 1 . . . 568 | 419/572 (72%) | |
| | COTRANS- | | | |
| | PORTER 1 | | | |
| | (NA(+)/DI- | | | |
| | CARBOXYLATE | | | |
| | COTRANS- | | | |
| | PORTER 1) | | | |
| | (KIDNEY DI- | | | |
| | CARBOXYLATE | | | |
| | TRANSPORTER) | | | |
| | (SDCT1) | | | |
| | (ORGANIC ANION | | | |
| | TRANSPORTER 1) | | | |
| | (OAT1)—Rattus | | | |
| | norvegicus (Rat), | | | |
| | 587 aa. | | | |
| Q13183 | Renal sodium/ | 1 . . . 561 | 318/581 (54%) | e−179 |
| | dicarboxylate | 1 . . . 572 | 428/581 (72%) | |
| | cotransporter | | | |
| | (Na(+)/ | | | |
| | dicarboxylate | | | |
| | cotransporter)— | | | |
| | Homo sapiens | | | |
| | (Human), 592 aa. | | | |
| Q28615 | Renal sodium/ | 1 . . . 562 | 300/586 (51%) | e−172 |
| | dicarboxylate | 1 . . . 576 | 418/586 (71%) | |
| | cotransporter | | | |
| | (Na(+)/ | | | |
| | dicarboxylate | | | |
| | cotransporter)— | | | |
| | Oryctolagus | | | |
| | cuniculus (Rabbit), | | | |
| | 593 aa. | | | |

PFam analysis predicts that the NOV14a protein contains the domains shown in the Table 14F.

TABLE 14F
Domain Analysis of NOV14a

| | NOV14a | Identities/Similarities | Expect |
|---|---|---|---|
| Pfam Domain | Match Region | for the Matched Region | Value |
| Na_sulph_symp: | 6 . . . 554 | 163/604 (27%) | 8.3e−140 |
| domain 1 of 1 | | 424/604 (70%) | |

### Example 15

The NOV15 clone was analyzed, and the nucleotide and predicted polypeptide sequences are shown in Table 15A.

TABLE 15A
NOV15 Sequence Analysis

| | SEQ ID NO:53 | 1547 bp | |
|---|---|---|---|
| NOV15a, | AACCCCCTTGACTGAAGCAATGGAGGGGGGTCCAGCTGTCTGCTGCCAGGATCCTCGG | | |
| CG57732-01 DNA Sequence | | | |
| | GCAGAGCTGGTAGAACGGGTGGCAGCCATCGATGTGACTCACTTGGAGGAGGCAGATG | | |
| | GTGGCCCAGAGCCTACTAGAAACGGTGTGGACCCCCCACCACGGGCCAGAGCTGCCTC | | |
| | TGTGATCCCTGGCAGTACTTCAAGACTGCTCCCAGCCCGGCCTAGCCTCTCAGCCAGG | | |
| | AAGCTTTCCCTACAGGAGCGGCCAGCAGGAAGCTATCTGGAGGCGCAGGCTGGGCCTT | | |
| | ATGCCACGGGGCCTGCCAGCCACATCTCCCCCCGGGCCTGGCGGAGGCCCACCATCGA | | |
| | GTCCCACCACGTGGCCATCTCAGATGCAGAGGACTGCGTGCAGCTGAACCAGTACAAG | | |
| | CTGCAGAGTGAGATTGGCAAGGGTGCCTACGGTGTGGTGAGGCTGGCCTACAACGAAA | | |
| | GTGAAGACAGACACTATGCAATGAAAGTCCTTTCCAAAAAGAAGTTACTGAAGCAGTA | | |
| | TGGCTTTCCACGTCGCCCTCCCCCGAGAGGGTCCCAGGCTGCCCAGGGAGGACCAGCC | | |
| | AAGCAGCTGCTGCCCCTGGAGCGGGTGTACCAGGAGATTGCCATCCTGAAGAAGCTGG | | |
| | ACCACGTGAATGTGGTCAAACTGATCGAGGTACTGGATGACCCAGCTGAGGACAACCT | | |
| | CTATTTGCCCCGCATCCTTCTCCATAGGCCCGTCATGGAAGTGCCCTGTGACAAGCCC | | |
| | TTCTCGGAGGAGCAAGCTCGCCTCTACCTGCGGGACGTCATCCTGGGCCTCGAGTACG | | |
| | TGCACTGCCAGAAGATCGTCCACAGGGACATCAAGCCATCCAACCTGCTCCTGGGGGA | | |
| | TGATGGGCACGTGAAGATCGCCGACTTTGGCGTCAGCAACCAGTTTGAGGGGAACGAC | | |
| | GCTCAGCTGTCCAGCACGGCGGGAACCCCAGCATTCATGGCCCCCGAGGCCATTTCTG | | |
| | ATTCCGGCCAGAGCTTCAGTGGGAAGTTGGATGTATGGGCCACTGGCGTCACGTTGTA | | |
| | CTGCTTTGTCTATGGGAAGTGCCCATTCATCGACGATTTCATCCTGGCCCTCCACAGG | | |
| | AAGATCAAGAATGAGCCCGTGGTGTTTCCTGAGGAGCCAGAAATCAGCGAGGAGCTCA | | |
| | AGGACCTGATCCTGAAGATGTTAGACAAGAATCCCGAGACGAGAATTGGGGTGCCAGA | | |
| | CATCAAGTTGCACCCTTGGGTGACCAAGAACGGGGAGGAGCCCCTTCCTTCGGAGGAG | | |
| | GAGCACTGCAGCGTGGTGGAGGTGACAGAGGAGGAGGTTAAGAACTCAGTCAGGCTCA | | |
| | TCCCCAGCTGGACCACGGTGATCCTGGTGAAGTCCATGCTGAGGAAGCGTTCCTTTGG | | |
| | GAACCCGTTTGAGCCCCAAGCACGGAGGGAAGAGCGATCCATGTCTGCTCCAGGAAAC | | |
| | CTACTGGTGAAAGAAGGGTTTGGTGAAGGGGGCAAGAGCCCAGAGCTCCCCGGCGTCC | | |
| | AGGAAGACGAGGCTGCATCCTGAGCCCCTGCATGCACCC | | |
| | ORF Start: ATG at 20 | | ORF Stop: TGA at 1529 |
| | SEQ ID NO:54 | 503 aa | MW at 55606.7 kD |
| NOV15a, | MEGGPAVCCQDPRAELVERVAAIDVTHLEEADGGPEPTRNGVDPPPRARAASVIPGST | | |
| CG57732-01 Protein Sequence | | | |
| | SRLLPARPSLSARKLSLQERPAGSYLEAQAGPYATGPASHISPRAWRRPTIESHHVAI | | |
| | SDAEDCVQLNQYKLQSEIGKGAYGVVRLAYNESEDRHYAMKVLSKKKLLKQYGFPRRP | | |
| | PPRGSQAAQGGPAKQLLPLERVYQEIAILKKLDHVNVVKLIEVLDDPAEDNLYLPRIL | | |
| | LERPVMEVPCDKPFSEEQARLYLRDVILGLEYVHCQKIVHRDIKPSNLLLGDDGHVKI | | |
| | ADFGVSNQFEGNDAQLSSTAGTPAFMAPEAISDSGQSFSGKLDVWATGVTLYCFVYGK | | |
| | CPFIDDFILALHRKIKNEPVVFPEEPEISEELKDLILKMLDKNPETRIGVPDIKLHPW | | |
| | VTKNGEEPLPSEEEHCSVVEVTEEEVKNSVRLIPSWTTVILVKSMLRKRSFGNPFEPQ | | |
| | ARREERSMSAPGNLLVKEGFGEGGKSPELPGVQEDEAAS | | |
| | SEQ ID NO:55 | 1611 bp | |
| NOV15b, | GCGCCCAGGTTCCCAACAAGGCTACGCAGAAGAACCCCCTTGACTGAAGCAATGGAGG | | |
| CG57732-02 DNA Sequence | | | |
| | GGGGTCCAGCTGTCTGCTGCCAGGATCCTCGGGCAGAGCTGGTAGAACGGGTGGCAGC | | |
| | CATCGATGTGACTCACTTGGAGGAGGCAGATGGTGGCCCAGAGCCTACTAGAAACGGT | | |
| | GTGGACCCCCCACCACGGGCCAGAGCTGCCTCTGTGATCCCTGGCAGTACTTTAAGAC | | |
| | TGCTCCCAGCCCGGCCTAGCCTCTCAGCCAGGAAGCTTTCCCTACAGGAGCGGCCAGC | | |
| | AGGAAGCTATCTGGAGGCGCAGGCTGGGCCTTATGCCACGGGGCCTGCCAGCCACATC | | |
| | TCCCCCCGGGCCTGGCGGAGGCCCACCATCGAGTCCCACCACGTGGCCATCTCAGATG | | |
| | CAGAGGACTGCGTGCAGCTGAACCAGTACAAGCTGCAGAGTGAGATTGGCAAGGGTGC | | |
| | CTACGGTGTGGTGAGGCTGGCCTACAACGAAAGTGAAGACAGACACTATGCAATGAAA | | |
| | GTCCTTTCCAAAAAGAAGTTACTGAAGCAGTATGGCTTTCCACGTCGCCCTCCCCCGA | | |
| | GAGGGTCCCAGGCTGCCCAGGGAGGACCAGCCAAGCAGCTGCTGCCCCTGGAGCGGGT | | |
| | GTACCAGGAGATTGCCATCCTGAAGAAGCTGGACCACGTGAATGTGGTCAAACTGATC | | |
| | GAGGTCCTGGATGACCCAGCTGAGGACAACCTCTATTTGGTGTTTGACCTCCTGAGAA | | |
| | AGGGGCCCGTCATGGAAGTGCCCTGTGACAAGTCCTTCTCGGAGGAGCAAGCTCGCCT | | |
| | CTACCTGCGGGACGTCATCCTGGGCCTCGAGTACTTGCACTGCCAGAAGATCSTCCAC | | |
| | AGGGACATCAAGCCATCCAACCTGCTCCTGGGGGATGATGGGCACGTGAAGATCGCCG | | |
| | ACTTTGGCGTCAGCAACCAGTTTGAGGGGAACGACGCTCAGCTGTCCAGCACGGCGGG | | |
| | AACCCCAGCATTCATGGCCCCCGAGGCCATTTCTGATTCCGGCCAGAGCTTCAGTGGG | | |
| | AAGGCCTTGGATGTATGGGCCACTGGCGTCACGCTGTACTGCTTTGTCTATGGGAAGT | | |
| | GCCCGTTCATCGACGATTTCATCCTGGCCCTCCACAGGAAGATCAAGAATGAGCCCGT | | |
| | GGTGTTTCCTGAGGGGCCAGAAATCAGCGAGGAGCTCAAGGACCTGATCCTGAAGATG | | |
| | ATCCTGGTGAAGTCCATGCTGAGGAAGCGTTCCTTTGGGAACCCGTTTGAGCCCCAAG | | |
| | CACGGAGGGAAGAGCGATCCATGTCTGCTCCAGGAAACCTACTGGTGAAAGAAGGGTT | | |
| | TGGTGAAGGGGGCAAGAGCCCAGAGCTCCCCGGCGTCCAGGAAGACGAGGCTGCATCC | | |
| | TGAGCCCCTGCATGCACCCAGGGCCACCCGGCAGCACACTCATCC | | |
| | ORF Start: ATG at 52 | | ORF Stop: TGA at 1567 |
| | SEQ ID NO:56 | 505 aa | MW at 55652.7 kD |
| NOV15b, | MEGGPAVDDQDPRAELVERVAAIDVTHLEEADGGPEPTRNGVDPPPRARAASVIPGST | | |
| CG57732-02 Protein Sequence | | | |
| | SRLLPARPSLSARKLSLQERPAGSYLEAQAGPYATGPASHISPRAWRRPTIESHHVAI | | |
| | SDAEDCVQLNQYKLQSEIGKGAYGVVRLAYNESEDRHYAMKVLSKKKLLKQYGFPRRP | | |
| | PPRGSQAAQGGPAEQLLPLERVYQEIAILKKLDHVNVVKLIEVLDDPAEDNLYLVFDL | | |
| | LRKGPVMEVPCDKSFSEEQARLYLRDVILGLEYLHCQKIVHRDIKPSNLLLGDDGHVK | | |
| | IADFGVSNQFEGNDAQLSSTAGTPAFMAPEAISDSGQSFSGKALDVWATGVTLYCFVY | | |
| | GKCPFIDDFILALHRKIKNEPVVFPEGPEISEELKDLILKMLDKNPETRIGVPDIKLH | | |
| | PWVTKNGEEPLPSEEEHCSVVEVTEEEVKNSVRLIPSWTTVILVKSMLRKRSFGNPPE | | |
| | PQARREERSMSAPGNLLVKEGFGEGGKSPELPGVQEDEAAS | | |
| | SEQ ID NO:57 | 1725 bp | |
| NOV15c, | GCGCCCAGGTTCCCAACAAGGCTACGCAGAAGAACCCCCTTGACTGAAGTAATGGAGG | | |
| CG57732-03 DNA Sequence | | | |
| | GGGGTCCAGCTGTCTGCTGCCAGGATCCTCGGGCAGAGCTGGTAGAACGGGTGGCAGC | | |
| | CATCGATGTGACTCACTTGGAGGAGGCAGATGGTGGCCCAGAGCCTACTAGAAACGGT | | |
| | GTGGACCCCCCACCACGGGCCAGAGCTGCCTCTGTGATCCCTGGCAGTACTTCAAGAC | | |
| | TGCTCCCAGCCCGGCCTAGCCTCTCAGCCAGGAAGCTTTCCCTACAGGAGCGGCCAGC | | |
| | AGGAAGCTATCTGGAGGCGCAGGCTGGGCCTTATGCCACGGGGCCTGCCAGCCACATC | | |
| | TCCCCCCGGGCCTGGCGGAGGCCCACCATCGAGTCCCACCACGTGGCCATCTCAGATG | | |
| | CAGAGGACTGCGTGCAGCTGAACCAGTACAAGCTGCAGAGTGAGATTGGCAAGGGTGC | | |
| | CTACGGTGTGGTGAGGCTGGCCTACAACGAAAGTGAAGACAGACACTATGCAATGAAA | | |
| | GTCCTTTCCAAAAAGAAGTTACTGAAGCAGTATGGCTTTCCACGTCGCCCTCCCCCGA | | |
| | GAGGGTCCCAGGCTGCCCAGGGAGGACCAGCCAAGCAGCTGCTGCCCCTGGAGCGGGT | | |
| | GTACCAGGAGATTGCCATCCTGAAGAAGCTGGACCACGTGAATGTGGTCAAACTGATC | | |
| | GAGGTCCTGGATGACCCGGCTGAGGACAACCTCTATTTGGCCCTGCAGAACCAGGCCC | | |
| | AGAATATCCAGTTAGATTCAACAAATATCGCCAAGTCCCACTCCCTGCTTCCCTCTGA | | |
| | GCAGCAAGACAGTGGATCCACGTGGGCTGCGCGCTCAGTGTTTGACCTCCTGAGAAAG | | |
| | GGGCCCGTCATGGAAGTGCCCTGTGACAAGCCCTTCTCGGAGGAGCAAGCTCGCCTCT | | |
| | ACCTGCGGGACGTCATCCTGGGCCTCGAGTACTTGCACTGCCAGAAGATCGTCCACAG | | |
| | GGACATCAAGCCATCCAACCTGCTCCTGGGGGATGATGGGCACGTGAAGATCGCCGAC | | |
| | TTTGGCGTCAGCAACCAGTTTGAGGGGAACGACGCTCAGCTGTCCAGCACGGCGGGAA | | |
| | CCCCAGCATTCATGGCCCCCGAGGCCATTTCTGATTCCGGCCAGAGCTTCAGTGGGAA | | |
| | GGCCTTGGATGTATGGGCCACTGGCGTCACGTTGTACTGCTTTGTCTATGGGAAGTGC | | |
| | CCGTTCATCGACGATTTCATCCTGGCCCTCCACAGGAAGACCAAGAATGAGCCCGTGG | | |
| | TGTTTCCTGAGGGGCCAGAAATCAGCGAGGAGCTCAAGGACCTGATCCTGAAGATGTT | | |
| | AGACAAGAATCCCGAGACGAGAATTGGGGTGCCAGACATCAAGTTGCACCCTTGGGTG | | |
| | ACCAAGAACGGGGAGGAGCCCCTTCCTTCGGAGGAGGAGCACTGCAGCGTGGTGGAGG | | |
| | TGACAGAGGAGGAGGTTAAGAACTCAGTCAGGCTCATCCCCAGCTGGACCACGGTGAT | | |
| | CCTGGTGAAGTCCATGCTGAGGAAGCGTTCCTTTGGGAACCCGTTTGAGCCCCAAGCA | | |
| | CGGAGGGAAGAGCGATCCATGTCTGCTCCAGGAAACCTACTGGTGAAAGAAGGGTTTG | | |
| | GTGAAGGGGGCAAGAGCCCAGAGCTCCCCGGCGTCCAGGAAGACGAGGCTGCATCCTG | | |
| | AGCCCCTGCATGCACCCAGGGCCACCCGGCAGCACACTCATCC | | |
| | ORF Start: ATG at 52 | | ORF Stop: TGA at 1681 |
| | SEQ ID NO:58 | 543 aa | MW at 59729.0 kD |

| | |
|---|---|
| NOV15c, | MEGGPAVCCQDPRAELVERVAAIDVTHLEEADGGPEPTRNGVDPPPRARAASVIPGST |
| CG57732-03 Protein Sequence | |
| | SRLLPARPSLSARKLSLQERPAGSYLEAQAGPYATGPASHISPRAWRRPTIESHHVAI |
| | SDAEDCVQLNQYKLQSEIGKGAYGVVRLAYNESEDRHYAMKVLSKKKLLKQYGFPRRP |
| | PPRGSQAAQGGPAKQLLPLERVYQEIAILKKLDHVNVVKLIEVLDDPAEDNLYLALQN |
| | QAQNIQLDSTNIAKSHSLLPSEQQDSGSTWAARSVFDLLRKGPVMEVPCDKPFSEEQA |
| | RLYLRDVILGLEYLHCQKIVHRDIKPSNLLLGDDGHVKIADFGVSNQFEGNDAQLSST |
| | AGTPAFMAPEAISDSGQSFSGKALDVWATGVTLYCFVYGKCPPIDDFILALHRKTKNE |
| | PVVFPEGPEISEELKDLILKMLDKNPETRIGVPDIKLHPWVTKNGEEPLPSEEEHCSY |
| | VEVTEEEVKNSVRLIPSWTTVILVKSMLRKRSFGNPFEPQARREERSMSAPGNLLVKE |
| | GFGEGGKSPELPGVQEDEAAS |

Sequence comparison of the above protein sequences yields the following sequence relationships shown in Table 15B.

TABLE 15B
Comparison of NOV15a against NOV15b through NOV15c.

| Protein | NOV15a Residues/ | Identities/Similarities |
|---|---|---|
| Sequence | Match Residues | for the Matched Region |
| NOV15b | 1 . . . 503 | 495/505 (98%) |
| | 1 . . . 505 | 497/505 (98%) |
| NOV15c | 1 . . . 503 | 492/543 (90%) |
| | 1 . . . 543 | 495/543 (90%) |

Further analysis of the NOV15a protein yielded the following properties shown in Table 15C.

TABLE 15C
Protein Sequence Properties NOV15a

| | |
|---|---|
| PSort | 0.7600 probability located in nucleus; 0.3000 probability located in |
| analysis: | microbody (peroxisome); 0.1000 probability located in mitochondrial |
| | matrix space; 0.1000 probability located in lysosome (lumen) |
| SignalP | No Known Signal Sequence Predicted |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | | Identities/ | |
|---|---|---|---|---|
| | | NOV15a | Similarities | |
| | Protein/ | Residues/ | for the | |
| Geneseq | Organism/Length | Match | Matched | Expect |
| Identifier | [Patent #, Date] | Residues | Region | Value |
| AAU03510 | Human protein | 1 . . . 503 | 496/513 | 0.0 |
| | kinase #10—Homo | | (96%) | |
| | sapiens, 513 aa. | 1 . . . 513 | 498/513 | |
| | [WO200138503-A2, | | (96%) | |
| | May 31, 2001] | | | |
| AAE04361 | Human kinase | 1 . . . 503 | 496/513 | 0.0 |
| | (PKIN)-2—Homo | | (96%) | |
| | sapiens, 513 aa. | 1 . . . 513 | 498/513 | |
| | [WO200146397-A2, | | (96%) | |
| | Jun. 28, 2001] | | | |
| AAY44239 | Human cell | 64 . . . 500 | 289/450 | e−165 |
| | signalling protein- | | (64%) | |
| | 2—Homo sapiens, | 90 . . . 538 | 367/450 | |
| | 540 aa. | | (81%) | |
| | [WO9958558-A2, | | | |
| | Nov. 18, 1999] | | | |
| AAM40450 | Human polypeptide | 64 . . . 482 | 283/432 | e−162 |
| | SEQ ID NO 5381— | | (65%) | |
| | Homo sapiens, | 128 . . . 558 | 356/432 | |
| | 680 aa. | | (81%) | |
| | [WO200153312-A1, | | | |
| | Jul. 26, 2001] | | | |
| AAM40449 | Human polypeptide | 64 . . . 482 | 283/432 | e−162 |
| | SEQ ID NO 5380— | | (65%) | |
| | Homo sapiens, | 128 . . . 558 | 356/432 | |
| | 680 aa. | | (81%) | |
| | [WO200153312-A1, | | | |
| | Jul. 26, 2001] | | | |

In a BLAST search of public sequence databases, the NOV15a protein was found to have homology to the proteins shown in the BLASTP data in Table 15E.

TABLE 15E
Public BLASTP Results for NOV15a

| | | NOV15a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9BQH3 | HYPOTHETICAL | 1 . . . 503 | 497/505 (98%) | 0.0 |
| | 55.7 KDA | 1 . . . 505 | 499/505 (98%) | |
| | PROTEIN—Homo | | | |
| | sapiens (Human), | | | |
| | 505 aa. | | | |
| P97756 | CA2+/ | 1 . . . 503 | 465/505 (92%) | 0.0 |
| | CALMODULIN- | 1 . . . 505 | 478/505 (94%) | |
| | DEPENDENT | | | |
| | PROTEIN KINASE | | | |
| | IV KINASE | | | |
| | ISOFORM—Rattus | | | |
| | norvegicus (Rat), | | | |
| | 505 aa. | | | |
| AAH17529 | SIMILAR TO | 1 . . . 503 | 464/505 (91%) | 0.0 |
| | CALCIUM/ | 1 . . . 505 | 478/505 (93%) | |
| | CALMODULIN- | | | |
| | DEPENDENT | | | |
| | PROTEIN KINASE | | | |
| | KINASE 1, | | | |
| | ALPHA—Mus | | | |
| | musculus (Mouse), | | | |
| | 505 aa. | | | |
| Q64572 | CA2+/ | 1 . . . 503 | 463/505 (91%) | 0.0 |
| | CALMODULIN- | 1 . . . 505 | 476/505 (93%) | |
| | DEPENDENT | | | |
| | PROTEIN KINASE | | | |
| | KINASE (EC | | | |
| | 2.7.1.37)—Rattus | | | |
| | norvegicus (Rat), | | | |
| | 505 aa. | | | |
| Q9R054 | CALCIUM/ | 1 . . . 503 | 454/505 (89%) | 0.0 |
| | CALMODULIN | 1 . . . 505 | 471/505 (92%) | |
| | DEPENDENT | | | |
| | PROTEIN KINASE | | | |
| | KINASE ALPHA— | | | |
| | Mus musculus | | | |
| | (Mouse), 505 aa. | | | |

PFam analysis predicts that the NOV15a protein contains the domains shown in the Table 15F.

TABLE 15F
Domain Analysis of NOV15a

| | NOV15a | Identities/Similarities | Expect |
|---|---|---|---|
| Pfam Domain | Match Region | for the Matched Region | Value |
| Pkinase: domain | 128 . . . 228 | 28/101 (28%) | 8.4e−16 |
| 1 of 2 | | 81/101 (80%) | |
| Pkinase: domain | 245 . . . 407 | 70/201 (35%) | 1.7e−52 |
| 1 of 2 | | 129/201 (64%) | |

### Example 16

The NOV16 clone was analyzed, and the nucleotide and predicted polypeptide sequences are shown in Table 16A.

TABLE 16A
NOV16 Sequence Analysis

| | SEQ ID NO:59 | 688 bp | |
|---|---|---|---|
| NOV16a | GACGCGGACCCGCCATGGCGCGGAAGAAGGTGCGTCCGCGGCTGATCGCGGAGCTGGC | | |
| CG57709-01 DNA Sequence | | | |
| | CCGCCGCGTGCGCGCCCTGCGGGAGCAACTGAACAGGCCGCGCGACTCCCAGCTCTAC | | |
| | GCGGTGGACTACGAGACCTTGACGCGGCCGTTCTCTGGACGCCGGCTGCCGGTCCGGG | | |
| | CCTGGGCCGACGTGCGCCGCGAGAGCCGCCTCTTGCAGCTGCTCGGCCGCCTCCCGCT | | |
| | CTTCGGCCTGGGCCGCCTGGTCACGCGCAAGTCCTGGCTGTGGCAGCACGACGAGCCG | | |
| | TGCTACTGGCGCCTCACGCGGGTGCGGCCCGACTACACGGCGCAGAACTTGGACCACG | | |
| | GGAAGGCCTGGGGCATCCTGACCTTCAAAGGTAAGGCTCGGGAGAGCGCGCGGGAGAT | | |
| | CGAACACGTCATGTACCATGACTGGCGGCTGGTGCCCAAGCACGAGGAGGAGGCCTTC | | |
| | ACCGCGTTCACGCCGGCGCCGGAAGACAGCCTGGCCTCCGTGCCGTACCCGCCTCTCC | | |
| | TCCGGGCCATGATTATCGCAGAACGACAGAAAAATGGAGACACAAGCACCGAGGAGCC | | |
| | CATGCTGAATGTGCAGAGGATACGCATGGAACCCTGGGATTACCCTGCAAAACAGGAA | | |
| | GACAAAGGAAGGGCCAAGGGCACCCCCGTCTAGAATGCCAGAACCAGCGG | | |
| | ORF Start: ATG at 15 | | ORF Stop: TAG at 669 |
| | SEQ ID NO:60 | 218 aa | MW at 25647.2 kD |

| | |
|---|---|
| NOV16a, | MARKKVRPRLIAELARRVRALREQLNRPRDSQLYAVDYETLTRPFSGRRLPVRAWADV |
| CG57709-01 Protein Sequence | |
| | RRESRLLQLLGRLPLFGLGRLVTRKSWLWQHDEPCYWRLTRVRPDYTAQNLDHGKAWG |
| | ILTFKGKARESAREIEHVMYHDWRLVPKHEEEAFTAFTPAPEDSLASVPYPPLLRAMI |
| | IAEROKNGDTSTEEPMLNVORIRMEPWDYPAKOEDKGRAKGTPV |

Further analysis of the NOV16a protein yielded the following properties shown in Table 16B.

TABLE 16B
Protein Sequence Properties NOV16a

| | |
|---|---|
| PSort | 0.9081 probability located in mitochondrial matrix space; |
| analysis: | 0.6000 probability located in mitochondrial inner membrane; |
| | 0.6000 probability located in mitochondrial intermembrane |
| | space; 0.6000 probability located in mitochondrial outer |
| | membrane |
| SignalP | No Known Signal Sequence Predicted |
| analysis: | |

A search of the NOV16a protein against the Geneseq database, a proprietary database that contains sequences published in patents and patent publication, yielded several homologous proteins shown in Table 16C.

TABLE 16C
Geneseq Results for NOV16a

| | | NOV16a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG81356 | Human AFP | 1 . . . 218 | 212/218 | e−125 |
| | protein sequence | | (97%) | |
| | SEQ ID NO: | 1 . . . 218 | 212/218 | |
| | 230—Homo | | (97%) | |
| | sapiens, 18 aa. | | | |
| | [WO200129221- | | | |
| | A2, Apr. 26, | | | |
| | 2001] | | | |
| AAU30525 | Novel human | 135 . . . 218 | 84/84 | 3e−45 |
| | secreted protein | | (100%) | |
| | [WO200179449- | 1 . . . 84 | 84/84 | |
| | A2, Oct. 25, | | (100%) | |
| | 2001] | | | |
| AAU30526 | Novel human | 187 . . . 217 | 31/31 | 4e−12 |
| | secreted protein | | (100%) | |
| | #1017—Homo | 12 . . . 42 | 31/31 | |
| | sapiens, 62 aa. | | (100%) | |
| | [WO200179449- | | | |
| | A2, Oct. 25, | | | |
| | 2001] | | | |

In a BLAST search of public sequence databases, the NOV16a protein was found to have homology to the proteins shown in the BLASTP data in Table 16D.

TABLE 16D
Public BLASTP Results for NOV16a

| | | NOV16a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9BV17 | HYPOTHETICAL | 1 . . . 218 | 214/218 (98%) | e−125 |
| | 25.7 KDA | 1 . . . 218 | 214/218 (98%) | |
| | PROTEIN—Homo | | | |
| | sapiens (Human), | | | |
| | 218 aa. | | | |
| P82930 | MITO- | 1 . . . 218 | 213/218 (97%) | e−124 |
| | CHONDRIAL 28S | 1 . . . 218 | 213/218 (97%) | |
| | RIBOSOMAL | | | |
| | PROTEIN S34 | | | |
| | (MRP-S34)—Homo | | | |
| | sapiens (Human), | | | |
| | 218 aa. | | | |
| CAC38606 | SEQUENCE 229 | 1 . . . 218 | 212/218 (97%) | e−124 |
| | FROM PATENT | 1 . . . 218 | 212/218 (97%) | |
| | WO0129221— | | | |
| | Homo sapiens | | | |
| | (Human), 218 aa. | | | |
| Q9JIK9 | TCE2 | 1 . . . 218 | 194/218 (88%) | e−114 |
| | (0610007F04RIK | 1 . . . 218 | 205/218 (93%) | |
| | PROTEIN)— | | | |
| | Mus musculus | | | |
| | (Mouse), 218 aa. | | | |
| Q9D957 | 0610007F04R1K | 1 . . . 218 | 193/218 (88%) | e−114 |
| | PROTEN— | 1 . . . 218 | 205/218 (93%) | |
| | Mus musculus | | | |
| | (Mouse), 218 aa. | | | |

PFam analysis predicts that the NOV16a protein contains the domains shown in the Table 16E.

TABLE 16E
Domain Analysis of NOV16a

| | | | |
|---|---|---|---|
| | NOV16a | Identities/Similarities | Expect |
| Pfam Domain | Match Region | for the Matched Region | Value |

No Significant Matches Found

### Example 17

The NOV17 clone was analyzed, and the nucleotide and predicted polypeptide sequences are shown in Table 17A.

TABLE 17A
NOV17 Sequence Analysis

| | SEQ ID NO:61 | 894 bp | |
|---|---|---|---|
| NOV17a, | CTCCGTGACCATGAAGGTCAAGGTCATCCCCGTGCTCGAGGACAACTACATGTACCTG | | |
| CG57700-01 DNA Sequence | | | |
| | GTCATCGAGGAGCTCACGCGCGAGGCGGTGGCCGTGGACGTGGCTGTGCCCAAGAGGC | | |
| | TGCTGGAGATCGTGGGCCGGGAGGGGGTGTCTCTGACCGCTGTGCTGACCACCCACCA | | |
| | TCACTGGGACCACGCGCGGGGAAACCCGGAGCTGGCGCGGCTTCGTCCCGGGCTGGCG | | |
| | GTGCTGGGCGCGGACGAGCGCATCTTCTCGCTGACGCGCAGGCTGGCGCACGGCGAGG | | |
| | AGCTGCAGTTCGGGGCCATCCACGTGCGTTGCCTCCTGACGCCCGGCCACACCGCCCG | | |
| | CCACATGAGCTACTTCCTGTGGGAGGACGATTGCCCGGACCCACCCGCCCTGTTCTCG | | |
| | GGTGGCGACGCGCTGTCGGTGGCCGGCTGCGGCTCGTGCCTGGAGGGCAGCGCCCAGC | | |
| | AGATGTACCAGAGCCTGGCCGAGCTGGGTACCCTGCCCCCCGAGACGAAGGTGTTCTG | | |
| | CGGCCACGAGCACACGCTTAGCAACCTGGAGTTTGCCCAGAAAGTGGAGCCCTGCAAC | | |
| | GACCACGTGAGAGCCAAGCTGTCCTGGGCTCAGAAGAGGGATGAGGATGACGTGCCCA | | |
| | CTGTGCCGTCGACTCTGGGCGAGGAGCGCCTCTACAACCCCTTCCTGCGGGTGGCGGA | | |
| | GGAGCCGGTGCGCAAGTTCACGGGCAAGGCGGTCCCCGCCGACGTCCTGGAGGCGCTA | | |
| | TGCAAGGAGCGGGCGCGCTTCGAACAGGCGGGCGAGCCGCGGCAGCCACAGGCGCGGG | | |
| | CCCTCCTTGCGCTGCAGTGGGGGCTCCTGAGTGCAGCCCCACACGACTGAGCCACCCA | | |
| | GACCCTCACAGGGCTGGGGCCTGC | | |
| | ORF Start: ATG at 11 | | ORF Stop: TGA at 860 |
| | SEQ ID NO:62 | 283 aa | MW at 31262.3 kD |
| NOV17a, | MKVKVIPVLEDNYMYLVIEELTREAVAVDVAVPKRLLEIVGREGVSLTAVLTTHHHWD | | |
| CG57700-01 Protein Sequence | | | |
| | HARGNPELARLRPGLAVLGADERIFSLTRRLAHGEELQFGAIHVRCLLTPGHTAGHMS | | |
| | YFLWEDDCPDPPALFSGGDALSVAGCGSCLEGSAQQMYQSLAELGTLPPETKVFCGHE | | |
| | HTLSNLEFAQKVEPCNDHVRAKLSWAQKRDEDDVPTVPSTLGEERLYNPFLRVAEEPV | | |
| | RKFTGKAVPADVLEALCKERARFEQAGEPRQPQARALLALQWGLLSAAPHD | | |
| | SEQ ID NO:63 | 888 bp | |
| NOV17b, | CTCCGTGACCATGAAGGTCAAGGTCATCCCCGTGCTCGAGGACAACTACATGTACCTG | | |
| CG57700-02 DNA Sequence | | | |
| | GTCATCGAGGAGCTCACGCGCGAGGCGGTGGCCGTGGACGTGGCTGTGCCCAAGAGGC | | |
| | TGCTGGAGATCGTGGGCCGGGAGGGGGTGTCTCTGACCGCTGTGCTGACCACCCACCA | | |
| | TCACTGGGACCACGCGCGGGGAAACCCGGAGCTGGCGCGGCTTCGTCCCGGGCTGGCG | | |
| | GTGCTGGGCGCGGACGAGCGCATCTTCTCGCTGACGCGCAGGCTGGCGCACGGCGAGG | | |
| | AGCTGCAGTTCGGGGCCATCCACGTGCGTTGCCTCCTGACGCCCGGCCACACCGCCGG | | |
| | CCACATGAGCTACTTCCTGTGGGAGGACGATTGCCCGGACCCACCCGCCCTGTTCTCG | | |
| | GGCGACGCGCTGTCGGTGGCCGGCTGCGGCTCGTGCCTGGAGGGCAGCGCCCAGCAGA | | |
| | TGTACCAGAGCCTGGCCGAGCTGGGTACCCTGCCCCCCGAGACGAAGGTGTTCTGCGG | | |
| | CCACGAGCACACACTTAGCAACCTGGAGTTTGCCCAGAAAGTGGAGCCCTGCAACGAC | | |
| | CACGTGAGAGCCAAGCTGTCCTGGGCTAAGAAGAGGGATGAGGATGACGTGCCCACTG | | |
| | TGCCGTCGACTCTGGGCGAGGAGCGCCTCTACAACCCCTTCCTGCGGGTGGCAGAGGA | | |
| | GCCGGTGCGCAAGTTCACGGGCAAGGCGGTCCCCGCCGACGTCCTGGAGGCGCTATGC | | |
| | AAGGAGCGGGCGCGCTTCGAACAGGCGGGCGAGCCGCGGCAGCCACAGGCGCGGGCCC | | |
| | TCCTTGCGCTGCAGTGGGGGCTCCTGAGTGCAGCCCCACACGACTGAGCCACCCAGAC | | |
| | CCTCACAGGGCTGGGCCT | | |
| | ORF Start: ATG at 11 | | ORF Stop: TGA at 857 |
| | SEQ ID NO:64 | 282 aa | MW at 31205.3 kD |
| NOV17b, | MKVKVIPVLEDNYMYLVIEELTREAVAVDVAVPKRLLEIVGREGVSLTAVLTTHHHWD | | |
| CG57700-02 Protein Sequence | | | |
| | HARGNPELARLRPGLAVLGADERIFSLTRRLAHGEELQFGAIHVRCLLTPGHTAGHMS | | |
| | YFLWEDDCPDPPALFSGDALSVAGCGSCLEGSAQQMYQSLAELGTLPPETKVFCGHEH | | |
| | TLSNLEFAQKVEPCNDHVRAKLSWAKKRDEDDVPTVPSTLGEERLYNPFLRVAEEPVR | | |
| | KFTGKAVPADVLEALCKERARFEQAGEPRQPQARALLALQWGLLSAAPHD | | |
| | SEQ ID NO:65 | 882 bp | |
| NOV17c, | ACCATGAAGGTCAAGGTCATCCCCGTGCTCGAGGACAACTACATGTACCTGGTCATCG | | |
| CG57700-03 DNA Sequence | | | |
| | AGGAGCTCACGCGCGAGGCGGTGGCCGTGGACGTGGCTGTGCCCAAGAGGCTGCTGGA | | |
| | GATCGTGGGCCGGGAGGGGGTGTCTCTGACCGCTGTGCTGACCACCCACCATCACTGG | | |
| | GACCACGCGCGGGGAAACCCGGAGCTGGCGCGGCTTCGTCCCGGGCTGGCGGTGCTGG | | |
| | GCGCGGACGAGCGCATCTTCTCGCTGACGCGCAGGCTGGCGCACGGCGAGGAGCTGCG | | |
| | GTTCGGGGCCATCCACGTGCGTTGCCTCCTGACGCCCGGCCACACCGCCGGCCACATG | | |
| | AGCTACTTCCTGTGGGAGGACGATTGCCCGGACCCACCCGCCCTGTTCTCGGGCGACG | | |
| | CGCTGTCGGTGGCCGGCTGCGGCTGGTCCGTGGAGGGCAGCCCCCAGGACATCTACCA | | |
| | GAGCCTGGCCGAGCTGGGTACCCTGCCCCCCGAGACGAAGGTGTTCTGCGGCCACGAG | | |
| | CACACGCTTAGCAACCTGGAGTTTGCCCAGAAAGTGGAGCCCTGCAACGACCACGTGA | | |
| | GAGCCAAGCTGTCCTGGGCTAAGAAGAGGGATGAGGATGACGTGCCCACTGTGCCGTC | | |
| | GACTCTGGGCGAGGAGCGCCTCTACAACCCCTTCCTGCGGGTGGCAGAGGAGCCGGTG | | |
| | CGCAAGTTCACGGGCAAGGCGGTCCCCGCCGACGTCCTGGAGGCGCTATGCAAGGAGC | | |
| | GGGCGCGCTCCGAACAGGCGGGCGAGCCGCGGCAGCCACAGGCGCGGGCCCTCCTTGC | | |
| | GCTGCAGTGGGGGCTCCTGAGTGCAGCCCCACACGACTGAGCCACCCAGACCCTCACA | | |
| | GGGCTGGGGCCT | | |
| | ORF Start: ATG at 4 | | ORF Stop: TGA at 850 |
| | SEQ ID NO:66 | 282 aa | MW at 31173.2 kD |
| NOV17c, | MKVKVIPVLEDNYMYLVIEELTREAVAVDVAVPKRLLEIVGREGVSLTAVLTTHHHWD | | |
| CG57700-03 Protein Sequence | | | |
| | HARGNPELARLRPGLAVLGADERIFSLTRRLAHGEELRFGAIHVRCLLTPGHTAGHMS | | |
| | YFLWEDDCPDPPALFSGDALSVAGCGSCLEGSAQQMYQSLAELGTLPPETKVFCGHEH | | |
| | TLSNLEFAQKVEPCNDHVRAKLSWAKKRDEDDVPTVPSTLGEERLYNPFLRVAEEPVR | | |
| | KFTGKAVPADVLEALCKERARSEQAGEPRQPQARALLALQWGLLSAAPHD | | |
| | SEQ ID NO:67 | 855 bp | |
| NOV17d, | ACCATGAAGGTCAAGGTCATCCCCGTGCTCGAGGACAACTACATGTACCTGGTCATCG | | |
| CG57700-04 DNA Sequence | | | |
| | AGGAGCTCACGCGCGAGGCGGTGGCCGTGGACGTGGCTGTGCCCAAGAGGCTGCTGGA | | |
| | GATCGTGGGCCGGGAGGGGGTGTCTCTGACCGCTGTGCTGACCACCCACTATCACTGG | | |
| | GACCACGCGCGGGGAAACCCGGAGCTGGCGCGGCTTCGTCCCGGGCTGGCGGTGCTGG | | |
| | GCGCGGACGAGCGCATCTTCTCGCTGACGCGCAGGCTGGCGCACGGCGAGGAGCTGCG | | |
| | GTTCGGGGCCATCCACGTGCGTTGCCTCCTGACGCCCGGCCACACCGCCGGCCACATG | | |
| | AGCTACTTCCTGTGGGAGGACGATTGCCCGGACCCACCCGCCCTGTTCTCGGGCGACG | | |
| | CGCTGTCGGTGGCCGGCTGCGGCTCGTGCCTGGAGGGCAGCGCCCAGCAGATGTACCA | | |
| | GAGCCTGGCCGAGCTGGGTACCCTGCCCCCCGAGACGAAGGTGTTCTGCGGCCACGAG | | |
| | CACACGCTTAGCAACCTGGAGTTTGCCCAGAAAGTGGAGCCCTGCAACGACCACAAGA | | |
| | GGGATGAGGATGACGTGCCCACTGTGCCGTCGACTCTGGGCGAGGAGCGCCTCTACAA | | |
| | CCCCTTCCTGCGGGTGGCAGAGGAGCCGGTGCGCAAGTTCACGGGCAAGGCGGTCCCC | | |
| | GCCGACGTCCTGGAGGCGCTATGCAAGGAGCGGGCGCGCTTCGAACAGGCGGGCGAGC | | |
| | CGCGGCAGCCACAGGCGCGGGCCCTCCTTGCGCTGCAGTGGGGGCTCCTGAGTGCAGC | | |
| | CCCACACGACTGAGCCACCCAGACCCTCACAGGGCTGGGGCCT | | |
| | ORF Start: ATG at 4 | | ORF Stop: TGA at 823 |
| | SEQ ID NO:68 | 273 aa | MW at 30219.1 kD |

| | |
|---|---|
| NOV17d, | MKVKVIPVLEDNYMYLVIEELTREAVAVDVAVPKRLLEIVGREGVSLTAVLTTHYHWD |
| CG57700-04 Protein Sequence | |
| | HARGNPELARLRPGLAVLGADERIFSLTRRLAHGEELRFGAIHVRCLLTPGHTAGHMS |
| | YFLWEDDCPDPPALFSGDALSVAGCGSCLEGSAQQMYQSLAELGTLPPETKVFCGHEH |
| | TLSNLEFAQKVEPCNDHKRDEDDVPTVPSTLGEERLYNPFLRVAEEPVRKFTGKAVPA |
| | DVLEALCKERARFEQAGEPRQPQARALLALQWGLLSAAPHD |

Sequence comparison of the above protein sequences yields the following sequence relationships shown in Table 17B.

TABLE 17B
Comparison of NOV17a against NOV17b through NOV17d.

| Protein | NOV17a Residues/ | Identities/Similarities |
|---|---|---|
| Sequence | Match Residues | for the Matched Region |
| NOV17b | 1 . . . 283 | 281/283 (99%) |
| | 1 . . . 282 | 282/283 (99%) |
| NOV17c | 1 . . . 283 | 279/283 (98%) |
| | 1 . . . 282 | 281/283 (98%) |
| NOV17d | 1 . . . 283 | 271/283 (95%) |
| | 1 . . . 273 | 273/283 (95%) |

Further analysis of the NOV17a protein yielded the following properties shown in Table 17C.

TABLE 17C
Protein Sequence Properties NOV17a

| | |
|---|---|
| PSort | 0.4500 probability located in cytoplasm; 0.3000 probability |
| analysis: | located in microbody (peroxisome); 0.1682 probability located |
| | in lysosome (lumen); 0.1000 probability located in |
| | mitochondrial matrix space |
| SignalP | No Known Signal Sequence Predicted |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAW80783 | Human bisphos- | 1 . . . 256 | 128/257 (49%) | 6e−72 |
| | phonate binding | 1 . . . 256 | 184/257 (70%) | |
| | protein, DP1 | | | |
| | (hDP1)—Homo | | | |
| | sapiens, 260 aa. | | | |
| | [WO9836064-A1, | | | |
| | Aug. 20, 1998] | | | |
| AAG10987 | Arabidopsis | 1 . . . 245 | 107/248 (43%) | 5e−53 |
| | thaliana protein | 1 . . . 246 | 160/248 (64%) | |
| | fragment SEQ ID | | | |
| | NO: 9531— | | | |
| | Arabidopsis | | | |
| | thaliana, 258 aa. | | | |
| | [EP1033405-A2, | | | |
| | Sep. 6, 2000] | | | |
| AAG10986 | Arabidopsis | 1 . . . 245 | 107/248 (43%) | 5e−53 |
| | thaliana protein | 11 . . . 256 | 160/248 (64%) | |
| | fragment SEQ ID | | | |
| | NO: 9530— | | | |
| | Arabidopsis | | | |
| | thaliana, 268 aa. | | | |
| | [EP1033405-A2, | | | |
| | Sep. 6, 2000] | | | |
| AAM78721 | Human protein | 1 . . . 226 | 100/227 (44%) | 6e−45 |
| | SEQ ID NO | 119 . . . 344 | 135/227 (59%) | |
| | 1383—Homo | | | |
| | sapiens, 385 aa. | | | |
| | [WO200157190- | | | |
| | A2, Aug. 9, | | | |
| | 2001] | | | |
| AAY71110 | Human Hydrolase | 1 . . . 226 | 100/227 (44%) | 6e−45 |
| | protein-8 | 95 . . . 320 | 135/227 (59%) | |
| | (HYDRL-8)— | | | |
| | Homo sapiens, | | | |
| | 361 aa. | | | |
| | [WO200028045- | | | |
| | A2, May 18, | | | |
| | 2000] | | | |

In a BLAST search of public sequence databases, the NOV17a protein was found to have homology to the proteins shown in the BLASTP data in Table 17E.

TABLE 17E
Public BLASTP Results for NOV17a

| | | NOV17a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9BT45 | SIMILAR TO RIKEN | 1 . . . 283 | 280/283 (98%) | e−163 |
| | CDNA 1500017E18 | 1 . . . 282 | 282/283 (98%) | |
| | GENE—Homo sapiens | | | |
| | (Human), 282 aa. | | | |
| Q9DB32 | 1500017E18R1K | 1 . . . 278 | 231/279 (82%) | e−133 |
| | PROTEIN—Mus | 1 . . . 278 | 251/279 (89%) | |
| | musculus (Mouse), | | | |
| | 283 aa. | | | |
| Q96S11 | SIMILAR TO | 1 . . . 128 | 217/228 (95%) | e−123 |
| | HAGH—Homo | 1 . . . 218 | 218/228 (95%) | |
| | sapiens (Human), | | | |
| | 218 aa. | | | |
| Q96NR5 | CDNA FLJ30279 | 1 . . . 133 | 132/133 (99%) | 3e−73 |
| | FIS, CLONE | 1 . . . 133 | 133/133 (99%) | |
| | BRACE2002772, | | | |
| | MODERATELY | | | |
| | SIMILAR TO | | | |
| | HYDROXYACYL- | | | |
| | GLUTATHIONE | | | |
| | HYDROLASE | | | |
| | (BC 3.1.2.6)—Homo | | | |
| | sapiens (Human), | | | |
| | 202 aa. | | | |
| O35952 | Hydroxyacyl- | 1 . . . 256 | 128/257 (49%) | 1e−71 |
| | glutathione hydrolase | 1 . . . 256 | 184/257 (70%) | |
| | (EC 3.1.2.6) | | | |
| | (Glyoxalase II) (Glx | | | |
| | II) (Round spermatid | | | |
| | protein RSP29)— | | | |
| | Rattus norvegicus | | | |
| | (Rat), 260 aa. | | | |

PFam analysis predicts that the NOV17a protein contains the domains shown in the Table 17F.

TABLE 17F
Domain Analysis of NOV17a

| | NOV17a | Identities/Similarities | Expect |
|---|---|---|---|
| Pfam Domain | Match Region | for the Matched Region | Value |
| lactamase_B: | 7 . . . 173 | 55/221 (25%) | 5.8e−32 |
| domain 1 of 1 | | 129/221 (58%) | |

### Example 18

The NOV18 clone was analyzed, and the nucleotide and predicted polypeptide sequences are shown in Table 18A.

TABLE 18A
NOV18 Sequence Analysis

| | SEQ ID NO:69 | 2109 bp | |
|---|---|---|---|
| NOV18a, | GGGTCCGGCGGGCATCGGCAAGACCATGGCGGCCAAAAATATCCTGTACGACTGGGCG | | |
| CG58553-01 DNA Sequence | | | |
| | GCGGGCAAGCTGTACCAGGGCCAGGTGGACTTCGCCTTCTTCATGCCCTGCGGCGAGC | | |
| | TGCTGGAGAGGCCGGGCACGCGCAGCCTGGCTGACCTGATCCTGGACCAGTGCCCCGA | | |
| | CCGCGGCGCGCCGGTGCCGCAGATGCTGGCCCAGCCGCAGCGGCTGCTCTTCATCCTG | | |
| | GACGGCGCGGACGAGCTGCCGGCGCTGGGGGGCCCCGAGGCCGCGCCCTGCACAGACC | | |
| | CCTTCGAGGCGGCGAGCGGCGCGCGGGTGCTAGGCGGGCTGCTGAGTAAGGCGCTGCT | | |
| | GCCCACGGCCCTCCTGCTGGTGACCACGCGCGCCGCCGCCCCCGGGAGGCTGCAGGGC | | |
| | CGCCTGTGTTCCCCGCAGTGCGCCGAGGTGCGCGGCTTCTCCGACAAGGACAAGAAGA | | |
| | AGTATTTCTACAAGTTCTTCCGGGATGAGAGGAGGGCCGAGCGCGCCTACCGCTTCGT | | |
| | GAAGGAGAACGAGACGCTGTTCGCGCTGTGCTTCGTGCCCTTCGTGTGCTGGATCGTG | | |
| | TGCACCGTGCTGCGCCAGCAGCTGGAGCTCGGTCGGGACCTGTCGCGCACGTCCAAGA | | |
| | CCACCACGTCAGTGTACCTGCTTTTCATCACCAGCGTTCTGAGCTCGGCTCCGGTAGC | | |
| | CGACGGGCCCCGGTTGCAGGGCGACCTGCGCAATCTGTGCCGCCTGGCCCGCGAGGGC | | |
| | GTCCTCGGACGCAGGGCGCAGTTTGCCGAGAAGGAACTGGAGCAACTGGAGCTTCGTG | | |
| | GCTCCAAAGTGCAGACGCTGTTTCTCAGCAAAAAGGAGCTGCCGGGCGTGCTGGAGAC | | |
| | AGAGGTCACCTACCAGTTCATCGACCAGAGCTTCCAGGAGTCCTTCGCGGCACTGTCC | | |
| | TACCTGCTGGAGGACGGCGGGGTGCCCAGGACCGCGGCTGGCGGCGTTGGGACACTCC | | |
| | TGCGTGGGGACGCCCAGCCGCACAGCCACTTGGTGCTCACCACGCGCTTCCTCTTCGG | | |
| | ACTGCTGAGCGCGGAGCGGATGCGCGACATCGAGCGCCACTTCGGCTGCATGGTTTCA | | |
| | GAGCGTGTGAAGCAGGAGGCCCTGCGGTGGGTGCAGGGACAGGGACAGGGCTGCCCCG | | |
| | GAGTGGCACCAGAGGTGACCGAGGGGGCCAAAGGGCTCGAGGACACCGAAGAGCCAGA | | |
| | GGAGGAGGAGGAGGGAGAGGAGCCCAACTACCCACTGGAGTTGCTGTACTGCCTGTAC | | |
| | GAGACGCAGGAGGACGCGTTTGTGCGCCAAGCCCTGGGCCGGTTCCCGGAGCTGGCGC | | |
| | TGCAGCGAGTGCGCTTCTGCCGCATGGACGTGGCTGTTCTGAGCTACTGCGTGAGGTG | | |
| | CTGCCCTGCTGCACAGGCACTGCGGCTGATCAGCTGCAGATTGGTTGCTGCGCAGGAG | | |
| | AAGAAGAAGAAGAGCCTGGGGAAGCGGCTCCAGGCCAGCCTGGGCACCACAAAACAAC | | |
| | TGCCAGCCTCCCTTCTTCATCCACTCTTTCAGGCAATGACTGACCCACTGTGCCATCT | | |
| | GAGCAGCCTCACGCTGTCCCACTGCAAACTCCCTGACGCGGTCTGCCGAGACCTTTCT | | |
| | GAGGCCCTGAGGGCAGCCCCCGCACTGACGGAGCTGGGCCTCCTCCACAACAGGCTCA | | |
| | GTGAGGCAGGACTGCGTATGCTGAGTGAGGGCCTAGCCTGGCCGCAGTGCAGGGTGCA | | |
| | GACGGTCAGGGTACAGCTGCCTGACCCCCAGCGAGGGCTCCAGTACCTGGTGGGTATG | | |
| | CTTCGGCAGAGCCCTGCCCTGACCACCCTGGATCTCAGCGGCTGCCAACTGCCCGCCC | | |
| | CCATGGTGACCTACCTGTGTGCAGTCCTGCAGCACCAGGGATGCGGCCTGCAGACCCT | | |
| | CAGTCTGGCCTCTGTGGAGCTGAGCGAGCAGTCACTACAGGAGCTTCAGGCTGTGAAG | | |
| | AGAGCAAAGCCGGATCTGGTCATCACACACCCAGCGCTGGACGGCCACCCACAACCTC | | |
| | CCAAGGAACTCATCTCGACCTTCTGAGGCTCTGGTGGCCAGAGCAGGGTGGAAGACCC | | |
| | TAGTCAAAGTCCCTGTGGAGA | | |
| | ORF Start: ATG at 26 | | ORF Stop: TGA at 2054 |
| | SEQ ID NO:70 | 676 aa | MW at 74650.3 kD |

| | |
|---|---|
| NOV18a, | MAAKNILYDWAAGKLYQGQVDFAFFMPCGELLERPGTRSLADLILDQCPDRGAPVPQM |
| CG58553-01 Protein Sequence | |
| | LAQPQRLLFILDGADELPALGGPEAAPCTDPFEAASGARVLGGLLSKALLPTALLLVT |
| | TRAAAPGRLQGRLCSPQCAEVRGFSDKDKKKYFYKFFRDERRAERAYRFVKENETLFA |
| | LCFVPFVCWIVCTVLRQQLELGRDLSRTSKTTTSVYLLFITSVLSSAPVADGPRLQGD |
| | LRNLCRLAREGVLGRRAQFAEKELEQLELRGSKVQTLFLSKKELPGVLETEVTYQFID |
| | QSFQESFAALSYLLEDGGVPRTAAGGVGTLLRGDAQPHSHLVLTTRFLFGLLSAERMR |
| | DIERHFGCMVSERVKQEALRWVQGQGQGCPGVAPEVTEGAKGLEDTEEPEEEEEGEEP |
| | NYPLELLYCLYETQEDAFVRQALGRFPELALQRVRFCRMDVAVLSYCVRCCPAAQALR |
| | LISCRLVAAQEKKKKSLGKRLQASLGTTKQLPASLLHPLFQAMTDPLCHLSSLTLSHC |
| | KLPDAVCRDLSEALRAAPALTELGLLHNRLSEAGLRMLSEGLAWPQCRVQTVRVQLPD |
| | PQRGLQYLVGMLRQSPALTTLDLSGCQLPAPMVTYLCAVLQHQGCGLQTLSLASVELS |
| | EQSLQELQAVKRAKPDLVITHPALDGHPQPPKELISTF |

Further analysis of the NOV18a protein yielded the following properties shown in Table 18B.

TABLE 18B
Protein Sequence Properties NOV18a

| | |
|---|---|
| Psort | 0.7400 probability located in nucleus; 0.6000 probability |
| analysis: | located in endoplasmic reticulum (membrane); 0.3000 |
| | probability located in microbody (peroxisome); 0.1000 |
| | probability located in mitochondrial inner membrane |
| SignalP | No Known Signal Sequence Predicted |
| analysis: | |

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAE04546 | Human G-protein | 1 . . . 676 | 671/682 (98%) | 0.0 |
| | coupled receptor- | 210 . . . 891 | 671/682 (98%) | |
| | 2 (GCREC-2) | | | |
| | protein—Homo | | | |
| | sapiens, 891 aa. | | | |
| | [WO200142288- | | | |
| | A2, Jun. 14, | | | |
| | 2001] | | | |
| AAU00023 | Human activated | 1 . . . 633 | 605/695 (87%) | 0.0 |
| | T-lymphocyte | 210 . . . 904 | 610/695 (87%) | |
| | associated | | | |
| | sequence 2, | | | |
| | ATLAS-2— | | | |
| | Homo sapiens, | | | |
| | 1851 aa. | | | |
| | [WO200114564- | | | |
| | A2, Mar. 1, 2001] | | | |
| ABB11735 | Human vaso- | 1 . . . 490 | 485/490 (98%) | 0.0 |
| | pressin receptor | 106 . . . 595 | 485/490 (98%) | |
| | homologue, SEQ | | | |
| | ID NO:2105— | | | |
| | Homo sapiens, | | | |
| | 597 aa. | | | |
| | [WO200157188- | | | |
| | A2, Aug. 9, | | | |
| | 2001] | | | |
| AAR33389 | AII/AVPv2 | 193 . . . 670 | 322/481 (66%) | e−174 |
| | receptor— | 1 . . . 480 | 371/481 (76%) | |
| | Synthetic, 481 aa. | | | |
| | [WO9305073-A, | | | |
| | Mar. 18, 1993] | | | |
| AAM89960 | Human immune/ | 1 . . . 274 | 265/274 (96%) | e−151 |
| | haematopoietic | 9 . . . 282 | 266/274 (96%) | |
| | antigen SEQ ID | | | |
| | NO:17553— | | | |
| | Homo sapiens, | | | |
| | 329 aa. | | | |
| | [WO200157182- | | | |
| | A2, Aug. 9, | | | |
| | 2001] | | | |

In a BLAST search of public sequence databases, the NOV18a protein was found to have homology to the proteins shown in the BLASTP data in Table 18D.

TABLE 18D
Public BLASTP Results for NOV18a

| | | NOV18a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| CAC34689 | SEQUENCE 3 | 1 . . . 633 | 605/695 (87%) | 0.0 |
| | FROM PATENT | 210 . . . 904 | 610/695 (87%) | |
| | WO0114564— | | | |
| | Homo sapiens | | | |
| | (Human), | | | |
| | 1851 aa. | | | |
| Q91WS2 | HYPO- | 107 . . . 659 | 390/557 (70%) | 0.0 |
| | THETICAL | 1 . . . 554 | 450/557 (80%) | |
| | 62.5 KDA | | | |
| | PROTEIN— | | | |
| | Mus musculus | | | |
| | (Mouse), 556 aa | | | |
| | (fragment). | | | |
| Q63035 | VASOPRESSIN | 193 . . . 670 | 324/483 (67%) | e−173 |
| | RECEPTOR— | 1 . . . 482 | 372/483 (76%) | |
| | Rattus norvegicus | | | |
| | (Rat), 483 aa. | | | |
| AAL12498 | CRYOPYRIN— | 3 . . . 657 | 232/709 (32%) | 5e−94 |
| | Homo sapiens | 234 . . . 914 | 355/709 (49%) | |
| | (Human), 920 aa. | | | |
| AAL12497 | CRYOPYRIN— | 3 . . . 648 | 223/658 (33%) | 6e−93 |
| | Homo sapiens | 234 . . . 848 | 344/658 (51%) | |
| | (Human), 1034 | | | |
| | aa. | | | |

PFam analysis predicts that the NOV18a protein contains the domains shown in the Table 18E.

TABLE 18E
Domain Analysis of NOV18a

| | | | |
|---|---|---|---|
| | NOV18a | Identities/Similarities | Expect |
| Pfam Domain | Match Region | for the Matched Region | Value |

No Significant Matches Found

### Example 19

The NOV19 clone was analyzed, and the nucleotide and predicted polypeptide sequences are shown in Table 19A.

TABLE 19A
NOV19 Sequence Analysis

| | SEQ ID NO:71 | 2686 bp | |
|---|---|---|---|
| NOV19a, | CCGGCGGCGTCTCCACAGCATGAATTACCCGGGCCGCGGGTCCCCACGGAGCCCCGAG | | |
| CG58626-01 DNA Sequence | | | |
| | CATAACGGCCGAGGCGGCGGCGGCGGCGCCTGGGAGCTGGGCTCAGACGCGAGGCCAG | | |
| | CGTTCGGCGGCGGCGTCTGCTGCTTCGAGCACCTGCCCGGCGGGGACCCGGACGACGG | | |
| | CGACGTGCCCCTGGCCCTGCTGCGCGGGGAACCCGGGCTGCATTTGGCGCCGGGCACC | | |
| | GACGACCACAACCACCACCTCGCGCTGGACCCCTGCCTCAGTGACGAGAACTATGACT | | |
| | TCAGCTCCGCCGAGTCGGGCTCCTCGCTGCGCTACTACAGCGAGGGTGAGAGCGGCGG | | |
| | CGGCGGCAGCTCCTTGTCGCTGCACCCGCCGCAGCAGCCTCCGCTGGTCCCGACGAAC | | |
| | TCGGGGGGCGGCGGCGCGACAGGAGGGTCCCCCGGGGAAAGGAAACGTACCCGCCTTG | | |
| | GCGGCCCGGCGGCCCGGCACCGCTATGAGGTAGTGACGGAGCTGGGCCCGGAGGAGGT | | |
| | ACGCTGGTTCTACAAGGAGGACAAGAAGACCTGGAAGCCCTTCATCGGCTACGACTCG | | |
| | CTCCGCATCGAGCTCGCCTTCCGGACCCTGCTGCAGACCACGGGTGCCCGGCCCCAGG | | |
| | GCGGGGACCGGGACGGCGACCATGTGTGCTCCCCCACGGGCCCAGCCTCCAGTTCCGG | | |
| | AGAAGATGACGATGAGGACCGCGCCTGCGGCTTCTGCCAGAGTACGACGGGGCACGAG | | |
| | CCGGAGATGGTGGAGCTTGTGAACATCGAGCCTGTGTGCGTGCGGGGCGGCCTCTACG | | |
| | AGGTGGATGTGACCCAAGGAGAGTGCTACCCGGTGTACTGGAACCGTGCTGATAAAAT | | |
| | ACCAGTAATGCGTGGACAGTGGTTTATTGACGGCACTTGGCAGCCTCTAGAAGAGGAA | | |
| | GAAAGTAATTTAATTGAGCAAGAACATCTCAATTGTTTTAGGGGCCAGCAGATGCAGG | | |
| | AAAATTTCGATATTGAAGTGTCAAAATCCATAGATGGAAAAGATGCTGTTCATAGTTT | | |
| | CAAGTTGAGTCGAAACCATGTGGACTGGCACAGTGTGGATGAAGTATATCTTTATAGT | | |
| | GATGCAACAACATCTAAAATTGCAAGAACAGTTACCCAAAAACTGGGATTTTCTAAAG | | |
| | CATCAAGTAGTGGTACCAGACTTCATAGAGGTTATGTAGAAGAAGCCACATTAGAAGA | | |
| | CAAGCCATCACAGACTACCCATATTGTATTTGTTGTGCATGGCATTGGGCAGAAAATG | | |
| | GACCAAGGAAGAATTATCAAAAATACAGCTATGATGAGAGAAGCTGCAAGAAAAATAG | | |
| | AAGAAAGGCATTTTTCCAACCATGCAACACATGTTGAATTTCTGCCTGTTGAGTGGCG | | |
| | GTCAAAACTTACTCTTGATGGAGACACTGTTGATTCCATTACTCCTGACAAAGTACGA | | |
| | GGTTTAAGGGATATGCTGAACAGCAGTGCAATGGACATAATGTATTATACTAGTCCAC | | |
| | TTTATAGAGATGAACTAGTTAAAGGCCTTCAGCAAGAGCTGAATCGATTGTATTCCCT | | |
| | TTTCTGTTCTCGGAATCCAGACTTTGAAGAAAAAGGGGGTAAAGTCTCAATAGTATCA | | |
| | CATTCCTTGGGATGTGTAATTACTTATGACATAATGACTGGCTGGAATCCAGTTCGGC | | |
| | TGTATGAACAGTTGCTGCAAAAGGAAGAAGAGTTGCCTGATGAACGATGGATGAGCTA | | |
| | TGAAGAACGACATCTTCTTGATGAACTCTATATAACTAAACGACGGCTGAAGGAAATA | | |
| | GAAGAACGGCTTCACGGATTGAAAGCATCATCTATGACACAAACACCTGCCTTAAAAT | | |
| | TTAAGGTAGAGAATTTCTTCTGTATGGGATCCCCATTAGCAGTTTTCTTGGCGTTGCG | | |
| | TGGCATCCGCCCAGGAAATACTGGAAGTCAAGACCATATTTTGCCTAGAGAGATTTGT | | |
| | AACCGGTTACTAAATATTTTTCATCCTACAGATCCAGTGGCTTATAGATTAGAACCAT | | |
| | TAATACTGAAACACTACAGCAACATTTCACCTGTCCAGATCCACTGGTACAATACTTC | | |
| | AAATCCTTTACCTTATGAACATATGAAGCCAAGCTTTCTCAACCCAGCTAAAGAACCT | | |
| | ACCTCAGTTTCAGAGAATGAAGGCATTTCAACCATACCAAGCCCTGTGACCTCACCAG | | |
| | TTTTGTCCCGCCGACACTATGGAGAATCTATAACAAATATAGGCAAAGCAAGCATATT | | |
| | AGGTGCTGCTAGCATTGGAAAGGGACTTGGAGGAATGTTGTTCTCAAGATTTGGACGT | | |
| | TCATCTACAACACAGTCATCTGAAACATCAAAAGACTCAATGGAAGATGAGAAGAAGC | | |
| | CAGTTGCCTCACCTTCTGCTACCACCGTAGGGACACAGACCCTTCCACATAGCAGTTC | | |
| | TGGCTTCCTCGATTCTGCAGTGGAGTTGGATCACAGGATTGATTTTGAACTCAGAGAA | | |
| | GGCCTTGTGGAGAGCCGCTATTGGTCAGCTGTCACGTCGCATACTGCCTATTGGTCAT | | |
| | CCTTGGATGTTGCCCTTTTTCTTTTAACCTTCATGTATAAACATGAGCACGATGATGA | | |
| | TGCAAAACCCAATTTAGATCCAATCTGAACTCTCTTGAAGGACATGAATGGCCTAAAA | | |
| | CTGATTTTTTTTTTTTCC | | |
| | ORF Start: ATG at 20 | | ORF Stop: TGA at 2636 |
| | SEQ ID NO:72 | 872 aa | MW at 97063.4 kD |

| | |
|---|---|
| NOV19a, | MNYPGRGSPRSPEHNGRGGGGGAWELGSDARPAFGGGVCCFEHLPGGDPDDGDVPLAL |
| CG58626-01 Protein Sequence | |
| | LRGEPGLHLAPGTDDHNHHLALDPCLSDENYDFSSAESGSSLRYYSEGESGGGGSSLS |
| | LHPPQQPPLVPTNSGGGGATGGSPGERKRTRLGGPAARHRYEVVTELGPEEVRWFYKE |
| | DKKTWKPFIGYDSLRIELAFRTLLQTTGARPQGGDRDGDHVCSPTGPASSSGEDDDED |
| | RACGFCQSTTGHEPEMVELVNIEPVCVRGGLYEVDVTQGECYPVYWNRADKIPVMRGQ |
| | WFIDGTWQPLEEEESNLIEQEHLNCFRGQQMQENFDIEVSKSIDGKDAVHSFKLSRNH |
| | VDWHSVDEVYLYSDATTSKIARTVTQKLGFSKASSSGTRLHRGYVEEATLEDKPSQTT |
| | HIVFVVHGIGQKMDQGRIIKNTAMMREAARKIEERHFSNHATHVEFLPVEWRSKLTLD |
| | GDTVDSITPDKVRGLRDMLNSSAMDIMYYTSPLYRDELVKGLQQELNRLYSLFCSRNP |
| | DFEEKGGKVSIVSHSLGCVITYDIMTGWNPVRLYEQLLQKEEELPDERWMSYEERHLL |
| | DELYITKRRLKEIEERLHGLKASSMTQTPALKFKVENFFCMGSPLAVFLALRGIRPGN |
| | TGSQDHILPREICNRLLNIFHPTDPVAYRLEPLILKHYSNISPVQIHWYNTSNPLPYE |
| | HMKPSFLNPAKEPTSVSENEGISTIPSPVTSPVLSRRHYGESITNIGKASILGAASIG |
| | KGLGGMLFSRFGRSSTTQSSETSKDSMEDEKKPVASPSATTVGTQTLPHSSSGFLDSA |
| | VELDHRIDFELREGLVESRYWSAVTSHTAYWSSLDVALFLLTFMYKHEHDDDAKPNLD |
| | PI |

Further analysis of the NOV19a protein yielded the following properties shown in Table 19B.

TABLE 19B
Protein Sequence Properties NOV19a

| | |
|---|---|
| PSort | 0.4555 probability located in microbody (peroxisome); 0.4500 |
| analysis: | probability located in cytoplasm; 0.1000 probability located in |
| | mitochondrial matrix space; 0.1000 probability located in |
| | lysosome (lumen) |
| SignalP | No Known Signal Sequence Predicted |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| | | NOV19a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG64151 | Arabidopsis | 257 . . . 547 | 104/316 (32%) | 1e−38 |
| | thaliana | 156 . . . 454 | 156/316 (48%) | |
| | gravitropism | | | |
| | protein— | | | |
| | Arabidopsis | | | |
| | thaliana, 933 aa. | | | |
| | [JP2001120279- | | | |
| | A, May 8, 2001] | | | |
| AAM41595 | Human poly- | 261 . . . 548 | 94/301 (31%) | 6e−25 |
| | peptide SEQ ID | 52 . . . 328 | 138/301 (45%) | |
| | NO 6526−Homo | | | |
| | sapiens, 677 aa. | | | |
| | [WO200153312- | | | |
| | A1, Jul. 26, 2001] | | | |
| AAB92643 | Human protein | 119 . . . 608 | 132/524 (25%) | 2e−24 |
| | sequence SEQ ID | 226 . . . 664 | 204/524 (38%) | |
| | NO:10972— | | | |
| | Homo sapiens, | | | |
| | 1000 aa. | | | |
| | [EP1074617-A2, | | | |
| | Feb. 7, 2001] | | | |
| AAM39809 | Human poly- | 274 . . . 548 | 90/288 (31%) | 4e−23 |
| | peptide SEQ ID | 3 . . . 266 | 131/288 (45%) | |
| | NO 2954—Homo | | | |
| | sapiens, 615 aa. | | | |
| | [WO200153312- | | | |
| | A1, Jul. 26, 2001] | | | |
| AAB93825 | Human protein | 404 . . . 608 | 76/229 (33%) | 6e−23 |
| | sequence SEQ ID | 227 . . . 449 | 113/229 (49%) | |
| | NO:13636— | | | |
| | Homo sapiens, | | | |
| | 694 aa. | | | |
| | [EP1074617-A2, | | | |
| | Feb. 7, 2001] | | | |

In a BLAST search of public sequence databases, the NOV19a protein was found to have homology to the proteins shown in the BLASTP data in Table 19D.

TABLE 19D
Public BLASTP Results for NOV19a

| | | NOV19a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| O46606 | PHOSPHATIDIC | 1 . . . 872 | 802/876 (91%) | 0.0 |
| | ACID- | 1 . . . 875 | 829/876 (94%) | |
| | PREFERRING | | | |
| | PHOSPHO- | | | |
| | LIPASE A1— | | | |
| | Bos taurus | | | |
| | (Bovine), 875 aa. | | | |
| Q9C0F8 | KIAA1705 | 378 . . . 872 | 493/495 (99%) | 0.0 |
| | PROTEIN— | 4 . . . 498 | 494/495 (99%) | |
| | Homo sapiens | | | |
| | (Human), 498 aa | | | |
| | (fragment). | | | |
| Q96LL2 | CDNA FLJ25408 | 419 . . . 872 | 453/454 (99%) | 0.0 |
| | FIS, CLONE | 1 . . . 454 | 454/454 (99%) | |
| | TST02965, | | | |
| | HIGHLY | | | |
| | SIMILAR TO | | | |
| | BOS TAURUS | | | |
| | PHOSPHATIDIC | | | |
| | ACID- | | | |
| | PREFERRING | | | |
| | PHOSPHO- | | | |
| | LIPASE A1 | | | |
| | MRNA—Homo | | | |
| | sapiens (Human), | | | |
| | 454 aa. | | | |
| AAH18552 | HYPO- | 624 . . . 869 | 224/246 (91%) | e−130 |
| | THETICAL | 1 . . . 246 | 236/246 (95%) | |
| | 27.3 KDA | | | |
| | PROTEIN—Mus | | | |
| | musculus | | | |
| | (Mouse), 249 aa | | | |
| | (fragment). | | | |
| AAL32232 | HYPO- | 122 . . . 867 | 255/794 (32%) | 6e−91 |
| | THETICAL | 11 . . . 750 | 374/794 (46%) | |
| | 85.1 KDA | | | |
| | PROTEIN— | | | |
| | Caenorhabditis | | | |
| | elegans, 753 aa. | | | |

PFam analysis predicts that the NOV19a protein contains the domains shown in the Table 19E.

TABLE 19E
Domain Analysis of NOV19a

| | NOV19a | Identities/Similarities | Expect |
|---|---|---|---|
| Pfam Domain | Match Region | for the Matched Region | Value |
| DUF203: domain | 252 . . . 458 | 42/219 (19%) | 7.5 |
| 1 of 1 | | 105/219 (48%) | |
| DDHD: domain | 611 . . . 858 | 96/266 (36%) | 3.3e−116 |
| 1 of 1 | | 236/266 (89%) | |

### Example 20

The NOV20 clone was analyzed, and the nucleotide and predicted polypeptide sequences are shown in Table 20A.

TABLE 20A
NOV20 Sequence Analysis

| | SEQ ID NO:73 | 773 bp | |
|---|---|---|---|
| NOV20a, | GGTAAGGACACAAGATGCCAAATAGGGTAAGGAATGGTCCAGAAACCTGTGAACTCTG | | |
| CG57597-01 DNA Sequence | | | |
| | CATTGCAGGCATGCACCACCACTCCTGGCTAATTTTTTGTATTTTTAGTGCCATCGAA | | |
| | TCCGGCTCAAACCTTTTATTTCTCTTATGTAAAAGCTGTGTACTTCAGAAAAACATGT | | |
| | ACAGTTATCCCTGGCAGTGCCGGGGTGGGGTCTGCGCGGCCCTGGAGGCCTGGCCGGC | | |
| | CTTGCAGATCGCTGTGGAGAATGGCTTCGGGGGTGTGCACAGCCAGGAGAAGGCCAAG | | |
| | TGGCTGGGGGGTGCAGTGGAGGATTACTTCATGCGCAATGCTGACTTGGAGCTAGATG | | |
| | AGGTGGAAGACTTCCTTGGAGAGCTGTTGACCAACGAGTTTGATACAGTTGTGGAAGA | | |
| | CGGGAGTCTGCCCCAGGTGAGCCAGCAACTGCAGACCATGTTCCACCACTTCCAGAGG | | |
| | GGTGATGGGGCTGCTCTGAGGGAGATGGCCTCCTGCATCACTCAGAGAAAATGCAAGG | | |
| | TCACAGCCACTGCACTTAAGACAGCTAGAGAGACTGATGAGGATGAAGATGATGTGGA | | |
| | CAGTGTGGAAGAGATGGAGGTCACAGCTACGAATGATGGGGCTGCTACAGATGGGGTC | | |
| | TGCCCCCAGCCTGAACCCTCTGATCCAGACGCTCAGACTATTAAGGAAGAGGATATAG | | |
| | TGGAAGATGGCTGGACCATTGTCCGGAGAAAAAAATGAGTGGGGATGATTGGAAATGG | | |
| | CTTTGGGCCCTTATTTGCT | | |
| | ORF Start: ATG at 15 | | ORF Stop: TGA at 732 |
| | SEQ ID NO:74 | 239 aa | MW at 26579.5 kD |

| | |
|---|---|
| NOV20a, | MPNRVRNGPETCELCIAGMHHHSWLIFCIFSAIESGSNLLFLLCKSCVLQKNMYSYPW |
| CG57597-O1 Protein Sequence | |
| | QCRGGVCAALEAWPALQIAVENGFGGVHSQEKAKWLGGAVEDYFMRNADLELDEVEDF |
| | LGELLTNEFDTVVEDGSLPQVSQQLQTMFHHFQRGDGAALREMASCITQRKCKVTATA |
| | LKTARETDEDEDDVDSVEEMEVTATNDGAATDGVCPQPEPSDPDAQTIKEEDIVEDGW |
| | TIVRRKK |

Further analysis of the NOV20a protein yielded the following properties shown in Table 20B.

TABLE 20B
Protein Sequence Properties NOV20a

| | |
|---|---|
| PSort | 0.3000 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV20a protein against the Geneseq database, a proprietary database that contains sequences published in patents and patent publication, yielded several homologous proteins shown in Table 20C.

TABLE 20C
Geneseq Results for NOV20a

| | | NOV20a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG81374 | Human AFP | 61 . . . 239 | 178/179 (99%) | e−101 |
| | protein sequence | 13 . . . 191 | 178/179 (99%) | |
| | SEQ ID NO: | | | |
| | 266—Homo | | | |
| | sapiens, 191 aa. | | | |
| | [WO200129221- | | | |
| | A2, Apr. 26, | | | |
| | 2001] | | | |
| AAG57770 | Arabidopsis | 63 . . . 239 | 56/182 (30%) | 1e−13 |
| | thaliana protein | 18 . . . 178 | 94/182 (50%) | |
| | Arabidopsis | | | |
| | thaliana, 184 aa. | | | |
| | [EP1033405-A2, | | | |
| | Sep. 6, 2000] | | | |
| AAG57771 | Arabidopsis | 74 . . . 239 | 52/171 (30%) | 2e−11 |
| | thaliana protein | 1 . . . 150 | 89/171 (51%) | |
| | fragment SEQ ID | | | |
| | NO:74487— | | | |
| | Arabidopsis | | | |
| | thaliana, 156 aa. | | | |
| | [EP1033405-A2, | | | |
| | Sep. 6, 2000] | | | |

In a BLAST search of public sequence databases, the NOV20a protein was found to have homology to the proteins shown in the BLASTP data in Table 20D.

TABLE 20D
Public BLASTP Results for NOV20a

| | | NOV20a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q969E8 | UNKNOWN | 61 . . . 239 | 178/179 (99%) | e−101 |
| | (PROTEIN FOR | 13 . . . 191 | 178/179 (99%) | |
| | MGC:20451) | | | |
| | (PROTEIN FOR | | | |
| | IMAGE:3953868)— | | | |
| | Homo sapiens | | | |
| | (Human), 191 aa. | | | |
| Q9NAD8 | Y51H4A.15 | 1 . . . 239 | 66/239 (27%) | 5e−23 |
| | PROTEIN— | 1 . . . 225 | 122/239 (50%) | |
| | Caenorhabditis | | | |
| | elegans, 225 aa. | | | |
| Q06672 | HIGHLY ACIDIC | 63 . . . 238 | 46/177 (25%) | 5e−11 |
| | C-TERMINUS— | 79 . . . 244 | 82/177 (45%) | |
| | Saccharomyces | | | |
| | cerevisiae (Baker's | | | |
| | yeast), 249 aa. | | | |
| Q9VB10 | CG14543 | 71 . . . 238 | 49/174 (28%) | 2e−10 |
| | PROTEIN— | 24 . . . 195 | 81/174 (46%) | |
| | Drosophila | | | |
| | melanogaster | | | |
| | (Fruit fly), 195 aa. | | | |
| Q9UUA9 | HYPOTHETICAL | 70 . . . 239 | 42/172 (24%) | 2e−06 |
| | HIGHLY ACIDIC | 22 . . . 178 | 83/172 (47%) | |
| | C-TERMINUS | | | |
| | PROTEIN— | | | |
| | Schizosaccharo- | | | |
| | myces pombe | | | |
| | (Fission yeast), | | | |
| | 179 aa. | | | |

PFam analysis predicts that the NOV20a protein contains the domains shown in the Table 20E.

TABLE 20E
Domain Analysis of NOV20a

| | | | |
|---|---|---|---|
| | NOV20a | Identities/Similarities | Expect |
| Pfam Domain | Match Region | for the Matched Region | Value |

No Significant Matches Found

### Example 21

The NOV21 clone was analyzed, and the nucleotide and predicted polypeptide sequences are shown in Table 21A.

TABLE 21A
NOV21 Sequence Analysis

| | SEQ ID NO:75 | 7741 bp | |
|---|---|---|---|
| NOV21a, | TTGTCTCTTTGTGTTTTCCAGACATTCTAAGTGAGACTGTCCACATCATCTAGGAAAA | | |
| CG57804-01 DNA Sequence | | | |
| | TGGTGGCCCTGTCCTTAAAGATTTGTGTGCGCCACTGCAACGTGGTGAAGACCATGCA | | |
| | GTTTGAACCATCTACAGCTGTGTACGATGCGTGTCGAGTCATTCGGGAACGGGTGCCT | | |
| | GAGGCACAAACTGGGCAAGCTTCTGACTATGGACTCTTTCTTTCGGATGAAGACCCGA | | |
| | GGAAAGGGATTTGGCTGGAAGCGGGCAGAACACTGGATTACTACATGTTGCGGAATGG | | |
| | GGATATTTTGGAATATAAAAAGAAACAGAGACCTCAGAAAATCCGGATGCTGGATGGA | | |
| | TCTGTGAAGACAGTGATGGTGGATGATTCCAAGACTGTGGGGGAGCTCCTGGTCACTA | | |
| | TTTGTAGCAGAATAGGAATAACAAATTATGAAGAATACTCCTTAATCCAAGAAACTAT | | |
| | TGAAGAAAAGAAAGAGGAAGGAACGGGCACACTCAAAAAAGACAGGACACTGTTACGA | | |
| | GATGAGAGGAAAATGGAGAAGTTGAAGGCCAAGCTGCACACAGATGATGACCTAAATT | | |
| | GGCTGGATCACAGCCGAACATTCAGAGAACAAGGAGTAGATGAAAACGAAACGTTGCT | | |
| | GCTTAGACGGAAGTTCTTTTACTCTGATCAGAATGTAGATTCGAGAGACCCCGTGCAG | | |
| | CTGAACTTGCTTTATGTTCAGGCACGGGATGACATCCTGAATGGCTCTCACCCTGTCT | | |
| | CCTTCGAGAAACCTTGTGAGTTTGGTGGATTTCAAGCCCAGATACAATTTGGACCTCA | | |
| | TGTGGAACATAAACACAAACCTGGATTTTTAGATCTGAAGGAATTCCTGCCCAAAGAA | | |
| | TATATCAAGCAGAGAGGAGCTGAAAAGAGGATCTTTCAGGAGCATAAGAACTGCGGAG | | |
| | AGATGAGTGAGATAGAAGCCAAGGTCAAGTACGTCAAACTCGCACGGTCCCTCCGCAC | | |
| | ATATGGCGTGTCCTTCTTCCTGGTGAAGGAGAAGATGAAAGGCAAGAACAAGCTGGTG | | |
| | CCTCGCCTGCTGGGGATCACCAAAGACTCGGTGATGCGCGTGGATGAGAAGACCAAGG | | |
| | AAGTGCTGCAGGAGTGGCCCCTCACCACCGTCAAGCGCTGGGCAGCCTCACCCAAGAG | | |
| | CTTCACACTGGATTTTGGGGAGTATCAGGAAAGCTACTATTCAGTACAAACCACCGAG | | |
| | GGAGAGCAGATATCCCAGCTGATTGCAGGCTACATTGACATCATCCTGAAAAAGGGAA | | |
| | CATACGTGACATCTGTGGGGTCTCCTCATTGCACTCCACATGGCTGGTGTTCTCTCAG | | |
| | TGACCAAACCACTTTTCCCGGCAGGTCCACCATCTTGCAGCAGCAGTTCAACCGGACC | | |
| | GGGAAGGCAGAGCACGGCTCAGTGGCGCTGCCGGCCGTGATGCGCTCGGGCTCCAGCG | | |
| | GGCCTGAGACCTTCAACGTTGGCAGCATGCCCTCGCCACAGCAGCAGGTCATGGTTGG | | |
| | GCAGATGCACCGAGGCCACATGCCGCCACTGACCTCAGCCCAGCAGGCCCTGATGGGG | | |
| | ACCATCAACACAAGCATGCACGCCGTCCAGCAGGCCCAGGATGATCTCAGTGAGCTCG | | |
| | ACTCGCTGCCACCTCTCGGCCAGGATATGGCATCTAGGGTATGGGTTCAGAACAAAGT | | |
| | CGACGAATCCAAACACGAAATCCATTCTCAAGTTGATGCTATCACGGCCGGAACGGCT | | |
| | TCAGTTGTTAACCTCACAGCTGGTGACCCTGCAGACACTGACTACACAGCTGTGGGAT | | |
| | GTGCGATCACCACTATTTCTTCCAACCTGACGGAGATGTCCAAGGGTGTGAAGCTATT | | |
| | GGCCGCCCTCATGGATGATGAGGTGGGCAGCGGGGAGGACTTGCTCAGAGCTGCCAGG | | |
| | ACCCTCGCTGGGGCGGTGTCAGACTTGCTGAAAGCTGTGCAGCCTACTTCTGGAGAGC | | |
| | CTCGACAGACAGTTTTGACTGCTGCTGGCAGCATCGGACAAGCCAGTGGGGATCTTCT | | |
| | GAGACAGATTGGAGAGAATGAGACTGATGAGCGATTCCAGGATGTTTTAATGAGTTTG | | |
| | GCCAAAGCTGTTGCCAATGCAGCTGCCATGTTGGTACTAAAGGCAAAGAATGTTGCCC | | |
| | AAGTGGCCGAAGACACTGTCCTACAGAACAGGGTAATTGCTGCTGCCACCCAGTGTGC | | |
| | CCTCTCCACCTCCCAGCTTGTGGCATGTGCCAAGGTTGTGAGCCCCACTATTAGCTCC | | |
| | CCTGTGTGCCAGGAGCAGCTGATTGAAGCAGGGAAGCTGGTGGACCGCTCGGTGGAGA | | |
| | ACTGTGTCCGTGCCTGCCAGGCGGCCACTACCGATAGTGAGCTCCTGAAGCAGGTCAG | | |
| | CGCAGCGGCCAGCGTGGTCAGCCAGGCCCTCCATGATCTCCTGCAGCATGTGCGGCAG | | |
| | TTTGCCAGCCGAGGCGAGCCCATCGGCCGCTACGACCAGGCTACTGACACCATCATGT | | |
| | GTGTCACCGAGAGCATCTTCAGCTCCATGGGTGACGCTGGTGAAATGGTGCGCCAGGC | | |
| | GCGGGTTCTGGCCCAAGCCACATCAGACCTCGTCAATGCCATGAGGTCAGATGCAGAA | | |
| | GCCGAAATCGACATGGAGAATTCAAAGAAGCTCCTGGCAGCAGCAAAACTCTTAGCTG | | |
| | ACTCCACTGCTCGCATGGTGGAAGCTGCAAAGGGGGCTGCAGCCAACCCAGAGAATGA | | |
| | GGACCAGCAGCAAAGGCTGAGAGAAGCTGCAGAAGGCCTCCGGGTAGCAACCAACGCA | | |
| | GCTGCCCAGAATGCTATTAAGAAAAAAATTGTCAACCGACTGGAGGTTGCAGCCAAGC | | |
| | AGGCCGCAGCGGCAGCCACACAGACCATCGCCGCCTCCCAGAATGCAGCTGTTTCCAA | | |
| | CAAGAACCCTGCGGCCCAGCAGCAGCTGGTCCAGAGTTGCAAGGCAGTGGCTGATCAC | | |
| | ATCCCTCAGCTGGTCCAGGGAGTGAGGGGGAGCCAAGCTCAAGCTGAAGACCTGAGTG | | |
| | CCCAGCTGGCTCTCATCATCTCCAGCCAGAACTTCCTCCAGCCTGGAAGCAAGATGGT | | |
| | GTCCTCTGCCAAAGCCGCAGTGCCCACCGTGAGTGACCAGGCCGCAGCCATGCAGCTG | | |
| | AGCCAGTGTGCCAAGAACCTGGCCACCAGCTTGGCGGAGCTGCGTACCGCCTCGCAGA | | |
| | AGGCCCATGAAGCTTGTGGTCCGATGGAAATCGATTCAGCTCTGAATACGGTGCAGAC | | |
| | GCTTAAGAATGAACTGCAGGATGCCAAGATGGCAGCCGTGGAGAGCCAGCTGAAGCCA | | |
| | CTTCCAGGGGAAACGCTGGAAAAATGTGCTCAGGACCTGGGAAGCACATCCAAGGCGG | | |
| | TGGGCTCCTCCATGGCACAGCTGCTGACCTGTGCTGCTCAAGGCAACGAACACTACAC | | |
| | AGGGGTGGCTGCTAGAGAGACGGCCCAAGCTCTGAAAACACTGGCCCAGGCCGCCCGT | | |
| | GGAGTGGCTGCATCGACAACCGACCCCGCGGCCGCCCATGCCATGTTAGATTCTGCTC | | |
| | GAGACGTGATGGAGGGCTCCGCCATGCTCATTCAAGAGGCCAAGCAGGCCCTGATTGC | | |
| | ACCTGGAGATGCAGAGCGTCAACAAAGACTGGCTCAGGTGGCTAAAGCCGTCTCACAC | | |
| | TCCTTGAATAACTGCGTAAATTGCCTCCCTGGGCAGAAGGATGTGGACGTGGCCTTGA | | |
| | AGAGCATCGGGGAGTCCAGCAAGAAGCTQCTTGTGGATTCGCTACCTCCAAGCACGAA | | |
| | GCCTTTCCAGGAAGCCCAGAGTGAACTGAACCAGGCAGCAGCTGATCTGAACCAGTCT | | |
| | GCTGGGGAAGTGGTCCATGCCACCCGGGGCCAGAGTGGACAGTTGGCTGCAGCCTCTG | | |
| | GAAAGTTCAGTGATGATTTTGGTGAATTCCTCGATGCTGGCATTGAGATGGCTGGCCA | | |
| | AGCTCAGACAAAAGAAGACCAGATCCAAGTGATAGGGAACCTCAAGAATATCTCGATG | | |
| | GCATCCAGCAAGCTGCTGTTAGCTGCCAAGTCTCTCTCTGTAGATCCAGGAGCTCCCA | | |
| | ATGCGAAAAATCTCCTGGCTGCAGCTGCAAGAGCTGTGACAGAGAGCATCAATCAACT | | |
| | CATCACTCTGTGTACCCAACAAGCTCCGGGCCAGAAAGAGTGCGATAATGCCCTGCGG | | |
| | GAGCTCGAGACTGTGAAGGGGATGTTGGACAATCCTAATGAACCTGTTAGTGACCTCT | | |
| | CTTACTTTGACTGCATTGAGAGTGTGATGGAAAACTCCAAGGTTCTGGGTGAATCGAT | | |
| | GGCAGGGATTTCACAGAATGCCAAGACCGGAGACCTCCCTGCCTTTGGGGAATGTGTG | | |
| | GGGATTGCATCCAAGGCTCTCTGTGGGCTGACAGAGGCTGCAGCCCAGGCTGCATACT | | |
| | TGGTTGGCATCTCTGATCCAAACAGCCAGGCAGGCCACCAGGGCCTGGTGGACCCCAT | | |
| | CCAGTTTGCCAGGGCTAACCAGGCCATCCAGATGGCATGCCAGAACTTGGTGGACCCT | | |
| | GGCAGCAGCCCATCACAGGTCCTGTCAGCCGCCACAATTGTTGCCAAGCACACGTCAG | | |
| | CCTTGTGCAATGCCTGCCGCATCGCCTCATCCAAGACGGCCAACCCAGTAGCCAAGAG | | |
| | GCACTTCGTCCAGTCAGCCAAGGAAGTCGCCAACAGCACTGCCAACCTGGTGAAGACC | | |
| | ATCAAGGCCCTGGATGGGGATTTCTCTGAAGACAACCGCAATAAGTGTCGCATCGCCA | | |
| | CCGCACCCTTGATTGAAGCTGTGGAGAACCTGACAGCGTTCGCCTCAAACCCTGAGTT | | |
| | TGTCAGCATTCCTGCCCAGATCAGCTCCGAGGGTTCCCAGGCACAGGAACCAATCCTG | | |
| | GTCTCAGCCAAGACCATGCTGGAGAGTTCATCGTACCTCATTCGCACTGCACGCTCTC | | |
| | TGGCCATCAACCCCAAAGACCCACCCACCTGGTCTGTACTGGCTGGACATTCCCATAC | | |
| | AGTGTCCGACTCCATCAAGAGTCTCATCACTTCTATCAGGGACAAGGCCCCTGGACAG | | |
| | AGGGAGTGTGATTACTCCATCGATGGCATCAACCGGTGCATCCGGGACATCGAGCAGG | | |
| | CCTCGCTGGCCGCCGTCAGCCAGAGCCTGGCCACGAGGGACGACATCTCTGTGGAGGC | | |
| | CCTGCAGGAGCAGCTGACTTCGGTGGTCCAGGAAATCGGACACCTTATCGATCCCATC | | |
| | GCCACAGCGGCTCGGGGAGAAGCAGCTCAGCTGGGACATAAGGTGACACAACTGGCAA | | |
| | GCTATTTTGAGCCCTTGATCTTAGCCGCAGTTGGTGTGGCCTCCAAGATTCTTGATCA | | |
| | TCAGCAGCAGATGACGGTGCTGGACCAGACCAAGACTCTCGCAGAGTCTGCCTTGCAG | | |
| | ATGTTGTATGCAGCCAAAGAAGGTGGCGGAAACCCCAAGGCACAACACACCCATGACG | | |
| | CCATCACAGAGGCCGCCCAGTTGATGAAGGAAGCCGTGGATGACATCATGGTGACGCT | | |
| | GAACGAAGCTGCCAGTGAAGTGGGGCTGGTTGGGGGCATGGTGGACGCCATTGCAGAA | | |
| | GCCATGAGCAAGCTGGATGAAGGCACTCCTCCAGAACCAAAGGGAACATTTGTCGACT | | |
| | ATCAGACGACTGTGGTTAAATACTCCAAAGCCATTGCGGTGACAGCTCAGGAAATGAT | | |
| | GACTAAGTCGGTTACTAACCCGGAGGAGTTGGGAGGACTGGCTTCACAAATGACCAGT | | |
| | GACTATGGGCACCTGGCTTTCCAGGGCCAGATGGCAGCAGCCACGGCGGAACCAGAGG | | |
| | AGATCGGATTCCAGATTCGCACTCGTGTGCAGGACCTGGGCCACGGCTGTATCTTCCT | | |
| | GGTGCAGAAGGCAGGGGCCCTCCAGGTCTGCCCCACAGACAGCTACACCAAGAGGGAG | | |
| | CTGATCGAATGCGCCCGTGCCGTCACGGAAAAGGTCTCCTTGGTGCTCTCGGCTCTCC | | |
| | AGGCCGGGAACAAAGGAACCCAGGCATGCATTACAGCCGCCACCGCTGTGTCTGGGAT | | |
| | CATTGCCGACCTGGACACCACCATTATGTTTGCAACAGCGGGGACGCTGAATGCAGAG | | |
| | AACAGTGAGACCTTCGCAGACCACAGGGAGAACATTCTCAAGACGGCCAAGGCCTTGG | | |
| | TAGAAGACACGAAACTACTTGTGTCAGGAGCTGCGTCCACTCCTGACAAGCTGGCCCA | | |
| | GGCGGCCCAGTCCTCAGCAGCCACCATCACCCAGCTCGCAGAAGTGGTCAAGCTGGGG | | |
| | GCAGCCAGCCTGGGCTCCGACGACCCCGAGACCCAGGTGGATTTGATCAATGCCATCA | | |
| | AAGATGTGGCCAAGGCCCTTTCTGATCTCATCAGTGCTACCAAGGGAGCTGCCAGCAA | | |
| | GCCAGTGGACGACCCTTCCATGTACCAGCTCAAGGGGGCTGCCAAGGTGATGGTGACC | | |
| | AATGTCACCTCGCTCCTCAAGACTGTAAAGGCAGTGGAGGATGAGGCCACCCGGGGCA | | |
| | CCAGGGCGCTTGAGGCCACAATTGAATGCATAAAGCAGGAGCTTACGGTGTTCCAGTC | | |
| | AAAAGACGTACCTGAAAAGACATCATCACCTGAAGAATCCATAAGGATGACGAAAGGC | | |
| | ATCACCATGGCAACAGCCAAAGCCGTGGCAGCTGGGAACTCATGTAGACAGGAGGACG | | |
| | TGATTGCTACTGCCAACCTGAGCCGGAAAGCCGTGTCAGATATGTTGACGGCTTGCAA | | |
| | GCAAGCATCCTTCCACCCCGATGTCAGTGACGAGGTGAGAACCAGAGCCTTGCGTTTC | | |
| | GGGACGGAGTGCACCCTTGGCTACTTGGACCTCCTGGAGCACGTCTTGGTGATTCTTC | | |
| | AGAAACCAACCCCAGAATTCAAGCAGCAGCTGGCCGCTTTCTCCAAGCGAGTCGCCGG | | |
| | CGCTGTGACAGAGCTCATCCAGGCGGCGGAAGCCATGAAAGGAACAGAGTGGGTGGAT | | |
| | CCAGAAGACCCAACTGTCATTGCAGAAACAGAGTTACTGGGGGCTGCAGCATCCATCG | | |
| | AAGCTGCTGCTAAGAAGTTAGAGCAACTGAAGCCAAGAGCAAAACCAAAACAAGCGGA | | |
| | TGAGACCCTGGACTTTGAGGAACAGATCTTGGAAGCTGCTAAATCCATTGCTGCTGCC | | |
| | ACAAGCGCCCTGGTCAAATCGGCCTCAGCAGCCCAGAGGGAGCTGGTGGCCCAAGGAA | | |
| | AGGTGGGCTCCATCCCTGCCAATGCTGCAGACGACGGACAGTGGTCACAGGGGCTGAT | | |
| | TTCTGCTGCCCGGATGGTGGCGGCTGCGACCAGCAGTCTCTGTGAGGCGGCCAATGCC | | |
| | TCCGTTCAGGGACACGCCAGCGAGGAGAAGCTCATCTCATCTGCCAAGCAGGTCGCCG | | |
| | CTTCCACGGCTCAGCTGCTGGTGGCCTGCAAGGTGAAGGCCGACCAGGATTCAGAGGC | | |
| | CATGAGGCGGCTACAGGCGGCAGGAAATGCTGTGAAAAGAGCCTCAGACAATCTTGTC | | |
| | CGTGCAGCCCAGAAGGCAGCTTTTGGCAAAGCTGATGACGACGATGTTGTAGTGGAAA | | |
| | CCAAGTTTGTGGGGGGCATTGCTCAGATCATCGCCGCCCAGGAAGAAATGCTAAAGAA | | |
| | AGAGCGAGAACTGGAAGAAGCAAGGAAAAAACTGGCCCAAATCCGCCAGCAGCAGTAT | | |
| | AAGTTTTTACCCACCGAGCTGAGGGAAGATGAGGGCTAAAGGTGCGAGCCCAGATGGC | | |
| | GAGCCCCAGGGGATGGCCCTGGCTGAA | | |
| | ORF Start: ATG at 58 | | ORF Stop: TAA at 7693 |
| | SEQ ID NO:76 | 2545 aa | MW at 271692.8 kD |

| | |
|---|---|
| NOV21a, | MVALSLKICVRHCNVVKTMQFEPSTAVYDACRVIRERVPEAQTGQASDYGLFLSDEDP |
| CG57804-01 Protein | |
| Sequence | RKGIWLEAGRTLDYYMLRNGDILEYKKKQRPQKIRMLDGSVKTVMVDDSKTVGELLVT |
| | ICSRIGITNYEEYSLIQETIEEKKEEGTGTLKKDRTLLRDERKMEKLKAKLHTDDDLN |
| | WLDHSRTFREQGVDENETLLLRRKFFYSDQNVDSRDPVQLNLLYVQARDDILNGSHPV |
| | SFEKACEFGGFQAQIQFGPHVEHKHKPGFLDLKEFLPKEYIKQRGAEKRIFQEHKNCG |
| | EMSEIEAKVKYVKLARSLRTYGVSFFLVKEKMKGKNKLVPRLLGITKDSVMRVDEKTK |
| | EVLQEWPLTTVKRWAASPKSFTLDFGEYQESYYSVQTTEGEQISQLIAGYIDIILKKG |
| | TYVTSVGSPHCTPHGWCSLSDQTTFPGRSTILQQQFNRTGKAEHGSVALPAVMRSGSS |
| | GPSTFNVGSMPSPQQQVMVGQMHRGHMPPLTSAQQALMGTINTSMHAVQQAQDDLSEL |
| | DSLPPLGQDMASRVWVQNKVDESKHEIHSQVDAITAGTASVVNLTAGDPADTDYTAVG |
| | CAITTISSNLTEMSKGVKLLAALMDDEVGSGEDLLRAARTLAGAVSDLLKAVQPTSGE |
| | PRQTVLTAAGSIGQASGDLLRQIGENETDERFQDVLMSLAKAVANAAAMLVLKAKNVA |
| | QVAEDTVLQNRVIAAATQCALSTSQLVACAKVVSPTISSPVCQEQLIEAGKLVDRSVE |
| | NCVRACQAATTDSELLKQVSAAASVVSQALHDLLQHVRQFASRGEPIGRYDQATDTIM |
| | CVTESIFSSMGDAGEMVRQARVLAQATSDLVNAMRSDAEAEIDMENSKKLLAAAKLLA |
| | DSTARMVEAAKGAAANPENEDQQQRLREAAEGLRVATNAAAQNAIKKKIVNRLEVAAK |
| | QAAAAATQTIAASQNAAVSNKNPAAQQQLVQSCKAVADHIPQLVQGVRGSQAQAEDLS |
| | AQLALIISSQNFLQPGSKMVSSAKAAVPTVSDQAAAMQLSQCAKNLATSLAELRTASQ |
| | KAHEACGPMEIDSALNTVQTLKNELQDAKMAAVESQLKPLPGETLEKCAQDLGSTSKA |
| | VGSSMAQLLTCAAQGNEHYTGVAARETAQALKTLAQAARGVAASTTDPAAAHAMKDSA |
| | RDVMEGSAMLIQEAKQALIAPGDAERQQRLAQVAKAVSHSLNNCVNCLPGQKDVDVAL |
| | KSIGESSKKLLVDSLPPSTKPFQEAQSELNQAAADLNQSAGEVVHATRGQSGELAAAS |
| | GKFSDDFGEFLDAGIEMAGQAQTKEDQIQVIGNLKNISMASSKLLLAAKSLSVDPGAP |
| | NAKNLLAAAARAVTESINQLITLCTQQAPGQKECDNALRELETVKGMLDNPNEPVSDL |
| | SYFDCIESVMENSKVLGESMAGISQNAKTGDLPAFGECVGIASKALCGLTEAAAQAAY |
| | LVGISDPNSQAGHQGLVDPIQFARANQAIQMACQNLVDPGSSPSQVLSAATIVAKHTS |
| | ALCNACRIASSKTANPVAKRHFVQSAKEVANSTANLVKTIKALDGDFSEDNRNKCRIA |
| | TAPLIEAVENLTAFASNPEFVSIPAQISSEGSQAQEPILVSAKTMLESSSYLIRTARS |
| | LAINPKDPPTWSVLAGHSHTVSDSIKSLITSIRDKAPGQRECDYSIDGINRCIRDIEQ |
| | ASLAAVSQSLATRDDISVEALQEQLTSVVQEIGHLIDPIATAARGEAAQLGHKVTQLA |
| | SYFEPLILAAVGVASKILDHQQQMTVLDQTKTLAESALQMLYAAKEGGGNPKAQHTHD |
| | AITEAAQLMKEAVDDIMVTLNEAASEVGLVGGMVDAIAEAMSKLDEGTPPEPKGTFVD |
| | YQTTVVKYSKAIAVTAQEMMTKSVTNPEELGGLASQMTSDYGHLAFQGQMAAATAEPE |
| | EIGFQIRTRVQDLGHGCIFLVQKAGALQVCPTDSYTKRELIECARAVTEKVSLVLSAL |
| | QAGNKGTQACITAATAVSGIIADLDTTIMFATAGTLNAENSETFADHRENILKTAKAL |
| | VEDTKLLVSGAASTPDKLAQAAQSSAATITQLAEVVKLGAASLGSDDPETQVDLINAI |
| | KDVAKALSDLISATKGAASKPVDDPSMYQLKGAAKVMVTNVTSLLKTVKAVEDEATRG |
| | TRALEATIECIKQELTVFQSKDVPEKTSSPEESIRMTKGITMATAKAVAAGNSCRQED |
| | VIATANLSRKAVSDMLTACKQASFHPDVSDEVRTRALRFGTECTLGYLDLLEHVLVIL |
| | QKPTPEFKQQLAAFSKRVAGAVTELIQAAEAMKGTEWVDPEDPTVIAETELLGAAASI |
| | EAAAKKLEQLKPRAKPKQADETLDFEEQILEAAKSIAAATSALVKSASAAQRELVAQG |
| | KVGSIPANAADDGQWSQSLISAARMVAAATSSLCEAANASVQGHASEEKLISSAKQVA |
| | ASTAQLLVACKVKADQDSEAMRRLQAAGNAVKRASDNLVRAAQKAAFGKADDDDVVVE |
| | TKFVGGIAQIIAAQEEMLKKERELEEARKKLAQIRQQQYKFLPTELREDEG |

Further analysis of the NOV21a protein yielded the following properties shown in Table 21B.

TABLE 21B
Protein Sequence Properties NOV21a

| | |
|---|---|
| PSort | 0.5964 probability located in mitochondrial matrix space; |
| analysis: | 0.3037 probability located in mitochondrial inner membrane; |
| | 0.3037 probability located in mitochondrial intermembrane |
| | space; 0.3037 probability located in mitochondrial outer |
| | membrane |
| SignalP | No Known Signal Sequence Predicted |
| analysis: | |

A search of the NOV21a protein against the Geneseq database, a proprietary database that contains sequences published in patents and patent publication, yielded several homologous proteins shown in Table 21C.

TABLE 21C
Geneseq Results for NOV21a

| | | | Identities/ | |
|---|---|---|---|---|
| | | NOV21a | Similarities | |
| | Protein/ | Residues/ | for the | |
| Geneseq | Organism/Length | Match | Matched | Expect |
| Identifier | [Patent #, Date] | Residues | Region | Value |
| AAB41087 | Human ORFX | 1 . . . 2543 | 1913/2546 | 0.0 |
| | ORF851 poly- | | (75%) | |
| | peptide sequence | 1 . . . 2540 | 2231/2546 | |
| | SEQ ID NO: | | (87%) | |
| | 1702—Homo | | | |
| | sapiens, | | | |
| | 2541 aa. | | | |
| | [WO200058473- | | | |
| | A2, Oct. 5, 2000] | | | |
| AAM39312 | Human poly- | 1381 . . . 2545 | 1161/1165 | 0.0 |
| | peptide SEQ ID | | (99%) | |
| | NO 2457—Homo | 1 . . . 1165 | 1163/1165 | |
| | sapiens, 1165 aa. | | (99%) | |
| | [WO200153312- | | | |
| | A1, Jul. 26, 2001] | | | |
| AAM79794 | Human protein | 1378 . . . 2545 | 1156/1168 | 0.0 |
| | SEQ ID NO | | (98%) | |
| | 3440—Homo | 10 . . . 1177 | 1160/1168 | |
| | sapiens, 1177 aa. | | (98%) | |
| | [WO200157190- | | | |
| | A2, Aug. 9, | | | |
| | 2001] | | | |
| AAM41098 | Human poly- | 1378 . . . 2545 | 1156/1168 | 0.0 |
| | peptide SEQ ID | | (98%) | |
| | NO 6029—Homo | 10 . . . 1177 | 1160/1168 | |
| | sapiens, 1177 aa. | | (98%) | |
| | [WO200153312- | | | |
| | A1, Jul. 26, 2001] | | | |
| AAM41079 | Human poly- | 1378 . . . 2545 | 1156/1168 | 0.0 |
| | peptide SEQ ID | | (98%) | |
| | NO 6010—Homo | 10 . . . 1177 | 1160/1168 | |
| | sapiens, 1177 aa. | | (98%) | |
| | [WO200153312- | | | |
| | A1, Jul. 26, 2001] | | | |

In a BLAST search of public sequence databases, the NOV21a protein was found to have homology to the proteins shown in the BLASTP data in Table 21D.

TABLE 21D
Public BLASTP Results for NOV21a

| | | | Identities/ | |
|---|---|---|---|---|
| | | NOV21a | Similarities | |
| Protein | | Residues/ | for the | |
| Accession | Protein/ | Match | Matched | Value |
| Number | Organism/Length | Residues | Portion | Expect |
| Q9Y490 | Talin—Homo | 1 . . . 2543 | 1910/2546 | 0.0 |
| | sapiens (Human), | | (75%) | |
| | 2541 aa. | 1 . . . 2540 | 2230/2546 | |
| | | | (87%) | |
| P26039 | Talin—Mus | 1 . . . 2543 | 1907/2546 | 0.0 |
| | musculus | | (74%) | |
| | (Mouse), 2541 aa. | 1 . . . 2540 | 2230/2546 | |
| | | | (86%) | |
| Q9UPX3 | KIAA1027 | 853 . . . 2543 | 1262/1694 | 0.0 |
| | PROTEIN— | | (74%) | |
| | Homo (fragment). | 1 . . . 1694 | 1483/1694 | |
| | | | (87%) | |
| Q9VSL8 | CG6831 | 1 . . . 2532 | 1197/2563 | 0.0 |
| | PROTEIN | | (46%) | |
| | (TALIN)— | 1 . . . 2534 | 1707/2563 | |
| | Drosophila | | (65%) | |
| | melanogaster | | | |
| | (Fruit fly), | | | |
| | 2836 aa. | | | |
| Q9Y4G6 | KIAA0320 | 1597 . . . 2545 | 947/949 | 0.0 |
| | PROTEIN— | | (99%) | |
| | Homo sapiens | 1 . . . 949 | 948/949 | |
| | (Human), 949 aa | | (99%) | |
| | (fragment). | | | |

PFam analysis predicts that the NOV21a protein contains the domains shown in the Table 21E.

TABLE 21E
Domain Analysis of NOV21a

| | | Identities/ | |
|---|---|---|---|
| | | Similarities | |
| | NOV21a | for the | Expect |
| Pfam Domain | Match Region | Matched Region | Value |
| ubiquitin: domain 1 of 1 | 64 . . . 88 | 8/27 (30%) | 4.3 |
| | | 20/27 (74%) | |
| Band_41: domain 1 of 1 | 123 . . . 316 | 67/211 (32%) | 1.3e−92 |
| | | 172/211 (82%) | |
| IRS: domain 1 of 1 | 312 . . . 404 | 19/109 (17%) | 1.2 |
| | | 46/109 (42%) | |
| I_LWEQ: domain 1 of 5 | 674 . . . 768 | 31/98 (32%) | 11 |
| | | 59/98 (60%) | |
| transport_prot: domain | 667 . . . 814 | 24/182 (13%) | 10 |
| 1 of 1 | | 88/182 (48%) | |
| I_LWEQ: domain 2 of 5 | 852 . . . 894 | 18/47 (38%) | 2.4e+02 |
| | | 31/47 (66%) | |
| Vinculin: domain 1 of 1 | 860 . . . 903 | 12/48 (25%) | 1.3 |
| | | 30/48 (62%) | |
| I_LWEQ: domain 3 of 5 | 925 . . . 984 | 21/62 (34%) | 5.9e+04 |
| | | 37/62 (60%) | |
| TP_methylase: domain | 861 . . . 1036 | 26/226 (12%) | 8 |
| 1 of 1 | | 105/226 (46%) | |
| Apolipoprotein: domain | 981 . . . 1229 | 48/288 (17%) | 3.5 |
| 1 of 1 | | 141/288 (49%) | |
| CAP: domain 1 of 1 | 917 . . . 1354 | 94/557 (17%) | 4.4 |
| | | 209/557 (38%) | |
| I_LWEQ: domain 4 of 5 | 1529 . . . 1545 | 10/17 (59%) | 56 |
| | | 13/17 (76%) | |
| STAT: domain 1 of 1 | 1660 . . . 1821 | 35/211 (17%) | 8.2 |
| | | 95/211 (45%) | |
| LEA: domain 1 of 1 | 1768 . . . 1834 | 15/76 (20%) | 7 |
| | | 42/76 (55%) | |
| Histone_HNS: domain | 2232 . . . 2356 | 29/143 (20%) | 3.7 |
| 1 of 1 | | 63/143 (44%) | |
| I_LWEQ: domain 5 of 5 | 2345 . . . 2536 | 100/202 (50%) | 2e−101 |
| | | 183/202 (91%) | |

### Example 22

The NOV22 clone was analyzed, and the nucleotide and predicted polypeptide sequences are shown in Table 22A.

TABLE 22A
NOV22 Sequence Analysis

| | SEQ ID NO:77 | 2214 bp | |
|---|---|---|---|
| NOV22a, | ATTCCTCCCTGCCCCTCGTGCAGCCGCTGCCATGGCCCAGACACTGCAGATGGAGATC | | |
| CG57551-01 DNA Sequence | | | |
| | CCGAACTTCGGCAACAGCATCCTGGAGTGCCTCAATGAACAGCGGCTGCAGGGCCTGT | | |
| | ACTGTGACGTGTCAGTGGTGGTCAAGGGCCATGCCTTCAAGGCCCACCGGGCCGTGCT | | |
| | TGCTGCCAGCAGCTCCTACTTCCGGGACCTGTTCAACAACAGCCGCAGCGCCGTGGTG | | |
| | GAGCTGCCGCCGGCTGTGCAGCCCCAGTCTTTCCAGCAGATCCTCAGCTTCTGCTACA | | |
| | CGGGCCGGCTGAGCATGAACGTGGGCGACCAGTTCCTGCTCATGTACACGGCTGGCTT | | |
| | CCTGCAGATCCAGGAGATCATGGAGAAGGGCACCGAGTTCTTCCTCAAGGTGAGCTCC | | |
| | CCGAGCTGCGACTCCCAGGGCCTGCATGCGGAGGAGGCCCCATCGTCGGAGCCCCAGA | | |
| | GCCCCGTGGCGCAGACATCGGGCTGGCCAGCCTGTAGCACCCCGCTGCCCCTCGTGTC | | |
| | GCGGGTGAAGACGGAGCAGCAGGAGTCGGACTCCGTGCAGTGCATGCCCGTGGCCAAG | | |
| | CGGCTGTGGGACAGTGGCCAGAAGGAGGCTGGGGGCGGCGGCAATGGCAGCCGCAAGA | | |
| | TGGCCAAGTTCTCCACGCCGGACCTGGCTGCCAACCGGCCTCACCAGCCCCCGCCACC | | |
| | CCAACAGGCTCCGGTGGTGGCAGCAGCCCAGCCCGCCGTGGCTGCGGGAGCAGGGCAG | | |
| | CCAGCCGGTGGGGTGGCAGCAGCAGGGGGTGTGGTGAGTGGGCCCAGCACGTCGGAGC | | |
| | GGACCAGCCCAGGCACCTCAAGCGCCTACACCAGCGACAGCCCTGGCTCCTACCACAA | | |
| | TGAGGAGGACGAGGAGGAGGATGGTGGCGAGGAGGGCATGGATGAGCAGTACCGGCAG | | |
| | ATCTGCAACATGTACACCATGTACAGCATGATGAACGTCGGCCAGACAGCCGAGAAGG | | |
| | TGGAGGCCCTCCCGGAGCAGGTAGCCCCCGAGTCCCGAAATCGCATCCGGGTTCGGCA | | |
| | AGACCTGGCGTCTCTCCCGGCTGAACTTATCAACCAGATTGGGAACCGCTGCCACCCC | | |
| | AAGCTCTACGACGAGGGCGACCCCTCTGAGAAGCTGGAGCTGGTGACAGGCACCAACG | | |
| | TGTACATCACAAGGGCGCAGCTGATGAACTGCCACGTCAGCGCAGGCACGCGGCACAA | | |
| | GGTCCTACTGCGGCGGCTCCTGGCCTCCTTCTTTGACCGGAACACGCTGGCCAACAGC | | |
| | TGCGGCACCGGCATCCGCTCTTCTACCAACGATCCCCGTCGGAAGCCCCTGGACAGCC | | |
| | GCGTGCTCCACGCTGTCAAGTACTACTGCCAGAACTTCGCCCCCAACTTCAAGGAGAG | | |
| | CGAGATGAATGCCATCGCGGCCGACATGTGCACCAACGCCCGCCGCGTCGTGCGCAAG | | |
| | AGCTGGATGCCCAAGGTCAAGGTGCTCAAGGCTGAGGATGACGCCTACACCACCTTCA | | |
| | TCAGTGAAACGGGCAAGATCGAGCCGGACATGATGGGTGTGGAGCATGGCTTCGAGAC | | |
| | CGCCAGCCACGAGGGCGAGGCGGGTCCCATCGCTGAAGCCCTGCAGTAACCCGCCCAG | | |
| | CCTCCCGCGGGGCCGCACACTTCCCCTCCCAACACACACACACACCTGCCATCTTGGT | | |
| | CATGAGCTACTGTCTGTCCCTCCCCAGGACCCGCGGTGGGTGCTGCATGTTCCCGGCC | | |
| | CTCTGCCCCTCCTGTCCTACCCCCTTTCCCCACCGAGAGCTGGGCCGGGAGAGGACCG | | |
| | CAGGGCAGGTGGCGTGAGGTCCGTGTTGCCTTCTTTAACACACACTCGTGCAGTGGGG | | |
| | GAGTTCTGGCTCCCCAACCTAACCCCTAGCCGTCATCTCCACACTCACCAGGCCCACC | | |
| | AGGGGAGGGGGCTGGCCTGGGGGTCTTGGGAAGGCCCCTCCCCAGGCCTTAGGCCACC | | |
| | TCGCGGAAGCCTTCAGCCTCCGCCCCTCACTGCAGCCCCTTGGGACTTGAGGGGGGCC | | |
| | CCAGGGGTTCTCAGGACCCCTCCCACCACCTCCCAGTGCTTCCACGTCTCCAAAAGCG | | |
| | CCTTCCTGTCACCCTCGTCTATCCCTGCGCCTGGGGGCTGGGGTAGGCGAGGCCGTGG | | |
| | GGACTACCCATTTTATAGCTGGGGAAACAGGCTCCGAGAAATTGCACAACCGACCTCA | | |
| | GGTGGCCGGC | | |
| | ORF Start: ATG at 32 | | ORF Stop: TAA at 1613 |
| | SEQ ID NO:78 | 527 aa | MW at 57283.8 kD |

| | |
|---|---|
| NOV22a, | MAQTLQMEIPNFGNSILECLNEQRLQGLYCDVSVVVKGHAFKAHRAVLAASSSYFRDL |
| CG57551-01 Protein Sequence | |
| | FNNSRSAVVELPAAVQPQSFQQILSFCYTGRLSMNVGDQFLLMYTAGFLQIQEIMEKG |
| | TEFFLKVSSPSCDSQGLHAEEAPSSEPQSPVAQTSGWPACSTPLPLVSRVKTEQOESD |
| | SVQCMPVAKRLWDSGQKEAGGGGNGSRKMAKFSTPDLAANRPHQPPPPQQAPVVAAAQ |
| | PAVAAGAGQPAGGVAAAGGVVSGPSTSERTSPGTSSAYTSDSPGSYHNEEDEEEDGGE |
| | EGMDEQYRQICNMYTMYSMMNVGQTAEKVEALPEQVAPESRNRIRVRQDLASLPAELI |
| | NQIGNRCHPKLYDEGDPSEKLELVTGTNVYITRAQLMNCHVSAGTRHKVLLRRLLASF |
| | FDRNTLANSCGTGIRSSTNDPRRKPLDSRVLHAVKYYCQNFAPNFKESEMNAIAADMC |
| | TNARRVVRKSWMPKVKVLKAEDDAYTTFISETGKIEPDMMGVEHGFETASHEGEAGPI |
| | AEALQ |

Further analysis of the NOV22a protein yielded the following properties shown in Table 22B.

TABLE 22B
Protein Sequence Properties NOV22a

| | |
|---|---|
| PSort | 0.6000 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | NOV22a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAB41621 | Human ORFX | 300 . . . 527 | 228/228 | e−131 |
| | ORF1385 poly- | | (100%) | |
| | peptide sequence | 1 . . . 228 | 228/228 | |
| | SEQ ID NO: | | (100%) | |
| | 2770—Homo | | | |
| | sapiens, 228 aa. | | | |
| | [WO200058473- | | | |
| | A2, Oct. 5, 2000] | | | |
| ABB17117 | Human nervous | 409 . . . 501 | 64/94 | 7e−29 |
| | system related | | (68%) | |
| | polypeptide SEQ | 1 . . . 93 | 73/94 | |
| | ID NO 5774— | | (77%) | |
| | Homo sapiens, | | | |
| | 190 aa. | | | |
| | [WO200159063- | | | |
| | A2, Aug. 16, | | | |
| | 2001] | | | |
| AAG78615 | Human zinc | 5 . . . 159 | 62/164 | 2e−25 |
| | finger tran- | | (37%) | |
| | scription factor | 7 . . . 170 | 92/164 | |
| | BioZFTF45— | | (55%) | |
| | Homo sapiens, | | | |
| | 413 aa. | | | |
| | [CN1299825-A, | | | |
| | Jun. 20, 2001] | | | |
| AAY73351 | HTRM clone | 7 . . . 291 | 83/291 | 8e−18 |
| | 1484257 protein | | (28%) | |
| | [WO9957144-A2, | 1 . . . 277 | 124/291 | |
| | Nov. 11, 1999] | | (42%) | |
| AAM41058 | Human poly- | 7 . . . 291 | 84/295 | 2e−17 |
| | peptide SEQ ID | | (28%) | |
| | NO 5989—Homo | 2 . . . 271 | 123/295 | |
| | sapiens, 804 aa. | | (41%) | |
| | [WO200153312- | | | |
| | A1, Jul. 26, 2001] | | | |

In a BLAST search of public sequence databases, the NOV22a protein was found to have homology to the proteins shown in the BLASTP data in Table 22D.

TABLE 22D
Public BLASTP Results for NOV22a

| | | NOV22a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96RE7 | NAC1 | 1 . . . 527 | 526/527 (99%) | 0.0 |
| | PROTEIN— | 1 . . . 527 | 526/527 (99%) | |
| | Homo sapiens | | | |
| | (Human), 527 aa. | | | |
| O35260 | NAC-1 | 1 . . . 527 | 462/530 (87%) | 0.0 |
| | PROTEIN— | 1 . . . 514 | 475/530 (89%) | |
| | Rattus norvegicus | | | |
| | (Rat), 514 aa. | | | |
| Q9CZ72 | 4930511N13R1K | 1 . . . 527 | 462/530 (87%) | 0.0 |
| | PROTEIN—Mus | 1 . . . 514 | 476/530 (89%) | |
| | musculus | | | |
| | (Mouse), 514 aa. | | | |
| Q96BF6 | SIMILAR TO | 1 . . . 501 | 289/522 (55%) | e−140 |
| | RIKEN CDNA | 1 . . . 478 | 335/522 (63%) | |
| | 0610020I02 | | | |
| | GENE—Homo | | | |
| | sapiens (Human), | | | |
| | 587 aa. | | | |
| AAH22103 | RIKEN CDNA | 1 . . . 485 | 281/502 (55%) | e−139 |
| | 0610020I02 | 1 . . . 459 | 327/502 (64%) | |
| | GENE—Mus | | | |
| | musculus | | | |
| | (Mouse), 586 aa. | | | |

PFam analysis predicts that the NOV22a protein contains the domains shown in the Table 22E.

TABLE 22E
Domain Analysis of NOV22a

| | | Identities/ | |
|---|---|---|---|
| | NOV22a | Similarities for | Expect |
| Pfam Domain | Match Region | the Matched Region | Value |
| BTB: domain 1 of 1 | 14 . . . 124 | 40/143 (28%) | 6.2e−23 |
| | | 88/143 (62%) | |

### Example 23

The NOV23 clone was analyzed, and the nucleotide and predicted polypeptide sequences are shown in Table 23A.

TABLE 23A
NOV23 Sequence Analysis

| | SEQ ID NO:79 | 1497 bp | |
|---|---|---|---|
| NOV23a, | b ATGGCCACTGCACAGGTGGAACTGGTGCAGGGTGGTCCCCGGGCTCCAGTAGGGGAGA | | |
| CG57411-01 DNA Sequence | | | |
| | AGCTGGAGCTCGTCCTGTCGAACCTGCAGGCAGACGTCCTGGAGTTGCTGCTGGAGTT | | |
| | TGTCTACACGGGCTCCCTGGTCATCGACTCGGCCAACGCCAAGACACTGCTGGAGGCG | | |
| | GCCAGCAAGTTCCAGTTCCACACCTTCTGCAAAGTCTGCGTGTCCTTTCTCGAGAAGC | | |
| | AGCTGACGGCCAGCAACTGCCTGGGCGTGCTGGCCATGGCCGAGGCCATGCAGTGCAG | | |
| | CGAGCTCTACCACATGGCCAAGGCCTTCGCGCTGCAGATCTTCCCCGAGGTGGCCGCC | | |
| | CAGGAGGAGATCCTCAGCATCTCCAAGGACGACTTCATCGCCTACGTCTCCAACGACA | | |
| | GCCTCAACACCAAGGCTGAGGAGCTGGTGTACGAGACAGTCATCAAGTGGATCAAGAA | | |
| | GGACCCCGCGACACGCACACAGCTGCAGTACGCGGCTGAGCTCCTGGCCGTGGTCCGC | | |
| | CTCCCCTTCATCCACCCCAGCTACCTGCTCAATGTGGTTGACAATGAAGAGCTGATCA | | |
| | AGTCATCAGAAGCCTGCCGGGACCTGGTGAACGAGGCCAAACGCTACCATATGCTGCC | | |
| | CCACGCCCGCCAGGAGATGCAGACGCCCCGAACCCGGCCGCGCGTCCCTGCAGGTGTG | | |
| | GCTGAGGTCATCGTCTTGGTTGGGGGCCGTCAGATGGTGGGGATGACCCAGCGCTCGC | | |
| | TGGTGGCCGTCACCTGCTGGAACCCGCAGAACAACAAGTGGTACCCCTTGGCCTCGCT | | |
| | GCCCTTCTATGACCGCGAGTTCTTCAGTGTAGTGAGTGCAGGGGACAACATCTACCTC | | |
| | TCAGGTGGGATGGAATCAGGGGTGACGCTGGCTGATGTCTGGTGCTACATGTCCCTGC | | |
| | TTGATAACTGGAACCTCGTCTCCAGAATGACAGTCCCCCGCTGTCGGCACAATAGCCT | | |
| | CGTCTACGATGGGAAGATTTACACCCTCGGGGGACTTGGCGTGGCAGGCAACGTGGAC | | |
| | CACGTGGAGGTCCCTGCAGGTGTGGCTGAGGTCATCGTCTTGGTTGGGGGCCGTCAGA | | |
| | TGGTGGGGATGACCCAGCGCTCGCTGGTGGCCGTCACCTGCTGGAACCCGCAGAACAA | | |
| | CAAGTGGTACCCCTTGGCCTCGCTGGGTGGGATGGAATCAGGGGTGACGCTGGCTGAT | | |
| | GTCTGGTGCTACATGTCCCTGCTTGATAACTGGAACCTCGTCTCCAGAATGACAGTCC | | |
| | CCCGCTGTCGGCACAATAGCCTCGTCTACGATGGGAAGATTTACACCCTCGGGGGACT | | |
| | TGGCGTGGCAGGCAACGTGGACCACGTGGAGGCCTACGAGCCCACAACCAACACATGG | | |
| | ACCCTCCTCCCCCACATGCCCTGCCCTGTGTTCAGACACGGCTGCGTCGTGATAAAGA | | |
| | AATATATTCAAAGCGGCTGACATCAGCAGAAAGCCCACGATAAGACT | | |
| | ORF Start: ATG at 1 | | ORF Stop: TGA at 1468 |
| | SEQ ID NO:80 | 489 aa | MW at 54208.2 kD |

| | |
|---|---|
| NOV23a, | MATAQVELVQGGPRAPVGEKLELVLSNLQADVLELLLEFVYTGSLVIDSANAKTLLEA |
| CG57411-01 Protein Sequence | |
| | ASKFQFHTFCKVCVSFLEKQLTASNCLGVLAMAEAMQCSELYHMAKAFALQIFPEVAA |
| | QEEILSISKDDFIAYVSNDSLNTKAEELVYETVIKWIKKDPATRTQLQYAAELLAVVR |
| | LPFIHPSYLLNVVDNEELIKSSEACRDLVNEAKRYHMLPHARQEMQTPRTRPRVPAGV |
| | AEVIVLVGGRQMVGMTQRSLVAVTCWNPQNNKWYPLASLPFYDREFFSVVSAGDNIYL |
| | SGGMESGVTLADVWCYMSLLDNWNLVSRMTVPRCRHNSLVYDGKIYTLGGLGVAGNVD |
| | HVEVPAGVAEVIVLVGGRQMVGMTQRSLVAVTCWNPQNNKWYPLASLGGMESGVTLAD |
| | VWCYMSLLDNWNLVSRMTVPRCRHNSLVYDGKIYTLGGLGVAGNVDHVEAYEPTTNTW |
| | TLLPHMPCPVFRHGCVVIKKYIQSG |

Further analysis of the NOV23a protein yielded the following properties shown in Table 23B.

TABLE 23B
Protein Sequence Properties NOV23a

| | |
|---|---|
| PSort | 0.6500 probability located in cytoplasm; 0.2271 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV23a protein against the Geneseq database, a proprietary database that contains sequences published in patents and patent publication, yielded several homologous proteins shown in Table 23C.

TABLE 23C
Geneseq Results for NOV23a

| | | NOV23a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAB40940 | Human ORFX | 19 . . . 351 | 317/333 (95%) | e−180 |
| | ORF704 poly- | 4 . . . 334 | 320/333 (95%) | |
| | peptide sequence | | | |
| | SEQ ID NO: | | | |
| | 1408—Homo | | | |
| | sapiens, 335 aa. | | | |
| | [WO200058473- | | | |
| | A2, Oct. 5, 2000] | | | |
| AAM38711 | Human poly- | 22 . . . 472 | 151/488 (30%) | 2e−61 |
| | peptide SEQ ID | 78 . . . 559 | 222/488 (44%) | |
| | NO 1856—Homo | | | |
| | sapiens, 574 aa. | | | |
| | [WO200153312- | | | |
| | A1, Jul. 26, 2001] | | | |
| AAB43090 | Human ORFX | 22 . . . 468 | 150/491 (30%) | 3e−59 |
| | ORF2854 poly- | 9 . . . 487 | 241/491 (48%) | |
| | peptide sequence | | | |
| | SEQ ID NO: | | | |
| | 5708—Homo | | | |
| | sapiens, 506 aa. | | | |
| | [WO200058473- | | | |
| | A2, Oct. 5, 2000] | | | |
| AAM38956 | Human poly- | 22 . . . 468 | 149/491 (30%) | 1e−58 |
| | peptide SEQ ID | 90 . . . 568 | 240/491 (48%) | |
| | NO 2101—Homo | | | |
| | sapiens, 587 aa. | | | |
| | [WO200153312- | | | |
| | A1, Jul. 26, 2001] | | | |
| AAM94018 | Human stomach | 25 . . . 470 | 148/490 (30%) | 3e−56 |
| | cancer expressed | 76 . . . 553 | 231/490 (46%) | |
| | polypeptide SEQ | | | |
| | ID NO 106— | | | |
| | Homo sapiens, | | | |
| | 568 aa. | | | |
| | [WO200109317- | | | |
| | A1, Feb. 8, 2001] | | | |

In a BLAST search of public sequence databases, the NOV23a protein was found to have homology to the proteins shown in the BLASTP data in Table 23D.

TABLE 23D
Public BLASTP Results for NOV23a

| | | NOV23a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96CT2 | HYPO- | 19 . . . 489 | 390/507 (76%) | 0.0 |
| | THETICAL | 203 . . . 707 | 406/507 (79%) | |
| | 76.8 KDA | | | |
| | PROTEIN— | | | |
| | Homo sapiens | | | |
| | (Human), 707 aa | | | |
| | (fragment). | | | |
| Q96PW7 | KIAA1921 | 19 . . . 489 | 390/507 (76%) | 0.0 |
| | PROTEIN— | 41 . . . 545 | 406/507 (79%) | |
| | Homo sapiens | | | |
| | (Human), 545 aa | | | |
| | (fragment). | | | |
| Q96BF0 | SIMILAR | 19 . . . 351 | 329/333 (98%) | 0.0 |
| | TO HYPO- | 172 . . . 502 | 330/333 (98%) | |
| | THETICAL | | | |
| | PROTEIN | | | |
| | FLJ14106— | | | |
| | Homo sapiens | | | |
| | (Human), 503 aa. | | | |
| Q9D5K3 | 4930429H24RIK | 33 . . . 485 | 165/492 (33%) | 2e−66 |
| | PROTEIN— | 1 . . . 477 | 248/492 (49%) | |
| | Mus musculus | | | |
| | (Mouse), 484 aa. | | | |
| Q9UH77 | Kelch-like protein | 22 . . . 468 | 150/491 (30%) | 1e−58 |
| | 3—Homo sapiens | 90 . . . 568 | 241/491 (48%) | |
| | (Human), 587 aa. | | | |

PFam analysis predicts that the NOV23a protein contains the domains shown in the Table 23E.

TABLE 23E
Domain Analysis of NOV23a

| | | Identities/ | |
|---|---|---|---|
| | NOV23a | Similarities for | Expect |
| Pfam Domain | Match Region | the Matched Region | Value |
| BTB: domain 1 of 1 | 4 . . . 79 | 24/143 (17%) | 3.7 |
| | | 53/143 (37%) | |
| Kelch: domain 1 of 4 | 223 . . . 272 | 9/50 (18%) | 0.94 |
| | | 28/50 (56%) | |
| Kelch: domain 2 of 4 | 275 . . . 320 | 11/47 (23%) | 0.016 |
| | | 27/47 (57%) | |
| Kelch: domain 3 of 4 | 322 . . . 396 | 14/75 (19%) | 3.3e−05 |
| | | 44/75 (59%) | |
| Kelch: domain 4 of 4 | 426 . . . 471 | 19/47 (40%) | 7.2e−10 |
| | | 35/47 (74%) | |

### Example 24

The NOV24 clone was analyzed, and the nucleotide and predicted polypeptide sequences are shown in Table 24A.

TABLE 24A
NOV24 Sequence Analysis

| | SEQ ID NO:81 | 4268 bp | |
|---|---|---|---|
| NOV24a, | ATGACCTGGGACACAGCTCTCTGGACCTCAGTTTTTCTGATTGGGCTCCTTCCTACCC | | |
| CG57399-01 DNA Sequence | | | |
| | TTGGTTTCGCTAATTGCATCCTCCAGACTTCTGGTAAAATGTGTACTTTAAGAGGTAG | | |
| | ATACCCCCAGCCCCCACAACCACCTCTCTGCTTGTCTCCCCTAGTCCACCAGCTCCGA | | |
| | CCAGCAGACATCAAAGTGGTGGCCGCCCTGGGTAATGATGAAACCTTCCAGGAAAGTG | | |
| | GTGCAGGGCAGCTAAGTGAGCCTGACCCCAGGCAGTGGTCCTGGCCACAGGCCTGCTT | | |
| | GCCTGGGGTAAAAAAGGAAATGCAAGATGTGGTAGGTGAGAGAACGCCGAGCCGTCGC | | |
| | CGCAGCCTCCGCCGCCGAGAAGCCCTTGTTCCCGCTGCTGGGAAGGAGAGTCTGTGCC | | |
| | GACAAGATATTTTCATTTCCTTGTTGGAAATTATCAAGCATTTTCCTCCCTCCCCTCA | | |
| | GGACATCAACCTGGAGAAAGACTGGAAGCTGGTCACACTCTTCATTGGGGTCAACGAC | | |
| | TTGTGTCATTACTGTCCACTTGTTCAGGGCCCCGTTATAGACCTGGGTGGGATGGATA | | |
| | CCCTCCACTCCCTGCAGCTCCCAAGGGCTTTCGTCAACGTGGTGGAGGTCATGGAGCT | | |
| | GGCTAGCCTGTACCAGGGCCAAGGCGGGAAATGTGCCATGCTGGCAGCTCAGGAAGCC | | |
| | TGGAACAGCCTCCTGGCCTCCAGCAGGTACAGTGAGCAGGAGTCCTTCACCGTGGTTT | | |
| | TCCAGCCTTTCTTCTATGAGACCACCCCATCTGACCCCCGACTCCAGGATTCTACCAC | | |
| | GCTGGCCTGGCATCTCTGGAATAGGATGATGGAGCCAGCAGGAGAGAAAGATGAGCCA | | |
| | TTGAGTGTAAAACACGGGAGGCCAATGAAGTGTCCCTCTCAGGAGAGCCCCTATCTGT | | |
| | TCAGCTACAGAAACAGCAACTACCTGACCAGACTGCAGAAACCCCAAGACAAGCTTGT | | |
| | AAGAGAAGGAGCGGAAATCAGATGTCCTGACAAAGACCCCTCCGATACGGTTCCCACC | | |
| | TCAGTTCATAGGCTGAAGCCGGCTGACATCAACGTAATTGGAGCCCTGGGTGACTCTC | | |
| | TCACGGCAGGCAATGGGGCCGGGTCCACACCTGGGAACGTCTTGGACGTCTTGACTCA | | |
| | GTACCGAGGCCTGTCCTGGAGCGTCGGCGGAGATGAGAACATCGGCACCGTTACCACC | | |
| | CTGGCAGACATCCTCCGGGAATTCAACCCTTCCCTGAAGGGCTTCTCTGTTGGCACTG | | |
| | GGAAAGAAACCAGTCCTAATGCCTTCTTAAACCAGGCTGTGGCAGGAGGCCGAGCTGA | | |
| | GCAGGCCAGGAGGCTGGTGGACCTGATGAAGAATGACACGAGGATACACTTTCAGGAA | | |
| | GACTGGAAGATAATAACCCTGTTTATAGGCGGCAATGACCTCTGTGATTTCTGCAATG | | |
| | ATCTGGTACACTATTCTCCCCAGAACTTCACAGACAACATTGGAAAGGCCCTGGACAT | | |
| | CCTCCATGCTGAGTCTCAGGTTCCTCGGGCATTTGTGAACCTGGTGACGGTGCTTGAG | | |
| | ATCGTCAACCTGAGGGAGCTGTACCAGGAGAAAAAAGTCTACTGCCCAAGGATGATCC | | |
| | TCAGGTCACTGTGTCCCTGTGTCCTGAAGTTTGATGATAACTCAACAGAACTTGCTAC | | |
| | CCTCATCGAATTCAACAAGAAGTTTCAGGAGAAGACCCACCAACTGATTGAGAGTGGG | | |
| | CGATATGACACAAGGGAAGATTTTACTGTGGTTGTGCAGCCGTTCTTTGAAAACGTGG | | |
| | ACATGCCAAAGACCCAGGAAGGATTGCCTGACAACTCTTTCTTCGCTCCTGACTGTTT | | |
| | CCACTTCAGCAGCAAGTCTCACTCCCGAGCAGCCAGTGCTCTCTGGAACAATATGCTG | | |
| | GAGCCTGTTGGCCAGAAGACGACTCGTCATAAGTTTGAAAACAAGATCAATATCACAT | | |
| | GTCCGTCACAGGTCCAGCCGTTTCTGAGGACCTACAAGAACAGCATGCAGGGTCATGG | | |
| | GACCTGGCTGCCATGCAGGGACAGAGCCCCTTCTGCCTTGCACCCTACCTCAGTGCAT | | |
| | GCCCTGAGACCTGCAGACATCCAAGTTGTGGCTGCTCTGGGGGATTCTCTGACCGCTG | | |
| | GCAATGGAATTGGCTCCAAACCAGACGACCTCCCCGATGTCACCACACAGTATCGGGG | | |
| | ACTGTCATACAGTGCAGGAGGGGACGGCTCCCTGGAGAATGTCACCACCTTACCTAGT | | |
| | TCTATCCTTCGCGAGTTTAACAGAAACCTCACAGGCTACGCCGTCGGCACGGGTGATG | | |
| | CCAATGACACGAATGCATTCCTCAATCAAGCTGTTCCCGGAGCAAAGGCTAGGGATCT | | |
| | TATGAGCCAAGTCCAAACTCTGATGCAGAAGATGAAAGATGATCATAGAGTAAATTTC | | |
| | CATGAAGACTGGAAGGTCATCACAGTGCTGATCGGAGGCAGCGATTTATGTGACTACT | | |
| | GCACAGATTCGAATCTGTATTCTGCAGCCAACTTTGTTCACCATCTCCGCAATGCCTT | | |
| | GGACGTCCTCCATAGAGAGGTGCCCAGAGTCCTGGTCAACCTCGTGGACTTCCTGAAC | | |
| | CCCACTATCATGCGGCAGGTGTTCCTGGGAAACCCAGACAAGTGCCCAGTGCAGCAGG | | |
| | CCAGCGTTTTGTGTAACTGCGTTCTGACCCTGCGGGAGAACTCCCAAGAGCTAGCCAG | | |
| | GCTGGAGGCCTTCAGCCGAGCCTACCAGAGCAGCATGCGCGAGCTGGTGGGGTCAGGC | | |
| | CGCTATGACACGCAGGAGGACTTCTCTGTGGTGCTGCAGCCCTTCTTCCAGAACATCC | | |
| | AGCTCCCTGTCCTGCAGGATGGGCTCCCAGATACGTCCTTCTTTGCCCCAGACTGCAT | | |
| | CCACCCAAATCAGAAATTCCACTCCCAGCTGGCCAGACCCCTTTGGACCAATATGCTT | | |
| | GAACCACTTGGAAGCAAAACAGAGACCCTGGACCTGAGAGCAGAGATGCCCATCACCT | | |
| | GTCCCACTCAGAATGAGCCCTTCCTGAGAACCCCTCGGAATAGTAACTACACGTACCC | | |
| | CATCAAGCCAGCCATTGAGAACTGGGGCAGTGACTTCCTGTGTACAGAGTGGAAGGCT | | |
| | TCCAATAGTGTTCCAACCTCTGTCCACCAGCTCCGACCAGCAGACATCAAAGTGGTGG | | |
| | CCGCCCTGGGTGACTCTCTGACTACAGCAGTGGGAGCTCGACCAAACAACTCCAGTGA | | |
| | CCTACCCACATCTTGGAGGGGACTCTCTTGGAGCATTGGAGGGGATGGGAACTTGGAG | | |
| | ACTCACACCACACTGCCCAGTATTCTGAAGAAGTTCAACCCTTACCTCCTTGGCTTCT | | |
| | CTACCAGCACCTGGGAGGGGACAGCAGGACTAAATGTGGCAGCGGAAGGGGCCAGAGC | | |
| | TAGGAGGGACATGCCAGCCCAGGCCTGGGACCTGGTAGAGCGAATGAAAAACAGCCCC | | |
| | ATACACTTTCAGGAAGACTGGAAGATAATAACCCTGTTTATAGGCGGCAATGACCTCT | | |
| | GTGATTTCTGCAATGATCTGGTAGGTGAATATGTTCAGCACATCCAACAGGCCCTGGA | | |
| | CATCCTCTCTGAGGAGCTCCCAAGGGCTTTCGTCAACGTGGTGGAGGTCATGGAGCTG | | |
| | GCTAGCCTGTACCAGGGCCAAGGCGGGAAATGTGCCATGCTGGCAGCTCAGAACAACT | | |
| | GCACTTGCCTCAGACACTCGCAAAGCTCCCTGGAGAAGCAAGAACTGAAGAAAGTGAA | | |
| | CTGGAACCTCCAGCATGGCATCTCCAGTTTCTCCTACTGGCACCAATACACACAGCGT | | |
| | GAGGACTTTGCGGTTGTGGTGCAGCCTTTCTTCCAAAACACACTCACCCCACTGAACA | | |
| | GAGGGGACACTGACCTCACCTTCTTCTCCGAGGACTGTTTTCACTTCTCAGACCGCGG | | |
| | GCATGCCGAGATGGCCATCGCACTCTGGAACAACATGCTGGAACCAGTGGGCCGCAAG | | |
| | ACTACCTCCAACAACTTCACCCACAGCCGAGCCAAACTCAAGTGCCCCTCTCCTGTGA | | |
| | GTCCTTACCTCTACACCCTGCGGAACAGCCGATTGCTCCCAGACCAGGCTGAAGAAGC | | |
| | CCCCGAGGTGCTCTACTGGGCTGTCCCAGTGGCAGCGGGAGTCGGCCTTGTGGTGGGC | | |
| | ATCATCGGGACAGTGGTCTGGAGGTGCAGGAGAGGTGGCCGGAGGGAAGATCCTCCAA | | |
| | TGAGCCTGCGCACTGTGGCCCTCTAGGCCCGGGG | | |
| | ORF Start: ATG at 1 | | ORF Stop: TAG at 4258 |
| | SEQ ID NO:82 | 1419 aa | MW at 158435.1 kD |
| NOV24a, | MTWDTALWTSVFLIGLLPTLGFANCILQTSGKMCTLRGRYPQPPQPPLCLSPLVHQLR | | |
| CG57399-01 Protein | | | |
| Sequence | PADIKVVAALGNDETFQESGAGQLSEPDPRQWSWPQACLPGVKKEMQDVVGERTPSRR | | |
| | RSLRRREALVPAAGKESLCRQDIFISLLEIIKHFPPSPQDINLEKDWKLVTLFIGVND | | |
| | LCHYCPLVQGPVIDLGGMDTLHSLQLPRAFVNVVEVMELASLYQGQGGKCAMLAAQEA | | |
| | WNSLLASSRYSEQESFTVVFQPFFYETTPSDPRLQDSTTLAWHLWNRMMEPAGEKDEP | | |
| | LSVKHGRPMKCPSQESPYLFSYRNSNYLTRLQKPQDKLVREGAEIRCPDKDPSDTVPT | | |
| | SVHRLKPADINVIGALGDSLTAGNGAGSTPGNVLDVLTQYRGLSWSVGGDENIGTVTT | | |
| | LADILREFNPSLKGFSVGTGKETSPNAFLNQAVAGGRAEQARRLVDLMKNDTRIHFQE | | |
| | DWKIITLFIGGNDLCDETNDLVHYSPQNFTDNTGKALDILHAESQVPRAFVNLVTVLE | | |
| | IVNLRELYQEKKVYCPRMILRSLCPCVLKFDDNSTELATLIEFNKKFQEKTHQLIESG | | |
| | RYDTREDFTVVVQPFFENVDMPKTQEGLPDNSFFAPDCFHFSSKSHSRAASALWNNML | | |
| | EPVGQKTTRHKFENKINITCPSQVQPFLRTYKNSMQGHGTWLPCRDRAPSALHPTSVH | | |
| | ALRPADIQVVAALGDSLTAGNGIGSKPDDLPDVTTQYRGLSYSAGGDGSLENVTTLPS | | |
| | SILREFNRNLTGYAVGTGDANDTNAFLNQAVPGAKARDLMSQVQTLMQKMKDDHRVNF | | |
| | HEDWKVITVLIGGSDLCDYCTDSNLYDAANFVHHLRNALDVLHREVPRVLVNLVDFLN | | |
| | PTIMRQVFLGNPDKCPVQQASVLCNCVLTLRENSQELARLEAFSRAYQSSMRELVGSG | | |
| | RYDTQEDFSVVLQPFFQNIQLPVLQDGLPDTSFFAPDCIHPNQKFHSQLARALWTNML | | |
| | EPLGSKTETLDLRAEMPITCPTQNEPFLRTPRNSNYTYPIKPAIENWGSDFLCTEWKA | | |
| | SNSVPTSVHQLRPADIKVVAALGDSLTTAVGARPNNSSDLPTSWRGLSWSIGGDGNLE | | |
| | THTTLPSILKKFNPYLLGFSTSTWEGTAGLNVAAEGARARRDMPAQAWDLVERMKNSP | | |
| | IHFQEDWKIITLFIGGNDLCDFCNDLVGEYVQHIQQALDILSEELPRAFVNVVEVMEL | | |
| | ASLYQGQGGKCAMLAAQNNCTCLRHSQSSLEKQELKKVNWNLQHGISSFSYWHQYTQR | | |
| | EDFAVVVQPFFQNTLTPLNRGDTDLTFFSEDCFHFSDRGHAEMAIALWNNMLEPVGRK | | |
| | TTSNNFTHSRAKLKCPSPVSPYLYTLRNSRLLPDQAEEAPEVLYWAVPVAAGVGLVVG | | |
| | IIGTVVWRCRRGGRREDPPMSLRTVAL | | |
| | SEQ ID NO:83 | | 1624 bp |
| NOV24b, | GCCGGCTGACATCAATGTAATTGGAGCCCTGGGTGACTCTCTCACGGCAGGCAATGGG | | |
| CG57399-02 DNA Sequence | | | |
| | GCCGGGTCCACACCTGGGAACGTCTTGGACGTCTTGACTCAGTACCGAGGCCTGTCCT | | |
| | GGAGCGTCGGCGGAGATGAGAACATCGGCACCGTTACCACCCTGGCGAACATCCTCCG | | |
| | GGAATTCAACCCTTCCCTGAAGGGCTTCTCTGTTGGCACTGGGAAAGAAACCAGTCCT | | |
| | AATGCCTTCTTAAACCAGGCTGTGGCAGGAGGCCGAGCTGAGGATCTACCTGTCCAGG | | |
| | CCAGGAGGCTGGTGGACCTGATGAAGAATGACACGAGGATACACTTTCAGGAAGACTG | | |
| | GAAGATAATAACCCTGTTTATAGGCGGCAATGACCTCTGTGATTTCTGCAATGATCTG | | |
| | GTCCACTATTCCCCCCAGAACTTCACAGACAACATTGGAAAGGCCCTGGACATCCTCC | | |
| | ATGCTGAGGTTCCTCGGGCATTTGTGAACCTGGTGACGGTGCTTGAGATCGTCAACCT | | |
| | GAGGGAGCTGTACCAGGAGAAAAAAGTCTACTGCCCAAGGATGATCCTCAGGTCTCTG | | |
| | TGTCCCTGTGTCCTGAAGTTTGATGATAACTCAACAGAACTTGCTACCCTCATCGAAT | | |
| | TCAACAAGAAGTTTCAGGAGAAGACCCACCAACTGATTGAGAGTGGGCGATATGACAC | | |
| | AAGGGAAGATTTTACTGTGGTTGTGCAGCCGTTCTTTGAAAACGTGGACATGCCAAAG | | |
| | ACCTCGGAAGGATTGCCTGACAACTCTTTCTTCGCTCCTGACTGTTTCCACTTCAGCA | | |
| | GCAAGTCTCACTCCCGAGCAGCCAGTGCTCTCTGGAACAATATGCTGGAGCCTGTTGG | | |
| | CCAGAAGACGACTCGTCATAAGTTTGAAAACAAGATCAATATCACATGTCCGAACCAG | | |
| | GTCCAGCCGTTTCTGAGGACCTACAAGAACAGCATGCAGGGTCATGGGACCTGGCTGC | | |
| | CATGCAGGGACAGAGCCCCTTCTGCCTTGCACCCTACCTCAGTGCATGCCCTGAGACC | | |
| | TGCAGACATCCAAGTTGTGGCTGCTCTGGGGGATTCTCTGACCGCTGGCAATGGAATT | | |
| | GGCTCCAAACCAGACGACCTCCCCGATGTCACCACACAGTATCGGGGACTGTCATACA | | |
| | GAGAAAGTAAACCAGGGTTCTTATCAGACTCCTGGGTCAGCAAATCCAACAGGAAATG | | |
| | CACCAGAAAAGCACCAAATCCCTGAATCTTCACCTCCCCGCTTGCATGTATACGTGTA | | |
| | CACGTGGTGTTCCTACGTCTCTGTTTACTGTCTTTATGTGTTTATTCATGTTGTCTTG | | |
| | TAGTCACACAGCTGCCTTTACATATATGTACACATCTGCACAGAAAACCTCTGAAACC | | |
| | CATCGCACACTTCGAGAGGCCATAACCAAGACACAATCACAATCAGCCATGTCTTGAA | | |
| | AGATTAGCAATTCGACAAGAGGAAAGGGTGAGAAAGGGCATCCCGAACACGGAAGTGG | | |
| | AGAAGCTCAGGGTGTGTCAGGCGAGCGGTTGCGTGTAGATATTCTCAAGTTTCTTTCT | | |
| | CTCCTAATAAAGTTCTCATTCCTGTAGGCTTCAAAGTAAGTGGCGAGTAGCTCAGAAT | | |
| | ORF Start: ATG at 311 | | ORF Stop: TGA at 1241 |
| | SEQ ID NO:84 | 310 aa | MW at 35240.6 kD |
| NOV24b, | MKNDTRIHFQEDWKIITLFIGGNDLCHFCNDLVHYSPQNFTDNIGKALDILHAEVPRA | | |
| CG57399-02 Protein | | | |
| Sequence | FVNLVTVLEIVNLRELYQEKKVYCPRMILRSLCPCVLKFDDNSTELATLIEFNKKFQE | | |
| | KTHQLIESGRYDTREDPTVVVQPFFENVDMPKTSEGLPDNSFFAPDCFHFSSKSHSRA | | |
| | ASALWNNMLEPVGQKTTRHKFENKINITCPNQVQPFLRTYKNSMQGHGTWLPCRDRAP | | |
| | SALHPTSVHALRPADIQVVAALGDSLTAGNGIGSKPDDLPDVTTQYRGLSYRESKPGF | | |
| | LSDSWVSKSNRKCTRKAPNP | | |
| | SEQ ID NO:85 | 4425 bp | |
| NOV24c, | CTGGAGCATTCTGGCATGGGGCTGCGGCCAGGCATTTTCCTCCTGGAGCTGCTGCTGC | | |
| CG57399-03 DNA Sequence | | | |
| | TTCTGGGGCAAGGTACCCCTCAGATCCATACCTCTCCTAGAAAGAGTACATTGGAAGG | | |
| | GCAGCTATGGCCAGAGACAGTTCACTCTCTGAAGCCTTCTGATATTAAATTTGTGGCA | | |
| | GCCATTGGCAATCTGGAAATTGTGCCAGACCCAGGGACGGGCGATCTGGAGAAGCAAG | | |
| | ACGAAAGGCCACAGCAGGTGTGCATGGGAGTGATGACAGTCCTTTCAGACATCATCAG | | |
| | ATATTTCAGTCCTTCTGTTCCAATGCCTGTGTGCCACACTGGAAAGAGAGTCATACCC | | |
| | CACGATGGTGCTGAGGACTTGTGGATTCAGGCTCAAGAACTGGTGAGAAACATGAAAG | | |
| | AGAACCAACTTGACTTTCAATTTGACTGGAAGCTCATCAATGTGTTCTTCAGTAATGC | | |
| | AAGCCAGTGTTACCTGTGCCCCTCTGCTCAACAGAATGCGCTTGCGGCGGGCGGCGTG | | |
| | GATGAGCTGATGGGGGTGCTGGACTACCTGCAGCAGGAGGTGCCCAGAGCATTTGTAA | | |
| | ACCTGGTGGACCTCTCTGAGGTTGCAGAGGTCTCTCGTCAGTATCACGGCACTTGGCT | | |
| | CAGCCCTGCACCAGAGCCCTGTAATTGCTCAGAGGAGACCACCCGGCTGGCCAAGGTG | | |
| | GTGATGCAGTGGTCTTATCAGGAAGCCTGGAACAGCCTCCTGGCCTCCAGCAGGTACA | | |
| | GTGAGCAGGAGTCCTTCACCGTGGTTTTCCAGCCTTTCTTCTATGAGACCACCCCATC | | |
| | TGACCCCCGACTCCAGGATTCTACCACGCTGGCCTGGCATCTCTGGAATAGGATGATG | | |
| | GAGCCAGCAGGAGAGAAGATGAGCCATTGAGTGTAAAACACGGGAGGCCAAATGAAGT | | |
| | GTCCCTCTCAGGAGAGCCCCTATCTGTTCAGCTACAGAAACAGCAACTACCTGACCAG | | |
| | ACTGCAGAAACCCCAAGACAAGCTTGAGGTAAGAGAAGGAGCGGAAATCAGATGTCCT | | |
| | GACAAAGACCCCTCCGATACGGTTCCCACCTCAGTTCATAGGCTGAAGCCGGCTGACA | | |
| | TCAACGTAATTGGAGCCCTGGGTGACTCTCTCACGGCAGGCAATGGGGCCGGGTCCAC | | |
| | ACCTGGGAACCTCTTGGACGTCTTGACTCAGTACCGAGGCCTGTCCTGGAGCGTCGGC | | |
| | GGAGATGAGAACATCGGCACCGTTACCACCCTGGCGGACATCCTCCGGGAATTCAACC | | |
| | CTTCCCTGAAGGGCTTCTCTGTTGGCACTGGGAAAGAAACCAGTCCTAATGCCTTCTT | | |
| | AAACCAGGCTGTGGCAGGAGGCCGAGCTGAGCAGGCCAGGAGGCTGGTGGACCTGATG | | |
| | AAGAATGACACGAGGATACACTTTCAGGAAGACTCCAACATAATAACCCTGTTTATAG | | |
| | GCGGCAATGACCTCTGTGATTTCTGCAATGATCTGGTACACTATTCTCCCCAGAACTT | | |
| | CACAGACAACATTGGAAAGGCCCTGGACATCCTCCATGCTGAGGTTCCTCGGGCATTT | | |
| | GTGAACCTGGTGACGGTGCTTGAGATCGTCAACCTGAGGGAGCTGTACCAGGACAAAA | | |
| | AAGTCTACTGCCCAAGGATGATCCTCAGGTCACTGTGTCCCTGTGTCCTGAAGTTTGA | | |
| | TGATAACTCAACAGAACTTGCTACCCTCATCGAATTCAACAACAAGTTTCAGGAGAAG | | |
| | ACCCACCAACTGATTGAGAGTGGGCGATATGACACAAGGGAAGATTTTACTGTGGTTG | | |
| | TGCAGCCGTTCTTTGAAAACGTGCACATGCCAAAGACCCAGGAAGGATTGCCTGACAA | | |
| | CTCTTTCTTCGCTCCTGACTGTTTCCACTTCAGCAGCAAGTCTCACTCCCGAGCAGCC | | |
| | AGTGCTCTCTGGAACAATATGCTGGAGCCTGTTOCCCAGAAGACGACTCGTCATAAGT | | |
| | TTGAAAACAAGATCAATATCACATGTCCGAACCACGTAGAGTGGCCGTTTCTGAGGAC | | |
| | CTACAAGAACAGCATGCAGGGTCATGGGACCTGGCTGCCATGCAGGGACAGAGCCCCT | | |
| | TCTGCCTTGCACCCTACCTCAGTCCATGCCCTGAGACCTGCAGACATCCAAGTTGTGG | | |
| | CTGCTCTGGGGGATTCTCTGACCGCTGGCAATGGAATTGGCTCCAAACCAGACGACCT | | |
| | CCCCGATGTCACCACACAGTATCGGGGACTGTCATACAGTGCAGGAGGGGACGGCTCC | | |
| | CTGGAGAATGTGACCACCTTACCTGATATCCTTCGGGAGTTTAACAGAAACCTCACAG | | |
| | GCTACGCCGTGGGCACGGGTGATCCCAATGACACGAATGCATTCCTCAATCAAGCTGT | | |
| | TCCCGGAGCAAAGGCTAGGGATCTTATGAGCCAAGTCCAAACTCTGATGCACAAGATG | | |
| | AAAGATGATCATAGAGTAAATTTCCATGAAGACTGGAAGGTCATCACAGTGCTGATCG | | |
| | GAGGCAGCGATTTATGTGACTACTGCACAGATTCGAATCTGTATTCTGCAGCCAACTT | | |
| | TGTTCACCATCTCCGCAATGCCTTGGACGTCCTGCATAGAGAGGTGCCCAGAGTCCTG | | |
| | GTCAACCTCGTCGACTTCCTGAACCCCACTATCATGCGGCAGGTGTTCCTGGGAAACC | | |
| | CAGACAAGTGCCCAGTGCAGCAGGCCAGCGTTTTGTGTAACTGCGTTCTGACCCTGCG | | |
| | GGAGAACTCCCAAGAGCTAGCCAGGCTGGAGGCCTTCAGCCGACCCTACCAGAGCAGC | | |
| | ATGCGCGAGCTGGTGGGGTCAGGCCGCTATGACACGCAGGAGGACTTCTCTGTGGTGC | | |
| | TGCAGCCCTTCTTCCAGAACATCCAGCTCCCTGTCCTGCAGGATGGGCTCCCAGATAC | | |
| | GTCCTTCTTTGCCCCAGACTGCATCCACCCAAATCAGAAATTCCACTCCCAGCTGGCC | | |
| | AGAGCCCTTTGGACCAATATGCTTGAACCACTTGGAAGCAAAACAGAGACCCTGGACC | | |
| | TGAGAGCAGAGATGCCCATCACCTGTCCCACTCAGAATGAGCCCTTCCTGAGAACCCC | | |
| | TCGGAATAGTAACTACACGTACCCCATCAAGCCAGCCATTGAGAACTGGGGCAGTGAC | | |
| | TTCCTGTGTACAGAGTGGAAGGCTTCCAATAGTGTTCCAACCTCTGTCCACCAGCTCC | | |
| | CACCAGCAGACATCAAAGTGGTGGCCGCCCTGGGTGACTCTCTGACTGTGGCAGTGGG | | |
| | AGCTCGACCAAACAACTCCAGTGACCTACCCACATCTTGGAGGGGACTCTCTTGGAGC | | |
| | ATTGGAGGGGATGGGAACTTGGAGACTCACACCACACTGCCCGACATTCTGAAGAAGT | | |
| | TCAACCCTTACCTCCTTGGCTTCTCTACCAGCACCTGGGAGGGGACAGCAGGACTAAA | | |
| | TGTGGCAGCGGAAGGGGCCAGAGCTAGGGACATGCCAGCCCAGGCCTGGGACCTGGTA | | |
| | GAGCGAATGAAAAACAGCCCCCAGGACATCAACCTGGAGAAAGACTGGAAGCTGGTCA | | |
| | CACTCTTCATTGGGGTCAACGACTTGTGTCATTACTGTGAGAATCCGGTAGGCGAATA | | |
| | TGTTCAGCACATCCAACAGGCCCTGGACATCCTCTCTGAGGAGCTCCCAAGGGCTTTC | | |
| | GTCAACGTGGTGGAGGTCATGGAGCTGGCTAGCCTGTACCAGGGCCAAGGCGGGAAAT | | |
| | GTGCCATGCTGGCAGCTCAGAACAACTGCACTTGCCTCAGACACTCGCAAAGCTCCCT | | |
| | GGAGAAGCAAGAACTGAAGAAAGTGAACTGGAACCTCCAGCATGGCATCTCCAGTTTC | | |
| | TCCTACTGGCACCAATACACACAGCGTGAGGACTTTGCGGTTGTGGTGCAGCCTTTCT | | |
| | TCCAAAACACACTCACCCCACTGAACAGAGGGGACACTGACCTCACCTTCTTCTCCGA | | |
| | GGACTGTTTTCACTTCTCAGACCGCGGGCATGCCGAGATGGCCATCGCACTCTGGAAC | | |
| | AACATGCTGGAACCAGTGGGCCGCAAGACTACCTCCAACAACTTCACCCACAGCCGAG | | |
| | CCAAACTCAAGTGCCCCTCTCCTGAGAGCCCTTACCTCTACACCCTGCGGAACAGCCG | | |
| | ATTGCTCCCAGACCAGGCTGAAGAAGCCCCCGAGGTGCTCTACTGGGCTGTCCCAGTG | | |
| | GCAGCGGGAGTCGGCCTTGTGGTGGGCATCATCGGGACAGTGGTCTGGAGGTGCAGGA | | |
| | GAGGTGGCCGGAGGGAAGATCCTCCAATGAGCCTGCGCACTGTGGCCCTCTAGGCCCG | | |
| | GGGGTGGGTCCTCACCCTAAACTCCCTATAGCCACTCTCTTCACCGCCCTCTGCCCCA | | |
| | GCCACTCCCGGCCACCAGGACATGCTTCAATGCCTGGTGCCATAGGAAGCCCAGGGGA | | |
| | CAGTCACAACTTCTTGG | | |
| | ORF Start ATG at 16 | | ORF Stop: TAG at 4285 |
| | SEQ ID NO:86 | 1423 aa | MW at 159352.7 kD |

| | |
|---|---|
| NOV24c, | MGLRPGIFLLELLLLLGQGTPQIHTSPRKSTLEGQLWPETVHSLKPSKIKFVAAIGNL |
| CG57399-03 Protein | |
| Sequence | EIVPDPGTGDLEKQDERPQQVCMGVMTVLSDIIRYFSPSVPMPVCHTGKRVIPHDGAE |
| | DLWIQAQELVRNMKENQLDFQFDWKLINVFFSNASQCYLCPSAQQNGLAAGGVDELMG |
| | VLDYLQQEVPRAFVNLVDLSEVAEVSRQYHGTWLSPAPEPCNCSEETTRLAKVVMQWS |
| | YQEAWNSLLASSRYSEQESFTVVFQPFFYETTPSDPRLQDSTTLAWHLWNRMMEPAGE |
| | KDEPLSVKHGRPMKCPSQESPYLFSYRNSNYLTRLQKPQDKLEVREGAEIRCPDKDPS |
| | DTVPTSVHRLKPADINVIGALGDSLTAGNGAGSTPGNVLDVLTQYRGLSWSVGGDENI |
| | GTVTTLADILREFNPSLKGFSVGTGKETSPNAFLNQAVAGGRAEQARRLVDLMKNDTR |
| | IHFQEDWKIITLFIGGNDLCDFCNDLVHYSPQNFTDNIGKALDILHAEVPRAFVNIVT |
| | VLEIVNLRELYQEKKVYCPRMILRSLCPCVLKFDDNSTELATLIEFWKKFQEKTHQLI |
| | ESGRYDTREDFTVVVQPFFENVDMPKTQEGLPDNSFFAPDCFHFSSKSHSRAASALWN |
| | NMLEPVGQKTTRHKFENKINITCPNQVEWPFLRTYKNSMQGHGTWLPCRDRAPSAIHP |
| | TSVHALRPADIQVVAALGDSLTAGNGIGSKPDDLPDVTTQYRGLSYSAGGDGSLENVT |
| | TLPDILREFNRNLTGYAVGTGDANDTNAFLNQAVPGAKARDLMSQVQTLMQKMKDDHR |
| | VNFHEDWKVITVLIGGSDLCDYCTDSNLYSAANFVHHLRNALDVLHREVPRVLVNLVD |
| | FLNPTIMRQVFLGNPDKCPVQQASVLCNCVLTLRENSQELARLEAFSRAYQSSMRELV |
| | GSGRYDTQEDFSVVLQPFFQNIQLPVLQDGLPDTSFFAPDCIHPNQKFHSQLARALWT |
| | NNLEPLGSKTETLDLRAEMPITCPTQNEPFLRTPRNSNYTYPIKPAIENWGSDFLCTE |
| | WKASNSVPTSVHQLRPADIKVVAALGDSLTVAVGARPNNSSDLPTSWRGLSWSIGGDG |
| | NLETHTTLPDILKKPNPYLLGPSTSTWEGTAGLNVAAEGARARDMPAQAWDLVERMKN |
| | SPQDINLEKDWKLVTLFIGVNDLCHYCENPVGEYVQHIQQALDILSEELPRAFVNVVE |
| | VMELASLYQGQGGKCAMLAAQNNCTCLRHSQSSLEKQELKKVNWNLQHGISSFSYWHQ |
| | YTQREDFAVVVQPFFQNTLTPLNRGDTDLTFFSEDCFHFSDRGHAEMAIALWNNMLEP |
| | VGRKTTSNNFTHSRAKLKCPSPESPYLYTLRNSRLLPDQAEEAPEVLYWAVPVAAGVG |
| | LVVGIIGTVVWRCRRGGRREDPPMSLRTVAL |

Sequence comparison of the above protein sequences yields the following sequence relationships shown in Table 24B.

TABLE 24B
Comparison of NOV24a against NOV24b through NOV24c.

| Protein | NOV24a Residues/ | Identities/Similarities |
|---|---|---|
| Sequence | Match Residues | for the Matched Region |
| NOV24b | 454 . . . 748 | 283/295 (95%) |
| | 1 . . . 293 | 285/295 (95%) |
| NOV24c | 27 . . . 1419 | 1211/1426 (84%) |
| | 23 . . . 1423 | 1261/1426 (87%) |

Further analysis of the NOV24a protein yielded the following properties shown in Table 24C.

TABLE 24C
Protein Sequence Properties NOV24a

| | |
|---|---|
| PSort | 0.6850 probability located in endoplasmic reticulum |
| analysis: | (membrane); 0.6400 probability located in plasma membrane; |
| | 0.4600 probability located in Golgi body; 0.1080 probability |
| | located in nucleus |
| SignalP | Likely cleavage site between residues 24 and 25 |
| analysis: | |

A search of the NOV24a protein against the Geneseq database, a proprietary database that contains sequences published in patents and patent publication, yielded several homologous proteins shown in Table 24D.

TABLE 24D
Geneseq Results for NOV24a

| | | | Identities/ | |
|---|---|---|---|---|
| | | NOV24a | Similarities | |
| | Protein/ | Residues/ | for the | |
| Geneseq | Organism/Length | Match | Matched | Expect |
| Identifier | [Patent #, Date] | Residues | Region | Value |
| AAW30751 | Rat phospho- | 50 . . . 1403 | 911/1404 | 0.0 |
| | lipase-B/lipase— | | (64%) | |
| | Rattus rattus | 60 . . . 1447 | 1077/1404 | |
| | 1450 aa. | | (75%) | |
| | [JP09248190-A, | | | |
| | Sep. 22, 1997] | | | |
| ABB11053 | Human phospho- | 985 . . . 1203 | 205/224 | e−117 |
| | lipase B Homo | | (91%) | |
| | sapiens, 267 aa. | 45 . . . 267 | 213/224 | |
| | [WO200157188- | | (94%) | |
| | A2, Aug. 9, | | | |
| | 2001] | | | |
| AAM25824 | Human protein | 985 . . . 1203 | 205/224 | e−117 |
| | sequence SEQ ID | | (91%) | |
| | NO:1339—Homo | 45 . . . 267 | 213/224 | |
| | sapiens, 267 aa. | | (94%) | |
| | [WO200153455- | | | |
| | A2, Jul. 26, 2001] | | | |
| AAM95420 | Human | 979 . . . 1106 | 110/130 | 3e−56 |
| | reproductive | | (84%) | |
| | system related | 4 . . . 133 | 117/130 | |
| | antigen SEQ ID | | (89%) | |
| | NO:4078—Homo | | | |
| | sapiens, 148 aa. | | | |
| | [WO200155320- | | | |
| | A2, Aug. 2, | | | |
| | 2001] | | | |
| ABB11237 | Human phospho- | 393 . . . 478 | 84/90 | 3e−40 |
| | lipase homologue, | | (93%) | |
| | SEQ ID NO: | 43 . . . 132 | 86/90 | |
| | 1607—Homo | | (95%) | |
| | sapiens, 132 aa. | | | |
| | [WO200157188- | | | |
| | A2, Aug. 9, | | | |
| | 2001] | | | |

In a BLAST search of public sequence databases, the NOV24a protein was found to have homology to the proteins shown in the BLASTP data in Table 24E.

TABLE 24E
Public BLASTP Results for NOV24a

| | | NOV24a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q05017 | Phospholipase | 6 . . . 1416 | 1042/1466 | 0.0 |
| | ADRAB-B | | (71%) | |
| | precursor | 2 . . . 1456 | 1179/1466 | |
| | (EC 3.1.-.-)— | | (80%) | |
| | Oryctolagus | | | |
| | cuniculus | | | |
| | (Rabbit), 1458 aa. | | | |
| O70320 | PHOSPHO- | 7 . . . 1414 | 965/1474 | 0.0 |
| | LIPASE B— | | (65%) | |
| | Cavia porcellus | 3 . . . 1458 | 1135/1474 | |
| | (Guinea pig), | | (76%) | |
| | 1463 aa. | | | |
| O54728 | PHOSPHO- | 50 . . . 1403 | 911/1404 | 0.0 |
| | LIPASE B— | | (64%) | |
| | Rattus norvegicus | 60 . . . 1447 | 1077/1404 | |
| | (Rat), 1450 aa. | | (75%) | |
| Q96DP9 | CDNA FLJ30866 | 454 . . . 714 | 257/261 | e−151 |
| | FIS, CLONE | | (98%) | |
| | FEBRA2004110, | 1 . . . 259 | 258/261 | |
| | HIGHLY | | (98%) | |
| | SIMILAR TO | | | |
| | PHOSPHO- | | | |
| | LIPASE | | | |
| | ADRAB-B | | | |
| | PRECURSOR | | | |
| | (EC 3.1.-.-)— | | | |
| | Homo sapiens | | | |
| | (Human), 270 aa. | | | |
| Q9N2Z4 | HYPO- | 343 . . . 673 | 130/343 | 1e−59 |
| | THETICAL | | (37%) | |
| | 41.4 KDA | 37 . . . 369 | 202/343 | |
| | PROTEIN— | | (57%) | |
| | Caenorhabditis | | | |
| | elegans, 377 aa. | | | |

PFam analysis predicts that the NOV24a protein contains the domains shown in the Table 24F.

TABLE 24F
Domain Analysis of NOV24a

| | | Identities/ | |
|---|---|---|---|
| | NOV24a | Similarities for | Expect |
| Pfam Domain | Match Region | the Matched Region | Value |
| Lipase_GDSL: domain | 360 . . . 484 | 54/147 (37%) | 4.8e−42 |
| 1 of 3 | | 116/147 (79%) | |
| Lipase_GDSL: domain | 705 . . . 834 | 57/147 (39%) | 4.5e−44 |
| 2 of 3 | | 116/147 (79%) | |
| SecA_protein: domain | 834 . . . 851 | 10/20 (50%) | 4.9 |
| 1 of 1 | | 17/20 (85%) | |
| Vitellogenin_N: domain | 1107 . . . 1124 | 8/18 (44%) | 3.8 |
| 1 of 1 | | 17/18 (94%) | |
| Lipase_GDSL: domain | 1062 . . . 1185 | 48/147 (33%) | 6.3e−37 |
| 3 of 3 | | 114/147 (78%) | |

### Example 25

The NOV25 clone was analyzed, and the nucleotide and predicted polypeptide sequences are shown in Table 25A.

TABLE 25A
NOV25 Sequence Analysis

| | SEQ ID NO:87 | 1348 bp | |
|---|---|---|---|
| NOV25a, | CTGGGTCGCCCCTGTTCTACCCAGATTGGGATGGCAGCGACGCTGATCCTGGAGCCCG | | |
| CG59311-01 DNA Sequence | | | |
| | CGGGCCGCTGCTGCTGGGACGAGCCGCTGCGCATCGCAGTGCGCGGCCTGGCCCCGGA | | |
| | GCAGCCAGTCACGCTGCGCACGTCCCTGCGCGACGAAGAGGGCGCGCTCTTCCGGGCC | | |
| | CACGCGCGCTACCGTGCCGACGCCCGCGACGAGCTGGACCTGGAGCGCGCGCCCGCGC | | |
| | TGGGAGGCAGCTTCGCGGGGCTCCAGCCCATGGGGCTGCTGTGGGCGTTGGAGCCCGA | | |
| | GAAAGCCTTGGTGCGGCTGGTGAAGCGCGACGTGCGGACGCCCTTCGCCGTGGAGCTG | | |
| | GAAGTGCTGGACGGCCACGACACCGAGCCCGGGCGGCTGCTGTGCCTGGCGCAGAACA | | |
| | AGCGCGACTTTCTCCGGCCGGGGGTGCGGCGCGAGCCGGTGCGCGCGGGCCCGGTGCG | | |
| | CGCCGCGCTCTTCCTGCCGCCGGATAGGGGGCCCTTTCCTGGGATCATTGATCTGTTT | | |
| | GGGAGCAGCAGGGGCCTTTGTGAATACAGGGCCAGCCTCCTGGCCGGACATGGTTTTG | | |
| | CTGTGCTTGCCCTGGCTTATTTCAGATTTGAAGACCTCCCCGAAGATCTGAATGATGT | | |
| | ACATCTGGAGTACTTTGAAGAAGCCGTGGACTTTATGCTGCAGCATCCAAAGGTGAAA | | |
| | GGTCCTAGTATTGCGCTTCTTGGATTTTCCAAAGGAGGTGACCTGTGTCTCTCAATGG | | |
| | CTTCTTTCTTGAAGGGCATCACAGCCACTGTACTTATCAATGCCTGTGTAGCCAACAC | | |
| | AGTAGCTCCTCTACATTACAAGGATATGATTATTCCTAAACTTGTCGATGATCTAGGA | | |
| | AAAGTAAAAATCACTAAGTCAGGATTTCTCACTTTTATGGACACTTGGAGCAATCCAC | | |
| | TGGAGGAACACAATCACCAAAGTCTTGTTCCATTGGAAAAGGCGCAGGTGCCCTTCTT | | |
| | GTTTATTGTTGGCATGGATGATCAAAGCTGGAAGAGTGAATTCTATGCTCAGATAGCC | | |
| | TCTGAAAGGCTACAAGCTCATGGGAAAGAAAGACCCCAGATAATCTGTTACCCAGAAA | | |
| | CTGGTCACTGTATTGACCCACCTTATTTTCCTCCTTCTAGAGCTTCTGTGCACGCTGT | | |
| | TTTGGGTGAGGCAATATTCTATGGAGGTGAGCCAAAGGCTCACTCAAAGGCACAGGTA | | |
| | GATGCCTGGCAGCAAATTCAAACTTTCTTCCATAAACATCTCAATGGTAAAAAATCTG | | |
| | TCAAGCACAGCAAAATATAACATTGTAGCCACAGACCAGATACCATTAATAAAAATCC | | |
| | TATTCATACAACTT | | |
| | ORF Start: ATG at 31 | | ORF Stop: TAA at 1294 |
| | SEQ ID NO:88 | 421 aa | MW at 46815.4 kD |
| NOV25a, | MAATLILEPAGRCCWDEPLRIAVRGLAPEQPVTLRTSLRDEEGALFRAHARYRADARD | | |
| CG59311-01 Protein Sequnce | | | |
| | ELDLERAPALGGSFAGLQPMGLLWALEPEKALVRLVKRDVRTPFAVELEVLDGHDTEP | | |
| | GRLLCLAQNKRDFLRPGVRREPVRAGPVRAALFLPPDRGPFPFIIDLFGSSRGLCEYR | | |
| | ASLLAGHGFAVLALAYFRFEDLPEDLNDVHLEYFEEAVDFMLQHPKVKGPSIALLGFS | | |
| | KGGDLCLSMASFLKGITATVLINACVANTVAPLHYKDMIIPKLVDDLGKVKITKSGFL | | |
| | TFMDTWSNPLEEHNHQSLVPLEKAQVPFLFIVGMDDQSWKSEFYAQIASERLQAHGKE | | |
| | RPQIICYPETGHCIDPPYFPPSRASVHAVLGEAIFYGGEPKAHSKAQVDAWQQIQTFF | | |
| | HKHLNGKKSVKHSKI | | |
| | SEQ ID NO:89 | 1021 bp | |
| NOV25b, | AGATTGGGATGGCAGCGACGCTGATCCTGGAGCCCGCGGGCCGCTGCTGCTGGGACGA | | |
| CG59311-01 DNA Sequence | | | |
| | GCCGCTGCGCATCGCAGTGCGCGGCCTGGCCCCGGAGCAGCCAGTCACGCTGCGCACG | | |
| | TCCCTGCGCGACGAAGAGGGCGCGCTCTTCCGGGCCCACGCGCGCTACCGTGCCGACG | | |
| | CCTCTAATCCCGGCACTTTGGGGGGCCAAGGCAGGGGGCCCTTTCCTGGGATCATTGA | | |
| | TCTGTTTGGGAGCAGCAGGGGCCTTTGTGAATACAGGGCCAGCCTCCTGGCCGGACAT | | |
| | GGTTTTGCTGTGCTTGCCCTGGCTTATTTCAGATTTGAAGACCTCCCCGAAGATCTGA | | |
| | ATGATGTACATCTGGAGTACTTTGAAGAAGCCGTGGACTTTATGCTGCAGCATCCAAA | | |
| | GGTGAAAGGTCCTAGTATTGCGCTTCTTGGATTTTCCAAAGGAGGTGACCTGTGTCTC | | |
| | TCAATGGCTTCTTTCTTGAAGGGCATCACAGCCACTGTACTTATCAATGCCTGTGTAG | | |
| | CCAACACAGTAGCTCCTCTACATTACAAGGATATGATTATTCCTAAACTTGTCGATGA | | |
| | TCTAGGAAAAGTAAAAATCACTAAGTCAGGATTTCTCACTTTTATGGACACTTGGAGC | | |
| | AATCCACTGGAGGAACACAATCACCAAAGTCTTGTTCCATTGGAAAAGGCGCAGGTGC | | |
| | CCTTCTTGTTTATTGTTGGCATGGATGATCAAAGCTGGAAGAGTGAATTCTATGCTCA | | |
| | GATAGCCTCTGAAAGGCTACAAGCTCATGGGAAAGAAAGACCCCAGATAATCTGTTAC | | |
| | CCAGAAACTGGTCACTGTATTGACCCACCTTATTTTCCTCCTTCTAGAGCTTCTGTGC | | |
| | ACGCTGTTTTGGGTGAGGCAATATTCTATGGAGGTGAGCCAAAGGCTCACTCAAAGGC | | |
| | ACAGGTAGATGCCTGGCAGCAAATTCAAACTTTCTTCCATAAACATCTCAATGGTAAA | | |
| | AAATCTGTCAAGCACAGCAAAATATAACATTGTAG | | |
| | ORF Start: ATG at 9 | | ORF Stop: TAA at 1011 |
| | SEQ ID NO:90 | 334 aa | MW at 36926.0 kD |
| NOV25b, | MAATLILEPAGRCCWDEPLRIAVRGLAPEQPVTLRTSLRDEEGALFRAHARYRADASN | | |
| CG59311-02 Protein Sequence | | | |
| | PGTLGGQGRGPFPGIIDLFGSSRGLCEYRASLLAGHGFAVLALAYFRFEDLPEDLNDV | | |
| | HLEYFEEAVDFMLQHPKVKGPSIALLGFSKGGDLCLSMASFLKGITATVLINACVANT | | |
| | VAPLHYKDMIIPKLVDDLGKVKITKSGFLTFMDTWSNPLEEHNHQSLVPLEKAQVPFL | | |
| | FIVGMDDQSWKSEFYAQIASERLQAHGKERPQIICYPETGHCIDPPYFPPSRASVHAV | | |
| | LGEAIFYGGEPKAHSKAQVDAWQQIQTFFHKHLNGKKSVKHSKI | | |
| | SEQ ID NO:91 | 1021 bp | |
| NOV25c, | AGATTGGGATGGCAGCGACGCTGATCCTGGAGCCCGCGGGCCGCTGCTGCTGGGACGA | | |
| CG59311-03 DNA Sequence | | | |
| | GCCGCTGCGCATCGCAGTGCGCGGCCTGGCCCCGGAGCAGCCAGTCACGCTGCGCACG | | |
| | TCCCTGCGCGACGAAGAGGGCGCGCTCTTCCGGGCCCACGCGCGCTACCGTGCCGACG | | |
| | CCTCTAATCCCGGCACTTTGGGAGGCCAAGGCAGGGGGCCCTTTCCTGGGATCATTGA | | |
| | TCTGTTTGGGAGCAGCAGGGGCCTTTGTGAATACAGGGCCAGCCTCCTGGCCGGACAT | | |
| | GGTTTTGCTGTGCTTGCCCTGGCTTATTTCAGATTTGAAGACCTCCCCGAAGATCTGA | | |
| | ATGATGTACATCTGGAGTACTTTGAAGAAGCCGTGGACTTTATGCTGCAGCATCCAAA | | |
| | GGTGAAAGGTCCTAGTATTGCGCTTCTTGGATTTTCCAAAGGAGGTGACCTGTGTCTC | | |
| | TCAATGGCTTCTTTCTTGAAGGGCATCACAGCCACTGTACTTATCAATGCCTGTGTAG | | |
| | CCAACACAGTAGCTCCTCTACATTACAAGGATATGATTATTCCTAAACTTGTCGATGA | | |
| | TCTAGGAAAAGTAAAAATCACTAAGTCAGGATTTCTCACTTTTATGGACACTTGGAGC | | |
| | AATCCACTGGAGGAACACAATCACCAAAGTCTTGTTCCATTGOAAAAGGCGCAGGTGC | | |
| | CCTTCTTGTTTATTGTTGGCATGGATGATCAAAGCTGGAAGAGTGAATTCTATGCTCA | | |
| | GATAGCCTCTGAAAGGCTACAAGCTCATGGGAAAGAAAGACCCCAGATAATCTGTTAC | | |
| | CCAGAAACTGGTCACTGTATTGACCCACCTTATTTTCCTCCTTCTAGAGCTTCTGTGC | | |
| | ACGCTGTTTTGGGTGAGGCAATATTCTATGGAGGTGAGCCAAAGGCTCACTCAAAGGC | | |
| | ACAGGTAGATGCCTGGCAGCAAATTCAAACTTTCTTCCATAAACATCTCAATGGTAAA | | |
| | AAATCTGTCAAGCACAGCAAAATATAACATTGTAG | | |
| | ORF Start: ATG at 9 | | ORF Stop: TAA at 1011 |
| | SEQ ID NO:92 | 334 aa | MW at 36926.0 kD |

| | |
|---|---|
| NOV25c, | MAATLILEPAGRCCWDEPLRIAVRGLAPEQPVTLRTSLRDEEGALFRAHARYRADASN |
| CG59311-03 Protein Sequence | |
| | PGTLGGQGRGPFPGIIDLFGSSRGLCEYRASLLAGHGFAVLALAYFRFEDLPEDLNDV |
| | HLEYFEEAVDFMLQHPKVKGPSIALLGFSKGGDLCLSMASFLKGITATVLINACVANT |
| | VAPLHYKDMIIPKLVDDLGKVKITKSGFLTFMDTWSNPLEEHNHQSLVPLEKAQVPFL |
| | FIVGMDDQSWKSEFYAQIASERLQAHGKERPQIICYPETGHCIDPPYFPPSRASVHAV |
| | LGEAIFYGGEPKAHSKAQVDAWQQIQTFFHKHLNGKKSVKHSKI |

Sequence comparison of the above protein sequences yields the following sequence relationships shown in Table 25B.

TABLE 25B
Comparison of NOV25a against NOV25b through NOV25c.

| Protein | NOV25a Residues/ | Identities/Similarities |
|---|---|---|
| Sequence | Match Residues | for the Matched Region |
| NOV25b | 154 . . . 421 | 268/268 (100%) |
| | 67 . . . 334 | 268/268 (100%) |
| NOV25c | 154 . . . 421 | 268/268 (100%) |
| | 67 . . . 334 | 268/268 (100%) |

Further analysis of the NOV25a protein yielded the following properties shown in Table 25C.

TABLE 25C
Protein Sequence Properties NOV25a

| | |
|---|---|
| PSort | 0.4500 probability located in cytoplasm; 0.3630 probability |
| analysis: | located in microbody (peroxisome); 0.1958 probability located |
| | in lysosome (lumen); 0.1000 probability located in |
| | mitochondrial matrix space |
| SignalP | No Known Signal Sequence Predicted |
| analysis: | |

A search of the NOV25a protein against the Geneseq database, a proprietary database that contains sequences published in patents and patent publication, yielded several homologous proteins shown in Table 25D.

TABLE 25D
Geneseq Results for NOV25a

| | | NOV25a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAM41490 | Human poly- | 1 . . . 421 | 288/421 | e−175 |
| | peptide SEQ ID | | (68%) | |
| | NO 6421—Homo | 74 . . . 494 | 347/421 | |
| | sapiens, 494 aa. | | (82%) | |
| | Jul. 26, 2001] | | | |
| AAM39704 | Human poly- | 1 . . . 421 | 288/421 | e−175 |
| | peptide SEQ ID | | (68%) | |
| | NO 2849—Homo | 63 . . . 483 | 346/421 | |
| | sapiens, 483 aa. | | (81%) | |
| | [WO200153312- | | | |
| | A1, Jul. 26, 2001] | | | |
| AAY71112 | Human Hydrolase | 1 . . . 421 | 288/421 | e−175 |
| | protein-10 | | (68%) | |
| | (HYDRL-10)— | 63 . . . 483 | 346/421 | |
| | Homo sapiens, | | (81%) | |
| | 483 aa. | | | |
| | [WO200028045- | | | |
| | A2, May 18, | | | |
| | 2000] | | | |
| AAB93479 | Human protein | 1 . . . 421 | 287/421 | e−175 |
| | sequence SEQ ID | | (68%) | |
| | NO:12766— | 63 . . . 483 | 346/421 | |
| | Homo sapiens, | | (82%) | |
| | 483 aa. | | | |
| | [EP1074617-A2, | | | |
| | Feb. 7, 2001] | | | |
| AAY07932 | Human secreted | 241 . . . 421 | 181/181 | e−105 |
| | protein fragment | | (100%) | |
| | encoded from | 1 . . . 181 | 181/181 | |
| | gene 81—Homo | | (100%) | |
| | sapiens, 182 aa. | | | |
| | [WO9918208-A1, | | | |
| | Apr. 15, 1999] | | | |

In a BLAST search of public sequence databases, the NOV25a protein was found to have homology to the proteins shown in the BLASTP data in Table 25E.

TABLE 25E
Public BLASTP Results for NOV25a

| | | NOV25a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| P49753 | Peroxisomal acyl- | 1 . . . 421 | 288/421 (68%) | e−175 |
| | coenzyme A | 1 . . . 421 | 347/421 (82%) | |
| | thioester hydrolase | | | |
| | 2 (EC 3.1.2.2) | | | |
| | (Peroxisomal long- | | | |
| | chain acyl-coA | | | |
| | thioesterase 2) | | | |
| | (ZAP128)—Homo | | | |
| | sapiens (Human), | | | |
| | 421 aa. | | | |
| Q9QYR7 | Peroxisomal acyl- | 1 . . . 421 | 264/421 (62%) | e−157 |
| | coenzyme A | 12 . . . 432 | 331/421 (77%) | |
| | thioester hydrolase | | | |
| | 2 (EC 3.1.2.2) | | | |
| | (Peroxisomal long- | | | |
| | chain acyl-coA | | | |
| | thioesterase 2) | | | |
| | (PTE-Ia)—Mus | | | |
| | musculus (Mouse), | | | |
| | 432 aa. | | | |
| O88267 | Cytosolic acyl | 1 . . . 421 | 268/421 (63%) | e−153 |
| | coenzyme A | 1 . . . 419 | 318/421 (74%) | |
| | thioester hydrolase, | | | |
| | inducible | | | |
| | (EC 3.1.2.2) (Long | | | |
| | chain acyl-CoA | | | |
| | thioester hydrolase) | | | |
| | (Long chain acyl- | | | |
| | CoA hydrolase) | | | |
| | (CTE-I) (LACH2) | | | |
| | (ACH2) Rattus | | | |
| | norvegicus (Rat), | | | |
| | 419 aa. | | | |
| Q9QYR9 | Acyl coenzyme A | 3 . . . 413 | 264/411 (64%) | e−153 |
| | thioester hydrolase, | 44 . . . 452 | 321/411 (77%) | |
| | mitochondrial | | | |
| | precursor | | | |
| | (EC 3.1.2.2) (Very- | | | |
| | long-chain acyl- | | | |
| | CoA thioesterase) | | | |
| | (MTE-I)—Mus | | | |
| | musculus (Mouse), | | | |
| | 453 aa. | | | |
| O55137 | Cytosolic acyl | 1 . . . 413 | 262/413 (63%) | e−153 |
| | coenzyme A | 1 . . . 411 | 319/413 (76%) | |
| | thioester (Long | | | |
| | chain acyl-CoA | | | |
| | thioester hydrolase) | | | |
| | (Long chain acyl- | | | |
| | CoA hydrolase) | | | |
| | (CTE-I)—Mus | | | |
| | musculus (Mouse), | | | |
| | 419 aa. | | | |

PFam analysis predicts that the NOV25a protein contains the domains shown in the Table 25F.

TABLE 25F
Domain Analysis of NOV25a

| | | | |
|---|---|---|---|
| | NOV25a | Identities/Similarities | Expect |
| Pfam Domain | Match Region | for the Matched Region | Value |

No Significant Matches Found

### Example 26

The NOV26 clone was analyzed, and the nucleotide and predicted polypeptide sequences are shown in Table 26A.

TABLE 26A
NOV26 Sequence Analysis

| | SEQ ID NO:93 | 1375 bp | |
|---|---|---|---|
| NOV26a, | GGGACGCCGGACGCCGTCCGGACATTCGGCGCGCTTGCCACGATCTTGGACGGGTCTC | | |
| CG59309-01 DNA Sequence | | | |
| | GGGCCTCGACCTTTGAATTCCCCGCTCCGGCTCCAAGATGTCAGCAACGCTGATCCTG | | |
| | GAGCCCCCAGGCCGCTGCTGCTGGAACGAGCCGGTGCGCATTGCCGTGCGCGGCCTGG | | |
| | CCCCGGAGCAGCGGGTTACGCTGCGCGCGTCCCTGCGCGACGAGAAGGGCGCGCTCTT | | |
| | CCGGGCCCACGCGCGCTACTGCGCCGACGCCCGCGGCGAGCTGGACCTGGAGCGCGCA | | |
| | CCCGCGCTGGGCGGCAGCTTCGCGGGACTCGAGCCCATGGGGCTGCTCTGGGCCCTGG | | |
| | AACCCGAGAAGCCTTTTTGGCGCTTCCTGAAGCGGGACGTACAGATTCCTTTTGTCGT | | |
| | GGAGTTGGAGGTGCTGGACGGCCACGACCCCGAGCCTGGACGGCTGCTGTGCCAGGCG | | |
| | CAGCACGAGCGCCACTTCCTCCCGCCAGGGGTGCGGCGCCAGTCGGTGCGAGCGGGCC | | |
| | GGGTGCGCGCCACGCTCTTCCTGCCGCCAGGTGAGCCTGGACCCTTCCCAGGGATCAT | | |
| | TGACATCTTTGGTATTGGAGGGGGCCTCTTGGAATATCGAGCCAGCCTCCTTGCTGGC | | |
| | CATGGCTTTGCCACGTTGGCTCTAGCTTATTATAACTTTGAAGATCTCCCCAATAACA | | |
| | TGGACAACATATCCCTGGAGTACTTCGAAGAAGCCGTATGCTACATGCTTCAACATCC | | |
| | CCAGGTTAAAGGCCCAGGCATTGGGCTTTTGGGCATTTCTCTAGGAGCTGATATTTGT | | |
| | CTCTCAATGGCCTCATTCTTGAAGAATGTCTCAGCCACAGTTTCCATCAATGGATCTG | | |
| | GGATCAGTGGGAACACAGCCATCAACTATAAGCACAGTAGCATTCCACCATTGGGCTA | | |
| | TGACCTGAGGAGAATCAAGGTAGCTTTCTCAGGCCTCGTGGACATTGTGGATATAAGG | | |
| | AATGCTCTCGTAGGAGGGTACAAGAACCCCAGCATGATTCCAATAGAGAAGGCCCAGG | | |
| | GGCCCATCCTGCTCATTGTTGGTCAGGATGACCATAACTGGAGAAGTGAGTTGTATGC | | |
| | CCAAACAGTCTCTGAACGGTTACAGGCCCATGGAAAGGAAAAACCCCAGATCATCTGT | | |
| | TACCCTGGGACTGGGCATTACATCGAGCCTCCTTACTTCCCCCTGTGCCCAGCTTCCC | | |
| | TTCACAGATTACTGAACAAACATGTTATATGGGGTGGGGAGCCCAGGGCTCATTCTAA | | |
| | GGCCCAGGAAGATGCCTGGAAGCAAATTCTAGCCTTCTTCTGCAAACACCTGGGAGGT | | |
| | ACCCAGAAAACAGCTGTCCCTAAATTGTAATGCATTTGTCT | | |
| | ORF Start: ATG at 96 | | ORF Stop: TAA at 1362 |
| | SEQ ID NO:94 | 422 aa | MW at 46455.1 kD |

| | |
|---|---|
| NOV26a, | MSATLILEPPGRCCWNEPVRIAVRGLAPEQRVTLRASLRDEKGALFRAHARYCADARG |
| CG59309-01 Protein Sequence | |
| | ELDLERAPALGGSFAGLEPMGLLWALEPEKPFWRFLKRDVQIPFVVELEVLDGHDPEP |
| | GRLLCQAQHERHFLPPGVRRQSVRAGRVRATLFLPPGEPGPFPGIIDIFGIGGGLLEY |
| | RASLLAGHGFATLALAYYNFEDLPNNMDNISLEYFEEAVCYMLQHPQVKGPGIGLLGI |
| | SLGADICLSMASFLKNVSATVSINGSGISGNTAINYKHSSIPPLGYDLRRIKVAFSGL |
| | VDIVDIRNALVGGYKNPSMIPIEKAQGPILLIVGQDDHNWRSELYAQTVSERLQAHGK |
| | EKPQIICYPGTGHYIEPPYFPLCPASLHRLLNKHVIWGGEPRAHSKAQEDAWKQILAF |
| | FCKHLGGTQKTAVPKL |

Further analysis of the NOV26a protein yielded the following properties shown in Table 26B.

TABLE 26B
Protein Sequence Properties NOV26a

| | |
|---|---|
| PSort | 0.4500 probability located in cytoplasm; 0.2585 probability |
| analysis: | located in lysosome (lumen); 0.1940 probability located in |
| | microbody (peroxisome); 0.1000 probability located in |
| | mitochondrial matrix space |
| SignalP | No Known Signal Sequence Predicted |
| analysis: | |

A search of the NOV26a protein against the Geneseq database, a proprietary database that contains sequences published in patents and patent publication, yielded several homologous proteins shown in Table 26C.

TABLE 26C
Geneseq Results for NOV26a

| | | NOV26a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAM41490 | Human poly- | 1 . . . 422 | 296/422 (70%) | e−179 |
| | peptide SEQ ID | 74 . . . 494 | 341/422 (80%) | |
| | NO 6421—Homo | | | |
| | sapiens, 494 aa. | | | |
| | [WO200153312- | | | |
| | A1, Jul. 26, 2001] | | | |
| AAM39704 | Human poly- | 1 . . . 422 | 296/422 (70%) | e−179 |
| | peptide SEQ ID | 63 . . . 483 | 341/422 (80%) | |
| | NO 2849—Homo | | | |
| | sapiens, 483 aa. | | | |
| | [WO200153312- | | | |
| | A1, Jul. 26, 2001] | | | |
| AAY71112 | Human Hydrolase | 1 . . . 422 | 296/422 (70%) | e−179 |
| | protein-10 | 63 . . . 483 | 341/422 (80%) | |
| | (HYDRL-10)— | | | |
| | Homo sapiens, | | | |
| | 483 aa. | | | |
| | [WO200028045- | | | |
| | A2, May 18, | | | |
| | 2000] | | | |
| AAB93479 | Human protein | 1 . . . 422 | 295/422 (69%) | e−178 |
| | sequence SEQ ID | 63 . . . 483 | 340/422 (79%) | |
| | NO:12766— | | | |
| | Homo sapiens, | | | |
| | 483 aa. | | | |
| | [EP1074617-A2, | | | |
| | Feb. 7, 2001] | | | |
| AAY07932 | Human secreted | 242 . . . 422 | 93/181 (51%) | 2e−48 |
| | protein fragment | 1 . . . 181 | 123/181 (67%) | |
| | encoded from | | | |
| | gene 81—Homo | | | |
| | sapiens, 182 aa. | | | |
| | [WO9918208-A1, | | | |
| | Apr. 15, 1999] | | | |

In a BLAST search of public sequence databases, the NOV26a protein was found to have homology to the proteins shown in the BLASTP data in Table 26D.

TABLE 26D
Public BLASTP Results for NOV26a

| | | NOV26a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9QYR8 | PEROXISOMAL | 1 . . . 422 | 312/422 (73%) | 0.0 |
| | LONG CHAIN | 1 . . . 421 | 362/422 (84%) | |
| | ACYL-COA THIO- | | | |
| | ESTERASE IB— | | | |
| | Mus musculus | | | |
| | (Mouse), 421 aa. | | | |
| P49753 | Peroxisomal acyl- | 1 . . . 422 | 296/422 (70%) | e−178 |
| | coenzyme A | 1 . . . 421 | 341/422 (80%) | |
| | thioester hydrolase | | | |
| | 2 (EC 3.1.2.2) | | | |
| | (Peroxisomal long- | | | |
| | chain acyl-coA | | | |
| | thioesterase 2) | | | |
| | (ZAP128)—Homo | | | |
| | sapiens (Human), | | | |
| | 421 aa. | | | |
| Q9QYR7 | Peroxisomal acyl- | 1 . . . 422 | 281/424 (66%) | e−163 |
| | coenzyme A | 12 . . . 432 | 333/424 (78%) | |
| | thioester hydrolase | | | |
| | 2 (EC 3.1.2.2) | | | |
| | (Peroxisomal long- | | | |
| | chain acyl-coA | | | |
| | thioesterase 2) | | | |
| | (PTE-Ia)—Mus | | | |
| | musculus (Mouse), | | | |
| | 432 aa. | | | |
| O55137 | Cytosolic acyl | 1 . . . 422 | 275/423 (65%) | e−162 |
| | coenzyme A | 1 . . . 419 | 330/423 (78%) | |
| | thioester hydrolase, | | | |
| | inducible | | | |
| | (EC 3.1.2.2) (Long | | | |
| | chain acyl-CoA | | | |
| | thioester hydrolase) | | | |
| | (Long chain acyl- | | | |
| | CoA hydrolase) | | | |
| | (CTE-I)—Mus | | | |
| | musculus (Mouse), | | | |
| | 419 aa. | | | |
| O88267 | Cytosolic acyl | 1 . . . 422 | 276/423 (65%) | e−162 |
| | coenzyme A | 1 . . . 419 | 329/423 (77%) | |
| | thioester hydrolase, | | | |
| | inducible | | | |
| | (EC 3.1.2.2) (Long | | | |
| | chain acyl-CoA | | | |
| | thioester hydrolase) | | | |
| | (Long chain acyl- | | | |
| | CoA hydrolase) | | | |
| | (CTE-I) (LACH2) | | | |
| | (ACH2) Rattus | | | |
| | norvegicus (Rat), | | | |
| | 419 aa. | | | |

PFam analysis predicts that the NOV26a protein contains the domains shown in the Table 26E.

TABLE 26E
Domain Analysis of NOV26a

| | | Identities/ | |
|---|---|---|---|
| | NOV26a | Similarities | |
| | Match | for the | |
| Pfam Domain | Region | Matched Region | Expect Value |
| DLH: domain 1 of 2 | 144 . . . 188 | 17/52 (33%) | 63 |
| | | 32/52 (62%) | |
| DLH: domain 2 of 2 | 394 . . . 411 | 9/18 (50%) | 2.6 |
| | | 13/18 (72%) | |

### Example 27

The NOV27 clone was analyzed, and the nucleotide and predicted polypeptide sequences are shown in Table 27A.

TABLE 27A
NOV27 Sequence Analysis

| | SEQ ID NO:95 | 1333 bp | |
|---|---|---|---|
| NOV27a, | CCTGGCCCCCAAGCTCCCCACTCTGGTGCCCCGAGCAGCCCTGTGGGCAAGCAGCCGC | | |
| CG57364-01 DNA Sequence | | | |
| | CGCCATGGCCGAGCACCTGGAGCTGCTGGCAGAGATGCCCATGGTGGGCAGGATGAGC | | |
| | ACACAGGAGCGGCTGAAGCATGCCCAGAAGCGGCGCGCCCAGCAGGTGAAGATGTGGG | | |
| | CCCAGGCTGAGAAGGAGGCCCAGGGCAAGAAGGGTCCTGGGGAGCGTCCCCGGAAGGA | | |
| | GGCAGCCAGCCAAGGGCTCCTGAAGCAGGTCCTCTTCCCTCCCAGTGTTGTCCTTCTG | | |
| | GAGGCCGCTGCCCGAAATGACCTGGAAGAAGTCCGCCAGTTCCTTGGGAGTGGGGTCA | | |
| | GCCCTGACTTGGCCAACGAGGACGGCCTGACGGCCCTGCACCAGTGCTGCATTGATGA | | |
| | TTTCCGAGAGATGGTGCAGCAGCTCCTGGAGGCTGGGGCCAACATCAATGCCTGTGAC | | |
| | AGTGAGTGCTGGACGCCTCTGCATGCTGCGGCCACCTGCGGCCACCTGCACCTGGTGG | | |
| | AGCTGCTCATCGCCAGTGGCGCCAATCTCCTGGCGGTCAACACCGACGGGAACATGCC | | |
| | CTATGACCTGTGTGATGATGAGCAGACGCTGGACTGCCTGGAGACTGCCATGGCCGAC | | |
| | CGTGGCATCACCCAGGACAGCATCGAGGCCGCCCGGGCCGTGCCAGAACTGCGCATGC | | |
| | TGGACGACATCCGGAGCCGGCTGCAGGCCGGGGCAGACCTCCATGCCCCCCTGGACCA | | |
| | CGGGGCCACGCTGCTGCACGTCGCAGCCGCCAACGGGTTCAGCGAGGCGGCTGCCCTG | | |
| | CTGCTGGAACACCGAGCCAGCCTGAGCGCTAAGGACCAAGACGGCTGGGAGCCGCTGC | | |
| | ACGCCGCGGCCTACTGGGGCCAGGTGCCCCTGGTGGAGCTGCTCGTGGCGCACGGGGC | | |
| | CGACCTGAACGCAAAGTCCCTGATGGACGAGACGCCCCTTGATGTGTGCGGGGACGAG | | |
| | GAGGTGCGGGCCAAGCTGCTGGAGCTGAAGCACAAGCACGACGCCCTCCTGCGCGCCC | | |
| | AGAGCCGCCAGCGCTCCTTGCTGCGCCGCCGCACCTCCAGCGCCGGCAGCCGCGGGAA | | |
| | GGTGGTGAGGCGGGATGAGCCTAACCCAGCGCAGCGGCTGACGCATGTCCCAGAAGCG | | |
| | GCGCGCCCAGCAGGTGAAGATGTGGGCCCAGGCTGAGAAGGAGGCCCAGGGCAAGAAG | | |
| | GGTCCTGGGGAGCGTCCCCGGAAGGAGGCAGCCAGCCAAGGGCTCCTGAAGCAGGTCC | | |
| | TCTTCCCTCCCAGTGTTGTCCTTCTGGAGGCCGCTGCCCGAAATGACCTGGAAGAAG | | |
| | ORF Start: ATG at 63 | | ORF Stop: TGA at 1194 |
| | SEQ ID NO:96 | 377 aa | MW at 41019.9 kD |

| | |
|---|---|
| NOV27a, | MAEHLELLAEMPMVGRMSTQERLKHAQKRRAQQVKMWAQAEKEAQGKKGPGERPRKEA |
| CG57364-01 Protein Sequence | |
| | ASQGLLKQVLFPPSVVLLEAAARNDLEEVRQFLGSGVSPDLANEDGLTALHQCCIDDF |
| | REMVQQLLEAGANINACDSECWTPLHAAATCGHLHLVELLIASGANLLAVNTDGNMPY |
| | DLCDDEQTLDCLETAMADRGITQDSIEAARAVPELRMLDDIRSRLQAGADLHAPLDHG |
| | ATLLHVAAANGFSEAAALLLEHRASLSAKDQDGWEPLHAAAYWGQVPLVELLVAHGAD |
| | LNAKSLMDETPLDVCGDEEVRAKLLELKHKHDALLRAQSRQRSLLRRRTSSAGSRGKV |
| | VRRDEPNPAQRLTHVPEAARPAGEDVGPG |

Further analysis of the NOV27a protein yielded the following properties shown in Table 27B.

TABLE 27B
Protein Sequence Properties NOV27a

| | |
|---|---|
| PSort | 0.3000 probability located in microbody (peroxisome); 0.3000 |
| analysis: | probability located in nucleus; 0.1547 probability located in |
| | lysosome (lumen); 0.1000 probability located in mitochondrial |
| | matrix space |
| SignalP | No Known Signal Sequence Predicted |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | | Identities/ | |
|---|---|---|---|---|
| | | NOV27a | Similarities | |
| | Protein/ | Residues/ | for the | |
| Geneseq | Organism/Length | Match | Matched | Expect |
| Identifier | [Patent #, Date] | Residues | Region | Value |
| AAM40636 | Human polypeptide | 89 . . . 351 | 262/263 | e−151 |
| | SEQ ID NO 5567— | | (99%) | |
| | Homo sapiens, | 1 . . . 263 | 263/263 | |
| | 440 aa. | | (99%) | |
| | [WO200153312-A1, | | | |
| | Jul. 26, 2001] | | | |
| AAM38850 | Human polypeptide | 119 . . . 351 | 233/233 | e−132 |
| | SEQ ID NO 1995— | | (100%) | |
| | Homo sapiens, | 1 . . . 233 | 233/233 | |
| | 410 aa. | | (100%) | |
| | [WO200153312-A1, | | | |
| | Jul. 26, 2001] | | | |
| AAM78864 | Human protein SEQ | 1 . . . 351 | 209/351 | e−118 |
| | ID NO 1526— | | (59%) | |
| | Homo sapiens, | 1 . . . 348 | 265/351 | |
| | 567 aa. | | (74%) | |
| | [WO200157190-A2, | | | |
| | Aug. 9, 2001] | | | |
| ABB11817 | Human KIAA0823 | 45 . . . 354 | 173/316 | 3e−94 |
| | protein homologue, | | (54%) | |
| | SEQ ID NO: | 3 . . . 318 | 226/316 | |
| | 2187—Homo | | (70%) | |
| | sapiens, 536 aa. | | | |
| | [WO200157188-A2, | | | |
| | Aug. 9, 2001] | | | |
| AAM79848 | Human protein | 45 . . . 354 | 173/316 | 3e−94 |
| | SEQ ID NO 3494— | | (54%) | |
| | Homo sapiens, | 3 . . . 318 | 226/316 | |
| | 536 aa. | | (70%) | |
| | [WO200157190-A2, | | | |
| | Aug. 9, 2001] | | | |

In a BLAST search of public sequence databases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96134 | UNKNOWN | 1 . . . 351 | 351/351 (100%) | 0.0 |
| | (PROTEIN FOR | 1 . . . 351 | 351/351 (100%) | |
| | MGC:14333)— | | | |
| | Homo sapiens | | | |
| | (Human), 528 aa. | | | |
| Q923M0 | MYOSIN | 1 . . . 351 | 301/351 (85%) | e−171 |
| | PHOSPHATASE | 1 . . . 351 | 320/351 (90%) | |
| | TARGETING | | | |
| | SUBUNIT 3 | | | |
| | MYPT3—Mus | | | |
| | musculus | | | |
| | (Mouse), 524 aa | | | |
| | (fragment). | | | |
| AAL62093 | PROTEIN PHOS- | 1 . . . 351 | 210/351 (59%) | e−118 |
| | PHATASE 1 | 1 . . . 348 | 266/351 (74%) | |
| | REGULATORY | | | |
| | SUBUNIT 16B— | | | |
| | Mus musculus | | | |
| | (Mouse), 568 aa. | | | |
| Q95N27 | CAAX BOX | 1 . . . 351 | 210/351 (59%) | e−118 |
| | PROTEIN | 1 . . . 348 | 266/351 (74%) | |
| | TIMAP—Bos | | | |
| | taurus (Bovine), | | | |
| | 568 aa. | | | |
| Q96T49 | CAAX BOX | 1 . . . 351 | 209/351 (59%) | e−117 |
| | PROTEIN | 1 . . . 348 | 265/351 (74%) | |
| | TIMAP—Homo | | | |
| | sapiens (Human), | | | |
| | 567 aa. | | | |

PFam analysis predicts that the NOV27a protein contains the domains shown in the Table 27E.

TABLE 27E
Domain Analysis of NOV27a

| | NOV27a | Identities/Similarities | Expect |
|---|---|---|---|
| Pfam Domain | Match Region | for the Matched Region | Value |
| ank: domain 1 of 5 | 70 . . . 102 | 8/33 (24%) | 99 |
| | | 20/33 (61%) | |
| ank: domain 2 of 5 | 103 . . . 135 | 16/33 (48%) | 7.1e−08 |
| | | 26/33 (79%) | |
| ank: domain 3 of 5 | 136 . . . 168 | 15/33 (45%) | 2.9e−07 |
| | | 26/33 (79%) | |
| ank: domain 4 of 5 | 231 . . . 263 | 16/33 (48%) | 2e−06 |
| | | 24/33 (73%) | |
| ank: domain 5 of 5 | 264 . . . 296 | 16/33 (48%) | 2.7e−08 |
| | | 27/33 (82%) | |

### Example 28

The NOV28 clone was analyzed, and the nucleotide and predicted polypeptide sequences are shown in Table 28A.

TABLE 28A
NOV28 Sequence Analysis

| | SEQ ID NO:97 | 1719 bp | |
|---|---|---|---|
| NOV28a, | CGGGCACAGGCTCACCCTCGAGTGGCACAGGAATCCCAGGTAGATGACGGCGGCCGCG | | |
| CG59348-01 DNA Sequence | | | |
| | GCTGGTGCTGCAGGGTCGCCAGCTCCCGCGGCAGCGGCCGGCGCCCCGGGATCTGGGG | | |
| | GCGCACCCTCAGGGTCGCAGGGGGTGCTGATCGGGGACAGGCTGTACTCCGGGGTGCT | | |
| | CATCACCTTGGAGAACTGCCTCCTGCCTGACGACAAGCTCCGTTTCACGCCGTCCATG | | |
| | TCGAGCGGCCTCGACACCGACACAGAGACCGACCTCCGCGTGGTGGGCTGCGAGCTCA | | |
| | TCCAGGCGGCCGGTATCCTGCTCCGCCTGCCGCAGGTGGCCATGGCTACCGGGCAGGT | | |
| | GTTGTTCCAGCGGTTCTTTTATACCAAGTCCTTCGTGAAGCACTCCATGGAGCATGTG | | |
| | TCAATGGCCTGTGTCCACCTGGCTTCCAAGATAGAAGAGGCCCCAAGACGCATACGGG | | |
| | ACGTCATCAATGTGTTTCACCGCCTTCGACAGCTGAGAGACAAAAAGAAGCCCGTGCC | | |
| | TCTACTACTGGATCAAGATTATGTTAATTTAAAGAACCAAATTATAAAGGCGGAAAGA | | |
| | CGAGTTCTCAAAGAGTTGGGTTTCTGCGTCCATGTGAAGCATCCTCATAAGATAATCG | | |
| | TTATGTACCTTCAGGTGTTAGAGTGTGAGCGTAACCAACACCTGGTCCAGACCTCATG | | |
| | GAATTACATGAACGACAGCCTTCGCACCGACGTCTTCGTGCGGTTCCAGCCAGAGAGC | | |
| | ATCGCCTGTGCCTGCATTTATCTTGCTGCCCGGACGCTGGAGATCCCTTTGCCCAATC | | |
| | GTCCCCATTGGTTTCTTTTGTTTGGAGCAACTGAAGAAGAAATTCAGGAAATCTGCTT | | |
| | AAAGATCTTGCAGCTTTATGCTCGGAAAAAGGTTGATCTCACACACCTGGAGGGTGAA | | |
| | GTGGAAAAAAGAAAGCACGCTATCGAAGAGGCAAAGGCCCAAGCCCGGGGCCTGTTGC | | |
| | CTGGGGGCACACAGGTGCTGGATGGTACCTCGGGGTTCTCTCCTGCCCCCAAGCTGGT | | |
| | GGAATCCCCCAAAGAAGGTAAAGGGAGCAAGCCTTCCCCACTGTCTGTGAAGAACACC | | |
| | AAGAGGAGGCTGGAGGGCGCCAAGAAAGCCAAGGCGGACAGCCCCGTGAACGGCTTGC | | |
| | CAAAGGGGCGAGAGAGTCGGAGTCGGAGCCGGAGCCGTGAGCAGAGCTACTCGAGGTC | | |
| | CCCATCCCGATCAGCGTCTCCTAAGAGGAGGAAAAGTGACAGCGGCTCCACATCTGGT | | |
| | GGGTCCAAGTCGCAGAGCCGCTCCCGGAGCAGGAGTGACTCCCCACCGAGACAGGCCC | | |
| | CCCGCAGCGCTCCCTACAAAGGCTCTGAGATTCGGGGCTCCCGGAAGTCCAAGGACTG | | |
| | CAAGTACCCCCAGAAGCCACACAAGTCTCGGAGCCGGAGTTCTTCCCGTTCTCGAAGC | | |
| | AGGTCACGGGAGCGGGCGGATAATCCGGGAAAATACAAGAAGAAAAGTCATTACTACA | | |
| | GAGATCAGCGACGAGAGCGCTCGAGGTCGTATGAACGCACAGGCCGTCGCTATGAGCG | | |
| | GGACCACCCTGGGCACAGCAGGCATCGGAGGTGACACGTGCTTCAGACCGGTCTGGGG | | |
| | TGCGGCGCACACCTGGGCCCGTGCAGGGCTCAGCTCGGCAGCAGCTCTGAGGGCAGCT | | |
| | CAATGAAAAAGTGAATGCACACGCCCTTGTTGGCGTG | | |
| | ORF Start: ATG at 44 | | ORF Stop: TGA at 1598 |
| | SEQ ID NO:98 | 518 aa | MW at 58034.5 kD |

| | |
|---|---|
| NOV28a, | MTAAAAGAAGSAAPAAAAGAPGSGGAPSGSQGVLIGDRLYSGVLITLENCLLPDDKLR |
| CG59348-01 Protein Sequence | |
| | FTPSMSSGLDTDTETDLRVVGCELIQAAGILLRLPQVAMATGQVLFQRFFYTKSFVKH |
| | SMEHVSMACVHLASKIEEAPRRIRDVINVFHRLRQLRDKKKPVPLLLDQDYVNLKNQI |
| | IKAERRVLKELGFCVHVKHPHKIIVMYLQVLECERNQHLVQTSWNYMNDSLRTDVFVR |
| | FQPESIACACIYLAARTLEIPLPNRPHWFLLFGATEEEIQEICLKILQLYARKKVDLT |
| | HLEGEVEKRKHAIEEAKAQARGLLPGGTQVLDGTSGFSPAPKLVESPKEGKGSKPSPL |
| | SVKNTKRRLEGAKKAKADSPVNGLPKGRESRSRSRSREQSYSRSPSRSASPKRRKSDS |
| | GSTSGGSKSQSRSRSRSDSPPRQAPRSAPYKGSEIRGSRKSKDCKYPQKPHKSRSRSS |
| | SRSRSRSRERADNPGKYKKKSHYYRDQRRERSRSYERTGRRYERDHPGHSRHRR |

Further analysis of the NOV28a protein yielded the following properties shown in Table 28B.

TABLE 28B
Protein Sequence Properties NOV28a

| | |
|---|---|
| Psort | 0.5500 probability located in endoplasmic reticulum |
| analysis: | (membrane); 0.2400 probability located in nucleus; 0.1900 |
| | probability located in lysosome (lumen); 0.1000 probability |
| | located in endoplasmic reticulum (lumen) |
| SignalP | No Known Signal Sequence Predicted |
| analysis: | |

A search of the NOV28a protein against the Geneseq database, a proprietary database that contains sequences published in patents and patent publication, yielded several homologous proteins shown in Table 28C.

TABLE 28C
Geneseq Results for NOV28a

| | | NOV28a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAM94028 | Human stomach | 221 . . . 518 | 298/298 | e−172 |
| | cancer expressed | | (100%) | |
| | polypeptide SEQ | 1 . . . 298 | 298/298 | |
| | ID NO 126— | | (100%) | |
| | Homo sapiens, | | | |
| | 298 aa. | | | |
| | [WO200109317- | | | |
| | A1, Feb. 8, 2001] | | | |
| AAG64403 | Human paneth | 221 . . . 518 | 298/298 | e−172 |
| | cell enhanced | | (100%) | |
| | expression-like | 1 . . . 298 | 298/298 | |
| | protein—Homo | | (100%) | |
| | sapiens, 298 aa. | | | |
| | [WO200138372- | | | |
| | A1, May 31, | | | |
| | 2001] | | | |
| AAB94641 | Human protein | 221 . . . 518 | 298/298 | e−172 |
| | sequence SEQ ID | | (100%) | |
| | NO:15526— | 1 . . . 298 | 298/298 | |
| | Homo sapiens, | | (100%) | |
| | 298 aa. | | | |
| | [EP1074617-A2, | | | |
| | Feb. 7, 2001] | | | |
| AAM78533 | Human protein | 2 . . . 518 | 316/526 | e−168 |
| | SEQ ID NO | | (60%) | |
| | 1195—Homo | 8 . . . 526 | 390/526 | |
| | sapiens, 526 aa. | | (74%) | |
| | [WO200157190- | | | |
| | A2, Aug. 9, | | | |
| | 2001] | | | |
| AAB94371 | Human protein | 2 . . . 518 | 316/526 | e−168 |
| | sequence SEQ ID | | (60%) | |
| | NO:14909— | 8 . . . 526 | 390/526 | |
| | Homo sapiens, | | (74%) | |
| | 526 aa. | | | |
| | [EP1074617-A2, | | | |
| | Feb. 7, 2001] | | | |

In a BLAST search of public sequence databases, the NOV28a protein was found to have homology to the proteins shown in the BLASTP data in Table 28D.

TABLE 28D
Public BLASTP Results for NOV28a

| | | NOV28a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96S94 | HYPO- | 3 . . . 518 | 516/516 (100%) | 0.0 |
| | THETICAL | 5 . . . 520 | 516/516 (100%) | |
| | 58.1 KDA | | | |
| | PROTEIN— | | | |
| | Homo sapiens | | | |
| | (Human), 520 aa. | | | |
| Q9JJA7 | BRAIN CDNA, | 1 . . . 518 | 466/519 (89%) | 0.0 |
| | CLONE MNCB- | 1 . . . 518 | 482/519 (92%) | |
| | 5160, SIMILAR | | | |
| | TO MUS | | | |
| | MUSCULUS | | | |
| | PANETH CELL | | | |
| | ENHANCED | | | |
| | EXPRESSION | | | |
| | PCEE MRNA— | | | |
| | Mus musculus | | | |
| | (Mouse), 518 aa. | | | |
| Q9UK58 | CYCLIN L | 2 . . . 518 | 316/526 (60%) | e−167 |
| | ANIA-6A— | 8 . . . 526 | 390/526 (74%) | |
| | Homo sapiens | | | |
| | (Human), 526 aa. | | | |
| Q9R1Q2 | CYCLIN ANIA- | 2 . . . 518 | 312/526 (59%) | e−165 |
| | 6A—Rattus | 9 . . . 527 | 391/526 (74%) | |
| | norvegicus | | | |
| | (Rat), 527 aa. | | | |
| Q9WV44 | CYCLIN ANIA- | 3 . . . 518 | 314/526 (59%) | e−162 |
| | 6A—Mus | 15 . . . 531 | 385/526 (72%) | |
| | musculus | | | |
| | (Mouse), 531 aa. | | | |

PFam analysis predicts that the NOV28a protein contains the domains shown in the Table 28E.

TABLE 28E
Domain Analysis of NOV28a

| | | Identities/ | | |
|---|---|---|---|---|
| | NOV28a | Similarities for | | |
| | Match | the Matched | | Expect |
| Pfam Domain | Region | Region | | Value |
| cyclin: domain 1 of 1 | 46 . . . 190 | 28/163 | (17%) | 0.0022 |
| | | 86/163 | (53%) | |
| Srg: domain 1 of 1 | 221 . . . 230 | 4/10 | (40%) | 6.7 |
| | | 10/10 | (100%) | |
| transcript_fac2: domain 1 | 235 . . . 253 | 12/19 | (63%) | 0.86 |
| of 1 | | 15/19 | (79%) | |
| cyclin_C: domain 1 of 1 | 196 . . . 311 | 22/139 | (16%) | 2.6 |
| | | 65/139 | (47%) | |

### Example 29

The NOV29 clone was analyzed, and the nucleotide and predicted polypeptide sequences are shown in Table 29A.

TABLE 29A
NOV29 Sequence Analysis

| | SEQ ID NO:99 | 1069 bp | |
|---|---|---|---|
| NOV29a, | CGGGGCCTGGTCGGCAGCTGGGCCGCCATGGAGTCCACGCTGGGCGCGGGCATCGTGA | | |
| CG59245-01 DNA Sequence | | | |
| | TAGCCGAGGCGCTACAGAACCAGCTAGCCTGGCTGGAGAACGTGTGGCTCTGGATCAC | | |
| | CTTTCTGGGCGATCCCAAGATCCTCTTTCTGTTCTACTTCCCCGCGGCCTACTACGCC | | |
| | TCCCGCCGTGTGGGCATCGCGGTGCTCTGGATCAGCCTCATCACCGAGTGGCTCAACC | | |
| | TCATCTTCAAGTGGTTTCTTTTTGGAGACAGGCCCTTTTGGTGGGTCCATGAGTCTGG | | |
| | TTACTACAGCCAGGCTCCAGCCCAGGTTCACCAGTTCCCCTCTTCTTGTGAGACTGGT | | |
| | CCAGGTGGCAGCCCTTCTGGACACTGCATGATCACAGGAGCAGCCCTCTGGCCCATAA | | |
| | TGACGGCCCTGTCTTCGCAGGTGCGCTGGGTAAGGGTGATGCCTAGCCTGGCTTATTG | | |
| | CACCTTCCTTTTGGCGGTTGGCTTGTCGCGAATCTTCATCTTAGCACATTTCCCTCAC | | |
| | CAGGTGCTGGCTGGCCTAATAACTGGTTGGCTGATGACTCCCCGAGTGCCTATGGAGC | | |
| | GGGAGCTAAGCTTCTATGGGTTGACTGCACTGGCCCTCATGCTAGGCACCAGCCTCAT | | |
| | CTATTGGACCCTCTTTACACTGGGCCTGGATCTTTCTTGGTCCATCAGCCTAGCCTTC | | |
| | AAGTGGTGTGAGCGGCCTGAGTGGATACACGTGGATAGCCGGCCCTTTGCCTCCCTGA | | |
| | GCCGTGACTCAGGGGCTGCCCTGGGCCTGGGCATTGCCTTGCACTCTCCCTGCTATGC | | |
| | CCAGGTGCGTCGGGCACAGCTGGGAAATGGCCAGAAGATAGCCTGCCTTGTGCTGGCC | | |
| | ATGGGGCTGCTGGGCCCCCTGGACTGGCTGGGCCACCCCCCTCAGATCAGCCTCTTCT | | |
| | ACATTTTCAATTTCCTCAAGTACACCCTCTGGCCATGCCTAGTCCTGGCCCTCGTGCC | | |
| | CTGGGCAGTGCACATGTTCAGTGCCCAGGAAGCACCGCCCATCCACTCTTCCTGACTT | | |
| | CTTGTGTGCCTCCCTTTCCTTTCCC | | |
| | ORF Start: ATG at 28 | | ORF Stop: TGA at 1039 |
| | SEQ ID NO:100 | 337 aa | MW at 37808.0 kD |
| NOV29a, | MESTLGAGIVIAEALQNQLAWLENVWLWITFLGDPKILFLFYFPAAYYASRRVGIAVL | | |
| CG59245-01 Protein Sequence | | | |
| | WISLITEWLNLIFKWFLFGDRPFWWVHESGYYSQAPAQVHQFPSSCETGPGGSPSGHC | | |
| | MITGAALWPIMTALSSQVRWVRVMPSLAYCTFLLAVGLSRIFILAHFPHQVLAGLITG | | |
| | WLMTPRVPMERELSFYGLTALALMLGTSLIYWTLFTLGLDLSWSISLAFKWCERPEWI | | |
| | HVDSRPFASLSRDSGAALGLGIALHSPCYAQVRRAQLGNGQKIACLVLAMGLLGPLDW | | |
| | LGHPPQISLFYIFNFLKYTLWPCLVLALVPWAVEMFSAQEAPPIHSS | | |
| | SEQ ID NO:101 | 1386 bp | |
| NOV29b, | TGAGTCTGTACTTTCCGCCCTGGAGCAAGCCGGGGCCTGGTCGGCAGCTGGGCCGCCA | | |
| CG59245-01 DNA Sequence | | | |
| | TGGAGTCCACGCTGGGCGCGGGCATCGTGATAGCCGAGGCGCTACAGAACCAGCTAGC | | |
| | CTGGCTGGAGAACGTGTGGCTCTGGATCACCTTTCTGGGCGATCCCAAGATCCTCTTT | | |
| | CTGTTCTACTTCCCCGCGGCCTACTACGCCTCCCGCCGTGTGGGCATCGCGGTGCTCT | | |
| | GGATCAGCCTCATCACCGAGTGGCTCAACCTCATCTTCAAGTGGTTTCTTTTTGGAGA | | |
| | CAGGCCCTTTTGGTGGGTCCATGAGTCTGGTTACTACAGCCAGGCTCCAGCCCAGGTT | | |
| | CACCAGTTCCCCTCTTCTTGTGAGACTGGTCCAGGCAGCCCTTCTGGACACTGCATGA | | |
| | TCACAGGAGCAGCCCTCTGGCCCATAATGACGGCCCTGTCTTCGCAGGTGGCCACTCG | | |
| | GGCCCGCAGCCGCTGGGTAAGGGTGATGCCTAGCCTGGCTTATTGCACCTTCCTTTTG | | |
| | GCGGTTGGCTTGTCGCGAATCTTCATCTTAGCACATTTCCCTCACCAGGTGCTGGCTG | | |
| | GCCTAATAACTGGCGCTGTCCTGGGCTGGCTGATGACTCCCCGAGTGCCTATGGAGCG | | |
| | GGAGCTAAGCTTCTATGGGTTGACTGCACTGGCCCTCATGCTAGGCACCAGCCTCATC | | |
| | TATTGGACCCTCTTTACACTGGGCCTGGATCTTTCTTGGTCCATCAGCCTAGCCTTCA | | |
| | AGTGGTGTGAGCGGCCTGAGTGGATACACGTGGATAGCCGGCCCTTTGCCTCCCTGAG | | |
| | CCGTGACTCAGGGGCTGCCCTGGGCCTGGGCATTGCCTTGCACTCTCCCTGCTATGCC | | |
| | CAGGTGCGTCGGGCACAGCTGGGAAATGGCCAGAAGATAGCCTGCCTTGTGCTGGCCA | | |
| | TGGGGCTGCTGGGCCCCCTGGACTGGCTGGGCCACCCCCCTCAGATCAGCCTCTTCTA | | |
| | CATTTTCAATTTCCTCAAGTACACCCTCTGGCCATGCCCAGTCCTGGCCCTCGTGCCC | | |
| | TGGGCAGTGCACATGTTCAGTGCCCAGGAAGCACCGCCCATCCACTCTTCCTGACTTC | | |
| | TTGTGTGCCTCCCTTTCCTTTCCCTCCCACAAAGCCAACACTCTGTGACCACCACACT | | |
| | CCAGGAGGCAGCCCCATCCCCTTCCAGCCCCTAAGTAGGCCCTCCCCTCCCTAAATCT | | |
| | GCTTCCGCACCACCTGGTCTTAGCCCCAAAGATGGGCCTTCTCTCTCCCAGATAAGTT | | |
| | GGTCCTCCCTCTGCCTTTCCTCTCAAGCCCCCAAAGAGCAAAGGCAACAGCAAGACCA | | |
| | GCGGGTTCTTGCAACACTGTGAGGGGCAGCCAGGGCGGAAAGTACAGACTCA | | |
| | ORF Start: ATG at 58 | | ORF Stop: TGA at 1096 |
| | SEQ ID NO:102 | 346 aa | MW at 38718.0 kD |

| | |
|---|---|
| NOV29b, | MESTLGAGIVIAEALQNQLAWLENVWLWITFLGDPKILFLFYFPAAYYASRRVGIAVL |
| CG59245-01 Protein Sequence | |
| | WISLITEWLNLIFKWFLFGDRPFWWVHESGYYSQAPAQVHQFPSSCETGPGSPSGHCM |
| | ITGAALWPIMTALSSQVATRARSRWVRVMPSLAYCTFLLAVGLSRIFILAHFPHQVLA |
| | GLITGAVLGWLMTPRVPMERELSFYGLTALALMLGTSLIYWTLFTLGLDLSWSISLAF |
| | KWCERPEWIHVDSRPFASLSRDSGAALGLGIALHSPCYAQVRRAQLGNGQKIACLVLA |
| | MGLLGPLDWLGHPPQISLFYIFNFLKYTLWPCPVLALVPWAVHMFSAQEAPPIHSS |

Sequence comparison of the above protein sequences yields the following sequence relationships shown in Table 29B.

TABLE 29B
Comparison of NOV29a against NOV29b.

| | NOV29a | Identities/ |
|---|---|---|
| | Residues/ | Similarities for |
| Protein | Match | the Matched |
| Sequence | Residues | Region |
| NOV29b | 1 . . . 337 | 335/347 (96%) |
| | 1 . . . 346 | 335/347 (96%) |

Further analysis of the NOV29a protein yielded the following properties shown in Table 29C.

TABLE 29C
Protein Sequence Properties NOV29a

| | |
|---|---|
| PSort | 0.6850 probability located in endoplasmic reticulum |
| analysis: | (membrane); 0.6400 probability located in plasma |
| | membrane; 0.4600 probability located in Golgi body; |
| | 0.1000 probability located in endoplasmic reticulum (lumen) |
| SignalP | Likely cleavage site between residues 41 and 42 |
| analysis: | |

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM79500 | Human protein SEQ ID NO 3146 - | 1 . . . 337 | 336/347 (96%) | 0.0 |
| | Homo sapiens, 382 aa. | 37 . . . 382 | 336/347 (96%) | |
| | [WO200157190-A2, 9 AUG | | | |
| | 2001] | | | |
| AAB42637 | Human ORFX ORF2401 | 1 . . . 337 | 328/348 (94%) | 0.0 |
| | polypeptide sequence SEQ ID | 31 . . . 377 | 328/348 (94%) | |
| | NO:4802 - Homo sapiens, 377 aa. | | | |
| | [WO200058473-A2, 5 OCT | | | |
| | 2000] | | | |
| AAB85355 | Human phosphatase (PP) (clone ID | 1 . . . 305 | 297/315 (94%) | e−174 |
| | 1269556CD1) - Homo sapiens, 385 | 1 . . . 314 | 298/315 (94%) | |
| | aa. [WO200153469-A2, 26 JUL | | | |
| | 2001] | | | |
| AAM78516 | Human protein SEQ ID NO 1178 - | 1 . . . 337 | 266/341 (78%) | e−146 |
| | Homo sapiens, 404 aa. | 125 . . . 404 | 272/341 (79%) | |
| | [WO200157190-A2, 9 AUG | | | |
| | 2001] | | | |
| AAB25679 | Human secreted protein sequence | 198 . . . 337 | 140/140 (100%) | 6e−81 |
| | encoded by gene 15 SEQ ID | 1 . . . 140 | 140/140 (100%) | |
| | NO:68 - Homo sapiens, 141 aa. | | | |
| | [WO200043495-A2, 27 JUL 2000] | | | |

In a BLAST search of public sequence databases, the NOV29a protein was found to have homology to the proteins shown in the BLASTP data in Table 29E.

TABLE 29E
Public BLASTP Results for NOV29a

| | | | Identities/ | |
|---|---|---|---|---|
| | | NOV29a | Similarities | |
| Protein | | Residues/ | for the | |
| Accession | | Match | Matched | Expect |
| Number | Protein/Organism/Length | Residues | Portion | Value |
| AAH21574 | HYPOTHETICAL 38.7 KDA | 1 . . . 337 | 336/347 (96%) | 0.0 |
| | PROTEIN - Homo sapiens | 1 . . . 346 | 336/347 (96%) | |
| | (Human), 346 aa. | | | |
| Q9BUM1 | HYPOTHETICAL 40.1 KDA | 1 . . . 337 | 336/347 (96%) | 0.0 |
| | PROTEIN - Homo sapiens | 15 . . . 360 | 336/347 (96%) | |
| | (Human), 360 aa (fragment). | | | |
| O42153 | Glucose-6-phosphatase (EC | 8 . . . 323 | 127/333 (38%) | 1e−59 |
| | 3.1.3.9) (G6Pase) (G-6-Pase) - | 8 . . . 339 | 184/333 (55%) | |
| | Haplochromis nubilus, 352 aa. | | | |
| Q98UF8 | GLUCOSE-6-PHOSPHATASE - | 8 . . . 323 | 123/333 (36%) | 2e−57 |
| | Sparus aurata (Gilthead sea | 8 . . . 337 | 185/333 (54%) | |
| | bream), 350 aa. | | | |
| Q9Z186 | GLUCOSE-6-PHOSPHATASE - | 7 . . . 325 | 128/343 (37%) | 5e−56 |
| | Mus musculus (Mouse), 355 aa. | 7 . . . 345 | 188/343 (54%) | |

PFam analysis predicts that the NOV29a protein contains the domains shown in the Table 29F.

TABLE 29F
Domain Analysis of NOV29a

| | | Identities/ | |
|---|---|---|---|
| | | Similarities | |
| Pfam | NOV29a | for the | Expect |
| Domain | Match Region | Matched Region | Value |
| PAP2: domain 1 of 1 | 51 . . . 190 | 38/175 (22%) | 0.00037 |
| | | 95/175 (54%) | |

### Example 30

The NOV30 clone was analyzed, and the nucleotide and predicted polypeptide sequences are shown in Table 30A.

TABLE 30A
NOV39 Sequence Analysis

| | |
|---|---|
| SEQ ID NO:103 | 1624 bp |

| NOV30a, | ATGGAACTGAAGGCCGAGGAGGAGGAGGTGGGTGGCGTCCAGCCGGTGGACTTGGTGG | | |
|---|---|---|---|
| CG59241-01 DNA Sequence | | | |
| | CCTTTGCCAACAGCTGCACCCTCCATGGCACCAACCACATTTTTGTGGAGGGGGGTCC | | |
| | AGGGCCAAGGCAGGTGCTGTGGGCGGTGGCCTTTGTCCTGGCACTGGGTGCCTTCCTG | | |
| | TGCCAGGTAGGGGACCGCGTTGCTTATTACCTCAGCTACCCACACGTGACCCTTCTAA | | |
| | ACGAAGTGGCCACCACGGAGCTGGCCTTCCCGGCAGTCACCCTCTGCAACACTAATGC | | |
| | TGTGCGGCTGTCCCAGCTCAGCTACCCTGACTTGCTTTATTTGGCCCCCATGCTGGGA | | |
| | CTGGATGAAAGTGATGACCCCGGGGTGCCCCTCGCTCCACCGGGCCCTGAGGCCTTCT | | |
| | CTGGGGAGCCCTTTAACCTGCACCGCTTCTACAATCGCTCCTGCCACCGGCTGGAGGA | | |
| | CATGCTGCTCTATTGCTCCTACCAAGGGGGACCCTGCGGCCCTCACAACTTCTCAGTG | | |
| | GTGTTCACACGCTATGGAAAGTGCTACACGTTCAACTCGGGCCGAGATGGGCGGCCGC | | |
| | GGCTGAAGACCATGAAGGGTGGGACGGGCAATGGGCTGGAAATCATGCTGGACATCCA | | |
| | GCAGGACGAGTACCTGCCTGTGTGGGGGGAGACTGACGAGACGTCCTTCGAAGCAGGC | | |
| | ATCAAAGTGCAGATCCATAGTCAGGATGAACCTCCTTTCATCGACCAGCTGGGCTTTG | | |
| | GCGTGGCCCCAGGCTTCCAGACCTTTGTGGCCTGCCAGGAGCAGCGGATCTACCTGCC | | |
| | CCCACCCTGGGGCACCTGCAAAGCTGTTACCATGGACTCGGATTTCTTCGACTCCTAC | | |
| | AGCATCACTGCCTGCCGCATCGACTGTGAGACGCGCTACCTGGTGGAGAACTGCAACT | | |
| | GCCGCATGGTGCACATGCCAGGTGATGCCCCATACTGTACTCCAGAGCAGTACAAGGA | | |
| | GTGTGCAGATCCTGCTCTGGACTTCCTGGTGGAGAAGGACCAGGAGTACTGCGTGTGT | | |
| | GAAATGCCTTGCAACCTGACCCGCTATGGCAAAGAGCTGTCCATGGTCAAGATCCCCA | | |
| | GCAAAGCCTCAGCCAAGTACCTGGCCAAGAAGTTCAACAAATCTGAGCAATACATAGG | | |
| | GGAGAACATCCTGGTGCTGGACATTTTCTTTGAAGTCCTCAACTATGAGACCATTGAA | | |
| | CAGAAGAAGGCCTATGAGATTGCAGGGCTCCTGGGTGACATCGGGGGCCAGATGGGGC | | |
| | TGTTCATCGGGGCCAGCATCCTCACGGTGCTGGAGCTCTTTGACTACGCCTACGAGGT | | |
| | AGTCATTAAGCACAAGCTGTGCCGACGAGGAAAATGCCAGAAGGAGGCCAAAAGGAGC | | |
| | AGTGCGGACAAGGGCGTGGCCCTCAGCCTGGACGACGTCAAAAGACACAACCCGTGCG | | |
| | AGAGCCTTCGGGGCCACCCTGCCGGGATGACATACGCTGCCAACATCCTACCTCACCA | | |
| | TCCGGCCCGAGGCACGTTCGAGGACTTTACCTGCTGAGCCCCGCAGGCCGCTGAACCA | | |
| | AAGGCCTAGATGGGGAGGACTAGGAGAGCGAGGGGGCCCCCAGCTGCCTCCTCACATC | | |
| | ORF Start: ATG at 1 | | ORF Stop: TGA at 1543 |
| | SEQ ID NO:104 | 514 aa | MW at 57221.7 kD |

| | |
|---|---|
| NOV3a, | MELKAEEEEVGGVQPVDLVAFANSCTLHGTNHIFVEGGPGPRQVLWAVAFVLALGAFL |
| CG59241-01 Protein Sequence | |
| | CQVGDRVAYYLSYPHVTLLNEVATTELAFPAVTLCNTNAVRLSQLSYPDLLYLAPMLG |
| | LDESDDPGVPLAPPGPEAFSGEPFNLHRFYNRSCHRLEDMLLYCSYQGGPCGPHNFSV |
| | VFTRYGKCYTFNSGRDGRPRLKTMKGGTGNGLEIMLDIQQDEYLPVWGETDETSFEAG |
| | IKVQIHSQDEPPFIDQLGFGVAPGFQTFVACQEQRIYLPPPWGTCKAVTMDSDFFDSY |
| | SITACRIDCETRYLVENCNCRMVHMPGDAPTCTPEQYKECADPALDFLVEKDQEYCVC |
| | EMPCNLTRYGKELSMVKIPSKASAKYLAKKFNKSEQYIGENILVLDIFFEVLNYETIE |
| | QKKAYEIAGLLGDIGGQMGLFIGASILTVLELFDYAYEVVIKHKLCRRGKCQKEAKRS |
| | SADKGVALSLDDVKRHNPCESLRGHPAGMTYAANILPHHPARGTFEDFTC |

Further analysis of the NOV30a protein yielded the following properties shown in Table 30B.

TABLE 30B
Protein Sequence Properties NOV30a

| | |
|---|---|
| PSort | 0.7900 probability located in plasma membrane; 0.3000 |
| analysis: | probability located in Golgi body; 0.2000 probability |
| | located in endoplasmic reticulum (membrane); |
| | 0.1000 probability located in mitochondrial inner membrane |
| SignalP | Likely cleavage site between residues 60 and 61 |
| analysis: | |

A search of the NOV30a protein against the Geneseq database, a proprietary database that contains sequences published in patents and patent publication, yielded several homologous proteins shown in Table 30C.

TABLE 30C
Geneseq Results for NOV30a

| | | NOV30a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY69178 | A rat acid-sensitive cationic | 1 . . . 514 | 488/515 (94%) | 0.0 |
| | channel 1B (rASIC1B) - Rattus sp, | 47 . . . 559 | 497/515 (95%) | |
| | 559 aa. [WO200008149-A2, 17 | | | |
| | FEB 2000] | | | |
| AAY03186 | Rat Acid sensitive ion channel | 1 . . . 514 | 488/515 (94%) | 0.0 |
| | protein sequence - Rattus sp, 513 | 1 . . . 513 | 498/515 (95%) | |
| | aa. [WO9911784-A1, 11 MAR | | | |
| | 1999] | | | |
| AAW68507 | Rat acid sensing ionic channel 1B - | 1 . . . 514 | 488/515 (94%) | 0.0 |
| | Rattus sp, 559 aa. [WO9835034- | 47 . . . 559 | 497/515 (95%) | |
| | A1, 13 AUG 1998] | | | |
| AAY69175 | A rat acid-sensitive cationic | 1 . . . 514 | 416/527 (78%) | 0.0 |
| | 526 aa. [WO200008149-A2, 17 | 1 . . . 526 | 445/527 (83%) | |
| | FEB 2000] | | | |
| AAY03188 | Rat Acid sensitive ion channel | 1 . . . 514 | 416/527 (78%) | 0.0 |
| | alpha protein sequence - Rattus sp, | 1 . . . 526 | 445/527 (83%) | |
| | 526 aa. [WO9911784-A1, 11 | | | |
| | MAR 1999] | | | |

In a BLAST search of public sequence databases, the NOV30a protein was found to have homology to the proteins shown in the BLASTP data in Table 30D.

TABLE 30D
Public BLASTP Results for NOV30a

| | | | Identities/ | |
|---|---|---|---|---|
| | | NOV30a | Similarities | |
| Protein | | Residues/ | for the | |
| Accession | | Match | Matched | Expect |
| Number | Protein/Organism/Length | Residues | Portion | Value |
| Q91YB8 | ION CHANNEL - Rattus norvegicus | 1 . . . 514 | 489/515 (94%) | 0.0 |
| | (Rat), 559 aa. | 47 . . . 559 | 498/515 (95%) | |
| O88762 | ASIC-BETA - Rattus norvegicus | 1 . . . 514 | 488/515 (94%) | 0.0 |
| | (Rat), 513 aa. | 1 . . . 513 | 498/515 (95%) | |
| P55926 | Amiloride-sensitive brain sodium | 1 . . . 514 | 416/527 (78%) | 0.0 |
| | channel BNaC2 (Amiloride-sensitive | 1 . . . 526 | 445/527 (83%) | |
| | cation channel neuronal 2) (Proton | | | |
| | gated cation channel ASIC1) - Rattus | | | |
| | norvegicus (Rat), 526 aa. | | | |
| P78348 | Amiloride-sensitive brain sodium | 1 . . . 514 | 421/575 (73%) | 0.0 |
| | channel BNaC2 (Amiloride-sensitive | 1 . . . 574 | 447/575 (77%) | |
| | cation channel neuronal 2) - Homo | | | |
| | sapiens (Human), 574 aa. | | | |
| Q99NA1 | PROTON-GATED CATION | 175 . . . 514 | 334/341 (97%) | 0.0 |
| | CHANNEL SUBUNIT ASIC- | 86 . . . 425 | 337/341 (97%) | |
| | BETA2 - Rattus norvegicus (Rat), | | | |
| | 425 aa. | | | |

PFam analysis predicts that the NOV30a protein contains the domains shown in the Table 30E.

TABLE 30E
Domain Analysis of NOV30a

| | | Identities/ | | |
|---|---|---|---|---|
| | | Similarities | | |
| Pfam | NOV30a | for the | | Expect |
| Domain | Match Region | Matched Region | | Value |
| ASC: domain 1 of 2 | 21 . . . 118 | 34/106 | (32%) | 1.6e−29 |
| | | 79/106 | (75%) | |
| ASC: domain 2 of 2 | 145 . . . 442 | 133/351 | (38%) | 2.1e−139 |
| | | 281/351 | (80%) | |

### Example 31

The NOV31 clone was analyzed, and the nucleotide and predicted polypeptide sequences are shown in Table 31A.

TABLE 31A
NOV31 Sequence Analysis

| | SEQ ID NO:105 | 1949 bp | |
|---|---|---|---|
| NOV31a, | TGCCTGGCTATGGCCCGACTGCTCAGGTCTGCAACCTGGGAGCTGTTCCCCTGGAGGG | | |
| CG58602-01 DNA Sequence | | | |
| | GCTACTGCTCCCAGTCCCTGCAGGGAGAGCTCTGCAGGGACTTCGTAGAGGCTCTGAA | | |
| | GGCCGTGGTGGGCGGCTCCCACGTGTCCACTGCCGCGGTGGTCCGAGAGCAGCACGGG | | |
| | CGCGATGAGTCGGTGCACAGGTGCGAACCTCCTGATGCTGTGGTGTGGCCCCAGAACG | | |
| | TGGAGCAGGTCAGCCGGCTGGCAGCCCTGTGCTATCGCCAAGGTGTGCCCATCATCCC | | |
| | ATTCGGCACCGGCACCGGGCTTGAGGGTGGCGTCTGTGCTGTGCAGGGCGGCGTCTGC | | |
| | GTTAACCTGACGCATATGGACCGAATCCTGGAGCTGAACCAGGAGGACTTCTCTGTGG | | |
| | TGGTGGAGCCAGGTGTCACCCGCAAAGCCCTCAACGCCAACCTGCGGGACAGCGGCCT | | |
| | CTGGTTTCCTCCAGACCCAGGCGCGGACGCCTCTCTCTGTGGCATGGCGGCCACCGGG | | |
| | GCGTCGGGGACCAACGCGGTCCGCTACGGCACCATGCGGGACAACGTGCTCAACCTGG | | |
| | AGGTGGTGCTGCCCGACGGGCGGCTGCTGCACACGGCGGGCCGAGGCCTCATCACAGA | | |
| | TTCCACTGCTGCATTCCCCCACATCAGCCCCACTGAGTGCTTTTCCCAGGGGCCAGGG | | |
| | CCTCATGTCAATTCTCCTCACCCTGCCCCTGAGGCCACAGTGGCCGCCACGTGTGCGT | | |
| | TCCCCAGTGTCCAGGCTGCTGTGGACAGCACTGTACACATCCTCCAGGCTGCAGTGCC | | |
| | CGTAGCCCGCATTGAGTTCCTGGATGAAGTCATGATGGATGCCTGCAACAGGTACAGC | | |
| | AAGCTGAATTGCTTAGTGGCGCCCACACTCTTCCTGGAGTTCCATGGCTCCCAGCAGG | | |
| | CACTGGAGGAGCAGCTGCAGCGCACAGAGGAGATAGTCCAGCAGAACGGAGCCTCTGA | | |
| | CTTCTCCTGGGCCAAGGAGGCCGAGGAGCGCAGCCGGCTTTGGACAGCACGGCACAAT | | |
| | GCCTGGTACGCAGCCCTGGCCACGCGGCCAGGCTGCAAGGGCTACTCCACGGATGTGT | | |
| | GTGTGCCCATCTCCCGGCTGCCGGAGATCGTGGTGCAGACCAAGGAGGATCTGAATGC | | |
| | CTCAGGACTCACAGGAAGCATTGTCGGGCATGTGGGTGACGGCAACTTCCACTGCATC | | |
| | CTGCTGGTCAACCCTGATGACGCCGAGGAACTGGGCAGGGTCAAGGCTTTTGCAGAAC | | |
| | AGCTGGGCAGGCGGGCACTGGCTCTCCACGGAACGTGCACCGGGGGAGCATGGCATGG | | |
| | AATGGGCAAGCGGCAGCTGCTGCAGGAGGAGGTGGGCGCCGTGGGCGTGGAGACCATG | | |
| | CGGCAGCTCAAGGCCGTGCTAGACCCCCAAGGCCTCATGAATCCAGGCAAAGTGCTGT | | |
| | GAAGGGGGTCTGAGCACTTAGCCCACAAGTTCCCTGACTACGGAGCCGGTTCTGGAAC | | |
| | TTTTCTTCATGCCACGGCCCCTGCAAGGAAATAGATGCTGAGGCAGTCTTCCTGCCAG | | |
| | CGAGCCCACTGTATCTGGGCCCAAGGCCAGAGGGCCCAGAGAGAAGCCTGAGCACCGT | | |
| | GTTACCTCCCTGGCCCTCTGGCTGGCCCCAGGAGCCTTTGGTTCAGTAAACGACCCAG | | |
| | GGTGGTTCCCAGCAAAGCTGCTTCCTCTCTGCTCCTACGCATCCTGTCCTGGCGGGAA | | |
| | GAGAGCGTCTGGGTCCATTCAAGACTCTGATGACACCCCTCCCCGAGGCCTCCCACTG | | |
| | CCGGGGTCCCAGGACCCTTCCCCCTTCACCTGGTGACAGGAACACTCCTTTCCTGGTA | | |
| | TGGAACGTGAGCTCCCGTGACATGATGATAGGTCTTCTCCTTGGGGCCTCCCCCAATA | | |
| | AATCTGTAATAAACCTGAAACCCACCTACAGCTAA | | |
| | ORF Start: ATG at 10 | | ORF Stop: TGA at 1450 |
| | SEQ ID NO:106 | 480 aa | MW at 51629.1 kD |

| | |
|---|---|
| NOV31a, | MARLLRSATWELFPWRGYCSQSLQGELCRDFVEALKAVVGGSHVSTAAVVREQHGRDE |
| CG58602-01 Protein Sequence | |
| | SVHRCEPPDAVVWPQNVEQVSRLAALCYRQGVPIIPFGTGTGLEGGVCAVQGGVCVNL |
| | THMDRILELNQEDFSVVVEPGVTRKALNAHLRDSGLWFPPDPGADASLCGMAATGASG |
| | TNAVRYGTMRDNVLNLEVVLPDGRLLHTAGRGLITDSTAAFPHISPTECFSQGPGPHV |
| | NSPHPAPEATVAATCAFPSVQAAVDSTVHILQAAVPVARIEFLDEVMMDACNRYSKLN |
| | CLVAPTLFLEFHGSQQALEEQLQRTEEIVQQNGASDFSWAKEAEERSRLWTARHNAWY |
| | AALATRPGCKGYSTDVCVPISRLPEIVVQTKEDLNASGLTGSIVGHVGDGNFHCILLV |
| | NPDDAEELGRVKAFAEQLGRRALALHGTCTGEHGIGMGKRQLLQEEVGAVGVETMRQL |
| | KAVLDPQGLMNPGKVL |

Further analysis of the NOV31a protein yielded the following properties shown in Table 31B.

TABLE 31B
Protein Sequence Properties NOV31a

| | |
|---|---|
| PSort | 0.6574 probability located in mitochondrial matrix space; |
| analysis: | 0.3502 probability located in mitochondrial inner membrane; |
| | 0.3502 probability located in mitochondrial intermembrane |
| | space; 0.3502 probability located in mitochondrial outer |
| | membrane |
| SignalP | Likely cleavage site between residues 20 and 21 |
| analysis: | |

A search of the NOV31a protein against the Geneseq database, a proprietary database that contains sequences published in patents and patent publication, yielded several homologous proteins shown in Table 31C.

TABLE 31C
Geneseq Results for NOV31a

| | | NOV31a | Identities/ | | |
|---|---|---|---|---|---|
| | | Residues/ | Similarities for | | |
| Geneseq | Protein/Organism/Length | Match | the Matched | | Expect |
| Identifier | [Patent #, Date] | Residues | Region | | Value |
| ABB10446 | Human cDNA SEQ ID NO: 754 - | 1 . . . 96 | 91/96 | (94%) | 8e−49 |
| | Homo sapiens, 115 aa. | 15 . . . 110 | 92/96 | (95%) | |
| | [WO200154474-A2, 2 AUG 2001] | | | | |
| AAE09597 | Human gene 5 encoded novel | 1 . . . 96 | 91/96 | (94%) | 8e−49 |
| | protein HDPMT22, SEQ ID NO:33 - | 15 . . . 110 | 92/96 | (95%) | |
| | Homo sapiens, 115 aa. | | | | |
| | [WO200155311-A2, 2 AUG 2001] | | | | |
| AAM52368 | GIP12-C4 protein - Arabidopsis | 66 . . . 203 | 69/138 | (50%) | 9e−34 |
| | thaliana, 159 aa. [FR2806095-A1, | 3 . . . 140 | 98/138 | (71%) | |
| | 14 SEP 2001] | | | | |
| AAG92286 | C glutamicum protein fragment SEQ | 46 . . . 477 | 108/486 | (22%) | 2e−22 |
| | ID NO: 6040 - Corynebacterium | 25 . . . 502 | 186/486 | (38%) | |
| | glutamicum, 948 aa. [EP1108790- | | | | |
| | A2, 20 JUN 2001] | | | | |
| AAB79309 | Corynebacterium glutamicum SMP | 46 . . . 477 | 108/486 | (22%) | 2e−22 |
| | protein sequence SEQ ID NO:134 - | 22 . . . 499 | 186/486 | (38%) | |
| | Corynebacterium glutamicum, 945 | | | | |
| | aa. [WO200100844-A2, 4 JAN | | | | |
| | 2001] | | | | |

In a BLAST search of public sequence databases, the NOV31a protein was found to have homology to the proteins shown in the BLASTP data in Table 31D.

TABLE 31D
Public BLASTP Results for NOV31a

| | | | Identities/ | |
|---|---|---|---|---|
| | | NOV31a | Similarities | |
| Protein | | Residues/ | for the | |
| Accession | | Match | Matched | Expect |
| Number | Protein/Organism/Length | Residues | Portion | Value |
| Q9D635 | 4733401P21RIK PROTEIN - Mus | 1 . . . 480 | 394/483 (81%) | 0.0 |
| | musculus (Mouse), 481 aa. | 1 . . . 481 | 423/483 (87%) | |
| Q19965 | F32D8.4 PROTEIN - Caenorhabditis | 20 . . . 480 | 221/466 (47%) | e−121 |
| | elegans, 912 aa. | 445 . . . 909 | 307/466 (65%) | |
| CAD16371 | PUTATIVE D-LACTATE | 32 . . . 479 | 226/454 (49%) | e−119 |
| | DEHYDROGENASE | 20 . . . 469 | 300/454 (65%) | |
| | (CYTOCHROME) | | | |
| | OXIDOREDUCTASE PROTEIN | | | |
| | (EC 1.1.2.4) - Ralstonia | | | |
| | solanacearum (Pseudomonas | | | |
| | solanacearum), 472 aa. | | | |
| A89201 | protein F32D8.4 [imported] - | 30 . . . 480 | 214/469 (45%) | e−115 |
| | Caenorhabditis elegans, 870 aa. | 399 . . . 867 | 296/469 (62%) | |
| AAL51780 | D-LACTATE DEHYDROGENASE | 41 . . . 480 | 209/444 (47%) | e−114 |
| | (CYTOCHROME) (EC 1.1.2.4) - | 28 . . . 467 | 286/444 (64%) | |
| | Brucella melitensis, 468 aa. | | | |

PFam analysis predicts that the NOV31a protein contains the domains shown in the Table 31E.

TABLE 31E
Domain Analysis of NOV31a

| | | Identities/ | | |
|---|---|---|---|---|
| | | Similarities | | |
| Pfam | NOV31a | for the | | Expect |
| Domain | Match Region | Matched Region | | Value |
| FAD_binding_4: | 33 . . . 214 | 70/208 | (34%) | 3.7e−56 |
| domain 1 of 1 | | 154/208 | (74%) | |
| FAD-oxidase_C: | 206 . . . 479 | 91/307 | (30%) | 1.3e−58 |
| domain 1 of 1 | | 210/307 | (68%) | |

### Example 32

The NOV32 clone was analyzed, and the nucleotide and predicted polypeptide sequences are shown in Table 32A.

TABLE 32A
NOV32 Sequence Analysis

| | SEQ ID NO:107 | 698 bp | |
|---|---|---|---|
| NOV32a, | CTCCTTCCTGTGCTCTTTATATGGACCAACAACTTCTCGTCCTTGGGGTCTCTGTGCA | | |
| CG58468-01 DNA Sequence | | | |
| | AATATCAATTTTTTCACATTATCTTTTCTCCACAGACATGAGAGGGAAGGCATTTATT | | |
| | TTCCCTCAAGAATCAGCTACAGTCTATGTGTCCCTGATCCCCAAGGTGAAGAAGCCCC | | |
| | TGAAGAACTTCAAGCTTTGCCTGAAAACCTTCACAGACTTCACCTGCCCTTATAGCCT | | |
| | CTTCTACAGCACTCCGTCCCAGGACAATGAGCTGCTTCTCCTTGTCAACAAAATGGGA | | |
| | ATGTATCTGCTGCACATTGGAAATGCTGCGGTCACTTTCAATGGCCCCACCCCCTGCC | | |
| | CTCGATCTCCTTATGCTTCGACCCATGTCAATGTGAGCTGGGAGTCTGCCTCTGGAAT | | |
| | TGCTACACTCTGGGCAAATGGGAAGCTGGTGGGGAGGAAGGGTGTGTGGAAGGGGTAC | | |
| | TCTGTGGGAGAAGAGGCTAAGATCATCCTGGGACAAGAGCAGGATTCCTTTGGGGGAC | | |
| | ATTTTGATGAAAATCAATCCTTTGTTGGGGTGATATGGGATGTGTTTTTGTGGGATCA | | |
| | TGTGCTCCCTCCAAAGGAGATGTGTGACTCCTGTTACAGCGGCAGCCTCCTGAATCGG | | |
| | CATACCCTGACTTATGAAGATAATGGCTATGTGGTAACTAAGCCCAAGGTGTGGGCTT | | |
| | AA | | |
| | ORF Start: ATG at 21 | | ORF Stop: TAA at 696 |
| | SEQ ID NO:108 | 225 aa | MW at 25265.8 kD |

| | |
|---|---|
| NOV32a, | MDQQLLVLGVSVQISIFSHYLFSTDMRGKAFIFPQESATVYVSLIPKVKKPLKNFKLC |
| CG58468-01 Protein Sequence | |
| | LKTFTDFTCPYSLFYSTRSQDNELLLLVNKMGMYLLHIGNAAVTFNGPTPCPRSPYAS |
| | THVNVSWESASGIATLWANGKLVGRKGVWKGYSVGEEAKIILGQEQDSFGGHFDENQS |
| | FVGVIWDVFLWDHVLPPKEMCDSCYSGSLLNRHTLTYEDNGYVVTKPKVWA |

Further analysis of the NOV32a protein yielded the following properties shown in Table 32B.

TABLE 32B
Protein Sequence Properties NOV32a

| | |
|---|---|
| PSort | 0.5500 probability located in endoplasmic reticulum |
| analysis: | (membrane); 0.3200 probability located in microbody |
| | (peroxisome); 0.2368 probability located in lysosome |
| | (lumen); 0.1000 probability located in endoplasmic |
| | reticulum (lumen) |
| SignalP | No Known Signal Sequence Predicted |
| analysis: | |

A search of the NOV32a protein against the Geneseq database, a proprietary database that contains sequences published in patents and patent publication, yielded several homologous proteins shown in Table 32C.

TABLE 32C
Geneseq Results for NOV32a

| | | NOV32a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR74763 | Sermun amyloid P component, | 24 . . . 224 | 98/207 (47%) | 4e−48 |
| | promoter sapm-Homo sapiens, | 2 . . . 203 | 136/207 (65%) | |
| | 204 aa. [WO9505394-A, | | | |
| | 23 Feb. 1995] | | | |
| AAR29923 | SAP-Homo sapiens, 223 aa. | 7 . . . 224 | 101/224 (45%) | 3e−47 |
| | [WO9221364-A, 10 DEC. 1992] | 5 . . . 222 | 143/224 (63%) | |
| AAR29922 | CRP-Homo sapiens, 225 aa. | 14 . . . 224 | 100/218 (45%) | 2e−43 |
| | [WO9221364-A, 10 DEC. 1992] | 11 . . . 224 | 132/218 (59%) | |
| AAR74769 | Female hamster protein, 1 fhp- | 24 . . . 222 | 95/206 (46%) | 6e−43 |
| | Cricetus cricetus, 210 aa. | 1 . . . 199 | 132/206 (63%) | |
| | [WO9505394-A, 23 FEB. 1995] | | | |
| AAY76844 | Human C reactive protein (CRP) | 24 . . . 224 | 98/208 (47%) | 1e−42 |
| | sequence-Homo sapiens, 206 aa. | 2 . . . 205 | 128/208 (61%) | |
| | [JP2000014388-A, | | | |
| | 18 JAN. 2000] | | | |

In a BLAST search of public sequence databases, the NOV32a protein was found to have homology to the proteins shown in the BLASTP data in Table 32D.

TABLE 32D
Public BLASTP Results for NOV32a

| | | NOV32a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9D8J8 | 1810030J14RIK PROTEIN-Mus | 6 . . . 224 | 130/220 (59%) | 5e−72 |
| | musculus (Mouse), 219 aa. | 4 . . . 218 | 166/220 (75%) | |
| Q9D8V2 | 1810030J14RIK PROTEIN-Mus | 6 . . . 190 | 110/186 (59%) | 2e−58 |
| | musculus (Mouse), 200 aa. | 20 . . . 200 | 139/186 (74%) | |
| Q63913 | SERUM AMYLOID P-Cricetulus | 1 . . . 224 | 109/231 (47%) | 4e−51 |
| | migratorius (Armenian hamster), | 1 . . . 222 | 152/231 (65%) | |
| | 223 aa. | | | |
| P23680 | Serum amyloid P-component | 6 . . . 224 | 105/224 (46%) | 7e−50 |
| | precursor (SAP)-Rattus norvegicus | 4 . . . 223 | 145/224 (63%) | |
| | (Rat), 228 aa. | | | |
| P15697 | Female protein precursor (FP) | 1 . . . 222 | 108/229 (47%) | 7e−50 |
| | (Serum amyloid P-component)- | 1 . . . 220 | 151/229 (65%) | |
| | Cricetulus migratorius (Armenian | | | |
| | hamster), 231 aa. | | | |

PFam analysis predicts that the NOV32a protein contains the domains shown in the Table 32E.

TABLE 32E
Domain Analysis of NOV32a

| | | Identities/ | |
|---|---|---|---|
| Pfam | NOV32a Match | Similarities for | Expect |
| Domain | Region | the Matched Region | Value |
| pentaxin: domain 1 of | 29 . . . 221 | 103/214 (48%) | 8e−76 |
| 1 | | 156/214 (73%) | |

### Example 33

The NOV33 clone was analyzed, and the nucleotide and predicted polypeptide sequences are shown in Table 33A.

TABLE 33A
NOV33 Sequence Analysis

| | SEQ ID NO:109 | 3350 bp | |
|---|---|---|---|
| NOV33a, | TAATGAGGAGACTGAGTTTGTGGTGGCTGCTGAGCAGGGTCTGTCTGCTGTTGCCGCC | | |
| CG58183-01 DNA Sequence | | | |
| | GCCCTGCGCACTGGTGCTGGCCGGGGTGCCCAGCTCCTCCTCGCACCCGCAGCCCTGC | | |
| | CAGATCCTCAAGCGCATCGGGCACGCGGTGAGGGTGGGCGCGGTGCACTTGCAGCCCT | | |
| | GGACCACCGCCCCCCGCGCGGCCAGCCGCGCTCCGGACGACAGCCGAGCAGGAGCCCA | | |
| | GAGGGATGAGCCGGAGCCAGGGACTAGGCGGTCCCCGGCGCCCTCGCCGGGCGCACGC | | |
| | TGGTTGGGGAGCACCCTGCATGGCCGGGGGCCGCCGGGCTCCCGTAAGCCCGGGGAGG | | |
| | GCGCCAGGGCGGAGGCCCTGTGGCCACGGGACGCCCTCCTATTTGCCGTGGACAACCT | | |
| | GAACCGCGTGGAAGGGCTGCTACCCTACAACCTGTCTTTGGAAGTAGTGATGGCCATC | | |
| | GAGGCAGGCCTGGGCGATCTGCCACTTTTGCCCTTCTCCTCCCCTAGTTCGCCATGGA | | |
| | GCAGTGACCCTTTCTCCTTCCTGCAAAGTGTGTGCCATACCGTGGTGGTGCAAGGGGT | | |
| | GTCGGCGCTGCTCGCCTTCCCCCAGAGCCAGGGCGAAATGATGGAGCTCGACTTGGTC | | |
| | AGCTTAGTCCTGCACATTCCAGTGATCAGCATCGTGCGCCACGAGTTTCCACGGGAGA | | |
| | GTCAGAATCCCCTTCACCTACAACTGAGTTTAGAAAATTCATTAAGTTCTGATGCTGA | | |
| | TGTCACTGTCTCAATCCTGACCATGAACAACTGGTACAATTTTAGCTTGTTGCTGTGC | | |
| | CAGGAAGACTGGAACATCACCGACTTCCTCCTCCTTACCCAGAATAATTCCAAGTTCC | | |
| | ACCTTGGTTCTATCATCAACATCACCGCTAACCTCCCCTCCACCCAGGACCTCTTGAG | | |
| | CTTCCTACAGATCCAGCTTGAGAGTATTAAGAACAGCACACCCACAGTGGTGATGTTT | | |
| | GGCTGCGACATGGAAAGTATCCGGCGGATTTTCGAAATTACAACCCAGTTTGGGGTCA | | |
| | TGCCCCCTGAACTTCGTTGGGTGCTGGGAGATTCCCAGAATGTGGAGGAACTGAGGAC | | |
| | AGAGGGTCTGCCCTTAGGGCTCATTGCTCATGGAAAAACAACACAGTCTGTCTTTGAG | | |
| | CACTACGTACAAGATGCTATGGAGCTGGTCGCAAGAGCTGTAGCCACAGCCACCATGA | | |
| | TCCAACCAGAACTTGCTCTCATTCCCAGCACGATGAACTGCATGGAGGTGGAAACTAC | | |
| | AAATCTCACTTCAGGACAATATTTATCAAGGTTTCTAGCCAATACCACTTTCAGAGGC | | |
| | CTCAGTGGTTCCATCAGAGTAAAAGGTTCCACCATCGTCAGCTCAGAAAACAACTTTT | | |
| | TCATCTGGAATCTTCAACATCACCCCATGGGAAAGCCAATGTGGACCCGCTTGGGCAG | | |
| | CTGGCAGGGGGGAAAGATTGTCATGGACTATGGAATATGGCCAGAGCAGGCCCAGAGA | | |
| | CACAAAACCCACTTCCAACATCCAAGTAAGCTACACTTGAGAGTGGTTACCCTGATTG | | |
| | AGCATCCTTTTGTCTTCACAAGGGAGGTAGATGATGAAGGCTTGTGCCCTGCTGGCCA | | |
| | ACTCTGTCTAGACCCCATGACTAATGACTCTTCCACATTGGACAGCCTTTTTAGCAGC | | |
| | CTCCATAGCAGTAATGATACAGTGCCCATTAAATTCAAGAAGTGCTGCTATGGATATT | | |
| | GCATTGATCTGCTGGAAAAGATAGCAGAAGACATGAACTTTGACTTCGACCTCTATAT | | |
| | TGTAGGGGATGGAAAGTATGGAGCATGGAAAAATGGGCACTGGACTGGGCTAGTGGGT | | |
| | GATCTCCTGAGAGGGACTGCCCACATGGCAGTCACTTCCTTTAGCATCAATACTGCAC | | |
| | GGAGCCAGGTGATAGATTTCACCAGCCCTTTCTTCTCCACCAGCTTGGGCATCTTAGT | | |
| | GAGGACCCGAGATACAGCAGCTCCCATTGGAGCCTTCATGTGGCCACTCCACTGGACA | | |
| | ATGTGGCTGGGGATTTTTGTGGCTCTGCACATCACTGCCGTCTTCCTCACTCTGTATG | | |
| | AATGGAAGAGTCCATTTGGTTTGACTTCCAAGGCGCGAAATAGAAGTAAAGTCTTCTC | | |
| | CTTTTCTTCAGCCTTGAACATCTGTTATGCCCTCTTGTTTGGCAGAACAGTGGCCATC | | |
| | AAACCTCCAAAATGTTGGACTGGAAGGTTTCTAATGAACCTTTGGGCCATTTTCTGTA | | |
| | TGTTTTGCCTTTCCACATACACGGCAAACTTGCCTGCTGTCATGGTAGGTGAGAAGAT | | |
| | CTATGAAGAGCTTTCTGGAATACATGACCCCAAGTTACATCATCCTTCCCAAGGATTC | | |
| | CGCTTTGGAACTGTCCGAGAAAGCAGTGCTGAAGATTATGTGAGACAAAGTTTCCCAG | | |
| | AGATGCATGAATATATGAGAAGGTACAATGTTCCAGCCACCCCTGATGGAGTGGAGTA | | |
| | TCTGAAGAATGATCCAGAGAAACTAGACGCCTTCATCATGGACAAAGCCCTTCTGGAT | | |
| | TATGAAGTGTCAATAGATGCTGACTGCAAACTTCTCACTGTGGGGAAGCCATTTGCCA | | |
| | TAGAAGGTTACGGCATTGGCCTCCCACCCAACTCTCCATTGACCGCCAACATATCCGA | | |
| | GCTAATCAGTCAATACAAGTCACATGGGTTTATGGATATGCTCCATGACAAGTGGTAC | | |
| | AGGGTGGTTCCCTGTGGCAAGAGAAGTTTTGCTGTCACGGAGACTTTGCAAATGGGCA | | |
| | TCAAACACTTCTCTGGGCTCTTTGTGCTGCTGTGCATTGGATTTGGTCTGTCCATTTT | | |
| | GACCACCATTGGTGAGCACATAGTATACAGGCTGCTGCTACCACGAATCAAAAACAAA | | |
| | TCCAAGCTGCAATACTGGCTCCACACCAGCCAGAGATTACACAGAGCAATAAATACAT | | |
| | CATTTATAGAGGAAAAGCAGCAGCATTTCAAGACCAAACGTGTGGAAAAGAGATCTAA | | |
| | TGTGGGACCCCGTCAGCTTACCGTATGGAATACTTCCAATCTGAGTCATGACAACCGA | | |
| | CGGAAATACATCTTTAGTGATGAGGAAGGACAAAACCAGCTGGGCATCCGGATCCACC | | |
| | AGGACATCCCCCTCCCTCCAAGGAGAAGAGAGCTCCCTGCCTTGCGGACCACCAATGG | | |
| | GAAAGCAGACTCCCTAAATGTATCTCGGAACTCAGTGATGCAGGAACTCTCAGAGCTC | | |
| | GAGAAGCAGATTCAGGTGATCCGTCAGGAGCTGCAGCTGGCTGTGAGCAGGAAAACGG | | |
| | AGCTGGAGGAGTATCAAAGGACAAGTCGGACTTGTGAGTCCTAG | | |
| | ORF Start: ATG at 3 | | ORF Stop: TAG at 3348 |
| | SEQ ID NO:110 | 1115 aa | MW at 125453.7 kD |

| | |
|---|---|
| NOV33a, | MRRLSLWWLLSRVCLLLPPPCALVLAGVPSSSSHPQPCQILKRIGHAVRVGAVHLQPW |
| CG58183-01 Protein Sequence | |
| | TTAPRAASRAPDDSRAGAQRDEPEPGTRRSTAPSPGARWLGSTLHGRGPPGSRKPGEG |
| | ARAEALWPRDALLFAVDNLNRVEGLLPYNLSLEVVNAIEAGLGDLPLLPFSSPSSPWS |
| | SDPFSFLQSVCHTVVVQGVSALLAFPQSQGEMMELDLVSLVLHIPVISIVRHEFPRES |
| | QNPLHLQLSLENSLSSDADVTVSILTMNNWYNFSLLLCQEDWNITDFLLLTQNNSKFH |
| | LGSIINITANLPSTQDLLSFLQIQLESIKNSTPTVVMFGCDMESIRRIFEITTQFGVM |
| | PPELRWVLGDSQNVEELRTEGLPLGLIAHGKTTQSVFEHYVQDAMELVARAVATATMI |
| | QPELALIPSTMNCMEVETTNLTSGQYLSRFLANTTFRGLSGSIRVKGSTIVSSENNFF |
| | IWNLQHDPMGKPMWTRLGSWQGCKIVMDYGIWPEQAQRNKTHFQHPSKLHLRVVTLIE |
| | HPFVFTREVDDEGLCPAGQLCLDPMTNDSSTLDSLFSSLHSSNDTVPIKFKKCCYGYC |
| | IDLLEKIAEDMNFDFDLYIVGDGKYGAWKNGHWTGLVGDLLRGTAHMAVTSFSINTAR |
| | SQVIDFTSPFFSTSLGILVRTRDTAAPIGAFMWPLHWTMWLGIFVALHITAVFLTLYE |
| | WKSPFGLTSKGRNRSKVFSFSSALNICYALLFGRTVAIKPPKCWTGRFLMNLWAIFCM |
| | FCLSTYTANLAAVMVGEKIYEELSGIHDPKLNHPSQGFRFGTVRESSAEDYVRQSFPE |
| | MHEYMRRYNVPATPDGVEYLKNDPEKLDAFIMDKALLDYEVSIDADCKLLTVGKPFAI |
| | EGYGIGLPPNSPLTANISELISQYKSHGFMDMLHDKWYRVVPCGKRSFAVTETLQNGI |
| | KHFSGLFVLLCIGFGLSILTTIGEHIVYRLLLPRIKNKSKLQYWLHTSQRLHRAINTS |
| | FIEEKQQHFKTKRVEKRSNVGPRQLTVWNTSNLSHDNRRKYIFSDEEGQNQLGIRIHQ |
| | DIPLPPRRRELPALRTTNGKADSLNVSRNSVMQELSELEKQIQVIRQELQLAVSRKTE |
| | LEEYQRTSRTCES |

Further analysis of the NOV33a protein yielded the following properties shown in Table 33B.

TABLE 33B
Protein Sequence Properties NOV33a

| | |
|---|---|
| PSort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic |
| | reticulum (lumen) |
| SignalP | Likely cleavage site between residues 34 and 35 |
| analysis: | |

A search of the NOV33a protein against the Geneseq database, a proprietary database that contains sequences published in patents and patent publication, yielded several homologous proteins shown in Table 33C.

TABLE 33C
Geneseq Results for NOV33a

| | | NOV33a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU02199 | Human glutamate receptor-like | 95 . . . 1103 | 508/1047 (48%) | 0.0 |
| | protein, MEM4-Homo sapiens, | 6 . . . 1007 | 680/1047 (64%) | |
| | 1043 aa. [WO200144473-A2, | | | |
| | 21 JUN. 2001] | | | |
| AAB42494 | Human ORFX ORF2258 | 95 . . . 985 | 484/912 (53%) | 0.0 |
| | polypeptide sequence SEQ ID | 6 . . . 885 | 635/912 (69%) | |
| | NO: 4516-Homo sapiens, | | | |
| | 901 aa. [WO200058473-A2, | | | |
| | 5 OCT. 2000] | | | |
| AAU02198 | Human glutamate receptor-like | 532 . . . 1103 | 361/579 (62%) | 0.0 |
| | protein, MEM3-Homo sapiens, | 362 . . . 935 | 448/579 (77%) | |
| | 971 aa. [WO200144473-A2, | | | |
| | 21 JUN. 2001] | | | |
| AAU02197 | Human glutamate receptor-like | 532 . . . 1103 | 352/579 (60%) | 0.0 |
| | protein, MEM2-Homo sapiens, | 362 . . . 929 | 437/579 (74%) | |
| | 965 aa. [WO200144473-A2, | | | |
| | 21 JUN. 2001] | | | |
| AAR44192 | Rat NMDA receptor subunit, | 175 . . . 1023 | 245/873 (28%) | 2e−83 |
| | NR2A-Rattus rattus, 1464 aa. | 77 . . . 911 | 425/873 (48%) | |
| | [DE4216321-A, 18-NOV. 1993] | | | |

In a BLAST search of public sequence databases, the NOV33a protein was found to have homology to the proteins shown in the BLASTP data in Table 33D.

TABLE 33D
Public BLASTP Results for NOV33a

| | | NOV33a | | |
|---|---|---|---|---|
| Protein | | Residues/ | Identities/ | |
| Accession | | Match | Similarities for | Expect |
| Number | Protein/Organism/Length | Residues | the Matched Portion | Value |
| AAL40734 | N-METHYL-D-ASPARTATE | 1 . . . 1115 | 1110/1115 (99%) | 0.0 |
| | RECEPTOR 3A-Homo sapiens | 1 . . . 1115 | 1112/1115 (99%) | |
| | (Human), 1115 aa. | | | |
| Q62800 | IONOTROPIC GLUTAMATE | 1 . . . 1115 | 1032/1115 (92%) | 0.0 |
| | RECEPTOR-Rattus norvegicus | 1 . . . 1115 | 1083/1115 (96%) | |
| | (Rat), 1115 aa. | | | |
| Q9R1M7 | N-METHYL-D-ASPARTATE | 1 . . . 1115 | 1032/1135 (90%) | 0.0 |
| | RECEPTOR SPLICE | 1 . . . 1135 | 1083/1135 (94%) | |
| | VARIANT NR3A-2-Rattus | | | |
| | norvegicus (Rat), 1135 aa. | | | |
| CAC69380 | SEQUENCE 7 FROM PATENT | 95 . . . 1103 | 508/1047 (48%) | 0.0 |
| | WO0144473-Homo sapiens | 6 . . . 1007 | 680/1047 (64%) | |
| | (Human), 1043 aa. | | | |
| Q91ZU9 | NMDA-TYPE GLUTAMATE | 112 . . . 1103 | 510/1001 (50%) | 0.0 |
| | RECEPTOR SUBUNIT NR3B | 34 . . . 980 | 669/1001 (65%) | |
| | PRECURSOR-Mus musculus | | | |
| | (Mouse), 1003 aa. | | | |

PFam analysis predicts that the NOV33a protein contains the domains shown in the Table 33E.

TABLE 33E
Domain Analysis of NOV33a

| | | Identities/ | |
|---|---|---|---|
| Pfam | NOV33a Match | Similarities for | Expect |
| Domain | Region | the Matched Region | Value |
| lig_chan: domain 1 of | 674 . . . 952 | 81/323 (25%) | 4e−95 |
| 1 | | 232/323 (72%) | |

### Example 34

The NOV34 clone was analyzed, and the nucleotide and predicted polypeptide sequences are shown in Table 34A.

TABLE 34A
NOV34 Sequence Analysis

| | SEQ ID NO:111 | 1253 bp | |
|---|---|---|---|
| NOV34a, | CCCAGCGCCATGGGGGAGTGGGCGTTCCTGGGCTCGCTGCTGGACGCCGTGCAGCTGC | | |
| CG59315-01 DNA Sequence | | | |
| | AGTCGCCGCTCGTGGGCCGCCTCTGGCTGGTGGTCATGCTGATCTTCCGCATCCTGGT | | |
| | GCTGGCCACGGTGGGCGGCGCCGTGTTCGAGGACGAGCAAGAGGAGTTCGTGTGCAAC | | |
| | ACGCTGCAGCCGGGCTGTCGCCAGACCTGCTACGACCGCGCCTTCCCGGTCTCCCACT | | |
| | ACCGCTTCTGGCTCTTCCACATCCTGCTGCTCTCCGCGCCCCCGGTGCTGTTCGTCGT | | |
| | CTACTCCATGCACCGGGCAGGCAAGGAGGCGGGCGGCGCTGAGGCGGCGGCGCAGTGC | | |
| | GCCCCCGGACTGCCCGAGGCCCAGTGCGCGCCGTGCGCCCTGCGCGCCCGCCGCGCGC | | |
| | GCCGCTGCTACCTGCTGAGCGTGGCGCTGCGCCTGCTGGCCGAGCTGACCTTCCTGGG | | |
| | CGGCCAGGCGCTGCTCTACGGCTTCCGCGTGGCCCCGCACTTCGCGTGCGCCGGTCCG | | |
| | CCCTGCCCGCACACGGTCGACTGCTTCGTGAGCCGCCCCACCGAGAAGACCGTCTTCG | | |
| | TGCTCTTCTATTTCGCGGTGGGGCTGCTGTCGGCGCTGCTCAGCGTAGCCGAGCTGGG | | |
| | CCACCTGCTCTGGAAGGGCCGCCCGCGCGCCGGGGAGCGTGACAACCGCTGCAACCGT | | |
| | GCACACGAAGAGGCGCAGAAGCTGCTCCCGCCGCCGCCGCCGCCACCTCCCCCACCGG | | |
| | CCCTGCCCTCCCGGCGCCCCGGCCCCGAGCCGTGCGCCCCGCCGGCCTATGCGCACCC | | |
| | GGCGCCGGCCAGCCTCCGCGAGTGCGGCAGCGGCCGCGGCAGGAATGCGCCAATGGCT | | |
| | CCCAGATGTGGACCCCACCGCTTAACCCCTTACCCCCCAGCCGCGCTCCCCCAAGGGC | | |
| | CTTCCAGCCTGAGCCCCGCCAACAGCAGGGAGCTCTGCCCAGGTGAGAACCAGCCCAG | | |
| | GACTGGAGTCAGCGCCAGCCCGCCCCTAGTGCCCACGGACACCTCCCAACCTAGATCC | | |
| | TACCTGTCTTCCTTCCTTGAGGCTGGAGGGGAAGGCTCATGGACACAAGAATGCAAGC | | |
| | ATGCATGCACACAGCTACACTGCCTCCCATCCCCTCCCGCCGACGCTGCCAGGGTGCC | | |
| | CCTCCCTCGCTCCCCATCCTGGCAGGGCGGGCGGCGCAGAGCGCTCCACTCCGGATTC | | |
| | CCCACGCCCCCGAGCCGTTCGCAGGCTCGCACAAG | | |
| | ORF Start: ATG at 10 | | ORF Stop: AG at 1252 |
| | SEQ ID NO:112 | 414 aa | MW at 44773.0 kD |

| | |
|---|---|
| NOV34a, | MGEWAFLGSLLDAVQLQSPLVGRLWLVVMLIFRILVLATVGGAVFEDEQEEFVCNTLQ |
| CG59315-01 Protein Sequence | |
| | PGCRQTCYDRAFPVSHYRFWLFHILLLSAPPVLFVVYSMHRAGKEAGGAEAAAQCAPG |
| | LPEAQCAPCALRARRARRCYLLSVALRLLAELTFLGGQALLYGFRVAPHFACAGPPCP |
| | HTVDCFVSRPTEKTVFVLFYFAVGLLSALLSVAELGHLLWKGRPRAGERDNRCNRAHE |
| | EAQKLLPPPPPPPPPPALPSRRPGPEPCAPPAYAHPAPASLRECGSGRGRNAPMAPRC |
| | GRHRLTPYPPAALPQGPSSLSPANSRELCPGENQPRTGVSASPPLVPTDTSQPRSYLS |
| | SFLEAGGEGSWTQECKHACTQLHCLPSPPADAARVPLPRSPSWQGGRRRALHSGFPTP |
| | PSRSQART |

Further analysis of the NOV34a protein yielded the following properties shown in Table 34B.

TABLE 34B
Protein Sequence Properties NOV34a

| | |
|---|---|
| PSort | 0.6000 probability located in plasma membrane; 0.4000 |
| analysis: | probability located in Golgi body; 0.3000 probability |
| | located in endoplasmic reticulum (membrane); |
| | 0.0300 probability located in mitochondrial inner membrane |
| SignalP | Likely cleavage site between residues 39 and 40 |
| analysis: | |

A search of the NOV34a protein against the Geneseq database, a proprietary database that contains sequences published in patents and patent publication, yielded several homologous proteins shown in Table 34C.

TABLE 34C
Geneseq Results for NOV34a

| | | NOV34a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAW49009 | Mouse alpha 3 connexin protein- | 1 . . . 296 | 121/334 (36%) | 5e−52 |
| | Mus sp, 417 aa. [WO9830677-A1, | 1 . . . 327 | 169/334 (50%) | |
| | 16 JUL. 1998] | | | |
| AAW23968 | Connexin protein Cx40-Homo | 1 . . . 215 | 93/233 (39%) | 9e−46 |
| | sapiens, 358 aa. [WO9802150-A1, | 1 . . . 232 | 133/233 (56%) | |
| | 22 JAN. 1998] | | | |
| AAW23970 | Connexin protein Cx45-Homo | 4 . . . 212 | 93/252 (36%) | 3e−43 |
| | sapiens, 396 aa. [WO9802150-A1, | 3 . . . 253 | 137/252 (53%) | |
| | 22 JAN. 1998] | | | |
| AAW23969 | Connexin protein Cx43-Homo | 1 . . . 216 | 86/235 (36%) | 1e−42 |
| | sapiens, 382 aa. [WO9802150-A1, | 1 . . . 235 | 130/235 (54%) | |
| | 22 JAN. 1998] | | | |
| AAM93194 | Human polypeptide, SEQ ID NO: | 7 . . . 384 | 129/409 (31%) | 8e−38 |
| | [EP1130094-A2, 5 SEP. 2001] | 7 . . . 360 | 169/409 (40%) | |

In a BLAST search of public sequence databases, the NOV34a protein was found to have homology to the proteins shown in the BLASTP data in Table 34D.

TABLE 34D
Public BLASTP Results for NOV34a

| | | NOV34a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q91YD1 | CONNEXIN30.2-Mus musculus | 1 . . . 283 | 228/283 (80%) | e−129 |
| | (Mouse), 278 aa. | 1 . . . 265 | 240/283 (84%) | |
| I46053 | connexin44-bovine, 402 aa. | 1 . . . 397 | 151/418 (36%) | 1e−62 |
| | | 1 . . . 396 | 207/418 (49%) | |
| P41987 | Gap junction alpha-3 protein | 2 . . . 397 | 150/417 (35%) | 4e−62 |
| | (Connexin 44) (Cx44)-Bos | 1 . . . 395 | 206/417 (48%) | |
| | taurus (Bovine), 401 aa. | | | |
| AAA50954 | CONNEXIN44-Bos taurus | 1 . . . 398 | 154/429 (35%) | 1e−60 |
| | (Bovine), 407 aa. | 1 . . . 402 | 214/429 (48%) | |
| Q9TU17 | GAP JUNCTION PROTEIN | 1 . . . 398 | 147/415 (35%) | 1e−60 |
| | (CONNEXIN)-Ovis aries | 1 . . . 408 | 204/415 (48%) | |
| | (Sheep), 413 aa. | | | |

PFam analysis predicts that the NOV34a protein contains the domains shown in the

TABLE 34E
Domain Analysis of NOV34a

| | | Identities/ | |
|---|---|---|---|
| Pfam | NOV34a Match | Similarities for | Expect |
| Domain | Region | the Matched Region | Value |
| DUF26: domain 1 of 1 | 107 . . . 152 | 12/56 (21%) | 1.4 |
| | | 27/56 (48%) | |
| connexin: domain 1 of | 1 . . . 212 | 101/247 (41%) | 6.5e−75 |
| 1 | | 150/247 (61%) | |

### Example 35

The NOV35 clone was analyzed, and the nucleotide and predicted polypeptide sequences are shown in Table 35A.

TABLE 35A
NOV35 Sequence Analysis

| | SEQ ID NO:113 | 724 bp | |
|---|---|---|---|
| NOV35a, | TAAATTCGCGGCCGCGTCGACCTTCCGCAGACTCAACTGAGAAGTCAGCCTCTGCGGC | | |
| CG59203-01 DNA Sequence | | | |
| | AGGCACCAGGAATCTGCCTTTTCAGTTCTGTCTCCGGCAGGCTTTGAGGATGAAGGCT | | |
| | GCGGGCATTCTGACCCTCATTGGCTGCCTGGTCACAGGCGCCGAGTCCAAAATCTACA | | |
| | CTCGTTGCAAACTGGCAAAAATATTCTCGAGGGCTGGCCTGGACAATTACTGGGGCTT | | |
| | CAGCCTTGGAAACTGGATCTGCATGGCGTATTATGAGAGCGGCTACAACACCACAGCC | | |
| | CAGACGGTCCTGGATGACGGCAGCATCGACTACGGCATCTTCCAGATCAACAGCTTCG | | |
| | CGTGGTGCAGACGCGGAAAGCTGAAGGAGAACAACCACTGCCACGTCGCCTGCTCAGC | | |
| | CTTGGTCACTGATGACCTCACAGATGCGATTATCTGTGCCAAGAAAATTGTTAAAGAG | | |
| | ACACAAGGAATGAACTATTGGCAAGGCTGGAAGAAACACTGTGAGGGGAGAGACCTGT | | |
| | CCGACTGGAAAAAAGACTGTGAGGTTTCCTAAACTGGAACTGGACCCAGGATGCTTTG | | |
| | CAGCAACGCCCTAGGGTTTGCAGTGAATGTCCAAATGCCTGTGTCATCTTGTCCCGTT | | |
| | TCCTCCCAATATTCCTTCTCAAACTTGGAGAGGGAAAATTAAGCTATACTTTTAAGAA | | |
| | AATAAATATTTCCATTTAAATGTCAAAA | | |
| | ORF Start: ATG at 108 | | ORF Stop: TAA at 552 |
| | SEQ ID NO:114 | 148 aa | MW at 16655.9 kD |
| NOV35a, | MKAAGILTLIGCLVTGAESKIYTRCKLAKIFSRAGLDNYWGFSLGNWICMAYYESGYN | | |
| CG59203-01 Protein Sequence | | | |
| | TTAQTVLDDGSIDYGIFQINSFAWCRRGKLKENNHCHVACSALVTDDLTDAIICAKKI | | |
| | VKETQGMNYWQGWKKHCEGRDLSDWKKDCEVS | | |
| | SEQ ID NO:115 | 453 bp | |
| NOV35b, | CATTCTGACCCTCATTGGCTGCCTGGTCACAGGCGCCGAGTCCAAAATCTACACTCGT | | |
| CG59203-02 DNA Sequence | | | |
| | TGCAAACTGGCAAAAATATTCTCGAGGGCTGGCCTGGACAATTACTGGGGCTTCAGCC | | |
| | TTGGAAACTGGATCTGCATGGCGTATTATGAGAGCGGCTACAACACCACAGCCCAGAC | | |
| | GGTCCTGGATGACGGCAGCATCGACTACGGCATCTTCCAGATCAACAGCTTCGCGTGG | | |
| | TGCAGACGCGGAAAGCTGAAGGAGAACAACCACTGCCACGTCGCCTGCTCAGCCTTGG | | |
| | TCACTGATGACCTCACAGATGCAATTATCTGTGCCAGGAAAATTGTTAAAGAGACACA | | |
| | AGGAATGAATTATTGGCAAGGCTGGAAGAAACATTGTGAGGGCAGAGACCTGTCCGAC | | |
| | TGGAAAAAAGGCTGTGAGGTTTCCTAAACTGGAACTGGACCCAGGAT | | |
| | ORF Start: ATG at 134 | | ORF Stop: TAA at 431 |
| | SEQ ID NO:116 | 99 aa | MW at 11288.6 kD |

| | |
|---|---|
| NOV35b, | MAYYESGYNTTAQTVLDDGSIDYGIFQINSFAWCRRGKLKENNHCHVACSALVTDDLT |
| CG59203-02 Protein Sequence | |
| | DAIICARKIVKETQGMNYWQGWKKHCEGRDLSDWKKGCEVS |

Sequence comparison of the above protein sequences yields the following sequence relationships shown in Table 35B.

TABLE 35B
Comparison of NOV35a against NOV35b.

| | | Identities/ |
|---|---|---|
| Protein | NOV35a Residues/ | Similarities for |
| Sequence | Match Residues | the Matched Region |
| NOV35b | 50 . . . 148 | 97/99 (97%) |
| | 1 . . . 99 | 98/99 (98%) |

Further analysis of the NOV35a protein yielded the following properties shown in Table 35C.

TABLE 35C
Protein Sequence Properties NOV35a

| | |
|---|---|
| Psort | 0.3700 probability located in outside; 0.1697 probability |
| analysis: | located in microbody (peroxisome); 0.1000 probability located |
| | in endoplasmic reticulum (membrane); 0.1000 probability |
| | located in endoplasmic reticulum (lumen) |
| SignalP | Likely cleavage site between residues 20 and 21 |
| analysis: | |

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAY57399 | Human lysoenzyme LYC2 | 1 . . . 148 | 143/148 (96%) | 3e−86 |
| | polypeptide-Homo sapiens, 148 aa. | 1 . . . 148 | 147/148 (98%) | |
| | [WO200012722-A1, | | | |
| | 9 MAR. 2000] | | | |
| AAU29169 | Human PRO polypeptide sequence | 1 . . . 148 | 143/148 (96%) | 6e−86 |
| | #146-Homo sapiens, 148 aa. | 1 . . . 148 | 146/148 (98%) | |
| | [WO200168848-A2, | | | |
| | 20 SEP. 2001] | | | |
| AAB66145 | Protein of the invention #57- | 1 . . . 148 | 143/148 (96%) | 6e−86 |
| | Unidentified, 148 aa. | 1 . . . 148 | 146/148 (98%) | |
| | [WO200078961-A1, | | | |
| | 28 DEC. 2000] | | | |
| AAY99396 | Human PRO1278 (UNQ648) | 1 . . . 148 | 143/148 (96%) | 6e−86 |
| | amino acid sequence SEQ ID NO: | 1 . . . 148 | 146/148 (98%) | |
| | 203-Homo sapiens, 148 aa. | | | |
| | [WO200012708-A2, | | | |
| | 9 MAR. 2000] | | | |
| AAY71109 | Human Hydrolase protein-7 | 1 . . . 148 | 142/148 (95%) | 1e−85 |
| | (HYDRL-7)-Homo sapiens, 194 | 47 . . . 194 | 146/148 (97%) | |
| | aa. [WO200028045-A2, | | | |
| | 18 MAY 2000] | | | |

In a BLAST search of public sequence databases, the NOV35a protein was found to have homology to the proteins shown in the BLASTP data in Table 35E.

TABLE 35E
Public BLASTP Results for NOV35a

| | | NOV35a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96LF2 | BA14C22.1 (NOVEL PROTEIN | 1 . . . 148 | 148/148 (100%) | 7e−88 |
| | SIMILAR TO LYSOZYME)- | 1 . . . 148 | 148/148 (100%) | |
| | Homo sapiens (Human), 148 aa. | | | |
| Q9H1R9 | BA534G20.1.1 (NOVEL PROTEIN | 1 . . . 148 | 144/148 (97%) | 4e−86 |
| | SIMILAR TO LYSOZYME C-1 | 1 . . . 148 | 147/148 (99%) | |
| | (1,4-BETA-N- | | | |
| | ACYLMURAMIDASE C, EC | | | |
| | 3.2.1.17) (ISOFORM 1))-Homo | | | |
| | sapiens (Human), 148 aa. | | | |
| AAH21730 | HYPOTHETICAL 21.6 KDA | 1 . . . 148 | 143/148 (96%) | 2e−85 |
| | PROTEIN-Homo sapiens | 47 . . . 194 | 146/148 (98%) | |
| | (Human), 194 aa. | | | |
| Q9CPX3 | 1700038F02RIK PROTEIN-Mus | 1 . . . 148 | 110/148 (74%) | 3e−66 |
| | musculus (Mouse), 148 aa. | 1 . . . 148 | 127/148 (85%) | |
| Q9H1R8 | BA534G20.1.2 (NOVEL PROTEIN | 20 . . . 125 | 104/106 (98%) | 1e−59 |
| | SIMILAR TO LYSOZYME C-1 | 1 . . . 106 | 106/106 (99%) | |
| | (1,4-BETA-N- | | | |
| | ACYLMURAMIDASE C, EC | | | |
| | 3.2.1.17) (ISOFORM 2))-Homo | | | |
| | sapiens (Human), 106 aa (fragment). | | | |

PFam analysis predicts that the NOV35a protein contains the domains shown in the Table 35F.

TABLE 35F
Domain Analysis of NOV35a

| | | Identities/ | |
|---|---|---|---|
| Pfam | NOV35a Match | Similarities for | Expect |
| Domain | Region | the Matched Region | Value |
| lys: domain 1 of 1 | 20 . . . 145 | 68/129 (53%) | 8e−58 |
| | | 107/129 (83%) | |

### Example 36

The NOV36 clone was analyzed, and the nucleotide and predicted polypeptide sequences are shown in Table 36A.

TABLE 36A
NOV36 Sequence Analysis

| | SEQ ID NO:117 | 712 bp | |
|---|---|---|---|
| NOV36a, | GCAGCTATTGCACTTAATCGCGGCTGCTAGCACCATGTCCCGCGTTTTGGTGCCTTGC | | |
| CG58662-01 DNA Sequence | | | |
| | CATGTGAAAGGCACCGTAGCCCTGCAGGTGGGGGACGTATGGACCTCCCAAGGCCGGC | | |
| | CTAGTGTGCTGGTCATTGATGTCACCTTCCCCTGTGTCACTCCGTTCGAGGGGATCAC | | |
| | ATTTAAGAATTATTACACAGCGTTTTTTGAGCATCCTGTCTGTCAGCACACCTCAGCA | | |
| | CACACACCGGCCAAGTGGGTGACCTGCCTGTGGGACTACTGTCTGATGCCCGACCCAC | | |
| | ACAGTGAGGAGGGAGCCCAGGAGTATGTGTCGCTGTTCAAGCAACAGATACTGTGTGA | | |
| | CATGGCCAGAATATCGGACCTACACCTGATTCTGCAGCAGCCATCACCACTGTGGCTG | | |
| | TCTTTCACAGTGGACGAGCTGCAGATCTATCAGCAGGGACCAAAGAGCCCCTCCATGA | | |
| | TCTTCCCCAAGTGGCTCTCCCACCCAGTGCCCTGTGAGCAACCTGCACTCCTCCATGA | | |
| | GGGTCTCCCAGACCCCAGCAGGGTATCCTCTGAGGTGCAGCAGATGTGGGCACTGACA | | |
| | GAGATGATCCGGGCCAGTCACACCTCCGCGAGGATAGGCCACTTTGATGTAGATGGCT | | |
| | GTTATGACCTGAACTTACTCTCCTACACTTGAGTGGTGGCTCCTAGCCAAGATGTTGG | | |
| | CCTTTCTGTGCCCACT | | |
| | ORF Start: ATG at 35 | | ORF Stop: TGA at 668 |
| | SEQ ID NO:118 | 211 aa | MW at 23932.3 kD |
| NOV36a, | MSRVLVPCHVKGTVALQVGDVWTSQCRPSVLVIDVTFPCVTPFEGITFKNYYTAFFEH | | |
| CG58662-01 Protein Sequence | | | |
| | PVCQETSAHTPAKWVTCLWDYCLMPDPHSEEGAQEYVSLPKQQILCDMARISELHLIL | | |
| | QQPSPLWLSFTVEELQIYQQGPKSPSMIFPKWLSHPVPCEQPALLHEGLPDPSRVSSE | | |
| | VQQMWALTEMIRASRTSARIGEFDVDGCYDLNLLSYT | | |
| | SEQ ID NO:119 | 843 bp | |
| NOV36b, | CTGGCCTGAAGCCATGTCCCGCGTTCTAGCACCATGTCCCGCGTCTAGCACCATGTCC | | |
| CG58662-02 DNA Sequence | | | |
| | CGCGTCTAGCACCATGTCCCGCGTTCTAGCACCATGTCCCGCGTTCTAGCACCATGTC | | |
| | CCGCGTTCTAGCACCATGTCCCGCGTTTTGGTGCCTTGCCATGTGAAAGGCTCCGTAG | | |
| | CCCTCCAGGTGGGCGACGTGCGGACCTCCCAAGGCCGGCCTGGCGTGCTGGTCATCGA | | |
| | TGTCACCTTCCCCAGCGTCGCTCCCTTCGAGTTGCAGGAAATCACGTTTAAGAATTAC | | |
| | TACACAGCTTTTTTGAGCATCCGTGTCCGTCAGTACACCTCAGCACACACACCTGCCA | | |
| | AGTGGGTGACCTGCCTTCGGGACTACTGCCTGATGCCTGACCCACACAGTGAAGAGGG | | |
| | AGCCCAGGAGTATGTATCGCTGTTCAAGCATCAGATGCTATGTGACATGGCTAGAATA | | |
| | TCGGAGCTACGCCTGATTCTGCGGCAGCCATCACCACTGTGGCTGTCTTTCACAGTGG | | |
| | AGGAGCTGCAGATCTATCAGCAGGGACCAAAGAGCCCCTCCGTGACCTTTCCCAAGTG | | |
| | GCTCTCCCACCCAGTGCCCTGTGAGCAACCTGCACTCCTCCGTGAGGGTTTCCCAGAC | | |
| | CCCAGCAGGGTATCCTCCGAGGTGCAGCAGATGTGGGCACTGACAGAGATGATCCGGG | | |
| | CCAGTCACACCTCCGCAAGGATCGGCCGCTTTGATGTGGATGGCTGTTATGACCTGAC | | |
| | CTTGCTCTCCTACACTTGAATGGTTGCTCTTAGCCAAGATGTTGGCCTTTTTGTGGGC | | |
| | ACAGAAAGGCCAACGCGGGACATGGTGCTAG | | |
| | ORF Start: ATG at 132 | | Stop: TGA at 771 |
| | SEQ ID NO:120 | 213 aa | MW at 24222.6 kD |

| | |
|---|---|
| NOV36b, | MSRVLVPCHVKGSVALQVGDVRTSQGRPGVLVIDVTFPSVAPFELQEITFKNYYTAFL |
| CG58662-02 Protein Sequence | |
| | SIRVRQYTSAHTPAKWVTCLRDYCLMPDPHSEEGAQEYVLSFKHQMLCDMARISELRL |
| | ILRQPSPLWLSFTVEELQIYQQGPKSPSVTFPKWLSHPVPCEQPALLREGFPDPSRVS |
| | SEVQQMWALTEMIRASHTSARIGRFDVDGCYDLTLLSYT |

Sequence comparison of the above protein sequences yields the following sequence relationships shown in Table 36B.

TABLE 36B
Comparison of NOV36a against NOV36b.

| | | Identities/ |
|---|---|---|
| Protein | NOV36a Residues/ | Similarities for |
| Sequence | Match Residues | the Matched Region |
| NOV36b | 1 . . . 211 | 188/213 (88%) |
| | 1 . . . 213 | 193/213 (90%) |

Further analysis of the NOV36a protein yielded the following properties shown in Table 36C.

TABLE 36C
Protein Sequence Properties NOV36a

| | |
|---|---|
| PSort | 0.5666 probability located in microbody (peroxisome); |
| analysis: | 0.4500 probability located in cytoplasm; 0.1562 |
| | probability located in lysosome (lumen); 0.1000 |
| | probability located in mitochondrial matrix space |
| SignalP | No Known Signal Sequence Predicted |
| analysis: | |

A search of the NOV36a protein against the Geneseq database, a proprietary database that contains sequences published in patents and patent publication, yielded several homologous proteins shown in Table 36D.

TABLE 36D
Geneseq Results for NOV36a

| | | NOV36a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG04038 | Human secreted protein, SEQ ID | 1 . . . 103 | 82/105 (78%) | 1e−39 |
| | NO: 8119-Homo sapiens, 115 aa. | 1 . . . 105 | 85/105 (80%) | |
| | [EP1033401-A2, 6 SEP. 2000] | | | |

In a BLAST search of public sequence databases, the NOV36a protein was found to have homology to the proteins shown in the BLASTP data in Table 36E.

TABLE 36E
Public BLASTP Results for NOV36a

| | | NOV36a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9BSH3 | SIMILAR TO RIKEN CDNA | 1 . . . 211 | 190/213 (89%) | e−107 |
| | 1500032A17 GENE-Homo | 1 . . . 213 | 195/213 (91%) | |
| | sapiens (Human), 213 aa. | | | |
| Q9CQM0 | 1500032A17RIK PROTEIN- | 1 . . . 211 | 174/213 (81%) | 4e−97 |
| | Mus musculus (Mouse), | 1 . . . 213 | 183/213 (85%) | |
| | 213 aa. | | | |

PFam analysis predicts that the NOV36a protein contains the domains shown in the Table 36F.

TABLE 36F
Domain Analysis of NOV36a

| | | | |
|---|---|---|---|
| | | Identities/ | |
| Pfam | NOV36a | Similarities for | Expect |
| Domain | Match Region | the Matched Region | Value |

No Significant Matches Found

### Example 37

The NOV37 clone was analyzed, and the nucleotide and predicted polypeptide sequences are shown in Table 37A.

TABLE 37A
NOV37 Sequence Analysis

| | SEQ ID NO:121 | 520 bp | |
|---|---|---|---|
| NOV37a, | CATTTGCTGTCTCCTCTGCTCACCAGCAGCTGTACTGGAGCCACCCGCGAAAATTCGG | | |
| CG58584-01 DNA Sequence | | | |
| | CCAGGGTTCTCGCTCTTGTCGTGTCTGTTCAAACCGGCACGGTCTGATCCGGAAATAT | | |
| | GGCCTCAATATGTGCCGCCAGTGTTTCCGTCAGTACGCGAAGGATATCGGTTTCATTA | | |
| | AGAAAGACCTGAGCTGTCTTCCTTGGCACTGCCTATGGAGGTGACACCCATCTCCTCC | | |
| | ATCATGGCCATCCTGAGACCGCTCGCGAAGCCCAAGATCATCAAAAAGAGCACCAAGT | | |
| | TCACTGGGAACCAGTCAGACTGATATGTCAAAATTAAGGGTAACTGGTGGAAACACAG | | |
| | AGGTATTGACAACAGGGTTCATAGAAGGTTTGAGGGCCAGATCTATGCCCAACATTGG | | |
| | TTATGGGAGAAACAAAAAGACAAAGCACATACTGCCCAGTGGCTTCTGGAAGTTCCTC | | |
| | GTCCACAACGTTAAGGAGCTGGAAGTACTGCTGGTGAGCAGAGGAGACAGCAAATG | | |
| | ORF Start: TTT at 3 | | ORF Stop: TGA at 216 |
| | SEQ ID NO:122 | 71 aa | MW at 8461.8 kD |

| | |
|---|---|
| NOV37a, | FAVSSAHQQLYWSHPRKFGQGSRSCRVCSNRHGLIRKYGLNMCRQCFRQYAKDIGFIK |
| CG58584-01 Protein Sequence | |
| | KDLSCLPWHCLWR |

Further analysis of the NOV37a protein yielded the following properties shown in Table 37B.

TABLE 37B
Protein Sequence Properties NOV37a

| | |
|---|---|
| PSort | 0.6400 probability located in microbody (peroxisome); |
| analysis: | 0.4500 probability located in cytoplasm; 0.1000 |
| | probability located in mitochondrial matrix |
| | space; 0.1000 probability located in lysosome (lumen) |
| SignalP | No Known Signal Sequence Predicted |
| analysis: | |

A search of the NOV37a protein against the Geneseq database, a proprietary database that contains sequences published in patents and patent publication, yielded several homologous proteins shown in Table 37C.

TABLE 37C
Geneseq Results for NOV37a

| | | NOV37a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG76128 | Human colon cancer antigen | 7 . . . 60 | 46/54 (85%) | 4e−24 |
| | protein SEQ ID NO: 6892-Homo | 2 . . . 55 | 48/54 (88%) | |
| | sapiens, 80 aa. [WO200122920- | | | |
| | A2, 5 APR. 2001] | | | |
| AAM79084 | Human protein SEQ ID NO 1746- | 7 . . . 60 | 39/54 (72%) | 2e−18 |
| | Homo sapiens, 56 aa. | 3 . . . 56 | 43/54 (79%) | |
| | [WO200157190-A2, | | | |
| | 9 AUG. 2001] | | | |
| AAG39921 | Arabidopsis thaliana protein | 7 . . . 63 | 40/57 (70%) | 2e−18 |
| | fragment SEQ ID NO: 49464- | 3 . . . 58 | 45/57 (78%) | |
| | Arabidopsis thaliana, 637 aa. | | | |
| | [EP1033405-A2, 6 SEP. 2000] | | | |
| AAM80068 | Human protein SEQ ID NO 3714- | 7 . . . 58 | 38/52 (73%) | 5e−18 |
| | Homo sapiens, 74 aa. | 22 . . . 73 | 42/52 (80%) | |
| | [WO200157190-A2, | | | |
| | 9 AUG. 2001] | | | |
| AAG34802 | Arabidopsis thaliana protein | 7 . . . 58 | 37/52 (71%) | 1e−17 |
| | fragment SEQ ID NO: 42406- | 3 . . . 54 | 42/52 (80%) | |
| | Arabidopsis thaliana, 56 aa. | | | |
| | [EP1033405-A2, 6 SEP. 2000] | | | |

In a BLAST search of public sequence databases, the NOV37a protein was found to have homology to the proteins shown in the BLASTP data in Table 37D.

TABLE 37D
Public BLASTP Results for NOV37a

| | | NOV37a | | |
|---|---|---|---|---|
| Protein | | Residues/ | Identities/ | |
| Accession | | Match | Similarities for | Expect |
| Number | Protein/Organism/Length | Residues | the Matched Portion | Value |
| BAB79485 | RIBOSOMAL PROTEIN S29- | 7 . . . 60 | 53/54 (98%) | 1e−27 |
| | Homo sapiens (Human), 56 aa. | 3 . . . 56 | 53/54 (98%) | |
| P30054 | 40S ribosomal protein S29-Homo | 7 . . . 60 | 53/54 (98%) | 1e−27 |
| | sapiens (Human),, 55 aa. | 2 . . . 55 | 53/54 (98%) | |
| Q9OYP2 | 40S RIBOSOMAL PROTEIN S29- | 7 . . . 60 | 52/54 (96%) | 2e−27 |
| | Ictalurus punctatus (Channel | 3 . . . 56 | 53/54 (97%) | |
| | catfish), 56 aa. | | | |
| AAL62474 | RIBOSOMAL PROTEIN S29- | 7 . . . 60 | 41/54 (75%) | 6e−21 |
| | Spodoptera frugiperda (Fall | 3 . . . 56 | 48/54 (87%) | |
| | armyworm), 56 aa. | | | |
| Q9VH69 | CG8495 PROTEIN-Drosophila | 10 . . . 60 | 41/51 (80%) | 3e−20 |
| | melanogaster (Fruit fly), 56 aa. | 6 . . . 56 | 46/51 (89%) | |

PFam analysis predicts that the NOV37a protein contains the domains shown in the Table 37E.

TABLE 37E
Domain Analysis of NOV37a

| | | Identities/ | |
|---|---|---|---|
| Pfam | NOV37a Match | Similarities for | Expect |
| Domain | Region | the Matched Region | Value |
| Ribosomal_S14: | 7 . . . 61 | 17/60 (28%) | 7.5e−20 |
| domain 1 of 1 | | 51/60 (85%) | |

### Example 38

The NOV38 clone was analyzed, and the nucleotide and predicted polypeptide sequences are shown in Table 38A.

TABLE 38A
NOV38 Sequence Analysis

| | SEQ ID NO:123 | 2039 bp | |
|---|---|---|---|
| NOV38a, | GCAGCACACCTGCTCTGTGACTGACACTCTTGCAGAAGTGGGGCCACTTCAGGGACAT | | |
| CG58538-01 DNA Sequence | | | |
| | GGACAAGGTGTTGTACCTGCTGTCACAGAGCCTGTTATCTGTTCAGAATGACCGAAGA | | |
| | AGCATGCCGAACACGGAGTCAGAAACGAGCGCTTGAACGGGACCCAACAGAGGACGAT | | |
| | GTGGAGAGCAAGAAAATAAAAATGGAGAGAGGATTGTTGGCTTCAGATTTAAACACTG | | |
| | ACGGAGACATGAGGGTGACACCTGAGCCCGCAGCAGGTCCAACCCAAGCATTGCTGAG | | |
| | GGCAACAGAGGCCACGGCCATGGCCATGGGCAGAGGCGAAGGGCTGGTGGGCGATGGG | | |
| | CCCGTGGACATGCGCACCTCACACAGTGACATGAAGTCCGAGAGGAGACCCCCCTCAC | | |
| | CTGACGTGATTGTGCTCTCCGACAACGAGCAGCCCTCGAGCCCGAGAGTGAATGGGCT | | |
| | GACCACCGTGGCCTTGAAGGAGACTAGCACCGAGGCCCTCATGAAAAGCAGTCCTGAA | | |
| | GAACGAGAAAGGATGATCAAGCAGCTGAAGGAAGAATTGAGGTTAGAAGAAGCAAAAC | | |
| | TCGTGTTGTTGAAAAAGTTGCGGCAGAGTCAAATACAAAAGGAAGCCACCGCCCAGAA | | |
| | GCCCACAGGTTCTGTTGGGAGCACCGTGACCACCCCTCCCCCGCTTGTTCGGGGCACT | | |
| | CAGAACATTCCTGCTGGCAAGCCATCACTCCAGACCTCTTCAGCTCGGATGCCCGGCA | | |
| | GTGTCATACCCCCGCCCCTGGTCCGAGGTGGGCAGCAGGCGTCCTCGAAGCTGGGGCC | | |
| | ACAGGCGAGCTCACAGGTCGTCATGCCCCCACTCGTCAGGGGGGCTCAGCAAATCCAC | | |
| | AGCATTAGGCAACATTCCAGCACAGGGCCACCGCCCCTCCTCCTGGCCCCCCGGGCGT | | |
| | CGGTGCCCAGTGTGCAGATTCAGGGACAGAGGATCATCCAGCAGGGCCTCATCCGCGT | | |
| | CGCCAATGTTCCCAACACCAGCCTGCTCGTCAACATCCCACAGCCCACCCCAGCATCA | | |
| | CTGAAGGGGACAACAGCCACCTCCGCTCAGGCCAACTCCACCCCCACTAGTGTGGCCT | | |
| | CTGTGGTCACCTCTGCCGAGTCTCCAGCAAGCCGACAGGCGGCCGCCAAGCTGGCGCT | | |
| | GCGCAAACAGCTGGAGAAGACGCTACTCGAGATCCCCCCACCCAAGCCCCCAGCCCCA | | |
| | GAGATGAACTTCCTGCCCAGCGCCGCCAACAACGAGTTCATCTACCTGGTCGGCCTGG | | |
| | AGGAGGTGGTGCAGAACCTACTGGAGACACAAGCAGGCAGGATGTCGGCCGCCACTGT | | |
| | GCTGTCCCGGGAGCCCTACATGTGTGCACAGTGCAAGACGGACTTCACGTGCCGCTGG | | |
| | CGGGAGGAGAAGAGCGGCGCCATCATGTGTGAGAACTGCATGACAACCAACCAGAAGA | | |
| | AGGCGCTCAAGGTGGAGCACACCAGCCGGCTGAAGGCCGCCTTTGTGAAGGCGCTGCA | | |
| | GCAGGAACAGGAGATTGAGCAGCGGCTCCTGCAGCAGGGCACGGCCCCTGCACAGGCC | | |
| | AAGGCCGAGCCCACCGCTGCCCCACACCCCGTGCTGAAGCAGGCCTCCAGCCAGCTGT | | |
| | CCCGGGGTTCGGCCACGACGCCCCGAGGTGTCCTGCACACGTTCAGTCCGTCACCCAA | | |
| | ACTGCAGAACTCAGCCTCGGCCACAGCCCTGGTCAGCAGGACCGGCAGACATTCTGAG | | |
| | AGAACCGTGAGCGCCGGCAAGGGCAGCGCCACCTCCAACTGGAAGAAGACGCCCCTCA | | |
| | GCACAGGCGGGACCCTTGCGTTTGTCAGCCCAAGCCTGGCGGTGCACAAGAGCTCCTC | | |
| | GGCCGTGGACCGCCAGCGAGAGTACCTCCTGGACATGATCCCACCCCGCTCCATCCCC | | |
| | CAGTCAGCCACGTGGAAATAGTGCGAGCCAGGCCCCGTGGAAGACGGGCTCCCTCCTC | | |
| | CCCCACCTGGCCCCTGGTCTAGAAGGACCCACTGCACCACCCTCCGCTGGCTCGGGAA | | |
| | GACACCGTG | | |
| | ORF Start: ATG at 106 | | ORF Stop: TAG at 1933 |
| | SEQ ID NO:124 | 609 aa | MW at 65295.8 kD |

| | |
|---|---|
| NOV38a, | MTEEACRTRSQKRALERDPTEDDVESKKIKMERGLLASDLNTDGDMRVTPEPGAGPTQ |
| CG58538-01 Protein Sequence | |
| | GLLRATEATAMAMGRGEGLVGDGPVDMRTSHSDMKSERRPPSPDVIVLSDNSQPSSPR |
| | VNGLTTVAIKETSTEALMKSSPEERERNIKQLKEELRLEEAKLVLLKKLRQSQIQKEA |
| | TAQKPTGSVGSTVTTPPPLVRGTQNIPAGKPSLQTSSARMPGSVIPPPLVRGGQQASS |
| | KLGPQASSQVVMPPLVRGAQQIHSIRQHSSTGPPPLLLAPRASVPSVQIQGQRIIQQG |
| | LIRVANVPNTSLLVNIPQPTPASLKGTTATSAQANSTPTSVASVVTSAESPASRQAAA |
| | KLALRKQLEKTLLEIPPPKPPAPEMNFLPSAANNEFIYLVGLEEVVQNLLETQAGRMS |
| | AATVLSREPYMCAQCKTDFTCRWREEKSGAIMCENCMTTNQKKALKVEHTSRLKAAEV |
| | KALQQEQEIEQRLLQQGTAFAQAKAEPTAAPHPVLKQASSQLSRGSATTPRGVLHTFS |
| | PSPKLQNSASATALVSRTGRHSERTVSAGKGSATSNWKKTPLSTGGTLAFVSPSLAVH |
| | KSSSAVDRQREYLLDMIPPRSIPQSATWK |

Further analysis of the NOV38a protein yielded the following properties shown in Table 38B.

TABLE 38B
Protein Sequence Properties NOV38a

| | |
|---|---|
| PSort | 0.4404 probability located in mitochondrial matrix space; |
| analysis: | 0.3000 probability located in microbody (peroxisome); 0.1257 |
| | probability located in mitochondrial inner membrane; 0.1257 |
| | probability located in mitochondrial intermembrane space |
| SignalP | No Known Signal Sequence Predicted |
| analysis: | |

A search of the NOV38a protein against the Geneseq database, a proprietary database that contains sequences published in patents and patent publication, yielded several homologous proteins shown in Table 38C.

TABLE 38C
Geneseq Results for NOV38a

| | | NOV38a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM00991 | Human bone marrow protein, SEQ | 1 . . . 471 | 217/504 (43%) | 2e−87 |
| | ID NO: 492-Homo sapiens, 502 | 4 . . . 473 | 290/504 (57%) | |
| | aa. [WO200153453-A2, | | | |
| | 26 JUL. 2001] | | | |
| AAM00944 | Human bone marrow protein, SEQ | 1 . . . 471 | 217/504 (43%) | 2e−87 |
| | ID NO: 420-Homo sapiens, 546 | 48 . . . 517 | 290/504 (57%) | |
| | aa. [WO200153453-A2, | | | |
| | 26 JUL. 2001] | | | |
| AAM00831 | Human bone marrow protein, SEQ | 1 . . . 197 | 84/217 (38%) | 1e−23 |
| | ID NO: 194-Homo sapiens, 266 | 47 . . . 262 | 110/217 (49%) | |
| | aa. [WO200153453-A2, | | | |
| | 26 JUL. 2001] | | | |
| AAM85818 | Human immune/haematopoietic | 417 . . . 471 | 41/55 (74%) | 7e−19 |
| | antigen SEQ ID NO: 13411-Homo | 1 . . . 55 | 49/55 (88%) | |
| | sapiens, 84 aa. [WO200157182- | | | |
| | A2, 9 AUG. 2001] | | | |

In a BLAST search of public sequence databases, the NOV38a protein was found to have homology to the proteins shown in the BLASTP data in Table 38D.

TABLE 38D
Public BLASTP Results for NOV38a

| | | | | |
|---|---|---|---|---|
| | | NOV38a | | |
| Protein | Protein/ | Residues/ | Identities/ | |
| Accession | Organism/ | Match | Similarities for | Expect |
| Number | Length | Residues | the Matched Portion | Value |

No Significant Matches Found

PFam analysis predicts that the NOV38a protein contains the domains shown in the Table 38E.

TABLE 38E
Domain Analysis of NOV38a

| | | Identities/ | |
|---|---|---|---|
| Pfam | NOV38a Match | Similarities for | Expect |
| Domain | Region | the Matched Region | Value |
| GATA: domain 1 of 1 | 414 . . . 453 | 12/43 (28%) | 1.1 |
| | | 17/43 (40%) | |

### Example 39

The NOV39 clone was analyzed, and the nucleotide and predicted polypeptide sequences are shown in Table 39A.

TABLE 39A
NOV39 Sequence Analysis

| | SEQ ID NO:125 | 1421 bp | |
|---|---|---|---|
| NOV39a, | ACCATTTCAGAGATGTCTTCCAGAAGTACCAAAGATTTAATTAAAAGTAAGTGGGGAT | | |
| CG59371-01 DNA Sequence | | | |
| | CGAAGCCTAGTAACTCCAAATCCGAAACTACATTAGAAAAATTAAAGGGAGAAATTGC | | |
| | ACACTTAAAGACATCAGTGGATGAAATCACAAGTGGGAAAGGAAAGCTGACTGATAAA | | |
| | GAGAGACACAGACTTTTGGAGAAAATTCGAGTCCTTGAGGCTGAGAAGGAGAAGAATG | | |
| | CTTATCAACTCACAGAGAAGGACAAAGAAATACAGCGACTGAGAGACCAACTGAAGGC | | |
| | CAGATATAGTACTACCACATTGCTTGAACAGCTGGAAGAGACAACGAGAGAAGGAGAA | | |
| | AGGAGGGAGCAGGTGTTGAAAGCCTTATCTGAAGAGAAAGACGTATTGAAACAACAGT | | |
| | TGTCTGCTGCAACCTCACGAATTGCTGAACTTGAAAGCAAAACCAATACACTCCGTTT | | |
| | ATCACAGACTGTGGCTCCAAACTGCTTCAACTCATCAATAAATAATATTCATGAAATG | | |
| | GAAATACAGCTGAAAGATGCTCTGGAGAAAAATCAGCAGTGGCTCGTGTATGATCAGC | | |
| | AGCGGGAAGTCTATGTAAAAGGACTTTTAGCAAAGATCTTTGAGTTGGAAAAGAAAAC | | |
| | GGAAACAGCTGCTCATTCACTCCCACAGCAGACAAAAAAGCCTGAATCAGAAGGTTAT | | |
| | CTTCAAGAAGAGAAGCAGAAATGTTACAACGATCTCTTGGCAACTGCAAAAAAAGATC | | |
| | TTGAGGTTGAACGACAAACCATAACTCAGCTGAGTTTTGAACTGAGTGAATTTCGAAG | | |
| | AAAATATGAAGAAACCCAAAAAGAAGTTCACAATTTAAATCAGCTGTTGTATTCACAA | | |
| | AGAAGGGCAGATGTGCAACATCTGGAAGATGATAGGCATAAAACAGAGAAGATACAAA | | |
| | AACTCAGGGAAGAGAATGATATTGCTAGGGGAAAACTTGAAGAAGAGAAGAAGAGATC | | |
| | CGAAGAGCTCTTATCTCAGGTCCAGTCTCTTTACACATCTCTGCTAAACCAGCAAGAA | | |
| | GAACAAACAAGGGTAGCTCTGTTGGAACAACAGATGCAGGCATGTACTTTAGACTTTG | | |
| | AAAATGAAAAACTCGACCGTCAACATGTGCAGCATCAATTGCATGTAATTCTTAAGGA | | |
| | GCTCCGAAAAGCAAGAAAAAATATAACACAGTTGGAATCCTTGAAACAGCTTCATGAG | | |
| | TTTGCCATCACAGAGCCATTAGTCACTTTCCAAGGAGAGACTGAAAACAGAGAAAAAG | | |
| | TTGCCGCCTCACCAAAAAGTCCCACTGCTGCACTCAATGGAAGCCTGGTGGAATGTCC | | |
| | CAAGTGCAATATACAGTATCCAGCCACTGAGCATCGCGATCTGCTTGTCCATGTGGAA | | |
| | TACTGTTCAAAGTAGCAAAATAAGTATTT | | |
| | ORF Start: ATG at 13 | | ORF Stop: TAG at 1405 |
| | SEQ ID NO:126 | 464 aa | MW at 54045.6 kD |

| | |
|---|---|
| NOV39a, | MSSRSTKDLIKSKWGSKPSNSKSETTLEKLKGEIAHLKTSVDEITSGKGKLTDKERHR |
| CG59371-01 Protein Sequence | |
| | LLEKIRVLEAEKEKNAYQLTEKDKEIQRLRDQLKARYSTTTLLEQLEETTREGERREQ |
| | VLKALSEEKDVLKQQLSAATSRIAELESKTNTLRLSQTVAPNCFNSSINNIHEMEIQL |
| | KDALEKNQQWLVYDQQREVYVKGLLAKTFELEKKTETAAHSLPQQTKKPSSEGYLQEE |
| | KQKCYNDLLASAKKDLEVERQTITQLSFELSEFRRKYEETQKEVHNLNQLLYSQRRAD |
| | VQHLEDDRHKTEKIQKLREENDIARGKLEEEKKRSEELLSQVQSLYTSLLKQQEEQTR |
| | VALLEQQAQACTLDFENEKLDRQHVQHQLNVILKELRKARKNITQLESLKQLHEFAIT |
| | EPLVTFQGETENREKVAASPKSPTAALNGSLVECPKCNIQYPATEHRDLLVHVEYCSK |

Further analysis of the NOV39a protein yielded the following properties shown in Table 39B.

TABLE 39B
Protein Sequence Properties NOV39a

| | |
|---|---|
| PSort | 0.4500 probability located in cytoplasm; 0.3000 |
| analysis: | probability located in microbody (peroxisome); |
| | 0.1000 probability located in mitochondrial matrix |
| | space; 0.1000 probability located in lysosome (lumen) |
| SignalP | No Known Signal Sequence Predicted |
| analysis: | |

A search of the NOV39a protein against the Geneseq database, a proprietary database that contains sequences published in patents and patent publication, yielded several homologous proteins shown in Table 39C.

TABLE 39C
Geneseq Results for NOV39a

| | | NOV39a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB92925 | Human protein sequence SEQ ID | 170 . . . 392 | 222/223 (99%) | e−122 |
| | NO: 11576-Homo sapiens, 231 aa. | 1 . . . 223 | 222/223 (99%) | |
| | [EP1074617-A2, 7 FEB. 2001] | | | |
| AAG75490 | Human colon cancer antigen | 1 . . . 67 | 64/67 (95%) | 1e−28 |
| | protein SEQ ID NO: 6254-Homo | 99 . . . 165 | 64/67 (95%) | |
| | sapiens, 165 aa. [WO200122920- | | | |
| | A2, 5 APR. 2001] | | | |
| AAM78520 | Human protein SEQ ID NO 1182- | 6 . . . 394 | 96/421 (22%) | 3e−12 |
| | Homo sapiens, 990 aa. | 515 . . . 929 | 182/421 (42%) | |
| | [WO200157190-A2, | | | |
| | 9 AUG. 2001] | | | |
| AAM41000 | Human polypeptide SEQ ID NO | 70 . . . 420 | 90/384 (23%) | 3e−12 |
| | 5931-Homo sapiens, 1988 aa. | 852 . . . 1203 | 161/384 (41%) | |
| | [WO200153312-A1, | | | |
| | 26 JUL. 2001] | | | |
| AAM40999 | Human polypeptide SEQ ID NO | 70 . . . 420 | 90/384 (23%) | 3e−12 |
| | 5930-Homo sapiens, 1988 aa. | 852 . . . 1203 | 161/384 (41%) | |
| | [WO200153312-A1, | | | |
| | 26 JUL. 2001] | | | |

In a BLAST search of public sequence databases, the NOV39a protein was found to have homology to the proteins shown in the BLASTP data in Table 39D.

TABLE 39D
Public BLASTP Results for NOV39a

| | | NOV39a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96H32 | SIMILAR TO RIKEN CDNA | 1 . . . 464 | 458/464 (98%) | 0.0 |
| | 1200008O12 GENE - Homo | 1 . . . 464 | 458/464 (98%) | |
| | sapiens (Human), 464 aa. | | | |
| Q9DBZ8 | 1200008O12RIK PROTEIN - | 1 . . . 464 | 348/464 (75%) | 0.0 |
| | Mus musculus (Mouse), 462 aa. | 1 . . . 462 | 401/464 (86%) | |
| Q9NVS7 | CDNA FLJ10540 FIS, CLONE | 170 . . . 392 | 222/223 (99%) | e−122 |
| | NT2RP2001245 - Homo sapiens | 1 . . . 223 | 222/223 (99%) | |
| | (Human), 231 aa. | | | |
| Q9CZP8 | 2700032M20RIK PROTEIN - | 1 . . . 176 | 121/176 (68%) | 3e−63 |
| | Mus musculus (Mouse), 189 aa. | 1 . . . 176 | 150/176 (84%) | |
| Q9VJE5 | CLIP-190 PROTEIN - Drosophila | 4 . . . 439 | 108/461 (23%) | 2e−16 |
| | melanogaster (Fruit fly), 1690 aa. | 675 . . . 1118 | 203/461 (43%) | |

PFam analysis predicts that the NOV39a protein contains the domains shown in the Table 39E.

TABLE 39E

| | | | |
|---|---|---|---|
| | | Identities/ | |
| | NOV39a | Similarities | Expect |
| Pfam Domain | Match Region | for the Matched Region | Value |

No Significant Matches Found

Table 39E. Domain Analysis of NOV39a Identities Pfam Domain NOV39a Match Region Similarities Expect Value for the Matched Region No Significant Matches Found

### Example 40

The NOV40 clone was analyzed, and the nucleotide and predicted polypeptide sequences are shown in Table 40A.

TABLE 40A
NOV40 Sequence Analysis

| | SEQ ID NO:127 | 3955 bp | |
|---|---|---|---|
| NOV40a, | TGCACCCTCGCTGCCTCCTTTCCTCCATGCTGCCTGGATCTGGCGAGCTGGGGTGATT | | |
| CG59346.01 DNA Sequence | | | |
| | AATTGGCTATGATGATGAACGTCCCCGGCGGAGGAGCGGCCGCGGTGATGATGACGGG | | |
| | CTACAATAATGGTCGCTGTCCCCGGAATTCTCTCTACAGTGACTGCATTATTGAGGAG | | |
| | AAGACGGTGGTCCTGCAGAAAAAAGACAATGAGGGCTTTGGATTCGTGCTTCGAGGGG | | |
| | CCAAAGCTGACACACCCATTGAAGAATTCACACCAACACCGGCTTTCCCAGCCCTACA | | |
| | GTACCTGGAGTCCGTGGATGAAGGTGGGGTGGCGTGGCAAGCCCGACTAAGGACCGGG | | |
| | GACTTCTTGATTGAGGTTAACAATGAGAATGTTGTCAAAGTCGGCCACAGGCAGGTGG | | |
| | TGAACATGATCCGGCAGGGAGGGAATCACCTGGTCCTTAAGGTGGTCACGGTGACCAG | | |
| | GAATCTGGACCCCGACGACACCGCCAGGAAGAAAGCTCCCCCGCCTCCAAAGCGGGCA | | |
| | CCGACCACAGCCCTCACCCTGCGCTCCAAGTCCATGACCTCGGAGCTGGAGGAGCTCG | | |
| | ATAAACCCGAGGAGATAGTCCCGGCCTCCAAGCCCTCCCGCGCTGCTGAGAACATGGC | | |
| | TGTGGAACCGAGGGTGGCGACCATCAAGCAGCGGCCCAGCAGCCGGTGCTTCCCGGCG | | |
| | GGCTCAGACATGAACGTGAGTGGCCGTACCTTGGGACCACGAGGGCGGGGGCCGACGG | | |
| | TGCCCCCTAGGCTCTCTGGTTTGCAGTCTGTGTACGAACGCCAAGGAATCGCCGTGAT | | |
| | GACGCCCACTGTTCCTGGGAGCCCAAAAGCCCCGTTTCTGGGCATCCCTCGAGGTACG | | |
| | ATGCGAAGGCAGAAATCAATAGGAATAACAGAGGAAGAGCGGCAGTTTCTGGCTCCTC | | |
| | CAATGCTGAAGTTCACCAGAAGCCTGTCCATGCCGGACACCTCTGAGGACATCCCCCC | | |
| | TCCACCGCAGTCTGTGCCCCCGTCCCCACCACCACCTTCCCCAACCACTTACAACTGC | | |
| | CCCAAGTCCCCAACTCCAAGAGTCTACGGGACGATTAAGCCTGCGTTCAATCAGAATT | | |
| | CTGCCGCCAAGGTGTCCCCCGCCACCAGGTCCGACACCGTGGCCACCATGATGAGGGA | | |
| | GAAGGGGATGTACTTCAGGAGAGAGCTGGACCGCTACTCCTTGGACTCTGAAGACCTC | | |
| | TACAGTCGGAATGCCGGCCCGCAAGCCAACTTCCGCAACAAGAGAGGCCAGATGCCAG | | |
| | AAAACCCATACTCAGACGTGGGGAAGATCGCCAGCAAAGCCGTCTACGTCCCCGCCAA | | |
| | GCCCGCCAGGCGGAAGGGGATGCTGGTGAAGCAGTCCAACGTGGAGGACAGCCCCGAG | | |
| | AAGACGTGCTCCATCCCTATCCCGACCATCATCGTGAAGGAGCCGTCCACCAGCAGCA | | |
| | GCGGCAAGAGCAGCCAGGGCAGCAGCATGGAGATCGACCCCCAGGCCCCGGAGCCACC | | |
| | GAGCCAGCTGCGGCCTGACGAAAGCCTCACCGTCAGCAGCCCCTTTGCCGCCGCCATC | | |
| | GCCGGAGCCGTCCGCGACCGTGAGAAGCGGCTGGAAGCCAGGAGGAACTCCCCGGCCT | | |
| | TCCTCTCCACAGACCTGGGGGATGAGGATGTGGGCCTGGGGCCACCCGCCCCCAGGAC | | |
| | GCGGCCCTCCATGTTCCCCGAGGAGGGGGATTTTGCTGACGAGGACAGCGCTGAGCAG | | |
| | CTGTCATCCCCCATGCCGAGTGCCACGCCCAGGGAGCCCGAAAACCATTTCGTGGGTG | | |
| | GCGCCGAGGCCAGTGCTCCGGGTGAGGCTGGGAGGCCGCTGAATTCCACGTCCAAAGC | | |
| | CCAGGGGCCCGAGAGCAGCCCAGCAGTGCCCTCCGCGAGCAGCGGCACAGCCGGCCCC | | |
| | GGGAATTATGTCCACCCACTCACAGGGCGGCTGCTTGATCCCAGCTCCCCGCTGGCCC | | |
| | TGGCACTCTCCGCAAGGGACCCAGCCATGAAGGAGTCTCAACAGGGACCCAAAGGGGA | | |
| | GGCCCCCAAGGCCGACCTCAACAAACCTCTTTACATTGATACCAAAATGCGGCCCAGC | | |
| | CTGGATGCCGGCTTCCCTACGGTCACCAGGCAGAACACCCGGGGACCCCTGAGGCGGC | | |
| | AGGAGACGGAGAACAAGTACGAGACCGACCTGGGCCGAGACCGGAAAGGCGATGACAA | | |
| | GAAGAACATGCTGATCGACATCATGGACACGTCCCAGCAGAAGTCGGCTGGCCTGCTG | | |
| | ATGGTGCACACCGTGGACGCCACTAAGCTGGACAACGCCCTGCAGGAAGAGGACGAGA | | |
| | AGGCAGAGGTGGAGATGAAGCCAGACAGCTCGCCGTCCGAGGTGCCAGAAGGTGTTTC | | |
| | CGAAACCGAAGGTGCTTTACAGATCTCCGCTGCCCCCGAGCCCACCACCGTGCCCGGC | | |
| | AGAACCATCGTCGCGGTGGGCTCCATGGAAGAGGCGGTGATTTTGCCATTCCGCATCC | | |
| | CTCCTCCCCCTCTGGCATCCGTGGACTTGGATGAGGATTTTATTTTTACAGAGCCATT | | |
| | GCCTCCTCCCCTGGAATTTGCAAATAGTTTTGATATCCCCGATGACCGGGCAGCTTCT | | |
| | GTCCCGGCTCTCTCAGACTTAGTGAAGCAGAACAAAAGCGACACCCCTCAGTCCCCTT | | |
| | CGTTGAACTCCAGCCAACCAACCAACTCTGCAGACAGCAAGAAGCCAGCCAGTCTTTC | | |
| | AAACTGTCTGCCTGCCTCATTCCTGCCACCCCCTGAAAGCTTTGACGCCGTCGCCGAC | | |
| | TCTGGGATCGAGGAGGTGGACAGCCGGAGTAGCAGCGACCACCACCTCGAGACGACCA | | |
| | GCACTATCTCCACCGTGTCTAGCATCTCCACCCTGTCTTCCGAAGGTGGAGAGAATGT | | |
| | GGACACCTGCACAGTCTATGCAGATGGGCAAGCATTTATGGTTGACAAACCCCCAGTA | | |
| | CCTCCTAAGCCAAAAATGAAGCCCATCATTCACAAAAGCAATGCACTTTATCAAGACG | | |
| | CGCTCGTGGAAGAAGATGTAGATAGCTTTGTTATCCCCCCGCCCGCTCCCCCGCCCCC | | |
| | GCCGGGCAGTGCCCAGCCTGGGATGGCCAAGGTTCTCCAGCCAAGGACCTCCAAGTTG | | |
| | TGGGGCGACGTCACAGAGATCAAAAGCCCGATTCTCTCAGGCCCAAAGGCAAACGTTA | | |
| | TTAGTGAATTGAACTCTATCCTACAGCAAATGAACCGAGAGAAATTGGCAAAGCCGGG | | |
| | GGAAGGACTGGATTCACCAATGGGAGCCAAGTCCGCCAGCCTCGCTCCAAGAAGCCCG | | |
| | GAGATCATGAGCACCATCTCAGGTACACGGAGCACGACGGTCACCTTCACTGTTCGCC | | |
| | CCGGCACCTCCCAGCCCATCACCCTGCAGAGCCGGCCCCCCGACTATGAAAGCAGGAC | | |
| | CTCAGGAACAAGACGTGCCCCAAGCCCTGTGGTCTCGCCAACACAGATGAACAAAGAG | | |
| | ACCCTGCCCGCCCCCCTGTCTGCTGCCACCGCCTCTCCTTCTCCCGCTCTCTCAGATG | | |
| | TCTTTAGCCTTCCAAGCCAGCCCCCTTCTGGGGATCTATTTGGCTTGAACCCAGCGGG | | |
| | ACGCAGTAGGTCGCCATCCCCCTCGATACTGCAACAGCCAATCTCAAATAAGCCTTTT | | |
| | ACAACTAAACCTGTCCACCTGTGGACTAAACCAGATGTGGCCGATTGGCTGGAAAGTC | | |
| | TAAACTTGGGTGAACATAAAGAGGCCTTCATGGACAATGAGATCGATGGCAGTCACTT | | |
| | ACCAAACCTGCAGAAGGAGGACCTCATCGATCTTGGGGTAACTCGAGTCGGGCACAGA | | |
| | ATGAACATAGAAAGGGCTTTGAAACAGCTGCTGGACAGATAAGGACGGCTGCTCTCCA | | |
| | CCTCGCAGACTGCTCTTGTTATAAGTAGAGATGGGCTCGTGCTGAAACATCTGAATGC | | |
| | CAAGCGAAGTC | | |
| | ORF Start: ATG at 67 | | ORF Stop: TAA at 3868 |
| | SEQ ID NO:128 | 1267 aa | MW at 136108.7 kD |

| | |
|---|---|
| NOV40a, | MMMNVPGGGAAAVMNTGYNNGRCPRNSLYSDCIIEEKTVVLQKKDNEGFGFVLRGAKA |
| CG59346-01 Protein | |
| Sequence | DTPIEEFTPTPAFPALQYLESVDEGGVAWQAGLRTGDFLIEVNNENVVKVGHRQVVNM |
| | IRQGGNHLVLKVVTVTRNLDPDDTARKKAPPPPKRAPTTALTLRSKSMTSELEELDKP |
| | EEIVPASKPSRAAENMAVEPRVATIKQRPSSRCFPAGSDNNVSGRTLGPRGRGPTVPP |
| | RLSGLQSVYERQGIAVMTPTVPGSPKAPFLGIPRGTMRRQKSTGTTEEERQFLAPPML |
| | KFTRSLSMPDTSEDIPPPPQSVPPSPFPPSPTTYNCPKSPTPRVYGTIKPAFNQNSAA |
| | KVSPATRSDTVATMMREKGMYFRRELDRYSLDSEDLYSRNAGPQANFRNKRGQMPENP |
| | YSEVGKIASKAVYVPAKPARRKGMLVKQSNVEDSPEKTCSIPTPTIIVKEPSTSSSGK |
| | SSQGSSMEIDPQAPEPPSQLRPDESLTVSSPFAAAIAGAVRDREKRLEARRNSPAFLS |
| | TDLGDEDVGLGPPAPRTRPSMFPEEGDFADEDSAEQLSSPMPSATPREPENHFVGGAE |
| | ASAPGEAGRPLNSTSKAQGPESSPAVPSASSGTAGPGNYVHPLTGRLLDPSSPLALAL |
| | SARDRAMKESQQGPKGEAPKADLNKPLYIDTKMRPSLDAGFPTVTRQNTRGPLRRQET |
| | ENKYETDLGRDRKGDDKKNNLIDIMDTSQQKSAGLLMVHTVDATKLDNALQEEDEKAE |
| | VEMKPDSSPSEVPEGVSETEGALQISAAPEPTTVPGRTIVAVGSMEEAVILPFRIPPP |
| | PLASVDLDEDFIFTEPLPPPLEFANSFDIPDDRAASVPALSDLVKQRKSDTPQSPSLN |
| | SSQPTNSADSKKPASLSNCLPASFLPPPESFDAVADSGIEEVDSRSSSDHHLETTSTI |
| | STVSSISTLSSEGGENVDTCTVYADGQAFMVDKPPVPPKPKNKPIIHKSNALYQDALV |
| | EEDVDSFVIPPPAPPPPPGSAQPGMAKVLQPRTSKLWGDVTEIKSPILSGPKANVISE |
| | LNSILQQMNREKLAKPGEGLDSPMGAKSASLAPRSPEIMSTISGTRSTTVTFTVRPGT |
| | SQPITLQSRPPDYESRTSGTRRAPSPVVSPTEMNKETLPAPLSAATASPSPALSDVFS |
| | LPSQPPSGDLFGLNPAGRSRSPSPSILQQPISNKPFTTKPVHLWTKPDVADWLESLNL |
| | GEHKEAFMDNEIDGSHLPNLQKEDLIDLGVTRVGHRMNIERALKQLLDR |

Further analysis of the NOV40a protein yielded the following properties shown in Table 40B.

TABLE 40B
Protein Sequence Properties NOV40a

| | |
|---|---|
| PSort | 0.4500 probability located in cytoplasm; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV40a protein against the Geneseq database, a proprietary database that contains sequences published in patents and patent publication, yielded several homologous proteins shown in Table 40C.

TABLE 40C
Geneseq Results for NOV40a

| | | NOV40a | Identities/ | | |
|---|---|---|---|---|---|
| | | Residues/ | Similarities for | | |
| Geneseq | Protein/Organism/Length | Match | the Matched | | Expect |
| Identifier | [Patent #, Data] | Residues | Region | | Value |
| AAM79240 | Human protein SEQ ID NO 1902 - | 14 . . . 1267 | 1231/1271 | (96%) | 0.0 |
| | Homo sapiens, 1248 aa. | 1 . . . 1248 | 1231/1271 | (96%) | |
| | [WO200157190-A2, 9 AUG | | | | |
| | 2001] | | | | |
| AAB31518 | Amino acid sequence of the rat | 30 . . . 1267 | 1078/1255 | (85%) | 0.0 |
| | Shank2 polypeptide - Rattus sp, | 240 . . . 1470 | 1132/1255 | (89%) | |
| | 1470 aa. [WO200078921-A2, | | | | |
| | 28 DEC 2000] | | | | |
| AAM80224 | Human protein SEQ ID NO 3870 - | 172 . . . 1267 | 1071/1103 | (97%) | 0.0 |
| | Homo sapiens, 1161 aa. | 82 . . . 1161 | 1071/1103 | (97%) | |
| | [WO200157190-A2, 9 AUG | | | | |
| | 2001] | | | | |
| AAB31517 | Amino acid sequence of the rat | 18 . . . 1264 | 496/1349 | (36%) | 0.0 |
| | Shank3a polypeptide - Rattus sp, | 550 . . . 1737 | 673/1349 | (49%) | |
| | 1740 aa. [WO200078921-A2, | | | | |
| | 28 DEC 2000] | | | | |
| AAY83017 | Rat shank 3a - Rattus rattus, 1740 | 18 . . . 1264 | 496/1349 | (36%) | 0.0 |
| | aa. [WO200011204-A2, | 550 . . . 1737 | 673/1349 | (49%) | |
| | 2 MAR 2000] | | | | |

In a BLAST search of public sequence databases, the NOV40a protein was found to have homology to the proteins shown in the BLASTP data in Table 40D.

TABLE 40D
Public BLASTP Results for NOV40a

| | | NOV40a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UPX8 | KIAA1022 PROTEIN - Homo | 124 . . . 1267 | 1121/1154 (97%) | 0.0 |
| | sapiens (Human), 1131 aa | 1 . . . 1131 | 1121/1154 (97%) | |
| | (fragment). | | | |
| Q9QX93 | PROLINE RICH SYNAPSE | 2 . . . 1267 | 1103/1276 (86%) | 0.0 |
| | ASSOCIATED PROTEIN 1 - | 1 . . . 1252 | 1158/1276 (90%) | |
| | Rattus norvegicus (Rat), 1252 aa. | | | |
| O70470 | CORTACTIN-BINDING | 2 . . . 1267 | 1102/1276 (86%) | 0.0 |
| | PROTEIN 1 - Rattus norvegicus | 1 . . . 1252 | 1158/1276 (90%) | |
| | (Rat), 1252 aa. | | | |
| Q9WUV9 | PROLINE RICH SYNAPSE | 2 . . . 1267 | 1103/1283 (85%) | 0.0 |
| | ASSOCIATED PROTEIN 1 - | 1 . . . 1259 | 1158/1283 (89%) | |
| | Rattus norvegicus (Rat), 1259 aa. | | | |
| Q9WUW0 | PROLINE RICH SYNAPSE | 2 . . . 1267 | 1095/1276 (85%) | 0.0 |
| | ASSOCIATED PROTEIN 1 - | 1 . . . 1250 | 1151/1276 (89%) | |
| | Rattus norvegicus (Rat), 1250 aa. | | | |

PFam analysis predicts that the NOV40a protein contains the domains shown in the Table 40E.

TABLE 40E
Domain Analysis of NOV40a

| | | Identities/ | |
|---|---|---|---|
| | | Similarities | |
| | NOV40a | for the | Expect |
| Pfam Domain | Match Region | Matched Region | Value |
| PDZ: domain 1 of 1 | 38 . . . 131 | 23/97 (24%) | 1e−07 |
| | | 70/97 (72%) | |
| SAM: domain 1 of 1 | 1202 . . . 1265 | 27/68 (40%) | 9.8e−22 |
| | | 53/68 (78%) | |

### Example 41

The NOV41 clone was analyzed, and the nucleotide and predicted polypeptide sequences are shown in Table 41A.

TABLE 41A
NOV41 Sequence Analysis

| | SEQ ID NO:129 | 2069 bp | |
|---|---|---|---|
| NOV41a, | GGACACTGACATGGACTGAAGGAGTAGAAAGCACTATAAATGTCTTTCCTTATCTGTG | | |
| CG57814-01 DNA Sequence | | | |
| | TGTACTCTTATCTCACTGTTCTATTTTTTCTCCTCATTTATATTAACTCTTTCTTACC | | |
| | TTTTTTTCTGAACTTCTAGGCCTTCTCTTTCCAGAACTGGTGGAAGACAAATGAAACG | | |
| | GCCAAGATGGTAAGAAACAAGCCGCATTTCTCCTTGGGGAGACTGATAATTTAAAAGG | | |
| | TTTGTTGTGTCAGAAACATTCCCAGCTTCATCACCAACCCTTTCCTTCCACCTCTGCC | | |
| | CACTGGAGACCACTTACATCCCGAAGCGGACGCGGCAGCTGAAGTCAGGAAACCATGC | | |
| | ATCACATTAGCAGGAGCCAACTGCAGACTTTAAACTCCGTTCAACATGTGGATGCGGC | | |
| | AGAGAAATGACCTGTCCAGACAAGCCGGGGCAGCTCATAAACTGGTTCATCTGCTCCC | | |
| | TGTGCGTCCCGCGGGTGCGTAAGCTCTGGAGCAGCCGGCGTCCAAGGACCCGGAGAAA | | |
| | CCTTCTGCTGGGCACTGCGTGTGCCATCTACTTGGGCTTCCTGGTGAGCCAGGTGGGG | | |
| | AGGGCCTCTCTCCAGCATGGACAGGCGGCTGAGAAGGGGCCACATCGCAGCCGCGACA | | |
| | CCGCCGAGCCATCCTTCCCTGAGATACCCCTGGATGGTACCCTGGCCCCTCCAGAGTC | | |
| | CCAGGGCAATGGGTCCACTCTGCAGCCCAATGTGGTGTACATTACCCTACGCTCCGAG | | |
| | CGCAGCAAGCCGGCCAATATCCGTGGCACCGTGAAGCCCAAGCGCAGGAAAAAGCATG | | |
| | CAGTGGCATCGGCTGCCCCAGGGCAGGAGGCTTTGGTCGGACCATCCCTTCAGCCGCA | | |
| | GGAAGCGGCAAGGGAAGCTGATGCTGTAGCACCTGGGTACGCTCAGGGAGCAAACCTG | | |
| | GTTAAGATTGGAGAGCGACCCTGGAGGTTGGTGCGGGGTCCGGGAGTGCGAGCCGGGG | | |
| | GCCCAGACTTCCTGCAGCCCAGCTCCAGGGAGAGCAACATTAGGATCTACAGCGAGAG | | |
| | CGCCCCCTCCTGGCTGAGCAAAGATGACATCCGAAGAATGCGACTCTTGGCGGACAGC | | |
| | GCAGTGGCAGGGCTCCGGCCTGTGTCCTCTAGGAGCGGAGCCCGTTTGCTGGTGCTGG | | |
| | AGGGGGGCGCACCTGGCGCTGTGCTCCGCTGTGGCCCTAGCCCCTGTGGGCTTCTCAA | | |
| | GCAGCCCTTGGACATGAGTGAGGTGTTTGCCTTCCACCTAGACAGGATCCTGGGGCTC | | |
| | AACAGGACCCTGCCGTCTGTGAGCAGGAAAGCAGAGTTCATCCAAGATGCCCGCCCAT | | |
| | GCCCCATCATTCTTTGGGATGCATCTTTATCTTCAGCAAGTAATGACACCCATTCTTC | | |
| | TGTTAAGCTCACCTGGGGAACTTATCAGCAGTTGCTGAAACAGAAATGCTGGCAGAAT | | |
| | GGCCGAGTACCCAAGCCTGAATCAGGTTGTACTGAAATACATCATCATGAGTGGTCCA | | |
| | AGATGGCACTCTTTGATTTTTTGTTACAGATTTATAATCGCTTAGATACAAATTGCTG | | |
| | TGGATTCAGACCTCGCAAGGAAGATGCCTGTGTACAGAATGGATTGAGGCCAAAATGT | | |
| | GATGACCAAGGTTCTGCGGCTCTAGCACACATTATCCAGCGAAAGCATGACCCAAGGC | | |
| | ATTTGGTTTTTATAGACAACAAGGGTTTCTTTGACAGGAGTGAAGATAACTTAAACTT | | |
| | CAAATTGTTAGAAGGCATCAAAGAGTTTCCAGCTTCTGCAGTTTCTGTTTTGAAGAGC | | |
| | CAGCACTTACGGCAGAAACTTCTTCAGTCTCTGTTTCTTGATAAAGTGTATTGGGAAA | | |
| | GTCAAGGAGGTAGACAAGGAATTGAAAAGCTTATCGATGTAATAGAACACAGAGCCAA | | |
| | AATTCTTATCACCTATATCAATGCACACGGGGTCAAAGTATTACCTATGAATGAATGA | | |
| | CAAAAGAATCTTCTGGCTAGGGTGTTAGATATATTTATGCATTTTTGGTTTTGTTTTT | | |
| | AAATCAAGCACATCAACCTCAAGCCCGTTTAGCAATGAG | | |
| | ORF Start: ATG at 413 | | ORF Stop: TGA at 1970 |
| | SEQ ID NO:130 | 519 aa | MW at 57552.4 kD |
| NOV41a, | MTCPDKPGQLINWFICSLCVPRVRRLWSSRRPRTRRNLLLGTACAIYLGFLVSQVGRA | | |
| CG57814-01 Protein Sequence | | | |
| | SLQHGQAAEKGPHRSRDTAEPSFPEIPLDGTLAPPESQGNGSTLQPNVVYITLRSERS | | |
| | KPANIRGTVKPKRRKKHAVASAAPGQEALVGPSLQPQEAAREADAVAPGYAQGANLVK | | |
| | IGERPWRLVRGPGVRAGGPDFLQPSSRESNIRIYSESAPSWLSKDDIRRMRLLADSAV | | |
| | AGLRPVSSRSGARLLVLEGGAPGAVLRCGPSPCGLLKQPLDMSEVFAFHLDRILGLNR | | |
| | TLPSVSRKAEFIQDGRPCPIILWDASLSSASNDTHSSVKLTWGTYQQLLKQKCWQNGR | | |
| | VPKPESGCTEIHHHEWSKMALFDFLLQIYNRLDTNCCGFRPRKEDACVQNGLRPKCDD | | |
| | QGSAALAHIIQRKHDPRHLVFTDNKGFFDRSEDNLNFKLLEGIKEFPASAVSVLKSQH | | |
| | LRQKLLQSLFLDKVYWESQGGRQGIEKLIDVIEHRAKILITYINAHGVKVLPMNE | | |
| | SEQ ID NO:131 | 1740 bp | |
| NOV41b, | GGCAGCTGAAGTCAGGAAACCATGCATCACATTAGCAGGAGCCAACTGCAGACTTTAA | | |
| CG57814-02 DNA Sequence | | | |
| | ACTCCGTTCAACATGTGGATGCGGCAGAGAAATGACCTGTCCAGACAAGCCGGGGCAG | | |
| | CTCATAAACTGGTTCATCTGCTCCCTGTGCGTCCCGCGGGTGCGTAAGCTCTGGAGCA | | |
| | GCCGGCGTCCAAGGACCCGGAGAAACCTTCTGCTGGGCACTGCGTGTGCCATCTACTT | | |
| | GGGCTTCCTGGTGAGCCAGGTGGGGAGGGCCTCTCTCCAGCATGGACAGGCGGCTGAG | | |
| | AAGGGGCCACATCGCAGCCGCGACACCGCCGAGCCATCCTTCCCTGAGATACCCCTGG | | |
| | ATGGTACCCTGGCCCCTCCAGAGTCCCAGGGCAATGGGTCCACTCTGCAGCCCAATGT | | |
| | GGTGTACATTACCCTACGCTCCAAGCGCAGCAAGCCGGCCAATATCCGTGGCACCGTG | | |
| | AAGCCCAAGCGCAGGAAAAAGCATGCAGTGGCATCGGCTGCCCAAGGGCAGGAGGCTT | | |
| | TGGTCGGACCATCCCTTCAGCCGCAAGAAGCGGCAAGGGAAGCTGATGCTGTAGCACT | | |
| | GGGTACGCTCAGGAGCAAACTGGTTAAGATGGAGAGCGACCCTGAAGGTGGTGCGGGG | | |
| | TCGGGAGTGCGAGCCGGGGGCCCAGACTTCCTGCAGCCCAGCTCCAGGGAGAGCAACA | | |
| | TTAGGATCTACAGCGAGAGCGCCCCCTCCTGGCTGAGCAAAGATGACATCCGAAGAAT | | |
| | GCGACTCTTGGCGGAGAGCGCAGTGGCAGGGCTCCGGCCTGTGTCCTCTAGGAGCGGA | | |
| | GCCCGTTTGCTGGTGCTGGAGGGGGGCGCACCTGGCGCTGTGCTCCGCTGTGGCCCTA | | |
| | GCCCCTGTGGGCTTCTCAAGCAGCCCTTGGACATGAGTGAGGTGTTTGCCTTCCACCT | | |
| | AGACAGGATCCTGGGGCTCAACAGGACCCTGCCGTCTGTGAGCAGGAAAGCAGAGTTC | | |
| | ATCCAAGATGGCCGCCCATGCCCCATCATTCTTTGGCATGCATCTTTATCTTCAGCAA | | |
| | GTAATGACACCCATTCTTCTGTTAAGCTCACCTGGGGAACTTATCAGCAGTTGCTGAA | | |
| | ACAGAAATGCTGGCAGAATGGCCGAGTACCCAAGCCTGAATCAGGTTGTACTGAAATA | | |
| | CATCATCATGAGTGGTCCAAGATGGCACTCTTTGATTTTTTGTTACAGATTTATAATC | | |
| | GCTTAGATACAAATTGCTGTGGATTCAGACCTCGCAACGAAGATGCCTGTGTACAGAA | | |
| | TGGATTGAGGCCAAAATGTGATGACCAAGGTTCTGCGGCTCTAGCACACATTATCCAG | | |
| | CGAAAGCATGACCCAAGGCATTTGGTTTTTATAGACAACAAGGGTTTCTTTGACAGGA | | |
| | GTGAAGATAACTTAAACTTCAAATTGTTAGAAGGCATCAAAGAGTTTCCAGCTTCTGC | | |
| | AGTTTCTGTTTTGAAGAGCCAGCACTTACGGCAGAAACTTCTTCAGTCTCTGTTTCTT | | |
| | GATAAAGTGTATTGGGAAAGTCAAGGAGGTAGACAAGGAATTGAAAAGCTTATCGATG | | |
| | TAATAGAACACAGAGCCAAAATTCTTATCACCTATATCAATGCACACGGGGTCAAAGT | | |
| | ATTACCTATGAATGAATGACAAAAGAATCTTCTGGCTAGCGTGTTAGATATATTTATG | | |
| | CATTTTTGGTTTTGTTTTTAAATCAAGCACATCAACCTCAAGCCCGTTTAGCAATGAG | | |
| | ORF Start: ATG at 90 | | ORF Stop: TGA at 1641 |
| | SEQ ID NO:132 | 517 aa | MW at 57179.9 kD |

| | |
|---|---|
| NOV41b, | MTCPDKPGQLINWFICSLCVPRVRKLWSSRRPRTRRNLLLGTACAIYLGFLVSQVGRA |
| CG57814-02 Protein Sequence | |
| | SLQHGQAAEKGPHRSRDTAEPSFPEIPLDGTLAPPESQGNGSTLQPNVVYITLRSKRS |
| | KPANIRGTVKPKRRKKHAVASAAQGQEALVGPSLQPQEAAREADAVALGTLRSKLVKM |
| | ESDPEGGAGSGVRAGGPDFLQPSSRESNIRIYSESAPSWLSKDDIRRMRLLADSAVAG |
| | LRPVSSRSGARLLVLEGGAPGAVLRCGPSPCGLLKQPLDMSEVFAFHLDRILGLNRTL |
| | PSVSRKAEFIQDGRPCPIILWDASLSSASNDTHSSVKLTWGTYQQLLKQKCWQNGRVP |
| | KPESGCTEIHHHEWSKMALFDFLLQIYNRLDTNCCGFRPRKEDACVQNGLRPKCDDQG |
| | SAALAHIIQRKHDPRHLVFIDNKGFFDRSEDNLNFKLLEGIKEFPASAVSVLKSQHLR |
| | QKLLQSLFLDKVYWESQGGRQGIEKLIDVIEHRAKILITYINAHGVKVLPMNE |

Sequence comparison of the above protein sequences yields the following sequence relationships shown in Table 41B.

TABLE 41B
Comparison of NOV41a against NOV41b.

| | | Identities/ |
|---|---|---|
| | NOV41a Residues/ | Similarities for |
| Protein Sequence | Match Residues | the Matched Region |
| NOV41b | 1 . . . 519 | 493/519 (94%) |
| | 1 . . . 517 | 497/519 (94%) |

Further analysis of the NOV41a protein yielded the following properties shown in Table 41C.

TABLE 41C
Protein Sequence Properties NOV41a

| | |
|---|---|
| PSort | 0.5500 probability located in endoplasmic reticulum |
| analysis: | (membrane); 0.2404 probability located in lysosome |
| | (lumen); 0.1000 probability located in endoplasmic |
| | reticulum (lumen); 0.1000 probability located in outside |
| SignalP | Likely cleavage site between residues 59 and 60 |
| analysis: | |

A search of the NOV41a protein against the Geneseq database, a proprietary database that contains sequences published in patents and patent publication, yielded several homologous proteins shown in Table 41D.

TABLE 41D
Geneseq Results for NOV41a

| | | NOV41a | Identities/ | |
|---|---|---|---|---|
| | | Residues/ | Similarities for | |
| Geneseq | Protein/Organism/Length | Match | the Matched | Expect |
| Identifier | [Patent #, Data] | Residues | Region | Value |
| AAU12276 | Human PRO6001 polypeptide | 1 . . . 519 | 518/519 (99%) | 0.0 |
| | sequence - Homo sapiens, 519 aa. | 1 . . . 519 | 519/519 (99%) | |
| | [WO200140466-A2, 7 JUN | | | |
| | 2001] | | | |
| AAM39125 | Human polypeptide SEQ ID NO | 1 . . . 519 | 518/519 (99%) | 0.0 |
| | 2270 - Homo sapiens, 519 aa. | 1 . . . 519 | 519/519 (99%) | |
| | [WO200153312-A1, 26 JUL | | | |
| | 2001] | | | |
| AAM40911 | Human polypeptide SEQ ID NO | 1 . . . 519 | 491/527 (93%) | 0.0 |
| | 5842 - Homo sapiens, 537 aa. | 19 . . . 537 | 495/527 (93%) | |
| | [WO200153312-A1, 26 JUL | | | |
| | 2001] | | | |
| AAM41373 | Human polypeptide SEQ ID NO | 212 . . . 512 | 130/316 (41%) | 1e−64 |
| | 6304 - Homo sapiens, 479 aa. | 161 . . . 471 | 180/316 (56%) | |
| | [WO200153312-A1, 26 JUL | | | |
| | 2001] | | | |
| AAM39587 | Human polypeptide SEQ ID NO | 212 . . . 512 | 130/316 (41%) | 1e−64 |
| | 2732 - Homo sapiens, 397 aa. | 79 . . . 389 | 180/316 (56%) | |
| | [WO200153312-A1, 26 JUL 2001] | | | |

In a BLAST search of public sequence databases, the NOV41a protein was found to have homology to the proteins shown in the BLASTP data in Table 41E.

TABLE 41E
Public BLASTP Results for NOV41a

| | | NOV41a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9ET25 | HYPOTHETICAL BASIC | 1 . . . 519 | 431/519 | (83%) | 0.0 |
| | PROTEIN I-19 - Mus musculus | 1 . . . 517 | 462/519 | (88%) | |
| | (Mouse), 517 aa. | | | | |
| Q9NYZ0 | AD021 PROTEIN - Homo | 274 . . . 519 | 246/246 | (100%) | e−145 |
| | sapiens (Human), 246 aa. | 1 . . . 246 | 246/246 | (100%) | |
| Q9UFP1 | HYPOTHETICAL 49.5 KDA | 212 . . . 512 | 129/316 | (40%) | 2e−63 |
| | PROTEIN - Homo sapiens | 130 . . . 440 | 179/316 | (55%) | |
| | (Human), 448 aa (fragment). | | | | |

PFam analysis predicts that the NOV41a protein contains the domains shown in the Table 41F.

TABLE 41F
Domain Analysis of NOV41a

| | | Identities/ | | |
|---|---|---|---|---|
| | | Similarities | | |
| | NOV41a | for the | | Expect |
| Pfam Domain | Match Region | Matched Region | | Value |
| SQS_PSY: | 109 . . . 145 | 8/37 | (22%) | 9.9 |
| domain 1 of 1 | | 29/37 | (78%) | |

### Example 42

The NOV42 clone was analyzed, and the nucleotide and predicted polypeptide sequences are shown in Table 42A.

TABLE 42A
NOV42 Sequence Analysis

| | SEQ ID NO:133 | 1294 bp | |
|---|---|---|---|
| NOV42a, | GATGGCCACCTTGAACGTTGTACTGATGTTGATGCCCCTTGCCCAGTACATTTTCCAT | | |
| CG59327-01 DNA Sequence | | | |
| | TGTTTTATAACTGTGCTACTGAAGTACTTGTGTGCAGAGTATGGCTGGAGGAATGCCA | | |
| | TGTTGATCCAAGICCCCGTTTCCTTAAACCTGTTTGTTTTTGGGACCCTCATGAGGCC | | |
| | CCTCCCTCCTGGGAAAAACCCAAATGACCCAGAAGAGAAAGATCTGCGCGTCCTGCCC | | |
| | GCGCACTCCACAGAGTCTGTAATGTCAAATGGACAGCAGGGAAGAATAGAAGAGAAGG | | |
| | ATGGCGGGTCTGGGAACGAGGAGACCCTCTGTGACCTGCAAGCCCAGGAGTGCAAGCC | | |
| | CAGGAGTGCCCCGATCAGGCCAGATCATGTGCGCTTTCCGGTTCTGAAGACGGTCAGC | | |
| | TGGCTCATTATGAGAGTCAAGAAGGGCTTCGAGGATTGGTACTCAGGCTATTTTGGGA | | |
| | CAGCCAGCCTATTTACAAATCGAATGTTTGTAGCCTTTGTTTTCTGGGCTTCATTTGC | | |
| | ATACAGCAGCTTTGTCATCTCCTTTATTCATCTCCCAGAAATCGTCAATTTGTATAAC | | |
| | TTATTGGAGCAAACGAAGGTTTTCCCTCTGACTTCAATTATAGCAATAGTTCACATTG | | |
| | TTGGAAAAGTGATCCTGGGCGTCATAGCTGACTTACCTTGCATCAGTGTTTGGAATGT | | |
| | CTTCCTGTTGGCCAGCTTCGTTCTTGTCCTCAGTATTTTTGTTTTGCTGCCTTTGATG | | |
| | CATATGTACGCTGGCCTGGTGGTCATCTGCACACTGACAGGGTTTTCCAGCGGTTATT | | |
| | TCTCCCTAATGCCCATAGTGACTGAAGACTTGGTTGGCATTGAACATTTGGCCAATGC | | |
| | CTACGGCATCATCATCTGTGCTAATGGCATCTCTGCGTTGTTGGGACCACCTTTTGCA | | |
| | GGTAAACTGTCTGAGGTTTTAAGAGTTCATAGTGCATATAGATACGGTGTGTTAGCTC | | |
| | TGCGAGGAGACGGATGCAGAGCACTCACATCTTCTCTTATACATAGAAGTGAAATGGC | | |
| | TTTCTAAAGTTAGATCACTGGCCAGAGTTTTTGAGTCACAAGAGCTATTCCACAGATT | | |
| | TCCTTTAGAAAAACAATCACCACTGGCAGTCCACTTCAGTGACACAGAATGGGTTGCA | | |
| | GAACTTGCTTACTTATGTGACACATTCAACCTGCTCAATGAACTCAATCTGTCACTTC | | |
| | AGGGGAGAAGGACAACTGTGTTCAAGTCAGCAAATAAAGTGGCTACATTCAAAACCAA | | |
| | ACTGGAATTACGGGGGTG | | |
| | ORF Start: ATG at 2 | | Stop: TAA at 1049 |
| | SEQ ID NO:134 | 349 aa | MW at 38694.2 kD |

| | |
|---|---|
| NOV42a, | MATLNVVLMLNPLAQYIFHCFITVLLKYLCAEYGWRNAMLIQGAVSLNLFVFGTLMRP |
| CG59327-01 Protein Sequence | |
| | LPPGKNPNDPEEKDLRVLPAHSTESVNSNGQQGRIEEKDGGSGNEETLCDLQAQECKP |
| | RSAPIRPDHVRFPVLKTVSWLIMRVKKGFEDWYSGYFGTASLFTNRMFVAFVFWASFA |
| | YSSFVISFIHLPEIVNLYNLLEQTKVFFLTSIIAIVHIVGKVILGVIADLPCISVWNV |
| | FLLASFVLVLSIFVLLPLMHMYAGLVVICTLTGFSSGYFSLMPIVTEDLVGIEHLANA |
| | YGIIICANGISALLGPPFAGKLSEVLRVHSAYRYGVLALRGDGCRALTSSLIHRSEMAF |

Further analysis of the NOV42a protein yielded the following properties shown in Table 42B.

TABLE 42B
Protein Sequence Properties NOV42a

| | |
|---|---|
| PSort | 0.6850 probability located in endoplasmic reticulum |
| analysis: | (membrane); 0.6400 probability located in plasma |
| | membrane; 0.4600 probability located in Golgi |
| | body; 0.1000 probability located in endoplasmic |
| | reticulum (lumen) |
| SignalP | Likely cleavage site between residues 32 and 33 |
| analysis: | |

A search of the NOV42a protein against the Geneseq database, a proprietary database that contains sequences published in patents and patent publication, yielded several homologous proteins shown in Table 42C.

TABLE 42C
Geneseq Results for NOV42a

| | | NOV42a | Identities/ | | |
|---|---|---|---|---|---|
| | | Residues/ | Similarities | | |
| Geneseq | Protein/Organism/Length | Match | for the | | Expect |
| Identifier | [Patent #, Data] | Residues | Matched Region | | Value |
| AAO07132 | Human polypeptide SEQ ID NO | 257 . . . 331 | 49/77 | (63%) | 6e−20 |
| | 21024 - Homo sapiens, 107 aa. | 5 . . . 81 | 58/77 | (74%) | |
| | [WO200164835-A2, 7 SEP 2001] | | | | |
| AAY31642 | Human transport-associated protein- | 157 . . . 342 | 54/197 | (27%) | 1e−07 |
| | 4 (TRANP-4) - Homo sapiens, 465 | 221 . . . 401 | 86/197 | (43%) | |
| | aa. [WO9941373-A2, 19 AUG | | | | |
| | 1999] | | | | |
| AAY02737 | Human secreted protein encoded by | 198 . . . 342 | 41/147 | (27%) | 9e−06 |
| | gene 88 clone HKAFB88 - Homo | 24 . . . 164 | 65/147 | (43%) | |
| | sapiens, 229 aa. [WO9902546-A1, | | | | |
| | 21 JAN 1999] | | | | |

In a BLAST search of public sequence databases, the NOV42a protein was found to have homology to the proteins shown in the BLASTP data in Table 42D.

TABLE 42D
Public BLASTP Results for NOV42a

| | | | Identities/ | | |
|---|---|---|---|---|---|
| Protein | | | Similarities | | |
| Accession | | NOV42a Residues/ | for the | | Expect |
| Number | Protein/Organism/Length | Match Residues | Matched Portion | | Value |
| Q96NI7 | CDNA FLJ30794 FIS, CLONE | 22 . . . 331 | 250/312 | (80%) | e−138 |
| | FEBRA2001093, WEAKLY | 1 . . . 310 | 266/312 | (85%) | |
| | SIMILAR TO | | | | |
| | MONOCARBOXYLATE | | | | |
| | (Human), 336 aa. | | | | |
| Q9D1K0 | 1110004H10RIK PROTEIN - Mus | 22 . . . 331 | 220/312 | (70%) | e−119 |
| | musculus (Mouse), 336 aa. | 1 . . . 310 | 250/312 | (79%) | |
| AAL39716 | LD30953P - Drosophila | 142 . . . 314 | 50/180 | (27%) | 2e−15 |
| | melanogaster (Fruit fly), 894 aa. | 665 . . . 843 | 89/180 | (48%) | |
| Q9V9B3 | CG3409 PROTEIN - Drosophila | 142 . . . 314 | 50/180 | (27%) | 2e−15 |
| | melanogaster (Fruit fly), 800 aa. | 571 . . . 749 | 89/180 | (48%) | |
| Q9W0L6 | CG13907 PROTEIN - Drosophila | 157 . . . 331 | 55/178 | (30%) | 1e−14 |
| | melanogaster (Fruit fly), 816 aa. | 565 . . . 738 | 91/178 | (50%) | |

PFam analysis predicts that the NOV42a protein contains the domains shown in the Table 42E.

TABLE 42E
Domain Analysis of NOV42a

| | | Identities/ | |
|---|---|---|---|
| | | Similarities | |
| | NOV42a | for the | Expect |
| Pfam Domain | Match Region | Matched Region | Value |
| oxidored_q3: | 197 . . . 314 | 25/177 (14%) | 9.1 |
| domain 1 of 1 | | 73/177 (41%) | |

### Example 43

The NOV43 clone was analyzed, and the nucleotide and predicted polypeptide sequences are shown in Table 43A.

TABLE 43A
NOV43 Sequence Analysis

| | SEQ ID NO:135 | 455 bp | |
|---|---|---|---|
| NOV43a, | TAGAACTGTGTTGAGCTCTCACCCATCACGATGAGCAACAAATTCTTGGGAACCTGGA | | |
| CG59494-01 DNA Sequence | | | |
| | AGCTGGTCTCCAGTGAAAACTTTGAGGATTACATGAAAGAACTGGGAGTGAATTTCGC | | |
| | AGCCCGGAACATGGCAGGGTTAGTGAAACCGACAGTAACTATTAGTGTTGATGGGAAA | | |
| | ATGATGACCATAAGAACAGAAAGTTCTTTCCAGGACACTAAGATCTCCTTCAAGCTGG | | |
| | GGGAAGAATTTGATGAAACTACAGCAGACAACCGGAAAGTAAAGAGCACCATAACATT | | |
| | AGAGAATGGCTCAATGATTCACGTCCAAAAATGGCTTGGCAAAGAGACAACAATCAAA | | |
| | AGAAAAATTGTGGATGAAAAAATGGTAGTGGAATGTAAAATGAATAATATTGTCAGCA | | |
| | CCAGAATCTACGAAAAGGTCTGAAAAATCATTTCTTCATTGAAGTGGCT | | |
| | ORF Start: ATG at 31 | | ORF Stop: TGA at 427 |
| | SEQ ID NO:136 | 132 aa | MW at 15096.4 kD |

| | |
|---|---|
| NOV43a, | MSNKFLGTWKLVSSENFEDYMKELGVNFAARNMAGLVKPTVTISVDGKMMTIRTESSF |
| CG59494-01 Protein Sequence | |
| | QDTKISFKLGEEPDETTADNRRVKSTITLENGSMIHVQKWLGKETTIKRKIVDEKMVV |
| | ECKMNNIVSTRIYEKV |

Further analysis of the NOV43a protein yielded the following properties shown in Table 43B.

TABLE 43B
Protein Sequence Properties NOV43a

| | |
|---|---|
| PSort | 0.6500 probability located in cytoplasm; 0.1000 |
| analysis: | probability located in mitochondrial matrix space; |
| | 0.1000 probability located in lysosome (lumen); |
| | 0.0053 probability located in microbody (peroxisome) |
| SignalP | No Known Signal Sequence Predicted |
| analysis: | |

A search of the NOV43a protein against the Geneseq database, a proprietary database that contains sequences published in patents and patent publication, yielded several homologous proteins shown in Table 43C.

TABLE 43C
Geneseq Results for NOV43a

| | | NOV43a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAW40227 | Human myelin P2 protein-Homo | 1 . . . 130 | 89/130 (68%) | 2e−47 |
| | sapiens, 136 aa. [WO9803647-A2, | 1 . . . 130 | 107/130 (81%) | |
| | 29 JAN. 1998] | | | |
| AAW40228 | Bovine myelin P2 protein-Bos | 1 . . . 130 | 89/130 (68%) | 9e−47 |
| | taurus, 136 aa. [WO9803647-A2, | 1 . . . 130 | 106/130 (81%) | |
| | 29 JAN. 1998] | | | |
| AAY90320 | Human AFABP protein sequence- | 1 . . . 131 | 84/131 (64%) | 3e−46 |
| | Homo sapiens, 132 aa. | 1 . . . 131 | 110/131 (83%) | |
| | [WO200047734-A1, | | | |
| | 17 AUG. 2000] | | | |
| AAY90319 | Mouse AFABP protein sequence- | 1 . . . 131 | 83/131 (63%) | 7e−45 |
| | Mus sp, 132 aa. [WO200047734- | 1 . . . 131 | 108/131 (82%) | |
| | A1, 17 AUG. 2000] | | | |
| AAG66576 | Mouse MDGI polypeptide-Mus | 1 . . . 131 | 73/131 (55%) | 6e−40 |
| | sp, 133 aa. [U.S. Pat. | 1 . . . 131 | 103/131 (77%) | |
| | No. 6232291-B1, 15 MAY 2001] | | | |

In a BLAST search of public sequence databases, the NOV43a protein was found to have homology to the proteins shown in the BLASTP data in Table 43D.

TABLE 43D
Public BLASTP Results for NOV43a

| | | NOV43a | | |
|---|---|---|---|---|
| Protein | | Residues/ | Identities/ | |
| Accession | | Match | Similarities for | Expect |
| Number | Protein/Organism/Length | Residues | the Matched Portion | Value |
| MPRB2 | myelin P2 protein-rabbit, 132 aa. | 1 . . . 132 | 95/132 (71%) | 3e−49 |
| | | 1 . . . 132 | 109/132 (81%) | |
| P02691 | Myelin P2 protein-Oryctolagus | 2 . . . 132 | 94/131 (71%) | 1e−48 |
| | cuniculus (Rabbit), 131 aa. | 1 . . . 131 | 108/131 (81%) | |
| MPHU2 | myelin P2 protein [validated]- | 1 . . . 132 | 92/132 (69%) | 3e−48 |
| | human, 132 aa. | 1 . . . 132 | 109/132 (81%) | |
| Q90X56 | ADIPOCYTE FATTY ACID | 1 . . . 131 | 86/131 (65%) | 1e−47 |
| | BINDING PROTEIN-Gallus | 1 . . . 131 | 113/131 (85%) | |
| | gallus (Chicken), 132 aa. | | | |
| P02689 | Myelin P2 protein-Homo sapiens | 2 . . . 132 | 91/131 (69%) | 1e−47 |
| | (Human), 131 aa. | 1 . . . 131 | 108/131 (81%) | |

PFam analysis predicts that the NOV43a protein contains the domains shown in the Table 43E.

TABLE 43E
Domain Analysis of NOV43a

| | | Identities/ | |
|---|---|---|---|
| Pfam | NOV43a | Similarities for | Expect |
| Domain | Match Region | the Matched Region | Value |
| lipocalin: domain 1 of | 4 . . . 132 | 45/157 (29%) | 3.2e−36 |
| 1 | | 113/157 (72%) | |

### Example 44

The NOV44 clone was analyzed, and the nucleotide and predicted polypeptide sequences are shown in Table 44A.

TABLE 44A
NOV44 Sequence Analysis

| | SEQ ID NO:137 | 1561 bp | |
|---|---|---|---|
| NOV44a, | AAGAATTGTAGCTCTCCACTGAATTGCAGGGGTTCTTGAATGTTGTCAACATTTGGAG | | |
| CG59432-01 DNA Sequence | | | |
| | GCAGTTGGAGGAGGGAGCTCTATTGATGAAAAATGGCTACATATTCAAAATTTCAGTG | | |
| | TATACCAGGAAGATAATTCAATTCAATCTCTGGCTTACCCAAAGAATCTTGGAGTTAC | | |
| | TGCCAATGAGGAAATCCCCAGGGTCTAATAAAAATATCTTTAGGAGTGAAGGAGTTAA | | |
| | CTGAGTGTGTAAGCTTTATCTTCTGTCCAATGGACTTGTGGTTTGCTTATAAAACTCT | | |
| | CCAGTAAATAATTGTTAGAGACCTGTCATTGATAGCAGTTGCTAGTTGCTGCCTTTTA | | |
| | AGAGCTCGTTGATTCCTCTGCAAGGTGGTGCAGCATCCTCTGTCCCTTCATTCATTTC | | |
| | AGATCTACTCAGGTCTCCCTGTAAACAGATCTCTCGGATCAATAAGCATGAATGACGA | | |
| | AGACTACACCACCATCTATGACACAATCCAAAATGAGAGGACGTATGAGGTTCCAGAC | | |
| | CAGCCAGAAGAAAATGAAAGTCCCCATTATGATGATGTCCATGAGTACTTAAGGCCAG | | |
| | AAAATGATTTATATGCCACTCAGCTGAATACCCATGAGTATGATTTTGTGTCAGTCTA | | |
| | TACCATTAAGGGTGAAGAGACCAGCTTGGCCTCTGTCCAGTCAGAAGACAGAGGCTAC | | |
| | CTCCTGCCTGATGAGATATACTCTGAACTCCAGGAGGCTCATCCAGGTGAGCCCCAGG | | |
| | AGGACAGGGGCATCTCAATGGAAGGGTTATATTCATCAACCCAGGACCAGCAACTCTG | | |
| | CGCAGCAGAACTCCAGGAGAATGGGAGTGTGATGAAGGAAGATCTGCCTTCTCCTTCA | | |
| | AGCTTCACCATTCAGCACAGTAAGGCCTTCTCTACCACCAAGTATTCCTGCTATTCTG | | |
| | ATGCTGAAGGTTTGGAAGAAAAGGAGGCAGCTCACATGAACCCTGAGATTTACCTCTT | | |
| | TGTGAAGGTAAGGTCTGCCTCTGACAGGCATACCCTGTTCATGCAGATATTATGGCTG | | |
| | GTGTTTTATTTTGCTCTGAATGACCAGGGAAAGATTCATAATGCCATGGTCCTTGGAT | | |
| | CTCAATACATATTCAGGAGTCGGAGGGACTAAATCAGTCATTAGAGTGTACTCAGCTC | | |
| | TTCACAAAATTAGAGGAATTGGAAGGTGCATTTAAAGCACGTATTTAATCACTGACTT | | |
| | TTACATACCATGGGCAAAGTATTTTTCAAAACGGTTCACATAAGTGAGCCATAACTGC | | |
| | TGCCCAAATCCTTGCCATTGTGGCTGACATTAAGTACATTTTTCTGTCTGGTTAAATT | | |
| | TCCTTTGTCGACATGTTTAAAAGTGAAACCAAAGCTTGTGAAAGAAAGACCTTCTTGT | | |
| | GCTTCTAAGGTCACAGATTTGTCAGATAGGTGGTCAATAAAGGCTATCTCTGTCACTA | | |
| | GCTTGCCCCTTTGGCACCAATATAACTAAAAATTTGATGAAGTCAAATGATTTCAGTA | | |
| | GTAGTAAGACACTACCAGTGTTAATGTTTAATACTTACGATATCTAAACAGAA | | |
| | ORF Start: ATG at 454 | | ORF Stop: TAA at 1132 |
| | SEQ ID NO:138 | 226 aa | MW at 26132.2 kD |
| NOV44a, | MNDEDYSTIYDTIQNERTYEVPDQPEENESPHYDDVHEYLRPENDLYATQLNTHEYDF | | |
| CG59432-01 Protein Sequence | | | |
| | VSVYTIKGEETSLASVQSEDRGYLLPDEIYSELQEAHPGEPQEDRGISMEGLYSSTQD | | |
| | QQLCAAELQENGSVMKEDLPSPSSFTIQHSKAFSTTKYSCYSDAEGLEEKEGAHMNPE | | |
| | IYLFVKVRSASDRHTLFMQILWLVFYFALNDQGKIHNAMVLGSQYIFRSRRD | | |
| | SEQ ID NO:139 | 809 bp | |
| NOV44b, | ATCCTCTGTCCCTTCATTCATTTCAGATCTACTCAGGTCTCCCTGTAAACAGATCTCT | | |
| CG59432-02 DNA Sequence | | | |
| | CGGATCAATAAGCATGAATGACGAAGACTACGGCACCATCTATGACACAATCCAAAAT | | |
| | GAGAGGACGTATGAGGTTCCAGACCAGCCAGAAGAAAATGAAAGTCCCCATTATGATG | | |
| | ATGTCCATGAGTACTTAAGGCCAGAAAATGATTTATATGCCACTCAGCTGAATACCCA | | |
| | TGAGTATGATTTTGTGTCAGTCTATACCATTAAGGGTGAAGAGACCAGCTTGGCCTCT | | |
| | GTCCAGTCAGAAGACAGAGGCTACCTCCTGCCTGATGAGATATACTCTGAACTCCAGG | | |
| | AGGCTCATCCAGGTGAGCCCCAGGAGGACAGGGGCATCTCAATGGAAGGGTTATATTC | | |
| | ATCAACCCAGGACCAGCAACTCTGCGCAGCAGAACTCCAGGAGAATGGGAGTGTGATG | | |
| | AAGGAAGATCTGCCTTCTCCTTCAAGCTTCACCATTCAGCACAGTAAGGCCTTCTCTA | | |
| | CCACCAAGTATTCCTGCTATTCTGATGCTGAAGGTTTGGAAGAAAAGGAGGGAGCTCA | | |
| | CATGAACCCTGAGATTTACCTCTTTGTGAAGGTAAGGTCTGCCTCTGACAGGCATACC | | |
| | CTGTTCATGCAGATATTATGGCTGCTGTTTTATTTTCCTCTGAATGACCAGGGAAAGA | | |
| | TTCATAATGCCATGGTCCTTGGATCTCAATACATATTCAGGAGTCGGAGGGACTAAAT | | |
| | CAGTCATTAGAGTGTACTCAGCTCTTCACAAAATTAGAGGAATTGGAAGGTGCAT | | |
| | ORF Start: ATG at 72 | | ORF Stop: TAA at 750 |
| | SEQ ID NO:140 | 226 aa | MW at 26102.2 kD |

| | |
|---|---|
| NOV44b, | MNDEDYGTIYDTIQNERTYEVPDQPEENESPHYDDVHEYLRPENDLYATQLNTHEYDF |
| CG59432-02 Protein Sequence | |
| | VSVYTIKGEETSLASVQSEDRGYLLPDEIYSELQEAHPGEPQEDRGISMEGLYSSTQD |
| | QQLCAAELQENGSVNKEDLPSPSSFTIQHSKAFSTTKYSCYSDAEGLEEKEGAHMNPE |
| | IYLFVKVRSASDRHTLFMQILWLVFYFALNDQGKIHNANVLGSQYIFRSRRD |

Sequence comparison of the above protein sequences yields the following sequence relationships shown in Table 44B.

TABLE 44B
Comparison of NOV44a against NOV44b.

| | | Identities/ |
|---|---|---|
| Protein | NOV44a Residues/ | Similarities for |
| Sequence | Match Residues | the Matched Region |
| NOV44b | 1 . . . 226 | 225/226 (99%) |
| | 1 . . . 226 | 225/226 (99%) |

Further analysis of the NOV44a protein yielded the following properties shown in Table 44C.

TABLE 44C
Protein Sequence Properties NOV44a

| | |
|---|---|
| PSort | 0.6500 probability located in cytoplasm; 0.1000 |
| analysis: | probability located in mitochondrial matrix space; |
| | 0.1000 probability located in lysosome (lumen); |
| | 0.0000 probability located in endoplasmic reticulum |
| | (membrane) |
| SignalP | No Known Signal Sequence Predicted |
| analysis: | |

A search of the NOV44a protein against the Geneseq database, a proprietary database that contains sequences published in patents and patent publication, yielded several homologous proteins shown in Table 44D.

TABLE 44D
Geneseq Results for NOV44a

| | | | | |
|---|---|---|---|---|
| | | NOV44a | | |
| | Protein/Organism/ | Residues/ | Identities/ | |
| Geneseq | Length [Patent #, | Match | Similarities for | Expect |
| Identifier | Date] | Residues | the Matched Region | Value |

No Significant Matches Found

In a BLAST search of public sequence databases, the NOV44a protein was found to have homology to the proteins shown in the BLASTP data in Table 44E.

TABLE 44E
Public BLASTP Results for NOV44a

| | | NOV44a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96JT5 | CLIC5B-Homo sapiens (Human), | 1 . . . 200 | 185/202 (91%) | e−104 |
| | 410 aa. | 1 . . . 202 | 191/202 (93%) | |
| Q9NPY9 | DJ447E21.4 (SIMILAR TO | 1 . . . 180 | 180/180 (100%) | e−103 |
| | BOVINE CHLORIDE CHANNEL | 1 . . . 180 | 180/180 (100%) | |
| | PROTEIN (P64))-Homo sapiens | | | |
| | (Human), 180 aa (fragment). | | | |
| A47104 | chloride channel 64K chain- | 1 . . . 197 | 104/231 (45%) | 1e−39 |
| | bovine, 437 aa. | 1 . . . 229 | 133/231 (57%) | |
| P35526 | Chlorine channel protein p64-Bos | 1 . . . 197 | 103/231 (44%) | 1e−38 |
| | taurus (Bovine), 437 aa. | 1 . . . 229 | 131/231 (56%) | |

PFam analysis predicts that the NOV44a protein contains the domains shown in the Table 44F.

TABLE 44F
Domain Analysis of NOV44a

| | | | |
|---|---|---|---|
| | | Identities/ | |
| Pfam | NOV44a | Similarities for | Expect |
| Domain | Match Region | the Matched Region | Value |

No Significant Matches Found

### Example 45

The NOV45 clone was analyzed, and the nucleotide and predicted polypeptide sequences are shown in Table 45A.

TABLE 45A
NOV45 Sequence Analysis

| | SEQ ID NO:141 | 877 bp | |
|---|---|---|---|
| NOV45a, | ACTTTGTCCTCTTGGGCTTCACACAGAATCCAAAGGAGCAGAAAGTACTTTTTGTTAT | | |
| CS59394-01 DNA Sequence | | | |
| | GTTCTTGCTCTTCTACATTTTGACCATGGTGGGCAACCTGCTCATTGTAGTGACCGTA | | |
| | ACTGTCAGTGACACCCTGGGCTCACCAATGTACTTCTTTCTTGCTGGCTTATCATTTA | | |
| | TAGATATCATTTATTCTTCATCCATTTCCCCCAGATTGATTTCAGGCTTGTTCTTTGG | | |
| | GAATAATTCCATATCCTTCCAATCTTGCATGGCCCAGCTCTTTATCGAGCACATTTTC | | |
| | GGTGGGTCAGAGGTCTTTCTCCTGTTGGTQATGGCCTATGACTGCTATGTGGCCATCT | | |
| | GTAAGCCCTTGCATTATTTGGTTATCATGAGACAATGGGTGTGTGTTCTGCTGCTGGT | | |
| | AGTGTCCTGGGTTGGAGGATTTCTGCACTCAGTATTTCAACTTAGCATTATTTATGGG | | |
| | CTCCCATTCTGTGGCCCCAATGTCATTGATCATTTTTTCTGTGACATGTATCCCTTAT | | |
| | TGAAACTGGTCTGCACTGACACCCATGCTATTGGCCTCTTAGTGGTOGCCAATGGAGG | | |
| | ACTGGCTTGCACTATTGTGTTTCTGCTCTTACTCATCTCTTATGGTGTCATCTTGCAC | | |
| | TCTTTAAAGAACCTTAGTCAGAAAGGGAGGCAAAAAGCCCTCTCAACCTGCAGTTCCC | | |
| | ACATGACTGTGGTTGTCTTCTTCTTTGTTCCTTGTATTTTTATGTATcCTAGACCTGC | | |
| | TAGGACCTTCCCCATTGACAAATCAGTGAGTGTGTTTTATACAGTCATAACCCCAATG | | |
| | CTGAACCCCTTAATCTACACTCTGAGAAATTCTGAGATGACAAGTGCTATGAAGAAGC | | |
| | TTTAGAG | | |
| | ORF Start: TTT at 3 | | ORF Stop: TAG at 873 |
| | SEQ ID NO:142 | 290 aa | MW at 32485.7 kD |

| | |
|---|---|
| NOV45a, | FVLLGFTQNPKEQKVLFVMFLLFYILTMVGNLLIVVTVTVSETLGSPMYFFLAGLSFI |
| CG59394-01 Protein Sequence | |
| | DIIYSSSISPRLISGLFFGNNSISFQSCMAQLFIEHIFGGSEVFLLLVMAYDCYVAIC |
| | KPLHYLVIMRQWVCVVLLVVSWVGGFLHSVFQLSIIYGLPFCGPNVIDHFFCDMYPLL |
| | KLVCTDTHAIGLLVVANGGLACTIVFLLLLISYGVILHSLKNLSQKGRQKALSTCSSH |
| | MTVVVFFFVPCIFMYARPARTFPIDKSVSVFYTVITPMLNPLIYTLRNSEMTSAMKKL |

Further analysis of the NOV45a protein yielded the following properties shown in Table 45B.

TABLE 45B
Protein Sequence Properties NOV45a

| | |
|---|---|
| PSort | 0.6400 probability located in plasma membrane; 0.4600 |
| analysis: | probability located in Golgi body; 0.3700 probability |
| | located in endoplasmic reticulum (membrane); |
| | 0.1000 probability located in endoplasmic reticulum (lumen) |
| SignalP | Likely cleavage site between residues 42 and 43 |
| analysis: | |

A search of the NOV45a protein against the Geneseq database, a proprietary database that contains sequences published in patents and patent publication, yielded several homologous proteins shown in Table 45C.

TABLE 45C
Geneseq Results for NOV45a

| | | NOV45a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU24536 | Human olfactory receptor | 1 . . . 290 | 273/290 (94%) | e−155 |
| | AOLFR21-Homo sapiens, 299 aa. | 10 . . . 299 | 278/290 (95%) | |
| | [WO200168805-A2, | | | |
| | 20 SEP. 2001] | | | |
| AAG71950 | Human olfactory receptor | 1 . . . 290 | 273/290 (94%) | e−155 |
| | polypeptide, SEQ ID NO: 1631- | 10 . . . 299 | 278/290 (95%) | |
| | Homo sapiens, 299 aa. | | | |
| | [WO200127158-A2, | | | |
| | 19 APR. 2001] | | | |
| AAG72258 | Human olfactory receptor | 33 . . . 290 | 234/258 (90%) | e−131 |
| | polypeptide, SEQ ID NO: 1939- | 1 . . . 250 | 240/258 (92%) | |
| | Homo sapiens, 262 aa. | | | |
| | [WO200127158-A2, | | | |
| | 19 APR. 2001] | | | |
| AAG72553 | Human OR-like polypeptide query | 1 . . . 290 | 198/290 (68%) | e−121 |
| | SEQ NO: 2234- | 10 . . . 299 | 242/290 (83%) | |
| | Homo sapiens, 327 aa. | | | |
| | [WO200127158-A2, | | | |
| | 19 APR. 2001] | | | |
| AAG71909 | Human olfactory receptor | 1 . . . 290 | 198/290 (68%) | e−121 |
| | polypeptide, SEQ ID NO: 1590- | 10 . . . 299 | 242/290 (83%) | |
| | Homo sapiens, 327 aa. | | | |
| | [WO200127158-A2, | | | |
| | 19 APR. 2001] | | | |

In a BLAST search of public sequence databases, the NOV45a protein was found to have homology to the proteins shown in the BLASTP data in Table 45D.

TABLE 45D
Public BLASTP Results for NOV45a

| | | NOV45a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9QW37 | OR18=ODORANT RECEPTOR- | 1 . . . 290 | 192/290 (66%) | e−115 |
| | Rattus sp, 307 aa. | 10 . . . 299 | 237/290 (81%) | |
| Q96R66 | OLFACTORY RECEPTOR- | 57 . . . 269 | 198/213 (92%) | e−111 |
| | Homo sapiens (Human), 213 aa | 1 . . . 213 | 202/213 (93%) | |
| | (fragment). | | | |
| Q9R0K2 | ODORANT RECEPTOR | 1 . . . 290 | 177/290 (61%) | e−105 |
| | MOR18-Mus musculus | 10 . . . 299 | 229/290 (78%) | |
| | (Mouse), 308 aa. | | | |
| Q9R0K1 | ODORANT RECEPTOR A16- | 1 . . . 290 | 171/290 (58%) | e−102 |
| | Mus musculus (Mouse), 302 aa. | 10 . . . 299 | 226/290 (76%) | |
| CAC88333 | SEQUENCE 34 FROM PATENT | 1 . . . 290 | 167/290 (57%) | 5e−99 |
| | WO0164879-Homo sapiens | 10 . . . 299 | 221/290 (75%) | |
| | (Human), 309 aa. | | | |

PFam analysis predicts that the NOV45a protein contains the domains shown in the Table 45E.

TABLE 45E
Domain Analysis of NOV45a

| | | Identities/ | |
|---|---|---|---|
| Pfam | NOV45a | Similarities for | Expect |
| Domain | Match Region | the Matched Region | Value |
| 7tm_1: domain 1 of 1 | 30 . . . 276 | 50/268 (19%) | 4.4e−23 |
| | | 174/268 (65%) | |

### Example 46

The NOV46 clone was analyzed, and the nucleotide and predicted polypeptide sequences are shown in Table 46A.

TABLE 46A
NOV46 Sequence Analysis

| | SEQ ID NO:143 | 1746 bp | |
|---|---|---|---|
| NOV46a, | ATAATTCAGTTTGAAAACCAGTGGTTTCTCTTTCCTTCCCTATAGGTGTAAAGAATAT | | |
| CG59383-01 DNA Sequence | | | |
| | CCAGCTGGTGGCTACAGTTCCCCCTCTGGTTTTGCTGCCATGCATCCTGGGCGAACTA | | |
| | CTGGTAAAGGGCCCTCTACTCACACTCAGATTGACCAGCAACCTCCACGGCTTCTCAT | | |
| | TGTGCACATTGCTCTACCGTCCTGGGCTGACATCTGCACCAACCTCTGTGAGGCTCTG | | |
| | CAGAACTTCTTCTCTCTAGCCTGCAGCTTGATGGGCCCCAGCCGCATGTCCCTGTTCA | | |
| | GTTTATACATGGTACAAGATCAGCATGAGTGCATCCTCCCTTTTGTGCAAGTGAAAGG | | |
| | GAACTTTGCTAGOTTGCAGACCTGCATCTCAGAACTCCGCATGTTACAGAGAGAAGGG | | |
| | TGTTTCAGATCACAAGGTGCTTCTCTGCGGCTGGCAGTAGAGGATGGGCTCCAGCAAT | | |
| | TCAAACAATACAGCAGACATGTGACCACAAGGGCAGCTCTGACCTATACCTCCCTGGA | | |
| | GATTACTATTCTGACTTCTCAGCCTGGAAAAGAGGTGGTCAAACAGTTGGAGGAAGGG | | |
| | TTGAAAGATACAGACCTAGCCAGAGTCAGGAGGTTTCAGGTCGTTGAGGTCACAAAGG | | |
| | GAATCCTAGAGCACGTGGACTCAGCGTCTCCTGTTGAGGATACCAGCAATGATGAGAG | | |
| | TTCTATTCTGGGAACTGACATTGACCTTCAGACTATAGACAATGATATCGTCAGCATG | | |
| | GAGATTTTCTTCAAAGCCTGGCTACATAACAGTGGAACAGACCAAGAACAAATCCATC | | |
| | TTCTTCTTTCTTCACAGTGTTTCAGCAACATTTCCAGACCCAGAGATAATCCAATGTG | | |
| | TCTGAAATGTGATCTCCAAGAGCGACTGCTCTGCCCATCCCTACTCGCTGGCACAGCT | | |
| | GACGGCTCCTTGAGAATGGATGACCCTAAAGGAGACTTCATCACACTCTACCAGATGG | | |
| | CTTCCCAGTCATCGGCCTCTCATTACAAOCTCCAAGTGATCAAGGCTTTAAAATCTAG | | |
| | CGGGCTCTGCGAGTCATTGACATATGGACTCCCGTTCATCCTCAGACCTACAAGCTGT | | |
| | TGGCAGCTCGACTGGGATGAGCTGGAGACAAATCAGCAACATTTCCATGCTTTGTGTC | | |
| | ACAGCCTGCTGAAAAGGGAATGGCTGCTGTTAGCCAAGGGGGAACCACCGGGCCCAGG | | |
| | ACACAGCCAGAGAATTCCTGCCAGCACCTTCTATGTGATCATGCCGTCACACTCCCTC | | |
| | ACACTGCTGGTAAAGGCGGTGGCCACGCGGGAACTGATGCTGCCCAGCACCTTCCCCC | | |
| | TGCTACCTGAGGACCCACATGATGATAGCCTTAAGAATAGCATGCTGGACAGCCTGGA | | |
| | GCTGGAGCCCACCTACAACCCCTTGCATGTTCAAAGCCACCTGTACTCACACCTGAGC | | |
| | AGCATCTATGCCAAGCCTCAGGGGCGGCTCCACCCACACTGGGAGAGCCGAGCTCCGA | | |
| | GAAAGACTGGGCAGTTGCAGACCAACCGAGCTCGAGCTACTGTGGCCCCCCTGCCTAT | | |
| | GACTCCTGTCCCAGGCAGAGCCTCCAAGATGCCAGCAGCCAGCAAATCTTCCTCAGAT | | |
| | GCCTTCTTCCTGCCTTCAGAGTGGGAGAAGGATCCCTCAAGGCCCTAAGTCACCAGCA | | |
| | CCAGAGCCCAGCTGCCCAGCTTAACCATATCCATGCTCAGGTTCACATAATGGCTATC | | |
| | TGTGGT | | |
| | ORF Start: ATG at 98 | | ORF Stop: TAA at 1670 |
| | SEQ ID NO:144 | 524 aa | MW AT 58691.3 kD |
| NOV46a, | MHPGRTTGKGPSTHTQIDQQPPRLLIVHIALPSWADICTNLCEALQNFFSLACSLMGP | | |
| CG59383-01 Protein Sequence | | | |
| | SRMSLFSLYMVQDQHECILPFVQVKGNFARLQTCISELRMLQREGCFRSQGASLRLAV | | |
| | EDGLQQEKQYSRHVTTRAALTYTSLEITILTSQPGKEVVKQLEEGLKDTDLARVRRFQ | | |
| | VVEVTKGILEHVDSASPVEDTSNDESSILGTDIDLQTIDNDTVSMEIFFKAWLHNSGT | | |
| | DQEQIHLLLSSQCFSNISRPRDNPMCLKCDLQERLLCPSLLAGTADGSLRNDDPKGDF | | |
| | ITLYQMASQSSASHYKLQVIKALKSSGLCESLTYGLPFILRPTSCWQLDWDELETNQQ | | |
| | HFHALCHSLLKREWLLLAKGEPPGPGHSQRIPASTFYVIMPSHSLTLLVKAVATRELM | | |
| | LPSTFPLLPEDPHDDSLKNSMLDSLELEPTYNPLHVQSHLYSHLSSIYAKPQGRLHPH | | |
| | WESRAPRKTGQLQTNRARATVAPLPMTPVPGRASKNPAASKSSSDAFFLPSEWEKDPS | | |
| | RP | | |
| | SEQ ID NO:145 | 1647 bp | |
| NOV46b, | AAAGAATATCCAGCTGGTGGCTACAGTTCCCCCTCTGGTTTTGCTGCCATGCATCCTG | | |
| CG59383-02 DNA Sequence | | | |
| | GGCGAACTACTGGTAAAGGGCCCTCTACTCACACTCAGATTGACCAGCAACCTCCACG | | |
| | GCTTCTCATTGTGCACATTGCTCTACCGTCCTGGGCTGACATCTGCACCAACCTCTGT | | |
| | GAGGCTCTGCAGAACTTCTTCTCTCTAGCCTGCAGCTTGATGGGCCCCAGCCGCATGT | | |
| | CCCTGTTCAGTTTATACATGGTACAAGATCAGCATGAGTGCATCCTCCCTTTTGTGCA | | |
| | AGTGAAAOGGAACTTTGCTAGGTTGCAGACCTGCATCTCAGAACTCCGCATGTTACAG | | |
| | AGAGAAGGGTGTTTCAGATCACAAGGTGCTTCTCTGCGGCTGGCAGTAGAGGATGGGC | | |
| | TCCAGCAATTCAAACAATACAGCAGACATGTGACCACAAGGGCAGCTCTGACCTATAC | | |
| | CTCCCTGGAGATTACTATTCTGACTTCTCAGCCTGGAAAAGAGGTGGTCAAACAGTTG | | |
| | GAGGAAGGGTTGAAAGATACAGACCTAGCCAGAGTCAGGAGGTTTCAGGTCGTTGAGG | | |
| | TCACAAAGGGAATCCTAGAGCACGTGGACTCAGCGTCTCCTGTTGAGGATACCAGCAA | | |
| | TGATGAGAGTTCTATTCTGGGAACTGACATTGACCTTCAGACTATAGACAATGATATC | | |
| | GTCAGCATGGAGATTTTCTTCAAAGCCTGGCTACATAACAGTGGAACAGACCAGGAAC | | |
| | AAATCCATCTTCTTCTTTCTTCACAGTGTTTCAGCAACATTTCCAGACCCAGAGATAA | | |
| | TCCAATGTGTCTGAAATGTGATCTCCAAGAGCGACTGCTCTGCCCATCCCTACTCGCT | | |
| | GGCACAGCTGACGGCTCCTTGAGAATGGATGACCCTAAAGGAGACTTCATCACACTCC | | |
| | ACCAGATGGCTTCCCAGTCATCGGCCTCTCATTACAAGCTCCAAGTGATCAAGGCTTT | | |
| | AAAATCTAGCGGGCTCTGCGAGTCATTGACATATGGACTCCCGTTCATCCTCAGACCT | | |
| | ACAACCTGTTGGCAGCTGGACTGGGATGAGCTGGAGACAAATCAGCAACATTTCCATG | | |
| | CTTTGTGTCACAGCCTGCTGAAAAGGGAATGGCTGCTGTTAGCCAAGGGGGAACCACC | | |
| | GGGCCCAGGACACAGCCAGAGAATTCCTGCCAGCACCTTCTATGTGATCATGCCGTCA | | |
| | CACTCCCTCACACTGCTGGTAAAGGCGGTGGCCACGCGGCAACTGATGCTGCCCAGCA | | |
| | CCTTCCCCCTGCTGCCTGAGGACCCACATGATGATAGCCTTAAGAATGTGGAGAGCAT | | |
| | GCTGGACAGCCTGGAGCTGOAGCCCACCTACAACCCCTTGCATGTTCAAAGCCACCTG | | |
| | TACTCACACCTGAGCAGCATCTATGCCAAGCCTCAGGGGCGGCTCCACCCACACTGGG | | |
| | AGAGCCGAGCTCCGAGAAAGCATCCCTGCAAGACTGGGCAGTTGCAGACCAACCGAGC | | |
| | TCGAGCTACTGTGGCCCCCCTGCCTATGACTCCTGTCCCAGGCAGAGCCTCCAAGATG | | |
| | CCAGCAGCCAGCAAATCTTCCTCAGATGCCTTCTTCCTGCCTTCAGAGTGGGAGAAGG | | |
| | ATCCCTCAAGGCCCTAAGTCACC | | |
| | ORF Start: ATG at 49 | | ORF Stop: TAA at 1639 |
| | SEQ ID NO:146 | 530 aa | MW at 59359.1 kD |

| | |
|---|---|
| NOV46b, | MHPGRTTGKGPSTHTQIDQQPPRLLIVHIALPSWADICTNLCEALQNFFSLACSLMGP |
| CG59383-02 Protein Sequence | |
| | SRMSLFSLYMVQDQHECILPFVQVKGNFARLQTCISELRMLQREGCFRSQGASLRLAV |
| | EDGLQQFKQYSRHVTTRAALTYTSLEITILTSQPGKEVVKQLEEGLKDTDLARVRRFQ |
| | VVEVTKGTLEHVDSASPVEDTSNDESSILGTDIDLQTIDNDIVSMEIFFKAWLHNSGT |
| | DQEQIELLLSSQCFSNISRPRDNPMCLKCDLQERLLCPSLLAGTADGSLRMDDPKGDF |
| | ITLHQMASQSSASUYKLQVIKALKSSGLCESLTYGLPFILRPTSCWQLDWDELETNQQ |
| | HFHALCHSLLKREWLLLAKGEPPGPGHSQRIPASTFYVIMPSHSLTLLVKAVATRELM |
| | LPSTFPLLPEDPHDDSLKNVESMLDSLELEPTYNPLHVQSHLYSHLSSIYAKPQGRLH |
| | PHWESRAPRKHPCKTGQLQTNRARATVAPLPMTPVPGRASKMPAASKSSSDAPFLPSE |
| | WEKDPSRP |

Sequence comparison of the above protein sequences yields the following sequence relationships shown in Table 46B.

TABLE 46B
Comparison of NOV46a against NOV46b.

| | | Identities/ |
|---|---|---|
| Protein | NOV44a Residues/ | Similarities for |
| Sequence | Match Residues | the Matched Region |
| NOV46b | 1 . . . 524 | 509/530 (96%) |
| | 1 . . . 530 | 510/530 (96%) |

Further analysis of the NOV46a protein yielded the following properties shown in Table 46C.

TABLE 46C
Protein Sequence Properties NOV46a

| | |
|---|---|
| PSort | 0.4500 probability located in cytoplasm; 0.3000 |
| analysis: | probability located in microbody (peroxisome); |
| | 0.1000 probability located in mitochondrial matrix |
| | space; 0.1000 probability located in lysosome (lumen) |
| SignalP | No Known Signal Sequence Predicted |
| analysis: | |

A search of the NOV46a protein against the Geneseq database, a proprietary database that contains sequences published in patents and patent publication, yielded several homologous proteins shown in Table 46D.

TABLE 46D
Geneseq Results for NOV46a

| | | NOV46a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM34317 | Peptide #8354 encoded by probe for | 259 . . . 310 | 52/52 (100%) | 7e−23 |
| | measuring placental gene expression- | 1 . . . 52 | 52/52 (100%) | |
| | Homo sapiens, 52 aa. | | | |
| | [WO200157272-A2, 9 AUG. 2001] | | | |
| ABB18624 | Protein #623 encoded by probe for | 101 . . . 142 | 42/42 (100%) | 2e−16 |
| | measuring heart cell gene expression- | 1 . . . 42 | 42/42 (100%) | |
| | Homo sapiens, 42 aa. | | | |
| | [WO200157274-A2, 9 AUG. 2001] | | | |
| AAM66343 | Human bone marrow expressed | 101 . . . 142 | 42/42 (100%) | 2e−16 |
| | probe encoded protein SEQ ID NO: | 1 . . . 42 | 42/42 (100%) | |
| | 26649-Homo sapiens, 42 aa. | | | |
| | [WO200157276-A2, 9 AUG. 2001] | | | |
| AAM53955 | Human brain expressed single exon | 101 . . . 142 | 42/42 (100%) | 2e−16 |
| | probe encoded protein SEQ ID NO: | 1 . . . 42 | 42/42 (100%) | |
| | 26060-Homo sapiens, 42 aa. | | | |
| | [WO200157275-A2, 9 AUG. 2001] | | | |
| AAM26622 | Peptide #659 encoded by probe for | 101 . . . 142 | 42/42 (100%) | 2e−16 |
| | measuring placental gene expression- | 1 . . . 42 | 42/42 (100%) | |
| | Homo sapiens, 42 aa. | | | |
| | [WO200157272-A2, 9 AUG. 2001] | | | |

In a BLAST search of public sequence databases, the NOV46a protein was found to have homology to the proteins shown in the BLASTP data in Table 46E.

TABLE 46E
Public BLASTP Results for NOV46a

| | | NOV46a | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| Q9Z0E1 | D6MM5E PROTEIN - Mus | 1 . . . 524 | 380/526 | (72%) | 0.0 |
| | musculus (Mouse), 529 aa. | 1 . . . 526 | 423/526 | (80%) | |
| Q96L07 | SIMILAR TO DNA SEGMENT, | 1 . . . 358 | 358/358 | (100%) | 0.0 |
| | CHR 6, MIRIAM MEISLER 5, | 1 . . . 358 | 358/358 | (100%) | |
| | EXPRESSED - Homo sapiens | | | | |
| | (Human), 365 aa. | | | | |

PFam analysis predicts that the NOV46a protein contains the domains shown in the Table 46F.

TABLE 46F
Domain Analysis of NOV46a

| | | Identities/ | |
|---|---|---|---|
| | | Similarities | |
| | NOV46a | for the | |
| Pfam Domain | Match Region | Matched Region | Expect Value |
| RA: domain 1 of 1 | 124 . . . 214 | 18/115 (16%) | 8.4 |
| | | 65/115 (57%) | |

### Example 47

The NOV47 clone was analyzed, and the nucleotide and predicted polypeptide sequences are shown in Table 47A.

TABLE 47A
NOV47 Sequence Analysis

| | SEQ ID NO:147 | 960 bp | |
|---|---|---|---|
| NOV47a, | AGGACTAAATAAAATGGCCTAAATTTAAATATGGATTGGGATTTCCATTCTCTTGCAG | | |
| CG58526-01 DNA Sequence | | | |
| | ATGCCCAGAACCAAAGAAGAGGTCTGCCTGGTTTTCTTCCTGGAGCTCCAGACCCAGA | | |
| | CCAAAGCCTTCCTGCCTCTTCCAATCCAGGGAACCAAGCATGGCAGCTGAGTCTCCCT | | |
| | CTGCCAAGCAGTTTCCTGCCAACAGTCAGTCTCCCTCCTGGTCTAGAATATTTAAGCC | | |
| | AGTTAGACCTGATAATTATACACCAGCAGGTGGAGCTGCTTGTGATACTTGGTACTGA | | |
| | GACCTCCAACAAATATGAGATTAAAAACAGCTTGGGACAAAGAATTTACTTTGCAGTG | | |
| | GAGGAAAGCATCTGCTTCAATCGTACTTTCTGTTCCACTCTGCGATCTTGCACCCTGA | | |
| | GGATCACAGATAACTCAGGTCGAGAGGTCATTACAGTGAACAGGCCCTTGAGATGTAA | | |
| | CAGCTGCTGGTGCCCTTGCTACCTACAAGAGTTAGAAATCCAAGCCCCTCCTGGTACT | | |
| | ATAGTTGGTTACGTTACGCAGAAGTGGGACCCCTTTCTGCCTAAATTCACAATCCAAA | | |
| | ATGCAAACAAAGAAGATATTTTGAAAATTGTTGGTCCTTGTGTGACATGTGGCTGTTT | | |
| | TGGCGATGTGGATTTTGAGAAGGTGAAAACCATTAATGAAAAGCTTACAATTGGGAAG | | |
| | ATTTCAAAGTACTGGTCAGGATTTGTAAATGATGTCTTCACAAATGCTGACAATTTCG | | |
| | GAATTCATGTTCCTGCAGATCTAGATGTAACAGTCAAAGCAGCAATGATCGGTGCCTG | | |
| | TTTTCTCTTTGTAAGTATGGGCTTTGAGAGCCCAGCCCTCCAAGATGAGAAAGAGTCA | | |
| | GTGTGGCAATTCAAAAAATCAGAGTGCCCTCTCACCTCCAAACAAGCCCACTTGTTCC | | |
| | CCAGCGATGGTTCTTAGCCAGACTGAAATGAC | | |
| | ORF Start: ATG at 31 | | ORF Stop: TAG at 943 |
| | SEQ ID NO:148 | 304 aa | MW at 33794.2 kD |

| | |
|---|---|
| NOV47a, | MDWDFHSLADAQNQRRGLPGFLPGAPDPDQSLPASSNPGNQAWQLSLPLPSSFLPTVS |
| CG58526-01 Protein Sequence | |
| | LPPGLEYLSQLDLIIIHQQVELLVILGTETSNKYEIKNSLGQRIYFAVEESICFNRTF |
| | CSTLRSCTLRITDNSGREVITVNRPLRCNSCWCPCYLQELEIQAPPGTIVGYVTQKWD |
| | PFLPKFTIQNANKEDILRIVGPCVTCGCFGDVDFEKVKTINEKLTIGKISKYWSGFVN |
| | DVFTNADNFGIRVPADLDVTVKAAMIGACFLFVSMGFESPALQDEKESVWQFKKSECP |
| | LTSKQAHLFPSDGS |

Further analysis of the NOV47a protein yielded the following properties shown in Table 47B.

TABLE 47B
Protein Sequence Properties NOV47a

| | |
|---|---|
| PSort | 0.8500 probability located in endoplasmic reticulum |
| analysis: | (membrane); 0.4400 probability located in plasma |
| | membrane; 0.4244 probability located in microbody |
| | (peroxisome); 0.1000 probability located in |
| | mitochondrial inner membrane |
| SignalP | No Known Signal Sequence Predicted |
| analysis: | |

A search of the NOV47a protein against the Geneseq database, a proprietary database that contains sequences published in patents and patent publication, yielded several homologous proteins shown in Table 47C.

TABLE 47C
Geneseq Results for NOV47a

| | | NOV47a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG78341 | Human Mm-1 cell line derived | 24 . . . 282 | 152/263 (57%) | 5e−84 |
| | transplantability-associated gene 1b - | 60 . . . 318 | 187/263 (70%) | |
| | Homo sapiens , 318 aa. | | | |
| | [WO200164894-A2, 7 SEP 2001] | | | |
| AAB24113 | Human phospholipid scramblase | 24 . . . 282 | 152/263 (57%) | 5e−84 |
| | HPLS protein sequence - Homo | 60 . . . 318 | 187/263 (70%) | |
| | sapiens , 318 aa. [CN1259574-A, | | | |
| | 12 JUL 2000] | | | |
| AAB24112 | Mouse phospholipid scramblase | 24 . . . 282 | 152/263 (57%) | 5e−84 |
| | MPLS protein sequence - Mus sp, | 60 . . . 318 | 187/263 (70%) | |
| | 318 aa. [CN1259574-A, 12 JUL | | | |
| | 2000] | | | |
| AAY09309 | Human phospholipid scramblase - | 24 . . . 282 | 152/263 (57%) | 5e−84 |
| | Homo sapiens , 318 aa. | 60 . . . 318 | 187/263 (70%) | |
| | [WO9919352-A2, 22 APR 1999] | | | |
| AAY29323 | Human PL scramblase - Homo | 24 . . . 282 | 152/263 (57%) | 5e−84 |
| | sapiens , 318 aa. [WO9936536-A2, | 60 . . . 318 | 187/263 (70%) | |
| | 22 JUL 1999] | | | |

In a BLAST search of public sequence databases, the NOV47a protein was found to have homology to the proteins shown in the BLASTP data in Table 47D.

TABLE 47D
Public BLASTP Results for NOV47a

| | | | | |
|---|---|---|---|---|
| | | NOV47a | Identities/ | |
| Protein | | Residues/ | Similarities for | |
| Accession | | Match | the Matched | Expect |
| Number | Protein/Organism/Length | Residues | Portion | Value |

| | | | | |
|---|---|---|---|---|
| Q9JJ00 | Phospholipid scramblase 1 (PL | 20 . . . 283 | 150/267 (56%) | 4e−84 |
| | scramblase 1) (Transplantability | 66 . . . 328 | 191/267 (71%) | |
| | associated protein 1) (TRA1) (NOR1) - | | | |
| | Mus musculus (Mouse), 328 aa. | | | |
| Q99M50 | PHOSPHOLIPID SCRAMBLASE 1 - | 20 . . . 282 | 150/266 (56%) | 6e−84 |
| | Mus musculus (Mouse), 327 aa. | 66 . . . 327 | 191/266 (71%) | |
| O15162 | Phospholipid scramblase 1 (PL | 24 . . . 282 | 152/263 (57%) | 2e−83 |
| | scramblase 1) (Erythrocyte | 60 . . . 318 | 187/263 (70%) | |
| | phospholipid scramblase) (Ca2 + | | | |
| | dependent phospholipid scramblase 1) | | | |
| | (MmTRA1b) - Homo sapiens | | | |
| | (Human), 318 aa. | | | |
| P58195 | Phospholipid scramblase 1 (PL | 28 . . . 282 | 145/256 (56%) | 3e−81 |
| | scramblase 1) (Ca2 + dependent | 84 . . . 335 | 183/256 (70%) | |
| | phospholipid scramblase 1) - Rattus | | | |
| | norvegicus (Rat), 335 aa. | | | |
| Q9NRY7 | Phospholipid scramblase 2 (PL | 55 . . . 270 | 135/217 (62%) | le−75 |
| | scramblase 2) (Ca2 + dependent | 6 . . . 221 | 164/217 (75%) | |
| | phospholipid scramblase 2) - Homo | | | |
| | sapiens (Human), 224 aa. | | | |

PFam analysis predicts that the NOV47a protein contains the domains shown in the Table 47E.

TABLE 47E
Domain Analysis of NOV47a

| | | | |
|---|---|---|---|
| | | Identities/ | |
| Pfam | NOV47a | Similarities | Expect |
| Domain | Match Region | for the Matched Region | Value |

No Significant Matches Found

### Example 48

The NOV48 clone was analyzed, and the nucleotide and predicted polypeptide sequences are shown in Table 48A.

TABLE 48A
NOV48 Sequence Analysis

| | SEQ ID NO:149 | 957 bp | |
|---|---|---|---|
| NOV48a, | CCCCTGCTGGTGCCCAAGACCACCGTGGAAGGAATGGCTAAAGAGGAGACAAGTGAGT | | |
| CG57851-01 DNA Sequence | | | |
| | TAGAATGGGGCTTGTTACCCCCAGAAGAATTTTCCCAAGTGAATGGAATCATTCTTCA | | |
| | AAAGAAAATGTGCGATTTCTGGGATAAGATCTGGAACTTCCAAGCCAAGCCTGATGAC | | |
| | CTGCTCATTGCTTCTTACCCCAAAGCAGGTACCACTTGGACACAGGAAATTGTAGATC | | |
| | TGATACAAAATGATGGCGATATTGAGAAAAGCAGGCGCGCTTCCATTCAACTTCAACA | | |
| | CCCTTTCCTGGAGTGGATAAGAATGACACACGCCAGGAAAATTTTTGCAGGGATTGAC | | |
| | CAGGCTAACACAATGCCTTCCCCAAGGACCCTGAAAACTCATCTTCCTGTACAACTAC | | |
| | TGCCTCCATCCTTCTGGGAGGAAAACTGTAAGATAATCTATGTGGCAAGAAATGCCAA | | |
| | GGATAACCTGGTGTCCTACTACCATTTTCAAAGGATGAGCAAAGCACTCCCTGACGTT | | |
| | TTGACAGTGGGAGAATACATTATGTGTGGGGAAGTGTTGTGGGGAATATGGGAAGAGA | | |
| | TTCGGACTTGGCAACTGCATAGGTTGTTCTGCTGGTTCTTTGATCATGCTTCTGAGAA | | |
| | TCCTAGAAAGTTCAAAAGGATAATGGAATTTATGGGGAATAAACTAGATGAAGATCCT | | |
| | GTCAAAAGAATTGTTCAGCACACATCTTTTGAAAGTAAGAAGAAAAACCAGATGACCA | | |
| | ACTATGTAATGATAACCTGTGACATCATGGACCACTCCATCTCCCCATTTATGAGGAA | | |
| | AGGGACCGTTGGAGAGTGGAAGGATTACTTCTCAGCAGCACACAATAAGAGATTTGAT | | |
| | GAAGACAGGAAAATGGCTGACTCTTCTCTGACCTTCCACACGGAGCTCTAAAGAGAGA | | |
| | GAGACAAAGTCTATACTACACAGGGGCAC | | |
| | ORF Start: ATG at 34 | | ORF Stop: TAA at 919 |
| | SEQ ID NO:150 | 295 aa | MW at 34853.7 kD |

| | |
|---|---|
| NOV48a, | MAKEETSELEWGLLPPEEFSQVNGIILQKKMCDFWDKIWNFQAKPDDLLIASYPKAGT |
| CG57851-01 Protein Sequence | |
| | TWTQEIVDLIQNDGDIEKSRRASIQLQHPFLEWIRMTHARKIFAGIDQANTMPSPRTL |
| | KTHLPVQLLPPSFWEENCKIIYVARNAKDNLVSYYHFQRMSKALPDVLTVGEYIMCGE |
| | VLWGIWEEIRTWQLHRLFCWFFDHASENPRKFKRIMEFMGNKLDEDPVKRIVQHTSFE |
| | SKKKNQMTNYVNITCDIMDESISPFMRKGTVGEWKDYFSAAQNKRFDEDRKMADSSLT |
| | FHTEL |

Further analysis of the NOV48a protein yielded the following properties shown in Table 48B.

TABLE 48B
Protein Sequence Properties NOV48a

| | |
|---|---|
| PSort | 0.6400 probability located in microbody (peroxisome); |
| analysis: | 0.4500 probability located in cytoplasm; 0.1000 probability |
| | located in mitochondrial matrix space; 0.1000 probability |
| | located in lysosome (lumen) |
| SignalP | No Known Signal Sequence Predicted |
| analysis: | |

A search of the NOV48a protein against the Geneseq database, a proprietary database that contains sequences published in patents and patent publication, yielded several homologous proteins shown in Table 48C.

TABLE 48C
Geneseq Results for NOV48a

| | | NOV48a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAE12209 | Human ST drug-metabolising | 16 . . . 295 | 137/293 (46%) | 9e−74 |
| | protein 2 encoded by DNA | 15 . . . 304 | 200/293 (67%) | |
| | transcript 2 - Homo sapiens, 304 aa. | | | |
| | [WO200172977-A2, 4 OCT 2001] | | | |
| AAE12210 | Human ST drug-metabolising | 16 . . . 295 | 129/293 (44%) | 1e−67 |
| | protein 3 encoded by cDNA - Homo | 15 . . . 304 | 190/293 (64%) | |
| | sapiens, 304 aa. [WO200172977- | | | |
| | A2, 4 OCT 2001] | | | |
| AAE12208 | Human ST drug-metabolising | 16 . . . 295 | 128/293 (43%) | 6e−67 |
| | protein 1 encoded by DNA | 15 . . . 304 | 190/293 (64%) | |
| | transcript 1 - Homo sapiens, 304 aa. | | | |
| | [WO200172977-A2, 4 OCT 2001] | | | |
| AAE05178 | Human drug metabolising enzyme | 16 . . . 295 | 128/293 (43%) | 1e−66 |
| | (DME-9) protein - Homo sapiens, | 15 . . . 304 | 189/293 (63%) | |
| | 304 aa. [WO200151638-A2, | | | |
| | 19 JUL 2001] | | | |
| AAY67294 | Human STP2 (phenol | 15 . . . 295 | 133/292 (45%) | 5e−66 |
| | sulphotransferase 2) amino acid | 10 . . . 295 | 186/292 (63%) | |
| | sequence - Homo sapiens, 295 aa. | | | |
| | [WO9964630-A1, 16 DEC 1999] | | | |

In a BLAST search of public sequence databases, the NOV48a protein was found to have homology to the proteins shown in the BLASTP data in Table 48D.

TABLE 48D
Public BLASTP Results for NOV48a

| | | NOV48a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q90WR6 | SULFOTRANSFERASE 1C - | 3 . . . 295 | 170/304 (55%) | 3e−94 |
| | Gallus gallus (Chicken), 307 aa. | 5 . . . 307 | 218/304 (70%) | |
| P50237 | N-hydroxyarylamine | 1 . . . 295 | 172/308 (55%) | 3e−92 |
| | sulfotransferase (EC 2.8.2.-) | 1 . . . 304 | 222/308 (71%) | |
| | (HAST-I) - Rattus norvegicus | | | |
| | (Rat), 304 aa. | | | |
| O70262 | PHENOL SULFOTRANSFERASE - | 18 . . . 295 | 164/289 (56%) | 1e−91 |
| | Mus musculus (Mouse), 304 aa. | 19 . . . 304 | 215/289 (73%) | |
| O75897 | Sulfotransferase 1C2 (EC 2.8.2.-) | 22 . . . 292 | 160/282 (56%) | 1e−87 |
| | (SULT1C) (SULT1C#2) - Homo | 22 . . . 299 | 203/282 (71%) | |
| | sapiens (Human), 302 aa. | | | |
| O00338 | Sulfotransferase 1C1 (EC 2.8.2.-) | 18 . . . 295 | 149/289 (51%) | 1e−80 |
| | (SULT1C#1) (ST1C2) | 12 . . . 296 | 201/289 (68%) | |
| | (humSULTC2) - Homo sapiens | | | |
| | (Human), 296 aa. | | | |

PFam analysis predicts that the NOV48a protein contains the domains shown in the Table 48E.

TABLE 48E
Domain Analysis of NOV48a

| | | Identities/ | |
|---|---|---|---|
| Pfam | NOV48a | Similarities | Expect |
| Domain | Match Region | for the Matched Region | Value |
| Sulfotransfer: | 23 . . . 285 | 116/298 (39%) | 6.2e−82 |
| domain 1 of 1 | | 207/298 (69%) | |

### Example 49

The NOV49 clone was analyzed, and the nucleotide and predicted polypeptide sequences are shown in Table 49A.

TABLE 49A
NOV49 Sequence Analysis

| | SEQ ID NO:151 | 1934 bp | |
|---|---|---|---|
| NOV49a, | CTTGATTACGGAGACTGAACCTTCATAGGGTGCGCACTTACCAAGGACAGGAAGGTTT | | |
| CG59377-01 DNA Sequence | | | |
| | CTCTGTTTGAAGGGCTTTAAACTTATAACAAAGAAAATAAAAATGACGACTTCGTCTA | | |
| | TCAGACGGCAGATGAAAAACATCGTGAACAATTACTCAGAGGCAGAAATCAAAGTCCG | | |
| | GGAAGCCACCTCCAATGACCCGTGGGGCCCGTCCAGTTCTCTGATGACCGAGATTGCC | | |
| | GACCTGACCTACAACGTGGTGGCCTTCTCGGAGATCATGAGCATGGTGTGGAAGCGGC | | |
| | TGAATGACCATGGCAAGAACTGGCGGCATGTGTACAAGGCGCTGACCCTGCTGGACTA | | |
| | CCTCATCAAGACAGGCTCCGAACGTGTGGCCCAGCAGTGCCGGGAGAACATCTTCGCC | | |
| | ATCCAGACCCTGAAGGACTTCCAGTACATTGACCOAGATGGCAAGGACCAGGGCATCA | | |
| | ATGTGCGTGAGAAGTCAAAGCAACTGGTGGCTCTCCTCAAGGACGAGGAACGGTTGAA | | |
| | GGCTGAGAGGGCCCAGGCTCTCAAAACCAAAGAGCGCATGGCCCAGGTTGCCACTGGC | | |
| | ATGGGCAGCAACCAGATCACCTTTGGGCGAGGCTCCAGCCAGCCCAACCTCtCCACCA | | |
| | GCCACTCGGAGCAGGAGTATGGCAAGGCCGGGGGCTCCCCGGCCTCCTACCATGGCTC | | |
| | CACCTCCCCGCGAGTGTCCTCCGAGCTGGAGCAAGCCCGGCCCCAGACTAGTGGAGAA | | |
| | GAGGAGCTTCAGCTGCAGCTGGCACTTGCCATGAGCAGAGAAGTGGCTGAGCAGGAAG | | |
| | AACGCCTCAGGCGGGGTGATGACCTCAGATTACAGATGGCCCTGGAAGAAAGCCGAAG | | |
| | GGACACAGTTAAAATTCCAAAAAAGAAAGAGCAGACTACGCTGTTGGATTTAATGGAT | | |
| | GCTCTCCCCAGCTCGGGCCCCGCGGCCCACAAAGCAGAGCCCTGGGGCCCGTCAGCCT | | |
| | CCACTAACCAGACCAACCCCTGGGGCGGGCCAGCGGCTCCTGCGAGTACTTCAGACCC | | |
| | CTGGCCATCGTTTGGTACCAAGCCAGCTGCCTCCATTGACCCATGGGGGGTGCCCACT | | |
| | GGAGCCACCGCACAATCTGTCCCCAAGAACTCGGACCCCTGGGCAGCTTCACAGCAGC | | |
| | CTGCCTCCAGTGCTGGGAAAAGAGCTTCTGACGCGTGGGGCGCAGTCTCCACCACCAA | | |
| | GCCCGTGTCTGTCTCTGGGTCCTTTGAGCTCTTCAGTAATCTGAATGGTACAATTAAA | | |
| | GATGACTTTTCTGAATTTGACAACCTTCGGACTTCAAAAAAAACAGCCGAATCTGTGA | | |
| | CCTCTCTGCCATCCCAAAACAATGGAACTACCAGCCCTGACCCCTTTGAGTCTCAACC | | |
| | CCTGACTGTCGCCTCAAGCAAGCCCAGCAGTGCCCGGAAAACACCTGAGTCCTTCCTG | | |
| | GGCCCCAACGCGGCCCTGGTGAACCTGGACTCACCGGTGACCAGGCCTGCCCCACCAG | | |
| | CCCAGTCCCTCAACCCTTTCCTGGCACCAGGTGCTCCCGCCACCTCGGCCCCTGTTAA | | |
| | CCCTTTCCAGGTGAACCAGCCCCAGCCGCTGACACTGAACCAGCTTCGGGGGAGCCCA | | |
| | GTCCTGGGGACCAGCACATCCTTTGGGCCTGGCCCAGGAGTGGAGTCCATGGCTGTGG | | |
| | CCTCGATGACCTCCGCGGCCCCACAGCCAGCTCTGGGGGCCACTGGTTCCTCTCTGAC | | |
| | ACCACTGGGCCCTGCAATGATGAACATGGTGGGCAGTGTCGGTATACCCCCATCAGCA | | |
| | GCCCAGGCCACTGGCACAACCAACCCTTTCCTTCTCTAGTGCCTGGGCCTGGGACCCA | | |
| | CCCAGAGCACCTGTGCTGGAGGATGCCGAGCAGGCACTCTCGTCTGTGGCACGGGATC | | |
| | CAAGAGTTTGGQGATTAGGG | | |
| | ORF Start: ATG at 101 | | ORF Stop: TAG at 1835 |
| | SEQ ID NO:152 | 578 aa | MW at 61651.2 kD |

| | |
|---|---|
| NOV49a, | MTTSSIRRQNKNIVNNYSEAEIKVREATSNDPWGPSSSLMTEIADLTYNVVAFSEIMS |
| CG59377-01 Protein Sequence | |
| | MVWKRLNDHGKNWRHVYKALTLLDYLIKTGSERVAQQCRENIFAIQTLKDFQYIDRDG |
| | KDQGINVREKSKQLVALLKDEERLKAERAQALKTKERMAQVATGMGSNQITFGRGSSQ |
| | PNLSTSESEQEYGKAGGSPASYHGSTSPRVSSELEQARPQTSGEEELQLQLALAISRE |
| | VAEQEERLRRGDDLRLQMALEESRRDTVKIPKKKEQTTLLDLMDALPSSGPAAQKAEP |
| | WGPSASTNQTNPWGGPAAPASTSDPWPSFGTKPAASIDPWGVPTGATAQSVPKNSDPW |
| | AASQQPASSAGKRASDAWGAVSTTKPVSVSGSFELFSNLNGTIKDDFSEFDNLRTSKK |
| | TAESVTSLPSQNNGTTSPDPFESQPLTVASSKPSSARKTPESFLGPNAALVNLDSLVT |
| | RPAPPAQSLNPFLAPGAFATSAPVNPFQVNQPQPLTLNQLRGSPVLGTSTSFGPGPGV |
| | ESMAVASMTSAAPQPATGATGSSLTPLGPAMMNMVGSVGIPPSAAQATGTTNPFLL |

Further analysis of the NOV49a protein yielded the following properties shown in Table 49B.

TABLE 49B
Protein Sequence Properties NOV49a

| | |
|---|---|
| PSort | 0.4936 probability located in mitochondrial matrix space; |
| analysis: | 0.3000 probability located in nucleus; 0.2087 probability |
| | located in mitochondrial inner membrane; 0.2087 probability |
| | located in mitochondrial intermembrane space |
| SignalP | No Known Signal Sequence Predicted |
| analysis: | |

A search of the NOV49a protein against the Geneseq database, a proprietary database that contains sequences published in patents and patent publication, yielded several homologousproteins shown in Table 49C.

TABLE 49C
Geneseq Results for NOV49a

| | | NOV49a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues/ | Region | Value |
| AAB93525 | Human protein sequence SEQ ID | 1 . . . 578 | 578/584 (98%) | 0.0 |
| | NO:12872 - Homo sapiens , 584 aa. | 1 . . . 584 | 578/584 (98%) | |
| | [EP1074617-A2, 7 FEB 2001] | | | |
| AAB95663 | Human protein sequence SEQ ID | 40 . . . 403 | 364/370 (98%) | 0.0 |
| | NO: 18438 - Homo sapiens, 370 aa. | 1 . . . 370 | 364/370 (98%) | |
| | [EP1074617-A2, 7 FEB 2001] | | | |
| AAB93011 | Human protein sequence SEQ ID | 1 . . . 407 | 385/470 (81%) | 0.0 |
| | NO:11762 - Homo sapiens , 484 aa. | 1 . . . 470 | 390/470 (82%) | |
| | [EP1074617-A2, 7 FEB 2001] | | | |
| AAB42049 | Human ORFX ORF1813 | 1 . . . 578 | 306/636 (48%) | e−141 |
| | polypeptide sequence SEQ ID | 1 . . . 551 | 370/636 (58%) | |
| | NO:3626 - Homo sapiens, 551 aa. | | | |
| | [WO200058473-A2, 5 OCT 2000] | | | |
| AAB95100 | Human protein sequence SEQ ID | 1 . . . 578 | 298/636 (46%) | e−137 |
| | NO:17064 - Homo sapiens, 576 aa. | 1 . . . 576 | 371/636 (57%) | |
| | [EP1074617-A2, 7 FEB 2001] | | | |

In a BLAST search of public sequence databases, the NOV49a protein was found to have homology to the proteins shown in the BLASTP data in Table 49D.

TABLE 49D
Public BLASTP Results for NOV49a

| | | | Identities/ | |
|---|---|---|---|---|
| Protein | | NOV49a | Similarities for | |
| Accession | | Residues/Match | the Matched | Expect |
| Number | Protein/Organism/Length | Residues | Portion | Value |
| O95207 | EPSIN 2A - Homo sapiens | 1 . . . 578 | 576/584 (98%) | 0.0 |
| | (Human), 584 aa. | 1 . . . 584 | 576/584 (98%) | |
| Q9UPT7 | KIAA1065 PROTEIN - Homo | 1 . . . 578 | 557/641 (86%) | 0.0 |
| | sapiens (Human), 641 aa. | 1 . . . 641 | 562/641 (86%) | |
| O95208 | EPSIN 2B - Homo sapiens | 1 . . . 578 | 556/642 (86%) | 0.0 |
| | (Human), 642 aa. | 1 . . . 642 | 560/642 (86%) | |
| Q9Z1Z3 | EH DOMAIN BINDING | 1 . . . 578 | 512/590 (86%) | 0.0 |
| | PROTEIN EPSIN 2 - Rattus | 1 . . . 583 | 526/590 (88%) | |
| | norvegicus (Rat), 583 aa. | | | |
| O70447 | INTERSECTIN-EH BINDING | 76 . . . 578 | 438/515 (85%) | 0.0 |
| | PROTEIN IBP2 - Mus musculus | 2 . . . 509 | 459/515 (89%) | |
| | (Mouse), 509 aa (fragment). | | | |

PFam analysis predicts that the NOV49a protein contains the domains shown in the Table 49E.

TABLE 49E
Domain Analysis of NOV49a

| | | Identities/ | | |
|---|---|---|---|---|
| Pfam | NOV49a | Similarities | | Expect |
| Domain | Match Region | for the Matched Region | | Value |
| ENTH: domain | 17 . . . 140 | 70/131 | (53%) | 7.9e−68 |
| 1 of 1 | | 117/131 | (89%) | |
| VHS: domain 1 of 1 | 14 . . . 158 | 33/160 | (21%) | 3.3 |
| | | 90/160 | (56%) | |
| UIM: domain 1 of 2 | 217 . . . 234 | 11/18 | (61%) | 0.043 |
| | | 16/18 | (89%) | |
| UIM: domain 2 of 2 | 242 . . . 259 | 5/18 | (28%) | 80 |
| | | 12/18 | (67%) | |

### Example 50

The NOV50 clone was analyzed, and the nucleotide and predicted polypeptide sequences are shown in Table 50A.

TABLE 50A
NOV50 Sequence Analysis

| | |
|---|---|
| SEQ ID NO:153 | 2580 bp |

| NOV50a, | ATGCTGCTGGCCCCCTTTTATTGCTGGGTGTGTGCCCATGCTGCTGGCCCCCTTTTAT | | |
|---|---|---|---|
| CG59258-01 DNA Sequence | | | |
| | TGCTGGGCAGTGACAAACTGTACCATCAGTGGCTCTCCACTGTCCGGAAAGGAAGTGG | | |
| | AGCAATTCTGAATACTGTAAAGACCAAAGCAAATCCGGCCATGAAGACTGTCTACAAG | | |
| | TTCGACATTGCCGAGAATGGCTGCGCCCCCACCCCAGAAGAGCAGCTGCCAAAGACTG | | |
| | CACCGTCCCCACTGGTGGAGGCCAAGGACCCCAAGCTCCGAGAAGACCGGCGGCCAAT | | |
| | CACAGTCCACTTTGGACAGGACCAGTCTGAGATGTCTTTCAGCTCAGCACTCACTCAC | | |
| | GGCAAAGAGAGTGCCCGGACCCAGCCGGAGAGAGTCGTTGACAGGACTGGCGAGCCCC | | |
| | TGAATCCTGAGCGCGCTCTCTCCGGAGATCATCTCTGGCCTGTTACGCACTTGCTCTG | | |
| | GGCAACCCTGGGCAAGTCCTTGCTTGCCCTCATCTGTGAAATGGGTAGCAGCCCTCGT | | |
| | TCCCTGCAGAGGAGCCTTGCGCTGCTGGGGACACCCCAGCTTATTTGGGAAACTGCAA | | |
| | CCACCATGGCCGATGGCCCCACCACGCCCTGTCTAGGAAGCAGAGGCCTCCCCAGCAG | | |
| | CGTGTCCACTGTGCCCCTGGCCCTGCGTGAAGTGCCATCAGATGCCCCGCATCCCTGC | | |
| | AGCAGGGCCCTCGTGACTGGCGTCACAGATGAGGACACAGAGGCCCAGGGAAGTCACT | | |
| | TGCTTGCCAAAGTCACTCAGCAAACCATGTCTGTCTGGCTCTCAGAAAATGGGAAAGA | | |
| | AGCCTGGGCATTCAGCCATGAGGGAGCCACGGCTGTAGCCAGTGGAATGACGTACCCT | | |
| | CAGTCCAGGATGTGCACCCGGGCAGCCAGGTCCCACAGCCACTACTTTCTTGCCCCCA | | |
| | CCACTGCTCCCACAGTTCCCAGAACTCAGTCTCCAGATCTGGGCTCCAGGATGCAGAG | | |
| | GCTGTCCTCAGGCCTGGTAAAGCCCTTGCGACACTATGCGGTCTTCCTCTCCGAAGAC | | |
| | TCCTCTGATGATGAATGCCAGCGGGAAGAGGGCCCGAGCTCTGGCTTCACCGAGAGCT | | |
| | TTTTCTTCTCCGCTCCCTTTGAATGGCCGCAGCCGTATCGGACACTCAGGGAGTCAGA | | |
| | CAGCGCGGAAGGCGACGAGGCAGAGAGTCCAGAGCAGCAAGTGCGGAAGTCCACAGGC | | |
| | CCTGTCCCAGCTCCCCCTGACCGGGCTGCCAGCATCGACCTTCTGGAAGACGTCTTCA | | |
| | GCAACCTGGACATGGAGGCCGCACTGCAGCCACTGGGCCAGGCCAAGAGCTTAGAGGA | | |
| | CCTTCGTGCCCCCAAAGACCTGAGGGAGCAGCCAGGGACCTTTGACTATCAGAGGCTG | | |
| | GATCTGGGCGGGAGTGAGAGGAGCCGCGGGGTGACAGTGGCCTTGAAGCTTACCCACC | | |
| | CGTACAACAAGCTCTGGAGCCTGGGCCAGGACGACATGGCCATCCCCAGCAAGCCCCC | | |
| | AGCTGCCTCCCCTGAGAAGCCCTCAGCCCTGCTCGGAAACTCCCTGGCCCTGCCTCGA | | |
| | AGGCCCCAGAACCGGGACAGCATCCTGAACCCCAGTGACAAGGAGGAGGTGCCCACCC | | |
| | CTACTCTGGGCAGCATCACCATCCCCCGGCCCCAAGGCAGGAAGACCCCAGAGCTGGG | | |
| | CATCGTGCCTCCACCGCCCATTCCCCGCCCGGCCAAGCTCCAGGCTGCCGGCGCCGCA | | |
| | CTTGGTGACGTCTCAGAGCGGCTGCAGACGGATCGGGACAGGCGAGCTGCCCTGAGTC | | |
| | CAGGGCTCCTGCCTGGTGTTGTCCCCCAAGGCCCCACTGAACTGCTCCAGCCGCTCAG | | |
| | CCCTGGCCCCGGGGCTGCAGGCACGAGCAGTGACGCCCTGCTCGCCCTCCTGGACCCG | | |
| | CTCAGCACAGCCTGGTCAGGCAGCACCCTCCCGTCACGCCCCGCCACCCCGAATGTAG | | |
| | CCACCCCATTCACCCCCCAATTCAGCTTCCCCCCTGCAGGGACACCCACCCCATTCCC | | |
| | ACAGCCACCACTCAACCCCTTTGTCCCATCCATGCCAGCAGCCCCACCCACCCTGCCC | | |
| | CTGGTCTCCACACCAGCCGGGCCTTTCGGGGCCCCTCCAGCTTCCCTGGGGCCGGCTT | | |
| | TTGCGTCCGGCCTCCTGCTGTCCAGTGCTGGCTTCTGTGCCCCTCACAGGTCTCAGCC | | |
| | CAACCTCTCCGCCCTCTCCATGCCCAACCTCTTTGGCCAGATGCCCATGGGCACCCAC | | |
| | ACGAGCCCCCTACAGCCGCTGGGTCCCCCAGCAGTTGCCCCGTCGAGGATCCGAACGT | | |
| | TGCCCCTGGCCCGCTCAAGTGCCAGGGCTGCTGAGACCAAGCAGGGGCTCGCCCTGAG | | |
| | GCCTGGAGACCCCCCGCTTCTGCCTCCCAGGCCCCCTCAAGGCCTGGAGCCAACACTG | | |
| | CAGCCCTCTGCTCCTCAACAGGCCAGAGACCCCTTTGAGGATTTGTTACAGAAAACCA | | |
| | AGCAAGACGTGAGCCCGAGTCCGGCCCTGGCCCCGGCCCCAGACTCGGTGGAGCAGCT | | |
| | CAGGAAGCAGTGGGAGACCTTCGAGTGA | | |
| | ORF Start: ATG at 1 | | ORF Stop: TGA at 2578 |
| | SEQ ID NO:154 | 859 aa | MW at 91746.7 kD |

| | |
|---|---|
| NOV50a, | MLLAPFYCWVCAHAAGPLLLLGSDKLYHQWLSTVRKGSGAILNTVKTKANPAMKTVYK |
| CG59258-01 Protein Sequence | |
| | FDIAENGCAPTPEEQLPKTAPSPLVEAKDPKLREDRRPITVHFCQDQSEMSFSSALTH |
| | GKESARTQPERVVDRTGEPLNPERALSGDHLWPVTELLWATLGKSLLALICEMGSSPR |
| | SLQRSLALLGTPQLIWETATTMADGPTTPCLGSRGLPSSVSTVPLALREVPSDAPHPC |
| | SRALVTGLTDEDTEAQGSHLLAXVTQQTMSVWLSENGKEAWAFSHEGATAVASGMTYP |
| | QSRMCTRAARSHSHYFLAPTTAPTVPRTQSPDLGSRMQRLSSGLVKPLRHYAVFLSED |
| | SSDDECQREEGPSSGFTESFFFSAPFEWPQPYRTLRESDSAEGDEAESPEQQVRKSTG |
| | PVPAPPDRAASIDLLEDVFSNLDMEAALQPLGQAKSLEDLRAPKDLREQPGTFDYQRL |
| | DLGGSERSRGVTVALKLTHPYNKLWSLGQDDMAIPSKPPAASPEKPSALLGNSLALPR |
| | RPQNRNSILNPSDKEEVPTPTLGSITIPRPQGRKTPELGIVPPPPIPRPAKLQAAGAA |
| | LGDVSERLQTDRDRRAALSPGLLPGVVPQGPTELLQPLSPGPGAAGTSSDALLALLDP |
| | LSTAWSGSTLPSRPATPNVATPPTPQFSFPPAGTPTPFPQPPLNPFVPSMPAAPPTLP |
| | LVSTPAGPFGAPPASLGPAFASGLLLSSAGFCAPHRSQPNLSALSMPNLFGQMPNGTH |
| | TSPLQPLGPPAVAPSRIRTLPLARSSARAAETKQGLALRPGDPPLLPPRPPQGLEPTL |
| | QPSAPQQARDPFEDLLQKTKQDVSPSPALAPAPDSVEQLRKQWETFE |

Further analysis of the NOV50a protein yielded the following properties shown in Table 50B.

TABLE 50B
Protein Sequence Properties NOV50a

| | |
|---|---|
| PSort | 0.4500 probability located in cytoplasm; 0.3000 |
| analysis: | probability located in microbody (peroxisome); |
| | 0.1940 probability located in lysosome (lumen); |
| | 0.1000 probability located in mitochondrial matrix space |
| SignalP | Likely cleavage site between residues 15 and 16 |
| analysis: | |

A search of the NOV50a protein against the Geneseq database, a proprietary database that contains sequences published in patents and patent publication, yielded several homologous proteins shown in Table 50C.

TABLE 50C
Geneseq Results for NOV50a

| | | NOV50a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM41501 | Human polypeptide SEQ ID NO | 22 . . . 103 | 82/82 (100%) | 2e−42 |
| | 6432-Homo sapiens, 545 aa. | 401 . . . 482 | 82/82 (100%) | |
| | [WO200153312-A1, | | | |
| | 26 JUL. 2001] | | | |
| AAM39715 | Human polypeptide SEQ ID NO | 22 . . . 103 | 82/101 (81%) | 6e−39 |
| | 2860-Homo sapiens, 559 aa. | 396 . . . 496 | 82/101 (81%) | |
| | [WO200153312-A1, | | | |
| | 26 JUL. 2001] | | | |
| AAW31855 | Mycobacterium tuberculosis 55 | 498 . . . 845 | 96/358 (26%) | 8e−12 |
| | kDa protein-Mycobacterium | 71 . . . 389 | 125/358 (34%) | |
| | tuberculosis, 572 aa. | | | |
| | [WO9741252-A2, | | | |
| | 6 NOV. 1997] | | | |
| AAW31852 | Mycobacterium tuberculosis 74 | 498 . . . 845 | 96/358 (26%) | 8e−12 |
| | kDa protein-Mycobacterium | 262 . . . 580 | 125/358 (34%) | |
| | tuberculosis, 763 aa. | | | |
| | [WO9741252-A2, | | | |
| | 6 NOV. 1997] | | | |
| AAB50363 | Human SRCAP-Homo sapiens, | 501 . . . 845 | 112/369 (30%) | 1e−11 |
| | 2972 aa. [WO200073467-A1, | 1235 . . . 1575 | 141/369 (37%) | |
| | 7 DEC. 2000] | | | |

In a BLAST search of public sequence databases, the NOV50a protein was found to have homology to the proteins shown in the BLASTP data in Table 50D.

TABLE 59D
Public BLASTP Results for NOV50a

| | | NOV50a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9HCG4 | KIAA1608 PROTEIN-Homo | 309 . . . 859 | 501/555 (90%) | 0.0 |
| | sapiens (Human), 603 aa | 62 . . . 603 | 510/555 (91%) | |
| | (fragment). | | | |
| Q9H796 | CDNA: FLJ21129 FIS, CLONE | 22 . . . 103 | 81/101 (80%) | 2e−37 |
| | CAS06266-Homo sapiens | 396 . . . 496 | 81/101 (80%) | |
| | (Human), 559 aa. | | | |
| AAK44515 | HYPOTHETICAL 58.5 KDA | 499 . . . 845 | 104/354 (29%) | 8e−14 |
| | PROTEIN-Mycobacterium | 299 . . . 562 | 121/354 (33%) | |
| | tuberculosis CDC1551, 598 aa. | | | |
| Q9SN46 | EXTENSIN-LIKE PROTEIN- | 604 . . . 848 | 73/249 (29%) | 3e−12 |
| | Arabidopsis thaliana (Mouse-ear | 407 . . . 626 | 100/249 (39%) | |
| | cress), 839 aa. | | | |
| Q41805 | EXTENSIN-LIKE PROTEIN | 492 . . . 848 | 88/361 (24%) | 5e−12 |
| | PRECURSOR-Zea mays | 415 . . . 749 | 124/361 (33%) | |
| | (Maize), 1188 aa. | | | |

PFam analysis predicts that the NOV50a protein contains the domains shown in the Table 50E.

TABLE 50E
Domain Analysis of NOV50a

| | | | |
|---|---|---|---|
| | | Identities/ | |
| Pfam | NOV50a | Similarities for | Expect |
| Domain | Match Region | the Matched Region | Value |

No Significant Matches Found

### Example 51

The NOV51 clone was analyzed, and the nucleotide and predicted polypeptide sequences are shown in Table 51A.

TABLE 51A
NOV51 Sequence Analysis

| | SEQ ID NO:155 | 1394 bp | |
|---|---|---|---|
| NOV51, | GTGGCCTGCTCCTGCAGCAATCCCAGGACCCCCTGCTCATGGGGCTGTTTCCTACTAA | | |
| CG59492-01 DNA Sequence | | | |
| | CCCCAAAGAGAAGACCCAGGAGGAACCCCCTGGCCAGAGCAGGGCCCCTGTGTTGACC | | |
| | GTGGTGTCCAAGTTCAAGGCCTCACTGGAGCAGCTTCTGCAGGTCCTACACAGCACCA | | |
| | CGCCCCACTACATTCGCTGCATCAAGCCCAACAGCCAGGGCCAGGCGCAGACCTTTCT | | |
| | CCAAGAGGAGGTCCTGAGCCAGCTGGAGGCCTGTGGCCTCGTGGAGACCATCCATATC | | |
| | AGTGCTGCTGGCTTCCCCATCCGGGTCTCTCACCGAAACTTTGTAGAACGATACAAGT | | |
| | TACTAAGAAGGCTTCATCCTTGCACATCCTCTGGCCCCGACAGCCCATATCCTGCCAA | | |
| | AGGGCTCCCTGAATGGTGTCCACACAGCGAGGAAGCCACGCTTGAACCTCTCATCCAG | | |
| | GACATTCTCCACACTCTGCCGGTCCTAACTCAGGCAGCAGCCATAACTGGTGACTCGG | | |
| | CTGAGGCCATGCCAGCCCCCATGCACTGTGGCAGGACCAAGGTGTTCATGACTGACTC | | |
| | TATGCTGGAGCTTCTGGAATGTGGGCGTGCCCGGGTGCTGGAGCAGTGTGCCCGCTGC | | |
| | ATCCAGGGTGGCTGGAGGCGACACCGGCACCGAGAGCAGGAGCGGCAGTGGCGGGCCG | | |
| | TCATGCTCATCCAGGCAGCCATTCGTTCCTGGTTAACTCGGAAACACATCCAGAGGCT | | |
| | GCATGCAGCTGCCACAGTCATCAAGCGTGCATGGCAGAAGTGGAGAATCAGAATGGCC | | |
| | TGCCTTGCTGCTAAAGAGCTGGATGGTGTGGAAGAAAAACACTTCTCTCAAGCTCCCT | | |
| | GTTCCCTGAGCACCTCGCCGCTGCAGACCAGGCTCCTGGAGGCAATAATCCGCTTCTG | | |
| | GCCCCTGGGACTGGTCCTGGCCAATACGGCTATGGGTGTAGGCAGCTTTCAGAGGAAA | | |
| | TTAGTGGTCTGGGCTTGCCTCCAGCTCCCCAGGGGCAGCCCCAGTAGCTACACTGTCC | | |
| | AGACAGCACAAGACCAGGCTGGTGTCACGTCCATCCGAGCGCTGCCTCAGGGATCGAT | | |
| | AAAGTTTCACTGCAGAAAGTCTCCACTGCGGTATGCTGACATCTGCCCTGAACCTTCA | | |
| | CCCTACAGCATTGCAGGCTTTAATCAGATTCTGCTGGAAAGACACAGGCTGATCCACG | | |
| | TGACCTCTTCTGCCTTCACTGGGCTGGGGTGATCCTTGGTGCCTTTGTTTCCACAAGG | | |
| | CCTTTTCCTGCCCCCTGCCTTGCCAAAGACATTTAATCAGCACACAGCTGCCAGACTA | | |
| | TTCCCACAGTGCTCCAAATGCACATGAACAACAGTGACGGCTCCAGCCTTCGACCCAG | | |
| | AG | | |
| | ORF Start: ATG at 39 | | ORF Stop: TGA at 1248 |
| | SEQ ID NO:156 | 403 aa | MW at 45142.8 kD |

| | |
|---|---|
| NOV51a, | MGLFPTNPKEKTQEEPPGQSRAPVLTVVSKFKASLEQLLQVLUSTTPHYIRCIKPNSQ |
| CG59492-01 Protein Sequence | |
| | GQAQTFLQEEVLSQLEACGLVETIHISAAGFPIRVSHRNFVERYKLLRRLHPCTSSGP |
| | DSPYPAKGLPEWCPHSEEATLEPLIQDILHTLPVLTQAAAITGDSAEAMPAPMHCGRT |
| | KVFMTDSNLELLECGRARVLEQCARCIQGGWRRHRHREQERQWRAVMLIQAAIRSWLT |
| | RKHIQRLHAAATVIKRAWQKWRIRMACLAAKELDGVEEKHFSQAPCSLSTSPLQTRLL |
| | EAIIRFWPLGLVLANTAMGVGSFQRKLVVWACLQLPRGSPSSYTVQTAQDQAGVTSIR |
| | ALPQGSIKFIICRKSPLRYADICPEPSPYSIAGFNQILLERHRLIHVTSSAFTGLG |

Further analysis of the NOV51a protein yielded the following properties shown in Table 51B.

TABLE 51B
Protein Sequence Properties NOV51a

| | |
|---|---|
| PSort | 0.3000 probability located in nucleus; 0.2029 probability |
| analysis: | located in lysosome (lumen); 0.1000 probability located |
| | in mitochondrial matrix space; 00320 probability located |
| | in microbody (peroxisome) |
| SignalP | No Known Signal Sequence Predicted |
| analysis: | |

A search of the NOV51a protein against the Geneseq database, a proprietary database that contains sequences published in patents and patent publication, yielded several homologous proteins shown in Table 51C.

TABLE 51C
Geneseq Results for NOV51a

| | | NOV51a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAY94290 | Human myosin heavy chain | 1 . . . 403 | 401/403 (99%) | 0.0 |
| | homologue-Homo sapiens, 612 | 210 . . . 612 | 401/403 (99%) | |
| | aa. [WO200026372-A1, | | | |
| | 11 MAY 2000] | | | |
| AAU23676 | Novel human enzyme polypeptide | 17 . . . 403 | 384/387 (99%) | 0.0 |
| | #762-Homo sapiens, 387 aa. | 1 . . . 387 | 384/387 (99%) | |
| | [WO200155301-A2, | | | |
| | 2 AUG. 2001] | | | |
| ABB10243 | Human cDNA SEQ ID NO: 551- | 1 . . . 365 | 365/365 (100%) | 0.0 |
| | Homo sapiens, 570 aa. | 206 . . . 570 | 365/365 (100%) | |
| | [WO200154474-A2, | | | |
| | 2 AUG. 2001] | | | |
| AAU23123 | Novel human enzyme polypeptide | 1 . . . 365 | 364/365 (99%) | 0.0 |
| | #209-Homo sapiens, 567 aa. | 203 . . . 567 | 364/365 (99%) | |
| | [WO200155301-A2, | | | |
| | 2 AUG. 2001] | | | |
| AAM23563 | Human EST encoded protein SEQ | 1 . . . 189 | 188/189 (99%) | e−108 |
| | ID NO: 1088-Homo sapiens, 477 | 288 . . . 476 | 188/189 (99%) | |
| | aa. [WO200154477-A2, | | | |
| | 2 AUG. 2001] | | | |

In a BLAST search of public sequence databases, the NOV51a protein was found to have homology to the proteins shown in the BLASTP data in Table 5 ID.

TABLE 51D
Public BLASTP Results for NOV51a

| | | NOV51a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96H55 | HYPOTHETICAL 86.7 KDA | 72 . . . 403 | 330/332 (99%) | 0.0 |
| | PROTEIN-Homo sapiens (Human) | 439 . . . 770 | 330/332 (99%) | |
| | 770 aa. | | | |
| Q9D2Z3 | 1110055A02R1K PROTEIN | 3 . . . 394 | 288/394 (73%) | e−162 |
| | RIKEN CDNA 1110055A02 | 2 . . . 395 | 320/394 (81%) | |
| | GENE)-Mus musculus (Mouse), | | | |
| | 395 aa. | | | |
| Q948A2 | PUTATIVE MYOSIN HEAVY | 2 . . . 255 | 84/258 (32%) | 1e−23 |
| | CHAIN-Oryza sativa (Rice), 1601 | 663 . . . 876 | 125/258 (47%) | |
| | aa. | | | |
| O74805 | HYPOTHETICAL MYOSIN- | 20 . . . 347 | 96/340 (28%) | 1e−21 |
| | LIKE PROTEIN C2D10.14C IN | 615 . . . 903 | 152/340 (44%) | |
| | CHROMOSOME II- | | | |
| | Schizosaccharomyces pombe | | | |
| | (Fission yeast), 1471 aa. | | | |
| T30148 | hypothetical protein E02C12.1- | 5 . . . 249 | 74/248 (29%) | 6e−21 |
| | Caenorhabditis elegans, 1019 aa. | 619 . . . 830 | 119/248 (47%) | |

PFam analysis predicts that the NOV51a protein contains the domains shown in the Table 51E.

TABLE 51E
Domain Analysis of NOV51a

| | | Identities/ | |
|---|---|---|---|
| Pfam | NOV51a | Similarities for | Expect |
| Domain | Match Region | the Matched Region | Value |
| myosin_head: domain 1 | 26 . . . 105 | 37/81 (46%) | 5.1e−25 |
| of 1 | | 60/81 (74%) | |

### Example 52

The NOV52 clone was analyzed, and the nucleotide and predicted polypeptide sequences are shown in Table 52A.

TABLE 52A
NOV52 Sequence Analysis

| | SEQ ID NO:157 | 1380 bp | |
|---|---|---|---|
| NOV52a, | TAGAATTCCAGCGGCCGCTGAAATCCTCACTCGGTCAGTTCCTCGGGCGAGTTACGGG | | |
| CG59564-01 DNA Sequence | | | |
| | GACGACCTGCGGGAGCACGCGGGCAGTGGCCGGACGCTGAAGCCCAGGAGAGCGATGG | | |
| | AGACGTATGCGGAGGTTGGGAAGGAGGGCAAGCCTTCCTGTGCATCGGTGGATCTGCA | | |
| | GGGAGACAGCTCCTTACAGGTGGAGATTTCTCACGCAGTGAGTGAGCGGGACAAGGTG | | |
| | AAATTCACTGTTCAAACAAAGAGCTGCCTCCCTCACTTCGCCCAGACCGAGTTCTCAG | | |
| | TCGTGCGGCAGCACGAGGAGTTCATCTGGCTGCATGATGCCTACGTGGAGAATGAGGA | | |
| | GTACGCCGGCCTCATCATCCCCCCAGCCCCTCCGAGCCCAGACTTTGAGGCTTCGAGG | | |
| | GAAAAGCTACAGAAATTGGGCGAGGGGGACAGCTCTGTCACTCGGGAAGAGTTTGCCA | | |
| | AGATGAAGCAGGAGCTGGAAGCGGAGTACCTGGCCATCTTTAAGAAGACAGTTGCGAT | | |
| | GCACGAAGTCTTTCTGCAGCGCCTGGCGGCCCACCCCACCCTGCGTCGAGACCACAAC | | |
| | TTCTTTGTGTTTTTGCAATATGGACAGGATCTCAGTGTCCGGGGGAAGAACAGGAAGG | | |
| | AGCTCCTCGGAGGGTTTCTGAGGAATATTGTGAAGTCCGCGGATGAAGCCCTCATCAC | | |
| | GGGCATGTCAGGGCTCAAGGAGGTGGATGACTTCTTTGAGCATGAGAGGACCTTCCTG | | |
| | TTGGAGTATCACACCCGTATCCGAGATGCCTGCCTCCGGGCCGACCGCGTCATGCGCG | | |
| | CCCACAAGTGCCTGGCAGACGATTATATCCCTATCTCAGCTGCGCTGAGCAGTCTGGG | | |
| | AACACAGGAAGTCAACCAGCTAAGGACGAGCTTCCTCAAATTGGCACAGCTCTTTGAC | | |
| | CGGCTGAGGAAGCTGGAGGGCCGGGTGGCTTCCGATGAGGACCTGAAGCTGTCAGACA | | |
| | TGCTGAGGTACTACATGCGTGACTCACAGGCAGCCAAGGACCTGCTGTACCGGCGGCT | | |
| | GCGGGCACTGGCCGACTACGACAATGCCAACAAGGCGCTGCACAAGCCGCGCACCAGG | | |
| | AACCGGGAGGTGCGGCCCGCCGAGAGCCACCAGCAGCTGTGCTGCCAACGCTTCGAGC | | |
| | GCCTCTCCGACTCCGCCAAGCAAGAGCTCATGGACTTCAAGTCCCGCCGGGTCTCCTC | | |
| | TTTTCGAAAGAATCTCATTGAGCTGGCAGAGCTCGAGCTCAAACACGCCAAGGCCAGC | | |
| | ACCCTGATTCTCCGGAACACCCTTGTTGCCCTAAAGGGGGAGCCTTAGAGTAGCCAGA | | |
| | GCTCAGCCAGACCCTAATCTGGGATCTCCAGTCACCAGGGTATCCC | | |
| | ORF Start: ATG at 113 | | ORF Stop: TAG at 1322 |
| | SEQ ID NO:158 | 403 aa | MW at 46384.2 kD |

| | |
|---|---|
| NOV52a, | METYAEVGKEGKPSCASVDLQGDSSLQVEISDAVSERDKVKFTVQTKSCLPHFAQTEF |
| CG59564-01 Protein Sequence | |
| | SVVRQHEEFIWLHDAYVENEEYAGLIIPPAPPRPDFEASREKLQKLGEGDSSVTREEF |
| | AKMKQELEAEYLAIFKKTVAMHEVFLQRLAAHPTLRRDHNFFVFLEYGQDLSVRGKNR |
| | KELLGGFLRNIVKSADEALITGMSGLKEVDDFFFHERTFLLEYHTRTRDACLRADRVN |
| | RAHKCLADDYIPISAALSSLGTQEVNQLRTSFLKLAELFDRLRKLEGRVASDEDLKLS |
| | DMLRYYMRDSQAAKDLLYRRLRALADYENANKALDKARTRNREVRPAESEQQLCCQRF |
| | ERLSDSAKQELMDFKSRRVSSFRKNLIELAELELKHAKASTLILRNTLVALKGEP |

Further analysis of the NOV52a protein yielded the following properties shown in Table 52B.

TABLE 52B
Protein Sequence Properties NOV52a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV52a protein against the Geneseq database, a proprietary database that contains sequences published in patents and patent publication, yielded several homologous proteins shown in Table 52C.

TABLE 52C
Geneseq Results for NOV52a

| | | NOV52a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAY94209 | Human TRAF four associated factor | 17 . . . 402 | 273/386 (70%) | e−160 |
| | TFAF2-Homo sapiens, 406 aa. | 23 . . . 405 | 333/386 (85%) | |
| | [CA2245340-A1, 19 FEB. 2000] | | | |
| AAB07856 | Amino acid sequence of Smad1 | 17 . . . 402 | 273/386 (70%) | 1e−160 |
| | interactor protein clone S1 + 12-2- | 31 . . . 413 | 333/386 (85%) | |
| | Homo sapiens, 414 aa. | | | |
| | [WO200047102-A2, 17 AUG. 2000] | | | |
| AAB43157 | Human ORFX ORF2921 | 17 . . . 402 | 273/386 (70%) | e−160 |
| | polypeptide sequence SEQ ID | 77 . . . 459 | 333/386 (85%) | |
| | NO: 5842-Homo sapiens, 460 aa. | | | |
| | [WO200058473-A2, 5 OCT. 2000] | | | |
| AAB58368 | Lung cancer associated polypeptide | 17 . . . 402 | 273/386 (70%) | e−160 |
| | sequence SEQ ID 706-Homo | | | |
| | sapiens, 414 aa. [WO200055180- | | | |
| | A2, 21 SEP. 2000] | | | |
| AA013507 | Human polypeptide SEQ ID NO- | 17 . . . 400 | 242/384 (63%) | e−144 |
| | 27399-Homo sapiens, 443 aa. | 61 . . . 441 | 317/384 (82%) | |
| | [WO200164835-A2, 7 SEP. 2001] | | | |

In a BLAST search of public sequence databases, the NOV52a protein was found to have homology to the proteins shown in the BLASTP data in Table 52D.

TABLE 52D
Public BLASTP Results for NOV52a

| | | NOV52a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9UNH7 | Sorting nexin 6 (TRAF4-associated | 17 . . . 402 | 273/386 (70%) | e−159 |
| | factor 2)-Homo sapiens (Human), | 23 . . . 405 | 333/386 (85%) | |
| | 406 aa. | | | |
| Q9CZ03 | 2810425K19RIK PROTEIN-Mus | 17 . . . 402 | 271/386 (70%) | e−159 |
| | musculus (Mouse), 406 aa. | 23 . . . 405 | 333/386 (86%) | |
| Q9Y5X3 | Sorting nexin 5-Homo sapiens | 17 . . . 400 | 242/384 (63%) | e−143 |
| | (Human), 404 aa. | 22 . . . 402 | 317/384 (82%) | |
| Q9D8U8 | Sorting nexin 5-Mus musculus | 17 . . . 400 | 241/384 (62%) | e−142 |
| | (Mouse), 404 aa. | 22 . . . 402 | 314/384 (81%) | |
| Q96NG4 | CDNA FLJ30934 FIS, CLONE | 1 . . . 237 | 236/237 (99%) | e−134 |
| | FEBRA2007017, MODERATELY | 1 . . . 237 | 236/237 (99%) | |
| | SIMILAR TO HOMO SAPIENS | | | |
| | TRAF4-ASSOCIATED FACTOR 2 | | | |
| | MRNA-Homo sapiens (Human), | | | |
| | 277 aa. | | | |

PFam analysis predicts that the NOV52a protein contains the domains shown in the Table 52E.

TABLE 52E
Domain Analysis of NOV52a

| | | Identities/ | |
|---|---|---|---|
| Pfam | NOV52a | Similarities for | Expect |
| Domain | Match Region | the Matched Region | Value |
| PX: domain 1 of 1 | 23 . . . 164 | 39/160 (24%) | 1.6e−15 |
| | | 103/160 (64%) | |

### Example 53

The NOV53 clone was analyzed, and the nucleotide and predicted polypeptide sequences are shown in Table 53A.

TABLE 53A
NOV53 Sequence Analysis

| | SEQ ID NO:159 | 3056 bp | |
|---|---|---|---|
| NOV53a, | CTCCTGCGGGGTCAAATACAGAATTTACGCACCCTTCGCTTCCTTGGAGCCTAGCGGC | | |
| CG59553-01 DNA Sequence | | | |
| | TCTCCCCGCGTCCAAGATGGCGGCAGAAGCAGCTGGTGGGAAATACAGAAGCACAGTC | | |
| | AGCAAAAGCAAAGACCCCTCGGCGCTGCTCATCTCTCTGATCAGGACTCTGTCTACTA | | |
| | GTGACGATGTCGAAGACAGGGAAAATGAAAAGGGTCGCCTTGAAGAAGCCTACGAGAA | | |
| | ATGTGACCGTGACCTGGATGAATTGATTGTACAGCACTACACAGAATTGACGACAGCC | | |
| | ATTCGCACATACCAGAGCATCACAGAGCGCATCACTAACTCCCGAAATAAAATAAAGC | | |
| | AGGTAAAAGAGAACCTGCTTTCATGCAAGATCCTGCTGCACTGCAAACCGGATGAGCT | | |
| | TCGGAAACTGTGCATTGAAGGAATTGAGCATAAGCATGTCCTGAACTTGTTGGATGAA | | |
| | ATTGAGAATATCAAGCAAGTGCCTCAAAAGCTGGAACAGTGCATGGCCAGCAAGCACT | | |
| | ATCTCAGTGCCACTGACATGTTGGTGTCAGCAGTTGAGTCTTTGGAGGGCCCCCTGCT | | |
| | CCACGTCGAAGGACTCAGTGACCTTCGACTACAGCTTCACAGCAAGAAGATGAACCTT | | |
| | CACTTGGTTCTCATAGATGAACTACACCGGCACCTGTACATCAAATCGACTAGCCGAG | | |
| | TTGTGCAGCGTAACAAGGAAAAAGGGAAAATCAGCTCCCTCGTGAAACATCCTTCTGT | | |
| | TCCTCTGATTGATGTTACAAACCTCCCTACTCCTCGAAAATTCCTTGATACCTCTCAC | | |
| | TATTCTACTGCTGGAAGCTCAAGTGTGAGGGAGATAAATCTGCAGGACATCAAGGAAG | | |
| | ATTTAGAATTGGATCCAGAGGAAAACAGCACCCTGTTTATGGGTATCCTCATTAAGGG | | |
| | CTTGGCGAAACTGAAGAAGATCCCAGAAACAGTTAAGGCAATCATAGAGCGCTTGGAG | | |
| | CAGGAGTTGAAGCAAATTCTGAAGAGGTCTACAACCCACGTGCCAGACAGTGGCTATC | | |
| | AGCGGCGGGAGAACGTTACTGTGGAGAACCAACCAAGGTTGCTTCTAGAACTGCTGGA | | |
| | GTTACTGTTTGACAAGTTTAATGCTGTAGCCGCTGCACACTCTGTGGTCCTGGGATAC | | |
| | CTGCAGGACACTGTAGTGACTCCACTGACTCAGCAGGAAGATATCAAACTGTATGATA | | |
| | TGGCAGATGTATCGGTCAAGATCCAAGATGTTCTACAGATGCTATTAACTCAGTACTT | | |
| | GGATATGAAAAATACTCGTACGGCCTCTGAACCATCAGCTCAACTAAGCTATGCCAGC | | |
| | ACTGGACGAGAGTTTGCACCCTTTTTTGCCAAGAACAAACCTCAAACGCCAAAAAATT | | |
| | CTCTTTTCAAGTTCGAATCGTCCTCCCATGCCATCAGTATGAGCGCCTATCTGCGAGA | | |
| | ACAGAGAAGGGAGCTCTATAGTCGGAGTGGACAACTCCAAGGCGGTCCTCATGACAAC | | |
| | TTAATTGAAGGTGGAGGAACAAAATTTGTCTGCAAACCTGGAGCCAGAAACATTACCG | | |
| | TCATATTCCACCCATTACTAAGATTTATTCAGGAGATTGAGCATGCTCTGCGTCTTGG | | |
| | CCCAGCCAAACAGTGTCCTGTTCGAGAGTTTCTCACCGTGTACATCAAAAACATCTTT | | |
| | CTCAATCAAGTCTTGCCTGAGATCAACAAGCAGATTGAACGAGTCACTAAAACATCTG | | |
| | ACCCTTTGAAGATTCTCGCCAACGCACACACCATGAAGGTGCTGGCACTGCAGCGGCC | | |
| | TCTCCTACAGAGCACAATCATTGTGGAGAAGACAGTTCAAGACCTCCTGAACCTGATG | | |
| | CATGACTTGAGTGCATATTCAGATCAATTCCTCAACATGGTGTGCGTGAAGCTCCAGG | | |
| | AGTACAAGGACACCTGCACTGCAGCTTACAGGGGTATTGTCCAGTCAGAAOAAAAACT | | |
| | TGTCATCACTCCATCCTGGGCAAAAGATGATGATATCAGCAGACTCTTGAAATCTCTA | | |
| | CCAAACTGGATGAATATGGCTCAACCCAAACAGCTGAGGCCAAAAAGAGAGGAGGAAG | | |
| | AAGATTTCATAAGGGCAGCTTTTGGCAAGGAGTCTGAAGTTCTTATTGGGAACCTGGG | | |
| | TGATAAATTAATCCCTCCACAAGACATCCTTCCTGACCTCAGTGACCTCAAAGCCTTG | | |
| | GCCAACATGCATGAAAGCCTGGAATGCTTGGCAACTCGAACAAAGTCAGCTTTCTCCA | | |
| | ATCTTTCTACATCCCAGATGCTTTCTCCTGCTCAAGACAGCCACACGAACACGGATCT | | |
| | CCCCCCAGTGTCAGAGCAGATCATGCAGACTCTCAGTGAACTTGCCAAATCGTTCCAG | | |
| | GATATGGCTGACCGCTGCTTGCTTGTCTTACATCTCGAAGTGACGGTTCACTGTTTCC | | |
| | ACTATCTTATCCCTCTTGCAAAGGAGGGGAACTATGCCATTGTGGCTAATGTGGAAAG | | |
| | TATGGATTATGACCCCCTGGTGGTCAAGGTCAACAAAGATATCAGCGCCATTGAAGAG | | |
| | GCCATGAGCGCCAGCCTTCAGCAGCACAAGTTCCAGTATATCTTCGAAGGCCTGGGCC | | |
| | ACCTGATCTCCTGCATCCTCATTAATGGTGCCCAGTACTTCAGGCGCATCAGTGAGTC | | |
| | TGGCATCAAGAAAATGTGTAGGAACATTTTTGTTCTTCAGCAGAATTTGACCAACATC | | |
| | ACCATGTCGCCGGAGGCAGACCTGGACTTTGCAAGGCAGTACTACGAGATGCTTTACA | | |
| | ACACAGCTGACGAGCTCCTGAACCTGGTGGTGGACCAGGGTGTGAAGTACACGGAGCT | | |
| | GGAGTACATCCACGCTCTGACCCTGCTGCACCGCAGCCAGACTGGGGTGGGGGAACTG | | |
| | ACCACCCAGAACACGAGCTGCAGAGGAGGCTCAAAGAGATCATCTGCGAGCAGGCTGC | | |
| | CATCAAGCAAGCCACCAAGGACAAGAAGATAACTACCGTTTAGCAGGGCGTACTGCGG | | |
| | TTGGTGACGGGGGTCCCCTCAGTCACACTCACTTTTTTCC | | |
| | ORF Start: ATG at 75 | | ORF Stop: TAA at 2988 |
| | SEQ ID NO:160 | 971 aa | MW at 109984.9 kD |

| | |
|---|---|
| NOV53a, | MAAEAAGGKYRSTVSKSKDPSGLLISVIRTLSTSDDVEDRENEKGRLEEAYEKCDRDL |
| CG59553-01 Protein Sequence | |
| | DELIVQHYTELTTAIRTYQSITERITNSRNKIKQVKENLLSCKMLLHCKRDELRKLWI |
| | EGIEHKHVLNLLDEIENIKQVPQKLEQCMASKHYLSATDMLVSAVESLEGPLLQVEGL |
| | SDLRLELHSKKMNLHLVLIDELRRMLYIKSTSRVVQRNKEKGKISSLVRDASVPLIDV |
| | TNLPTPRKFLDTSHYSTAGSSSVREINLQDIKEDLELDPEENSTLFMGILIKGLAKLK |
| | KIPETVKAITERLEQELKQIVKRSTTQVADSGYQRGENVTVENQPRLLLELLELLFDK |
| | FNAVAAAHSVVLGYLQDTVVTPLTQQEDIKLYDMADVWVKIQDVLQMLLTEYLDMKNT |
| | RTASEPSAQLSYASTGREFAAFFAKKKPQRPKNSLFKEESSSHAISMSAYLREQRREL |
| | YSRSGELQCCPDDNLTECCGTKFVCKPGAPNTTVTFHPLLRFIQETEHALGLCPAKQC |
| | PLREFLTVYIKNIFLNQVLAEINKEIEGVTKTSDPLKILANADTMKVLGVQRPLLQST |
| | IIVEKTVQDLLNLMHDLSAYSDQFLNMVCVKLQEYKDTCTAAYRGIVQSEEKLVISAS |
| | WAKDDDISRLLKSLPNWMNMAQPKQLRPKREEEEDFIRAAFGKESEVLIGNLGDKLIP |
| | PQDILRDVSDLKALANMHESLEWLASRTKSAFSNLSTSQMLSPAQDSHTNTDLPPVSE |
| | QIMQTLSELAFSFQDMADRCLLVLHLEVRVHCFHYLIPLAKEGNYAIVANVESMDYDP |
| | LVVKLNKDISAIEEAMSASLQQHKFQYIFEGLGHLISCILINGAQYFRRISESGIKKM |
| | CRNIFVLQQNLTNITMSREADLDFARQYYEMLYNTADELLNLVVDQGVKYTELEYIHA |
| | LTLLHRSQTGVCELTTQNTSCRCGSKRSSASRLPSSKPPRTRR |

Further analysis of the NOV53a protein yielded the following properties shown in Table 53B.

TABLE 53B
Protein Sequence Properties NOV53a

| | |
|---|---|
| PSort | 0.5500 probability located in endoplasmic reticulum |
| analysis: | (membrane); 0.1900 probability located in lysosome |
| | (lumen); 0.1000 probability located in endoplasmic |
| | reticulum (lumen); 0.1000 probability located in outside |
| SignalP | No Known Signal Sequence Predicted |
| analysis: | |

A search of the NOV53a protein against the Geneseq database, a proprietary database that contains sequences published in patents and patent publication, yielded several homologous proteins shown in Table 53C.

TABLE 53C
Geneseq Results for NOV53a

| | | NOV53a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB93175 | Human protein sequence SEQ ID | 1 . . . 947 | 947/947 (100%) | 0.0 |
| | NO: 12114-Homo sapiens ,974 aa. | 1 . . . 947 | 947/947 (100%) | |
| | [EP1074617-A2, 7 FEB. 2001] | | | |
| AAW69801 | Amino acid sequence of rsec8, a | 1 . . . 947 | 902/948 (95%) | 0.0 |
| | protein present in SA-17S | 1 . . . 948 | 925/948 (97%) | |
| | complex-Rattus sp, 975 aa. | | | |
| | [WO9828419-A2, 2 JUL. 1998] | | | |
| AAB95143 | Human protein sequence SEQ ID | 403 . . . 947 | 545/545 (100%) | 0.0 |
| | NO: 17163-Homo sapiens, 572 aa. | 1 . . . 545 | 545/545 (100%) | |
| | [EP1074617-A2, 7 FEB. 2001] | | | |
| AAB58175 | Lung cancer associated | 571 . . . 947 | 369/377 (97%) | 0.0 |
| | polypeptide sequence SEQ ID 513- | 15 . . . 391 | 369/377 (97%) | |
| | Homo sapiens, 418 aa. | | | |
| | [WO200055180-A2, | | | |
| | 21 SEP. 2000] | | | |
| AAG00950 | Human secreted protein, SEQ ID | 451 . . . 544 | 76/94 (80%) | 3e−36 |
| | NO: 5031-Homo sapiens, 100 aa. | 7 . . . 100 | 79/94 (83%) | |
| | [EP1033401-A2, 6 SEP. 2000] | | | |

In a BLAST search of public sequence databases, the NOV53a protein was found to have homology to the proteins shown in the BLASTP data in Table 53D.

TABLE 53D
Public BLASTP Results for NOV53a

| | | NOV53a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96A65 | CDNA FLJ14782 FIS, CLONE | 1 . . . 947 | 947/947 (100%) | 0.0 |
| | NT2RP4000524, HIGHLY | 1 . . . 947 | 947/947 (100%) | |
| | SIMILAR TO MUS MUSCULUS | | | |
| | SEC8 MRNA (SECRETORY | | | |
| | PROTEIN SEC8)-Homo sapiens | | | |
| | (Human), 974 aa. | | | |
| Q9C0G4 | KIAA1699 PROTEIN-Homo | 9 . . . 947 | 939/939 (100%) | 0.0 |
| | sapiens (Human), 966 aa | 1 . . . 939 | 939/939 (100%) | |
| | (fragment). | | | |
| O35382 | SEC8-Mus musculus (Mouse), 971 | 1 . . . 971 | 923/972 (94%) | 0.0 |
| | aa. | 1 . . . 971 | 946/972 (96%) | |
| Q62824 | RSEC8-Rattus norvegicus (Rat), | 1 . . . 947 | 902/948 (95%) | 0.0 |
| | 975 aa (fragment). | 1 . . . 948 | 925/948 (97%) | |
| Q9P102 | REC8-Homo sapiens (Human), | 339 . . . 947 | 609/609 (100%) | 0.0 |
| | 637 aa (fragment). | 2 . . . 610 | 609/609 (100%) | |

PFam analysis predicts that the NOV53a protein contains the domains shown in the Table 53E.

TABLE 53E
Domain Analysis of NOV53a

| | | | |
|---|---|---|---|
| | | Identities/ | |
| Pfam | NOV53a | Similarities for | Expect |
| Domain | Match Region | the Matched Region | Value |

No Significant Matches Found

### Example 54

The NOV54 clone was analyzed, and the nucleotide and predicted polypeptide sequences are shown in Table 54A.

TABLE 54A
NOV54 Sequence Analysis

| | SEQ ID NO:141 | 877 bp | |
|---|---|---|---|
| NOV54a, | CAACACGAGGAACAATGTCTTCTTTACCCGTGCCATACAAACTGCCTGTGTCTTTGTC | | |
| CG59545-01 DNA Sequence | | | |
| | TGTTGGTTCCTGCGTGATAATCAAACCOACACTGATCGACTCTTCTATCAACGAACCA | | |
| | CAGCTGCAGGTGGATTTCTACACTGAGATGAATGAGGACTCAGAAATTGCCTTCCATT | | |
| | TGCGAGTGCACTTAGGCCGTCGTGTGGTCATGAACAGTCGTGAGTTTGGGATATGGAT | | |
| | GTTGGAGGAGAATTTACACTATGTCCCCTTTGAGGATGGCAAACCATTTCACTTGCGC | | |
| | ATCTACGTGTGTCTCAATGAGTATGACGTAAAGGTAAATGGTGAATACATTTATGCCT | | |
| | TTGTCCATCGAATCCCGCCATCATATGTGAAGATGATTCAAGTCTGGAGAGATCTCTC | | |
| | CCTGGACTCAGTGCTTGTCAACAATGGACGGAGATGATCACACTCCTCATTGTTGAGG | | |
| | AAACCCTCTTTCTACCTCACCATCGGATTCCTAGAGC | | |
| | ORF Start: ATG at 15 | | ORF Stop: TGA at 441 |
| | SEQ ID NO:162 | 142 aa | MW at 16511.9 kD |

| | |
|---|---|
| NOV54a, | MSSLPVPYKLPVSLSVGSCVIIKGTLIDSSINEPQLQVDFYTEMNEDSEIAFHLRVIL |
| CG59545-01 Protein Sequence | |
| | GRRVVMNSREFGIWMLEENLHYVPFEDGKPFDLRIYVCLNEYEVKVNGEYIYAFVHRI |
| | PPSYVKMTQVWRDVSLDSVLVNNGRR |

Further analysis of the NOV54a protein yielded the following properties shown in Table 54B.

TABLE 54B
Protein Sequence Properties NOV54a

| | |
|---|---|
| PSort | 0.5500 probability located in endoplasmic reticulum |
| analysis: | (membrane); 0.1900 probability located in lysosome |
| | (lumen); 0.1000 probability located in endoplasmic |
| | reticulum (lumen); 0.1000 probability located |
| | in outside |
| SignalP | No Known Signal Sequence Predicted |
| analysis: | |

A search of the NOV54a protein against the Geneseq database, a proprietary database that contains sequences published in patents and patent publication, yielded several homologous proteins shown in Table 54C.

TABLE 54C
Geneseq Results for NOV54a

| | | NOV54a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAG66741 | Human Charcot-Leyden crystal | 1 . . . 142 | 139/142 (97%) | 2e−77 |
| | protein 5A (CLC5A) - Homo | 1 . . . 142 | 139/142 (97%) | |
| | sapiens, 142 aa. [CN1302875-A, | | | |
| | 11 JUL 2001] | | | |
| AAG66742 | Human Charcot-Leyden crystal | 6 . . . 142 | 136/137 (99%) | 3e−76 |
| | protein 5B (CLC5B) - Homo | 34 . . . 170 | 136/137 (99%) | |
| | sapiens, 170 aa. [CN1302875-A, | | | |
| | 11 JUL 2001] | | | |
| AAM79041 | Human protein SEQ ID NO 1703 - | 1 . . . 139 | 107/139 (76%) | 2e−56 |
| | Homo sapiens, 139 aa. | 1 . . . 139 | 116/139 (82%) | |
| | [WO200157190-A2, 09 AUG | | | |
| | 2001] | | | |
| AAY28350 | Full Placental Protein 13 amino | 1 . . . 139 | 107/139 (76%) | 2e−56 |
| | acid sequence - Homo sapiens, 139 | 1 . . . 139 | 116/139 (82%) | |
| | aa. [WO9938970-A1, 5 AUG | | | |
| | 1999] | | | |
| AAG78627 | Human Charcot-Leyden crystal 4 | 6 . . . 139 | 102/134 (76%) | 2e−53 |
| | CLC4 protein #2 - Homo sapiens, | 34 . . . 167 | 111/134 (82%) | |
| | 167 aa. [CN1302876-A, 11 JUL | | | |
| | 2001] | | | |

In a BLAST search of public sequence databases, the NOV54a protein was found to have homology to the proteins shown in the BLASTP data in Table 54D.

TABLE 54D
Public BLASTP Results for NOV54a

| | | NOV54a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9UHV8 | PLACENTAL PROTEIN 13 | 1 . . . 139 | 107/139 | (76%) | 9e−56 |
| | (PLACENTA PROTEIN 13) - Homo | 1 . . . 139 | 116/139 | (82%) | |
| | sapiens (Human), 139 aa. | | | | |
| Q9NR03 | PLACENTAL PROTEIN 13-LIKE | 1 . . . 139 | 86/139 | (61%) | 9e−45 |
| | PROTEIN - Homo sapiens (Human), | 1 . . . 139 | 107/139 | (76%) | |
| | 139 aa. | | | | |
| A46523 | Charcot-Leyden crystal protein - | 1 . . . 142 | 76/142 | (53%) | 7e−36 |
| | human, 142 aa. | 1 . . . 142 | 96/142 | (67%) | |
| Q05315 | Eosinophil lysophospholipase (EC | 2 . . . 142 | 75/141 | (53%) | 3e−35 |
| | 3.1.1.5) (Charcot-Leyden crystal | 1 . . . 141 | 95/141 | (67%) | |
| | protein) (Lysolecithin acylhydrolase) | | | | |
| | (CLC) (Galactin-10) - Homo sapiens | | | | |
| | (Human), 141 aa. | | | | |
| Q96KD6 | PLACENTAL PROTEIN 13-LIKE - | 1 . . . 104 | 66/104 | (63%) | 1e−31 |
| | Homo sapiens, (Human), 104 aa | 1 . . . 104 | 79/104 | (75%) | |
| | (fragment). | | | | |

PFam analysis predicts that the NOV54a protein contains the domains shown in the Table 54E.

TABLE 54E
Domain Analysis of NOV54a

| | | Identities/ | | |
|---|---|---|---|---|
| | | Similarities | | |
| | NOV54a Match | for the Matched | | Expect |
| Pfam Domain | Region | Region | | Value |
| Gal-bind_lectin: | 5 . . . 137 | 37/142 | (26%) | 3.1e−28 |
| domain 1 of 1 | | 106/142 | (75%) | |

### Example 55

The NOV55 clone was analyzed, and the nucleotide and predicted polypeptide sequences are shown in Table 55A.

TABLE 55A
NOV55 Sequence Analysis

| | SEQ ID NO:163 | 2071 bp | |
|---|---|---|---|
| NOV55a, | AAACTATTTTTAGGCGCACTCATGCAGGAAAACCTCAGATTTCCTTCATCAGGAGATG | | |
| CG59435-01 DNA Sequence | | | |
| | ATATTAAAATATGGGATGCTTCATCTATCACATTGGTGGATAAATTCAACCCACACAC | | |
| | ATCACCACATGGAATCAGCTCAATATGTTCGA~CAGCAATAGTAACTTTTTAGTAACA | | |
| | GCATCTTCCAGTGGCGACAAAATAGTTGTCTCAAGTTGCAAATGTAAACCTGTTCCAC | | |
| | TTTTAGAGCTTGCTGAAGGGCAAAACCAGACATGTGTCAATTTAAATTCTACATCTAT | | |
| | GTATTTGGTAAGCGGAGGCCTAAATAACACTGTTAATATTTGGGATTTAAAATCAAAA | | |
| | AGAGTTCATCGATCTCTTAAGGATCATAAAGATCAAGTAACTTGTGTAACATACAATT | | |
| | GGAATCATTGCTACATTGCTTCTGGATCTCTTAGTGGTGAAATTATTTTACACAGTGT | | |
| | AACCACTAATTTATCTAGTACTCCTTTTGGCCATGGTAGTAACCAGGTTCGGCACTTG | | |
| | AAGTACTCCTTGTTTAAGAAATCACTACTGGGCAGTGTTTCGGATAATGGAATAGTAA | | |
| | CTCTCTGGGATGTAAATAGTCAGAGTCCATACCATAACTTTGACAGTGTACACAAAGC | | |
| | TCCAGCGTCAGGCATCTGTTTTTCTCCTGTCAATGAATTGCTCTTTGTAACCATAGGC | | |
| | TTGGATAAAAGAATCATCCTCTAmGACACTTCAAGTAAGAAGCTAGTGAAAACTTTAG | | |
| | TGGCTGACACTCCTCTAACTGCGGTAGATTTCATGCCTGATGCAGCCACTTTGGCTAT | | |
| | TGGATCTTCCCGGGGGAAAATATATCAATATGATTTAAGAATGTTGAAATCACCAGTT | | |
| | AAGACCATCAGTCCTCACAAGACATCTGTGCAGTGTATAGCATTTCAGTACTCCACTG | | |
| | TTCTTACTAAGTCAAGTTTAAATAAAGCCTCTTCAAATAACCCCACAACAGTGAACAA | | |
| | ACGAAGTGTTAATGTGAATGCTGCTAGTGGAGGAGTTCAGAATTCCGGAATTGTCAGA | | |
| | GAAGCACCTGCCACGTCCATTGCCACAGTTCTACCACAACCTATGACATCAGCTATGG | | |
| | GGAAAGGAACAGTTGCTGTTCAAGAAAAAGCAGGTTTGCCTCGAAGCATAAACACAGA | | |
| | CACTTTATCTAAGGAAACAGACAGTGCAAAAAATCAGGATTTCTCCAGCTTTGATGAT | | |
| | ACTGGGAAAAGTAGTTTAGGTGACATGTTCTCACCTATCAGAGATGATGCTGTAGTTA | | |
| | ACAAGGGAAGTGATGAGTCCATAGGCAAAGGAGATGGCTTTGACTTTCTACCGCAGTT | | |
| | GAACTCAGTGTTTCCTCCAACAAAAAATCCAGTAACTTCAAGTACTTCAGTATTGCAT | | |
| | TCTAGTCCTCTTAATGTTTTTATGGGATCTCCAGGGAAAGAGGAAAATCAAAACCGTG | | |
| | ATCTAACAGCTGAGTCTAAGAAAATATATATCGGAAAACACGAATCTAAACACTCCTT | | |
| | CAAACACTTAGCAAAGTTGGTCACATCTGGTGCTCAAAGTGGAAATCTAAATACCTCT | | |
| | CCATCATCTAACCAAACAAGAAATTCTGAGAAATTTGAAAAGCCAGAGAATGAAATTG | | |
| | AAGCCCAGTTGATATGTGAACCCCCAATCAATCGATCCTCAACTCCAAATCCAAAGAT | | |
| | AGCATCTTCTGTCACTGCTGGAGTTGCCAGTTCACTCTCAGAAAAAATAGCCGACAGC | | |
| | ATTCCAAATAACCGGCAAAATGCACCATTGACTTCCATTCAAATTCGTTTTATTCAGA | | |
| | ACATCATACAGGAAACGTTGGATGACTTTACAGAAGCATGCCATAGCGACATTGTGAA | | |
| | TTTGCAAGTGGAGATGATTAAACAGTTTCATATGCAACTCAATGAAATGCATTCTTTG | | |
| | CTGGAAAGATACTCAGTGAATGAAGGTTTAGTGGCTGAAATTGAAAGACTACGAGAAG | | |
| | AAAACAAAACATTACGGGCCCACTTTTGAAATTTCAGTGAATACCTTAATGTTCTGTA | | |
| | ATTTGGGAAGTTTCTGGCAACACAGAACTACATAGAATCAT | | |
| | ORF Start: ATG at 22 | | ORF Stop: TGA at 1999 |
| | SEQ ID NO:164 | 659 aa | MW at 71851.2 kD |
| NOV55a, | MQENLRFASSGDDIKTWDASSMTLVDKFNPETSPHGTSSTCWSSNSNFLVTASSSGDK | | |
| CG59535-01 Protein Sequence | | | |
| | IVVSSCKCKPVPLLELAEGQKQTCVNLNSTSMYLVSGCLNNTvNTwDLKSKRVERSLK | | |
| | DHKDQVTCVTYNWNDCYIASGSLSGEITLHSVTTNLSSTFFGHGSNQVRHLKYSLFKK | | |
| | SLLGSVSDNGIVTLWDVNSQSPYHNFDSVHKAPASGICFSPVNELLFVTTGLDKRIIL | | |
| | YDTSSKKLVKTLVADTPLTAVDFMPDGATLAIGSSRGKIYQYDLRMLKSPVKTISAHK | | |
| | TSVQCIAFQYSTVLTKSSLNKGCSNKPTTVNKRSVNVNAASGGVQNSCIVREAPATSI | | |
| | ATVLPQPMTSAMGKGTVAVQEKAGLPRSINTDTLSKETDSGKNQDFSSFDDTGKSSLG | | |
| | DMFSPIRDDAVVNKGSDESIGKGDGFDFLPQLNSVFPPRKNPVTSSTSVLHSSPLNVF | | |
| | MGSPGKEENENRDLTAESKKIYMGKQESKDSFKQLAKLVTSGAESGNLNTSPSSNQTR | | |
| | NSEKFEKPENEIEAQLICEPPINGSSTPNPKIASSVTAGVASSLSEKIADSIGNNRQN | | |
| | APLTSIQIRFIQNMIQETLDDFREACHRDIVNLQVEMIKQFHMQLNEMISLLERYSVN | | |
| | EGLVAEIERLREENKRLRAHF | | |
| | SEQ ID NO:165 | 2009 bp | |
| NOV55b, | AAACTATTTGTAGGCGCAGTCATGCAGGAAAACCTCAGATTTGCTTCATCAGGAGATG | | |
| CG59435-02 DNA Sequence | | | |
| | ATATTAAAATATGGGATGCTTCATCTATGACATTGGTGGATAAATTCAACCCACACAC | | |
| | ATCACCACATGGAATCACCTCAATATGTTGGACCAGCAATAATAACTTTTTAGTAACA | | |
| | GCATCTTCCAGTGGCGACAAAATAGTTGTCTCAAGTTGCAAATGTAAACCTGTTCCAC | | |
| | TTTTAGAGCTTGCTGAAGGGCAAAAGCAGACATGTGTCAATTTAAATTCTACATCTAT | | |
| | GTATTTGGTAAGCGGAGGCCTAAATAACACTGTTAATATTTGCGATTTAAAATCAAAA | | |
| | AGAGTTCATCGATCTCTTAACGATCATAAAGATCAACTAACTTGTGTAACATACAATT | | |
| | GGAATGATTCCTACATTGCTTCTGGATCTCTTAGTGGTCAAATTATTTTACACAGTGT | | |
| | AACCACTAATTTATCTAGTACTCCTTTTGGCCATGGTAGTAACCAGTCTGTTCGGCAC | | |
| | TTGAAGTACTCCTTGTTTAAGAAATCACTACTGGGCAGTGTTTCGGATAATGGAATAG | | |
| | TAACTCTCTGGGATGTAAATAGTCAGAGTCCATACCATAACTTTGACAGTCTACACAA | | |
| | AGCTCCAGCGTCAGGCATCTGTTTTTCTCCTGTCAATGAATTGCTCTTTGTAACCATA | | |
| | GCCTTGGATAAAAGAATCATCCTCTATGACACTTCAAGTAAGAAGCTAGTGAAAACTT | | |
| | TAGTGGCTGACACTCCTCTAACTGCGGTAGATTTCATGCCTGATGGAGCCACTTTGGC | | |
| | TATTGGATCTTCCCGGGGGAAAATATATCAATATGATTTAAGAATGTTGAAATCACCA | | |
| | GTTAAGACCATCAGTGCTCACAAGACATCTGTGCAGTGTATAGCATTTCAGTACTCCA | | |
| | CTGTTCTTACTAAGTCAAGTTTAAATAAAOGCTCTTCAAATAAGCCCACAACAGTGAA | | |
| | CAAACGAAGTGTTAATCTGAATGCTGCTAGTGGAGGAGTTCAGAATTCCGGAATTGTC | | |
| | AGAGAAGCACCTGCCACGTCCATTGCCACAGTTCTACCACAACCTATGACATCAGCTA | | |
| | TGGGGAAACGAACACTTGCTGTTCAAGAAAAAGCAGGTTTGCCTCGAACCATAAACAC | | |
| | AGACACTTTATCTAAGGAAACAGACAGTGGAAAAAATCAGGATTTCTCCAGCTTTGAT | | |
| | GATACTGGGAAAAGTAGTTTAGGTGACATGTTCTCACCTATCAGAGATGATGCTGTAG | | |
| | TTAACAAGGGAAGTGATGAGTCCATAGGCAAAGGAGATGGCTTTGACTTTCTACCGCA | | |
| | GTTGAACTCAGTGTTTCCTCCAAGAAAAAATCCAGTAACTTCAAGTACTTCAGTATTG | | |
| | CATTCTAGTCCTCTTAATCTTTTTATGGCATCTCCAGGGAAAGAGGAAAATGAAAACC | | |
| | GTGATCTAACAGCTGAGTCTAACAAAATATATATGGGAAAACAGGAATCTAAAGACTC | | |
| | CTTCAAACAGTTAGCAAAGTTGGTCACATCTGGTGCTGAAAGTGGAAATCTAAATACC | | |
| | TCTCCATCATCTAACCAAACAAGAAATTCTGAGAAATTTGAAAAGCCAGAGAATGAAA | | |
| | TTGAAGCCCAGTTGATATGTGAACCCCCAATCAATGGATCCTCAACTCCAAATCCAAA | | |
| | GATAGCATCTTCTGTCACTGCTGGAGTTGCCAGTTCACTCTCAGAAAAAATAGCCGAC | | |
| | AGCATTGGAAATAACCGGCAAAATGCACCATTGACTTCCATTCAAATTCGTTTTATTC | | |
| | AGAACATGATACAGGAAACGTTGCATCACTTTAGAGAAGCATGCCATAGGGACATTGT | | |
| | GAATTTGCAAGTGGAGATGATTAAACAGTTTCATATGCAACTGAATGAAATGCATTCT | | |
| | TTGCTCGAAAGATACTCAGTGAATGAAGGTTTAGTGGCTGAAATTGAAAGACTACGAG | | |
| | AAGAAAACAAAAGATTACGGGCCCACTTTTGAAATTT | | |
| | ORF Start: ATG at 22 | | ORF Stop: TGA at 2002 |
| | SEQ ID NO:166 | 600 aa | MW at 71965.3 kD |

| | |
|---|---|
| NOV55b, | MQENLRFASSGDDIKIWDASSMTLVDKFNPHTSPHGISSICWSSNNNFLVTASSSGDK |
| CG59435-02 Protein Sequence | |
| | IVVSSCKCKPVPLLELAEGQKQTCVNLNSTSMYLVSGGLNNTVNIWDLKSKRVHRSLK |
| | DHKDQVTCVTYNWNDCYIASGSLSGEIILHSVTTNLSSTPFGHGSNQSVRHLKYSLFK |
| | KSLLGSVSDNGIVTLWDVNSQSPYHNFDSVHKAPASGICFSPVNELLFVTICLDKRII |
| | LYDTSSKKLVKTLVADTPLTAVDFMPDGATLAIGSSRGKIYQYDLRMIASPVKTISAH |
| | KTSVQCIAFQYSTVLTKSSLNXGCSNKPTTVNKRSVNVNAASGGVQNSGIVREAPATS |
| | IATVLPQPMTSANGKGTVAVQEKAGLPRSINTDTLSKETDSGKNQDFSSFDDTGKSSL |
| | GDMFSPIRDDAVVNKCSDESTGKGDGFDFLPQLNSVFPPRKNPVTSSTSVLHSSPLNV |
| | FMGSPGKEENENRDLTAESKKTYMGKQESKDSFKQLAKLVTSGAESGNLNTSPSSNQT |
| | RNSEKFEKPENEIEAQLICEPPINGSSTPNPKIASSVTAGVASSLSEKIADSIGNNRQ |
| | NAPLTSIQIRFIQNNIQETLDDFREACHRDIVNLQVEMIKQFHMQLNEMHSLLERYSV |
| | NEGLVAEIERLREENRRLRAHF |

Sequence comparison of the above protein sequences yields the following sequence relationships shown in Table 55B.

TABLE 55B
Comparison of NOV55a against NOV55b.

| | | Identities/ |
|---|---|---|
| | NOV55a Residues/ | Similarities for the |
| Protein Sequence | Match Residues | Matched Region |
| NOV55b | 1 . . . 659 | 658/660 (99%) |
| | 1 . . . 660 | 659/660 (99%) |

Further analysis of the NOV55a protein yielded the following properties shown in Table 55C.

TABLE 55C
Protein Sequence Properties NOV55a

| | |
|---|---|
| PSort | 0.4500 probability located in cytoplasm; 0.3000 |
| analysis: | probability located in microbody (peroxisome); |
| | 0.1000 probability located in mitochondrial |
| | matrix space; 0.1000 probability located in |
| | lysosome (lumen) |
| SignalP | No Known Signal Sequence Predicted |
| analysis: | |

A search of the NOV55a protein against the Geneseq database, a proprietary database that contains sequences published in patents and patent publication, yielded several homologous proteins shown in Table 55D.

TABLE 55D
Geneseq Results for NOV55a

| | | | Identities/ | | |
|---|---|---|---|---|---|
| | | NOV55a | Similarities | | |
| Geneseq | Protein/Organism/Length | Residues/ | for the | | Expect |
| Identifier | [Patent #, Date] | Match Residues | Matched Region | | Value |
| AAG74568 | Human colon cancer antigen protein | 256 . . . 659 | 399/404 | (98%) | 0.0 |
| | SEQ ID NO:5332 - Homo sapiens, | 1 . . . 404 | 399/404 | (98%) | |
| | 404 aa. [WO200122920-A2, 5 | | | | |
| | APR 2001] | | | | |
| AAE10677 | Human NEDD1-related protein - | 453 . . . 611 | 145/159 | (91%) | 4e−75 |
| | Homo sapiens, 208 aa. | 2 . . . 159 | 149/159 | (93%) | |
| | [WO200172955-A2, 4 OCT 2001] | | | | |
| AAM70774 | Human bone marrow expressed | 240 . . . 306 | 67/67 | (100%) | 9e−31 |
| | probe encoded protein SEQ ID NO: | 1 . . . 67 | 67/67 | (100%) | |
| | 31080 - Homo sapiens, 67 aa. | | | | |
| | [WO200157276-A2, 9 AUG 2001] | | | | |
| AAM06190 | Peptide #4872 encoded by probe for | 240 . . . 306 | 67/67 | (100%) | 9e−31 |
| | measuring breast gene expression - | 1 . . . 67 | 67/67 | (100%) | |
| | Homo sapiens, 67 aa. | | | | |
| | [WO200157270-A2, 9 AUG 2001] | | | | |
| ABB23122 | Protein #5121 encoded by probe for | 307 . . . 371 | 65/65 | (100%) | 3e−29 |
| | measuring heart cell gene | 1 . . . 65 | 65/65 | (100%) | |
| | expression - Homo sapiens, 65 aa. | | | | |
| | [WO200157274-A2, 9 AUG 2001] | | | | |

In a BLAST search of public sequence databases, the NOV55a protein was found to have homology to the proteins shown in the BLASTP data in Table 55E.

TABLE 55E
Public BLASTP Results for NOV55a

| | | NOV55a | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| I60167 | regulatory protein Nedd1 - mouse, | 1 . . . 659 | 564/660 | (85%) | 0.0 |
| | 660 aa. | 1 . . . 660 | 607/660 | (91%) | |
| P33215 | NEDD1 protein - Mus musculus | 1 . . . 659 | 564/660 | (85%) | 0.0 |
| | (Mouse), 675 aa (fragment). | 16 . . . 675 | 607/660 | (91%) | |
| Q9CWK2 | NEURAL PRECURSOR CELL | 1 . . . 659 | 563/660 | (85%) | 0.0 |
| | EXPRESSED, | 1 . . . 660 | 606/660 | (91%) | |
| | DEVELOPMENTALLY DOWN- | | | | |
| | REGULATED GENE 1 - Mus | | | | |
| | musculus (Mouse), 660 aa. | | | | |
| Q9FI89 | SIMILARITY TO REGULATORY | 8 . . . 533 | 145/550 | (26%) | 4e−40 |
| | PROTEIN NEDD1 - Arabidopsis | 15 . . . 532 | 246/550 | (44%) | |
| | thaliana (Mouse-ear cress), 787 aa. | | | | |
| BAB75165 | WD-40 REPEAT PROTEIN - | 2 . . . 298 | 92/307 | (29%) | 2e−18 |
| | Anabaena sp. (strain PCC 7120), | 916 . . . 1208 | 147/307 | (46%) | |
| | 1526 aa. | | | | |

PFam analysis predicts that the NOV55a protein contains the domains shown in the Table 55F.

TABLE 55F
Domain Analysis of NOV55a

| | | Identities/ | | |
|---|---|---|---|---|
| | | Similarities | | |
| | NOV55a Match | for the Matched | | Expect |
| Pfam Domain | Region | Region | | Value |
| WD40: domain 1 of 7 | 28 . . . 61 | 6/37 | (16%) | 57 |
| | | 27/37 | (73%) | |
| WD40: domain 2 of 7 | 70 . . . 105 | 10/37 | (27%) | 0.062 |
| | | 27/37 | (73%) | |
| WD40: domain 3 of 7 | 111 . . . 147 | 9/37 | (24%) | 20 |
| | | 28/37 | (76%) | |
| WD40: domain 4 of 7 | 153 . . . 190 | 10/38 | (26%) | 3.4 |
| | | 29/38 | (76%) | |
| WD40: domain 5 of 7 | 197 . . . 234 | 7/38 | (18%) | 19 |
| | | 25/38 | (66%) | |
| WD40: domain 6 of 7 | 240 . . . 275 | 14/37 | (38%) | 3.1 |
| | | 28/37 | (76%) | |
| WD40: domain 7 of 7 | 282 . . . 316 | 8/37 | (22%) | 1.3e+03 |
| | | 26/37 | (70%) | |

### Example 56

The NOV56 clone was analyzed, and the nucleotide and predicted polypeptide sequences are shown in Table 56A.

TABLE 56A
NOV56 Sequence Analysis

| | SEQ ID NO:167 | 1771 bp | |
|---|---|---|---|
| NOV56a, | GACTGTTTCACCATGCAGTGGCTAATGAGGTTCCGGACCCTCTGGGGCATCCACAAAT | | |
| CG59439.01 DNA Sequence | | | |
| | CCTTCCACAACATCCACCCTGCCCCTTCACAGCTGCGCTCCCGGTCTTTATCAGAATT | | |
| | TGGAGCCCCAAGATGGAATGACTATGAAGTACCGGAGGAATTTAACTTTGCAAGTTAT | | |
| | GTACTGGACTACTGCGCTCAAAAGGAGAAGGACGGCAACAGAGGTCCAAATCCAGCTT | | |
| | TTTCGTGGGTGAATGGCCAAGGGGATGAAGTAAAGTGGAGCTTCAGAGAGATGGGAGA | | |
| | CCTAACCCGCCGTGTAGCCAACCTCTTCACACAGACCTGTGGCCTACAACAGGGACAC | | |
| | CATCTGGCCTTOATGCTGCCTCGAGTTCCTGAGTGGTGGCTCGTGGCTGTGGGCTGCA | | |
| | TGCGAACAGGGATCATCTTCATTCCTGCGACCATCCTGTTGAAGGCCAAAGACATTCT | | |
| | CTATCGACTACAGTTGTCTAAAGCCAAGGGCATTGTGACCATAGATGCCCTTGCCTCA | | |
| | GAGGTGGACTCCATAGCTTCTCAGTGCCCCTCTCTGAAAACCAAGCTCCTGGTGTCTG | | |
| | ATCACAGCCGTGAAGGGTGGCTGGACTTCCGATCGCTGGTTAAATCAGCATCCCCAGA | | |
| | ACACACCTGTGTTAAGTCAAAGACCTTGCACCCAATGGTCATCTTCTTCACCAGTGGG | | |
| | ACCACAGGCTTCCCCAAGATGGCAAAACACTCCCATGGGTTGGCCTTACAACCCTCCT | | |
| | TCCCAGGAAGTACGAAATTACGCAGCCTGAAGACATCTGATGTCTCCTGGTGCCTGTC | | |
| | GGACTCAGGATGGATTGTGGCTACCATTTGGACCCTGGTAGAACCATGGACAGCGGGT | | |
| | TGTACAGTCTTTATCCACCATCTGCCACAGTTTGACACCAAGGTCATCATACAGACAT | | |
| | TGTTGAAATACCCCATTAACCACTTTTGGGGGGTATCATCTATATATCGAATGATTCT | | |
| | GCAGCAGCATTTCACCACCATCAGGTTCCCTGCCCTGGAGCACTGCTATACTGGCCGG | | |
| | GAGGTCGTGTTGCCCAAGGATCAGGAGGAGTGGAAAAGACGGACCGGCCTTCTGCTCT | | |
| | ACGAGAACTATGGGCAGTCGGAAACGGGACTAATTTGTGCCACCTACTGGGGAATGAA | | |
| | GATCAAGCCGGGTTTCATGGGGAAGGCCACTCCACCCTACGACGTCCAGGTCATTGAT | | |
| | GACAAGGGCAGCATCCTGCCACCTAACACAGAAGGAAACATTGGCATCAGAATCAAAC | | |
| | CTGTCAGGCCTGTGAGCCTCTTCATGTGCTATGAGGGTGACCCAGAGAAGACAGCTAA | | |
| | AGTGGAATGTGGGGACTTCTACAACACTGGGGACAGAGGTAAGATGGATGAAGAGGGC | | |
| | TACATTTGTTTCCTGGGGAGGAGTCATGACATCATTAATGCCTCTGGGTATCGCATCG | | |
| | GGCCTGCAGAGGTTGAAAGCGCTTTGGTGGAGCACCCAGCGGTGGCGGAGTCAGCCGT | | |
| | GGTGGGCAGCCCAGACCCGATTCGAGGGGAGGTGGTGAAGGCCTTTATTGTCCTGACC | | |
| | CCACACTTCCTGTCCCATGACAAGGATCACCTGACCAAGCAACTGCAGCAGCATGTCA | | |
| | AGTCAGTGACAGCCCCATACAAGTACCCAAGGAAGGTGGAGTTTGTCTCAGAGCTGCC | | |
| | AAAAACCATCACTGGCAAGATTGAACCGAACGAACTTCCGAAAAACGAGACTGGTCAG | | |
| | ATGTAATCGGCAGTGAACTCAGAACGCACTG | | |
| | ORF Start: ATG at 13 | | ORF Stop: TAA at 1744 |
| | SEQ ID NO:168 | 577 aa | MW at 65272.6 kD |
| NOV56a, | MQWLMRFRTLWGIHKSFHNIHPAPSQLRCRSLSEFGAPRWNDYEVPEEFNFASYVLDY | | |
| CG59439-01 Protein Sequence | | | |
| | WAQKEKEGKRGPNPAFWWVNGQGDEVKWSFREMGDLTRRVANVFTQTCGLQQGDHLAL | | |
| | MLPRVPEWWLVAVGCMRTGIIFIPATILLKAKDILYRLQLSKAKGIVTIDALASEVDS | | |
| | IASQCPSLKTKLLVSDHSREGWLDFRSLVKSASPEHTCVKSKTLDPMVIFFTSGTTGF | | |
| | PKMAKHSHGLALQPSFPGSRKLRSLKTSDVSWCLSDSGWIVATTWTLVEPWTAGCTVF | | |
| | IHHLPQFDTKVIIQTLLKYPINHFWGVSSTYRMILQQDFTSIRFPALEHCYTGGEVVL | | |
| | PKDQEEWKRRTGLLLYENYGQSETGLICATYWGMKIKPGFMGKATPPYDVQVIDDKGS | | |
| | ILPPNTEGNTGIRIKPVRPVSLFMCYEGDPEKTAKVECGDFYNTGDRGKMDEEGYICF | | |
| | LGRSDDIINASGYRIGPAEVESALVEHPAVAESAVVGSPDPIRGEVVKAFIVLTPQFL | | |
| | SHDKDQLTKELQQHVKSVTAPYKYPRKVEFVSELPKTITGKIERKELRKKETGQM | | |
| | SEQ ID NO:169 | 1659 bp | |
| NOV56b, | GTTTCACCATGCAGTCGCTAATCACGTTCCGGACCCTCTGGGGCATCCACAAATCCTT | | |
| CG59539-02 DNA Sequence | | | |
| | CCACAACATCCACCCTGCCCCTTCACAGCTGCGCTGCCGGTCTTTATCAGAATTTGGA | | |
| | GCCCCAACATGGAATGACTATGAAGTACCGGAGGAATTTAACTTTGCAACTTATGTAC | | |
| | TGGACTACTGGGCTCAAAAGGAGAAGGAGGGCAAGAGAGGTCCAAATCCAGCTTTTTG | | |
| | GTGCGTGAATCCCCAAGGCGATGAAGTAAAGTGCAGCTTCAGAGAGATGGCAGACCTA | | |
| | ACCCGCCGTGTAGCCAACGTCTTCACACAGACCTGTGCCCTACAACAGGCAGACCATC | | |
| | TGGCCTTGATGCTGCCTCGAGTTCCTGAGTGGTGGCTGGTGGCTGTGGGCTGCATGCG | | |
| | AACAGGGATCATCTTCATTCCTGCGACCATCCTGTTGAAGGCCAAAGACATTCTCTAT | | |
| | CGACTACAGTTGTCTAAAGCCAAGGCCATTGTGACCATAGATCCCCTTGCCTCAGAGG | | |
| | TGGACTCCATAGCTTCTCAGTGCCCCTCTCTGAAAACCAAGCTCCTGGTGTCTGATCA | | |
| | CAGCCGVCAAGGGTGGCTGGACTTCCGATCGCTGGTTAAATCAGCATCCCCAGAACAC | | |
| | ACCTGTGTTAACTCAAAGACCTTGGACCCAATGGTCATCTTCTTCACCACTGGGACCA | | |
| | CAGGCTTCCCCAAGATGGCAAAACACTCCCATGGGTTCGCCTTACAACCCTCCTTCCC | | |
| | AGGAAGTAGGAAATTACGGAGCCTCAAAACATCTGATGTCTCCTGGTGCCTGTCGGAC | | |
| | TCACGATGCATTGTGGCTACCATTTGGACCCTGGTAGAACCATGGACAGCGGGTTGTA | | |
| | CAGTCTTTATCCACCATCTGCCACAGTTTGACACCAAGGTCATCATACAGACATTGTT | | |
| | GAAATACCCCATTAACCACTTTTGGGGGGTATCATCTATATATCGAATGATTCTGCAG | | |
| | CAGGATTTCACCAGCATCAGGTTCCCTGCCCTGGAGCACTGCTATACTGGCGGGGAGG | | |
| | TCGTGTTGCCCAAGGATCACGAGGAGTCGAAAAGACGGACGGGCCTTCTGCTCTACGA | | |
| | GAACTATCCCCACTCGGAAACGGGACTAATTTGTGCCACCTACTCGGGAATGAAGATC | | |
| | AAGCCGCGTTTCATGGGGAAGGCCACTCCACCCTACGACCTCCAGGGTGACCCAGAGA | | |
| | AGACAGCTAAAGTGGAATGTGGCGACTTCTACAACACTGGGGACAGAGGAAAGATGGA | | |
| | TGAAGAGGGCTACATTTGTTTCCTGGGGAGGAGTGATCACATCATTAATGCCTCTGGG | | |
| | TATCGCATCGGGCCTGCAGAGGTTGAAAGTGCTTTGGTGGAGCACCCAGCGGTCGCGG | | |
| | AGTCAGCCGTGGTGGGCAGCCCAGACCCGATTCGAGGGGAGGTGGTGAAGGCCTTTAT | | |
| | TGTCCTGACCCCACAGTTCCTGTCCCATGACAAGGATCAGCTGACCAAGGAACTGCAG | | |
| | CAGCATCTCAAGTCAGTGACAGCCCCATACAAGTACCCAAGGAAAGTGGAGTTTGTCT | | |
| | CAGAGCTGCCAAAAACCATCACTGGCAAGATTGAACGGAAGGAACTTCGGAAAAAGGA | | |
| | CACTGGTCAGATGTAATCGGCAGTGAACTCAGAAC | | |
| | ORF Start: ATG at 9 | | ORF Stop: TAA at 1638 |
| | SEQ ID NO:170 | 543 aa | MW at 61518.2 kD |

| | |
|---|---|
| NOV56b, | MQWLMRFRTLWGIHHSFHNIHPARSQLRCRSLSEFGAPRWNDYEVPEEFNFASYVLDY |
| CG59439-02 Protein Sequence | |
| | WAQKEKEGKRGPNPAFWWVNGQGDEVKWSFREMGDLTRRVANVFTQTCGLQQGDHLAL |
| | MLPRVPEWWLVAVGCMRTGIIFIPATILLKAKDILYRLQLSKAKGIVTIDALASEVDS |
| | IASQCPSLKTKLLVSDHSREGWLDFRSLVKSASPEHTCVKSKTLDPMVIFFTSGTTCF |
| | PKMAKHSHGLALQPSFPGSRKLRSLKTSDVSWCLSDSGWTVATTWTLVEPWTAGCTVF |
| | IHHLPQFDTKVIIQTLLKYPINHFWGVSSIYRMILQQDFTSIRFPALEHCYTGGEVVL |
| | PKDQEEWKRRTGLLLYENYGQSETGLICATYWGMKIKPGFMGKATPPYDVQGDPEKTA |
| | KVECGDFYNTGDRGKMDEEGYICFLGRSDDIINASGYRIGPAEVESALVEHPAVAESA |
| | VVGSPDPTRGEVVKAFIVLTPQFLSHDKDQLTKELQQHVKSVTAPYKYPRKVEFVSEL |
| | PKTTTGKIERKELRKKETCQM |

Sequence comparison of the above protein sequences yields the following sequence relationships shown in Table 56B.

TABLE 56B
Comparison of NOV56a against NOV56b.

| | | Identities/ |
|---|---|---|
| | NOV56a Residues/ | Similarities for the |
| Protein Sequence | Match Residues | Matched Region |
| NOV56b | 1 . . . 577 | 543/577 (94%) |
| | 1 . . . 543 | 543/577 (94%) |

Further analysis of the NOV56a protein yielded the following properties shown in Table 56C.

TABLE 56C
Protein Sequence Properties NOV56a

| | |
|---|---|
| PSort | 0.6400 probability located in microbody (peroxisome); |
| analysis: | 0.4712 probability located in mitochondrial matrix space; |
| | 0.1737 probability located in mitochondrial inner membrane; |
| | 0.1737 probability located in mitochondrial |
| | intermembrane space |
| SignalP | Like1y cleavage site between residues 23 and 24 |
| analysis: | |

A search of the NOV56a protein against the Geneseq database, a proprietary database that contains sequences published in patents and patent publication, yielded several homologous proteins shown in Table 56D.

TABLE 56D
Geneseq Results for NOV56a

| | | NOV56a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAB43245 | Human ORFX ORF3009 | 46 . . . 573 | 309/529 | (58%) | 0.0 |
| | polypeptide sequence SEQ ID | 1 . . . 527 | 402/529 | (75%) | |
| | NO:6018 - Homo sapiens, 537 aa. | | | | |
| | [WO200058473-A2, 5 OCT | | | | |
| | 2000] | | | | |
| AAM80008 | Human protein SEQ ID NO 3654 - | 331 . . . 577 | 247/279 | (88%) | e−140 |
| | Homo sapiens, 302 aa. | 24 . . . 302 | 247/279 | (88%) | |
| | [WO200157190-A2, 9 AUG | | | | |
| | 2001] | | | | |
| AAM80007 | Human protein SEQ ID NO 3653 - | 331 . . . 577 | 247/279 | (88%) | e−140 |
| | Homo sapiens, 302 aa. | 24 . . . 302 | 247/279 | (88%) | |
| | [WO200157190-A2, 9 AUG | | | | |
| | 2001] | | | | |
| AAM41894 | Human polypeptide SEQ ID NO | 257 . . . 573 | 193/317 | (60%) | e−116 |
| | 6825 - Homo sapiens, 390 aa. | 7 . . . 323 | 246/317 | (76%) | |
| | [WO200153312-A1, 26 JUL 2001] | | | | |
| AAM79024 | Human protein SEQ ID NO 1686 - | 382 . . . 577 | 196/196 | (100%) | e−112 |
| | Homo sapiens, 196 aa. | 1 . . . 196 | 196/196 | (100%) | |
| | [WO200157190-A2, 9 AUG | | | | |
| | 2001] | | | | |

In a BLAST search of public sequence databases, the NOV56a protein was found to have homology to the proteins shown in the BLASTP data in Table 56E.

TABLE 56E
Public BLASTP Results for NOV56a

| | | NOV56a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96A20 | MIDDLE-CHAIN ACYL-COA | 1 . . . 577 | 576/577 (99%) | 0.0 |
| | SYNTHETASE1 (MEDIUM- | 1 . . . 577 | 576/577 (99%) | |
| | CHAIN ACYL-COA | | | |
| | SYNTHETASE) - Homo sapiens | | | |
| | (Human), 577 aa. | | | |
| Q9TVB5 | XENOBIOTIC/MEDIUM-CHAIN | 1 . . . 576 | 439/576 (76%) | 0.0 |
| | FATTY ACID:COA LIGASE | 1 . . . 576 | 486/576 (84%) | |
| | FORM XL-III PRECURSOR - Bos | | | |
| | taurus (Bovine), 577 aa. | | | |
| Q9BEA2 | LIPOATE-ACTIVATING | 1 . . . 576 | 438/576 (76%) | 0.0 |
| | ENZYME PRECURSOR - Bos | 1 . . . 576 | 485/576 (84%) | |
| | taurus (Bovine), 577 aa. | | | |
| Q91VA0 | MEDIUM-CHAIN ACYL-COA | 1 . . . 577 | 406/577 (70%) | 0.0 |
| | SYNTHETASE (EC 6.2.1.2) | 1 . . . 573 | 472/577 (81%) | |
| | (HYPOTHETICAL 64.8 KDA | | | |
| | PROTEIN) - Mus musculus | | | |
| | (Mouse), 573 aa. | | | |
| O70490 | KIDNEY-SPECIFIC PROTEIN - | 1 . . . 573 | 315/580 (54%) | 0.0 |
| | Rattus norvegicus (Rat), 572 aa. | 1 . . . 567 | 417/580 (71%) | |

PFam analysis predicts that the NOV56a protein contains the domains shown in the Table 56F.

TABLE 56F
Domain Analysis of NOV56a

| | | Identities/ | |
|---|---|---|---|
| | | Similarities | |
| | NOV56a Match | for the Matched | Expect |
| Pfam Domain | Region | Region | Value |
| AMP-binding: | 87 . . . 499 | 106/425 (25%) | 2.5e−96 |
| domain 1 of 1 | | 299/425 (70%) | |

### Example 57

The NOV57 clone was analyzed, and the nucleotide and predicted polypeptide sequences are shown in Table 57A.

TABLE 57A
NOV57 Sequence Analysis

| | SEQ ID NO:171 | 2501 bp | |
|---|---|---|---|
| NOV57A, | ACACCATGACCACCCTTCATGATAAGTTCCTGGCGGAGAAACTCCAGTACTACTATAG | | |
| CG59354-01 DNA Sequence | | | |
| | CAGCAGTGAGGATGAGGACAGTGACCACCAGGACAAGGACCGAGGCAGATGTGCCCCA | | |
| | GCCAGCAGTTCTGTGCCTGCAGAGGCTGACCTGGCAGCCCAAGGCATCTCAGTTAACA | | |
| | CAATGACTCTGAAGGAGTTTGCCATAATGAATGAGGACCAAGATGATGAAGAGTTTCT | | |
| | GCAGCAGTACCCGAAGCAGCGAATGGAAGAGATGCGGCAGCAGCTTCACAAGGGGCCC | | |
| | CAATTCAAGCAAGTTTTTGAGATCTCCAGTGGAGAACGGTTTTTACACATCATTGATA | | |
| | AAGAACAGAAAAGCATTGTCATCATCGTTCATATTTATGAGGATGGCATTCCAGGGAC | | |
| | CGAACCCATGAATGGTTGCATGATCTGCCTTGCCGCAGAGTACCCAGCTGTCAAGTTC | | |
| | TGCAAGGTGAAGACCTCACTTATTGCCGCCAGCAGTCAGTTCACCAGGAATGCCCTTC | | |
| | CTGCCCTGCTGATCTATAAGGGGGGTGAATTGATCGGCAATTTTGTTCGTGTTACTGA | | |
| | CCAGCTGGGGGATGATTTCTTTGCTGTGCACCTTGAAGCTTTTCTCCAGGAATTTGGA | | |
| | TTACTCCCAGAAAAGGAAGTCTTGGTGCTGACATCTGTGCGTAACTCTGCCACGTGTC | | |
| | ACAGTGAGGATAGCGACCTGGAAATAGATTGAACTGATAGTCTAGTTGCATAGATTTC | | |
| | TCATTGTTTGGGTTGGAATACACGTCATTCTTTATTTTTGTTCCTTTGTCTTCTGGCT | | |
| | TTTCAGCTGTTCTTTGTAGTCCCTTTTATTATGCATAAAATAAAGAAATTCTTAGATT | | |
| | AAATCAGAATGCTGAATAACCTTGTAGCTAGCAATAAGGTGACTTACAATTGTATAAA | | |
| | CAGGAAGCCAGGCTTTTGAACTGTTTACTTAAGATTCTGTGGTGTGACATCTCTGTTA | | |
| | TTGTTTCCAGTCAATATTTACAAAGCATCCTAAAGACAGGGTCTTGGAAATTGTCTTC | | |
| | AGATGATCTTAGAGGTCTCTGCCAAGTCTGAGAGTATAATTCTGTAGGTATTGTGTTA | | |
| | TTTGCAACGTAAATAGTGCATTTTCTTAATCAAATGATTGTAAATTATATTTACTTGT | | |
| | AATCAGTTCCATAGCTTTAGACGGTGGTTAGATTTTTTTTTTCCCCACCAGGGTCTTG | | |
| | TTTAAAGGGGTCAGCCACCGCACCCAGTCCTGAGGGGTGGCCTCTGCTGCTGGATTTC | | |
| | ATGTCTTCCTCCAGCATGACTAACTCTGGAACAGCAGCAAGCGTTGATGCTTACTGAC | | |
| | CTGGTGATGTTAGAAGACAAGTAGTTTATGCATTTAAACATTAGAGCTGCAGTGGGGC | | |
| | TGGAAATCTTTGTAAAGGAAGTTCTTTCAGTAAGATGCCCCTGCTTGTCTTTGTCTCT | | |
| | TTTTTGTTTAACAAGGTAACTTTTTGTTTAACAAGGTAACTTTTTGTTTAACCTAGAT | | |
| | TTTTTTTAAAACTTTTTTTTTTTTTCATATTGGAAAAGTAATTCATATTCAGTAGAGG | | |
| | AAAACTGACCAAAACAGAAGCAAAAATAAGAAAATTAAAATAATCTCTAATCCTACTA | | |
| | CCTAGAATAAAACACTATTAATATTTTGGTCTGTTTCCTGCCAAGGTGTTTTCTGTGT | | |
| | ATACATGGATATTTTGTTTGTTTTTAAACAAAACGATGGGATCATTCTGAACATACTG | | |
| | TTCTATAGTATGGTCAGCTAATAATATATCAGACCTTTTTTTTATATTATTAAATATT | | |
| | CTACAACTTTTTAAAAATGTCTATTAATATTCCATCGTATAGATGTGATATAATTTGC | | |
| | TTGATGGTTGTCTCTTAAAAAGAAAGATAGCAAATACTTTTTTTAAATTACAAAAGTG | | |
| | ATAGATGTTCATTGTAGAAAATGTAATAAACACTGTTAAGACTTAAAAGCCATATAAT | | |
| | TCCACCAACCAAAATTAATCCCTTTTGTCATATTTCTAGTCATTTTTATAGCCTTTTT | | |
| | TTTCTATGTATTTATAATAATTATCATTTGCGTTTTTTTCCTTTTTTTAACTTTAAAA | | |
| | ATGTATATTCTAGGGTCAGGGGAAATGTAATCTGGAATTAAATATTAGCCTTAAAATT | | |
| | CACAATTTTGATTTTCCTGGCTTTTCAGGAATTGACTAACTGTAAAAGAGTCTTGAAA | | |
| | GTATTTAGTCAACAAACAGAGTGCATTTTTTTTTTTTTGACTAAGAAAGCTCGTTGTA | | |
| | GTAGAAAGGGTGGAATGTATTGAAAATTATTAGAAGCAGGGAAGTATTGTTAGTCTAG | | |
| | CTTATTTCCTTTCAGTCTTTTTTCAATATTTTTATAAACATTGAGTACTTACTGAATT | | |
| | TAGTTCTGTGCTCTTCCTTATTTAGTGTTGTATCATAAATACTTTGATGTTTCAAACA | | |
| | TTCTAATAAATAATTTTCAGTGGCTTCATAATAAAAAAAAAAAAAAAAAAAAAAAAAA | | |
| | AAAAAAA | | |
| | ORF Start: ATG at 6 | | ORF Stop: TGA at 726 |
| | SEQ ID NO:172 | 240 aa | MW at 26866.9 kD |
| NOV57a, | MTTLDDKLLGEKLQYYYSSSEDEDSDHEDKDRGRCAPASSSVPAEAELAGEGISVNTM | | |
| CG59354-01 Protein Sequence | | | |
| | TLKEFAIMNEDQDDEEFLQQYRKQRMEEMRQQLHKGPQFKQVFEISSGEGFLDMIDKE | | |
| | QKSTVTMVHIYEDGIPGTEANNGCMTCLAAEYPAVKFCKVKSSVIGASSQETRNALPA | | |
| | LLIYKGGELIGNFVRVTDQLGDDFFAVDLEAFLQEFGLLPEKEVLVLTSVRNSATCHS | | |
| | EDSDLEID | | |
| | SEQ ID NO:173 | 893 bp | |
| NOV57b, | CACCATGACCACCCTGTATGATAAGTTGCTGGGCCAGAAACTGCAGTACTACTATAGC | | |
| CG59354-02 DNA Sequence | | | |
| | AGCAGTGAAGATGAGGACAGTGACCACGAGGACAAGGACCGAGCCATCTCAGTTAACA | | |
| | CAGGCCCAAAAGGTGTGATCAATGACTAGCGCCGCTTCAAGCAGTTGGAGACACACCA | | |
| | GAGGGAGGAGCAGTGCCGGGAGATGGAAAGGCTGATCAAGAAGCTGTCAATGACTTGC | | |
| | AGGTCCCATCTGGATGAAGAGGAGGAGCAACAGAAACAGAAAGACCTCCAGGAGAAGA | | |
| | TCAGTGGGAACATGACTCTGAAGGAGTTTGCCATAATGAATGAGGACCAACATGATGA | | |
| | AGAGTTTCTGCAGCAGTACCGGAAGCAGCGAATCGAAGAGATGCGGCAGCAGCTTCAC | | |
| | AAGCGCCCCCAATTCAAGCAGGTTTTTGAOATCTCCAGTGGAGAAGGGTTTTTAGACA | | |
| | TGATTGATAAAGAACAGAAAAGCATTGTCATCATGGTTCATATTTATGAGGATGGCAT | | |
| | CAGGGACCGAAGCCATGAATGGTTGCATGATCCCCCTTGCAAGGGGCGTGAATTGATC | | |
| | GGCAATTTTGTTCGTGTTACTGACCAGCTGGGOGATGATTTCTTTGCTGTCGACCTTG | | |
| | AAGCTTTTCTCCAGGAATTTGGATTACTCCCAGAAAAGGAAGTCTTGGTCCTGACATC | | |
| | TGTGCGTAACTCTGCCACGTGTCACAGTGAGGATAGCGACCTGGAAATAGATTGAACT | | |
| | GATAGTCTAGTTGCATATAGATTTCTCATTGTTTGGGTTGGAATACACCATTGTTTAT | | |
| | TTTTQTTCCTTTGTCTTCTGGCTTTTCAGCTGTTCTTTGTAGTCCCTTTTATTATGCA | | |
| | TAAAATAAAAAAATTCTTAGATT | | |
| | ORF Start: ATG at 5 | | ORF Stop: TGA at 749 |
| | SEQ ID NO:174 | 248 aa | MW at 29227.4 kD |
| NOV57b, | MTTLYDKLLGEKLQYYYSSSEDEDSDHEDKDRGISVNTGPKGVINDWRRKFQLETEQR | | |
| CG59354-02 Protein Sequence | | | |
| | EEQCREMERLIKKLSMTCRSHLDEEEEQQKQKDLQEKTSGKMTLKEFAIMTEDQDDEE | | |
| | FLQQYRKQRNEEMRQQLHKGPQFKQVFEISSGEGFLDMIDKEQKSIVIMVHIYEDGIR | | |
| | DRSHEWLHDPPCKGGELIGNFVRVTDQLGDDFFAVDLEAFLQEFGLLPEKEVLVLTSV | | |
| | RNSATCHSEDSDLEID | | |
| | SEQ ID NO:175 | 891 bp | |
| NOV57c, | CACCATGACCACCCTGTATGATAAGTTGCTGGGGGAGAAACTGCAGTACTACTATAGC | | |
| CG59354-02 DNA Sequence | | | |
| | AGCAGTGAAGATGAGGACAGTGACCACGAGGACAAGGACCGAGGCATCTCAGTTAACA | | |
| | CAGGCCCAAAAGGTGTGATCAATGACTGGCGCCGCTTCAAGCAGTTGGAGACAGAGCA | | |
| | GAGGGAGGAGCAGTGCCGGGAGATGGAAAGGCTCATCAAGAAGCTGTCAATGACTTGC | | |
| | AGGTCCCATCTGGATGAAGAGGAGGAGCAACAGAAACAGAAAGACCTCCAGGAGAAGA | | |
| | TCAGTGGGAAGATGACTCTGAAGGAGTTTGCCATAATGAATGAGGACCAAGATGATGA | | |
| | AGAGTTTCTGCAGCAGTACCGGAAGCAGCGAATGGAAGAGATGCGGCAGCAGCTTCAC | | |
| | AAGGGGCCCCAATTCAAGCAGGTTTTTCAGATCTCCAGTGGAGAAGGGTTTTTAGACA | | |
| | TGATTGATAAAGAACAGAAAAGCATTGTCATCATGGTTCATATTTATGAGCATGGCAT | | |
| | TCCAGGGACCGAAGCCATGAATGGTTGCATGATCCGCCTTGCCGCAGAGTACCCACCT | | |
| | GTCAAGTTCTGCAAGGTGAAGAGCTCAGTTATTGGCGCCAGCAGTCAGTTCACCAGGA | | |
| | ATGCCCTTCCTGCCCTGCTGATCTATAAGGGGGGTGAATTGATCGGCAATTTTGTTCG | | |
| | TGTTACTGACCAGCTGGGGGATGATTTCTTTGCTGTGGACCTTGAAGCTTTTCTCCAG | | |
| | GAATTTGGATTACTCCCAGAAAAGGAAGTCTTGGTGCTGACATCTGTGCGTAACTCTG | | |
| | CCACGTGTCACAGTGAGGATAGCGACCTGGAAATAGATTGAACTGATAGTCTAGTTGC | | |
| | ATAGATTTCTCATTGTTTGGG | | |
| | ORF Start: ATG at 5 | | ORF Stop: TGA at 851 |
| | SEQ ID NO:176 | 272 aa | MW at 32598.5 kD |

| | |
|---|---|
| NOV57c, | MTTLYDKLLGEKLQYYYSSSEDEDSDHEDRDRGISVNTGPKGVINDWRRFKQLETEQR |
| CG59354-03 Protein Sequence | |
| | EEQCREMERLIKKLSMTCRSHLDEEEEQQKQKDLQEKISGKMTLKEFAIMNEDQDDEE |
| | FLQQYRKQRMEEMRQQLEKGPQFKQVFETSSGEGFLDMIDKEQKSIVIMVHIYEDGIP |
| | GTEAMNGCMIRLAAEYPAVKFCKVKSSVICASSQETRNALPALLIYKGGELIGNFVRV |
| | TDQLGDDEFAVDLEAFLQEFGLLPEKEVLVLTSVRNSATCHSEDSDLEID |

Sequence comparison of the above protein sequences yields the following sequence relationships shown in Table 57B.

TABLE 57B
Comparison of NOV57a against NOV57b through NOV57c.

| | | Identities/ |
|---|---|---|
| | NOV57a Residues/ | Similarities for the |
| Protein Sequence | Match Residues | Matched Region |
| NOV57b | 58 . . . 240 | 138/183 (75%) |
| | 100 . . . 248 | 140/183 (76%) |
| NOV57c | 58 . . . 240 | 182/183 (99%) |
| | 100 . . . 282 | 182/183 (99%) |

Further analysis of the NOV57a protein yielded the following properties shown in Table 57C.

TABLE 57C
Protein Sequence Properties NOV57a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV57a protein against the Geneseq database, a proprietary database that contains sequences published in patents and patent publication, yielded several homologous proteins shown in Table 57D.

TABLE 57D
Geneseq Results for NOV57a

| | | NOV57a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAE03537 | Human secreted | 1 . . . 240 | 226/301 (75%) | e−117 |
| | protein variant, | 1 . . . 301 | 230/301 (76%) | |
| | SEQ ID NO: | | | |
| | 228—Homo | | | |
| | sapiens, 301 aa. | | | |
| | [WO200132675- | | | |
| | A1, May 10, | | | |
| | 2001] | | | |
| AAY99657 | Human GTPase | 1 . . . 240 | 226/301 (75%) | e−117 |
| | associated | 1 . . . 301 | 230/301 (76%) | |
| | protein-8— | | | |
| | Homo sapiens, | | | |
| | 301 aa. | | | |
| | [WO200031263- | | | |
| | A2, Jun. 2, 2000] | | | |
| AAE02004 | Fruitfly viral | 55 . . . 214 | 52/161 (32%) | 3e−14 |
| | IAP-associated | 59 . . . 213 | 86/161 (53%) | |
| | factor (VIAF)— | | | |
| | Drosophila | | | |
| | melanogaster, | | | |
| | 240 aa. | | | |
| | [WO200134798- | | | |
| | A1, May 17, | | | |
| | 2001] | | | |
| AAE02003 | Zebrafish viral | 21 . . . 236 | 69/241 (28%) | 5e−13 |
| | IAP-associated | 2 . . . 237 | 117/241 (47%) | |
| | factor (VIAF)— | | | |
| | Brachydanio | | | |
| | rerio, 239 aa. | | | |
| | [WO200134798- | | | |
| | A1, May 17, | | | |
| | 2001] | | | |
| AAE02002 | Mouse viral | 58 . . . 240 | 59/195 (30%) | 4e−12 |
| | IAP-associated | 52 . . . 240 | 99/195 (50%) | |
| | factor (VIAF)— | | | |
| | Mus musculus, | | | |
| | 240 aa. | | | |
| | [WO200134798- | | | |
| | A1, May 17, | | | |
| | 2001] | | | |

In a BLAST search of public sequence databases, the NOV57a protein was found to have homology to the proteins shown in the BLASTP data in Table 57E.

TABLE 57E
Public BLASTP Results for NOV57a

| | | NOV57a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q96AF1 | HYPOTHETICAL | 1 . . . 240 | 226/301 (75%) | e−117 |
| | 34.3 KDA | 1 . . . 301 | 230/301 (76%) | |
| | PROTEIN—Homo | | | |
| | sapiens (Human), | | | |
| | 301 aa. | | | |
| Q13371 | Phosducin-like | 1 . . . 240 | 225/301 (74%) | e−116 |
| | protein (PHLP)— | 1 . . . 301 | 230/301 (75%) | |
| | Homo sapiens | | | |
| | (Human), 301 aa. | | | |
| T17321 | hypothetical protein | 1 . . . 240 | 225/301 (74%) | e−116 |
| | DKFZp564M1863.1— | 1 . . . 301 | 230/301 (75%) | |
| | human, 301 aa. | | | |
| Q923E8 | RIKEN CDNA | 1 . . . 240 | 210/301 (69%) | e−109 |
| | 1200011E13 GENE— | 1 . . . 301 | 223/301 (73%) | |
| | Mus musculus | | | |
| | (Mouse), 301 aa. | | | |
| Q63737 | Phosducin-like protein | 1 . . . 240 | 210/301 (69%) | e−108 |
| | (PHLP)—Rattus | 1 . . . 301 | 223/301 (73%) | |
| | norvegicus (Rat), | | | |
| | 301 aa. | | | |

PFam analysis predicts that the NOV57a protein contains the domains shown in the Table 57F.

TABLE 57F
Domain Analysis of NOV57a

| | | Identities/ | |
|---|---|---|---|
| | NOV57a | Similarities for | Expect |
| Pfam Domain | Match Region | the Matched Region | Value |
| Phosducin: domain | 35 . . . 57 | 14/23 (61%) | 8.7e−08 |
| 1 of 2 | | 21/23 (91%) | |
| Phosducin: domain | 58 . . . 240 | 133/183 (73%) | 9.7e−148 |
| 2 of 2 | | 174/183 (95%) | |

### Example 58

The NOV58 clone was analyzed, and the nucleotide and predicted polypeptide sequences are shown in Table 58A.

TABLE 58A
NOV58 Sequence Analysis

| | SEQ ID NO:177 | 756 bp | |
|---|---|---|---|
| NOV58a, | GGATCCCAATGAAGATACAGAATGGAATGACATTTTAAGAGATTTCCGCATTCTTCCT | | |
| CG59319-01 DNA Sequence | | | |
| | CCTAAAGAAGAGTCAAAAGATGAAATTGAAGAAATGGTTTTACGTTTACAGAAAGAAG | | |
| | CAATGGTGAAACCATTTGAAAAGATGACTCTTGCACAGCTAAAGGAAGCTGAAGATGA | | |
| | ATTCGATGAAGAAGATATGCAGGCTGTTGAAACATATAGAAAGAAGCGGTTACAGGAA | | |
| | TGGAAAGCTCTTAAGAAAAAACAAAAATTTGGACAATTAACAGAAATTTCTGGAAATC | | |
| | AGTATGTGAATGAAGTCACAAATGCAGAAGAAGATGTGTGGGTTATAATTCATCTATA | | |
| | CAGATCAAGCATCCCAATGTGTTTGTTGGTTAACCAGCATCTTAGTCTTCTAGCAAGA | | |
| | AAGTTTCCAGAAACTAAATTTGTTAAAGCCATCGTGAATAGCTGTATTCAACACTACC | | |
| | ATGACAATTGTTTACCAACAATTTTTGTGTATAAAAATGGTCAGATAGAACCCAAATT | | |
| | CATTGGAATTATAGAATGTGCAOGGATAAATCTCAAGCTGOAAGAACTTGAATGGAAG | | |
| | CTAGCACAACTTGGAGCAATACAGACTCATTTGCAACAAAACCCCACAAAAGACATGG | | |
| | TAGATATGATGGTATCTTCAATTAGAAACACTTCTATTCATGATGACAGTGATAGCTC | | |
| | CAACAGTGATAATGATACCAAATAGAGAGAAATATTCAATAAATAGCTTTTAGTAAAA | | |
| | AA | | |
| | ORF Start: GAT at 2 | | ORF Stop: TAG at 719 |
| | SEQ ID NO:178 | 239 aa | MW at 27811.3 kD |
| NOV58a, | DPNEDTEWNDILRDFGILPPKEESKDEIEEMVLRLQKEAMVKPFEKMTLAQLKEAEDE | | |
| CG59319-01 Protein Sequence | | | |
| | FDEEDMQAVETYRKKRLQEWKALKKKQKFGELREISGHQYVNEVTNAEEDVWVIIHLY | | |
| | RSSIPMCLLVNQHLSLLARKFPETKFVKATVNSCIQHYHDNCLPTTFVYKNGQIEAKF | | |
| | IGIIECGGINLKLEELEWKLAEVGATQTDLEENPRKDMVDMMVSSIRNTSIHDDSDSS | | |
| | NSDNDTK | | |
| | SEQ ID NO:179 | 745 bp | |
| NOV58b, | GGATCCCAATGAAGATACAGAATGGATCCCAATGAAGATACAGAATGGAATGACATTT | | |
| CG59319-02 DNA Sequence | | | |
| | TAAGACATTTCGGCATTCTTCCTCCTAAAGAAGAGTCAAAAGATGAAATTGAAGAAAT | | |
| | GGTTTTACGTTTACAGAAAGAAGCAATGGTGAAACCATTTGAAAAGATGACTCTTGCA | | |
| | CAGCTAAAGGAAGCTGAAGATGAATTTGATGAAGAAGATATGCAGGCTGTTGAAACAT | | |
| | ATAGAAAGAAGCGGTTACAGGAATGGAAACCTCTTAACAAAAAACAAAAATTTGGAGA | | |
| | ATTAAGAGAAATTTCTCGAAATCAGTATGTGAATGAAGTCACAAATGCAOAAGAAGAT | | |
| | GTGTCGGTTATAATTCATCTATACAGATCAAGCATCCCAATGTGTTTGTTGGTTAACC | | |
| | AGCATCTTAGTCTTCTAGCAAGAAAGTTTCCAGAAACTAAATTTGTTAAAGCCATCGT | | |
| | GAATAGCTGTATTCAACACTACCATGACAATTGTTTACCAACAATTTTTGTGTATAAA | | |
| | AATGCTCACATAGAAGCCAAATTCATTGGAATTATAGAATCTGGAGGGATAAATCTCA | | |
| | AGCTGGAAGAACTTGAATGGAAGCTAGCAGAAGTTGGAGCAATACAGACTGATTTGGA | | |
| | AGAAAACCCCAGAAAACACATCGTAGATATCATGCTATCTTCAATTAGAAACACTTCT | | |
| | ATCCATGATGACAGTGATAGCTCCAACAGTGATAATGATACCAAATAGA | | |
| | ORF Start: ATG at 22 | | ORF Stop: TAG at 742 |
| | SEQ ID NO:180 | 240 aa | MW at 27942.5 kD |

| | |
|---|---|
| NOV58b, | MDPNEDTEWNDILRDFCTLPPKEESKDEIEEMVLRLQKEANVKPBERMTLAQLKEAED |
| CG59319-02 Protein Sequence | |
| | EFDEEDNQAVETYRKKRLQEWKALKKKQKFGELRETSGNQYVNEVTNAEEDVWVIIHL |
| | YRSSIPMCLLVNQHLSLLARKFPETKFVKAIVNSCIQHYHDNCLPTIFVYKNGQIAEK |
| | FIGIIECGGINLKLEELEWKLAEVGAIQTDLEENPRKDMVDMMVSSIRNTSIHDDSDS |
| | SNSDNDTK |

Sequence comparison of the above protein sequences yields the following sequence relationships shown in Table 58B.

TABLE 58B
Comparison of NOV58a against NOV58b.

| Protein | NOV58a Residues/ | Identities/Similarities |
|---|---|---|
| Sequence | Match Residues | for the Matched Region |
| NOV58b | 1 . . . 239 | 216/239 (90%) |
| | 2 . . . 240 | 216/239 (90%) |

Further analysis of the NOV58a protein yielded the following properties shown in Table 58C.

TABLE 58C
Protein Sequence Properties NOV58a

| | |
|---|---|
| PSort | 0.8800 probability located in nucleus; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV58a protein against the Geneseq database, a proprietary database that contains sequences published in patents and patent publication, yielded several homologous proteins shown in Table 58D.

TABLE 58D
Geneseq Results for NOV58a

| | | NOV58a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAE02003 | Zebrafish viral | 1 . . . 237 | 133/238 (55%) | 3e−75 |
| | IAP-associated | 3 . . . 239 | 181/238 (75%) | |
| | factor (VIAF)— | | | |
| | Brachydanio rerio, | | | |
| | 239 aa. | | | |
| | [WO200134798-A1, | | | |
| | May 17, 2001] | | | |
| AAU27979 | Mouse contig | 1 . . . 231 | 137/234 (58%) | 2e−74 |
| | polypeptide | 7 . . . 240 | 176/234 (74%) | |
| | sequence #132— | | | |
| | Mus musculus, | | | |
| | 243 aa. | | | |
| | [WO200164834-A2, | | | |
| | Sep. 7, 2001] | | | |
| AAU27807 | Human full-length | 1 . . . 231 | 137/234 (58%) | 2e−74 |
| | polypeptide | 3 . . . 236 | 176/234 (74%) | |
| | sequence #132— | | | |
| | Mus musculus, | | | |
| | 239 aa. | | | |
| | [WO200164834-A2, | | | |
| | Sep. 7, 2001] | | | |
| AAE02001 | Human viral IAP- | 1 . . . 231 | 137/234 (58%) | 2e−74 |
| | associated factor | 3 . . . 236 | 176/234 (74%) | |
| | (VIAF)—Homo | | | |
| | sapiens, 239 aa. | | | |
| | [WO200134798-A1, | | | |
| | May 17, 2001] | | | |
| AAB68507 | Human GTP- | 1 . . . 231 | 137/234 (58%) | 2e−74 |
| | binding associated | 3 . . . 236 | 176/234 (74%) | |
| | protein #7— | | | |
| | Homo sapiens, | | | |
| | 239 aa. | | | |
| | [WO200105970-A2, | | | |
| | Jan. 25, 2001] | | | |

In a BLAST search of public sequence databases, the NOV58a protein was found to have homology to the proteins shown in the BLASTP data in Table 58E.

TABLE 58E
Public BLASTP Results for NOV58a

| | | NOV58a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9CQU4 | 1700010B22RIK | 1 . . . 239 | 208/239 (87%) | e−121 |
| | PROTEIN—Mus | 3 . . . 240 | 229/239 (95%) | |
| | musculus | | | |
| | (Mouse), 240 aa. | | | |
| Q9WUP3 | PDCL2—Mus | 1 . . . 239 | 207/239 (86%) | e−121 |
| | musculus | 1 . . . 238 | 228/239 (94%) | |
| | (Mouse), 238 aa | | | |
| | (fragment). | | | |
| Q9DA99 | 1700016K07RIK | 47 . . . 239 | 165/193 (85%) | 3e−94 |
| | PROTEIN—Mus | 1 . . . 192 | 183/193 (94%) | |
| | musculus | | | |
| | (Mouse), 192 aa. | | | |
| CAC40345 | SEQUENCE 5 | 1 . . . 237 | 133/238 (55%) | 1e−74 |
| | FROM PATENT | 3 . . . 239 | 181/238 (75%) | |
| | W00134798— | | | |
| | Brachydanio | | | |
| | rerio (Zebrafish) | | | |
| | (Zebra danio), | | | |
| | 239 aa. | | | |
| Q9H2J4 | HTPHLP | 1 . . . 231 | 137/234 (58%) | 8e−74 |
| | (UNKNOWN) | 3 . . . 236 | 176/234 (74%) | |
| | (PROTEIN FOR | | | |
| | MGC:3062)— | | | |
| | Homo sapiens | | | |
| | (Human), 239 aa. | | | |

PFam analysis predicts that the NOV58a protein contains the domains shown in the Table 58F.

TABLE 58F
Domain Analysis of NOV58a

| | | Identities/ | |
|---|---|---|---|
| | NOV58a | Similarities for | Expect |
| Pfam Domain | Match Region | the Matched Region | Value |
| Phosducin: domain | 60 . . . 175 | 32/120 (27%) | 5.8 |
| 1 of 1 | | 55/120 (46%) | |

### Example 59

The NOV59 clone was analyzed, and the nucleotide and predicted polypeptide sequences are shown in Table 59A.

TABLE 59A
NOV59 Sequence Analysis

| | SEQ ID NO:181 | 981 bp | |
|---|---|---|---|
| NOV59a, | GCCACCGCGCCCAGCTGGCTTTTGTTTTTTATCCTTCTGCTCCTCATTTACCTATTCA | | |
| CG59576-01 DNA Sequence | | | |
| | CCATCATTGGTACTCTTATGCTQTTCTTTGCCATCAAACTGGATTTCTGCCTGCACAG | | |
| | CTCCTTCTATTTCTTCATCACTGTCCTCTCCTTCCTAGAGATCTGGTATACCACCATC | | |
| | ACCATCCCCAAGATGTTCTTCAACCTACCCAGTGAGCAGAAGACCACCTCCCTGGATG | | |
| | GTTGCCTATTGCAGATGTATTTCTTTTACTCCCTCGGCATCACTGAGGTTTGCTTGCT | | |
| | CACCACCAGGGCTATGGACACATACCTGGCCATCTGTAATCACCTTTGCTACCCCACA | | |
| | GTCACGACACCTCAGCTCTACACTCAGGTGATTCTAGGTTGTTGCATCTGTGGCTTCT | | |
| | TCACGCTGCTCCCTGAGATTGCTTGGATATCCACACFGCCATTTTGTGGTCCAAATCA | | |
| | AATCCACAACATTTTCTGTGACCTTGATCCTATCCTGAATCTAGCATGTGTAGACACT | | |
| | GGCCCAGTTGTTTTAATCAAGGTTGTGGACATTGTACATGCTGTGGAGATCATCACAG | | |
| | CTATAATGCTTGTGACTTTGCCTTACCTCCAAATTATTGCAGTGATCCTAAGAAACTG | | |
| | CTCTGCTGATCGATGCCAAAAGGCATTTTCTACCTATGCTTTCCACCTTGCTATTTTC | | |
| | TTAATCTTTTTTGGAAGTGTAGCCCTGATGTACCTGCTCTTCTCTGCCAAGTACTCCT | | |
| | TTTTCTGGGACACAACCATCAGCCTAATGTTTGCAGTGCTGTCACCGACAACAATCAT | | |
| | CTGTAGTCTCAGGAATAAAGAGATAAAGGAACCAATAAAAAAGCACATGTCCCAATCA | | |
| | ATGATATGCACACATCATGTCAAATAAGACCAAATACACACCTCTTAATTACCAAACA | | |
| | ATATTTATACAAATATTTACATTAATACGTTCAGTGTGTTTGTTGCTGCTGTG | | |
| | ORF Start: GCC at 1 | | ORF Stop: TAA at 895 |
| | SEQ ID NO:182 | 298 aa | MW at 33780.0 kD |

| | |
|---|---|
| NOV59a, | ATAPSWLLFFTLLLLIYLFTIIGSLMVFFAIKLDFCLHSSFYFFTSVLSFLETWYTTI |
| CG59576-01 Protein Sequence | |
| | TIPKMFFNLASEQKTTSLDGCLLQMYFFYSLGITEVCLLTTRAMDRYLAICNHLCYPT |
| | VTTPQLYTQVTLGCCICGFFTLLPETAWISTLPFCGPNQIHNTFCDLDPILNLACVDT |
| | GPVVLIKVVDIVHAVEIITAIMLVTLAYVQIIAVILRNCSADGCQKAFSTYAFHLAIF |
| | LIFFCSVALMYLLFSAKYSFFWDTTTSLMFAVLSPTTITCSLRNKEIKEATKKHMCQS |
| | MICTHHVK |

Further analysis of the NOV59a protein yielded the following properties shown in Table 59B.

TABLE 59B
Protein Sequence Properties NOV59a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Likely cleavage site between residues 25 and 26 |
| analysis: | |

A search of the NOV59a protein against the Geneseq database, a proprietary database that contains sequences published in patents and patent publication, yielded several homologous proteins shown in Table 59C.

TABLE 59C

| | | NOV59a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72586 | Human OR-like | 7 . . . 295 | 286/289 (98%) | e−167 |
| | polypeptide query | 1 . . . 289 | 286/289 (98%) | |
| | sequence, SEQ | | | |
| | ID NO: 2267— | | | |
| | Homo sapiens, | | | |
| | 289 aa. | | | |
| | [WO200127158- | | | |
| | A2, Apr. 19, | | | |
| | 2001] | | | |
| AAG71784 | Human olfactory | 7 . . . 295 | 286/289 (98%) | e−167 |
| | receptor poly- | 1 . . . 289 | 286/289 (98%) | |
| | peptide, SEQ ID | | | |
| | NO: 1465— | | | |
| | Homo sapiens, | | | |
| | 289 aa. | | | |
| | [WO200127158- | | | |
| | A2, Apr. 19, | | | |
| | 2001] | | | |
| AAG71785 | Human olfactory | 5 . . . 292 | 175/293 (59%) | 6e−95 |
| | receptor poly- | 20 . . . 311 | 217/293 (73%) | |
| | peptide, SEQ ID | | | |
| | NO: 1466— | | | |
| | Homo sapiens, | | | |
| | 318 aa. | | | |
| | [WO200127158- | | | |
| | A2, Apr. 19, | | | |
| | 2001] | | | |
| AAU24721 | Human olfactory | 7 . . . 283 | 170/279 (60%) | 4e−94 |
| | receptor | 53 . . . 328 | 212/279 (75%) | |
| | AOLFR220— | | | |
| | Homo sapiens, | | | |
| | 343 aa. | | | |
| | [WO200168805- | | | |
| | A2, Sep. 20, | | | |
| | 2001] | | | |
| AAG71808 | Human olfactory | 7 . . . 283 | 170/279 (60%) | 4e−94 |
| | receptor poly- | 29 . . . 304 | 212/279 (75%) | |
| | peptide, SEQ ID | | | |
| | NO: 1489— | | | |
| | Homo sapiens, | | | |
| | 317 aa. | | | |
| | [WO200127158- | | | |
| | A2, Apr. 19, | | | |
| | 2001] | | | |

In a BLAST search of public sequence databases, the NOV59a protein was found to have homology to the proteins shown in the BLASTP data in Table 59D.

TABLE 59D
Public BLASTP Results for NOV59a

| | | NOV59a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q96R35 | OLFACTORY | 50 . . . 267 | 107/218 (49%) | 7e−55 |
| | RECEPTOR— | 1 . . . 216 | 146/218 (66%) | |
| | Homo sapiens | | | |
| | (Human), 216 aa | | | |
| | (fragment). | | | |
| Q9EPG2 | M51 OLFACTORY | 2 . . . 285 | 115/289 (39%) | 2e−52 |
| | RECEPTOR—Mus | 19 . . . 303 | 172/289 (58%) | |
| | musculus | | | |
| | (Mouse), 314 aa. | | | |
| O95007 | Olfactory receptor | 10 . . . 285 | 109/279 (39%) | 1e−51 |
| | 6B1 (Olfactory | 28 . . . 301 | 170/279 (60%) | |
| | receptor 7-3) | | | |
| | (OR7-3)—Homo | | | |
| | sapiens (Human), | | | |
| | 311 aa. | | | |
| Q9QWU6 | OLFACTORY | 1 . . . 289 | 111/298 (37%) | 2e−50 |
| | RECEPTOR 17— | 20 . . . 314 | 171/298 (57%) | |
| | Mus musculus | | | |
| | (Mouse), 327 aa. | | | |
| P23270 | Olfactory receptor- | 1 . . . 289 | 111/298 (37%) | 2e−50 |
| | like protein 17— | 20 . . . 314 | 171/298 (57%) | |
| | Rattus norvegicus | | | |
| | (Rat), 327 aa. | | | |

PFam analysis predicts that the NOV59a protein contains the domains shown in the Table 59E.

TABLE 59E
Domain Analysis of NOV59a

| | | Identities/ | |
|---|---|---|---|
| | NOV59a | Similarities for | Expect |
| Pfam Domain | Match Region | the Matched Region | Value |
| 7tm_1: domain | 37 . . . 164 | 30/134 (22%) | 5.4e−13 |
| 1 of 1 | | 90/134 (67%) | |

### Example 60

The NOV60 clone was analyzed, and the nucleotide and predicted polypeptide sequences are shown in Table 60A.

TABLE 60A
NOV60 Sequence Analysis

| | SEQ ID NO:183 | 1201 bp | |
|---|---|---|---|
| NOV60a | AGGATAACTTTATATGTTGCAAAATGACTCACATAGTATATTTTATTTAACCAGCCTA | | |
| CG59557-1 DNA Sequence | | | |
| | ATTTCAAGGCTGTTTAGTTGCTTGAAAAGAAGGTTTTTATTTGTTCTTTGCATGTACT | | |
| | TAGAATGCTGACTGTGTTTTATCACCCAACAAGTGAAACCGCTGAAAATATGGATCCA | | |
| | GAGAATCAGACAATGGTGACTGAGTTTTATTTCTCTGATTTTCCTCAATCTAAGAATG | | |
| | GCAGCCTCTTATTCTTCATTCCTATGCTCTTTATTTATATATTCATTCTTGTTGGAAA | | |
| | TTTCATGATTTTCTTTGCTGTCCGACCGCACCCCCATCTCCATAATCCTATGTACACT | | |
| | TTTATCAGTGTCTTCTCCTTCCTGGAGATTTGGTACACCACCGTGACTATCCCCAAGA | | |
| | TGCTCTCCAACCTTCTCAGTGAACAGAAAACCATCTCTTTCATAGCTTGCCTCCTGCA | | |
| | GATGTACTTCTTCCACTCACTCGGGGTCACAGAAGCCCTAGTCCTCACAGTGATGGCC | | |
| | ATTGACAGCTGTGTAGCCATCTGCAACCCCCTTCGCTATGCAATCACTATGTCCCCTA | | |
| | GACTGTGCATCCAGCTCTCCACTGGCTCTTGCATTTTTGGCTTCCTCATGTTACTGCC | | |
| | AGAGATTCTGTGCATTTCCACTCTTCCATTCTGTGGCGCCAACCAAATTCATCAACTC | | |
| | TTTTGTGACTTTGAACCTCTGCTGCAGTTAGCCTGCACAGATACGTACATAATTCTGG | | |
| | TTGAACATGTGATCCGTGCTATTTCCATTCTGACCTCTGTCTCTGTCATCACCCTTTT | | |
| | CTATTTAAGAATCATCACCGTGATCCTGACGATTCCCTCTGGTGAGAGTCGTCACAAG | | |
| | GCTTTCTTCACATGTGCAGCCCACATTGCTATTTTCTTCCTGTTTTTTCGCAGTGTGT | | |
| | CACTCATGTATCTGCGCTTCTCTGTCACATTCCCACCATTACTGGACAAGGCCATTGC | | |
| | ACTGATGTTTGCTGTCCTTGCCCTACTTTTCAACCCAGTAATCTATAGTCTGAGGAAC | | |
| | AAAGATATCAAAAACGCCACCAAGAAAATCCTCTGTTCTCAAAAGATCTTCAATGCCT | | |
| | CTGGGAGCTAATGGAGTTCACACACACCTCTTCAAAGAAATCTCATCATCTCCTTAAG | | |
| | TTTAAAATGCTAACAAATCAGTTTTTTTAAATTACCATGCA | | |
| | ORF Start: ATG at 121 | | ORF Stop: TAA at 1111 |
| | SEQ ID NO:184 | 330 aa | MW at 37439.1 kD |

| | |
|---|---|
| NOV60a, | MLTVFYEPTSETAENMDPENQTN1VTEFYFSDFPQSKNGSLLFPIPMLFIYIFILVGNF |
| CG59557-01 Protein Sequence | |
| | MIFFAVRPDPHLHNPMYSFISVFSFLEIWYTTVVTIPKMLSNLLSEQKTISFIGCLLQM |
| | YFFHSLGVTEALVLTVMAIDRCVAICNPLRYAITMSPRLCIQLSTGSCIFGFLMLLPE |
| | IVCISTLPFCGANQIHQLFCDFEPVLQLACTDTYIILVEDVIRAISILTSVSVITLFY |
| | LRITTVILRIPSGESRQKAFFTCAAHIAIELLFFGSVSLMYLRFSVTFPPLLDKAIAL |
| | MFAVLALLFNPVIYSLRNKDMKNATKKILCSQKMFNASGS |

Further analysis of the NOV60a protein yielded the following properties shown in Table 60B.

TABLE 60B
Protein Sequence Properties NOV60a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.0300 probability |
| | located in mitochondrial inner membrane |
| SignalP | Likely cleavage site between residues 67 and 68 |
| analysis: | |

A search of the NOV60a protein against the Geneseq database, a proprietary database that contains sequences published in patents and patent publication, yielded several homologous proteins shown in Table 60C.

TABLE 60C
Geneseq Results for NOV60a

| | | NOV60a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG71807 | Human olfactory | 16 . . . 330 | 313/315 (99%) | e−180 |
| | receptor poly- | 1 . . . 315 | 314/315 (99%) | |
| | peptide, SEQ ID | | | |
| | NO: 1488— | | | |
| | Homo sapiens, | | | |
| | 319 aa. | | | |
| | [WO200127158- | | | |
| | A2, Apr. 19, | | | |
| | 2001] | | | |
| AAG71803 | Human olfactory | 16 . . . 329 | 219/314 (69%) | e−129 |
| | receptor poly- | 1 . . . 314 | 259/314 (81%) | |
| | peptide, SEQ ID | | | |
| | NO: 1484— | | | |
| | Homo sapiens, | | | |
| | 315 aa. | | | |
| | [WO200127158- | | | |
| | A2, Apr. 19, | | | |
| | 2001] | | | |
| AAU246581 | Human olfactory | 9 . . . 329 | 218/321 (67%) | e−128 |
| | receptor | 10 . . . 330 | 259/321 (79%) | |
| | AOLFR156— | | | |
| | Homo sapiens, | | | |
| | 331 aa. | | | |
| | [WO200168805- | | | |
| | A2, Sep. 20, | | | |
| | 2001] | | | |
| AAU24721 | Human olfactory | 20 . . . 329 | 196/310 (63%) | e−111 |
| | receptor | 33 . . . 342 | 234/310 (75%) | |
| | AOLFR220— | | | |
| | Homo sapiens, | | | |
| | 343 aa. | | | |
| | [WO200168805- | | | |
| | A2, Sep. 20, | | | |
| | 2001] | | | |
| AAG71808 | Human olfactory | 20 . . . 323 | 195/304 (64%) | e−111 |
| | receptor Homo | 9 . . . 312 | 232/304 (76%) | |
| | sapiens, 317 aa. | | | |
| | [WO200127158- | | | |
| | A2, Apr. 19, | | | |
| | 2001] | | | |

In a BLAST search of public sequence databases, the NOV60a protein was found to have homology to the proteins shown in the BLASTP data in Table 60D.

TABLE 60D
Public BLASTP Results for NOV60a

| | | NOV60a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9WU86 | ODORANT | 15 . . . 324 | 135/315 (42%) | 4e-67 |
| | RECEPTOR S1— | 8 . . . 320 | 188/315 (58%) | |
| | Mus musculus | | | |
| | (Mouse), 324 aa. | | | |
| Q9EPG2 | M51 OLFACTORY | 20 . . . 325 | 129/307 (42%) | 4e−65 |
| | RECEPTOR— | 5 . . . 311 | 189/307 (61%) | |
| | Mus musculus | | | |
| | (Mouse), 314 aa. | | | |
| P23270 | Olfactory receptor- | 24 . . . 319 | 126/301 (41%) | 8e−65 |
| | like protein I7— | 10 . . . 310 | 182/301 (59%) | |
| | Rattus norvegicus | | | |
| | (Rat), 327 aa. | | | |
| Q9QWU6 | OLFACTORY | 16 . . . 319 | 128/310 (41%) | 9e−64 |
| | RECEPTOR I7— | 1 . . . 310 | 184/310 (59%) | |
| | Mus musculus | | | |
| | (Mouse), 327 aa. | | | |
| O13036 | CHICK | 16 . . . 319 | 122/305 (40%) | 1e−63 |
| | OLFACTORY | 1 . . . 305 | 187/305 (61%) | |
| | RECEPTOR 7— | | | |
| | Gallus gallus | | | |
| | (Chicken), 323 aa. | | | |

PFam analysis predicts that the NOV60a protein contains the domains shown in the Table 60E.

TABLE 60E
Domain Analysis of NOV60a

| | | Identities/ | |
|---|---|---|---|
| | NOV60a | Similarities for | Expect |
| Pfam Domain | Match Region | the Matched Region | Value |
| 7tm_1: domain | 56 . . . 204 | 45/270 (17%) | 2.4e−21 |
| 1 of 1 | | 172/270 (64%) | |

### Example 61

The NOV61 clone was analyzed, and the nucleotide and predicted polypeptide sequences are shown in Table 61A.

TABLE 61A
NOV61 Sequence Analysis

| | SEQ ID NO:185 | 1061 bp | |
|---|---|---|---|
| NOV61a, | CAATCTGCTCCTAAGTCATCTTTTTCTTTTTCACAGGGAAATGGGGGAAAATCAGACA | | |
| CG59555-01 DNA Sequence | | | |
| | ATGGTCACAGAGTTCCTCCTACTGGGATTTCTCCTGGGCCCAAGGATTCAGATGCTCC | | |
| | TCTTTCGGCTCTTCTCCCTGTTCTATATCTTCACCCTGCTGCGGAACGGGGCCATCCT | | |
| | GGGGCTCATCTCACTGGACTCCAGACTCCACACCCCCATGTACTTCTTCCTCTCACAC | | |
| | CTGGCTGTCGTCGACATCGCCTACACCCGCAACACGGTGCCCCAGATGCTGGCGAACC | | |
| | TCCTGCATCCAGCCAAGCCCATCTCCTTTGCTGGCTGCATGACGCAGACCTTTCTCTG | | |
| | TTTGAGTTTTGGACACAGCCAATGTCTCCTGCTGGTGCTGATGTCCTACGATCGTTAC | | |
| | GTGGCCATCTGCCACCCTCTCCGATACTCCGTCATCATGACCTGGAGAGTCTGCATCA | | |
| | CCCTGGCCGTCACTTCCTCCACCTGTGQCTCCCTCCTGGCTCTGGCCCATGTGGTTCT | | |
| | CATCCTAAGACTCCCCTTCTCTGGGCCTCATCAAATCAACCACTTCTTCTCTGAAATC | | |
| | CTGTCTGTCCTCAGGCTGGCCTGTGCTGACACCTGGCTCAACCAGGTGGTCATCTTTG | | |
| | CAGCCTGCGTGTTCTTCCTGGTGGGGCCACCCAGCCTGGTCCTTGTCTCCTACTCGCA | | |
| | CATCCTGGCGGCCATCCTGAGGATCCAGTCTGGGGAGGGCCGCAGAAAGGCCTTCTCC | | |
| | ACCTGCTCCTCCCACCTCTGCGTGGTGGGACTCTTCTTTGGCAGTGCCATCATCATGT | | |
| | ACATGGCCCCCAAGTCCCGCCATCCTGAGGAGCAGCAAAAGGTCTTTTTTCTATTTTA | | |
| | CAGTTTTTTCAACCCAACACTTAACCCCCTGATTTACAGCCTGAGGAACGGAGAGGTC | | |
| | AAGGGTGCCCTGAGGAGAGCACTGGGCAAGGAAAGTCATTCCTAACTGGTCTGACATT | | |
| | TGACTCTCCCTCCTCACTCATCTCCTCGAATCTTGGTACCAAATACCACCTAAGTTCA | | |
| | CTACTCTCTTTATATCA | | |
| | ORF Start: ATG at 41 | | ORF Stop: TAA at 971 |
| | SEQ ID NO:186 | 310 aa | MW at 34713.8 kD |

| | |
|---|---|
| NOV61a | MGENQTMVTEFLLLGFLLGPRIQMLLFGLFSLFYIFTLLGNGAILGLISLDSRLHTPM |
| CG59555-01 Protein Sequence | |
| | YFFLSHLAVVDIAYTRNTVPQMLANLLHPAKPISFAGCMTQTFLCLSFGHSECLLLVL |
| | MSYDRYVAICHPLRYSVIMTWRVCITLAVTSWTCGSLLALAHVVLILRLPFSGPHEIN |
| | HFFCEILSVLRLACADTWLNQVVIFAACVFFLVGPPSLVLVSYSHILAAILRIQSGEG |
| | RRKAFSTCSSHLCVVGLFFGSAIIMYMAPKSRHPEEQQKVFFLFYSFFNPTLNPLIYS |
| | LRNGEVKGALRRALGKESHS |

Further analysis of the NOV61a protein yielded the following properties shown in Table 61B.

TABLE 61B
Protein Sequence Properties NOV61a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Likely cleavage site between residues 43 and 44 |
| analysis: | |

A search of the NOV61a protein against the Geneseq database, a proprietary database that contains sequences published in patents and patent publication, yielded several homologous proteins shown in Table 61C.

TABLE 61C
Geneseq Results for NOV61a

| | | NOV61a | Identities/ | |
|---|---|---|---|---|
| | Protein/ | Residues/ | Similarities | |
| Geneseq | Organism/Length | Match | for the | Expect |
| Identifier | [Patent #, Date] | Residues | Matched Region | Value |
| AAM29935 | Peptide #3972 | 1 . . . 310 | 310/310 (100%) | 0.0 |
| | encoded by probe | 2 . . . 311 | 310/310 (100%) | |
| | for measuring | | | |
| | placental gene | | | |
| | expression— | | | |
| | Homo sapiens, | | | |
| | 311 aa. | | | |
| | [WO200157272- | | | |
| | A2, Aug. 9, | | | |
| | 2001] | | | |
| AAM17409 | Peptide #3843 | 1 . . . 310 | 310/310 (100%) | 0.0 |
| | encoded by probe | 2 . . . 311 | 310/310 (100%) | |
| | for measuring | | | |
| | cervical gene | | | |
| | expression— | | | |
| | Homo sapiens, | | | |
| | 311 aa. | | | |
| | [WO200157278- | | | |
| | A2, Aug. 9, | | | |
| | 2001] | | | |
| AAG72949 | Human olfactory | 1 . . . 310 | 310/310 (100%) | 0.0 |
| | receptor data | 2 . . . 311 | 310/310 (100%) | |
| | exploration | | | |
| | sequence, SEQ | | | |
| | ID NO: 2631— | | | |
| | Homo sapiens, | | | |
| | 314 aa. | | | |
| | [WO200127158- | | | |
| | A2, Apr. 19, | | | |
| | 2001] | | | |
| AAG72187 | Human olfactory | 1 . . . 310 | 310/310 (100%) | 0.0 |
| | receptor poly- | 1 . . . 310 | 310/310 (100%) | |
| | peptide, SEQ ID | | | |
| | NO: 1868— | | | |
| | Homo sapiens, | | | |
| | 310 aa. | | | |
| | [WO200127158- | | | |
| | A2, Apr. 19, | | | |
| | 2001] | | | |
| AAU04577 | Human G-protein | 1 . . . 310 | 288/310 (92%) | e−165 |
| | coupled receptor | 1 . . . 308 | 294/310 (93%) | |
| | like protein, | | | |
| | GPCR #11— | | | |
| | Homo sapiens, | | | |
| | 308 aa. | | | |
| | [WO200153454- | | | |
| | A2, Jul. 26, 2001] | | | |

In a BLAST search of public sequence databases, the NOV61a protein was found to have homology to the proteins shown in the BLASTP data in Table 61D.

TABLE 61D
Public BLASTP Results for NOV61a

| | | NOV61a | Identities/ | |
|---|---|---|---|---|
| Protein | | Residues/ | Similarities | |
| Accession | Protein/ | Match | for the | Expect |
| Number | Organism/Length | Residues | Matched Portion | Value |
| Q96R46 | OLFACTORY | 67 . . . 283 | 217/217 (100%) | e−125 |
| | RECEPTOR— | 1 . . . 217 | 217/217 (100%) | |
| | Homo sapiens | | | |
| | (Human), 217 aa | | | |
| | (fragment). | | | |
| O95047 | Olfactory | 1 . . . 307 | 217/307 (70%) | e−122 |
| | receptor 2A4— | 1 . . . 307 | 250/307 (80%) | |
| | Homo sapiens | | | |
| | (Human), 310 aa. | | | |
| Q9NQN0 | DJ1005H11.1 | 39 . . . 307 | 187/269 (69%) | e−103 |
| | (7 TRANS- | 1 . . . 269 | 216/269 (79%) | |
| | MEMBRANE | | | |
| | RECEPTOR | | | |
| | (RHODOPSIN | | | |
| | FAMILY) | | | |
| | (OLFACTORY | | | |
| | RECEPTOR | | | |
| | LIKE) | | | |
| | PROTEIN))— | | | |
| | Homo sapiens | | | |
| | (Human), 272 aa | | | |
| | (fragment). | | | |
| Q9Z1V2 | OLFACTORY | 63 . . . 285 | 172/223 (77%) | 9e−98 |
| | RECEPTOR | 1 . . . 223 | 190/223 (85%) | |
| | B12—Mus | | | |
| | musculus | | | |
| | (Mouse), 223 aa | | | |
| | (fragment). | | | |
| O43888 | OLFACTORY | 67 . . . 282 | 173/217 (79%) | 1e−97 |
| | RECEPTOR— | 1 . . . 217 | 188/217 (85%) | |
| | Homo sapiens | | | |
| | (Human), 217 aa | | | |
| | (fragment). | | | |

PFam analysis predicts that the NOV61a protein contains the domains shown in the Table 61E.

TABLE 61E
Domain Analysis of NOV61a

| | | Identities/ | |
|---|---|---|---|
| | NOV61a | Similarities for | Expect |
| Pfam Domain | Match Region | the Matched Region | Value |
| 7tm_1: domain | 40 . . . 289 | 65/269 (24%) | 1.1e−45 |
| 1 of 1 | | 188/269 (70%) | |

### Example 62

The NOV62 clone was analyzed, and the nucleotide and predicted polypeptide sequences are shown in Table 62A.

TABLE 62A
NOV62 Sequence Analysis

| | SEQ ID NO:187 | 1206 bp | |
|---|---|---|---|
| NOV62a, | AGTTGGTTGTAAATAATTCTGCTTATATTACCTACAGAGTAAACATTATAGCATTATC | | |
| CG59551-1 DNA Sequence | | | |
| | ACTCCAGAATCCTTTGTTTCTATGGTTTCCACATCTTTCCAATGTCTAGATGTTCCAG | | |
| | CTGCCCATCTCTGACAAATCCACCTGTGTCTCACAATGGATGCCACAGCCTGTAATGA | | |
| | ATCAGTGGATGGCTCACCCGTCTTCTATCTATTGGGCATCCCCTCTCTGCCAGAGACC | | |
| | TTCTTCCTCCCTGTGTTTTTTATTTTCCTCCTCTTCTACCTTCTCATCCTGATGGCTA | | |
| | ATGCCCTGATCCTGGTGGCCGTGGTGGCAGAGCCCAGCCTCCACAAGCCCATGTACTT | | |
| | CTTTCTGATCAATCTCTCCACCTTCCACATCCTTTTCACCACAACCACTGTCCCCAAG | | |
| | ATGCTGTCCTTATTCTTGCTTGCGGACCGCTTCCTCAGCTTTTCTTCCTGCTTACTGC | | |
| | AGATGTACCTCTTCCAAACTTTTACATGTTCAGAAGCCTTCATCCTGGTGGTCATGGC | | |
| | CTATGACCGCTATGTCGCTATCTGCCACCCACTGCACTACCCTGTCCTCATGAACCCA | | |
| | CAGACCAATGCTACCTTGGCAGCCAGTGCCTGGCTAACTGCCCTCCTCCTGCCCATCC | | |
| | CAGCACTACTAAGGACCTCCCAGATGGCATATAACAGCATTGCCTACATCTACCACTG | | |
| | CTTCTGTCATCATCTGGCTGTGGTCCAGGCCTCCTGCTCTGACACCACCCCCCAGACC | | |
| | CTCATGGGCTTCTGCATCGCCATGGTGGTGTCCTTCCTCCCCCTTCTCCTGGTGCTTC | | |
| | TCTCCTATGTCCACATCCTGGCCTCAGTGCTTCGCATCAGTTCCCTAGAAGGACGGCC | | |
| | AAAAGCCTTCTCCACCTGCAGCTCCCACCTTCTGGTCGTGGGCACCTACTACTCATCT | | |
| | ATTGCCATAGCCTACGTGGCCTACACGGCTGACCTGCCCCTTGACTTCCATATCATGG | | |
| | GCAATGTGGTATATGCCATTCTCACACCAATTCTCAACCCCCTCATTTACACGCTGAG | | |
| | AAACAGCGATGTAAAGGCAGCCATCACCAAAATCATGTCTCAAGACCCACGCTGTGAC | | |
| | AGGAGCATTTGACCTTTAAATGCAGCTAACTCTGCTTCCAGGACACCAAATAACAGTG | | |
| | CTTAGCACAGAGAAAGGACTCAATACATGATAATGAAATAA | | |
| | ORF Start: ATG at 152 | | ORF Stop: TGA at 1112 |
| | SEQ ID NO:188 | 320 aa | MW at 35502.6 kD |

| | |
|---|---|
| NOV62a, | MDATACNESVDGSPVFYLLGIPSLPETFFLPVFFIFLLFYLLILMGNALILVAVVAEP |
| CG59551-1 Protein Sequence | |
| | SLHKPMYFFLINLSTLDILFTTTTVPKMLSLFLLGDRFLSFSSCLLQMYLFQSFTCSE |
| | AFILVVMAYDRYVAICHPLHYPVLMNPQTNATLAASAWLTALLLPIPAVVRTSQMAYN |
| | SIAYIYHCFCDHLAVVQASCSDTTPQTLMGFCIAMVVSFLPLLLVLLSYVHILASVLR |
| | ISSLEGRAKAFSTCSSELLVVGTYYSSIAIAYVAYRADLPLDFHIMGNVVYAILTPIL |
| | NPLIYTLRNRDVKAAITKIMSQDPGCDRSI |

Further analysis of the NOV62a protein yielded the following properties shown in Table 62B.

TABLE 62B
Protein Sequence Properties NOV62a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.3000 probability |
| | located in microbody (peroxisome) |
| SignalP | Likely cleavage site between residues 57 and 58 |
| analysis: | |

A search of the NOV62a protein against the Geneseq database, a proprietary database that contains sequences published in patents and patent publication, yielded several homologous proteins shown in Table 62C.

TABLE 62C
Geneseq Results for NOV62a

| | | NOV62a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72119 | Human olfactory | 35 . . . 290 | 213/256 (83%) | e−119 |
| | receptor poly- | 2 . . . 257 | 228/256 (88%) | |
| | peptide, SEQ ID | | | |
| | NO: 1800— | | | |
| | Homo sapiens, | | | |
| | 295 aa. | | | |
| | [WO200127158- | | | |
| | A2, Apr. 19, | | | |
| | 2001] | | | |
| AAU24639 | Human olfactory | 16 . . . 308 | 129/293 (44%) | 6e−67 |
| | receptor | 17 . . . 308 | 186/293 (63%) | |
| | AOLFR134— | | | |
| | Homo sapiens, | | | |
| | 325 aa. | | | |
| | [WO200168805- | | | |
| | A2, Sep. 20, | | | |
| | 2001] | | | |
| AAG72479 | Human OR-like | 16 . . . 308 | 129/293 (44%) | 6e−67 |
| | polypeptide query | 17 . . . 308 | 186/293 (63%) | |
| | sequence, SEQ | | | |
| | ID NO: 2160— | | | |
| | Homo sapiens, | | | |
| | 324 aa. | | | |
| | [WO200127158- | | | |
| | A2, Apr. 19, | | | |
| | 2001] | | | |
| AAG71590 | Human olfactory | 16 . . . 308 | 129/293 (44%) | 6e−67 |
| | receptor poly- | 17 . . . 308 | 186/293 (63%) | |
| | peptide, SEQ ID | | | |
| | NO: 1271— | | | |
| | Homo sapiens, | | | |
| | 324 aa. | | | |
| | [WO200127158- | | | |
| | A2, Apr. 19, | | | |
| | 2001] | | | |
| AAG71632 | Human olfactory | 16 . . . 315 | 126/300 (44%) | 3e−64 |
| | receptor Homo | 13 . . . 312 | 179/300 (59%) | |
| | sapiens, 316 aa. | | | |
| | [WO200127158- | | | |
| | A2, Apr. 19, | | | |
| | 2001] | | | |

In a BLAST search of public sequence databases, the NOV62a protein was found to have homology to the proteins shown in the BLASTP data in Table 62D.

TABLE 62D
Public BLASTP Results for NOV62a

| | | NOV62a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9Z236 | OLFACTORY | 70 . . . 289 | 187/220 (85%) | e−104 |
| | RECEPTOR— | 2 . . . 221 | 202/220 (91%) | |
| | Rattus norvegicus | | | |
| | (Rat), 221 aa | | | |
| | (fragment). | | | |
| CAB43131 | OLFACTORY | 69 . . . 240 | 136/172 (79%) | 1e−73 |
| | RECEPTOR— | 1 . . . 172 | 148/172 (85%) | |
| | Stenella | | | |
| | coeruleoalba | | | |
| | (Striped dolphin), | | | |
| | 172 aa | | | |
| | (fragment). | | | |
| Q9EPG2 | M51 | 16 . . . 310 | 131/295 (44%) | 2e−67 |
| | OLFACTORY | 12 . . . 305 | 191/295 (64%) | |
| | RECEPTOR— | | | |
| | Mus musculus | | | |
| | (Mouse), 314 aa. | | | |
| Q9H208 | HP4 | 16 . . . 312 | 127/297 (42%) | 3e−65 |
| | OLFACTORY | 12 . . . 308 | 180/297 (59%) | |
| | RECEPTOR— | | | |
| | Homo sapiens | | | |
| | (Human), 317 aa | | | |
| | (fragment). | | | |
| Q920G5 | OLFACTORY | 16 . . . 308 | 126/295 (42%) | 1e−62 |
| | RECEPTOR | 19 . . . 311 | 180/295 (60%) | |
| | P3—Mus | | | |
| | musculus | | | |
| | (Mouse), 324 aa. | | | |

PFam analysis predicts that the NOV62a protein contains the domains shown in the Table 62E.

TABLE 62E
Domain Analysis of NOV62a

| | | Identities/ | |
|---|---|---|---|
| | NOV62a | Similarities for | Expect |
| Pfam Domain | Match Region | the Matched Region | Value |
| 7tm_1: domain | 46 . . . 295 | 58/268 (22%) | 4.6e−38 |
| 1 of 1 | | 179/268 (67%) | |

### Example 63

The NOV63 clone was analyzed, and the nucleotide and predicted polypeptide sequences are shown in Table 63A.

TABLE 63A
NOV63 Sequence Analysis

| | SEQ ID NO:189 | 1042 bp | |
|---|---|---|---|
| NOV63a, | GACCTTTCATCACACTCTGGTCATTTACAAACTGTTATTAAGGAATGGGGGACAAGCA | | |
| CG59540-01 DNA Sequence | | | |
| | GCCCTGGGTCACAGAATTCATCCTGGTTGGATTCCAGCTCAGTGCAGAGATGGAGATC | | |
| | TTTCTCTCTTGCATCTTCTCCCTGTTATATCTCTTCAGTCTACTGAGGAATGGCATGA | | |
| | ACATGGGACTCATCTGTCTGGATCCCAGACTACACACCCCCATATACTTCTTCCTGTC | | |
| | ACACTTGGCCGTCATTGACATATACTATGCTTCCAACAATTTGCTCAACATGCTCGAA | | |
| | AACCTAGTGAAACACAAAAAAACTATCTCGTTCATCTCTTGCATTATGCAGATGGCTT | | |
| | TGTATTTGACTTTTGCTGCTGCAGTGTGCATGATTTTGGTGGTGATGTCCTATGACAG | | |
| | ATTTGTCGCGATCTGCCATCCCCTGCATTACACTGTCATCATGAACTGGAGAGTGTGC | | |
| | ACAGTACTGGCTATTACTTCCTGGGCATCTGCATTTTCCCTGGCCCTCATAAATCTAA | | |
| | TTCTCCTTCTAAGGCTGCCCTTCTGTGGGCCCCAGGAGGTGAACCACTTCTTCGGTGA | | |
| | AATTCTGTCTGTCCTCAAACTGGCCTGTGCAGACACCTGGATTAATCAAATTTTTGTC | | |
| | TTTGCTGGTGGTGTGTTTGTCTTAGTCGGGCCCCTTTCCTTGATGCTGATCTCCTACA | | |
| | TGCGCATCCTCTTGGCCATCCTGAAGATCCAGTCAGGCGAGGGCCACAGAAAGCACTT | | |
| | CTCTACCTGCTCCTCCCACCTCTGTGTGCTGGCGTTCTTCTTTGCCAACGCCATTGTC | | |
| | ATGTACATGGCCCCCAAGTCCCGCCATCCCGAGGAGCAGCAGAAGGTCCTTTCCCTGT | | |
| | TTTGCAGCCTTTCGAATCAGGTGCTGAACCCCCCTCTGATCTACAGCTTGACGAATGC | | |
| | AGAGGTCAAGAGTGCCCCACAAGAGGGCCACTGAAGAAGGAGAGGCTGATGTTACAAT | | |
| | CTCAAAGGCACCACGAGGAGAGGGCCTGCTCCGACAAATGGGGAAGTTGGCTTTTT | | |
| | ORF Start: ATG at 45 | | ORF Stop: TGA at 960 |
| | SEQ ID NO:190 | 305 aa | MW at 345548 kD |

| | |
|---|---|
| NOV63a, | MGDKQPWVTEFILVGFQLSAEMEIFLSCIFSLLYLFSLLRNGMNMGLICLDPRLHTPI |
| CG59540-1 Protein Sequence | |
| | YFFLSHLAVIDIYYASNNLLNMLENLVKHKKTISFISCIMQMALYLTFAAAVCMILVV |
| | MSYDRFVAICHPLHYTVIMNWRVCTVLAITSWACGFSLALINLILLLRLPFCGPQEVN |
| | HFFGEILSVLKLACADTWINEIFVFAGGVFVLVGPLSLMLISYMRTLLAILKIQSGEG |
| | HRKDFSTCSSHLCVVGFFFANAIVMYMAPKSRHPEEQQKVLSLFCSLWNQVLNPPLIY |
| | SLRNAEVKSAPQEGH |

Further analysis of the NOV63a protein yielded the following properties shown in Table 63B.

TABLE 63B
Protein Sequence Properties NOV63a

| | |
|---|---|
| PSort | 0.6000 probability located in plasma membrane; 0.4000 |
| analysis: | probability located in Golgi body; 0.3000 probability |
| | located in endoplasmic reticulum (membrane); 0.3000 |
| | probability located in microbody (peroxisome) |
| SignalP | Likely cleavage site between residues 43 and 44 |
| analysis: | |

A search of the NOV63a protein against the Geneseq database, a proprietary database that contains sequences published in patents and patent publication, yielded several homologous proteins shown in Table 63C.

TABLE 63C
Geneseq Results for NOV63a

| | | NOV63a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAU24758 | Human olfactory | 1 . . . 300 | 258/300 (86%) | e−146 |
| | receptor | 1 . . . 299 | 275/300 (91%) | |
| | [WO200168805-A2, | | | |
| | Sep. 20, 2001] | | | |
| AAG72952 | Human olfactory | 1 . . . 300 | 255/300 (85%) | e−144 |
| | receptor data | 1 . . . 299 | 272/300 (90%) | |
| | exploratorium | | | |
| | sequence, SEQ ID | | | |
| | NO: 2634— | | | |
| | Homo sapiens, | | | |
| | 310 aa. | | | |
| | [WO200127158-A2, | | | |
| | Apr. 19, 2001] | | | |
| AAG72377 | Human OR-like | 1 . . . 300 | 255/300 (85%) | e−144 |
| | polypeptide query | 1 . . . 299 | 272/300 (90%) | |
| | sequence, SEQ ID | | | |
| | NO: 2058— | | | |
| | Homo sapiens, | | | |
| | 312 aa. | | | |
| | [WO200127158-A2, | | | |
| | Apr. 19, 2001] | | | |
| AAG72169 | Human olfactory | 1 . . . 300 | 255/300 (85%) | e−144 |
| | receptor poly- | 1 . . . 299 | 272/300 (90%) | |
| | peptide, SEQ ID | | | |
| | NO: 1850— | | | |
| | Homo sapiens, | | | |
| | 312 aa. | | | |
| | [WO200127158-A2, | | | |
| | Apr. 19, 2001] | | | |
| AAG71994 | Human olfactory | 1 . . . 300 | 225/300 (75%) | e−129 |
| | receptor poly- | 1 . . . 299 | 256/300 (85%) | |
| | peptide, SEQ ID | | | |
| | NO: 1675— | | | |
| | Homo sapiens, | | | |
| | 314 aa. | | | |
| | [WO200127158-A2, | | | |
| | Apr. 19, 2001] | | | |

In a BLAST search of public sequence databases, the NOV63a protein was found to have homology to the proteins shown in the BLASTP data in Table 63D.

TABLE 63D
Public BLASTP Results for NOV63a

| | | NOV63a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| O95047 | Olfactory receptor | 1 . . . 299 | 173/299 (57%) | 2e−92 |
| | 2A4—Homo | 1 . . . 298 | 217/299 (71%) | |
| | sapiens (Human), | | | |
| | 310 aa. | | | |
| O43885 | OLFACTORY | 67 . . . 281 | 154/216 (71%) | 5e−88 |
| | RECEPTOR— | 1 . . . 216 | 182/216 (83%) | |
| | Homo sapiens | | | |
| | (Human), 217 aa | | | |
| | (fragment). | | | |
| O43888 | OLFACTORY | 67 . . . 281 | 153/216 (70%) | 8e−88 |
| | RECEPTOR— | 1 . . . 216 | 182/216 (83%) | |
| | Homo sapiens | | | |
| | (Human), 217 aa | | | |
| | (fragment). | | | |
| Q96R48 | OLFACTORY | 67 . . . 281 | 153/216 (70%) | 2e−87 |
| | RECEPTOR— | 1 . . . 216 | 181/216 (82%) | |
| | Homo sapiens | | | |
| | (Human), 217 aa | | | |
| | (fragment). | | | |
| Q96R47 | OLFACTORY | 67 . . . 281 | 149/215 (69%) | 3e−84 |
| | RECEPTOR— | 1 . . . 214 | 175/215 (81%) | |
| | Homo sapiens | | | |
| | (Human), 215 aa | | | |
| | (fragment). | | | |

PFam analysis predicts that the NOV63a protein contains the domains shown in the Table 63E.

TABLE 63E
Domain Analysis of NOV63a

| | | Identities/ | |
|---|---|---|---|
| | NOV63a | Similarities for | Expect |
| Pfam Domain | Match Region | the Matched Region | Value |
| 7tm_1: domain | 47 . . . 290 | 55/270 (20%) | 9.7e−25 |
| 1 of 1 | | 174/270 (64%) | |

### Example 64

The NOV64 clone was analyzed, and the nucleotide and predicted polypeptide sequences are shown in Table 64A.

TABLE 64A
NOV64 Sequence Analysis

| | SEQ ID NO:191 | 973 bp | |
|---|---|---|---|
| NOV64a, | AGGCACTAAATGAATATCTGTTTAATTCATAAAGTAACAGAGTTTCTCTTCTCTGGAT | | |
| CG59280-01 DNA Sequence | | | |
| | TCCCACAGTTTGAAGATGGTAGCCTCCTCTTCTTCATTCCATTGTTTGTTATCTACAT | | |
| | ATTCATTCTCATTGGGAATCTTATTCTATTTTTTCCACTCAGGGTGGATACCCGTCTC | | |
| | CACAACCCCATGTATAATTTTATCAGCATTTTCTCATTTCTGGAGATCTGGTACACAA | | |
| | CTGCCACAATTCCCAAGATGCTCTCCATCCTCATCAGCAGGCAGAGGACCATCTCCAT | | |
| | GGTTGGTTGCCTCTTGCAGATGTACTTCTTCCATTCACTGGGAAATTCAGAGGGGATT | | |
| | CAACCATCATGACCCCCGGGCTCTGTGTTCAGCTCTCTGTGGGGTCCTGCATCTTTGG | | |
| | CTTTCTTGTGTTGCTCCCAGAGATTGCATGGATTTCCACACTGCCCTTCTGTGGACCC | | |
| | AACCAAATCCACCAGATCTTCTGTGATTTTGAACCTGTGCTGCGCTTGGCCTGTACAG | | |
| | ACACGTCCATGATTCTGATTGAGGATGTGATCCATGCTGTGGCCATTGTATTCTCTGT | | |
| | CCTGATTATTGCCTTTTCTTATATCAGAATCATCACTGTAATCCTGAGGATTCCCTCT | | |
| | GTTGAAGGCCGCCAGAAGGCCTTTTCTACCTGTGCCCCCCATCTTACTGTCTTTCTGA | | |
| | TGTTCTATGGCAGTGTATCCCTCATGTACCTGCGTTTCTCTGCCACTTTCCCACCGAT | | |
| | TTTGCACACAGCTGTTGCACTGATGTTTGCAGTTCTTGCTCCCTTTTTCAACCCTATC | | |
| | ATCTATAGCTTTAGAAATAAGGACATGAAGATTCCAATTAAAAAGCTTTTCTGCCCTC | | |
| | AGAAGATGGTTAATTTATCTGTAGATTAATGCTAGCTCATAGGCA | | |
| | ORF Start: ATG at 10 | | ORF Stop: TAA at 955 |
| | SEQ ID NO:192 | 315 aa | MW at 35741.4 kD |
| NOV64a, | MNICLIHKVTEFLFSGFPQFEDGSLLFFIPLFVIYIFIVIGNLIVFFAVRVDTRLHNP | | |
| CG59280-01 Protein Sequence | | | |
| | MYNFISIFSFLEIWYTTATIPKMLSILISRQRTISMVGCLLQMYFFHSLGNSEGILLT | | |
| | TMAIDRYVAICNPLRYPTIMTPGLCVQLSVGSCIFGFLVLLPEIAWISTLPFCGPNQI | | |
| | HQIFCDFEPVLRLACTDTSMILTEDVTHAVAIVFSVLIIAFSYIRTTTVTLRTPSVEG | | |
| | RQKAFSTCAAHLSVFLMFYGSVSLMYLRFSATFPPILDTAVALMFAVLAPFFNPIIYS | | |
| | FRNKDMKTAIKKLFCPQKMVNLSVD | | |
| | SEQ ID NO:193 | 929 bp | |
| NOV64b, | TCTTCTTCATTCCATTGTTTGTTATCTACATATTCATTGTCATTGGGAATCTTATTGT | | |
| CG59280-02 DNA Sequence | | | |
| | ATTTTTTGCAGTCAGGGTGGATACCCGTCTCCACAACCCCATGTATAATTTTATCAGC | | |
| | ATTTTCTCATTTCTGGAGATCTGGTACACAACTGCCACAATTCCCAAGATGCTCTCCA | | |
| | TCCTCATCAGCAGGCAGAGGACCATCTCCATGGTTGGTTGCCTCTTGCAGATGTACTT | | |
| | CTTCCATTCACTGGGAAATTCAGAGGGCATTTTGTTGACCACCATGGCCATTGATAGG | | |
| | TACGTTGCCATCTGTAACCCTCTCCGCTACCCAACCATCATGACCCCCGGGCTCTGTG | | |
| | TTCAGCTCTCTGTGGGGTCCTGCATCTTTGGCTTTCTTGTGTTGCTCCCAGAGATTGC | | |
| | ATGGATTTCCACACTGCCCTTCTGTGGACCCAACCAAATCCACCAGATCTTCTGTGAT | | |
| | TTTCAACCTGTGCTGCGCTTGGCCTGTACAGACACGTCCATGATTCTGATTGAGGATC | | |
| | TGATCCATGCTGTGGCCATTGTATTCTCTGTCCTGATTATTGCCTTTTCTTATATCAG | | |
| | AATCATCACTGTAATCCTGAGGATTCCCTCTGTTGAAGGCCGCCAGAAGGCCTTTTCT | | |
| | ACCTGTCCCGCCCATCTTACTGTCTTTCTGATGTTCTATGGCAGTGTATCCCTCATGT | | |
| | ACCTGCGTTTCTCTCCCACTTTCCCACCGATTTTGGACACAGCTGTTGCACTGATGTT | | |
| | TGCAGTTCTTGCTCCCTTTTTCAACCCTATCATCTATAGCTTTACAAATAAGGACATG | | |
| | AAGATTCCAATTAAAAAGCTTTTCTGCCCTCAGAAGATCGTTAATTTATCTGTAGATT | | |
| | AATGCTAGCTCATAGGCACCTTTCACTGTGGATGTTACTCTAACACAATAAACCATAT | | |
| | A | | |
| | ORF Start: TTC at 3 | | ORF Stop: TAA at 870 |
| | SEQ ID NO:194 | 289 aa | MW at 32772.9 kD |

| | |
|---|---|
| NOV64b, | FFIPLFVIYIFIVIGNLIVFFAVRVDTRLHNPMYNFISIFSFLEIWYTTATIPKMLSI |
| CG59280-02 Protein Sequence | |
| | LISRQRTISMVGCLLQMYFFHSLGNSEGILLTTMAIDRYVAICNPLRYPTIMTPGLCV |
| | QLSVGSCIFGFLVLLPEIAWISTLPFCGPNQIHQIFCDFEPVLRLACTDTSMILIEDV |
| | IHAVAIVFSVLIIAFSYTRIITVILRIPSVEGRQKAFSTCAAHLSVFLMFYCSVSLMY |
| | LRFSATFPPILDTAVALMFAVLAPFFNPIIYSFRNKDMKIAIKKLFCPQKMVNLSVD |

Sequence comparison of the above protein sequences yields the following sequence relationships shown in Table 64B.

TABLE 64B
Comparison of NOV64a against NOV64b.

| Protein | NOV64a Residues/ | Identities/Similarities |
|---|---|---|
| Sequence | Match Residues | for the Matched Region |
| NOV64b | 27 . . . 315 | 289/289 (100%) |
| | 1 . . . 289 | 289/289 (100%) |

Further analysis of the NOV64a protein yielded the following properties shown in Table 64C.

TABLE 64C
Protein Sequence Properties NOV64a

| | |
|---|---|
| PSort | 0.6000 probability located in plasma membrane; 0.4000 |
| analysis: | probability located in Golgi body; 0.3000 probability |
| | located in endoplasmic reticulum (membrane); 0.3000 |
| | probability located in microbody (peroxisome) |
| SignalP | Likely cleavage site between residues 54 and 55 |
| analysis: | |

A search of the NOV64a protein against the Geneseq database, a proprietary database that contains sequences published in patents and patent publication, yielded several homologous proteins shown in Table 64D.

TABLE 64D
Geneseq Results for NOV64a

| | | NOV64a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG71805 | Human olfactory | 59 . . . 314 | 255/256 (99%) | e−145 |
| | receptor poly- | 1 . . . 256 | 255/256 (99%) | |
| | peptide, SEQ ID | | | |
| | NO: 1486— | | | |
| | Homo sapiens, | | | |
| | 256 aa. | | | |
| | [WO200127158- | | | |
| | A2, Apr. 19, | | | |
| | 2001] | | | |
| AAG71803 | Human olfactory | 9 . . . 311 | 243/303 (80%) | e−143 |
| | receptor SEQ ID | 9 . . . 311 | 267/303 (87%) | |
| | NO: 1484— | | | |
| | Homo sapiens, | | | |
| | 315 aa. | | | |
| | [WO200127158- | | | |
| | A2, Apr. 19, | | | |
| | 2001] | | | |
| AAU24658 | Human olfactory | 9 . . . 311 | 240/303 (79%) | e−140 |
| | receptor | 25 . . . 327 | 264/303 (86%) | |
| | AOLFR156— | | | |
| | Homo sapiens, | | | |
| | 331 aa. | | | |
| | [WO200168805- | | | |
| | A2, Sep. 20, | | | |
| | 2001] | | | |
| AAG71807 | Human olfactory | 9 . . . 313 | 222/305 (72%) | e−131 |
| | receptor poly- | 9 . . . 313 | 259/305 (84%) | |
| | peptide, SEQ ID | | | |
| | NO: 1488— | | | |
| | Homo sapiens, | | | |
| | 319 aa. | | | |
| | [WO200127158- | | | |
| | A2, Apr. 19, | | | |
| | 2001] | | | |
| AAU24721 | Human olfactory | 9 . . . 308 | 209/300 (69%) | e−119 |
| | receptor | 37 . . . 336 | 242/300 (80%) | |
| | AOLFR220— | | | |
| | Homo sapiens, | | | |
| | 343 aa. | | | |
| | [WO200168805- | | | |
| | A2, Sep. 20, | | | |
| | 2001] | | | |

In a BLAST search of public sequence databases, the NOV64a protein was found to have homology to the proteins shown in the BLASTP data in Table 64E.

TABLE 64E
Public BLASTP Results for NOV64a

| | | NOV64a | Identities/ | |
|---|---|---|---|---|
| Protein | | Residues/ | Similarities for | |
| Accession | Protein | Match | the Matched | Expect |
| Number | Organism/Length | Residues | Portion | Value |
| Q9EPG2 | M51 OLFACTORY | 1 . . . 302 | 137/303 (45%) | 2e−71 |
| | RECEPTOR—Mus | 4 . . . 303 | 194/303 (63%) | |
| | musculus (Mouse), | | | |
| | 314 aa. | | | |
| Q9EPV0 | M50 OLFACTORY | 6 . . . 302 | 132/298 (44%) | 3e−71 |
| | RECEPTOR | 4 . . . 301 | 191/298 (63%) | |
| | (OLFACTORY | | | |
| | RECEPTOR | | | |
| | M50)—Mus | | | |
| | musculus (Mouse), | | | |
| | 316 aa. | | | |
| Q9EPG1 | M50 OLFACTORY | 6 . . . 302 | 130/298 (43%) | 2e−70 |
| | RECEPTOR— | 4 . . . 301 | 190/298 (63%) | |
| | Mus musculus | | | |
| | (Mouse), 316 aa. | | | |
| Q9WU86 | ODORANT | 1 . . . 310 | 133/313 (42%) | 4e−69 |
| | RECEPTOR S1— | 12 . . . 321 | 190/313 (60%) | |
| | Mus musculus | | | |
| | (Mouse), 324 aa. | | | |
| Q96KK4 | DJ994E9.5 | 9 . . . 314 | 137/307 (44%) | 9e−68 |
| | (OLFACTORY | 2 . . . 306 | 189/307 (60%) | |
| | RECEPTOR, | | | |
| | FAMILY 10, | | | |
| | SUBFAMILY C, | | | |
| | MEMBER 1 | | | |
| | (HS6M1-17))— | | | |
| | Homo sapiens | | | |
| | (Human), 306 aa. | | | |

PFam analysis predicts that the NOV64a protein contains the domains shown in the Table 64F.

TABLE 64F
Domain Analysis of NOV64a

| | | Identities/ | |
|---|---|---|---|
| | NOV64a | Similarities for | Expect |
| Pfam Domain | Match Region | the Matched Region | Value |
| 7tm_1: domain | 41 . . . 289 | 51/269 (19%) | 2.2e−33 |
| 1 of 1 | | 179/269 (67%) | |

### Example 65

The NOV65 clone was analyzed, and the nucleotide and predicted polypeptide sequences are shown in Table 65A.

TABLE 65A
NOV65 Sequence Analysis

| | SEQ ID NO:195 | 972 bp |
|---|---|---|
| NOV65a, | GCATGGTGATCCTGTCCTGGGAAAACCAAACGATGAGAGTGGAATTCGTGCTTCAAGG | |
| CG59568-01 DNA Sequence | ATTCTCTTCCATCAGACAGTTAAATATTTTCCTCTTTATGATAATTTTAGTTTTCTAC | |
| | ATCTTAACTGTTTCTGCAAACATCCTCATTGTCCTTCTAGTTTTAGTCAGACATCATC | |
| | TCCACACCCCTATGTACTTCCTCCTGGTGAACTTGTCCTGTCTGGAGATCTGGTATAC | |
| | CTCTAACATCATCCCCAAAATGTTCCTGATTATCATACCTGAAGAGAAGACTATCTCT | |
| | GTGGCTGGCTGGCTGGCACAATTCTACTTCTTCGGATCCCTGGCTGCCACGGAGTGCC | |
| | TCTTCCTCACTGTGATGTCCTATGATCGCTACCTAGCCATCTGCCAGCCTCTTTGCTA | |
| | CCGTGTCCTCATGACTGGCCCCCTTTGCATCAGGCTAGCTGCTGGCTCTTGCTTCTGC | |
| | TGCTTCCTCCTTACAGCAATCACCATGGTCTTGCTATGTAGACTAACCTTCTGTCGAC | |
| | CCTATGAAACTGATCACTTCTTTTGTGACTTCACCCCTCTGCTTCATCTCTCCTGCAT | |
| | GGATACCTCAGTGACTGAGACCATTGCCTTTGCCACCTCTTCTGCAGTAACTCTGATC | |
| | CCATTTCTCCTCATTGTAGCCTCCTACTCCTCCGTCCTTTCTGCTATCCTAAGAATCC | |
| | CATCTTGCACAGGCCAGAAAAAGGCCTTCTCCACCTGCTCTTCCCACCTCACTGTGGT | |
| | CATACTGTTTTATGGGACACTGATTGCCACATACCTTGTGCCCTCAGCCAACTCATCC | |
| | CAACTCTTGTGCAAAGGGTCCTCTCTGCTCTACATCATCCTGACACCCATGTTTAACC | |
| | CCATCATTTATAGCCTGAGAAATAGAGACATCCATGAAGCTCTGAAGAACTGCTTGAG | |
| | GAAGAAGTCAGGTGTTTGCCTTAGATAATACGAAAAGGAAAAAA | |
| | ORF Start: ATG at 3 | ORF Stop: TAA at 954 |
| | SEQ ID NO:196 | 317 aa MW at 35713.4 kD |

| | |
|---|---|
| NOV65a, | MVILSWENQTMRVEFVLQGFSSIRQLNIFLFMIILVFYILTVSGNILIVLLVLVRHHL |
| CG59568-01 Protein Sequence | HTPMYFLLVNLSCLETWYTSNYTPKMLLIIIAEEKTISVAGWLAQFYFFGSLAATECL |
| | LLTVMSYDRYLATCQPLCYRVLNITGPLCIRLAAGSWFCCFLLTAITMVLLCRLTFCGP |
| | YETDHFFCDFTPLVHLSCMDTSVTETTAFATSSAVTLTPFLLIVASYSCVLSATLRIP |
| | SCTGQKKAFSTCSSHLTVVIVFYGTLIATYLVPSANSSQLLCKGSSLLYIILTPMFNP |
| | IIYSLRDIHEALKKCLRKKSGVCLR |

Further analysis of the NOV65a protein yielded the following properties shown in Table 65B.

TABLE 65B
Protein Sequence Properties NOV65a

| | |
|---|---|
| PSort | 0.6000 probability located in plasma membrane; 0.4000 |
| analysis: | probability located in Golgi body; 0.3888 probability located |
| | in mitochondrial inner membrane; 0.3030 probability located |
| | in mitochondrial intermembrane space |
| SignalP | Likely cleavage site between residues 45 and 46 |
| analysis: | |

A search of the NOV65a protein against the Geneseq database, a proprietary database that contains sequences published in patents and patent publication, yielded several homologous proteins shown in Table 65C.

TABLE 65C
Geneseq Results for NOV65a

| | | NOV65a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72527 | Human OR-like | 1 . . . 316 | 315/316 (99%) | 0.0 |
| | polypeptide query | 1 . . . 316 | 315/316 (99%) | |
| | sequence, SEQ ID | | | |
| | NO: 2208— | | | |
| | Homo sapiens, | | | |
| | 316 aa. | | | |
| | [WO200127158- | | | |
| | A2, Apr. 19, | | | |
| | 2001] | | | |
| AAG72231 | Human olfactory | 1 . . . 316 | 315/316 (99%) | 0.0 |
| | receptor poly- | 1 . . . 316 | 315/316 (99%) | |
| | peptide, SEQ ID | | | |
| | NO: 1912— | | | |
| | Homo sapiens, | | | |
| | 316 aa. | | | |
| | [WO200127158- | | | |
| | A2, Apr. 19, | | | |
| | 2001] | | | |
| AAG72084 | Human olfactory | 1 . . . 316 | 315/316 (99%) | 0.0 |
| | receptor poly- | 1 . . . 316 | 315/316 (99%) | |
| | peptide, SEQ ID | | | |
| | NO: 1765— | | | |
| | Homo sapiens, | | | |
| | 316 aa. | | | |
| | [WO200127158- | | | |
| | A2, Apr. 19, | | | |
| | 2001] | | | |
| AAG72700 | Murine OR-like | 1 . . . 308 | 154/308 (50%) | 2e−83 |
| | polypeptide query | 3 . . . 308 | 208/308 (67%) | |
| | sequence, SEQ ID | | | |
| | NO: 2382—Mus | | | |
| | musculus, 314 aa. | | | |
| | [WO200127158- | | | |
| | A2, Apr. 19, | | | |
| | 2001] | | | |
| AAG71814 | Human olfactory | 8 . . . 311 | 142/304 (46%) | 7e−79 |
| | receptor poly- | 5 . . . 308 | 208/304 (67%) | |
| | peptide, SEQ ID | | | |
| | NO: 1495— | | | |
| | Homo sapiens, | | | |
| | 317 aa. | | | |
| | [WO200127158- | | | |
| | A2, Apr. 19, | | | |
| | 2001] | | | |

In a BLAST search of public sequence databases, the NOV65a protein was found to have homology to the proteins shown in the BLASTP data in Table 65D.

TABLE 65D
Public BLASTP Results for NOV65a

| | | NOV65a | Identities/ | |
|---|---|---|---|---|
| Protein | | Residues/ | Similarities for | |
| Accession | Protein | Match | the Matched | Expect |
| Number | Organism/Length | Residues | Portion | Value |
| Q9GZK7 | Olfactory receptor | 1 . . . 308 | 147/308 (47%) | 4e−77 |
| | 11A1 (Hs6M1- | 1 . . . 306 | 202/308 (64%) | |
| | 18)—Homo | | | |
| | sapiens (Human), | | | |
| | 315 aa. | | | |
| O13036 | CHICK | 7 . . . 311 | 139/305 (45%) | 1e−76 |
| | OLFACTORY | 4 . . . 308 | 198/305 (64%) | |
| | RECEPTOR 7— | | | |
| | Gallus gallus | | | |
| | (Chicken), 323 aa. | | | |
| Q9JKA6 | OLFACTORY | 4 . . . 313 | 143/311 (45%) | 1e−75 |
| | RECEPTOR P2— | 1 . . . 311 | 194/311 (61%) | |
| | Mus musculus | | | |
| | (Mouse), 315 aa. | | | |
| Q9WU86 | ODORANT | 14 . . . 308 | 144/295 (48%) | 2e−75 |
| | RECEPTOR S1— | 21 . . . 315 | 189/295 (63%) | |
| | Mus musculus | | | |
| | (Mouse), 324 aa. | | | |
| Q9UGF6 | Olfactory receptor | 7 . . . 305 | 138/299 (46%) | 5e−75 |
| | 5V1 (Hs6M1-21)— | 4 . . . 302 | 199/299 (66%) | |
| | Homo sapiens | | | |
| | (Human), 321 aa. | | | |

PFam analysis predicts that the NOV65a protein contains the domains shown in the Table 65E.

TABLE 65E
Domain Analysis of NOV65a

| | | Identities/ | |
|---|---|---|---|
| | NOV65a | Similarities for | Expect |
| Pfam Domain | Match Region | the Matched Region | Value |
| granulin: domain | 144 . . . 155 | 7/13 (54%) | 1.7 |
| 1 of 1 | | 11/13 (85%) | |
| Trypan_glycop: | 218 . . . 241 | 6/24 (25%) | 7.9 |
| domain 1 of 1 | | 21/24 (88%) | |
| 7tm_1: domain | 44 . . . 293 | 53/268 (20%) | 1.5e−31 |
| 1 of 1 | | 172/268 (64%) | |

### Example 66

The NOV66 clone was analyzed, and the nucleotide and predicted polypeptide sequences are shown in Table 66A.

TABLE 66A
NOV66 Sequence Analysis

| | SEQ ID NO:197 | 987 bp |
|---|---|---|
| NOV66a, | CATCTTCCTATGTGTCATGTCTCCTCTTAATGACACAAAAATGGAAGTCCTTAGATTC | |
| CG59224-01 DNA Sequence | CTCCTTATCGGGATCACTGGACTGGAGAAAAGTCGCACCTGGATATCCATTCCTTTCT | |
| | TATCTGTGTACCTTCTTTCTTGGATGCCTAATTTTACCGTCCTCTTTTTTATCAAGAC | |
| | AGACCAAAGCCTCCATGAACCTATGTATTATTTGCTTTCCATGCTCTCCATCTCTGAC | |
| | CTACGGCTGTCTCTGTCTTCCTTACCCATCACTTTGGGACTATTCCTATTTGATGTCC | |
| | ATGAAATTCATGCAGCTCCATGCTTTGCCCAGGAATTTTTTATCCATCTGTTTACACT | |
| | CAGTGAAGCCTCTGTACTGTCTGTAATGGCATTTGACTGGTATGTGGCAATCCACAGT | |
| | CCTTTGAGATACAGCACTATCTTAACTAGTCCCAGAGCCATCAAAACAGGGGTTCTTC | |
| | TGACTTCCAAGAATGTTCTTTTGATCCTTCCACTGCCCTTTCTCTTGCAAAGGCTGAG | |
| | ATATTGTCATCAAAACCTGCTCTCCCACTCCTATTGTCTCCACCAGGATGTCATCAAG | |
| | CTGATGTGTTCTGACAACACAGTCAATGTTGTCTACGGACTCTGTGCAGGACTTTCTA | |
| | CTATGCTGGACTTGGTGTTTATTACCTTCTCCTATATGATTTTAAGGGCTGTACTGGG | |
| | AATTGCTACCCCCAGACAGCAGTTCAAGGCCCTCAACACGTGCATCTCTCACATCTCT | |
| | GGGATGTGTCTCCTATGATCCACGTCCTCATCGCTGATATTTTTCTGCTCGTCCCACC | |
| | GCTGTGCTTATCTTCTATGTGCCCACGCTGAGTGCTGCCATGCTCCACCAGTTTGCCA | |
| | CCTGTTGAATCCCATCGTGTACTGTGTGAAGACCCACCAAATCCGAGAAAAGGTTGTG | |
| | GGCAAACTTTGTCCAAAAGTAAGTTGATCAAAGGAATGAGAAAGGGAATGAATGTATA | |
| | A | |
| | ORF Start: ATG at 17 | ORF Stop: TGA at 953 |
| | SEQ ID NO:198 | 312 aa MW at 35250.7 kD |

| | |
|---|---|
| NOV66a, | MSPLNDTRMEVLRFLLIGITGLEKSRTWISIPFLSVYLLSWMGNFTVLFFIKTEQSLH |
| CG59224-01 Protein Sequence | EPMYYLLSMLSTSDLGLSLSSLPITLSLFLFDVHETHAAPCFAQEFFIHLFTVSEASV |
| | LSVMAFDWYVAIHSPLRYSTTLTSPRAIKTGVLLTSKNVLLILPLPFLLQRLRYCHQN |
| | LLSHSYCLHQDVMKLMCSDNTVNVVYGLCAGLSTMLOLVFITFSYMILRAVLGTATPR |
| | QQFKALNTCISHICAVLTFYVPTLSAAMLHQFARDVSPMTHVLMADIFLLVPPLLNPI |
| | VYCVKTHQIREKVVGKLCPKVS |

Further analysis of the NOV66a protein yielded the following properties shown in Table 66B.

TABLE 66B
Protein Sequence Properties NOV66a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.2007 probability |
| | located in mitochondrial inner membrane |
| SignalP | Likely cleavage site between residues 50 and 51 |
| analysis: | |

A search of the NOV66a protein against the Geneseq database, a proprietary database that contains sequences published in patents and patent publication, yielded several homologous proteins shown in Table 66C.

TABLE 66C
Geneseq Results for NOV66a

| | | NOV66a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72488 | Human OR-like | 1 . . . 312 | 309/313 (98%) | e−176 |
| | polypeptide query | 1 . . . 313 | 309/313 (98%) | |
| | sequence, SEQ ID | | | |
| | NO: 2169—Homo | | | |
| | sapiens, 319 aa. | | | |
| | [WO200127158-A2, | | | |
| | Apr. 19, 2001] | | | |
| AAG71557 | Human olfactory | 1 . . . 312 | 309/313 (98%) | e−176 |
| | receptor poly- | 1 . . . 313 | 309/313 (98%) | |
| | peptide, SEQ ID | | | |
| | NO: 1238—Homo | | | |
| | sapiens, 319 aa. | | | |
| | [WO200127158-A2, | | | |
| | Apr. 19, 2001] | | | |
| AAU24573 | Human olfactory | 1 . . . 310 | 186/311 (59%) | e−109 |
| | receptor | 1 . . . 311 | 246/311 (78%) | |
| | AOLFR63—Homo | | | |
| | sapiens, 313 aa. | | | |
| | [WO200168805-A2, | | | |
| | Sep. 20, 2001] | | | |
| AAG71558 | Human olfactory | 1 . . . 310 | 185/311 (59%) | e−108 |
| | receptor Homo | 1 . . . 311 | 245/311 (78%) | |
| | sapiens, 313 aa. | | | |
| | [WO200127158-A2, | | | |
| | Apr. 19, 2001] | | | |
| AAU24682 | Human olfactory | 1 . . . 307 | 188/308 (61%) | e−106 |
| | receptor | 1 . . . 306 | 237/308 (76%) | |
| | AOLFR181—Homo | | | |
| | sapiens, 312 aa. | | | |
| | [WO200168805-A2, | | | |
| | Sep. 20, 2001] | | | |

In a BLAST search of public sequence databases, the NOV66a protein was found to have homology to the proteins shown in the BLASTP data in Table 66D.

TABLE 66D
Public BLASTP Results for NOV66a

| | | NOV64a | Identities/ | |
|---|---|---|---|---|
| Protein | | Residues/ | Similarities for | |
| Accession | Protein | Match | the Matched | Expect |
| Number | Organism/Length | Residues | Portion | Value |
| AAL351091 | PROSTATE- | 14 . . . 304 | 141/293 (48%) | 2e−77 |
| | SPECIFIC G | 11 . . . 303 | 199/293 (67%) | |
| | PROTEIN- | | | |
| | COUPLED | | | |
| | RECEPTOR | | | |
| | RA1C—Mus | | | |
| | musculus | | | |
| | (Mouse), 320 aa. | | | |
| O88628 | Olfactory | 14 . . . 304 | 141/293 (48%) | 2e−77 |
| | receptor 51E2 | 11 . . . 303 | 200/293 (68%) | |
| | (G-protein | | | |
| | coupled receptor | | | |
| | RA1c)—Rattus | | | |
| | norvegicus | | | |
| | (Rat), 320 aa. | | | |
| CAC38935 | SEQUENCE 9 | 5 . . . 304 | 145/302 (48%) | 2e−77 |
| | FROM PATENT | 6 . . . 306 | 206/302 (68%) | |
| | WO0131014— | | | |
| | Homo sapiens | | | |
| | (Human), 318 aa. | | | |
| CAC37756 | SEQUENCE 1 | 5 . . . 304 | 145/302 (48%) | 3e−77 |
| | FROM PATENT | 5 . . . 305 | 206/302 (68%) | |
| | WO0125434— | | | |
| | Homo sapiens | | | |
| | (Human), 317 aa. | | | |
| Q9H255 | Olfactory receptor | 14 . . . 304 | 139/293 (47%) | 2e−76 |
| | 51E2 (Prostate | 11 . . . 303 | 198/293 (67%) | |
| | specific G-protein | | | |
| | coupled receptor) | | | |
| | (HPRAJ)— | | | |
| | Homo sapiens | | | |
| | (Human), 320 aa. | | | |

PFam analysis predicts that the NOV66a protein contains the domains shown in the Table 66E.

TABLE 66E
Domain Analysis of NOV66a

| | | Identities/ | |
|---|---|---|---|
| | NOV64a | Similarities for | Expect |
| Pfam Domain | Match Region | the Matched Region | Value |
| 7tm_1: domain | 43 . . . 151 | 30/111 (27%) | 6.3e−14 |
| 1 of 2 | | 73/111 (66%) | |
| 7tm_1: domain | 43 . . . 151 | 16/92 (17%) | 0.052 |
| 1 of 2 | | 52/92 (57%) | |

### Example 67

The NOV67 clone was analyzed, and the nucleotide and predicted polypeptide sequences are shown in Table 67A.

TABLE 67A
NOV67 Sequence Analysis

| | SEQ ID NO:199 | 994 bp | |
|---|---|---|---|
| NOV67a, | CACAATGTCTGTCTTCAATAGTTCTCCCTTATACCCTCCCTTCCTCCTAACGGGCCTC | | |
| CG59222-01 DNA Sequence | | | |
| | TCAGGCCTTGAAAGCAGATATGACTTGATTTCCCTGCCCATCTTCTTGGTTTATGCCA | | |
| | CCTCAATTGCCGGGAACATTAGCATCCTCTTCATTATCAGAACTGAGTCTTCCCTCCA | | |
| | CCAACCGATGTATTACTTTCTGTCAATGCTGGCATTCACTGACCTGGGCCTATCTAAC | | |
| | ACTACCTTACCTACCATGTTCAGTGTCTTCTGGTTCCATGCCCGCGAGATCTCCTTCA | | |
| | ATGCTTCTCTGGTCCAAATGTACTTCATTCATCTTTTCTCGATTATTGAGTCAGCTGT | | |
| | ACTCCTGGCTATGGCCTTTGACTGCTTTATAGCAATCTGTGAACCCTTGCGCTATGCA | | |
| | GCCATCCTAACCAATGATGTAATCATTGGGATTGGGTTGGCAATTGCTGGAAGGGCCT | | |
| | TGGCTCTGGTCTTTCCAGCTTCTTTCCTCTTGAAGAGGCTTCAATATCATGATGTCAA | | |
| | TATTCTGTCCTACCTCTTCTGCCTGCACCAGGACCTCATAAAGACGACTGTATCCAAC | | |
| | TGTCGAGTCAGCAGCATCTATGGCCTCATGGTGGTCATCTGTTCCATGGGACTTGATT | | |
| | CAGTCCTTCTCCTCCTCTCCTATGTCCTCATCCTCCCCACACTGTTCAGTATAGCCTC | | |
| | CAAGGCAGAGAGAGTGAGAGCCCTCAATACTTGCATCTCCCACATCTGTGCTGTACTC | | |
| | ACCTTCTATACACCAATGATTCGCCTATCTATGATCCATCGCTATCCACAGAATCCTT | | |
| | CCTCAATTGTCCATGTGCTGATGGCCAATGTCTACTTGCTGGTTCCACCTCTCATGAA | | |
| | CCCCGTTGTCTACAGTGTTAAGACCAAGCAGATTCGTGACAGAATCTTCAATAAATTC | | |
| | AAGAAACATGAAGTGTAGATCACAGAGATTCTCAAACATAACTTTCCCTCCATTCCCC | | |
| | ATATATTT | | |
| | ORF Start: ATG at 5 | | ORF Stop: TAG at 944 |
| | SEQ ID NO:200 | 313 aa | MW at 35044.2 kD |

| | |
|---|---|
| NOV67a, | MSVFNSSALYPRFLLTGLSGLESRYDLISLPIFLVYATSIAGNISILFIIRTESSLHQ |
| CG59222-01 Protein Sequence | |
| | PMYYFLSMLAFTDLGLSNTTLPTMFSVFWFHAREISFNACLVQMYFIHVFSIIESAVL |
| | LAMAFDCFIAICEPLRYAAILTNDVIIGIGLAIAGRALALVFPASFLLKRLQYHDVNI |
| | LSYLFCLHQDLIKTTVSNCRVSSIYGLMVVICSMGLDSVLLLLSYVLILGTVLSIASK |
| | AERVRALNTCISHICAVLTPYTPMTGLSMIHRYGQNASSIVHVLMANVYLLVPPLMNP |
| | VVYSVKTKQIRDRIFNKFKKHEV |

Further analysis of the NOV67a protein yielded the following properties shown in Table 67B.

TABLE 67B
Protein Sequence Properties NOV67a

| | |
|---|---|
| PSort | 0.6000 probability located in plasma membrane; 0.4047 |
| analysis: | probability located in mitochondrial inner membrane; 0.4000 |
| | probability located in Golgi body; 0.3480 probability located |
| | in mitochondrial intermembrane space |
| SignalP | No Known Signal Sequence Predicted |
| analysis: | |

A search of the NOV67a protein against the Geneseq database, a proprietary database that contains sequences published in patents and patent publication, yielded several homologous proteins shown in Table 67C.

TABLE 67C
Geneseq Results for NOV67a

| | | NOV67a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72605 | Human OR-like | 1 . . . 309 | 295/310 (95%) | e−163 |
| | polypeptide query | 4 . . . 313 | 298/310 (95%) | |
| | sequence, SEQ ID | | | |
| | NO: 2286—Homo | | | |
| | sapiens, 318 aa. | | | |
| | [WO200127158-A2, | | | |
| | Apr. 19, 2001] | | | |
| AAG71519 | Human olfactory | 1 . . . 309 | 295/310 (95%) | e−163 |
| | receptor poly- | 4 . . . 313 | 298/310 (95%) | |
| | peptide, SEQ ID | | | |
| | NO: 1200—Homo | | | |
| | sapiens, 318 aa. | | | |
| | [WO200127158-A2, | | | |
| | Apr. 19, 2001] | | | |
| AAU24683 | Human olfactory | 5 . . . 308 | 178/304 (58%) | e−102 |
| | receptor | 9 . . . 312 | 235/304 (76%) | |
| | AOLFR182—Homo | | | |
| | sapiens, 314 aa. | | | |
| | [WO200168805-A2, | | | |
| | Sep. 20, 2001] | | | |
| AAG71715 | Human olfactory | 5 . . . 308 | 178/304 (58%) | e−102 |
| | receptor poly- | 9 . . . 312 | 235/304 (76%) | |
| | peptide, SEQ ID | | | |
| | NO: 1396—Homo | | | |
| | sapiens, 314 aa. | | | |
| | [WO200127158-A2, | | | |
| | Apr. 19, 2001] | | | |
| ABB44526 | Human GPCR4a | 5 . . . 308 | 169/304 (55%) | 2e−96 |
| | polypeptide SEQ ID | 6 . . . 309 | 227/304 (74%) | |
| | NO 11—Homo | | | |
| | sapiens, 315 aa. | | | |
| | [WO200174904-A2, | | | |
| | Oct. 11, 2001] | | | |

In a BLAST search of public sequence databases, the NOV67a protein was found to have homology to the proteins shown in the BLASTP data in Table 67D.

TABLE 67D
Public BLASTP Results for NOV67a

| | | NOV67a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9H344 | Olfactory | 13 . . . 308 | 154/296 (52%) | 2e−91 |
| | receptor 51I2 | 12 . . . 307 | 221/296 (74%) | |
| | (HOR5′beta12)— | | | |
| | Homo sapiens | | | |
| | (Human), 312 aa. | | | |
| Q9H2C8 | ODORANT | 2 . . . 308 | 160/307 (52%) | 5e−89 |
| | RECEPTOR | 10 . . . 316 | 216/307 (70%) | |
| | HOR3′BETA1— | | | |
| | Homo sapiens | | | |
| | (Human), 321 aa. | | | |
| Q9H343 | Olfactory | 5 . . . 312 | 156/309 (50%) | 9e−89 |
| | receptor 51I1 | 5 . . . 313 | 223/309 (71%) | |
| | (HOR5′beta11)— | | | |
| | Homo sapiens | | | |
| | (Human), 314 aa. | | | |
| AAL35109 | PROSTATE- | 13 . . . 309 | 148/297 (49%) | 2e−86 |
| | SPECIFIC G | 11 . . . 307 | 207/297 (68%) | |
| | PROTEIN- | | | |
| | COUPLED | | | |
| | RECEPTOR | | | |
| | RA1C—Mus | | | |
| | musculus | | | |
| | (Mouse), 320 aa. | | | |
| Q924X8 | OLFACTORY | 2 . . . 304 | 150/303 (49%) | 1e−85 |
| | RECEPTOR | 3 . . . 305 | 221/303 (72%) | |
| | S85—Mus | | | |
| | musculus | | | |
| | (Mouse), 314 aa. | | | |

PFam analysis predicts that the NOV67a protein contains the domains shown in the Table 67E.

TABLE 67E
Domain Analysis of NOV67a

| | | Identities/ | |
|---|---|---|---|
| | NOV67a | Similarities for | Expect |
| Pfam Domain | Match Region | the Matched Region | Value |
| 7tm_1: domain | 42 . . . 138 | 24/99 (24%) | 7.8e−14 |
| 1 of 1 | | 67/99 (68%) | |

### Example 68

The NOV68 clone was analyzed, and the nucleotide and predicted polypeptide sequences are shown in Table 68A.

TABLE 68A
NOV68 Sequence Analysis

| | SEQ ID NO:201 | 981 bp | |
|---|---|---|---|
| NOV68a, | GCAATGAGAAACCGCAGTGTTGTCCCTGAGTTTGTCCTCCTCGGGCTGTCAGCTGGCC | | |
| CG59220-01 DNA Sequence | | | |
| | CCCACACCCACACTCTGCTCTTTGTGCTGTTCGTGGTGATTTGCCTCCTGACTGTGAT | | |
| | GGGAAACCTGCTGCTGCTGGTGGTGATTAATGCTGATTCTTGCCTCCACACACCCATG | | |
| | TACTTCTTCCTGGGACAATTGTCCTTCTTGGATCTCTGCCATTCCTCTGTCACTGCAC | | |
| | CTAAGCTGTTGGAGAACCTCCTGTCTGAGAAGAAAACCATCTCAGTAGAGGGCTGCAT | | |
| | GGCTCAGGTCTTCTTTGTGTTTGCCACTGGGGGCACTGAATCCTCCCTGCTTGCTGTG | | |
| | ATGGCCTATGACCGCTATGTTGCCATCAGCTCTCCTTTGCTCTATGCCCAACTGATGA | | |
| | ACAGACAGCTGTGTTCAGGGCTGGTGGGGGGCTCATGGGGCTTGGCTTTTCTGGATGC | | |
| | CCTCATCAATATCCTTGTAGCTCTCAATTTAGACTTCTGTGACGCTCAAAATATCCAC | | |
| | CACTTCAGCTGTGAGCTGCCCTCTCTCTATCCTTTGTCTTGCTCTGATGTGTCAGCAA | | |
| | GTTTTACCACCCTGCTCTGCTCCAGCTTCCTGCATTTCTTTGGAAATTTTCTCATGAT | | |
| | ATTCTTGTCTTATATTTGCATTTTGTCCACCATCCTGAGGATCAGCTCCACTACAGGC | | |
| | ACAAGCAAAGCCTTCTCCACCTGCTCCTCCCACCTCACTGCACTGATTTTCTTTTATC | | |
| | GCTCCGGATTACTCCGCTATCTCATGCCAAATTCAGGATCCATTCAAGAGCTGATCTT | | |
| | CTCCTTGCAGTACAGCGTGATCACTCCCATGCTGAATCTCCTCATTTACAGCCTGAAG | | |
| | AACAGGGAGGTGAAGGCAGCTGTGAGAAGAACATTGAGAAAATATTTCTAGTGTTTCA | | |
| | ATAGACTTATGAAATCAGAATGATGAGGGAACTGGATAGAACTGCAACAAGCA | | |
| | ORF Start: ATG at 4 | | ORF Stop: TAG at 919 |
| | SEQ ID NO:202 | 305 aa | MW at 33732.3 kD |

| | |
|---|---|
| NOV68a | MRNRSVVPEFVLLGLSAGPQTQTLLFVLFVVICLLTVMGNLLLLVVINADSCLHTPMY |
| CG59220-01 Protein Sequence | |
| | FFLGQLSFLDLCHSSVTAPKLLENLLSEKKTISVEGCMAQVFFVFATGGTESSLLAVM |
| | AYDRYVAISSPLLYGQVMNRQLCSGLVGGSWGLAFLDALINILVALNLDFCEAQNTGR |
| | FSCELPSLYPLSCSDVSASFTTLLCSSFLHFFGNFLMTFLSYICTLSTILRISSTTGR |
| | SKAFSTCSSHLTAVIFFYGSGLLRYLMPNSGSIQELIESLQYSVITPMLNLLTYSLKN |
| | REVKAAVRRTLRKYF |

Further analysis of the NOV68a protein yielded the following properties shown in Table 68B.

TABLE 68B
Protein Sequence Properties NOV68a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | Likely cleavage site between residues 50 and 51 |
| analysis: | |

A search of the NOV68a protein against the Geneseq database, a proprietary database that contains sequences published in patents and patent publication, yielded several homologous proteins shown in Table 68C.

TABLE 68C
Geneseq Results for NOV68a

| | | | | |
|---|---|---|---|---|
| | | NOV68a | Identities/ | |
| | Protein/ | Residues/ | Similarities for | |
| Geneseq | Organism/Length | Match | the Matched | Expect |
| Identifier | [Patent #, Date] | Residues | Region | Value |

| | | | | |
|---|---|---|---|---|
| AAU24771 | Human olfactory | 3 . . . 304 | 212/302 (70%) | e−120 |
| | receptor | 5 . . . 306 | 251/302 (82%) | |
| | AOLFR328— | | | |
| | Homo sapiens, | | | |
| | 312 aa. | | | |
| | [WO200168805- | | | |
| | A2, Sep. 20, | | | |
| | 2001] | | | |
| AAG98585 | Mouse olfactory | 66 . . . 279 | 144/214 (67%) | 1e−78 |
| | receptor 7— | 1 . . . 214 | 169/214 (78%) | |
| | Mus musculus | | | |
| | domesticus, | | | |
| | 214 aa. | | | |
| | [WO200146262- | | | |
| | A2, Jun. 28, | | | |
| | 2001] | | | |
| AAG72680 | Murine OR-like | 3 . . . 305 | 148/305 (48%) | 3e−74 |
| | polypeptide query | 20 . . . 324 | 201/305 (65%) | |
| | sequence, SEQ | | | |
| | ID NO: 2362— | | | |
| | Mus musculus, | | | |
| | 337 aa. | | | |
| | [WO200127158- | | | |
| | A2, Apr. 19, | | | |
| | 2001] | | | |
| AAG71546 | Human olfactory | 3 . . . 301 | 143/302 (47%) | 2e−73 |
| | receptor poly- | 5 . . . 306 | 201/302 (66%) | |
| | peptide, SEQ ID | | | |
| | NO: 1227— | | | |
| | Homo sapiens, | | | |
| | 315 aa. | | | |
| | [WO200127158- | | | |
| | A2, Apr. 19, | | | |
| | 2001] | | | |
| AAG66701 | Human GPCR1 | 3 . . . 301 | 143/302 (47%) | 2e−73 |
| | polypeptide— | 5 . . . 306 | 201/302 (66%) | |
| | Homo sapiens, | | | |
| | 311 aa. | | | |
| | [WO200160865- | | | |
| | A2, Aug. 23, | | | |
| | 2001] | | | |

In a BLAST search of public sequence databases, the NOV68a protein was found to have homology to the proteins shown in the BLASTP data in Table 68D.

TABLE 68D
Public BLASTP Results for NOV68a

| | | NOV68a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9JM36 | OLFACTORY | 66 . . . 279 | 144/214 (67%) | 5e−78 |
| | RECEPTOR—Mus | 1 . . . 214 | 169/214 (78%) | |
| | musculus | | | |
| | domesticus | | | |
| | (western European | | | |
| | house mouse), 214 | | | |
| | aa (fragment). | | | |
| Q9QZ18 | OLFACTORY | 3 . . . 299 | 142/299 (47%) | 2e−72 |
| | RECEPTOR—Mus | 5 . . . 303 | 193/299 (64%) | |
| | musculus (Mouse), | | | |
| | 312 aa. | | | |
| Q9EPG6 | B1 OLFACTORY | 3 . . . 299 | 140/299 (46%) | 2e−72 |
| | RECEPTOR—Mus | 5 . . . 303 | 196/299 (64%) | |
| | musculus (Mouse), | | | |
| | 314 aa. | | | |
| P23266 | Olfactory receptor- | 3 . . . 305 | 142/305 (46%) | 9e−72 |
| | like protein F5— | 5 . . . 309 | 196/305 (63%) | |
| | Rattus norvegicus | | | |
| | (Rat), 313 aa. | | | |
| Q9EQA3 | ODORANT | 3 . . . 305 | 143/306 (46%) | 2e−71 |
| | RECEPTOR K30— | 5 . . . 310 | 202/306 (65%) | |
| | Mus musculus | | | |
| | (Mouse), 311 aa. | | | |

PFam analysis predicts that the NOV68a protein contains the domains shown in the Table 68E.

TABLE 68E
Domain Analysis of NOV68a

| | | Identities/ | |
|---|---|---|---|
| | NOV68a | Similarities for | Expect |
| Pfam Domain | Match Region | the Matched Region | Value |
| 7tm_1: domain | 39 . . . 286 | 54/268 (20%) | 1.7e−29 |
| 1 of 1 | | 169/268 (63%) | |

### Example 69

The NOV69 clone was analyzed, and the nucleotide and predicted polypeptide sequences are shown in Table 69A.

TABLE 69A
NOV69 Sequence Analysis

| | SEQ ID NO:203 | 957 bp | |
|---|---|---|---|
| NOV69a, | GTCCACAATGGCCAATCAGACTGTGGTGACTGAGTTCTTCCTCCAAGGCCTGACGGAT | | |
| CG59218-01 DNA Sequence | | | |
| | ACCAAAGAGCTTCAGGTGGCTGTTTTTCTGCTCCTGCTGCTTGCCTACCTTGTGACTG | | |
| | TCTCTGGGAACCTGATCATCATCAGCCTGACCTTGCTGGACACCCGCCTGCAGACATC | | |
| | TATGTACTTATTTCTCCAGAATCTGTCCTGCTTAGAAATTTGGTTCCAGACAGTCATC | | |
| | GTGCCCAAGATGCTGCTCAACATTGCCATGGGGACCAAGACCGTTAGCTTTGCTGGGT | | |
| | GCATTACCCAGGACTTTTTCTTTCCACATCTTCTGGGGGCCACAGAGTTCTTCCTCCT | | |
| | CACAGCCATGGCCTATGACCAGTATATTGCCATCTGCAAGCCCCTCCACTACCCCATG | | |
| | CTCATAAGTAGTAGAGTCTGCACACAGCTCATCCTCACCTGCTGGCTACTAGGTTTCT | | |
| | CCTTCATCATCATGCCTGTCATCCTGACCAGTCAGCTTCCATTCTGTGATACCCACAT | | |
| | CAAGCATTTCTTCTGTGACTACACGCCTCTAATGGAGGTGGTCTGCAGTGGGCCAAAG | | |
| | GTGCTGGAGATGGTGGATTTTACCCTGGCCTTAGTAGCACTGTTTGGCACCTTGGTAC | | |
| | TCATCACCCTGTCCTATGTCCAGATCATCCAGACAATTGTCACAATCCCCGCTGTCCA | | |
| | GGAGAGGAAGAAGGCTTTCTCTACCTGTTCCTCTCATGTCATTATGGTTACCATGTGT | | |
| | TATGACAGCTCCTTCTTTATGTATGTCAAGCCCTCTCCAGGAAACTGGGTTGATGTCA | | |
| | ACAAGGGAGTCTCTCTAATCAATACAATTATTGCCCCACTGTTAAATCCCTTCATCTC | | |
| | TACTCTGAGGAACCAACAAGTTAAGCAGGTAATGAAAGACCTAGTCAGAAAAATGACT | | |
| | TTGTTCCAAAATAAATAAGCGCCCTAAAA | | |
| | ORF Start: ATG at 8 | | ORF Stop: TAA at 944 |
| | SEQ ID NO:204 | 312 aa | MW at 35358.1 kD |

| | |
|---|---|
| NOV69a, | MANQTVVTEFFLQGLTDTKELQVAVFLLLLLAYLVTVSGNLIIISLTLLDTRLQTSMY |
| CG59218-01 Protein Sequence | |
| | LFLQNLSCLEIWFQTVIVPKMLLNIAMGTKTVSFAGCITQDFFFPHLLGATEFFLLTA |
| | MAYDQYIAICRPLHYPMLISSRVCTQLILTCWLLGFSFTIMPVTLTSQLPFCDTHIKH |
| | FFCDYTPLMEVVCSGPKVLEMVDFTLALVALFGTLVLITLSYVQIIQTIVRIPAVQER |
| | KKAFSTCSSHVIMVTMCYDSCFFMYVKPSPGKWVDVNKGVSLINTIIAPLLNPFICTL |
| | RNQQVKQVMKDLVRKNTLFQNK |

Further analysis of the NOV69a protein yielded the following properties shown in Table 69B.

TABLE 69B
Protein Sequence Properties NOV69a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.0300 probability |
| | located in mitochondrial inner membrane |
| SignalP | Likely cleavage site between residues 40 and 41 |
| analysis: | |

A search of the NOV69a protein against the Geneseq database, a proprietary database that contains sequences published in patents and patent publication, yielded several homologous proteins shown in Table 69C.

TABLE 69C
Geneseq Results for NOV69a

| | | NOV69a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72538 | Human OR-like | 1 . . . 312 | 284/317 (89%) | e−157 |
| | polypeptide query | 1 . . . 313 | 293/317 (91%) | |
| | sequence, SEQ ID | | | |
| | NO: 2219—Homo | | | |
| | sapiens, 313 aa. | | | |
| | [WO200127158-A2, | | | |
| | Apr. 19, 2001] | | | |
| AAG72229 | Human olfactory | 1 . . . 312 | 284/317 (89%) | e−157 |
| | receptor poly- | 1 . . . 313 | 293/317 (91%) | |
| | peptide, SEQ ID | | | |
| | NO:1910—Homo | | | |
| | sapiens, 313 aa. | | | |
| | [WO200127158-A2, | | | |
| | Apr. 19, 2001] | | | |
| AAU24761 | Human olfactory | 1 . . . 306 | 173/307 (56%) | 2e−96 |
| | receptor | 1 . . . 306 | 227/307 (73%) | |
| | AOLFR112B— | | | |
| | Homo sapiens, | | | |
| | 309 2001] | | | |
| AAU24765 | Human olfactory | 1 . . . 306 | 166/307 (54%) | 2e−94 |
| | receptor | 1 . . . 306 | 227/307 (73%) | |
| | AOLFR225B— | | | |
| | Homo sapiens, | | | |
| | 309 aa. | | | |
| | [WO200168805-A2, | | | |
| | Sep. 20, 2001] | | | |
| AAG66353 | GPCR partial | 1 . . . 309 | 160/311 (51%) | 4e−87 |
| | protein sequence— | 1 . . . 310 | 209/311 (66%) | |
| | Unidentified, | | | |
| | 313 aa. | | | |
| | [WO200155179-A2, | | | |
| | Aug. 2, 2001] | | | |

In a BLAST search of public sequence databases, the NOV69a protein was found to have homology to the proteins shown in the BLASTP data in Table 69D.

TABLE 69D
Public BLASTP Results for NOV69a

| | | NOV69a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9Z1V0 | OLFACTORY | 1 . . . 309 | 160/311 (51%) | 2e−86 |
| | RECEPTOR | 1 . . . 310 | 209/311 (66%) | |
| | C6—Mus | | | |
| | musculus | | | |
| | (Mouse), 313 aa. | | | |
| CAC88326 | SEQUENCE 18 | 8 . . . 306 | 142/301 (47%) | 4e−78 |
| | FROM PATENT | 12 . . . 311 | 200/301 (66%) | |
| | WO0164879— | | | |
| | Homo sapiens | | | |
| | (Human), 331 aa. | | | |
| CAC88328 | SEQUENCE 22 | 8 . . . 306 | 142/301 (47%) | 2e−77 |
| | FROM PATENT | 12 . . . 311 | 198/301 (65%) | |
| | WO0164879— | | | |
| | Homo sapiens | | | |
| | (Human), 331 aa. | | | |
| CAC88327 | SEQUENCE 20 | 8 . . . 306 | 141/301 (46%) | 8e−77 |
| | FROM PATENT | 12 . . . 311 | 198/301 (64%) | |
| | WO0164879— | | | |
| | Homo sapiens | | | |
| | (Human), 331 aa. | | | |
| O70270 | OLFACTORY | 3 . . . 308 | 136/307 (44%) | 4e−76 |
| | RECEPTOR- | 11 . . . 316 | 208/307 (67%) | |
| | LIKE | | | |
| | PROTEIN— | | | |
| | Rattus | | | |
| | norvegicus (Rat), | | | |
| | 327 aa. | | | |

PFam analysis predicts that the NOV69a protein contains the domains shown in the Table 69E.

TABLE 69E
Domain Analysis of NOV69a

| | | Identities/ | |
|---|---|---|---|
| | NOV69a | Similarities for | Expect |
| Pfam Domain | Match Region | the Matched Region | Value |
| 7tm_1: domain | 39 . . . 244 | 47/214 (22%) | 1.9e−25 |
| 1 of 1 | | 147/214 (69%) | |

### Example 70

The NOV70 clone was analyzed, and the nucleotide and predicted polypeptide sequences are shown in Table 70A.

TABLE 70A
NOV70 Sequence Analysis

| | SEQ ID NO:205 | 962 bp | |
|---|---|---|---|
| NOV70a, | CATCTTCCTATGTGTCATGTCTCCTCTTAATGACACAAAAATGGAAGTCCTTAGATTC | | |
| CG59216-01 DNA Sequence | | | |
| | CTCCTTATCGGGATCACTGGACTGCAGAAAAGTCGCACCTGGATATCCATTCCTTTCT | | |
| | TATCTGTGTACCTTCTTTCTTCGATCGGTAATTTTACCGTCCTCTTTTTTATCAAGAC | | |
| | AGACCAAAGCCTCCATCAACCTATGTATTATTTGCTTTCCATGCTCTCCATCTCTGAC | | |
| | CTAGGGCTGTCTCTGTCTTCCTTACCCATCACTTTGGGACTATTCCTATTTGATGTCC | | |
| | ATGAAATTCATGCAGCTCCATGCTTTGCCCAGGAATTTTTTATCCATCTGTTTACAGT | | |
| | CAGTGAAGCCTCTGTACTGTCTGTAATGGCATTTGACTGGTATGTGGCAATCCACAGT | | |
| | CCTTTGAGATACAGCACTATCTTAACTAGTCCCAGAGCCATCAAAACAGGGGTTCTTC | | |
| | TGACTTCCAAGAATGTTCTTTTGATCCTTCCACTGCCCTTTCTCTTGCAAAGGCTGAG | | |
| | ATATTGTCATCAAAACCTGCTCTCCCACTCCTATTGTCTCCACCAGGATGTCATGAAG | | |
| | CTGATGTGTTCTGACAACACAGTCAATGTTGTCTACGGACTCTGTGCAGGACTTTCTA | | |
| | CTATGCTCCACTTGGTGTTTATTACCTTCTCCTATATTATGATTTTAAGGGCTGTACT | | |
| | GGGAATTGCTACCCCCAGACAGCAGTTCAAGCCCCTCAACACCTCCATCTCTCACATC | | |
| | TGTGCTCTCCTTATCTTCTATGTGCCCACGCTGAGTGCTCCCATGCTCCACCAGTTTC | | |
| | CCAGGGATGTGTCTCCTATGATCCACGTCCTCATGGCTGATATTTTTCTGCTGGTGCC | | |
| | ACCCCTGTTGAATCCCATCGTGTACTGTGTGAAGACCCACCAAATCCGAGAAAAGGTT | | |
| | GTGGGGAAACTTTGTCCAAAAGTAAGTTGATCAA | | |
| | ORF Start: ATG at 17 | | ORF Stop: TGA at 956 |
| | SEQ ID NO:206 | 313 aa | MW at 35363.9 kD |

| | |
|---|---|
| NOV70a, | MSPLNDTKMEVLRFLLIGITGLEKSRTWISIPFLSVYLLSWMGNFTVLFFIKTEQSLH |
| CG59216-01 Protein Sequence | |
| | EPMYYLLSMLSISDLGLSLSSLPITLGLFLFDVHEIHAAPCFAQEFFIHLFTVSEASV |
| | LSVMAFDWYVAIHSPLRYSTILTSPRAIKTGVLLTSKNVLLILPLPFLLQRLRYCHQN |
| | LLSHSYCLEQDVMKLMCSDNTVNVVYGLCAGLSTMLDLVFTTFSYTMILRAVLGIATP |
| | RQQFKALNTCISHICAVLIFYVPTLSAAMLHQFARDVSPMTHVLMADIFLLVPPLLNP |
| | IVYCVKTHQIREKVVCKLCPKVS |

Further analysis of the NOV70a protein yielded the following properties shown in Table 70B.

TABLE 70B
Protein Sequence Properties NOV70a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.2007 probability |
| | located in mitochondrial inner membrane |
| SignalP | Likely cleavage site between residues 50 and 51 |
| analysis: | |

A search of the NOV70a protein against the Geneseq database, a proprietary database that contains sequences published in patents and patent publication, yielded several homologous proteins shown in Table 70C.

TABLE 70C
Geneseq Results for NOV70a

| | | NOV70a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAG72488 | Human OR-like | 1 . . . 313 | 310/313 (99%) | e−178 |
| | polypeptide query | 1 . . . 313 | 310/313 (99%) | |
| | sequence, SEQ ID | | | |
| | NO: 2169—Homo | | | |
| | sapiens, 319 aa. | | | |
| | [WO200127158-A2, | | | |
| | Apr. 19, 2001] | | | |
| AAG71557 | Human olfactory | 1 . . . 313 | 310/313 (99%) | e−178 |
| | receptor poly- | 1 . . . 313 | 310/313 (99%) | |
| | peptide, SEQ ID | | | |
| | NO: 1238—Homo | | | |
| | sapiens, 319 aa. | | | |
| | [WO200127158-A2, | | | |
| | Apr. 19, 2001] | | | |
| AAU24573 | Human olfactory | 1 . . . 311 | 186/311 (59%) | e−110 |
| | receptor | 1 . . . 311 | 246/311 (78%) | |
| | AOLFR63—Homo | | | |
| | sapiens, 313 aa. | | | |
| | [WO200168805-A2, | | | |
| | Sep. 20, 2001] | | | |
| AAG71558 | Human olfactory | 1 . . . 311 | 185/311 (59%) | e−109 |
| | receptor poly- | 1 . . . 311 | 245/311 (78%) | |
| | peptide, SEQ ID | | | |
| | NO: 1239—Homo | | | |
| | sapiens, 313 aa. | | | |
| | [WO200127158-A2, | | | |
| | Apr. 19, 2001] | | | |
| AAU24682 | Human olfactory | 1 . . . 308 | 188/308 (61%) | e−107 |
| | receptor | 1 . . . 306 | 238/308 (77%) | |
| | AOLFR181 — | | | |
| | Homo sapiens, | | | |
| | 312 aa. | | | |
| | [WO200168805-A2, | | | |
| | Sep. 20, 2001] | | | |

In a BLAST search of public sequence databases, the NOV70a protein was found to have homology to the proteins shown in the BLASTP data in Table 70D.

TABLE 70D
Public BLASTP Results for NOV70a

| | | NOV70a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| CAC38935 | SEQUENCE 9 | 5 . . . 305 | 145/302 (48%) | 5e−79 |
| | FROM PATENT | 6 . . . 306 | 207/302 (68%) | |
| | WO0131014— | | | |
| | Homo sapiens | | | |
| | 318 aa. | | | |
| AAL35109 | PROSTATE- | 14 . . . 305 | 141/293 (48%) | 7e−79 |
| | SPECIFIC G | 11 . . . 303 | 199/293 (67%) | |
| | PROTEIN- | | | |
| | COUPLED | | | |
| | RECEPTOR | | | |
| | RA1C—Mus | | | |
| | musculus | | | |
| | (Mouse), 320 aa. | | | |
| CAC37756 | SEQUENCE 1 | 5 . . . 305 | 145/302 (48%) | 7e−79 |
| | FROM PATENT | 5 . . . 305 | 207/302 (68%) | |
| | WO0125434— | | | |
| | Homo sapiens | | | |
| | (Human), 317 aa. | | | |
| O88628 | Olfactory receptor | 14 . . . 305 | 141/293 (48%) | 7e−79 |
| | 51E2 (G-protein | 11 . . . 303 | 200/293 (68%) | |
| | coupled receptor | | | |
| | RA1c)—Rattus | | | |
| | norvegicus | | | |
| | (Rat), 320 aa. | | | |
| Q9H255 | Olfactory receptor | 14 . . . 305 | 139/293 (47%) | 7e−78 |
| | 51E2 (Prostate | 11 . . . 303 | 198/293 (67%) | |
| | specific G-protein | | | |
| | coupled receptor) | | | |
| | (HPRAJ)—Homo | | | |
| | sapiens | | | |
| | (Human), 320 aa. | | | |

PFam analysis predicts that the NOV70a protein contains the domains shown in the Table 70E.

TABLE 70E
Domain Analysis of NOV70a

| | | Identities/ | |
|---|---|---|---|
| | NOV70a | Similarities for | Expect |
| Pfam Domain | Match Region | the Matched Region | Value |
| 7tm_1: domain | 43 . . . 151 | 30/111 (27%) | 6.3e−14 |
| 1 of 2 | | 73/111 (66%) | |
| YCF9: domain | 208 . . . 262 | 10/59 (17%) | 7.5 |
| 1 of 1 | | 31/59 (53%) | |
| 7tm_1: domain | 212 . . . 293 | 18/93 (19%) | 0.00034 |
| 2 of 2 | | 55/93 (59%) | |

### Example 71

The NOV71 clone was analyzed, and the nucleotide and predicted polypeptide sequences are shown in Table 71A.

TABLE 71A
NOV71 Sequence Analysis

| | SEQ ID NO:207 | 995 bp | |
|---|---|---|---|
| NOV71a, | GCACAATGTCTGTCTTCAATAGTTCTGCCTTATACCCTCGCTTCCTCCTAACGGGCCT | | |
| CG59214-01 DNA Sequence | | | |
| | CTCAGGCCTTGAAAGCAGATATGACTTGATTTCCCTGCCCATCTTCTTGGTTTATGCC | | |
| | ACCTCAATTGCCGGGAACATTAGCATCCTCTTCATTATCAGAACTGAGTCTTCCCTCC | | |
| | ACCAACCGATGTATTACTTTCTGTCAATGCTGGCATTCACTGACCTGGGCCTATCTAA | | |
| | CACTACCTTACCTACCATGTTCAGTGTCTTCTGGTTCCATGCCCGGGAGATCTCCTTC | | |
| | AATGCTTGTCTGGTCCAAATGTACTTCATTCATGTTTTCTCGATTATTGAGTCAGCTG | | |
| | TACTCCTGGCTATCGCCTTTGACTGCTTTATAGCAATCTGTGAACCCTTGCGCTATGC | | |
| | AGCCATCCTAACCAATGATGTAATCATTGGGATTGCGTTGGCAATTGCTGGAAGGGCC | | |
| | TTGGCTCTGGTCTTTCCAGCTTCTTTCCTCTTGAAGAGGCTTCAATATCATGATGTCA | | |
| | ATATTCTGTCCTACCTCTTCTGCCTGCACCAGGACCTCATAAAGACGACTGTATCCAA | | |
| | CTGTCGAGTCAGCAGCATCTATGGCCTCATGGTGGTCATCTGTTCCATGGGACTTGAT | | |
| | TCAGTGCTTCTCCTCCTCTCCTATGTCCTCATCCTGGGCACAGTGTTGAGTATAGCCT | | |
| | CCAAGGCAGAGAGAGTGAGAGCCCTCAATACTTGCATCTCCCACATCTGTGCTGTACT | | |
| | TCCTCAATTGTCCATGTGCTGATGGCCAATGTCTACTTGCTGGTTCCACCTCTCATGA | | |
| | CACCTTCTATACACCAATGATTGGGCTATCTATGATCCATCGCTATGGACASAATGCT | | |
| | ACCCCGTTGTCTACAGTGTTAAGACCAAGCAGATTCGTGACAGAATCTTCAATAAATT | | |
| | CAAGAAACATGAAGTGTAGATGACAGAGATTCTGAAACATAACTTTCCCTCCATTCCC | | |
| | CATATATTT | | |
| | ORF Start: ATG at 6 | | ORF Stop: TAG at 945 |
| | SEQ ID NO:208 | 313 aa | MW at 35044.2 kD |

| | |
|---|---|
| NOV71a, | MSVFNSSALYPRFLLTGLSGLESRYDLISLPTELVYATSIAGNTSILFTTRTESSLHQ |
| CG59214-01 Protein Sequence | |
| | PMYYFLSMLAFTDLGLSNTTLPTMFSVFWFHAREISFNACLVQMYFIHVFSIIESAVL |
| | LAMAFDCFIAICEPLRYAATLTNDVIISTGLAIAGRALALVFPASFLLKRLQYHDVNI |
| | LSYLFCLHQDLTKTTVSNCRVSSIYGLMVVICSMGLDSVLLLLSYVLILCTVLSTASK |
| | AERVRALNTCISHICAVLTFYTPMIGLSMTHRYGQNASSTVHVLMANVYLLVPPLMNP |
| | VVYSVKTKQIRDRIFNKFKKHEV |

Further analysis of the NOV71a protein yielded the following properties shown in Table 71B.

TABLE 71B
Protein Sequence Properties NOV71a

| | |
|---|---|
| PSort | 0.6000 probability located in plasma membrane; 0.4047 |
| analysis: | probability located in mitochondrial inner membrane; |
| | 0.4000 probability located in Golgi body; 0.3480 |
| | probability located in mitochondrial intermembrane space |
| SignalP | No Known Signal Sequence Predicted |
| analysis: | |

A search of the NOV71a protein against the Geneseq database, a proprietary database that contains sequences published in patents and patent publication, yielded several homologous proteins shown in Table 71C.

TABLE 71C
Geneseq Results for NOV71a

| | | | Identities/ | |
|---|---|---|---|---|
| | | NOV71a | Similarities for | |
| Geneseq | Protein/Organism/Length | Residues/ | the Matched | Expect |
| Identifier | [Patent #, Date] | Match Residues | Region | Value |
| AAG72605 | Human OR-like polypeptide | 1 . . . 309 | 295/310 (95%) | e−163 |
| | query sequence, SEQ ID NO: | 4 . . . 313 | 298/310 (95%) | |
| | 2286 - Homo sapiens, 318 aa. | | | |
| | [WO200127158-A2, 19 APR | | | |
| | 2001] | | | |
| AAG71519 | Human olfactory receptor | 1 . . . 309 | 295/310 (95%) | e−163 |
| | polypeptide, SEQ ID NO: 1200 - | 4 . . . 313 | 298/310 (95%) | |
| | Homo sapiens, 318 aa. | | | |
| | [WO200127158-A2, 19 APR | | | |
| | 2001] | | | |
| AAU24683 | Human olfactory receptor | 5 . . . 308 | 178/304 (58%) | e−102 |
| | aa. [WO200168805-A2, 20 SEP | 9 . . . 312 | 235/304 (76%) | |
| | 2001] | | | |
| AAG71715 | Human olfactory receptor | 5 . . . 308 | 178/304 (58%) | e−102 |
| | polypeptide, SEQ ID NO: 1396 - | 9 . . . 312 | 235/304 (76%) | |
| | Homo sapiens, 314 aa. | | | |
| | [WO200127158-A2, 19 APR | | | |
| | 2001] | | | |
| ABB44526 | Human GPCR4a polypeptide | 5 . . . 308 | 169/304 (55%) | 2e−96 |
| | SEQ ID NO 11 - Homo sapiens, | 6 . . . 309 | 227/304 (74%) | |
| | 315 aa. [WO200174904-A2, | | | |
| | 11 OCT 2001] | | | |

In a BLAST search of public sequence databases, the NOV71a protein was found to have homology to the proteins shown in the BLASTP data in Table 71D.

TABLE 71D
Public BLASTP Results for NOV71a

| | | NOV71a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H344 | Olfactory receptor 51I2 | 13 . . . 308 | 154/296 (52%) | 2e−91 |
| | (HOR5′beta12) - Homo sapiens | 12 . . . 307 | 221/296 (74%) | |
| | (Human), 312 aa. | | | |
| Q9H2C8 | ODORANT RECEPTOR | 2 . . . 308 | 160/307 (52%) | 5e−89 |
| | HOR3′BETA1 - Homo sapiens | 10 . . . 316 | 216/307 (70%) | |
| | Human), 321 aa. | | | |
| Q9H343 | Olfactory receptor 51I1 | 5 . . . 312 | 156/309 (50%) | 9e−89 |
| | (HOR5′beta11) - Homo sapiens | 5 . . . 313 | 223/309 (71%) | |
| | (Human), 314 aa. | | | |
| AAL35109 | PROSTATE-SPECIFIC G | 13 . . . 309 | 148/297 (49%) | 2e−86 |
| | PROTEIN-COUPLED | 11 . . . 307 | 207/297 (68%) | |
| | RECEPTOR RA1C - Mus | | | |
| | musculus (Mouse), 320 aa. | | | |
| Q924X8 | OLFACTORY RECEPTOR S85 - | 2 . . . 304 | 150/303 (49%) | 1e−85 |
| | Mus musculus (Mouse), 314 aa. | 3 . . . 305 | 221/303 (72%) | |

PFam analysis predicts that the NOV71a protein contains the domains shown in the Table 71E.

TABLE 71E
Domain Analysis of NOV71a

| | | Identities/ | |
|---|---|---|---|
| | | Similarities | |
| | NOV71a | for the | |
| Pfam Domain | Match Region | Matched Region | Expect Value |
| 7tm_1: | 42 . . . 138 | 24/99 (24%) | 7.8e−14 |
| domain 1 of 1 | | 67/99 (68%) | |

The NOV72 clone was analyzed, and the nucleotide and predicted polypeptide sequences are shown in Table 72A.

TABLE 72A
NOV72 Sequence Analysis

| | SEQ ID NO:209 | 1004 bp | |
|---|---|---|---|
| NOV72a, | CTTCTCATCTTTTCCCTCAAATACTGGGATGTCCATTCTCAATACCTCTGAAATGGAA | | |
| CG59211-01 DNA Sequence | | | |
| | ATCTCTATTTTCTACTTGGTTGGGATCCCAGGTTTGGAGCATGCCAATATTTGGATCT | | |
| | CTATCCCCATATGTCTCATGTACACTGTTGCTATCCTAGGGAATTGTACCATTCTGTT | | |
| | TTTCATAAAAACACAGCCTTCTTTGCATGAGCCCATGTACTATTTTCTCTCCATGTTG | | |
| | GCTCTCTCTGACCTGGGACTATCCCTCTCCTCTCTCCCTACCATGTTAACGATTTTCC | | |
| | TGTTCAATGCTCCAGGAATTTCCCCTGATGCCTGTATTGCTCAAGAGTTTTTCATCCA | | |
| | TGGATTCTCAGCTATGGAGTCATCTGTACTTCTTATAATGTCCTTTGATCGCTTTATT | | |
| | GCCATCTGCAACCCCCTGAGATACACTTCCATCCTCACCAGTCCCAGAGTCATTCAAA | | |
| | TTGGGCTTGCTTTTTCTCTCAAAAATCTTTTGTTCATCCTCCCATTTCCTTTCACTCT | | |
| | AAAACATCTAAAATATTGTAAGAAGAACCTCCTGTCCCAATCCTACTGCCTCCATCAA | | |
| | GATGTCATGAAACTGGCCTGCACTGACAACAAGGTCAACATCATCTATGGCTTATTTG | | |
| | TGGCTCTCACAGGCATCCTAGACTTGACATTTATTTTCATGTCCTACATCTTGATACT | | |
| | GAAAGCAGTGTTGAGCATAGCATCAAGAAAGAAAAGGCTCAAGGTCCTCAATACATGT | | |
| | GTTTCCCACATCTGTGCTGTGCTCATCTTCTATGTGCCCATTATCTCCCTAGCTGTCA | | |
| | TCTACCGGTTTGCCAAACACAGTTTCCCAATCACTAGGATCCTCATAGCTGATGCTTT | | |
| | TCTGCTGGTGCCTCCATTGATGAACCCCATTGTATACTGTGTGAAGAGCCAGCAGATA | | |
| | AGAAATCTTCTCTTAGAAAAACTGTGCCAGAAGCAAAGCTGAAGCGGATGCTTAACCA | | |
| | CATGATGCTTAACCCAAA | | |
| | ORF Start: ATG at 29 | | ORF Stop: TGA at 968 |
| | SEQ ID NO:210 | 313 aa | MW at 35313.1 kD |

| | |
|---|---|
| NOV72a, | MSILNTSEMEISIFYLVGIPGLEHANIWISIPICLMYTVAILGNCTILFFIKTEPSLH |
| CG59211-01 Protein Sequence | |
| | EPMYYFLSMLALSDLGLSLSSLPTMLRIFLFNAPGISPDACIAQEFFIHGFSAMESSV |
| | LLIMSFDRFIAICNPLRYTSILTSARVIQIGLAFSLKNVLLILPFPFTLKHLKYCKKN |
| | LLSQSYCLHQDVMKLACTDNKVNIIYGLFVALTGILDLTFTEMSYMLTLKAVLSIASR |
| | KKRLKVLNTCVSHICAVLTFYVPIISLAVIYRFAKHSFPTTRTLIADAFLLVPPLMNP |
| | IVYCVKSQQIRNLVLEKLCQKQS |

Further analysis of the NOV72a protein yielded the following properties shown in Table 72B.

TABLE 72B
Protein Sequence Properties NOV72a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.0300 probability |
| | located in mitochondrial inner membrane |
| SignalP | Likely cleavage site between residues 44 and 45 |
| analysis: | |

A search of the NOV72a protein against the Geneseq database, a proprietary database that contains sequences published in patents and patent publication, yielded several homologous proteins shown in Table 72C.

TABLE 72C
Geneseq Results for NOV72a

| | | | Identities/ | |
|---|---|---|---|---|
| | | NOV72a | Similarities for | |
| Geneseq | Protein/Organism/Length | Residues/ | the Matched | Expect |
| Identifier | [Patent #, Date] | Match Residues | Region | Value |
| AAG71564 | Human olfactory receptor | 1 . . . 313 | 312/313 (99%) | e−177 |
| | polypeptide, SEQ ID NO: 1245 - | 5 . . . 317 | 312/313 (99%) | |
| | Homo sapiens, 322 aa. | | | |
| | [WO200127158-A2, 19 APR 2001] | | | |
| AAU24573 | Human olfactory receptor | 1 . . . 312 | 225/312 (72%) | e−131 |
| | AOLFR63 - Homo sapiens, 313 aa. | 1 . . . 312 | 272/312 (87%) | |
| | [WO200168805-A2, 20 SEP 2001] | | | |
| AAG71721 | Human olfactory receptor | 1 . . . 311 | 236/312 (75%) | e−131 |
| | polypeptide, SEQ ID NO: 1402 - | 1 . . . 311 | 267/312 (84%) | |
| | Homo sapiens, 316 aa. | | | |
| | [WO200127158-A2, 19 APR 2001] | | | |
| AAU24682 | Human olfactory receptor | 1 . . . 308 | 224/308 (72%) | e−131 |
| | AOLFR181 - Homo sapiens, 312 | 1 . . . 306 | 265/308 (85%) | |
| | aa. [WO200168805-A2, 20 SEP | | | |
| | 2001] | | | |
| AAG71701 | Human olfactory receptor | 1 . . . 308 | 224/308 (72%) | e−131 |
| | polypeptide, SEQ ID NO: 1382 - | 1 . . . 306 | 265/308 (85%) | |
| | Homo sapiens, 312 aa. | | | |
| | [WO200127158-A2, 19 APR 2001] | | | |

In a BLAST search of public sequence databases, the NOV72a protein was found to have homology to the proteins shown in the BLASTP data in Table 72D.

TABLE 72D
Public BLASTP Results for NOV72a

| | | NOV72a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H344 | Olfactory receptor 51I2 | 12 . . . 304 | 152/294 (51%) | 6e−90 |
| | (HOR5′beta12) - Homo sapiens | 10 . . . 303 | 219/294 (73%) | |
| | (Human), 312 aa. | | | |
| Q9EQQ7 | MOR 3′BETA4 - Mus musculus | 1 . . . 309 | 159/310 (51%) | 9e−89 |
| | (Mouse), 319 aa. | 1 . . . 310 | 219/310 (70%) | |
| Q9H343 | Olfactory receptor 51I1 | 4 . . . 313 | 154/311 (49%) | 9e−89 |
| | (HOR5′beta11) - Homo sapiens | 4 . . . 314 | 226/311 (72%) | |
| | (Human), 314 aa. | | | |
| CAC38935 | SEQUENCE 9 FROM PATENT | 5 . . . 305 | 153/302 (50%) | 2e−87 |
| | WO0131014 - Homo sapiens | 6 . . . 306 | 217/302 (71%) | |
| | (Human), 318 aa. | | | |
| CAC37756 | SEQUENCE 1 FROM PATENT | 5 . . . 305 | 153/302 (50%) | 3e−87 |
| | WO0125434 - Homo sapiens | 5 . . . 305 | 217/302 (71%) | |
| | (Human), 317 aa. | | | |

PFam analysis predicts that the NOV72a protein contains the domains shown in the Table 72E.

TABLE 72E
Domain Analysis of NOV72a

| | | Identities/ | | |
|---|---|---|---|---|
| | | Similarities | | |
| | NOV72a | for the | | |
| Pfam Domain | Match Region | Matched Region | | Expect Value |
| DUF40: domain | 109 . . . 134 | 10/26 | (38%) | 0.38 |
| 1 of 1 | | 20/26 | (77%) | |
| 7tm_1: | 43 . . . 144 | 27/107 | (25%) | 1.6e−15 |
| domain 1 of 2 | | 71/107 | (66%) | |
| 7tm_1: | 212 . . . 293 | 16/93 | (17%) | 4.7 |
| domain 2 of 2 | | 56/93 | (60%) | |
| Sina: domain 1 of 1 | 300 . . . 311 | 7/12 | (58%) | 1 |
| | | 10/12 | (83%) | |

### Example 73

The NOV73 clone was analyzed, and the nucleotide and predicted polypeptide sequences are shown in Table 73A.

TABLE 73A
NOV73 Sequence Analysis

| | SEQ ID NO:211 | 1581 bp | |
|---|---|---|---|
| NOV73a, | CTGGTGGGTTGGCGGCTAACGGCCGGAGACAAGAGCGGCCGCCACCATCTCCTCCAAT | | |
| CG59276-01 DNA Sequence | | | |
| | GGAAGGGAGACAGGGGCGGGCTTAATGACGGAAGGAGCATGGCGTGGAGACACCTGAA | | |
| | AAAGCGGGCCCAGGATGCTCTCATCATCCTGGGGGCAGGAGGACTTCTCTTCGCCTCC | | |
| | TACCTGATGGCCACGGGAGATGAGCGTTTCTATGCTGAACACCTGATGCCGACTCTGC | | |
| | AGGGGCTGCTGGACCCGGAGTCAGCCCACAGACTGGCTGTTCGCTTCACCTCCCTGGG | | |
| | GCTCCTTCCACGGGCCAGATTTCAAGACTCTGACATGCTGGAAGTGAGAGTTCTGGGC | | |
| | CATAAATTCCGAAATCCAGTAGGAATTGCTGCAGCATTTGACAAGCATGGGGAAGCCG | | |
| | TGGACGGACTTTATAAGATGGGCTTTGGTTTTGTTGAGATAGGAAGTGTGACTCCAAA | | |
| | ACCTCAGGAAGGAAACCCTAGACCCAGAGTCTTCCGCCTCCCTGAGGACCAAGCTGTC | | |
| | ATTAACAGGTATGGATTTAACAGTCACGGGCTTTCAGTGGTGGAACACAGGTTACGGG | | |
| | CCAGACAGCAGAAGCAGGCCAAGCTCACAGAAGATGGACTGCCTCTGGGGGTCAACTT | | |
| | GGGGAAGAACAAGACCTCAGTGGACGCCGCGGAGGACTACGCAGAAGGGGTGCGCGTA | | |
| | CTGGGCCCCCTGGCCGACTACCTGCTGGTGAATGTGTCCAGCCCCAACACTGCCGGGC | | |
| | TGCGGAGCCTTCAGGGAAAGGCCGAGCTGCGCCGCCTGCTGACCAAGGTGCTCCAGGA | | |
| | GAGGGATGGCTTGCGGAGAGTGCACAGGCCGGCAGTCCTGGTGAAGATCGCTCCTGAC | | |
| | CTCACCAGCCAGGATAAGGAGGACATTGCCAGTGTGGTCAAAGAGTTGGGCATCGATG | | |
| | GGCTGATTGTTACGAACACCACCGTGAGTCGCCCTGCGGGCCTCCAGGGTGCCCTGCG | | |
| | CTCTGAAACAGGAGGGCTGAGTGGGAAGCCCCTCCGGGATTTATCAACTCAAACCATT | | |
| | CGGGAGATGTATGCACTCACCCAAGGCAAGGTTTCCCGTCGACTTCCCATAATTGGGG | | |
| | TTGGTGGTGTGAGCAGCGGGCAGGACGCGCTGGAGAAGATCCGGGCAGGGGCCTCCCT | | |
| | GGTGCAGCTGTACACGGCCCTCACCTTCTGGGGGCCACCCGTTGTGGGCAAAGTCAAG | | |
| | CGGGAACTGGAGGCCCTTCTGAAGGAGCAGGGCTTTGGCGGAGTCACAGATGCCATTG | | |
| | GAGCAGATCATCGGAGCATGAGGAAACGGGCAGAGAAGCGGCTGATTGTCCAGTCCCC | | |
| | CTGCGTGGAGGCTGCTTGGCTGGGCTCCAGCCCAGCGGTGGTGGGTCAGTTGGCACCT | | |
| | GGTGGTCTGCTGGTGGTCAGTTTGGGAATTTCCAGGTACGATTGTTTTCAGGCACTGT | | |
| | TCTTTGACTTGGTTGCAGAAAAACAGATTTTGCAACACTTTCCAAGGACACAGTGTTA | | |
| | CCACTCCCTCACCCTGCCATGGCCTCTTGGTTCTGCTTTTAACTTCTGAGCCTCAGGG | | |
| | AGTCCATCTTGTCTG | | |
| | ORF Start: ATG at 97 | | ORF Stop: TGA at 1555 |
| | SEQ ID NO:212 | 486 aa | MW at 52982.6 kD |

| | |
|---|---|
| NOV73a, | MAWRHLKKRAQDAVIILGGGGLLFASYLMATGDERFYAEHLMPTLQGLLDPESAHRLA |
| CG59276-01 Protein Sequence | |
| | VRFTSLGLLPRARFQDSDMLEVRVLGHKFRNPVGIAAGFDKHGEAVDGLYKMGFGFVE |
| | IGSVTPKPQEGNPRPRVFRLPEDQAVINRYGFNSHGLSVVEHRLRARQQKQAKLTEDG |
| | LPLGVNLGKNKTSVDAAEDYAEGVRVLGPLADYLVVNVSSPNTAGLRSLQGKAELRRL |
| | LTKVLQERDCLRRVHRPAVLVKIAPDLTSQDKEDTASVVKELGZDCLIVTNTTVSRPA |
| | GLQGALRSETGGLSGKPLRDLSTQTIREMYALTQGKVSRRVPIIGVGGVSSGQDALEK |
| | IRAGASLVQLYTALTFWGPPVVGKVKRELEALLKEQGFGGVTDAIGADHRRMRKRAEK |
| | RLIVQSPCVEAAWLGSSPAVVGQLGPGGLLVVSLGISRYDCFQALFFDLVAEKQILQH |
| | FPRTQCYHSLTLPWPLGSAFNF |

Further analysis of the NOV73a protein yielded the following properties shown in Table 73B.

TABLE 73B
Protein Sequence Properties NOV73a

| | |
|---|---|
| Psort | 0.8110 probability located in plasma membrane; 0.6400 |
| analysis: | probability located in endoplasmic reticulum (membrane); |
| | 0.3700 probability located in Golgi body; 0.1839 probability |
| | located in microbody (peroxisome) |
| SignalP | No Known Signal Sequence Predicted |
| analysis: | |

A search of the NOV73a protein against the Geneseq database, a proprietary database that contains sequences published in patents and patent publication, yielded several homologous proteins shown in Table 73C.

TABLE 73C
Geneseq Results for NOV73a

| | | | Identities/ | |
|---|---|---|---|---|
| | | NOV73a | Similarities for | |
| Geneseq | Protein/Organism/Length | Residues/ | the Matched | Expect |
| Identifier | [Patent #, Date] | Match Residues | Region | Value |
| AAB70780 | Tobacco dihydro-orotase protein - | 36 . . . 398 | 199/383 (51%) | e−101 |
| | Nicotiana tabacum, 458 aa. | 81 . . . 458 | 257/383 (66%) | |
| | [WO200118190-A2, 15 MAR 2001] | | | |
| AAG01301 | Human secreted protein, SEQ ID | 1 . . . 144 | 143/144 (99%) | 3e−79 |
| | NO: 5382 - Homo sapiens, 144 aa. | 1 . . . 144 | 144/144 (99%) | |
| | [EP1033401-A2, 6 SEP 2000] | | | |
| AAG91420 | C glutamicum protein fragment SEQ | 76 . . . 396 | 131/328 (39%) | 6e−60 |
| | ID NO: 5174 - Corynebacterium | 60 . . . 366 | 190/328 (56%) | |
| | glutamicum, 371 aa. [EP1108790- | | | |
| | A2, 20 JUN 2001] | | | |
| AAB46597 | C. glutamicum dihydroorotate | 76 . . . 396 | 131/328 (39%) | 6e−60 |
| | dehydrogenase protein - | 10 . . . 316 | 190/328 (56%) | |
| | Corynebacterium glutamicum, 321 | | | |
| | aa. [DE19929364-A1, 28 DEC | | | |
| | 2000] | | | |
| AAB80123 | Corynebacterium glutamicum MP | 76 . . . 396 | 131/328 (39%) | 1e−59 |
| | protein sequence SEQ ID NO:980 - | 23 . . . 329 | 190/328 (56%) | |
| | Corynebacterium glutamicum, 334 | | | |
| | aa. [WO200100843-A2, 4 JAN | | | |
| | 2001] | | | |

In a BLAST search of public sequence databases, the NOV73a protein was found to have homology to the proteins shown in the BLASTP data in Table 73D.

TABLE 73D
Public BLASTP Results for NOV73a

| | | NOV73a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q02127 | Dihydroorotate dehydrogenase, | 1 . . . 399 | 392/399 (98%) | 0.0 |
| | mitochondrial precursor (EC 1.3.3.1) | 2 . . . 396 | 394/399 (98%) | |
| | (Dihydroorotate oxidase) | | | |
| | (DHOdehase) - Homo sapiens | | | |
| | (Human), 396 aa (fragment). | | | |
| PC1219 | dihydroorotate oxidase (EC 1.3.3.1) | 1 . . . 399 | 388/399 (97%) | 0.0 |
| | precursor - human, 397 aa. | 3 . . . 397 | 393/399 (98%) | |
| Q63707 | Dihydroorotate dehydrogenase, | 1 . . . 399 | 350/399 (87%) | 0.0 |
| | mitochondrial precursor (EC 1.3.3.1) | 1 . . . 395 | 369/399 (91%) | |
| | (Dihydroorotate oxidase) | | | |
| | (DHOdehase) - Rattus norvegicus | | | |
| | (Rat), 395 aa. | | | |
| O35435 | Dihydroorotate dehydrogenase, | 1 . . . 399 | 346/399 (86%) | 0.0 |
| | mitochondrial precursor (EC 1.3.3.1) | 1 . . . 395 | 366/399 (91%) | |
| | (Dihydroorotate oxidase) | | | |
| | (DHOdehase) - Mus musculus | | | |
| | (Mouse), 395 aa. | | | |
| Q9FZM9 | DIHYDROOROTATE | 29 . . . 398 | 206/394 (52%) | e−101 |
| | DEHYDROGENASE - Oryza sativa | 79 . . . 468 | 261/394 (65%) | |
| | (Rice), 468 aa. | | | |

PFam analysis predicts that the NOV73a protein contains the domains shown in the Table 73E.

TABLE 73E
Domain Analysis of NOV73a

| | | Identities/ | |
|---|---|---|---|
| | | Similarities | |
| | NOV73a | for the | |
| Pfam Domain | Match Region | Matched Region | Expect Value |
| DHOdehase: | 77 . . . 381 | 183/331 (55%) | 1.9e−169 |
| domain 1 of 1 | | 282/331 (85%) | |

### Example 74

The NOV74 clone was analyzed, and the nucleotide and predicted polypeptide sequences are shown in Table 74A.

TABLE 74A
NOV74 Sequence Analysis

| | SEQ ID NO:213 | 1875 bp | |
|---|---|---|---|
| NOV74a, | ATGGCCGCAGCCTCGCCTCTGCGCGACTGCCAGGCCTGGAAGGATGCGAGGCTCCCGC | | |
| CG59268-01 DNA Sequence | | | |
| | TCTCCACCACAAGCAACGAAGCCTGCAAGCTGTTCGATGCCACGCTGACCCAGTATGT | | |
| | AAAATGGACCAATGACAAGACTCTCGCTGGCATCGAGGGCTGCCTGTCAAAGCTCAAA | | |
| | GCAGCAGATCCAACCTTTGTGATGGGCCACGCCATGGCTACTGGCCTTGTGCTGATTG | | |
| | GCACTGGAAGCTCCGTGAAGCTGGACAAAGAGCTGGACCTGGCTGTGAAGACAATGGT | | |
| | GGAGATTTCAAGAACCCAGCCGCTGACAAGGCGGGAGCAGCTGCACGTGTCTGCAGTA | | |
| | GAGACATTTGCCAATGGGAACTTTCCGAAAGCCTGTGAACTATGGGAACAGATTCTCC | | |
| | AGGACCACCCGACAGACATGTTGGCCCTGAAATTTTCCCATGATGCTTATTTTTACCT | | |
| | GGGCTATCAGGAACAGATGAGAGATTCTGTTGCTCGAATTTACCCCTTCTGCACACCT | | |
| | GACATCCCCCTAAGCAGCTATGTGAAAGGCATCTACTCTTTTGGCTTGATGGAAACCA | | |
| | ACTTCTACGACCAGGCAGAAAAACTCGCCAAAGAGGCACCAACTCTTTGTCTTCAACA | | |
| | CCAGCACCCCACAGACAACTACTGGGCAGGAAAAGCAGGCTGTGATGGGGCCAGGAGT | | |
| | GGTAACACATGGGCTCTGTGTCTGCAGCCCCAGGCTGACGCATGGTCCCTGCACACCG | | |
| | TCGCTCACATCCACGAGATGAAAGCAGAGATCAAGGATGGGTTGGAATTCATGCAGCA | | |
| | CTCAGAGACCTTCTGGAAGGACTCTGATATGTTGGCTTGTCATAACTATTGGCACTGG | | |
| | GCTTTATATCTGATTGAGAAGGGTTTAATAACGACAACTTTATTCTTCCAGGGCGAAT | | |
| | ATGAGGCCGCGCTGACCATCTACGATACCCACATCCTTCCCAGCCTGCAGGCCAACGA | | |
| | TGCAATGCTGGACGTGGTGGACAGCTGCTCCATGCTCTACCGCCTGCAGATGGAAGGA | | |
| | GTGTCTGTGGGCCAGCGGTGGCAGGATGTCCTGCCTGTGGCCCGGAAGCACAGCCGAG | | |
| | ACCACATCCTGCTGTTCAATGACGCACACTTCCTGATGGCATCCCTGGGTGCACACGA | | |
| | CCCCCAGACCACACAGGAGCTGCTGACCACCCTGCGGGACGCCAGCGAGTATGCAGAG | | |
| | GGGCCTTCTCGGGGTGGGGGTCCTCACCCTGCCGAGAGGTGCCAGGCCTTTGCCTGTA | | |
| | TTATCAGCAATCCTGACGGTTCTGTTAGATTGGCACTGTTATGCCTGCTTACAGATGA | | |
| | GCAAACTGAGGCTGGAAGATCCCCAGGGGAGAACTGCCAGCACCTCCTGGCCCGAGAC | | |
| | GTGGGGCTGCCCCTGTGCCAGGCCCTGGTGGAGGCTGAGGACGGGAACCCTGACCGCG | | |
| | TCCTGGAGCTGCTCCTGCCCATCCGCTACCGGATCGTCCAGCTCGGTGGGAGCAATGC | | |
| | CCAGAGAGACGTCTTCAACCAGCTGCTGATTCACGCGGCCTTAAACTGCACCTCCAGC | | |
| | GTCCATAAGAACGTAGCCCGGAGCCTTCTGATGGAGCGTGATGCCTTGAAGCCCAACT | | |
| | CGCCCCTCACCGAGCGGCTCATCCGCAAGGCAGCTACCGTCCACCTCATGCAGAAGCC | | |
| | TTCTACCCGCCAACCCCCACTGCAGGCTGCTCTCTCCATGGAAGGAGGCGGCGGCCGC | | |
| | GATGAGCCTTCAGCCTGCCGGGCAGGGGACGTGAACATGGATGACCCTAACAAGGAAG | | |
| | TGATTTAATGTTTCCCTGA | | |
| | ORF Start: ATG at 1 | | ORF Stop: TGA at 1873 |
| | SEQ ID NO:214 | 624 aa | MW at 69393.3 kD |

| | |
|---|---|
| NOV74a, | MAAASPLRDCQAWKDARLPLSTTSNEACKLFDATLTQYVKWTNDKSLGGTEGCLSKLK |
| CG59268-01 Protein Sequence | |
| | AADPTFVMGHAMATGLVLIGTGSSVKLDKELDLAVKTMVEISRTQPLTRREQLHVSAV |
| | ETFANGNFPKACELWEQILQDHPTDMLALKFSHDAYFYLGYQEQMRDSVARIYPFWTP |
| | DIPLSSYVKGTYSEGLMETNFYDQAEKLAKEAPTLCLQHQHPTDNYWAGKAGCDGARS |
| | GNTWALCLQPQADAWSVHTVAHIHEMKAEIKDGLEFMQHSETFWKDSDMLACHNYWIW |
| | ALYLIEKGLIRRTLFFQGEYEAALTIYDTHILPSLQANDANLDVVDSCSMLYRLQMEG |
| | VSVGQRWQDVLPVARKHSRDHILLFNDAHFLMASLGAHDPQTTQELLTTLRDASEYAE |
| | GPSRGGGPHPAERCQAFACIISNPDGSVRLALLCLLTDEQTEAGRSPGENCQHLLARD |
| | VGLPLCQALVEAEDGNPDRVLELLLPIRYRIVQLGGSNAQRDVFNQLLIHAALWCTSS |
| | VHKNVARSLLMERDALKPNSPLTERLTRKAATVHLMQKPSTRQPPLQAALSMEGGGGR |
| | DEPSACRAGDVNMDDPKKEGKSLLLRRCCCSGCSVEMEGDLMFP |

Further analysis of the NOV74a protein yielded the following properties shown in Table 74B.

TABLE 74B
Protein Sequence Properties NOV74a

| | |
|---|---|
| PSort | 0.4328 probability located in mitochondrial matrix space; |
| analysis: | 0.3000 probability located in microbody (peroxisome); |
| | 0.1137 probability located in mitochondrial inner membrane; |
| | 0.1137 probability located in mitochondrial intermembrane |
| | space |
| SignalP | No Known Signal Sequence Predicted |
| analysis: | |

A search of the NOV74a protein against the Geneseq database, a proprietary database that contains sequences published in patents and patent publication, yielded several homologous proteins shown in Table 74C.

TABLE 74C
Geneseq Results for NOV74a

| | | | | |
|---|---|---|---|---|
| | | | Identities/ | |
| | | NOV74a | Similarities for | |
| Geneseq | Protein/Organism/Length | Residues/ | the Matched | Expect |
| Identifier | [Patent #, Date] | Match Residues | Region | Value |

| | | | | | |
|---|---|---|---|---|---|
| AAM41338 | Human polypeptide SEQ ID NO | 1 . . . 559 | 463/559 | (82%) | 0.0 |
| | 6269 - Homo sapiens, 478 aa. | 10 . . . 478 | 466/559 | (82%) | |
| | [WO200153312-A1, 26 JUL | | | | |
| | 2001] | | | | |
| AAM39552 | Human polypeptide SEQ ID NO | 1 . . . 529 | 434/529 | (82%) | 0.0 |
| | 2697 - Homo sapiens, 453 aa. | 1 . . . 439 | 437/529 | (82%) | |
| | [WO200153312-A1, 26 JUL | | | | |
| | 2001] | | | | |
| AAG02871 | Human secreted protein, SEQ ID | 1 . . . 102 | 102/102 | (100%) | 1e−52 |
| | NO: 6952 - Homo sapiens, 104 aa. | 1 . . . 102 | 102/102 | (100%) | |
| | [EP1033401-A2, 6 SEP 2000] | | | | |
| AAM40893 | Human polypeptide SEQ ID NO | 568 . . . 604 | 32/37 | (86%) | 2e−10 |
| | 5824 - Homo sapiens, 746 aa. | 1 . . . 37 | 32/37 | (86%) | |
| | [WO200153312-A1, 26 JUL | | | | |
| | 2001] | | | | |
| AAM40892 | Human polypeptide SEQ ID NO | 568 . . . 604 | 32/37 | (86%) | 2e−10 |
| | 5823 - Homo sapiens, 746 aa. | 1 . . . 37 | 32/37 | (86%) | |
| | [WO200153312-A1, 26 JUL | | | | |
| | 2001] | | | | |

In a BLAST search of public sequence databases, the NOV74a protein was found to have homology to the proteins shown in the BLASTP data in Table 74D.

TABLE 74D
Public BLASTP Results for NOV74a

| | | NOV74a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH18918 | HYPOTHETICAL 45.7 KDA | 66 . . . 559 | 399/494 (80%) | 0.0 |
| | PROTEIN - Homo sapiens | 1 . . . 404 | 402/494 (80%) | |
| | (Human), 404 aa. | | | |
| Q9NWP8 | KAIA2372 PROTEIN - Homo | 1 . . . 352 | 305/352 (86%) | e−172 |
| | sapiens (Human), 336 aa. | 1 . . . 310 | 308/352 (86%) | 3e−61 |
| Q9XW02 | Y54G11A.4 PROTEIN - | 4 . . . 556 | 165/557 (29%) | |
| | Caenorhabditis elegans, 497 aa. | 6 . . . 458 | 256/557 (45%) | |
| Q9XW01 | Y54G11A.7 PROTEIN - | 4 . . . 347 | 122/347 (35%) | 7e−53 |
| | Caenorhabditis elegans, 407 aa. | 6 . . . 305 | 177/347 (50%) | |
| Q98CS1 | MLR5032 PROTEIN - | 60 . . . 553 | 145/496 (29%) | 1e−43 |
| | Rhizobium loti (Mesorhizobium | 46 . . . 435 | 215/496 (43%) | |
| | loti), 440 aa. | | | |

PFam analysis predicts that the NOV74a protein contains the domains shown in the Table 74E.

TABLE 74E
Domain Analysis of NOV74a

| | | Identities/ | |
|---|---|---|---|
| Pfam | NOV74a | Similarities for | Expect |
| Domain | Match Region | the Matched Region | Value |
| Monooxygenase: domain | 225 . . . 410 | 28/238 (12%) | 6.4 |
| 1 of 1 | | 121/238 (51%) | |

### Example 75

The NOV75 clone was analyzed, and the nucleotide and predicted polypeptide sequences are shown in Table 75A.

TABLE 75A
NOV75 Sequence Analysis

| | SEQ ID NO:215 | 1851 bp | |
|---|---|---|---|
| NOV75a, | CAGCTACAGCAAACATCGTTCGAGATGTCCCACCAAGAGGGCAGCACAGGTGGCTTAC | | |
| CG59549-01 DNA Sequence | | | |
| | CAGACTTAGTGACTGAAAGCCTGTTCAGCAGCCCAGAGGAGCAGTCTGGAGTAGCAGC | | |
| | GGTGACGGCGCCCTCCTCACACATTCAAATGGCACCCACAGAGCCATCGACCGGAGAT | | |
| | GGTGGTGATACCAGGGATGGTGGTTTCCTGAACGATGCCAGCACAGAAAATCAAAACA | | |
| | CAGACTCAGAAAGTTCAAGTGAAGACGTCGAACTTGAAAGCATGGGTGAACGTTTATT | | |
| | TGGTTACCCGTTAGTGGGAGAGGAGACAGAAAGGGAGGAGGAAGAAGAAGAGATGGAG | | |
| | GAGGAAGGGGAGGAGGAACAACAGCCTCCGATGTGTCCACGATGCGCTGGCACCAACC | | |
| | ATGATCAGTGTTTGTTAGACGAGGATCAGGCGTTGGAGGAGTGGATTTCCTCAGAGAC | | |
| | ATCTGCCCTGCCCCGATCTCGCTGGCAAGTCCTTACTGCTCTTCGCCAGCGGCAGCTG | | |
| | GGTTCAAGTGCCCGCTTTGTATATGAGGCCTGTGGGGCAAGAACCTTTGTGCAGCGTT | | |
| | TCCGCCTGCAQTATCTTCTTGGAAGCCATGCCGGTTCTGTCAGTACCATACACTTTAA | | |
| | CCAGCGTGGCACCCGACTGGCCAGTAGCGGTGATGACTTAAGGGTGATAGTGTGGGAC | | |
| | TGGGTGCGGCAGAAQCCAGTACTGAACTTTGAGAGTGGTCACGATATTAATGTCATCC | | |
| | AGGCTAAGTTCTTTCCTAACTGTGGTGATTCCACTCTGGCCATGTGTGGCCATGATGG | | |
| | ACAGGTACGGGTAGCAGAACTAATTAATGCATCATATTGCGAGAATACTAAGCGTGTG | | |
| | GCCAAGCACAGGGGACCTGCCCACGAGTTGGCTCTGGAGCCAGACTCTCCTTATAAGT | | |
| | TCCTCACTTCAGGTGAAGATGCCGTTGTGTTCACCATTGACCTCAGGCAAGACCCGCC | | |
| | AGCTTCAAAAGTTGTGCTAACAAGAGAAAATGATAAGAAAGTCGCACTGTATACAATC | | |
| | TCTATGAATCCTGCCAATATTTACCAATTTGCAGTGGGTGGACATGATCAGTTTGTAA | | |
| | GGATTTATGACCAGAGGAGAATTGATAAGAAAGAAAACAATGGAGTACTCAAGAAATT | | |
| | CACTCCTCATCATCTCGTTTATTGTGATTTCCCAACAAACATCACCTCCGTTGTGTAC | | |
| | AGCCACGATCGCACAGAGCTCCTGGCCAGCTACAATGATGAAGATATTTACCTCTTCA | | |
| | ACTCCTCTCTCAGTGATGGTGCTCAATATGTTAAGAGATATAAGGGGCACAGAAATAA | | |
| | TGACACAATCAAATGTGTTAATTTCTATGGCCCCCGGAGTGAGTTTGTCGTGAGCGGT | | |
| | AGTGATTGTGGGCACGTCTTCTTCTGGGAGAAATCATCCTCCCAGATCATCCAGTTCA | | |
| | TGGAGGGGGACAGAGGATATATAGTAAACTGTCTTGAACCCCACCCTTACCTACCTGT | | |
| | GTTGGCGACCAGTGGCCTAGATCAGCATGTCAGCATCTGGACACCCACAGCTAAAACT | | |
| | GCCACTGAGCTTACTGGGTTAAAAGATGTGATTAAGAAGAACAAGCAGGAGCGAGATG | | |
| | AAGACAACTTGAACTATACGGACTCGTTTGACAACCGCATGCTTCGGTTCTTCGTGCC | | |
| | TCACCTGTTACAGAGAGCTCATCAACCCGGCTGGAGAGATCATGGAGCTGAGTTCCCA | | |
| | GATGAAGAAGAGTTGGATGAGTCTTCCAGCACCTCAGATACATCCGAGGAGGAGGGCC | | |
| | AAGATCGAGTGCAGTGCATACCATCCTGAAGGCCTCATATCCAGTCCAGCTAG | | |
| | ORF Start: ATG at 25 | | ORF Stop: TGA at 1825 |
| | SEQ ID NO:216 | 600 aa | MW at 67372.4 kD |

| | |
|---|---|
| NOV75a, | MSHQEGSTGGLPDLVTESLFSSPEEQSGVAAVTAASSDIEMAATEPSTGDGGDTRDGG |
| CG59549-01 Protein Sequence | |
| | FLNDASTENQNTDSESSSEDVELESMGEGLFGYPLVGEETEREEEEEEMEEEGEEEEQ |
| | PRMCPRCGGTNHDQCLLDEDQALEEWISSETSALPRSRWQVLTALRQRQLGSSARFVY |
| | EACGARTFVQRFRLQYLLGSHAGSVSTTHFNQRGTRLASSGDDLRVTVWDWVRQKPVL |
| | NFESGHDINVIQAKFFPNCGDSTLAMCGHDGQVRVAELINASYCENTKRVAKHRGPAH |
| | ELALEPDSPYKFLTSGEDAVVFTIDLRQDRPASKVVVTRENDKKVGLYTISMNPANIY |
| | QFAVGGHDQFVRIYDQRRIDKKENNGVLKKFTPHHLVYCDFPTNITCVVYSHDGTELL |
| | ASYNDEDTYLFNSSLSDGAQYVKRYKCHRNNDTIKCVNFYGFRSEFVVSGSDCGEVFF |
| | WEKSSSQIIQFMECDRGDTVNCLEPHPYLPVLATSGLDQHVRIWTPTAKTATELTCLK |
| | DVIKKNKQERDEDNLNYTDSFDNRNLRFFVRHLLQRAHQPGWRDHGAEFPDEEELDES |
| | SSTSDTSEEEGQDRVQCIPS |

Further analysis of the NOV75a protein yielded the following properties shown in Table 75B.

TABLE 75B
Protein Sequence Properties NOV75a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0442 probability located in |
| | microbody (peroxisome) |
| SignalP | No Known Signal Sequence Predicted |
| analysis: | |

A search of the NOV75a protein against the Geneseq database, a proprietary database that contains sequences published in patents and patent publication, yielded several homologous proteins shown in Table 75C.

TABLE 75C
Geneseq Results for NOV75a

| | | NOV75a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR85870 | WD-40 domain-contg. Mus | 95 . . . 589 | 295/495 (59%) | e−179 |
| | musculus protein-Mus musculus, | 333 . . . 815 | 372/495 (74%) | |
| | 816 aa. [WO9521252-A2, | | | |
| | 10 AUG. 1995] | | | |
| AAM73935 | Human bone marrow expressed | 1 . . . 157 | 157/157 (100%) | 2e−87 |
| | probe encoded protein SEQ ID NO: | 8 . . . 164 | 157/157 (100%) | |
| | 34241-Homo sapiens, 164 aa. | | | |
| | [WO200157276-A2, 9 AUG. 2001] | | | |
| AAM61216 | Human brain expressed single exon | 1 . . . 157 | 157/157 (100%) | 2e−87 |
| | probe encoded protein SEQ ID NO: | 8 . . . 164 | 157/157 (100%) | |
| | 33321-Homo sapiens, 164 aa. | | | |
| | [WO200157275-A2, 9 AUG. 2001] | | | |
| AAM34114 | Peptide #8151 encoded by probe | 1 . . . 157 | 157/157 (100%) | 2e−87 |
| | for measuring placental gene | 8 . . . 164 | 157/157 (100%) | |
| | expression-Homo sapiens, 164 aa. | | | |
| | [WO200157272-A2, 9 AUG. 2001] | | | |
| AAB57007 | Human prostate cancer antigen | 408 . . . 600 | 144/194 (74%) | 2e−80 |
| | protein sequence SEQ ID NO: 1585- | 22 . . . 214 | 162/194 (83%) | |
| | Homo sapiens, 214 aa. | | | |
| | [WO200055174-A1, 21 SEP. 2000] | | | |

In a BLAST search of public sequence databases, the NOV75a protein was found to have homology to the proteins shown in the BLASTP data in Table 75D.

TABLE 75D
Public BLASTP Results for NOV75a

| | | NOV75a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q12839 | H326 PROTEIN-Homo sapiens | 1 . . . 600 | 408/604 (67%) | 0.0 |
| | (Human), 597 aa. | 1 . . . 597 | 471/604 (77%) | |
| Q01078 | PROTEIN PC326-Mus musculus | 95 . . . 589 | 295/495 (59%) | e−178 |
| | (Mouse), 747 aa. | 264 . . . 746 | 372/495 (74%) | |
| Q9W091 | CG8001 PROTEIN-Drosophila | 68 . . . 587 | 178/533 (33%) | 1e−77 |
| | melanogaster (Fruit fly), 748 aa. | 209 . . . 711 | 280/533 (52%) | |
| Q96E00 | UNKNOWN (PROTEIN FOR | 1 . . . 246 | 141/249 (56%) | 8e−66 |
| | MGC: 9478)-Homo sapiens | 1 . . . 243 | 173/249 (68%) | |
| | (Human), 273 aa. | | | |
| Q9M1E5 | HYPOTHETICAL 54.0 KDA | 183 . . . 536 | 136/382 (35%) | 2e−62 |
| | PROTEIN-Arabidopsis thaliana | 42 . . . 419 | 209/382 (54%) | |
| | (Mouse-ear cress), 481 aa. | | | |

PFam analysis predicts that the NOV75a protein contains the domains shown in the Table 75E.

TABLE 75E
Domain Analysis of NOV75a

| | | Identities/ | |
|---|---|---|---|
| Pfam | NOV75a | Similarities for | Expect |
| Domain | Match Region | the Matched Region | Value |
| WD40: domain 1 of 7 | 188 . . . 224 | 13/37 (35%) | 0.0016 |
| | | 29/37 (78%) | |
| WD40: domain 2 of 7 | 231 . . . 269 | 12/39 (31%) | 11 |
| | | 26/39 (67%) | |
| WD40: domain 3 of 7 | 278 . . . 315 | 9/38 (24%) | 2.2e+02 |
| | | 24/38 (63%) | |
| WD40: domain 4 of 7 | 326 . . . 363 | 8/38 (21%) | 8.8 |
| | | 27/38 (71%) | |
| WD40: domain 5 of 7 | 382 . . . 418 | 5/37 (14%) | 12 |
| | | 27/37 (73%) | |
| WD40: domain 6 of 7 | 429 . . . 466 | 6/38 (16%) | 18 |
| | | 26/38 (68%) | |
| WD40: domain 7 of 7 | 473 . . . 509 | 11/37 (30%) | 0.51 |
| | | 22/37 (59%) | |

### Example 76

The NOV76 clone was analyzed, and the nucleotide and predicted polypeptide sequences are shown in Table 76A.

TABLE 76A
NOV76 Sequence Analysis

| | SEQ ID NO:217 | 7497 bp | |
|---|---|---|---|
| NOV76a, | ATGGTCTTGCTTCTTTGTCTATCTTGTCTGATTTTCTCCTGTCTGACCTTTTCCTGGT | | |
| CG59641-01 DNA Sequence | | | |
| | TAAAAATCTGGGGGAAAATGACGGACTCCAAGCCGATCACCAAGAGTAAATCAGAAGC | | |
| | AAACCTCATCCCGAGCCAGGAGCCCTTTCCAGCCTCTGATAACTCAGGGGAGACACCG | | |
| | CAGAGAAATGGGGAGGGCCACACTCTGCCCAACACACCCAGCCAGGCCGAGCCAGCCT | | |
| | CCCACAAAGGCCCCAAAGATGCCGGTCGGCGGAGAAACTCCCTACCACCCTCCCACCA | | |
| | GAAGCCCCCAAGAAACCCCCTTTCTTCCAGTGACGCAGCACCCTCCCCAGAGCTTCAA | | |
| | GCCAACGCGACTGGGACACAAGGTCTGGAGGCCACAGATACCAATGGCCTGTCCTCCT | | |
| | CAGCCAGGCCCCAGGGCCAGCAAGCTGGCTCCCCCTCCAAAGAAGACAAGAAGCAGGC | | |
| | AAACATCAAGAGGCAGCTGATGACCAACTTCATCCTGGGCTCTTTTGATGACTACTCC | | |
| | TCCGACGAGGACTCTGTTGCTGGCTCATCTCGTGAGTCTACCCCGAAGGGCAGCCGGC | | |
| | CCAGCTTGGGGGCCCTGTCCCTGGAGGCTTATCTGACCACAAGGCCGAGCATGTCGGG | | |
| | ACTCCACCTGGTGAAGAGGGGACGGGAACACAAGAAGCTGGACCTGCACAGAGACTTT | | |
| | ACCGTGGCTTCTCCCGCTGAGTTTGTCACACGCTTTGGGGGGGATCGGGTCATCGAGA | | |
| | AGGTGCTTATTGCCAACAACGGCATTGCCGCCGTGAAGTGCATGCGCTCCATCCGCAG | | |
| | GTGGGCCTATGAGATGTTCCGCAACGAGCGGGCCATCCGGTTTGTTGTGATGGTGACC | | |
| | CCCGAGGACCTTAAGGCCAACGCAGAGTACATCAAGATGGCGGATCATTACGTCCCCG | | |
| | TCCCAGGAGGGCCCAATAACAACAACTATGCCAACGTGGAGCTGATTGTGGACATTGC | | |
| | CAAGAGAATCCCCGTGCAGCCGGTGTGGGCTGGCTGGGGCCATGCTTCAGAAAACCCT | | |
| | AAACTTCCGGAGCTGCTGTGCAAGAATGGAGTTGCTTTCTTAGGCCCTCCCAGTGAGG | | |
| | CCATGTGGGCCTTAGGAGATAAGATCGCCTCCACCGTTGTCGCCCAGACGCTACAGGT | | |
| | CCCAACCCTGCCCTGGAGTGGAAGCGGCCTGACAGTGGAGTGGACAGAAGATGATCTG | | |
| | CAGCAGGGAAAAAGAATCAGTGTCCCAGAAGATGTTTATGACAAGGGTTGCGTGAAAG | | |
| | ACGTAGATGAGGGCTTGGAGGCAGCAGAAACAATTGGTTTTCCATTGATCATCAAAGC | | |
| | TTCTGAAGGTGGCGGAGGGAAGGGAATCCGGAAGGCTGAGAGTGCGGAGGACTTCCCG | | |
| | ATCCTTTTCAGACAAGTACAGAGTGAGATCCCAGGCTCGCCCATCTTTCTCATGAAGC | | |
| | TGGCCCAGCACGCCCGTCACCTGGAAGTTCAGATCCTCGCTGACCAGTATGGGAATGC | | |
| | TGTGTCTCTGTTTGGTCGCGACTGCTCCATCCAGCGGCGGCATCAGAAGATCGTTGAG | | |
| | GAAGCACCGGCCACCATCGCCCCGCTGGCCATATTCGAGTTCATGGAGCAGTGTGCCA | | |
| | TCCGCCTGGCCAAGACCGTGGGCTATGTGAGTGCAGGGACAGTGGAATACCTCTATAG | | |
| | TCAGGATGGCAGCTTCCACTTCTTGGAGCTGAATCCTCGCTTGCAGGTGGAACATCCC | | |
| | TGCACAGAAATGATTGCTGATGTTAATCTGCCGGCCGCCCAGCTACAGATCGCCATGG | | |
| | GCGTGCCACTGCACCGGCTGAAGGATATCCGGCTTCTGTATGGAGAGTCACCATGGGG | | |
| | AGTGACTCCCATTTCTTTTGAAACCCCCTCAAACCCTCCCCTCGCCCGAGGCCACGTC | | |
| | ATTGCCGCCAGAATCACCAGCGAAAACCCAGACGAGGGTTTTAAGCCGAGCTCCGGGA | | |
| | CTGTCCAGGAACTGAATTTCCGGAGCAGCAAGAACGTGTGGGGTTACTTCAGCGTGGC | | |
| | CGCTACTGGAGCCCTGCACCAGTTTGCGGATTCCCAATTTGGGCACTGCTTCTCCTGG | | |
| | GGAGAGAACCGGGAAGAGGCCATTTCGAACATGGTGGTGGCTTTGAAGGAACTGTCCA | | |
| | TCCGAGGCGACTTTAGGACTACCGTGGAATACCTCATTAACCTCCTGGACACCGAGAG | | |
| | CTTCCAGAACAACGACATCGACACCGGGTGGTTGGACTACCTCATTGCTGAGAAAGTG | | |
| | CAGGCGGAGAAACCGGATATCATGCTTGGGGTGGTATGCGGGGCCTTGAACGTGGCCG | | |
| | ATGCGATGTTCAGAACGTGCATGACAGATTTCTTACACTCCCTCCAAAGGCGCCAGGT | | |
| | CCTCCCAGCGGATTCACTACTGAACCTCGTAGATGTGGAATTAATTTACGGAGGTGTT | | |
| | AAGTACATTCTCAAGGTGGCCCGGCAGTCTCTGACCATGTTCGTTCTCATCATGAATG | | |
| | GCTCCCACATCGAGATTGATGCCCACCGCCTGAATGATCCGGGGCTCCTCCTCTCCTA | | |
| | CAATGGGAACAGCTACACCACCTACATGAAGGAAGAGGTTGACACTTACCGAATTACC | | |
| | ATCGGCAATAAGACGTGTGTGTTTGAGAAGGAGAACGATCCTACAGTCCTGAGATCCC | | |
| | CCTCGGCTGGGAAGCTGACACAGTACACAGTGGAGGATGGGGGCCACGTTGAGGCTGG | | |
| | GAGCAGCTACGCTGAGATGGAGGTGATGAAGATGATCATGACCCTGAACGTTCAGGAA | | |
| | AGAGGCCGGGTGAAGTACATCAAGCGTCCAGGTGCCGTGCTGGAAGCAGGCTGCGTGG | | |
| | TGGCCAGGCTGGAGCTCGATGACCCTTCTAAAGTCCACCCGGCTCAACCGTTCACAGG | | |
| | AGAACTCCCTGCCCAGCAGACACTGCCCATCCTCGGAGAGAAACTGCACCACGTCTTC | | |
| | CACAGCGTCCTGGAAAACCTCACCAACCTCATGAGTCGCTTTTCTCTGCCAGACCCCG | | |
| | TTTTTAGCATAAAGCTGAAGGAGTGGGTGCAGAAGCTCATGATGACCCTCCGGCACCC | | |
| | GTCACTGCCGCTGCTGGAGCTCCAGGAGATCATGACCAGCGTGGCAGGCCGCATCCCC | | |
| | GCCCCTGTGGAGAAGTCTGTCCGCAGGGTGATGGCCCAGTATGCCAGCAACATCACCT | | |
| | CGGTGCTGTGCCAGTTCCCCACCCAGCAGATAGCCACCATCCTGGACTGCCATCCAGC | | |
| | CACCCTGCAGCGGAAGGCTGATCGAGAGGTCTTCTTCATCAACACCCACAGCATCGTG | | |
| | CAGTTGGTCCAGAGATACCGCAGCGCGATCCGCGCCTATATGAAAACAGTGGTGTTGG | | |
| | ATCTCCTGAGAAGATACTTGCGTGTTGAGAGCAACGCAAGACATGCTGATGCCAACAC | | |
| | CAGTGGGATGGTGGGGGGCGTGAGGAGCCTGAGCTTTACCTCTGTGTGGTGTTTTGTC | | |
| | TCCCCCGAATCCCACTACGACAAGTGTGTGATAAACCTCAGGGAGCAGTTCAAGCCAG | | |
| | ACATGTCCCAGGTGCTGGACTGCATCTTCTCCCACGCACAGGTGGCCAAGAAGAACCA | | |
| | GCTGGTGATCATGTTGATCGATGAGCTGTGTGGCCCAGACCCTTCCCTGTCGGACGAG | | |
| | CTGATCTCCATCCTCAACGAGCTCACTCAGCTGAGCAAAAGCGAGCACTGCAAAGTGG | | |
| | CCCTCAGAGCCCGGCAGATCCTGATTGCCTCCCACCTCCCCTCCTACGAGCTGCGGCA | | |
| | TAACCACGTGGAGTCCATTTTCCTGTCTGCCATTGACATCTACGCCCACCAGTTCTGC | | |
| | CCCCACAACCTCAAGAAATTAATACTTTCGGAAACAACCATCTTCGACGTCCTGCCTA | | |
| | CTTTCTTCTATCACGCAAACAAAGTCGTGTGCATGGCGTCCTTGGAGCTTTACGTGCC | | |
| | GAGGGGCTACATCGCCTATGAGTTAAACAGCCTGCAGCACCGGCAGCTCCCGGACGGC | | |
| | ACCTGCGTGGTAGAATTCCAGTTCATCCTGCCGTCCTCCCACCCAAACCGGATGACCG | | |
| | TGCCCATCACCATCACCAACCCTGACCTGCTGAGGCACAGCACAGAGCTCTTCATGCA | | |
| | CAGCGGCTTCTCCCCACTGTGCCAGCGCATGGGAGCCATGGTAGCCTTCAGGAGATTC | | |
| | GAGCACTTCACCAGAAATTTTGATGAAGTCATCTCTTGCTTCGCCAACGTCCCCAAAG | | |
| | ACACCCCCCTCTTCACCGAGGCCCGCACCTCCCTATACTCCGAGGATGACTGCAAGAG | | |
| | CCTCAGAGAAGAGCCCATCCACATTCTGAATGTGTCCATCCAGTGTGCAGACCACCTG | | |
| | GAGGATGAGGCACTGGTGCCGATTTTACGGACATTCGTACAGTCCAAGAAAAATATCC | | |
| | TTGTGGATTATCCACTCCGACGAATCACATTCTTGATTGCCCAAGACTTTGCAGAAGA | | |
| | TCGCATTTACCGTCACTTCGAACCTGCCCTGGCCTTCCAGCTGGAACTTAACCGGATG | | |
| | CGTAACTTCGATCTGACCGCCGTGCCCTGTGCCAACCACAAGATGCACCTTTACCTGG | | |
| | GTGCTGCCAAGGTGAAGGAAGGTGTGGAAGTGACGGACCATAGGTTCTTCATCCGCGC | | |
| | CATCATCAGGCACTCTGACCTGATCACAAAGGAAGCCTCCTTCGAATACCTGCAGAAC | | |
| | GAGGGTGAGCGGCTGCTCCTGGAGGCCATGGACGAGCTGGAGGTGGCGTTCAATAACA | | |
| | CCAGCGTGCGCACCGACTGCAACCACATCTTCCTCAACTTCGTGCCCACTGTCATCAT | | |
| | GGACCCCTTCAAGATCGAGGAGTCCGTGCGCTACATGGTTATGCGCTACGGCAGCCGG | | |
| | CTGTGGAAACTCCGTGTGCTACAGGCTGAGGTCAAGATCAACATCCGCCAGACCACCA | | |
| | CCGGCAGTGCCGTTCCCATCCGCCTGTTCATCACCAATGAGTCGGGCTACTACCTGGA | | |
| | CATCAGCCTCTACAAAGAAGTGACTGACTCCACATCTGGAAATATCATCTTTCACTCC | | |
| | TTCGGCAACAAGCAAGGGCCCCAGCACGGGATGCTGATCAATACTCCCTACGTCACCA | | |
| | AGGATCTGCTCCAGGCCAAGCGATTCCACGCCCAGACCCTGGGAACCACCTACATCTA | | |
| | TGACTTCCCCGAAATGTTCACCCAGGCAAGTCCGGCGGCTCACACGCGGGTACATGTG | | |
| | CACAATGTGCAGCCTCTCTTTAAACTGTGGGGCTCCCCAGACAAGTATCCCAAACACA | | |
| | TCCTGACATACACTCAATTAGTGTTGGACTCTCAGGCCCAGCTGGTCCAGATGAACCG | | |
| | ACTTCCTGGTGGAAATGAGGTGCCCATGGTGGCCTTCAAAATGAGCTTTAAGACCCAG | | |
| | GAGTACCCGGAAGGACGGGATGTGATCGTCATCGGCAATGACATCACCTTTCGCATTG | | |
| | GATCCTTTGGCCCTGGAGAGCACCTTCTGTACCTGCCGGCATCCGAGATCCCCCGCGC | | |
| | AGAGGGCATTCCCAAAATTTACGTGGCAGCCAACAGTGGCGCCCGTATTCGCATGGCA | | |
| | GAGGAGATCAAACACATGTTCCACGTGGCTTGGGTGGACCCAGAAGACCCCCACAAAA | | |
| | AAAAAAAAACAGTGGCTTTCAGTGCAGGGAACTGGATTCGTAGCCTCACTAAAGTATT | | |
| | TTTTAAGGGATTTAAATACCTGTACCTGACTCCCCAAGACTACACCACAATCAGCTCC | | |
| | CTGAACTCCGTCCACTGTAAACACATCGAGGAAGGAGGAGAGTCCAGATACATGATCA | | |
| | CGGATATCATCGGGAAGGATGATGGCTTGGGCGTGGAGAATCTGAGGGGCTCAGGCAT | | |
| | GATTGCTGGGGAGTCCTCTCTGGCTTACGAAGAGATCGTCACCATTAGCTTGGTGACC | | |
| | TGCCGAGCCATTGGGATTGGGGCCTACTTGGTGAGGCTGGGCCAGCGAGTGATCCAGG | | |
| | TGGAGAATTCCCACATCATCCTCACAGGAGCAAGTGCTCTCAACAAGGTCCTGGGAAG | | |
| | AGAGGTCTACACATCCAACAACCAGCTGGGTGGCGTTCAGATCATGCATTACAATGGT | | |
| | GTCTCCCACATCACCGTGCCAGATGACTTTGAGGGGGTTTATACCATCCTGGAGTGGC | | |
| | TGTCCTATATGCCAAAGGATAATCACAGCCCTGTCCCTATCATCACACCCACTGACCC | | |
| | CATTGACAGAGAAATTGAATTCCTCCCATCCAGAGCTCCCTACGACCCCCGGTGGATG | | |
| | CTTGCAGGAAGGCCTCACCCAACTCTGAAGGGAACGTGGCAGAGCGGATTCTTTGACC | | |
| | ACGGCAGTTTCAAGGAAATCATGGCACCCTGGGCGCAGACCGTGGTGACAGGACGAGC | | |
| | AAGGCTTGGGGGGATTCCCGTGGGAGTGATTGCTGTGGAGACACGGACTGTGGAGGTG | | |
| | GCAGTCCCTGCACACCCTGCCAACCTGGATTCTGAGGCCAAGATAATTCAGCAGGCAG | | |
| | GACAGGTGTGGTTCCCAGACTCAGCCTACAAAACCGCCCAGGCCCTCAAGGACTTCAA | | |
| | CCGGGAGAAGTTCCCCCTGATGATCTTTGCCAACTGGAGGGCGTTCTCCGGTGGCATG | | |
| | AAAGACATGTATGACCAGGTGCTGAAGTTTGGAGCCTACATCGTGGACGGCCTTAGAC | | |
| | AATACAAACAGCCCATCCTGATCTATATCCCGCCCTATGCGGAGCTCCGGGGAGGCTC | | |
| | CTGGGTGGTCATAGATGCCACCATCAACCCGCTGTGCATAGAAATGTATGCAGACAAA | | |
| | GAGAGCAGGGGTGGTGTTCTGGAACCAGAGGGGACAGTGGAGATTAAGTTCCGAAAGA | | |
| | AAGATCTGATAAAGTCCATGAGAAGGATCGATCCAGCTTACAAGAAGCTCATGGAACA | | |
| | GCTAGGGGAACCTGATCTCTCCGACAAGGACCGAAAGGACCTGGAGGGCCGGCTAAAG | | |
| | GCTCGCGAGGACCTGCTGCTCCCCATCTACCACCAGGTGGCGGTGCAGTTCGCCGACT | | |
| | TCCATGACACACCCGGCCGGATGCTGGAGAAGGGCGTCATATCTGACATCCTGGAGTG | | |
| | GAAGACCGCACGCACCTTCCTGTATTGGCGTCTGCGCCGCCTCCTCCTGGAGGACCAG | | |
| | GTCAAGCAGGAGATCCTGCAGGCCAGCGGGGAGCTGAGTCACGTGCATATCCAGTCCA | | |
| | TGCTGCGTCGCTGGTTCGTGGAGACGGAGGGGGCTGTCAAGGCCTACTTGTGGGACAA | | |
| | CAACCAGGTGGTTGTGCAGTGGCTGGAACAGCACTGGCAGGCAGGGGATGGCCCGCGC | | |
| | TCCACCATCCGTGAGAACATCACGTACCTGAAGCACGACTCTGTCCTCAAGACCATCC | | |
| | GAGGCCTGGTTGAAGAAAACCCCGAGGTGGCCGTGGACTGTGTGATATACCTGAGCCA | | |
| | GCACATCAGCCCAGCTGAGCGGGCGCAGGTCGTTCACCTGCTGTCTACCATGGACAGC | | |
| | CCGGCCTCCACCTGA | | |
| | ORF Start: ATG at 1 | | ORF Stop: TGA at 7495 |
| | SEQ ID NO:218 | 2498 aa | MW at 280484.4 kD |

| | |
|---|---|
| NOV76a, | MVLLLCLSCLIFSCLTFSWLKIWGKMTDSKPITKSKSEANLIPSQEPFPASDNSGETP |
| CG59641-01 Protein Sequence | |
| | QRNGEGHTLPKTPSQAEPASHKGPKDAGRRRNSLPPSHQKPPRNPLSSSDAAPSPELQ |
| | ANGTGTQGLEATDTNGLSSSARPQGQQAGSPSKEDKKQANIKRQLMTNFILGSFDDYS |
| | SDEDSVAGSSRESTRKGSRASLGALSLEAYLTTRPSMSGLHLVKRGREHKKLDLHRDF |
| | TVASPAEFVTRFGGDRVIEKVLIANNGIAAVKCMRSIRRWAYEMFRNERAIRFVVMVT |
| | PEDLKANAEYIKMADHYVPVPGGPNNNNYANVELIVDIAKRIPVQAVWAGWGHASENP |
| | KLPELLCKNGVAFLGPPSEAMWALGDKIASTVVAQTLQVPTLPWSGSGLTVEWTEDDL |
| | QQGKRISVPEDVYDKGCVKDVDEGLEAAERTGFPLMIKASEGGGGKGIRKAESAEDFP |
| | ILFRQVQSEIPGSPIFLMKLAQHARHLEVQILADQYGNAVSLFGRDCSIQRRHQKIVE |
| | EAPATIAPLAIFEFMEQCAIRLAKTVGYVSAGTVEYLYSQDGSFHFLELNPRLQVEHP |
| | CTEMIADVNLPAAQLQIAMGVPLHRLKDIRLLYGESPWGVTPISFETPSNPPLARGBV |
| | IAARITSENPDEGFKPSSGTVQELNFRSSKNVWGYFSVAATGGLHEFADSQFGHCFSW |
| | GENREEAISNMVVALKELSIRGDFRTTVEYLINLLETESFQNNDIDTGWLDYLIAEKV |
| | QAEKPDIMLGVVCGALNVADAMFRTCMTDFLHSLERGQVLPADSLLNLVDVELIYGGV |
| | KYILKVARQSLTMFVLIMNGCHIEIDAHRLNDGGLLLSYNGNSYTTYMKEEVDSYRIT |
| | IGNKTCVFEKENDPTVLRSPSAGKLTQYTVEDGGHVEAGSSYAEMEVMKMIMTLNVQE |
| | RGRVKYIKRPGAVLEAGCVVARLELDDPSKVHPAEPFTGELPAQQTLPILGEKLHQVF |
| | HSVLENLTNVMSGFCLPEPVFSIKLKEWVQKLMMTLRHPSLPLLELQEIMTSVAGRIP |
| | APVEKSVRRVMAQYASNITSVLCQFPSQQIATILDCKAATLQRKADREVFFINTQSIV |
| | QLVQRYRSGIRCYMKTVVLDLLRRYLRVESKARDADANTSGMVGGVRSLSFTSVWCFV |
| | SPESHYDKCVINLREQFKPDMSQVLDCIFSHAQVAKKNQLVIMLIDELCGPDPSLSDE |
| | LISILNELTQLSKSEECKVALRARQILIASHLPSYELRHNQVESIFLSAIDMYGHQFC |
| | PENLKKLILSETTIFDVLPTFFYHANKVVCMASLEVYVRRGYIAYELNSLQHRQLPDG |
| | TCVVEFQFMLPSSEPNRMTVPISITNPDLLRHSTELFMDSGFSPLCQRNGANVAFRRF |
| | EDFTRNFDEVISCFANVPKDTPLFSEARTSLYSEDDCKSLREEPIHILNVSIQCADHL |
| | EDEALVPILRTFVQSKKNILVDYGLRRITFLIAQEFAEDRIYRHLEPALAFQLELNRN |
| | RNFDLTAVPCANHKMHLYLGAAKVKEGVEVTDHRFFIRAIIRHSDLITKEASFEYLQN |
| | ECERLLLEAMDELEVAFNNTSVRTDCNBIFLNFVPTVIMDPFKIEESVRYMVMRYGSR |
| | LWKLRVLQAEVKINIRQTTTGSAVPIRLPITNESGYYLDISLYKEVTDSRSGNIMFHS |
| | FGNKQGPQHGMLINTPYVTKDLLQAKRFQAQTLGTTYIYDfPEMFRQASPAAQTRVHV |
| | HNVQALFKLWGSPDKYPKDILTYTELVLDSQGQLVEMNRLPGGNEVGMVAFKMRFKTQ |
| | EYPEGRDVIVIGNDITFRIGSFGPGEDLLYLRASEMARAEGIPKIYVAANSGARIGMA |
| | EEIKHMFHVAWVDPEDPHKKKKTVAFSAGNWIRSLTKVPFKGFKYLYLTPQDYTRISS |
| | LNSVHCKHIEEGGESRYMITDIIGKDDGLGVENLRGSGMIAGESSLAYEEIVTISLVT |
| | CRAIGIGAYLVRLGQRVIQVENSHIILTGASALNKVLCREVYTSNNQLCGVQIMHYNG |
| | VSHITVPDDFEGVYTILEWLSYMPKDNESPVPIITPTDPIDREIEFLPSRAPYDPRWM |
| | LAGRPHPTLKGTWQSGPEDEGSFKEIMAPWAQTVVTGRARLGGIPVGVIAVETRTVEV |
| | AVPADPANLDSEAKIIQQAGQVWFPDSAYKTAQAVKDFNREKLPLMIFANWRGFSGGM |
| | KDMYDQVLKPGAYIVDGLRQYKQPILIYIPPYAELRGGSWVVIDATINPLCIEMYADK |
| | ESRGGVLEPEGTVEIKFRKKDLIKSMRRIDPAYKKLMEQLGEPDLSDKDRKDLEGRLK |
| | AREDLLLPIYHQVAVQFADFHDTPGRMLEKGVISDILEWKTARTFLYWRLRRLLLEDQ |
| | VKQEILQASGELSHVHIQSMLRRWFVETEGAVKAYLWDNNQVVVQWLEQHWQAGDGPR |
| | STIRENITYLKHDSVLKTIRGLVEENPEVAVDCVIYLSQHISPAERAQVVHLLSTMDS |
| | PAST |

Further analysis of the NOV76a protein yielded the following properties shown in Table 76B.

TABLE 76B
Protein Sequence Properties NOV76a

| | |
|---|---|
| PSort | 0.6850 probability located in endoplasmic reticulum |
| analysis: | (membrane); 0.6400 probability located in plasma |
| | membrane; 0.4600 probability located in Golgi |
| | body; 0.1000 probability located in endoplasmic |
| | reticulum (lumen) |
| SignalP | Likely cleavage site between residues 25 and 26 |
| analysis: | |

A search of the NOV76a protein against the Geneseq database, a proprietary database that contains sequences published in patents and patent publication, yielded several homologous proteins shown in Table 76C.

TABLE 76C
Geneseq Results for NOV76a

| | | NOV76a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU32848 | Novel human secreted protein | 26 . . . 2498 | 2316/2555 (90%) | 0.0 |
| | #3339-Homo sapiens, 2486 aa. | 1 . . . 2486 | 2339/2555 (90%) | |
| | [WO200179449-A2, | | | |
| | 25 OCT. 2001] | | | |
| AAR05707 | Acetyl-CoA-carboxylase-Gallus | 163 . . . 2498 | 1728/2375 (72%) | 0.0 |
| | sp, 2324 aa. [JP02057179-A, | 17 . . . 2324 | 2003/2375 (83%) | |
| | 26 FEB. 1990] | | | |
| AAB86033 | Bovine acetyl-coenzyme A | 204 . . . 2497 | 1719/2342 (73%) | 0.0 |
| | carboxylase−alpha protein | 14 . . . 2288 | 1969/2342 (83%) | |
| | fragment-Bos taurus, 2288 aa. | | | |
| | [DE19946173-A1, 5 APR. 2001] | | | |
| AAR98811 | Erysiphe graminis acetyl | 235 . . . 2490 | 1045/2326 (44%) | 0.0 |
| | coenzyme A carboxylase- | 42 . . . 2271 | 1432/2326 (60%) | |
| | Erysiphe graminis f.sp.hordei, | | | |
| | 2273 aa. [FR2727129-A1, | | | |
| | 24 MAY 1996] | | | |
| AAY24150 | Candida albicans acetyl CoA | 239 . . . 2489 | 1015/2300 (44%) | 0.0 |
| | carboxylase-Candida albicans, | 88 . . . 2269 | 1396/2300 (60%) | |
| | 2270 aa. [WO9932635-A1, | | | |
| | 1 JUL. 1999] | | | |

In a BLAST search of public sequence databases, the NOV76a protein was found to have homology to the proteins shown in the BLASTP data in Table 76D.

TABLE 76D
Public BLASTP Results for NOV76a

| | | NOV76a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O00763 | Acetyl-CoA carboxylase 2 (EC | 1 . . . 2498 | 2349/2528 (92%) | 0.0 |
| | 6.4.1.2) (ACC-beta) [Includes: | 1 . . . 2483 | 2384/2528 (93%) | |
| | Biotin carboxylase (BC 6.3.4.14)]- | | | |
| | Homo sapiens (Human), 2483 aa. | | | |
| O70151 | ACETYL-COA CARBOXYLASE- | 1 . . . 2497 | 2068/2524 (81%) | 0.0 |
| | Rattus norvegicus (Rat), 2456 aa. | 1 . . . 2455 | 2224/2524 (87%) | |
| CAA48770 | ACETYL-COA CARBOXYLASE | 163 . . . 2498 | 1921/2390 (80%) | 0.0 |
| | (EC 6.4.1.2)-Homo sapiens | 17 . . . 2339 | 2086/2390 (86%) | |
| | (Human), 2339 aa. | | | |
| P11029 | Acetyl-CoA carboxylase (EC | 163 . . . 2498 | 1732/2375 (72%) | 0.0 |
| | 6.4.1.2) (ACC) [Includes: Biotin | 17 . . . 2324 | 2004/2375 (83%) | |
| | carboxylase (EC 6.3.4.14)]- | | | |
| | Gallus gallus (Chicken), 2324 aa. | | | |
| P11497 | Acetyl-CoA carboxylase 1 (EC | 163 . . . 2497 | 1736/2396 (72%) | 0.0 |
| | 6.4.1.2) (ACC-alpha) [Includes: | 17 . . . 2345 | 1993/2396 (82%) | |
| | Biotin carboxylase (BC 6.3.4.14)]- | | | |
| | Rattus norvegicus (Rat), 2345 aa. | | | |

PFam analysis predicts that the NOV76a protein contains the domains shown in the Table 76E.

TABLE 76E
Domain Analysis of NOV76a

| | | Identities/ | |
|---|---|---|---|
| Pfam | NOV76a | Similarities for | Expect |
| Domain | Match Region | the Matched Region | Value |
| CPSase_L_chain: | 249 . . . 372 | 49/132 (37%) | 2.2e−57 |
| domain 1 of 1 | | | |
| CPSase_L_D2: | 374 . . . 619 | 102/253 (40%) | 6.6e−118 |
| domain 1 of 1 | | 218/253 (86%) | |
| Biotin_carb_C: | 640 . . . 747 | 40/118 (34%) | 1.9e−53 |
| domain 1 of 1 | | 100/118 (85%) | |
| biotin_lipoyl: | 885 . . . 951 | 22/75 (29%) | 6.5e−17 |
| domain 1 of 1 | | 56/75 (75%) | |
| Carboxyl_trans: | 1783 . . . 1878 | 31/100 (31%) | 7.4e−34 |
| domain 1 of 2 | | 88/100 (88%) | |
| GTP_cyclohydroI: | 2287 . . . 2304 | 6/18 (33%) | 6.6 |
| domain 1 of 1 | | 13/18 (72%) | |
| Carboxyl_trans: | 1897 . . . 2374 | 191/504 (38%) | 4.1e−258 |
| domain 2 of 2 | | 447/504 (89%) | |

### Example 77

The NOV77 clone was analyzed, and the nucleotide and predicted polypeptide sequences are shown in Table 77A.

TABLE 77A
NOV77 Sequence Analysis

| | |
|---|---|
| SEQ ID NO: 219 | 1624 bp |

| | |
|---|---|
| NOV77a, | CGCGCGCCGGGGATGGAGCCGCAGCCCGGCGGCGCCCGGAGCTGCCGGCGCGGGGCCC |
| CG59630-01 DNA Sequence | |
| | CCGGCGGCGCCTGCGAGCTGGGCCCGGCGGCCGAGGCGGCGCCCATGAGCCTCGCCAT |
| | CCACAGCACCACGGGCACCCGCTACGACCTGGCCGTGCCGCCCGACGAGACGGTGGAG |
| | GGGCTGCGCAAGCGGTTGTCCCAGCGCCTCAAAGTGCCCAAGGAGCGCCTGGCTCTTC |
| | TCCACAAAGACACGCGGCTCAGTTCGGGGAAGCTGCAGGAGTTCGGCGTGGGTGATGG |
| | CAGCAAGCTGACCTTGGTACCCACCGTGGAAGCGGGCCTCATGTCTCAGGCCTCAAGG |
| | CCGGAACAGTCCGTGATGCAAGCTCTCGAGAGTCTCACGGAGACGCAGCCCCCAGCGG |
| | CGCCCGGGCCGGGCCGGGCTGGCGGAGGAGGCTTCCGGAAATACAGATTCATTTTATT |
| | TAAGCGTCCGTGGCACCGACAGGGACCCCAGAGCCCAGAGAGGGGCGGCGAGAGGCCC |
| | CAGGTCAGTGACTTCCTGTCGGGCCGTTCGCCACTGACACTGGCCTTGCGTGTGGGCG |
| | ACCACATGATGTTCGTGCAGCTGCAGCTCGCGGCCCAGCACGCTCCACTGCAACACCG |
| | CCATGTGCTGGCCGCTGCGGCCGCCGCCGCTGCTGCGCGGGGGGACCCCAGCATAGCC |
| | TCCCCCGTGTCCTCGCCCTGCCGGCCGGTGTCCAGTGCCGCCCGAGTCCCCCCGGTGC |
| | CCACCAGCCCGTCCCCTGCATCTCCCTCGCCCATCACAGCCGGCTCCTTCCGGTCCCA |
| | CGCAGCCTCCACCACCTGCCCGGAGCAGATGGACTGCTCCCCCACGGCCAGCAGCAGT |
| | GCCAGTCCTGGTGCCAGCACCACGTCTACCCCAGGGGCCAGCCCTGCCCCCCGCTCCC |
| | GAAAACCCGGCGCCGTCATCGAGAGCTTTGTGAATCACGCCCCGGGGGTCTTCTCAGG |
| | GACCTTCTCTGGCACGCTACACCCCAACTGCCAAGACAGCAGCGGGCGGCCGCGGCGT |
| | GACATCGGCACCATCCTGCAGATCCTGAACGACCTCCTGAGCGCCACCCGGCACTACC |
| | AGGGCATGCCCCCTTCGCTGGCCCAGCTCCGCTGCCACGCCCAGTGCTCCCCGGCCTC |
| | ACCGGCCCCCGACCTGGCCCCCAGAACTACCTCCTGCGAGAAGCTCACGGCTGCCCCC |
| | TCAGCCTCCCTGCTGCAGGGCCAGAGCCAGATCCGCATGTGCAAGCCCCCGGGTGACC |
| | GGCTTCGGCAGACAGAAAACCGCGCCACGCGCTGCAAGGTGGAACGGCTGCAGCTGCT |
| | TCTGCAGCAGAAACGGCTCCGTAGAAAGGCCCGGCGGGACGCGCGGGGTCCGTACCAC |
| | TGGTCACCCAGCCGCAAGGCCGGCCGCAGCGACAGCAGTAGCAGCGGGGGCGGCGGCA |
| | GCCCCAGCGAGGCCTCCGGCTTGGGCCTCGACTTCGAGGACTCCGTGTGGAAGCCAGA |
| | AGTCAACCCTGACATCAAGTCAGAGTTCGTGGTGGCTTAGGATCTTCGGATCGGCCAC |
| | CCTCGCCCCTCGCACCCCAGCCCAGGGCGGCGGGGACTCCGAGAGCCCCGGAGAGAAC |

| | |
|---|---|
| ORF Start: ATG at 13 | ORF Stop: TAG at 1546 |

| | | |
|---|---|---|
| SEQ ID NO: 220 | 511 aa | MW at 53949.3 kD |

| | |
|---|---|
| NOV77a, | MEPQPGGARSCRRGAPGGACELGPAAEAAPMSLAIHSTTGTRYDLAVPPDETVEGLRK |
| CG59630-01 Protein Sequence | |
| | RLSQRLKVPKERLALLHKDTRLSSGKLQEFGVGDGSKLTLVPTVEAGLMSQASRPEQS |
| | VMQALESLTETQPPAAPGPGRAGGGGFRKYRFILFKRPWHRQGPQSPERGGERPQVSD |
| | FLSGRSPLTLALRVGDHMMFVQLQLAAQHAPLQHRHVLAAAAAAAAARGDPSIASPVS |
| | SPCRPVSSAARVPPVPTSPSPASPSPITAGSFRSHAASTTCPEQMDCSPTASSSASPG |
| | ASTTSTPGASPAPRSRKPGAVIESFVNHAPGVFSGTFSGTLHPNCQDSSGRPRRDIGT |
| | ILQILNDLLSATRHYQGMPPSLAQLRCHAQCSPASPAPDLAPRTTSCEKLTAAPSASL |
| | LQGQSQIRMCKPPGDRLRQTENRATRCKVERLQLLLQQKRLRRKARRDARGPYHWSPS |
| | RKAGRSDSSSSGGGGSPSEASGLGLDFEDSVWKPEVNPDIKSEFVVA |

Further analysis of the NOV77a protein yielded the following properties shown in Table 77B.

TABLE 77B
Protein Sequence Properties NOV77a

| | |
|---|---|
| PSort | 0.3000 probability located in microbody (peroxisome); |
| analysis: | 0.3000 probability located in nucleus; 0.1526 probability |
| | located in lysosome (lumen); 0.1000 probability located in |
| | mitochondrial matrix space |
| SignalP | No Known Signal Sequence Predicted |
| analysis: | |

A search of the NOV77a protein against the Geneseq database, a proprietary database that contains sequences published in patents and patent publication, yielded several homologous proteins shown in Table 77C.

TABLE 77C
Geneseq Results for NOV77a

| | | NOV77a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB56832 | Human prostate cancer antigen | 267 . . . 493 | 189/227 (83%) | e−104 |
| | protein sequence SEQ ID NO: 1410- | 1 . . . 227 | 195/227 (85%) | |
| | Homo sapiens, 236 aa. | | | |
| | [WO200055174-A1, 21 SEP. 2000] | | | |

In a BLAST search of public sequence databases, the NOV77a protein was found to have homology to the proteins shown in the BLASTP data in Table 77D.

TABLE 77D
Public BLASTP Results for NOV77a

| | | NOV77a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9JJJ6 | MIDNOLIN-Mus musculus | 1 . . . 511 | 475/514 (92%) | 0.0 |
| | (Mouse), 508 aa. | 1 . . . 508 | 486/514 (94%) | |
| Q96BW8 | SIMILAR TO MIDNOLIN- | 338 . . . 511 | 174/174 (100%) | 2e−97 |
| | Homo sapiens (Human), 177 aa | 4 . . . 177 | 174/174 (100%) | |
| | (fragment). | | | |
| Q9W2S4 | CG9732 PROTEIN-Drosophila | 213 . . . 363 | 58/155 (37%) | 6e−18 |
| | melanogaster (Fruit fly), 989 aa. | 524 . . . 677 | 80/155 (51%) | |
| AAL40834 | BPLF1-Human herpesvirus 4 | 200 . . . 406 | 64/223 (28%) | 2e−07 |
| | (Epstein-Barr virus), 3179 aa. | 320 . . . 530 | 95/223 (41%) | |
| Q9BKV7 | PPG3-Leishmania major, 1325 | 213 . . . 328 | 37/121 (30%) | 2e−06 |
| | aa. | 984 . . . 1104 | 66/121 (53%) | |

PFam analysis predicts that the NOV77a protein contains the domains shown in the Table 77E.

TABLE 77E
Domain Analysis of NOV77a

| | | Identities/ | |
|---|---|---|---|
| Pfam | NOV77a | Similarities for | Expect |
| Domain | Match Region | the Matched Region | Value |
| ubiquitin: | 31 . . . 99 | 19/79 (24%) | 0.00033 |
| domain 1 of 1 | | 46/79 (58%) | |
| PI3_PI4_kinase: | 411 . . . 427 | 7/18 (39%) | 1.5 |
| domain 1 of 1 | | 14/18 (78%) | |

### Example 78

The NOV78 clone was analyzed, and the nucleotide and predicted polypeptide sequences are shown in Table 78A.

TABLE 78A
NOV78 Sequence Analysis

| | |
|---|---|
| SEQ ID NO: 221 | 1034 bp |

| | |
|---|---|
| NOV78a | CCACCGCCACAGCTGCCAGCATGTCTGGCCCAGACATCAAGACGCCGACCGCCATCCA |
| CG59561-01 DNA Sequence | |
| | GATCTGCCGGATTATGCGGGACGCTAATGTGGCCCGCAATGTCTACGGCGGGACCATC |
| | CTGAAGATGATCAAAGAGGCGGGCGCCATCATCAGCACCCGGCATTGCAATCCGCAGA |
| | ACGGGGATCGCTGTGTGGCCGCTCTGGCTCGGGTCGAGTGCACCCACTTCCTGTGGCC |
| | CATGTGCATCGGTGAGGTGGCCCACGTCAGCGCGGAGATCACCTACACCTCCAAGCAC |
| | TCTGTGGAGGTGCAGGTCAACATGATGTCCGAAAACATCCTCACAGGTGCCAAAAAGC |
| | TGACCAATAAGGCCACCCTCTGGTATGCGCCCCTGTCGCTGACGAACGTGGACAAGGT |
| | CCTCGAAGAGCCTCCTGTTGTGTATTTCCGGCAGGAGCAGGAGGAGGAGGGCCAGAAG |
| | CGGTACAAAACCCAGAAGCTGGAGCGCATGGAGACCAACTGGAGGAACGGGGACATCG |
| | TCCAGCCAGTCCTCAACCCAGAGCCGAACACTGTCAGCTACAGCCAGTCCAGCTTGAT |
| | CCACCTGGTGGGGCCTTCAGACTGTACCCTGCACAGCTTCGTGCATGAAGGGGTGACC |
| | ATGAAGGTCATGGACGAGGTCGCCGGGATCTTGGCTGCACGCCACTGCAAGACCAACC |
| | TCGTCACAGCCTCCATGGAGGCCATTAATTTTGACAACAAGATCAGAAAAGGCTGCAT |
| | CAAGACCATCTCCGGACGCATGACCTTCACGAGCAATAAGTCCGTAGAGATCGAGGTC |
| | TTGGTGGATGCCGACTGTGTTGTGGACAGCTCTCAGAAGCGCTACAGGGCCGCCAGTG |
| | TCTTCACCTATGTGTCGCTGAGCCAGGAAGGCAGGTCGCTGCCCATGCCCCAGCTCGT |
| | GCCGGAGACCCAGGACGAGAAGGGCTTTGAGGCCTGGCTCGGTGGCTCACGCCTATAA |
| | TCCCAGCACTTTAGGATGCTGAGGCAGGCGGATCACTTGACGTCAGGA |

| | |
|---|---|
| ORF Start: ATG at 21 | ORF Stop: TAA at 984 |

| | | |
|---|---|---|
| SEQ ID NO: 222 | 321 aa | MW at 35738.7 kD |

| | |
|---|---|
| NOV78a, | MSGPDIKTPTAIQICRIMRDANVARNVYGGTILKMIKEAGAIISTRHCNPQNGDRCVA |
| CG59561-01 Protein Sequence | |
| | ALARVECTHFLWPMCIGEVAHVSAEITYTSKHSVEVQVNMMSENILTGAKKLTNKATL |
| | WYAPLSLTNVDKVLEEPPVVYFRQEQEEEGQKRYKTQKLERMETNWRNGDIVQPVLNP |
| | EPNTVSYSQSSLIHLVGPSDCTLHSFVHEGVTMKVMDEVAGILAARHCKTNLVTASME |
| | AINFDNKIRKGCIKTISGRMTFTSNKSVEIEVLVDADCVVDSSQKRYRAASVFTYVSL |
| | SQEGRSLPMPQLVPETQDEKGFEAWLGGSRL |

Further analysis of the NOV78a protein yielded the following properties shown in Table 78B.

TABLE 78B
Protein Sequence Properties NOV78a

| | |
|---|---|
| PSort | 0.8000 probability located in microbody (peroxisome); |
| analysis: | 0.1000 probability located in mitochondrial matrix space; |
| | 0.1000 probability located in lysosome (lumen); 0.0000 |
| | probability located in endoplasmic reticulum (membrane) |
| SignalP | No Known Signal Sequence Predicted |
| analysis: | |

A search of the NOV78a protein against the Geneseq database, a proprietary database that contains sequences published in patents and patent publication, yielded several homologous proteins shown in Table 78C.

TABLE 78c
Geneseq Results for NOV78a

| | | NOV78a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAW74896 | Human secreted protein encoded by | 1 . . . 310 | 273/313 (87%) | e−154 |
| | gene 169 clone HPTTU11-Homo | 1 . . . 313 | 292/313 (93%) | |
| | sapiens, 339 aa. [WO9839448-A2, | | | |
| | 11 SEP. 1998] | | | |
| AAY71115 | Human Hydrolase protein-13 | 1 . . . 310 | 247/313 (78%) | e−133 |
| | (HYDRL-13)-Homo sapiens, 375 | 33 . . . 316 | 266/313 (84%) | |
| | aa. [WO200028045-A2, 18 MAY 2000] | | | |
| AAY35275 | Chlamydia pneumoniae | 187 . . . 310 | 35/124 (28%) | 1e−09 |
| | transmembrane protein sequence- | 16 . . . 138 | 72/124 (57%) | |
| | Chlamydia pneumoniae, 155 aa. | | | |
| | [WO9927105-A2, 3 JUN. 1999] | | | |
| AAG92590 | C glutamicum protein fragment | 24 . . . 309 | 69/296 (23%) | 7e−08 |
| | SEQ ID NO: 6344- | 35 . . . 307 | 112/296 (37%) | |
| | Corynebacterium glutamicum, 339 | | | |
| | aa. [EP1108790-A2, 20 JUN. 2001] | | | |
| AAB76624 | Corynebacterium glutamicum MCT | 24 . . . 309 | 69/296 (23%) | 7e−08 |
| | protein SEQ ID NO: 230- | 35 . . . 307 | 112/296 (37%) | |
| | Corynebacterium glutamicum, 339 | | | |
| | aa. [WO200100805-A2, 4 JAN. 2001] | | | |

In a BLAST search of public sequence databases, the NOV78a protein was found to have homology to the proteins shown in the BLASTP data in Table 78D.

TABLE 78D
Public BLASTP Results for NOV78a

| | | NOV78a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O00154 | Cytosolic acyl coenzyme A thioester | 1 . . . 310 | 274/313 (87%) | e−154 |
| | hydrolase (EC 3.1.2.2) (Long chain | 1 . . . 313 | 293/313 (93%) | |
| | acyl-CoA thioester hydrolase) (CTE- | | | |
| | II) (Brain acyl-CoA hydrolase) | | | |
| | (BACH) - Homo sapiens (Human), | | | |
| | 338 aa. | | | |
| Q91V12 | ACYL-COA HYDROLASE | 1 . . . 310 | 265/313 (84%) | e−150 |
| | (HYPOTHETICAL 37.6 KDA | 1 . . . 313 | 287/313 (91%) | |
| | PROTEIN) - Mus musculus (Mouse), | | | |
| | 338 aa. | | | |
| Q64559 | Cytosolic acyl coenzyme A thioester | 1 . . . 310 | 263/313 (84%) | e−149 |
| | hydrolase (EC 3.1.2.2) (Long chain | 1 . . . 313 | 286/313 (91%) | |
| | acyl-CoA thioester hydrolase) (CTE- | | | |
| | II) (Brain acyl-CoA hydrolase) | | | |
| | (BACH) (ACT) (LACH1) (ACH1) - | | | |
| | Rattus norvegicus (Rat), 338 aa. | | | |
| JC5416 | palmitoyl-CoA hydrolase (EC | 12 . . . 310 | 251/302 (83%) | e−142 |
| | 3.1.2.2), hepatic - rat, 343 aa. | 17 . . . 318 | 276/302 (91%) | |
| Q9Y541 | DJ202O8.3.1 (HBACH (BRAIN | 1 . . . 202 | 181/204 (88%) | e−100 |
| | ACYL-COA HYDROLASE (ACYL | 33 . . . 236 | 190/204 (92%) | |
| | COENZYME A THIOESTER | | | |
| | HYDROLASE, EC 3.1.2.2)) | | | |
| | (ISOFORM 1)) - Homo sapiens, | | | |
| | (Human), 237 aa (fragment). | | | |

PFam analysis predicts that the NOV78a protein contains the domains shown in the Table 78E.

TABLE 78E
Domain Analysis of NOV78a

| | | Identities/ | | |
|---|---|---|---|---|
| | | Similarities | | |
| | NOV78a Match | for the Matched | | Expect |
| Pfam Domain | Region | Region | | Value |
| Acyl-CoA_hydro: | 165 . . . 305 | 46/147 | (31%) | 1.1e−47 |
| domain 1 of 1 | | 131/147 | (89%) | |

The NOV79 clone was analyzed, and the nucleotide and predicted polypeptide sequences are shown in Table 79A.

TABLE 79A
NOV79 Sequence Analysis

| | |
|---|---|
| SEQ ID NO: 223 | 4203 bp |

| | |
|---|---|
| NOV79a | AATGTGATGGGATCACTAGCATGTCTGCGGAGAGCGGCCCTGGGACGAGATTGAGAAA |
| CG59452-01 DNA Sequence | |
| | TCTGCCAGTAATGGGGGATGGACTAGAAACTTCCCAAATGTCTACAACACAGGCCCAG |
| | GCCCAACCCCAGCCAGCCAACGCAGCCAGCACCAACCCCCCGCCCCCAGAGACCTCCA |
| | ACCCTAACAAGCCCAAGAGGCAGACCAACCAACTGCAATACCTGCTCAGAGTGGTGCT |
| | CAAGACACTATGGAAACACCAGTTTGCATGGCCTTTCCAGCAGCCTGTGGATGCCGTC |
| | AAGCTGAACCTCCCTGATTACTATAAGATCATTAAAACGCCTATGGATATGGGAACAA |
| | TAAAGAAGCGCTTGGAAAACAACTATTACTGGAATGCTCAGGAATGTATCCAGGACTT |
| | CAACACTATGTTTACAAATTGTTACATCTACAACAAGCCTGGAGATGACATAGTCTTA |
| | ATGGCAGAAGCTCTGGAAAAGCTCTTCTTGCAAAAAATAAATGAGCTACCCACAGAAG |
| | AAACCGAGATCATGATAGTCCAGGCAAAAGGAAGAGGACGTGGGAGGAAAGAAACAGG |
| | TACAGCAAAACCTGGCGTTTCCACGGTACCAAACACAACTCAAGCATCGACTCCTCCG |
| | CAGACCCAGACCCCTCAGCCGAATCCTCCTCCTGTGCAGGCCACGCCTCACCCCTTCC |
| | CTGCCGTCACCCCGGACCTCATCGTCCAGACCCCTGTCATGACAGTGGTGCCTCCCCA |
| | GCCACTGCAGACGCCCCCGCCAGTGCCCCCCCAGCCACAACCCCCACCCGCTCCAGCT |
| | CCCCAGCCCGTACAGAGCCACCCACCCATCATCGCGGCCACCCCACAGCCTGTGAAGA |
| | CAAAGAAGGGAGTGAAGAGGAAAGCAGACACCACCACCCCCACCACCATTGACCCCAT |
| | TCACGAGCCACCCTCGCTGCCCCCGGAGCCCAAGACCACCAAGCTGGGCCAGCGGCGG |
| | GAGAGCAGCCGGCCTGTGAAACCTCCAAAGAAGGACGTGCCCGACTCTCAGCAGCACC |
| | CAGCACCAGAGAAGAGCAGCAAGGTCTCGGAGCAGCTCAAGTGCTGCAGCGGCATCCT |
| | CAAGGAGATGTTTGCCAAGAAGCACGCCGCCTACGCCTGGCCCTTCTACAAGCCTGTG |
| | GACGTGGAGGCACTGGGCCTACACGACTACTGTGACATCATCAAGCACCCCATGGACA |
| | TGAGCACAATCAAGTCTAAACTGGAGGCCCGTGAGTACCGTGATGCTCAGGAGTTTGG |
| | TGCTGACGTCCGATTGATGTTCTCCAACTGCTATAAGTACAACCCTCCTGACCATGAG |
| | GTGGTGGCCATGGCCCGCAAGCTCCAGGATGTGTTCGAAATGCGCTTTGCCAAGATGC |
| | CGGACGAGCCTGAGGAGCCAGTGGTGGCCGTGTCCTCCCCGGCAGTGCCCCCTCCCAC |
| | CAAGGTTGTGGCCCCGCCCTCATCCAGCGACAGCAGCAGCGATAGCTCCTCGGACAGT |
| | GACAGTTCGACTGATGACTCTGAGGAGGAGCGAGCCCAGCGGCTGGCTGAGCTCCAGG |
| | AGCAGCTCAAAGCCGTGCACGAGCAGCTTGCAGCCCTCTCTCAGCCCCAGCAGAACAA |
| | ACCAAAGAAAAAGGAGAAAGACAAGAAGGAAAAGAAAAAAGAAAAGCACAAAAGGAAA |
| | GAGGAAGTGGAAGAGAATAAAAAAAGCAAAGCCAAGGAACCTCCTCCTAAAAAGACGA |
| | AGAAAAATAATAGCAGCAACAGCAATGTGAGCAAGAAGGAGCCAGCGCCCATGAAGAG |
| | CAAGCCCCCTCCCACGTATGAGTCGGAGGAAGAGGACAAGTGCAAGCCTATGTCCTAT |
| | GAGGAGAAGCGGCAGCTCAGCTTGGACATCAACAAGCTCCCCGGCGAGAAGCTGGGCC |
| | GCGTGGTGCACATCATCCAGTCACGGGAGCCCTCCCTGAAGAATTCCAACCCCGACGA |
| | GATTGAAATCGACTTTGAGACCCTGAAGCCGTCCACACTGCGTGAGCTGGAGCGCTAT |
| | GTCACCTCCTGTTTGCGGAAGAAAAGGAAACCTCAAGCTGAGAAAGTTGATGTGATTG |
| | CCGGCTCCTCCAAGATGAAGGGCTTCTCGTCCTCAGAGTCGGAGAGCTCCAGTGAGTC |
| | CAGCTCCTCTGACAGCGAAGACTCCGAAACAGAGATGGCTCCGAAGTCAAAAAAGAAG |
| | GGGCACCCCGGGAGGGAGCAGAAGCAGCACCATCATCACCACCATCAGCAGATGCAGC |
| | AGGCCCCGGCTCCTGTGCCCCAGCAGCCGCCCCCGCCTCCCCAGCAGCCCCCACCGCC |
| | TCCACCTCCGCAGCAGCAACAGCAGCCGCCACCCCCGCCTCCCCCACCCTCCATGCCG |
| | CAGCAGGCAGCCCCGGCGATGAAGTCCTCGCCCCCACCCTTCATTGCCACCCAGGTGC |
| | CCGTCCTGGAGCCCCAGCTCCCAGGCAGCGTCTTTGACCCCATCGGCCACTTCACCCA |
| | GCCCATCCTGCACCTGCCGCAGCCTGAGCTGCCCCCTCACCTGCCCCAGCCGCCTGAG |
| | CACAGCACTCCACCCCATCTCAACCAGCACGCAGTGGTCTCTCCTCCAGCTTTGCACA |
| | ACGCACTACCCCAGCAGCCATCACGGCCCAGCAACCGAGCCGCTGCCCTGCCTCCCAA |
| | GCCCGCCCGGCCCCCAGCCGTGTCACCAGCCTTGACCCAAACACCCCTGCTCCCACAG |
| | CCCCCCATGGCCCAACCCCCCCAAGTGCTGCTGGAGGATGAAGAGCCACCTGCCCCAC |
| | CCCTCACCTCCATGCAGATGCAGCTGTACCTGCAGCAGCTGCAGAAGGTGCAGCCCCC |
| | TACGCCGCTACTCCCTTCCGTGAAGGTGCAGTCCCAGCCCCCACCCCCCCTGCCGCCC |
| | CCACCCCACCCCTCTGTGCAGCAGCAGCTGCAGCAGCAGCCGCCACCACCCCCACCAC |
| | CCCAGCCCCAGCCTCCACCCCAGCAGCAGCATCAGCCCCCTCCACGGCCCGTGCACTT |
| | GCAGCCCATGCAGTTTTCCACCCACATCCAACAGCCCCCGCCACCCCAGGGCCAGCAG |
| | CCCCCCCATCCGCCCCCAGGCCAGCAGCCACCCCCGCCGCAGCCTGCCAAGCCTCAGC |
| | AAGTCATCCAGCACCACCATTCACCCCGGCACCACAAGTCGGACCCCTACTCAACCGG |
| | TCACCTCCGCGAAGCCCCCTCCCCGCTTATGATACATTCCCCCCAGATGTCACAGTTC |
| | CAGAGCCTGACCCACCAGTCTCCACCCCAGCAAAACGTCCAGCCTAAGAAACAGGTAA |
| | CTGGCAGGGCTGGGCCAAGTCCTGTGGGCCAGGGCCGGGGGTGCCTGCCCACCTCACC |
| | GGCCGCTGTGCCTGTGCCATCCCAGGAGCTGCGTGCTGCCTCCGTGGTCCAGCCCCAG |
| | CCCCTCGTGGTGGTGAAGGAGGAGAAGATCCACTCACCCATCATCCGCAGCGAGCCCT |
| | TCAGCCCCTCGCTGCGGCCGGAGCCCCCCAAGCACCCGGAGAGCATCAAGGCCCCCGT |
| | TTATGTTCCAGGGCCGGAAATGAAGCCTGTGGATGTCGGGAGGCCTGTGATCCGGCCC |
| | CCAGAGCAGAACGCACCGCCACCAGGGGCCCCTGACAAGGACAAACAGAAACAGGAGC |
| | CGAAGACTCCAGTTGCGCCCAAAAAGGACCTGAAAATCAAGAACATGGGCTCCTGGGC |
| | CAGCCTAGTGCAGAAGCATCCGACCACCCCCTCCTCCACAGCCAAGTCATCCAGCGAC |
| | AGCTTCGAGCAGTTCCGCCGCGCCGCTCGGGAGAAAGAGGAGCGTGAGAAGGCCCTGA |
| | AGGCTCAGGCCGAGCACGCTGAGAAGGAGAAGGAGCGGCTGCGGCAGGAGCGCATGAG |
| | GAGCCGAGAGGACGAGGATGCGCTGGAGCAGGCCCGGCGGGCCCATGAGGAGGCACGT |
| | CGGCGCCAGGAGCAGCAGCAGCAGCAGCGCCAGGAGCAACAGCAGCAGCAGCAACAGC |
| | AAGCAGCTGCGGTGGCTGCCGCCGCCACCCCACAGGCCCAGAGCTCCCAGCCCCAGTC |
| | CATGCTGGACCAGCAGAGGGAGTTGGCCCGGAAGCGGGAGCAGGAGCGAAGACGCCGG |
| | GAAGCCATGGCAGCTACCATTGACATGAATTTCCAGAGTGATCTATTGTCAATATTTG |
| | AAGAAAATCTTTTCTGAGCGCACCTAG |

| | |
|---|---|
| ORF Start: ATG at 21 | ORF Stop: TGA at 4191 |

| | | |
|---|---|---|
| SEQ ID NO: 224 | 1390 aa | MW at 154728.4 kD |

| | |
|---|---|
| NOV79a, | MSAESGPGTRLRNLPVMGDGLETSQMSTTQAQAQPQPANAASTNPPPPETSNPNKPKR |
| CG59452-01 Protein | |
| Sequences | QTNQLQYLLRVVLKTLWKHQFAWPFQQPVDAVKLNLPDYYKIIKTPMDMGTIKKRLEN |
| | NYYWNAQECIQDFNTMFTNCYIYNKPGDDIVLMAEALEKLFLQKINELPTEETEIMIV |
| | QAKGRGRGRKETGTAKPGVSTVPNTTQASTPPQTQTPQPNPPPVQATPHPFPAVTPDL |
| | IVQTPVMTVVPPQPLQTPPPVPPQPQPPPAPAPQPVQSHPPIIAATPQPVKTKKGVKR |
| | KADTTTPTTIDPIHEPPSLPPEPKTTKLGQRRESSRPVKPPKKDVPDSQQHPAPEKSS |
| | KVSEQLKCCSGILKEMFAKKHAAYAWPFYKPVDVEALGLHDYCDIIKHPMDMSTIKSK |
| | LEAREYRDAQEFGADVRLMFSNCYKYNPPDHEVVAMARKLQDVFEMRFAKMPDEPEEP |
| | VVAVSSPAVPPPTKVVAPPSSSDSSSDSSSDSDSSTDDSEEERAQRLAELQEQLKAVH |
| | EQLAALSQPQQNKPKKKEKDKKEKKKEKHKRKEEVEENKKSKAKEPPPKKTKKNNSSN |
| | SNVSKKEPAPMKSKPPPTYESEEEDKCKPMSYEEKRQLSLDINKLPGEKLGRVVHIIQ |
| | SREPSLKNSNPDEIEIDFETLKPSTLRELERYVTSCLRKKRKPQAEKVDVIAGSSKMK |
| | GFSSSESESSSESSSSDSEDSETEMAPKSKKKGHPGREQKQHHHHHHQQMQQAPAPVP |
| | QQPPPPPQQPPPPPPPQQQQQPPPPPPPPSMPQQAAPAMKSSPPPFIATQVPVLEPQL |
| | PGSVFDPIGHFTQPILHLPQPELPPHLPQPPEHSTPPHLNQHAVVSPPALHNALPQQP |
| | SRPSNRAAALPPKPARPPAVSPALTQTPLLPQPPMAQPPQVLLEDEEPPAPPLTSMQM |
| | QLYLQQLQKVQPPTPLLPSVKVQSQPPPPLPPPPHPSVQQQLQQQPPPPPPPQPQPPP |
| | QQQHQPPPRPVHLQPMQFSTHIQQPPPPQGQQPPHPPPGQQPPPPQPAKPQQVIQHHH |
| | SPRHHKSDPYSTGHLREAPSPLMIHSPQMSQFQSLTHQSPPQQNVQPKKQVTGRAGPS |
| | PVGQGRGCLPTSPAAVPVPSQELRAASVVQPQPLVVVKEEKIHSPIIRSEPFSPSLRP |
| | EPPKHPESIKAPVYVPGPEMKPVDVGRPVIRPPEQNAPPPGAPDKDKQKQEPKTPVAP |
| | KKDLKIKNMGSWASLVQKHPTTPSSTAKSSSDSFEQFRRAAREKEEREKALKAQAEHA |
| | EKEKERLRQERMRSREDEDALEQARRAHEEARRRQEQQQQQRQEQQQQQQQQAAAVAA |
| | AATPQAQSSQPQSMLDQQRELARKREQERRRREAMAATIDMNFQSDLLSIFEENLF |

Further analysis of the NOV79a protein yielded the following properties shown in Table 79B.

TABLE 79B
Protein Sequence Properties NOV79a

| | |
|---|---|
| PSort | 0.9800 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV79a protein against the Geneseq database, a proprietary database that contains sequences published in patents and patent publication, yielded several homologous proteins shown in Table 79C.

TABLE 79C
Geneseq Results for NOV79a

| | | NOV79a | Identities/ | |
|---|---|---|---|---|
| | | Residues/ | Similarities | |
| Geneseq | Protein/Organism/Length | Match | for the | Except |
| Identifier | [Patent #, Date] | Residues | Matched Region | Value |
| AAY57898 | Human transmembrane protein | 1 . . . 667 | 667/667 (100%) | 0.0 |
| | HTMPN-22 - Homo sapiens, 688 | 1 . . . 667 | 667/667 (100%) | |
| | aa. [WO9961471-A2, 2 DEC | | | |
| | 1999] | | | |
| AAY07027 | Breast cancer associated antigen | 44 . . . 724 | 407/732 (55%) | 0.0 |
| | precursor sequence - Homo | 13 . . . 708 | 487/732 (65%) | |
| | sapiens, 754 aa. [WO9904265-A2, | | | |
| | 28 JAN 1999] | | | |
| AAY07114 | WO9904265 Seq ID No: 685 - | 35 . . . 738 | 357/761 (46%) | e−170 |
| | Homo sapiens, 947 aa. | 4 . . . 686 | 444/761 (57%) | |
| | [WO9904265-A2, 28 JAN 1999] | | | |
| AAW81168 | Transcriptional regulatory factor | 35 . . . 738 | 357/761 (46%) | e−170 |
| | RING3 - Homo sapiens, 947 aa. | 4 . . . 686 | 444/761 (57%) | |
| | [WO9848015-A1, 29 OCT 1998] | | | |
| AAU16206 | Human novel secreted protein, Seq | 51 . . . 255 | 118/206 (57%) | 2e−59 |
| | ID 1159 - Homo sapiens, 235 aa. | 1 . . . 203 | 137/206 (66%) | |
| | [WO200155322-A2, 2 AUG | | | |
| | 2001] | | | |

In a BLAST search of public sequence databases, the NOV79a protein was found to have homology to the proteins shown in the BLASTP data in Table 79D.

TABLE 79D
Public BLASTP Results for NOV79a

| | | NOV79a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| O60885 | Bromodomain-containing protein | 1 . . . 1390 | 1357/1391 | (97%) | 0.0 |
| | 4 (HUNK1 protein) - Homo | 1 . . . 1362 | 1360/1391 | (97%) | |
| | sapiens (Human), 1362 aa. | | | | |
| Q9ESU6 | CELL PROLIFERATION | 1 . . . 1390 | 1318/1400 | (94%) | 0.0 |
| | RELATED PROTEIN CAP - | 1 . . . 1400 | 1338/1400 | (95%) | |
| | Mus musculus (Mouse), 1400 aa. | | | | |
| AAL67833 | BROMODOMAIN- | 1 . . . 1390 | 1318/1400 | (94%) | 0.0 |
| | CONTAINING PROTEIN BRD4 | 1 . . . 1400 | 1338/1400 | (95%) | |
| | LONG VARIANT - Mus | | | | |
| | musculus (Mouse), 1400 aa. | | | | |
| O60433 | R31546_1 - Homo sapiens | 1 . . . 719 | 719/719 | (100%) | 0.0 |
| | (Human), 731 aa (fragment). | 12 . . . 730 | 719/719 | (100%) | |
| AAL67834 | BROMODOMAIN- | 1 . . . 719 | 694/720 | (96%) | 0.0 |
| | CONTAINING PROTEIN BRD4 | 1 . . . 720 | 700/720 | (96%) | |
| | SHORT VARIANT - Mus | | | | |
| | musculus (Mouse), 723 aa. | | | | |

PFam analysis predicts that the NOV79a protein contains the domains shown in the Table 79E.

TABLE 79E
Domain Analysis of NOV79a

| | | Identities/ | |
|---|---|---|---|
| | | Similarities | |
| | NOV79a Match | for the Matched | Expect |
| Pfam Domain | Region | Region | Value |
| bromodomain: | 63 . . . 152 | 42/92 (46%) | 8.6e−45 |
| domain 1 of 2 | | 82/92 (89%) | |
| bromodomain: | 356 . . . 445 | 40/92 (43%) | 3e−40 |
| domain 2 of 2 | | 81/92 (88%) | |

### Example 80

The NOV80 clone was analyzed, and the nucleotide and predicted polypeptide sequences are shown in Table 80A.

TABLE 80A
NOV80 Sequence Analysis

| | |
|---|---|
| SEQ ID NO: 225 | 1776 bp |

| | |
|---|---|
| NOV80a, | TGGTTCGTTTATTCCTGGGGTTGTCATATCATGGCTTATAATGACACAGACAGAAACC |
| CG59572-01 DNA Sequence | |
| | AGACTGAGAAGCTCCTAAAAAGAGTACGAGAACTGGAGCAAGAGGTGCAAAGACTTAA |
| | AAAGGAACAGGCCAAAAATAAGGAGGACTCAAACATTAGAGAAAATTCAGCAGGAGCT |
| | GGAAAAACTAAGCGTGCATTTGATTTCAGTGCTCATGGCCGAAGACACGTAGCCCTAA |
| | GAATAGCCTATATGGGCTGGGGATACCAGGGCTTTGCTAGTCAGGAAAACACAAATAA |
| | TACCATTGAAGAGAAACTGTTTGAAGCTCTAACCAAGACTCGACTAGTAGAAAGCAGA |
| | CAGACATCCAACTATCACCGATGTGGGAGAACAGATAAAGGAGTTAGTGCCTTTGGAC |
| | AGGTGATCTCACTTGACCTTCGCTCTCAGTTTCCAAGGGGCAGGGATTCCGAGGACTT |
| | TAATGTAAAAGAGGAGGCTAATGCTGCTGCTGAAGAGATCCGTTATACCCACATTCTC |
| | AATCGGGTACTCCCTCCAGACATCCGTATATTGGCCTGGGCCCCTGTAGAACCAAGCT |
| | TCAGTGCTAGGTTCAGCTGCCTTGAGCGGACTTACCGCTATTTTTTCCCTCGTGCTGA |
| | TTTAGATATTGTAACCATGGATTATGCAGCTCAGAAGTATGTTGGCACCCATGATTTC |
| | AGGAACTTGTGTAAAATGGATGTAGCCAACGGTGTGATTAATTTTCAGAGGACTATTC |
| | TATCTGCTCAAGTACAGCTAGTGGGCCAGAGCCCAGGTGAGGGGAGATGGCAAGAACC |
| | TTTCCAGTTATGTCAGTTTGAAGTGACTGGCCAGGCATTCCTTTATCATCAAGTCCGA |
| | TGTATGATGGCTATCCTCTTTCTGATTGGCCAAGGAATGGAGAAGCCAGAGATTATTG |
| | ATGAGCTGCTGAATATAGAGAAAAATCCCCAAAAGCCTCAATATAGTATGGCTGTAGA |
| | ATTTCCTCTAGTCTTATATGACTGTAAGTTTGAAAATGTCAAGTGGATCTATGACCAG |
| | GAGGCTCAGGAGTTCAATATTACCCACCTACAACAACTGTGGGCTAATCATGCTGTCA |
| | AAACTCACATGTTGTATAGTATGCTACAAGGACTGGACACTGTTCCAGTACCCTGTGG |
| | AATAGGACCAAAGATGGATGGAATGACAGAATGGGGAAATGTTAAGCCCTCTGTCATA |
| | AAGCAGACCAGTGCCTTTGTAGAAGGAGTGAAGATGCGCACATATAAGCCCCTCATGG |
| | ACCGTCCTAAATGCCAAGGACTGGAATCCCGGATCCAGCATTTTGTACGTAGGGGACG |
| | AATTGAGCACCCACATTTATTCCATGAGGAAGAAACAAAAGCCAAAAGGGACTGTAAT |
| | GACACACTAGAGGAAGAGAATACTAATTTGGAGACACCAACGAAGAGGGTCTGTGTTG |
| | ACACAGAAATTAAAAGCATCATTTAACCATAGACAATTTGCCAGGATCTAGGAACCAC |
| | CTAATGGTAGGTGGACAGAAAAGGAAAAAAAAAAAAATTTACTTGCAAGTACTAGGAA |
| | TTCAGATGATCAGCTCTTAAAAAAAAAAAAAAAGCAAAAAGACTAAAGCCCTATTAAG |
| | GAAGTTATTGCTTTAATAAGAAATTTCAAATATTCTCTTATCCCGGTCCAAAAGGATT |
| | AAGCGATTAAAGAACGTAAAATGGAGATGTATTTACATACACCTGGAAACCTGTGCCT |
| | TGTATTCAAATTCATTAAAGCCTAATCCTGCAAGAA |

| | |
|---|---|
| ORF Start: ATG at 31 | ORF Stop: TAA at 1474 |

| | | |
|---|---|---|
| SEQ ID NO:226 | 481 aa | MW at 55646.8kD |

| | |
|---|---|
| NOV80a, | MAYNDTDRNQTEKLLKRVRELEQEVQRLKKEQAKNKEDSNIRENSAGAGKTKRAFDFS |
| CG59572-01 Protein Sequence | |
| | AHGRRHVALRIAYMGWGYQGFASQENTNNTIEEKLFEALTKTRLVESRQTSNYHRCGR |
| | TDKGVSAFGQVISLDLRSQFPRGRDSEDFNVKEEANAAAEEIRYTHILNRVLPPDIRI |
| | LAWAPVEPSFSARFSCLERTYRYFFPRADLDIVTMDYAAQKYVGTHDFRNLCKMDVAN |
| | GVINFQRTILSAQVQLVGQSPGEGRWQEPFQLCQFEVTGQAFLYHQVRCMMAILFLIG |
| | QGMEKPEIIDELLNIEKNPQKPQYSMAVEFPLVLYDCKFENVKWIYDQEAQEFNITHL |
| | QQLWANHAVKTHMLYSMLQGLDTVPVPCGIGPKMDGMTEWGNVKPSVIKQTSAFVEGV |
| | KMRTYKPLMDRPKCQGLESRIQHFVRRGRIEHPHLFHEEETKAKRDCNDTLEEENTNL |
| | ETPTKRVCVDTEIKSII |

| | |
|---|---|
| SEQ ID NO:227 | 1508 bp |

| | |
|---|---|
| NOV80b, | CATGGCTTATAATGACACAGACAGAAACCAGACTGAGAAGCTCCTAAAAAGAGTACGA |
| CG59572-02 DNA Sequence | |
| | GAACTGGAGCAAGAGGTGCAAAGACTTAAAAAGGAACAGGCCAAAAATAAGGAGGACT |
| | CAAACATTAGAGAAAATTCAGCAGGAGCTGGAAAAACTAAGCGTGCATTTGATTTCAG |
| | TGCTCATGGCCGAAGACACGTAGCCCTAAGAATAGCCTATATGGGCTGGGGATACCAG |
| | GGCTTTGCTAGTCAGGAAAACACAAATAATACCATTGAAGAGAAACTGTTTGAAGCTC |
| | TAACCAAGACTCGACTAGTAGAAAGCAGACAGACATCCAACTATCACCGATGTGGGAG |
| | AACAGATAAAGGAGTTAGTGCCTTTGGACAGGTGATCTCACTTGACCTTCGCTCTCAG |
| | TTTCCAAGGGGCAGGGATTCCGAGGACTTTAATGTAAAAGAGGAGGCTAATGCTGCTG |
| | CTGAAGAGATCCGTTATACCCACATTCTCAATCGGGTACTCCCTCCAGACATCCGTAT |
| | ATTGGCCTGGGCCCCTGTAGAACCAAGCTTCAGTGCTAGGTTCAGCTGCCTTGAGCGG |
| | ACTTACCGCTATTTTTTCCCTCGTGCTGATTTAGATATTGTAACCATGGATTATGCAG |
| | CTCAGAAGTATGTTGGCACCCATGATTTCAGGAACTTGTGTAAAATGGATGTAGCCAA |
| | CGGTGTGATTAATTTTCAGAGGACTATTCTATCTGCTCAAGTACAGCTAGTGGGCCAG |
| | AGCCCAGGTGAGGGGAGATGGCAAGAACCTTTCCAGTTATGTCAGTTTGAAGTGACTG |
| | GCCAGGCATTCCTTTATCATCAAGTCCGATGTATGATGGCTATCCTCTTTCTGATTGG |
| | CCAAGGAATGGAGAAGCCAGAGATTATTGATGAGCTGCTGAATATAGAGAAAAATCCC |
| | CAAAAGCCTCAATATAGTATGGCTGTAGAATTTCCTCTAGTCTTATATGACTGTAAGT |
| | TTGAAAATGTCAAGTGGATCTATGACCAGGAGGCTCAGGAGTTCAATATTACCCACCT |
| | ACAACAACTGTGGGCTAATCATGCTGTCAAAACTCACATGTTGTATAGTATGCTACAA |
| | GGACTGGACACTGTTCCAGTACCCTGTGGAATAGGACCAAAGATGGATGGAATGACAG |
| | AATGGGGAAATGTTAAGCCCTCTGTCATAAAGCAGACCAGTGCCTTTGTAGAAGGAGT |
| | GAAGATGCGCACATATAAGCCCCTCATGGACCGTCCTAAATGCCAAGGACTGGAATCC |
| | CGGATCCAGCATTTTGTACGTAGGGGACGAATTGAGCACCCACATTTATTCCATGAGG |
| | AAGAAACAAAAGCCAAAAGGGACTGTAATGACACACTAGAGGAAGAGAATACTAATTT |
| | GGAGACACCAACGAAGAGGGTCTGTGTTGACACAGAAATTAAAAGTATCATTTAACCA |
| | TAGACAATTTGCCAGGATCTAGGAACCACCTAATGGTAGGTGGACAGAAAAGGAAAAA |

| | |
|---|---|
| ORF Start: ATG at 2 | ORF Stop: TAA at 1445 |

| | | |
|---|---|---|
| SEQ ID NO: 228 | 481 aa | MW at 55646.8 kD |

| | |
|---|---|
| NOV80b, | MAYNDTDRNQTEKLLKRVRELEQEVQRLKKEQAKNKEDSNIRENSAGAGKTKRAFDFS |
| CG59572-02 Protein Sequence | |
| | AHGRRHVALRIAYMGWGYQGFASQENTNNTIEEKLFEALTKTRLVESRQTSNYHRCGR |
| | TDKGVSAFGQVISLDLRSQFPRGRDSEDFNVKEEANAAAEEIRYTHILNRVLPPDIRI |
| | LAWAPVEPSFSARFSCLERTYRYFFPRADLDIVTMDYAAQKYVGTHDFRNLCKMDVAN |
| | GVINFQRTILSAQVQLVGQSPGEGRWQEPFQLCQFEVTGQAFLYHQVRCMMAILFLIG |
| | QGMEKPEIIDELLNIEKNPQKPQYSMAVEFPLVLYDCKFENVKWIYDQEAQEFNITHL |
| | QQLWANHAVKTHMLYSMLQGLDTVPVPCGIGPKMDGMTEWGNVKPSVIKQTSAFVEGV |
| | KMRTYKPLMDRPKCQGLESRIQHFVRRGRIEHPHLFHEEETKAKRDCNDTLEEENTNL |
| | ETPTKRVCVDTEIKSII |

Sequence comparison of the above protein sequences yields the following sequence relationships shown in Table 80B.

TABLE 80B
Comparison of NOV80a against NOV80b.

| | | Identities/ |
|---|---|---|
| | | Similarities |
| Protein | NOV80a Residues/ | for the |
| Sequence | Match Residues | Matched Region |
| NOV80b | 1 . . . 481 | 459/481 (95%) |
| | 1 . . . 481 | 459/481 (95%) |

Further analysis of the NOV80a protein yielded the following properties shown in Table 80C.

TABLE 80C
Protein Sequence Properties NOV80a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0142 probability located |
| | in microbody (peroxisome) |
| SignalP | No Known Signal Sequence Predicted |
| analysis: | |

A search of the NOV80a protein against the Geneseq database, a proprietary database that contains sequences published in patents and patent publication, yielded several homologous proteins shown in Table 80D.

TABLE 80D
Geneseq Results for NOV80a

| | | NOV80a | Identieies/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAM79457 | Human protein SEQ ID NO 3103 - | 1 . . . 481 | 478/481 | (99%) | 0.0 |
| | Homo sapiens, 490 aa. | 10 . . . 490 | 480/481 | (99%) | |
| | [WO200157190-A2, 9 AUG | | | | |
| | 2001] | | | | |
| AAM78473 | Human protein SEQ ID NO 1135 - | 1 . . . 481 | 478/481 | (99%) | 0.0 |
| | Homo sapiens, 481 aa. | 1 . . . 481 | 480/481 | (99%) | |
| | [WO200157190-A2, 9 AUG | | | | |
| | 2001] | | | | |
| AAG64907 | Human depressed growth rate | 209 . . . 431 | 223/223 | (100%) | e−132 |
| | protein DEG1 - Homo sapiens, | 1 . . . 223 | 223/223 | (100%) | |
| | 248 aa. [CN1296014-A, 23 MAY | | | | |
| | 2001] | | | | |
| AAG02637 | Human secreted protein, SEQ ID | 361 . . . 456 | 96/96 | (100%) | 5e−53 |
| | NO: 6718 - Homo sapiens, 96 aa. | 1 . . . 96 | 96/96 | (100%) | |
| | [EP1033401-A2, 6 SEP 2000] | | | | |
| AAB96592 | Putative P. abyssi pseudourydilate | 65 . . . 367 | 79/305 | (25%) | 4e−16 |
| | synthase I - Pyrococcus abyssi, | 3 . . . 261 | 140/305 | (45%) | |
| | 263 aa. [FR2792651-A1, 27 OCT | | | | |
| | 2000] | | | | |

In a BLAST search of public sequence databases, the NOV80a protein was found to have homology to the proteins shown in the BLASTP data in Table 80E.

TABLE 80E
Public BLASTP Results for NOV80a

| | | NOV80a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q9BZE2 | FKSG32 - Homo sapiens (Human), | 1 . . . 481 | 481/481 | (100%) | 0.0 |
| | 481 aa. | 1 . . . 481 | 481/481 | (100%) | |
| Q96J23 | HYPOTHETICAL 55.6 KDA | 1 . . . 481 | 478/481 | (99%) | 0.0 |
| | PROTEIN - Homo sapiens (Human), | 1 . . . 481 | 480/481 | (99%) | |
| | 481 aa. | | | | |
| Q96NB4 | CDNA FLJ31140 FIS, CLONE | 1 . . . 481 | 478/481 | (99%) | 0.0 |
| | IMR322001218, HIGHLY | 1 . . . 481 | 479/481 | (99%) | |
| | SIMILAR TO MUS MUSCULUS | | | | |
| | PSEUDOURIDINE SYNTHASE 3 | | | | |
| | (PUS3) MRNA - Homo sapiens | | | | |
| | (Human), 481 aa. | | | | |
| Q9JI38 | PSEUDOURIDINE SYNTHASE 3 - | 5 . . . 480 | 407/479 | (84%) | 0.0 |
| | Mus musculus (Mouse), 481 aa. | 4 . . . 480 | 434/479 | (89%) | |
| Q9D0F7 | 2610020J05RIK PROTEIN - Mus | 5 . . . 314 | 276/312 | (88%) | e−158 |
| | musculus (Mouse), 316 aa. | 4 . . . 315 | 291/312 | (92%) | |

PFam analysis predicts that the NOV80a protein contains the domains shown in the Table 80F.

TABLE 80F
Domain Analysis of NOV80a

| | | Identities/ | | |
|---|---|---|---|---|
| | | Similarities | | |
| | NOV80a Match | for the Matched | | Expect |
| Pfam Domain | Region | Region | | Value |
| PseudoU_synth_1: | 88 . . . 307 | 70/249 | (28%) | 4.7e−57 |
| domain 1 of 1 | | 176/249 | (71%) | |

### Example 81

The NOV81 clone was analyzed, and the nucleotide and predicted polypeptide sequences are shown in Table 81A.

TABLE 81A
NOV81 Sequence Analysis

| | |
|---|---|
| SEQ ID NO: 229 | 3080 bp |

| | |
|---|---|
| NOV81a, | TTCCAGCCGGCAGGATGGAGGACGAGGAAGGCCCTGAGTATGGCAAACCTGACTTTGT |
| CG59522-01 DNA Sequence | |
| | GCTTTTGGACCAAGTGACCATGGAGGACTTCATGAGGAACCTGCAGCTCAGGTTCGAG |
| | AAGGGCCGCATCTACACCTACATCGGTGAGGTGCTGGTGTCCGTGAACCCCTACCAGG |
| | AGCTGCCCCTGTATGGGCCTGAGGCCATCGCCAGGTACCAGGGCCGTGAGCTCTATGA |
| | GCGGCCACCCCATCTCTATGCTGTGGCCAACGCCGCCTACAAGGCAATGAAGCACCGG |
| | TCCAGGGACACCTGCATCGTCATCTCAGGGGAGAGTGGGGCAGGGAAGACAGAAGCCA |
| | GTAAGCACATCATGCAGTACATCGCTGCTGTCACCAATCCAAGCCAGAGGGCTGAGGT |
| | GGAGAGGGTCAAGGACGTGCTGCTCAAGTCCACCTGTGTGCTGGAGGCCTTTGGCAAT |
| | GCCCGCACCAACCGCAATCACAACTCCAGCCGCTTTGGCAAGTACATGGACATCAACT |
| | TTGACTTCAAGGGGGACCCGATCGGAGGACACATCCACAGCTACCTACTGGAGAAGTC |
| | TCGGGTCCTCAAGCAGCACGTGGGTGAAAGAAACTTCCACGCCTTCTACCAATTGCTG |
| | AGAGGCAGTGAGGACAAGCAGCTGCATGAACTGCACTTGGAGAGAAACCCTGCTGTAT |
| | ACAATTTCACACACCAGGGAGCAGGACTCAACATGACTGTGAGTGATGAGCAGAGCCA |
| | CCAGGCAGTGACCGAGGCCATGAGGGTCATCGGCTTCAGTCCTGAAGAGGTGGAGTCT |
| | GTGCATCGCATCCTGGCTGCCATATTGCACCTGGGAAACATCGAGTTTGTGGAGACGG |
| | AGGAGGGTGGGCTGCAGAAGGAGGGCCTGGCAGTGGCCGAGGAGGCACTGGTGGACCA |
| | TGTGGCTGAGCTGACGGCCACACCCCGGGACCTCGTGCTCCGCTCCCTGCTGGCTCGC |
| | ACAGTTGCCTCGGGAGGCAGGGAACTCATAGAGAAGGGCCACACTGCAGCTGAGGCCA |
| | GCTATGCCCGGGATGCCTGTGCCAAGGCAGTGTACCAGCGGCTGTTTGAGTGGGTGGT |
| | GAACAGGATCAACAGTGTCATGGAACCCCGGGGCCGGGATCCTCGGCGTGATGGCAAG |
| | GACACAGTCATTGGCGTGCTGGACATCTATGGCTTCGAGGTGTTTCCCGTCAACAGTT |
| | TCGAGCAGTTCTGCATCAACTACTGCAACGAGAAGCTGCAGCAGCTATTCATCCAGCT |
| | CATCCTGAAGCAGGAACAGGAAGAGTACGAGCGCGAGGGCATCACCTGGCAGAGCGTT |
| | GAGTATTTCAACAACGCCACCATTGTGGATCTGGTGGAGCGGCCCCACCGTGGCATCC |
| | TGGCCGTGCTGGACGAGGCCTGCAGCTCTGCTGGCACCATCACTGACCGAATCTTCCT |
| | GCAGACCCTGGACATGCACCACCGCCATCACCTACACTACACCAGCCGCCAGCTCTGC |
| | CCCACAGACAAGACCATGGAGTTTGGCCGAGACTTCCGGATCAAGCACTATGCAGGGG |
| | ACGTCACGTACTCCGTGGAAGGCTTCATCGACAAGAACAGAGATTTCCTCTTCCAGGA |
| | CTTCAAGCGGCTGCTGTACAACAGCACGGACCCCACTCTACGGGCCATGTGGCCGGAC |
| | GGGCAGCAGGACATCACAGAGGTGACCAAGCGCCCCCTGACGGCTGGCACACTCTTCA |
| | AGAACTCCATGGTGGCCCTGGTGGAGAACCTTGCCTCCAAGGAGCCCTTCTACGTCCG |
| | CTGCATCAAGCCCAATGAGGACAAGGTAGCTGGGAAGCTGGATGAGAACCACTGTCGC |
| | CACCAGGTCGCATACCTGGGGCTGCTGGAGAATGTGAGGGTCCGCAGGGCTGGCTTCG |
| | CTTCCCGCCAGCCCTACTCTCGATTCCTGCTCAGGTACAAGATGACCTGTGAATACAC |
| | ATGGCCCAACCACCTGCTGGGCTCCGACAAGGCAGCCGTGAGCGCTCTCCTGGAGCAG |
| | CACGGGCTGCAGGGGGACGTGGCCTTTGGCCACAGCAAGCTGTTCATCCGCTCACCCC |
| | GGACACTGGTCACACTGGAGCAGAGCCGAGCCCGCCTCATCCCCATCATTGTGCTGCT |
| | ATTGCAGAAGGCATGGCGGGGCACCTTGGCGAGGTGGCGCTGCCGGAGGCTGAGGGCT |
| | ATCTACACCATCATGCGCTGGTTCCGGAGACACAAGGTGCGGGCTCACCTGGCTGAGC |
| | TGCAGCGGCGATTCCAGGCTGCAAGGCAGCCGCCACTCTACGGGCGTGACCTTGTGTG |
| | GCCGCTGCCCCCTGCTGTGCTGCAGCCCTTCCAGGACACCTGCCACGCACTCTTCTGC |
| | AGGTGGCGGGCCCGGCAGCTGGTGAAGAACATCCCCCCTTCAGACATGCCCCAGATCA |
| | AGGCCAAGGTGGCCGCCATGGGGGCCCTGCAAGGGCTTCGTCAGGACTGGGGCTGCCG |
| | ACGGGCCTGGGCCCGAGACTACCTGTCCTCTGCCACTGACAATCCCACAGCATCAAGC |
| | CTGTTTGCTCAGCGACTAAAGACACTTCAGGACAAAGATGGCTTCGGGGCTGTGCTCT |
| | TTTCAAGCCATGTCCGCAAGGTGAACCGCTTCCACAAGATCCGGAACCGGGCCCTCCT |
| | GCTCACAGACCAGCACCTCTACAAGCTGGACCCTGACCGGCAGTACCGGGTGATGCGG |
| | GCCGTGCCCCTTGAGGCGGTGACGGGGCTGAGCGTGACCAGCGGAGGAGACCAGCTGG |
| | TGGTGCTGCACGCCCGCGGCCAGGACGACCTCGTGGTGTGCCTGCACCGCTCCCGGCC |
| | GCCATTGGACAACCGCGTTGGGGAGCTGGTGGGCGTGCTGGCCGCACACTGCCGCAGG |
| | GAGGGCCGCACCCTGGAGGTTCGCGTCTCCGACTGCATCCCACTAAGCCATCGCGGGG |
| | TCCGGCGCCTCATCTCCGTGGAGCCCAGGCCGGAGCAGCCAGAGCCCGATTTCCGCTG |
| | CGCTCGCGGCTCCTTCACCCTGCTCTGGCCCAGCCGCTGAGCGCCCGCACCCGCCGCA |
| | CCCCGA |

| | |
|---|---|
| ORF Start: ATG at 15 | ORF Stop: TGA at 3054 |

| | | |
|---|---|---|
| SEQ ID NO: 230 | 1013 aa | MW at 116044.5kD |

| | |
|---|---|
| NOV81a, | MEDEEGPEYGKPDFVLLDQVTMEDFMRNLQLRFEKGRIYTYIGEVLVSVNPYQELPLY |
| CG59522-01 Protein | |
| Sequence | GPEAIARYQGRELYERPPHLYAVANAAYKAMKHRSRDTCIVISGESGAGKTEASKHIM |
| | QYIAAVTNPSQRAEVERVKDVLLKSTCVLEAFGNARTNRNHNSSRFGKYMDINFDFKG |
| | DPIGGHIHSYLLEKSRVLKQHVGERNFHAFYQLLRGSEDKQLHELHLERNPAVYNFTH |
| | QGAGLNMTVSDEQSHQAVTEAMRVIGFSPEEVESVHRILAAILHLGNIEFVETEEGGL |
| | QKEGLAVAEEALVDHVAELTATPRDLVLRSLLARTVASGGRELIEKGHTAAEASYARD |
| | ACAKAVYQRLFEWVVNRINSVMEPRGRDPRRDGKDTVIGVLDIYGFEVFPVNSFEQFC |
| | INYCNEKLQQLFIQLILKQEQEEYEREGITWQSVEYFNNATIVDLVERPHRGILAVLD |
| | EACSSAGTITDRIFLQTLDMHHRHHLHYTSRQLCPTDKTMEFGRDFRIKHYAGDVTYS |
| | VEGFIDKNRDFLFQDFKRLLYNSTDPTLRAMWPDGQQDITEVTKRPLTAGTLFKNSMV |
| | ALVENLASKEPFYVRCIKPNEDKVAGKLDENHCRHQVAYLGLLENVRVRRAGFASRQP |
| | YSRFLLRYKMTCEYTWPNHLLGSDKAAVSALLEQHGLQGDVAFGHSKLFIRSPRTLVT |
| | LEQSRARLIPIIVLLLQKAWRGTLARWRCRRLRAIYTIMRWFRRHKVRAHLAELQRRF |
| | QAARQPPLYGRDLVWPLPPAVLQPFQDTCHALFCRWRARQLVKNIPPSDMPQIKAKVA |
| | AMGALQGLRQDWGCRRAWARDYLSSATDNPTASSLFAQRLKTLQDKDGFGAVLFSSHV |
| | RKVNRFHKIRNRALLLTDQHLYKLDPDRQYRVMRAVPLEAVTGLSVTSGGDQLVVLHA |
| | RGQDDLVVCLHRSRPPLDNRVGELVGVLAAHCRREGRTLEVRVSDCIPLSHRGVRRLI |
| | SVEPRPEQPEPDFRCARGSFTLLWPSR |

Further analysis of the NOV81a protein yielded the following properties shown in Table 81B.

TABLE 81B
Protein Sequence Properties NOV81a

| | |
|---|---|
| PSort | 0.8800 probability located in nucleus; 0.3902 probability |
| analysis: | located in microbody (peroxisome); 0.2210 probability |
| | located in lysosome (lumen); 0.1000 probability located |
| | in mitochondrial matrix space |
| SignalP | No Known Signal Sequence Predicted |
| analysis: | |

A search of the NOV81a protein against the Geneseq database, a proprietary database that contains sequences published in patents and patent publication, yielded several homologous proteins shown in Table 81C.

TABLE 81C
Geneseq Results for NOV81a

| | | NOV81a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAU23125 | Novel human enzyme | 1 . . . 1013 | 1009/1018 | (99%) | 0.0 |
| | polypeptide #211-Homo | 9 . . . 1026 | 1011/1018 | (99%) | |
| | sapiens, 1026 aa. | | | | |
| | [WO200155301-A2, 2 AUG | | | | |
| | 2001] | | | | |
| AAU23128 | Novel human enzyme | 1 . . . 853 | 851/858 | (99%) | 0.0 |
| | polypeptide #214 - Homo | 9 . . . 866 | 851/858 | (99%) | |
| | sapiens, 909 aa. [WO200155301- | | | | |
| | A2, 2 AUG 2001] | | | | |
| AAM80123 | Human protein SEQ ID NO 3769 - | 243 . . . 1011 | 438/769 | (56%) | 0.0 |
| | Homo sapiens, 764 aa. | 1 . . . 762 | 570/769 | (73%) | |
| | [WO200157190-A2, 9 AUG | | | | |
| | 2001] | | | | |
| AAM79139 | Human protein SEQ ID NO 1801 - | 254 . . . 1011 | 434/758 | (57%) | 0.0 |
| | Homo sapiens, 753 aa. | 1 . . . 751 | 564/758 | (74%) | |
| | [WO200157190-A2, 9 AUG | | | | |
| | 2001] | | | | |
| AAM39991 | Human polypeptide SEQ ID NO | 10 . . . 933 | 410/966 | (42%) | 0.0 |
| | 3136 - Homo sapiens, 1063 aa. | 47 . . . 986 | 556/966 | (57%) | |
| | [WO200153312-A1, 26 JUL | | | | |
| | 2001] | | | | |

In a BLAST search of public sequence databases, the NOV81a protein was found to have homology to the proteins shown in the BLASTP data in Table 81D.

TABLE 81D
Public BLASTP Results for NOV81a

| | | NOV81a | Identities/ | | |
|---|---|---|---|---|---|
| Protein | | Residues/ | Similarities for | | |
| Accession | | Match | the Matched | | Expect |
| Number | Protein/Organism/Length | Residues | Portion | | Value |
| Q63357 | MYOSIN I - Rattus norvegicus | 1 . . . 1011 | 606/1011 | (59%) | 0.0 |
| | (Rat), 1006 aa. | 1 . . . 1004 | 780/1011 | (76%) | |
| A53933 | myosin I myr 4 - rat, 1006 aa. | 1 . . . 1011 | 604/1011 | (59%) | 0.0 |
| | | 1 . . . 1004 | 778/1011 | (76%) | |
| Q96RI6 | UNCONVENTIONAL MYOSIN | 33 . . . 646 | 612/619 | (98%) | 0.0 |
| | 1G VALINE FORM - Homo | 1 . . . 619 | 612/619 | (98%) | |
| | sapiens, (Human), 633 aa | | | | |
| | (fragment). | | | | |
| Q96RI5 | UNCONVENTIONAL MYOSIN | 33 . . . 646 | 611/619 | (98%) | 0.0 |
| | 1G METHONINE FORM - Homo | 1 . . . 619 | 612/619 | (98%) | |
| | sapiens, (Human), 633 aa | | | | |
| | (fragment). | | | | |
| Q23978 | Myosin IA (MIA) (Brush border | 8 . . . 1012 | 503/1017 | (49%) | 0.0 |
| | myosin IA) (BBMIA) - Drosophila | 6 . . . 1007 | 686/1017 | (66%) | |
| | melanogaster (Fruit fly), 1011 aa. | | | | |

PFam analysis predicts that the NOV81a protein contains the domains shown in the Table 81E.

TABLE 81E
Domain Analysis of NOV81a

| | | Identities/ | | |
|---|---|---|---|---|
| | | Similarities | | |
| | NOV81a Match | for the Matched | | Expect |
| Pfam Domain | Region | Region | | Value |
| PRK: domain 1 of 1 | 97 . . . 109 | 8/13 | (62%) | 3.7 |
| | | 10/13 | (77%) | |
| Vir_DNA_binding: | 575 . . . 592 | 5/18 | (28%) | 8.2 |
| domain 1 of 1 | | 14/18 | (78%) | |
| myosin_head: | 11 . . . 689 | 305/747 | (41%) | 8.1e−288 |
| domain 1 of 1 | | 531/747 | (71%) | |

### Example 82

The NOV82 clone was analyzed, and the nucleotide and predicted polypeptide sequences are shown in Table 82A.

TABLE 82A
NOV82 Sequence Analysis

| | |
|---|---|
| SEQ ID NO: 231 | 1066 bp |

| | |
|---|---|
| NOV82a, | GAACGAATGGGAAACCAGAAATCAGATATTTATGCCCAAGCAAAGCAGGATTTCGTTC |
| CG59520-01 DNA Sequence | |
| | AGCACTACTCCCAGATCGTTAGGGTGCTGACTGAGGATGAGATGGGGCACCCAGAGAC |
| | AGGAGATGCTACTGCCCGGCTCAAGGAGGTCCTGGAGTACAATGCCATTGGAGGCAAG |
| | TATCACCGAGGTTTGATGGTGCTAGTAGCGTTCCGGGAGCTGGTGGAGCCGAGGAAAC |
| | TGGATGCTGATAGTCTCCAGTGGGCACCGACTGTGGGCTGGTATGCGCAACTGCTGCA |
| | AGCTTTCTTCCTGGTGGCAGATGACATTATGGATTCATCCCTTACCTGCCAGGGACAG |
| | ATCTCCTGGTATCAGAAGCTGGGCATGGGTTTGGATGCCATCAATGATGCTATCCTTC |
| | TGGAAGCATGTATCTACTGCCTGCTGAAGCTGTATTGCCGGGAGCAGCCCTATTACCT |
| | GAACCTGATGGAGCTCTTCCAGCAGAATTCTTATCAGACTGAGATTGGGCAGACCCTC |
| | GACCTCATCACAACCCCCCAGGGCAATGTGGATCTTCGCAGATGCACCGAAAAAAGGC |
| | ACAAATCTGTTGTCAAGTACAAGACAGCTTTCTACTCCTTCTACCTTCCTGTAGCTGC |
| | AGCCATGTACATGTCAAGAATGGATGACAAGAAGGAGCACACCAGTGCCAAGAAGATC |
| | CTGCTGGAGATTCAAGAGTTCTTTCAGATTCAGGATGATTACCTTGACTTCTCTGGGG |
| | ACCCCAGTGTGACTGGCAGAGTTGGCAATGACTTCCAGGACAACAAATGCAGCTGGCT |
| | GGTGGTTCAGTGTCTGCTACAGGCCACTCCAGAACAGTACCAGATCCTGAAGGAAAAT |
| | TACAGGCAGAAGGAGGCCGAGAAGGTGGCCCGGGTGAAGGCACTATACGAGGAGCTGG |
| | ATCTGCCAGCCGTGTTCTTGCAGTATGAGAAAGACAGTTACAGCCACGTTATGGGTCT |
| | CATCGAACAGTACGCAGAGCCCCTGCCCCCAGCCATCTTTCTGGGGCTTGTGCACAAA |
| | ATCTACAAGTGGAAAAAGTGAC |

| | |
|---|---|
| ORF Start: ATG at 7 | ORF Stop: TGA at 1063 |

| | | |
|---|---|---|
| SEQ ID NO: 232 | 352 aa | MW at 40740.3 kD |

| | |
|---|---|
| NOV82a, | MGNQKSDIYAQAKQDFVQHYSQIVRVLTEDEMGHPETGDATARLKEVLEYNAIGGKYH |
| CG59520-01 Protein Sequence | |
| | RGLMVLVAFRELVEPRKLDADSLQWAPTVGWYAQLLQAFFLVADDIMDSSLTCQGQIS |
| | WYQKLGMGLDAINDAILLEACIYCLLKLYCREQPYYLNLMELFQQNSYQTEIGQTLDL |
| | ITTPQGNVDLRRCTEKRHKSVVKYKTAFYSFYLPVAAAMYMSRMDDKKEHTSAKKILL |
| | EIQEFFQIQDDYLDFSGDPSVTGRVGNDFQDNKCSWLVVQCLLQATPEQYQILKENYR |
| | QKEAEKVARVKALYEELDLPAVFLQYEKDSYSHVMGLIEQYAEPLPPAIFLGLVHKIY |
| | KWKK |

Further analysis of the NOV82a protein yielded the following properties shown in Table 82B.

TABLE 82B
Protein Sequence Properties NOV82a

| | |
|---|---|
| PSort | 0.4066 probability located in microbody (peroxisome); 0.3000 |
| analysis: | probability located in nucleus; 0.1000 probability located in |
| | mitochondrial matrix space; 0.1000 probability located in |
| | lysosome (lumen) |
| SignalP | No Known Signal Sequence Predicted |
| analysis: | |

A search of the NOV82a protein against the Geneseq database, a proprietary database that contains sequences published in patents and patent publication, yielded several homologous proteins shown in Table 82C.

TABLE 82C
Geneseq Results for NOV82a

| | | NOV82a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG29733 | Arabidopsis thaliana protein | 10 . . . 352 | 147/343 (42%) | 7e−75 |
| | fragment SEQ ID NO: 35427- | 2 . . . 342 | 219/343 (62%) | |
| | Arabidopsis thaliana, 342 aa. | | | |
| | [EP1033405-A2, 6 SEP. 2000] | | | |
| AAG29732 | Arabidopsis thaliana protein | 10 . . . 352 | 147/343 (42%) | 7e−75 |
| | fragment SEQ ID NO: 35426- | 9 . . . 349 | 219/343 (62%) | |
| | Arabidopsis thaliana, 349 aa. | | | |
| | [EP1033405-A2, 6 SEP. 2000] | | | |
| AAG29734 | Arabidopsis thaliana protein | 47 . . . 352 | 138/306 (45%) | 4e−73 |
| | fragment SEQ ID NO: 35428- | 1 . . . 305 | 204/306 (66%) | |
| | Arabidopsis thaliana, 305 aa. | | | |
| | [EP1033405-A2, 6 SEP. 2000] | | | |
| AAY43635 | Amino acid sequence of the farnesyl | 12 . . . 352 | 145/346 (41%) | 4e−69 |
| | pyrophosphate synthase enzyme− | 11 . . . 355 | 208/346 (59%) | |
| | Phaffia rhodozyma, 355 aa. | | | |
| | [EP955363-A2, 10 NOV. 1999] | | | |
| AAB48971 | Sunflower seedling farnesyl | 13 . . . 352 | 138/343 (40%) | 3e−64 |
| | pyrophosphate synthase (FPS)- | 6 . . . 341 | 204/343 (59%) | |
| | Helianthus annuus, 341 aa. | | | |
| | [EP1063297-A1, 27 DEC. 2000] | | | |

In a BLAST search of public sequence databases, the NOV82a protein was found to have homology to the proteins shown in the BLASTP data in Table 82D.

TABLE 82D
Public BLASTP Results for NOV82a

| | | NOV82a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96G29 | FARNESYL DIPHOSPHATE | 2 . . . 352 | 291/351 (82%) | e−168 |
| | SYNTHASE (FARNESYL | 69 . . . 419 | 317/351 (89%) | |
| | PYROPHOSPHATE | | | |
| | SYNTHETASE, | | | |
| | DIMETHYLALLYLTRANSTRANSFERASE, | | | |
| | GERANYLTRANSTRANSFERASE)- | | | |
| | Homo sapiens (Human), 419 aa. | | | |
| P14324 | Farnesyl pyrophosphate synthetase | 2 . . . 352 | 291/351 (82%) | e−168 |
| | (FPP synthetase) (FPS) (Farnesyl | 3 . . . 353 | 317/351 (89%) | |
| | diphosphate synthetase) [Includes: | | | |
| | Dimethylallyltransferase (EC | | | |
| | 2.5.1.1); Geranyltranstransferase | | | |
| | (EC 2.5.1.10)]-Homo sapiens | | | |
| | (Human), 353 aa. | | | |
| A35726 | farnesyl-pyrophosphate synthetase- | 2 . . . 352 | 290/351 (82%) | e−168 |
| | human, 353 aa. | 3 . . . 353 | 316/351 (89%) | |
| AAL58886 | FARNESYL DIPHOSPHATE | 2 . . . 352 | 270/351 (76%) | e−157 |
| | SYNTHASE-Bos taurus (Bovine), | 3 . . . 353 | 308/351 (86%) | |
| | 353 aa. | | | |
| Q14329 | FARNESYL PYROPHOSPHATE | 6 . . . 352 | 268/347 (77%) | e−150 |
| | SYNTHETASE LIKE-4 PROTEIN- | 2 . . . 348 | 295/347 (84%) | |
| | Homo sapiens (Human), 348 aa. | | | |

PFam analysis predicts that the NOV82a protein contains the domains shown in the Table 82E.

TABLE 82E
Domain Analysis of NOV82a

| | | Identities/ | |
|---|---|---|---|
| Pfam | NOV82a | Similarities for | Expect |
| Domain | Match Region | the Matched Region | Value |
| polyprenyl_synt: | 43 . . . 315 | 82/285 (29%) | 6.3e−91 |
| domain 1 of 1 | | 237/285 (83%) | |

### Example 83

The NOV83 clone was analyzed, and the nucleotide and predicted polypeptide sequences are shown in Table 83A.

TABLE 83A
NOV83 Sequence Analysis

| | |
|---|---|
| SEQ ID NO: 233 | 411 bp |

| | |
|---|---|
| NOV83a, | TGCCTACCCCGAGACTGCTGCTGTTCGGAGACCTGCAGGTGAATGCCCCATCACCATG |
| CG59758-01 DNA Sequence | |
| | TCTGACCTGGAGGCAAAACCTTCAACTGAGCATTTGGGGGATAAGATAAAAGATGAAG |
| | ATATTAAACTCAGGGTTATTGGACAGGATAGCAGTGAGATTCATTTCAAAGTGAAAAT |
| | GACAACACCTCTCAAGAAACTCAAGAAATCGTACTGTCAGAGACAGGGCGTTCCAGTG |
| | AATTCCCTCAGGTTTCTCTTTGAAGGTCAGAGAATTGCTGATAATCATACTCCAGAAG |
| | AACTGGGAATGGAGGAAGAAGATGTGATTGAGGTTTATCAGGAACAAATCGGAGGTCA |
| | TTCAACAGTTTAGACATTCTTTTTTTTTTTCCTTTTCCCTCAATCCTTTTTTATTTTT |
| | TTAAA |

| | |
|---|---|
| ORF Start: ATG at 56 | ORF Stop: TAG at 359 |

| | | |
|---|---|---|
| SEQ ID NO: 234 | 101 aa | MW at 11526.0 kD |

| | |
|---|---|
| NOV83a, | MSDLEAKPSTEHLGDKIKDEDIKLRVIGQDSSEIHFKVKMTTPLKKLKKSYCQRQGVP |
| CG59758-01 Protein Sequence | |
| | VNSLRFLFEGQRIADNHTPEELGMEEEDVIEVYQEQIGGHSTV |

| | |
|---|---|
| SEQ ID NO: 235 | 658 bp |

| | |
|---|---|
| NOV83b, | CTACCCCGAGACTGCTGCTGTTCGGAGACCTGCAGGTGAATGCCCCATCACCAATGTCT |
| CG59758-02 DNA Sequence | |
| | GACCTGGAGGCAAAACCTTCAACTGAGCATTTGGGGGATAAGATAAAAGATGAAGATA |
| | TTAAACTCAGGGTTATTGGACAGGATAGCAGTGAGATTCATTTCAAAGTGAAAATGAC |
| | AACACCTCTCAAGAAACTCAAGAAATCGTACTGTCAGAGACAGGGCGTTCCAGTGAAT |
| | TCCCTCAGGTTTCTCTTTGAAGGTCAGAGAATTGCTGATAATCATACTCCAGAAGAAC |
| | TGGGAATGGAGGAAGAAGATGTGATTGAGGTTTATCAGGAACAAATCGGAGGTCATTC |
| | AACAGTTTAGACAATCGGAGGTCATTCAACAGTTTAGACAATCGGAGGTCATTCAACA |
| | GTTTAGACAATCGGAGGTCATTCAACAGTTTAGACAATCGGAGGTCATTCAACAGTTT |
| | AGACAATCGGAGGTCATTCAACAGTTTAGACAATCGGAGGTCATTCAACAGTTTAGAC |
| | AATCGGAGGTCATTCAACAGTTTAGACAATCGGAGGTCATTCAACAGTTTAGACAATC |
| | GGAGGTCATTCAACAGTTTAGACAATCGGAGGTCATTCAACAGTTTAGACAATCGGAG |
| | GTCATTCAACAGTTTAGACA |

| | |
|---|---|
| ORF Start: ATG at 53 | ORF Stop: TAG at 356 |

| | | |
|---|---|---|
| SEQ ID NO: 236 | 101 aa | MW at 11526.0 kD |

| | |
|---|---|
| NOV83b, | MSDLEAKPSTEHLGDKIKDEDIKLRVIGQDSSEIHFKVKMTTPLKKLKKSYCQRQGVP |
| CG59758-02 Protein Sequence | |
| | VNSLRFLFEGQRIADNHTPEELGMEEEDVIEVYQEQIGGHSTV |

Sequence comparison of the above protein sequences yields the following sequence relationships shown in Table 83B.

TABLE 83B
Comparison of NOV83a against NOV83b.

| | | Identities/ |
|---|---|---|
| Protein | NOV83a Residues/ | Similarities for |
| Sequence | Match Residues | the Matched Region |
| NOV83b | 1 . . . 101 | 101/101 (100%) |
| | 1 . . . 101 | 101/101 (100%) |

Further analysis of the NOV83a protein yielded the following properties shown in Table 83C.

TABLE 83C
Protein Sequence Properties NOV83a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV83a protein against the Geneseq database, a proprietary database that contains sequences published in patents and patent publication, yielded several homologous proteins shown in Table 83D.

TABLE 83D
Geneseq Results for NOV83a

| | | NOV83a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM79976 | Human protein SEQ ID NO 3622- | 1 . . . 101 | 100/101 (99%) | 1e−52 |
| | Homo sapiens, 125 aa. | 25 . . . 125 | 100/101 (99%) | |
| | [WO200157190-A2, 9 AUG. 2001] | | | |
| AAM78992 | Human protein SEQ ID NO 1654- | 1 . . . 101 | 100/101 (99%) | 1e−52 |
| | Homo sapiens, 101 aa. | 1 . . . 101 | 100/101 (99%) | |
| | [WO200157190-A2, 9 AUG. 2001] | | | |
| AAY49967 | Human sentrin protein sequence- | 1 . . . 101 | 89/101 (88%) | 2e−45 |
| | Homo sapiens, 101 aa. | 1 . . . 101 | 94/101 (92%) | |
| | [U.S. Pat. No. 5985664-A, | | | |
| | 16 NOV. 1999] | | | |
| AAW87984 | Ubiquitin-like domain of the | 1 . . . 101 | 89/101 (88%) | 2e−45 |
| | protein SUMO1-Mammalia, 101 | 1 . . . 101 | 94/101 (92%) | |
| | aa. [WO9857978-A1, 23 DEC. 1998] | | | |
| AAW60079 | Homo sapiens sentrin-1 | 1 . . . 101 | 89/101 (88%) | 2e−45 |
| | polypeptide-Homo sapiens, 101 | 1 . . . 101 | 94/101 (92%) | |
| | aa. [WO9820038-A1, 14 MAY 1998] | | | |

In a BLAST search of public sequence databases, the NOV83a protein was found to have homology to the proteins shown in the BLASTP data in Table 83E.

TABLE 83E
Public BLASTP Results for NOV83a

| | | NOV83a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q93068 | Ubiquitin-like protein SMT3C | 1 . . . 101 | 89/101 (88%) | 6e−45 |
| | precursor (Ubiquitin-homology domain | 1 . . . 101 | 94/101 (92%) | |
| | protein PIC1) (Ubiquitin-like protein | | | |
| | UBL1) (Ubiquitin-related protein | | | |
| | SUMO-1) (GAP modifying protein 1) | | | |
| | (GMP1) (Sentrin)-Homo sapiens | | | |
| | (Human), and, 101 aa. | | | |
| Q9MZD5 | SENTRIN-Cervus nippon (Sika deer), | 1 . . . 101 | 88/101 (87%) | 2e−44 |
| | 101 aa. | 1 . . . 101 | 93/101 (91%) | |
| O57686 | SUMO-1 PROTEIN-Xenopus laevis | 1 . . . 100 | 83/101 (82%) | 2e−39 |
| | (African clawed frog), 102 aa. | 1 . . . 101 | 90/101 (88%) | |
| Q9PT08 | SMALL UBIQUITIN-RELATED | 1 . . . 97 | 72/97 (74%) | 9e−35 |
| | PROTEIN 1-Oncorhynchus mykiss | 1 . . . 97 | 84/97 (86%) | |
| | (Rainbow trout) (Salmo gairdneri), 101 | | | |
| | aa. | | | |
| Q9D466 | 4933411G06RIK PROTEIN-Mus | 1 . . . 97 | 68/97 (70%) | 8e−30 |
| | musculus (Mouse), 117 aa. | 1 . . . 96 | 80/97 (82%) | |

PFam analysis predicts that the NOV83a protein contains the domains shown in the Table 83F.

TABLE 83F
Domain Analysis of NOV83a

| | | Identities/ | |
|---|---|---|---|
| Pfam | NOV83a | Similarities for | Expect |
| Domain | Match Region | the Matched Region | Value |
| ubiquitin: | 20 . . . 95 | 14/83 (17%) | 4.7e−18 |
| domain 1 of 1 | | 66/83 (80%) | |

### Example 84

The NOV84 clone was analyzed, and the nucleotide and predicted polypeptide sequences are shown in Table 84A.

TABLE 84A
NOV84 Sequence Analysis

| | |
|---|---|
| SEQ ID NO: 237 | 912 bp |

| | |
|---|---|
| NOV84a, | ACTCACTAATGGGCTCGAGCGGCTGCCTGTGTTTCAGCGGCTCGGGGAAATCCACCGT |
| CG59586-01 DNA Sequence | |
| | GGGCGCCCTGCTGGCATCTGAGCTGGGATGGAAATTCTATGATGCTGATGATTATCAC |
| | CCGGAGGAAAATCGAAGGAAGATGGGAAAAGGCATACCGCTCAATGACCAGGACCGGA |
| | TTCCATGGCTCTGTAACTTGCATGACATTTTACTAAGAGATGTAGCCTCGGGACAGCG |
| | TGTGGTTCTAGCCTGTTCAGCCCTGAAGAAAACGTACAGAGACATATTAACACAAGGA |
| | AAAGATGGTGTAGCTCTGAAGTGTGAGGAGTCGGGAAAGGAAGCAAAGCAGGCTGAGA |
| | TGCAGCTCCTGGTGGTCCATCTGAGCGGGTCGTTTGAGGTCATCTCTGGACGCTTACT |
| | CAAAAGAGAGGGACATTTTATGCCCCCTGAATTATTGCAGTCCCAGTTTGAGACTCTG |
| | GAGCCCCCAGCAGCTCCAGAAAACTTTATCCAAATAAGTGTGGACAAAAATGTTTCAG |
| | AGATAATTGCTACAATTATGGAAACCCTAAAAATGAAATGACAATGATTTTGTATCAG |
| | TGGTCCAAACAGAACTAAGCATAAATCATTGTGCCATCCCAAACCTCGTTCCAGCCGC |
| | CTTGCCCATACTAGATTCTAAATGTTTCTAAAGGCAAACCCCAATGTGTCAAGACAGA |
| | CTTGTTTAGGTGTAATTTTAGGAATTATGCTGGTTCATCAGGAAGCAGAGGGGGAGTT |
| | TTAAAAGTCAAGCTTAAATTGAAGTTTAAATTCATCTATAACCAAATCAAATGATCAG |
| | AGGAAATTCTGTAATCAATGCTGGAAATCGTTACATTGTTTAGAACATTCTTGCTCAT |
| | GCCTGTATTTGCACAAATAAATGAAACTTCGCTGTAAAAAAA |

| | |
|---|---|
| ORF Start: ATG at 9 | ORF Stop: TGA at 561 |

| | | |
|---|---|---|
| SEQ ID NO: 238 | 184 aa | MW at 20352.2 kD |

| | |
|---|---|
| NOV84a, | MGSSGCLCFSGSGKSTVGALLASELGWKFYDADDYHPEENRRKMGKGIPLNDQDRIPW |
| CG59586-01 Protein Sequence | |
| | LCNLHDILLRDVASGQRVVLACSALKKTYRDILTQGKDGVALKCEESGKEAKQAEMQL |
| | LVVHLSGSFEVISGRLLKREGHFMPPELLQSQFETLEPPAAPENFIQISVDKNVSEII |
| | ATIMETLKMK |

Further analysis of the NOV84a protein yielded the following properties shown in Table 84B.

TABLE 84B
Protein Sequence Properties NOV84a

| | |
|---|---|
| PSort | 0.6500 probability located in cytoplasm; 0.1000 probability |
| analysis: | located in mitochondrial matrix space; 0.1000 probability |
| | located in lysosome (lumen); 0.1000 probability located in |
| | plasma membrane |
| SignalP | No Known Signal Sequence Predicted |
| analysis: | |

A search of the NOV84a protein against the Geneseq database, a proprietary database that contains sequences published in patents and patent publication, yielded several homologous proteins shown in Table 84C.

TABLE 84C
Geneseq Results for NOV84a

| | | NOV84a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG73989 | Human colon cancer antigen | 10 . . . 184 | 175/175 (100%) | 1e−97 |
| | protein SEQ ID NO: 4753-Homo | 19 . . . 193 | 175/175 (100%) | |
| | sapiens, 193 aa. [WO200122920- | | | |
| | A2, 5 APR. 2001] | | | |
| AAB58998 | Breast and ovarian cancer | 10 . . . 184 | 175/175 (100%) | 1e−97 |
| | associated antigen protein sequence | 19 . . . 193 | 175/175 (100%) | |
| | SEQ ID 706-Homo sapiens, 193 | | | |
| | aa. [WO200055173-A1, | | | |
| | 21 SEP. 2000] | | | |
| AAM89100 | Human immune/haematopoietic | 24 . . . 126 | 70/103 (67%) | 1e−34 |
| | antigen SEQ ID NO: 16693-Homo | 22 . . . 124 | 77/103 (73%) | |
| | sapiens, 133 aa. [WO200157182- | | | |
| | A2, 9 AUG. 2001] | | | |
| AAG50675 | Arabidopsis thaliana protein | 10 . . . 179 | 75/173 (43%) | 4e−28 |
| | fragment SEQ ID NO: 64243- | 4 . . . 167 | 102/173 (58%) | |
| | Arabidopsis thaliana, 175 aa. | | | |
| | [EP1033405-A2, 6 SEP. 2000] | | | |
| AAG50674 | Arabidopsis thaliana protein | 10 . . . 179 | 75/173 (43%) | 4e−28 |
| | fragment SEQ ID NO: 64242- | 16 . . . 179 | 102/173 (58%) | |
| | Arabidopsis thaliana, 187 aa. | | | |
| | [EP1033405-A2, 6 SEP. 2000] | | | |

In a BLAST search of public sequence databases, the NOV84a protein was found to have homology to the proteins shown in the BLASTP data in Table 84D.

TABLE 84D
Public BLASTP Results for NOV84a

| | | NOV84a | | |
|---|---|---|---|---|
| Protein | | Residues/ | Identities/ | |
| Accession | | Match | Similarities for | Expect |
| Number | Protein/Organism/Length | Residues | the Matched Portion | Value |
| BAB74785 | GLUCONOKINASE-Anabaena | 10 . . . 183 | 72/174 (41%) | 1e−30 |
| | sp. (strain PCC 7120), 160 aa. | 9 . . . 160 | 101/174 (57%) | |
| Q9RT56 | THERMORESISTANT | 10 . . . 183 | 66/174 (37%) | 1e−29 |
| | GLUCONOKINASE- | 4 . . . 159 | 101/174 (57%) | |
| | Deinococcus radiodurans, 172 aa. | | | |
| CAC93415 | PUTATIVE GLUCONOKINASE | 10 . . . 174 | 68/166 (40%) | 2e−29 |
| | (EC 2.7.1.12)-Yersinia pestis, | 12 . . . 159 | 95/166 (56%) | |
| | 167 aa. | | | |
| Q9CMM6 | GLK-Pasteurella multocida, 172 | 10 . . . 182 | 68/174 (39%) | 2e−29 |
| | aa. | 15 . . . 169 | 99/174 (56%) | |
| AAK86014 | AGR_C_329P-Agrobacterium | 10 . . . 182 | 74/173 (42%) | 6e−29 |
| | tumefaciens str. C58 (Cereon), | 5 . . . 159 | 98/173 (55%) | |
| | 163 aa. | | | |

PFam analysis predicts that the NOV84a protein contains the domains shown in the Table 84E.

TABLE 84E
Domain Analysis of NOV84a

| | | Identities/ | |
|---|---|---|---|
| Pfam | NOV84a | Similarities for | Expect |
| Domain | Match Region | the Matched Region | Value |
| SKI: domain 1 of 1 | 9 . . . 182 | 37/206 (18%) | 1.1 |
| | | 114/206 (55%) | |

### Example 85

The NOV85 clone was analyzed, and the nucleotide and predicted polypeptide sequences are shown in Table 85A.

TABLE 85A
NOV85 Sequence Analysis

| | |
|---|---|
| SEQ ID NO: 239 | 4332 bp |

| | |
|---|---|
| NOV85a, | GGCGTATTAACGCGCGGGTGCACACCCCCACGGGCGCGCAATGAACAACTATGTGCTT |
| CG59704-01 DNA Sequence | |
| | AATGACGAGATCGGCCAGGGTGCCTTCAGCACTATTTACAAGGGCCGCTATCGCACCA |
| | CCACCGAGTTCTACGCGATTGCTTCCATCGACAAGAAGCGACGGGAGCGCGTCGTGAA |
| | CTGCGTTCAGCTGTTACGCTCCATGCACCACTCAAACGTCATAGAGTTCCACAACTGG |
| | TATGAGACCAACAATCACTTGTGGATCATTACGGAGTACTGCACCGGCGGAGACATGA |
| | GCACGATCCTCCGCTCGAACATTAATCTCACCACTCAGGCGGTCCAGGCGTTCGGCCG |
| | TGATGTGGCGATGGGCCTCATGTACATCCACAGTAAGGGTGTCGTGTATAACGACTTG |
| | CAGACTCGCAATCTGCTGATGGACTCCGCAGCAATGCTGCGCTTCCACGACTTTAGCT |
| | TGGCCTGTCTCTTCCAAGACGCGGCGACGCGGCCACTGGTGGGGACGCCACTGTACAT |
| | GGCCCCCGAGTTGTTCATGGCGGATCGCCCGCTGTACTCGATGGCATCAGACCTGTGG |
| | TCCTTCGGTTGTGTGCTGCACGAGCTGGCGACAGGCAAGCCGCCCTTTGCCGCATCCG |
| | ACCTCGAGACGCTGCTGGGCGACATACTGACGAGTCCGACGCCAGCGGTGCCTGGTGC |
| | GCCGGAGTCCTTTCAAACGCTCCTGTGCGGCCTGCTGGAAAAGGACCCGTTGAAGCGC |
| | TACGCGTGGGTCGATGTTGTCCGCAGCGAGTTCTGGGATGAGCCCTTGCCGCTGCCGA |
| | GCAACGGCTTTCCATCTCAGGTGGCGTGGGAGGACTACAAGCGTTCGCGTTCTGGACG |
| | CGGTGCGAGTCAGTATAATTGGACGGACTCCGATGTGCGTGTGGCAGTGGCTCACGCC |
| | GTGGGGGCAGCGAAATCAAACGCTTCTACGCACAACGTGGAGGAGAGGGAGCGAGCGG |
| | CTGCGACGTTGAACGTCGCGAAGGAGCTGGACTTCACTGCAAGCGCGGCGATGTTGCT |
| | GGAACGGTTACCGGAGCGGACACAGGAGCGTGCTGCGCACGCAACAGGCCATGTCGCG |
| | ACCGCGCACGGCAGCCTGGTGCACGGCTGCCCATCCACGGCCTCAGCGGCGACCTCGC |
| | CAAGACGTTCAAGGACAAGGCGGCGCTGCTCAAGATTGTGGAAGAGGTCAAAACCGCT |
| | GTCGAGGGCTTCAAGCCGTGGGTGTCCTTCCACGTCGCTGCGCCACCCGGGCATGAGG |
| | GAGCGCCACTGGACCGGCTTGTCTCAGAAGCTCGGGATGAAGCTGGTGCCTGGCGACA |
| | CACTGATGCTTCTGGAGGACTGCGAACCGCTGCTAGCGCACCGCGACACCATTATCAG |
| | CTACTGCGAGGTGGCCGCGAAGGAGTCGCAGATCGAGATGACGCTCAAGGACATGCGT |
| | GCCAAGTGGGAGACCAAGTGCTTCATCATCGAGGCATACAAGGAGACAGGCACGTACA |
| | TCCTCAAGGACACCTCCGAGGTGGTGGAGCTCCTCGACGAGCACCTCAACGTCGTCCA |
| | GCAGCTGCAGTTCTCTCCATTCAAGGGCTACTTCGAGGAGTCCATCACGGACTGGGAG |
| | CGCTCCCTCAACCTCATCTCCGACATACTCGAACAATGGCTGGAGTGCCAGCGAGCGT |
| | GGCGTTATCTGGAGCCGATCCTCAACTCGGAGGACATCGCCATGCAGCTACCGCGACT |
| | GTCCACGCTGTTCGAGAAGGTGGACCGCACATGGAGACGTGTCATGGGCAACGCGCAC |
| | GCGCAGCCAAACGCACTCGAGTACTGCATTGGCACAAACAAGCTCTTGGACCACCTGC |
| | GCGAGGCGAACCGGCTCCTCGAAGTGCTGCAGCACTTGATGGCGCAGAAGGTCAACGT |
| | TGCCGCTGTTGGTCCGACTGGCACCGGCAAGTCCATCTCACTCGCGCGTCTCGTGCTT |
| | GGCGGCGGCATGCCGGCCAACTTTCTTGGCCTCAACTTCACCTTCTCGGCGCAGACAA |
| | AGTGCACAGTGTTGCAGAATTCACTGATGGCCAAGTTCGATAAGCGGCGCTCGCACGT |
| | CTACGGCGCCCCTGCCGGTAAGCACTTTCTCATCTTCATTGACGACGCGAACCTGCCG |
| | CAGCCAGAGAAGTACGGCGCGCAGCCCCCGGTGGAGCTTCTGCGGCAGATGCTCGCCC |
| | AAGGCGGCTTCTACAACTTTACAGGTGGCATCAAGTGGTCCTCCATCATCGACTGCTC |
| | GCTTGCGCTGGCGATGGGGCCGCCTGGCGGGGGCCGCAGCCGGGTTTCGAACCGCTTT |
| | ATGCGCTACTTCAATTACCTTGCCTTCCCCGAGATGTCGGACATGTCGAAGCGAACGA |
| | TCTTGCAGGCCATCCTCGTCGGCGGCCTCGCGCAGAGCGGCCTCGCTGACCGCCTCGC |
| | GAACGTCGCCTCCGCCGTGGTCGATAGCACGTTGCGGGTGTTTCGCAAGTGCACCCAG |
| | GTCTTTCTGCCGACCCCGGCGCACGTGCACTACTCCTTCAACATGCGGGATGTGATGC |
| | GTGTTTTTCCCCTCTTGTACACAGCAGACAAGTCGGTGCTGCAGTCGGAGGAATCCAT |
| | CGTGCGGCTGTGGATGCACGAGATGCAGCGCGTCTTCTACGATCGCCTCGTCGACGCG |
| | ACAGACAAGGGTCTGTTCATCGAGTACCTCAATGCCGAGCTGCCGTCCATGGGGGTGG |
| | ACAAGTCCTACAACGAGGTAGTGAAGGCTGACCGCCTCATCTTTGCCGACGTACTGAG |
| | CGACAAGGGCGTGTACGAGCAGATTACCGACATGAACGCCCTCACGACACGCATGAAT |
| | GAGCTGCTGGAGGCGTACAATGACGAGAATGAAGTGAAGATGAACCTCGTGCTCTTCC |
| | TCGACGCCATCGAGCATGTCTGCCGTATCTCGCGCGTGCTGCGACTGCCGAACGGGCA |
| | CTGCCTCCTCCTCGGCGTTGGCGGGTCGGGACGCAAGTCACTCACGCGCCTGGCTTGT |
| | TCTCTGATTGCCGAGATGGAGGTGTTCACGATTGAGCTGTCGAAGAACTTCGGTGTCA |
| | AGGAATGGCACGAGAGCCTCGCGAAGTTGCTGCTCGAGTGTGGCAAGGACGAGAAGAA |
| | GCGGACGTTTCTCTTCGCCGACACCCAGCTGGCGCATCCGACGTTTCTGGAGGATGTG |
| | GCGGGCCTGCTCACATCGGGTGATGTGCCGAACCTCTTTGAGGACCAAGATATCGAGC |
| | TCATCAACGACAAGTTTCGCGGCGTCTGCCTAAGCGAGAACCTGCCAACGACGAAGGT |
| | GTCGGTGTACGCGCGCTTTGTGAAGGAGGCGCGAGCCAACCTGCACCTTGTGCTCGCC |
| | TTCTCTCCCATCGGAGAGGCGTTTCGCAGCCGCCTGCGTATGTTCCCATCGCTCATTG |
| | CGTGCTGCACAATCGACTGGTTTGCTGAGTGGCCATCCGAGGCGCTACTGTCGGTAGC |
| | CGCAGTGCAGCTGAACGCCGGCGACGTTACTGACGTCATGGGGGCGGCAAGCCATGCC |
| | GACTTGCCGGGCTGCTTCCAGGCAGTGCACCGCGCGGCGGCGGAGGTGACGGAGCGCT |
| | TCTTCACGGAAACGCGTCGTCGCTCGTACGTGACGCCGACGTCCTATCTGTCGCTCCT |
| | CTCCAACTTCAAAGTGATGGCGGCGGCAAAACGCCGCTTCGTTCGCGAGCAGCGCGGC |
| | CGCCTCGAGAAGGGGCTGGAGAAGCTGCGGCACACCGAGGTGCAAGTGGCGGAGCTGG |
| | AGGCCCAGCTCAAGGCGCAGCAGCCGGTTCTGGTGCAGAAAAAGGCAGAGATTCAGTC |
| | GATGATGGAGCGGCTGACGGTGGACCGAAAGGAGGCGGCGGTGAAGGAGGCGGACGCG |
| | CGCAGGGAGGCCCAGCTTCCCGGTGGCCGTGCTGCATACGGCGGTGAAGATGACGAAT |
| | GAGCCGCCGATGGGGCTGCGGGCGAACGTGATGCGCTCCTACTACGGCTTCACTCCCG |
| | AGGACCTCGAGCAGGAGGAGAAGCCCGCCGAGTTCAAAAAGATGTTGATGGCATCCGC |
| | ATGCCTGGTCCCATACCCGAGCACTGAAGAGCAGGGTCTCTGGAGCCTGGCATCGTGG |
| | GGTGGCCCTCAGCTTCCCCACTCACTGTGGGAAGTTTCCTTAGTGTCTCTGAGCCTGT |
| | TTCCTCATCCGTTGCCTGAGGATAAACCTGCTTCAGGATTGTTGGTGAAAAGACTTCC |
| | CTCACCTAGCTTCTGTAACGCCACTGCATGCCACCACTGCTGAGTACTGTTTGTTTGC |
| | TAGGTTGGTGTCATTCTCATTTTACCAGAAAGTGAAGCTC |

| | |
|---|---|
| ORF Start: ATG at 41 | ORF Stop: TGA at 3944 |

| | | |
|---|---|---|
| SEQ ID NO: 240 | 1301 aa | MW at 146115.7 kD |

| | |
|---|---|
| NOV85a, | MNNYVLNDEIGQGAFSTIYKGRYRTTTEFYAIASIDKKRRERVVNCVQLLRSMHHSNV |
| CG59704-01 Protein Sequence | |
| | IEFHNWYETNNHLWIITEYCTGGDMSTILRSNINLTTQAVQAFGRDVAMGLMYIHSKG |
| | VVYNDLQTRNLLMDSAAMLRFHDFSLACLFQDAATRPLVGTPLYMAPELFMADRPLYS |
| | MASDLWSFGCVLHELATGKPPFAASDLETLLGDILTSPTPAVPGAPESFQTLLCGLLE |
| | KDPLKRYAWVDVVRSEFWDEPLPLPSNGFPSQVAWEDYKRSRSGRGASQYNWTDSDVR |
| | VAVAHAVGAAKSNASTHNVEERERAAATLNVAKELDFTASAAMLLERLPERTQERAAH |
| | ATGHVATAHGSLVHGCPSTASAATSPRRSRTRRRCSRLWKRSKPLSRASSRGCPSTSL |
| | RHPGMRERHWTGLSQKLGMKLVPGDTLMLLEDCEPLLAHRDTIISYCEVAAKESQIEM |
| | TLKDMRAKWETKCFIIEAYKETGTYILKDTSEVVELLDEHLNVVQQLQFSPFKGYFEE |
| | SITDWERSLNLISDILEQWLECQRAWRYLEPILNSEDIAMQLPRLSTLFEKVDRTWRR |
| | VMGNAHAQPNALEYCIGTNKLLDHLREANRLLEVLQHLMAQKVNVAAVGPTGTGKSIS |
| | LARLVLGGGMPANFLGLNFTFSAQTKCTVLQNSLMAKFDKRRSHVYGAPAGKHFLIFI |
| | DDANLPQPEKYGAQPPVELLRQMLAQGGFYNFTGGIKWSSIIDCSLALAMGPPGGGRS |
| | RVSNRFMRYFNYLAFPEMSDMSKRTILQAILVGGLAQSGLADRLANVASAVVDSTLRV |
| | FRKCTQVFLPTPAHVHYSFNMRDVMRVFPLLYTADKSVLQSEESIVRLWMHEMQRVFY |
| | DRLVDATDKGLFIEYLNAELPSMGVDKSYNEVVKADRLIFADVLSDKGVYEQITDMNA |
| | LTTRMNELLEAYNDENEVKMNLVLFLDAIEHVCRISRVLRLPNGHCLLLGVGGSGRKS |
| | LTRLACSLIAEMEVFTIELSKNFGVKEWHESLAKLLLECGKDEKKRTFLFADTQLAHP |
| | TFLEDVAGLLTSGDVPNLFEDQDIELINDKFRGVCLSENLPTTKVSVYARFVKEARAN |
| | LHLVLAFSPIGEAFRSRLRMFPSLIACCTIDWFAEWPSEALLSVAAVQLNAGDVTDVM |
| | GAASHADLPGCFQAVHRAAAEVTERFFTETRRRSYVTPTSYLSLLSNFKVMAAAKRRF |
| | VREQRGRLEKGLEKLRHTEVQVAELEAQLKAQQPVLVQKKAEIQSMMERLTVDRKEAA |
| | VKEADARREAQLPGGRAAYGGEDDE |

Further analysis of the NOV85a protein yielded the following properties shown in Table 85B.

TABLE 85B
Protein Sequence Properties NOV85a

| | |
|---|---|
| PSort | 0.8800 probability located in nucleus; 0.3562 probability |
| analysis: | located in microbody (peroxisome); 0.1671 probability located |
| | in lysosome (lumen); 0.1000 probability located in |
| | mitochondrial matrix space |
| SignalP | No Known Signal Sequence Predicted |
| analysis: | |

A search of the NOV85a protein against the Geneseq database, a proprietary database that contains sequences published in patents and patent publication, yielded several homologous proteins shown in Table 85C.

TABLE 85C
Geneseq Results for NOV85a

| | | NOV85a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM79863 | Human protein SEQ ID NO 3509- | 602 . . . 1287 | 218/692 (31%) | 1e−89 |
| | Homo sapiens, 2127 aa. | 168 . . . 847 | 347/692 (49%) | |
| | [WO200157190-A2, | | | |
| | 9 AUG. 2001] | | | |
| AAM79862 | Human protein SEQ ID NO 3508- | 602 . . . 1287 | 218/692 (31%) | 1e−89 |
| | Homo sapiens, 2127 aa. | 168 . . . 847 | 347/692 (49%) | |
| | [WO200157190-A2, | | | |
| | 9 AUG. 2001] | | | |
| AAM78879 | Human protein SEQ ID NO 1541- | 602 . . . 1287 | 218/692 (31%) | 1e−89 |
| | Homo sapiens, 2143 aa. | 108 . . . 787 | 347/692 (49%) | |
| | [WO200157190-A2, | | | |
| | 9 AUG. 2001] | | | |
| AAM78878 | Human protein SEQ ID NO 1540- | 602 . . . 1287 | 218/692 (31%) | 1e−89 |
| | Homo sapiens, 2067 aa. | 108 . . . 787 | 347/692 (49%) | |
| | [WO200157190-A2, | | | |
| | 9 AUG. 2001] | | | |
| AAM80293 | Human protein SEQ ID NO 3945- | 910 . . . 1293 | 153/393 (38%) | 5e−70 |
| | Homo sapiens, 1774 aa. | 33 . . . 405 | 227/393 (56%) | |
| | [WO200157190-A2, | | | |
| | 9 AUG. 2001] | | | |

In a BLAST search of public sequence databases, the NOV85a protein was found to have homology to the proteins shown in the BLASTP data in Table 85D.

TABLE 85D
Public BLASTP Results for NOV85a

| | | NOV85A | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAL37427 | CILIARY DYNEIN HEAVY | 628 . . . 1293 | 271/692 (39%) | e−132 |
| | CHAIN 7 - Homo sapiens | 1975 . . . 2655 | 395/692 (56%) | |
| | (Human), 4024 aa. | | | |
| Q27812 | DYNEIN HEAVY CHAIN | 601 . . . 1247 | 264/667 (39%) | e−127 |
| | ISOTYPE 7B (EC 3.6.1.3) - | 654 . . . 1310 | 389/667 (57%) | |
| | Tripneustes gratilla (Hawaian sea | | | |
| | urchin), 1314 aa (fragment). | | | |
| Q9MBF8 | 1 BETA DYNEIN HEAVY | 611 . . . 1293 | 257/693 (37%) | e−117 |
| | CHAIN - Chlamydomonas | 2486 . . . 3159 | 377/693 (54%) | |
| | reinhardtii, 4513 aa. | | | |
| Q9VJC6 | DHC36C PROTEIN - Drosophila | 596 . . . 1275 | 249/699 (35%) | e−116 |
| | melanogaster (Fruit fly), 4010 aa. | 1913 . . . 2604 | 383/699 (54%) | |
| Q9VWZ3 | DHC16F PROTEIN - Drosophila | 618 . . . 1301 | 248/704 (35%) | e−108 |
| | melanogaster (Fruit fly), 4081 aa. | 2022 . . . 2709 | 380/704 (53%) | |

PFam analysis predicts that the NOV85a protein contains the domains shown in the Table 85E.

TABLE 85E
Domain Analysis of NOV85a

| | | Identities/ | | |
|---|---|---|---|---|
| | | Similarities | | |
| | NOV85a Match | for the | | Expect |
| Pfam Domain | Region | Matched Region | | Value |
| pkinase: domain 1 of 1 | 4 . . . 250 | 80/286 | (28%) | 6.8e−62 |
| | | 190/286 | (66%) | |
| DEAD: domain 1 of 1 | 613 . . . 637 | 7/25 | (28%) | 0.83 |
| | | 22/25 | (88%) | |
| dNK: domain 1 of 1 | 865 . . . 1020 | 32/179 | (18%) | 6.8 |
| | | 101/179 | (56%) | |

The NOV86 clone was analyzed, and the nucleotide and predicted polypeptide sequences are shown in Table 86A.

TABLE 86A
NOV86 Sequence Analysis

| | |
|---|---|
| SEQ ID NO: 241 | 1420 bp |

| | |
|---|---|
| NOV86a, | GTCCAGCTTTAGCTCTCTGCTCGCCGCCGCCGCTGTCGCCGCCACCTCCTCTGATCTA |
| CG59628-01 DNA Sequence | |
| | CGAAAGTCATGTTACCCAACACCGGGAGGCTGGCAGGATGTACAGTTTTTTATCACAGG |
| | TGCAAGCCGTGGCATTGGCAAAGCTATTGCATTGAAAGCAGCAAAGGATGGAGCAAAT |
| | ATTGTTATTGCTGCAAAGACCGCCCAGCCACATCCAAAACTTCTAGGCACAATCTATA |
| | CTGCTGCTGAAGAAATTGAAGCAGTTGGAGGAAAGGCCTTGCCATGTATTGTTGATGT |
| | GAGAGATGAACAGCAGATCAGTGCTGCAGTGGAGAAAGCCATCAAGAAATTTGGAGGA |
| | ATTGATATTCTGGTAAATAATGCCAGTGCCATTAGTTTGACCAATACATTGGACACAC |
| | CTACCAAGAGATTGGATCTGATGATGAACGTGAACACCAGAGGCACCTACCTTGCATC |
| | TAAAGCATGTATTCCTTATTTGAAAAAGAGCAAAGTTGCTCATATCCTCAATATCAGT |
| | CCACCACTGAACCTAAATCCAGTTTGGTTCAAACAGCACTGTGCTTATACCATTGCTA |
| | AGTATGGTATGTCTATGTATGTGCTTGGAATGGCAGAAGAATTTAAAGGTGAAATTGC |
| | AGTCAATGCATTATGGCCTAAAACAGCCATACACACTGCTGCTATGGATATGCTGGGA |
| | GGACCTGGTATCGAAAGCCAGTGTAGAAAAGTTGATATCATTGCAGATGCAGCATATT |
| | CCATTTTCCAAAAGCCAAAAAGTTTTACTGGCAACTTTGTCATTGATGAAAATATCTT |
| | AAAAGAAGAAGGAATAGAAAATTTTGACGTTTATGCAATTAAACCAGGTCATCCTTTG |
| | CAACCAGATTTCTTCTTAGATGAATACCCAGAAGCAGTTAGCAAGAAAGTGGAATCAA |
| | CTGGTGCTGTTCCAGAATTCAAAGAAGAGAAACTGCAGCTGCAACCAAAACCACGTTC |
| | TGGAGCTGTGGAAGAAACATTTAGAATTGTTAAGGACTCTCTCAGTGATGATGTTGTT |
| | AAAGCCACTCAAGCAATCTATCTGTTTGAACTCTCCGGTGAAGATGGTGGCACGTGGT |
| | TTCTTGATCTGAAAAGCAAGGGTGGGAATGTCGGATATGGAGAGCCTTCTGATCAGGC |
| | AGATGTGGTGATGAGTATGACTACTGATGACTTTGTAAAAATGTTTTCAGGTAAACTA |
| | AAACCAACAATGGCATTCATGTCAGGGAAATTGAAGATTAAAGGTAACATGGCCCTAG |
| | CAATCAAATTGGAGAAGCTAATGAATCAGATGAATGCCAGACTGTGAAGGAAAATATA |
| | AAAAAAAAGTCGACTGCTATGCTCAAAAAGTAAAAAAAGCTCAACAGTTAAAATCTAA |
| | TGTTTGTTTTCTTTCCTGTTATATTATA |

| | |
|---|---|
| ORF Start: ATG at 67 | ORF Stop: TGA at 1321 |

| | | |
|---|---|---|
| SEQ ID NO: 242 | 418 aa | MW at 45394.2 kD |

| | |
|---|---|
| NOV86a, | MLPNTGRLAGCTVFITGASRGIGKAIALKAAKDGANIVIAAKTAQPHPKLLGTIYTAA |
| CG59628-01 Protein Sequence | |
| | EEIEAVGGKALPCIVDVRDEQQISAAVEKAIKKFGGIDILVNNASAISLTNTLDTPTK |
| | RLDLMMNVNTRGTYLASKACIPYLKKSKVAHILNISPPLNLNPVWFKQHCAYTIAKYG |
| | MSMYVLGMAEEFKGEIAVNALWPKTAIHTAAMDMLGGPGIESQCRKVDIIADAAYSIF |
| | QKPKSFTGNFVIDENILKEEGIENFDVYAIKPGHPLQPDFFLDEYPEAVSKKVESTGA |
| | VPEFKEEKLQLQPKPRSGAVEETFRIVKDSLSDDVVKATQAIYLFELSGEDGGTWFLD |
| | LKSKGGNVGYGEPSDQADVVMSMTTDDFVKMFSGKLKPTMAFMSGKLKIKGNMALAIK |
| | LEKLMNQMNARL |

Further analysis of the NOV86a protein yielded the following properties shown in Table 86B.

TABLE 86B
Protein Sequence Properties NOV86a

| | |
|---|---|
| PSort | 0.5500 probability located in endoplasmic reticulum |
| analysis: | (membrane); 0.5000 probability located in microbody |
| | (peroxisome); 0.1900 probability located in |
| | lysosome (lumen); 0.1000 probability located |
| | in endoplasmic reticulum (lumen) |
| SignalP | No Known Signal Sequence Predicted |
| analysis: | |

A search of the NOV86a protein against the Geneseq database, a proprietary database that contains sequences published in patents and patent publication, yielded several homologous proteins shown in Table 86C.

TABLE 86C
Geneseq Results for NOV86a

| | | NOV86a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAG81260 | Human AFP protein sequence SEQ | 1 . . . 418 | 418/418 | (100%) | 0.0 |
| | ID NO:38 - Homo sapiens, 418 aa. | 1 . . . 418 | 418/418 | (100%) | |
| | [WO200129221-A2, 26 APR | | | | |
| | 2001] | | | | |
| AAB84367 | Amino acid sequence of human | 1 . . . 418 | 418/418 | (100%) | 0.0 |
| | alcohol dehydrogenase 21612 - | 1 . . . 418 | 418/418 | (100%) | |
| | Homo sapiens, 418 aa. | | | | |
| | [WO200144446-A2, 21 JUN 2001] | | | | |
| AAG81258 | Human AFP protein sequence SEQ | 1 . . . 382 | 382/382 | (100%) | 0.0 |
| | ID NO:34 - Homo sapiens, 383 aa. | 1 . . . 382 | 382/382 | (100%) | |
| | [WO200129221-A2, 26 APR | | | | |
| | 2001] | | | | |
| ABB10251 | Human cDNA SEQ ID NO: 559 - | 141 . . . 418 | 271/278 | (97%) | e−156 |
| | Homo sapiens, 278 aa. | 1 . . . 278 | 274/278 | (98%) | |
| | [WO200154474-A2, 2 AUG | | | | |
| | 2001] | | | | |
| AAU23020 | Novel human enzyme polypeptide | 141 . . . 418 | 271/278 | (97%) | e−156 |
| | #106 - Homo sapiens, 278 aa. | 1 . . . 278 | 274/278 | (98%) | |
| | [WO200155301-A2, 2 AUG | | | | |
| | 2001] | | | | |

In a BLAST search of public sequence databases, the NOV86a protein was found to have homology to the proteins shown in the BLASTP data in Table 86D.

TABLE 86D
Public BLASTP Results for NOV86a

| | | NOV86A | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| CAC38510 | SEQUENCE 37 FROM | 1 . . . 418 | 418/418 | (100%) | 0.0 |
| | PATENT WO0129221 - Homo | 1 . . . 418 | 418/418 | (100%) | |
| | sapiens (Human), 418 aa. | | | | |
| CAC38508 | SEQUENCE 33 FROM | 1 . . . 382 | 382/382 | (100%) | 0.0 |
| | PATENT WO0129221 - Homo | 1 . . . 382 | 382/382 | (100%) | |
| | sapiens (Human), 383 aa. | | | | |
| Q99LV2 | HYPOTHETICAL 54.9 KDA | 1 . . . 418 | 355/496 | (71%) | 0.0 |
| | PROTEIN - Mus musculus | 1 . . . 496 | 390/496 | (78%) | |
| | (Mouse), 496 aa. | | | | |
| Q9BT58 | SIMILAR TO RIKEN CDNA | 163 . . . 418 | 253/256 | (98%) | e−143 |
| | 2610207116 GENE - Homo | 90 . . . 345 | 254/256 | (98%) | |
| | sapiens (Human), 345 aa. | | | | |
| Q9VB10 | CG5590 PROTEIN (GH01709P) - | 4 . . . 418 | 238/422 | (56%) | e−128 |
| | Drosophila melanogaster (Fruit | 3 . . . 412 | 300/422 | (70%) | |
| | fly), 412 aa. | | | | |

PFam analysis predicts that the NOV86a protein contains the domains shown in the Table 86E.

TABLE 86E
Domain Analysis of NOV86a

| | | Identities/ | | |
|---|---|---|---|---|
| | | Similarities | | |
| | NOV86a Match | for the | | Expect |
| Pfam Domain | Region | Matched Region | | Value |
| beta-lactamase: | 222 . . . 236 | 4/15 | (27%) | 6.5 |
| domain 1 of 1 | | 14/15 | (93%) | |
| adh_short: | 9 . . . 321 | 74/339 | (22%) | 2.4e−29 |
| domain 1 of 1 | | 211/339 | (62%) | |
| SCP2: domain 1 of 1 | 306 . . . 415 | 41/114 | (36%) | 1.5e−25 |
| | | 87/114 | (76%) | |

### Example 87

The NOV87 clone was analyzed, and the nucleotide and predicted polypeptide sequences are shown in Table 87A.

TABLE 87A
NOV87 Sequence Analysis

| | |
|---|---|
| SEQ ID NO: 243 | 888 bp |

| | |
|---|---|
| NOV87a, | TTCAACAAGGGCCCCTCCTACAGGCTCTTGGCGGACGTCCAGAACAGGCTTCTGTTCA |
| CG59516-01 DNA Sequence | |
| | AATATGACTCCCAGAAGGAGGCAGAGCTCCGCAGCTGGATCAAGGGATTCACTGGCCT |
| | CTCCATCCGCCCCGACTTCCAGAAGGGCCTGAAGGACGGGATTATTTTATGCACACTC |
| | GTGAACAAACTGCAGCCGGGCTCAGTCCCCAAGATCAACGGCTTCCGTGTAGAACTGG |
| | CACCAGCTAGAAAACCTCTCCAACATCCTCAAGGCAATGGTCAGCTACGGCATGATCC |
| | CGTGGACCTATTTGAGGCCAACGACCTGTTTGAGAGTGGGAACAATATGCAGGTGCGG |
| | GTGTCTCTTCTCGCCCTGGCAGGGAAGGCCAAGACTAAGGGGCTGCAGAGCGGGGTGG |
| | ACATCCGTGACAAGTACTCAGAGAAGCAGAACTTCAACGACACCACCATGAAGGCCAG |
| | GCTGTGCGTCATCCGGCTGCAGATTACCAACAAATGTGCCAGCCAGTCAGGCATGACC |
| | GCATACGTCACGAGGAGGCATCTCTACGACCCCAAGAACCGCATCCTGCCCCCCATGG |
| | ACAACTCGACCATCAGCCTCCGGATGGGTACAAACAAGTGCGCCAGCCAGGTGGGCAT |
| | GACGGCTCCCGGGAACCAGTGGCACATCTATGACACCAAGTTGGGAATCGACAAGTGT |
| | GAGAACTCCTCCATGTCCCTGAAGATGGGCTACACGCAGGTCGCCAATCACAGCAGAC |
| | AGGTCTTTGGCCTAGGCCGGCAAATATATGAACCCAAGTACCAGCCGGGTGGCCCAGT |
| | GGCCCACGGGGCTCCCTCCGCCGGCAACTGCCCAGGGCCAGGGGAGGCCCCTTAGTAC |
| | CAGGAGGAGACCAGCTAC |

| | |
|---|---|
| ORF Start: TTC at 1 | ORF Stop: TAG at 865 |

| | | |
|---|---|---|
| SEQ ID NO: 244 | 288 aa | MW at 31831.2 kD |

| | |
|---|---|
| NOV87a, | FNKGPSYRLLADVQNRLLFKYDSQKEAELRSWIKGFTGLSIRPDFQKGLKDGIILCTL |
| CG59516-01 Protein Sequence | |
| | VNKLQPGSVPKINGFRVELAPARKPLQHPQGNGQLRHDPVDLFEANDLFESGNNMQVR |
| | VSLLALAGKAKTKGLQSGVDIRDKYSEKQNFNDTTMKARLCVIRLQITNKCASQSGMT |
| | AYVTRRHLYDPKNRILPPMDNSTISLRMGTNKCASQVGMTAPGNQWHIYDTKLGIDKC |
| | ENSSMSLKMGYTQVANHSRQVFGLGRQIYEPKYQPGGPVAHGAPSAGNCPGPGEAP |

| | |
|---|---|
| SEQ ID NO: 245 | 888 bp |

| | |
|---|---|
| NOV87b, | TTCAACAAGGGCCCCTCCTACAGGCTCTTGGCGGACGTCCAGAACAGGCTTCTGTTCA |
| CG59516-02 DNA Sequence | |
| | AATATGACTCCCAGAAGGAGGCAGAGCTCCGCAGCTGGATCAAGGGATTCACTGGCCT |
| | CTCCATCCGCCCCGACTTCCAGAAGGGCCTGAAGGACGGGATTATTTTATGCACACTC |
| | GTGAACAAACTGCAGCCGGGCTCAGTCCCCAAGATCAACGGCTTCCGTGTAGAACTGG |
| | CACCAGCTAGAAAACCTCTCCAACATCCTCAAGGCAATGGTCAGCTACGGCATGATCC |
| | CGTGGACCTATTTGAGGCCAACGACCTGTTTGAGAGTGGGAACAATATGCAGGTGCGG |
| | GTGTCTCTTCTCGCCCTGGCAGGGAAGGCCAAGACTAAGGGGCTGCAGAGCGGGGTGG |
| | ACATCCGTGACAAGTACTCAGAGAAGCAGAACTTCAACGACACCACCATGAAGGCCAG |
| | GCTGTGCGTCATCCGGCTGCAGATTACCAACAAATGTGCCAGCCAGTCAGGCATGACC |
| | GCATACGTCACGAGGAGGCATCTCTACGACCCCAAGAACCGCATCCTGCCCCCCATGG |
| | ACAACTCGACCATCAGCCTCCGGATGGGTACAAACAAGTGCGCCAGCCAGGTGGGCAT |
| | GACGGCTCCCGGGAACCAGTGGCACATCTATGACACCAAGTTGGGAATCGACAAGTGT |
| | GAGAACTCCTCCATGTCCCTGAAGATGGGCTACACGCAGGTCGCCAATCACAGCAGAC |
| | AGGTCTTTGGCCTAGGCCGGCAAATATATGAACCCAAGTACCAGCCGGGTGGCCCAGT |
| | GGCCCACGGGGCTCCCTCCGCCGGCAACTGCCCAGGGCCAGGGGAGGCCCCTTAGTAC |
| | CAGGAGGAGACCAGCTAC |

| | |
|---|---|
| ORF Start: TTC at 1 | ORF Stop: TAG at 865 |

| | | |
|---|---|---|
| SEQ ID NO: 246 | 288 aa | MW at 31831.2 kD |

| | |
|---|---|
| NOV87b, | FNKGPSYRLLADVQNRLLFKYDSQKEAELRSWIKGFTGLSIRPDFQKGLKDGIILCTL |
| CG59516-02 Protein Sequence | |
| | VNKLQPGSVPKINGFRVELAPARKPLQHPQGNGQLRHDPVDLFEANDLFESGNNMQVR |
| | VSLLALAGKAKTKGLQSGVDIRDKYSEKQNFNDTTMKARLCVIRLQITNKCASQSGMT |
| | ENSSMSLKMGYTQVANHSRQVFGLGRQIYEPKYQPGGPVAHGAPSAGNCPGPGEAP |

Sequence comparison of the above protein sequences yields the following sequence relationships shown in Table 87B.

TABLE 87B
Comparison of NOV87a against NOV87b.

| | | Identities/ |
|---|---|---|
| | | Similarities |
| Protein | NOV87a Residues/ | for the |
| Sequence | Match Residues | Matched Region |
| NOV87b | 1 . . . 288 | 288/288 (100%) |
| | 1 . . . 288 | 288/288 (100%) |

Further analysis of the NOV87a protein yielded the following properties shown in Table 87C.

TABLE 87C
Protein Sequence Properties NOV87a

| | |
|---|---|
| PSort | 0.4500 probability located in cytoplasm; 0.3000 probability |
| analysis: | located in microbody (peroxisome); 0.2110 probability |
| | located in lysosome (lumen); 0.1000 probability located in |
| | mitochondrial matrix space |
| SignalP | No Known Signal Sequence Predicted |
| analysis: | |

A search of the NOV87a protein against the Geneseq database, a proprietary database that contains sequences published in patents and patent publication, yielded several homologous proteins shown in Table 87D.

TABLE 87D
Geneseq Results for NOV87a

| | | NOV87a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAR94888 | Carponin - Homo sapiens, 297 aa. | 1 . . . 265 | 136/269 | (50%) | 7e−63 |
| | [JP08073380-A, 19 MAR 1996] | 6 . . . 272 | 176/269 | (64%) | |
| AAR72588 | Carponin protein - Homo sapiens, | 1 . . . 265 | 136/269 | (50%) | 7e−63 |
| | 297 aa. [WO9509010-A, 6 APR | 6 . . . 272 | 176/269 | (64%) | |
| | 1995] | | | | |
| AAB43807 | Human cancer associated protein | 164 . . . 273 | 67/113 | (59%) | 6e−30 |
| | sequence SEQ ID NO:1252 - Homo | 4 . . . 116 | 82/113 | (72%) | |
| | sapiens, 163 aa. [WO200055350- | | | | |
| | A1, 21 SEP 2000] | | | | |
| AAM73074 | Human bone marrow expressed | 157 . . . 225 | 49/70 | (70%) | 4e−21 |
| | probe encoded protein SEQ ID NO: | 2 . . . 71 | 55/70 | (78%) | |
| | 33380 - Homo sapiens, 71 aa. | | | | |
| | [WO200157276-A2, 09 AUG 2001] | | | | |
| AAM60434 | Human brain expressed single exon | 157 . . . 225 | 49/70 | (70%) | 4e−21 |
| | probe encoded protein SEQ ID NO: | 2 . . . 71 | 55/70 | (78%) | |
| | 32539 - Homo sapiens, 71 aa. | | | | |
| | [WO200157275-A2, 9 AUG 2001] | | | | |

In a BLAST search of public sequence databases, the NOV87a protein was found to have homology to the proteins shown in the BLASTP data in Table 87E.

TABLE 87E
Public BLASTP Results for NOV87a

| | | NOV87A | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q08094 | Calponin H2, smooth muscle - Sus | 1 . . . 287 | 219/291 (75%) | e−116 |
| | scrofa (Pig), 296 aa (fragment). | 6 . . . 296 | 237/291 (81%) | |
| Q99439 | Calponin H2, smooth muscle | 1 . . . 288 | 218/292 (74%) | e−115 |
| | (Neutral calponin) - Homo sapiens | 6 . . . 297 | 235/292 (79%) | |
| | (Human), 309 aa. | | | |
| Q08093 | Calponin H2, smooth muscle - | 1 . . . 288 | 214/291 (73%) | e−112 |
| | Mus musculus (Mouse), 305 aa. | 6 . . . 293 | 231/291 (78%) | |
| O93547 | CALPONIN H3 - Xenopus laevis | 1 . . . 269 | 179/273 (65%) | 6e−91 |
| | (African clawed frog), 295 aa. | 5 . . . 276 | 208/273 (75%) | |
| Q922F8 | UNKNOWN (PROTEIN FOR | 59 . . . 288 | 166/233 (71%) | 8e−83 |
| | MGC:8135) - Mus musculus | 1 . . . 230 | 179/233 (76%) | |
| | (Mouse), 242 aa. | | | |

PFam analysis predicts that the NOV87a protein contains the domains shown in the Table 87F.

TABLE 87F
Domain Analysis of NOV87a

| | | Identities/ | | |
|---|---|---|---|---|
| | | Similarities | | |
| | NOV87a Match | for the | | Expect |
| Pfam Domain | Region | Matched Region | | Value |
| CH: domain 1 of 1 | 23 . . . 123 | 27/124 | (22%) | 0.068 |
| | | 65/124 | (52%) | |
| calponin: | 159 . . . 183 | 17/26 | (65%) | 3.8e−07 |
| domain 1 of 2 | | 21/26 | (81%) | |
| calponin: | 198 . . . 223 | 15/26 | (58%) | 3e−08 |
| domain 2 of 2 | | 19/26 | (73%) | |

### Example 88

The NOV88 clone was analyzed, and the nucleotide and predicted polypeptide sequences are shown in Table 88A.

TABLE 88A
NOV 88 Sequence Analysis

| | |
|---|---|
| SEQ ID NO: 247 | 2213 bp |

| | |
|---|---|
| NOV88a, | CGTGAGGCACCCACTCTGGGAGCACAGAGAGCTCAGGTAGCCTGCCTAGATGGCGGCG |
| CG59671-02 DNA Sequence | |
| | CGCACCCTGGGCCGCGGCGTCGGGAGGCTGCTGGGCAGCCTGCGAGGGCTCTCGGGGC |
| | AGCCCGCGCGGCCGCCGTGCGGGGTGAGCGCGCCGCGCAGGGCGGCCTCGGGACCCTC |
| | GGGCAGCGCTCCCGCAGTTGCAGCAGCAGCAGCACAGCCAGGCTCGTATCCCGCGCTG |
| | AGTGCACAGGCAGCCCGGGAGCCGGCCGCCTTCTGGGGGCCTCTGGCGCGGGACACTC |
| | TCGTGTGGGACACCCCCTACCACACCGTCTGGGACTGCGACTTCAGCACTGGCAAGAT |
| | CGGCTGGTTCCTGGGAGGCCAGTTAAATGTCTCTGTCAACTGCTTGGACCAGCATGTT |
| | CGGAAGTCCCCCGAGAGCGTTGCTTTGATCTGGGAGCGCGATGAGCCTGGAACGGAAG |
| | TGAGGATCACCTACAGGGAACTACTGGAGACCACGTGCCGCCTGGCCAACACGCTGAA |
| | GAGGCATGGAGTCCACCGTGGGGACCGTGTTGCCATCTACATGCCCGTGTCCCCATTG |
| | GCTGTGGCAGCAATGCTGGCCTGTGCCAGGATCGGAGCTGTCCACACAGTCATCTTTG |
| | CTGGCTTCAGTGCAGAGTCCTTGGCTGGGAGGATCAATGATGCCAAGTGCAAGGTGGT |
| | TATCACCTTCAACCAAGGACTCCGGGGTGGGCGCGTGGTGGAGCTGAAGAAAATAGTG |
| | GATGAGGCTGTGAAGCACTGCCCCACCGTGCAGCATGTCCTGGTGGCTCACAGGACAG |
| | ACAACAAGGTCCACATGGGGGATCTGGACGTCCCGCTGGAGCAGGAAATGGCCAAGGA |
| | GGACCCTGTTTGCGCCCCAGAGAGCATGGGCAGTGAGGACATGCTCTTCATGCTGTAC |
| | ACCTCAGGGAGCACCGGAATGCCCAAGGGCATCGTCCATACCCAGGCAGGCTACCTGC |
| | TCTATGCCGCCCTGACTCACAAGCTTGTGTTTGACCACCAGCCAGGTGACATCTTTGG |
| | CTGTGTGGCCGACATCGGTTGGATTACAGGACACAGCTACGTGGTGTATGGGCCTCTC |
| | TGCAATGGTGCCACCAGCGTCCTTTTTGAGAGCACCCCAGTTTATCCCAATGCTGGTC |
| | GGTACTGGGAGACAGTAGAGAGGTTGAAGATCAATCAGTTCTATGGCGCCCCAACGGC |
| | TGTCCGGCTGTTGCTGAAATACGGTGATGCCTGGGTGAAGAAGTATGATCGCTCCTCC |
| | CTGCGGACCCTGGGGTCAGTGGGAGAGCCCATCAACTGTGAGGCCTGGGAGTGGCTTC |
| | ACAGGGTGGTGGGGGACAGCAGGTGCACGCTGGTGGACACCTGGTGGCAGACAGAAAC |
| | AGGTGGCATCTGCATCGCACCACGGCCCTCGGAAGAAGGGGCGGAAATCCTCCCTGCC |
| | ATGGCGATGAGGCCCTTCTTTGGCATCGTCCCCGTCCTCATGGATGAGAAGGGCAGCG |
| | TCGTGGAGGGCAGCAACGTCTCCGGGGCCCTGTGCATCTCCCAGGCCTGGCCGGGCAT |
| | GGCCAGGACCATCTATGGCGACCACCAGCGATTTGTGGACGCCTACTTCAAGGCCTAC |
| | CCAGGCTATTACTTCACTGGAGACGGGGCTTACCGAACTGAGGGCGGCTATTACCAGA |
| | TCACAGGGCGGATGGATGATGTCATCAACATCAGTGGCCACCGGCTGGGGACCGCAGA |
| | GATTGAGGACGCCATCGCCGACCACCCTGCAGTACCAGAAAGTGCTGTCATTGGCTAC |
| | CCCCACGACATCAAAGGAGAAGCTGCCTTTGCCTTCATTGTGGTGAAAGATAGTGCGG |
| | GTGACTCAGATGTGGTGGTGCAGGAGCTCAAGTCCATGGTGGCCACCAAGATCGCCAA |
| | ATATGCTGTGCCTGATGAGATCCTGGTGGTGAAACGTCTTCCAAAAACCAGGTCTGGG |
| | AAGGTCATGCGGCGGCTCCTGAGGAAGATCATCACTAGTGAGGCCCAGGAGCTGGGAG |
| | ACACTACCACCTTGGAGGACCCCAGCATCATCGCAGAGATCCTGAGTGTCTACCAGAA |
| | GTGCAAGGACAAGCAGGCTGCTGCTAAGTGAGCTGGCACCTTGTGGGGCTCTTGGGAT |
| | GGGCGGGCACCCAAGCCCTGGCTTGTCCTTCCCAGAAGGTACCCCTGAGGTTGGCGTC |
| | TTCCTACGT |

| | |
|---|---|
| ORF Start: ATG at 50 | ORF Stop: TGA at 2117 |

| | | |
|---|---|---|
| SEQ ID NO: 248 | 689 aa | MW at 74855.9 kD |

| | |
|---|---|
| NOV88a, | MAARTLGRGVGRLLGSLRGLSGQPARPPCGVSAPRRAASGPSGSAPAVAAAAAQPGSY |
| CG59671-02 Protein Sequence | |
| | PALSAQAAREPAAFWGPLARDTLVWDTPYHTVWDCDFSTGKIGWFLGGQLNVSVNCLD |
| | QHVRKSPESVALIWERDEPGTEVRITYRELLETTCRLANTLKRHGVHRGDRVAIYMPV |
| | SPLAVAAMLACARIGAVHTVIFAGFSAESLAGRINDAKCKVVITFNQGLRGGRVVELK |
| | KIVDEAVKHCPTVQHVLVAHRTDNKVHMGDLDVPLEQEMAKEDPVCAPESMGSEDMLF |
| | MLYTSGSTGMPKGIVHTQAGYLLYAALTHKLVFDHQPGDIFGCVADIGWITGHSYVVY |
| | GPLCNGATSVLFESTPVYPNAGRYWETVERLKINQFYGAPTAVRLLLKYGDAWVKKYD |
| | RSSLRTLGSVGEPINCEAWEWLHRVVGDSRCTLVDTWWQTETGGICIAPRPSEEGAEI |
| | LPAMAMRPFFGIVPVLMDEKGSVVEGSNVSGALCISQAWPGMARTIYGDHQRFVDAYF |
| | KAYPGYYFTGDGAYRTEGGYYQITGRMDDVINISGHRLGTAEIEDAIADHPAVPESAV |
| | IGYPHDIKGEAAFAFIVVKDSAGDSVVVQELKSMVATKIAKYAVPDEILVVKKRLPKT |
| | RSGKVMRRLLRKIITSEAQELGDTTTLEDPSIIAEILSVYQKCKDKQAAAK |

Further analysis of the NOV88a protein yielded the following properties shown in Table 88B.

TABLE 88B
Protein Sequence Properties NOV88a

| | |
|---|---|
| PSort | 0.6500 probability located in plasma membrane; 0.6000 |
| analysis: | probability located in nucleus; 0.4340 probability |
| | located in mitochondrial inner membrane; 0.3000 |
| | probability located in Golgi body |
| SignalP | Likely cleavage site between residues 23 and 24 |
| analysis: | |

A search of the NOV88a protein against the Geneseq database, a proprietary database that contains sequences published in patents and patent publication, yielded several homologous proteins shown in Table 88C.

TABLE 88C
Geneseq Results for NOV88a

| | | NOV88a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAU23058 | Novel human enzyme polypeptide | 26 . . . 689 | 663/664 | (99%) | 0.0 |
| | #144 - Homo sapiens, 664 aa. | 1 . . . 664 | 663/664 | (99%) | |
| | [WO200155301-A2, 2 AUG | | | | |
| | 2001] | | | | |
| AAB34712 | Human secreted protein encoded | 172 . . . 689 | 518/518 | (100%) | 0.0 |
| | by DNA clone vo9 1 - Homo | 1 . . . 518 | 518/518 | (100%) | |
| | sapiens, 518 aa. [WO200055375- | | | | |
| | A1, 21 SEP 2000] | | | | |
| AAU23050 | Novel human enzyme polypeptide | 224 . . . 689 | 459/466 | (98%) | 0.0 |
| | #136 - Homo sapiens, 479 aa. | 18 . . . 479 | 461/466 | (98%) | |
| | [WO200155301-A2, 2 AUG | | | | |
| | 2001] | | | | |
| ABB12253 | Human acetate-coA ligase | 1 . . . 446 | 446/446 | (100%) | 0.0 |
| | homologue, SEQ ID NO:2623 - | 1 . . . 446 | 446/446 | (100%) | |
| | Homo sapiens, 446 aa. | | | | |
| | [WO200157188-A2, 9 AUG | | | | |
| | 2001] | | | | |
| AAR23968 | facA gene product - Penicillium | 58 . . . 684 | 305/629 | (48%) | e−175 |
| | chrysogenum, 669 aa. | 45 . . . 669 | 407/629 | (64%) | |
| | [WO9207079-A, 30 APR 1992] | | | | |

In a BLAST search of public sequence databases, the NOV88a protein was found to have homology to the proteins shown in the BLASTP data in Table 88D.

TABLE 88D
Public BLASTP Results for NOV88a

| | | NOV88A | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| Q99NB1 | ACETYL-COA SYNTHETASE 2 - | 1 . . . 687 | 599/687 | (87%) | 0.0 |
| | Mus musculus (Mouse), 682 aa. | 1 . . . 680 | 638/687 | (92%) | |
| Q9BEA3 | ACETYL-COA SYNTHETASE 2 - | 1 . . . 689 | 575/689 | (83%) | 0.0 |
| | Bos taurus (Bovine), 675 aa. | 1 . . . 675 | 625/689 | (90%) | |
| Q9NUB1 | DJ568C11.3 (NOVEL AMP- | 212 . . . 689 | 478/478 | (100%) | 0.0 |
| | BINDING ENZYME SIMILAR TO | 1 . . . 478 | 478/478 | (100%) | |
| | ACETYL-COENZYME A | | | | |
| | LIGASE)) - Homo sapiens (Human), | | | | |
| | 478 aa (fragment). | | | | |
| Q96JI1 | KIAA1846 PROTEIN - Homo | 336 . . . 689 | 354/354 | (100%) | 0.0 |
| | sapiens (Human), 354 aa (fragment). | 1 . . . 354 | 354/354 | (100%) | |
| Q9HV66 | ACETYL-COENZYME A | 58 . . . 675 | 326/619 | (52%) | 0.0 |
| | SYNTHETASE - Pseudomonas | 24 . . . 639 | 433/619 | (69%) | |
| | aeruginosa, 645 aa. | | | | |

PFam analysis predicts that the NOV88a protein contains the domains shown in the Table 88E.

TABLE 88E
Domain Analysis of NOV88a

| | | Identities/ | |
|---|---|---|---|
| | | Similarities | |
| | NOV88a Match | for the | Expect |
| Pfam Domain | Region | Matched Region | Value |
| AMP-binding: | 142 . . . 580 | 121/441 (27%) | 7.1e−117 |
| domain 1 of 1 | | 341/441 (77%) | |

### Example 89

The NOV89 clone was analyzed, and the nucleotide and predicted polypeptide sequences are shown in Table 89A.

TABLE 89A
NOV89 Sequence Analysis

| | |
|---|---|
| SEQ ID NO: 249 | 1268 bp |

| | |
|---|---|
| NOV89a, | ACTTCTTTCTTTTCTGTTTCAGAGTTACTGATTTATTCTTGAGATTCCTCTACTCTCG |
| CG56870-01 DNA Sequence | |
| | TTATCTGACCTCATGGATGAACTTCAGGATGTTCAGCTCACAGAGATCAAACCACTTC |
| | TAAATGATAAGAATGGTACAAGAAACTTCCAGGACTTTGACTGTCAGGAACATGATAT |
| | AGAAACAACTCATGGTGTGGTCCACGTCACTATAAGAGGCTTACCCAAAGGAAACAGA |
| | CCAGTTATACTAACATATCATGACATTGGCCTCAACCGTAAATCCTGTTTCAATGCAT |
| | TCTTTAACTTTGAGGATATGCAAGAGATCACCCAGCACTTTGCTGTCTGTCATGTGGA |
| | TGCCCCAGGCCAGCAGGAAGGTGCACCCTCTTTCCCAACAGGGTATCAGTACCCCACA |
| | ATGGATGAGCTGGCTGAAATGCTGCCTCCTGTTCTTACCCACCTAAGCCTGAAAAGCA |
| | TCATTGGAATTGGAGTTGGAGCTGGAGCTTACATCCTCAGCAGATTTGCACTCAACCA |
| | TCCAGAGCTTGTGGAAGGCCTTGTGCTCATTAATGTTGACCCTTGCGCTAAAGGCTGG |
| | ATTGACTGGGCAGCTTCCAAACTCTCTGGCCTGACAACCAATGTTGTGGACATTATTT |
| | TGGCTCATCACTTTGGGCAGGAAGAGTTACAGGCCAACCTGGACCTGATCCAAACCTA |
| | CAGAATGCATATTGCCCAAGACATCAACCAAGACAACCTGCAGCTCTTCTTGAATTCC |
| | TACAATGGGCGCAGAGACCTGGAGATCGAAAGACCCATACTGGGCCAAAATGATAACA |
| | AATCAAAAACATTAAAGTGTTCTACTTTACTGGTGGTAGGGGACAATTCGCCTGCAGT |
| | TGAGGCTGTGGTCGAATGCAATTCCCGCCTGAACCCTATAAATACAACTTTGCTAAAG |
| | ATGGCGGACTGTGGGGGACTGCCCCAGGTAGTTCAGCCTGGGAAGCTCACCGAGGCCT |
| | TCAAGTACTTTTTGCAGGGAATGGGCTACGTCCCGTCTGCCAGCATGACTCGGCTCGC |
| | CCGATCACGAACCCACTCAACCTCGAGTAGCCTCGGCTCTGGAGAAAGTCCCTTCAGC |
| | CGGTCTGTCACCAGCAATCAGTCAGATGGAACTCAAGAATCCTGTGAGTCCCCTGATG |
| | TCCTGGACAGACACCAGACCATGGAGGTGTCCTGCTAAGCAGATGCTCCTCCCCTGGA |
| | CCATTGCAAGTCCATCCTTCAAATGACCACTCCATAATATAACATTTCAT |

| | |
|---|---|
| ORF Start: ATG at 71 | ORF Stop: TAA at 1196 |

| | | |
|---|---|---|
| SEQ ID NO: 250 | 375 aa | MW at 41413.3 kD |

| | |
|---|---|
| NOV89a, | MDELQDVQLTEIKPLLNDKNGTRNFQDFDCQEHDIETTHGVVHVTIRGLPKGNRPVIL |
| CG56870-01 Protein Sequence | |
| | TYHDIGLNRKSCFNAFFNFEDMQEITQHFAVCHVDAPGQQEGAPSFPTGYQYPTMDEL |
| | AEMLPPVLTHLSLKSIIGIGVGAGAYILSRFALNHPELVEGLVLINVDPCAKGWIDWA |
| | ASKLSGLTTNVVDIILAHHFGQEELQANLDLIQTYRMHIAQDINQDNLQLFLNSYNGR |
| | RDLEIERPILGQNDNKSKTLKCSTLLVVGDNSPAVEAVVECNSRLNPINTTLLKMADC |
| | GGLPQVVQPGKLTEAFKYFLQGMGYVPSASMTRLARSRTHSTSSSLGSGESPFSRSVT |
| | SNQSDGTQESCESPDVLDRHQTMEVSC |

| | |
|---|---|
| SEQ ID NO: 251 | 1175 bp |

| | |
|---|---|
| NOV89b, | TCGTTATCTGACCTCATGGATGAACTTCAGGATGTTCAGCTCACAGAGATCAAACCAC |
| CG56870-02 DNA Sequence | |
| | TTCTAAATGATAAGAATGGTACAAGAAACTTCCAGGACTTTGACTGTCAGGAACATGA |
| | TATAGAAACAACTCATGGTGTGGTCCACGTCACTATAAGAGGCTTACCCAAAGGAAAC |
| | AGACCAGTTATACTAACATATCATGACATTGGCCTCAACCATAAATCCTGTTTCAATG |
| | CATTCTTTAACTTTGAGGATATGCAAGAGATCACCCAGCACTTTGCTGTCTGTCATGT |
| | GGATGCCCCAGGCCAGCAGGAAGGTGCACCCTCTTTCCCAACAGGGTATCAGTACCCC |
| | ACAATGGATGAGCTGGCTGAAGTGCTGCCTCCTGTTCTTACCCACCTAAGCCTGAAAA |
| | GCATCATTGGAATTGGAGTTGGAGCTGGAGCTTACATCCTCAGCAGATTTGCACTCAA |
| | CCATCCAGAGCTTGTGGAAGGCCTTGTGCTCATTAATGTTGACCCTTGCGCTAAAGGC |
| | TGGATTGACTGGGCAGCTTCCAAACTCTCTGGCCTGACAACCAATGTTGTGGACATTA |
| | TTTTGGCTCATCACTTTGGGCAGGAAGAGTTACAGGCCAACCTGGACCTGATCCAAAC |
| | CTACAGAATGCATATTGCCCAAGACATCAACCAAGACAACCTGCAGCTCTTCTTGAAT |
| | TCCTACAATGGACGCAGAGACCTGGAGATCGAAAGACCCATACTGGGCCAAAATGATA |
| | ACAAATCAAAAACATTAAAGTGTTCTACTTTACTGGTGGTAGGGGACAATTCGCCTGC |
| | AGTTGAGGCTGTGGTCGAATGCAATTCCCGCCTGAACCCTATAAATACAACTTTGCTA |
| | AAGATGGCGGACTGTGGGGGACTGCCCCAGGTAGTTCAGCCTGGGAAGCTCACCGAGG |
| | CCTTCAAGTACTTTTTGCAGGGAATGGGCTACATACCATCTGCCAGCATGACTCGGCT |
| | CGCCCGATCACGAACCCACTCAACCTCGAGTAGCCTCGGCTCTGGAGAAAGTCCCTTC |
| | AGCCGGTCTGTCACCAGCAATCAGTCAGATGGAACTCAAGAATCCTGTGAGTCCCCTG |
| | ATGTCCTGGACAGACACCAGACCATGGAGGTGTCCTGCTAAGCAGATGCTCCTCCCCT |
| | GGACCATTGCAAGTC |

| | |
|---|---|
| ORF Start: ATG at 16 | ORF Stop: TAA at 1141 |

| | | |
|---|---|---|
| SEQ ID NO: 252 | 375 aa | MW at 41376.2 kD |

| | |
|---|---|
| NOV89b, | MDELQDVQLTEIKPLLNDKNGTRNFQDFDCQEHDIETTHGVVHVTIRGLPKGNRPVIL |
| CG56870-02 Protein Sequence | |
| | TYHDIGLNHKSCFNAFFNFEDMQEITQHFAVCHVDAPGQQEGAPSFPTGYQYPTMDEL |
| | AEVLPPVLTHLSLKSIIGIGVGAGAYILSRFALNHPELVEGLVLINVDPCAKGWIDWA |
| | ASKLSGLTTNVVDIILAHHFGQEELQANLDLIQTYRMHIAQDINQDNLQLFLNSYNGR |
| | RDLEIERPILGQNDNKSKTLKCSTLLVVGDNSPAVEAVVECNSRLNPINTTLLKMADC |
| | GGLPQVVQPGKLTEAFKYFLQGMGYIPSASMTRLARSRTHSTSSSLGSGESPFSRSVT |
| | SNQSDGTQESCESPDVLDRHQTMEVSC |

| | |
|---|---|
| SEQ ID NO: 253 | 1232 bp |

| | |
|---|---|
| NOV89c, | ACTTCTTTCTTTTCTGTTTCAGAGTTACTGATTTATTCTTGAGATTCCTCTACTCTCG |
| CG56870-03 DNA Sequence | |
| | TTATCTGACCTCATGGATGAACTTCAGGATGTTCAGCTCACAGAGATCAAACCACTTC |
| | TAAATGATAAGGAACATGATATAGAAACAACTCATGGTGTGGTCCACGTCACTATAAG |
| | AGGCTTACCCAAAGGAAACAGACCAGTTATACTAACATATCATGACATTGGCCTCAAC |
| | CATAAATCCTGTTTCAATGCATTCTTTAACTTTGAGGATATGCAAGAGATCACCCAGC |
| | ACTTTGCTGTCTGTCATGTGGATGCCCCAGGCCAGCAGGAAGGTGCACCCTCTTTCCC |
| | AACAGGGTATCAGTACCCCACAATGGATGAGCTGGCTGAAATGCTGCCTCCTGTTCTT |
| | ACCCACCTAAGCCTGAAAAGCATCATTGGAATTGGAGTTGGAGCTGGAGCTTACATCC |
| | TCAGCAGATTTGCACTCAACCATCCAGAGCTTGTGGAAGGCCTTGTGCTCATTAATGT |
| | TGACCCTTGCGCTAAAGGCTGGATTGACTGGGCAGCTTCCAAACTCTCTGGCCTGACA |
| | ACCAATGTTGTGGACATTATTTTGGCTCATCACTTTGGGCAGGAAGAGTTACAGGCCA |
| | ACCTGGACCTGATCCAAACCTACAGAATGCATATTGCCCAAGACATCAACCAAGACAA |
| | CCTGCAGCTCTTCTTGAATTCCTACAATGGGCGCAGAGACCTGGAGATCGAAAGACCC |
| | ATACTGGGCCAAAATGATAACAAATCAAAAACATTAAAGTGTTCTACTTTACTGGTGG |
| | TAGGGGACAATTCGCCTGCAGTTGAGGCTGTGGTCGAATGCAATTCCCGCCTGAACCC |
| | TATAAATACAACTTTGCTAAAGATGGCGGACTGTGGGGGACTGCCCCAGGTAGTTCAG |
| | CCTGGGAAGCTCACCGAGGCCTTCAAGTACTTTTTGCAGGGAATGGGCTACGTCCCGT |
| | CTGCCAGCATGACTCGGCTCGCCCGATCACGAACCCACTCAACCTCGAGTAGCCTCGG |
| | CTCTGGAGAAAGTCCCTTCAGCCGGTCTGTCACCAGCAATCAGTCAGATGGAACTCAA |
| | GAATCCTGTGAGTCCCCTGATGTCCTGGACAGACACCAGACCATGGAGGTGTCCTGCT |
| | AAGCAGATGCTCCTCCCCTGGACCATTGCAAGTCCATCCTTCAAATGACCACTCCATA |
| | ATATAACATTTCAT |

| | |
|---|---|
| ORF Start: ATG at 71 | ORF Stop: TAA at 1160 |

| | | |
|---|---|---|
| SEQ ID NO: 254 | 363 aa | MW at 39967.8 kD |

| | |
|---|---|
| NOV89c, | MDELQDVQLTEIKPLLNDKEHDIETTHGVVHVTIRGLPKGNRPVILTYHDIGLNHKSC |
| CG56870-03 Protein Sequence | |
| | FNAFFNFEDMQEITQHFAVCHVDAPGQQEGAPSFPTGYQYPTMDELAEMLPPVLTHLS |
| | LKSEEGIGVGAGAYILSRFALNHPELVEGLVLINVDPCAKGWIDWAASKLSGLTTNVV |
| | DIILAHHFGQEELQANLDLIQTYRMHIAQDINQDNLQLFLNSYNGRRDLEIERPILGQ |
| | NDNKSKTLKCSTLLVVGDNSPAVEAVVECNSRLNPINTTLLKMADCGGLPQVVQPGKL |
| | TEAFKYFLQGMGYVPSASMTRLARSRTHSTSSSLGSGESPFSRSVTSNQSDGTQESCE |
| | SPDVLDRHQTMEVSC |

| | |
|---|---|
| SEQ ID NO: 255 | 1220 bp |

| | |
|---|---|
| NOV89d, | ACTTCTTTCTTTTCTGTTTCAGAGTTACTGATTTATTCTTGAGATTCCTCTACTCTCG |
| CG56870-04 | DNA Sequence |
| | TTATCTGACCTCATGGATGAACTTCAGGATGTTCAGCTCACAGAGATCAAACCACTTC |
| | TAAATGATAAGAATGGTACAAGAAACTTCCAGGACTTTGACTGTCAGGAACATGATAT |
| | AGAAACAACTCATGGTGTGGTCCACGTCACTATAAGAGGCTTACCCAAAGGAAACAGA |
| | CCAGTTATACTAACATATCATGACATTGGCCTCAACCGTAAATCCTGTTTCAATGCAT |
| | TCTTTAACTTTGAGGATATGCAAGAGATCACCCAGCACTTTGCTGTCTGTCATGTGGA |
| | TGCCCCAGGCCAGCAGGAAGGTGCACCCTCTTTCCCAACAGGGTATCAGTACCCCACA |
| | ATGGATGAGCTGGCTGAAATGCTGCCTCCTGTTCTTACCCACCTAAGCCTGAAAAGCA |
| | TCATTGGAATTGGAGTTGGAGCTGGAGCTTACATCCTCAGCAGATTTGCACTCAACCA |
| | TCCAGAGCTTGTGGAAGGCCTTGTGCTCATTAATGTTGACCCTTGCGCTAAAGGCTGG |
| | ATTGACTGGGCAGCTTCCAAACTCTCTGGCCTGACAACCAATGTTGTGGACATTATTT |
| | TGGCTCATCACTTTGGGCAGGAAGAGTTACAGGCCAACCTGGACCTGATCCAAACCTA |
| | CAGAATGCATATTGCCCAAGACATCAACCAAGACAACCTGCAGCTCTTCTTGAATTCC |
| | TACAATGGACGCAGAGACCTGGAGATCGAAAGACCCATACTGGGCCAAAATGATAACA |
| | AATCAAAAACATTAAAGTGTTCTACTTTACTGGTGGTAGGGGACAATTCGCCTGCAGT |
| | TGAGGCTGTGATGGCGGACTGTGGGGGACTGCCCCAGGTAGTTCAGCCTGGGAAGTTC |
| | ACCGAGGCCTTCAAGTACTTTTTGCAGGGAATGGGCTACACACCATCTGCCAGCATGA |
| | CTCGGCTCGCCCGATCACGAACCCACTCAACCTCGAGTAGCCTCGGCTCTGGAGAAAG |
| | TCCCTTCAGCCGGTCTGTCACCAGCAATCAGTCAGATGGAACTCAAGAATCCTGTGAG |
| | TCCCCTGATGTCCTGGACAGACACCAGACCATGGAGGTGTCCTGCTAAGCAGATGCTC |
| | CTCCCCTGGACCATTGCAAGTCCATCCTTCAAATGACCACTCCATAATATAACATTTC |
| | AT |

| | |
|---|---|
| ORF Start: ATG at 71 | ORF Stop: TAA at 1148 |

| | | |
|---|---|---|
| SEQ ID NO: 256 | 359 aa | MW at 39652.2 kD |

| | |
|---|---|
| NOV89d, | MDELQDVQLTEIKPLLNDKNGTRNFQDFDCQEHDIETTHGVVHVTIRGLPKGNRPVIL |
| CG56870-04 Protein Sequence | |
| | TYHDIGLNRKSCFNAFFNFEDMQEITQHFAVCHVDAPGQQEGAPSFPTGYQYPTMDEL |
| | AEMLPPVLTHLSLKSIIGIGVGAGAYILSRFALNHPELVEGLVLINVDPCAKGWIDWA |
| | ASKLSGLTTNVVDIILAHHFGQEELQANLDLIQTYRMHIAQDINQDNLQLFLNSYNGR |
| | RDLEIERPILGQNDNKSKTLKCSTLLVVGDNSPAVEAVMADCGGLPQVVQPGKFTEAF |
| | KYFLQGMGYTPSASMTRLARSRTHSTSSSLGSGESPFSRSVTSNQSDGTQESCESPDV |
| | LDRHQTMEVSC |

| | |
|---|---|
| SEQ ID NO: 257 | 970 bp |

| | |
|---|---|
| NOV89e, | ATGGATGAACTTCAGGATGTTCAGCTCACAGAGATCAAACCACTTCTAAATGATAAGA |
| CG56870-05 DNA Sequence | |
| | ATGGTACAAGAAACTTCCAGGACTTTGACTGTCAGTATCAGTACCCCACAATGGATGA |
| | GCTGGCTGAAATGCTGCCTCCTGTTCTTACCCACCTAAGCCTGAAAAGCATCATTGGA |
| | ATTGGAGTTGGAGCTGGAGCTTACATCCTCAGCAGATTTGCACTCAACCATCCAGAGC |
| | TTGTGGAAGGCCTTGTGCTCATTAATGTTGACCCTTGCGCTAAAGGCTGGATTGACTG |
| | GGCAGCTTCCAAACTCTCTGGCCTGACAACCAATGTTGTGGACATTATTTTGGCTCAT |
| | CACTTTGGGCAGGAAGAGTTACAGGCCAACCTGGACCTGATCCAAACCTACAGAATGC |
| | ATATTGCCCAAGACATCAACCAAGACAACCTGCAGCTCTTCTTGAATTCCTACAATGG |
| | GCGCAGAGACCTGGAGATCGAAAGACCCATACTGGGCCAAAATGATAACAAATCAAAA |
| | ACATTAAAGTGTTCTACTTTACTGGTGGTAGGGGACAATTCGCCTGCAGTTGAGGCTG |
| | TGGTCGAATGCAATTCCCGCCTGAACCCTATAAATACAACTTTGCTAAAGATGGCGGA |
| | CTGTGGGGGACTGCCCCAGGTAGTTCAGCCTGGGAAGCTCACCGAGGCCTTCAAGTAC |
| | TTTTTGCAGGGAATGGGCTACGTCCCGTCTGCCAGCATGACTCGGCTCGCCCGATCAC |
| | GAACCCACTCAACCTCGAGTAGCCTCGGCTCTGGAGAAAGTCCCTTCAGCCGGTCTGT |
| | CACCAGCAATCAGTCAGATGGAACTCAAGAATCCTGTGAGTCCCCTGATGTCCTGGAC |
| | AGACACCAGACCATGGAGGTGTCCTGCTAAGCAGATGCTCCTCCCCTGGACCATTGCA |
| | AGTCCATCCTTCAAATGACCACTCCATAATATAACATTTCAT |

| | |
|---|---|
| ORF Start: ATG at 1 | ORF Stop: TAA at 898 |

| | | |
|---|---|---|
| SEQ ID NO: 258 | 299 aa | MW at 32956.9 kD |

| | |
|---|---|
| NOV89e, | MDELQDVQLTEIKPLLNDKNGTRNFQDFDCQYQYPTMDELAEMLPPVLTHLSLKSIIG |
| CG56870-05 Protein Sequence | |
| | IGVGAGAYILSRFALNHPELVEGLVLINVDPCAKGWIDWAASKLSGLTTNVVDIILAH |
| | HFGQEELQANLDLIQTYRMHIAQDINQDNLQLFLNSYNGRRDLEIERPILGQNDNKSK |
| | TLKCSTLLVVGDNSPAVEAVVECNSRLNPINTTLLKMADCGGLPQVVQPGKLTEAFKY |
| | FLQGMGYVPSASMTRLARSRTHSTSSSLGSGESPFSRSVTSNQSDGTQESCESPDVLD |

Sequence comparison of the above protein sequences yields the following sequence relationships shown in Table 89B.

TABLE 89B
Comparison of NOV89a against NOV89b through NOV89e.

| | | Identities/ |
|---|---|---|
| Protein | NOV89a Residues/ | Similarities for |
| Sequence | Match Residues | the Matched Region |
| NOV89b | 1 . . . 375 | 336/375 (89%) |
| | 1 . . . 375 | 338/375 (89%) |
| NOV89c | 1 . . . 375 | 326/375 (86%) |
| | 1 . . . 363 | 326/375 (86%) |
| NOV89d | 1 . . . 375 | 321/375 (85%) |
| | 1 . . . 359 | 321/375 (85%) |
| NOV89e | 104 . . . 375 | 233/272 (85%) |
| | 28 . . . 299 | 233/272 (85%) |

Further analysis of the NOV89a protein yielded the following properties shown in Table 89C.

TABLE 89C
Protein Sequence Properties NOV89a

| | |
|---|---|
| PSort | 0.4500 probability located in cytoplasm; 0.3000 probability |
| analysis: | located in microbody (peroxisome); 0.1685 probability located |
| | in lysosome (lumen); 0.1000 probability located in |
| | mitochondrial matrix space |
| SignalP | No Known Signal Sequence Predicted |
| analysis: | |

A search of the NOV89a protein against the Geneseq database, a proprietary database that contains sequences published in patents and patent publication, yielded several homologous proteins shown in Table 89D.

TABLE 89D
Geneseq Results for NOV89a

| | | NOV89a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM94019 | Human stomach cancer expressed | 1 . . . 375 | 360/375 (96%) | 0.0 |
| | polypeptide SEQ ID NO 108- | 1 . . . 363 | 361/375 (96%) | |
| | Homo sapiens, 363 aa. | | | |
| | [WO200109317-A1, 8 FEB. 2001] | | | |
| AAG64392 | Human reducing agent and | 1 . . . 375 | 360/375 (96%) | 0.0 |
| | tunicamycin-responsive protein 40- | 1 . . . 363 | 361/375 (96%) | |
| | Homo sapiens, 363 aa. | | | |
| | [WO200155375-A1, | | | |
| | 2 AUG. 2001] | | | |
| AAB94494 | Human protein sequence SEQ ID | 1 . . . 375 | 360/375 (96%) | |
| | NO: 15186-Homo sapiens, 363 aa. | 1 . . . 363 | 361/375 (96%) | |
| | [EP1074617-A2, 7 FEB. 2001] | | | |
| AAU31598 | Novel human secreted protein | 68 . . . 374 | 282/323 (87%) | e−154 |
| | #2089-Homo sapiens, 395 aa. | 1 . . . 307 | 286/323 (88%) | |
| | [WO200179449-A2, | | | |
| | 25 OCT. 2001] | | | |
| AAB95462 | Human protein sequence SEQ ID | 133 . . . 375 | 240/243 (98%) | e−138 |
| | NO: 17944-Homo sapiens, 286 aa. | 44 . . . 286 | 242/243 (98%) | |
| | [EP1074617-A2, 7 FEB. 2001] | | | |

In a BLAST search of public sequence databases, the NOV89a protein was found to have homology to the proteins shown in the BLASTP data in Table 89E.

TABLE 89E
Public BLASTP Results for NOV89a

| | | NOV89a | | |
|---|---|---|---|---|
| Protein | | Residues/ | Identities/ | |
| Accession | | Match | Similarities for | Expect |
| Numer | Protein/Organism/Length | Residues | the Matched Portion | Value |
| Q9UGV2 | NDRG3 protein-Homo sapiens | 1 . . . 375 | 373/375 (99%) | 0.0 |
| | (Human), 375 aa. | 1 . . . 375 | 374/375 (99%) | |
| Q96PL8 | NDR1-RELATED | 1 . . . 375 | 372/375 (99%) | 0.0 |
| | DEVELOPMENT PROTEIN NDR3- | 1 . . . 375 | 373/375 (99%) | |
| | Homo sapiens (Human), 375 aa. | | | |
| Q9QYF9 | NDRG3 protein (Ndr3 protein)- | 1 . . . 375 | 358/375 (95%) | 0.0 |
| | Mus musculus (Mouse), 375 aa. | 1 . . . 375 | 368/375 (97%) | |
| AAH18504 | SIMILAR TO N-MYC | 1 . . . 375 | 359/388 (92%) | 0.0 |
| | DOWNSTREAM REGULATED 3- | 1 . . . 388 | 368/388 (94%) | |
| | Mus musculus (Mouse), 388 aa. | | | |
| Q96SM2 | CDNA FLJ14759 FIS, CLONE | 1 . . . 375 | 360/375 (96%) | 0.0 |
| | NT2RP3003290, MODERATELY | 1 . . .363 | 361/375 (96%) | |
| | SIMILAR TO MUS MUSCULUS | | | |
| | NDR1 RELATED PROTEIN NDR3- | | | |
| | Homo sapiens (Human), 363 aa. | | | |

PFam analysis predicts that the NOV89a protein contains the domains shown in the Table 89F.

TABLE 89F
Domain Analysis of NOV89a

| | | Identities/ | |
|---|---|---|---|
| Pfam | NOV89a | Similarities for | Expect |
| Domain | Match Region | the Matched Region | Value |
| Orn_Arg_deC_N: | 62 . . . 89 | 7/33 (21%) | 1.9 |
| domain 1 of 1 | | 24/33 (73%) | |
| abhydrolase: | 87 . . . 310 | 48/239 (20%) | 0.0066 |
| domain 1 of 1 | | 142/239 (59%) | |
| Ndr: domain 1 of 1 | 22 . . . 346 | 210/340 (62%) | 3.7e−211 |
| | | 311/340 (91%) | |

### Example 90

The NOV90 clone was analyzed, and the nucleotide and predicted polypeptide sequences are shown in Table 90A.

TABLE 90A
NOV90 Sequence Analysis

| | |
|---|---|
| SEQ ID NO: 259 | 632 bp |

| | |
|---|---|
| NOV90a, | GAAACTATAAAGGGTCCGAACCCTCTTTTAAAGGATCCCAATGCATTTCTTTGATCCC |
| CG59764-01 DNA Sequence | |
| | TCGCCGGTGCGACGGTACCACCATCCCAGCTGTGAGGCTGCCATCAACACCCACATCA |
| | GCCTGGAGCTCCACGCATCCTATGTGTACCTGTCCATGGCCTTCTACTTCGACCAGGA |
| | CGACGCGGCCCTGGAGCACTTTGACCGCTACTTCCTGCGCCAGTCGCAGGAGAAAAGG |
| | GAGCACGCCCAGGAGCTGATGAGCCTGCAGAACCTGCGCGGTGGCCGCATCTGCCTTC |
| | ATGACATCAGGAAGCCAGAGGGCCAAGGCTGGGAGAGCGGGCTCAAGGCCATGGAGTG |
| | CACCTTCCACCTGGAGAAGAACATCAACCAGAGCCTCCTGGAGCTGCACCAGCTGGCC |
| | AGGGAGAACGGCGACCCCCAGCTCTGCGACTTCCTGGAGAACGACTTCCTGAACCAGC |
| | AGGCCAAGACCATCAAAGAGCTGGGTGGCTACCTGAGCAACCTGCACAAGATGGGGGC |
| | CCCGGAAGCAGGCCTGGCAGAGTACCTCTTTAACAAGCTCACCCTGGGCCGCAGCGAA |
| | CCACTTCCTTGAACCAGCAGGCCAAGACCATCAAAGAGATTGGTGGCTACCT |

| | |
|---|---|
| ORF Start: ATG at 41 | ORF Stop: TGA at 590 |

| | | |
|---|---|---|
| SEQ ID NO: 260 | 183 aa | MW at 21159.6 kD |

| | |
|---|---|
| NOV90a, | MHFFDPSPVRRYHHPSCEAAINTHISLELHASYVYLSMAFYFDQDDAALEHFDRYFLR |
| CG59764-01 Protein Sequence | |
| | QSQEKREHAQELMSLQNLRGGRICLHDIRKPEGQGWESGLKAMECTFHLEKNINQSLL |
| | ELHQLARENGDPQLCDFLENDFLNQQAKTIKELGGYLSNLHKMGAPEAGLAEYLFNKL |
| | TLGRSEPLP |

Further analysis of the NOV90a protein yielded the following properties shown in Table 90B.

TABLE 90B
Protein Sequence Properties NOV90a

| | |
|---|---|
| PSort | 0.4500 probability located in cytoplasm; 0.1400 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV90a protein against the Geneseq database, a proprietary database that contains sequences published in patents and patent publication, yielded several homologous proteins shown in Table 90C.

TABLE 90C
Geneseq Results for NOV90a

| | | NOV90a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU07889 | Polypeptide sequence for human | 7 . . . 180 | 159/174 (91%) | 4e−91 |
| | hspG34a-Homo sapiens, 221 aa. | 45 . . . 218 | 164/174 (93%) | |
| | [WO200166752-A2, | | | |
| | 13 SEP. 2001] | | | |
| AAU07890 | Polypeptide sequence for human | 6 . . . 177 | 125/172 (72%) | 6e−70 |
| | hspG34b-Homo sapiens, 183 aa. | 6 . . . 177 | 149/172 (85%) | |
| | [WO200166752-A2, | | | |
| | 13 SEP. 2001] | | | |
| AAB90804 | Human shear stress-response | 7 . . . 180 | 114/174 (65%) | 6e−64 |
| | protein SEQ ID NO: 108-Homo | 7 . . . 180 | 141/174 (80%) | |
| | sapiens, 183 aa. [WO200125427- | | | |
| | A1, 12 APR. 2001] | | | |
| AAR71567 | Human monocyte growth factor- | 7 . . . 180 | 114/174 (65%) | 6e−64 |
| | Homo sapiens, 183 aa. | 7 . . . 180 | 141/174 (80%) | |
| | [JP07031482-A, 3 FEB. 1995] | | | |
| AAU27741 | Mouse full-length polypeptide | 6 . . . 180 | 112/175 (64%) | 5e−63 |
| | sequence #66-Mus musculus, 182 | 6 . . . 180 | 141/175 (80%) | |
| | aa. [WO200164834-A2, | | | |
| | 7 SEP. 2001] | | | |

In a BLAST search of public sequence databases, the NOV90a protein was found to have homology to the proteins shown in the BLASTP data in Table 90D.

TABLE 90D
Public BLASTP Results for NOV90a

| | | NOV90a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9BXU8 | Ferritin heavy polypeptide-like 17- | 6 . . . 177 | 125/172 (72%) | 2e−69 |
| | Homo sapiens (Human), 183 aa. | 6 . . . 177 | 149/172 (85%) | |
| P29389 | Ferritin heavy chain (Ferritin H | 6 . . . 180 | 115/175 (65%) | 6e−64 |
| | subunit)-Cricetulus griseus | 10 . . . 184 | 142/175 (80%) | |
| | (Chinese hamster), 185 aa. | | | |
| A26886 | ferritin heavy chain-chicken, 180 | 6 . . . 180 | 112/175 (64%) | 1e−63 |
| | aa. | 5 . . . 179 | 142/175 (81%) | |
| P08267 | Ferritin heavy chain (Ferritin H | 6 . . . 180 | 112/175 (64%) | 1e−63 |
| | subunit)-Gallus gallus (Chicken), | 4 . . . 178 | 142/175 (81%) | |
| | 179 aa. | | | |
| Q95MP7 | FERRITIN-Canis familiaris | 6 . . . 180 | 112/175 (64%) | 2e−63 |
| | (Dog), 183 aa. | 6 . . . 180 | 143/175 (81%) | |

PFam analysis predicts that the NOV90a protein contains the domains shown in the Table 90E.

TABLE 90E
Domain Analysis of NOV90a

| | | Identities/ | |
|---|---|---|---|
| Pfam | NOV90a | Similarities for | Expect |
| Domain | Match Region | the Matched Region | Value |
| Bacteriofer: | 14 . . . 159 | 35/172 (20%) | 6.7 |
| domain 1 of 1 | | 76/172 (44%) | |
| ferritin: | 17 . . . 173 | 92/161 (57%) | 4.7e−87 |
| domain 1 of 1 | | 138/161 (86%) | |

### Example 91

The NOV91 clone was analyzed, and the nucleotide and predicted polypeptide sequences are shown in Table 91A.

TABLE 91A
NOV91 Sequence Analysis

| | |
|---|---|
| SEQ ID NO: 261 | 487 bp |

| | |
|---|---|
| NOV91a, | TGCTGTGCGTTGTCTTTCCTTCTCACTCAAGCCTGTGAAATCTCTCTTTCAGGTTGAC |
| CG59710-01 DNA Sequence | |
| | AGACTAATGGAGTTGCATTTTAAATATCTGGGTGCAATGCAGGTGGCGGACAAGAAGA |
| | TTGAAGGGGAAAAACACGACATGGTCCGGCGAGGAGAGATCATCGACAATGACACCGA |
| | GGAGGAGTTCTACCTCCGGCGCCTGGATGCGGGGCTCTTTGTTCTCCAGCACATCTGC |
| | TACATCATGGCCGAGATCTGCAATGCCAATGTCCCCCAGATTCGCCAGAGGGTTCACC |
| | AGATCCTAAACATGCGAGGAAGCTCCATCAAAATTGTCAGGCATATCATCAAGGAGTA |
| | TGCAGAGAACATCGGGGACGGCCGGAGCCCGGAGTTCCGGGAGAACGAGCAAAAGCGC |
| | ATCCTGGGCTTGCTGGAGAACTTCTAGAGGCACCTTGGCCCTGCGCATCATGGACTCT |
| | CTCAGCTTCCCTCCCAGGATCAG |

| | |
|---|---|
| ORF Start: ATG at 65 | ORF Stop: TAG at 431 |

| | | |
|---|---|---|
| SEQ ID NO: 262 | 122 aa | MW at 14385.4 kD |

| | |
|---|---|
| NOV91a, | MELHFKYLGAMQVADKKIEGEKHDMVRRGEIIDNDTEEEFYLRRLDAGLFVLQHICYI |
| CG59710-01 Protein Sequence | |
| | MAEICNANVPQIRQRVHQILNMRGSSIKIVRHIIKEYAENIGDGRSPEFRENEQKRIL |
| | GLLENF |

Further analysis of the NOV91a protein yielded the following properties shown in Table 91B.

TABLE 91B
Protein Sequence Properties NOV91a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | No Known Signal Sequence Predicted |
| analysis: | |

A search of the NOV91a protein against the Geneseq database, a proprietary database that contains sequences published in patents and patent publication, yielded several homologous proteins shown in Table 91C.

TABLE 91C
Geneseq Results for NOV91a

| | | NOV91a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU28058 | Novel human secretory protein, | 1 . . . 122 | 122/122 (100%) | 1e−66 |
| | Seq ID No 227-Homo sapiens, | 397 . . . 518 | 122/122 (100%) | |
| | 518 aa. [WO200166689-A2, | | | |
| | 13 SEP. 2001] | | | |
| AAM93729 | Human polypeptide, SEQ ID NO: | 1 . . . 122 | 122/122 (100%) | 1e−66 |
| | 3689-Homo sapiens, 563 aa. | 442 . . . 563 | 122/122 (100%) | |
| | [EP1130094-A2, 5 SEP. 2001] | | | |
| AAB63116 | Human secreted protein sequence | 1 . . . 119 | 119/119 (100%) | 1e−64 |
| | encoded by gene 39 SEQ ID | 283 . . . 401 | 119/119 (100%) | |
| | NO: 126 -Homo sapiens, 401 aa. | | | |
| | [WO200061748-A1, | | | |
| | 19 OCT. 2000] | | | |
| AAU28246 | Novel human secretory protein, | 1 . . . 118 | 104/120 (86%) | 2e−51 |
| | Seq ID No 603-Homo sapiens, | 197 . . . 316 | 106/120 (87%) | |
| | 360 aa. [WO200166689-A2, | | | |
| | 13 SEP. 2001] | | | |
| ABB21673 | Protein #3672 encoded by probe | 24 . . . 55 | 32/32 (100%) | 1e−11 |
| | for measuring heart cell gene | 1 . . . 132 | 32/32 (100%) | |
| | expression-Homo sapiens, 32 aa. | | | |
| | [WO200157274-A2, | | | |
| | 9 AUG. 2001] | | | |

In a BLAST search of public sequence databases, the NOV91a protein was found to have homology to the proteins shown in the BLASTP data in Table 91D.

TABLE 91D
Public BLASTP Results for NOV91a

| | | NOV91a | | |
|---|---|---|---|---|
| Protein | | Residues/ | Identities/ | |
| Accession | | Match | Similarities for | Expect |
| Numer | Protein/Organism/Length | Residues | the Matched Portion | Value |
| Q96KD2 | TESTES DEVELOPMENT- | 1 . . . 122 | 122/122 (100%) | 5e−66 |
| | RELATED NYD-SP19-Homo | 255 . . . 376 | 122/122 (100%) | |
| | sapiens (Human), 376 aa. | | | |
| Q9H7A5 | CDNA: FLJ21108 FIS, CLONE | 1 . . . 122 | 121/122 (99%) | 5e−65 |
| | CAS05257-Homo sapiens | 104 . . . 225 | 121/122 (99%) | |
| | (Human), 225 aa. | | | |
| O62703 | P14-Bos taurus (Bovine), 122 aa. | 1 . . . 122 | 116/122 (95%) | 2e−62 |
| | | 1 . . . 122 | 119/122 (97%) | |
| Q9CWL8 | 5730471K09RIK PROTEIN-Mus | 1 . . . 122 | 115/122 (94%) | 3e−62 |
| | musculus (Mouse), 563 aa. | 442 . . . 563 | 118/122 (96%) | |
| Q9Y3M7 | DJ633O20.1 (P14L, SIMILAR TO | 1 . . . 93 | 93/93 (100%) | 3e−48 |
| | BOS TAURUS P14)-Homo | 192 . . . 284 | 93/93 (100%) | |
| | sapiens (Human), 284 aa | | | |
| | (fragment). | | | |

PFam analysis predicts that the NOV91a protein contains the domains shown in the Table 91E.

TABLE 91E
Domain Analysis of NOV91a

| | | | |
|---|---|---|---|
| | | Identities/ | |
| Pfam | NOV91a | Similarities for | Expect |
| Domain | Match Region | the Matched Region | Value |

No Significant Matches Found

### Example 92

The NOV92 clone was analyzed, and the nucleotide and predicted polypeptide sequences are shown in Table 92A.

TABLE 92A
NOV92 Sequence Analysis

| | SEQ ID NO:263 | 6527 bp | |
|---|---|---|---|
| NOV92a, | CCACAGAGGGGAAATGCCAGCTTCCCTCTCCCTGGGGCTCCGTGCCCCCTCTGATCCA | | |
| CG59754-02 DNA Sequence | | | |
| | GCCCTTCGAATTCCCACCCGCCTCCATCGGCCAGCTGCTCTACATTCCCTGTGTGGTG | | |
| | TCCTCGGGGGACATGCCCATCCGTATCACCTGGAGGAAGGACGGACAGGTGATCATCT | | |
| | CAGGCTCGGGCGTGACCATCGAGAGCAAGGAATTCATGAGCTCCCTGCAGATCTCTAG | | |
| | CGTCTCCCTCAAGCACAACGGCAACTATACATGCATCGCCAGCAACGCAGCCGCCACC | | |
| | GTGAGCATTGTGTCTCCAGAACACAGGTTTTTTATTACCTACCACGGCGGGCTGTACA | | |
| | TCTCTGACGTACAGAAGGAGGACGCCCTCTCCACCTATCGCTGCATCACCAAGCACAA | | |
| | GTATAGCGGGGAGACCCGGCAGAGCAATGGGGCACGCCTCTCTGTGACAGACCCTGCT | | |
| | GAGTCGATCCCCACCATCCTGGATGGCTTCCACTCCCAGGAAGTGTGGGCCGGCCACA | | |
| | CCGTGGAGCTGCCCTGCACCGCCTCGGGCTACCCTATCCCCGCCATCCGCTGGCTCAA | | |
| | GGATGGCCGGCCCCTCCCGGCTGACAGCCGCTGGACCAAGCGCATCACAGGGCTGACC | | |
| | ATCAGCGACTTGCGGACCGAGGACAGCGGCACCTACATTTGTGAGGTCACCAACACCT | | |
| | TCGGTTCGGCAGAGGCCACAGGCATCCTCATGGTCATTGATCCCCTTCATGTGACCCT | | |
| | GACACCAAAGAAGCTGAAGACCGGCATTGGCAGCACGGTCATCCTCTCCTGTGCCCTG | | |
| | ACGGGCTCCCCAGAGTTCACCATCCGCTGGTATCGCAACACGGAGCTGGTGCTGCCTG | | |
| | ACGAGGCCATCTCCATCCGCGGGCTCAGCAACGAGACGCTGCTCATCACCTCGGCCCA | | |
| | GAAGAGCCATTCCGGGGCCTACCAGTGCTTCGCTACCCGCAAGGCCCAGACCGCCCAG | | |
| | GACTTTGCCATCATTGCACTTGAGGATGGCACGCCCCGCATCGTCTCGTCCTTCAGCG | | |
| | AGAAGGTGGTCAACCCCGGGGAGCAGTTCTCACTGATGTGTGCGGCCAAGGGCGCCCC | | |
| | GCCCCCCACGGTCACCTGGGCCCTCGACGATGAGCCCATCGTGCGGGATGGCAGCCAC | | |
| | CGCACCAACCAGTACACCATGTCGGACGGCACCACCATCAGCCACATGAACGTCACAG | | |
| | GCCCCCAGATCCGCGACGGGGGCGTGTACCGGTGCACAGCGCGGAACTTGGTGGGCAG | | |
| | TGCTGAATATCAGGCGCGAATAAACGTAAGAGGCCCACCCAGCATCCGGGCTATGCGG | | |
| | AACATCACAGCAGTCGCCGGGCGGGACACCCTTATCAACTGCAGGGTCATCGGCTATC | | |
| | CCTACTACTCCATCAAGTGGTACAAGGATGCCTTGCTGCTGCCAGACAACCACCGCCA | | |
| | GGTGGTGTTTGAGAATGGGACCCTCAAGCTGACTGACGTGCAGAAGGGCATGGATGAG | | |
| | GGGGAGTACCTGTGCAGTGTCCTCATCCAGCCCCAGCTCTCCATCAGCCAGAGCGTTC | | |
| | ACGTAGCCGTCAAAGTGCCCCCTCTGATCCAGCCCTTCGAATTCCCACCCGCCTCCAT | | |
| | CGGCCAGCTGCTCTACATTCCCTGTGTGGTGTCCTCGGGGGACATGCCCATCCGTATC | | |
| | ACCTGGAGGAAGGACGGACAGGTGATCATCTCAGGCTCGGGCGTGACCATCGAGAGCA | | |
| | AGGAATTCATGAGCTCCCTGCAGATCTCTAGCGTCTCCCTCAAGCACAACGGCAACTA | | |
| | TACATGCATCGCCAGCAACGCAGCCGCCACCGTGAGCCGGGAGCGTCAGCTCATCGTG | | |
| | CGTGTGCCCCCTCGATTTGTGGTGCAACCCAACAACCAGGATGGCATCTACGGCAAAG | | |
| | CTGGTGTGCTCAACTGCTCGGTGGACGGCTACCCCCCACCCAAGGTCATGTGGAAGCA | | |
| | TGCCAAGGGGAGCGGGAACCCCCAGCAGTACCACCCTGTGCCCCTCACTGGCCGCATC | | |
| | CAGATCCTGCCCAACAGCTCGCTGCTGATCCGCCACGTCCTAGAAGAGGACATCGGCT | | |
| | ACTACCTCTGCCAGGCCAGCAACGGCGTAGGCACCGACATCAGCAAGTCCATGTTCCT | | |
| | CACAGTCAAGATCCCGGCCATGATCACTTCCCACCCCAACACCACCATCGCCATCAAG | | |
| | GGCCATGCGAAGGAGCTAAACTGCACGGCACGGGGTGAGCGGCCCATCATCATCCGCT | | |
| | GGGAGAAGGGGGACACAGTCATCGACCCTGACCGCGTCATGCGGTATGCCATCGCCAC | | |
| | CAAGGACAACGGCGACGAGGTCGTCTCCACACTGAAGCTCAAGCCCGCTGACCGTGGG | | |
| | GACTCTGTGTTCTTCAGCTGCCATGCCATCAACTCGTATGGGGAGGACCGGGGCTTGA | | |
| | TCCAACTCACTGTGCAAGAGCCCCCCGACCCCCCAGAGCTGGAGATCCGGGAGGTGAA | | |
| | GGCCCGGAGCATGAACCTGCGCTGGACCCAGCGATTCGACGGGAACAGCATCATCACG | | |
| | GGCTTCGACATTGAATACAAGAACAAATCAGATTCCTGGGACTTCAAGCAGTCCACAC | | |
| | GCAACATCTCCCCCACCATCAACCAGGCCAACATTGTGGACTTGCACCCGGCATCTGT | | |
| | GTACAGCATCCGCATGTACTCTTTCAACAAGATTGGCCGCAGTGAACCAAGCAAGGAG | | |
| | CTCACCATCAGCACTGAGGAGGCCGCTCCCGATGGGCCCCCCATGGATGTTACCTTGC | | |
| | AGCCAGTGACCTCACAGAGCATCCAGGTGACCTGGAAGGCACCCAAGAAGGAGCTGCA | | |
| | GAACGGTGTCATCCGGGGCTACCAGATTGGCTACAGAGAGAACAGCCCCGGCAGCAAC | | |
| | GGGCAGTACAGCATCGTGGAGATGAAGGCCACGGGGGACAGCGAGGTCTACACCCTGG | | |
| | ACAACCTCAAGAAGTTCGCCCAGTATGGGGTGGTGGTCCAAGCCTTCAATCGGGCTGG | | |
| | CACGGGGCCCTCTTCCAGCGAGATCAATGCCACCACTCTGGAGGATGTGCCCAGCCAG | | |
| | CCCCCTGAGAACGTCCGGGCCCTGTCCATCACTTCTGACGTGGCCGTCATCTCCTGGT | | |
| | CAGAGCCCCCGCGCAGCACCCTCAATGGCGTCCTCAAAGGCTATCGGGTCATCTTCTG | | |
| | GTCCCTCTATGTTGATGGGGAGTGGGGCGAGATGCAGAACATCACCACCACGCGGGAG | | |
| | CGGGTGGAGCTGCGGGGCATGGAGAAGTTCACCAACTACAGCGTCCAGGTGCTGGCCT | | |
| | ACACCCAGGCTGGGGACGGCGTACGCAGCAGTGTGCTCTACATCCAGACCAAGGAGGA | | |
| | CGTTCCAGGTCCCCCTGCTGGCATCAAAGCTGTCCCTTCATCAGCTAGCAGTGTGGTT | | |
| | GTGTCTTGGCTCCCCCCTACCAAGCCCAACGGGGTGATCCGCAAGTACACCATCTTCT | | |
| | GTTCCAGCCCCGGGTCTGGCCAGCCGGCTCCCAGCGAGTACGAGACGAGTCCAGAGCA | | |
| | GCTCTTCTACCGGATCGCCCACCTAAACCGCGGTCAGCAGTATCTGCTGTGGGTGGCC | | |
| | GCCGTCACCTCTGCCGGCCGGGGCAACAGCAGCGAGAAGGTGACCATCGAGCCTGCTG | | |
| | GCAAGGCCCCAGCAAAGATCATCTCCTTTGGGGGCACCGTGACAACACCTTGGATGAA | | |
| | AGATGTTCGGCTGCCTTGCAATTCAGTGGGAGATCCAGCCCCTGCTGTGAAGTGGACC | | |
| | AAGGACAGTGAAGACTCGGCCATTCCAGTGTCCATGGATGGGCACCGGCTCATCCACA | | |
| | CCAATGGCACACTGCTGCTGCGTGCAGTGAAGGCTGAGGACTCTGGCTACTACACGTG | | |
| | CACGGCCACCAACACTGGTGGCTTTGACACCATCATCGTCAACCTTCTGGTGCAAGTT | | |
| | CCCCCGGACCAGCCCCGCCTCACTGTCTCCAAAACCTCAGCTTCGTCCATCACCCTGA | | |
| | CCTGGATTCCAGGTGACAATGGGGGCAGCTCCATCCGAGGCTTCGTGCTACAGTACTC | | |
| | GGTGGACAACAGCGAGGAGTGGAAGGATGTGTTCATCAGCTCCAGCGAGCGCTCCTTC | | |
| | AAGCTGGACAGCCTCAAGTGTGGCACGTGGTACAAGGTGAAGCTGGCAGCCAAGAACA | | |
| | GCGTGGGCTCTGGGCGCATCAGCGAGATCATCGAGGCCAAGACCCACGGGCGGGAGCC | | |
| | CTCCTTCAGCAAAGACCAACACCTCTTCACCCACATCAACTCCACGCATGCTCGGCTT | | |
| | AACCTGCAGGGCTGGAACAATGGGGGCTGCCCTATCACAGCCATCGTTCTGGAGTACC | | |
| | GGCCCAAGGGGACCTGGGCCTGGCAGGGCCTCCGGGCCAACAGCTCCGGGGAGGTGTT | | |
| | TCTGACGGAACTGCGAGAGGCCACGTGGTACGAGCTGCGCATGAGGGCTTGCAACAGT | | |
| | GCGGGCTGCGGCAATGAAACAGCCCAGTTCGCCACCCTGGACTACGATGGCAGCACCA | | |
| | TTCCACCCATCAAGTCTGCTCAAGGTGAAGGGGATGATGTGAAGAAGCTGTTCACCAT | | |
| | CGGCTGCCCTGTCATCCTGGCCACACTGGGGGTGGCACTGCTCTTCATCGTACGCAAG | | |
| | AAGAGGAAGGAGAAACGGCTGAAGCGACTCCGAGATGCAAAGAGTTTGGCAGAAATGT | | |
| | TGATAAGCAAGAACAATAGAAGCTTTGACACCCCTGTGAAAGGGCCACCCCAGGGCCC | | |
| | ACGGCTACACATTGACATCCCCAGGGTCCAGCTGCTCATCGAGGACAAAGAAGGCATC | | |
| | CTGTCAACCCACAGAGCTTCTGTACTGGCGTCTCCTTGCACCACCCAACCCTCATCCA | | |
| | GAGCACAGGACCCCTCATCGACATGTCTGACATCCGGCCAGGAACCAATCCAGTGTCC | | |
| | AGGAAGAATGTGAAGTCAGCCCACAGCACCCGGAACCGGTACTCAAGCCAGTGGACCC | | |
| | TGACCAAGTGCCAGGCCTCCACACCTGCCCGCACCCTCACCTCCGACTGGCGCACCGT | | |
| | GGGCTCCCAGCATGGTGTCACGGTCACTGAGAGTGACAGCTACAGTGCCAGCCTGTCC | | |
| | CAGGACACAGACAAAGGAAGGAACAGCATGGTGTCCACTGAGAGTGCCTCTTCCACCT | | |
| | ACGAGGAGCTGGCCCGGGCCTATGAGCATGCCAAGCTGGAGGAGCAGCTGCAGCACGC | | |
| | CAAGTTTGAGATCACCGAGTGCTTCATCTCTGACAGTTCCTCTGACCAGATGACCACA | | |
| | GGCACCAACGAGAACGCCGACAGCATGACATCCATGAGCACACCCTCAGAGCCTGGCA | | |
| | TCTGCCGCTTTACCGCCTCACCACCCAAGCCCCAGGATGCGGACCGGGGCAAAAACGT | | |
| | GGCTGTGCCCATCCCTCACCGGGCCAACAAGAGTGACTACTGCAACCTGCCCCTGTAT | | |
| | GCCAAGTCAGAGGCCTTCTTTCGAAAGGCAGATGGACGTGAGCCCTGCCCCGTGGTCC | | |
| | CACCCCGTGAGGCCTCCATCCGGAACCTGGCTCGAACCTACCACACCCAGGCTCGCCA | | |
| | CCTGACCCTGGACCCTGCCAGCAAGTCCTTGGGCCTTCCCCACCCAGGGGCCCCCGCT | | |
| | GCCGCCTCCACAGCCACCTTACCTCAGAGGACTCTGGCCATGCCAGCCCCCCCAGCCG | | |
| | GCACAGCCCCCCCAGCCCCCGGCCCCACCCCTGCTGAGCCACCCACCGCCCCCAGCGC | | |
| | TGCCCCTCCGGCCCCCAGCACCGAGCCTCCACGAGCCGGGGGCCCACACACCAAAATG | | |
| | GGGGGCTCCAGGGACTCGCTTCTCGAGATGAGCACATCGGGGGTAGGGAGGTCTCAGA | | |
| | AGCAGGGGGCCGGGGCCTACTCCAAATCCTACACCCTGGTGTAGGGCCGGCAGGAAGA | | |
| | GCAGCCACGCCTGGGCCGCGCCGCGCCGCAGCCCCACACGCCAGCTCGGCTGTTTTTC | | |
| | TGCATTATTTATATTCAACTGACAGACAAAAACCAACCAACGACAAAACAAAAACCCC | | |
| | CAATCATGAACGCCTGTACATAGAACTCTTTTGTACAAATGAAACTATTTTCTTCTTC | | |
| | TCCATGAAGCCAGGGCACAAAGAATTTGACAGTACAAGTCAAATCCCCCACCCCACAA | | |
| | AATATGTGTGGAGATATATATACATATATAGACAGACAGGAACGCCTCCACGAGCTAT | | |
| | ATATCTATATATTTCTCTCACCCTATTTTGAGACAGAGGCACAAAGACTCAGCAATTT | | |
| | TTTTCCCTCCTCCTCACCTTCCCCCCAGTCTAGGTGGTTTTGACAAAGACCAAAATCC | | |
| | CAACTCAGAGACACTGCATGCGATTTTACTGTTCCAAGAAAACCAGGAGTTGCTTCAA | | |
| | TTTGCAGATGCTTATGTGTTAATACCTTTTTCTATGAAAAAAGACCCAGCGCCGTGTG | | |
| | CAATAAAGGTTATGTTTCCAAAAAAAAGCTT | | |
| | ORF Start: ATG at 129 | | ORF Stop: TAG at 5958 |
| | SEQ ID NO:264 | 1943 aa | MW at 211904.3 kD |
| NOV92a, | MPIRITWRKDGQVIISGSGVTIESKEFMSSLQISSVSLKHNGNYTCIASNAAATVSIV | | |
| CG59754-02 Protein | | | |
| Sequence | SPEHRFFITYHGGLYISDVQKEDALSTYRCITKHKYSGETRQSNGARLSVTDPAESIP | | |
| | TILDGFHSQEVWAGHTVELPCTASGYPIPAIRWLKDGRPLPADSRWTKRITGLTISDL | | |
| | RTEDSGTYICEVTNTFGSAEATGILMVIDPLHVTLTPKKLKTGIGSTVILSCALTGSP | | |
| | EFTIRWYRNTELVLPDEAISIRGLSNETLLITSAQKSHSGAYQCFATRKAQTAQDFAI | | |
| | IALEDGTPRIVSSFSEKVVNPGEQFSLMCAAKGAPPPTVTWALDDEPIVRDGSHRTNQ | | |
| | YTMSDGTTISHMNVTGPQIRDGGVYRCTARNLVGSAEYQARINVRGPPSIRAMRNITA | | |
| | VAGRDTLINCRVIGYPYYSIKWYKDALLLPDNHRQVVFENGTLKLTDVQKGMDEGEYL | | |
| | CSVLIQPQLSISQSVHVAVKVPPLIQPFEFPPASIGQLLYIPCVVSSGDMPIRITWRK | | |
| | DGQVIISGSGVTIESKEFMSSLQISSVSLKHNGNYTCIASNAAATVSRERQLIVRVPP | | |
| | RFVVQPNNQDGIYGKAGVLNCSVDGYPPPKVMWKHAKGSGNPQQYHPVPLTGRIQILP | | |
| | NSSLLIRHVLEEDIGYYLCQASNGVGTDISKSMFLTVKIPAMITSHPNTTIAIKGHAK | | |
| | ELNCTARGERPIIIRWEKGDTVIDPDRVMRYAIATKDNGDEVVSTLKLKPADRGDSVF | | |
| | FSCHAINSYGEDRGLIQLTVQEPPDPPELEIREVKARSMNLRWTQRFDGNSIITGFDI | | |
| | EYKNKSDSWDFKQSTRNISPTINQANIVDLHPASVYSIRMYSFNKIGRSEPSKELTIS | | |
| | TEEAAPDGPPMDVTLQPVTSQSIQVTWKAPKKELQNGVIRGYQIGYRENSPGSNGQYS | | |
| | IVEMKATGDSEVYTLDNLKKFAQYGVVVQAFNRAGTGPSSSEINAEELEDVPSQPPEN | | |
| | VRALSITSDVAVISWSEPPRSTLNGVLKGYRVIFWSLYVDGEWGEMQNITTTRERVEL | | |
| | RGMEKFTNYSVQVLAYTQAGDGVRSSVLYIQTKEDVPGPPAGIKAVPSSASSVVVSWL | | |
| | PPTKPNGVIRKYTIFCSSPGSGQPAPSEYETSPEQLFYRIAHLNRGQQYLLWVAAVTS | | |
| | AGRGNSSEKVTIEPAGKAPAKIISFGGTVTTPWMKDVRLPCNSVGDPAPAVKWTKDSE | | |
| | DSAIPVSMDGHRLIHTNGTLLLRAVKAEDSGYYTCTATNTGGFDTIIVNLLVQVPPDQ | | |
| | PRLTVSKTSASSITLTWIPGDNGGSSIRGFVLQYSVDNSEEWKDVFISSSERSFKLDS | | |
| | LKCGTWYKVKLAAKNSVGSGRISEIIEAKTHGREPSFSKDQHLFTHINSTHARLNLQG | | |
| | WNNGGCPITAIVLEYRPKGTWAWQGLRANSSGEVFLTELREATWYELRMRACNSAGCG | | |
| | NETAQFATLDYDGSTIPPIKSAQGEGDDVKKLFTIGCPVILATLGVALLFIVRKKRKE | | |
| | KRLKRLRDAKSLAEMLISKNNRSFDTPVKGPPQGPRLHIDIPRVQLLIEDKEGIKQLG | | |
| | DDKATIPVTDAEFSQAVNPQSFCTGVSLHHPTLIQSTGPLIDMSDIRPGTNPVSRKNV | | |
| | KSAHSTRNRYSSQWTLTKCQASTPARTLTSDWRTVGSQHGVTVTESDSYSASLSQDTD | | |
| | KGRNSMVSTESASSTYEELARAYEHAKLEEQLQHAKFEITECFISDSSSDQMTTGTNE | | |
| | NADSMTSMSTPSEPGICRFTASPPKPQDADRGKNVAVPIPHRANKSDYCNLPLYAKSE | | |
| | AFFRKADGREPCPVVPPREASIRNLARTYHTQARHLTLDPASKSLGLPHPGAPAAAST | | |
| | DSLLEMSTSGVGRSQKQGAGAYSKSYTLV | | |
| | SEQ ID NO:265 | 6049 bp | |
| NOV92b, | CCACAGAGGGGAAATGCCAGCTTCCCTCTCCCTGGGGCTCCGTGCCCCCTCTGATCCA | | |
| CG59754-01 DNA Sequence | | | |
| | GCCCTTCGAATTCCCACCCGCCTCCATCGGCCAGCTGCTCTACATTCCCTGTGTGGTG | | |
| | TCCTCGGGGGACATGCCCATCCGTATCACCTGGAGGAAGGACGGACAGGTGATCATCT | | |
| | CAGGCTCGGGCGTGACCATCGAGAGCAAGGAATTCATGAGCTCCCTGCAGATCTCTAG | | |
| | CGTCTCCCTCAAGCACAACGGCAACTATACATGCATCGCCAGCAACGCAGCCGCCACC | | |
| | GTGAGCATTGTGTCTCCAGAACACAGGTTTTTTATTACCTACCACGGCGGGCTGTACA | | |
| | TCTCTGACGTACAGAAGGAGGACGCCCTCTCCACCTATCGCTGCATCACCAAGCACAA | | |
| | GTATAGCGGGGAGACCCGGCAGAGCAATGGGGCACGCCTCTCTGTGACAGACCCTGCT | | |
| | GAGTCGATCCCCACCATCCTGGATGGCTTCCACTCCCAGGAAGTGTGGGCCGGCCACA | | |
| | CCGTGGAGCTGCCCTGCACCGCCTCGGGCTACCCTATCCCCGCCATCCGCTGGCTCAA | | |
| | GGATGGCCGGCCCCTCCCGGCTGACAGCCGCTGGACCAAGCGCATCACAGGGCTGACC | | |
| | ATCAGCGACTTGCGGACCGAGGACAGCGGCACCTACATTTGTGAGGTCACCAACACCT | | |
| | TCGGTTCGGCAGAGGCCACAGGCATCCTCATGGTCATTGATCCCCTTCATGTGACCCT | | |
| | GACACCAAAGAAGCTGAAGACCGGCATTGGCAGCACGGTCATCCTCTCCTGTGCCCTG | | |
| | ACGGGCTCCCCAGAGTTCACCATCCGCTGGTATCGCAACACGGAGCTGGTGCTGCCTG | | |
| | ACGAGGCCATCTCCATCCGCGGGCTCAGCAACGAGACGCTGCTCATCACCTCGGCCCA | | |
| | GAAGAGCCATTCCGGGGCCTACCAGTGCTTCGCTACCCGCAAGGCCCAGACCGCCCAG | | |
| | GACTTTGCCATCATTGCACTTGAGGATGGCACGCCCCGCATCGTCTCGTCCTTCAGCG | | |
| | AGAAGGTGGTCAACCCCGGGGAGCAGTTCTCACTGATGTGTGCGGCCAAGGGCGCCCC | | |
| | GCCCCCCACAGTCACCTGGGCCCTCGACGATGAGCCCATCGTGCGGGATGGCAGCCAC | | |
| | CGCACCAACCAGTACACCATGTCGGACGGCACCACCATCAGCCACATGAACGTCACAG | | |
| | GCCCCCAGATCCGCGACGGGGGCGTGTACCGGTGCACAGCGCGGAACTTGGTGGGCAG | | |
| | TGCTGAATATCAGGCGCGAATAAACGTAAGAGGCCCACCCAGCATCCGGGCTATGCGG | | |
| | AACATCACAGCAGTCGCCGGGCGGGACACCCTTATCAACTGCAGGGTCATCGGCTATC | | |
| | CCTACTACTCCATCAAGTGGTACAAGGATGCCTTGCTGCTGCCAGACAACCACCGCCA | | |
| | GGTGGTGTTTGAGAATGGGACCCTCAAGCTGACTGACGTGCAGAAGGGCAGGGATGAG | | |
| | GGGGAGTACCTGTGCAGTGTCCTCATCCAGCCCCAGCTCTCCATCAGCCAGAGCGTTC | | |
| | ACGTAGCCGTCAAAGTGCCCCCTCTGATCCAGCCCTTCGAATTCCCACCCGCCTCCAT | | |
| | CGGCCAGCTGCTCTACATTCCCTGTGTGGTGTCCTCGGGGGACATGCCCATCCGTATC | | |
| | ACCTGGAGGAAGGACGGACAGGTGATCATCTCAGGCTCGGGCGTGACCATCGAGAGCA | | |
| | AGGAATTCATGAGCTCCCTGCAGATCTCTAGCGTCTCCCTCAAGCACAACGGCAACTA | | |
| | TACATGCATCGCCAGCAACGCAGCCGCCACCGTGAGCCGGGAGCGTCAGCTCATCGTG | | |
| | CGTGTGCCCCCTCGATTTGTGGTGCAACCCAACAACCAGGATGGCATCTACGGCAAAG | | |
| | CTGGTGTGCTCAACTGCTCGGTGGACGGCTACCCCCCACCCAAGGTCATGTGGAAGCA | | |
| | TGCCAAGGGTAGCGGGAACCCCCAGCAGTACCACCCTGTGCCCCTCACTGGCCGCATC | | |
| | CAGATCCTGCCCAACAGCTCGCTGCTGATCCGCCACGTCCTAGAAGAGGACATCGGCT | | |
| | ACTACCTCTGCCAGGCCAGCAACGGCGTAGGCACCGACATCAGCAAGTCCATGTTCCT | | |
| | CACAGTCAAGATCCCCACCATCCTGGATGGCTTCCACTCCCAGGAAGTGTGGGCCGGC | | |
| | CACACCGTGGAGCTGCCCTGCACCGCCTCGGGCTACCCTATCCCCGCCATCCGCTGGC | | |
| | TCAAGGATGGCCGGCCCCTCCCGGCTGACAGCCGCTGGACCAAGCGCATCACAGGGCT | | |
| | GACCATCAGCGACTTGCGGACCGAGGACAGCGGCACCTACATTTGTGAGGTCACCAAC | | |
| | ACCTTCGGTGAGGCCACAGGCATCCTCATGGTCATTGGTGAGGAGCCCCCCGACCCCC | | |
| | CAGAGCTGGAGATCCGGGAGGTGAAGGCCCGGAGCATGAACCTGCGCTGGACCCAGCG | | |
| | ATTCGACGGGAACAGCATCATCACGGGCTTCGACATTGAATACAAGAACAAATCAGAT | | |
| | TCCTGGGACTTCAAGCAGTCCACACGCAACATCTCCCCCACCATCAACCAGGCCAACA | | |
| | TTGTGGACTTGCACCCGGCATCTGTGTACAGCATCCGCATGTACTCTTTCAACAAGAT | | |
| | TGGCCGCAGTGAACCAAGCAAGGAGCTCACCATCAGCACTGAGGAGGCCTCAGCTCCC | | |
| | GATGGGCCCCCCATGGATGTTACCTTGCAGCCAGTGACCTCACAGAGCATCCAGGTGA | | |
| | CCTGGAAGCAGGCACCCAAGAAGGAGCTGCAGAACGGTGTCATCCGGGGCTACCAGAT | | |
| | TGGCTACAGAGAGAACAGCCCCGGCAGCAACGGGCAGTACAGCATCGTGGAGATGAAG | | |
| | GCCACGGGGGACAGCGAGGTCTACACCCTGGACAACCTCAAGAAGTTCGCCCAGTATG | | |
| | GGGTGGTGGTCCAGGCCTTCAATCGGGCTGGCACGGGGCCCTCTTCCAGCGAGATCAA | | |
| | TGCCACCACTCTGGAGGATGTGCCCAGCCAGCCCCCTGAGAACGTCCGGGCCCTGTCC | | |
| | ATCACTTCTGACGTGGCCGTCATCTCCTGGTCAGAGCCCCCGCGCAGCACCCTCAATG | | |
| | GCGTCCTCAAAGGCTATCGGGTCATCTTCTGGTCCCTCTATGTTGATGGGGAGTGGGG | | |
| | CGAGATGCAGAACATCACCACCACGCGGGAGCGGGTGGAGCTGCGGGGCATGGAGAAG | | |
| | TTCACCAACTACAGCGTCCAGGTGCTGGCCTACACCCAGGCTGGGGACGGCGTACGCA | | |
| | GCAGTGTGCTCTACATCCAGACCAAGGAGGACGTTCCAGGTCCCCCTGCTGGCATCAA | | |
| | AGCTGTCCCTTCATCAGCTAGCAGTGTGGTTGTGTCTTGGCTCCCCCCTACCAAGCCC | | |
| | AACGGGGTGATCCGCAAGTACACCATCTTCTGTTCCAGCCCCGCCCCGCAGGCTCCCA | | |
| | GCGAGTACGAGACGAGTCCAGAGCAGCTCTTCTACCGGATCGCCCACCTAAACCGCGG | | |
| | TCAGCAGTATCTGCTGTGGGTGGCCGCCGTCACCTCTGCCGGCCGGGGCAACAGCAGC | | |
| | GAGAAGGTGACCATCGAGCCTGCTGGCAAGGCCCCAGCAAAGATCATCTCCTTTGGGG | | |
| | GCACCGTGACAACACCTTGGATGAAAGATGTTCGGCTGCCTTGCAATTCAGTGGGAGA | | |
| | TCCAGCCCCTGCTGTGAAGTGGACCAAGGACAGTGAAGACTCGGCCATTCCAGTGTCC | | |
| | ATGGATGGGCACCGGCTCATCCACACCAATGGCACACTGCTGCTGCGTGCAGTGAAGG | | |
| | CTGAGGACTCTGGCTACTACACGTGCACGGCCACCAACACTGGTGGCTTTGACACCAT | | |
| | CATCGTCAACCTTCTGGTGCAAGTTCCCCCGGACCAGCCCCGCCTCACTGTCTCCAAA | | |
| | ACCTCAGCTTCGTCCATCACCCTGACCTGGATTCCAGGTGACAATGGGGGCAGCTCCA | | |
| | TCCGAGGTTTTGTGCTACAGTACTCGGTGGACAACAGCGAGGAGTGGAAGGATGTGTT | | |
| | CATCAGCTCCAGCGAGCGCTCCTTCAAGCTGGACAGCCTCAAGTGTGGCACGTGGTAC | | |
| | AAGGTGAAGCTGGCAGCCAAGAACAGCGTGGGCTCTGGGCGCATCAGCGAGATCATCG | | |
| | AGGCCAAGACCCACGGGCGGGAGCCCTCCTTCAGCAAAGACCAACACCTCTTCACCCA | | |
| | CATCAACTCCACGCATGCTCGGCTTAACCTGCAGGGCTGGAACAATGGGGGCTGCCCT | | |
| | ATCACAGCCATCGTTCTGGAGTACCGGCCCAAGGGGACCTGGGCCTGGCAGGGCCTCC | | |
| | GGGCCAACAGCTCCGGGGAGGTGTTTCTGACGGAACTGCGAGAGGCCACGTGGTACGA | | |
| | GCTGCGCATGAGGGCTTGCAACAGTGCGGGCTGCGGCAATGAAACAGCCCAGTTCGCC | | |
| | ACCCTGGACTACGATGGCAGTACCATTCCACCCATCAAGTCTGCTCAAGGTGAAGGGG | | |
| | ATGATGTGAAGAAGCTGTTCACCATCGGCTGCCCTGTCATCCTGGCCACACTGGGGGT | | |
| | GGCACTGCTCTTCATCGTACGCAAGAAGAGGAAGGAGAAACGGCTGAAGCGACTCCGA | | |
| | GATGCAAAGAGTTTGGCAGAAATGTTGATAAGCAAGAACAATAGAAGCTTTGACACCC | | |
| | CTGTGAAAGGGCCACCCCAGGGCCCACGGCTACACATTGACATCCCCAGGGTCCAGCT | | |
| | GCTCATCGAGGACAAAGAAGGCATCAAGCAACTGGGTGAGGACAAGGCCACCATCCCT | | |
| | GTGACAGATGCTGAGTTCAGCCAAGCTGTCAACCCACAGAGCTTCTGTACTGGCGTCT | | |
| | CCTTGCACCACCCAACCCTCATCCAGAGCACAGGACCCCTCATCGACATGTCTGACAT | | |
| | CCGGCCAGGAACCGATCCAGTGTCCAGGAAGAATGTGAAGTCAGCCCACAGCACCCGG | | |
| | AACCGGTACTCAAGCCAGTGGACCCTGACCAAGTGCCAGGCCTCCACACCTGCCCGCA | | |
| | CCCTCACCTCCGACTGGCGCACCGTGGGCTCCCAGCATGGTGTCACGGTCACTGAGAG | | |
| | TGACAGCTACAGTGCCAGCCTGTCCCAGGACACAGACAAAGGAAGGAACAGCATGGTG | | |
| | TCCACTGAGAGTGCCTCTTCCACCTACGAGGAGCTGGCCCGGGCCTATGAGCATGCCA | | |
| | AGCTGGAGGAGCAGCTGCAGCACGCCAAGTTTGAGATCACCGAGTGCTTCATCTCTGA | | |
| | CAGTTCCTCTGACCAGATGACCACAGGCACCAACGAGAACGCCGACAGCATGACATCC | | |
| | ATGAGCACACCCTCAGAGCCTGGCATCTGCCGCTTTACCGCCTCACCACCCAAGCCCC | | |
| | AGGATGCGGACCGGCTGCTGATGCTGGTCCCAGGTGCCCACCTCCCTCCTCAGTCCAT | | |
| | CCATGTTGTAGCATATGTCAGAATTTCCTTCTTACTGAACAAGGGTGGGGGAGACCTG | | |
| | GCTTCTGATCTTAGCTCCGGCAGAGCTTGCAGTGAGCCGAGATCACGCGGCACCCGGC | | |
| | CACCAACACTGGTGGCTTTGACACCATCATCGTCAACCTGTGAGGCAGGTGACCCCAG | | |
| | GTGGGGACAGGGATGGAGAAAGGGTAGGGATTCCATCATGCGAGAGGGTCATCGAATG | | |
| | GAAGAAGCCAAACCAAGGGAGAGACAGACCTCTGGAGAAACAGAGGTGCACATGGAAG | | |
| | GGGAGGCAGGAGAGCTGGGGAGTGGGAGTGGGAGTGAGGGTGTGGGAGAGCCAGCACC | | |
| | TTCCCGTCACGGGGGGACTCCCCACACCCCATCACAGGGTCCGCCCTTGTGCTAAGGG | | |
| | GTGGTGGCTTTCCCCTCACAGTTCCCCCGGACCAGCCCCGCCTCACTGTCTCCAAAAC | | |
| | CTCAGCTTCGTCCATCACCCTGACCTGGATTCCAGGTGACAATGGGGGCAGCTCCATC | | |
| | CGAGGTGAGGAGGGGTCTGGATGCGGGGGAAGATAGGGGAAGGAATTCTGGGCCCGGG | | |
| | GCAGGGAAGGGGCTTCA | | |
| | ORF Start: ATG at 129 | | ORF Stop: TAA at 5853 |
| | SEQ ID NO:266 | 1908 aa | MW at 208575.3 kD |

| | |
|---|---|
| NOV92b, | MPIRITWRKDGQVIISGSGVTIESKEFMSSLQISSVSLKHNGNYTCIASNAAATVSIV |
| CG59754-01 Protein | |
| Sequence | SPEHRFFITYHGGLYISDVQKEDALSTYRCITKHKYSGETRQSNGARLSVTDPAESIP |
| | TILDGFHSQEVWAGHTVELPCTASGYPIPAIRWLKDGRPLPADSRWTKRITGLTISDL |
| | RTEDSGTYICEVTNTFGSAEATGILMVIDPLHVTLTPKKLKTGIGSTVILSCALTGSP |
| | EFTIRWYRNTELVLPDEAISIRGLSNETLLITSAQKSHSGAYQCFATRKAQTAQDFAI |
| | IALEDGTPRIVSSFSEKVVNPGEQFSLMCAAKGAPPPTVTWALDDEPIVRDGSHRTNQ |
| | YTMSDGTTISHMNVTGPQIRDGGVYRCTARNLVGSAEYQARINVRGPPSIRAMRNITA |
| | VAGRDTLINCRVIGYPYYSIKWYKDALLLPDNHRQVVFENGTLKLTDVQKGMDEGEYL |
| | CSVLIQPQLSISQSVHVAVKVPPLIQPFEFPPASIGQLLYIPCVVSSGDMPIRITWRK |
| | DGQVIISGSGVTIESKEFMSSLQISSVSLKHNGNYTCIASNAAATVSRERQLIVRVPP |
| | RFVVQPNNQDGIYGKAGVLNCSVDGYPPPKVMWKHAKGSGNPQQYHPVPLTGRIQILP |
| | NSSLLIRHVLEEDIGYYLCQASNGVGTDISKSMFLTVKIPTILDGFHSQEVWAGHTVE |
| | LPCTASGYPIPAIRWLKDGRPLPADSRWTKRITGLTISDLRTEDSGTYICEVTNTFGE |
| | ATGILMVIGEEPPDPPELEIREVKARSMNLRWTQRFDGNSIITGFDIEYKNKSDSWDF |
| | KQSTRNISPTINQANIVDLHPASVYSIRMYSFNKIGRSEPSKELTISTEEASAPDGPP |
| | MDVTLQPVTSQSIQVTWKQAPKKELQNGVIRGYQIGYRENSPGSNGQYSIVEMKATGD |
| | SEVYTLDNLKKFAQYGVVVQAFNRAGTGPSSSEINATTLEDVPSQPPENVRALSITSD |
| | VAVISWSEPPRSTLNGVLKGYRVIFWSLYVDGEWGEMQNITTTRERVELRGMEKFTNY |
| | SVQVLAYTQAGDGVRSSVLYIQTKEDVPGPPAGIKAVPSSASSVVVSWLPPTKPNGVI |
| | RKYTIFCSSPAPQAPSEYETSPEQLFYRIAHLNRGQQYLLWVAAVTSAGRGNSSEKVT |
| | IEPAGKAPAKIISFGGTFTTPWMKDVRLPCNSVGDPAPAVKWTKDSEDSAIPVSMDGH |
| | RLIHTNGTLLLRAVKAEDSGYYTCTATNTGGFDTIIVNLLVQVPPDQPRLTVSKTSAS |
| | SITLTWIPGDNGGSSIRGFVLQYSVDNSEEWKDVFISSSERSFKLDSLKCGTWYKVKL |
| | AAKNSVGSGRISEIIEAKTHGREPSFSKDQHLFTHINSTHARLNLQGWNNGGCPITAI |
| | VLEYRPKGTWAWQGLRANSSGEVFLTELREATWYELRMRACNSAGCGNETAQFATLDY |
| | DGSTIPPIKSAQGEGDDVKKLFTIGCPVILATLGVALLFIVRKKRKEKRLKRLRDAKS |
| | LAEMLISKNNRSFDTPVKGPPQGPRLHIDIPRVQLLIEDKEGIKQLGEDKATIPVTDA |
| | EFSQAVNPQSFCTGVSLHHPTLIQSTGPLIDMSDIRPGTDPVSRKNVKSAHSTRNRYS |
| | SQWTLTKCQASTPARTLTSDWRTVGSQHGVTVTESDSYSASLSQDTDKGRNSMVSTES |
| | ASSTYEELARAYEHAKLEEQLQHAKFEITECFISDSSSDQMTTGTNENADSMTSMSTP |
| | SEPGICRFTASPPKPQDADRLLMLVPGAHLPPQSIHVVAYVRISFLLNKGGGDLASDL |
| | SSGRACSEPRSRGTRPPTLVALTPSSSTCEAGDPRWGQGWRKGRDSIMREGHRMEEAK |
| | PRERQTSGETEVHMEGEAGELGSGSGSEGVGEPAPSRHGGTPHTPSQGPPLC |

Sequence comparison of the above protein sequences yields the following sequence relationships shown in Table 92B.

TABLE 92B
Comparison of NOV92a against NOV92b.

| | | Identities/ |
|---|---|---|
| Protein | NOV92a Residues/ | Similarities for |
| Sequence | Match Residues | the Matched Region |
| NOV92b | 1 . . . 1771 | 1663/1773 (93%) |
| | 1 . . . 1760 | 1681/1773 (94%) |

Further analysis of the NOV92a protein yielded the following properties shown in Table 92C.

TABLE 92C
Protein Sequence Properties NOV92a

| | |
|---|---|
| PSort | 0.7000 probability located in plasma membrane; 0.3000 |
| analysis: | probability located in microbody (peroxisome); 0.3000 |
| | probability located in nucleus; 0.2000 probability located |
| | in endoplasmic reticulum (membrane) |
| SignalP | No Known Signal Sequence Predicted |
| analysis: | |

A search of the NOV92a protein against the Geneseq database, a proprietary database that contains sequences published in patents and patent publication, yielded several homologous proteins shown in Table 92D.

TABLE 92D
Geneseq Results for NOV92a

| | | NOV92a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU28091 | Novel human secretory protein, | 200 . . . 1943 | 1744/1744 (100%) | 0.0 |
| | Seq ID No 260-Homo sapiens, | 1 . . . 1744 | 1744/1744 (100%) | |
| | 1744 aa. [WO200166689-A2, | | | |
| | 13 SEP. 2001] | | | |
| AAM78713 | Human protein SEQ ID NO 1375- | 200 . . . 1943 | 1744/1744 (100%) | 0.0 |
| | Homo sapiens, 1744 aa. | 1 . . . 1744 | 1744/1744 (100%) | |
| | [WO200157190-A2, | | | |
| | 9 AUG. 2001] | | | |
| AAM39040 | Human polypeptide SEQ ID NO | 200 . . . 1943 | 1744/1744 (100%) | 0.0 |
| | 2185-Homo sapiens, 1744 aa. | 1 . . . 1744 | 1744/1744 (100%) | |
| | [WO200153312-A1, | | | |
| | 26 JUL. 2001] | | | |
| AAW42086 | Human Down syndrome-cell | 44 . . . 1778 | 1085/1745 (62%) | 0.0 |
| | adhesion molecule DS-CAM1- | 154 . . . 1890 | 1357/1745 (77%) | |
| | [WO9817795-A1, 30 APR. 1998] | | | |
| AAW42087 | Human Down syndrome-cell | 44 . . . 1457 | 890/1416 (62%) | 0.0 |
| | adhesion molecule DS-CAM2- | 154 . . . 1564 | 1109/1416 (77%) | |
| | Homo sapiens, 1571 aa. | | | |
| | [WO9817795-A1, 30 APR. 1998] | | | |

In a BLAST search of public sequence databases, the NOV92a protein was found to have homology to the proteins shown in the BLASTP data in Table 92E.

TABLE 92E
Public BLASTP Results for NOV92a

| | | NOV92a | | |
|---|---|---|---|---|
| Protein | | Residues/ | Identities/ | |
| Accession | | Match | Similarities for | Expect |
| Number | Protein/Organism/Length | Residues | the Matched Portion | Value |
| AAL57166 | DOWN SYNDROME CELL | 44 . . . 1943 | 1889/1900 (99%) | 0.0 |
| | ADHESION MOLECULE | 155 . . . 2053 | 1892/1900 (99%) | |
| | DSCAML1-Homo sapiens | | | |
| | (Human), 2053 aa. | | | |
| Q9ULT7 | KIAA1132 PROTEIN-Homo | 122 . . . 1943 | 1822/1822 (100%) | 0.0 |
| | sapiens (Human), 1822 aa | 1 . . . 1822 | 1822/1822 (100%) | |
| | (fragment). | | | |
| O60469 | Down syndrome cell adhesion | 44 . . . 1943 | 1123/1920 (58%) | 0.0 |
| | molecule precursor (CHD2)- | 154 . . . 2012 | 1410/1920 (72%) | |
| | Homo sapiens (Human), | | | |
| | 2012 aa. | | | |
| Q9ERC8 | DOWN SYNDROME CELL | 44 . . . 1943 | 1119/1921 (58%) | 0.0 |
| | ADHESION MOLECULE- | 154 . . . 2013 | 1405/1921 (72%) | |
| | Mus musculus (Mouse), | | | |
| | 2013 aa. | | | |
| AAL57167 | DOWN SYNDROME CELL | 44 . . . 1943 | 1119/1921 (58%) | 0.0 |
| | ADHESION MOLECULE | 154 . . . 2013 | 1405/1921 (72%) | |
| | DSCAM-Rattus norvegicus | | | |
| | (Rat), 2013 aa. | | | |

PFam analysis predicts that the NOV92a protein contains the domains shown in the Table 92F.

TABLE 92F
Domain Analysis of NOV92a

| | | Identities/ | |
|---|---|---|---|
| Pfam | NOV92a | Similarities for | Expect |
| Domain | Match Region | the Matched Region | Value |
| ig: domain 1 of 10 | 1 . . . 48 | 12/49 (24%) | 2.7e−05 |
| | | 38/49 (78%) | |
| ig: domain 2 of 10 | 72 . . . 90 | 8/19 (42%) | 85 |
| | | 14/19 (74%) | |
| ig: domain 3 of 10 | 130 . . . 186 | 22/60 (37%) | 2.1e−14 |
| | | 46/60 (77%) | |
| ig: domain 4 of 10 | 219 . . . 278 | 16/63 (25%) | 4.9e−09 |
| | | 44/63 (70%) | |
| ig: domain 5 of 10 | 312 . . . 377 | 14/69 (20%) | 1.5e−07 |
| | | 50/69 (72%) | |
| ig: domain 6 of 10 | 409 . . . 467 | 12/61 (20%) | 4.8e−05 |
| | | 41/61 (67%) | |
| ig: domain 7 of 10 | 500 . . . 561 | 17/64 (27%) | 3.2e−11 |
| | | 49/64 (77%) | |
| ig: domain 8 of 10 | 594 . . . 659 | 19/69 (28%) | 9.4e−07 |
| | | 47/69 (68%) | |
| ig: domain 9 of 10 | 693 . . . 759 | 9/70 (13%) | 7.9e−06 |
| | | 47/70 (67%) | |
| fn3: domain 1 of 6 | 777 . . . 864 | 22/89 (25%) | 3e−16 |
| | | 65/89 (73%) | |
| fn3: domain 2 of 6 | 876 . . . 968 | 33/93 (35%) | 3.1e−16 |
| | | 68/93 (73%) | |
| fn3: domain 3 of 6 | 980 . . . 1069 | 26/93 (28%) | 2.9e−16 |
| | | 69/93 (74%) | |
| fn3: domain 4 of 6 | 1081 . . . 1167 | 24/88 (27%) | 3.7e−17 |
| | | 64/88 (73%) | |
| ig: domain 10 of 10 | 1194 . . . 1255 | 17/65 (26%) | 4.3e−09 |
| | | 46/65 (71%) | |
| fn3: domain 5 of 6 | 1274 . . . 1357 | 30/86 (35%) | 1.2e−18 |
| | | 67/86 (78%) | |
| fn3: domain 6 of 6 | 1371 . . . 1453 | 27/86 (31%) | 0.045 |
| | | 53/86 (62%) | |

### Example 93

The NOV93 clone was analyzed, and the nucleotide and predicted polypeptide sequences are shown in Table 93A.

TABLE 93A
NOV93 Sequence Analysis

| | SEQ ID NO:267 | 1272 bp | |
|---|---|---|---|
| NOV93a, | AGAGCCTGGTATGCAGGAGGTGCTCTGTAAATACCTGCCCCATACATACCCGCCCCAT | | |
| CG59800-01 DNA Sequence | | | |
| | ACATACCCACCCCATACATACCCACCCCATACATACCTGCCCTGTCCATACCTCCCCC | | |
| | CTACATACCTGCCCCGTCCATACCTGCCCCCTACATACCTGCCCCGTCCATACCTGCC | | |
| | CCCTACATACCTGCTCTGTCTATACCTGTGGCTAGGACTGTGGCCTTCCTTCTTAGCC | | |
| | GCTCAGAGCCTGCCTCCTCCTCTGCAGAGTGGCGGTGGTAGCAGGGCTTCCCGCGCGC | | |
| | CGATGCTGCTCGTGGCCCTGGTGCTCGGCGCCTACTGCCTCTGCGCCCTCCCCGGCCG | | |
| | CTGCCCGCCCGCCGCCCCCGCCCCCGCGCCCGCCCCCGCGCCCTCCGAGCCGTCCAGC | | |
| | TCCGTCCACCGCCCGGGACCACCCGGCCTGCCTTTGGCCACCGGTCCCGGCCGCCGGC | | |
| | CCTTCCCGCAACCGCTCATCGTTGGCGTGAAGAAGGGCGGCACGCGCGCCCTGCTGGA | | |
| | GTTTCTCCGGCTGCACCCCGACCTCCGCGCGCTCGCCTCTGAGCCCCACTTCTTCGAC | | |
| | AGGTGCCCCGACCGCGGCCTCCCCTGGTCCCGCAGTCTGATGCCCCGAACCCTCCATG | | |
| | GGCAGATCACCATGGAGACGACCCCGGGCTACTTCGTGACGCGAGAGGCCCCCCGCCG | | |
| | CATCCACGCCATGTCCCCGGACACGAAGCTGATCGTGGTGGTGCGGAACCCCGTCACC | | |
| | CGGGCCATCTCCGACTAGGCCCACACGCTCTCCAAGACCCCGGGCCTGCCCAGCTTCC | | |
| | GCGCCCTGGCCTTCCGCCACGGCCTGGGCCCCGTGGACACAGCCTGGAGCGCCGTCCG | | |
| | CATCCGCCTGTACGCCCACCACCTGGACCACTGGCTGCGCTACTTCCCCCTGTCCCAC | | |
| | TTCCTGTTCGTCAGCGGGGAGCGTCTGGTCAGCGACCCGGCCGGAGAGCTCGGCCGCG | | |
| | TGCAGGACTTCCTGGGCCTGAAACGGGTCGTCACGGACAAGCACTTCTACTTCAACGC | | |
| | CACCAAGGGCTTCCCCTGCCTCAAGAAGGCCCAGGGCGGCAGCCCTCCCCCCTGCCTG | | |
| | GGCAAGTCCAAGGGCCGGCCACACCCACCCGTGCCCCAGGCCGTGGTCCGGCGCCTGC | | |
| | AGGAGTTCTACCCGCCCTTCAACCGCAGGTTCTACCAGATCACGGGCCAGGACTTCGG | | |
| | CTGGGGCTGAGCGGCACCCTGGGCATGCTCAGCACCTTGATTGACACCCGCTCG | | |
| | ORF Start: GAG at 2 | | ORF Stop: GGC at 1217 |
| | SEQ ID NO:268 | 405 aa | MW at 43994.8 kD |

| | |
|---|---|
| NOV93a | MQEVLCKYLPHTYPPHTYPPHTYPPHTYLPCPYLPPTYLPRPYLPPTYLPRPYLPPTY |
| CG59800-01 Protein Sequence | |
| | LLCLYLWLGLWFCFIQSLFPPLQSGGGSRASRAPMLLVALVLGAYCLCALPGRCPF |
| | AARAPAPAPAPSEPSSSVERPGAPGLPLASGPGRRRFPQALIVGVKKGGTRALLEFLR |
| | LHPDXRALGSEXEFFDRCXXXCLXWXRSLMPRTLDGQTTMEXTPXYFVTREAPRRIHA |
| | MSPDTKLIVVVRNPVTRAISDXXQTLSKTPGLPSFRALAFRHGLGPVDTAWSAVRIGL |
| | YAQHLDEWLRYFPLSEELFVSGERLVSDPAGEVGRVQDFLGLKRVVTDKHFYFNATKG |
| | FPCLKKAQGGSRPRCLGKSKGRPHPRVPQAXVRRLQEFYRPFNRRFYQMTGQDFGWG |

Further analysis of the NOV93a protein yielded the following properties shown in Table 93B.

TABLE 93B
Protein Sequence Properties NOV93a

| | |
|---|---|
| PSort | 0.6000 probability located in plasma membrane; 0.4000 |
| analysis: | probability located in Golgi body; 0.3000 probability |
| | located in endoplasmic reticulum (membrane); 0.1000 |
| | probability located in mitochondrial inner membrane |
| SignalP | Likely cleavage site between residues 7 and 8 |
| analysis: | |

A search of the NOV93a protein against the Geneseq database, a proprietary database that contains sequences published in patents and patent publication, yielded several homologous proteins shown in Table 93C.

TABLE 93C
Geneseq Results for NOV93a

| | | NOV93a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB95507 | Human protein sequence SEQ ID | 31 . . . 253 | 121/229 (52%) | 4e−55 |
| | NO: 18067-Homo sapiens, 390 aa. | 11 . . . 237 | 146/229 (62%) | |
| | [EP1074617-A2, 7 FEB. 2001] | | | |
| AAY17066 | Human 3-OST-3B protein-Homo | 31 . . . 253 | 121/229 (52%) | 4e−55 |
| | sapiens, 390 aa. [WO9922005-A2, | 11 . . . 237 | 146/229 (62%) | |
| | 6 MAY 1999] | | | |
| AAB70115 | Human 3-OST-3B-Homo sapiens, | 31 . . . 253 | 121/230 (52%) | 9e−54 |
| | 391 aa. [WO200113910-A2, | 11 . . . 238 | 146/230 (62%) | |
| | 1 MAR. 2001] | | | |
| AAB70114 | Murine 3-OST-3B-Mus sp, 391 | 31 . . . 253 | 119/231 (51%) | 2e−51 |
| | aa. [WO200113910-A2, | 11 . . . 238 | 147/231 (63%) | |
| | 1 MAR. 2001] | | | |
| AAU12275 | Human PRO5004 polypeptide | 86 . . . 253 | 102/170 (60%) | 9e−48 |
| | sequence-Homo sapiens, 367 aa. | 45 . . . 214 | 117/170 (68%) | |
| | [WO200140466-A2, 7 JUN. 2001] | | | |

In a BLAST search of public sequence databases, the NOV93a protein was found to have homology to the proteins shown in the BLASTP data in Table 93D.

TABLE 93D
Public BLASTP Results for NOV93a

| | | NOV93a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96QI5 | C439A6.1 (NOVEL PROTEIN | 85 . . . 253 | 160/169 (94%) | 2e−89 |
| | SIMILAR TO HEPARAN | 61 . . . 229 | 162/169 (95%) | |
| | SULFATE (GLUCOSAMINE) 3-O- | | | |
| | SULFOTRANSFERASES)-Homo | | | |
| | sapiens (Human), 381 aa (fragment). | | | |
| Q96RX7 | HEPARAN SULPHATE D- | 95 . . . 253 | 153/159 (96%) | 1e−85 |
| | GLUCOSAMINYL 3-O- | 1 . . . 159 | 155/159 (97%) | |
| | SULFOTRANSFERASE-3B LIKE- | | | |
| | Homo sapiens (Human), 311 aa. | | | |
| Q9Y662 | HEPARAN SULFATE D- | 31 . . . 253 | 121/229 (52%) | 1e−54 |
| | GLUCOSAMINYL 3-O- | 11 . . . 237 | 146/229 (62%) | |
| | 2.8.2.23)-Homo sapiens (Human), | | | |
| | 390 aa. | | | |
| Q9QZS6 | D-GLYCOSAMINYL 3-O- | 31 . . . 253 | 119/230 (51%) | 3e−52 |
| | SULFOTRANSFERASE-3B-Mus | 11 . . . 237 | 147/230 (63%) | |
| | musculus (Mouse), 390 aa. | | | |
| Q9Y278 | HEPARAN SULFATE D- | 86 . . . 253 | 102/170 (60%) | 3e−47 |
| | GLUCOSAMINYL 3-O- | 45 . . . 214 | 117/170 (68%) | |
| | SULFOTRANSFERASE-2 (EC | | | |
| | 2.8.2.23)-Homo sapiens (Human), | | | |
| | 367 aa. | | | |

PFam analysis predicts that the NOV93a protein contains the domains shown in the Table 93E.

TABLE 93E
Domain Analysis of NOV93a

| | | | |
|---|---|---|---|
| | | Identities/ | |
| Pfam | NOV93a | Similarities for | Expect |
| Domain | Match Region | the Matched Region | Value |

No Significant Matches Found

The NOV94 clone was analyzed, and the nucleotide and predicted polypeptide sequences are shown in Table 94A.

TABLE 94A
NOV94 Sequence Analysis

| | SEQ ID NO:269 | 2949 bp | |
|---|---|---|---|
| NOV94a, | GTCCGCCTCCCGGCCGCCGAGCCGCAGCCGCCGAGATGCGCGCCGCCCCGGGCCGCGC | | |
| CG59761-01 DNA Sequence | | | |
| | CCCCGCCGGGTCCCGCCCGCCCCGCTGCCGCTGAGCGCATGGGCCCGGACCGCGCCGC | | |
| | GCCGCTCCGGGAGCCGGGCCCGCGGTCCCGCCACCACCCCGCGCGGGACAGATTCATT | | |
| | CACTTTGGAGCTGTAAGTACTGATGTATTAGGGTGCAGCCCTCATTGTTCATTGACGC | | |
| | ACAGTCCCAAAATGAATATCCAAGAGCAGGGTTTCCCCTTCGACCTCGGAGCAAGTTT | | |
| | CACCGAAGATGCTCCCCGACCCCCAGTCCCTCGTGAGGAGCGAGAACTCGTGTCCACA | | |
| | GACCCGAGGCCCGCCAGCTACAGTTTCTGCTCCGGGAAAGGTGTTGGCATTAAAGGTG | | |
| | AGACTTCGACGGCCACTCCGAGGCGCTCGGATCTCGACCTGGGGTATGAGCCTGAGGG | | |
| | CAGTCCCTCCCCCACCCCACCATACTTGAAGTGGGCTGAGTCACTGCATTCCCTGCTG | | |
| | GATGACCAAGATGGGATAAGCCTGTTCAGGACTTTCCTGAAGCAGGAGGGCTGTGCCG | | |
| | ACTTGCTGCACTTCTGGTTTGCCTGCACTGCCTTCAGCAACCTGGAGCCCTGTCACTC | | |
| | GAACGAGGAGAAGAGGCTGAAGCTGGCGAGAGCCATCTACCCAAAGTACATTCTTGAT | | |
| | AACAATGGCATCGTGTCCCGGCAGACCAAGCCAGCCACCAAGAGCTTCATAAAGGGCT | | |
| | GCATCATGAAGCAGCTGATCGATCCTCCCATGTTTGACCAGGCCCAGACCGAAATCCA | | |
| | GGCCACTATGGAGGAAAACACCTATCCCTCCTTCCTTAAGTCTCATATTTATTTGGAA | | |
| | TATACGAGGACAGGCTCGGAGAGCCCCAAAGTCTGTAGTGACCAGACCTCTGCGTCAG | | |
| | GGACAGGGAAGGGCATATCTGGATACCTGCCGACCTTAAATGAAGATGAGGAATGGAA | | |
| | GTGTGACCAGGACATGGATCACGACGATGGCAGAGACGCTGCTCCCCCCGGAAGACTC | | |
| | CCTCAGAAGCTGCTCCTGGAGACAGCTGCCCCGAGGGTCTCCTCCAGTAGACGGTACA | | |
| | GCGAAGGCAGAGAGTTCAGGTATGGATCCTGGCGGGAGCCAGTCAACCCCTATTATGT | | |
| | CAATGCCGGCTATGCCCTGGCCCCAGCCACCAGTGCCAACCACAGCGAGCAGCAGAGC | | |
| | CTGTCCAGCGATGCAGACACCCTGTCCCTCACGGACAGCAGCGTGGATGGGATCCCCC | | |
| | CATACAGGATCCGTAAGCAGCACCGCAGGGAGATGCAGGAGAGCGTGCAGGTCAATGG | | |
| | GCGGGTGCCCCTACCTCACATTCCCCGCACGTACCGGGTGCCGAAGGAGGTCCGCGTG | | |
| | GAGCCTCAGAAGTTCGCGGAGGACCTCATCCACCGCCTGGAGGCTGTGCAGCGCACGC | | |
| | CGGAGGCCGAGGAGAAGCTGGAGGAGCGGCTGAAGCGCGTGCGCATGGAGGAGGAAGG | | |
| | TGAGGACGGCGATCCATCATCAGGGCCCCCAGGGCCGTGTCACAAGCTGCCTCCCGCC | | |
| | CCCGCTTGGCACCACTTCCCGCCCCGCCTGTGTTGGACATGGGCTTGTGCCGGGCTCC | | |
| | GGGATGCACACGAGGAGAACCCTGAGAGCATCCTGGACGAGCACGTACAGCGTGTGCT | | |
| | GACGACACCTGGCCGCCAGTCGCCTGGGCCTGGCCATCGCTCCCCGGACAGTGGGCAC | | |
| | GTGGCCAAGATGCCAGTGGCACTGGGGGGTGCCGCCTCGGGGCACGGGAAGCACGTAC | | |
| | CCAAGTCAGGGGCGAAGCTGGACGCGGCCGGCCTGCACCACCACCGACACGTCCACCA | | |
| | CCACGTCCACCACAGCACAGCCCGGCCCAAGGAGCAGGTGGAGGCCGAGGCCACCCGC | | |
| | AGGGCCCAGAGCACCTTCGCCTCGGGCCTGGAACCACACAGCCATGGCGCAAGGTCCC | | |
| | GAGCCTACTCAGAGAGTGTTCGCGCTGCCCCCAACGCCAGTGATGGCCTCGCCCACAC | | |
| | TGGGAAGGTGGGCGTGGCGTGCAAAAGAAATGCCAAGAAGGCCGAGTCGGGGAAGAGC | | |
| | GCCAGCACCCAGGTGCCAGGTGCCTCGGAGGATGCGGAGAAGAACCAGAAAATCATGC | | |
| | AGTGGATCATTGAGGGGGAAAAGGAGATCAGCAGCCACCGCACCACCGCCCACGGGTC | | |
| | TTCGGGGACCAGGAAGCCACAGCCCCATGAGAACTCCAGACCCTTGTCCCTTGAGCAC | | |
| | CCCTGCCCCGGCCCTCAGCTCCGGACCTCCGTGCAGCCCTCCCACCTCTTCATCCAAG | | |
| | ACCCCACCATGCCACCCCACCCAGCTCCCAACCCCCTAACCCAGCTGGAGGAGGCGCG | | |
| | CCGACGTCTCGAGGAGCAAGAAAACAGAGCCAGCCGAGCACCCTCCAAGCAGACGTAT | | |
| | CTCCAGCAGGTTATGCCGCGCGGACGCGCCTCCGTCAGGCCAGCGTCCGCGCCGGTCC | | |
| | TGCACGTGGTACCAGCCGTGTCGGACATGGAGCTCTCCGAGACAGAGACAAGATCGCA | | |
| | GAGGAAGGTCCGCGGCGGGAGTGCCCAGCCGTGTGACAGCATCGTTGTGGCCTACTAC | | |
| | TTCTGCGGGGAACCCATCCCCTACCGCACCCTGGTGAGGGGCCGCGCTGTCACCCTGG | | |
| | GCCAGTTCAAGGAGCTGCTGACCAAAAAGGGCAGCTACAGATACTACTTCAAGAAAGT | | |
| | GAGCGACGAGTTTGACTGTGCGGTGGTGTTTGAGGAGGTTCGAGAGGACGAGGCCGTC | | |
| | CTGCCCGTCTTTGAGGAGAACATCATCGGCAAAGTGGAGAAGGTGGACTGATAGGCTG | | |
| | GTGGGCTGGCCCCTGTGCCAGGCGACCCCCTTGGCGGGCACGGGTGTCACGGCCAGGC | | |
| | AGATCACCTCGTACTCAGGAGCCCGATGGGGAACAGTGTTGCCTGTACC | | |
| | ORF Start: ATG at 97 | | ORF Stop: TGA at 2833 |
| | SEQ ID NO:270 | 912 aa | MW at 101118.1 kD |

| | |
|---|---|
| NOV94a, | MGPDRAAPLREPGPGSRHHRARDRLIHFGAVSTDVLGCSAHCSLTQSPKMNIQEQGFP |
| CG59761-01 Protein Sequence | |
| | LDLGASFTEDAPRPPVPGEEGELVSTDPRPASYSFCSGKGVGIKGETSTATPRRSDLD |
| | LGYEPEGSASPTPPYLKWAESLHSLLDDQDGISLFRTFLKQEGCADLLDFWFACTGFR |
| | KLEPCDSNEEKRLKLARAIYRKYILDNNGIVSRQTKPATKSFIKGCIMKQLIDPAMFD |
| | QAQTEIQATMEENTYPSFLKSDIYLEYTRTGSESPKVCSDQSSGSGTGKGISGYLPTL |
| | NEDEEWKCDQDMDEDDGRDAAPPGRLPQKLLLETAAPRVSSSRRYSEGREFRYGSWRE |
| | PVNPYYVNAGYALAPATSANDSEQQSLSSDADTLSLTDSSVDGIPPYRIRKQHRREMQ |
| | ESVQVNGRVPLPHIPRTYRVPKEVRVEPQKFAEELIHRLEAVQRTREAEEKLEERLKR |
| | VRMEEEGEDGDPSSGPPGPCHKLPPAPAWHHFPPRLCWTWACAGLRDAHEENPESILD |
| | EHVQRVLRTPGRQSPGPGHRSPDSGHVAKMPVALGGAASGHGKHVPKSGAKLDAAGLH |
| | HHRHVHHHVHHSTARPKEQVEAEATRRAQSSFAWGLEPHSHGARSRGYSESVGAAPNA |
| | SDGLAHSGKVGVACKRNAKKAESGKSASTEVPGASEDAEKNQKIMQWIIEGEKEISRH |
| | RRTGHGSSGTRKPQPHENSRPLSLEHPWAGPQLRTSVQPSHLFIQDPTMPPHPAPNPL |
| | TQLEEARRRLEEEEKRASRAPSKQRYVQEVMRRGRACVRPACAPVLHVVPAVSDMELS |
| | ETETRSQRKVGGGSAQPCDSIVVAYYFCGEPIPYRTLVRGRAVTLGQFKELLTKKGSY |
| | RYYFKKVSDEFDCGVVFEEFREDEAVLPVFEEKIIGKVEKVD |

Further analysis of the NOV94a protein yielded the following properties shown in Table 94B.

TABLE 94B
Protein Sequence Properties NOV94a

| | |
|---|---|
| PSort | 0.6000 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV94a protein against the Geneseq database, a proprietary database that contains sequences published in patents and patent publication, yielded several homologous proteins shown in Table 94C.

TABLE 94C
Geneseq Results for NOV94a

| | | NOV94a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG68175 | Wnt signaling protein SEQ ID | 13 . . . 912 | 898/900 (99%) | 0.0 |
| | NO: 91-Homo sapiens, 900 aa. | 1 . . . 900 | 898/900 (99%) | |
| | [WO200177327-A1, | | | |
| | 18 OCT. 2001] | | | |
| AAW96264 | Human axin-Homo sapiens, 900 | 13 . . . 912 | 898/900 (99%) | 0.0 |
| | aa. [WO9902179-A1, | 1 . . . 900 | 898/900 (99%) | |
| | 21 JAN. 1999] | | | |
| AAW96265 | Murine axin-Mus musculus, 992 | 6 . . . 912 | 781/914 (85%) | 0.0 |
| | aa. [WO9902179-A1, | 84 . . . 992 | 820/914 (89%) | |
| | 21 JAN. 1999] | | | |
| AAW93569 | Human conductin protein-Homo | 60 . . . 912 | 378/892 (42%) | e−171 |
| | sapiens, 840 aa. [WO9911780-A2, | 12 . . . 840 | 506/892 (56%) | |
| | 11 MAR. 1999] | | | |
| AAW93570 | Human conductin protein-Homo | 60 . . . 912 | 378/892 (42%) | e−171 |
| | sapiens, 840 aa. [WO9911780-A2, | 12 . . . 840 | 506/892 (56%) | |
| | 11 MAR. 1999] | | | |

In a BLAST search of public sequence databases, the NOV94a protein was found to have homology to the proteins shown in the BLASTP data in Table 94D.

TABLE 94D
Public BLASTP Results for NOV94a

| | | NOV94a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O15169 | Axin 1 (Axis inhibition protein 1) | 13 . . . 912 | 898/900 (99%) | 0.0 |
| | (hAxin)-Homo sapiens (Human), | 1 . . . 900 | 898/900 (99%) | |
| | 900 aa (fragment). | | | |
| Q96S28 | AXIN-Homo sapiens (Human), | 50 . . . 912 | 858/863 (99%) | 0.0 |
| | 862 aa. | 1 . . . 862 | 858/863 (99%) | |
| O35625 | Axin 1 (Axis inhibition protein 1) | 6 . . . 912 | 781/914 (85%) | 0.0 |
| | (Fused protein)-Mus musculus | 84 . . . 992 | 820/914 (89%) | |
| | (Mouse), 992 aa (fragment). | | | |
| O70239 | Axin 1 protein (Axis inhibition | 6 . . . 912 | 756/914 (82%) | 0.0 |
| | protein 1) (rAxin)-Rattus | 21 . . . 893 | 793/914 (86%) | |
| | norvegicus (Rat), 893 aa | | | |
| | (fragment). | | | |
| T08422 | negative regualtor axin [imported]- | 46 . . . 912 | 726/872 (83%) | 0.0 |
| | rat, 832 aa. | 2 . . . 832 | 760/872 (86%) | |

PFam analysis predicts that the NOV94a protein contains the domains shown in the Table 94E.

TABLE 94E
Domain Analysis of NOV94a

| | | Identities/ | |
|---|---|---|---|
| Pfam | NOV94a | Similarities for | Expect |
| Domain | Match Region | the Matched Region | Value |
| RGS: domain 1 of 2 | 137 . . . 198 | 23/75 (31%) | 5.6e−06 |
| | | 44/75 (59%) | |
| RGS: domain 2 of 2 | 231 . . . 260 | 13/30 (43%) | 0.12 |
| | | 21/30 (70%) | |
| TP2: domain 1 of 1 | 585 . . . 709 | 33/147 (22%) | 9.6 |
| | | 52/147 (35%) | |
| DIX: domain 1 of 1 | 830 . . . 912 | 40/86 (47%) | 5.6e−44 |
| | | 83/86 (97%) | |

### Example 95

The NOV95 clone was analyzed, and the nucleotide and predicted polypeptide sequences are shown in Table 95A.

TABLE 95A
NOV95 Sequence Analysis

| | SEQ ID NO:271 | 2223 bp | |
|---|---|---|---|
| NOV95a, | TTGCAGGCATCACCCACGCCCTCTGCACCCACGCTGGAGGACGGGGAGGTTGTCAGGG | | |
| CG59756-01 DNA Sequence | | | |
| | GCTATGATGAGATGAGTCGGGGCCGCTTCGACTTTGATGATGGAGGGGCGTACTGCGG | | |
| | GGGCTGGGAGGGGGGAAAGGCCCATGGGCATGGACTGTGCACAGGCCCCAAGGGCCAG | | |
| | GGCGAATACTCTCGCTCCTGGAACTTTGGCTTTGAGGTGGCAGGTGTCTACACCTCGC | | |
| | CCAGCGGAAACACCTTTGAGGGATACTGGAGCCAGGGCAAACGGCATGGGCTGGGCAT | | |
| | AGAGACCAAGGGGCGCTGGCTCTACAAGGCCGAGTGGACACATGGCTTCAAGGGACGC | | |
| | GCCTGCAAGACGGCTATGGCACCGAGACCTATGCTGATGGAGGGACGTACCAAGGCCA | | |
| | GTTCACCAACGGCATGCGCCATGGCTACGGAGTACGCCAGAGCGTGCCCTACGGGATG | | |
| | GCAACCGCACGGTGGCCCCGGACTCTCCCGCCTCGCCGGCCTCCGACGGCCCCGCGCT | | |
| | GCCCTCGCCCGCCATCCCGCGTGGCGGCTTCGCGCTCAGCCTCCTGGCCAATGCCGAG | | |
| | GCGGCCGCGCGGGCGCCCAAGGGCGGCGGCCTCTTCCAGCGGGCCGCGCTGCTGGGCA | | |
| | ACCTGCGGCGCGCAGAGTCGCGCACGTCCGTGGGTAGCCAGCGCAGCCGTGTCAGCTT | | |
| | CCTTAAGAGCGACCTCAGCTCGGGCGCCAGCGACGCCGCGTCCACCGCCAGCCTGCGA | | |
| | GAGGCCGCCGAGGGCGCCGACGAGGCCGCACCCTTCGAGGCCGATATCGACGCCACCA | | |
| | CCACCGAGACCTACATGGGCGAGTGGAAGAACGACAAACGCTCGGGCTTCGGCGTGAC | | |
| | CGAACGCTCCAGTGGCCTCCGCTACGAGGGCGAGTGGCTGGACAACCTGCGCCACGGC | | |
| | TATGGCTCCACCACGCTGCCCGACGGCCACCCCGAGGAGGGCAAGTACCGCCACAACG | | |
| | TGCTGGTCAAGGACACCAAGCGCCGCATGCTGCAGCTCAAGAGCAACAAGGTCCGCCA | | |
| | GAAAGTGGAGCACAGTGTGGAGGGTGCCCAGCGCGCCGCTGCTATCGCGCCCCAGAAG | | |
| | GCCGAGATTGCCGCCTCCAGGACAAGCCACGCCAAGGCCAAAGCTGAGGCAGCGGAAC | | |
| | AGGCCGCCCTGGCTGCCAACCAGGACTCCAACATTGCTCGCACTTTGGCCAGGGAGCT | | |
| | GGCTCCGGACTTCTACCAGCCAGGTCCGGAATATCAGAAGCGCCGGCTGCTGCAGGAG | | |
| | ATCCTGGAGAACTCGGAGAGCCTGCTGGAGCCCCCCGACCGGGGCGCCGGCGCAGCGG | | |
| | GCCTCCGACAGCCGCCCCGCGAGAGCCCGCAGCTGCACGAGCGTGAGACCCCTCGGCC | | |
| | CGAGGGTCCCTCCCCGTCACCGGCCGGGACGCCCCCGCAGCCCAAGCGGCCCAGGCCC | | |
| | GGGGTGTCCAAGGACCGCCTGCTGAGCCCAGGCGCCTGGAACGGCGAGCCCAGCGGTG | | |
| | AGGGCAGCCGGTCAGTCACTCCGTCCGAGGGCGCGGGCCGCCGCAGCCCCGCGCGTCC | | |
| | AGCCACCGAGCGCATGGCCATCGAGGCTCTGCAGGCACCGCCTGCGCCGTCGCGGGAG | | |
| | CCGGAGGTGGCGCTTTACCAGGGCTACCACACCTATGCTGTGCGCACCACCCCGCCCG | | |
| | AGCCCCCACCCTTTGACGACCAGCCCGACCCCGACGTCTCCGGGTCCGAGTCCGCGCC | | |
| | CTCGTCCCCCGCCACCGCCCCCCTGCAGGCCCCCACCCTCCGAGGCCCCGAGCCTGCA | | |
| | CGCGAGACCCCCGCCAAGCTGGAGCCCAAGCCCATCATCCCCAAAGCCGAGCCCAGGG | | |
| | CCAAGGCCCGCAAGACTGAGGCTCCAGGGCTGACCAAGGCGGGGGCCAAGAAGAAGGC | | |
| | GCGGAAGGAGGCCGCACTGGCGGCAGAGGCGGAGGTGGAGGTGGAAGAGGTCCCCAAC | | |
| | ACCATCCTCATCTGCATGGTGATCCTGCTGAACATCGGCCTGGCCATCCTCTTTGTTC | | |
| | ACCTCCTGACCTGACCGTCGCTTACCAGGTGCAGCCAGCTGGCTGCAGGAGGGGTTGG | | |
| | GGGGCAGGAGCCCCTGGGG | | |
| | ORF Start: ATG at 70 | | ORF Stop: TGA at 2158 |
| | SEQ ID NO:272 | 696 aa | MW at 74220.7 kD |

| | |
|---|---|
| NOV95a, | MSGGRFDFDDQCAYCGGWEGGKAEGHQLCTGPKCQGEYSGSFGFEVAGVYTWPSGN |
| CG59756-01 Protein Sequence | |
| | TFEGYWSQGKRHGLGIETKGRWLYKGEWTHGFKGRYGIRQSSSSGAKYEGTWNNGLQD |
| | GYGTETYADGGTYQGQFTNGMRHGYCVRQSVPYGMAVVVRSPLRTSLSSLRSEHSNGT |
| | VAPDSPASPASDCPALPSPAIPRGGFALSLLANAEAAAPAPKGGGLFQRGAILGRLRR |
| | AESRTSVGSQRSRVSFLKSDLSSGASDAASTASLGEAAEGADEAAPFEADTDATTTET |
| | YMGEWKNDKRSGFGVSERSSGLRYEGEWLDNLRHGYGCTTLPDGHREEGKYRHNVLVK |
| | DTKRRMLQLKSNKVRQKVEHSVEGAQRAAAIARQKAEIAASRTSHAKAKAEAAEQAAL |
| | AANQESNIARTLARELAPDFYQPGPEYQKRRLLQEILENSESLLEPPDRGAGAAGLPQ |
| | PPRESPQLHERETPRPEGGSPSPAGTPPQPKRPRPGVSKDGLLSPGAWNGEPSGEGSR |
| | SVTPSEGAGRRSPARPATERMAIEAIQAPPAPSREPEVALYQGYHSYAVRTTPPEPPP |
| | FEDQPEPEVSGSESAPSSPATAPLQAFTLRGPEPARETPAKLEPKPIIPKAEPRAKAR |
| | KTEARCLTKACAKKKARKEAALAAEAEVEVEEVPNTILICMVILLNICLAILFVHLLT |

Further analysis of the NOV95a protein yielded the following properties shown in Table 95B.

TABLE 95B
Protein Sequence Properties NOV95a

| | |
|---|---|
| PSort | 0.8000 probability located in nucleus; 0.7000 probability |
| analysis: | located in plasma membrane; 0.3133 probability located in |
| | microbody (peroxisome); 0.2000 probability located in |
| | endoplasmic reticulum (membrane) |
| SignalP | No Known Signal Sequence Predicted |
| analysis: | |

A search of the NOV95a protein against the Geneseq database, a proprietary database that contains sequences published in patents and patent publication, yielded several homologous proteins shown in Table 95C.

TABLE 95C
Geneseq Results for NOV95a

| | | NOV95a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM79123 | Human protein SEQ ID NO 1785- | 3 . . . 696 | 293/704 (41%) | e−127 |
| | Homo sapiens, 628 aa. | 4 . . . 628 | 377/704 (52%) | |
| | [WO200157190-A2, 9 AUG. 2001] | | | |
| AAM80107 | Human protein SEQ ID NO 3753- | 283 . . . 696 | 146/421 (34%) | 2e−43 |
| | Homo sapiens, 378 aa. | 24 . . . 378 | 194/421 (45%) | |
| | [WO200157190-A2, 9 AUG. 2001] | | | |
| ABB21683 | Protein #3682 encoded by probe for | 257 . . . 389 | 78/133 (58%) | 7e−42 |
| | measuring heart cell gene expression- | 6 . . . 135 | 104/133 (77%) | |
| | Homo sapiens, 135 aa. | | | |
| | [WO200157274-A2, 9 AUG. 2001] | | | |
| AAM57089 | Human brain expressed single exon | 257 . . . 389 | 78/133 (58%) | 7e−42 |
| | probe encoded protein SEQ ID NO: | 6 . . . 135 | 104/133 (77%) | |
| | 29194-Homo sapiens, 135 aa. | | | |
| | [WO200157275-A2, 9 AUG. 2001] | | | |
| AAM17323 | Peptide #3757 encoded by probe for | 257 . . . 389 | 78/133 (58%) | 7e−42 |
| | measuring cervical gene expression- | 6 . . . 135 | 104/133 (77%) | |
| | Homo sapiens, 135 aa. | | | |
| | [WO200157278-A2, 9 AUG. 2001] | | | |

In a BLAST search of public sequence databases, the NOV95a protein was found to have homology to the proteins shown in the BLASTP data in Table 95D.

TABLE 95D
Public BLASTP Results for NOV95a

| | | NOV95a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9GKY7 | JUNCTOPHILIN TYPE 2- | 1 . . . 696 | 644/701 (91%) | 0.0 |
| | Oryctolagus cuniculus (Rabbit), 694 aa. | 1 . . . 694 | 662/701 (93%) | |
| Q9ET79 | JUNCTOPHILIN TYPE 2-Mus | 1 . . . 696 | 608/706 (86%) | 0.0 |
| | musculus (Mouse), 696 aa. | 1 . . . 696 | 644/706 (91%) | |
| Q9BR39 | DJ1108D11.1 (NOVEL PROTEIN | 128 . . . 672 | 544/545 (99%) | 0.0 |
| | SIMILAR TO C. ELEGANS T22C1.7)- | 1 . . . 545 | 544/545 (99%) | |
| | Homo sapiens (Human), 552 aa | | | |
| | (fragment). | | | |
| Q9GKY8 | MITSUGUMIN72/JUNCTOPHILIN | 1 . . . 696 | 364/704 (51%) | 0.0 |
| | TYPE1-Oryctolagus cuniculus | 1 . . . 662 | 468/704 (65%) | |
| | (Rabbit), 662 aa. | | | |
| Q9ET80 | JUNCTOPHILTN TYPE 1-Mus | 1 . . . 696 | 371/707 (52%) | 0.0 |
| | musculus (Mouse), 660 aa. | 1 . . . 660 | 469/707 (65%) | |

PFam analysis predicts that the NOV95a protein contains the domains shown in the Table 95E.

TABLE 95E
Domain Analysis of NOV95a

| | | Identities/ | |
|---|---|---|---|
| Pfam | NOV95a | Similarities for | Expect |
| Domain | Match Region | the Matched Region | Value |
| MORN: domain 1 of 7 | 14 . . . 36 | 10/23 (43%) | 1.1 |
| | | 13/23 (57%) | |
| MORN: domain 2 of 7 | 38 . . . 59 | 9/23 (39%) | 0.31 |
| | | 15/23 (65%) | |
| MORN: domain 3 of 7 | 60 . . . 77 | 8/23 (35%) | 3 |
| | | 15/23 (65%) | |
| MORN: domain 4 of 7 | 106 . . . 128 | 11/23 (48%) | 3.7e−06 |
| | | 20/23 (87%) | |
| MORN: domain 5 of 7 | 129 . . . 151 | 8/23 (35%) | 0.027 |
| | | 15/23 (65%) | |
| MORN: domain 6 of 7 | 291 . . . 313 | 12/23 (52%) | 0.00056 |
| | | 19/23 (83%) | |
| MORN: domain 7 of 7 | 314 . . . 336 | 11/23 (48%) | 0.00022 |
| | | 19/23 (83%) | |

### Example 96

The NOV96 clone was analyzed, and the nucleotide and predicted polypeptide sequences are shown in Table 96A.

TABLE 96A
NOV96 Sequence Analysis

| | SEQ ID NO:273 | 3257 bp | |
|---|---|---|---|
| NOV96a, | CGTAGGCGCTTCGGCCATGACTGCGGAGCTGCAGCAGGACGACGCGGCCGGCGCGGCA | | |
| CG59708-01 DNA Sequence | | | |
| | GACGGCCACGGCTCGAGCTGCCAAATGCTGTTAAATCAACTGAGAGAAATCACAGGCA | | |
| | TTCAGGACCCTTCCTTTCTCCATGAAGCTCTGAAGGCCAGTAATCGTCACATTACTCA | | |
| | GGCAGTCAGCCTTCTCACTGATGAGAGAGTTAAGGAGCCCAGTCAAGACACTGTTGCT | | |
| | ACAGAACCATCTGAACTAGAGGCGACTGCTGCCAACAAGGAAGTATTAGCAAAAGTTA | | |
| | TAGACCTTACTCATGATAACAAAGATGATCTTCAGGCTGCCATTGCTTTGAGTCTACT | | |
| | GGAGTCTCCCAAAATTCAAGCTGATGGAAGAGATCTTAACAGGATGCATGAAGCAACC | | |
| | TCTGCAGAAACTAAACGCTCAAAGAGAAAACGCTGTGAAGTCTGGGGAGAAAACCCCA | | |
| | ATCCCAATGACTGGAGGAGAGTTGATGGTTGGCCAGTTGGGCTGTAAAATGTTGGCAA | | |
| | TACATGTTGGTTTAGTGCTGTTATTCAGTCTCTCTTTCAATTGCCTCAATTTCGAAGA | | |
| | CTTGTTCTCAGTTATAGTCTGCCACAAAATGTACTTGAAAATTGTCGAAGTCATACAG | | |
| | AAAAGAGAAATATCATGTTTATGCAAGAGCTTCAGTATTTGTTTGCTCTAATGATGGG | | |
| | ATCAAATAGAAAATTTGTAGACCCCTCTGCAGCCCTGGATCTATTAAAGGGAGCATTC | | |
| | CGATCATCTGAGGAACAGCAGCAAGATGTGAGTGAATTCACACACAACCTCCTGGATT | | |
| | GGCTAGACCACGCATTCCAGCTACCTGTTAATGTTAACAGTCCCAGGAACAAATCTGA | | |
| | AAATCCAATGGTGCAGCTGTTCTATGGTACTTTCCTGACTGAAGGGCTTCGTGAAGGA | | |
| | AAACCCTTTTGTAACAATGAGACCTTCGGCCAGTATCCTCTTCAGCTAAACGGTTATC | | |
| | GCAACTTACACGAGTGTTTGCAAGGGGCCATGGTGCAGGGTGATGTTCAGCTTCTTCC | | |
| | CTCCGATCACTCGGTGAAGTATGGACAAGAGCGTTGGTTTACAAAGCTACCTCCAGTG | | |
| | TTCACCTTTGAACTCTCAAGATTTGAGTTTAATCACTCCCTTGGGCAGCCAGAGAAAA | | |
| | TTCACAATAAGCTGGAATTTCCTCAGATTATTTATATGGACACCTACATGTACACGAG | | |
| | CAAGGAGCTTATTCGAAATAAGAGAGAGTGTATTCGAAAGTTGAAGGAGGAAATAAAA | | |
| | ATTCTGCAGCAAAAATTGGAAAGGTATGTGAAATATCGCTCAGGCCCAGCTCGCTTCC | | |
| | CGCTCCCGGACATGCTGAAATATGTTATTGAATTTGCTAGTACAAAACCTGCCTCAGA | | |
| | AAGCTGTCCACCTGAAAGTGACACACATATGACATTACCACTTTCTTCAGTGCACTGC | | |
| | TCGGTTTCTGACCAGACATCCAAGGAAAGTACAAGTACAGAAAGCTCTTCTCAGGATG | | |
| | TTGAAAGTACCTTTTCTTCTCCTGAAGATTCTTTACCCAAGTCTAAACCACTGACATC | | |
| | TTCTCGGTCTTCCATGGAAATGCCTTCACAGCCAGCTCCACGAACAGTCACAGATGAG | | |
| | GAGATAAATTTTGTTAAGACCTCTCTTCAGAGATGGAGGACTGAGATTGAACAAGATA | | |
| | TACAAGATTTAAAGACTTGTATTGCAAGTACTACTCAGACTATTGAACAGATGTACTG | | |
| | CGATCCTCTCCTTCGTCAGGTGCCTTATCGCTTGCATGCAGTTCTTGTTCATGAAGGA | | |
| | CAAGCAAATCCTGGACACTATTGGGCCTATATCTATAATCAACCCCGACAGAGCTGGC | | |
| | TCAAGTACAATGACATCTCTGTTACTGAATCTTCCTGGGAAGAAGTTGAAAGAGATTC | | |
| | CTATGGAGGCCTGAGAAATGTTAGTCCTTACTGTCTGATGTACATTAATGCCAAACTA | | |
| | CCCTACTTCAATCCAGAGGCAGCCCCAACTGAATCAGATCAAATGTCAGAAGTGGAAG | | |
| | CCCTATCTGTGGAACTCAAGCATTACATTCAGGAGGATAACTGGCGGTTTGAGCAGGA | | |
| | AGTAGAGGAGTGGGAAGAAGAGCAGTCTTGCAAAATCCCTCAAATGGAGTCCTCCCCC | | |
| | AACTCCTCATCACAGGGCTACTCTACATCACAAGAGCCTTCAGTAGCCTCTTCTCATG | | |
| | GGGTTCGCTGCTTGTCATCTGAGCATGCTGTGATTGTAAAGGAGCAAACTGCCCAGGC | | |
| | TATTGCAAACACAGCCCGTGCCTATGAGAAGAGCGGTGTAGAAGCGGCACTGAGTGAG | | |
| | GCATTCCATGAAGAATACTCCAGGCTCTATCAGCTTGCCAAAGAGACCCCCACCTCTC | | |
| | ACAGTGATCCTCGACTTCAGCATGTCCTTGTCTACTTTTTCCAAAATGAAGCACCCAA | | |
| | AAGGGTAGTAGAACGAACGCTTCTGGAACAGTTTGCAGATAAAAATCTTAGCTATGAT | | |
| | GAAAGATCAATCAGCATTATGAAGGTGGCTCAAGCGAAACTGAAGGAAATTGGTCCAG | | |
| | ATGAGATGAATATGGAAGAGTACAAGAGGTGGCATGAAGATTATAGTTTGTTCCGAAA | | |
| | AGTGTCTGTGTATCTCCTAACAGCCCTACAACTCTATCAAAAAGGAAAGTACCAAGAC | | |
| | GCACTTTCCTACCTGGTATATGCCTACCAGAGCAATGCTGCCCTGCTGATGAAGGGGC | | |
| | GGCATTAATGTGATGAATGAACTGATCATCCCCTGCATTCACCTTATCATTAATAATG | | |
| | ACATTTCCAAGGATGATCTGGATGCCATTGAGGTCATGAGAAACCATTGGTGCTCTTA | | |
| | CCTTGGGCAAGATATTGCAGAAAATCTGCAGCTGTGCCTAGGCGAGTTTCTACCCAGA | | |
| | CTTCTAGATCCTTCTGCAGAAATCATCGTCTTGAAAGAGCCTCCAACTATTCGACCCA | | |
| | ATTCTCCCTATGACCTATGTAGCCGATTTGCAGCTGTCATGGAGTCAATTCAGGGAGT | | |
| | TTCAACTGTGACAGTGAAATAAGCTCCCACATGTTCAAGGCCCATTCTGGTTCCTGGC | | |
| | TGCCTGCCTCTTGCACACAAGTTCGTTGTCATAGTCCTCACCTTGGGAAAAGGATTAG | | |
| | GTGGGCACA | | |
| | ORF Start: ATG at 17 | | ORF Stop: TAA at 3152 |
| | SEQ ID NO:274 | 1045 aa | MW at 119041.7 kD |
| NOV96a, | MTAELQQDDAAGAADGHGSSCQMLLNQLREITGIQDPSFLHEALKASNGDITQAVSLL | | |
| CG59708-01 Protein | | | |
| Sequence | TDERVKBPSQDTVATEPSEVEGSAANKEVLAKVTDLTHDNKDDLQAAIALSLLESPKI | | |
| | QADGRDLNRMHEATSAETKRSKRKRCEVWGENPNPNDWRRVDGWPVGLKNVGNTCWFS | | |
| | AVIQSLFQLPEFRRLVLSYSLPQNVLENCRSHTEKRNIMFMQELQYLFALMMGSNRKF | | |
| | VDPSAALDLLKGAFRSSEEQQQDVSEFTHKLLDWLEDAFQLAVNVNSPRNKSENPMVQ | | |
| | LFYGTFLTEGVREGKPFCNNETFGQYPLQVNGYRNLDECLEGAMVEGDVELLPSDHSV | | |
| | KYGQERWFTKLPPVLTFELSRFEFNQSLGQPEKIHNKLEFPQIIYMDRYMYRSKELIR | | |
| | NKRECIRKLKEEIKILQQKLERYVKYGSGPARFPLPDMLKYVIEFASTKPASESCPPE | | |
| | SDTHMTLPLSSVHCSVSDQTSKESTSTESSSQDVESTFSSPEDSLPKSKPLTSSRSSM | | |
| | EMPSQPAPRTVTDEEINFVKTCLQRWRSEIEQDIQDLKTCIASTTQTIEQMYCDPLLR | | |
| | QVPYRLHAVLVHEGQANAGHYWAYIYNQPRQSWLKYNDISVTESSWEEVERDSYGGLR | | |
| | NVSAYCLMYINAKLPYFNAEAAPTESDQMSEVEALSVELKHYIQEDNWRFEQEVEEWE | | |
| | EEQSCKIPQMESSPNSSSQGYSTSQEPSVASSHGVRCLSSEHAVIVKEQTAQAIANTA | | |
| | RAYEKSGVEAALSEAFHEEYSRLYQLAKETPTSHSDPRLQHVLVYFFQNEAPKRVVER | | |
| | TLLEQFADKNLSYDERSISIMKVAQAKLKEIGPDDMNMEEYKRWHEDYSLFRKVSVYL | | |
| | LTGLELYQKGKYQEALSYLVYAYQSNAALLMKGPRRGVKESVIALYRRKCLLELNAKA | | |
| | ASLFETNDDHSVTEGINVMNELIIPCIHLIINNDISKDDLDAIEVMRNHWCSYLGQDI | | |
| | AENLQLCLGEFLPRLLDPSAEIIVLKEPPTIRPNSPYDLCSRFAAVMESIQGVSTVTVK | | |
| | SEQ ID NO:275 | 3044 bp | |
| NOV96b, | CGTAGGCGCTTCGGCCATGACTGCGGAGCTGCAGCAGGACGACGCGGCCGGCGCGGCA | | |
| CG59708-02 DNA Sequence | | | |
| | GACGGCCACGGCTCGAGCTGCCAAATGCTGTTAAATCAACTGAGAGAAATCACAGGCA | | |
| | TTCAGGACCCTTCCTTTCTCCATGAAGCTCTGAAGGCCAGTAATGGTGACATTACTCA | | |
| | GGCAGTCAGCCTTCTCACTGATGAGAGAGTTAAGGAGCCCAGTCAAGACACTGTTGCT | | |
| | ACAGAACCATCTGAAGTAGAGGGGAGTGCTGCCAACAAGGAAGTATTAGCAAAAGTTA | | |
| | TAGACCTTACTCATGATAACAAAGATGATCTTCAGGCTGCCATTGCTTTGAGTCTACT | | |
| | GGAGTCTCCCAAAATTCAAGCTGATGGAAGAGATCTTAACAGGATGCATGAAGCAACC | | |
| | TCTGCAGAAACTAAACGCTCAAAGAGAAATATCATGTTTATGCAAGAGCTTCAGTATT | | |
| | TGTTTGCTCTAATGATGGGATCAAATAGAAAATTTGTAGACCCGTCTGCAGCCCTGGA | | |
| | TCTATTAAAGGGAGCATTCCGATCATCTGAGGAACAGCAGCAAGATGTGAGTGAATTC | | |
| | ACACACAAGCTCCTGGATTGGCTAGAGGACGCATTCCAGCTAGCTGTTAATGTTAACA | | |
| | GTCCCAGGAACAAATCTGAAAATCCAATGGTGCAGCTGTTCTATGGTACTTTCCTGAC | | |
| | TGAAGGGGTTCGTGAAGGAAAACCCTTTTGTAACAATGAGACCTTCGGCCAGTATCCT | | |
| | CTTCAGGTAAACGGTTATCGCAACTTAGACGAGTGTTTGGAAGGGGCCATGGTGGAGG | | |
| | GTGATGTTGAGCTTCTTCCCTCCGATCACTCGGTGAAGTATGGACAAGAGCGTTGGTT | | |
| | TACAAAGCTACCTCCAGTGTTGACCTTTGAACTCTCAAGATTTGAGTTTAATCAGTCC | | |
| | CTTGGGCAGCCAGAGAAAATTCACAATAAGCTGGAATTTCCTCAGATTATTTATATGG | | |
| | ACAGGTACATGTACAGGAGCAAGGAGCTTATTCGAAATAAGAGAGAGTGTATTCGAAA | | |
| | GTTGAAGGAGGAAATAAAAATTCTGCAGCAAAAATTGGAAAGGTATGTGAAATATGGC | | |
| | TCAGGCCCAGCTCCGTTCCCGCTCCCGGACATGCTGAAATATGTTATTGAATTTGCTA | | |
| | GTACAAAACCTGCCTCAGAAAGCTGTCCACCTGAAAGTGACACACATATGACATTACC | | |
| | ACTTTCTTCAGTGCACTGCTCGGTTTCTGACCAGACATCCAAGGAAACTACAAGTACA | | |
| | GAAACCTCTTCTCAGGATGTTGAAAGTACCTTTTCTTCTCCTGAAGATTCTTTACCCA | | |
| | AGTCTAAACCACTGACATCTTCTCGGTCTTCCATGGAAATGCCTTCACAGCCAGCTCC | | |
| | ACGAACAGTCACAGATGAGCAGATAAATTTTGTTAAGACCTGTCTTCAGACATGGAGG | | |
| | AGTGAGATTGAACAAGATATACAAGATTTAAAGACTTGTATTGCAAGTACTACTCAGA | | |
| | CTATTGAACAGATGTACTGCGATCCTCTCCTTCGTCAGGTGCCTTATCGCTTGCATGC | | |
| | AGTTCTTGTTCATGAACGACAAGCAAATGCTGGACACTATTCGGCCTATATCTATAAT | | |
| | CAACCCCGACAGAGCTGGCTCAAGTACAATGACATCTCTGTTACTGAATCTTCCTGGG | | |
| | AACAAGTTCAAAGAGATTCCTATGGAGGCCTGAGAAATGTTACTGCTTACTGTCTCAT | | |
| | CTACATTAATCCCAAACTACCCTACTTCAATGCAGAGGCAGCCCCAACTGAATCAGAT | | |
| | CAAATGTCAGAACTGGAAGCCCTATCTCTGGAACTCAAGCATTACATTCACGAGGATA | | |
| | ACTGGCGGTTTGAGCAGGAAGTAGAGGAGTGGGAAGAAGAGCAGTCTTGCAAAATCCC | | |
| | TCAAATCGAGTCCTCCCCCAACTCCTCATCACAGGCCTACTCTACATCACAAGAGCCT | | |
| | TCAGTAGCCTCTTCTCATGGGGTTCGCTGCTTGTCATCTGAGCATGCTGTGATTGTAA | | |
| | AGGAGCAAACTGCCCAGGCTATTGCAAACACAGCCCGTGCCTATGAGAAGAGCGGTGT | | |
| | AGAAGCGGCACTGAGTGAGGCATTCCATGAAGAATACTCCAGGCTCTATCAGCTTGCC | | |
| | AAAGAGACCCCCACCTCTCACAGTGATCCTCGACTTCAGCATGTCCTTGTCTACTTTT | | |
| | TAAAAATCTTAGCTATGATCAAAGATCAATCAGCATTATGAAGGTGGCTCAAGCGAAA | | |
| | CTGAAGGAAATTGGTCCAGATGACATGAATATGGAAGAGTACAAGAGGTGGCATGAAG | | |
| | ATTATAGTTTGTTCCGAAAAGTGTCTGTGTATCTCCTAACAGGCCTAGAACTCTATCA | | |
| | AAAAGGAAAGTACCAAGAGGCACTTTCCTACCTGGTATATCCCTACCAGAGCAATGCT | | |
| | GCCCTGCTGATGAACCGGCCCCGCCGGCGGCTCAAAGAATCCGTCATTGCTTTATACC | | |
| | GAAGAAAATGCCTTCTCGAGCTGAATGCCAAAGCAGCTTCTCTTTTTGAAACAAATGA | | |
| | CACCTTATCATTAATAATGACATTTCCAAGGATGATCTGGATGCCATTGAGGTCATGA | | |
| | GAAACCATTGGTCCTCTTACCTTGGGCAAGATATTGCAGAAAATCTGCAGCTGTGCCT | | |
| | AGGGGAGTTTCTACCCAGACTTCTAGATCCTTCTCCAGAAATCATCGTCTTGAAAGAG | | |
| | CCTCCAACTATTCGACCCAATTCTCCCTATGACCTATGTAGCCGATTTGCAGCTGTCA | | |
| | TCGAGTCAATTCAGGGAGTTTCAACTGTCACAGTGAAATAAGCTCCCACATGTTCAAG | | |
| | GCCCATTCTGGTTCCTGGCTGCCTCCCTCTTGCACAGAAGTTCCTTGTCATACTGCTC | | |
| | ACCTTGGGAAAAGGATTAGGTGGGCACA | | |
| | ORF Start: ATG at 17 | | ORF Stop: TAA at 2939 |
| | SEQ ID NO:276 | 974 aa | MW at 110687.3 kD |
| NOV96b, | MTAELQQDDAAGAADGHGSSCQMLLNQLREITGIQDPSFLHEALKASNGDITQAVSLL | | |
| CG59708-02 Protein | | | |
| Sequence | TDERVKBPSQDTVATEPSEVEGSAANKEVLAKVTDLTHDNKDDLQAAIALSLLESPKI | | |
| | QADGRDLNRMHEATSAETKRSKRNIMFMQELQYLFALMMGSNRKFVDPSAALDLLKGA | | |
| | FRSSEEQQQDVSEFTHKLLDWLEDAFQLAVNVNSPRNKSENPMVQLFYGTFLTEGVRE | | |
| | GKPFCNNETFGQYPLQVNGYRNLDECLEGAMVEGDVELLPSDHSVKYGQERWFTKLPP | | |
| | VLTFELSRFEFNQSLGQPEKIHNKLEFPQIIYMDRYMYRSKELIRNKRECIRKLKEEI | | |
| | KILQQKLERYVKYGSGPARFPLPDMLKYVIEFASTKPASESCPPESDTHMTLPLSSVH | | |
| | CSVSDQTSKESTSTESSSQDVESTFSSPEDSLPKSKPLTSSRSSMEMPSQPAPRTVTD | | |
| | EEINFVKTCLQRWRSEIEQDIQDLKTCIASTTQTIEQMYCDPLLRQVPYRLHAVLVHE | | |
| | GQANAGHYWAYIYNQPRQSWLKYNDISVTESSWEEVERDSYGGLRNVSAYCLMYINAK | | |
| | LPYFNAEAAPTESDQMSEVEALSVELKHYIQEDNWRFEQEVEEWEEEQSCKIPQMESS | | |
| | PNSSSQGYSTSQEPSVASSHGVRCLSSEHAVIVKEQTAQAIANTARAYEKSGVEAALS | | |
| | EAFHEEYSRLYQLAKETPTSHSDPRLQHVLVYFFQNEAPKRVVERTLLEQFADKNLSY | | |
| | DERSISIMKVAQAKLKEIGPDDMNMEEYKRWHEDYSLFRKVSVYLLTGLELYQKGKYQ | | |
| | EALSYLVYAYQSNAALLMKGPRRGVKESVIALYRRKCLLELNAKAASLFETNDDHSVT | | |
| | EGINVMNELIIPCIHLIINNDISKDDLDAIEVMRNHWCSYLGQDIAENLQLCLGEFLP | | |
| | RLLDPSAEIIVLKEPPTIRPNSPYDLCSRFAAVMESIQGVSTVTVK | | |
| | SEQ ID NO:277 | 3231 bp | |
| NOV96c, | GCGCTTCGGCCATGACTGCGGAGCTGCAGCAGGACGACGCGGCCGGCGCGGCAGACGG | | |
| CG59708-03 DNA Sequence | | | |
| | CCACGGCTCGAGCTGCCAAATGCTGTTAAATCAACTGAGAGAAATCACAGGCATTCAG | | |
| | GACCCTTCCTTTCTCCATGAAGCTCTGAGGGCCAGTAATGGTGACATTACTCAGGCAG | | |
| | TCAGCCTTCTCACTGATGAGAGAGTTAAGGAGCCCAGTCAAGACACTGTTGCTACAGA | | |
| | ACCATCTGAAGTAGAGGGGAGTGCTGCCAACAAGGAAGTATTAGCAAAAGTTATAGAC | | |
| | ACCATCTGAAGTAGAGGGGAGTGCTGCCAACAAGGAAGTATTAGCAAAAGTTATAGAC | | |
| | CTTACTCATGATAACAAAGATGATCTTCAGGCTGCCATTGCTTTGAGTCTACTGGAGT | | |
| | CTCCCAAAATTCAAGCTGATGGAAGAGATCTTAACAGGATGCATGAAGCAACCTCTGC | | |
| | AGAAACTAAACGCTCAAAGAGAAAACGCTGTGAAGTCTGGGGAGAAAACCCCAATCCC | | |
| | AATGACTGGAGGAGAGTTGATGGTTGGCCAGTTGGGCTGAAAAATGTTGGCAATACAT | | |
| | GTTGGTTTAGTGCTGTTATTCAGTCTCTCTTTCAATTGCCTGAATTTCGAAGACTTGT | | |
| | TCTCAGTTATAGTCTGCCACAAAATGTACTTGAAAATTGTCGAAGTCATACAGAAAAG | | |
| | AGAAATATCATGTTTATGCAAGAGCTTCAGTATTTGTTTGCTCTAATGATGGGATCAA | | |
| | ATAGAAAATTTGTAGACCCGTCTGCAGCCCTGGATCTATTAAAGGGAGCATTCCGATC | | |
| | ATCTGAGGAACAGCAGCAAGATGTGAGTGAATTCACACACAAGCTCCTGGATTGGCTA | | |
| | GAGGACGCATTCCAGCTAGCTGTTAATGTTAACAGTCCCAGGAACAAATTTGAAAATC | | |
| | CAATGGTGCAGCTGTTCTATGGTACTTTCCTGACTGAAGGGGTTCGTGAAGGAAAACC | | |
| | CTTTTGTAACAATGAGACCTTCGCCCAGTATCCTCTTCAGCTAAACGGTTATCCCAAC | | |
| | TTAGACGAGTGTTTCGAAGGGGCCATGGTGGAGGCTGATGTTGAGCTTCTTCCCTCCG | | |
| | ATCACTCGGTGAAGTATGGACAAGAGCGTTGGTTTACAAAGCTACCTCCAGTGTTGAC | | |
| | CTTTGAACTCTCAACATTTGAGTTTAATCAGTCCCTTGGGCAGCCAGAGAAAATTCAC | | |
| | AATAAGCTGGAATTTCCTCAGATTATTThTATGGACAQGTACATGTACAGGAGCAAGG | | |
| | GCAGCAAAAATTGGAAGGGTATGTGAAATATGGCTCAGGCCCAGCTCGGTTCCCGCTC | | |
| | CCGGACATGCTCAAATATCTTATTGAATTTCCTAGTACAAAACCTGCCTCAGAAAGCT | | |
| | GTCCACCTGAAACTGACACACATATGACATTACCACTTTCTTCAGTGCACTGCTCGCT | | |
| | AGTACCTTTTCTTCTCCTGAAGATTCTTTACCCAAGTCTAAACCACTGACATCTTCTC | | |
| | GGTCTTCCATGGAAATGCCTTCACAGCCAGCTCCACGAACACTCACAGATGAGGAGAT | | |
| | AAATTTTGTTAAGACCTGTCTTCAGAGATGCAGCAGTGAGATTCAACAAGATATACAA | | |
| | GATTTAAAGACTTGTATTGCAAGTACTACTCAGACTATTCAACAGATGTACTGCGATC | | |
| | CTCTCCTTCGTCAGGTGCCTTATCGCTTGCATGCAGTTCTTGTTCATCAAGGACAAGC | | |
| | AAATGCTGGACACTATTGGGCCTATATCTATAATCAACCCCGACAGAGCTGCCTCAAG | | |
| | TACAATGACATCTCTCTTACTGAATCTTCCTGGGAACAAGTTGAAAGAGATTCCTATG | | |
| | CACCCCTGAGAAATGTTACTGCTTACTGTCTGATGTACATTAACGACAAACTACCCTA | | |
| | CTCATCACAGGACTACTCTACATCACAAGAGCCTTCAGTACCCTCTTCTCATGGGCTT | | |
| | CGCTGCTTCTCATCTGAGCATGCTGTGATTGTAAAGGAGCAAACTGCCCAGGCTATTG | | |
| | CAAACACAGCCCGTGCCTATGAGAAGAGCCGTGTACAAGCGGCACTCAGTGAGGCATT | | |
| | CCATGAACAATACTCCAGGCTCTATCAGCTTGCCAAAGAGACCCCCACCTCTCACAGT | | |
| | GATCCTCGACTTCAGCATGTCCTTGTCTACTTTTTCCAAAATGAAGCACCCAAAAGGG | | |
| | TAGTAGAACGAACCCTTCTGGAACAGTTTGCAGATAAAAATCTTAGCTATGATGAAAG | | |
| | ATGAATATGGAAGAGTACAAGAAGTGGCATGAAGATTATAGTTTGTTCCGAAAAGTGT | | |
| | CTGTGTATCTCCTAACAGGCCTAGAACTCTATCAAAAAGGAAAGTACCAAGAGGCACT | | |
| | TTCCTACCTGGTATATGCCTACCAGAGCAATGCTGCCCTGCTGATGAAGGGGCCCCCC | | |
| | CGGGGGGTCAAACAATCCGTGATTGCTTTATACCGAAGAAAATGCCTTCTGCAGCTGA | | |
| | ATGCCAAAGCAGCTTCTCTTTTTGAAACAAATGATGATCACTCCGTAACTGAGGGCAT | | |
| | TAATGTGATGAATGAACTGATCATCCCCTGCATTCACCTTATCATTAATAATGACATT | | |
| | GGCAAGATATTGCAGAAAATCTGCAGCTGTGCCTAGGGGAGTTTCTACCCAGACTTCT | | |
| | AGATCCTTCTGCAGAAATCATCCTCTTGAAAGAGCCTCCAACTATTCGACCCAATTCT | | |
| | CCCTATGACCTATGTAGCCGATTTCCAGCTGTCATGGAGTCAATTCAGGGAGTTTCAA | | |
| | CTGTGACACTGAAATAAGCTCCCACATGTTCAAGCCCCATTCTGGTTCCTGGCTGCCT | | |
| | GCCTCTTGCACAGAACTTCCTTGTCATAGTGCTCACCTTGG | | |
| | ORF Start: ATG at 12 | | ORF Stop: TAA at 3147 |
| | SEQ ID NO:278 | 1045 aa | MW at 119107.7 kD |

| | |
|---|---|
| NOV96c, | MTAELQQDDAAGAADGHGSSCQMLLNQLREITGIQDPSFLHEALKASNGDITQAVSLL |
| CG59708-03 Protein | |
| Sequence | TDERVKBPSQDTVATEPSEVEGSAANKEVLAKVTDLTHDNKDDLQAAIALSLLESPKI |
| | QADGRDLNRMHEATSAETKRSKRKRCEVWGENPNPNDWRRVDGWPVGLKNVGNTCWFS |
| | AVIQSLFQLPEFRRLVLSYSLPQNVLENCRSHTEKRNIMFMQELQYLFALMMGSNRKF |
| | VDPSAALDLLKGAFRSSEEQQQDVSEFTHKLLDWLEDAFQLAVNVNSPRNKSENPMVQ |
| | LFYGTFLTEGVREGKPFCNNETFGQYPLQVNGYRNLDECLEGAMVEGDVELLPSDHSV |
| | KYGQERWFTKLPPVLTFELSRFEFNQSLGQPEKIHNKLEFPQIIYMDRYMYRSKELIR |
| | NKRECIRKLKEEIKILQQKLERYVKYGSGPARFPLPDMLKYVIEFASTKPASESCPPE |
| | SDTHMTLPLSSVHCSVSDQTSKESTSTESSSQDVESTFSSPEDSLPKSKPLTSSRSSM |
| | EMPSQPAPRTVTDEEINFVKTCLQRWRSEIEQDIQDLKTCIASTTQTIEQMYCDPLLR |
| | QVPYRLHAVLVHEGQANAGHYWAYIYNQPRQSWLKYNDISVTESSWEEVERDSYGGLR |
| | NVSAYCLMYINAKLPYFNAEAAPTESDQMSEVEALSVELKHYIQEDNWRFEQEVEEWE |
| | EEQSCKIPQMESSPNSSSQGYSTSQEPSVASSHGVRCLSSEHAVIVKEQTAQAIANTA |
| | RAYEKSGVEAALSEAFHEEYSRLYQLAKETPTSHSDPRLQHVLVYFFQNEAPKRVVER |
| | TLLEQFADKNLSYDERSISIMKVAQAKLKEIGPDDMNMEEYKRWHEDYSLFRKVSVYL |
| | LTGLELYQKGKYQEALSYLVYAYQSNAALLMKGPRRGVKESVIALYRRKCLLELNAKA |
| | ASLFETNDDHSVTEGINVMNELIIPCIHLIINNDISKDDLDAIEVMRNHWCSYLGQDI |
| | AENLQLCLGEFLPRLLDPSAEIIVLKEPPTIRPNSPYDLCSRFAAVMESIQGVSTVTVK |

Sequence comparison of the above protein sequences yields the following sequence relationships shown in Table 96B.

TABLE 96B
Comparison of NOV96a against NOV96b through NOV96c.

| | | Identities/ |
|---|---|---|
| Protein | NOV96a Residues/ | Similarities for |
| Sequence | Match Residues | the Matched Region |
| NOV96b | 209 . . . 1045 | 805/837 (96%) |
| | 138 . . . 974 | 805/837 (96%) |
| NOV96c | 1 . . . 1045 | 979/1045 (93%) |
| | 1 . . . 1045 | 981/1045 (93%) |

Further analysis of the NOV96a protein yielded the following properties shown in Table 96C.

TABLE 96C
Protein Sequence Properties NOV96a

| | |
|---|---|
| PSort | 0.8800 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV96a protein against the Geneseq database, a proprietary database that contains sequences published in patents and patent publication, yielded several homologous proteins shown in Table 96D.

TABLE 96D
Geneseq Results for NOV96a

| | | NOV96a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE04874 | Human protease protein-1 (PRTS- | 22 . . . 1036 | 524/1035 (50%) | 0.0 |
| | 1) Homo sapiens, 1055 aa. | 18 . . . 1047 | 713/1035 (68%) | |
| | [WO200146443-A2, | | | |
| | 28 JUN. 2001] | | | |
| AAB31552 | A human ubiquitin specific | 22 . . . 1036 | 524/1035 (50%) | 0.0 |
| | protease 25 (USP25)-Homo | 18 . . . 1047 | 713/1035 (68%) | |
| | sapiens, 1055 aa. [WO200079267- | | | |
| | A2, 28 DEC. 2000] | | | |
| AAB31546 | A human ubiquitin specific | 22 . . . 1036 | 524/1035 (50%) | 0.0 |
| | protease 25 (USP25)-Homo | 18 . . . 1047 | 713/1035 (68%) | |
| | sapiens, 1055 aa. [WO200078934- | | | |
| | A2, 28 DEC. 2000] | | | |
| AAB74491 | Human SYK kinase binding | 22 . . . 1036 | 522/1035 (50%) | 0.0 |
| | protein sapiens, 1055 aa. | 18 . . . 1047 | 710/1035 (68%) | |
| | [WO200121654-A2, | | | |
| | 29 MAR. 2001] | | | |
| AAB31556 | A human ubiquitin specific | 22 . . . 1036 | 525/1067 (49%) | 0.0 |
| | protease (USP)-Homo sapiens, | 18 . . . 1079 | 717/1067 (66%) | |
| | 1087 aa. [WO200079267-A2, | | | |
| | 28 DEC. 2000] | | | |

In a BLAST search of public sequence databases, the NOV96a protein was found to have homology to the proteins shown in the BLASTP data in Table 96E.

TABLE 96E
Public BLASTP Results for NOV96a

| | | NOV96a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96RU2 | UBIQUITIN SPECIFIC PROTEASE- | 1 . . . 1045 | 1041/1077 (96%) | 0.0 |
| | Homo sapiens (Human), 1077 aa. | 1 . . . 1077 | 1042/1077 (96%) | |
| Q9P213 | KIAA1515 PROTEIN-Homo | 304 . . . 1045 | 738/742 (99%) | 0.0 |
| | sapiens (Human), 757 aa (fragment). | 16 . . . 757 | 739/742 (99%) | |
| P57080 | Ubiquitin carboxyl-terminal | 22 . . . 1036 | 527/1033 (51%) | 0.0 |
| | hydrolase 25 (EC 3.1.2.15) | 18 . . . 1047 | 710/1033 (68%) | |
| | (Ubiquitin thiolesterase 25) | | | |
| | (Ubiquitin-specific processing | | | |
| | protease 25) (Deubiquitinating | | | |
| | enzyme 25) (mUSP25)-Mus | | | |
| | musculus (Mouse), 1055 aa. | | | |
| Q9UHP3 | Ubiquitin carboxyl-terminal | 22 . . . 1036 | 525/1067 (49%) | 0.0 |
| | hydrolase 25 (EC 3.1.2.15) | 18 . . . 1079 | 717/1067 (66%) | |
| | (Ubiquitin thiolesterase 25) | | | |
| | (Ubiquitin-specific processing | | | |
| | protease 25) (Deubiquitinating | | | |
| | enzyme 25) (USP on chromosome | | | |
| | 21)-Homo sapiens (Human), 1087 | | | |
| | aa. | | | |
| Q9H9W1 | CDNA FLJ12512 FIS, CLONE | 313 . . . 1036 | 363/733 (49%) | 0.0 |
| | NT2RM2001730, WEAKLY | 2 . . . 729 | 510/733 (69%) | |
| | SIMILAR TO PROBABLE | | | |
| | UBIQUITIN CARBOXYL- | | | |
| | TERMINAL HYDROLASE | | | |
| | K02C4.3 (EC 3.1.2.15)-Homo | | | |
| | sapiens (Human), 737 aa. | | | |

PFam analysis predicts that the NOV96a protein contains the domains shown in the Table 96F.

TABLE 96F
Domain Analysis of NOV96a

| | | Identities/ | |
|---|---|---|---|
| Pfam | NOV96a | Similarities for | Expect |
| Domain | Match Region | the Matched Region | Value |
| UIM: domain 1 of 1 | 96 . . . 113 | 9/18 (50%) | 8.4 |
| | | 14/18 (78%) | |
| UCH-1: domain 1 of 1 | 162 . . . 193 | 14/32 (44%) | 2.6e−11 |
| | | 28/32 (88%) | |
| UCH-2: domain 1 of 1 | 580 . . . 649 | 26/72 (36%) | 1.5e−19 |
| | | 56/72 (78%) | |

### Example 97

The NOV97 clone was analyzed, and the nucleotide and predicted polypeptide sequences are shown in Table 97A.

TABLE 97A
NOV97 Sequence Analysis

| | SEQ ID NO:279 | 1601 bp | |
|---|---|---|---|
| NOV97a, | AGGGCAGAGGCCACAGCGCCATCCCCTTCCCCATGGTCTCCCTACCCCCAACCTGCAC | | |
| CG59559-01 DNA Sequence | | | |
| | TGGGCGCTCCGCCCAGAGGTGAGTCCCTCCCAGCCCTTCTCTCCTTCTGTCCTAGCCA | | |
| | TCCGCAGAGCCATCCTCTGCAAAGGAAGGAGCTAGGCTGTGCGCCCTGGGCCTCATGA | | |
| | GGGGGACTCTGTGCATAGGGCAGTATACAAGGACCTGGTGCTTCTGCTGCAGAAGGAC | | |
| | CGCCTGCTCACTCCCGGGCAGCTTAGAGCAAGGGGGCAGCTGAACTTCGAACAAGATG | | |
| | AGCTGGTGGACGGAGGCCAGCGGGGCCACATGCACAACGGCCTTAACTACCGTGAGGT | | |
| | CCGCGAGTTCCGCTCCGACCACCATCTGCTACGTTTTTACTTCCTCACCCGCGTGTAC | | |
| | TCCCATTACCTCCAGACCATCTTGAAAGAGCTGCAGTCGGGCGAGCACGCCCCCGACC | | |
| | TGGTCATCATGAATTCCTGCCTCTGGGACATCTCCAGGTATGGTCCGAACTCCTGGAG | | |
| | AAGCTACCTCGAGAACCTGGAGAACCTGTTCCAGTCCCTGGGCCAGGTGCTGCCCGAG | | |
| | TCTTGCCTCCTGGTGTGGAACACGGCCATGCCTGTGGGCGAGGAAGTCACCGGGGGTT | | |
| | TTCTTCCGCCCAAGCTCCGGCGGCACAAGGCCACCTTCCTGAAAAACGAAGTGGTCAA | | |
| | AGCCAACTTCCACAGCGCCACCGACGCACGTAAACATAACTTCGATGTACTGGACTTG | | |
| | CATTTCCACTTCCCCCACGCGAGGGAGAACCTGCACTGGGACGGGGTGCACTGGAATG | | |
| | GACGTGTGCACCGCTGCCTCTCCCAGCTGCTGCTGGCCCACGTGGCCGACGCCTGGGG | | |
| | TGTGGACCTGCCCCACCGCCACCCCGTGGGCGAGTGGATCAAGAAGAAAAAACCTGGC | | |
| | CCGAGAGTCGAAGGGCCGCCCCAGGCCAACAGAAATCACCCGGCCTTACCTCTGTCCC | | |
| | CACCCTTACCTTCCCCCACATACCGCCCCCTGCTTGGGTTCCCACCCCAGCGCTTGCC | | |
| | GCTGCTCCCGCTCCTGTCCCCACAGCCTCCTCCTCCCATTCTCCATCACCAGGGAATG | | |
| | CCCCGGTTCCCACACGGTCCCCCAGATGCCTGTTTTTCCTCAGACCATACTTTCCAGT | | |
| | CGCATCAATTCTATTGCCATTCAGATGTCCCCTCATCAGCCCATGCAGGTTTCTTCGT | | |
| | CGAAGACAATTTTATGGTTGGTCCTCAGCTGCCTATGCCCTTCTTCCCCACACCCCGT | | |
| | TATCAGCGGCCTGCCCCAGTGGTACATAGGGGTTTTGGCAGGTATCGTCCCCGTGGCC | | |
| | CCTATACGCCCTGGGGACAGCGGCCTCGACCTTCAAAGAGAAGGGCCCCAGCCAATCC | | |
| | TGAGCCAAGGCCTCAATAGACGGACCTAGGCCTTATTTCCTCTTTATGAACATGGATT | | |
| | GGACAGATCTGACACTTCCTTTCCATTGCTTCGCCTGAACAGACTGACCTTGTTAACT | | |
| | TAAGCCTCCAGTCCATGCCTCGTCTTCCTTTTGTT | | |
| | ORF Start: ATG at 171 | | ORF Stop: TAG at 1467 |
| | SEQ ID NO:280 | 432 aa | MW at 49726.6 kD |

| | |
|---|---|
| NOV97a, | MILLRASEVRQLLHNKFVVILGDSVHRAVYKDLVLLLQKDRLLTPGQLRARGELNFEQ |
| CG59559-01 Protein Sequence | |
| | DELVDGGQRGHMHNGLNYREVREFRSDHHLVRFYFLTRVYSDYLQTILKELQSGEHAP |
| | DLVIMNSCLWDISRYGPNSWRSYLENLENLFQCLGQVLPESCLLVWNTAMPVGEEVTG |
| | GFLPPKLRRQKATFLKNEVVKANFHSATEARKHNFDVLDLHFHFRHARENLHWDGVHW |
| | NGRVHRCLSQLLLAHVADAWGVELPHRHPVGEWIKKKKPGPRVEGPPQANRNHPALPL |
| | SPPLPSPTYRPLLGFPPQRLPLLPLLSPQPPPPILHHQGMPRFPQGPPDACFSSDHTF |
| | QSDQFYCHSDVPSSAHAGFFVEDNFMVGPQLPMPFFPTPRYQRPAPVVHRGFGRYRPR |
| | GPYTPWGQRPRPSKRRAPANPEPRPQ |

Further analysis of the NOV97a protein yielded the following properties shown in Table 97B.

TABLE 97B
Protein Sequence Properties NOV97a

| | |
|---|---|
| PSort | 0.5937 probability located in mitochondrial matrix space; |
| analysis: | 0.5103 probability located in microbody (peroxisome); 0.4900 |
| | probability located in nucleus; 0.3252 probability located in |
| | lysosome (lumen) |
| SignalP | No Known Signal Sequence Predicted |
| analysis: | |

A search of the NOV97a protein against the Geneseq database, a proprietary database that contains sequences published in patents and patent publication, yielded several homologous proteins shown in Table 97C.

TABLE 97C
Geneseq Results for NOV97a

| | | NOV97a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG74241 | Human colon cancer antigen protein | 34 . . . 294 | 162/268 (60%) | 1e−82 |
| | SEQ ID NO: 5005-Homo sapiens, | 1 . . . 266 | 191/268 (70%) | |
| | 281 aa. [WO200122920-A2, | | | |
| | 5 APR. 2001] | | | |
| AAE03639 | Human extracellular matrix and cell | 1 . . . 421 | 197/435 (45%) | 2e−82 |
| | adhesion molecule-3 (XMAD-3)- | 1 . . . 366 | 231/435 (52%) | |
| | Homo sapiens, 386 aa. | | | |
| | [WO200142285-A2, 14 JUN. 2001] | | | |

In a BLAST search of public sequence databases, the NOV97a protein was found to have homology to the proteins shown in the BLASTP data in Table 97D.

TABLE 97D
Public BLASTP Results for NOV97a

| | | NOV97a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96HM7 | SIMILAR TO HYPOTHETICAL | 1 . . . 432 | 432/432 (100%) | 0.0 |
| | PROTEIN FLJ22376-Homo sapiens | 1 . . . 432 | 432/432 (100%) | |
| | (Human), 432 aa. | | | |
| Q96B20 | HYPOTHETICAL 31.4 KDA | 121 . . . 310 | 190/190 (100%) | e−116 |
| | PROTEIN-Homo sapiens (Human), | 1 . . . 190 | 190/190 (100%) | |
| | 279 aa. | | | |
| Q9H1Q7 | BA12M19.1.3 (NOVEL PROTEIN) | 1 . . . 421 | 234/437 (53%) | e−111 |
| | (CDNA FLJ31791 FIS, CLONE | 18 . . . 434 | 273/437 (61%) | |
| | NT2RI2008749, WEAKLY | | | |
| | SIMILAR TO SPLICEOSOME | | | |
| | ASSOCIATED PROTEIN 49)- | | | |
| | Homo sapiens (Human), 454 aa. | | | |
| Q9H1Q6 | BA12M19.1.1 (NOVEL PROTEIN)- | 1 . . . 421 | 197/435 (45%) | 7e−82 |
| | Homo sapiens (Human), 403 aa. | 18 . . . 383 | 231/435 (52%) | |
| Q9H6D1 | CDNA: FLJ22376 FIS, CLONE | 1 . . . 421 | 196/435 (45%) | 1e−81 |
| | HRC07327-Homo sapiens | 18 . . . 383 | 231/435 (53%) | |
| | (Human), 403 aa. | | | |

PFam analysis predicts that the NOV97a protein contains the domains shown in the Table 97E.

TABLE 97E
Domain Analysis of NOV97a

| | | | |
|---|---|---|---|
| | | Identities/ | |
| Pfam | NOV97a | Similarities for | Expect |
| Domain | Match Region | the Matched Region | Value |

No Significant Matches Found

The NOV98 clone was analyzed, and the nucleotide and predicted polypeptide sequences are shown in Table 98A.

TABLE 98A
NOV98 Sequence Analysis

| | SEQ ID NO:281 | 981 bp | |
|---|---|---|---|
| NOV98a, | GCGCCGGGTCCCAGAATCTAGTCCTACGCCACGGTTTTGACCACGCGTGACCCGCTGC | | |
| CG59669-01 DNA Sequence | | | |
| | CCAGCCGGCCCGGCCATCAGGTGGTCCGTGTGTCCCTCTGACATGTCGTCCTGCAGCC | | |
| | GCGTGGCCCTGGTAACTGGGGCTAACAAAGGCATCGGCTTTGCGATCACGCGTGACCT | | |
| | GTGTCGGAAATTCTCCGGGGACGTGGTGCTCACGGCGCGGGACGAGGCGCGGGGCCGC | | |
| | GCGGCGGTGCAGCAGCTGCAGGCGGAGGGCCTGAGCCCACGCTTCCACCAGCTGGACA | | |
| | TCGACGACCCGCAGAGCATCCGTGCGCTGCGCGACTTTCTGCGCAAGGAGTACGGGGG | | |
| | ACTTAACGTGCTGGTCAACAACGCGGGCATCGCCTTTAGAAGTACTGATCTCACCCAC | | |
| | TTTCACATTCTAAGAGAAGCTGCAATGAAAACTAACTTTTTTGGTACCCAGGCCGTCT | | |
| | GCACAGAGCTACTCCCTCTAATAAAAACCCAAGGTAGAGTGGTGAATATATCAAGCCT | | |
| | AATAAGTCTAGAGGCCCTGAAAAACTGCAGCCTGGAGCTACAGCAGAAGTTTCGAAGT | | |
| | GAGACCATCACAGAGGAGGAGCTGGTGGGGCTCATGAACAAGTTTGTGGAGGATACAA | | |
| | AGAAAGGAGTCCATGCAAAAGAAGGCTGGCCTAATAGTGCATACGGGGTGTCTAAGAT | | |
| | TGGAGTGACAGTCCTGTCCAGAATCCTTGCCAGGAAACTCAATGAGCAGAGGAGAGGG | | |
| | GACAAGATCCTTCTGAATGCCTGCTGCCCTGGCTGGGTCAGAACCGACATGGCAGGAC | | |
| | CACAAGCCACCAAAAGCCCAGAAGAAGGAGCAGAGACCCCTGTGTACTTGGCCCTTTT | | |
| | GCCTCCAGATGCAGAGGGACCTCATGGGCAGTTTGTTCAAGATAAAAAAGTGGAACAA | | |
| | TGGTGAACTCAGCTCTTTGTACAGCTCCCATCTGTAGCCTGTCCTAAAGGGGA | | |
| | ORF Start: ATG at 101 | | ORF Stop: TGA at 932 |
| | SEQ ID NO:282 | 277 aa | MW at 30547.7 kD |

| | |
|---|---|
| NOV98a, | MSSCSRVALVTGANKGIGFAITRDLCRKFSGDVVLTARDEARGRAAVQQLQAEGLSPR |
| CG59669-01 Protein Sequence | |
| | FHQLDIDDPQSIRALRDFLRKEYGGLNVLVNNAGIAFRSTDLTHFHILREAAMKTNFF |
| | GTQAVCTELLPLIKTQGRVVNISSLISLEALKNCSLELQQKFRSETITEEELVGLMNK |
| | FVEDTKKGVHAKEGWPNSAYGVSKIGVTVLSRILARKLNEQRRGDKILLNACCPGWVR |
| | TDMAGPQATKSPEEGAETPVYLALLPPDAEGPHGQFVQDKKVEQW |

Further analysis of the NOV98a protein yielded the following properties shown in Table 98B.

TABLE 98B
Protein Sequence Properties NOV98a

| | |
|---|---|
| PSort | 0.4766 probability located in mitochondrial matrix space; |
| analysis: | 0.4500 probability located in cytoplasm; 0.1822 probability |
| | located in mitochondrial inner membrane; 0.1822 probability |
| | located in mitochondrial intermembrane space |
| SignalP | No Known Signal Sequence Predicted |
| analysis: | |

A search of the NOV98a protein against the Geneseq database, a proprietary database that contains sequences published in patents and patent publication, yielded several homologous proteins shown in Table 98C.

TABLE 98C
Geneseq Results for NOV98a

| | | NOV98a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAW51011 | Human liver carbonyl reductase- | 1 . . . 277 | 236/277 (85%) | e−134 |
| | Homo sapiens, 277 aa. | 1 . . . 277 | 252/277 (90%) | |
| | [U.S. Pat. No. 5756299-A, | | | |
| | 26 MAY 1998] | | | |
| AAU33100 | Novel human secreted protein | 142 . . . 277 | 119/136 (87%) | 2e−66 |
| | #3591-Homo sapiens, 175 aa. | 39 . . . 174 | 128/136 (93%) | |
| | [WO200179449-A2, 25 Oct. 2001] | | | |
| AAM73641 | Human bone marrow expressed | 1 . . . 97 | 86/97 (88%) | 7e−43 |
| | probe encoded protein SEQ ID NO: | 1 . . . 97 | 92/97 (94%) | |
| | 33947-Homo sapiens, 123 aa. | | | |
| | [WO200157276-A2, 9 AUG. 2001] | | | |
| AAM60948 | Human brain expressed single exon | 1 . . . 97 | 86/97 (88%) | 7e−43 |
| | probe encoded protein SEQ ID NO: | 1 . . . 97 | 92/97 (94%) | |
| | 33053-Homo sapiens, 123 aa. | | | |
| | [WO200157275-A2, 9 AUG. 2001] | | | |
| AAM33832 | Peptide #7869 encoded by probe for | 1 . . . 97 | 86/97 (88%) | 7e−43 |
| | measuring placental gene expression- | 1 . . . 97 | 92/97 (94%) | |
| | Homo sapiens, 123 aa. | | | |
| | [WO200157272-A2, 9 AUG. 2001] | | | |

In a BLAST search of public sequence databases, the NOV98a protein was found to have homology to the proteins shown in the BLASTP data in Table 98D.

TABLE 98D
Public BLASTP Results for NOV98a

| | | NOV98a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q924V2 | CARBONYL REDUCTASE 2- | 1 . . . 277 | 243/277 (87%) | e−139 |
| | Cricetulus griseus (Chinese | 1 . . . 277 | 260/277 (93%) | |
| | hamster), 277 aa. | | | |
| Q91X28 | SIMILAR TO CARBONYL | 1 . . . 277 | 244/277 (88%) | e−139 |
| | REDUCTASE 1-Mus musculus | 1 . . . 277 | 256/277 (92%) | |
| | (Mouse), 277 aa. | | | |
| Q924V3 | CARBONYL REDUCTASE 1- | 1 . . . 277 | 241/277 (87%) | e−137 |
| | Cricetulus griseus (Chinese | 1 . . . 277 | 256/277 (92%) | |
| | hamster), 277 aa. | | | |
| P48758 | Carbonyl reductase [NADPH] 1 (EC | 2 . . . 277 | 240/276 (86%) | e−136 |
| | 1.1.1.184) (NADPH-dependent | 1 . . . 276 | 253/276 (90%) | |
| | carbonyl reductase 1)-Mus | | | |
| | musculus (Mouse), 276 aa. | | | |
| JC5284 | carbonyl reductase (NADPH) (EC | 1 . . . 277 | 236/277 (85%) | e−134 |
| | 1.1.1.184), inducible-rat, 277 aa. | 1 . . . 277 | 249/277 (89%) | |

PFam analysis predicts that the NOV98a protein contains the domains shown in the Table 98E.

TABLE 98E
Domain Analysis of NOV98a

| | | Identities/ | |
|---|---|---|---|
| Pfam | NOV98a | Similarities for | Expect |
| Domain | Match Region | the Matched Region | Value |
| adh_short: domain 1 | 4 . . . 274 | 67/286 (23%) | 1.6e−38 |
| of 1 | | 185/286 (65%) | |

### Example 99.

The NOV99 clone was analyzed, and the nucleotide and predicted polypeptide sequences are shown in Table 99A.

TABLE 99A
NOV99 Sequence Analysis

| | SEQ ID NO:283 | 1001 bp | |
|---|---|---|---|
| NOV99a, | CTTGGTATAAGTAAGTGCTCGTCAATGTTGGCTACTCTCAATGTCAGAGCCGCAGCCG | | |
| CG58624-01 DNA Sequence | | | |
| | CGGGGCGCAGAGCGCGATCTCTACCGGGACACGTGGGTGCGATACCTGGGCTATGCCA | | |
| | ATGAGGTGGGCGAGGCTTTCCGCTCTCTTGTGCCAGCGGCGGTGGTGTGGCTGAGCTA | | |
| | TGGCGTGGCCAGCTCCTACGTGCTGGCGGATGCCATTGACAAAGGCAAGAAGGCTGGA | | |
| | GAGGTGCCCAGCCCTGAAGCAGGCCGCAGCGCCAGGGTGACTGTGGCTGTGGTGGACA | | |
| | CCTTTGTATGGCAGGCTCTAGCCTCTGTGGCCATTCCGGGCTTCACCATCAACCGCGT | | |
| | GTGTGCTGCCTCTCTCTATGTCCTGGGCACTGCCACCCGCTGGCCCCTGGCTGTCCGC | | |
| | AAGTGGACCACCACCGCGCTTGGGCTGTTGACCATCCCCATCATTATCCACCCCATTG | | |
| | ACAGGGATCATCCACTCTCCAGTGATGAGAGTGGATCATCCAGTCTCCAGCACGAAGG | | |
| | GCCAGGGGTCCCACAGGTGAGTGGAGCCCCAGCAGCCCCCTCAGCTCTGCGTGCCCAT | | |
| | GTACTGGTCTTCTCCCTGGCTCTATACTCAGTGTTCAAGGGGTTGGACGGGGCTTGGG | | |
| | CCGCGGAGCTGCGCCTGGCTTTGCTGCTCCACAAGGGCACCGTGGCTGTCAGCCTGTC | | |
| | CCTGCAACTGCTGCAGAGCCACGTAGGGTTACAGGTGGTGGCTGGCTGTGGGATCCAC | | |
| | TTCTTGTGCATGACACTTCTAGGCATCCGGCTGGGTGCGGCTCTGGCACAGTCAGCAG | | |
| | GGCCTCTGCACCAGCTGGCCCAGTCTGTGCTAGAGGGCATGGTGGCTGGCACCTTCCT | | |
| | GTATACCACCTTTCTGGAAATCTTTCCACAGGAGCTGGCGACTTCTGAGCAAAGGATC | | |
| | CTCAAGGTCATTCTGCTCCTAGAAGGGTGTGCCCTGCTCACTGGCCTGCTCTTCATCC | | |
| | ATATCTAGGGGGCTT | | |
| | ORF Start: ATG at 41 | | ORF Stop: TAG at 992 |
| | SEQ ID NO:284 | 317aa | MW at 33737.8 kD |

| | |
|---|---|
| NOV99a, | MSEPQPRGAERDLYRDTWVRYLGYANEVGEAFRSLVPAAVVWLSYGVASSYVLADAID |
| CG58624-01 Protein Sequence | |
| | KGKKAGEVPSPEAGRSARVTVAVVDTFVWQALASVAIPGFTINRVCAASLYVLGTATR |
| | WPLAVRKWTTTALGLLTIPIIIHPIDRDHPLSSDESGSSSLQHEGPGVPQVSGAPAAP |
| | SALRAHVLVFSLALYSVFKGLDGAWAAELRLALLLHKGTVAVSLSLQLLQSHVGLQVV |
| | AGCGIHFLCMTLLGIRLGAALAQSAGPLHQLAQSVLEGMVAGTFLYTTFLEIFPQELA |
| | TSEQRILKVILLLEGCALLTGLLFIHI |

Further analysis of the NOV99a protein yielded the following properties shown in Table 99B.

TABLE 99B
Protein Sequence Properties NOV99a

| | |
|---|---|
| PSort | 0.6000 probability located in plasma membrane; |
| analysis: | 0.4000 probability located in Golgi body; 0.3000 |
| | probability located in endoplasmic reticulum (membrane); |
| | 0.1000 probability located in mitochondrial inner membrane |
| SignalP | Likely cleavage site between residues 55 and 56 |
| analysis: | |

A search of the NOV99a protein against the Geneseq database, a proprietary database that contains sequences published in patents and patent publication, yielded several homologous proteins shown in Table 99C.

TABLE 99C
Geneseq Results for NOV99a

| | | NOV99a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM93835 | Human polypeptide, SEQ ID NO: | 140 . . . 317 | 134/184 (72%) | 3e−63 |
| | 3905-Homo sapiens, 324 aa. | 141 . . . 324 | 145/184 (77%) | |
| | [EP1130094-A2, 5 SEP. 2001] | | | |
| AAY52394 | Human transmembrane protein | 140 . . . 317 | 134/184 (72%) | 3e−63 |
| | HP10528-Homo sapiens, 324 aa. | 141 . . . 324 | 145/184 (77%) | |
| | [WO9955862-A2, 4 NOV. 1999] | | | |
| AAY84895 | A human proliferation and | 140 . . . 317 | 134/184 (72%) | 3e−63 |
| | apoptosis related protein-Homo | 141 . . . 324 | 145/184 (77%) | |
| | sapiens, 324 aa. [WO200023589- | | | |
| | A2, 27 APR. 2000] | | | |
| AAB43291 | Human ORFX ORF3055 | 140 . . . 317 | 134/184 (72%) | 3e−63 |
| | polypeptide sequence SEQ ID | 140 . . . 323 | 145/184 (77%) | |
| | NO: 6110-Homo sapiens, 323 aa. | | | |
| | [WO200058473-A2, 5 OCT. 2000] | | | |
| AAM93650 | Human polypeptide, SEQ ID NO: | 140 . . . 317 | 133/184 (72%) | 2e−62 |
| | 3514-Homo sapiens, 324 aa. | 141 . . . 324 | 144/184 (77%) | |
| | [EP1130094-A2, 5 SEP. 2001] | | | |

In a BLAST search of public sequence databases, the NOV99a protein was found to have homology to the proteins shown in the BLASTP data in Table 99D.

TABLE 99D
Public BLASTP Results for NOV99a

| | | NOV99a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9UDX5 | WUGSC:H_DJ0539M06.2 PROTEIN - | 1 . . . 152 | 145/152 (95%) | 6e−78 |
| | Homo sapiens (Human), 166 aa. | 1 . . . 152 | 145/152 (95%) | |
| Q9CRB8 | 2610507A21RIK PROTEIN | 1 . . . 168 | 133/168 (79%) | 8e−69 |
| | (1700020C11RIK PROTEIN) - Mus | 1 . . . 164 | 143/168 (84%) | |
| | musculus (Mouse), 166 aa. | | | |
| Q9CZX4 | 2610507A21RIK PROTEIN - Mus | 1 . . . 143 | 125/143 (87%) | 2e−68 |
| | musculus (Mouse), 166 aa. | 1 . . . 143 | 133/143 (92%) | |
| Q9NY26 | IRT1 PROTEIN (SIMILAR TO | 140 . . . 317 | 134/184 (72%) | 1e−62 |
| | ZINC/IRON REGULATED | 141 . . . 324 | 145/184 (77%) | |
| | TRANSPORTER-LIKE) | | | |
| | (HYPOTHETICAL 34.2 KDA | | | |
| | PROTEIN) (UNKNOWN) (PROTEIN | | | |
| | FOR MGC:14180) - Homo sapiens | | | |
| | (Human), 324 aa. | | | |
| Q9Y380 | CGI-71 PROTEIN - Homo sapiens | 140 . . . 317 | 134/184 (72%) | 1e−62 |
| | (Human), 324 aa. | 141 . . . 324 | 145/184 (77%) | |

PFam analysis predicts that the NOV99a protein contains the domains shown in the Table 99E.

TABLE 99E
Domain Analysis of NOV99a

| | | Identities/ | | |
|---|---|---|---|---|
| | | Similarities | | |
| | NOV99a Match | for the | | Expect |
| Pfam Domain | Region | Matched Region | | Value |
| Syndecan: domain 1 of 1 | 235 . . . 255 | 9/21 | (43%) | 6.9 |
| | | 16/21 | (76%) | |
| Zip: domain 1 of 1 | 174 . . . 313 | 52/178 | (29%) | 2.3e−15 |
| | | 108/178 | (61%) | |

### Example 100

The NOV100 clone was analyzed, and the nucleotide and predicted polypeptide sequences are shown in Table 100A.

TABLE 100A
NOV100 Sequence Analysis

| | SEQ ID NO:285 | 987 bp | |
|---|---|---|---|
| NOV100a, | AGACGCTCACACAGACAACCTCAAGTCCAGCAACATCTTAGTAGCCCAAAATCGACTG | | |
| CG59679-01 DNA Sequence | | | |
| | CTTTAGTTCTTCTGGTGGGTGCCTCTCACTGTCCACTCGGCTATGCCATCCTGCAGTC | | |
| | GCATTGCACTGGTGACTGGAGCTAATAAGGGCATTGGCTTTGCGATCACTCGTGACCT | | |
| | GTGTCAGCAATTCTCAGGGGATGTGGTGCTCACTGCACGGGACGAGGCACGGGGCCTT | | |
| | GCGGCAGTGCAGAAGCTGCAGGCTGAGGGCCTGATTCCTCGCTTCCACCAGCTGGACA | | |
| | TCAATGACCCTCAGAGCATCCATGCACTTCGCAACTTTCTGCTCAAGGAGTACGGAGG | | |
| | CCTGGATGTGCTGGTCAACAACGCGGGCATTGGCGTGCTTTTCAAAGTGGATGACCCA | | |
| | ACACCCTTCGACATTCAAGCTGAGGTGACACTGAAGACGAACTTTTTTGCCACTAGAA | | |
| | ATGTCTGCACTGAGTTACTGCCTATAATGAAACCACATGGTAGAGTGGTGAACATCAG | | |
| | CAGTCTGCAGGGGTTAAAAGCCCTTGAGAACTGCAGGGAAGATCTTCAGGAAAAGTTC | | |
| | CGATGTGACACACTTACCGAGGTGGACCTGGTCGACCTCATGAAAAAGTTTGTGGAGG | | |
| | ATACAAAAAATGAAGTCCATGAGAGGGAAGGTTGGCCAGACTCGGCTTACGGGGTGTC | | |
| | GAAGCTGGGGGTGACAGTCCTTACGAGGATCCTGGCCCGGCAGCTGGATGAAAAGAGG | | |
| | AAAGCGGACAGGATTCTGCTCAATGCCTGCTGCCCGGGATGGGTGAAGACCGACATGG | | |
| | CGAGGGACCAGGGCTCCCGGACCGTGGAAGAGGGGGCCGAAACCCCCGTTTACTTGGC | | |
| | TCTCCTGCCTCCAGATGCCACTGAACCTCACGGCCAGCTAGTCCGTGACAAAGTTGTG | | |
| | CAAACTTGGTGAACGTCTGCTCTGGGGCTTAATTGTTTGATAAACGTTAGCGGGAGAGA | | |
| | ORF Start: ATG at 101 | | ORF Stop: TGA at 938 |
| | SEQ ID NO:286 | 279 aa | MW at 31007.2 kD |

| | |
|---|---|
| NOV100a, | MPSCSRIALVTGANKGIGFAITRDLCQQFSGDVVLTARDEARGLAAVQKLQAEGLIPR |
| CG59679-01 Protein Sequence | |
| | FHQLDINDPQSIHALRNFLLKEYGGLDVLVNNAGIGVLFKVDDPTPFDIQAEVTLKTN |
| | FFATRNVCTELLPIMKPHGRVVNISSLQGLKALENCREDLQEKFRCDTLTEVDLVDLM |
| | KKFVEDTKNEVHEREGWPDSAYGVSKLGVTVLTRILARQLDEKRKADRILLNACCPGW |
| | VKTDMARDQGSRTVEEGAETPVYLALLPPDATEPHGQLVRDKVVQTW |

Further analysis of the NOV100a protein yielded the following properties shown in Table 100B.

TABLE 100B
Protein Sequence Properties NOV100a

| | |
|---|---|
| PSort | 0.3600 probability located in mitochondrial matrix space; |
| analysis: | 0.3000 probability located in microbody (peroxisome); 0.1808 |
| | probability located in lysosome (lumen); 0.0000 probability |
| | located in endoplasmic reticulum (membrane) |
| SignalP | No Known Signal Sequence Predicted |
| analysis: | |

A search of the NOV100a protein against the Geneseq database, a proprietary database that contains sequences published in patents and patent publication, yielded several homologous proteins shown in Table 100C.

TABLE 100C
Geneseq Results for NOV100a

| | | NOV100a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAW51011 | Human liver carbonyl reductase - | 1 . . . 279 | 198/279 | (70%) | e−112 |
| | Homo sapiens, 277 aa. | 1 . . . 277 | 233/279 | (82%) | |
| | [US5756299-A, 26 MAY 1998] | | | | |
| AAU33100 | Novel human secreted protein | 145 . . . 279 | 88/135 | (65%) | 2e−48 |
| | #3591 - Homo sapiens, 175 aa. | 40 . . . 174 | 110/135 | (81%) | |
| | [WO200179449-A2, 25 OCT | | | | |
| | 2001] | | | | |
| AAG46601 | Arabidopsis thaliana protein | 3 . . . 259 | 106/268 | (39%) | 6e−43 |
| | fragment SEQ ID NO: 58644 - | 20 . . . 283 | 157/268 | (58%) | |
| | Arabidopsis thaliana, 302 aa. | | | | |
| | [EP1033405-A2, 6 SEP 2000] | | | | |
| AAG46600 | Arabidopsis thaliana protein | 3 . . . 259 | 106/268 | (39%) | 6e−43 |
| | fragment SEQ ID NO: 58643 - | 34 . . . 297 | 157/268 | (58%) | |
| | Arabidopsis thaliana, 316 aa. | | | | |
| | [EP1033405-A2, 6 SEP 2000] | | | | |
| AAG46599 | Arabidopsis thaliana protein | 3 . . . 259 | 106/268 | (39%) | |
| | fragment SEQ ID NO: 58642 - | 45 . . . 308 | 157/268 | (58%) | |
| | Arabidopsis thaliana, 327 aa. | | | | |
| | [EP1033405-A2, 6 SEP 2000] | | | | |

In a BLAST search of public sequence databases, the NOV100a protein was found to have homology to the proteins shown in the BLASTP data in Table 100D.

TABLE 100D
Public BLASTP Results for NOV100a

| | | NOV100a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9JJN7 | CARBONYL REDUCTASE (EC | 1 . . . 279 | 246/279 (88%) | e−140 |
| | 1.1.1.184) (CARBONYL | 1 . . . 277 | 262/279 (93%) | |
| | REDUCTASE 3) - Cricetulus | | | |
| | griseus (Chinese hamster), 277 aa. | | | |
| AAH02812 | CARBONYL REDUCTASE 3 - | 1 . . . 279 | 227/279 (81%) | e−126 |
| | Homo sapiens (Human), 277 aa. | 1 . . . 277 | 246/279 (87%) | |
| O75828 | Carbonyl reductase [NADPH] 3 | 3 . . . 279 | 226/277 (81%) | e−126 |
| | (EC 1.1.1.184) (NADPH- | 2 . . . 276 | 245/277 (87%) | |
| | dependent carbonyl reductase 3) - | | | |
| | Homo sapiens (Human), 276 aa. | | | |
| Q924V2 | CARBONYL REDUCTASE 2 - | 1 . . . 279 | 206/279 (73%) | e−119 |
| | Cricetulus griseus (Chinese | 1 . . . 277 | 244/279 (86%) | |
| | hamster), 277 aa. | | | |
| Q91X28 | SIMILAR TO CARBONYL | 1 . . . 279 | 204/279 (73%) | e−116 |
| | REDUCTASE 1 - Mus musculus | 1 . . . 277 | 240/279 (85%) | |
| | (Mouse), 277 aa. | | | |

PFam analysis predicts that the NOV100a protein contains the domains shown in the Table 100E.

TABLE 100E
Domain Analysis of NOV100a

| | | Identities/ | | |
|---|---|---|---|---|
| | | Similarities | | |
| | NOV100a Match | for the | | Expect |
| Pfam Domain | Region | Matched Region | | Value |
| adh_short: | 4 . . . 277 | 77/316 | (24%) | 5.2e−31 |
| domain 1 of 1 | | 186/316 | (59%) | |

### Example 101

The NOV101 clone was analyzed, and the nucleotide and predicted polypeptide sequences are shown in Table 101A.

TABLE 101A
NOV101 Sequence Analysis

| | SEQ ID NO:287 | 1011 bp | |
|---|---|---|---|
| NOV101a, | CTCCTCGGGGGGGCGGCGGCGGCGATGTTCTCGGTCCTCTCGTACGGGCGGCTGGTGG | | |
| CG59644-01 DNA Sequence | | | |
| | CCCGCGCCGTGCTCGGCGGCCTCTCGCAGACCGACCCCAGGGCCGGCGGCGGCGGCGG | | |
| | CGGCGCCGTGCTCGGCGGCCTCTCGCAGACCGACCCCAGGGCCGGCGGCGGCGGCGGC | | |
| | GGCGACTACGGACTGGTGACGGCCGGCTGCGGCTTCGGGAAGGACTTCCGTAAGGGCC | | |
| | TCCTCAAGAAGGGCGCGTGCTACGGGGACGACGCGTGCTTCGTGGCCCGGCACCGTTC | | |
| | CGCGGACGTGCTCGGTGTTGCAGATGGTGTAGGAGGCTGGAGAGACTATGGAGTTGAT | | |
| | CCATCTCAATTCTCAGGGACTTTAATGCGGACGTGTGAACGTTTAGTAAAAGAAGGAC | | |
| | GGTTCGTACCTAGTAATCCCATTGGAATTCTCACCACAAGCTACTGTGAGTTGCTGCA | | |
| | AAATAAAGTCCCTTTGCTCGGTAGCAGCACCGCCTGCATTGTGGTGCTGGACAGAACC | | |
| | AGCCACCGCTTACACACAGCAAACCTGGGCGATTCAGGCTTCCTGGTTGTCAGGGGTG | | |
| | GTGAAGTCGTGCACCGATCAGATGAGCAGCAGCATTACTTCAACACTCCATTCCAGCT | | |
| | CTCAATCGCTCCCCCTGAAGCCGAGGGAGTCGTCTTGAGCGACAGTCCGGATGCTGCT | | |
| | GATAGCACGTCTTTCGATGTCCAGCTAGGAGACATTATCCTGACGGCAACAGATGGAC | | |
| | TCTTTGACAACATGCCTGATTATATGATTCTTCAGGAGCTAAAAAAGTTAAAGAATTC | | |
| | AAATTATGAGAGTATACAACAGACTGCCAGAAGCATTGCTGAGCAAGCTCATGAGCTG | | |
| | GCCTATGACCCAAATTATATGTCACCTTTTGCACAGTTTGCATGTGACAATGGATTGA | | |
| | ATGTGAGAGGTGGTGGAAAGCCAGATGACATCACCGTCCTTCTTTCAATAGTGGCTGA | | |
| | GTATACAGACTAGCTGAGGTGTCAA | | |
| | ORF Start: ATG at 25 | | ORF Stop: TAG at 997 |
| | SEQ ID NO:288 | 324 aa | MW at 34311.1 kD |

| | |
|---|---|
| NOV101a, | MFSVLSYGRLVARAVLGGLSQTDPRAGGGGGGAVLGGLSQTDPRAGGGGGGDYGLVTA |
| CG59644-01 Protein Sequence | |
| | GCGFGKDFRKGLLKKGACYGDDACFVARHRSADVLGVADGVGGWRDYGVDPSQFSGTL |
| | MRTCERLVKEGRFVPSNPIGILTTSYCELLQNKVPLLGSSTACIVVLDRTSHRLHTAN |
| | LGDSGFLVVRGGEVVHRSDEQQHYFNTPFQLSIAPPEAEGVVLSDSPDAADSTSFDVQ |
| | LGDIILTATDGLFDNMPDYMILQELKKLKNSNYESIQQTARSIAEQAHELAYDPNYMS |
| | PFAQFACDNGLNVRGGGKPDDITVLLSIVAEYTD |

Further analysis of the NOV101a protein yielded the following properties shown in Table 101B.

TABLE 101B
Protein Sequence Properties NOV101a

| | |
|---|---|
| PSort | 0.5708 probability located in mitochondrial matrix space; |
| analysis: | 0.4996 probability located in mitochondrial intermembrane |
| | space; 0.2852 probability located in mitochondrial inner |
| | membrane; 0.2852 probability located in mitochondrial |
| | outer membrane |
| SignalP | Likely cleavage site between residues 23 and 24 |
| analysis: | |

A search of the NOV101a protein against the Geneseq database, a proprietary database that contains sequences published in patents and patent publication, yielded several homologous proteins shown in Table 101C.

TABLE 101C
Geneseq Results for NOV101a

| | | NOV101a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB85357 | Human phosphatase (PP) (clone ID | 1 . . . 324 | 304/324 (93%) | e−173 |
| | 3402521CD1) - Homo sapiens, 304 | 1 . . . 304 | 304/324 (93%) | |
| | aa. [WO200153469-A2, 26 JUL | | | |
| | 2001] | | | |
| AAU32112 | Novel human secreted protein | 25 . . . 324 | 272/300 (90%) | e−156 |
| | #2603 - Homo sapiens, 304 aa. | 6 . . . 304 | 274/300 (90%) | |
| | [WO200179449-A2, 25 OCT | | | |
| | 2001] | | | |
| AAG52267 | Arabidopsis thaliana protein | 71 . . . 320 | 101/261 (38%) | 4e−33 |
| | fragment SEQ ID NO: 66421 - | 99 . . . 340 | 133/261 (50%) | |
| | Arabidopsis thaliana, 348 aa. | | | |
| | [EP1033405-A2, 6 SEP 2000] | | | |
| AAG52266 | Arabidopsis thaliana protein | 71 . . . 320 | 101/261 (38%) | 4e−33 |
| | fragment SEQ ID NO: 66420 - | 125 . . . 366 | 133/261 (50%) | |
| | Arabidopsis thaliana, 374 aa. | | | |
| | [EP1033405-A2, 6 SEP 2000] | | | |
| AAG52265 | Arabidopsis thaliana protein | 71 . . . 320 | 101/261 (38%) | 4e−33 |
| | fragment SEQ ID NO: 66419 - | 218 . . . 459 | 133/261 (50%) | |
| | Arabidopsis thaliana, 467 aa. | | | |
| | [EP1033405-A2, 6 SEP 2000] | | | |

In a BLAST search of public sequence databases, the NOV101a protein was found to have homology to the proteins shown in the BLASTP data in Table 101D.

TABLE 101D
Public BLASTP Results for NOV101a

| | | NOV101a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9W0E2 | CG12091 PROTETN - Drosophila | 1 . . . 320 | 163/322 (50%) | 1e−83 |
| | melanogaster (Fruit fly), 321 aa. | 1 . . . 320 | 218/322 (67%) | |
| Q9W3R1 | CG15035 PROTEIN - Drosophila | 55 . . . 319 | 127/266 (47%) | 1e−64 |
| | melanogaster (Fruit fly), 374 aa. | 109 . . . 373 | 178/266 (66%) | |
| O18183 | W09D10.4 PROTEIN - | 4 . . . 320 | 136/331 (41%) | 2e−60 |
| | Caenorhabditis elegans, 330 aa. | 7 . . . 330 | 198/331 (59%) | |
| Q9VAH4 | CG7615 PROTEIN - Drosophila | 35 . . . 319 | 122/285 (42%) | 2e−56 |
| | melanogaster (Fruit fly), 314 aa. | 26 . . . 309 | 168/285 (58%) | |
| Q9SUK9 | HYPOTHETICAL 36.2 KDA | 71 . . . 320 | 101/261 (38%) | 1e−32 |
| | PROTEIN - Arabidopsis thaliana | 86 . . . 327 | 133/261 (50%) | |
| | (Mouse-ear cress), 335 aa. | | | |

PFam analysis predicts that the NOV101a protein contains the domains shown in the Table 110E.

TABLE 101E
Domain Analysis of NOV101a

| | | Identities/ | |
|---|---|---|---|
| | | Similarities | |
| | NOV101a Match | for the | Expect |
| Pfam Domain | Region | Matched Region | Value |
| PP2C: domain 1 of 1 | 147 . . . 191 | 13/48 (27%) | 0.26 |
| | | 36/48 (75%) | |

### Example 102

The NOV1102 clone was analyzed, and the nucleotide and predicted polypeptide sequences are shown in Table 102A.

TABLE 102A
NOV 102 Sequence Analysis

| | SEQ ID NO:289 | 523 bp | |
|---|---|---|---|
| NOV102a, | AGTCCCAGTACTATCAGCCATGGTCAACCACACCATGTTCTTCGACGTTGCTGTCGAC | | |
| CG59662-01 DNA Sequence | | | |
| | AGTGAGCCCTTGGACCACGTCTCCTTTGAGCTGTTTGCAGAAAAGTTTCCAAAGACAG | | |
| | CAGAAAACGTTCGTGCTCTGAGCACTGAAGAGAAAGGATTTGGTTATAAGGGTCCCTG | | |
| | CTTTCACAGAATTATACCAGCATTTATGTGTCAGGGTGGTGACTTCACGCACCATAAT | | |
| | GGCACTGGTGGCAAGTCCATCTACGGGGAGAAATTTGAAGATGAGAAATTTATCCTAA | | |
| | AGCGTACAGGTCCTGGCATCTTGTCCATGGCAAATTCTGGACCCAACACAAACTGTTC | | |
| | CGTTTTTTTCATCTGCACTGCCAAGACGGGGTGGTTGGATGGCAAGCATGTAGTCTTT | | |
| | GGCAAGGTGAAAGAAGGCATGAATATTTTGGAGGCCATAGAGCAATTTGGGTCCAGGA | | |
| | ATGGCAAGACCAGCAAGAAGACCACCATTGCTGACTGTGGACAGCTCTGGTAAGTTTGA | | |
| | ORF Start: ATG at 20 | | ORF Stop: TAA at 515 |
| | SEQ ID NO:290 | 165 aa | MW at 18237.7 kD |

| | |
|---|---|
| NOV102a, | MVNHTMFFDVAVDSEPLDHVSFELFAEKFPKTAENVRALSTEEKGFGYKGPCFHRIIP |
| CG59662-01 DNA Sequence | |
| | AFMCQGGDFTHHNGTGGKSIYGEKFEDEKFILKRTGPGILSMANSGPNTNCSVFFICT |
| | AKTGWLDGKHVVFGKVKEGMNILEAIEQFGSRNGKTSKKTTIADCGQLW |

Further analysis of the NOV102a protein yielded the following properties shown in Table 102B.

TABLE 102B
Protein Sequence Properties NOV102a

| | |
|---|---|
| PSort | 0.6400 probability located in microbody (peroxisome); |
| analysis: | 0.4500 probability located in cytoplasm; 0.1000 |
| | probability located in mitochondrial matrix space; |
| | 0.1000 probability located in lysosome (lumen) |
| SignalP | No Known Signal Sequence Predicted |
| analysis: | |

A search of the NOV102a protein against the Geneseq database, a proprietary database that contains sequences published in patents and patent publication, yielded several homologous proteins shown in Table 102C.

TABLE 102C
Geneseq Results for NOV102a

| | | NOV102a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU01195 | Human cyclophilin A protein - | 1 . . . 164 | 141/164 (85%) | 1e−80 |
| | Homo sapiens, 165 aa. | 1 . . . 164 | 148/164 (89%) | |
| | [WO200132876-A2, 10 MAY | | | |
| | 2001] | | | |
| AAW56028 | Calcineurin protein - Mammalia, | 1 . . . 164 | 141/164 (85%) | 1e−80 |
| | 165 aa. [WO9808956-A2, | 1 . . . 164 | 148/164 (89%) | |
| | 5 MAR 1998] | | | |
| AAG65275 | Haematopoietic stem cell | 2 . . . 164 | 140/163 (85%) | 5e−80 |
| | proliferation agent related human | 1 . . . 163 | 147/163 (89%) | |
| | protein #2 - Homo sapiens, 164 aa. | | | |
| | [JP2001163798-A, 19 JUN 2001] | | | |
| AAP90431 | Cyclophulin - Homo sapiens | 2 . . . 164 | 140/163 (85%) | 5e−80 |
| | (human), 164 aa. [EP326067-A, | 1 . . . 163 | 147/163 (89%) | |
| | 2 AUG 1989] | | | |
| AAG03831 | Human secreted protein, SEQ ID | 1 . . . 164 | 140/164 (85%) | 8e−80 |
| | NO: 7912 - Homo sapiens, 165 aa. | 1 . . . 164 | 147/164 (89%) | |
| | [EP1033401-A2, 6 SEP 2000] | | | |

In a BLAST search of public sequence databases, the NOV102a protein was found to have homology to the proteins shown in the BLASTP data in Table 102D.

TABLE 102D
Public BLASTP Results for NOV102a

| | | NOV102a | Identities/ | |
|---|---|---|---|---|
| Protein | | Residues/ | Similarities | |
| Accession | | Match | for the | Expect |
| Number | Protein/Organism/Length | Residues | Matched Portion | Value |
| CAC39529 | SEQUENCE 26 FROM PATENT | 1 . . . 164 | 141/164 (85%) | 4e−80 |
| | (Human), 165 aa. | 1 . . . 164 | 148/164 (89%) | |
| Q9BRU4 | PEPTIDYLPROLYL ISOMERASE | 1 . . . 164 | 140/164 (85%) | 2e−79 |
| | A (CYCLOPHILIN A) - Homo | 1 . . . 164 | 147/164 (89%) | |
| | sapiens (Human), 165 aa. | | | |
| P05092 | Peptidyl-prolyl cis-trans isomerase | 2 . . . 164 | 140/163 (85%) | 2e−79 |
| | A (EC 5.2.1.8) (PPIase) (Rotamase) | 1 . . . 163 | 147/163 (89%) | |
| | (Cyclophilin A) (Cyclosporin A- | | | |
| | binding protein) - Homo sapiens | | | |
| | (Human),, 164 aa. | | | |
| Q961X3 | PEPTIDYLPROLYL ISOMERASE | 1 . . . 164 | 140/164 (85%) | 5e−79 |
| | A (CYCLOPHILIN A) - Homo | 1 . . . 164 | 147/164 (89%) | |
| | sapiens (Human), 165 aa. | | | |
| P04374 | Peptidyl-prolyl cis-trans isomerase | 2 . . . 164 | 138/163 (84%) | 7e−79 |
| | A (EC 5.2.1.8) (PPIase) (Rotamase) | 1 . . . 163 | 147/163 (89%) | |
| | (Cyclophilin A) (Cyclosporin A- | | | |
| | binding protein) - Bos taurus | | | |
| | (Bovine), and, 163 aa. | | | |

PFam analysis predicts that the NOV102a protein contains the domains shown in the Table 102E.

TABLE 102E
Domain Analysis of NOV102a

| | | Identities/ | |
|---|---|---|---|
| | | Similarities | |
| | NOV102a Match | for the | Expect |
| Pfam Domain | Region | Matched Region | Value |
| pro_isomerase: | 5 . . . 165 | 105/180 (58%) | 4.2e−91 |
| domain 1 of 1 | | 141/180 (78%) | |

### Example 103

The NOV103 clone was analyzed, and the nucleotide and predicted polypeptide sequences are shown in Table 103A.

TABLE 103A
NOV103 Sequence Analysis

| | SEQ ID NO:291 | 8860 bp | |
|---|---|---|---|
| NOV103a, | GGATCCTTGAGGGCACTGGTGCGACTTTCAGGTGAGGTCTTAGCAGATGAAAGCGGCT | | |
| CG59773-01 DNA Sequence | | | |
| | GGCTGTGGCCCGCGCCAGTAGTGCTTTCTGCTCCGCACTCGCCGTGAGCCAGGTGTGC | | |
| | AACCGGATTTGGGGCGAGGGTCGCGCTGGCTACCTCGCATGCGCAGAGCCGGAAGCCC | | |
| | GCTGACCGGACTACAGCTCCCAGAAGAGCCTTGTGGAGGCCGCAGACGCGAAGCCGCT | | |
| | GGCGCCATCTTGAAATCTGATCCTCCATCCCCGAGGCTTTGCGTCTGCGCGGCCGGCC | | |
| | GCTGCTGCTCCGGGAGCCCAGTCTGCTAAAAGGGGAGGACGTTGAGGACGCGGCGGCT | | |
| | GGCGGGAGAGACAGCTGGGGAGAGACATGGCAGGGTCGGAGCGCGGCCTGCGCCTCTG | | |
| | TCACTCAGCATCCTCTTAGGCGTTTCCACGCCCGCCCCCTGCCCGAGGGGCGGGGCTG | | |
| | ACGGCTCTGGTACCCGGAGTCGGCGCGCGGGGCAGGGGCGCGCCCCTGCAGAGTGGGG | | |
| | ACCCCACTGGGCTGTGCCATGCTGACCGGAGACCACCGAGGCGGGAGACAGAGCGCGG | | |
| | CGAAGAGCCATTGAGTGGTCACCCAGTAGCCGCCGCCGCCGCCGCCTCGGGAAGCTTG | | |
| | CCACCCGCTAGGAGGGAAGATGAAGGAGATTTGCAGGATCTGTGCCCGAGAGCTGTGT | | |
| | GGAAACCAGCGGCGCTGGATCTTCCACACGGCGTCCAAGCTCAATCTCCAGGTTCTGC | | |
| | TTTCGCACGTCTTGGGCAAGGATGTCCCCCGCGATGGCAAAGCCGAGTTCGCTTGCAG | | |
| | CAAGTGTGCTTTCATGCTTGATCGAATCTATCGATTCGACACAGTTATTGCCCGGATT | | |
| | GAAGCGCTTTCTATTGAGCGCTTGCAAAAGCTGCTACTGGAGAAGGATCGCCTCAAGT | | |
| | TCTGCATTGCCAGTATGTATCGGAAGAATAACGATGACTCTGGCGCGGAGATCAAGGC | | |
| | GGGGAATGGGACGGTTGACATGTCCGTCTTACCCGATGCGAGATACTCTGCACTGCTC | | |
| | CAGGAGGACTTCGCCTATTCAGGGTTTGAGTGCTGGGTGGAGAATGAGGATCAGATCC | | |
| | AGGAGCCACACAGCTGCCATGGTTCAGAAGGCCCTGGAAACCGACCCAGGAGATGCCG | | |
| | TGGTTGTGCCGCTTTGCGGGTTGCTGATTCTGACTATGAAGCCATTTGTAAGGTACCT | | |
| | CGAAAGGTGGCCAGAAGTATCTCCTGCGGCCCTTCTAGCAGGTGGTCGACCAGCATTT | | |
| | GCACTGAAGAACCAGCGTTGTCTGAGGTTGGGCCACCCGACTTAGCAAGCACAAAGGT | | |
| | ACCCCCAGATGGAGAAAGCATGGAGGAAGAGACGCCTGGTTCCTCTGTGGAATCTTTG | | |
| | GATGCAAGCGTCCAGGCTAGCCCTCCACAACAGAAAGATGAGGAGACTGAGAGAAGTG | | |
| | CAAAGGAACTTGGAAAGTGTGACTGTTGTTCAGATGATCAGGCTCCGCAGCATGGGTG | | |
| | TAATCACAAGCTGGAATTAGCTCTTAGCATGATTAAAGGTCTTGATTATAAGCCCATC | | |
| | CAGAGCCCCCGAGGGAGCAGGCTTCCGATTCCAGTGAAATCCAGCCTACCTGGAGCCA | | |
| | AGCCTGGCCCTAGCATGACAGATGGAGTTAGTTCCGGTTTCCTTAACAGGTCTTTGAA | | |
| | ACCCCTTTACAAGACACCTGTGAGTTATCCCTTGGAGCTTTCAGACCTGCAGGAGCTG | | |
| | TGGGATGATCTCTGTGAAGATTATTTGCCGCTCCGGGTCCAGCCCATGACTGAAGAGT | | |
| | TGCTGAAACAACAAAAGCTGAATTCACATGAGACCACTATAACTCAGCAGTCTGTATC | | |
| | TGATTCCCACTTGGCAGAACTCCAGGAAAAAATCCAGCAAACAGAGGCCACCAACAAG | | |
| | ATTCTTCAAGAGAAACTTAATGAAATGAGCTATGAACTAAAGTGTGCTCAGGAGTCGT | | |
| | CTCAAAAGCAAGATGGTACAATTCAGAACCTCAAGGAAACTCTGAAAAGCAGGGAACG | | |
| | TGAGACTGAGGAGTTGTACCAGGTAATTGAAGGTCAAAATGACACAATGGCAAAGCTT | | |
| | CGAGAAATGCTGCACCAAAGCCAGCTTGGACAACTTCACAGCTCAGAGGGTACTTCTC | | |
| | CAGCTCAGCAACAGGTAGCTCTGCTTGATCTTCAGAGTGCTTTATTCTGCAGCCAACT | | |
| | TGAAATACAGAAGCTCCAGAGGGTGGTACGACAGAAAGAGCGCCAACTGGCTGATGCC | | |
| | AAACAATGTGTGCAATTTGTAGAGGCTGCAGCACACGAGAGTGAACAGCAGAAAGAGG | | |
| | CTTCTTGGAAACATAACCAGGAATTGCGAAAAGCCTTGCAGCAGCTACAAGAAGAATT | | |
| | GCAGAATAAGAGCCAACAGCTTCGTGCCTGGGAGGCTGAAAAATACAATGAGATTCGA | | |
| | ACCCAGGAACAAAACATCCAGCACCTAAACCATAGTCTGAGTCACAAGGAGCAGTTGC | | |
| | TTCAGGAATTTCGGGAGCTCCTACAGTATCGAGATAACTCAGACAAAACCCTTGAAGC | | |
| | AAATGAAATGTTGCTTGAGAAACTTCGCCAGCGAATACATGATAAAGCTGTTGCTCTG | | |
| | GAGCGGGCTATAGATGAAAAATTCTCTGCTCTAGAAGAGAAAGAAAAAGAACTGCGCC | | |
| | AGCTTCGTCTTGCTGTGAGAGAGCGAGATCATGACTTAGAGAGACTGCGCGATGTCCT | | |
| | CTCCTCCAATGAAGCTACTATGCAAAGTATGGAGAGTCTCCTGAGGGCCAAAGGCCTG | | |
| | GAAGTGGAACAGTTATCTACTACCTGTCAAAACCTCCAGTGGCTGAAAGAAGAAATGG | | |
| | AAACCAAATTTAGCCGTTGGCAGAAGGAACAAGAGAGTATCATTCAGCAGTTACAGAC | | |
| | GTCTCTTCATGATAGGAACAAAGAAGTGGAGGATCTTAGTGCAACACTGCTCTGCAAA | | |
| | CTTGGACCAGGGCAGAGTGAGATAGCAGAGGAGCTGTGCCAGCGTCTACAGCGAAAGG | | |
| | AAAGGATGCTGCAGGACCTTCTAAGTGATCGAAATAAACAAGTGCTGGAACATGAAAT | | |
| | GGAGATTCAAGGCCTGCTTCAGTCTGTGAGCACCAGGGAGCAGGAAAGCCAAGCTGCT | | |
| | GCAGAGAAGTTGGTGCAAGCCTTAATGGAAAGAAATTCAGAATTACAGGCCCTGCGCC | | |
| | AATATTTAGGAGGGAGAGACTCCCTGATGTCCCAAGCACCCATCTCTAACCAACAAGC | | |
| | TGAAGTTACCCCCACTGGCCGTCTTGGAAAACAGACTGATCAAGGTTCAATGCAGATA | | |
| | CCTTCCAGAGATGATAGCACTTCATTGACTGCCAAAGAGGATGTCAGCATACCCAGAT | | |
| | CCACATTAGGAGACTTGGACACAGTTGCAGGGCTGGAAAAAGAACTGAGTAATGCCAA | | |
| | AGAGGAACTTGAACTCATGGCTAAAAAAGAAAGAGAAAGTCAGATGGAACTTTCTGCT | | |
| | CTACAGTCCATGATGGCTGTGCAGGAAGAAGAGCTGCAGGTGCAGGCTGCTGATATGG | | |
| | AGTCTCTGACCAGGAACATACAGATTAAAGAAGATCTCATAAAGGACCTGCAAATGCA | | |
| | ACTGGTTGATCCTGAAGACATACCAGCTATGGAACGCCTGACCCAGGAAGTCTTACTT | | |
| | CTTCGGGAAAAAGTTGCTTCAGTAGAATCCCAGGGTCAAGAAATTTCAGGAAACCGAA | | |
| | GACAACAGTTGCTGCTGATGCTAGAAGGACTAGTAGATGAACGGAGTCGGCTCAATGA | | |
| | GGCCTTACAAGCAGAGAGACAGCTCTATAGCAGTCTGGTGAAGTTCCATGCCCATCCA | | |
| | GAGAGCTCTGAGAGAGACCGAACTCTGCAGGTGGAACTGGAAGGGGCTCAGGTGTTAC | | |
| | GCAGTCGGCTAGAAGAAGTTCTTGGAAGAAGCTTGGAGCGCTTAAACAGGCTGGAGAC | | |
| | CCTGGCCGCCATTGGAGGTGCAGCTGCAGGGGATGACACCGAAGATACAAGCACTGAG | | |
| | TTCACTGACAGTATTGAGGAGGAGGCTGCACACCATAGTCACCAGCAACTTGTCAAGG | | |
| | TGGCTTTGGAGAAAAGTCTGGCAACTGTGGAGACCCAGAACCCATCTTTTTCCCCTCC | | |
| | TTCTCCGATGGGAGGGGACAGTAACAGGTGTCTTCAGGAAGAAATGCTCCACCTGAGG | | |
| | GCTGAGTTCCACCAGCACTTAGAAGAGAAGAGGAAAGCTGAGGAGGAACTGAAGGAGC | | |
| | TAAAGGCTCAAATTGAGGAAGCAGGATTCTCCTCAGTGTCCCACATCAGGAACACCAT | | |
| | GCTGAGCCTTTGCCTTGAGAATGCGGAGCTGAAAGAGCAGATGGGAGAAGCAATGTCT | | |
| | GATGGATGGGAGATCGAGGAAGACAAGGAGAAGGGCGAGGTGATGGTTGAGACTGTGG | | |
| | TAACCAAAGAGGGTCTGAGTGAGAGTAGCCTTCAGGCTGAGTTCAGAAAGCTCCAGGG | | |
| | AAAACTGAAGAATGCCCACAATATCATCAACCTCCTCAAAGAACAACTTGTGCTGAGT | | |
| | AGCAAGGAAGGGAATAGTAAACTTACTCCAGAGCTCCTTGTGCATCTGACCAGCACCA | | |
| | TTGAAAGAATAAACACAGAACTGGTTGGTTCCCCTGGGAAGCACCAACACCAAGAGGA | | |
| | GGGGAATGTGACTGTGAGGCCTTTCCCCAGACCCCAGAGCCTTGACCTTGGGGCTACC | | |
| | TTCACAGTGGATGCCCACCAATTGGATAACCAGTCCCAGCCTCGTGACCCTGGGCCTC | | |
| | AGTCAGCGTTTAGCCTACCAGGGTCCACCCAGCACCTGCGCTCCCAGCTGTCACAATG | | |
| | CAAACAACGCTATCAAGATCTCCAGGAGAAGCTGCTGCTATCAGAAGCCACTGTCTTT | | |
| | GCTCAGGCTAACGAGCTGGAGAAATACAGAGTTATGCTTACAGGTGAATCCTTGGTGA | | |
| | AGCAGGACAGCCAGCAGATCCAGGTGGACCTCCAGGACCTGGGCTATGAGACTTGTGG | | |
| | CCGAAGCGAGAATGAGGCTGAACGGGAGGAAACCACCAGTCCTGAGTGTGAGGAGCAC | | |
| | AACAGCCTCAAGGAAATGGTCCTGATGGAGGGGCTGTGCTCTGAGCAGGGACGCCGGG | | |
| | GCTCAACACTGGCTAGTTCCTCTGAGAGGAAGCCCTTGGAGAACCAGCTAGGGAAGCA | | |
| | GGAAGAGTTCCGGGTATATGGAAAGTCAGAAAACATCTTGGTCCTACGAAAGGACATC | | |
| | AAAGATCTGAAGGCCCAGCTGCAGAATGCCAACAAGGTCATTCAAAACCTCAAGAGCC | | |
| | GGGTCCGGTCCCTCTCAGTTACAAGTGATTATTCGTCTAGTCTGGAAAGACCCCGGAA | | |
| | GCTGAGAGCTGTTGGCACCTTGGAGGGGTCTTCACCTCATAGTGTCCCTGATGAGGAT | | |
| | GAGGGGTGGCTGTCTGATGGCACTGGGGCTTTCTACTCTCCAGGGCTTCAGGCCAAAA | | |
| | AGGACCTGGAGAGTCTCATCCAGAGAGTATCCCAGCTGGAGGCCCAGCTCCCAAAAAA | | |
| | TGGACTAGAAGAGAAGCTGGCTGAGGAGCTGAGATCAGCCTCGTGGCCTGGGAAATAT | | |
| | GATTCCCTGATTCAGGATCAGGCCCGGGAACTGTCTTACCTACGGCAAAAAATACGAG | | |
| | TGAGGATCTCCTAAGGAGCAATGACATTGACTACTACCTGGGACAGAGCTTCCGGGAG | | |
| | TGAGGATCTCCTAAGGAGCAATGACATTGACTACTACCTGGGACAGAGCTTCCGGGAG | | |
| | CAACTCGCCCAGGGAAGCCAGCTGACAGAGAGGCTCACCAGCAAACTCAGCACCAAGG | | |
| | ATCATAAAAGTGAGAAAGATCAAGCTGGACTTGAGCCACTGGCCCTCAGGCTCAGCAG | | |
| | GGAGCTGCAGGAGAAGGAGAAAGTGATTGAAGTCCTGCAGGCCAAGCTGGATGCTCGG | | |
| | TCCCTCACACCCTCCAGCAGCCATGCCTTGTCTGACTCCCACCGCTCTCCCAGCAGCA | | |
| | CCTCTTTCCTGTCTGATGAACTGGAAGCCTGCTCTGACATGGACATAGTCAGCGAGTA | | |
| | CACACACTATGAAGAGAAGAAAGCTTCTCCCAGTCACTCAGATTCCATCCATCATTCG | | |
| | AGTCATTCTGCTGTGTTGTCTTCTAAACCATCATCAACCAGTGCATCTCAGGGGGCTA | | |
| | AGGCCGAATCCAACAGCAACCCCATCAGCTTGCCAACTCCCCAGAATACCCCCAAGGA | | |
| | GGCCAACCAGGCCCATTCAGGCTTTCATTTTCACTCCATACCCAAGCTGGCTAGCCTT | | |
| | CCTCAGGCACCATTGCCCTCAGCTCCATCCAGCTTCCTGCCTTTCAGCCCCACTGGCC | | |
| | CTCTCCTCCTTGGCTGCTGTGAGACACCAGTGGTCTCCTTGGCTGAGGCTCAGCAGGA | | |
| | GCTACAGATGCTGCAGAAGCAGTTGGGAGAAAGTGCCAGCACTGTTCCTCCTGCTTCC | | |
| | ACAGCTACATTGCTGAGCAACGACTTGGAAGCCGACTCTTCCTACTACCTCAACTCTG | | |
| | CCCAGCCTCACTCTCCTCCAAGGGGCACCATAGAACTGGGAAGAATCCTAGAGCCTGG | | |
| | GTACCTGGGCAGCAGTGGCAAGTGGGATGTGATGAGGCCTCAGAAAGGGAGTGTATCT | | |
| | GGGGACCTATCCTCAGGCTCCTCTGTGTACCAGCTTAACTCCAAACCCACAGGGGCTG | | |
| | ACCTGCTGGAAGAGCATCTTGGTGAAATCCGGAACCTGCGCCAGCGCCTGGAGGAGTC | | |
| | CATCTGCATCAATGACCGCCTACGGGAGCAACTGGAACACCGGCTGACCTCTACTGCT | | |
| | CGTGGAAGGGGATCCACTTCTAACTTCTACAGTCAGGGCCTGGAGTCCATACCTCAGC | | |
| | TCTGCAATGAGAACAGAGTCCTCAGGGAAGACAATCGAAGACTTCAGGCTCAACTGAG | | |
| | TCATGTTTCCAGAGAGCACTCCCAGGAAACAGAAAGCCTGAGGGAGGCTCTGCTGTCC | | |
| | TCTCGATCCCACCTTCAAGAGCTGGAAAAGGAGCTGGAGCACCAGAAGGTGGAAAGGC | | |
| | AGCAGCTTTTGGAAGACTTGAGGGAGAAGCAGCAAGAGGTCTTGCATTTCAGGGAGGA | | |
| | ACGTCTTTCCCTCCAGGAAAACGACTCCAGACTGCAGCACAAGCTGGTTCTCCTGCAG | | |
| | CAACAGTGTGAAGAGAAACAGCAGCTCTTTGAGTCCCTCCAGTCAGAGCTACAAATCT | | |
| | ACGAGGCACTTTATGGCAATTCCAAGAAGGGGCTGAAAGGCTTGGGTTTGGATACTTC | | |
| | TCCAGTAATGAAGACCCCTCCCAAGCTAGAGGGTGATGCTACTGATGGCTCCTTTGCC | | |
| | AATAAGCATGGCCGCCATGTCATTGGCCACATTGATGACTACAGTGCCCTAAGACAGC | | |
| | AGATTGCGGAGGGCAAGCTGCTGGTCAAAAAGATAGTGTCTCTTGTGAGATCAGCGTG | | |
| | CAGCTTCCCTGGCCTTGAAGCCCAAGGCACAGAGGTGCTAGGCAGCAAAGGTATTCAT | | |
| | GAGCTTCGGAGCAGCACCAGTGCCCTGCACCATGCCCTAGAGGAGTCGGCTTCCCTCC | | |
| | TCACCATGTTCTGGAGAGCAGCCCTGCCAAGCACCCACATCCCTGTGCTGCCTGGCAA | | |
| | AGTGGGAGAATCAACAGAAAGGGAACTTCTGGAACTGAGAACCAAAGTATCCAAACAG | | |
| | GAGCGGCTCCTTCAGAGCACAACTGAGCATCTGAAGAACGCCAACCAGCAGAAGGAGA | | |
| | GCATGGAGCAGTTCATCGTCAGCCAGCTAACCAGAACACATGATGTTTTAAAGAAGGC | | |
| | AAGGACTAACTTAGAGGTGAAATCCCTAAGGGCTCTGCCATGTACTCCAGCCTTGTGA | | |
| | CCCTTGCCTTCCAGGAACCATGCAAGAAGCGCAGCCACCAGAAGTCCTTAAAACAGCA | | |
| | GGAAAGGTGGGCCTGTCCCCCTTTTGTGCAGCTACCTATCTGCTGAGGAGCATCTGGG | | |
| | CCTCATTCCTCCAAGTCCACGGGAGGGTCCAGAAGAGGGAGTCAGAGATGTATCCTGG | | |
| | TGGAGCTGGGAGAAAGGCAGAAAGCCTTTCTGACAGCTATGGAATACGATTAGCCAAG | | |
| | GTCCACTTGGCCCAGCACTAAGAAAAAGATGCGTAGTTTGCACAGAAGGTTTTGTGAT | | |
| | CCTGCCTCTCAACAGCCCCAGCAGCTTGGGAACTAGCAAGAGCACATTTCTTGCCTCA | | |
| | TCAGCTGTCCTGAGATGGAAAACTCAGTGGATATAGGACCCTGATTCCGATGAAAGGG | | |
| | GCACGTGGTCCCAATGCTGGAGCTCCTCTGGCAGGTTCTAAAAGCACACTACTGAGCA | | |
| | GCGGTGCCCTGCCGGACACTGCTGGCGGGGGCTCAGTGAGCACTACTCACAGATCCAC | | |
| | ACCTGACCCTGTTGGGTCGAGTCAGGCTGGGCCTTGGTCTGCACTGTAGCACCTGTGT | | |
| | TCTTTGAGTTCACATCATGAATGTGGTGACTTCCCAGATACCATCTCAGGCTTAACCT | | |
| | AGCACATCCTATTTCTTTTCTTCTATGATATCCAAATTGGACTGACCTCACTTCAAAG | | |
| | TTGCTGTCCCATTTTGTCACCCTATCTTATCTCGGGGAAATTGCAGACTGATGGCCAG | | |
| | ACCAACTCTGTTGAAATTCTTGCATAGAGCAAACCTGTGCTCATTTTTAAGTGGCATG | | |
| | GGAGAGGCCCCCAGCCTAGTAAAGCCTAGTCTGTGTCTTCACAGTGCTGGTAGAATGT | | |
| | GTTTGTGTGTATAAATATATGATATAGATTTATATATGTTGCTAACGCCATATATTGA | | |
| | AGGCCAACATAACTGGTGGACAGGGTGGGTGACAGAAAATGAAAGCCTTTTTGGTGAT | | |
| | TGTTAAAGCAAGATGTGTATAAAGAAATAAATAGTTTTTCTTTC | | |
| | ORF Start: ATG at 658 | | ORF Stop: TGA at 7828 |
| | SEQ ID NO:292 | 2390 aa | MW at 268843.7 kD |
| NOV103a, | MKEICRICARELCGNQRRWIFHTASKLNLQVLLSHVLGKDVPRDGKAEFACSKCAFML | | |
| CG59773-01 Protein | | | |
| Sequence | DRIYRFDTVIARIEALSIERLQKLLLEKDRLKFCIASMYRKNNDDSGAEIKAGNGTVD | | |
| | MSVLPDARYSALLQEDFAYSGFECWVENEDQIQEPHSCHGSEGPGNRPRRCRGCAALR | | |
| | VADSDYEAICKVPRKVARSISCGPSSRWSTSICTEEPALSEVGPPDLASTKVPPDGES | | |
| | MEEETPGSSVESLDASVQASPPQQKDEETERSAKELGKCDCCSDDQAPQHGCNHKLEL | | |
| | ALSMIKGLDYKPIQSPRGSRLPIPVKSSLPGAKPGPSMTDGVSSGFLNRSLKPLYKTP | | |
| | VSYPLELSDLQELWDDLCEDYLPLRVQPMTEELLKQQKLNSHETTITQQSVSDSHLAE | | |
| | LQEKIQQTEATNKILQEKLNEMSYELKCAQESSQKQDGTIQNLKETLKSRERETEELY | | |
| | QVIEGQNDTMAKLREMLHQSQLGQLHSSEGTSPAQQQVALLDLQSALFCSQLEIQKLQ | | |
| | RVVRQKERQLADAKQCVQFVEAAAHESEQQKEASWKHNQELRKALQQLQEELQNKSQQ | | |
| | LRAWEAEKYNEIRTQEQNIQHLNHSLSHKEQLLQEFRELLQYRDNSDKTLEANEMLLE | | |
| | KLRQRIHDKAVALERAIDEKFSALEEKEKELRQLRLAVRERDHDLERLRDVLSSNEAT | | |
| | MQSMESLLRAKGLEVEQLSTTCQNLQWLKEEMETKFSRWQKEQESIIQQLQTSLHDRN | | |
| | KEVEDLSATLLCKLGPGQSEIAEELCQRLQRKERMLQDLLSDRNKQVLEHEMEIQGLL | | |
| | QSVSTREQESQAAAEKLVQALMERNSELQALRQYLGGRDSLMSQAPISNQQAEVTPTG | | |
| | RLGKQTDQGSMQIPSRDDSTSLTAKEDVSIPRSTLGDLDTVAGLEKELSNAKEELELM | | |
| | AKKERESQMELSALQSMMAVQEEELQVQAADMESLTRNIQIKEDLIKDLQMQLVDPED | | |
| | IPAMERLTQEVLLLREKVASVESQGQEISGNRRQQLLLMLEGLVDERSRLNEALQAER | | |
| | QLYSSLVKFHAHPESSERDRTLQVELEGAQVLRSRLEEVLGRSLERLNRLETLAAIGG | | |
| | AAAGDDTEDTSTEFTDSIEEEAAHHSHQQLVKVALEKSLATVETQNPSFSPPSPMGGD | | |
| | SNRCLQEEMLHLRAEFHQHLEEKRKAEEELKELKAQIEEAGFSSVSHIRNTMLSLCLE | | |
| | NAELKEQMGEAMSDGWEIEEDKEKGEVMVETVVTKEGLSESSLQAEFRKLQGKLKNAH | | |
| | NIINLLKEQLVLSSKEGNSKLTPELLVHLTSTIERINTELVGSPGKHQHQEEGNVTVR | | |
| | PFPRPQSLDLGATFTVDAHQLDNQSQPRDPGPQSAFSLPGSTQHLRSQLSQCKQRYQD | | |
| | LQEKLLLSEATVFAQANELEKYRVMLTGESLVKQDSKQIQVDLQDLGYETCGRSENEA | | |
| | EREETTSPECEEHNSLKEMVLMEGLCSEQGRRGSTLASSSERKPLENQLGKQEEFRVY | | |
| | GKSENILVLRKDIKDLKAQLQNANKVIQNLKSRVRSLSVTSDYSSSLERPRKLRAVGT | | |
| | LEGSSPHSVPDEDEGWLSDGTGAFYSPGLQADDKLESLIQRVSQLEAQLPKNGLEEKL | | |
| | AEELRSASWPGKYDSLIQDQARELSYLRQKIREGRGICYLITRHAKDTVKSFEDLLRS | | |
| | NDIDYYLGQSFREQLAQGSQLTERLTSKLSTKDHKSEKDQAGLEPLALRLSRELQEKE | | |
| | KVIEVLQAKLDARSLTPSSSHALSDSHRSPSSTSFLSDELEACSDMDIVSEYTHYEEK | | |
| | KASPSHSDSIHHSSHSAVLSSKPSSTSASQGAKAESNSNPISLPTPQNTPKEANQAHS | | |
| | GFHFHSIPKLASLPQAPLPSAPSSFLPFSPTGPLLLGCCETPVVSLAEAQQELQMLQK | | |
| | QLGESASTVPPASTATLLSNDLEADSSYYLNSAQPHSPPRGTIELGRILEPGYLGSSG | | |
| | KWDVMRPQKGSVSGDLSSGSSVYQLNSKPTGADLLEEHLGEIRNLRQRLEESICINDR | | |
| | LREQLEHRLTSTARGRGSTSNFYSQGLESIPQLCNENRVLREDNRRLQAQLSHVSREH | | |
| | SQETESLREALLSSRSHLQELEKELEHQKVERQQLLEDLREKQQEVLHFREERLSLQE | | |
| | NDSRLQHKLVLLQQQCEEKQQLFESLQSELQIYEALYGNSKKGLKGLGLDTSPVMKTP | | |
| | PKLEGDATDGSFANKHGRHVIGHIDDYSALRQQIAEGKLLVKKIVSLVRSACSFPGLE | | |
| | AQGTEVLGSKGIHELRSSTSALHHALEESASLLTMFWRAALPSTHIPVLPGKVGESTE | | |
| | RELLELRTKVSKQERLLQSTTEHLKNANQQKESMEQFIVSQLTRTHDVLKKARTNLEV | | |
| | KSLRALPCTPAL | | |
| | SEQ ID NO:293 | 7161 bp | |
| NOV103b, | GTTGAGGGGGCAATCGGGCACGCTCCTCCCCATGGGTTGCCCATCATGTCTAATGGAT | | |
| CG59773-02 DNA Sequence | | | |
| | ATCGCACTCTGTCCCAGCACCTCAATGACCTGAAGAAGGAGAACTTCAGCCTCAAGCT | | |
| | GCGCATCTACTTCCTGGAGGAGCGCATGCAACAGAAGTATGAGGCCAGCCGGGAGGAC | | |
| | ATCTACAAGCGGAACATTGAGCTGAAGGTTGAAGTGGAGAGCTTGAAACGAGAACTCC | | |
| | AGGACAAGAAACAGCATCTGGATAAAACATGGGCTGATGTGGAGAATCTCAACAGTCA | | |
| | GAATGAAGCTGAGCTCCGACGCCAGTTTGAGGAGCGACAGCAGGAGACGGAGCATGTT | | |
| | TATGAGCTCTTGGAGAATAAGATCCAGCTTCTGCAGGAGGAATCCAGGCTAGCAAAGA | | |
| | ATGAAGCTGCGCGGATGGCAGCTCTGGTGGAAGCAGAGAAGGAGTGTAACCTGGAGCT | | |
| | CTCAGAGAAACTGAAGGGAGTCACCAAAAACTGGGAAGATGTACCAGGAGACCAGGTC | | |
| | AAGCCCGACCAATACACTGAGGCCCTGGCCCAGAGGGACAGGAGAATTGAAGAACTGA | | |
| | ATCAGAGCCTGGCTGCCCAGGAGAGGCTTGTAGAACAGCTATCTCGGGAGAAACAACA | | |
| | ACTGCTACATCTGTTGGAGGAGCCAACTAGCATGGAAGTGCAGCCCATGACTGAAGAG | | |
| | TTGCTGAAACAACAAAAGCTGAATTCACATGAGACCACTATAACTCAGCAGTCTGTAT | | |
| | CTGATTCCCACTTGGCAGAACTCCAGGAAAAAATCCAGCAAACAGAGGCCACCAACAA | | |
| | GATTCTTCAAGAGAAACTTAATGAAATGAGCTATGAACTAAAGTGTGCTCAGGAGTCG | | |
| | TCTCAAAAGCAAGATGGTACAATTCAGAACCTCAAGGAAACTCTGAAAAGCAGGGAAC | | |
| | GTGAGACTGAGGAGTTGTACCAGGTAATTGAAGGTCAAAATGACACAATGGCAAAGCT | | |
| | TCGAGAAATGCTGCACCAAAGCCAGCTTGGACAACTTCAGAGCTCAGAGGGTACTTCT | | |
| | CCAGCTCAGCAACAGGTAGCTCTGCTTGATCTTCAGAGTGCTTTATTCTGCAGCCAAC | | |
| | TTGAAATACAGAAGCTCCAGAGGGTGGTACGACAGAAAGAGCGCCAACTGGCTGATGC | | |
| | CAAACAATGTGTGCAATTTGTAGAGGCTGCAGCACACGAGAGTGAACAGCAGAAAGAG | | |
| | GCTTCTTGGAAACATAACCAGGAATTGCGAAAAGCCTTGCAGCAGCTACAAGAAGAAT | | |
| | TGCAGAATAAGAGCCAACAGCTTCGTGCCTGGGAGGCTGAAAAATACAATGAGATTCG | | |
| | AACCCAGGAACAAAACATCCAGCACCTAAACCATAGTCTGAGTCACAAGGAGCAGTTG | | |
| | CTTCAGGAATTTCGGGAGCTCCTACAGTATCGAGATAACTCAGACAAAACCCTTGAAG | | |
| | CAAATGAAATGTTGCTTGAGAAACTTCGCCAGCGAATACATGATAAAGCTGTTGCTCT | | |
| | GGAGCGGGCTATAGATGAAAAATTCTCTGCTCTAGAAGAGAAAGAAAAAGAACTGCGC | | |
| | CAGCTTCGTCTTGCTGTGAGAGAGCGAGATCATGACTTAGAGAGACTGCGCGATGTCC | | |
| | TCTCCTCCAATGAAGCTACTATGCAAAGTATGGAGAGTCTCCTGAGGGCCAAAGGCCT | | |
| | GGAAGTGGAACAGTTATCTACTACCTGTCAAAACCTCCAGTGGCTGAAAGAAGAAATG | | |
| | GAAACCAAATTTAGCCGTTGGCAGAAGGAACAAGAGAGTATCATTCAGCAGTTACAGA | | |
| | CGTCTCTTCATGATAGGAACAAAGAAGTGGAGGATCTTAGTGCAACACTGCTCTGCAA | | |
| | ACTTGGACCAGGGCAGAGTGAGATAGCAGAGGAGCTGTGCCAGCGTCTACAGCGAAAG | | |
| | GAAAGGATGCTGCAGGACCTTCTAAGTGATCGAAATAAACAAGTGCTGGAACATGAAA | | |
| | TGGAGATTCAAGGCCTGCCTCAGTCTGTGAGCACCAGGGAGCAGGAAAGCCAAGCTGC | | |
| | TGCAGAGAAGTTGGTGCAAGCCTTAATGGAAAGAAATTCAGAATTACAGGCCCTGCGC | | |
| | CAATATTTAGGAGGGAGAGACTCCCTGATGTCCCAAGCACCCATCTCTAACCAACAAG | | |
| | CTGAAGTTACCCCCACTGGCCGTCTTGGAAAACAGACTGATCAAGGTTCAATGCAGAT | | |
| | ACCTTCCAGAGATGATAGCACTTCATTGACTGCCAAAGAGGATGTCAGCATACCCAGA | | |
| | TCCACATTAGGAGATTTGGACACAGTTGCAGGGCTGGAAAAAGAACTGAGTAATGCCA | | |
| | AAGAGGAACTTGAACTCATGGCTAAAAAAGAAAGAGAATCACAGATGGAACTTTCTGC | | |
| | TCTACAGTCCATGATGGCTGTGCAGGAAGAAGAGCTGCAGGTGCAGGCTGCTGATATG | | |
| | GAGTCTCTGACCAGGAACATACAGATTAAAGAAGATCTCATAAAGGACCTGCAAATGC | | |
| | AACTGGTTGATCCTGAAGACATACCAGCTATGGAACGCCTGACCCAGGAAGTCTTACT | | |
| | TCTTCGGGAAAAAGTTGCTTCAGTAGAATCCCAGGGTCAAGAAATTTCAGGAAACCGA | | |
| | AGACAACAGCAGTTGCTGCTGATGCTAGAAGGACTAGTAGATGAACGGAGTCGGCTCA | | |
| | ATGAGGCCTTACAAGCAGAGAGACAGCTCTATAGCAGTCTGGTGAAGTTCCATGCCCA | | |
| | TCCAGAGAGCTCTGAGAGAGACCGAACTCTGCAGGTGGAACTGGAAGGGGCTCAGGTG | | |
| | TTACGCAGTCGGCTAGAAGAAGTTCTTGGAAGAAGCTTGGAGCGCTTAAACAGGCTGG | | |
| | AGACCCTGGCCGCCATTGGAGGTGCAGCTGCAGGGGATGACACCGAAGATACAAGCAC | | |
| | TGAGTTCACTGACAGTATTGAGGAGGAGGCTGCACACCATAGTCACCAGCAACTTGTC | | |
| | AAGGTGGCTTTGGAGAAAAGTCTGGCAACTGTGGAGACCCAGAACCCATCTTTTTCCC | | |
| | CTCCTTCTCCGATGGGAGGGGACAGTAACAGGTGTCTTCAGGAAGAAATGCTCCACCT | | |
| | GAGGGCTGAGATCCACCAGCACTTAGAAGAGAAGAGGAAAGCTGAGGAGGAACTGAAG | | |
| | GAGCTAAAGGCTCAAATTGAGGAAGCAGGATTCTCCTCAGTGTCCCACATCAGGAACA | | |
| | CCATGCTGAGCCTTTGCCTTGAGAATGCGGAGCTGAAAGAGCAGATGGGAGAAACAAT | | |
| | GTCTGATGGATGGGAGATCGAGGAAGACAAGGAGAAGGGCGAGGTGATGGTTGAGACT | | |
| | GTGGTAACCAAAGAGGGTCTGAGTGAGAGTAGCCTTCAGGCTGAGTTCAGAAAGCTCC | | |
| | AGGGAAAACTGAAGAATGCCCACAATATCATCAACCTCCTCAAAGAACAACTTGTGCT | | |
| | GAGTAGCAAGGAAGGGAATAGTAAACTTACTCCAGAGCTCCTTGTGCATCTGACCAGC | | |
| | ACCATCGAAAGAATAAACACAGAACTGGTTGGTTCCCCTGGGAAGCACCAACACCAAG | | |
| | AGGAGGGGAATGTGACTGTGAGGCCTTTCCCCAGACCCCAGAGCCTTGACCTTGGGGC | | |
| | TACCTTCACAGTGGATGCCCACCAACAGTTGGATAACCAGTCCCAGCCTCGTGACCCT | | |
| | GGGCCTCAGCCAGCGTTTAGCCTACCAGGGTCCACCCAGCACCTGCGCTCCCAGCTGT | | |
| | CACAATGCAAACAACGCTATCAAGATCTCCAGGAGAAGCTGCTGCTATCAGAAGCCAC | | |
| | TGTCTTTGCTCAGGCTAACGAGCTGGAGAAATACAGAGTTATGCTTAGTGAATCCTTG | | |
| | GTGAAGCAGGACAGCAAGCAGATCCAGGTGGACTTCCAGGACCTGGGCTATGAGACTT | | |
| | GTGGCCGAAGCGAGAATGAGGCTGAACGGGAGGAAACCACCAGTCCTGAGTGTGAGGA | | |
| | GCACAACAGCCTCAAGGAAATGGTCCTGATGGAGGGGCTGTGCTCTGAGCAGGGACGC | | |
| | CGGGGCTCAACACTGGCTAGTTCCTCTGAGAGGAAGCCCTTGGAGAACCAGCTAGGGA | | |
| | AGCAGGAAGAGTTCCGGGTATATGGAAAGTCAGAAAACATCTTGGTCCTACGAAAGGA | | |
| | CATCGAAGATCTGAAGGCCCAGCTGCAGAATGCCAACAAGGTCATTCAAAACCTCAAG | | |
| | AGCCGGGTCCGGTCCCTCTCAGTTACAAGTGATTATTCGTCTAGTCTGGAAAGACCCC | | |
| | GGAAGCTGAGAGCTGTTGGCACCTTGGAGGGGTCTTCACCTCATAGTGTCCCTGATGA | | |
| | GGATGAGGGGTGGCTGTCTGATGGCACTGGGGCTTTCTACTCTCCAGGGCTTCAGGCC | | |
| | AAAAAGGACCTGGAGAGTCTCATCCAGAGAGTATCCCAGCTGGAGGCCCAGCTCCCAG | | |
| | AAAATGGACTAGAAGAGAAGCTGGCTGAGGAGCTGAGATCAGCCTCGTGGCCTGGGAA | | |
| | ATATGATTCCCTGATTCAGGATCAGGCCCGGGAACTGTCTTACCTACGGCAAAAAATA | | |
| | CGAGAAGGGAGAGGTATTTGTTATCTTATCACCCAGCATGCAAAAGATACAGTAAAAT | | |
| | CTTTTGAGGATCTCCTAAGGAGCAATGACATTGACTACTACCTGGGACAGAGCTTCCG | | |
| | GGAGCAACTCGCCCAGGGAAGCCAGCTGACAGAGAGGCTCACCAGCAAACTCAGCACA | | |
| | GAGGATCATAAAAGTGAGAAAGATCAAGCTGGACTTGAGCCACTGGCCCTCAGGCTCA | | |
| | GCAGGGAGCTGCAGGAGAAGGAGAAAGTGATTGAAGTCCTGCAGGCCAAGCTGGATGC | | |
| | TCGGTCCCTCACACCCTCCAGCAGCCGTGCCTTGTCTGACTCCCACCGCTCTCCCAGC | | |
| | AGCACCTCTTTCCTGTCTGATGATCTGGAAGCCTGCTCTGACATGGACATAGTCAGCG | | |
| | AGTACACACACTATGAAGAGAAGAAAGCTTCTCCCAGTCACTCAGGTAGCAGTGCATC | | |
| | TCAGGGGGCTAAGGCCGAATCCAACAGCAACCCCATCAGCTTGCCAACTCCCCAGAAT | | |
| | ACCCCCAAGGAGGCCAACCAAGCCCATTCAGGCTTTCATTTTCACTCCATACCCAAGC | | |
| | TGGCTAGCCTTCCTCAGGCACCATTGCCCTCAGCTCCATCCAGCTTCCTGCCTTTCAG | | |
| | CCCCACTGGCCCTCCCCTCCTTGGCTGCTGTGAGACACCAGAGGTCTCCTTGGCTGAG | | |
| | TCTCAGCAGGAGCTACAGATGCTGCAGAAGCAGTTGGGAGAAAGTAGCACTGTTCCTC | | |
| | CTGCTTCCACAGCTACATTGCTGAGCAACGACTTGGAAGCCGACTCTTCCTACTACCT | | |
| | CAACTCTGCCCAGCCTCACTCTCCTCCAAGGGGCACCATAGAACTGGGAAGAATCCTA | | |
| | GAGCCTGGGTACCTGGGCAGCAGTGGCAAGTGGGATGTGATGAGGCCTCAGAAAGGGA | | |
| | GTGTATCTGGGGACCTATCCTCAGGCTCCTCTGTGTACCAGCTTAACTCCAAACCCAC | | |
| | AGGGGCTGACCTGCTGGAAGAGCATCTTGGTGAAATCTGGAACCTGCGCCAGCGCCTG | | |
| | GAGGAGTCCATCTGCATCAATGACTGCCTACGGGAGCAACTGGAACACCGGCTGACCT | | |
| | CTACTGCTCGTGGAAGGGGATCCACTTCTAACTTCTACAGTCAGGGCCTGGAGTCCAT | | |
| | ACCTCAGCTCTGCAATGAGAACAGAGTCCTCAGGGAAGAAAATCGAAGACTTCAGGCT | | |
| | CAACTGAGTCATGTTTCCAGAGGTCACTCCCAGGAAACAGAAAGCCTGAGGGAGGCTC | | |
| | TGCTGTCCTCTCGATCCCACCTTCAAGAGCTGGAAAAGGAGCTGGAGCACCAGAAGGT | | |
| | GGAAAGGCAGCAGCTTTTGGAAGACTTGAGGGAGAAGCAGCAAGAGGTCTTGCATTTC | | |
| | AGGGAGGAACGTCTTTCCCTCCAGGAAAACGACTCCAGACTGCAGCACAAGCTGGTTC | | |
| | TCCTGCAGCAACAGTGTGAAGAGAAACAGCAGCTCTTTGAGTCCCTCCAGTCAGAGCT | | |
| | ACAAATCTACGAGGCACTTTATGGCAATTCCAAGAAGGGGCTGAAAGCTTACAGCCTG | | |
| | GATGCCTGTCACCAAATCCCTTTGAGCAGTGACCTGAGCCACCTGGTGGCAGAGGTAC | | |
| | AAGCTCTGAGAGGGCAGCTGGAGCAGAGCATTCAGGGGAACAATTGTCTGCGACTGCA | | |
| | GCTGCAACAGCAGCTGGAGAGCGGTGCTGGCAAAGCCAGCCTCAGCCCCTCCTCCATT | | |
| | AACCAGAACTTCCCAGCCAGCACTGACCCTGGAAACAAGCAGCTGCTCCTCCAAGGTT | | |
| | CAGCTGTGTCCCCTCCAGTCCGGGATGTTGGTATGAATTCCCCAGCTCTGGTCTTCCC | | |
| | CAGCTCTGCTTCCTCTACTCCTGGCTCAGATTCAGTTGTGTTGTCATTTTCTTTTTCA | | |
| | GGCTTGGGTTTGGATACTTCTCCAGTAATGAAGACCCCTCCCAAGCTAGAGGGTGATG | | |
| | CTACTGATGGCTCCTTTGCCAATAAGCATGGCCGCCATGTCATTGGCCACATTGATGA | | |
| | CTACAGTGCCCTAAGACAGCAGATTGCGGAGGGCAAGCTGCTGGTCAAAAAGATAGTG | | |
| | TCTCTTGTGAGATCAGCGTGCAGCTTCCCTGGCCTTGAAGCCCAAGGCACAGAGGGCA | | |
| | GCAAAGGCATTCATGAGCTTCGGAGCAGCACCAGTGCCCTGCACCATGCCCTAGAGGA | | |
| | GTCGGCTTCCCTCCTCACCATGTTCTGGAGAGCGGCCCTGCCAAGCACCCACATCCCT | | |
| | GTGCTGCCTGGCAAACAGGGAGAATCAACAGAAAGGGAACTTCTGGAACTGAGAACCA | | |
| | AAGTATCCAAACAGGAGCAGCTCCTTCAGAGCACAACTGAGCATCTGAAGAACGCCAA | | |
| | CCAGCAGAAGGAGAGCATGGAACAGTTCATTGTCAGCGTAACCAGAACACATGATGTT | | |
| | TTAAAGAAGGCAAGGACTAACTTAGAGGTGAAATCCCTAAGGGCTCTGCCGTGTACTC | | |
| | CAGCCTTGTGACCCTTGCCTTCCAGGAACCATGCAAGAAGCGCAGCCACCAGAAGTCC | | |
| | TTAAAACAGCAGGAAGGTGAGCCTGTCCCCCTTTTGTGCAGCTACCTATCTGCTGAG | | |
| | GAGCATCTGGGCCTCATTCCTCCAAGT | | |
| | ORF Start: ATG at 46 | | ORF Stop: TGA at 7027 |
| | SEQ ID NO:294 | 2327 aa | MW at 263034.6 kD |
| NOV103b, | MSNGYRTLSQHLNDLKKENFSLKLRIYFLEERMQQKYEASREDIYKRNIELKVEVESL | | |
| CG59773-02 Protein | | | |
| Sequence | KRELQDKKQHLDKTWADVENLNSQNEAELRRQFEERQQETEHVYELLENKIQLLQEES | | |
| | RLAKNEAARMAALVEAEKECNLELSEKLKGVTKNWEDVPGDQVKPDQYTEALAQRDRR | | |
| | IEELNQSLAAQERLVEQLSREKQQLLHLLEEPTSMEVQPMTEELLKQQKLNSHETTIT | | |
| | QQSVSDSHLAELQEKIQQTEATNKILQEKLNEMSYELKCAQESSQKQDGTIQNLKETL | | |
| | KSRERETEELYQVIEGQNDTMAKLREMLHQSQLGQLQSSEGTSPAQQQVALLDLQSAL | | |
| | FCSQLEIQKLQRVVRQKERQLADAKQCVQFVEAAAHESEQQKEASWKHNQELRKALQQ | | |
| | LQEELQNKSQQLRAWEAEKYNEIRTQEQNIQHLNHSLSHKEQLLQEFRELLQYRDNSD | | |
| | KTLEANEMLLEKLRQRIHDKAVALERAIDEKFSALEEKEKELRQLRLAVRERDHDLER | | |
| | LRDVLSSNEATMQSMESLLRAKGLEVEQLSTTCQNLQWLKEEMETKFSRWQKEQESII | | |
| | QQLQTSLHDRNKEVEDLSATLLCKLGPGQSEIAEELCQRLQRKERMLQDLLSDRNKQV | | |
| | LEHEMEIQGLLQSVSTREQESQAAAEKLVQALMERNSELQALRQYLGGRDSLMSQAPI | | |
| | SNQQAEVTPTGRLGKQTDQGSMQIPSRDDSTSLTAKEDVSIPRSTLGDLDTVAGLEKE | | |
| | LSNAKEELELMAKKERESQMELSALQSMMAVQEEELQVQAADMESLTRNIQIKEDLIK | | |
| | DLQMQLVDPEDIPAMERLTQEVLLLREKVASVESQGQEISGNRRQQQLLLMLEGLVDE | | |
| | RSRLNEALQAERQLYSSLVKFHAHPESSERDRTLQVELEGAQVLRSRLEEVLGRSLER | | |
| | LNRLETLAAIGGAAAGDDTEDTSTEFTDSIEEEAAHHSHQQLVKVALEKSLATVETQN | | |
| | PSFSPPSPMGGDSNRCLQEEMLHLRAEIHQHLEEKRKAEEELKELKAQIEEAGFSSVS | | |
| | HIRNTMLSLCLENAELKEQMGETMSDGWEIEEDKEKGEVMVETVVTKEGLSESSLQAE | | |
| | FRKLQGKLKNAHNIINLLKEQLVLSSKEGNSKLTPELLVHLTSTIERINTELVGSPGK | | |
| | HQHQEEGNVTVRPFPRPQSLDLGATFTVDAHQQLDNQSQPRDPGPQPAFSLPGSTQHL | | |
| | RSQLSQCKQRYQDLQEKLLLSEATVFAQANELEKYRVMLSESLVKQDSKQIQVDFQDL | | |
| | GYETCGRSENEAEREETTSPECEEHNSLKEMVLMEGLCSEQGRRGSTLASSSERKPLE | | |
| | NQLGKQEEFRVYGKSENILVLRKDIEDLKAQLQNANKVIQNLKSRVRSLSVTSDYSSS | | |
| | LERPRKLRAVGTLEGSSPHSVPDEDEGWLSDGTGAFYSPGLQAKKDLESLIQRVSQLE | | |
| | AQLPENGLEEKLAEELRSASWPGKYDSLIQDQARELSYLRQKIREGRGICYLITQHAK | | |
| | DTVKSFEDLLRSNDIDYYLGQSFREQLAQGSQLTERLTSKLSTEDHKSEKDQAGLEPL | | |
| | ALRLSRELQEKEKVIEVLQAKLDARSLTPSSSRALSDSHRSPSSTSFLSDELEACSDM | | |
| | DIVSEYTHYEEKKASPSHSGSSASQGAKAESNSNPISLPTPQNTPKEANQAHSGFHFH | | |
| | SIPKLASLPQAPLPSAPSSFLPFSPTGPPLLGCCETPEVSLAESQQELQMLQKQLGES | | |
| | STVPPASTATLLSNDLEADSSYYLNSAQPHSPPRGTIELGRILEPGYLGSSGKWDVMR | | |
| | PQKGSVSGDLSSGSSVYQLNSKPTGADLLEEHLGEIWNLRQRLEESICINDCLREQLE | | |
| | HRLTSTARGRGSTSNFYSQGLESIPQLCNENRVLREENRRLQAQLSHVSRGHSQETES | | |
| | LREALLSSRSHLQELEKELEHQKVERQQLLEDLREDQQEVLHFREERLSLQENDSRLQ | | |
| | HKLVLLQQQCEEKQQLFESLQSELQIYEALYGNSKKGLKAYSLDACHQIPLSSDLSHL | | |
| | VAEVQALRGQLEQSIQGNNCLRLQLQQQLESGAGKASLSPSSINQNFPASTDPGNKQL | | |
| | LLQGSAVSPPVRDVGMNSPALVFPSSASSTPGSDSVVLSFSFSGLGLDTSPVMKTPPK | | |
| | LEGDATDGSFANKHGRHVIGHIDDYSALRQQIAEGKLLVKKIVSLVRSACSFPGLEAQ | | |
| | GTEGSKGIHELRSSTSALHHALEESASLLTMFWRAALPSTHIPVLPGKQGESTERELL | | |
| | ELRTKVSKQEQLLQSTTEHLKNANQQKESMEQFIVSVTRTHDVLKKARTNLEVKSLRA | | |
| | LPCTPAL | | |
| | SEQ ID NO:295 | 7084 bp | |
| NOV103c, | GTTGAGGGGGCAATCGGGCACGCTCCTCCCCATGGGTTGCCCATCATGTCTAATGGAT | | |
| CG59773-03 DNA Sequence | | | |
| | ATCGCACTCTGTCCCAGCACCTCAATGACCTGAAGAAGGAGAACTTCAGCCTCAAGCT | | |
| | GCTCATCTACTTCCTGGAGGAGCGCATGCAACAGAAGTATGAGGCCAGCCGGGAGGAC | | |
| | ATCTACAAGCGGGGGTGATGTGGAGAATCTCAACAGTCAGAATGAAGCTGAGCTCCGA | | |
| | CGCCAGTTTGAGGAGCGACAGCAGGAGACGGAGCATGTTTATGAGCTCTTGGAGAATA | | |
| | AGATCCAGCTTCTGCAGGAGGAATCCAGGCTAGCAAAGAATGAAGCTGCGCGGATGGC | | |
| | AGCTCTGGTGGAAGCAGAGAAGGAGTGTAACCTGGAGCTCTCAGAGAAACTGAAGGGA | | |
| | GTCACCAAAAACTGGGAAGATGTACCAGGAGACCAGGTCAAGCCCGACCAATACACTG | | |
| | AGACCCTGGCCCAGAGGGACAAGAGAATTGAAGAACTGAATCAGAGCCTGGCTGCCCA | | |
| | GGAGAGGCTTGTAGAACAGCTATCTCGGGAGAAACAACAACTGCTACATCTGTTGGAG | | |
| | GAGCCAACTAGCATGGAAGTGCAGCCCATGACTGAAGAGTTGCTGAAACAACAAAAGC | | |
| | TGAATTCACATGAGACCACTATAACTCAGCAGTCTGTATCTGATTCCCACTTGGCAGA | | |
| | ACTCCAGGAAAAAATCCAGCAAACAGAGGCCACCAACAAGATTCTTCAAGAGAAACTT | | |
| | AATGAAATGAGCTATGAACTAAAGTGTGCTCAGGAGTCGTCTCAAAAGCAAGATGGTA | | |
| | CAATTCAGAACCTCAAGGAAACTCTGAAAAGCAGGGAACGTGAGACTGAGGAGTTGTA | | |
| | CCAGGTAATTGAAGGTCAAAATGACACAATGGCAAAGCTTCGAGAAATGCTGCACCAA | | |
| | AGCCAGCTTGGACAACTTCAGAGCTCAGAGGGTACTTCTCCAGCTCAGCAACAGGTAG | | |
| | CTCTGCTTGATCTTCAGAGTGCTTTATTCTGCAGCCAACTTGAAATACAGAAGCTCCA | | |
| | GAGGGTGGTACGACAGAAAGAGCGCCAACTGGCTGATGCCAAACAATGTGTGCAATTT | | |
| | GTAGAGGCTGCAGCACACGAGAGTGAACAGCAGAAAGAGGCTTCTTGGAAACATAACC | | |
| | AGGAATTGCGAAAAGCCTTGCAGCAGCTACAAGAAGAATTGCAGAATAAGAGCCAACA | | |
| | GCTTCGTGCCTGGGAGGCTGAAAAATACAATGAGATTCGAACCCAGGAACAAAACATC | | |
| | CAGCACCTAAACCATAGTCTGAGTCACAAGGAGCAGTTGCTTCAGGAATTTCGGGAGC | | |
| | TCCTACAGTATCGAGATAACTCAGACAAAACCCTTGAAGCAAATGAAATGTTGCTTGA | | |
| | GAAACTTCGCCAGCGAATACATGATAAAGCTGTTGCTCTGGAGCGGGCTATAGATGAA | | |
| | AAATTCTCTGCTCTAGAAGAGAAAGAAAAAGAACTGCGCCAGCTTCGTCTTGCTGTGA | | |
| | GAGAGCGAGATCATGACTTAGAGAGACTGCGCGATGTCCTCTCCTCCAATGAAGCTAC | | |
| | TATGCAAAGTATGGAGAGTCTCCTGAGGGCCAAAGGCCTGGAAGTGGAACAGTTATCT | | |
| | ACTACCTGTCAAAACCTCCAGTGGCTGAAAGAAGAAATGGAAACCAAATTTAGCCGTT | | |
| | GGCAGAAGGAACAAGAGAGTATCATTCAGCAGTTACAGACGTCTCTTCATGATAGGAA | | |
| | CAAAGAAGTGGAGGATCTTAGTGCAACACTGCTCTGCAAACTTGGACCAGGGCAGAGT | | |
| | GAGATAGCAGAGGAGCTGTGCCAGCGTCTACAGCGAAAGGAAAGGATGCTGCAGGACC | | |
| | TTCTAAGTGATCGAAATAAACAAGTGCTGGAACATGAAATGGAGATTCAAGGCCTGCT | | |
| | TCAGTCTGTGAGCACCAGGGAGCAGGAAAGCCAAGCTGCTGCAGAGAAGTTGGTGCAA | | |
| | GCCTTAATGGAAAGAAATTCAGAATTACAGGCCCTGCGCCAATATTTAGGAGGGAGAG | | |
| | ACTCCCTGATGTCCCAAGCACCCATCTCTAACCAACAAGCTGAAGTTACCCCCACTGG | | |
| | CCGTCTTGGAAAACAGACTGATCAAGGTTCAATGCAGATACCTTCCAGAGATGATAGC | | |
| | ACTTCATTGACTGCCAAAGAGGATGTCAGCATACCCAGATCCACATTAGGAGATTTGG | | |
| | ACACAGTTGCAGGGCTGGAAAAAGAACTGAGTAATGCCAAAGAGGAACTTGAACTCAT | | |
| | GGCTAAAAAAGAAAGAGAATCACAGATGGAACTTTCTGCTCTACAGTCCATGATGGCT | | |
| | GTGCAGGAAGAAGAGCTGCAGGTGCAGGCTGCTGATATGGAGTCTCTGACCAGGAACA | | |
| | TACAGATTAAAGAAGATCTCATAAAGGACCTGCAAATGCAACTGGTTGATCCTGAAGA | | |
| | CATACCAGCTATGGAACGCCTGACCCAGGAAGTCTTACTTCTTCGGGAAAAAGTTGCT | | |
| | TCAGTAGAATCCCAGGGTCAAGAAATTTCAGGAAACCGAAGACAACAGCAGTTGCTGC | | |
| | TGATGCTAGAAGGACTAGTAGATGAACGGAGTCGGCTCAATGAGGCCTTACAAGCAGA | | |
| | GAGACAGCTCTATAGCAGTCTGGTGAAGTTCCATGCCCATCCAGAGAGCTCTGAGAGA | | |
| | GACCGAACTCTGCAGGTGGAACTGGAAGGGGCTCAGGTGTTACGCAGTCGGCTAGAAG | | |
| | AACTTCTTGGAAGAAGCTTGGAGCGCTTAAACAGGCTGGAGACCCTGGCCGCCATTGG | | |
| | AGGTGCAGCTGCAGGGGATGACACCGAAGATACAAGCACTGAGTTCACTGACAGTATT | | |
| | GAGGAGGAGGCTGCACACCATAGTCACCAGCAACTTGTCAAGGTGGCTTTGGAGAAAA | | |
| | GTCTGGCAACTGTGGAGACCCAGAACCCATCTTTTTCCCCTCCTTCTCCGATGGGAGG | | |
| | GGACAGTAACAGGTGTCTTCAGGAAGAAATGCTCCACCTGAGGGCTGAGATCCACCAG | | |
| | CACTTAGAAGAGAAGAGGAAAGCTGAGGAGGAACTGAAGGAGCTAAAGGCTCAAATTG | | |
| | AGGAAGCAGGATTCTCCTCAGTGTCCCACATCAGGAACACCATGCTGAGCCTTTGCCT | | |
| | TGAGAATGCGGAGCTGAAAGAGCAGATGGGAGAAACAATGTCTGATGGATGGGAGATC | | |
| | GAGGAAGACAAGGAGAAGGGCGAGGTGATGGTTGAGACTGTGGTAACCAAAGAGGGTC | | |
| | TGAGTGAGAGTAGCCTTCAGGCTGAGTTCAGAAAGCTCCAGGGAAAACTGAAGAATGC | | |
| | CCACAATATCATCAACCTCCTCAAAGAACAACTTGTGCTGAGTAGCAAGGAAGGGAAT | | |
| | AGTAAACTTACTCCAGAGCTCCTTGTGCATCTGACCAGCACCATCGAAAGAATAAACA | | |
| | CAGAACTGGTTGGTTCCCCTGGGAAGCACCAACACCAAGAGGAGGGGAATGTGACTGT | | |
| | GAGGCCTTTCCCCAGACCCCAGAGCCTTGACCTTGGGGCTACCTTCACAGTGGATGCC | | |
| | CACCAACAGTTGGATAACCAGTCCCAGCCTCGTGACCCTGGGCCTCAGCCAGCGTTTA | | |
| | GCCTACCAGGGTCCACCCAGCACCTGCGCTCCCAGCTGTCACAATGCAAACAACGCTA | | |
| | TCAAGATCTCCAGGAGAAGCTGCTGCTATCAGAAGCCACTGTCTTTGCTCAGGCTAAC | | |
| | GAGCTGGAGAAATACAGAGTTATGCTTAGTGAATCCTTGGTGAAGCAGGACAGCAAGC | | |
| | AGATCCAGGTGGACTTCCAGGACCTGGGCTATGAGACTTGTGGCCGAAGCGAGAATGA | | |
| | GGCTGAACGGGAGGAAACCACCAGTCCTGAGTGTGAGGAGCACAACAGCCTCAAGGAA | | |
| | ATGGTCCTGATGGAGGGGCTGTGCTCTGAGCAGGGACGCCGGGGCTCAACACTGGCTA | | |
| | GTTCCTCTGAGAGGAAGCCCTTGGAGAACCAGCTAGGGAAGCAGGAAGAGTTCCGGGT | | |
| | ATATGGAAAGTCAGAAAACATCTTGGTCCTACGAAAGGACATCGAAGATCTGAAGGCC | | |
| | CAGCTGCAGAATGCCAACAAGGTCATTCAAAACCTCAAGAGCCGGGTCCGGTCCCTCT | | |
| | CAGTTACAAGTGATTATTCGTCTAGTCTGGAAAGACCCCGGAAGCTGAGAGCTGTTGG | | |
| | CACCTTGGAGGGGTCTTCACCTCATAGTGTCCCTGATGAGGATGAGGGGTGGCTGTCT | | |
| | GATGGCACTGGGGCTTTCTACTCTCCAGGGCTTCAGGCCAAAAAGGACCTGGAGAGTC | | |
| | TCATCCAGAGAGTATCCCAGCTGGAGGCCCAGCTCCCAGAAAATGGACTAGAAGAGAA | | |
| | GCTGGCTGAGGAGCTGAGATCAGCCTCGTGGCCTGGGAAATATGATTCCCTGATTCAG | | |
| | GATCAGGCCCGGGAACTGTCTTACCTACGGCAAAAAATACGAGAAGGGAGAGGTATTT | | |
| | GTTATCTTATCACCCAGCATGCAAAAGATACAGTAAAATCTTTTGAGGATCTCCTAAG | | |
| | GAGCAATGACATTGACTACTACCTGGGACAGAGCTTCCGGGAGCAACTCGCCCAGGGA | | |
| | AGCCAGCTGACAGAGAGGCTCACCAGCAAACTCAGCACAGAGGATCATAAAAGTGAGA | | |
| | AAGATCAAGCTGGACTTGAGCCACTGGCCCTCAGGCTCAGCAGGGAGCTGCAGGAGAA | | |
| | GGAGAAAGTGATTGAAGTCCTGCAGGCCAAGCTGGATGCTCGGTCCCTCACACCCTCC | | |
| | AGCAGCCGTGCCTTGTCTGACTCCCACCGCTCTCCCAGCAGCACCTCTTTCCTGTCTG | | |
| | ATGAGCTGGAAGCCTGCTCTGACATGGACATAGTCAGCGAGTACACACACTATGAAGA | | |
| | GAAGAAAGCTTCTCCCAGTCACTCAGGTAGCAGTGCATCTCAGGGGGCTAAGGCCGAA | | |
| | TCCAACAGCAACCCCATCAGCTTGCCAACTCCCCAGAATACCCCCAAGGAGGCCAACC | | |
| | AAGCCCATTCAGGCTTTCATTTTCACTCCATACCCAAGCTGGCTAGCCTTCCTCAGGC | | |
| | ACCATTGCCCTCAGCTCCATCCAGCTTCCTGCCTTTCAGCCCCACTGGCCCTCCCCTC | | |
| | CTTGGCTGCTGTGAGACACCAGAGGTCTCCTTGGCTGAGTCTCAGCAGGAGCTACAGA | | |
| | TGCTGCAGAAGCAGTTGGGAGAAAGTAGCACTGTTCCTCCTGCTTCCACAGCTACATT | | |
| | GCTGAGCAACGACTTGGAAGCCGACTCTTCCTACTACCTCAACTCTGCCCAGCCTCAC | | |
| | TCTCCTCCAAGGGGCACCATAGAACTGGGAAGAATCCTAGAGCCTGGGTACCTGGGCA | | |
| | GCAGTGGCAAGTGGGATGTGATGAGGCCTCAGAAAGGGAGTGTATCTGGGGACCTATC | | |
| | CTCAGGCTCCTCTGTGTACCAGCTTAACTCCAAACCCACAGGGGCTGACCTGCTGGAA | | |
| | GAGCATCTTGGTGAAATCTGGAACCTGCGCCAGCGCCTGGAGGAGTCCATCTGCATCA | | |
| | ATGACTGCCTACGGGAGCAACTGGAACACCGGCTGACCTCTACTGCTCGTGGAAGGGG | | |
| | ATCCACTTCTAACTTCTACAGTCAGGGCCTGGAGTCCATACCTCAGCTCTGCAATGAG | | |
| | AACAGAGTCCTCAGGGAAGAAAATCGAAGACTTCAGGCTCAACTGAGTCATGTTTCCA | | |
| | GAGGTCACTCCCAGGAAACAGAAAGCCTGAGGGAGGCTCTGCTGTCCTCTCGATCCCA | | |
| | CCTTCAAGAGCTGGAAAAGGAGCTGGAGCACCAGAAGGTGGAAAGGCAGCAGCTTTTG | | |
| | GAAGACTTGAGGGAGAAGCAGCAAGAGGTCTTGCATTTCAGGGAGGAACGTCTTTCCC | | |
| | TCCAGGAAAACGACTCCAGACTGCAGCACAAGCTGGTTCTCCTGCAGCAACAGTGTGA | | |
| | AGAGAAACAGCAGCTCTTTGAGTCCCTCCAGTCAGAGCTACAAATCTACGAGGCACTT | | |
| | TATGGCAATTCCAAGAAGGGGCTGAAAGCTTACAGCCTGGATGCCTGTCACCAAATCC | | |
| | CTTTGAGCAGTGACCTGAGCCACCTGGTGGCAGAGGTACAAGCTCTGAGAGGGCAGCT | | |
| | GGAGCAGAGCATTCAGGGGAACAATTGTCTGCGACTGCAGCTGCAACAGCAGCTGGAG | | |
| | AGCGGTGCTGGCAAAGCCAGCCTCAGCCCCTCCTCCATTAACCAGAACTTCCCAGCCA | | |
| | GCACTGACCCTGGAAACAAGCAGCTGCTCCTCCAAGGTTCAGCTGTGTCCCCTCCAGT | | |
| | CCGGGATGTTGGTATGAATTCCCCAGCTCTGGTCTTCCCCAGCTCTGCTTCCTCTACT | | |
| | CCTGGCTCAGATTCAGTTGTGTTGTCATTTTCTTTTTCAGGCTTGGGTTTGGATACTT | | |
| | CTCCAGTAATGAAGACCCCTCCCAAGCTAGAGGGTGATGCTACTGATGGCTCCTTTGC | | |
| | CAATAAGCATGGCCGCCATGTCATTGGCCACATTGATGACTACAGTGCCCTAAGACAG | | |
| | CAGATTGCGGAGGGCAAGCTGCTGGTCAAAAAGATAGTGTCTCTTGTGAGATCAGCGT | | |
| | GCAGCTTCCCTGGCCTTGAAGCCCAAGGCACAGAGGGCAGCAAAGGCATTCATGAGCT | | |
| | TCGGAGCAGCACCAGTGCCCTGCACCATGCCCTAGAGGAGTCGGCTTCCCTCCTCACC | | |
| | ATGTTCTGGAGAGCGGCCCTGCCAAGCACCCACATCCCTGTGCTGCCTGGCAAACAGG | | |
| | GAGAATCAACAGAAAGGGAACTTCTGGAACTGAGAACCAAAGTATCCAAACAGGAGCA | | |
| | GCTCCTTCAGAGCACAACTGAGCATCTGAAGAACGCCAACCAGCAGAAGGAGAGCATG | | |
| | GAACAGTTCATTGTCAGCGTAACCAGAACACATGATGTTTTAAAGAAGGCAAGGACTA | | |
| | ACTTAGAGGTGAAATCCCTAAGGGCTCTGCCGTGTACTCCAGCCTTGTGACCCTTGCC | | |
| | TTCCAGGAACCATGCAAGAAGCGCAGCCACCAGAAGTCCTTAAAACAGCAGGAAAGGT | | |
| | GAGCCTGTCCCCCTTTTGTGCAGCTACCTATCTGCTGAGGAGCATCTGGGCCTCATTC | | |
| | CTCCAAGT | | |
| | ORF Start: ATG at 155 | | ORF Stop: TGA at 6950 |
| | SEQ ID NO:296 | 2265 aa | MW at 255081.5 kD |

| | |
|---|---|
| NOV103c, | MRPAGRTSTSGGDVENLNSQNEAELRRQFEERQQETEHVYELLENKIQLLQEESRLAK |
| CG59773-03 Protein | |
| Sequence | NEAARMAALVEAEKECNLELSEKLKGVTKNWEDVPGDQVKPDQYTETLAQRDKRIEEL |
| | NQSLAAQERLVEQLSREKQQLLHLLEEPTSMEVQPMTEELLKQQKLNSHEETTITQQSV |
| | SDSHLAELQEKIQQTEATNKILQEKLNEMSYELKCAQESSQKQDGTIQNLKETLKSRE |
| | RETEELYQVIEGQNDTMAKLREMLHQSQLGQLHSSEGTSPAQQQVALLDLQSALFCSQ |
| | LEIQKLQRVVRQKERQLADAKQCVQFVEAAAHESEQQKEASWKHNQELRKALQQLQEE |
| | LQNKSQQLRAWEAEKYNEIRTQEQNIQHLNHSLSHKEQLLQEFRELLQYRDNSDKTLE |
| | ANEMLLEKLRQRIHDKAVALERAIDEKFSALEEKEKELRQLRLAVRERDHDLERLRDV |
| | LSSNEATMQSMESLLRAKGLEVEQLSTTCQNLQWLKEEMETKFSRWQKEQESIIQQLQ |
| | TSLHDRNKEVEDLSATLLCKLGPGQSEIAEELCQRLQRKERMLQDLLSDRNKQVLEHE |
| | MEIQGLLQSVSTREQESQAAAEKLVQALMERNSELQALRQYLGGRDSLMSQAPISNQQ |
| | AEVTPTGRLGKQTDQGSMQIPSRDDSTSLTAKEDVSIPRSTLGDLDTVAGLEKELSNA |
| | KEELELMAKKERESQMELSALQSMMAVQEEELQVQAADMESLTRNIQIKEDLIKDLQM |
| | QLVDPEDIPAMERLTQEVLLLREKVASVESQGQEISGNRRQQQLLLMLEGLVDERSRL |
| | NEALQAERQLYSSLVKFHAHPESSERDRTLQVELEGAQVLRSRLEEVLGRSLERLNRL |
| | ETLAAIGGAAAGDDTEDTSTEFTDSIEEEAAHHSHQQLVKVALEKSLATVETQNPSFS |
| | PPSPMGGDSNRCLQEEMLHLRAEFHQHLEEKRKAEEELKELKAQIEEAGFSSVSHIRN |
| | TMLSLCLENAELKEQMGEAMSDGWEIEEDKEKGEVMVETVVTKEGLSESSLQAEFRKL |
| | QGKLKNAHNIINLLKEQLVLSSKEGNSKLTPELLVHLTSTIERINTELVGSPGKHQHQ |
| | EEGNVTVRPFPRPQSLDLGATFTVDAHQQLDNQSQPRDPGPQSAFSLPGSTQHLRSQL |
| | SQCKQRYQDLQEKLLLSEATVFAQANELEKYRVMLSESLVKQDSKQIQVDFQDLGYET |
| | CGRSENEAEREETTSPECEEHNSLKEMVLMEGLCSEQGRRGSTLASSSERKPLENQLG |
| | KQEEFRVYGKSENILVLRKDIEDLKAQLQNANKVIQNLKSRVRSLSVTSDYSSSLERP |
| | RKLRAVGTLEGSSPHSVPDEDEGWLSDGTGAFYSPGLQAKKDLESLIQRVSQLEAQLP |
| | ENGLEEKLAEELRSASWPGKYDSLIQDQARELSYLRQKIREGRGICYLITQHAKDTVK |
| | SFEDLLRSNDIDYYLGQSFREQLAQGSQLTERLTSKLSTEDHKSEKDQAGLEPLALRL |
| | SRELQEKEKVIEVLQAKLDARSLTPSSSRALSDSHRSPSSTSFLSDELEACSDMDIVS |
| | EYTHYEEKKASPSHSGSSASQGAKAESNSNPISLPTPQNTPKEANQAHSGFHFHSIPK |
| | LASLPQAPLPSAPSSFLPFSPTGPPLLGCCETPEVSLAESQQELQMLQKQLGESSTVP |
| | PASTATLLSNDLEADSSYYLNSAQPHSPPRGTIELGRILEPGYLGSSGKWDVMRPQKG |
| | SVSGDLSSGSSVYQLNSKPTGADLLEEHLGEIWNLRQRLEESICINDCLREQLEHRLT |
| | STARGRGSTSNFYSQGLESIPQLCNENRVLREENRRLQAQLSHVSRGHSQETESLREA |
| | LLSSRSHLQELEKELEHQKVERQQLLEDLREKQQEVLHFREERLSLQENDSRLQHKLV |
| | LLQQQCEEKQQLFESLQSELQIYEALYGNSKKGLKAYSLDACHQIPLSSDLSHLVAEV |
| | QALRGQLEQSIQGNNCLRLQLQQQLESGAGKASLSPSSINQNFPASTDPGNKQLLLQG |
| | SAVSPPVRDVGMNSPALVFPSSASSTPGSDSVVLSFSFSGLGLDTSPVKMTPPKLEGD |
| | ATDGSFANKHGRHVIGHIDDYSALRQQIAEGKLLVKKIVSLVRSACSFPGLEAQGTEG |
| | SKGIHELRSSTSALHHALEESASLLTMFWRAALPSTHIPVLPGKQGESTERELLELRT |
| | KVSKQEQLLQSTTEHLKNANQQKESMEQFIVSVTRTHDVLKKARTNLEVKSLRARPCT |
| | PAL |

Sequence comparison of the above protein sequences yields the following sequence relationships shown in Table 103B.

TABLE 103B
Comparison of NOV103a against NOV103b through NOV103c.

| | | Identities/ |
|---|---|---|
| | | Similarities |
| Protein | NOV103a Residues/ | for the |
| Sequence | Match Residues | Matched Region |
| NOV103b | 365 . . . 2196 | 1510/1834 (82%) |
| | 202 . . . 2016 | 1518/1834 (82%) |
| NOV103c | 365 . . . 2196 | 1510/1834 (82%) |
| | 140 . . . 1954 | 1518/1834 (82%) |

Further analysis of the NOV103a protein yielded the following properties shown in Table 103C.

TABLE 103C
Protein Sequence Properties NOV103a

| | |
|---|---|
| PSort | 0.5855 probability located in mitochondrial matrix space; |
| analysis: | 0.4200 probability located in nucleus; 0.3000 probability |
| | located in microbody (peroxisome); 0.2957 probability located |
| | in mitochondrial inner membrane |
| SignalP | Likely cleavage site between residues 39 and 40 |
| analysis: | |

A search of the NOV103a protein against the Geneseq database, a proprietary database that contains sequences published in patents and patent publication, yielded several homologous proteins shown in Table 103D.

TABLE 103D
Geneseq Results for NOV103a

| | | NOV103a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAY71159 | Human phosphodiesterase | 1 . . . 2196 | 2193/2204 (99%) | 0.0 |
| | interacting protein, myomegalin- | 1 . . . 2204 | 2193/2204 (99%) | |
| | Homo sapiens, 2517 aa. | | | |
| | [WO200027861-A1, | | | |
| | 18 MAY 2000] | | | |
| AAM40183 | Human polypeptide SEQ ID NO | 635 . . . 2196 | 1557/1570 (99%) | 0.0 |
| | 3328-Homo sapiens, 1883 aa. | 1 . . . 1570 | 1559/1570 (99%) | |
| | [WO200153312-A1, | | | |
| | 26 JUL. 2001] | | | |
| AAY71158 | phosphodiesterase interacting | 365 . . . 2197 | 1433/1837 (78%) | 0.0 |
| | protein, myomegalin-Rattus sp, | 202 . . . 2017 | 1572/1837 (85%) | |
| | 2326 aa. [WO200027861-A1, | | | |
| | 18 MAY 2000] | | | |
| AAY67600 | Human adipose tissue protein #3- | 1 . . . 934 | 925/934 (99%) | 0.0 |
| | Homo sapiens, 944 aa. | 1 . . . 934 | 927/934 (99%) | |
| | [JP2000037190-A, 8 FEB. 2000] | | | |
| AAU01768 | Human secreted protein #47- | 365 . . . 1102 | 730/738 (98%) | 0.0 |
| | Homo sapiens, 934 aa. | 197 . . . 934 | 733/738 (98%) | |
| | [WO200123546-A1, 5 APR. 2001] | | | |

In a BLAST search of public sequence databases, the NOV103a protein was found to have homology to the proteins shown in the BLASTP data in Table 103E.

TABLE 103E
Public BLASTP Results for NOV103a

| | | NOV103a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O75042 | KIAA0454 PROTEIN-Homo | 636 . . . 2196 | 1558/1569 (99%) | 0.0 |
| | sapiens (Human), 1882 aa | 1 . . . 1569 | 1558/1569 (99%) | |
| | (fragment). | | | |
| Q9WUJ3 | MYOMEGALIN-Rattus | 365 . . . 2197 | 1444/1838 (78%) | 0.0 |
| | norvegicus (Rat), 2324 aa. | 202 . . . 2015 | 1581/1838 (85%) | |
| O75065 | KIAA0477 PROTEIN-Homo | 1 . . . 1132 | 1132/1132 (100%) | 0.0 |
| | sapiens (Human), 1132 aa. | 1 . . . 1132 | 1132/1132 (100%) | |
| Q25893 | LIVER STAGE ANTIGEN- | 356 . . . 1459 | 243/1129 (21%) | 4e−35 |
| | Plasmodium falciparum (isolate | 605 . . . 1651 | 488/1129 (42%) | |
| | NF54), 1909 aa. | | | |
| Q13439 | Golgi autoantigen, golgin | 229 . . . 1749 | 349/1638 (21%) | 4e−34 |
| | subfamily A 4 (Trans-Golgi p230) | 267 . . . 1814 | 679/1638 (41%) | |
| | (256 kDa golgin) (Golgin-245) | | | |
| | (72.1 protein)-Homo sapiens | | | |
| | (Human), 2230 aa. | | | |

PFam analysis predicts that the NOV103a protein contains the domains shown in the Table 103F.

TABLE 103F
Domain Analysis of NOV103a

| | | Identities/ | |
|---|---|---|---|
| Pfam | NOV103a | Similarities for | Expect |
| Domain | Match Region | the Matched Region | Value |
| Somatomedin_B: | 150 . . . 189 | 14/47 (30%) | 7.6 |
| domain 1 of 1 | | 25/47 (53%) | |
| necA: domain 1 of 1 | 621 . . . 650 | 8/30 (27%) | 8.1 |
| | | 22/30 (73%) | |
| Ribosomal_L10: | 604 . . . 695 | 20/109 (18%) | 9.9 |
| domain 1 of 1 | | 59/109 (54%) | |
| Dishevelled: | 844 . . . 914 | 19/74 (26%) | 2.7 |
| domain 1 of 1 | | 37/74 (50%) | |
| Transposase_22: | 1135 . . . 1416 | 71/376 (19%) | 4.6 |
| domain 1 of 1 | | 127/376 (34%) | |
| Phe_tRNA-synt_N: | 2079 . . . 2152 | 13/79 (16%) | 4.9 |
| domain 1 of 1 | | 49/79 (62%) | |

### Example 104

The NOV104 clone was analyzed, and the nucleotide and predicted polypeptide sequences are shown in Table 104A.

TABLE 104A
NOV104 Sequence Analysis

| | SEQ ID NO:297 | 736 bp | |
|---|---|---|---|
| NOV104a, | AAAGCACCCGAGATGACCCCGGCTCCTCCACCAGGAGCGCGGCCGGGCGCGGCGTCCC | | |
| CG57460-01 DNA Sequence | | | |
| | TAGCGGGCTTCGCCGGGGTGGCGTCTCTGGGGCCTGGGGACCCCCGCCGCGCCGCTGA | | |
| | CCCGCGCCCTCTGCCCCCAGCGCTGTGCTTCGCCGTGAGCCGCTCGCTGCTGCTGACG | | |
| | TGCCTGGTGCCGGCCGCGCTGCTGGGCCTGCGCTACTACTACAGCCGCAAGGTGATCC | | |
| | GCGCCTACCTGGAGTGCGCGCTGCACACGGACATGGCGGACATCGAGCAGTACTACAT | | |
| | GAAGCCGCCCGGTGTGTCCCTGACCGCCCTATCCCCTGCAGGCTCCTGCTTCTGGGTG | | |
| | GCCGTGCTGGATGGCAACGTGGTGGGCATTGTGGCTGCACGGGCCCACGAGGAGGACA | | |
| | ACACGGTGGAGCTGCTGCGGATGTCTGTGGACTCACGTTTCCGAGGCAAGGGCATCGC | | |
| | CAAGGCGCTGGGCCGGAAGGTGCTGGAGTTCGCCGTGGTGCACAACTACTCCGCGGTG | | |
| | GTGCTGGGCACGACGGCCGTCAAGGTGGCCGCCCACAAGCTCTACGAGTCGCTGGGCT | | |
| | TCAGACACATGGGCGCCAGTGACCACTACGTGCTGCCGGGCATGACCCTCTCGCTGGC | | |
| | TGAGCGCCTCTTCTTCCAGGTCCGCTACCACCGCTACCGCCTGCAGCTGCGCGAGGAG | | |
| | TGACCGCCGCCGCTCGCCCGCCCGCCCCCCCGGCCGCCCT | | |
| | ORF Start: ATG at 13 | | ORF Stop: TGA at 697 |
| | SEQ ID NO:298 | 228 aa | MW at 24767.5 kD |

| | |
|---|---|
| NOV104a, | MTPAPPPGARPGAASLAGFAGVASLGPGDPRRAADPRPLPPALCFAVSRSLLLTCLVP |
| CG57460-01 Protein Sequence | |
| | AALLGLRYYYSRKVIRAYLECALHTDMADIEQYYMKPPGVSLTALSPAGSCFWVAVLD |
| | GNVVGIVAARAHEEDNTVELLRMSVDSRFRGKGIAKALGRKVLEFAVVHNYSAVVLGT |
| | TAVKVAAHKLYESLGFRHMGASDHYVLPGMTLSLAERLFFQVRYHRYRLQLREE |

Further analysis of the NOV104a protein yielded the following properties shown in Table 104B.

TABLE 104B
Protein Sequence Properties NOV104a

| | |
|---|---|
| PSort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic reticulum (lumen) |
| SignalP | Likely cleavage site between residues 64 and 65 |
| analysis: | |

A search of the NOV104a protein against the Geneseq database, a proprietary database that contains sequences published in patents and patent publication, yielded several homologous proteins shown in Table 104C.

TABLE 104C
Geneseq Results for NOV104a

| | | NOV104a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB19986 | Human camello 3 (Hcml3) protein | 42 . . . 195 | 144/154 (93%) | 7e−76 |
| | (partial)-Homo sapiens, 144 aa. | 1 . . . 144 | 144/154 (93%) | |
| | [WO200077024-A1, | | | |
| | 21 DEC. 2000] | | | |
| AAB19985 | Human camello 2 (Hcml2) protein- | 47 . . . 200 | 63/158 (39%) | 1e−21 |
| | Homo sapiens, 227 aa. | 56 . . . 203 | 92/158 (57%) | |
| | [WO200077024-A1, | | | |
| | 21 DEC. 2000] | | | |
| AAB19984 | Human camello 1 (Hcml1) protein- | 41 . . . 196 | 60/160 (37%) | 7e−20 |
| | Homo sapiens, 227 aa. | 50 . . . 199 | 88/160 (54%) | |
| | [WO200077024-A1, | | | |
| | 21 DEC. 2000] | | | |
| AAY57959 | Human TSC501 protein SEQ ID | 41 . . . 196 | 59/160 (36%) | 4e−19 |
| | NO: 1-Homo sapiens, 227 aa. | 50 . . . 199 | 87/160 (53%) | |
| | [JP11332579-A, 7 DEC. 1999] | | | |
| AAB19987 | Mouse camello 1 (Mcml1) | 41 . . . 194 | 63/158 (39%) | 1e−18 |
| | protein-Mus sp, 222 aa. | 50 . . . 197 | 87/158 (54%) | |
| | [WO200077024-A1, | | | |
| | 21 DEC. 2000] | | | |

In a BLAST search of public sequence databases, the NOV104a protein was found to have homology to the proteins shown in the BLASTP data in Table 104D.

TABLE 104D
Public BLASTP Results for NOV104a

| | | NOV104a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9UHF3 | PUTATIVE N- | 47 . . . 200 | 63/158 (39%) | 5e−21 |
| | ACETYLTRANSFERASE | 56 . . . 203 | 92/158 (57%) | |
| | CAMELLO 2-Homo sapiens | | | |
| | (Human), 227 aa. | | | |
| Q9UHE5 | PUTATIVE N- | 41 . . . 196 | 60/160 (37%) | 3e−19 |
| | ACETYLTRANSFERASE CML1- | 50 . . . 199 | 88/160 (54%) | |
| | Homo sapiens (Human), 227 aa. | | | |
| Q9UQ17 | GLA PROTEIN-Homo sapiens | 41 . . . 196 | 60/160 (37%) | 3e−19 |
| | (Human), 227 aa. | 50 . . . 199 | 88/160 (54%) | |
| Q96QI8 | KIDNEY-AND LIVER-SPECIFIC | 41 . . . 196 | 59/160 (36%) | 1e−18 |
| | GENE -Homo sapiens (Human), | 50 . . . 199 | 87/160 (53%) | |
| | 227 aa. | | | |
| O75839 | TSC501 PROTEIN-Homo | 41 . . . 196 | 59/160 (36%) | 1e−18 |
| | sapiens (Human), 227 aa. | 50 . . . 199 | 87/160 (53%) | |

PFam analysis predicts that the NOV104a protein contains the domains shown in the Table 104E.

TABLE 104E
Domain Analysis of NOV104a

| | | Identities/ | |
|---|---|---|---|
| Pfam | NOV104a | Similarities for | Expect |
| Domain | Match Region | the Matched Region | Value |
| Acetyltransf: domain 1 | 111 . . . 191 | 28/82 (34%) | 2.2e−17 |
| of 1 | | 64/82 (78%) | |

### Example 105

The NOV105 clone was analyzed, and the nucleotide and predicted polypeptide sequences are shown in Table 105A.

TABLE 105A
NOV105 Sequence Analysis

| | SEQ ID NO:299 | 1230 bp | |
|---|---|---|---|
| NOV105a, | CTTCCCGGCGGCTGCGCGATGGACAGCCCCGAGGTGACCTTCACTCTCGCCTATCTGG | | |
| CG57464-01 DNA Sequence | | | |
| | TGTTCGCCGTGTGCTTCGTGTTCACGCCCAACGAGTTCCACGCGGCGGGGCTCACGGT | | |
| | GCAGAACCTGCTGTCGGGCTGGCTGGGCAGCGAGGACGCCGCCTTCGTGCCCTTCCAC | | |
| | TTGCGCCGCACGGCCGCCACGCTGTTGTGCCACTCGCTGCTGCCGCTCGGTGAGGCTG | | |
| | CTCGGGCCGGCCGGCCGCATCCTCTCCTGCGCAGGGCTTGCTGGGAGGTCAGGAGGAG | | |
| | GCCTCCGCCAGCTCCCCGAGGCCCCGAAAGCGCCTGGGCGCAGCTGGGGAGAGGCGCC | | |
| | GGTCCTCATCCAGAGGGACCGCGGCGTGGGCTGAGCGCGCTTAGGGGTGCCGCCGGCC | | |
| | TGGCCTGGCGGCTCTTCCTGCTGCTGGCCGTGACCCTCCCCTCCATCGCCTGCATCCT | | |
| | GATCTACTACTGGTCCCGTGACCGGTGGGCCTGCCACCCACTGGCGCGCACCCTGGCC | | |
| | CTCTACGCCCTCCCACAGTCTGGCTGGCAGGCTGTTGCCTCCTCTGTCAACACTGAGT | | |
| | TCCGGCGGATTGACAAGTTTGCCACCGGTGCACCAGGTGCCCGTGTGATTGTGACAGA | | |
| | CACGTGGGTGATGAAGGTAACCACCTACCGAGTGCACGTGGCCCAGCAGCAGGACGTG | | |
| | CACCTGACTGTGACGGAGTCTCGGCAGCATGAGCTCTCGCCAGACTCGAACTTGCCCG | | |
| | TGCAGCTCCTCACCATCCGTGTGGCCAGCACCAACCCTGCTGTGCAGGCCTTTGACAT | | |
| | CAGGCTGAACTCCACTGAGTACGGGGAGCTCTGCGAGAAGCTCCGGGCACCCATCCGC | | |
| | AGGGCAGCCCATGTGGTCATCCACCAGAGCCTGGGCGACCTGTTCCTGGAGACATTTG | | |
| | CCTCCCTGGTAGAGGTCAACCCGGCCTACTCAGTGCCCAGCAGCCAGGTGGGGGGCCT | | |
| | GGAGGCCTGCATAGGCTGCATGCAGACACGTGCCAGCGTGAAGCTGGTGAAGACCTGC | | |
| | CAGGAGGCAGCCACAGGCGAGTGCCAGCAGTGTTACTGCCGCCCCATGTGGTGCCTCA | | |
| | CCTGCATGGGCAAGTGGTTCGCCAGCCGCCAGGACCCCCTGCGCCCTGACACCTGGCT | | |
| | GGCCAGCCGCGTGCCCTGCCCCACCTGCCGCGCACGCTTCTGCATCCTGGATGTGTGC | | |
| | ACCGTGCGCTGA | | |
| | ORF Start: ATG at 19 | | ORF Stop: TGA at 1228 |
| | SEQ ID NO:300 | 403 aa | MW at 44585.0 kD |

| | |
|---|---|
| NOV105a, | MDSPEVTFTLAYLVFAVCFVFTPNEFHAAGLTVQNLLSGWLGSEDAAFVPFHLRRTAA |
| CG57464-01 Protein Sequence | |
| | TLLCHSLLPLGEAARAGRPHPLLRRACWEVRRRPPPAPRGPESAWAQLGRGAGPHPEG |
| | PRRGLSALRGAAGLAWRLFLLLAVTLPSIACILIYYWSRDRWACHPLARTLALYALPQ |
| | SGWQAVASSVNTEFRRIDKFATGAPGARVIVTDTWVMKVTTYRVHVAQQQDVHLTVTE |
| | SRQHELSPDSNLPVQLLTIRVASTNPAVQAFDIRLNSTEYGELCEKLRAPIRRAAHVV |
| | IHQSLGDLFLETFASLVEVNPAYSVPSSQVGGLEACIGCMQTRASVKLVKTCQEAATG |
| | ECQQCYCRPMWCLTCMGKWFASRQDPLRPDTWLASRVPCPTCRARFCILDVCTVR |

Further analysis of the NOV105a protein yielded the following properties shown in Table 105B.

TABLE 105B
Protein Sequence Properties NOV105a

| | |
|---|---|
| PSort | 0.6760 probability located in plasma membrane; 0.1000 |
| analysis: | probability located in endoplasmic reticulum (membrane); |
| | 0.1000 probability located in endoplasmic reticulum (lumen); |
| | 0.1000 probability located in outside |
| SignalP | Likely cleavage site between residues 29 and 30 |
| analysis: | |

A search of the NOV105a protein against the Geneseq database, a proprietary database that contains sequences published in patents and patent publication, yielded several homologous proteins shown in Table 105C.

TABLE 105C
Geneseq Results for NOV105a

| | | NOV105a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAG81377 | Human AFP protein sequence SEQ | 1 . . . 403 | 344/409 (84%) | 0.0 |
| | ID NO: 272-Homo sapiens, 362 | 1 . . . 362 | 345/409 (84%) | |
| | aa. [WO200129221-A2, | | | |
| | 26 APR. 2001] | | | |

In a BLAST search of public sequence databases, the NOV105a protein was found to have homology to the proteins shown in the BLASTP data in Table 105D.

TABLE 105D
Public BLASTP Results for NOV105a

| | | NOV105a | | |
|---|---|---|---|---|
| Protein | | Residues/ | Identities/ | |
| Accession | | Match | Similarities for | Expect |
| Number | Protein/Organism/Length | Residues | the Matched Portion | Value |
| CAC38627 | SEQUENCE 271 FROM | 1 . . . 403 | 345/409 (84%) | 0.0 |
| | PATENT WO0129221-Homo | 1 . . . 362 | 346/409 (84%) | |
| | sapiens (Human), 362 aa. | | | |
| Q9DCF3 | 0610039G24RIK PROTEIN- | 1 . . . 403 | 311/403 (77%) | e−176 |
| | Mus musculus (Mouse), 362 aa. | 1 . . . 362 | 328/403 (81%) | |
| Q96GP5 | SIMILAR TO RIKEN CDNA | 1 . . . 265 | 211/271 (77%) | e−109 |
| | 0610039G24 GENE-Homo | 1 . . . 226 | 212/271 (77%) | |
| | sapiens (Human), 232 aa. | | | |
| Q9VN16 | CG14646 PROTEIN-Drosophila | 1 . . . 399 | 123/409 (30%) | 1e−55 |
| | melanogaster (Fruit fly), 409 aa. | 1 . . . 383 | 202/409 (49%) | |
| Q95TM4 | LD39811P-Drosophila | 20 . . . 399 | 117/390 (30%) | 1e−51 |
| | melanogaster (Fruit fly), 393 aa. | 4 . . . 367 | 192/390 (49%) | |

PFam analysis predicts that the NOV105a protein contains the domains shown in the Table 105E.

TABLE 105E
Domain Analysis of NOV105a

| | | | |
|---|---|---|---|
| | | Identities/ | |
| Pfam | NOV105a | Similarities for | Expect |
| Domain | Match Region | the Matched Region | Value |

No Significant Matches Found

### Example 106

The NOV106 clone was analyzed, and the nucleotide and predicted polypeptide sequences are shown in Table 106A.

TABLE 106A
NOV106 Sequence Analysis

| | SEQ ID NO:301 | 1136 bp | |
|---|---|---|---|
| NOV106a, | TTTCTGCAATGGGAGCCTCCGCCACCCACCCTGGAGCCACAGAAGGCCCAGAAGCCAA | | |
| CG57466-01 DNA Sequence | | | |
| | ATGGACAGCTGGTGAACCCCAACAACTTCTGGAAGAACCCGAAAGATGTGTGCGCCCA | | |
| | CGCCCATGGCCTCTCAGGGCCCAGGCCTGGGACGTGACCACCACTAACTGCTCAGCCA | | |
| | ATATCAACTTGACCCACCAGCCCTGGTTCCAGGTCCTGGAGCCGCAGTTCCGGCAGTT | | |
| | TCTCTTCTACCGCCACTGCCGCTACTTCCCCATGCTGCTGAACCACCCGGAGAAGTGC | | |
| | AGGGGCGATGTCTACCTGCTGGTGGTTGTCAAGTCGGTCATCACGCAGCACGACCGCC | | |
| | GCGAGGCCATCCGCCAGACCTGGGCGCGAGCGGCAGTCCGCGGGTGGGGGCCGAGCGC | | |
| | CGTGCGCACCCTCTTCCTGCTGGGCACGGCCTCCAAGCAGGAGGAGCGCACGCACTAC | | |
| | CAGCAGCTGCTGGCCTACGAAGACGCCCTCTACGGCGACATCCTGCAGTGGGGCTTTC | | |
| | TCGACACCTTCTTCAACCTGACCCTCAAGGAGATCCACTTCCTCAAGTGGCTGGACAT | | |
| | CTACTGCCCCCACGTCCCCTTCATTTTCAAAGGCGACGATGACGTCTTCGTCAACCCC | | |
| | ACCAACCTGCTAGAATTTCTGGCTGACCGGCAGCCACAGGAAAACCTGTTCGTGGGCG | | |
| | ATGTCCTGCAGCACGCTCGGCCCATTCGCAGGAAAGACAACAAATACTACATCCCGGG | | |
| | GGCCCTGTACGGCAAGGCCAGCTATCCGCCGTATGCAGGCGGCGGTGGCTTCCTCATG | | |
| | GCCGGCAGCCTGGCCCGGCGCCTGCACCATGCCTGCGACACCCTGGAGCTCTACCCGA | | |
| | TCGACGACGTCTTTCTGGGCATGTGCCTGGAGGTGCTGGGCGTGCAGCCCACGGCCCA | | |
| | CGAGGGCTTCAAGACTTTCGGCATCTCCCGGAACCGCAACAGCCGCATGAACAAGGAG | | |
| | CCGTGCTTTTTCCGCGCCATGCTCGTGGTGCACAAGCTGCTGCCCCCTGAGCTGCTCG | | |
| | CCATGTGGGGGCTGGTGCACAGCAATCTCACCTGCTCCCGCAAGCTCCAGGTGCTCTG | | |
| | ACCCCAGCCGGGCTACTAGGACAGGCCAGGGCAC | | |
| | ORF Start: ATG at 9 | | ORF Stop: TGA at 1101 |
| | SEQ ID NO:302 | 364 aa | MW at 41853.8 kD |

| | |
|---|---|
| NOV106a, | MGASATHPGATEGPEAKWTAGEPQQLLEEPERCVRPRPWPLRAQAWDVTTTNCSANIN |
| CG57466-01 Protein Sequence | |
| | LTHQPWFQVLEPQFRQFLFYRHCRYFPDMLLNHPEKCRGDVYLLVVVKSVITQHDRREA |
| | IRQTWARAAVRGWGPSAVRTLFLLGTASKQEERTHYQQLLAYEDALYGDILQWGFLDT |
| | FFNLTLKEIHFLKWLDIYCPHVPFIFKGDDDVFVNPTNLLEFLADRQPQENLFVGDVL |
| | QHARPIRRKDNKYYIPGALYGKASYPPYAGGGGFLMAGSLARRLHHACDTLELYPIDD |
| | VFLGMCLEVLGVQPTAHEGFKTFGISRNRNSRMNKEPCFFRAMLVVHKLLPPELLAMW |
| | GLVHSNLTCSRKLQVL |

Further analysis of the NOV106a protein yielded the following properties shown in Table 106B.

TABLE 106B
Protein Sequence Properties NOV106a

| | |
|---|---|
| PSort | 0.6400 probability located in microbody (peroxisome); 0.4500 |
| analysis: | probability located in cytoplasm; 0.3122 probability located |
| | in lysosome (lumen); 0.1000 probability located in |
| | mitochondrial matrix space |
| SignalP | No Known Signal Sequence Predicted |
| analysis: | |

A search of the NOV106a protein against the Geneseq database, a proprietary database that contains sequences published in patents and patent publication, yielded several homologous proteins shown in Table 106C.

TABLE 106C
Geneseq Results for NOV106a

| | | NOV106a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB24035 | Human PRO4397 protein sequence | 72 . . . 352 | 149/300 (49%) | 4e−76 |
| | SEQ ID NO: 42-Homo sapiens, | 84 . . . 380 | 191/300 (63%) | |
| | 402 aa. [WO200053750-A1, | | | |
| | 14 SEP. 2000] | | | |
| AAU29167 | Human PRO polypeptide sequence | 26 . . . 363 | 149/348 (42%) | 9e−76 |
| | #144-Homo sapiens, 372 aa. | 27 . . . 371 | 207/348 (58%) | |
| | [WO200168848-A2, | | | |
| | 20 SEP. 2001] | | | |
| AAB88404 | Human membrane or secretory | 26 . . . 363 | 149/348 (42%) | 9e−76 |
| | protein clone PSEC0159-Homo | 27 . . . 371 | 207/348 (58%) | |
| | sapiens, 372 aa. [EP1067182-A2, | | | |
| | 10 JAN. 2001] | | | |
| AAB49750 | Human beta 1,3-N- | 26 . . . 363 | 149/348 (42%) | 9e−76 |
| | acetylglucosamine transferase | 27 . . . 371 | 207/348 (58%) | |
| | protein G4-Homo sapiens, 372 aa. | | | |
| | [WO200100848-A1, 4 JAN. 2001] | | | |
| AAB49749 | Human beta 1,3-N- | 26 . . . 363 | 149/348 (42%) | 9e−76 |
| | acetylglucosamine transferase | 27 . . . 371 | 207/348 (58%) | |
| | protein G4-Homo sapiens, 372 aa. | | | |
| | [WO200100848-A1, 4 JAN. 2001] | | | |

In a BLAST search of public sequence databases, the NOV106a protein was found to have homology to the proteins shown in the BLASTP data in Table 106D.

TABLE 106D
Public BLASTP Results for NOV106a

| | | NOV106a | | |
|---|---|---|---|---|
| Protein | | Residues/ | Identities/ | |
| Accession | | Match | Similarities for | Expect |
| Number | Protein/Organism/Length | Residues | the Matched Portion | Value |
| AAL32295 | BETA-3-GALACTOSYLTRANSFERASE- | 46 . . . 364 | 199/319 (62%) | e−121 |
| | Brachydanio rerio (Zebrafish) (Zebra danjo), | 101 . . . 417 | 249/319 (77%) | |
| | 418 aa. | | | |
| AAL32297 | BETA-3-GALACTOSYLTRANSFERASE- | 29 . . . 360 | 180/337 (53%) | e−104 |
| | Brachydanio rerio (Zebrafish) (Zebra danio), | 82 . . . 409 | 244/337 (71%) | |
| | 412 aa. | | | |
| Q96EK0 | UNKNOWN (PROTEIN FOR MGC: 20513)- | 60 . . . 352 | 152/313 (48%) | 9e−76 |
| | Homo sapiens (Human), 377 aa. | 46 . . . 355 | 198/313 (62%) | |
| CAC39768 | SEQUENCE 175 FROM PATENT | 26 . . . 363 | 149/348 (42%) | 3e−75 |
| | EP1067182-Homo sapiens (Human), 372 aa. | 27 . . . 371 | 207/348 (58%) | |
| Q9C0J2 | BETA-1,3-N- | 26 . . . 363 | 149/348 (42%) | 3e−75 |
| | ACETYLGLUCOSAMINYLTRANSFERASE | 27 . . . 371 | 207/348 (58%) | |
| | BGNT-3-Homo sapiens (Human), 372 aa. | | | |

PFam analysis predicts that the NOV106a protein contains the domains shown in the Table 106E.

TABLE 106E
Domain Analysis of NOV106a

| | | Identities/ | |
|---|---|---|---|
| Pfam | NOV106a | Similarities for | Expect |
| Domain | Match Region | the Matched Region | Value |
| P13_P14_kinase: | 195 . . . 205 | 8/12 (67%) | 8.5 |
| domain 1 of 1 | | 10/12 (83%) | |
| Galactosyl_T: | 112 . . . 308 | 69/212 (33%) | 7.7e−45 |
| domain 1 of 1 | | 148/212 (70%) | |

### Example 107

The NOV107 clone was analyzed, and the nucleotide and predicted polypeptide sequences are shown in Table 107A.

TABLE 107A
NOV107 Sequence Analysis

| | SEQ ID NO:303 | 4091 bp | |
|---|---|---|---|
| NOV 107a, | AAGCAAGAGGCTGAGATGGATCTTGAGGCGGCAAAGAACGGAACAGCCTGGCGCCCCA | | |
| CG57468-01 DNA Sequence | | | |
| | CGAGCGCGGAGGGCGACTTTGAACTGGGCATCAGCAGCAAACAAAAAAGGAAAAAAAC | | |
| | GAAGACAGTGAAAATGATTGGAGTATTAACATTGTTTCGATACTCCGATTGGCAGGAT | | |
| | AAATTGTTTATGTCGCTGGGTACCATCATGGCCATAGCTCACGGATCAGGTCTCCCCC | | |
| | TCATGATGATAGTATTTGGAGAGATGACTGACAAATTTGTTGATACTGCAGGAAACTT | | |
| | CTCCTTTCCAGTGAACTTTTCCTTGTCGCTGCTAAATCCAGGCAAAATTCTGGAAGAA | | |
| | GAAATGACTAGATATGCATATTACTACTCAGGATTGGGTGCTGGAGTTCTTGTTGCTG | | |
| | CCTATATACAAGTTTCATTTTGGACTTTGGCAGCTGGTCGACAGATCAGGAAAATTAG | | |
| | GCAGAAGTTTTTTCATGCTATTCTACGACAGGAAATAGGATGGTTTGACATCAATGAC | | |
| | ACCACTGAACTCAATACGCGGCTAACAGATGACATCTCCAAAATCAGTGAAGGAATTG | | |
| | GTGACAAGGTTGGAATGTTCTTTCAAGCAGTAGCCACGTTTTTTGCAGGATTCATAGT | | |
| | GGGATTCATCAGAGGATGGAAGCTCACCCTTGTGATAATGGCCATCAGCCCTATTCTA | | |
| | GGACTCTCTGCAGCCGTTTGGGCAAAGATACTCTCGGCATTTAGTGACAAAGAACTAG | | |
| | CTGCTTATGCAAAAGCAGGCGCCGTGGCAGAAGAGGCTCTGGGGGCCATCAGGACTGT | | |
| | GATAGCTTTCGGGGGCCAGAACAAAGAGCTGGAAAGGTATCAGAAACATTTAGAAAAT | | |
| | GCCAAAGAGATTGGAATTAAAAAAGCTATTTCAGCAAACATTTCCATGGGTATTGCCT | | |
| | TCCTGTTAATATATGCATCATATGCACTGGCCTTCTGGTATGGATCCACTCTAGTCAT | | |
| | ATCAAAAGAATATACTATTGGAAATGCAATGACAGTTTTTTTTTCAATCCTAATTGGA | | |
| | GCTATGGCCATCGGAGAAACGCTCGTTTTGGCTCCTGAATATTCCAAAGCCAAATCGG | | |
| | GGGCTGCGCATCTGTTTGCCTTGTTGGAAAAGAAACCAAATATAGACAGCCGCAGTCA | | |
| | AGAAGGGAAAAAGCCAGTAAGCGACACATGTGAAGGGAATTTAGAGTTTCGAGAAGTC | | |
| | TCTTTCTTCTATCCATGTCGCCCAGATGTTTTCATCCTCCGTGGCTTATCCCTCAGTA | | |
| | TTGAGCGAGGAAAGACAGTAGCATTTGTGGGGAGCAGCGGCTGTGGGAAAAGCACTTC | | |
| | TGTTCAACTTCTGCAGAGACTTTATGACCCCGTGCAAGGACAAGTGGATGGTGTGGAT | | |
| | GCAAAAGAATTGAATGTACAGTGGCTCCGTTCCCAAATAGCAATCGTTCCTCAAGAGC | | |
| | CTGTGCTCTTCAACTGCAGCATTGCTGAGAACATCGCCTATGGTGACAACAGCCGTGT | | |
| | GGTGCCATTAGATGAGATCAAAGAAGCCGCAAATGCAGCAAATATCCATTCTTTTATT | | |
| | GAAGGTCTCCCTGAGAAATACAACACACAAGTTGGACTGAAAGGAGCACAGCTTTCTG | | |
| | GCGGCCAGAAACAAAGACTAGCTATTGCAAGGGCTCTTCTCCAAAAACCCAAAATTTT | | |
| | ATTGTTGGATGAGGCCACTTCAGCCCTCGATAATGACAGTGAGTGGCAGGTGGTTCAG | | |
| | CATGCCCTTGATAAAGCCAGGACGGGAAGGACATGCCTAGTGGTCACTCACAGGCTCT | | |
| | CTGCAATTCAGAACGCAGATTTGATAGTGGTTCTGCACAATGGAAAGATAAAGGAACA | | |
| | AGGAACTCATCAAGAGCTCCTGAGAAATCGAGACATATATTTTAAGTTAGTGAATGCA | | |
| | CAGTCAGCGAGCAAAGGTCGGACTACAATCGTGGTAGCACACCGACTTTCTACTATTC | | |
| | GAAGTGCAGATTTGATTGTGACCCTAAAGGATGGAATGCTGGCGGAGAAAGGAGCACA | | |
| | TGCTGAACTAATGGCAAAACGAGGTCTATATTATTCACTTGTGATGTCACAGGTAATG | | |
| | CTTATGGGGACTCTTTCAGACTGTGGTAATAGTCTTCCTGAAGTCTCTCTATTAAAAA | | |
| | TTTTAAAGTTAAACAAGCCTGAATGGCCTTTTGTGGTTCTGGGGACATTGGCTTCTGT | | |
| | TCTAAATGGAACTGTTCATCCAGTATTTTCCATCATCTTTGCAAAAATTATAACCGTA | | |
| | ATGTTTGGAAATAATGATCTTTTGTTTTTCCTCAAAATTTTTTTATATTCATTCCTTT | | |
| | TGTTTTTCCTCAAACAAGGTTTCAGCGTAGATTTTTGTTTGTTTGCTTTTCAGGGATT | | |
| | ATTTTACGGCAGAGCAGGGGAAATTTTAACGATGAGATTAAGACACTTGGCCTTCAAA | | |
| | GCCATGTTATATCAGGATATTGCCTGGTTTGATGAAAAGGAAAACAGCACAGGAGGCT | | |
| | TGACAACAATATTAGCCATAGATATAGCACAAATTCAAGGAGCAACAGGTTCCAGGAT | | |
| | TGGCGTCTTAACACAAAATGCAACTAACATGGGACTTTCAGTTATCATTTCCTTTATA | | |
| | TATGGATGGGAGATGACATTCCTGATTCTGAGTATTGCTCCAGTACTTGCCGTGACAG | | |
| | GAATGATTGAAACCGCAGCAATGACTGGATTTGCCAACAAAGATAAGCAAGAACTTAA | | |
| | GCATGCTGGAAAGGTAAAGATAGCAACTGAAGCTTTGGAGAATATACGTACTATAGTG | | |
| | TCATTAACAAGGGAAAAAGCCTTCGAGCAAATGTATGAAGAGATGCTTCAGACTCAAC | | |
| | ACAGGAGAAATACCTCGAAGAAAGCACAGATTATTGGAAGCTGTTATGCATTCAGCCA | | |
| | TGCCTTTATATATTTTGCCTATGCGGCAGGGTTTCGATTTGGAGCCTATTTAATTCAA | | |
| | GCTGGACGAATGTCAAATGCTTTATCTTTTGATAGAGTTTTTACTGCAATTGCATATG | | |
| | GAGCTATGGCCATCGGAGAAACGCTCGTTTTGGCTCCTGAATATTCCAAAGCCAAATC | | |
| | GGGGGCTGCGCATCTGTTTGCCTTGTTGGAAAAGAAACCAAATATAGACAGCCGCAGT | | |
| | CAAGAAGGGAAAAAGCCACTTTCACAGGACACATGTGAAGGGAATTTAGAGTTTCGAG | | |
| | AAGTCTCTTTCTTCTATCCATGTCGCCCAGATGTTTTCATCCTCCGTGGCTTATCCCT | | |
| | CAGTATTGAGCGAGGAAAGACAGTAGCATTTGTGGGGAGCAGCGGCTGTGGGAAAAGC | | |
| | ACTTCTGTTCAACTTCTGCAGAGACTTTATGACCCCGTGCAAGGACAACAGCTGTTTG | | |
| | ATGGTGTGGATGCAAAAGAATTGAATGTACAGTGGCTCCGTTCCCAAATAGCAATCGT | | |
| | TCCTCAAGAGCCTGTGCTCTTCAACTGCAGCATTGCTGAGAACATCGCCTATGGTGAC | | |
| | AACAGCCGTGTGGTGCCATTAGATGAGATCAAAGAAGCCGCAAATGCAGCAAATATCC | | |
| | ATTCTTTTATTGAAGGTCTCCCTAAATACAACACACAAGTTGGACTGAAAGGAGCACA | | |
| | GCTTTCTGGCGGCCAGAAACAAAGACTAGCTATTGCAAGGGCTCTTCTCCAAAAACCC | | |
| | AAAATTTTATTGTTGGATGAGGCCACTTCAGCCCTCGATAATGACAGTGAGAAGGTAC | | |
| | AGGTGGTTCAGCATGCCCTTGATAAAGCCAGGACGGGAAGGACATGCCTAGTGGTCAC | | |
| | TCACAGGCTCTCTGCAATTCAGAACGCAGATTTGATAGTGGTTCTGCACAATGGAAAG | | |
| | ATAAAGGAACAAGGAACTCATCAAGAGCTCCTGAGAAATCGAGACATATATTTTAAGT | | |
| | TAGTGAATGCACAGTCAGCGAGCAAAGGTCGGACTACAATCGTGGTAGCACACCGACT | | |
| | TTCTACTATTCGAAGTGCAGATTTGATTGTGACCCTAAAGGATGGAATGCTGGCGGAG | | |
| | AAAGGAGCACATGCTGAACTAATGGCAAAACGAGGTCTATATTATTCACTTGTGATGT | | |
| | CACAGGTAATGCTTATGTGACATAATGCTAT | | |
| | ORF Start: ATG at 16 | | ORF Stop: TGA at 4078 |
| | SEQ ID NO:304 | 1354 aa | MW at 149167.3 kD |

| | |
|---|---|
| NOV107a, | MDLEAAKNGTAWRPTSAEGDFELGISSKQKRKKTKTVKMIGVLTLFRYSDWQDKLFMS |
| CG57468-01 Protein | |
| Sequence | LGTIMAIAHGSGLPLMMIVFGEMTDKFVDTAGNFSFPVNFSLSLLNPGKILEEEMTRY |
| | AYYYSGLGAGVLVAAYIQVSFWTLAAGRQIRKIRQKFFHAILRQEIGWFDINDTTELN |
| | TRLTDDISKISEGIGDKVGMFFQAVATFFAGFIVGFIRGWKLTLVIMAISPILGLSAA |
| | VWAKILSAFSDKELAAYAKAGAVAEEALGAIRTVIAFGGQNKELERYQKHLENAKEIG |
| | IKKAISANISMGIAFLLIYASYALAFWYGSTLVISKEYTIGNAMTVFFSILIGAMAIG |
| | ETLVLAPEYSKAKSGAAHLFALLEKKPNIDSRSQEGKKPVSDTCEGNLEFREVSFFYP |
| | CRPDVFILRGLSLSIERGKTVAFVGSSGCGKSTSVQLLQRLYDPVQGQVDGVDAKELN |
| | VQWLRSQIAIVPQEPVLFNCSIAENIAYGDNSRVVPLDEIKEAANAANIHSFIEGLPE |
| | KYNTQVGLKGAQLSGGQKQRLAIARALLQKPKILLLDEATSALDNDSEWQVVQHALDK |
| | ARTGRTCLVVTHRLSAIQNADLIVVLHNGKIKEQGTHQELLRNRDIYFKLVNAQSASK |
| | GRTTIVVAHRLSTIRSADLIVTLKDGMLAEKGAHAELMAKRGLYYSLVMSQVMLMGTL |
| | SDCGNSLPEVSLLKILKLNKPEWPFVVLGTLASVLNGTVHPVFSIIFAKIITVMFGNN |
| | DLLFFLKIFLYSFLLFFLKQGFSVDFCLFAFQGLFYGRAGEILTMRLRHLAFKAMLYQ |
| | DIAWFDEKENSTGGLTTILAIDIAQIQGATGSRIGVLTQNATNMGLSVIISFIYGWEM |
| | TFLILSIAPVLAVTGMIETAAMTGFANKDKQELKHAGKVKIATEALENIRTIVSLTRE |
| | KAFEQMYEEMLQTQHRRNTSKKAQIIGSCYAFSHAFIYFAYAAGFRFGAYLIQAGRMS |
| | NALSFDRVFTAIAYGAMAIGETLVLAPEYSKAKSGAAHLFALLEKKPNIDSRSQEGKK |
| | PLSQDTCEGNLEFREVSFFYPCRPDVFILRGLSLSIERGKTVAFVGSSGCGKSTSVQL |
| | LQRLYDPVQGQQLFDGVDAKELNVQWLRSQIAIVPQEPVLFNCSIAENIAYGDNSRVV |
| | PLDEIKEAANAANIHSFIEGLPKYNTQVGLKGAQLSGGQKQRLAIARALLQKPKILLL |
| | DEATSALDNDSEKVQVVQHALDKARTGRTCLVVTHRLSAIQNADLIVVLHNGKIKEQG |
| | THQELLRNRDIYFKLVNAQSASKGRTTIVVAHRLSTIRSADLIVTLKDGMLAEKGAHA |
| | ELMAKRGLYYSLVMSQVMLM |

Further analysis of the NOV107a protein yielded the following properties shown in Table 107B.

TABLE 107B
Protein Sequence Properties NOV107a

| | |
|---|---|
| PSort | 0.6000 probability located in plasma membrane; 0.4000 |
| analysis: | probability located in Golgi body; 0.3000 probability |
| | located in endoplasmic reticulum (membrane); 0.3000 |
| | probability located in microbody (peroxisome) |
| SignalP | No Known Signal Sequence Predicted |
| analysis. | |

A search of the NOV107a protein against the Geneseq database, a proprietary database that contains sequences published in patents and patent publication, yielded several homologous proteins shown in Table 107C.

TABLE 107C
Geneseq Results for NOV107a

| | | NOV107a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB81064 | Cynomologous monkey P- | 1 . . . 1299 | 750/1312 (57%) | 0.0 |
| | glycoprotein variant 1-Macaca | 1 . . . 1278 | 964/1312 (73%) | |
| | fascicularis, 1280 aa. | | | |
| | [WO200123565-A1, 5 APR. 2001] | | | |
| AAB81065 | Cynomologous monkey P- | 1 . . . 1299 | 749/1312 (57%) | 0.0 |
| | glycoprotein variant 2-Macaca | 1 . . . 1281 | 967/1312 (73%) | |
| | fascicularis, 1283 aa. | | | |
| | [WO200123565-A1, 5 APR. 2001] | | | |
| AAB81959 | Human MDR1-Homo sapiens, | 1 . . . 1299 | 749/1324 (56%) | 0.0 |
| | 1280 aa. [WO200121762-A2, | 1 . . . 1278 | 967/1324 (72%) | |
| | 29 MAR. 2001] | | | |
| AAY58186 | Human wild-type multidrug | 1 . . . 1299 | 749/1324 (56%) | 0.0 |
| | resistance-1 (MDR-1) protein- | 1 . . . 1278 | 967/1324 (72%) | |
| | Homo sapiens, 1280 aa. | | | |
| | [WO9961589-A2, 2 DEC. 1999] | | | |
| AAW44073 | Human multidrug resistance P- | 1 . . . 1299 | 749/1324 (56%) | 0.0 |
| | glycoprotein MDR1-Homo | 1 . . . 1278 | 967/1324 (72%) | |
| | sapiens, 1280 aa. [WO9740160- | | | |
| | A1, 30 OCT. 1997] | | | |

In a BLAST search of public sequence databases, the NOV107a protein was found to have homology to the proteins shown in the BLASTP data in Table 107D.

TABLE 107D
Public BLASTP Results for NOV107a

| | | NOV107a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P23174 | Multidrug resistance protein 3 (P- | 1 . . . 1299 | 818/1303 (62%) | 0.0 |
| | glycoprotein 3)-Cricetulus | 1 . . . 1279 | 999/1303 (75%) | |
| | griseus (Chinese hamster), 1281 | | | |
| | aa. | | | |
| P21440 | Multidrug resistance protein 2 (P- | 1 . . . 1299 | 823/1306 (63%) | 0.0 |
| | glycoprotein 2)-Mus musculus | 1 . . . 1274 | 998/1306 (76%) | |
| | (Mouse), 1276 aa. | | | |
| Q08201 | Multidrug resistance protein 2 (P- | 1 . . . 1299 | 823/1309 (62%) | 0.0 |
| | glycoprotein 2)-Rattus | 1 . . . 1276 | 999/1309 (75%) | |
| | norvegicus (Rat), 1278 aa. | | | |
| CAC37764 | SEQUENCE 1 FROM PATENT | 1 . . . 1299 | 750/1312 (57%) | 0.0 |
| | WO0123565-Macaca fascicularis | 1 . . . 1278 | 964/1312 (73%) | |
| | (Crab eating macaque) | | | |
| | (Cynomolgus monkey), 1280 aa. | | | |
| CAC37765 | SEQUENCE 3 FROM PATENT | 1 . . . 1299 | 749/1312 (57%) | 0.0 |
| | WO0123565-Macaca fascicularis | 1 . . . 1281 | 967/1312 (73%) | |
| | (Crab eating macaque) | | | |
| | (Cynomolgus monkey), 1283 aa. | | | |

PFam analysis predicts that the NOV107a protein contains the domains shown in the Table 107E.

TABLE 107E
Domain Analysis of NOV107a

| | | Identities/ | |
|---|---|---|---|
| Pfam | NOV107a | Similarities for | Expect |
| Domain | Match Region | the Matched Region | Value |
| ABC_membrane: | 57 . . . 350 | 115/301 (38%) | 3.3e−83 |
| domain 1 of 2 | | 252/301 (84%) | |
| MVIN: | 57 . . . 447 | 70/531 (13%) | 5.8 |
| domain 1 of 1 | | 263/531 (50%) | |
| SAA_proteins: | 518 . . . 524 | 6/7 (86%) | 3 |
| domain 1 of 1 | | 7/7 (100%) | |
| ABC_tran: | 424 . . . 609 | 76/199 (38%) | 3.1e−56 |
| domain 1 of 2 | | 150/199 (75%) | |
| DsbD: | 722 . . . 926 | 39/249 (16%) | 9.6 |
| domain 1 of 1 | | 126/249 (51%) | |
| ABC_membrane: | 722 . . . 1008 | 80/297 (27%) | 2.2e−43 |
| domain 1 of 2 | | 222/297 (75%) | |
| ABC_tran: | 1083 . . . 1270 | 77/202 (38%) | 7.1e−54 |
| domain 2 of 2 | | 154/202 (76%) | |
| GidB: | 1170 . . . 1312 | 29/202 (14%) | 6.6 |
| domain 1 of 1 | | 97/202 (48%) | |

### Example 108

The NOV108 clone was analyzed, and the nucleotide and predicted polypeptide sequences are shown in Table 108A.

TABLE 108A
NOV108 Sequence Analysis

| | SEQ ID NO:305 | 520 bp | |
|---|---|---|---|
| NOV 108a, | CCCCGTTCTATCAGCCATGGTCAACCCCACCAGGTTCTTAGACATCATCGTGGATGGT | | |
| CG59609-01 DNA Sequence | | | |
| | GAGCTCTTGGGACGTGTCTCCTTTGAGCTGTTTGCAGACAAGATTCCAAAGACAGCAG | | |
| | AAAATTTTTGTGCTCTAATCATTGGAGAGAAAGGATTTGGTTATAAAGGTTCCTACTT | | |
| | TCACAGAATTGTTCCTGGGTTTATGTGTCAGGGTGGTGACTTCACACAGCATAATGGC | | |
| | ACTGGTGGCAAGTCCATCTACGGGAAGAAATTTGATGATGAGAACTTCGTCCTAAATT | | |
| | ATACAGGTCCTGGCATCTTGTCCGTGGAGAATGCTGGACCCAACACAAATGGTTCCCA | | |
| | GTTTTTCATCTGCACTGCCATGTCTGAGTGGTTGGATGGCATGCAGGTGGTCTTTGGC | | |
| | AAGGGAAGGAAGGTGAGTATTGTGGAAGCCATGGAGTGCTTTGGGTCCACAAATGGCA | | |
| | AGACCAGCAAGAAGATCACCATTGCTGACTGTGGACAACTCTAATAGGTTTGACTT | | |
| | ORF Start: ATG at 17 | | ORF Stop: TAA at 506 |
| | SEQ ID NO:306 | 163 aa | MW at 17734.1 kD |

| | |
|---|---|
| NOV 108a, | MVNPTRFLDIIVDGELLGRVSFELFADKIPKTAENFCALIIGEKGFGYKGSYFHRIVP |
| CG59609-01 Protein Sequence | |
| | GFMCQGGDFTQHNGTGGKSIYGKKFDDENFVLNYTGPGILSVENAGPNTNGSQFFICT |
| | AMSEWLDCMQVVGKCRKVGIVEAMECFGSTNCKTSKKITIADCGQL |

Further analysis of the NOV108a protein yielded the following properties shown in Table 108B.

TABLE 108B
Protein Sequence Properties NOV108a

| | |
|---|---|
| PSort | 0.6400 probability located in microbody (peroxisome); |
| analysis: | 0.6000 probability located in plasma membrane; 0.4500 |
| | probability located in cytoplasm; 0.1000 probability |
| | located in mitochondrial matrix space |
| SignalP | No Known Signal Sequence Predicted |
| analysis: | |

A search of the NOV108a protein against the Geneseq database, a proprietary database that contains sequences published in patents and patent publication, yielded several homologous proteins shown in Table 108C.

TABLE 108C
Geneseq Results for NOV108a

| | | NOV108a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU01195 | Human cyclophilin A protein- | 1 . . . 163 | 134/164 (81%) | 2e−75 |
| | Homo sapiens, 165 aa. | 1 . . . 164 | 147/164 (88%) | |
| | [WO200132876-A2, 10 MAY 2001] | | | |
| AAW56028 | Calcineurin protein-Mammalia, | 1 . . . 163 | 134/164 (81%) | 2e−75 |
| | 165 aa. [WO9808956-A2, | 1 . . . 164 | 147/164 (88%) | |
| | 5 MAR 1998] | | | |
| AAR13726 | Bovine cyclophilin-Bos taurus, | 2 . . . 163 | 133/163 (81%) | 5e−75 |
| | 163 aa. [U.S. Pat. No. 5047512-A, | 1 . . . 163 | 146/163 (88%) | |
| | 10 SEP. 1991] | | | |
| AAG65275 | Haematopoietic stem cell | 2 . . . 163 | 133/163 (81%) | 9e−75 |
| | proliferation agent related human | 1 . . . 163 | 146/163 (88%) | |
| | protein #2-Homo sapiens, 164 aa. | | | |
| | [JP2001163798-A, 19 JUN. 2001] | | | |
| AAP90431 | Cyclophilin-Homo sapiens | 2 . . . 163 | 133/163 (81%) | 9e−75 |
| | (human), 164 aa. [EP326067-A, | 1 . . . 163 | 146/163 (88%) | |
| | 2 AUG. 1989] | | | |

In a BLAST search of public sequence databases, the NOV108a protein was found to have homology to the proteins shown in the BLASTP data in Table 108D.

TABLE 108D
Public BLASTP Results for NOV108a

| | | NOV108a | | |
|---|---|---|---|---|
| Protein | | Residues/ | Identities/ | |
| Accession | | Match | Similarities for | Expect |
| Number | Protein/Organism/Length | Residues | the Matched Portion | Value |
| CAC39529 | SEQUENCE 26 FROM PATENT | 1 . . . 163 | 134/164 (81%) | 8e−75 |
| | WO0132876-Homo sapiens | 1 . . . 164 | 147/164 (88%) | |
| | (Human), 165 aa. | | | |
| Q9BRU4 | PEPTIDYLPROLYL ISOMERASE | 1 . . . 163 | 134/164 (81%) | 2e−74 |
| | A (CYCLOPHILIN A)-Homo | 1 . . . 164 | 146/164 (88%) | |
| | sapiens (Human), 165 aa. | | | |
| P04374 | Peptidyl-prolyl cis-trans isomerase | 2 . . . 163 | 133/163 (81%) | 2e−74 |
| | A (EC 5.2.1.8) (PPIase) (Rotamase) | 1 . . . 163 | 146/163 (88%) | |
| | (Cyclophilin A) (Cyclosporin A- | | | |
| | binding protein)-Bos taurus | | | |
| | (Bovine), and, 163 aa. | | | |
| P05092 | Peptidyl-prolyl cis-trans isomerase | 2 . . . 163 | 133/163 (81%) | 3e−74 |
| | A (EC 5.2.1.8) (PPIase) (Rotamase) | 1 . . . 163 | 146/163 (88%) | |
| | (Cyclophilin A) (Cyclosporin A- | | | |
| | binding protein)-Homo sapiens | | | |
| | (Human),, 164 aa. | | | |
| Q9TTC6 | CYCLOPHILIN 18-Oryctolagus | 1 . . . 163 | 133/164 (81%) | 5e−74 |
| | cuniculus (Rabbit), 164 aa. | 1 . . . 164 | 147/164 (89%) | |

PFam analysis predicts that the NOV108a protein contains the domains shown in the Table 108E.

TABLE 108E
Domain Analysis of NOV108a

| | | Identities/ | |
|---|---|---|---|
| Pfam | NOV108a | Similarities for | Expect |
| Domain | Match Region | the Matched Region | Value |
| pro_isomerase: | 5 . . . 163 | 101/181 (56%) | 5.2e−79 |
| domain 1 of 1 | | 137/181 (76%) | |

### Example 109

The NOV109 clone was analyzed, and the nucleotide and predicted polypeptide sequences are shown in Table 109A.

TABLE 109A
NOV109 Sequence Analysis

| | SEQ ID NO:307 | 887 bp | |
|---|---|---|---|
| NOV109a, | GATATCATTTTTTATGGCAGCCATTGTTAAGCCTCCAGAACCTATACCTTTAAAATGG | | |
| CG59613-01 DNA Sequence | | | |
| | TTAACAGATAAGCCAGTTTGGATAGAACAATGGCCACTGAGTAAAGAGAAACTGGAGG | | |
| | CTTTAGAGGATTTGGTTACTGAACAATTCTCAATAATCATTTTCCAAAAAGTGAACCT | | |
| | ACACAGCATGAAAGTATCACACATTTCCTTAGTGCAGCTAACCCTGTGTGACCAGGGC | | |
| | TTCAACACATACCACTGTGACCACAACCTAGCCATGAGCATGAGCCTCACCAGCATGT | | |
| | CCAAAATGCTAAAATACAACAATGGCAGTGAAGACATCACTACATGGAGGGCTGAAGG | | |
| | TACTATGGATCTCTTGGTGCTAGAATTTGAAGCACTAAATCAAGAGAACTTTGTGGAC | | |
| | TGTGAATTGAAGTTAATGACTCTAGATGTTGAGCAACTTGAAATTCCAGAACAAGAGT | | |
| | ACAGCTGTGTAATAAAGATGCATTCTAGTGAATTTGTTCATATATGCCAAGATCTCAG | | |
| | TCATATTGGAGAGTCTGCTATAATTTCTTGTGCAAAAGATGGAGTGAATTTTTCTGCA | | |
| | AATGGAGAACTTGGACATGGAAACATTGCCACAATTGCCCAAACAAGTAATTACAATA | | |
| | AAGAAGAGGAGGCTGTTGCCATAATGATGAATGGGCCAGTTCAGCTAACTTTTGCACT | | |
| | AAGTTACTTAAATTTCTTTATAACAGGCACTCCACTCTCTCAGATGCACCCCTTGCTG | | |
| | GAGAGTATAAGATTGCCGGATATGGAACATTTAAAGTATTATTTGGCTCCCAAAATTG | | |
| | AGGATGAAAAAGGATTTTAGAAATTCTTAGAATCCAAGAAAATAAAACTAAGCTCTTT | | |
| | GAAAATTGCTTCTGAGA | | |
| | ORF Start: ATG at 14 | | ORF Stop: TAG at 830 |
| | SEQ ID NO:308 | 272 aa | MW at 30831.1 kD |

| | |
|---|---|
| NOV109a, | MAAIVKPPEPIPLKWLTDKPVWIEQWPLSKEKLEALEDLVTEQFSIIIFQKVNLHSMK |
| CG59613-01 Protein Sequence | VSHISLVQLTLCDQGFNTYHCDHNLAMSMSLTSMSKMLKYNNGSEDITTWRAEGTMDL |
| | LVLEFEALNQENFVDCELKLMTLDVEQLEIPEQEYSCVIKMHSSEFVHICQDLSHIGE |
| | SAIISCAKDGVNFSANGELGHGNIATIAQTSNYNKEEEAVAIMMNGPVQLTFALSYLN |
| | FFITGTPLSQMHIPLLESIRLPDMEHLKYYLAPKIEDEKGF |

Further analysis of the NOV109a protein yielded the following properties shown in Table 109B.

TABLE 109B
Protein Sequence Properties NOV109a

| | |
|---|---|
| PSort | 0.6500 probability located in cytoplasm; 0.1000 probability |
| analysis: | located in mitochondrial matrix space; 0.1000 probability |
| | located in lysosome (lumen); 0.0000 probability located in |
| | endoplasmic reticulum (membrane) |
| SignalP | Likely cleavage site between residues 19 and 20 |
| analysis: | |

A search of the NOV109a protein against the Geneseq database, a proprietary database that contains sequences published in patents and patent publication, yielded several homologous proteins shown in Table 109C.

TABLE 109C
Geneseq Results for NOV109a

| | | NOV109a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAY51639 | Human PCNA protein fragment- | 25 . . . 271 | 158/255 (61%) | 8e−78 |
| | Homo sapiens, 261 aa. | 8 . . . 260 | 184/255 (71%) | |
| | [WO200008164-A2, 17 FEB. 2000] | | | |
| AAY52010 | Human PCNA protein-Homo | 25 . . . 271 | 158/255 (61%) | 8e−78 |
| | sapiens, 261 aa. [DE19840771-A1, | 8 . . . 260 | 184/255 (71%) | |
| | 10 FEB. 2000] | | | |
| AAB43712 | Human cancer associated protein | 25 . . . 271 | 158/255 (61%) | 8e−78 |
| | sequence SEQ ID NO: 1157-Homo | 16 . . . 268 | 184/255 (71%) | |
| | sapiens, 269 aa. [WO200055350- | | | |
| | A1, 21 SEP. 2000] | | | |
| AAG75139 | Human colon cancer antigen | 25 . . . 269 | 157/253 (62%) | 5e−77 |
| | protein SEQ ID NO: 5903-Homo | 16 . . . 266 | 182/253 (71%) | |
| | sapiens, 268 aa. [WO200122920- | | | |
| | A2, 5 APR. 2001] | | | |
| AAW90758 | Human PCNA protein fragment #2- | 39 . . . 268 | 149/238 (62%) | 7e−73 |
| | Homo sapiens, 236 aa. | 1 . . . 236 | 171/238 (71%) | |
| | [DE19840771-A1, 10 FEB. 2000] | | | |

In a BLAST search of public sequence databases, the NOV109a protein was found to have homology to the proteins shown in the BLASTP data in Table 109D.

TABLE 109D
Public BLASTP Results for NOV109a

| | | NOV109a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P12004 | Proliferating cell nuclear antigen | 25 . . . 271 | 158/255 (61%) | 3e−77 |
| | (PCNA) (Cyclin)-Homo sapiens | 8 . . . 260 | 184/255 (71%) | |
| | (Human), 261 aa. | | | |
| P04961 | Proliferating cell nuclear antigen | 25 . . . 271 | 158/255 (61%) | 5e−77 |
| | (PCNA) (Cyclin)-Rattus norvegicus | 8 . . . 260 | 185/255 (71%) | |
| | (Rat), 261 aa. | | | |
| P57761 | Proliferating cell nuclear antigen | 25 . . . 271 | 158/255 (61%) | 7e−77 |
| | (PCNA)-Cricetulus griseus | 8 . . . 260 | 184/255 (71%) | |
| | (Chinese hamster), 261 aa. | | | |
| Q91ZH2 | 11 DAYS EMBRYO CDNA, | 25 . . . 272 | 156/256 (60%) | 1e−75 |
| | RIKEN FULL-LENGTH | 8 . . . 261 | 183/256 (70%) | |
| | ENRICHED LIBRARY, | | | |
| | CLONE: 2700095L20, FULL | | | |
| | INSERT SEQUENCE-Mus | | | |
| | musculus (Mouse), 261 aa. | | | |
| P17918 | Proliferating cell nuclear antigen | 25 . . . 270 | 155/254 (61%) | 5e−75 |
| | (PCNA) (Cyclin)-Mus musculus | 8 . . . 259 | 182/254 (71%) | |
| | (Mouse), 261 aa. | | | |

PFam analysis predicts that the NOV109a protein contains the domains shown in the Table 109E.

TABLE 109E
Domain Analysis of NOV109a

| | | Identities/ | |
|---|---|---|---|
| Pfam | NOV109a | Similarities for | Expect |
| Domain | Match Region | the Matched Region | Value |
| PCNA: | 23 . . . 143 | 46/128 (36%) | 2.3e−20 |
| domain 1 of 1 | | 83/128 (65%) | |
| PCNA_C: | 145 . . . 265 | 59/131 (45%) | 1.6e−45 |
| domain 1 of 1 | | 98/131 (75%) | |

### Example 110

The NOV110 clone was analyzed, and the nucleotide and predicted polypeptide sequences are shown in Table 110A.

TABLE 110A
NOV110 Sequence Analysis

| | SEQ ID NO:309 | 1233 bp | |
|---|---|---|---|
| NOV110a, | TGGCAATGGAAGAAGAGATCCCCGCGCTCTTCATTGACAATGGCTCCGGCATGTGGAA | | |
| CG59619-01 DNA Sequence | | | |
| | AGCAGCTTTGCTGGGAGACAATGCCCTCCGAGCCATATTCCCCTCCATCATCGGGCAC | | |
| | CCCCGGCACCAGGGCGTGATGGTGGGCATGGGCCAGAAGGACTCCTACGTGGGCGACC | | |
| | AGGCCCAGAGCAAGTGCGGCATCCTGACCCTGAAGTACCCCATCAAGCATGGCATCGT | | |
| | CACAAACTGGGACGACATGGAGAAGATCTGGCACCATGTTTTCTACAACGAGCTGTGC | | |
| | GTGGCCCTGGAGGAGCAGGTGGTGCTGCTGACCGAGGCCCCGCTAAACCCCAGGGCCA | | |
| | ATAGGGAGAAGATGACTCAGATCATGTTTAAGACCTTCAACACCCAGGCCATGTACGT | | |
| | GGCCATTCAGGCCGTGCTGACCCTCCACAGCTCTGGTTGCACCACTGGCATTGTCATG | | |
| | GACTCTGGAGATGGGGTCACCCACACAGTGCCCATCTACGAGCGCCACACCCTCCCTC | | |
| | ACACCATCTTGCATCTGGACCTGGCTGGCCAGGACCTTACTGACTACCTCATGAAGAT | | |
| | CCCTACCTACCGCAGCTATAGCTTCAACACCATGGCCAAGTGGAAAATCGTGCGCAAC | | |
| | ATCAAGGAGAAGCTATGCTATGTCGCTCTGGACTTCGAGGAGGAGATGGCCACTGCTG | | |
| | CATCCTCCTCCTCCCTGGAGAAGAGCTACGAGCTGCCTGACAGCCAGGCCATCATTAT | | |
| | TAGCAATGAGCGGTTCCGGTGTCCGGAGGCACTGTTCCAGCCTTCCTTCCTGGGCATG | | |
| | GAATCCTGTGGCATCCATGAAAGTACCTTCAACTCCATCATGAAGTGTGATATGGACA | | |
| | TCCCCAAAGACCTGTACGCCAACACAGTGCTGTCTGGCGTCACCACCATGTACCCTGG | | |
| | CATCCCCAATAGGATGCAGAAGGAGATCACTGCCCTGGCATCCAGCACCATGAAGATC | | |
| | AAGATATCGTGCCCCATCGTGCCCCCAGAGTGCAAGTACTTTGTGTGGATCGGTGGCT | | |
| | CCATCCTGGCCTCACTGTCCACCTTCCAGCAGATGTGGATTAGCAAGCAGGAGTACAA | | |
| | CGAGTCGGGCCCCTCCATCATCCACCGCAAATGGACTGCGAGCAGATGCATAGCATTT | | |
| | GCTGCATGGGTTAATTCAGAAGTATAAATTTGCCCCTGGCAAATGCATATACCTCATG | | |
| | CTAGCCTCACGATAC | | |
| | ORF Start: ATG at 6 | | ORF Stop: TAA at 1185 |
| | SEQ ID NO:310 | 393 aa | MW at 44147.5 kD |

| | |
|---|---|
| NOV110A, | MEEEIPALFIDNGSGMWKAALLGDNALRAIFPSIIGHPRHQGVMVGMGQKDSYVGDQA |
| CG59619-01 Protein Sequence | |
| | QSKCGILTLKYPIKHGIVTNWDDMEKIWHHVFYNELCVALEEQVVLLTEAPLNPRANR |
| | EKMTQIMFKTFNTQAMYVAIQAVLTLHSSGCTTGIVMDSGDGVTHTVPIYERHTLPHT |
| | ILHLDLAGQDLTDYLMKIPTYRSYSFNTMAKWKIVRNIKEKLCYVALDFEEEMATAAS |
| | SSSLEKSYELPDSQAIIISNERFRCPEALFQPSFLGMESCGIHESTFNSIMKCDMDIP |
| | KDLYANTVLSGVTTMYPGIPNRMQKEITALASSTMKIKISCPIVPPECKYFVWIGGSI |
| | LASLSTFQQMWISKQEYNESGPSIIHRKWTASRCTAFAAWVNSEV |

Further analysis of the NOV110a protein yielded the following properties shown in Table 110B.

TABLE 110B
Protein Sequence Properties NOV110a

| | |
|---|---|
| PSort | 0.4500 probability located in cytoplasm; 0.1547 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV110a protein against the Geneseq database, a proprietary database that contains sequences published in patents and patent publication, yielded several homologous proteins shown in Table 110C.

TABLE 110C
Geneseq Results for NOV110a

| | | | | |
|---|---|---|---|---|
| | | NOV110A | Identities/ | |
| | | Residues/ | Similarities for | |
| Geneseq | Protein/Organism/Length [Patent | Match | the Matched | Expect |
| Identifier | +190, Date] | Residues | Region | Value |

| | | | | |
|---|---|---|---|---|
| AAU32060 | Novel human secreted protein | 1..3764 315/376 (83%) | e−180 | |
| | #2551 - Homo sapiens, 399 aa. | 25..397 | 336/376 (88%) | |
| | [WO200179449-A2, 25 OCT. 2001] | | | |
| AAB43991 | Human cancer associated protein | 1..376 | 311/376 (82%) | e−179 |
| | sequence SEQ ID NO:1436 - Homo. | 39..411 | 336/376 (88%) | |
| | sapiens, 413 aa. [WO200055350- | | | |
| | A1, 21 SEP. 2000] | | | |
| AAP61532 | Sequence of beta-actin - Homo | 1..376 | 311/376 (82%) | e−179 |
| | sapiens, 375 aa. [EP174608-A, 19 | 1..373 | 335/376 (88%) | |
| | MAR. 1986] | | | |
| AAB12985 | Human beta-actin protein sequence | 2..376 | 310/375 (82%) | e−178 |
| | - Homo sapiens, 374 aa. | 1..372 | 334/375 (88%) | |
| | [US6087398-A, 11 JUL. 2000] | | | |
| AAR50328 | Drug resistant structural protein - | 1..376 | 309/376 (82%) | e−178 |
| | Homo sapiens, 375 aa. | 1..373 | 335/376 (88%) | |
| | [JP06038773-A, 15 FEB. 1994] | | | |

In a BLAST search of public sequence databases, the NOV110a protein was found to have homology to the proteins shown in the BLASTP data in Table 110D.

TABLE 110D
Public BLASTP Results for NOV110a

| | | NOV110a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P02571 | Actin, cytoplasmic 2 (Gamma-actin)- | 1 . . . 376 | 315/376 (83%) | e−179 |
| | Homo sapiens (Human),, 375 aa. | 1 . . . 373 | 336/376 (88%) | |
| ATBOG | actin gamma (tentative sequence)- | 2 . . . 376 | 314/375 (83%) | e−179 |
| | bovine, 374 aa. | 1 . . . 372 | 335/375 (88%) | |
| P53505 | Actin, cytoplasmic type 5-Xenopus | 2 . . . 376 | 313/375 (83%) | e−178 |
| | laevis (African clawed frog), 376 aa. | 3 . . . 374 | 335/375 (88%) | |
| P29751 | Actin, cytoplasmic 1 (Beta-actin)- | 1 . . . 376 | 311/376 (82%) | e−178 |
| | Oryctolagus cuniculus (Rabbit), 375 | 1 . . . 373 | 337/376 (88%) | |
| | aa. | | | |
| O93400 | ACTIN, CYTOPLASMIC 1 | 1 . . . 376 | 311/376 (82%) | e−178 |
| | (BETA-ACTIN) (CYTOPLASMIC | 1 . . . 373 | 336/376 (88%) | |
| | BETA ACTIN)-Xenopus laevis | | | |
| | (African clawed frog), 375 aa. | | | |

PFam analysis predicts that the NOV110a protein contains the domains shown in the Table 110E.

TABLE 110E
Domain Analysis of NOV110a

| | | Identities/ | |
|---|---|---|---|
| | | Similarities | |
| | NOV110a | for the | Expect |
| Pfam Domain | Match Region | Matched Region | Value |
| actin: domain 1 of 1 | 1 . . . 378 | 284/382 (74%) | 2.2e−227 |
| | | 336/382 (88%) | |

### Example 111.

The NOV111 clone was analyzed, and the nucleotide and predicted polypeptide sequences are shown in Table 111A.

TABLE 111A
NOV111 Sequence Analysis

| | SEQ ID NO:311 | 1197 bp | |
|---|---|---|---|
| NOV111a, | AACCATGTCTAAGCGGGAGTCCTTTAACCTGGAAAGTTATGAATTGGACAAAAGCTTC | | |
| CG59621-01 DNA Sequence | | | |
| | TGGCTAACCAGATTCACTGAACTGAAGGGCACAGGTTGCAAAGTGCCCCAAGATGTCT | | |
| | TGCAAAAATTGCTGGAATCTTTACAGGAGAACCACTTCCAAGAAGATGAGCAGTTTCT | | |
| | GGGAGCCGTTATGCCAAGGCTTCGCATTGGAATGGATACTTGTGCCATTTCTTTGAGG | | |
| | CATGGTGGGCTTTCCTTGGTTCAAACCACAGATTACATTTACCCGATCGTAGACGACC | | |
| | CTTACATGATGGGCAGGATAGCATGTGCCAATGTCCTCAGTGACCTCTATGCAATGGG | | |
| | GGTCACAGAATGTGACAATATGCTGATGCTCCTTGGAGTCAGTAATAAAATGACCGAC | | |
| | AGGGAAAGGGATAAAGTGATGCCTCTGATTATCCAAAGTTTTAAAGATGCAGCTGAGG | | |
| | AAGCAGGAATGTCTGTAATGGTCAGCCAAACAGTACTAAATCCCTGGATTGTCCTGGG | | |
| | AGGAGTCACTACCACTGTCTTCCAGCCCAATGAATTTATCATGCCAGACAATGCAGTG | | |
| | CCAGGGGACGTGCTGGTGTTGACAAAACCCCTGGGGACACAGGTGGCAGTGGCTGTGC | | |
| | ACCAGTGGCTGGATATTCCTTTGAAATGGAATAAGATTAAGCTAGTGGTCACCGAAGA | | |
| | TGTAGAGCTGGCCAACCAGGAGGCGATGATGAACATGGTGAGGCTCAACAGGACAGCT | | |
| | GCAGGACTCATGCACACGTTCAATGCCCACATGGCCACTGACATCACGGGCTTCGGGA | | |
| | TTTTGGGCCACGTGCAGAACCTAGCCAAGCAGCAGAGGAACGAGGTGTCGTTTGTAAT | | |
| | TCACAACCTCCTGGTGCTGGCCAAGATGGCTGCGGTGAGCAAGGCCTGCGGAAACATG | | |
| | TTCAGCCTCATGCATGGGACCTGCCCGGAGACCTCAGGCGGCCTTCTGATCTGTTTAC | | |
| | CATGTCAGCAAGCAGCTCGGTTCTGTGCAGAGATAAAGTCCCCCAAATATAGTGAAGG | | |
| | CCACCAAGCATGGATTATTGGGATTGTAGAGAAGGGCAACCACACAGCCAGAATCATA | | |
| | GACAAACCCCAGATCATCAAGGTTGCACCACAAGTGGCCACTCAAAATGTGAATCTCA | | |
| | CACCCGGGGCCACATCTTAATCTAGACAGAAATAGCT | | |
| | ORF Start: ATG at 5 | | ORF Stop: TAA at 1178 |
| | SEQ ID NO:312 | 391 aa | MW at 43193.9 kD |

| | |
|---|---|
| NOV111a, | MSKRESFNLESYELDKSFWLTRFTELKGTGCKVPQDVLQKLLESLQENHFQEDEQFLG |
| CG59621-01 Protein Sequence | |
| | AVMPRLRIGMDTCAISLRHGGLSLVQTTDYIYPIVDDPYMMGRIACANVLSDLYAMGV |
| | TECDNMLMLLGVSNKMTDRERDKVMPLIIQSFKDAAEEAGMSVMVSQTVLNPWIVLGG |
| | VTTTVFQPNEFIMPDNAVPGDVLVLTKPLGTQVAVAVHQWLDIPLKWNKIKLVVTEDV |
| | ELANQEAMMNMVRLNRTAAGLMHTFNAHMATDITGFGILGHVQNLAKQQRNEVSFVIH |
| | NLLVLAKMAAVSKACGNMFSLMHGTCPETSGGLLICLPCQQAARFCAEIKSPKYSEGH |
| | QAWIIGIVEKGNHTARIIDKPQIIKVAPQVATQNVNLTPGATS |

Further analysis of the NOV111a protein yielded the following properties shown in Table 111B.

TABLE 111B
Protein Sequence Properties NOV111a

| | |
|---|---|
| PSort | 0.8500 probability located in endoplasmic reticulum |
| analysis: | (membrane); 0.4400 probability located in plasma membrane; |
| | 0.1000 probability located in mitochondrial inner membrane; |
| | 0.1000 probability located in Golgi body |
| SignalP | No Known Signal Sequence Predicted |
| analysis: | |

A search of the NOV111a protein against the Geneseq database, a proprietary database that contains sequences published in patents and patent publication, yielded several homologous proteins shown in Table 111C.

TABLE 111C
Geneseq Results for NOV111a

| | | NOV111a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAB58174 | Lung cancer associated polypeptide | 166 . . . 391 | 168/227 | (74%) | 2e−88 |
| | sequence SEQ ID 512 - Homo | 20 . . . 243 | 189/227 | (83%) | |
| | sapiens, 250 aa. [WO200055180- | | | | |
| | A2, 21 SEP 2000] | | | | |
| AAO01161 | Human polypeptide SEQ ID NO | 147 . . . 264 | 81/119 | (68%) | 2e−36 |
| | 15053 - Homo sapiens, 122 aa. | 1 . . . 118 | 92/119 | (77%) | |
| | [WO200164835-A2, 7 SEP 2001] | | | | |
| AAB53700 | Human colon cancer antigen protein | 42 . . . 99 | 53/58 | (91%) | 1e−24 |
| | sequence SEQ ID NO:1240 - Homo | 1 . . . 58 | 54/58 | (92%) | |
| | sapiens, 106 aa. [WO200055351- | | | | |
| | A1, 21 SEP 2000] | | | | |

In a BLAST search of public sequence databases, the NOV111a protein was found to have homology to the proteins shown in the BLASTP data in Table 111D.

TABLE 111D
Public BLASTP Results for NOV111a

| | | NOV111a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BVT4 | SELENOPHOSPHATE | 1 . . . 391 | 364/392 (92%) | 0.0 |
| | SYNTHETASE, HUMAN | 1 . . . 392 | 367/392 (92%) | |
| | SELENIUM DONOR PROTEIN - | | | |
| | Homo sapiens (Human), 392 aa. | | | |
| P49903 | Selenide,water dikinase 1 (EC | 1 . . . 375 | 348/376 (92%) | 0.0 |
| | 2.7.9.3) (Selenophosphate | 1 . . . 376 | 351/376 (92%) | |
| | synthetase 1) (Selenium donor | | | |
| | protein 1) - Homo sapiens | | | |
| | (Human), 383 aa. | | | |
| AAC50958 | SELENOPHOSPHATE | 2 . . . 391 | 272/411 (66%) | e−147 |
| | SYNTHETASE 2- Homo sapiens | 33 . . . 441 | 313/411 (75%) | |
| | (Human), 448 aa. | | | |
| Q99611 | Selenide,water dikinase 2 (EC | 2 . . . 391 | 272/411 (66%) | e−147 |
| | 2.7.9.3) (Selenophosphate | 33 . . . 441 | 313/411 (75%) | |
| | synthetase 2) (Selenium donor | | | |
| | protein 2) - Homo sapiens | | | |
| | (Human), 448 aa. | | | |
| AAC53024 | SELENOPHOSPHATE | 2 . . . 387 | 267/407 (65%) | e−146 |
| | SYNTHETASE 2 - Mus musculus | 36 . . . 441 | 307/407 (74%) | |
| | (Mouse), 452 aa. | | | |

PFam analysis predicts that the NOV111a protein contains the domains shown in the Table 111E.

TABLE 111E
Domain Analysis of NOV111a

| | | Identities/ | | |
|---|---|---|---|---|
| | | Similarities | | |
| | NOV111a | for the | | Expect |
| Pfam Domain | Match Region | Matched Region | | Value |
| AIRS: domain 1 of 1 | 32 . . . 188 | 29/180 | (16%) | 3e−18 |
| | | 113/180 | (63%) | |
| AIRS_C: | 191 . . . 367 | 34/197 | (17%) | 1.1e−20 |
| domain 1 of 1 | | 125/197 | (63%) | |

### Example 112.

The NOV112 clone was analyzed, and the nucleotide and predicted polypeptide sequences are shown in Table 112A.

TABLE 112A
NOV112 Sequence Analysis

| | SEQ ID NO:313 | 544 bp | |
|---|---|---|---|
| NOV112a, | CGATGGGACACAGACAGGTCACCCCAGCTCTGATCTTTGCCATCACAGTTGCTACAAT | | |
| CG59625-01 DNA Sequence | | | |
| | CGGCTCTTTCCAGTTTGGCTACAACACTGGGGTCATCAATGCTCCTGAGACGGTGCAG | | |
| | ATCATAAAGGAATTTATCAATAAAACTTTGACGGACAAGGCAAATGCCCCTCCCTCTG | | |
| | AGGTGCTGCTCACGAATCTCTGGTCCTTGTCTGTGGCCATATTTTCCGTCGGGGGTAT | | |
| | GATCGGCTCCTTTTCCGTCGGACTCTTTGTTAACCGCTTTGGCAGGAGGCGCAATTCA | | |
| | ATGCTGATTGTCAACCTGTTGGCTGCCACTGGTGGCTGCCTTATGGGACTGTGTAAAA | | |
| | TAGCTGAGTCAGTTGAAATGCTGATCCTGGGCCGCTTGGTTATTGGCCTCTTCTGCGG | | |
| | ACTCTGCACAGGTTTTGTGCCCATGTACATTGGAGAGATCTCGCCTACTGCCCTGAGG | | |
| | GGTGCCTTTGGCACTCTCAACCAGCTGGGCATAGTTATTGGAATTCTGGTGGCCCAGG | | |
| | TAATCTTTGGTCTGGAACTCATCCTTGGGTCTGAAGAGCTATGGCCGGTGCTATTAGG | | |
| | CTTTACCATCCTTCCAGCTATCCTGCAAAGTGCAGCCCTTCCATGTTGCCCTGAAAGT | | |
| | CCCAGATTTTTGCTCATTAACAGAAAAAAAGAGGAGAATGCTACGCGGGTCCTCCAGC | | |
| | GGTTGTGGGGCACCCAGGATGTATCCCAAGACATCCAGGAGATGAAAGATGAGAGTGC | | |
| | AAGGATGTCACAAGAAAAGCAAGTCACCGTGCTGGAGCTCTTTAGAGTGTCCAGCTAC | | |
| | CGACAGCCCATCATCATTTCCATTGTGCTCCAGCTCTCTCAGCAGCTCTCTGGGATCA | | |
| | ATGCTGTGGTGTTCTATTACTCAACAGGAATCTTCAAGGATGCAGGTGTTCAACAGCC | | |
| | CATCTATGCCACCATCAGCGCGGGTGTGGTTAATACTATCTTCACTTTACTTTCTGTA | | |
| | GTAGCTCAGATGCTGTTTTCATGGAAAGGAAAACTGAAGTTTCATGTCATAACTGTTT | | |
| | CTTTGTTATTAAAGCTGGGTTACACTGTCTTTAAATTTAATCTTCTGTGTTCCTTCCT | | |
| | CTTACAGAATCACTATAATGGGATGAGCTTTGTCTGTATTGGGGCTATCTTGGTCTTT | | |
| | GTGGCCTGTTTTGAAATTGGACCAGGCCCCATTCCCTGGTTTATTGTGGCCGAACTCT | | |
| | TCAGCCAGGGCCCCCGCCCAGCTGCGATGGCAGTGGCCGGCTGCTCCAACTGGACCTC | | |
| | CAACTTCCTAGTCGGATTGCTCTTCCCCTCTGCTGCTTACTATTTAGGAGCCTACGTT | | |
| | TTTATTATCTTCACCGGCTTCCTCATTACCTTCTTGGCCTTTACCTTCTTCAAAGTCC | | |
| | CTGAGACCCGTGGCAGGACTTTTGAGGATATCACACGGGCCTTTGAAGGGCAGGCACA | | |
| | CGGTGCAGATAGATCTGGGAAGGACGGCGTCATGGGGATGAACAGCATCGAGCCTGCT | | |
| | AAGGAGACCACCACCAATGTCTAAGTCATGCCTCCT | | |
| | ORF Start: ATG at 3 | | ORF Stop: TAA at 1530 |
| | SEQ ID NO:314 | 509 aa | MW at 55571.7 kD |

| | |
|---|---|
| NOV112a, | MGHRQVTPALIFAITVATIGSFQFGYNTGVINAPETVQIIKEFINKTLTDKANAPPSE |
| CG59625-01 Protein Sequence | |
| | VLLTNLWSLSVAIFSVGGMIGSFSVGLFVNRFGRRRNSMLIVNLLAATGGCLMGLCKI |
| | AESVEMLILGRLVIGLFCGLCTGFVPMYIGEISPTALRGAFGTLNQLGIVIGILVAQV |
| | IFGLELILGSEELWPVLLGFTILPAILQSAALPCCPESPRFLLINRKKEENATRVLQR |
| | LWGTQDVSQDIQEMKDESARMSQEKQVTVLELFRVSSYRQPIIISIVLQLSQQLSGIN |
| | AVVFYYSTGIFKDAGVQQPIYATISAGVVNTIFTLLSVVAQMLFSWKGKLKFHVITVS |
| | LLLKLGYTVFKFNLLCSFLLQNHYNGMSFVCIGAILVFVACFEIGPGPIPWFIVAELF |
| | SQGPRPAAMAVAGCSNWTSNFLVGLLFPSAAYYLGAYVFIIFTGFLITFLAFTFFKVP |
| | ETRGRTFEDITRAFEGQAHGADRSGKDGVMGMNSIEPAKETTTNV |

Further analysis of the NOV112a protein yielded the following properties shown in Table 112B.

TABLE 112B
Protein Sequence Properties NOV112a

| | |
|---|---|
| Psort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic reticulum (lumen) |
| SignalP | Likely cleavage site between residues 22 and 23 |
| analysis: | |

A search of the NOV112a protein against the Geneseq database, a proprietary database that contains sequences published in patents and patent publication, yielded several homologous proteins shown in Table 112C.

TABLE 112C
Geneseq Results for NOV112a

| | | NOV112a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY27289 | Glucose transporter protein | 1 . . . 505 | 389/505 (77%) | 0.0 |
| | GLUT3 - Homo sapiens, 494 aa. | 1 . . . 492 | 431/505 (85%) | |
| | [US5942398-A, 24 AUG 1999] | | | |
| AAR11360 | Glucose Transporter Protein from | 4 . . . 491 | 289/489 (59%) | e−156 |
| | CHO cells - Cricetulus sp, 492 aa. | 6 . . . 481 | 364/489 (74%) | |
| | [WO9103554-A, 21 MAR 1991] | | | |
| AAW17835 | Human glucose transporter GLUT- | 4 . . . 491 | 287/489 (58%) | e−155 |
| | 1 - Homo sapiens, 492 aa. | 6 . . . 481 | 362/489 (73%) | |
| | [WO9715668-A2, 1 MAY 1997] | | | |
| AAW93000 | Human GLUT1 protein - Homo | 4 . . . 491 | 284/489 (58%) | e−153 |
| | sapiens, 492 aa. [WO9618957-A1, | 6 . . . 481 | 360/489 (73%) | |
| | 20 JUN 1996] | | | |
| AAB30522 | Amino acid sequence of a | 6 . . . 501 | 289/496 (58%) | e−151 |
| | consensus GLUT polypeptide - | 10 . . . 490 | 357/496 (71%) | |
| | Synthetic, 493 aa. [US6136547-A, | | | |
| | 24 OCT 2000] | | | |

In a BLAST search of public sequence databases, the NOV112a protein was found to have homology to the proteins shown in the BLASTP data in Table 112D.

TABLE 112D
Public BLASTP Results for NOV112a

| | | NOV112a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P11169 | Solute carrier family 2, facilitated | 1 . . . 509 | 446/510 (87%) | 0.0 |
| | glucose transporter, member 3 | 1 . . . 496 | 468/510 (91%) | |
| | (Glucose transporter type 3, brain) - | | | |
| | Homo sapiens (Human), 496 aa. | | | |
| P47842 | Solute carrier family 2, facilitated | 1 . . . 507 | 400/507 (78%) | 0.0 |
| | glucose transporter, member 3 | 1 . . . 494 | 446/507 (87%) | |
| | (Glucose transporter type 3, brain) - | | | |
| | Canis familiaris (Dog), 495 aa. | | | |
| P47843 | Solute carrier family 2, facilitated | 1 . . . 505 | 389/505 (77%) | 0.0 |
| | glucose transporter, member 3 | 1 . . . 492 | 431/505 (85%) | |
| | (Glucose transporter type 3, brain) - | | | |
| | Ovis aries (Sheep), 494 aa. | | | |
| P58352 | Solute carrier family 2, facilitated | 1 . . . 505 | 390/505 (77%) | 0.0 |
| | glucose transporter, member 3 | 1 . . . 492 | 431/505 (85%) | |
| | (Glucose transporter type 3, brain) - | | | |
| | Bos taurus (Bovine), 494 aa. | | | |
| Q07647 | Solute carrier family 2, facilitated | 1 . . . 508 | 380/508 (74%) | 0.0 |
| | glucose transporter, member 3 | 1 . . . 492 | 422/508 (82%) | |
| | (Glucose transporter type 3, brain) - | | | |
| | Rattus norvegicus (Rat), 493 aa. | | | |

PFam analysis predicts that the NOV112a protein contains the domains shown in the Table 112E.

TABLE 112E
Domain Analysis of NOV112a

| | | Identities/ | | |
|---|---|---|---|---|
| | | Similarities | | |
| | NOV112a | for the | | Expect |
| Pfam Domain | Match Region | Matched Region | | Value |
| Herpes_glycop: | 1 . . . 249 | 40/417 | (10%) | 7.2 |
| domain 1 of 1 | | 171/417 | (41%) | |
| GntP_permease: | 65 . . . 329 | 70/478 | (15%) | 2.5 |
| domain 1 of 1 | | 185/478 | (39%) | |
| sugar_tr: | 12 . . . 478 | 188/503 | (37%) | 2.2e−158 |
| domain 1 of 1 | | 410/503 | (82%) | |

### Example 113

The NOV113 clone was analyzed, and the nucleotide and predicted polypeptide sequences are shown in Table 113A.

TABLE 113A
NOV113 Sequence Analysis

| | SEQ ID NO:315 | 1731 bp | |
|---|---|---|---|
| NOV113a, | ACTACTTCGCCGACACTCGCCAGCCTCGGCTACGAGCAAAAAATGCACCGCACCATGA | | |
| CG59887-01 DNA Sequence | | | |
| | GCTCGTTCACCTCGTTTGCCCTGGCCTTTTCCATGGTCTCGATCAACACCGGCGTGGT | | |
| | CACGCTGTTCGCCGACCCGTTCAACCGCGTCGGGGGCATCGGCATCCTCCTGTGGCTG | | |
| | TTGGTGATCCCGCTGGTGTGCTGCATCGTCATGGTCTACTGCCACCTGGCCGGGCGCA | | |
| | TTCCGCTCACCGGCTACGCCTACCAATGGTCCAGCCGATTGGCGGGCAATCACTTCGG | | |
| | CTGGTTTACCGGCTGGGTGGCGTTCACCTCGTTTGTCGCCGGTACAGCCGCCACCTCG | | |
| | GCGGCCATCGGTACGGTGTTCGCACCGGAGATCTGGGCCAACCCGACACAGGGTCAGA | | |
| | TCCAGGGCCTGAGCATCGGCGCCACGCTGGTGGTGGGCTTGCTGAATATCTGCGGGAT | | |
| | TCGCCTGGCCACCCGGATCAACGACATCGGCGCGATCATCGAAATCATCGGCACGGTA | | |
| | CTGCTGGCGATTGCGTTGTTCTTCGGGGTGTTTTTCTTCTTTGAGCACACCCAGGGCG | | |
| | TGGCGATCCTGACCTCCGCGCAACCAGTGAGCGGCGGCACGCTCAGCTTCACCACCAT | | |
| | CGCCCTCGCCACCTTGCTGCCGGTCTCGGTGCTGCTGGGTTGGGAAGGCGCCGCCGAC | | |
| | CTGTCCGAGGAAACCAAGGACCCACGCCGCGCCGCGCCCCGGGCGATGATTCGTGCGG | | |
| | TGCTGGTGTCCAGCGTATTGGGCTTCGTGGTGTTCGCCTTGCTGAGCATCGCGATCCC | | |
| | GGGCTCGGTCAGCGAACTGCTCAGCCACAGCGAAAACCCGGTGATCAATATCGTGCGC | | |
| | CTGCAACTGGGCAATGCCGCCGGCGTGGGCATGATCGTGATCGCTTTCGCCTCGATCC | | |
| | TCGCCTGCCTGATCGCCAACATGGCGGTGGCCACGCGCATGACCTTCGCCCTGTCCCG | | |
| | GGACAACATGCTGCCGGGCTCCAAGGTGCTGGCGAAGATCAACCCGCACTTCGGCACG | | |
| | CCGGTCGCCGCCATCGTGCTGATCACCGCCATCGCCGTGCTGCTGAACCTGGCGAGTG | | |
| | GCGGGTTTGTCACGGCGATCTACTCGATGGTCGGCCTGACCTACTACTGCACTTACCT | | |
| | GCTGACGCTGATTGCCGCGTACCTGGCCTATAAAAACGGCCGGATGCCGGGGGCGCCT | | |
| | GCGGGCGTGTTCAGCCTGGGCCGCTGGTTGCTGCCGATGATTATCCTCGGCGGCCTGT | | |
| | GGGCCATCGCGGTGATCCTGACCCTGAGCGTGCCGGAAGAAAGCCACACTGGCGCTAT | | |
| | CACCACCGGGGTTACACTCGGCGTGGGCGTGTTGTGGTGGTTGTTTTCACTGCGCACG | | |
| | CGCCTTAACAATGGCACCGCCGGGCCGAGCGGCAAATTGCTCGACCACTAGCCGCTGA | | |
| | TTGCAGCCAAAAGACAAAACCCCGAACACCGGGGTTTTGTCTTGTCACCTCCAAGGAG | | |
| | CTTCCCGATGTTTGAACAGGCCAGCTGGCTCAATCAACCCCAGCATTGGCGCCGAGAA | | |
| | GGCGAGCGACTCAAGGTCCGCACCGATGCCAGTACCGATTTCTGGCGTGAAACCCACT | | |
| | ATGGTTTTGTACGCGACAACGGGCATTTCCTGTTTGTTGAAACCGACGGCGACTTTAC | | |
| | CGCCCAAGTCAAAATCCACAGTGAGTTTACCCACCTGTATGACCTTCGC | | |
| | ORF Start: ATG at 43 | | ORF Stop: TAG at 1441 |
| | SEQ ID NO:316 | 466 aa | MW at 49070.4 kD |
| NOV113a, | MHRTMSSFTSFALAFSMVSINTGVVTLFADPFNRVGGIGILLWLLVIPLVCCIVMVYC | | |
| CG59887-01 Protein Sequence | | | |
| | HLAGRIPLTGYAYQWSSRLAGNHFGWFTGWVAFTSFVAGTAATSAAIGTVFAPEIWAN | | |
| | PTQGQIQGLSIGATLVVGLLNICGIRLATRINDIGAIIEIIGTVLLAIALFFGVFFFF | | |
| | EHTQGVAILTSAQPVSGGTLSFTTIALATLLPVSVLLGWEGAADLSEETKDPRRAAPR | | |
| | AMIRAVLVSSVLGFVVFALLSIAIPGSVSELLSHSENPVINIVRLQLGNAAGVGMIVI | | |
| | AFASILACLIANMAVATRMTFALSRDNMLPGSKVLAKINPHFGTPVAAIVLITAIAVL | | |
| | LNLASGGFVTAIYSMVGLTYYCTYLLTLIAAYLAYKNGRMPGAPAGVFSLGRWLLPMI | | |
| | ILGGLWAIAVILTLSVPEESHTGAITTGVTLGVGVLWWLFSLRTRLNNGTAGPSGKLL | | |
| | DH | | |
| | SEQ ID NO:317 | 1433 bp | |
| NOV113b, | AAAAATGCACCGCACCATGAGCTCGTTCACCTCGTTTGCCCTGGCCTTTTCCATGGTC | | |
| CG59887-02 DNA Sequence | | | |
| | TCGATCAACACCGGCGTGGTCACGCTGTTCGCCGACCCGTTCAACCGCGTCGGGGGCA | | |
| | TCGGCATCCTCCTGTGGCTGTTGGTGATCCCGCTGGTGTGCTGCATCGTCATGGTCTA | | |
| | CTGCCACCTGGCCGGGCGCATTCCGCTCACCGGCTACGCCTACCAATGGTCCAGCCGA | | |
| | TTGGCGGGCAATCACTTCGGCTGGTTTACCGGCTGGGTGGCGTTCACCTCGTTTGTCG | | |
| | CCGGTACAGCCGCCACCTCGGCGGCCATCGGTACGGTGTTCGCACCGGAGATCTGGGC | | |
| | CAACCCGACACAGGGTCAGATCCAGGGCCTGAGCATCGGCGCCACGCTGGTGGTGGGC | | |
| | TTGCTGAATATCTGCGGGATTCGCCTGGCCACCCGGATCAACGACATCGGCGCGATCA | | |
| | TCGAAATCATCGGCACGGTACTGCTGGCGATTGCGTTGTTCTTCGGGGTGTTTTTCTT | | |
| | CTTTGAGCACACCCAGGGCGTGGCGATCCTGACCTCCGCGCAACCAGTGAGCGGCGGC | | |
| | ACGCTCAGCTTCACCACCATCGCCCTCGCCACCTTGCTGCCGGTCTCGGTGCTGCTGG | | |
| | GTTGGGAAGGCGCCGCCGACCTGTCCGAGGAAACCAAGGACCCACGCCGCGCCGCGCC | | |
| | CCGGGCGATGATTCGTGCGGTGCTGGTGTCCAGCGTATTGGGCTTCGTGGTGTTCGCC | | |
| | TTGCTGAGCATCGCGATCCCGGGCTCGGTCAGCGAACTGCTCAGCCGCAGCGAAAACC | | |
| | CGGTGATCAATATCGTGCGCCTGCAACTGGGCAATGCCGCCGGCGTGGGCATGGTCGT | | |
| | GATCGCTTTCGCCTCGATCCTCGCCTGCCTGATCGCCAACATGGCGGTGGCCACGCGC | | |
| | ATGACCTTCGCCCTGTCCCGGGACAACATGCTGCCGGGCTCCAAGGTGCTGGCGAAGA | | |
| | TCAACCCGCACTTCGGCACGCCGGTCGCCGCCATCGTGCTGATCACCGCCATCGCCGT | | |
| | GCTGCTGAACCTGGCGAGTGGCGGGTTTGTCACGGCGATCTACTCGATGGTCGGCCTG | | |
| | ACCTACTACTGCACTTACCTGCTGACGCTGATTGCCGCGTACCTGGCCTATAAAAACG | | |
| | GCCGGATGCCGGGGGCGCCTGCGGGCGTGTTCAGCCTGGGCCGCTGGTTGCTGCCGAT | | |
| | GATTATCCTCGGCGGCCTGTGGGCCATCGCGGTGATCCTGACCCTGAGCGTGCCGGAA | | |
| | GAAAGCCACACTGGCGCTATCACCACCGGGGTTACACTCGGCGTGGGCGTGTTGTGGT | | |
| | GGTTGTTTTCACTGCGCACGCGCCTTAACAATGGCACCGCCGGGCCGAGCGGCAAATT | | |
| | GCTCGACCACTAGCCGCTGATTGCAGCCAAAAGACAAAACC | | |
| | ORF Start: ATG at 5 | | ORF Stop: TAG at 1403 |
| | SEQ ID NO:318 | 466 aa | MW at 49075.4 kD |

| | |
|---|---|
| NOV113b, | MHRTMSSFTSFALAFSMVSINTGVVTLFADPFNRVGGIGILLWLLVIPLVCCIVMVYC |
| CG59887-02 Protein Sequence | |
| | HLAGRIPLTGYAYQWSSRLAGNHFGWFTGWVAFTSFVAGTAATSAAIGTVFAPEIWAN |
| | PTQGQIQGLSIGATLVVGLLNICGIRLATRINDIGAIIEIIGTVLLAIALFFGVFFFF |
| | EHTQGVAILTSAQPVSGGTLSFTTIALATLLPVSVLLGWEGAADLSEETKDPRRAAPR |
| | AMIRAVLVSSVLGFVVFALLSIAIPGSVSELLSRSENPVINIVRLQLGNAAGVGMVVI |
| | AFASILACLIANMAVATRMTFALSRDNMLPGSKVLAKINPHFGTPVAAIVLITAIAVL |
| | LNLASGGFVTAIYSMVGLTYYCTYLLTLIAAYLAYKNGRMPGAPAGVFSLGRWLLPMI |
| | ILGGLWAIAVILTLSVPEESHTGAITTGVTLGVGVLWWLFSLRTRLNNGTAGPSGKLL |
| | DH |

Sequence comparison of the above protein sequences yields the following sequence relationships shown in Table 113B.

TABLE 113B
Comparison of NOV113a against NOV113b.

| | | Identities/ |
|---|---|---|
| | | Similarities |
| Protein | NOV113a Residues/ | for the |
| Sequence | Match Residues | Matched Region |
| NOV113b | 1 . . . 466 | 343/466 (73%) |
| | 1 . . . 466 | 344/466 (73%) |

Further analysis of the NOV113a protein yielded the following properties shown in Table 113C.

TABLE 113C
Protein Sequence Properties NOV113a

| | |
|---|---|
| PSort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic reticulum (lumen) |
| SignalP | Likely cleavage site between residues 59 and 60 |
| analysis: | |

A search of the NOV113a protein against the Geneseq database, a proprietary database that contains sequences published in patents and patent publication, yielded several homologous proteins shown in Table 113D.

TABLE 113D
Geneseq Results for NOV113a

| | | NOV113a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAG49885 | Arabidopsis thaliana protein | 1 . . . 449 | 122/486 (25%) | 3e−31 |
| | fragment SEQ ID NO: 63155 - | 17 . . . 492 | 217/486 (44%) | |
| | Arabidopsis thaliana, 504 aa. | | | |
| | [EP1033405-A2, 6 SEP 2000] | | | |
| AAG49884 | Arabidopsis thaliana protein | 1 . . . 449 | 122/486 (25%) | 3e−31 |
| | fragment SEQ ID NO: 63154 - | 29 . . . 504 | 217/486 (44%) | |
| | Arabidopsis thaliana, 516 aa. | | | |
| | [EP1033405-A2, 6 SEP 2000] | | | |
| AAG20282 | Arabidopsis thaliana protein | 1 . . . 449 | 122/486 (25%) | 3e−31 |
| | fragment SEQ ID NO: 22407 - | 17 . . . 492 | 217/486 (44%) | |
| | Arabidopsis thaliana, 504 aa. | | | |
| | [EP1033405-A2, 6 SEP 2000] | | | |
| AAG20281 | Arabidopsis thaliana protein | 1 . . . 449 | 122/486 (25%) | 3e−31 |
| | fragment SEQ ID NO: 22406 - | 29 . . . 504 | 217/486 (44%) | |
| | Arabidopsis thaliana, 516 aa. | | | |
| | [EP1033405-A2, 6 SEP 2000] | | | |
| AAG20280 | Arabidopsis thaliana protein | 1 . . . 449 | 122/486 (25%) | 3e−31 |
| | fragment SEQ ID NO: 22405 - | 41 . . . 516 | 217/486 (44%) | |
| | Arabidopsis thaliana, 528 aa. | | | |
| | [EP1033405-A2, 6 SEP 2000] | | | |

In a BLAST search of public sequence databases, the NOV113a protein was found to have homology to the proteins shown in the BLASTP data in Table 113E.

TABLE 113E
Public BLASTP Results for NOV113a

| | | NOV113a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9KZF1 | PROBABLE AMINO | 3 . . . 450 | 139/469 (29%) | 2e−41 |
| | ACID/METABOLITE | 27 . . . 481 | 214/469 (44%) | |
| | PERMEASE - Streptomyces | | | |
| | coelicolor, 504 aa. | | | |
| Q98H14 | AMINO ACID/METABOLITE | 1 . . . 446 | 118/466 (25%) | 1e−36 |
| | PERMEASE - Rhizobium loti | 27 . . . 485 | 209/466 (44%) | |
| | (Mesorhizobium loti), 518 aa. | | | |
| Q92NI8 | PUTATIVE AMINO-ACID | 1 . . . 449 | 122/475 (25%) | 1e−32 |
| | PERMEASE PROTEIN - | 25 . . . 487 | 204/475 (42%) | |
| | Rhizobium meliloti (Sinorhizobium | | | |
| | meliloti), 515 aa. | | | |
| O22509 | PUTATIVE AMINO ACID OR | 1 . . . 449 | 122/486 (25%) | 1e−30 |
| | GABA PERMEASE - Arabidopsis | 29 . . . 504 | 217/486 (44%) | |
| | thaliana (Mouse-ear cress), 516 aa. | | | |
| Q9ZU50 | PUTATIVE AMINO ACID | 1 . . . 449 | 120/487 (24%) | 2e−28 |
| | PERMEASE - Arabidopsis thaliana | 29 . . . 505 | 216/487 (43%) | |
| | (Mouse-ear cress), 517 aa. | | | |

PFam analysis predicts that the NOV113a protein contains the domains shown in the Table 113F.

TABLE 113F
Domain Analysis of NOV113a

| | | Identities/ | | |
|---|---|---|---|---|
| | | Similarities | | |
| | NOV113a | for the | | Expect |
| Pfam Domain | Match Region | Matched Region | | Value |
| oxidored_q3: | 162 . . . 307 | 28/182 | (15%) | 3.7 |
| domain 1 of 1 | | 91/182 | (50%) | |
| ISK_Channel: | 196 . . . 326 | 32/136 | (24%) | 8.8 |
| domain 1 of 1 | | 55/136 | (40%) | |
| ABC2_membrane: | 122 . . . 377 | 46/273 | (17%) | 8.3 |
| domain 1 of 1 | | 154/273 | (56%) | |
| SSF: domain 1 of 1 | 7 . . . 394 | 77/470 | (16%) | 7.8 |
| | | 222/470 | (47%) | |
| Aa_trans: | 29 . . . 417 | 67/483 | (14%) | 9.7 |
| domain 1 of 1 | | 236/483 | (49%) | |
| aa_permeases: | 1 . . . 451 | 86/516 | (17%) | 1.1e−05 |
| domain 1 of 1 | | 287/516 | (56%) | |

The NOV114 clone was analyzed, and the nucleotide and predicted polypeptide sequences are shown in Table 114A.

TABLE 114A
NOV114 Sequence Analysis

| | SEQ ID NO:319 | 876 bp | |
|---|---|---|---|
| NOV114a, | AACTTGCTTTTGGGAGCCAGCGGTATGGCGTCGGGCTGCAAGATTGGCCCGTCCATCC | | |
| CG59861-01 DNA Sequence | | | |
| | TCAACAGCGACCTGGCCAATTTAGGGGCCGAGTGCTCCCGGATGCTAGACTCTGGGGC | | |
| | CGATTATCTGCACCTGGACGTAATGGACGGGCATTTTGTTCCCAACATCACCTTTGGT | | |
| | CACCCTGTGGTGGAAAGCCTTCGAAAGCAGCTAGGCCAGGACCCTTTCTTTGACATGC | | |
| | ACATGATGGTGTCCAAGCCAGAACAGTGGGTAAAGCCAATGGCTGTAGCAGGAGCCAA | | |
| | TCAGTACACCTTTCATCTCGAGGCTACTGAGAACCCAGGGGCTTTGATTAAAGACATT | | |
| | CGGGAGAATGGGATGAAGGTTGGCCTTGCCATCAAACCAGGAACCTCAGTTGAGTATT | | |
| | TGGCACCATGGGCTAATCAGATAGATATGGCCTTGGTTATGACAGTGGAACCGGGGTT | | |
| | TGGAGGGCAGAAATTCATGGAAGATATGATGCCAAAGGTTCACTGGTTGAGGACCCAG | | |
| | TTCCCATCTTTGGATATAGAGGTCGATGGTGGAGTAGGTCCTGACACTGTCCATAAAT | | |
| | GTGCAGAGGCAGGAGCTAACATGATTGTGTCTGGCAGTGCTATTATGAGGAGTGAAGA | | |
| | CCCCAGATCTGTGATCAATCTATTAAGAAATGTTTGCTCAGAAGCTGCTCAGAAACGT | | |
| | TCTCTTGATCGGTGAAACCATAAGGAGCCCAGTGTTCCTGTTCATGAAATCTCCCTTT | | |
| | TACTGGAAAACAGGAATATTGACTACCAAATCACAATGCAATTGAAGCCGTACTGCTT | | |
| | TTTTGAGCAGTTATTCATTCCAGTGATTAAAACTGATTGTGCAGAATAAAAAAAAAAA | | |
| | AAAAAA | | |
| | ORF Start: ATG at 25 | | ORF Stop: TGA at 709 |
| | SEQ ID NO:320 | 228 aa | MW at 24901.4 kD |
| NOV114a, | MASGCKIGPSILNSDLANLGAECSRMLDSGADYLHLDVMDGHFVPNITFGHPVVESLR | | |
| CG59861-01 Protein Sequence | | | |
| | KQLGQDPFFDMHMMVSKPEQWVKPMAVAGANQYTFHLEATENPGALIKDIRENGMKVG | | |
| | LAIKPGTSVEYLAPWANQIDMALVMTVEPGFGGQKFMEDMMPKVHWLRTQFPSLDIEV | | |
| | DGGVGPDTVHKCAEAGANMIVSGSAIMRSEDPRSVINLLRNVCSEAAQKRSLDR | | |
| | SEQ ID NO:321 | 730 bp | |
| NOV114b, | AACTTGCTTTTGGGAGCCAGCGGTATGGCGTCGGGCTGCAAGATTGGCCCGTCCATCC | | |
| CG59861-02 DNA Sequence | | | |
| | TCAACAGCGACCTGGCCAATTTAGGGGCCGAGTGCCTCCGGATGCTAGACTCTGGGGC | | |
| | CGATTATCTGCACCTGGACGTAATGGACGGGCATTTTGTTCCCAACATCACCTTTGGT | | |
| | CACCCTGTGGTAGAAAGCCTTCGAAAGCAGCTAGGCCAGGACCCTTTCTTTGACATGC | | |
| | ACATGATGGTGTCCAAGCCAGAACAGTGGGTAAAGCCAATGGCTGTAGCAGGAGCCAA | | |
| | TCAGTACACCTTTCATCTCGAGGCTACTGAGAACCCAGGGGCTTTGATTAAAGACATT | | |
| | CGGGAGAATGGGATGAAGGTTGGCCTTGCCATCAAACCAGGAACCTCAGTTGAGTATT | | |
| | TGGCACCATGGGCTAATCAGATAGATATGGCCTTGGTTATGACAGTGGAACCGGGGTT | | |
| | TGGAGGGCAGAAATTCATGGAAGATATGATGCCAAAGGTTCACTGGTTGAGGACCCAG | | |
| | TTCCCATCTTTGGATATAGAGGTCGATGGTGGAGTAGGTCCTGACACTGTCCATAAAT | | |
| | GTGCAGAGGCAGGAGCTAACATGATTGTGTCTGGCAGTGCTATTATGAGGAGTGAAGA | | |
| | CCCCAGATCTGTGATCAATCTATTAAGAAATGTTTGCTCAGAAGCTGCTCAGAAACGT | | |
| | TCTCTTGATCGGTGAAACCATAAGGAGCCCAGTG | | |
| | ORF Start: ATG at 25 | | ORF Stop: TGA at 709 |
| | SEQ ID NO:322 | 228 aa | MW at 24927.5 kD |

| | |
|---|---|
| NOV114b, | MASGCKIGPSILNSDLANLGAECLRMLDSGADYLHLDVMDGHFVPNITFGHPVVESLR |
| CG59861-02 Protein Sequence | |
| | KQLGQDPFFDMHMMVSKPEQWVKPMAVAGANQYTFHLEATENPGALIKDIRENGMKVG |
| | LAIKPGTSVEYLAPWANQIDMALVMTVEPGFGGQKFMEDMMPKVHWLRTQFPSLDIEV |
| | DGGVGPDTVHKCAEAGANMIVSGSAIMRSEDPRSVINLLRNVCSEAAQKRSLDR |

Sequence comparison of the above protein sequences yields the following sequence relationships shown in Table 114B.

TABLE 114B
Comparison of NOV114a against NOV114b.

| | | Identities/ |
|---|---|---|
| | | Similarities |
| Protein | NOV114a Residues/ | for the |
| Sequence | Match Residues | Matched Region |
| NOV114b | 1 . . . 228 | 227/228 (99%) |
| | 1 . . . 228 | 227/228 (99%) |

Further analysis of the NOV114a protein yielded the following properties shown in Table 114C.

TABLE 114C
Protein Sequence Properties NOV114a

| | |
|---|---|
| PSort | 0.6500 probability located in cytoplasm; 0.1753 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV114a protein against the Geneseq database, a proprietary database that contains sequences published in patents and patent publication, yielded several homologous proteins shown in Table 114D.

TABLE 114D
Geneseq Results for NOV114a

| | Protein/Organism/Length | NOV114a | Identities/ | | |
|---|---|---|---|---|---|
| | [Patent #, | Residues/ | Similarities | | |
| Geneseq | Protein/Organism/Length | Match | for the | | Expect |
| Identifier | [Patent #, Date] | Residues | Matched Region | | Value |
| AAM41358 | Human polypeptide SEQ ID NO | 1 . . . 228 | 227/228 | (99%) | e−132 |
| | 6289 - Homo sapiens, 247 aa. | 20 . . . 247 | 227/228 | (99%) | |
| | [WO200153312-A1, 26 JUL | | | | |
| | 2001] | | | | |
| AAM41357 | Human polypeptide SEQ ID NO | 1 . . . 228 | 227/228 | (99%) | e−132 |
| | 6288 - Homo sapiens, 247 aa. | 20 . . . 247 | 227/228 | (99%) | |
| | [WO200153312-A1, 26 JUL | | | | |
| | 2001] | | | | |
| AAM39571 | Human polypeptide SEQ ID NO | 1 . . . 228 | 227/228 | (99%) | e−132 |
| | 2716 - Homo sapiens, 228 aa. | 1 . . . 228 | 227/228 | (99%) | |
| | [WO200153312-A1, 26 JUL | | | | |
| | 2001] | | | | |
| AAB71912 | Human ISOM-4 - Homo sapiens, | 1 . . . 228 | 227/228 | (99%) | e−132 |
| | 228 aa. [WO200112790-A2, 22 | 1 . . . 228 | 227/228 | (99%) | |
| | FEB 2001] | | | | |
| AAM39572 | Human polypeptide SEQ ID NO | 1 . . . 228 | 227/246 | (92%) | e−129 |
| | 2717 - Homo sapiens, 246 aa. | 1 . . . 246 | 227/246 | (92%) | |
| | [WO200153312-A1, 26 JUL | | | | |
| | 2001] | | | | |

In a BLAST search of public sequence databases, the NOV114a protein was found to have homology to the proteins shown in the BLASTP data in Table 114E.

TABLE 114E
Public BLASTP Results for NOV114a

| | | NOV114a | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| Q96AT9 | HYPOTHETICAL 24.9 KDA | 1 . . . 228 | 227/228 | (99%) | e−131 |
| | PROTEIN - Homo sapiens | 1 . . . 228 | 227/228 | (99%) | |
| | (Human), 228 aa. | | | | |
| Q9BSB5 | HYPOTHETICAL 25.3 KDA | 1 . . . 228 | 227/228 | (99%) | e−131 |
| | PROTEIN- Homo sapiens | 5 . . . 232 | 227/228 | (99%) | |
| | (Human), 232 aa (fragment). | | | | |
| AAH19126 | HYPOTHETICAL 24.9 KDA | 1 . . . 228 | 221/228 | (96%) | e−129 |
| | PROTEIN - Mus musculus | 1 . . . 228 | 226/228 | (98%) | |
| | (Mouse), 228 aa. | | | | |
| O43767 | RIBULOSE-5-PHOSPHATE- | 55 . . . 228 | 174/174 | (100%) | 2e−98 |
| | EPIMERASE - Homo sapiens | 1 . . . 174 | 174/174 | (100%) | |
| | (Human), 174 aa (fragment). | | | | |
| Q96N34 | CDNA FLJ31466 FIS, CLONE | 69 . . . 228 | 160/178 | (89%) | 2e−86 |
| | NT2NE2001372, HIGHLY | 1 . . . 178 | 160/178 | (89%) | |
| | SIMILAR TO HOMO SAPIENS | | | | |
| | PUTATIVE RIBULOSE-5- | | | | |
| | PHOSPHATE-EPIMERASE - | | | | |
| | Homo sapiens (Human), 178 aa. | | | | |

PFam analysis predicts that the NOV114a protein contains the domains shown in the Table 114F.

TABLE 114F
Domain Analysis of NOV114a

| | | Identities/ | | |
|---|---|---|---|---|
| | | Similarities | | |
| Pfam | NOV114a | for the | | Expect |
| Domain | Match Region | Matched Region | | Value |
| Ribul_P_3_epim: | 6 . . . 204 | 95/209 | (45%) | 1.9e−105 |
| domain 1 of 1 | | 174/209 | (83%) | |
| IGPS: domain 1 of 1 | 179 . . . 213 | 15/35 | (43%) | 0.02 |
| | | 27/35 | (77%) | |
| trp_syntA: | 34 . . . 222 | 45/273 | (16%) | 2.9 |
| domain 1 of 1 | | 124/273 | (45%) | |

The NOV115 clone was analyzed, and the nucleotide and predicted polypeptide sequences are shown in Table 115A.

TABLE 115A
NOV115 Sequence Analysis

| | SEQ ID NO:323 | 1761 bp | |
|---|---|---|---|
| NOV115a, | AGTGTGGTACCTATCTGTCCCCCCTCTGGAGGGGTTGACAAGGGAAAGGGCACCGGGG | | |
| CG59857-01 DNA Sequence | | | |
| | GGCACAGAGATGCAGGACAGATTGCACATCCTGGAGGACCTGAATATGCTCTACATTC | | |
| | GGCAGATGGCACTCAGCCTGGAGGACACGGAGTTGCAGAGGAAGCTAGACCATGAGAT | | |
| | CCGGATGAGGGAAGGGGCCTGTAAGCTGCTGGCAGCCTGCTCCCAGCGAGAGCAGGCT | | |
| | CTGGAGGCCACCAAGAGCCTGCTAGTGTGCAACAGCCGCATCCTCAGCTACATGGGCG | | |
| | AGCTGCAGCGGCGCAAGGAGGCGCAGGTGCTGGGGAAGACAAGCCGGCGGCCTTCTGA | | |
| | CAGTGGCCCGCCCGCTGAGCGCTCCCCCTGCCGCGGCCGGGTCTGCATCTCTGACCTC | | |
| | CGGATTCCACTCATGTGGAAGGACACAGAATATTTCAAGAACAAAGACTTGCACCGCT | | |
| | GGGCTGTGTTCCTGCTGCTGCAGCTGGGGGAACACATCCAGGACACAGAGATGATCCT | | |
| | AGTGGACAGGACCCTCACAGACATCTCCTTTCAGAGCAATGTGCTCTTCGCTGAGGCG | | |
| | GGGCCAGACTTTGAACTGCGGTTAGAGCTGTATGGGGCCTGTGTGGAAGAAGAGGGGG | | |
| | CCCTGACTGGCGGCCCCAAGAGGCTTGCCACCAAACTCAGCAGCTCCCTGGGCCGCTC | | |
| | CTCAGGGAGGCGTGTCCGGGCATCGCTGGACAGTGCTGGGGGTTCAGGGAGCAGTCCC | | |
| | ATCTTGCTCCCCACCCCAGTTGTTGGTGGTCCTCGTTACCACCTCTTGGCTCACACCA | | |
| | CACTCACCCTGGCAGCAGTGCAAGATGGATTCCGCACACATGACCTCACCCTTGCCAG | | |
| | TCATGAGGAGAACCCTGCCTGGCTGCCCCTTTATGGTAGCGTGTGTTGCCGTCTGGCA | | |
| | GCTCAGCCTCTCTGCATGACTCAGCCCACTGCAAGTGGTACCCTCAGGGTGCAGCAAG | | |
| | CTGGGGAGATGCAGAACTGGGCACAAGTGCATGGAGTTCTGAAAGGCACAAACCTCTT | | |
| | CTGTTACCGGCAACCTGAGGATGCAGACACTGGGGAAGAGCCGCTGCTTACTATTGCT | | |
| | GTCAACAAGGAGACTCGAGTCCGGGCAGGGGAGCTGGACCAGGCTCTAGGACGGCCCT | | |
| | TCACCCTAAGCATCAGTAACCAGTATGGGGATGATGAGGTGACACACACCCTTCAGAC | | |
| | AGAAAGTCGGGAAGCACTGCAGAGCTGGATGGAGGCTCTGTGGCAGCTTTTCTTTGAC | | |
| | ATGAGCCAATGGAAGCAGTGCTGTGATGAAATCATGAAAATTGAAACTCCTGCTCCCC | | |
| | GGAAACCACCCCAAGCACTGGCAAAGCAGGGGTCCTTGTACCATGAGATGGCTATTGA | | |
| | GCCGCTGGATGACATCGCAGCGGTGACAGACATCCTGACCCAGCGGGAGGGCGCAAGG | | |
| | CTGGAGACACCCCCACCCTGGCTGGCAATGTTTACAGACCAGCCTGCCCTGCCTAACC | | |
| | CCTGCTCGCCTGCCTCAGTGGCCCCAGCCCCAGACTGGACCCACCCCCTGCCCTGGGG | | |
| | GAGACCCCGAACCTTTTCCCTGGATGCTGTCCCCCCAGACCACTCCCCTAGGGCTCGC | | |
| | TCGGTTGCCCCCCTCCCACCTCAGCGATCCCCACGGACCAGAGGCCTCTGCAGCAAAG | | |
| | GCCAACCTCGCACTTGGCTCCAGTCACCAGTGTGAGAGAGAAAGGTGCTGGCATAGGA | | |
| | TCTGCCCAGAAGAGAAAATGA | | |
| | ORF Start: ATG at 68 | | ORF Stop: TGA at 1715 |
| | SEQ ID NO:324 | 549 aa | MW at 61171.0 kD |

| | |
|---|---|
| NOV115a, | MQDRLHILEDLNMLYIRQMALSLEDTELQRKLDHEIRMREGACKLLAACSQREQALEA |
| CG5987-01 Protein Sequence | |
| | TKSLLVCNSRILSYMGELQRRKEAQVLGKTSRRPSDSGPPAERSPCRGRVCISDLRIP |
| | LMWKDTEYFKNKDLHRWAVFLLLQLGEHIQDTEMILVDRTLTDISFQSNVLFAEAGPD |
| | FELRLELYGACVEEEGALTGGPKRLATKLSSSLGRSSGRRVRASLDSAGGSGSSPILL |
| | PTPVVGGPRYHLLAHTTLTLAAVQDGFRTHDLTLASHEENPAWLPLYGSVCCRLAAQP |
| | LCMTQPTASGTLRVQQAGEMQNWAQVHGVLKGTNLFCYRQPEDADTGEEPLLTIAVNK |
| | ETRVRAGELDQALGRPFTLSISNQYGDDEVTHTLQTESREALQSWMEALWQLFFDMSQ |
| | WKQCCDEIMKIETPAPRKPPQALAKQGSLYHEMAIEPLDDIAAVTDILTQREGARLET |
| | PPPWLAMFTDQPALPNPCSPASVAPAPDWTHPLPWGRPRTFSLDAVPPDHSPRARSVA |
| | PLPPQRSPRTRGLCSKGQPRTWLQSPV |

Further analysis of the NOV115a protein yielded the following properties shown in Table 115B.

TABLE 115B
Protein Sequence Properties NOV115a

| | |
|---|---|
| PSort | 0.4500 probability located in cytoplasm; 0.3000 |
| analysis: | probability located in microbody (peroxisome); |
| | 0.1707 probability located in lysosome (lumen); |
| | 0.1000 probability located in mitochondrial |
| | matrix space |
| SignalP | No Known Signal Sequence Predicted |
| analysis: | |

A search of the NOV115a protein against the Geneseq database, a proprietary database that contains sequences published in patents and patent publication, yielded several homologous proteins shown in Table 115C.

TABLE 115C
Geneseq Results for NOV115a

| | | NOV115a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB35241 | Human rhotekin - Homo sapiens, | 24 . . . 549 | 526/527 (99%) | 0.0 |
| | 563 aa. [US6183990-B1, 6 FEB | 37 . . . 563 | 526/527 (99%) | |
| | 2001] | | | |
| AAY44559 | Human Rhotekin protein - Homo | 24 . . . 549 | 526/527 (99%) | 0.0 |
| | sapiens, 563 aa. [WO9958667-A1, | 37 . . . 563 | 526/527 (99%) | |
| | 18 NOV 1999] | | | |
| AAB35242 | Human rhotekin EST-derived | 24 . . . 549 | 522/527 (99%) | 0.0 |
| | protein - Homo sapiens, 527 aa. | 1 . . . 527 | 523/527 (99%) | |
| | [US6183990-B1, 6 FEB 2001] | | | |
| AAY44560 | Human Rhotekin variant protein - | 24 . . . 549 | 522/527 (99%) | 0.0 |
| | Homo sapiens, 527 aa. | 1 . . . 527 | 523/527 (99%) | |
| | [WO9958667-A1, 18 NOV 1999] | | | |
| AAB26790 | Human Ras correlative GTP | 24 . . . 549 | 518/527 (98%) | 0.0 |
| | binding kinase protein sequence - | 18 . . . 544 | 519/527 (98%) | |
| | Homo sapiens, 544 aa. | | | |
| | [CN1257924-A, 28 JUN 2000] | | | |

In a BLAST search of public sequence databases, the NOV115a protein was found to have homology to the proteins shown in the BLASTP data in Table 115D.

TABLE 115D
Public BLASTP Results for NOV115a

| | | NOV115a | Identities/ | |
|---|---|---|---|---|
| Protein | | Residues/ | Similarities | |
| Accession | | Match | for the | Expect |
| Number | Protein/Organism/Length | Residues | Matched Portion | Value |
| AAH17727 | SIMILAR TO RHOTEKIN - | 1 . . . 549 | 549/550 (99%) | 0.0 |
| | Homo sapiens (Human), 550 aa. | 1 . . . 550 | 549/550 (99%) | |
| Q9BST9 | SIMILAR TO RHOTEKIN - | 24 . . . 549 | 526/527 (99%) | 0.0 |
| | Homo sapiens (Human), 587 aa | 61 . . . 587 | 526/527 (99%) | |
| | (fragment). | | | |
| Q96PT6 | RTKN - Homo sapiens (Human), | 24 . . . 549 | 518/527 (98%) | 0.0 |
| | 544 aa. | 18 . . . 544 | 519/527 (98%) | |
| Q9HB05 | RHOTEKIN - Homo sapiens | 24 . . . 549 | 505/527 (95%) | 0.0 |
| | (Human), 567 aa (fragment). | 41 . . . 567 | 513/527 (96%) | |
| Q61192 | RHOTEKIN - Mus musculus | 1 . . . 549 | 477/551 (86%) | 0.0 |
| | (Mouse), 551 aa. | 1 . . . 551 | 500/551 (90%) | |

PFam analysis predicts that the NOV115a protein contains the domains shown in the Table 115E.

TABLE 115E
Domain Analysis of NOV115a

| | | Identities/ | | |
|---|---|---|---|---|
| | | Similarities | | |
| Pfam | NOV115a | for the | | Expect |
| Domain | Match Region | Matched Region | | Value |
| HR1: domain 1 of 1 | 23 . . . 95 | 17/87 | (20%) | 0.27 |
| | | 54/87 | (62%) | |
| PH: domain 1 of 1 | 296 . . . 397 | 19/102 | (19%) | 1e−06 |
| | | 72/102 | (71%) | |

### Example 116

The NOV116 clone was analyzed, and the nucleotide and predicted polypeptide sequences are shown in Table 116A.

TABLE 116A
NOV116 Sequence Analysis

| | SEQ ID NO:325 | 450 bp | |
|---|---|---|---|
| NOV116a, | CTGGGAGACTGAAAAAATGCAGACCACCGGGGTATTACTCATTTCTCCAGCTCTGATC | | |
| CG59855-01 DNA Sequence | | | |
| | TGCTGTTGTACCAGGGGTCTAATCAGGCCTGTGTCTGCCTTCTCCTTGAATAGCCCAG | | |
| | AGAATTCATCTAAACAGCCTTCCTACAGCAGCTCCCCACTCCAGGTGGCCAGACGGGA | | |
| | GTTCCAGACCAGTGTTGTCTCCCGGGACACTGACACAGCCGCCAAGTTTATTGGTGCT | | |
| | GGGTCAGCCACAGTTGGTGTGGCTGATTCAGGGGCTGGCATTGGAGCGGTGTTTGGCA | | |
| | GCTTGATTATTGTCTATGCCAGGAAGCTGTCTCTCAAGCAGCAACTCCTCTTCTATGC | | |
| | CATTCTGGGCTTTGCCCTGTCTGAGGCCATGGGGCTCTTCTGTTTGATGATCTCCTTC | | |
| | TTCATCCTGTTCGCCATGTGAGGCTCCGTGAGGGTCACCTGCCT | | |
| | ORF Start: ATG at 17 | | ORF Stop: TGA at 425 |
| | SEQ ID NO:326 | 136 aa | MW at 14384.6 kD |
| NOV116a, | MQTTGVLLISPALICCCTRGLIRPVSAFSLNSPENSSKQPSYSSSPLQVARREFQTSV | | |
| CG59855-01 Protein Sequence | | | |
| | VSRDTDTAAKFIGAGSATVGVADSGAGIGAVFGSLIIVYARKLSLKQQLLFYAILGFA | | |
| | LSEAMGLFCLMISFFILFAM | | |
| | SEQ ID NO:327 | 434 bp | |
| NOV116b, | ATGCAGACCACCGGGGTATTACTCATTTCTCCAGCTCTGATCTGCTGTTGTACCAGGG | | |
| CG59855-02 DNA Sequence | | | |
| | GTCTAATCAGGCCTGTGTCTGCCTTCTCCTTGAATAGCCCAGAGAATTCATCTAAACA | | |
| | GCCTTCCTACAGCAGCTCCCCACTCCAGGTGGCCAGACGGGAGTTCCAGACCAGTGTT | | |
| | GTCTCCCGGGACACTGACACAGCCGCCAAGTTTATTGGTGCTGGGTCAGCCACAGTTG | | |
| | GTGTGGCTGATTCAGAGGCTGGCATTGGAGCGGTGTTTGGCAGCTTGATTATTGTCTA | | |
| | TGCCAGGAAGCTGTCTCTCAAGCAGCAACTCCTCTTCTATGCCATTCTGGGCTTTGCC | | |
| | CTGTCTGAGGCCATGGGGCTCTTCTGTTTGATGATCTCCTTCTTCATCCTGTTCGCCA | | |
| | TGTGAGGCTCCGTGAGGGTCACCTGCCT | | |
| | ORF Start: ATG AT 1 | | ORF Stop: TGA at 409 |
| | SEQ ID NO:328 | 136 aa | MW at 14456.7 kD |

| | |
|---|---|
| NOV116b, | MQTTGVLLISPALICCCTRGLIRPVSAFSLNSPENSSKQPSYSSSPLQVARREFQTSV |
| CG59855-02 Protein Sequence | |
| | VSRDTDTAAKFIGAGSATVGVADSEAGIGAVFGSLIIVYARKLSLKQQLLFYAILGFA |
| | LSEAMGLFCLMISFFILFAM |

Sequence comparison of the above protein sequences yields the following sequence relationships shown in Table 116B.

TABLE 116B
Comparison of NOV116a against NOV116b.

| | | Identities/ |
|---|---|---|
| | | Similarities |
| Protein | NOV116a Residues/ | for the |
| Sequence | Match Residues | Matched Region |
| NOV116b | 1 . . . 136 | 120/136 (88%) |
| | 1 . . . 136 | 120/136 (88%) |

Further analysis of the NOV116a protein yielded the following properties shown in Table 116C.

TABLE 116C
Protein Sequence Properties NOV116a

| | |
|---|---|
| PSort | 0.9190 probability located in plasma membrane; 0.3000 |
| analysis: | probability located in lysosome (membrane); 0.1888 |
| | probability located in microbody (peroxisome); 0.1000 |
| | probability located in endoplasmic reticulum (membrane) |
| SignalP | Likely cleavage site between residues 28 and 29 |
| analysis: | |

A search of the NOV116a protein against the Geneseq database, a proprietary database that contains sequences published in patents and patent publication, yielded several homologous proteins shown in Table 116D.

TABLE 116D
Geneseq Results for NOV116a

| | | NOV116a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAG75142 | Human colon cancer antigen protein | 1 . . . 136 | 115/136 | (84%) | 2e−57 |
| | SEQ ID NO:5906 - Homo sapiens, | 7 . . . 142 | 119/136 | (86%) | |
| | 142 aa. [WO200122920-A2, | | | | |
| | 5 APR 2001] | | | | |
| AAB43866 | Human cancer associated protein | 1 . . . 136 | 115/136 | (84%) | 2e−57 |
| | sequence SEQ ID NO:1311 - Homo | 7 . . . 142 | 119/136 | (86%) | |
| | sapiens, 142 aa. [WO200055350- | | | | |
| | Al, 21 SEP 2000] | | | | |
| AAU69713 | Cell death protective sequence CNI- | 7 . . . 136 | 85/136 | (62%) | 2e−36 |
| | 00730, protein #1 - Homo sapiens, | 7 . . . 142 | 98/136 | (71%) | |
| | 142 aa. [WO200176532-A2, | | | | |
| | 18 OCT 2001] | | | | |
| ABB12016 | Human ATP synthase subunit | 7 . . . 136 | 85/136 | (62%) | 2e−36 |
| | homologue, SEQ ID NO:2386 - | 52 . . . 187 | 98/136 | (71%) | |
| | Homo sapiens, 187 aa. | | | | |
| | [WO200157188-A2, 9 AUG | | | | |
| | 2001] | | | | |
| AAB53428 | Human colon cancer antigen protein | 7 . . . 136 | 85/136 | (62%) | 2e−36 |
| | sequence SEQ ID NO:968 - Homo | 77 . . . 212 | 98/136 | (71%) | |
| | sapiens, 212 aa. [WO200055351 - | | | | |
| | A1, 21 SEP 2000] | | | | |

In a BLAST search of public sequence databases, the NOV116a protein was found to have homology to the proteins shown in the BLASTP data in Table 116E.

TABLE 116E
Public BLASTP Results for NOV116a

| | | NOV115a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P05496 | ATP synthase lipid-binding protein, | 1 . . . 136 | 115/136 (84%) | 9e−57 |
| | mitochondrial precursor (EC | 1 . . . 136 | 119/136 (86%) | |
| | 3.6.1.34) (ATP synthase proteolipid | | | |
| | P1) (ATPase protein 9) (ATPase | | | |
| | subunit C) - Homo sapiens (Human), | | | |
| | 136 aa. | | | |
| P32876 | ATP synthase lipid-binding protein, | 1 . . . 136 | 113/136 (83%) | 1e−54 |
| | mitochondrial precursor (EC | 1 . . . 136 | 117/136 (85%) | |
| | 3.6.1.34) (ATP synthase proteolipid | | | |
| | P1) (ATPase protein 9) (ATPase | | | |
| | subunit C) - Bos taurus (Bovine), 136 | | | |
| | aa. | | | |
| P17605 | ATP synthase lipid-binding protein, | 1 . . . 136 | 113/136 (83%) | 2e−54 |
| | mitochondrial precursor (EC | 1 . . . 136 | 117/136 (85%) | |
| | 3.6.1.34) (ATP synthase proteolipid | | | |
| | P1) (ATPase protein 9) (ATPase | | | |
| | subunit C) - Ovis aries (Sheep), 136 | | | |
| | aa. | | | |
| Q9CR84 | ATP SYNTHASE C CHAIN | 1 . . . 136 | 112/136 (82%) | 1e−53 |
| | ISOFORM 1 (EC 3.6.1.34) (LIPID- | 1 . . . 136 | 117/136 (85%) | |
| | BINDING PROTEIN) (SUBUNIT C) - | | | |
| | Mus musculus (Mouse), 136 aa. | | | |
| P48202 | ATP synthase lipid-binding protein, | 1 . . . 136 | 112/136 (82%) | 1e−53 |
| | mitochondrial precursor (EC | 1 . . . 136 | 117/136 (85%) | |
| | 3.6.1.34) (ATP synthase proteolipid | | | |
| | P1) (ATPase protein 9) (ATPase | | | |
| | subunit C) - Mus musculus (Mouse), | | | |
| | 136 aa. | | | |

PFam analysis predicts that the NOV116a protein contains the domains shown in the Table 116F.

TABLE 116F
Domain Analysis of NOV116a

| | | Identities/ | |
|---|---|---|---|
| | | Similarities | |
| Pfam | NOV116a | for the | Expect |
| Domain | Match Region | Matched Region | Value |
| ATP-synt_C: | 67 . . . 135 | 31/70 (44%) | 2.3e−18 |
| domain 1 of 1 | | 57/70 (81%) | |

The NOV117 clone was analyzed, and the nucleotide and predicted polypeptide sequences are shown in Table 117A.

TABLE 117A
NOV117 Sequence Analysis

| | SEQ ID NO:329 | 1769 bp | |
|---|---|---|---|
| NOV117a, | GAGGTGATGCTGGAGACCTGCGGACTTCTCATGTCTCTGGGCTGTCCTTTGTTCAAAC | | |
| CG59807-01 DNA Sequence | | | |
| | CAGAGCTGATCTACCAGTTGGATCACAGACAGGAGCTATGGATGGCTACAAAAGACCT | | |
| | CTCCCAAAGCTCCTATCCAGGTGACAACACAAAACCCAAGACCACAGAGCCTACCTTT | | |
| | TCTCACCTGGCCTTGCCTGAGGAAGTCTTACTCCAGGAACAACTGACACAAGGAGCCT | | |
| | CAAAGAACTCCCAATTAGGGCAATCCAAGGATCAGGATGGGCCATCTGAAATGCAAGA | | |
| | AGTCCACTTGAAAATAGGGATAGGCCCCCAGCGGGGGAAGCTGCTGGAGAAAATGAGT | | |
| | TCTGAACGTGATGGTTTGGGGTCAGATGATGGTGTATGTACAAAGATTACACAGAAAC | | |
| | AAGTTTCAACAGAAGGTGATCTCTATGAATGTGATTCACATGGACCAGTTACAGATGC | | |
| | CTTGATTCGCGAAGAGAAAAATTCCTATAAATGTGAGGAATGCGGGAAAGTGTTTAAA | | |
| | AAGAATGCCCTCCTTGTTCAGCATGAACGGATTCACACTCAAGTGAAGCCCTATGAAT | | |
| | GCACAGAGTGTGGGAAAACCTTTAGCAAGAGCACTCATCTTCTTCAGCACCTCATCAT | | |
| | CCACACTGGGGAGAACCCCTATAAGTGCATGGAGTGTGGGAAGGCTTTTAACCGCAGG | | |
| | TCACACCTCACACGGCACCAGCGGATTCACAGTGGAGAGAAGCCTTATAAGTGCAGTG | | |
| | AATGTGGAAAGGCCTTCACCCACCGCTCCACTTTTGTCTTGCATCACAGGAGCCACAC | | |
| | TGGAGAAAAACCCTTTGTGTGCAAAGAGTGTGGCAAAGCCTTTCGAGATAGGCCAGGT | | |
| | TTCATTCGACACTACATCATCCACACGGGAGAGAAGCCCTATGAGTGCATTGAGTGTG | | |
| | GGAAGGCCTTCAACCGCCGGTCATACCTCACGTGGCACCAACAGATTCACACTGGAGT | | |
| | GAAACCCTTTGAATGCAACGAGTGTGGAAAAGCTTTTTGCGAGAGTGCAGACCTCATT | | |
| | CAACACTACATTATCCACACTGGGGAGAAGCCCTATAAGTGCATGGAGTGTGGGAAGG | | |
| | CGTTCAACCGTAGGTCACACCTCAAGCAGCATCAACGGATTCACACTGGGGAGAAGCC | | |
| | TTATGAATGCAGTGAATGTGGAAAGGCCTTCACCCACTGCTCCACTTTTGTCTTGCAT | | |
| | AAAAGGACCCACACAGGAGAAAAACCCTATGAATGCAAAGAATGTGGAAAAGCCTTTA | | |
| | GTGATAGGGCAGACCTCATTCGCCACTTCAGCATCCACACTGGAGAGAAACCCTATGA | | |
| | GTGCGTGGAGTGTGGAAAGGCCTTCAACCGCAGCTCACACCTCACGAGGCACCAACAG | | |
| | ATTCACACTGGAGAGAAACCCTATGAATGCATCCAGTGTGGGAAAGCCTTTTGCCGGA | | |
| | GCGCAAACCTTATTCGACACTCCATCATTCACACTGGAGAGAAGCCGTATGAATGCAG | | |
| | TGAGTGTGGAAAGGCTTTTAATCGCGGCTCATCCCTCACACATCATCAAAGGATTCAT | | |
| | ACTGGGAGAAACCCTACCATTGTAACAGATGTGGGAAGACCTTTTATGACTGCACAGA | | |
| | CTTCAGTCAACATCCAGGAACTTTTATTAGGGAAAGAGTTTTTGAATATCACCACTGA | | |
| | AGAAAATCTGTGGTGAAAGGGAACATCTTACCATCTGGCCATTCACACTGAAGAGAAA | | |
| | CTTCATAAGCATCCTCTCTTTGAGAAAAC | | |
| | ORF Start: ATG at 7 | | ORF Stop: TGA at 1696 |
| | SEQ ID NO:330 | 563 aa | MW at 64300.6 kD |

| | |
|---|---|
| NOV117a, | MLETCGLLMSLGCPLFKPELIYQLDHRQELWMATKDLSQSSYPGDNTKPKTTEPTFSH |
| CG59807-01 Protein Sequence | |
| | LALPEEVLLQEQLTQGASKNSQLGQSKDQDGPSEMQEVHLKIGIGPQRGKLLEKMSSE |
| | RDGLGSDDGVCTKITQKQVSTEGDLYECDSHGPVTDALIREEKNSYKCEECQKVFKKN |
| | ALLVQHERTHTQVKPYECTECGKTFSKSTHLLQHLITHTGEKPYKCMECGKAFNRRSH |
| | LTRHQRTHSGEKPYKCSECGKAFTHRSTFVLHHRSHTGEKPFVCKECGKAFRDRPGFI |
| | RHYIIHTGEKPYECIECGKAFNRRSYLTWHQQIHTGVKPFECNECGKAFCESADLIQH |
| | YIIHTGEKPYKCMECGKAFNRRSHLKQHQRIHTGEKPYECSECGKAFTHCSTFVLHKR |
| | THTGEKPYECKECGKAFSDRADLIRHFSIHTGEKPYECVECGKAFNRSSHLTRHQQIH |
| | TGEKPYECIQCGKAFCRSANLIRHSIIHTGEKPYECSECGKAFNRGSSLTHHQRIHTG |
| | RNPTIVTDVGRPFMTAQTSVNIQELLLGKEFLNITTEENLW |

Further analysis of the NOV117a protein yielded the following properties shown in Table 117B.

TABLE 117B
Protein Sequence Properties NOV117a

| | |
|---|---|
| Psort | 0.4500 probability located in cytoplasm; 0.3000 probability |
| analysis: | located in microbody (peroxisome); 0.1000 probability |
| | located in mitochondrial matrix space; 0.1000 probability |
| | located in lysosome (lumen) |
| SignalP | Likely cleavage site between residues 19 and 20 |
| analysis: | |

A search of the NOV117a protein against the Geneseq database, a proprietary database that contains sequences published in patents and patent publication, yielded several homologous proteins shown in Table 117C.

TABLE 117C
Geneseq Results for NOV117a

| | | NOV117a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAM79549 | Human protein SEQ ID NO 3195 - | 1 . . . 563 | 563/566 (99%) | 0.0 |
| | Homo sapiens, 603 aa. | 38 . . . 603 | 563/566 (99%) | |
| | [WO200157190-A2, 9 AUG 2001] | | | |
| AAM78565 | Human protein SEQ ID NO 1227 - | 1 . . . 563 | 563/566 (99%) | 0.0 |
| | Homo sapiens, 603 aa. | 38 . . . 603 | 563/566 (99%) | |
| | [WO200157190-A2, 9 AUG | | | |
| | 2001] | | | |
| ABB21767 | Protein #3766 encoded by probe for | 44 . . . 562 | 375/519 (72%) | 0.0 |
| | measuring heart cell gene | 10 . . . 527 | 437/519 (83%) | |
| | expression - Homo sapiens, 551 aa. | | | |
| | [WO200157274-A2, 9 AUG | | | |
| | 2001] | | | |
| AAM69575 | Human bone marrow expressed | 44 . . . 562 | 375/519 (72%) | 0.0 |
| | probe encoded protein SEQ ID NO: | 10 . . . 527 | 437/519 (83%) | |
| | 29881 - Homo sapiens, 551 aa. | | | |
| | [WO200157276-A2, 9 AUG 2001] | | | |
| AAM57172 | Human brain expressed single exon | 44 . . . 562 | 375/519 (72%) | 0.0 |
| | probe encoded protein SEQ ID NO: | 10 . . . 527 | 437/519 (83%) | |
| | 29277 - Homo sapiens, 551 aa. | | | |
| | [WO200157275-A2, 9 AUG 2001] | | | |

In a BLAST search of public sequence databases, the NOV117a protein was found to have homology to the proteins shown in the BLASTP data in Table 117D.

TABLE 117D
Public BLASTP Results for NOV117a

| | | NOV117a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| O43296 | Zinc finger | 1 . . . 562 | 401/562 (71%) | 0.0 |
| | protein 264 - Homo | 43 . . . 603 | 468/562 (82%) | |
| | sapiens (Human), | | | |
| | 627 aa. | | | |
| Q96NL3 | CDNA FLJ30663 | 1 . . . 535 | 299/535 (55%) | 0.0 |
| | FIS, CLONE | 38 . . . 572 | 369/535 (68%) | |
| | SIMILAR TO ZINC | | | |
| | FINGER PROTEIN | | | |
| | 84 - Homo sapiens | | | |
| | (Human), 588 aa. | | | |
| Q99676 | Zinc finger | 2 . . . 535 | 261/542 (48%) | e−151 |
| | protein 184 - Homo | 58 . . . 595 | 355/542 (65%) | |
| | sapiens (Human), | | | |
| | 751 aa. | | | |
| Q96SE7 | ZINC FINGER | 151 . . . 541 | 233/391 (59%) | e−148 |
| | 1111 - Homo | 306 . . . 694 | 281/391 (71%) | |
| | sapiens (Human), | | | |
| | 839 aa. | | | |
| Q03923 | Zinc finger protein | 1 . . . 535 | 266/544 (48%) | e−148 |
| | 85 (Zinc finger | 33 . . . 547 | 328/544 (59%) | |
| | protein HPF4) | | | |
| | (HTF1) - Homo | | | |
| | sapiens (Human), | | | |
| | 595 aa. | | | |

PFam analysis predicts that the NOV117a protein contains the domains shown in the Table 117E.

TABLE 117E
Domain Analysis of NOV117a

| | | Identities/ | |
|---|---|---|---|
| | NOV117a | Similarities | |
| | Match | for the Matched | Expect |
| Pfam Domain | Region | Region | Value |
| KRAB: domain 1 of 1 | 1 . . . 34 | 14/66 (21%) | 0.15 |
| | | 24/66 (36%) | |
| zf-C2H2: domain 1 of 13 | 162 . . . 184 | 11/24 (46%) | 3.6e−06 |
| | | 19/24 (79%) | |
| zf-C2H2: domain 2 of 13 | 190 . . . 212 | 11/24 (46%) | 7.1e−06 |
| | | 19/24 (79%) | |
| zf-C2H2: domain 3 of 13 | 218 . . . 240 | 14/24 (58%) | 2.3e−07 |
| | | 22/24 (92%) | |
| zf-BED: domain 1 of 3 | 203 . . . 241 | 13/52 (25%) | 2 |
| | | 25/52 (48%) | |
| zf-C2H2: domain 4 of 13 | 246 . . . 268 | 11/24 (46%) | 4.6e−05 |
| | | 20/24 (83%) | |
| LIM: domain 1 of 1 | 220 . . . 284 | 16/72 (22%) | 0.69 |
| | | 50/72 (69%) | |
| zf-C2H2: domain 5 of 13 | 274 . . . 296 | 8/24 (33%) | 7.6e−05 |
| | | 18/24 (75%) | |
| zf-C2H2: domain 6 of 13 | 302 . . . 324 | 11/24 (46%) | 8.4e−05 |
| | | 20/24 (83%) | |
| Zn_carbOpept: domain 1 | 312 . . . 330 | 5/19 (26%) | 1.2 |
| of 1 | | 17/19 (89%) | |
| zf-C2H2: domain 7 of 13 | 330 . . . 352 | 8/24 (33%) | 9.7e−05 |
| | | 19/24 (79%) | |
| zf-C2H2: domain 8 of 13 | 358 . . . 380 | 14/24 (58%) | 5.3e−07 |
| | | 22/24 (92%) | |
| zf-BED: domain 2 of 3 | 343 . . . 381 | 12/52 (23%) | 1.3 |
| | | 26/52 (50%) | |
| zf-C2H2: domain 9 of 13 | 386 . . . 408 | 11/24 (46%) | 9.4e−05 |
| | | 20/24 (83%) | |
| zf-C2H2: domain 10 of 13 | 414 . . . 436 | 11/24 (46%) | 5e−06 |
| | | 20/24 (83%) | |
| zf-C2H2: domain 11 of 13 | 442 . . . 464 | 12/24 (50%) | 3e−07 |
| | | 22/24 (92%) | |
| zf-BED: domain 3 of 3 | 427 . . . 465 | 14/52 (27%) | 0.38 |
| | | 27/52 (52%) | |
| zf-C2H2: domain 12 of 13 | 470 . . . 492 | 12/24 (50%) | 0.00044 |
| | | 19/24 (79%) | |
| zf-C2H2: domain 13 of 13 | 498 . . . 520 | 12/24 (50%) | 9.8e−07 |
| | | 22/24 (92%) | |

### Example 118

The NOV118 clone was analyzed, and the nucleotide and predicted polypeptide sequences are shown in Table 118A.

TABLE 118A
NOV118 Sequence Analysis

| | SEQ ID NO:331 | 1899 bp | |
|---|---|---|---|
| NOV118a, | CAAACTCTACTACCTCTATATGACATTTCAGGTGTCTGTGACCTTTGATGATGTGGCT | | |
| CG59805-01 DNA Sequence | | | |
| | GTGACTTTCACCCAGGAGGAGTGGGGCCAGCTGGACCTAGCTCAGCGGACCCTGTACC | | |
| | AGGAGGTGATGCTGGAAAACTGTGGGCTCCTGGTATCTCTGGGTGGGTGTCCTGTTCC | | |
| | CAGACCTGAGCTGATCTACCACCTAGAGCATGGGCAGGAGCCATGGACCAGGAAGGAA | | |
| | GACCTCTCCCAAGGCACCTGTCCAGGTGACAAAGGAAAACCCAAGAGCACAGAACCTA | | |
| | CCACCTGTGAGCTAGCCTTGTCTGAAGGAATCTCTTTTTGGGGACAACTAACACAAGG | | |
| | AGCTTCAGGGGACTCCCAGTTGGGGCAACCCAAGGATCAGGATGGGTTTTCAGAAATG | | |
| | CAGGGAGAACGCTTGAGACCAGGGTTAGATTCCCAAAAGGAGAAGCTTCCTGGAAAAA | | |
| | TGAGCCCCAAACATGATGGTTTAGGGACAGCTGATAGTGTGTGTTCAAGGATTATACA | | |
| | GGATCGAGTCTCCTTAGGAGATGATGTCCATGACTGTGACTCACATGGATCAGGTAAA | | |
| | AATCCAGTTATTCAGGAAGAGGAAAATATCTTTAAATGCAATGAATGTGAAAAAGTGT | | |
| | TTAACAAGAAACGCCTGCTTGCTCGGCATGAGAGGATTCACTCTGGAGTGAAGCCCTA | | |
| | TGAATGCACAGAGTGTCGAAAAACCTTTAGCAAGAGTACATACCTCCTGCAGCACCAC | | |
| | ATGGTCCACACTGGGGAGAAGCCCTATAAGTGCATGGAGTGTGGGAAGGCTTTTAATC | | |
| | GGAAGTCACACCTTACCCAGCACCAGCGGATTCACAGTGGAGAGAAGCCTTATAAGTG | | |
| | CAGTGAATGTGGAAAGGCCTTCACCCACCGCTCCACTTTTGTCTTGCATAACAGGAGC | | |
| | CACACTGGAGAAAAACCCTTTGTGTGCAAAGAGTGTGGCAAAGCCTTTCGAGATAGGC | | |
| | CAGGTTTCATTCGACACTACATCATCCACAGTGGTGAGAATCCCTACGAGTGCTTCGA | | |
| | ATGTGGCAAGGTCTTCAAACACAGATCATACCTCATGTGGCACCAGCAGACTCATACC | | |
| | GGGGAGAAGCCCTATGAGTGCAGTGAATGTGGGAAGGCCTTCTGTGAGAGCGCAGCGC | | |
| | TGATTCACCACTATGTCATCCACACTGGAGAGAAGCCCTTTGAGTGCCTCGAGTGTGG | | |
| | GAAGGCTTTCAACCACCGATCCTACCTCAAAAGGCACCAGCGGATTCACACTGGGGAG | | |
| | AAGCCATATGTGTGTAGTGAATGCGGAAAGGCCTTCACCCACTGCTCTACTTTCATCT | | |
| | TGCATAAAAGGGCCCACACTGGAGAAAAACCTTTCGAGTGCAAAGAGTGTGGGAAAGC | | |
| | CTTTAGCAATAGGGCAGACCTCATTCGCCACTTCAGCATCCACACTGGAGAGAAGCCC | | |
| | TATGAGTGCATGGAGTGTGGAAAGGCCTTCAACCGCAGGTCAGGCCTCACAAGGCACC | | |
| | AGCGGATTCATAGTGGAGAGAAGCCCTATGAATGCATCGAGTGTGGGAAAACATTTTG | | |
| | CTGGAGCACAAACCTCATTCGACACTCTATCATCCACACTGGAGAGAAGCCGTATGAG | | |
| | TGCAGTGAATGTGGAAAGGCCTTCAGTCGCAGCTCGTCCCTCACTCAGCATCAAAGGA | | |
| | TGCATACTGGGAGAAATCCTATCAGTGTAACAGATGTGGGAAGACCTTTTACAAGTGG | | |
| | GCAGACCTCAGTCAACATCCAAGAACTTTTATTGGGGAAAAACTTTTTGAATGTCACC | | |
| | ACTGAGGAAAATCTTTTGCAAGAGGAAGCATCTTACATGGCATCTGATCGTACATACC | | |
| | AAAGAGAAACCCCACAAGTGTCTTCACTGTGAGAAAACCTTCT | | |
| | ORF Start: ATG at 20 | | ORF Stop: TGA at 1886 |
| | SEQ ID NO:332 | 622 aa | MW at 70677.2 kD |

| | |
|---|---|
| NOV118a, | MTFQVSVTFDDVAVTFTQEEWGQLDLAQRTLYQEVMLENCGLLVSLGGCPVPRPELIY |
| CG59805-01 Protein Sequence | |
| | HLEHGQEPWTRKEDLSQGTCPGDKGKPKSTEPTTCELALSEGISFWGQLTQGASGDSQ |
| | LGQPKDQDGFSEMQGERLRPGLDSQKEKLPGKMSPKHDGLGTADSVCSRIIQDRVSLG |
| | DDVHDCDSHGSGKNPVIQEEENIFKCNECEKVFNKKRLLARHERIHSGVKPYECTECG |
| | KTFSKSTYLLQHHMVHTGEKPYKCMECGKAFNRKSHLTQHQRIHSGEKPYKCSECGKA |
| | FTHRSTFVLHNRSHTGEKPFVCKECGKAFRDRPGFIRHYIIHSGENPYECFECGKVFK |
| | HRSYLMWHQQTHTGEKPYECSECGKAFCESAALIHHYVIHTGEKPFECLECCKAFNHR |
| | SYLKRHQRIHTGEKPYVCSECGKAFTHCSTFILHKRAHTGEKPFECKECGKAFSNRAD |
| | LIRHFSIHTGEKPYECMECGKAFNRRSGLTRHQRIHSGEKPYECIECGKTFCWSTNLI |
| | RHSIIHTGEKPYECSECGKAFSRSSSLTQHQRMHTGRNPISVTDVGRPFTSGQTSVNI |
| | QELLLGKNFLNVTTEENLLQEEASYMASDRTYQRETPQVSSL |

Further analysis of the NOV118a protein yielded the following properties shown in Table 118B.

TABLE 118B
Protein Sequence Properties NOV118a

| | |
|---|---|
| PSort | 0.4500 probability located in cytoplasm; 0.3796 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV118a protein against the Geneseq database, a proprietary database that contains sequences published in patents and patent publication, yielded several homologous proteins shown in Table 118C.

TABLE 118C
Geneseq Results for NOV118a

| | | | Identities/ | |
|---|---|---|---|---|
| | | NOV118a | Similarities | |
| | Protein/Organism/ | Residues/ | for | |
| Geneseq | Length | Match | the Matched | Expect |
| Number | [Patent #, Date] | Residues | Portion | Value |
| ABB22693 | Protein #4692 | 81 . . . 548 | 468/468 | 0.0 |
| | encoded by probe | 1 . . . 468 | (100%) | |
| | for measuring heart | | 468/468 | |
| | cell gene | | (100%) | |
| | expression - | | | |
| | Homo sapiens, | | | |
| | 468 aa. | | | |
| | [WO200157274-A2, | | | |
| | 09 AUG. 2001] | | | |
| AAM70526 | Human bone | 81 . . . 548 | 468/468 | 0.0 |
| | marrow expressed | 1 . . . 468 | (100%) | |
| | probe encoded | | 468/468 | |
| | protein SEQ | | (100%) | |
| | ID NO: | | | |
| | 30832 - Homo | | | |
| | sapiens, 468 aa. | | | |
| | [WO200157276-A2, | | | |
| | 09 AUG. 2001] | | | |
| AAM58080 | Human brain | 81 . . . 548 | 468/468 | 0.0 |
| | expressed single | 1 . . . 468 | (100%) | |
| | exon probe encoded | | 468/468 | |
| | protein SEQ ID NO: | | (100%) | |
| | 30185 - Homo | | | |
| | sapiens, 468 aa. | | | |
| | [WO200157275-A2, | | | |
| | 09 AUG. 2001] | | | |
| AAM30843 | Peptide #4880 | 81 . . . 548 | 468/468 | 0.0 |
| | encoded by probe | 1 . . . 468 | (100%) | |
| | for measuring | | 468/468 | |
| | placental gene | | (100%) | |
| | expression - Homo | | | |
| | sapiens, 468 aa. | | | |
| | [WO200157272-A2, | | | |
| | 09 AUG. 2001] | | | |
| AAM18364 | Peptide #4798 | 81 . . . 548 | 468/468 | 0.0 |
| | encoded by probe | 1 . . . 468 | (100%) | |
| | for measuring | | 468/468 | |
| | cervical gene | | (100%) | |
| | expression - Homo | | | |
| | sapiens, 468 aa. | | | |
| | [WO200157278-A2, | | | |
| | 09 AUG. 2001] | | | |

In a BLAST search of public sequence databases, the NOV118a protein was found to have homology to the proteins shown in the BLASTP data in Table 118D.

TABLE 118D
Public BLASTP Results for NOV118a

| | | NOV118a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| O43296 | Zinc finger | 4 . . . 622 | 530/619 (85%) | 0.0 |
| | sapiens | 11 . . . 627 | 567/619 (90%) | |
| | (Human), 627 aa. | | | |
| Q96NL3 | CDNA FLJ30663 | 7 . . . 572 | 334/566 (59%) | 0.0 |
| | FIS, CLONE | 9 . . . 573 | 403/566 (71%) | |
| | FCBBF1000598, | | | |
| | MODERATELY | | | |
| | SIMILAR TO | | | |
| | ZINC FINGER | | | |
| | PROTEIN 84 - | | | |
| | Homo sapiens | | | |
| | (Human), 588 aa. | | | |
| Q99676 | Zinc finger | 2 . . . 571 | 280/604 (46%) | e−160 |
| | protein 184 - Homo | 23 . . . 623 | 377/604 (62%) | |
| | sapiens (Human), | | | |
| | 751 aa. | | | |
| P51523 | Zinc finger | 4 . . . 617 | 286/637 (44%) | e−157 |
| | protein 84 (Zinc | 5 . . . 626 | 368/637 (56%) | |
| | finger protein | | | |
| | HPF2) - Homo | | | |
| | sapiens (Human), | | | |
| | 738 aa. | | | |
| Q9BX82 | EZFIT-RELATED | 7 . . . 617 | 278/621 (44%) | e−156 |
| | PROTEIN 1 - | 14 . . . 626 | 364/621 (57%) | |
| | Homo sapiens | | | |
| | (Human), 626 aa. | | | |

PFam analysis predicts that the NOV118a protein contains the domains shown in the Table 118E.

TABLE 118E
Domain Analysis of NOV118a

| | | Identities/ | |
|---|---|---|---|
| | NOV118a | Similarities | |
| | Match | for the Matched | Expect |
| Pfam Domain | Region | Region | Value |
| KRAB: domain 1 of 1 | 7 . . . 70 | 41/66 (62%) | 2.2e−33 |
| | | 54/66 (82%) | |
| zf-C2H2: domain 1 of 13 | 198 . . . 220 | 11/24 (46%) | 3.9e−05 |
| | | 17/24 (71%) | |
| BolA: domain 1 of 1 | 161 . . . 238 | 14/88 (16%) | 3.4 |
| | | 49/88 (56%) | |
| zf-C2H2: domain 2 of 13 | 226 . . . 248 | 10/24 (42%) | 6.2e−05 |
| | | 18/24 (75%) | |
| zf-C2H2: domain 3 of 13 | 254 . . . 276 | 14/24 (58%) | 5e−07 |
| | | 22/24 (92%) | |
| TFIIS: domain 1 of 1 | 257 . . . 292 | 12/39 (31%) | 5.7 |
| | | 21/39 (54%) | |
| zf-C2H2: domain 4 of 13 | 282 . . . 304 | 11/24 (46%) | 3.7e−05 |
| | | 20/24 (83%) | |
| LIM: domain 1 of 1 | 256 . . . 320 | 14/71 (20%) | 0.38 |
| | | 48/71 (68%) | |
| zf-C2H2: domain 5 of 13 | 310 . . . 332 | 8/24 (33%) | 7.6e−05 |
| | | 18/24 (75%) | |
| zf-C2H2: domain 6 of 13 | 338 . . . 360 | 11/24 (46%) | 1.1e−05 |
| | | 19/24 (79%) | |
| zf-C2H2: domain 7 of 13 | 366 . . . 388 | 9/24 (38%) | 0.00027 |
| | | 18/24 (75%) | |
| zf-C2H2: domain 8 of 13 | 394 . . . 416 | 12/24 (50%) | 7.9e−07 |
| | | 21/24 (88%) | |
| zf-C2H2: domain 9 of 13 | 422 . . . 444 | 10/24 (42%) | 0.00014 |
| | | 19/24 (79%) | |
| zf-C2H2: domain 10 of 13 | 450 . . . 472 | 10/24 (42%) | 8.3e−06 |
| | | 20/24 (83%) | |
| zf-C2H2: domain 11 of 13 | 478 . . . 500 | 13/24 (54%) | 3e−07 |
| | | 21/24 (88%) | |
| zf-BED: domain 1 of 1 | 463 . . . 501 | 14/52 (27%) | 0.1 |
| | | 29/52 (56%) | |
| zf-C2H2: domain 12 of 13 | 506 . . . 528 | 11/24 (46%) | 0.0016 |
| | | 17/24 (71%) | |
| zf-C2H2: domain 13 of 13 | 534 . . . 556 | 13/24 (54%) | 7.2e−08 |
| | | 23/24 (96%) | |

### Example 119

The NOV119 clone was analyzed, and the nucleotide and predicted polypeptide sequences are shown in Table 119A.

TABLE 119A
NOV119 Sequence Analysis

| | SEQ ID NO:333 | 1546 bp | |
|---|---|---|---|
| NOV119a, | GCTCAGTAGGCGTCGGGCTGTGATGCCCCAACTGCTCCAGCGTCTGCAGGCGCGCGCG | | |
| CG59928-01 DNA Sequence | | | |
| | GGCGCGGTAGGCGTACTCGCTGGCCGGATAGCGCGTGATGATGAACTGGTAGGTCTGC | | |
| | GCCGCATCGACGAACAGGCTCTCGCGCTCCAGGCATTGACCGCGCAGCAGGGAAATCT | | |
| | CCGGCTGCAGGTAATTGCGTGAGCGGCTCTTGCGCTCGGCCTGCGACAGCTCCAGCGC | | |
| | GACACGGGCGCAATCGCCTTCGTTGTAGGCGCGATAGGCGTTGTTCAGATGATGGTCG | | |
| | AGCGAGACACGGGTGCAACCCGCAGCAACCAGGGCCACGGCCAGAATGATCAGGTTAC | | |
| | GCATGGGCAATTCCTCCAATGAGCAGTGTATCGACAGCCCAGGCAAAAACTGAACAGC | | |
| | GGCAAGCCGACGACGGTTTTTCTGGCGGCGCCTTGGCATGACGCCACTGCCTCTCATT | | |
| | TTATCAACGCCAGCGCCACGACCGCTCGTCCTCTCGAACCAGCGCTAAATCCCCTTCT | | |
| | GCGCTGACCCATATCAATGCCGTTCAGCGCAACAGGGTGTGTAATGTAGGTACAGACT | | |
| | CCAGGCGAGGACGCTGCCATGAAACTGCAACGACTGTTGGTCGTCATCGACGCCGAAC | | |
| | ACCAGCAACAACCCGCCCTGCAACGCGCAGCCGATGTGGCACGCAAGACCGGCGCCGA | | |
| | ATTGCACCTGTTGCAGATCGAATACCACCCAAGCCTGGAAAGCGGCCTGCTGGACAGC | | |
| | CATCTGCTCAACCGCGCCCGTGAAACCATCCTGCGACAGAGCCACGAGGCCCTGCGTG | | |
| | CCAGCGTCGCTCACCTGAGCGATGAAGGATTCAAGATCGCAGTGGACGTGCGCTGGGG | | |
| | CAAACGTCGTCATGAAGAAATCCTCGCCCGCGTCGCGGTGTTGCAACCGGACATCCTG | | |
| | TTCAAGTCGACTCATCCCAGCAGTGCGCTGCGCCGCCTGTTGTTCAGTGATACCAGTT | | |
| | GGCAGCTGATTCGCCGCAGCCCGGTGCCGCTGTGGCTGGTACACGACGCCGAGCCCCA | | |
| | TGGTCAGAGCCTGTGCGCTGCGCTCGACCCGCTGCACAGCGCGGACAAACCTGCCGCC | | |
| | CTCGATCATCAGTTGATTGATGCCAGCCAGACCCTGCAGGCCGAGCTCGGCTTACAGG | | |
| | CCCAATACCTGCATGCACAGGCGCCTCTGCCGCGGTCGCTGCTGTTCGACGCCGAGGT | | |
| | AGCGCAGGAATATGAAGACTACGTGACCCAGTGCAGCCGCGAGCACCGCGAAGCCTTC | | |
| | GACAAGCTGATCGCCCAGCACGCCATCGATAGAGCACAGGCCCACCTGTTGGACGGTT | | |
| | TTGCCGAGGAAGTCATCCCGCGTTTCGTGCGTGAGCACAATATAGGCCTGCTGGTGAT | | |
| | GGGCGCCATCGCCCGCGGCCATCTGGACAGCCTGCTGATCGGCCACACCGCAGAACGG | | |
| | GTGCTGGAACGTGTCGAGTGCGATCTGCTGGTGATCAAATCGCACGGCAAAGGGTAGT | | |
| | GCACAGGAACAATGACTACAGCCCGACGCCTACTGAGC | | |
| | ORF Start: ATG at 599 | | ORF Stop: TAG at 1505 |
| | SEQ ID NO:334 | 302 aa | MW at 33922.3 kD |

| | |
|---|---|
| NOV119a, | MKLQRLLVVIDAEHQQQPALQRAADVARKTGAELHLLQIEYHPSLESGLLDSHLLNRA |
| CG59928-01 Protein Sequence | |
| | RETILRQSHEALRASVAHLSDEGFKIAVDVRWGKRRHEEILARVAVLQPDILFKSTHP |
| | SSALRRLLFSDTSWQLIRRSPVPLWLVHDAEPHGQSLCAALDPLHSADKPAALDHQLI |
| | DASQTLQAELGLQAQYLHAQAPLPRSLLFDAEVAQEYEDYVTQCSREEREAFDKLIAQ |
| | HAIDRAQAHLLDGFAEEVIPRFVREHNIGLLVMGAIARGHLDSLLIGHTAERVLERVE |
| | CDLLVIKSHGKG |

Further analysis of the NOV119a protein yielded the following properties shown in Table 119B.

TABLE 119B
Protein Sequence Properties NOV119a

| | |
|---|---|
| PSort | 0.3000 probability located in microbody (peroxisome); 0.3000 |
| analysis: | probability located in nucleus; 0.2014 probability located in |
| | lysosome (lumen); 0.1000 probability located in mitochondrial |
| | matrix space |
| SignalP | No Known Signal Sequence Predicted |
| analysis: | |

A search of the NOV119a protein against the Geneseq database, a proprietary database that contains sequences published in patents and patent publication, yielded several homologous proteins shown in Table 119C.

TABLE 119C
Geneseq Results for NOV119a

| | | | | |
|---|---|---|---|---|
| | | | Identities/ | |
| | | NOV119a | Similarities | |
| | Protein/Organism/ | Residues/ | for | |
| Geneseq | Length | Match | the Matched | Expect |
| Number | [Patent #, Date] | Residues | Portion | Value |

No Significant Matches Found

In a BLAST search of public sequence databases, the NOV119a protein was found to have homology to the proteins shown in the BLASTP data in Table 119D.

TABLE 119D
Public BLASTP Results for NOV119a

| | | NOV119a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9HW73 | HYPOTHETICAL | 1 . . . 297 | 156/299 (52%) | 1e−79 |
| | PROTEIN PA4328 - | 1 . . . 299 | 200/299 (66%) | |
| | Pseudomonas | | | |
| | aeruginosa, 304 aa. | | | |
| Q9KS28 | HYPOTHETICAL | 5 . . . 300 | 78/302 (25%) | 4e−29 |
| | PROTEIN | 6 . . . 304 | 147/302 (47%) | |
| | VC1433 - | | | |
| | Vibrio cholerae, | | | |
| | 315 aa. | | | |
| CAC91106 | PUTATIVE | 2 . . . 300 | 93/310 (30%) | 2e−28 |
| | STRESS | 3 . . . 303 | 137/310 (44%) | |
| | PROTEIN - | | | |
| | Yersinia pestis, | | | |
| | 318 aa. | | | |
| AAL20579 | PUTATIVE | 4 . . . 297 | 91/305 (29%) | 2e−28 |
| | UNIVERSAL | 5 . . . 300 | 139/305 (44%) | |
| | STRESS | | | |
| | PROTEIN - | | | |
| | Salmonella | | | |
| | typhimurium LT2, | | | |
| | 315 aa. | | | |
| CAD01669 | CONSERVED | 4 . . . 297 | 91/305 (29%) | 3e−28 |
| | HYPOTHETICAL | 5 . . . 300 | 139/305 (44%) | |
| | PROTEIN - | | | |
| | Salmonella enterica | | | |
| | subsp. enterica | | | |
| | serovar Typhi, | | | |
| | 315 aa. | | | |

PFam analysis predicts that the NOV119a protein contains the domains shown in the Table 119E.

TABLE 119E
Domain Analysis of NOVL19a

| | | Identities/ | |
|---|---|---|---|
| | NOV119a | Similarities | Expect |
| Pfam Domain | Match Region | for the Matched Region | Value |
| Usp: domain 1 of 2 | 2 . . . 144 | 28/153 (18%) | 0.0014 |
| | | 92/153 (60%) | |
| Usp: domain 2 of 2 | 160 . . . 297 | 28/153 (18%) | 0.013 |
| | | 88/153 (58%) | |

### Example 120

The NOV120 clone was analyzed, and the nucleotide and predicted polypeptide sequences are shown in Table 120A.

TABLE 120A
NOV120 Sequence Analysis

| | SEQ ID NO:335 | 2202 bp | |
|---|---|---|---|
| NOV120a, | CACCCTCCCGCCCCGCCCCCCGTCCAATGCTGAGCTCAGTCTGCGTCTCGTCCTTCCG | | |
| CG59947-01 DNA Sequence | | | |
| | CGGGCGCCAGGGGGCCAGCAAGCAGCAGCCGGCGCCACCGCCGCAGCCGCCCGAGGTC | | |
| | CCCGGTGGCGACAGCGGCAAGATCGTGATCAACGTGGGCGGCGTGCGCCATGAGACGT | | |
| | ACCGCTCGACGCTGCGCACCCTGCCGGGGACGCGGCTGGCCGGCCTGACGGAGCCCGA | | |
| | GGCGGCGGCACGCTTCGACTACGACCCGGGCGCCGACGAGTTCTTCTTTGACCGGCAC | | |
| | CCGGGAGTCTTCGCGTACGTGCTCAACTACTACCGCACCGGCAAGCTGCACTGCCCAG | | |
| | CCGACGTGTGCGGGCCCCTGTTTGAGGAGGAGCTCGGCTTCTGGGGCATCGACGAGAC | | |
| | CGACGTGGAGGCCTGCTGCTGGATGACCTACCGGCAGCATCGCGACGCTGAGGAGGCG | | |
| | CTCGACTCCTTCGAGGCGCCCGACCCCGCGGGCGCCGCCAACGCCGCCAACGCCGCAG | | |
| | GCGCCCACGACGGAGGCCTGGACGACGAGGCGGGCGCGGGCGGCGGCGGCCTGGACGG | | |
| | AGCGGGCGGCGAGCTCAAGCGCCTCTGCTTCCAGGACGCGGGCGGCGGCGCCGGGGGG | | |
| | CCGCCAGGGGGCGCGGGCGGCGCGGGCGGCACATGGTGGCGCCGCTGGCAGCCCCGCG | | |
| | TGTGGGCGCTCTTCGAGGACCCCTACTCGTCGCGGGCTGCCAGGTATGTGGCCTTCGC | | |
| | CTCCCTCTTCTTCATCCTCATCTCCATCACCACCTTCTGCCTGGAAACCCATGAGGGC | | |
| | TTCATCCATATTAGCAACAAGACGGTGACCCAGGCCTCCCCGATCCCCGGGGCACCTC | | |
| | CGGAGAACATCACCAACGTGGAGGTGGAGACGGAGCCCTTCCTGACCTACGTGGAGGG | | |
| | GGTGTGCGTGGTCTGGTTCACCTTCGAGTTCCTCATGCGCATCACCTTCTGCCCAGAC | | |
| | AAGGTGGAGTTTCTTAAAAGCAGCCTCAACATCATCGACTGTGTGGCCATCCTGCCCT | | |
| | TCTATCTCGAGGTGGGCCTCTCGGGCCTCAGCTCCAAGGCCGCCAAAGACGTGCTGGG | | |
| | CTTCCTGCGGGTGGTCCGCTTCGTCCGCATCCTGCGCATCTTCAAGCTGACCCGGCAC | | |
| | TTCGTGGGGCTGCGCGTGCTGGGACACACGCTCCGCGCCAGCACCAACGAGTTCCTGC | | |
| | TGCTCATCATCTTCCTGGCCCTGGGGGTGCTCATCTTCGCCACCATGATTTACTACGC | | |
| | TGAGCGCATTGGCGCCGACCCCGATGACATCCTGGGCTCCAACCACACCTACTTCAAG | | |
| | AACATCCCCATTGGCTTCTGGTGGGCTGTGGTCACCATGACGACCCTGGGCTATGGAG | | |
| | ACATGTACCCCAAGACGTGGTCGGGGATGCTGGTCGGGGCGCTGTGTGCCCTGGCGGG | | |
| | GGTGCTGACCATCGCCATGCCTGTGCCCGTCATTGTCAACAACTTTGGCATGTACTAT | | |
| | TCGCTGGCCATGGCCAAGCAGAAGCTGCCCAAGAAGAAGAACAAACACATCCCCCGGC | | |
| | CCCCGCAACCGGGCTCGCCCAACTACTGCAAGCCTGACCCACCCCCGCCACCCCCGCC | | |
| | CCACCCGCACCACGGCAGCGGGGGCATCAGCCCGCCGCCACCCATCACCCCACCCTCC | | |
| | ATGGGGGTGACTGTGGCCGGGGCCTACCCAGCGGGGCCCCACACGCACCCCGGGCTGC | | |
| | TCAGGGGGGGAGCGGGTGGGCTGGGGATCATGGGGCTGCCTCCTCTGCCAGCCCCCGG | | |
| | CGAGCCTTGCCCGTTGGCTCAGGAGGAGGTGATTGAGATCAACCGGGCAGATCCTCGC | | |
| | CCCAATGGGGATCCGGCAGCAGCTGCGCTTGCCCACGAGGACTGCCCAGCCATTGACC | | |
| | AGCCTGCCATGTCCCCGGAAGACAAGAGCCCCATCACGCCTGGAAGCCGTGGCCGCTA | | |
| | TAGCCGGGACCGAGCCTGCTTCCTCCTCACCGACTATGCCCCTTCCCCTGATGGCTCC | | |
| | ATCCGAAAAGCCACTGGTGCTCCCCCACTGCCCCCCCAAGACTGGCGTAAGCCAGGCC | | |
| | CCCCAAGCTTCTTGCCCGACCTCAACGCCAACGCCGCGGCCTGGATATCCCCCTAGTG | | |
| | GACGAACCCCCTCCCCCCGGGCTCTTGTCACCGCCTGAGACCTCGCGAGACTTTCG | | |
| | ORF Start: ATG at 27 | | ORF Stop: TAG at 2142 |
| | SEQ ID NO:336 | 705 aa | MW at 75590.5 kD |

| | |
|---|---|
| NOV120a, | MLSSVCVSSFRGRQGASKQQPAPPPQPPEVPGGDSGKIVINVGGVRHETYRSTLRTLP |
| CG59947-01 Protein Sequence | |
| | GTRLAGLTEPEAAARFDYDPGADEFFFDRHPGVFAYVLNYYRTGKLHCPADVCGPLFE |
| | EELGFWGIDETDVEACCWMTYRQHRDAEEALDSFEAPDPAGAANAANAAGAHDGGLDD |
| | EAGAGGGGLDGAGGELKRLCFQDAGGGAGGPPGGAGGAGGTWWRRWQPRVWALFEDPY |
| | SSRAARYVAFASLFFILISITTFCLETHEGFIHISNKTVTQASPIPGAPPENITNVEV |
| | ETEPFLTYVEGVCVVWFTFEFLMRITFCPDKVEFLKSSLNIIDCVAILPFYLEVGLSG |
| | LSSKAAKDVLGFLRVVRFVRILRIFKLTRHFVGLRVLGHTLRASTNEFLLLIIFLALG |
| | VLIFATMIYYAERIGADPDDILGSNHTYFKNIPIGFWWAVVTMTTLGYGDMYPKTWSG |
| | MLVGALCALAGVLTIAMPVPVIVNNFGMYYSLAMAKQKLPKKKNKHIPRPPQPGSPNY |
| | CKPDPPPPPPPHPHHGSGGISPPPPITPPSMGVTVAGAYPAGPHTHPGLLRGGAGGLG |
| | IMGLPPLPAPGEPCPLAQEEVIEINRADPRPNGDPAAAALAHEDCPAIDQPAMSPEDK |
| | SPITPGSRGRYSRDRACFLLTDYAPSPDGSIRKATGAPPLPPQDWRKPGPPSFLPDLN |
| | ANAAAWISP |

Further analysis of the NOV120a protein yielded the following properties shown in Table 120B.

TABLE 120B
Protein Sequence Properties NOV120a

| | |
|---|---|
| PSort | 0.6000 probability located in plasma membrane; 0.5071 |
| analysis: | probability located in mitochondrial inner membrane; |
| | 0.4000 probability located in Golgi body; 0.3000 probability |
| | located in endoplasmic reticulum (membrane) |
| SignalP | No Known Signal Sequence Predicted |
| analysis: | |

A search of the NOV120a protein against the Geneseq database, a proprietary database that contains sequences published in patents and patent publication, yielded several homologous proteins shown in Table 120C.

TABLE 120C
Geneseq Results for NOV120a

| | | | Identities/ | |
|---|---|---|---|---|
| | | NOV120a | Similarities | |
| | Protein/Organism/ | Residues/ | for | |
| Geneseq | Length | Match | the Matched | Expect |
| Number | [Patent #, Date] | Residues | Region | Value |
| AAY34120 | human potassium | 32 . . . 526 | 371/510 | 0.0 |
| | channel K + | 4 . . . 476 | (72%) | |
| | Hnov4 - Homo | | 399/510 | |
| | sapiens, 601 aa. | | (77%) | |
| | [WO9943696-A1, | | | |
| | 02 SEP. 1999] | | | |
| AAY32016 | Caenorhabditis | 33 . . . 512 | 217/486 | e−113 |
| | elegans cation | 27 . . . 465 | (44%) | |
| | channel protein - | | 300/486 | |
| | Caenorhabditis | | (61%) | |
| | elegans, 556 aa. | | | |
| | [WO9947923-A2, | | | |
| | 23 SEP. 1999] | | | |
| AAB86319 | Human Kv4.2 | 16 . . . 521 | 173/511 | 5e−69 |
| | protein - Homo | 22 . . . 441 | (33%) | |
| | sapiens, 629 aa. | | 256/511 | |
| | [DE19963612-A1, | | (49%) | |
| | 12 JUL. 2001] | | | |
| AAY13523 | Amino acid | 16 . . . 521 | 173/511 | 8e−68 |
| | sequence of | 23 . . . 442 | (33%) | |
| | KV4.2FL ion | | 257/511 | |
| | channel protein - | | (49%) | |
| | Mammalia, 630 aa. | | | |
| | [WO9923880-A1, | | | |
| | 20 MAY 1999] | | | |
| AAW42996 | Putative mature | 17 . . . 510 | 171/503 | 2e−66 |
| | potassium | 4 . . . 425 | (33%) | |
| | channel 2 | | 240/503 | |
| | protein - Homo | | (46%) | |
| | sapiens, 494 aa. | | | |
| | [U.S. Pat. | | | |
| | No. 5710019-A, | | | |
| | 20 JAN. 1998] | | | |

In a BLAST search of public sequence databases, the NOV120a protein was found to have homology to the proteins shown in the BLASTP data in Table 120D.

TABLE 120D
Public BLASTP Results for NOV120a

| | | NOV120a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q14003 | Voltage-gated | 1 . . . 705 | 704/757 (92%) | 0.0 |
| | potassium channel | 1 . . . 757 | 704/757 (92%) | |
| | protein Kv3.3 | | | |
| | (KSHIIID) - | | | |
| | Homo sapiens | | | |
| | (Human), 757 aa. | | | |
| Q01956 | Voltage-gated | 1 . . . 693 | 663/757 (87%) | 0.0 |
| | potassium channel | 1 . . . 756 | 668/757 (87%) | |
| | protein Kv3.3 | | | |
| | (KSHIIID) - Rattus | | | |
| | norvegicus (Rat), | | | |
| | 889 aa. | | | |
| Q63959 | Voltage−gated | 1 . . . 671 | 650/725 (89%) | 0.0 |
| | potassium channel | 1 . . . 724 | 653/725 (89%) | |
| | protein Kv3.3 | | | |
| | (KSHIIID) - Mus | | | |
| | musculus (Mouse), | | | |
| | 769 aa. | | | |
| A42073 | potassium channel | 32 . . . 607 | 557/576 (96%) | 0.0 |
| | protein Kv3.3 - | | | |
| | mouse, 679 aa. | | | |
| Q9PVD1 | KV3.1 | 34 . . . 671 | 441/640 (68%) | 0.0 |
| | POTASSIUM | 6 . . . 547 | 479/640 (73%) | |
| | CHANNEL - | | | |
| | Xenopus | | | |
| | laevis (African | | | |
| | clawed frog), | | | |
| | 592 aa. | | | |

PFam analysis predicts that the NOV120a protein contains the domains shown in the Table 120E.

TABLE 120E
Domain Analysis of NOV120a

| | | Identities/ | |
|---|---|---|---|
| | NOV120a | Similarities | |
| | Match | for the Matched | Expect |
| Pfam Domain | Region | Region | Value |
| K_tetra: domain 1 of 1 | 36 . . . 137 | 50/112 (45%) | 1.6e−47 |
| | | 86/112 (77%) | |
| thaumatin: domain 1 of 1 | 314 . . . 319 | 4/6 (67%) | 0.7 |
| | | 6/6 (100%) | |
| ion trans: domain 1 of 1 | 295 . . . 486 | 51/231 (22%) | 2.1e−29 |
| | | 155/231 (67%) | |

### Example 121

The NOV121 clone was analyzed, and the nucleotide and predicted polypeptide sequences are shown in Table 121A.

TABLE 121A
NOV121 Sequence Analysis

| | SEQ ID NO:337 | 1943 bp | |
|---|---|---|---|
| NOV121a, | AGATCCACGTGATCTCCAAAGACCCCTGTTGTCTTGTGTTGGGAGGTGGATCCTGAAT | | |
| CG59938-01 DNA Sequence | | | |
| | CCACCCAGAGAAGCCTGATACCAATAAAATCCCTGCTTGCTTTCCAGGAGACCCTTGG | | |
| | TCTTCATGTCTTTGGTGTGTGCACTCTTGAACACATGCCAGGCACACAGGGTGCATGA | | |
| | CGACAAGCCTAATATTGTCCTAATCATGGTTGATGACCTGGGTATTGGAGATCTGGGC | | |
| | TGCTACGGCAATGACACCATGAGGACGCCTCACATCGACCGCCTTGCCAGGGAAGGCG | | |
| | TGCGACTGACTCAGCACATCTCTGCCCCCTCCCTCTGCAGCCCAAGCCGGTCCGCGTT | | |
| | CTTGACGGGAAGATACCCCATCCGATCAGGTATGGTTTCTAGTGGTAATAGACGTGTC | | |
| | ATCCAAAATCTTGCAGTCCCCGCACGCCTCCCTCTTAATGAGACAACACTTGCAGCCT | | |
| | TGCTAAAGAAGCAAGGATACAGCACCGGGCTTATAGGTAACTTAGGCAAATGGCACCT | | |
| | GGGTTTGAGCTGCGCCTCTCGGAATGATCACTGTTACCACCCGCTCAACCATGGTTTT | | |
| | CACTACTTTTACGGGGTGCCTTTTGGACTTTTAAGCGACTGCCAGGCATCCAAGACAC | | |
| | CAGAACTGCACCGCTGGCTCAGGATCAAACTGTGGATCTCCACGGTAGCCCTTGCCCT | | |
| | GGTTCCTTTTCTGCTTCTCATTCCCAAGTTCGCCCGCTGGTTCTCAGTGCCATGGAAG | | |
| | GTCATCTTTGTCTTTGCTCTCCTCGCCTTTCTGTTTTTCACTTCCTGGTACTCTAGTT | | |
| | ATGGATTTACTCGACGTTGGAATTGCATCCTTATGAGGAACCATGAAATTATCCAGCA | | |
| | GCCAATGAAACACGAGAAAGTAGCTTCCCTCATGCTGAAGCAGGCACTTGCTTTCATT | | |
| | GAAAGGTACAAAAGGGAACCTTTTCTCCTCTTTTTTTCCTTCCTGCACGTACATACTC | | |
| | CACTCATCTCCAAAAAGAAGTTTGTTGGGCGCAGTAAATATGGCAGGTATGCCGACAA | | |
| | TGTAGAAGAAATGGATTGGATGGTGGGTGGTAAAATCCTGGATGCCCTGGACCAGGAG | | |
| | CGCCTGGCCAACCACACCTTGGTGTACTTCACCTCTGACAACGGGGGCCACCTGGAGC | | |
| | CCCTCCACGGGGCTGTTCAGCTGGGTGGCTGGAACGGGATCTACAAAGGTGGCAAAGG | | |
| | AATGGGAGGATGGGAAGGAGGTATCCGTCTGCCAGGGATATTCCGGTGGCCGTCAGTC | | |
| | TTGGACGCTGGGAGAGTGATCAATCAGCCCACCAGCTTAATCGACATCTATCCGACGC | | |
| | TGTCTTATATAGGCGGAGGGATCTTGTCCCAGGACAGAGTGATTGACGGCCAGAACCT | | |
| | AATGCCCCTGCTGGAACGAAGGGCGTCCCACTCCGACCACGAGTTCCTCTTCCACTAC | | |
| | TGTGGGGTCTATCTGCACACGGTCAGGTGGCATCAGAAGGACACTGTGTGGAAAGCTC | | |
| | ATTATGTGACTCCTAAATTCTACCCTGAAGGAACAGGTGCCTGCTATGGGAGTGGAAT | | |
| | ATGTTCATGTTCGGGCCATGTAACCTACCACCACCCACCACTCCTCTTTGACATCTCA | | |
| | AGAGACCCTTCAGAAGCCCTTCCACTGAACCCTGACAATGAGCCATTATTTGACTCCG | | |
| | TGATCAAAAAGATGGAGGCAGCCATAAGAGAGCATCGTAGGACACTAACACCTGTCCC | | |
| | ACAGCAGTTCTCTCTGTTCAACACAATTTGGAAACCATGGCTGCAGCCTTGCTCTGCG | | |
| | ACCTTCCCCTTCTGTGGGTGTGACAAGGAAGATGACATCCTTCCCATGGCTCCCTGAG | | |
| | ACCATGCGGACCACGTGTTACCCACCACAAACTTACTGTTACAATGGTCATAGGAGCA | | |
| | GAGCTCACCTGACTGATTCATTCCATTTG | | |
| | ORF Start: ATG at 122 | | ORF Stop: TGA at 1853 |
| | SEQ ID NO:338 | 577 aa | MW at 65099.5 kD |

| | |
|---|---|
| NOV121a, | MSLVCALLNTCQAHRVHDDKPNIVLTMVDDLGIGDLGCYGNDTMRTPHIDRLARECVR |
| CG59938-01 Protein Sequence | |
| | LTQHISAASLCSPSRSAFLTGRYFIRSGMVSSGNRRVIQNLAVPAGLPLNETTLAALL |
| | KKQGYSTGLIGKLGKWHLGLSCASRNDHCYHPLNHGFHYFYGVPFGLLSDCQASKTPE |
| | LHRWLRIKLWISTVALALVPFLLLIPKFARWFSVPWKVIEVFALLAFLFFTSWYSSYG |
| | FTRRWNCILMRNHEIIQQPMKEEKVASLMLKEALAFIERYKREPFLLFFSFLHVHTPL |
| | ISKKKFVGRSKYGRYGDNVEEMDWMVGGKILDALDQERLANHTLVYFTSDNGGHLEPL |
| | DGAVQLGGWNGIYKGGKGMGGWEGGIRVPGIFRWPSVLEAGRVINEPTSLMDIYPTLS |
| | YIGGGILSQDRVIDGQNLMPLLEGRASHSDHEFLFHYCGVYLHTVRWHQKDTVWKAHY |
| | VTPKFYPEGTGACYGSGICSCSGDVTYHDPPLLFDISRDPSEALPLNPDNEPLFDSVI |
| | KKMEAAIREHRRTLTPVPQQFSVFNTIWKPWLQPCCGTFPFCGCDKEDDILPMAP |

Further analysis of the NOV121a protein yielded the following properties shown in Table 121B.

TABLE 121B
Protein Sequence Properties NOV121a

| | |
|---|---|
| PSort | 0.6400 probability located in plasma membrane; 0.4600 |
| analysis: | probability located in Golgi body; 0.3700 probability located |
| | in endoplasmic reticulum (membrane); 0.1000 probability |
| | located in endoplasmic reticulum (lumen) |
| SignalP | No Known Signal Sequence Predicted |
| analysis: | |

A search of the NOV121a protein against the Geneseq database, a proprietary database that contains sequences published in patents and patent publication, yielded several homologous proteins shown in Table 121C.

TABLE 121C
Geneseq Results for NOV121a

| | | NOV121a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM78688 | Human protein SEQ ID NO 1350- | 1 . . . 572 | 388/576 (67%) | 0.0 |
| | Homo sapiens, 590 aa. | 10 . . . 580 | 449/576 (77%) | |
| | [WO200157190-A2, 9 AUG. 2001] | | | |
| AAM39343 | Human polypeptide SEQ ID NO | 20 . . . 571 | 331/555 (59%) | 0.0 |
| | 2488-Homo sapiens, 589 aa. | 37 . . . 587 | 404/555 (72%) | |
| | [WO200153312-A1, 26 JUL. 2001] | | | |
| AAM41129 | Human polypeptide SEQ ID NO | 20 . . . 571 | 331/555 (59%) | 0.0 |
| | 6060-Homo sapiens, 646 aa. | 94 . . . 644 | 404/555 (72%) | |
| | [WO200153312-A1, 26 JUL. 2001] | | | |
| AAY39920 | Human steroid sulphatase protein | 20 . . . 569 | 295/559 (52%) | e−166 |
| | sequence-Homo sapiens, 583 aa. | 26 . . . 575 | 374/559 (66%) | |
| | [WO9950453-A1, 7 OCT. 1999] | | | |
| AAB51185 | Human sulfatase protein C SEQ ID | 20 . . . 569 | 294/559 (52%) | e−165 |
| | NO: 14-Homo sapiens, 583 aa. | 26 . . . 575 | 372/559 (65%) | |
| | [U.S. Pat. No. 6153188-A, | | | |
| | 28 NOV. 2000] | | | |

In a BLAST search of public sequence databases, the NOV121a protein was found to have homology to the proteins shown in the BLASTP data in Table 121D.

TABLE 121D
Public BLASTP Results for NOV121a

| | | NOV121a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P54793 | Arylsulfatase F precursor (EC | 1 . . . 572 | 379/577 (65%) | 0.0 |
| | 3.1.6.-) (ASF)-Homo sapiens | 10 . . . 581 | 441/577 (75%) | |
| | (Human), 591 aa. | | | |
| AAH20229 | HYPOTHETICAL 64.9 KDA | 4 . . . 574 | 358/574 (62%) | 0.0 |
| | PROTEIN-Homo sapiens | 24 . . . 593 | 440/574 (76%) | |
| | (Human), 593 aa. | | | |
| P51689 | Arylsulfatase D precursor (EC | 4 . . . 574 | 349/574 (60%) | 0.0 |
| | 3.1.6.-) (ASD)-Homo sapiens | 24 . . . 593 | 429/574 (73%) | |
| | (Human), 593 aa. | | | |
| P51690 | Arylsulfatase E precursor (EC | 20 . . . 571 | 334/555 (60%) | 0.0 |
| | 3.1.6.-) (ASE)-Homo sapiens | 37 . . . 587 | 405/555 (72%) | |
| | (Human), 589 aa. | | | |
| P08842 | Steryl-sulfatase precursor (EC | 20 . . . 569 | 295/559 (52%) | e−166 |
| | 3.1.6.2) (Steroid sulfatase) (Steryl- | 26 . . . 575 | 374/559 (66%) | |
| | sulfate sulfohydrolase) | | | |
| | (Arylsulfatase C) (ASC)-Homo | | | |
| | sapiens (Human), 583 aa. | | | |

PFam analysis predicts that the NOV121a protein contains the domains shown in the Table 121E.

TABLE 121E
Domain Analysis of NOV121a

| | | Identities/ | |
|---|---|---|---|
| Pfam | NOV121a | Similarities for | Expect |
| Domain | Match Region | the Matched Region | Value |
| Sulfatase: | 21 . . . 504 | 231/530 (44%) | 1e−187 |
| domain 1 of 1 | | 410/530 (77%) | |

### Example 122

The NOV122 clone was analyzed, and the nucleotide and predicted polypeptide sequences are shown in Table 122A.

TABLE 122A
NOV122 Sequence Analysis

| | SEQ ID NO:339 | 3005 bp | |
|---|---|---|---|
| NOV122a, | ATTTCTTTGGTGTTGTCTTCACAGCTGAACTTGCAAAACAGATTGGAACTTCAAGATT | | |
| CG59746-01 DNA Sequence | | | |
| | ATCAATAATCGGAGATACGTATATTTTATTTGTAAAGAAAACATGGCTGCCCTATTCC | | |
| | TACGTGGTTTTGTCCAAATAGGGAACTGCAAGACTGGGATATCTAAGTCAAAAGAAGC | | |
| | ATTCATTGAAGCAGTGGAAAGAAAGAAGAAAGATAGACTGGTGCTGTATTTCAAAAGT | | |
| | GGAAAATATAGCACTTTTCCGCTAAGTGATAATATTCAAAATGTAGTCCTTAAATCCT | | |
| | ATAGAGGAAACCAAAATCACCTGCATTTAACTTTACAAAATAATAATGCCTTGTTTAT | | |
| | TGAAGGATTATCCTCCACAGATGCTGAACAATTGAAGATATTCTTGGACAGAGTTCAT | | |
| | CAAAACGAGGTTCAGCCACCTGTGAGACCTGGTAAGGGTGGGAGTGTCTTTTCTAGCA | | |
| | CAACACAGAAGGAAATCAACAAAACTTCATTCCAGAAAGTTGATGAGAAATCAAGTAG | | |
| | CAAATCTTTTGAGATAGCAAAAGGAAGTGGGACAGGTGTCCTTCAGAGGATGCCTTTG | | |
| | CTTACATCAAAATTGACACTTACTTGCGGAGAGTTATCAGAAAATCAGCACAAGAAGA | | |
| | GGAAAAGAATGCTCTCATCTAGCTCAGACATCAATGAGGAATTCTTGAAAGAAAATAA | | |
| | TTCTGTAGAATACAAGAAATCCAACGCAGATTGTTCGAGGTGTGTAAGCTATAATCGA | | |
| | GAGAAACAATTGAAGTTAAAAGAGTTAGAAGAGAATAAGAAATTGGAATGTGAATCTT | | |
| | CATGCATCATGAACGCCACTGGAAATCCTTACCTACATGACATTGGTCTTCTCCAAGC | | |
| | TCTCACTGAGAAAATGGTTTTGGTATTTCTGTTACAACAAGGGTATAGTGACGGTTAC | | |
| | ACAAAGTGGGATAAATTAAAACTATTTTTTGAATTATTTCCAGAGAAAATATGCCACG | | |
| | GCCTCCCCAATTTGGGAAACACCTGTTATATGAATGCAGTGTTACAGTCTCTACTTTC | | |
| | AATCCCATCGTTTGCTGATGATTTACTTAATCAGAGTTTCCCATGGGGTAAAATTCCC | | |
| | CTTAATGCTCTTACCATGTGCTTGGCACGGCTACTTTTTTTTAAACATACCTATAATA | | |
| | TAGAAATCAAGGAGATGTTACTCTTGAATCTTAAAAAGGCCATTTCAGCAGCTGCAGA | | |
| | GATATTCCATGGCAATGCACAGAACGATGCTCATGAGTTTTTAGCTCACTGTTTAGAT | | |
| | GGGAAGATAATTTTCCTAAACAGGTTTTTGCTGATGATCCTGACACCAGTGGGTTTTC | | |
| | TTGCCCTGTCATTACTAATTTTGAGTTAGAGTTGTTGCACTCCATTGCTTGTAAAGCT | | |
| | TGTGGTCAGGTTATTCTCAAGACAGAACTGAATAATTACCTCTCCATCAACCTTCCCC | | |
| | AAAGAATAAAACCACATCCTTCATCTATTCAGTCTACTTTTGATCTTTTTTTTCCAGC | | |
| | AGAAGAGCTTGAGTATAAATCTCCAAAATGTGAGCACAAGACTTCCGTTGCAGTGCAC | | |
| | TCATTCAGTAGGCTACCTACAATCCTTATTCTTCACCTCAAACGCTATACCTTGAATC | | |
| | AGTTTTGTGCATTAAACAAGAATGACCAGCAAGTCATCATTTCCAAATATTTAAACGT | | |
| | GTCTTCTCATTGCAATCAACGCACCAGACCACCTCTTCCCTTGAGTGAGGATGGAGAA | | |
| | ATTACAGATTTCCAATTATTAAAACTTATTCGAAAGATGACTTCTCCAAACATCACTG | | |
| | TATCATGGCCTGCAACAAAGGAATCCAAAGATATCCTGGCTCCACACATTCGATCAGA | | |
| | TAAGCAGTCTGAACAAAAAAAAGGCCAGACAGTCTTTAAAGGGGCAAGCAGAAGACAG | | |
| | CAGCAAAAGTACCTTGGAAAAAATTCTAAACCAAATGAGCTAGAATCTGTATACTCAG | | |
| | GAGATCGAGCATTCATTGAAAAACAACCGTTAGCTCACTTAATGACGTATCTGGAAGA | | |
| | TACCTCACTTTGTCAGTTCCACAAAGCTGGAGGTAAACCTCCCAGCAGCCCAGGCACA | | |
| | CCTCTCTCAAAAGTTCACTTTCAAACACTGCCCCAAAATCCAAAACCAAAGAAATATG | | |
| | TGAAAACCACTAAGTTTGTACCTTTTGATAGGATTATCAATCCTACTAAAGATTTGTA | | |
| | TGAAGATAAAAATATCAGAATTCCAGAAAGATTCCAAAAAGTGTCTGAACAGACTCAG | | |
| | CAGTGTGACGGTATGAGAATCTGTGAACAAGCCCCTCAGCAGGCACTGCCTCAAAGCT | | |
| | TTCCAAAGCCAGGCACCCACGCCCACACAAAGAACCTCCTAACACCTACAAAATTAAA | | |
| | TCTACAGAAGTCTAACAGGAATTCCCTACTTGCACTGGGTTCCAATAAGAATCCAAGA | | |
| | AACAAAGACATTTTAGATAAGATAAAATCTAAAGCCAAGGAAACAAAAAGAAATGATG | | |
| | ATAAGGGAGATCATACCTACCGGCTCATTAGTGTTGTCAGCCATCTTGGGAACACTCT | | |
| | AAACTCACCCCATTATATCTGTGATCCCTATCACTTTCACAAACACATCTCCTTCACT | | |
| | TACGATGATATCCCGGTGTTACCTATCCAGGAGGCCCAGATGCAGGAGGATAGGCCTT | | |
| | GCACTGGGTACATCTTCTTTTACATGCATAATGAGATCTTTGAAGAGATGTTGAAAAG | | |
| | AGAAGAGAATGCCCAGCTTAATAGCAAGGAGGTAGAGGAGACCCTTCAGAAGGAATAA | | |
| | GAGGAACGTACTCCTCCTTGTACAGATCTCCCTGACTGTCTCACTCGATACCACTTCC | | |
| | TCCATGGAAGGAAAACTGTGAACTTTATCCAGAGATGAAAATGCAATTAGTCTAGGAC | | |
| | CAAAGGTCAAACAGAAACACTTAATGGGGAGATCTGCATTCTAATCC | | |
| | ORF Start: ATG at 101 | | ORF Stop: TAA at 2840 |
| | SEQ ID NO:340 | 913 aa | MW at 104046.0 kD |

| | |
|---|---|
| NOV122a, | MAALFLRGFVQIGNCKTGISKSKEAFIEAVERKKKDRLVLYFKSGKYSTFRLSDNIQN |
| CG59746-01 Protein | |
| Sequence | VVLKSYRGNQNHLHLTLQNNNGLFIEGLSSTDAEQLKIFLDRVHQNEVQPPVRPGKGG |
| | SVFSSTTQKEINKTSFHKVDEKSSSKSFEIAKGSGTGVLQRMPLLTSKLTLTCGELSE |
| | NQHKKRKRMLSSSSEMNEEFLKENNSVEYKKSKADCSRCVSYNREKQLKLKELEENKK |
| | LECESSCIMNATGNPYLDDIGLLQALTEKMVLVFLLQQGYSDGYTKWDKLKLFFELFP |
| | EKICHGLPNLGNTCYMNAVLQSLLSIPSFADDLLNQSFPWGKIPLNALTMCLARLLFF |
| | KDTYNIEIKEMLLLNLKKAISAAAEIFHGNAQNDAHEFLAHCLDQLKDNMEKLNTIWK |
| | PKSEFGEDNFPKQVFADDPDTSGFSCPVITNFELELLHSIACKACGQVILKTELNNYL |
| | SINLPQRIKAHPSSIQSTFDLFFGAEELEYKCAKCEHKTSVGVHSFSRLPRILIVHLK |
| | RYSLNEFCALKKNDQEVIISKYLKVSSHCNEGTRPPLPLSEDGEITDFQLLKVIRKMT |
| | SGNISVSWPATKESKDILAPHIGSDKESEQKKGQTVFKGASRRQQQKYLGKNSKPNEL |
| | ESVYSGDRAFIEKEPLAHLMTYLEDTSLCQFHKAGGKPASSPGTPLSKVDFQTVPENP |
| | KRKKYVKTSKFVAFDRIINPTKDLYEDKNIRIPERFQKVSEQTQQCDGMRICEQAPQQ |
| | ALPQSFPKPGTQGHTKNLLRPTKLNLQKSNRNSLLALGSNKNPRNKDILDKIKSKAKE |
| | TKRNDDKGDHTYRLISVVSHLGKTLKSGHYICDAYDFEKQIWFTYDDMRVLGIQEAQM |
| | QEDRRCTGYIFFYMHNEIFEEMLKREENAQLNSKEVEETLQKE |

Further analysis of the NOV122a protein yielded the following properties shown in Table 122B.

TABLE 122B
Protein Sequence Properties NOV122a

| | |
|---|---|
| PSort | 0.7000 probability located in nucleus; 0.4270 probability |
| analysis: | located in mitochondrial matrix space; 0.3000 probability |
| | located in microbody (peroxisome); 0.1047 probability located |
| | in mitochondrial inner membrane |
| SignalP | Likely cleavage site between residues 16 and 17 |
| analysis: | |

A search of the NOV122a protein against the Geneseq database, a proprietary database that contains sequences published in patents and patent publication, yielded several homologous proteins shown in Table 122C.

TABLE 122C
Geneseq Results for NOV122a

| | | NOV122a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU07888 | Polypeptide sequence for human | 1 . . . 913 | 913/913 (100%) | 0.0 |
| | hspG25-Homo sapiens, 913 aa. | 1 . . . 913 | 913/913 (100%) | |
| | [WO200166752-A2, 13 SEP. 2001] | | | |
| AAB75607 | Human cancer associated antigen | 1 . . . 905 | 429/920 (46%) | 0.0 |
| | precursor HOM-TES-84/6 SEQ ID | 1 . . . 904 | 566/920 (60%) | |
| | NO: 6-Homo sapiens, 912 aa. | | | |
| | [WO200100874-A2, 4 JAN. 2001] | | | |
| AAU07869 | Polypeptide sequence for | 1 . . . 904 | 335/921 (36%) | e−147 |
| | mammalian Spg25-Mammalia, | 1 . . . 834 | 504/921 (54%) | |
| | 835 aa. [WO200166752-A2, | | | |
| | 13 SEP. 2001] | | | |
| AAG75460 | Human colon cancer antigen | 810 . . . 912 | 61/105 (58%) | 3e−28 |
| | protein SEQ ID NO: 6224-Homo | 3 . . . 107 | 79/105 (75%) | |
| | sapiens, 109 aa. [WO200122920- | | | |
| | A2, 5 APR. 2001] | | | |
| AAB39364 | Gene 8 human secreted protein | 810 . . . 871 | 39/64 (60%) | 5e−15 |
| | homologous amino acid sequence | 1 . . . 64 | 48/64 (74%) | |
| | #113-Bos taurus, 64 aa. | | | |
| | [WO200057903-A2, 5 OCT. 2000] | | | |

In a BLAST search of public sequence databases, the NOV122a protein was found to have homology to the proteins shown in the BLASTP data in Table 122D.

TABLE 122D
Public BLASTP Results for NOV122a

| | | NOV122a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9BXU7 | Ubquitin carboxyl-terminal | 1 . . . 913 | 913/913 (100%) | 0.0 |
| | hydrolase 26 (EC 3.1.2.15) | 1 . . . 913 | 913/913 (100%) | |
| | (Ubiquitin thiolesterase 26) | | | |
| | (Ubiquitin-specific processing | | | |
| | protease 26) (Deubiquitinating | | | |
| | enzyme 26)-Homo sapiens | | | |
| | (Human), 913 aa. | | | |
| Q9HBJ7 | UBIQUITIN-SPECIFIC | 1 . . . 905 | 429/920 (46%) | 0.0 |
| | PROCESSING PROTEASE-Homo | 1 . . . 904 | 566/920 (60%) | |
| | sapiens (Human), 922 aa. | | | |
| Q9HCH8 | KIAA1594 PROTEIN-Homo | 50 . . . 912 | 393/932 (42%) | e−171 |
| | sapiens (Human), 931 aa (fragment). | 3 . . . 929 | 535/932 (57%) | |
| Q99MX1 | Ubiquitin carboxyl-terminal | 1 . . . 904 | 335/921 (36%) | e−147 |
| | hydrolase 26 (EC 3.1.2.15) | 1 . . . 834 | 504/921 (54%) | |
| | (Ubiquitin thiolesterase 26) | | | |
| | (Ubiquitin-specific processing | | | |
| | protease 26) (Deubiquitinating | | | |
| | enzyme 26)-Mus musculus | | | |
| | (Mouse), 835 aa. | | | |
| Q9ES63 | UBIQUITIN-SPECIFIC | 1 . . . 908 | 341/933 (36%) | e−131 |
| | PROCESSING PROTEASE-Mus | 1 . . . 848 | 480/933 (50%) | |
| | musculus (Mouse), 869 aa. | | | |

PFam analysis predicts that the NOV122a protein contains the domains shown in the Table 122E.

TABLE 122E
Domain Analysis of NOV122a

| | | Identities/ | |
|---|---|---|---|
| Pfam | NOV122a | Similarities for | Expect |
| Domain | Match Region | the Matched Region | Value |
| UCH-1: | 295 . . . 326 | 21/32 (66%) | 8.8e−12 |
| domain 1 of 1 | | 29/32 (91%) | |
| UCH-2: | 820 . . . 885 | 20/72 (28%) | 2.2e−11 |
| domain 1 of 1 | | 47/72 (65%) | |

### Example 123

The NOV123 clone was analyzed, and the nucleotide and predicted polypeptide sequences are shown in Table 123A.

TABLE 123A
NOV123 Sequence Analysis

| | SEQ ID NO:341 | 2146 bp | |
|---|---|---|---|
| NOV123a, | GAAGGAGCGGGCATGAGGCGCTGCCCGTGCCGTGGGAGCCTGAACGAGGCGGAGGCCG | | |
| CG88613-01 DNA Sequence | | | |
| | GGGCGCTGCCCGCGGCGGCCCGCATGGGACTGGAGGCGCCGCGAGGAGGGCGCCGGCG | | |
| | GCAGCCGGGACAGCAGCGACCTGGGCCCGGCGCAGGGGCCCCGGCGGGGCGGCCGGAG | | |
| | GGGGGCGGGCCCTGGGCCCGGACACAGGGGTCCAGCCTCCACAGCGAGCCTGAGAGGG | | |
| | CCGGCCTCGGGCCTGCGCCGGGGACAGAGAGTCCGCAGGCAGAATTCTGGACAGACGG | | |
| | ACAGACTGAGCCCGCGGCAGCTGGCCTTGGAGTAGAGACCGAGAGGCCCAAGCAAAAG | | |
| | ACGGAGCCAGACAGGTCCAGCCTCCGGACGCATCTAGAATGGAGCTGGTCAGAGCTGG | | |
| | AGACGACTTGTCTTTGGACGGAGACCGGGACAGATGGCCTTTGGACTGATCCCCACAC | | |
| | GTCCGACCTCCAGTTTCAGCCCGAGGAGGCCAGCCCCTGGACACAGCCAGGGGTTCAT | | |
| | GGGCCCTGGACAGAGCTGGAAACGCATGCCTCACAGACTCAGCCAGAGAGGGTCAAGT | | |
| | CCTGGGCTGATAACCTCTGGACCCACCAGAACAGTTCCAGCCTCCAGACTCACCCAGA | | |
| | AGGAGCCTGTCCCTCAAAAGAGCCAAGTGCTGATGGCTCCTGGAAAGAATTGTATACT | | |
| | GATGGCTCCAGGACACAACAGGATATTGAAGGTCCCTGGACAGACCCATATACTGATG | | |
| | GCTCCCAGAAAAAACAGGATACTGAAGCAGCCAGGAAACAGCCTGGCACTGGTGGTTT | | |
| | CCAAATACAACAGGATACTGATGGCTCCTGGACACAACCTAGCACTGACGGTTCCCAG | | |
| | ACAGCACCTCGGACAGACTGCCTCTTCGGAGAGCCTCAGGATGGCCCATTAGAGGAAC | | |
| | CAGAGCCTGGAGAATTCCTCACTCACCTGTACTCTCACCTGAAGTGTAGCCCCCTGTG | | |
| | CCCTGTGCCCCGCCTCATCATTACCCCTGAGACCCCTCAGCCTGAGGCCCAGCCAGTG | | |
| | GGACCCCCCTCCCGGGTTGAGGGGGGCAGCGGCGGCTTCTCCTCTGCCTCTTCTTTCG | | |
| | ACGAGTCTGAGGATGACGTGGTCGCCGGGGGCGGAGGTGCCAGCGATCCCGAGGACAG | | |
| | GTCTGGGAGCAAACCCTGGAAGAAGCTGAAGACAGTTCTGAAGTATTCACCCTTTGTG | | |
| | GTCTCCTTCCGAAAACACTACCCTTGCGTCCAGCTTTCTGGACATGCTGGGAACTTCC | | |
| | AGGCAGGAGAGGATGGTCGCATTCTGAAACCTTTCTGTCAGTGTGACCAGCGCAGCCT | | |
| | GGAGCAGCTGATGAAAGACCCGCTGCGACCTTTCGTGCCTGCCTACTATGGCATGGTG | | |
| | CTGCAGGATGGCCAGACCTTCAACCAGATGGAAGACCTCCTGCCTGACTTTGAGGGCC | | |
| | CCTCCATTATGGACTGCAACATGGGCAGCAGCACCTATCTGGAAGAGGAGCTAGTGAA | | |
| | GGCACGGCAACGTCCCCGTCCCCGGAAGGACATCTATGAGAAGATGGTGGCTGTGGAC | | |
| | CCTGGGGCCCCTACCCCTGAGGAGCATGCCCAGCGTGCAGTCACCAAGCCCCGCTACA | | |
| | TGCAGTGGAGGGAAACCATGAGCTCCACCTCTACCCTGGGCTTCCGGATCGAGGGCAT | | |
| | CAAGAAGCCAGATGGGACCTGTAACACCAACTTCAAGAAGACGCAGGCACTGGAGCAG | | |
| | GTGACAAAAGTGCTGGAGGACTTCGTGGATGGAGACCACGTCATCCTGCAAAAGTACG | | |
| | TGGCATGCCTAGAAGAACTTCGTGAAGCTCTGCAGATCTCCCCCTTCTTCAAGACCCA | | |
| | CGAGGTGGTAGGCAGCTCCCTCCTCTTCGTGCACGACCACACCGGCCTGGCCAAGGTC | | |
| | TGGATGATAGACTTCGGCAAGACGGTGGCCTTGCCCGACCACCAGACGCTCAGCCACA | | |
| | GGCTGCCCTGGGCTGAGGGCAACCGTGAGGACGGCTACCTCTGGGGCCTGGACAACAT | | |
| | GATCTGCCTCCTGCAGGGGCTGGCACAGAGCTGAGCTGCTCAGCCACCATCAGGTTAA | | |
| | TTGGATGGCGCCAGTCTGGCTGGAGGAGCCCTGAGATGCCATGGGAGGCCTGAGGTTG | | |
| | ORF Start: ATG at 13 | | ORF Stop: TGA at 2062 |
| | SEQ ID NO:342 | 683 aa | MW at 75206.8 kD |

| | |
|---|---|
| NOV123a, | MRRCPCRGSLNEAEAGALPAAARMGLEAPRGGRRRQPGQQRPGPGAGAPAGRPEGGGP |
| CG88613-01 Protein Sequence | |
| | WARTEGSSLHSEPERAGLGPAPGTESPQAEFWTDGQTEPAAAGLGVETERPKQKTEPD |
| | RSSLRTHLEWSWSELETTCLWTETGTDGLWTDPHRSDLQFQPEEASPWTQPGVHGPWT |
| | ELETHGSQTQPERVKSWADNLWTHQNSSSLQTHPEGACPSKEPSADGSWKELYTDGSR |
| | TQQDIEGPWTEPYTDGSQKKQDTEAARKQPGTGGFQIQQDTDGSWTQPSTDGSQTAPG |
| | TDCLLGEPEDGPLEEPEPGELLTHLYSHLKCSPLCPVPRLIITPETPEPEAQPVGPPS |
| | RVEGGSGGFSSASSFDESEDDVVAGGGGASDPEDRSGSKPWKKLKTVLKYSPFVVSFR |
| | KHYPWVQLSGHAGNFQAGEDGRILKRFCQCEQRSLEQLMKDPLRPFVPAYYGMVLQDG |
| | QTFNQMEDLLADFEGPSIMDCKMGSRTYLEEELVKARERPRPRKDMYEKMVAVDPGAP |
| | TPEEHAQGAVTKPRYMQWRETMSSTSTLGFRIEGIKKADGTCNTNFKKTQALEQVTKV |
| | LEDFVDGDHVILQKYVACLEELREALEISPFFKTHEVVGSSLLFVHDHTGLAKVWMID |
| | FGKTVALPDHQTLSHRLPWAEGNREDGYLWGLDNMICLLQGLAQS |

Further analysis of the NOV123a protein yielded the following properties shown in Table 123B.

TABLE 123B
Protein Sequence Properties NOV123a

| | |
|---|---|
| PSort | 0.5663 probability located in microbody (peroxisome); 0.3000 |
| analysis: | probability located in nucleus; 0.1000 probability located in |
| | mitochondrial matrix space; 0.1000 probability located in |
| | lysosome (lumen) |
| SignalP | No Known Signal Sequence Predicted |
| analysis: | |

A search of the NOV123a protein against the Geneseq database, a proprietary database that contains sequences published in patents and patent publication, yielded several homologous proteins shown in Table 123C.

TABLE 123C
Geneseq Results for NOV123a

| | | NOV123a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM41393 | Human polypeptide SEQ ID NO | 1 . . . 683 | 682/683 (99%) | 0.0 |
| | 6324-Homo sapiens, 687 aa. | 5 . . . 687 | 682/683 (99%) | |
| | [WO200153312-A1, 26 JUL. 2001] | | | |
| AAM39607 | Human polypeptide SEQ ID NO | 12 . . . 683 | 642/680 (94%) | 0.0 |
| | 2752-Homo sapiens, 711 aa. | 36 . . . 711 | 643/680 (94%) | |
| | [WO200153312-A1, 26 JUL. 2001] | | | |
| AAE04364 | Human kinase (PKIN)-5-Homo | 273 . . . 682 | 219/432 (50%) | e−117 |
| | sapiens, 798 aa. [WO200146397- | 380 . . . 793 | 285/432 (65%) | |
| | A2, 28 JUN. 2001] | | | |

In a BLAST search of public sequence databases, the NOV123a protein was found to have homology to the proteins shown in the BLASTP data in Table 123D.

TABLE 123D
Public BLASTP Results for NOV123a

| | | NOV121a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96DU7 | INOSITOL 1,4,5- | 1 . . . 683 | 683/683 (100%) | 0.0 |
| | TRISPHOSPHATE 3-KINASE C- | 1 . . . 683 | 683/683 (100%) | |
| | Homo sapiens (Human), 683 aa. | | | |
| Q9Y475 | INOSITOL 1,4,5- | 83 . . . 683 | 601/601 (100%) | 0.0 |
| | TRISPHOSPHATE 3-KINASE | 4 . . . 604 | 601/601 (100%) | |
| | ISOENZYME (EC 2.7.1.127)- | | | |
| | Homo sapiens (Human), 604 aa | | | |
| | (fragment). | | | |
| S17682 | 1D-myo-inositol-trisphosphate 3- | 273 . . . 682 | 219/432 (50%) | e−117 |
| | kinase (EC 2.7.1.127) B-human, | 54 . . . 467 | 285/432 (65%) | |
| | 472 aa. | | | |
| CAB65055 | INOSITOL 1,4,5- | 273 . . . 682 | 219/432 (50%) | e−117 |
| | TRISPHOSPHATE 3-KINASE B- | 528 . . . 941 | 285/432 (65%) | |
| | Homo sapiens (Human), 946 aa. | | | |
| Q96JS1 | INOSITOL 1,4,5- | 273 . . . 682 | 219/432 (50%) | e−117 |
| | TRISPHOSPHATE 3-KINASE, | 528 . . . 941 | 285/432 (65%) | |
| | ISOFORM B (EC 2.7.1.127)- | | | |
| | Homo sapiens (Human), 946 aa. | | | |

PFam analysis predicts that the NOV123a protein contains the domains shown in the Table 123E.

TABLE 123E
Domain Analysis of NOV123a

| | | | |
|---|---|---|---|
| | | Identities/ | |
| Pfam | NOV123a | Similarities for | Expect |
| Domain | Match Region | the Matched Region | Value |

No Significant Matches Found

### Example 124

The NOV124 clone was analyzed, and the nucleotide and predicted polypeptide sequences are shown in Table 124A.

TABLE 124A
NOV124 Sequence Analysis

| | SEQ ID NO:343 | 1395 bp | |
|---|---|---|---|
| NOV124a, | GGTAAGACGACCTCTGGATGCTCACCCTGCCCTCTTCACCTCTCGTCCCCAGCTGTTT | | |
| CG59993-01 DNA Sequence | | | |
| | CCTCTGCCACCATGAGGAACATTTTCAAGAGGAACCAGGAGCCTATTGTGCCTCCTGC | | |
| | CACCACCACCGCCACGATGCCCATTGGACCCGTCGACAACTCCACTGAGAGTGCCGCT | | |
| | TAAACAAGATTCCCTTACCACCCTGGGCACTGATCGCCATTGCTCTGGTTGCTGGGCT | | |
| | CCTGCTTCTCACCTGCTGCTTCTGCATCTGCAAGAAATGCTGCTGCAAGAAGAAGAAG | | |
| | AACAAGAAGGAGAAGGCCAAAGGCATGAAGAATGCCATGAACATGAAGGACATGAAAG | | |
| | GGGGTCAGGATGACGACGACGCAGAGACACGCCTCACTGAGGGGGAAGGTGAAGGGGA | | |
| | GGAGGAGAAAGAGCCAGAGAACCTGGGCAAACTGCAGTTTTCCCTGGACTATGATTTT | | |
| | CAGGCTAATCAGCTTACTGTGGGCGTTCTGCAGGCTGCTGAACTGCCTGCCCTGGACA | | |
| | TGGGAGGCACCTCAGACCCTTATGTCAAGGTCTTCCTCCTTCCTGACAAGAAGAAGAA | | |
| | ATATGAGACCAAACTCCATCGGAAGACACTGAACCCTGCCTTCAATGAAACCTTCACC | | |
| | TTCAAGGTGCCATACCAGGAGCTTGGGGGCAAAACTCTGGTGATGGCCATCTATGACT | | |
| | TTGACCGCTTCTCCAAACATGACATCATTGCAGAGGTAAAGGTGCCTATCAACACAGT | | |
| | GGACCTCGGCCAGCCCATTGAGGAGTGGAGAGACCTGCAAGGCGGGGAAAAGCAGGAG | | |
| | CCGGAGAAGCTCGGCGACATCTGCACCTCCCTGCGCTATGTGCCCACGGCCGGGAAGC | | |
| | TCACTGTCTGCATCCTGGAGGCTAAGAACCTCAAGAAGATGGACGTGGGCGGCCTTTC | | |
| | AGACCCGTACGTGAAGATCCACCTGATGCAGAATGGCAAGAGGCTCAAGAAGAAGAAG | | |
| | ACAACCGTGAAGAAGAAGACCCTGAACCCATACTTCAACGAGTCCTTCAGCTTTGAGA | | |
| | TCCCCTTCGAGCAGATTCACAAAGTCCAGGTAGTCGTCACCGTGCTGGACTATGACAA | | |
| | GCTGCGCAAGAACGAAGCCATACGCAAGATCTTCGTGGGCAGCAATGCCACGGGCACA | | |
| | GAGCTGCGGCACTGGTCCGAGATGCTGGCCAACCCCCGGAGGCCCATCGCCCAGTGGC | | |
| | ACTCGCTCAAGCCTGAGGACGAGGTGCATGCACTCCTGGGCAACAACAAGTAGACAGC | | |
| | AGCGGCTGGGACCCCACACCTTTCACGGACACTGACAAGATCCAGAGCTATCAATACC | | |
| | TCA | | |
| | ORF Start: ATG at 70 | | ORF Stop: TAG at 1327 |
| | SEQ ID NO:344 | 419 aa | MW at 46871.8 kD |
| NOV124a, | MRNIFKRNQEPIVAPATTTATMPIGPVDNSTESGGAGESQEDMFAKLKEKLFNEINKI | | |
| CG59993-01 Protein Sequence | | | |
| | PLPPWALIAIAVVAGLLLLTCCFCICKKCCCKKKKNKKEKGKGMKNAMNMKDMKGGQD | | |
| | DDDAETGLTEGEGEGEEEKEPENLGKLQFSLDYDFQANQLTVGVLQAAELPALDMGGT | | |
| | SDPYVKVFLLPDKKKKYETKVHRKTLNPAFNETFTFKVPYQELGGKTLVMAIYDFDRF | | |
| | SKHDIIGEVKVPMNTVDLGQPIEEWRDLQGGEKEEPEKLGDICTSLRYVPTAGKLTVC | | |
| | ILEAKNLKKMDVGGLSDPYVKIHLMQNGKRLKKKKTTVKKKTLNPYFNESFSFEIPFE | | |
| | QIQKVQVVVTVLDYDKLGKNEAIGKIFVGSNATGTELRHWSDMLANPRRPIAQWHSLK | | |
| | PEEEVDALLGKNK | | |
| | SEQ ID NO:345 | 1338 bp | |
| NOV124b, | CCACCATGAGGAACATTTTCAAGACGAACCAGGAGCCTATTGTGGCTCCTCCCACCAC | | |
| CG59993-02 DNA Sequence | | | |
| | CACCGCCACGATGCCCATTGGACCCGTGGACAACTCCACTGAGAGTGGGGGTGCTGGG | | |
| | GAGAGTCAGGAGGACATCTTTGCCAAACTGAACGAGAAGTTATTCAATGAGATAAACA | | |
| | AGATTCCCTTACCACCCTGGGCACTGATCGCCATTGCTGTGGTTGCTGGGCTCCTGCT | | |
| | TCTCACCTGCTGCTTCTGCATCTGCAAGAAATGCTGCTGCAAGAAGAAGAAGAACAAG | | |
| | AAGGAGAAGGGCAAAGGTATGAAGAATGCCATGAACATGAAGGACATGAAAGGGGGTC | | |
| | AGGATGACGACGACGCAGAGACAGGCCTCACTGAGGGGGAAGGTGAAGGGGAGGAGGA | | |
| | AATCAGCTTACTGTGGGCCTTCTGCAGGCTGCTGAACTGCCTGCCCTGCACATGGGAG | | |
| | GCACCTCACACCCTTATGTCAAGGTCTTCCTCCTTCCTGACAAGAAGAAGAAATATGA | | |
| | GACCAAAGTCCATCGGAAGACACTGAACCCTGCCTTCAATGAAACCTTCACCTTCAAG | | |
| | GTCCCATACCAGGAGCTTGGGGGCAAAACTCTGGTCATGGCCATCTATGACTTTGACC | | |
| | GCTTCTCCAAACATGACATCATTGCAGAGGTAAAGGTGCCTATGAACACAGTGGACCT | | |
| | CGGCCAGCCCATTGAGGAGTGGAGAGACCTGCAAGGCGGCGAAAAGGAGGAGCCGGAG | | |
| | AAGCTGGGCGACATCTGCACCTCCCTGCGCTATGTGCCCACGGCCGGGAAGCTCACTG | | |
| | TCTGCATCCTGGAGGCTAAGAACCTCAAGAAGATGGACCTGGGCGGCCTTTCAGACCC | | |
| | ATGAACAAGAAGACCCTGAACCCATACTTCAACGAGTCCTTCAGCTTTGACATCCCCT | | |
| | TCCAGCAGATTCAGAAACTCCAGGTAGTGGTCACCGTGCTGGACTATGACAAGCTGGG | | |
| | CAAGAACGAAGCCATAGGCAAGATCTTCGTGGGCAGCAATGCCACGGGCACACAGCTG | | |
| | CGGCACTGGTCCGACATGCTGGCCAACCCCCGGAGGCCCATCGCCCAGTGGCACTCGC | | |
| | TCAAGCCTGAGGAGGAGGTGGGTGCACTCCTGGGCAAGAACAAGTAGACAGCAGCGGC | | |
| | TGGGACCCCACACCTTTCACGGACACTGACAAGATCCAGAGCTATCAATAAGGTGTAG | | |
| | GCGG | | |
| | ORF Start: ATG at 6 | | ORF Stop: TAG at 1263 |
| | SEQ ID NO:346 | 419 aa | MW at 46845.9 kD |

| | |
|---|---|
| NOV124b, | MRNIFKRNQEPIVAPATTTATMPIGPVDNSTESGGAGESQEDMFAKLKEKLFNEINKI |
| CG59993-02 Protein Sequence | |
| | PLPPWALIAIAVVAGLLLLTCCFCICKKCCCKKKKNKKEKGKGMKNAMNMKDMKGGQD |
| | DDDAETGLTEGEGEGEEEKEPENLGKLQFSLDYDFQANQLTVGVLQAAELPALDMGGT |
| | SDPYVKVFLLPDKKKKYETKVHRKTLNPAFNETFTFKVPYQELGGKTLVMAIYDFDRF |
| | SKHDIIGEVKVPMNTVDLGQPIEEWRDLQGGEKEEPEKLGDICTSLRYVPTAGKLTVC |
| | ILEAKNLKKMDVGGLSDPYVKIHLMQNGKRLKKKKTTVKKKTLNPYFNESFSFEIPFE |
| | QIQKVQVVVTVLDYDKLGKNEAIGKIFVGSNATGTELRHWSDMLANPRRPIAQWHSLK |
| | PEEEVDALLGKNK |

Sequence comparison of the above protein sequences yields the following sequence relationships shown in Table 124B.

TABLE 124B
Comparison of NOV124a against NOV124b.

| | | Identities/ |
|---|---|---|
| Protein | NOV124a Residues/ | Similarities for |
| Sequence | Match Residues | the Matched Region |
| NOV124b | 1 . . . 419 | 335/419 (79%) |
| | 1 . . . 419 | 335/419 (79%) |

Further analysis of the NOV124a protein yielded the following properties shown in Table 124C.

TABLE 124C
Protein Sequence Properties NOV124a

| | |
|---|---|
| PSort | 0.8202 probability located in mitochondrial inner |
| analysis: | membrane; 0.6000 probability located in endoplasmic |
| | reticulum (membrane); 0.3500 probability located in |
| | nucleus; 0.3034 probability located in mitochondrial |
| | intermembrane space |
| SignalP | No Known Signal Sequence Predicted |
| analysis: | |

A search of the NOV124a protein against the Geneseq database, a proprietary database that contains sequences published in patents and patent publication, yielded several homologous proteins shown in Table 124D.

TABLE 124D
Geneseq Results for NOV124a

| | | NOV124a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR97722 | Mouse inositol polyphosphate | 1 . . . 419 | 412/422 (97%) | 0.0 |
| | binding protein IP4-BP-Mus | 1 . . . 422 | 414/422 (97%) | |
| | musculus, 422 aa. [JP08092290-A, | | | |
| | 9 APR. 1996] | | | |
| AAU19715 | Human novel extracellular matrix | 128 . . . 405 | 141/280 (50%) | 2e−80 |
| | protein, Seq ID No 365-Homo | 169 . . . 447 | 201/280 (71%) | |
| | sapiens, 461 aa. [WO200155368- | | | |
| | A1, 2 AUG. 2001] | | | |
| AAU19714 | Human novel extracellular matrix | 141 . . . 409 | 140/273 (51%) | 3e−74 |
| | protein, Seq ID No 364-Homo | 11 . . . 281 | 193/273 (70%) | |
| | sapiens, 295 aa. [WO200155368- | | | |
| | A1, 2 AUG. 2001] | | | |
| AAW87702 | A human membrane fusion protein | 59 . . . 407 | 146/352 (41%) | 4e−73 |
| | designated SYTAX2-Homo | 31 . . . 364 | 220/352 (62%) | |
| | sapiens, 375 aa. [WO9856813-A2, | | | |
| | 17 DEC. 1998] | | | |
| AAO05534 | Human polypeptide SEQ ID NO | 33 . . . 164 | 127/135 (94%) | 5e−70 |
| | 19426-Homo sapiens, 149 aa. | 15 . . . 149 | 131/135 (96%) | |
| | [WO200164835-A2, 7 SEP. 2001] | | | |

In a BLAST search of public sequence databases, the NOV124a protein was found to have homology to the proteins shown in the BLASTP data in Table 124E.

TABLE 124E
Public BLASTP Results for NOV124a

| | | NOV124a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P29101 | Synaptotagmin II (SytII)-Rattus | 1 . . . 419 | 411/422 (97%) | 0.0 |
| | norvegicus (Rat), 422 aa. | 1 . . . 422 | 414/422 (97%) | |
| A55417 | synaptotagmin II-mouse, 422 aa. | 1 . . . 419 | 412/422 (97%) | 0.0 |
| | | 1 . . . 422 | 414/422 (97%) | |
| P46097 | Synaptotagmin II (SytII)-Mus | 1 . . . 419 | 411/422 (97%) | 0.0 |
| | musculus (Mouse), 422 aa. | 1 . . . 422 | 413/422 (97%) | |
| P24506 | Synaptotagmin B (Synaptic vesicle, | 10 . . . 419 | 341/413 (82%) | 0.0 |
| | protein O-P65-B)-Discopyge | 27 . . . 439 | 366/413 (88%) | |
| | ommata (Electric ray), 439 aa. | | | |
| P46096 | Synaptotagmin I (SytI) (p65)- | 10 . . . 419 | 323/418 (77%) | 0.0 |
| | Mus musculus (Mouse), 421 aa. | 8 . . . 421 | 353/418 (84%) | |

PFam analysis predicts that the NOV124a protein contains the domains shown in the Table 124F.

TABLE 124F
Domain Analysis of NOV124a

| | | Identities/ | |
|---|---|---|---|
| | | Similarities | |
| Pfam | NOV124a | for the | Expect |
| Domain | Match Region | Matched Region | Value |
| Adeno_E3_CR2: | 62 . . . 108 | 16/50 (32%) | 6.5 |
| domain 1 of 1 | | 26/50 (52%) | |
| C2: domain 1 of 2 | 156 . . . 242 | 54/97 (56%) | 1.8e−42 |
| | | 81/97 (84%) | |
| C2: domain 2 of 2 | 287 . . . 375 | 44/97 (45%) | 2.9e−39 |
| | | 80/97 (82%) | |

### Example 125

The NOV125 clone was analyzed, and the nucleotide and predicted polypeptide sequences are shown in Table 125A.

TABLE 125A
NOV125 Sequence Analysis

| | SEQ ID NO:347 | 3226 bp | |
|---|---|---|---|
| NOV125a, | GGACCACTTCTGATGCATCTCTGGGTCCCAACACTATCCACTGCAAGGCCTCGAAACA | | |
| CG59991-01 DNA Sequence | | | |
| | GGGGGGCCAGATGGCACCCCCATTTAGCACAAGAGACACGTCCACACTCTGTGAGCCC | | |
| | AAAGGGAGAAGGCTCAGGCCACGGCAGAGACGGAACCAGGAAAACGTCACGAAAAACA | | |
| | GCCTCAAGTTGCCAGGTCCCTTGCAGGAACAGACAGGCCTGGGGCCGCCCCACCTGGG | | |
| | CTCAGAGCTTGCGCTGCATCCAGGTGACACATGGGACTACAACAGTCACGTGATGACC | | |
| | AAATTCGCTGAGGAGGAGGATGTACGTCGTAGTTTTGAAAACACTGCTGCTGACTGGC | | |
| | CGGAAATGCAAACGTTCGCTGGTCCTTTTGATTCAGACCGGTGGGCCTTCCGGCCTCG | | |
| | CACGGTGGTTCTGCACGGAAAGTCAGGAATTGGGAAATCGGCTCTAGCCAGAAGGATC | | |
| | GTGCTGTGCTGGGCGCAAGGTGGACTCTACCAGGGAATGTTCTCCTACGTCTTCTTCC | | |
| | TCCCCGTTAGAGAGATGCAGCGGAAGAACGAGAGCAGTGTCACAGAGTTCATCTCCAG | | |
| | GGAGTGGCCAGACTCCCAGGCTCCGGTGACGGAGATCATGTCCCGACCAGAAAGGCTG | | |
| | TTGTTCATCATTGACGGTTTCGATGACCTGGGCTCTGTCCTCAACAATGACACAAAGC | | |
| | TCTGCAAAGACTGGGCTGAGAACCAGCCTCCGTTCACCCTCATACGCAGTCTGCTGAG | | |
| | GAAGGTCCTGCTCCCTGAGTCCTTCCTGATCGTCACCGTCAGAGACGTGGGCACAGAG | | |
| | CTTGCGTGCGATCATCAACAACCGTGAGCTCCTCCACCAGTGCCAGCTGCCCGCCGTC | | |
| | TGCCGTATGGCTGTGGAGGGAGTGTGGAATAGCAAGTCAGTGTTTGACGGTGACGACC | | |
| | TCATCGTTCAAGGACTCGCCGAGTCTGAGCTCCGTCCTCTCTTTCACATGAACATCCT | | |
| | TCTCCCAGACAGCCACTGTGAGGAGTACTACACCTTCTTCCACCTCAGTCTCCAGGAC | | |
| | TTCTGTGCCGCCTTGTACTACGTGTTAGAGGGCCTGGAAATCGAGCCAGCTCTCTGCC | | |
| | CTCTCTACGTTGAGAAGACAAAGAGGTCCATGGAGCTTAAACAGGCAGGCTTCCATAT | | |
| | CCACTCCCTTTGGATGAAGCCTTTCTTGTTTGGCCTCGTCACCGAAGACGTAAGGAGG | | |
| | CCACTGGAGGTCCTGCTGGGCTGTCCCGTTCCCCTGGGGGTGAAGCAGAAGCTTCTGC | | |
| | ACTGGGTCTCTCTGTTGGGTCAGCAGCCTAATGCCACCACCCCAGGAGACACCCTGGA | | |
| | CGCCTTCCACTGTCTTTTCCAGACTCAAGACAAAGAGTTTGTTCGCTTGGCATTAAAC | | |
| | AGCTTCCAACAACTGTGGCTTCCGATTAACCAGAACCTGGACTTGATAGCATCTTCCT | | |
| | TCTGCCTCCAGCACTGTCCGTATTTGCGGAAAATTCGGGTGGATGTCAAAGGCATCTT | | |
| | CCCAAGAGATGAGTCCCCTCAGGCATGTCCTCTGGTCCCTCTATGGATGCCGGATAAG | | |
| | ACCCTCATTGAGGAGCACTCCCAAGATTTCTGCTCCATGCTTGGCACCCACCCACACC | | |
| | TGCCAAGCTGAGGCATCCCACCTCCAACATACAGACCCTGATGTTTAGAAATGCACAG | | |
| | ATTACCCGTGCTGTCCAGCACCTCTGCAGAATCCTCATCGCCAACCGTAACCTAAGAT | | |
| | CCCTCAACTTCGGAGGCACCCACCTCAACGAAGAGGATGTAAGGATGCCCTGTGAACC | | |
| | CTTAAAACACCCAAAATGTTTGTTGGAGTCTTTGAGGCTGGATTCCTGTGGATTGACC | | |
| | CATGCCTGTTACCTGAACATCTCCCAAATCCTTACGACCTCCCCCAGCCTCAAATCTC | | |
| | TGAGCCTGGCACGAAACAAGGTGACACACCACGGAGTAATGCCTCTCAGTCATGCCTT | | |
| | GACACTCTCCCAGTGCGCCCTCCACAAGCTGATACTGGAGCACTCTGGCATCACACCC | | |
| | ACGGGTTGCCAGAGTCTCGCCTCAGCCCTCGTCAGCAACCGGAGCTTGACACACCTGT | | |
| | CCCTATCCAACAACACCCTGCCGAACGAAGGTGTAAATCTACTCTCTCGATCCATGAG | | |
| | GCTTCCCCACTGTAGTCTGCACAGGCTGATGCTGAATCAGTGCCACCTGGACACGGCT | | |
| | GGCTGTGCTTTTCTTGCACTTCCCCTTATGGGTAACTCATGCCTCACGCACCTGAGCC | | |
| | TTAGCATGAACCCTCTGGAAGACAATGGCCTCAACCTTCTGTGCCAGCTCATGAGAGA | | |
| | ACCATCTTGTCATCTCCACCACCTCCACTTGGTAAAGTCTCATCTCACCGCCCCGTGC | | |
| | CGGACAATGCCCTGGGTGACCCTCGCGTTCCTGCACTGTGCCAGCCACTGAAGCAAAA | | |
| | CAACACTGTTCTGACCACACTCGGGTTGAAGCCATGTCCACTGACTTCTCATTGCTGT | | |
| | GAGGCACTCTCCTTCCCCCTTTCCTCCAACCGGCATCTGACCAGTCTAAACCTGCTGC | | |
| | AGAATAACTTCACTCCCAAAGGAATGATCAAGCTCTGTTCGGCCTTTCCCTGTCCCAC | | |
| | GTCTAACTTACACATAATTCGCCTGTGGAAATGGCAGTACCCTGTCCAAATAACCAAG | | |
| | CTGCTCCACCAAGTGCAGCTACTCAACCCCCGAGTCGTAATTCACGGTAGTTCGCATT | | |
| | CTTTTCATCAACATGACCGGTACTGGTGGAAAAACTGAACATACCCAAACCTGCCCCA | | |
| | CTCACACCCATCTGATGGAGGAACTTTAAACCCTGT | | |
| | ORF Start: ATG at 69 | | ORF Stop: TGA at 3168 |
| | SEQ ID NO:348 | 1033 aa | MW at 116310.7 kD |

| | |
|---|---|
| NOV125a, | MGPPFSTRETSTLCEPKGRRLRPRQRRNQENVTKNSLKLPGPLQEQTGLGPPHLGSEL |
| CG59991-01 Protein | |
| Sequence | GLHGGDTWDYKSHVMTKFAEEEDVRRSFENTAADWPEMQTLAGAFDSDRWGFRPRTVV |
| | LHGKSGIGKSALARRIVLCWAQGGLYQGMFSYVFFLPVREMQRKKESSVTEFISREWP |
| | DSQAPVTEIMSRPERLLFIIDGFDDLGSVLNNDTKLCKDWAEKQPPFTLIRSLLRKVL |
| | LPESFLIVTVRDVGTEKLKSEVVSPRYLLVRGISGEQRIHLLLERGIGEHQKTQGLRA |
| | IMNNRELLDQCQVPAVGSLICVALQLQDVVGESVAPFNQTLTGLHAAFVFHQLTPRGV |
| | VRRCLNLEERVVLKRFCRMAVEGVWNRKSVFDGDDLMVQGLGESELRALFHMNILLPD |
| | SHCEEYYTFFHLSLQDFCAALYYVLEGLEIEPALCPLYVEKTKRSMELKQAGFHIHSL |
| | WMKRFLFGLVSEDVRRPLEVLLGCPVPLGVKQKLLHWVSLLGQQPNATTPGDTLDAFH |
| | CLFETQDKEFVRLALNSFQEVWLPINQNLDLIASSFCLQHCPYLRKIRVDVKGIFPRD |
| | ESAEACPVVPLWMRDKTLIEEQWEDFCSMLGTHPHLRQLDLGSSILTERAMKTLCAKL |
| | RHPTCKIQTLMFRNAQITPGVQHLWRIVMANRNLRSLNLGGTHLKEEDVRMACEALKH |
| | PKCLLESLRLDCCFLTHACYLKISQILTTSPSLKSLSLAGNKVTDQGVMPLSDALRVS |
| | QCALQKLILEDCGITATGCQSLASALVSNRSLTHLCLSNNSLGNEGVNLLCRSMRLPH |
| | CSLQRLMLNQCHLDTAGCGFLALALMGNSWLTHLSLSMNPVEDNGVKLLCEVMREPSC |
| | HLQDLELVKCHLTAACCESLSCVISRSRHLKSLDLTDNALGDGGVAALCEGLKQKNSV |
| | LTRLGLKACGLTSDCCEALSLALSCNRHLTSLNLVQNNFSPKGMMKLCSAFACPTSNL |
| | QIIGLWKWQYPVQIRKLLEEVQLLKPRVVIDGSWHSFDEDDRYWWKN |

Further analysis of the NOV125a protein yielded the following properties shown in Table 125B.

TABLE 125B
Protein Sequence Properties NOV125a

| | |
|---|---|
| PSort | 0.7600 probability located in nucleus; 0.3000 probability |
| analysis: | located in microbody (peroxisome); 0.1000 probability |
| | located in mitochondrial matrix space; 0.1000 probability |
| | located in lysosome (lumen) |
| SignalP | No Known Signal Sequence Predicted |
| analysis: | |

A search of the NOV125a protein against the Geneseq database, a proprietary database that contains sequences published in patents and patent publication, yielded several homologous proteins shown in Table 125C.

TABLE 125C
Geneseq Results for NOV125a

| | | NOV125a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAE07514 | Human PYRIN-1 protein - Homo | 103 . . . 934 | 276/843 (32%) | e−126 |
| | sapiens, 1034 aa. [WO200161005- | 207 . . . 1003 | 445/843 (52%) | |
| | A2, 23 AUG 2001] | | | |
| AAE07513 | Human nucleotide binding site 1 | 114 . . . 935 | 281/839 (33%) | e−120 |
| | (NBS-1) protein - Homo sapiens, | 180 . . . 990 | 431/839 (50%) | |
| | 1033 aa. [WO200161005-A2, | | | |
| | 23 AUG 2001] | | | |
| AAU07878 | Polypeptide sequence for | 207 . . . 963 | 218/766 (28%) | 7e−95 |
| | mammalian Spg65 - Mammalia, | 9 . . . 748 | 380/766 (49%) | |
| | 748 aa. [WO200166752-A2, | | | |
| | 13 SEP 2001] | | | |
| AAE06758 | Human G-protein coupled receptor- | 21 . . . 764 | 235/772 (30%) | 3e−88 |
| | 8 (GCREC-8) protein - Homo | 219 . . . 959 | 380/772 (48%) | |
| | sapiens, 1473 aa. [WO200157085- | | | |
| | A2, 9 AUG 2001] | | | |
| AAB62571 | Human CARD-7 polypeptide - | 21 . . . 764 | 235/772 (30%) | 3e−88 |
| | [WO200130813-A1, 3 MAY | 219 . . . 959 | 380/772 (48%) | |
| | 2001] | | | |

In a BLAST search of public sequence databases, the NOV125a protein was found to have homology to the proteins shown in the BLASTP data in Table 125D.

TABLE 125D
Public BLASTP Results for NOV125a

| | | | Identities/ | | |
|---|---|---|---|---|---|
| Protein | | NOV125a | Similarities | | |
| Accession | | Residues/ | for the | | Expect |
| Number | Protein/Organism/Length | Match Residues | Matched Portion | | Value |
| Q9JLR2 | MATERNAL-ANTIGEN-THAT- | 24 . . . 1033 | 548/1019 | (53%) | 0.0 |
| | EMBRYOS-REQUIRE PROTEIN - | 104 . . . 1111 | 716/1019 | (69%) | |
| | Mus musculus (Mouse), 1111 aa. | | | | |
| Q9R1M5 | MATER PROTEIN - Mus musculus | 24 . . . 1033 | 547/1019 | (53%) | 0.0 |
| | (Mouse), 1111 aa. | 104 . . . 1111 | 716/1019 | (69%) | |
| AAL35293 | NALP4 - Homo sapiens (Human), | 63 . . . 958 | 291/907 | (32%) | e−133 |
| | 994 aa. | 94 . . . 981 | 473/907 | (52%) | |
| Q96MN2 | CDNA FLJ32126 FIS, CLONE | 63 . . . 958 | 291/907 | (32%) | e−133 |
| | PEBLM2000112, WEAKLY | 19 . . . 906 | 473/907 | (52%) | |
| | SIMILAR TO HOMO SAPIENS | | | | |
| | NUCLEOTIDE-BINDING SITE | | | | |
| | PROTEIN 1 MRNA - Homo | | | | |
| | sapiens (Human), 919 aa. | | | | |
| AAL12497 | CRYOPYRIN - Homo sapiens | 103 . . . 934 | 276/843 | (32%) | e−125 |
| | (Human), 1034 aa. | 207 . . . 1003 | 445/843 | (52%) | |

PFam analysis predicts that the NOV125a protein contains the domains shown in the Table 125E.

TABLE 125E
Domain Analysis of NOV125a

| | | Identities/ | |
|---|---|---|---|
| | NOV125a | Similarities | |
| | Match | for the | |
| Pfam Domain | Region | Matched Region | Expect Value |
| LRR: domain 1 of 6 | 671 . . . 695 | 6/25 (24%) | 1.6e+02 |
| | | 16/25 (64%) | |
| LRR: domain 2 of 6 | 728 . . . 752 | 7/27 (26%) | 2.3e+02 |
| | | 17/27 (63%) | |
| LRR: domain 3 of 6 | 785 . . . 809 | 7/26 (27%) | 1.6e+02 |
| | | 19/26 (73%) | |
| LRR: domain 4 of 6 | 814 . . . 836 | 6/25 (24%) | 4.3e+02 |
| | | 14/25 (56%) | |
| LRR: domain 5 of 6 | 899 . . . 923 | 8/26 (31%) | 27 |
| | | 20/26 (77%) | |
| LRR: domain 6 of 6 | 956 . . . 977 | 7/25 (28%) | 2.9e+02 |
| | | 16/25 (64%) | |

The NOV126 clone was analyzed, and the nucleotide and predicted polypeptide sequences are shown in Table 126A.

TABLE 126A
NOV126 Sequence Analysis

| | SEQ ID NO:349 | 2310 bp | |
|---|---|---|---|
| NOV126a, | CCGCGCCTCAGTCCGCCGTCCGCCCTCCGCGCCCGCGCCGCTAGCATGACCGACGCGC | | |
| CG59987-01 DNA Sequence | | | |
| | TGTTGCCCGCGGCCCCCCAGCCGCTGGAGAAGGAGAACGACGGCTACTTTCGGAAGGG | | |
| | CTGTAATCCCCTTGCACAAACCGGCCGGAGTAAATTCCAGAATCAAAGAGCTGCTTTG | | |
| | AATCAGCAGATCCTGAAAGCCGTGCGGATGAGGACCGGAGCGGAAAACCTTCTGTTTG | | |
| | TGGCCACAAACTCAAAGGTGCGGGAGCAAGTGCGGCTGGAGCTGAGCTTCGTCAACTC | | |
| | AGACCTGCAGATGCTCAAGGAAGAGCTGGAGGGGCTGAACATCTCGGTGGGCGTCTAT | | |
| | CAGAACACAGAGGAGGCATTTACCATTCCCCTGATTCCTCTTGGCCTGAAGGAAACGA | | |
| | AAGACGTCGACTTTGCAGTCGTCCTCAAGGATTTTATCCTGGAACATTACAGTGAAGA | | |
| | TGGCTATTTATATCAAGATGAAATTGCACATCTTATGGATCTGACACAAGCTTGTCGG | | |
| | ACGCCTAGCCGGGATGAGGCCGGGGTGGAACTGCTGATGACATACTTCATCCAGCTGG | | |
| | GCTTTGTCGAGAGTCCATTCTTCCCCCCCACACGGCAGATGGGACTCCTGTTCACCTG | | |
| | GTATGACTCTCTCACCGGGGTTCCGGTCAGCCAGCAGAACCTGCTGCTGGAGAAGGCC | | |
| | AGTGTCCTGTTCAACACTGGGGCCCTCTACACCCAGATTGGGACCCGGTGTGATCGGC | | |
| | AGACGCAGGCTGGCCTGGAGAGTGCCATAGATGCCTTTCAGAGAGCCGCAGGGGTTTT | | |
| | AAATTACCTGAAAGACACATTTACCCATACTCCAAGTTACGACATGACCCCTGCCATG | | |
| | CTCAGCGTGCTCGTCAAAATGATGCTTCCACAAGCCCAAGAAAGCGTGTTTGAGAAAA | | |
| | TCAGCCTTCCTGCGATCCGGAATGAATTCTTCATGCTGGTGAAGGTGGCTCAGGAGGC | | |
| | AAAGAGAACATCCCCTACTCCTGGGCCAGCTTAGCCTGCGTGAAGGCCCACCACTACG | | |
| | CGGCCCTGCCCCACTACTTCACTGCCATCCTCCTCATCCACCACCAGGTGAAGCCAGG | | |
| | CACGGATCTGGACCACCAGGAGAAGTGCCTGTCCCAGCTCTACGACCACATGCCAGAG | | |
| | GGGCTGACACCCTTGGCCACACTGAAGAATGATCACCACCGCCGACAGCTGGGGAAGT | | |
| | CCCACTTGCGCAGAGCCATGGCTCATCACGAGGAGTCGGTGCCGGAGGCCAGCCTCTG | | |
| | CAAGAAGCTGCCGAGCATTGAGGTGCTACAGAAGGTGCTGTGTGCCGCACAGGAACGC | | |
| | TCCCGGCTCACGTACGCCCACCACCAGGAGGAGGATCACCTGCTGAACCTGATCGACC | | |
| | CCCCCAGAGTGTTGTTGCTAAAACTGAGCAAGAGGTTGACATTATATTGCCCCAGTTC | | |
| | TCCAGCTGACAGTCACGGACTTCTTCCAGAAGCTGGGCCCTTATCTGTGCTGTCGGCT | | |
| | AACAAGCGGTGGACGCCTCCTCGAAGCATCCGCTTCACTGCAGAACAAGGGGACTTGG | | |
| | CGTTCACCTTGAGAGGGAACGCCCCCGTTCACCTTCACTTCCTGGATCCTTACTGCTC | | |
| | TGCCTCGGTGGCAGCAGCCCGGGAAGGAGATTATATTGTCTCCATTCAGCTTGTGGAT | | |
| | TGTAAGTGGCTGACGCTGAGTGAGGTTATGAAGCTGCTGAAGAGCTTTGGCGAGGACG | | |
| | AGATCGAAATGAAAGTCGTGAGCCTCCTGGACTCCACATCATCCATGCATAATAAGAG | | |
| | TGCCACATACTCCGTGGGAATGCACAAAACGTACTCCATGATCTGCTTAGCCATTCAT | | |
| | GATGACGACAAAACTGATAAAACCAAGAAAATCTCCAAGAAGCTTTCCTTCCTGAGTT | | |
| | GGCTGCACGGCCTCACGTCAAGAAGAAGCTGCCCTCCCCTTTCAGCCTTCTCAACTCA | | |
| | GACACTTCTTGGTACTAATGTGAGGAAACAAACATGTTCAGGCCCCCAACATTTCCGG | | |
| | TGCTGACTCGGCCTTAAACGTTTGTGCCATAATGGAAAATATCTATCTATCTGTTCTC | | |
| | AAATCCTGTTTTTCTCATAGTGTAAACTCACATTTGATGTGTTTTTATGAAGGAAAGT | | |
| | AACCAAGAAACCTCTAGGAATTAGTGAAAAAAGAACTTTTTTGAGGTG | | |
| | ORF Start: ATG at 46 | | ORF Stop: TAA at 2104 |
| | SEQ ID NO:350 | 686 aa | MW at 76812.3 kD |
| NOV126a, | MTDALLPAAPQPLEKENDGYFRKGCNPLAQTGRSKLQNQRAALNQQILKAVRMRTGAE | | |
| CG59987-01 Protein Sequence | | | |
| | NLLKVATNSKVREQVRLELSFVNSDLQMLKEELEGLNISVGVYQNTEEAFTIPLIPLG | | |
| | LKETKDVDFAVVLKDFILEHYSEDGYLYEDEIADLMDLRQACRTPSRDEAGVELLMTY | | |
| | FIQLGFVESRFFPPTRQMGLLFTWYDSLTGVPVSQQNLLLEKASVLFNTGALYTQIGT | | |
| | RCDRQTQAGLESAIDAFQRAAGVLNYLKDTFTHTPSYDMSPAMLSVLVKMMLAQAQES | | |
| | VFEKISLPGIRNEFFMLVKVAQEAAKVGEVYQQLHAAMSQAPVKENIPYSWASLACVK | | |
| | AHHYAALAHYFTAILLIDHQVKPGTDLDHQEKCLSQLYDHMPEGLTPLATLKNDQQRR | | |
| | QLGKSELRRAMAHHEESVREASLCKKLRSIEVLQKVLCAAQERSRLTYAQHQEEDDLL | | |
| | NLIDAPRVLLLKLSKRLTLYCPSSPADSHGLLPEAGPLSVLSANKRWTPPRSIRFTAE | | |
| | EGDLGFTLRGNAPVQVHFLDPYCSASVAGAREGDYIVSIQLVDCKWLTLSEVMKLLKS | | |
| | FGEDEIEMKVVSLLDSTSSMHNKSATYSVGMQKTYSMICLAIDDDDKTDKTKKISKKL | | |
| | SFLSWGTNKNRQKSASTLCLPSVGAARPQVKKKLPSPFSLLNSDSSWY | | |
| | SEQ ID NO:351 | 2109 bp | |
| NOV126b, | CGCCGCTAGCATGACCGACGCGCTGTTGCCCGCGGCCCCCCAGCCGCTGGAGAAGGAG | | |
| CG59987-02 DNA Sequence | | | |
| | AACGACGGCTACTTTCGGAAGGGCTGTAATCCCCTTGCACAAACCGGCCGGAGTAAAT | | |
| | TGCAGAATCAAAGAGCTGCTTTGAATCAGCAGATCCTGAAAGCCGTGCGGATGAGGAC | | |
| | CGGAGCGGAAAACCTTCTGAAAGTGGCCACAAACTCAAAGGTGCGGGAGCAAGTGCGG | | |
| | CTGGAGCTGAGCTTCGTCAACTCAGACCTGCAGATCCTCAAGGAAGAGCTGGAGGGGC | | |
| | TGAACATCTCGCTGCGCGTCTATCACAACACAGACGAGGCATTTACGATTCCCCTGAT | | |
| | TCCTCTTGGCCTGAACGAAACCAAAGACGTCCACTTTGCACTCCTCCTCAACGATTTT | | |
| | ATCCTCGAACATTACAGTGAAGATGGCTATTTATATGAAGATGAAATTGCAGATCTTA | | |
| | TGGATCTGAGACAAGCTTGTCCGACGCCTAGCCGGGATGACGCCGGGGTGGAACTGCT | | |
| | GATGACATACTTCATCCAGCTGGGCTTTGTCCACAGTCGATTCTTCCCGCCCACACGG | | |
| | CAGATGGGACTCCTGTTCACCTGGTATGACTCTCTCACCGCGGTTCCGGTCACCCAGC | | |
| | AGAACCTGCTGCTGGAGAAGGCCAGTGTCCTGTTCAACACTGGGGCCCTCTACACCCA | | |
| | GATTCGGACCCGGTGCGATCGGCAGACGCAGGCTGCGCTGGAGACTGCCATAGATGCC | | |
| | TTTCAGAGAGCCGCACGGGTTTTAAATTACCTGAAAGACACATTTACCCATACTCCAA | | |
| | GTTACGACATGAGCCCTGCCATGCTCAGCGTGCTCGTCAAAATGATGCTTCCACAAGC | | |
| | CCAAGAAACCGTGTTTGAGAAAATCAGCCTTCCTCCGATCCCGAATGAATTCTTCATG | | |
| | CTGGTGAAGGTGGCTCAGGAGGCTGCTAAGGTGGGAGACGTCTACCAACAGCTACACC | | |
| | CTGCGTGAAGGCCCACCACTACGCGGCCCTGGCCCACTACTTCACTGCCATCCTCCTC | | |
| | CAGCCATCAGCCAGGCCCCCGTGAAAGACAACATCCCCTACTCCTGGGCCAGCTTAGC | | |
| | ATCGACCACCAGGTGAAGCCAGGCACGGATCTGGACCACCAGGACAAGTGCCTGTCCC | | |
| | GCAGCGCCGACAGCTCGGGAAGTCCCACTTGCGCACACCCATCGCTCATCACGAGCAG | | |
| | TCGGTGCGGGAGGCAAGCCTCTGCAAGAAGCTGCGGAGCATTGACGTGCTACAGAAGG | | |
| | TGCTGTGTGCCGCACAGGAACGCTCCCGGCTCACGTACGCCCAGCACCAGGAGGAGGA | | |
| | TGACCTGCTGAACCTGATCGACGCCCCCAGTGTTGTTGCTAAAACTGAGCAAGAGGTT | | |
| | GACATTATATTGCCCCAGTTCTCCAAGCTGACAGTCACGGACTTCTTCCAGAACCTCG | | |
| | GCCCCTTATCTGTGTTTTCGGCTAACAAGCGGTGGACGCCTCCTCGAAGCATCCGCTT | | |
| | CACTGCAGAAGAAGGGGACTTGGGGTTCACCTTGAGAGGGAACGCCCCCGTTCAGGTT | | |
| | CACTTCCTCGATCCTTACTCCTCTGCCTCCCTGCCAGGAGCCCGCGAAGGACATTATA | | |
| | TTGTCTCCATTCAGCTTCTGGATTGTAAGTCGCTGACGCTGAGTGAGGTTATGAAGCT | | |
| | GCTGAAGAGCTTTGGCGAGGACGAGATCGAGATCAAAGTCGIGAGCCTCCTGGACTCC | | |
| | ACATCATCCATGCATAATAAGAGTGCCACATACTCCCTGGGAATGTAGAAAACGTACT | | |
| | CCATGATCTCCTTAGCCATTCATGATGACGACAAAACTGATAAAACCAAGAAAATCTC | | |
| | CAAGAAGCTTTCCTTCCTGAGTTGGGGCACCAACAACAACAGACACAAGTCAGCCAGC | | |
| | ACCTTGTGCCTCCCATCGGTCGGGGCTGCACGGCCTCAGGTCAAGAAGAAGCTGCCCT | | |
| | CCCCTTTCACCCTTCTCAACTCACACACTTCTTGGTACTAATGTGAGCAAACAAACAT | | |
| | GTTCAGGCCCCGAACATTTCC | | |
| | ORF Start: ATG at 11 | | ORF Stop: TAG at 1844 |
| | SEQ ID NO:352 | 611 aa | MW at 68613.9 kD |

| | |
|---|---|
| NOV126b, | MTDALLPAAPQPLEKENDGYFRKGCNPLAQTGRSKLQNQRAALNQQILKAVRMRTGAE |
| CG59987-02 Protein Sequence | |
| | NLLKVATNSKVREQVRLELSFVNSDLQMLKEELEGLNISVGVYQNTEEAFTIPLIPLG |
| | LKETKDVDFAVVLKDFILEHYSEDGYLYEDEIADLMDLRQACRTPSRDEAGVELLMTY |
| | FIQLGFVESRFFPPTRQMGLLFTWYDSLTGVPVSQQNLLLEKASVLFNTGALYTQIGT |
| | RCDRQTQAGLESAIDAFQRAAGVLNYLKDTFTHTPSYDMSPAMLSVLVKMMLAQAQES |
| | VFEKISLPGIRNEFFMLVKVAQEAAKVGEVYQQLHAAMSQAPVKENIPYSWASLACVK |
| | AHHYAALAHYFTAILLIDHQVKPGTDLDHQEKCLSQLYDHMPEGLTPLATLKNDQQRR |
| | QLGKSELRRAMAHHEESVREASLCKKLRSIEVLQKVLCAAQERSRLTYAQHQEEDDLL |
| | NLIDAPRVLLLKLSKRLTLYCPSSPADSHGLLPEAGPLSVLSANKRWTPPRSIRFTAE |
| | EGDLGFTLRGNAPVQVHFLDPYCSASVAGAREGDYIVSIQLVDCKWLTLSEVMKLLKS |
| | FGEDEIEMKVVSLLDSTSSMHNKSATYSVGM |

Sequence comparison of the above protein sequences yields the following sequence relationships shown in Table 126B.

TABLE 126B
Comparison of NOV126a against NOV126b.

| | | Identities/ |
|---|---|---|
| | | Similarities |
| Protein | NOV126a Residues/ | for the |
| Sequence | Match Residues | Matched Region |
| NOV126b | 1 . . . 611 | 585/612 (95%) |
| | 1 . . . 611 | 590/612 (95%) |

Further analysis of the NOV126a protein yielded the following properties shown in Table 126C.

TABLE 126C
Protein Sequence Properties NOV126a

| | |
|---|---|
| PSort | 0.4500 probability located in cytoplasm; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV126a protein against the Geneseq database, a proprietary database that contains sequences published in patents and patent publication, yielded several homologous proteins shown in Table 126D.

TABLE 126D
Geneseq Results for NOV126a

| | | NOV126a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAU10192 | Human prostate specific protein | 1 . . . 686 | 660/687 | (96%) | 0.0 |
| | PSL22 - Homo sapiens, 686 aa. | 1 . . . 686 | 665/687 | (96%) | |
| | [WO200172962-A2, 4 OCT 2001] | | | | |
| AAB68561 | Human GTP-binding associated | 27 . . . 686 | 626/661 | (94%) | 0.0 |
| | protein #61 - Homo sapiens, 666 aa. | 7 . . . 666 | 633/661 | (95%) | |
| | [WO200105970-A2, 25 JAN 2001] | | | | |
| AAG64579 | Human transcription termination | 201 . . . 686 | 458/487 | (94%) | 0.0 |
| | factor binding protein 54 - Homo | 3 . . . 488 | 464/487 | (95%) | |
| | sapiens, 488 aa. [CN1297918-A, | | | | |
| | 6 JUN 2001] | | | | |
| AAB29661 | Human histidine domain-protein | 110 . . . 357 | 82/252 | (32%) | 3e−28 |
| | tyrosine phosphatase, SEQ ID NO:2 | 7 . . . 253 | 135/252 | (53%) | |
| | [WO200063392-A1, 26 OCT 2000] | | | | |
| AAU00869 | Human cancer related protein 5 - | 409 . . . 597 | 70/189 | (37%) | 2e−27 |
| | Homo sapiens, 257 aa. | 8 . . . 196 | 102/189 | (53%) | |
| | [WO200118014-A1, 15 MAR | | | | |
| | 2001] | | | | |

In a BLAST search of public sequence databases, the NOV126a protein was found to have homology to the proteins shown in the BLASTP data in Table 126E.

TABLE 126E
Public BLASTP Results for NOV126a

| | | NOV126a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q96RU1 | RHOPHILIN-LIKE PROTEIN - | 1 . . . 686 | 627/688 (91%) | 0.0 |
| | Homo sapiens (Human), 685 aa. | 1 . . . 685 | 640/688 (92%) | |
| Q9DBN2 | 1300002E07RIK PROTEIN - | 1 . . . 686 | 573/687 (83%) | 0.0 |
| | Mus musculus (Mouse), 686 aa. | 1 . . . 686 | 616/687 (89%) | |
| Q61085 | GTP-RHO binding protein 1 | 16 . . . 596 | 273/583 (46%) | e−135 |
| | (Rhophilin) - Mus musculus | 20 . . . 580 | 361/583 (61%) | |
| | (Mouse), 643 aa. | | | |
| Q9XYY9 | RHOPHILIN - Drosophila | 21 . . . 615 | 248/654 (37%) | e−110 |
| | melanogaster (Fruit fly), 718 aa. | 31 . . . 674 | 363/654 (54%) | |
| Q96PV9 | KIAA1929 PROTEIN - Homo | 23 . . . 366 | 178/346 (51%) | 1e−93 |
| | sapiens (Human), 410 aa | 17 . . . 362 | 241/346 (69%) | |
| | (fragment). | | | |

PFam analysis predicts that the NOV126a protein contains the domains shown in the Table 126F.

TABLE 126F
Domain Analysis of NOV126a

| | | Identities/ | | |
|---|---|---|---|---|
| | | Similarities | | |
| Pfam | NOV126a | for the | | Expect |
| Domain | Match Region | Matched Region | | Value |
| HR1: domain 1 of 1 | 38 . . . 110 | 19/87 | (22%) | 1.2e−05 |
| | | 53/87 | (61%) | |
| BRO1: domain 1 of 1 | 111 . . . 263 | 60/172 | (35%) | 3.8e−56 |
| | | 125/172 | (73%) | |
| PDZ: domain 1 of 1 | 516 . . . 593 | 20/84 | (24%) | 0.46 |
| | | 53/84 | (63%) | |

### Example 127

The NOV127 clone was analyzed, and the nucleotide and predicted polypeptide sequences are shown in Table 127A.

TABLE 127A
NOV127 Sequence Analysis

| | SEQ ID NO:353 | 3351 bp | |
|---|---|---|---|
| NOV127a, | CGTCCCGTGGCCATGACGACCGCTCAGAGGGACTCCCTGTTGTGGAAGCTCGCGGGGT | | |
| CG59971-01 DNA Sequence | | | |
| | TGCTGCGCGAGTCCCGTCATGTGGTCCTGTCTGGCTGTAGCACCCTGAGCCTGCTGAC | | |
| | TCCCACACTGCAACAGCTGAACCACGTATTTGAGCTGCACCTGGGGCCATGGGGCCCT | | |
| | GGCCAGACAGGCTTTGTGGCTCTGCCCTCCCATCCTGCCGACTCCCCTGTTATTCTTC | | |
| | AGCTTCAGTTTCTCTTCGATGTGCTGCAGAAAACACTTTCACTCAAGCTGGTCCATGT | | |
| | TGCTGGTCCTGGCCCCACAGGGCCCATCAAGATTTTCCCCTTCAAATCCCTTCGGCAC | | |
| | CTGGAGCTCCCAGGTGTTCCCCTCCACTGTCTCCATGGCCTCCCAGGCATCTACTCCC | | |
| | AGCTACAATGCACTGACCGCCTTAGACAGCTCCCTGCGCCTCTTGTCAGCTCTGCGTT | | |
| | TCTTGAACCTAAGCCACAATCAAGTCCAGGACTGTCAGGGATTCCTGATGGATTTGTG | | |
| | TGAGCTCCACCATCTGGACATCTCCTATAATCGCCTGCATTTGGTGCCAAGAATGGGA | | |
| | CCCTCAGGGGCTGCTCTGGGGGTCCTGATACTGCGAGGCAATGAGCTTCGGAGCCTGC | | |
| | CAGGCCTAGAGCAGCTGAGGAATCTGCGGCACCTGGATTTGGCATACAACCTGCTGGA | | |
| | AGGACACCGGGAGCTGTCACCACTGTGGCTGCTGGCTGAGCTCCGCAAGCTCTACCTG | | |
| | GAGGGGAACCCTCTTTGGTTCCACCCTGAGCACCGAGCAGCCACTGCCCAGTACTTGT | | |
| | CACCCCGGGCCACCCATGCTGCTACTGGCTTCCTTCTCGATGGCAAGGTCTTGTCACT | | |
| | GACAGATTTTCAGCAGACTCACACATCCTTGCGGCTCAGCCCCATGGGCCCACCTTTG | | |
| | CCCTGGCCAGTGGGGAGTACTCCTGAAACCTCAGGTCGCCCTGACCTGAGTGACAGCC | | |
| | TCTCCTCAGGGGGTGTTGTGACCCAGCCCCTGCTTCATAAGGTTAAGAGCCGAGTCCG | | |
| | TGTGAGGCGGGCAAGCATCTCTGAACCCAGTGATACGGACCCGGAGCCCCGAACTCTG | | |
| | AACCCCTCTCCGGCTGGTTGGTTCGTGCAGCAGCACCCGGAGCTGGAGCTCATGAGCA | | |
| | GCTTCCGGGAACGGTTCGGCCGCAACTGGCTGCAGTACAGGAGTCACCTGGAGCCCTC | | |
| | CGGAAACCCTCTGCCGGCCACCCCCACTACTTCTGCACCCAGTGCACCTCCAGCCAGC | | |
| | TCCCAGGGCCCCGACACTGCACCCAGACCTTCACCCCCGCAGCAGGAAGCCAGAGGCC | | |
| | CCCAGGAGTCACCACAGAAAATGTCAGAGGAGGTCAGGGCGGAGCCACAGGAGGAGGA | | |
| | AGAGGAGAAGGAGGGGAAGGAGGAGAAGGAGGAGGGGGAGATGGTGGAACAGGGAGAA | | |
| | GAGGAGGCAGGAGAGGAGGAAGAAGAGGAGCAGGACCAGAAGGAAGTGGAAGCGGAAC | | |
| | TCTGTCGCCCCTTGTTGGTGTGTCCCCTGGAGGGGCCTGAGGGCGTACGGGGCAGGGA | | |
| | ATGCTTTCTCAGGGTCACTTCTGCCCACCTGTTTGAGGTGGAACTCCAAGCAGCTCGC | | |
| | ACCTTGGAGCGACTGGAGCTCCAGAGTCTGGAGGCAGCTGAGATAGAGCCGGAGGCCC | | |
| | AGGCCCAGGGTCCCCCTCTTGCTCCGCAGGGCTCAGATCTGCTCCCTGGAGCCCCCAT | | |
| | CCTCAGTCTGCGCTTCTCCTACATCTGCCCTGACCGGCAGTTGCGTCGCTATTTGGTG | | |
| | CTGGAGCCTGATGCCCACGCAGCTGTCCACCAGCTGCTTGCCGTGTTGACCCCAGTCA | | |
| | CCAATGTGGCTCGGGAACAGCTTGGGGAGGCCAGGGACCTCCTGCTGGGTAGATTCCA | | |
| | GTCTCTACGCTGTGGCCATGAGTTCAAGCCAGAGGAGCCCAGCATCGGATTAGACAGT | | |
| | GAGGAAGGCTGGAGGCCTCTGTTCCAAAAGACAGAATCTCCTGCTGTGTGTCCTAACT | | |
| | AGTCGCATAGAGCTGGGCCTGGCAGCCCAGAGCCTGCGGCTAGAGTGGGCAGCTGGGG | | |
| | CGGGCCGCTGTGTGCTGCTGCCCCGACATGCCAGGCATTGCCGGGCCTTCCTAGAGCA | | |
| | GCTCCTTGGTGTCTTGCAGTCTCTCCCCCCTGCCTGGAGGAACTCTGTCAGTGCCACA | | |
| | GAGGAGGAGGTCACCCCCCAGCACCGGCTCTGGCCATTGCTGGAAAAAGACTCATCCT | | |
| | GCAGCTGATGCTTCCCTCCACCTGCCTCGTATCCCTGTTGCTGACTCCCTCCACCCTG | | |
| | TTCCTGTTAGATGAGGATGCTGCAGGGTCCCCGGCAGAGCCCTCTCCTCCAGCACCAT | | |
| | CTGCCGAAGCCTCTGAGAAGGTGCCTCCCTCGGCGCCGCGCCCTGCTGTCCGTGTCAG | | |
| | GGAGCAGCAGCCACTCAGCAGCCTGAGCTCCGTGCTGCTCTACCGCTCAGCCCCTGAG | | |
| | GACTTGCGGCTGCTCTTCTACCATGAGGTGTCCCGGCTGGAGAGCTTTTCGGCACTCC | | |
| | GGAGCTGTTTTCCATCCGACTCCGCACACTCATCCAAGAGGCGCTCGCCCTTCACCGA | | |
| | TGAGGGTCCCACGCTGACCTTGGCCCTGACCTCAGGAGCCACGCT | | |
| | ORF start: ATG at 13 | | ORF Stop: TGA at 3307 |
| | SEQ ID NO:354 | 1098 aa | MW at 121004.1 kD |
| NOV127a, | MTTAQRDSLLWKLAGLLRESGDVVLSGCSTLSLLTPTLQQLNHVFELHLGPWGPGQTG | | |
| CG59971-01 Protein | | | |
| Sequence | FVALPSHPADSPVILQLQFLFDVLQKTLSLKLVHVAGPGPTGPIKIFPFKSLRHLELR | | |
| | GVPLHCLHGLRGIYSQLETLICSRSLQALEELLSACGGDFCSALPWLALLSANFSYNA | | |
| | LTALDSSLRLLSALRFLNLSHNQVQDCQGFLMDLCELHHLDISYNRLHLVPRMGPSGA | | |
| | ALGVLILRGNELRSLPGLEQLRNLRHLDLAYNLLEGHRELSPLWLLAELRKLYLEGNP | | |
| | LWFHPEHRAATAQYLSPRARDAATGFLLDGKVLSLTDFQQTHTSLGLSPMGPPLPWPV | | |
| | GSTPETSGGPDLSDSLSSGGVVTQPLLHKVKSRVRVRRASISEPSDTDPEPRTLNPSP | | |
| | AGWFVQQHPELELMSSFRERFGRNWLQYRSHLEPSGNPLPATPTTSAPSAPPASSQGP | | |
| | DTAPRPSPPQEEARGPQESPQKMSEEVRAEPQEEEEEKEGKEEKEEGEMVEQGEEEAG | | |
| | EEEEEEQDQKEVEAELCRPLLVCPLEGPEGVRGRECFLRVTSAHLFEVELQAARTLER | | |
| | LELQSLEAAEIEPEAQAQGPPLATGSDLLPGAPILSLRFSYICPDRQLRRYLVLEPDA | | |
| | AHAAVQELLAVLTPVTNVAREQLGEARDLLLGRFQCLRCGHEFKPEEPRMGLDSEEGW | | |
| | RPLFQKTESPAVCPNCGSDHVVLLAVSRGTPNRERKQGEQSLAPSPSASPVCHPPGHG | | |
| | DHLDRAKNSPPQAPSTRDHGSWSLSPAPERCGLRSVDHRLRLFLDVEVFSDAQEEFQC | | |
| | CLKVPVALAGHTGEFMCLVVVSDRRLYLLKVTGEMSEPPASWLQLTLAVPLQDLSGIE | | |
| | LGLAGQSLRLEWAAGAGRCVLLPRDARHCRAFLEELLGVLQSLPPAWRNCVSATEEEV | | |
| | TPQHRLWPLLEKDSSLEARQFFYLRAFLVEGEASVQLMLPSTCLVSLLLTPSTLFLLD | | |
| | EDAAGSPAEPSPPAASGEASEKVPPSGPGPAVRVREQQPLSSLSSVLLYRSAPEDLRL | | |
| | LFYDEVSRLESFWALRVVCQEQLTALLAWIREPWEELFSIGLRTVIQEALALDR | | |
| | SEQ ID NO:355 | 3348 bp | |
| NOV127b, | CGTCCCGTGGCCATGACGACCGCTCAGAGGGACTCCCTGTTGTGGAAGCTCGCGGGGT | | |
| CG59971-02 DNA Sequence DNA Sequence | | | |
| | TGCTGCGGGAGTCCGGTGATGTGGTCCTGTCTGGCTGTAGCACCCTGAGCCTGCTGAC | | |
| | TCCCACACTGCAACAGCTGAACCACGTATTTGAGCTGCACCTGGGGCCATGGGGCCCT | | |
| | GGCCAGACAGGCTTTGTGGCTCTGCCCTCCCATCCTGCCGACTCCCCTGTTATTCTTC | | |
| | AGCTTCAGTTTCTCTTCGATGTGCTGCAGAAAACACTTTCACTCAAGCTGGTCCATGT | | |
| | TGCTGGTCCTGGCCCCACAGGGCCCATCAAGATTTTCCCCTTCAAATCCCTTCGGCAC | | |
| | CTGGAGCTCCGAGGTGTTCCCCTCCACTGTCTGCATGGCCTCCGAGGCATCTACTCCC | | |
| | AGCTGGAGACCCTGATTTGCAGCAGGAGCCTCCAGGCATTAGACGAGCTCCTCTCAGC | | |
| | CTGCGGCGGCGACTTCTGCTCTGCCCTCCCTTGGCTGGCTCTGCTTTCTGCCAACTTC | | |
| | AGCTACAATGCACTGACCGCCTTAGACAGCTCCCTGCGCCTCTTGTCAGCTCTGCGTT | | |
| | TCTTGAACCTAAGCCACAATCAAGTCCAGGACTGTCAGGGATTCCTGATGGATTTCTG | | |
| | TGAGCTCCACCATCTGGACATCTCCTATAATCGCCTGCATTTGGTGCCAAGAATGGGA | | |
| | CCCTCAGGGCCTGCTCTGGGGCTCCTGATACTGCGAGGCAATGAGCTTCGGAGCCTGC | | |
| | CAGGCCTAGAGCAGCTGAGGAATCTGCGGCACCTGGATTTGGCATACAACCTGCTGGA | | |
| | AGGACACCGGGAGCTGTCACCACTGTGGCTGCTGGCTGAGCTCCGCAAGCTCTACCTG | | |
| | GAGGGCAACCCTCTTTGGTTCCACCCTGAGCACCGAGCAGCCACTGCCCAGTACTTGT | | |
| | CACCCCGGGCCAGGGATGCTGCTACTGGCTTCCTTCTCGATGGCAAGGTCTTGTCACT | | |
| | GACAGATTTTCAGCAGACTCACACATCCTTGGGGCTCAGCCCCATGGGCCCACCTTTG | | |
| | AACCCCTCTCCGCCTCGTTGGTTCGTGCAGCAGCACCCGGAGCTGGAGCTCATGAGCA | | |
| | GCTTCCGGGAACGGTTCGGCCGCAACTGGCTGCAGTACAGGAGTCACCTGGAGCCCTC | | |
| | CGGAAACCCTCTGCCGGCCACCCCCACTACTTCTGCACCCAGTGCACCTCCAGCCAGC | | |
| | TCCCAGGGCCCCGACACTGCACCCAGACCTTCACCCCCGCAGGAGGAAGCCAGAGGCC | | |
| | CCCAGGAGTCACCACAGAAAATGTCAGAGGAGCTCAGGGCGGAGCCACAGGAGGAGGA | | |
| | AGACGAGAAGGAGGGGAAGGAGGAGAAGGAGGAGGGGGAGATGGTGGAACAGGGAGAA | | |
| | GAGGACGCAGGAGAGGAGGAAGAAGAGCAGCAGGACCAGAACGAAGTGGAACCGGAAC | | |
| | TCTGTCGCCCCTTGTTGGTCTGTCCCCTGGAGGGGCCTGAGGGCGTACGGGGCAGGGA | | |
| | ATGCTTTCTCAGGGTCACTTCTGCCCACCTGTTTGAGGTGGPACTCCAAGCAGCTCGC | | |
| | ACCTTGGAGCGACTGGAGCTCCAGAGTCTGGAGGCACCTGAGATAGAGCCGGAGGCCC | | |
| | ACGCCCAGAGGTCGCCCAGGCCCACGGGCTCAGATCTGCTCCCTGGAGCCCCCATGCT | | |
| | CAGTCTGCGCTTCTCCTACATCTGCCCTGACCGGCAGTTGCGTCGCTATTTGGTGCTG | | |
| | GAGCCTGATGCCCACGCAGCTCTCCAGGAGCTGCTTGCCGTGTTGACCCCAGTCACCA | | |
| | ATCTGGCTCGGCAACAGCTTGCCGACCCCAGGGACCTCCTGCTGGGTAGATTCCAGTG | | |
| | TCTACGCTCTGCCCATGACTTCAAGCCAGAGGAGCCCAGGATGGGATTAGACAGTGAG | | |
| | GAAGGCTCGAGGCCTCTGTTCCAAAAGACAGAATCTCCTGCTGTGTGTCCTAACTGTG | | |
| | CTAGTCACCACGTGCTTCTCCTCCCTCTCTCTCGGGGAACCCCCAACAGCGACCCGAA | | |
| | ACAGGCACAGCAGTCTCTGGCTCCTTCTCCGTCTGCCACCCCTCTCTGCCACCCTCCT | | |
| | GGCCATGCTCACCACCTTCACAGGGCCAACAACAGCCCACCTCACGCACCGACCACCC | | |
| | CTGACCATGGTAGTTGGAGCCTCAGTCCCGCCCGTGAGCGCTGTGGCCTCCGCTCTGT | | |
| | GGACCACCGACTCCGGCTCTTCCTGGATGTTGAGGTGTTCAGCGATGCCCACGAGGAG | | |
| | TTCCAGTGCTGCCTCAAGGTCCCACTCCCATTGGCAGGCCACACTGGGGAGTTCATGT | | |
| | GCCTTCTGCTTGTGTCTGACCGCAGCCTCTACCTGTTGAAGGTGACTGGGGAGATGAC | | |
| | TGACCCTCCAGCTACCTGGCTGCAGCTGACCCTGGCTCTTCCCCTCCAGGATCTGAGT | | |
| | GGCATACAGCTGGGCCTGGCAGGCCACAGCCTGCGGCTAGAGTGGGCAGCTCGGGCGG | | |
| | GCCGCTGTGTGCTCCTGCCCCGACATCCCAGCCATTGCCGGGCCTTCCTAGACGACCT | | |
| | CCTTGGTGTCTTGCAGTCTCTGCCCCCTCCCTGCAGGAACTGTGTCACTGCCACAGAG | | |
| | GAGGAGGTCACCCCCCACCACCGGCTCTGGCCATTCCTGGAAAAACACTCATCCTTGC | | |
| | AGGCTCCCCAGTTCTTCTACCTTCCGGCGTTCCTGGTTGAACCTGAAGCCTCTCTCCA | | |
| | GCTGATGCTTCCCTCCACCTGCCTCGTATCCCTGTTGCTGACTCCGTCCACCCTGTTC | | |
| | CTGTTAGATGAGGATGCTGCAGGGTCCCCGGCACAGCCCTCTCCTCCAGCAGCATCTG | | |
| | GCGAACCCTCTGAGAAGGTGCCTCCCTCGGGGCCCCGCCCTGCTGTGCGTGTCAGCCA | | |
| | GCAGCACCCACTCAGCAGCCTGAGCTCCGTGCTGCTCTACCGCTCAGCCCCTGAGGAC | | |
| | TTGCGGCTGCTCTTCTACGATGAGGTGTCCCGGCTGGAGAGCTTTTGGGCACTCCGTG | | |
| | TGGTGTGTCAGGAGCAGCTGACAGCCCTGCTTGCCTGGATCCGGGAACCATGGGAGGA | | |
| | GCTGTTTTCCATCGGACTCCGGACAGTGATCCAAGAGGCGCTGGCCCTTGACCGATGA | | |
| | GGGTCCCACGCTGACCTTGGCCCTGACCTCAGGAGCCACGCT | | |
| | ORF Start: ATG at 13 | | ORF Stop: TGA at 3304 |
| | SEQ ID NO:356 | 1097 aa | MW at 121064.1 kD |

| | |
|---|---|
| NOV127b, | MTTAQRDSLLWKLAGLLRESGDVVLSGCSTLSLLTPTLQQLNHVFELHLGPWGPGQTG |
| CG59971-02 Protein | |
| Sequence | FVALPSHPADSPVILQLQFLFDVLQKTLSLKLVHVAGPGPTGPIKIFPFKSLRHLELR |
| | GVPLHCLHGLRGIYSQLETLICSRSLQALEELLSACGGDFCSALPWLALLSANFSYNA |
| | LTALDSSLRLLSALRFLNLSHNQVQDCQGFLMDLCELHHLDISYNRLHLVPRMGPSGA |
| | ALGVLILRGNELRSLPGLEQLRNLRHLDLAYNLLEGHRELSPLWLLAELRKLYLEGNP |
| | LWFHPEHRAATAQYLSPRARDAATGFLLDGKVLSLTDFQQTHTSLGLSPMGPPLPWPV |
| | GSTPETSGGPDLSDSLSSGGVVTQPLLHKVKSRVRVRRASISEPSDTDPEPRTLNPSP |
| | AGWFVQQHPELELMSSFRERFGRNWLQYRSHLEPSGNPLPATPTTSAPSAPPASSQGP |
| | DTAPRPSPPQEEARGPQESPQKMSEEVRAEPQEEEEEKEGKEEKEEGEMVEQGEEEAG |
| | EEEEEEQDQKEVEAELCRPLLVCPLEGPEGVRGRECFLRVTSAHLFEVELQAARTLER |
| | LELQSLEAAEIEPEAQAQGPPLATGSDLLPGAPILSLRFSYICPDRQLRRYLVLEPDA |
| | HAAVQELLAVLTPVTNVAREQLGEARDLLLGRFQCLRCGHEFKPEEPRMGLDSEEGWR |
| | PLFQKTESPAVCPNCGSDHVVLLAVSRGTPNRERKQGEQSLAPSPSASPVCHPPGHGD |
| | HLDRAKNSPPQAPSTRDHGSWSLSPAPERCGLRSVDHRLRLFLDVEVFSDAQEEFQCC |
| | LKVPVALAGHTGEFMCLVVVSDRRLYLLKVTGEMSEPPASWLQLTLAVPLQDLSGIEL |
| | GLAGQSLRLEWAAGAGRCVLLPRDARHCRAFLEELLGVLQSLPPAWRNCVSATEEEVT |
| | PQHRLWPLLEKDSSLEARQFFYLRAFLVEGEASVQLMLPSTCLVSLLLTPSTLFLLDE |
| | DAAGSPAEPSPPAASGEASEKVPPSGPGPAVRVREQQPLSSLSSVLLYRSAPEDLRLL |
| | FYDEVSRLESFWALRVVCQEQLTALLAWIREPWEELFSIGLRTVIQEALALDR |

Sequence comparison of the above protein sequences yields the following sequence relationships shown in Table 127B.

TABLE 127B
Comparison of NOV127a against NOV127b.

| | | Identities/ |
|---|---|---|
| | | Similarities |
| Protein | NOV127a Residues/ | for the |
| Sequence | Match Residues | Matched Region |
| NOV127b | 1 . . . 1098 | 891/1098 (81%) |
| | 1 . . . 1097 | 891/1098 (81%) |

Further analysis of the NOV127a protein yielded the following properties shown in Table 127C.

TABLE 127C
Protein Sequence Properties NOV127a

| | |
|---|---|
| PSort | 0.5163 probability located in mitochondrial matrix space; |
| analysis: | 0.3000 probability located in microbody (peroxisome); 0.2442 |
| | probability located in mitochondrial inner membrane; 0.2442 |
| | probability located in mitochondrial intermembrane space |
| SignalP | No Known Signal Sequence Predicted |
| analysis: | |

A search of the NOV127a protein against the Geneseq database, a proprietary database that contains sequences published in patents and patent publication, yielded several homologous proteins shown in Table 127D.

TABLE 127D
Geneseq Results for NOV127a

| | | NOV127a | Identities/ | | |
|---|---|---|---|---|---|
| | | Residues/ | Similarities | | |
| Geneseq | Protein/Organism/Length | Match | for the | | Expect |
| Identifier | [Patent #, Date] | Residues | Matched Region | | Value |
| AAM39827 | Human polypeptide SEQ ID NO | 375 . . . 528 | 140/154 | (90%) | 3e−78 |
| | 2972 - Homo sapiens, 169 aa. | 14 . . . 167 | 145/154 | (93%) | |
| | [WO200153312-A1, 26 JUL 2001] | | | | |
| AAM41613 | Human polypeptide SEQ ID NO | 375 . . . 528 | 140/154 | (90%) | 4e−78 |
| | 6544 - Homo sapiens, 184 aa. | 29 . . . 182 | 145/154 | (93%) | |
| | [WO200153312-A1, 26 JUL 2001] | | | | |
| AAU19764 | Human novel extracellular matrix | 444 . . . 647 | 157/207 | (75%) | 2e−75 |
| | protein, Seq ID No 414 - Homo | 13 . . . 209 | 160/207 | (76%) | |
| | sapiens, 211 aa. [WO200155368- | | | | |
| | A1, 2 AUG 2001] | | | | |
| ABB19833 | Protein #1832 encoded by probe | 409 . . . 535 | 127/127 | (100%) | 2e−70 |
| | for measuring heart cell gene | 1 . . . 127 | 127/127 | (100%) | |
| | expression - Homo sapiens, 127 aa. | | | | |
| | [WO200157274-A2, 9 AUG | | | | |
| | 2001] | | | | |
| AAM67606 | Human bone marrow expressed | 409 . . . 535 | 127/127 | (100%) | 2e−70 |
| | probe encoded protein SEQ ID | 1 . . . 127 | 127/127 | (100%) | |
| | NO: 27912 - Homo sapiens, 127 | | | | |
| | aa. [WO200157276-A2, 9 AUG | | | | |
| | 2001] | | | | |

In a BLAST search of public sequence databases, the NOV127a protein was found to have homology to the proteins shown in the BLASTP data in Table 127E.

TABLE 127E
Public BLASTP Results for NOV127a

| | | NOV127a | Identities/ | | |
|---|---|---|---|---|---|
| Protein | | Residues/ | Similarities | | |
| Accession | | Match | for the | | Expect |
| Number | Protein/Organism/Length | Residues | Matched Portion | | Value |
| AAL49726 | LKB1-INTERACTING | 1 . . . 1098 | 1077/1098 | (98%) | 0.0 |
| | PROTEIN 1 - Homo sapiens | 12 . . . 1099 | 1078/1098 | (98%) | |
| | (Human), 1099 aa. | | | | |
| Q96PY9 | KIAA1898 PROTEIN - Homo | 76 . . . 1098 | 1003/1023 | (98%) | 0.0 |
| | sapiens (Human), 1013 aa | 1 . . . 1013 | 1003/1023 | (98%) | |
| | (fragment). | | | | |
| Q96CN3 | SIMILAR TO RIKEN CDNA | 288 . . . 1098 | 793/811 | (97%) | 0.0 |
| | 1200014D22 GENE - Homo | 4 . . . 804 | 793/811 | (97%) | |
| | sapiens (Human), 804 aa | | | | |
| | (fragment). | | | | |
| Q9DBT7 | 1200014D22RIK PROTEIN - | 1 . . . 1098 | 816/1098 | (74%) | 0.0 |
| | Mus musculus (Mouse), 1072 aa. | 1 . . . 1072 | 895/1098 | (81%) | |
| Q9VMK9 | CG9044 PROTEIN - Drosophila | 12 . . . 433 | 139/459 | (30%) | 6e−38 |
| | melanogaster (Fruit fly), 1289 aa. | 8 . . . 463 | 220/459 | (47%) | |

PFam analysis predicts that the NOV127a protein contains the domains shown in the Table 127F.

TABLE 127F
Domain Analysis of NOV127a

| | | Identities/ | | |
|---|---|---|---|---|
| | | Similarities | | |
| Pfam | NOV127a | for the | | Expect |
| Domain | Match Region | Matched Region | | Value |
| LRR: domain 1 of 5 | 164 . . . 186 | 7/25 | (28%) | 2.5e+02 |
| | | 15/25 | (60%) | |
| LRR: domain 2 of 5 | 187 . . . 209 | 6/25 | (24%) | 2.5e+02 |
| | | 16/25 | (64%) | |
| LRR: domain 3 of 5 | 210 . . . 231 | 8/25 | (32%) | 83 |
| | | 13/25 | (52%) | |
| LRR: domain 4 of 5 | 233 . . . 254 | 9/25 | (36%) | 16 |
| | | 17/25 | (68%) | |
| LRR: domain 5 of 5 | 255 . . . 279 | 10/27 | (37%) | 22 |
| | | 19/27 | (70%) | |
| Pkinase_C: | 620 . . . 629 | 5/11 | (45%) | 8.9 |
| domain 1 of 1 | | 9/11 | (82%) | |
| rubredoxin: | 669 . . . 686 | 5/18 | (28%) | 4.6 |
| domain 1 of 2 | | 13/18 | (72%) | |
| rubredoxin: | 708 . . . 713 | 5/6 | (83%) | 1.2e+03 |
| domain 2 of 2 | | 6/6 | (100%) | |

### Example B

### Sequencing Methodology and Identofication of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology:

The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. Table B1 shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone:

Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoimmune diseases), Panel CNSD.01 (containing central nervous system samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v0.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.31).

Panels 2D and 2.2

The plates for Panels 2D and 2.2 generally include 2 control wells and 94 test samples; composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PEA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSFs and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection column!; and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMBM 5% FCS (Hyclone), 100AM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 ug/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 101 g/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 1001M non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (11 g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵cells/ml for 8 days, changing the media every 3 days and adjusting the cell concentration to 5×10⁵cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

IDA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels: 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodeeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG58522-01: Human Platelet-Activating Factor Acetylhydrolase Ib Beta

Expression of gene CG58522-01 was assessed using the primer-probe set Ag3365, described in Table AA. Results of the RTQ-PCR runs are shown in Table AB.

Table AA. Probe Name Ag3365

TABLE AA
Probe Name Ag3365

| | | | Start | SEQ ID |
|---|---|---|---|---|
| Primers | Sequences | Length | Position | NO: |
| Forward | 5′-cagaatgaaccaaggagactca-3′ | 22 | 3 | 357 |
| Probe | TET-5′-ctactccgcatgcggcagaagacatt-3′-TAMRA | 26 | 35 | 358 |
| Reverse | 5′-cacatccatctgtcatctcctt-3′ | 22 | 62 | 359 |

TABLE AB
General_screening_panel_v1.4

| | Rel. Exp. | | Rel. Exp. |
|---|---|---|---|
| | (%) Ag3365, | | (%) Ag3365, |
| | Run | | Run |
| Tissue Name | 216709759 | Tissue Name | 216709759 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 10.7 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 4.9 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 7.9 | Stomach Pool | 0.0 |
| IGROV-1 | | | |
| Ovarian ca. | 26.8 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 1.7 | Lymph Node Pool | 16.5 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 3.3 | CNS cancer (astro) | 0.0 |
| | | SNB-75 | |
| Lung ca. NCI-H146 | 4.5 | CNS cancer (glio) | 6.2 |
| | | SNB-19 | |
| Lung ca. SHP-77 | 0.0 | CNS cancer (glio) SF- | 25.7 |
| | | 295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 100.0 | Brain (fetal) | 0.0 |
| Lung ca. NCI-H460 | 0.0 | Brain (Hippocampus) | 4.8 |
| | | Pool | |
| Lung ca. HOP-62 | 0.0 | Cerebral Cortex Pool | 0.0 |
| Lung ca. NCI-H522 | 0.0 | Brain (Substantia | 1.8 |
| | | nigra) Pool | |
| Liver | 0.0 | Brain (Thalamus) Pool | 3.6 |
| Fetal Liver | 0.0 | Brain (whole) | 6.9 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

CNS_neurodegeneration_v1.0 Summary: Ag3365—Expression of this gene is low/undetectable (CTs>35) across all of the samples on this panel (data not shown).

General_screening_panel_v1.4 Summary: Ag3365—Significant expression of this gene is seen only in the lung cancer cell line NCI-H23 (CT=33.1). Therefore, expression of this gene may be used to distinguish this sample from the other samples on this panel.

Panel 4D Summary: Ag3365—Expression of this gene is low/undetectable (CTs>35) across all of the samples on this panel (data not shown).

B. CG58520-01: Gamma-Aminobutyric-Acid Receptor Gamma-1

Expression of gene CG58520-01 was assessed using the primer-probe set Ag3364, described in Table BA.

Table BA. Probe Name Ag3364

TABLE BA
Probe Name Ag3364

| | | | Start | SEQ ID |
|---|---|---|---|---|
| Primers | Sequences | Length | Position | NO: |
| Forward | 5′-ttcttctgcggagtcaaagtag-3′ | 22 | 43 | 360 |
| Probe | TET-5′-ttggtcttcttgttactgaccctgca-3′-TAMRA | 26 | 75 | 361 |
| Reverse | 5′-tcatctgccttatcaacgtttc-3′ | 22 | 106 | 362 |

CNS_neurodegeneration_v1.0 Summary: Ag3364—Expression of this gene is low/undetectable (CTs>35) across all of the samples on this panel (data not shown).

General_screening_panel_v1.4 Summary: Ag3364—Expression of this gene is low/undetectable (CTs>35) across all of the samples on this panel (data not shown).

Panel 4D Summary: Ag3364—Expression of this gene is low/undetectable (CTs>35) across all of the samples on this panel (data not shown).

Panel CNS_{—}1 Summary: Ag3364—Expression of this gene is low/undetectable (CTs>35) across all of the samples on this panel (data not shown).

C. CG58520-03: Gamma-Aminobutyric-Acid Receptor Gamma-1 Subunit Precursor (Gaba(A) Receptor)

Expression of gene CG58520-03 was assessed using the primer-probe set Ag5092, described in Table CA.

Table CA. Probe Name Ag5092

TABLE CA
Probe Name Ag5092

| | | | Start | SEQ ID |
|---|---|---|---|---|
| Primers | Sequences | Length | Position | NO: |
| Forward | 5′-gaacattcctgtccactgga-3′ | 20 | 625 | 363 |
| Probe | TET-5′-attttcaagcgatggataccctaaaa-3′-TAMRA | 26 | 645 | 364 |
| Reverse | 5′-cacttctacggagggctttt-3′ | 20 | 692 | 365 |

CNS_neurodegeneration_v1.0 Summary: Ag5092—Expression of this gene is low/undetectable (CTs>35) across all of the samples on this panel (data not shown).

General_screening_panel_v1.5 Summary: Ag5092—Expression of this gene is low/undetectable (CTs>35) across all of the samples on this panel (data not shown).

Panel 4.1D Summary: Ag5092—Expression of this gene is low/undetectable (CTs>35) across all of the samples on this panel (data not shown).

D. CG58518-01: Gamma-Aminobutyric-Acid Receptor RHO-3

Expression of gene CG58518-01 was assessed using the primer-probe sets Ag3363, Ag1130, Ag1198, Ag1253 and Ag1603, described in Tables DA, DB, DC, DD and DE. Results of the RTQ-PCR runs are shown in Tables DF, DG and DH.

Table DA. Probe Name Ag3363

TABLE DA
Probe Name Ag3363

| | | | Start | SEQ ID |
|---|---|---|---|---|
| Primers | Sequences | Length | Position | NO: |
| Forward | 5′-tggctttccagttagtctcctt-3′ | 22 | 14 | 366 |
| Probe | TET-5′-cacctacatctggatcatattgaaacca-3′-TAMRA | 28 | 36 | 367 |
| Reverse | 5′-ttgatgttagaagcagcacaaa-3′ | 22 | 68 | 368 |

Table DB. Probe Name Ag1130

TABLE DB
Probe Name Ag1130

| | | | Start | SEQ ID |
|---|---|---|---|---|
| Primers | Sequences | Length | Position | NO: |
| Forward | 5′-gtcctggctttccagttagtct-3′ | 22 | 10 | 369 |
| Probe | TET-5′-tcacctacatctggatcatattgaaacca-3′-TAMRA | 29 | 35 | 370 |
| Reverse | 5′-ttgatgttagaagcagcacaaa-3′ | 22 | 68 | 371 |

Table DC. Probe Name Ag1198

TABLE DC
Probe Name Ag1198

| | | | Start | SEQ ID |
|---|---|---|---|---|
| Primers | Sequences | Length | Position | NO: |
| Forward | 5′-gtcctggctttccagttagtct-3′ | 22 | 10 | 372 |
| Probe | TET-5′-tcacctacatctggatcatattgaaacca-3′-TAMRA | 29 | 35 | 373 |
| Reverse | 5′-ttgatgttagaagcagcacaaa-3′ | 22 | 68 | 374 |

Table DD. Probe Name Ag1253

TABLE DD
Probe Name Ag1253

| | | | Start | SEQ ID |
|---|---|---|---|---|
| Primers | Sequences | Length | Position | NO: |
| Forward | 5′-atctgggtgcctgatatctttt-3′ | 22 | 466 | 375 |
| Probe | TET-5′-tgtccactctaaaagatccttcatccatga-3′-TAMRA | 30 | 489 | 376 |
| Reverse | 5′-cgcagcatgatattctccatag-3′ | 22 | 524 | 377 |

Table DE. Probe Name Ag1603

TABLE DE
Probe Name Ag1603

| | | | Start | SEQ ID |
|---|---|---|---|---|
| Primers | Sequences | Length | Position | NO: |
| Forward | 5′-gtcctggctttccagttagtct-3′ | 22 | 10 | 378 |
| Probe | TET-5′-tcacctacatctggatcatattgaaacca-3′-TAMRA | 29 | 35 | 379 |
| Reverse | 5′-ttgatgttagaagcagcacaaa-3′ | 22 | 68 | 380 |

TABLE DF
General_screening_panel_v1.4

| | Rel. Exp. | | Rel. Exp. |
|---|---|---|---|
| | (%) Ag3363, | | (%) Ag3363, |
| | Run | | Run |
| Tissue Name | 216709559 | Tissue Name | 216709559 |
| Adipose | 0.0 | Renal ca. TK-10 | 0.0 |
| Melanoma* | 0.0 | Bladder | 6.6 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 16.7 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 6.8 |
| MB-231 | | | |
| Breast ca. BT 549 | 0.0 | Fetal Skeletal Muscle | 0.0 |
| Breast ca. T47D | 6.4 | Skeletal Muscle Pool | 0.0 |
| Breast ca. MDA-N | 0.0 | Spleen Pool | 8.5 |
| Breast Pool | 0.0 | Thymus Pool | 0.0 |
| Trachea | 0.0 | CNS cancer | 0.0 |
| | | (glio/astro) U87-MG | |
| Lung | 0.0 | CNS cancer | 10.9 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 77.9 | CNS cancer (glio) | 0.0 |
| | | SNB-19 | |
| Lung ca. SHP-77 | 100.0 | CNS cancer (glio) SF- | 11.4 |
| | | 295 | |
| Lung ca. A549 | 10.1 | Brain (Amygdala) | 0.0 |
| | | Pool | |
| Lung ca. NCI-H526 | 0.0 | Brain (cerebellum) | 0.0 |
| Lung ca. NCI-H23 | 34.4 | Brain (fetal) | 0.0 |
| Lung ca. NCI-H460 | 30.6 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 0.0 | Brain (Thalamus) Pool | 5.1 |
| Fetal Liver | 0.0 | Brain (whole) | 50.0 |
| Liver ca. HepG2 | 0.0 | Spinal Cord Pool | 0.0 |
| Kidney Pool | 3.0 | Adrenal Gland | 0.0 |
| Fetal Kidney | 8.4 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

TABLE DG
Panel 1.2

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1130, | Ag1130, | Ag1198, | | Ag1130, | Ag1130, | Ag1198, |
| Tissue | Run | Run | Run | Tissue | Run | Run | Run |
| Name | 125117140 | 126566764 | 129140506 | Name | 125117140 | 126566764 | 129140506 |
| Endothelial | 0.0 | 0.0 | 0.0 | Renal ca. | 0.0 | 0.0 | 0.0 |
| cells | | | | 786-0 | | | |
| Heart | 0.0 | 0.0 | 0.0 | Renal ca. | 7.3 | 4.7 | 0.0 |
| (Fetal) | | | | A498 | | | |
| Pancreas | 0.0 | 0.0 | 0.0 | Renal ca. | 0.0 | 0.0 | 0.0 |
| | | | | RXF 393 | | | |
| Pancreatic | 9.0 | 0.0 | 0.0 | Renal ca. | 0.0 | 0.0 | 0.0 |
| ca. CAPAN 2 | | | | ACHN | | | |
| Adrenal | 0.0 | 2.6 | 0.0 | Renal ca. | 3.9 | 0.0 | 0.0 |
| Gland | | | | UO-31 | | | |
| Thyroid | 0.0 | 0.0 | 0.0 | Renal ca. | 0.0 | 0.0 | 0.0 |
| | | | | TK-10 | | | |
| Salivary | 0.0 | 0.0 | 0.0 | Liver | 26.6 | 0.0 | 0.0 |
| gland | | | | | | | |
| Pituitary | 0.0 | 0.0 | 0.0 | Liver | 25.3 | 0.0 | 0.0 |
| gland | | | | (fetal) | | | |
| Brain | 0.0 | 0.0 | 0.0 | Liver ca. | 0.0 | 0.0 | 0.0 |
| (fetal) | | | | (hepatoblast) | | | |
| | | | | HepG2 | | | |
| Brain | 2.6 | 20.0 | 0.0 | Lung | 0.0 | 0.0 | 0.0 |
| (whole) | | | | | | | |
| Brain | 1.3 | 32.1 | 0.0 | Lung | 0.0 | 0.0 | 0.0 |
| (amygdala) | | | | (fetal) | | | |
| Brain | 1.5 | 3.8 | 0.0 | Lung ca. | 3.4 | 0.0 | 0.0 |
| (cerebellum) | | | | (small | | | |
| | | | | cell) LX-1 | | | |
| Brain | 0.0 | 27.0 | 0.0 | Lung ca. | 28.5 | 74.2 | 0.0 |
| (hippocampus) | | | | (small | | | |
| | | | | cell) NCI- | | | |
| | | | | H69 | | | |
| Brain | 9.9 | 22.5 | 9.8 | Lung ca. | 3.8 | 9.7 | 0.0 |
| (thalamus) | | | | (s.cell | | | |
| | | | | var.) | | | |
| | | | | SHP-77 | | | |
| Cerebral | 0.0 | 0.0 | 0.0 | Lung ca. | 8.8 | 4.1 | 5.3 |
| Cortex | | | | (large | | | |
| | | | | cell)NCI- | | | |
| | | | | H460 | | | |
| Spinal cord | 4.4 | 0.0 | 0.0 | Lung ca. | 51.4 | 9.5 | 7.2 |
| | | | | (non-sm. | | | |
| | | | | cell) A549 | | | |
| glio/astro | 0.0 | 0.0 | 0.0 | Lung ca. | 0.0 | 0.0 | 0.0 |
| U87-MG | | | | (non- | | | |
| | | | | s.cell) | | | |
| | | | | NCI-H23 | | | |
| glio/astro | 0.0 | 0.0 | 0.0 | Lung ca. | 8.4 | 2.7 | 9.6 |
| U-118-MG | | | | (non- | | | |
| | | | | s.cell) | | | |
| | | | | HOP-62 | | | |
| astrocytoma | 2.9 | 0.0 | 0.0 | Lung ca. | 0.0 | 0.0 | 0.0 |
| SW1783 | | | | (non-s.cl) | | | |
| | | | | NCI- | | | |
| | | | | H522 | | | |
| neuro*; met | 0.0 | 0.0 | 0.0 | Lung ca. | 3.2 | 8.7 | 0.0 |
| SK-N-AS | | | | (squam.) | | | |
| | | | | SW 900 | | | |
| astrocytoma | 5.1 | 0.0 | 0.0 | Lung ca. | 2.3 | 15.9 | 0.0 |
| SF-539 | | | | (squam.) | | | |
| | | | | NCI- | | | |
| | | | | H596 | | | |
| astrocytoma | 2.3 | 0.0 | 0.0 | Mammary | 0.0 | 0.0 | 0.0 |
| SNB-75 | | | | gland | | | |
| glioma | 6.3 | 20.7 | 9.0 | Breast | 0.0 | 0.0 | 0.0 |
| SNB-19 | | | | ca.* | | | |
| | | | | (pl.ef) | | | |
| | | | | MCF-7 | | | |
| glioma | 1.4 | 0.0 | 1.8 | Breast | 0.0 | 0.0 | 0.0 |
| U251 | | | | ca.* | | | |
| | | | | (pl.ef) | | | |
| | | | | MDA- | | | |
| | | | | MB-231 | | | |
| glioma SF- | 0.0 | 0.0 | 0.0 | Breast | 14.1 | 37.4 | 0.0 |
| 295 | | | | ca.* (pl. | | | |
| | | | | ef) T47D | | | |
| Heart | 0.0 | 0.0 | 0.0 | Breast ca. | 12.5 | 21.0 | 12.3 |
| | | | | BT-549 | | | |
| Skeletal | 2.3 | 0.0 | 0.0 | Breast ca. | 0.0 | 0.0 | 0.0 |
| Muscle | | | | MDA-N | | | |
| Bone | 0.0 | 0.0 | 0.0 | Ovary | 0.0 | 0.0 | 0.0 |
| marrow | | | | | | | |
| Thymus | 0.0 | 0.0 | 0.0 | Ovarian | 0.0 | 0.0 | 0.0 |
| | | | | ca. | | | |
| | | | | OVCAR-3 | | | |
| Spleen | 2.2 | 0.0 | 0.0 | Ovarian | 0.0 | 0.0 | 0.0 |
| | | | | ca. | | | |
| | | | | OVCAR-4 | | | |
| Lymph | 0.0 | 0.0 | 0.0 | Ovarian | 66.9 | 35.4 | 4.4 |
| node | | | | ca. | | | |
| | | | | OVCAR-5 | | | |
| Colorectal | 11.3 | 27.7 | 21.8 | Ovarian | 2.7 | 0.0 | 0.0 |
| Tissue | | | | ca. | | | |
| | | | | OVCAR-8 | | | |
| Stomach | 0.0 | 0.0 | 0.0 | Ovarian | 6.0 | 0.0 | 0.0 |
| | | | | ca. | | | |
| | | | | IGROV-1 | | | |
| Small | 5.4 | 0.0 | 0.0 | Ovarian | 30.8 | 0.0 | 0.0 |
| intestine | | | | ca. | | | |
| | | | | (ascites) | | | |
| | | | | SK-OV-3 | | | |
| Colon ca. | 3.2 | 0.0 | 0.0 | Uterus | 0.0 | 0.0 | 0.0 |
| SW480 | | | | | | | |
| Colon ca.* | 0.0 | 0.0 | 0.0 | Placenta | 0.0 | 0.0 | 0.0 |
| SW620 | | | | | | | |
| (SW480 | | | | | | | |
| met) | | | | | | | |
| Colon ca. | 1.9 | 14.4 | 0.0 | Prostate | 6.9 | 0.0 | 0.0 |
| HT29 | | | | | | | |
| Colon ca. | 0.0 | 0.0 | 0.0 | Prostate | 100.0 | 0.0 | 0.0 |
| HCT-116 | | | | ca.* (bone | | | |
| | | | | met) PC-3 | | | |
| Colon ca. | 0.0 | 0.0 | 0.0 | Testis | 54.7 | 100.0 | 36.9 |
| CaCo-2 | | | | | | | |
| Colon ca. | 72.2 | 75.8 | 100.0 | Melanoma | 4.2 | 0.0 | 0.0 |
| Tissue | | | | Hs688(A).T | | | |
| (ODO3866) | | | | | | | |
| Colon ca. | 5.3 | 4.8 | 0.0 | Melanoma* | 2.7 | 34.2 | 13.3 |
| HCC-2998 | | | | (met) | | | |
| | | | | Hs688(B).T | | | |
| Gastric ca.* | 50.3 | 0.0 | 0.0 | Melanoma | 0.0 | 0.0 | 0.0 |
| (liver met) | | | | UACC- | | | |
| NCI-N87 | | | | 62 | | | |
| Bladder | 6.0 | 22.1 | 0.0 | Melanoma | 31.4 | 36.3 | 20.2 |
| | | | | M14 | | | |
| Trachea | 0.0 | 0.0 | 0.0 | Melanoma | 0.0 | 0.0 | 0.0 |
| | | | | LOX | | | |
| | | | | IMVI | | | |
| Kidney | 2.0 | 0.0 | 0.0 | Melanoma* | 2.4 | 0.0 | 0.0 |
| | | | | (met) | | | |
| | | | | SK-MEL-5 | | | |
| Kidney | 1.1 | 2.5 | 0.0 | | | | |
| (fetal) | | | | | | | |

TABLE DH
Panel 4R

| | Rel. Exp. (%) | | Rel. Exp. (%) |
|---|---|---|---|
| | Ag1198, Run | | Ag1198, Run |
| Tissue Name | 142014937 | Tissue Name | 142014937 |
| Secondary Th1 act | 0.0 | HUVEC IL-1beta | 0.0 |
| Secondary Th2 act | 0.0 | HUVEC IFN gamma | 0.0 |
| Secondary Tr1 act | 2.5 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 16.4 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 1 | 100.0 |
| CD40 | | | |
| Monocytes rest | 0.0 | IBD Colitis 2 | 0.0 |
| Monocytes LPS | 0.0 | IBD Crohn's | 0.0 |
| Macrophages rest | 0.0 | Colon | 0.0 |
| Macrophages LPS | 0.0 | Lung | 0.0 |
| HUVEC none | 0.0 | Thymus | 0.0 |
| HUVEC starved | 0.0 | Kidney | 0.0 |

CNS_neurodegeneration_v1.0 Summary: Ag3363—Expression of this gene is low/undetectable (CTs>35) across all of the samples on this panel (data not shown).

General_screening_panel_v1.4 Summary: Ag3363—Significant expression is seen in lung cancer cell line NCI-H1146 (CT=34.5) and lung cancer cell line SHP-77 (CT=34.2). Therefore, expression of this can be used to distinguish these samples from the rest of the samples on this panel.

Panel 1.2 Summary: Ag1130/Ag1198—Three different runs using the same primer sequences yield similar results. Significant expression of this gene is seen in testis and a colon cancer sample. Therefore, expression of this gene can be used to differentiate these samples from other samples on these panels. Results from a third experiment using the probe and primer set Ag1253 show low/undetectable levels of expression in all the samples on this panel.

Panel 1.3D Summary: Ag1253—Expression of this gene is low/undetectable (CTs>35) across all of the samples on this panel (data not shown).

Panel 2D Summary: Ag1603—Expression of this gene is low/undetectable (CTs>35) across all of the samples on this panel (data not shown)

Panel 4D Summary: Ag1130/Ag1198/Ag1253/Ag3363—Two experiments showed possible experimental difficulties, while the other three runs showed expression of this gene as low/undetectable (CTs>35) across all of the samples on the panel.

Panel 4R Summary: Ag1198—Significant expression of this gene is seen only in the IBD colitis 1 sample (CT=34.2). Therefore, expression of this gene can be used to differentiate this sample from others on the panel.

Panel CNS 1 Summary: Ag1253/Ag1603—Expression of this gene is low/undetectable (CTs>35) across all of the samples on this panel (data not shown).

E. CG58516-01: G-Protein Beta WD-40 Repeats

Expression of gene CG58516-01 was assessed using the primer-probe set Ag3362, described in Table EA. Results of the RTQ-PCR runs are shown in Tables EB and EC.

Table EA. Probe Name Ag3362

TABLE EA
Probe Name Ag3362

| | | | Start | SEQ ID |
|---|---|---|---|---|
| Primers | Sequences | Length | Position | NO: |
| Forward | 5′-gtcgggcaggacctttact-3′ | 19 | 1474 | 381 |
| Probe | TET-5′-tcctacagctaattctgcagggcaca-3′-TAMRA | 26 | 1498 | 382 |
| Reverse | 5′-tacgctttactcccgtaagtca-3′ | 22 | 1543 | 383 |

TABLE EB
CNS_neurodegeneration_v1.0

| | | | Rel. Exp. |
|---|---|---|---|
| | Rel. Exp. (%) | | (%) Ag3362, |
| | Ag3362, Run | | Run |
| Tissue Name | 210153738 | Tissue Name | 210153738 |
| AD 1 Hippo | 9.9 | Control (Path) 3 | 0.0 |
| | | Temporal Ctx | |
| AD 2 Hippo | 33.2 | Control (Path) 4 | 24.3 |
| | | Temporal Ctx | |
| AD 3 Hippo | 4.3 | AD 1 Occipital | 2.0 |
| | | Ctx | |
| AD 4 Hippo | 16.5 | AD 2 Occipital | 0.0 |
| | | Ctx (Missing) | |
| AD 5 hippo | 96.6 | AD 3 Occipital | 5.4 |
| | | Ctx | |
| AD 6 Hippo | 43.2 | AD 4 Occipital | 24.7 |
| | | Ctx | |
| Control 2 Hippo | 29.1 | AD 5 Occipital | 24.5 |
| | | Ctx | |
| Control 4 Hippo | 16.6 | AD 6 Occipital | 31.9 |
| | | Ctx | |
| Control (Path) 3 | 3.8 | Control 1 Occipital | 0.9 |
| Hippo | | Ctx | |
| AD 1 Temporal Ctx | 7.1 | Control 2 Occipital | 89.5 |
| | | Ctx | |
| AD 2 Temporal Ctx | 23.2 | Control 3 Occipital | 12.6 |
| | | Ctx | |
| AD 3 Temporal Ctx | 5.6 | Control 4 Occipital | 6.3 |
| | | Ctx | |
| AD 4 Temporal Ctx | 20.0 | Control (Path) 1 | 65.1 |
| | | Occipital Ctx | |
| AD 5 Inf Temporal | 100.0 | Control (Path) 2 | 15.8 |
| Ctx | | Occipital Ctx | |
| AD 5 SupTemporal | 43.8 | Control (Path) 3 | 2.0 |
| Ctx | | Occipital Ctx | |
| AD 6 Inf Temporal | 30.8 | Control (Path) 4 | 11.6 |
| Ctx | | Occipital Ctx | |
| AD 6 Sup Temporal | 69.7 | Control 1 Parietal | 2.8 |
| Ctx | | Ctx | |
| Control 1 Temporal | 9.0 | Control 2 Parietal | 39.2 |
| Ctx | | Ctx | |
| Control 2 Temporal | 59.0 | Control 3 Parietal | 23.5 |
| Ctx | | Ctx | |
| Control 3 Temporal | 11.7 | Control (Path) 1 | 69.7 |
| Ctx | | Parietal Ctx | |
| Control 4 Temporal | 8.2 | Control (Path) 2 | 14.9 |
| Ctx | | Parietal Ctx | |
| Control (Path) 1 | 56.3 | Control (Path) 3 | 0.9 |
| Temporal Ctx | | Parietal Ctx | |
| Control (Path) 2 | 34.2 | Control (Path) 4 | 38.7 |
| Temporal Ctx | | Parietal Ctx | |

TABLE EC
General_screening_panel_v1.4

| | Rel. Exp. | | Rel. Exp. |
|---|---|---|---|
| | (%) Ag3362, | | (%) Ag3362, |
| | Run | | Run |
| Tissue Name | 216523482 | Tissue Name | 216523482 |
| Adipose | 6.3 | Renal ca. TK-10 | 44.1 |
| Melanoma* | 17.6 | Bladder | 9.4 |
| Hs688(A).T | | | |
| Melanoma* | 18.3 | Gastric ca. (liver met.) | 21.6 |
| Hs688(B).T | | NCI-N87 | |
| Melanoma* M14 | 17.1 | Gastric ca. KATO III | 17.6 |
| Melanoma* | 13.6 | Colon ca. SW-948 | 5.8 |
| LOXIMVI | | | |
| Melanoma* SK- | 19.6 | Colon ca. SW480 | 34.6 |
| MEL-5 | | | |
| Squamous cell | 14.6 | Colon ca.* (SW480 | 14.2 |
| carcinoma SCC-4 | | met) SW620 | |
| Testis Pool | 4.0 | Colon ca. HT29 | 7.2 |
| Prostate ca.* (bone | 90.8 | Colon ca. HCT-116 | 14.3 |
| met) PC-3 | | | |
| Prostate Pool | 4.0 | Colon ca. CaCo-2 | 19.8 |
| Placenta | 11.4 | Colon cancer tissue | 3.6 |
| Uterus Pool | 2.1 | Colon ca. SW1116 | 9.4 |
| Ovarian ca. | 17.4 | Colon ca. Colo-205 | 8.8 |
| OVCAR-3 | | | |
| Ovarian ca. SK- | 47.0 | Colon ca. SW-48 | 13.2 |
| OV-3 | | | |
| Ovarian ca. | 14.7 | Colon Pool | 5.7 |
| OVCAR-4 | | | |
| Ovarian ca. | 31.6 | Small Intestine Pool | 10.2 |
| OVCAR-5 | | | |
| Ovarian ca. | 12.9 | Stomach Pool | 6.2 |
| IGROV-1 | | | |
| Ovarian ca. | 6.7 | Bone Marrow Pool | 1.3 |
| OVCAR-8 | | | |
| Ovary | 12.5 | Fetal Heart | 1.1 |
| Breast ca. MCF-7 | 75.8 | Heart Pool | 3.4 |
| Breast ca. MDA- | 30.4 | Lymph Node Pool | 8.7 |
| MB-231 | | | |
| Breast ca. BT 549 | 65.5 | Fetal Skeletal Muscle | 2.3 |
| Breast ca. T47D | 100.0 | Skeletal Muscle Pool | 9.4 |
| Breast ca. MDA-N | 33.4 | Spleen Pool | 4.6 |
| Breast Pool | 4.6 | Thymus Pool | 7.3 |
| Trachea | 7.7 | CNS cancer | 33.9 |
| | | (glio/astro) U87-MG | |
| Lung | 4.9 | CNS cancer | 27.2 |
| | | (glio/astro) U-118-MG | |
| Fetal Lung | 7.1 | CNS cancer | 16.0 |
| | | (neuro; met) SK-N-AS | |
| Lung ca. NCI-N417 | 9.3 | CNS cancer (astro) | 14.3 |
| | | SF-539 | |
| Lung ca. LX-1 | 15.8 | CNS cancer (astro) | 60.7 |
| | | SNB-75 | |
| Lung ca. NCI-H146 | 4.9 | CNS cancer (glio) | 13.8 |
| | | SNB-19 | |
| Lung ca. SHP-77 | 16.5 | CNS cancer (glio) SF- | 28.5 |
| | | 295 | |
| Lung ca. A549 | 27.2 | Brain (Amygdala) | 5.3 |
| | | Pool | |
| Lung ca. NCI-H526 | 4.1 | Brain (cerebellum) | 5.0 |
| Lung ca. NCI-H23 | 15.0 | Brain (fetal) | 16.4 |
| Lung ca. NCI-H460 | 9.5 | Brain (Hippocampus) | 5.5 |
| | | Pool | |
| Lung ca. HOP-62 | 7.6 | Cerebral Cortex Pool | 8.7 |
| Lung ca. NCI-H522 | 18.2 | Brain (Substantia | 8.3 |
| | | nigra) Pool | |
| Liver | 0.0 | Brain (Thalamus) Pool | 6.3 |
| Fetal Liver | 7.3 | Brain (whole) | 7.0 |
| Liver ca. HepG2 | 29.5 | Spinal Cord Pool | 5.6 |
| Kidney Pool | 17.7 | Adrenal Gland | 6.3 |
| Fetal Kidney | 4.6 | Pituitary gland Pool | 0.8 |
| Renal ca. 786-0 | 17.2 | Salivary Gland | 5.6 |
| Renal ca. A498 | 5.1 | Thyroid (female) | 9.7 |
| Renal ca. ACHN | 17.3 | Pancreatic ca. | 11.7 |
| | | CAPAN2 | |
| Renal ca. UO-31 | 11.1 | Pancreas Pool | 9.2 |

CNS_neurodegeneration_v1.0 Summary: Ag3362 Highest expression of the CG58516-01 gene is seen in the occipital cortex of a control patient and the temporal cortex of an Alzheimer's patient. While the CG58516-01 gene does not appear to be preferentially expressed in Alzheimer's disease, this panel confirms expression of the CG58516-01 gene at moderate/high levels in the brain in an additional set of individuals. Please see Panel 1.4 for discussion of potential utility of this gene in the central nervous system.

General_screening_panel_v1.4 Summary: Ag3362 The CG58516-01 gene is widely expressed in this panel, with highest expression in the breast cancer cell line T47D (CT=29). Significant expression is also seen in cell lines derived from prostate, breast and ovarian cancers. In general, expression of the CG58516-0l gene appears to be greater in the cancer cell lines than in normal tissue. Thus, the expression of this gene could be used to distinguish these cell line types from others in the panel.

Among tissues involved in central nervous system function, this gene is expressed at low but significant levels in all brain regions examined. This gene encodes a protein with a putativie zinc-finger motif. Since these proteins are known to interact with nucleic acids, this suggests that this gene product may play a potential role in transcription. Thus, therapeutic modulation of the CG58516-01 gene product may be used to regulate the transcription of disease-related proteins such as ataxin, huntingtin, or various apoptosis cascade proteins.

Among tissues with metabolic function, this gene is expressed at low levels in pituitary, adipose, adrenal gland, pancreas, thyroid, skeletal muscle, heart, and fetal liver. This widespread expression among these tissues suggests that this gene product may play a role in normal neuroendocrine and metabolic and that disregulated expression of this gene may contribute to neuroendocrine disorders or metabolic diseases, such as obesity and diabetes.

### REFERENCES

1. Zhu W, Chan E K, Li J, Hemmerich P, Tan E M. (2001) Transcription activating property of autoantigen SG2NA and modulating effect of WD-40 repeats. Exp Cell Res. 269(2):312-21

Panel 4D Summary: Ag3362 Results from one experiment with the CG58516-01 gene are not included because the amp plot corresponding to the run indicates that there were problems with the experiment.

F. CG58473-01: Protein Kinase

Expression of gene CG58473-01 was assessed using the primer-probe set Ag3357, described in Table FA. Results of the RTQ-PCR runs are shown in Tables FB and FC.

Table FA. Probe Name Ag3357

TABLE FA
Probe Name Ag3357

| | | | Start | SEQ ID |
|---|---|---|---|---|
| Primers | Sequences | Length | Position | NO: |
| Forward | 5′-gtcaaggtggccctaaaattc-3′ | 21 | 853 | 384 |
| Probe | TET-5′-ccaggacctcatctccaagctgctta-3′-TAMRA | 26 | 897 | 385 |
| Reverse | 5′-agccgttctgaggggttat-3′ | 19 | 926 | 386 |

TABLE FB
General_screening_panel_v1.4

| | Rel. Exp. | | Rel. Exp. |
|---|---|---|---|
| | (%) Ag3357, | | (%) Ag3357, |
| | Run | | Run |
| Tissue Name | 216523477 | Tissue Name | 216523477 |
| Adipose | 0.0 | Renal ca. TK-10 | 13.2 |
| Melanoma* | 0.0 | Bladder | 7.2 |
| Hs688(A).T | | | |
| Melanoma* | 1.1 | Gastric ca. (liver met.) | 5.4 |
| Hs688(B).T | | NCI-N87 | |
| Melanoma* M14 | 50.0 | Gastric ca. KATO III | 49.0 |
| Melanoma* | 33.0 | Colon ca. SW-948 | 14.9 |
| LOXIMVI | | | |
| Melanoma* SK- | 24.7 | Colon ca. SW480 | 95.9 |
| MEL-5 | | | |
| Squamous cell | 11.6 | Colon ca.* (SW480 | 53.6 |
| carcinoma SCC-4 | | met) SW620 | |
| Testis Pool | 8.2 | Colon ca. HT29 | 10.3 |
| Prostate ca.* (bone | 3.2 | Colon ca. HCT-116 | 76.3 |
| met) PC-3 | | | |
| Prostate Pool | 0.0 | Colon ca. CaCo-2 | 14.1 |
| Placenta | 2.4 | Colon cancer tissue | 6.3 |
| Uterus Pool | 0.0 | Colon ca. SW1116 | 18.6 |
| Ovarian ca. | 51.1 | Colon ca. Colo-205 | 24.3 |
| OVCAR-3 | | | |
| Ovarian ca. SK- | 53.2 | Colon ca. SW-48 | 26.1 |
| OV-3 | | | |
| Ovarian ca. | 10.4 | Colon Pool | 4.6 |
| OVCAR-4 | | | |
| Ovarian ca. | 12.3 | Small Intestine Pool | 1.7 |
| OVCAR-5 | | | |
| Ovarian ca. | 10.1 | Stomach Pool | 1.2 |
| IGROV-1 | | | |
| Ovarian ca. | 13.4 | Bone Marrow Pool | 1.0 |
| OVCAR-8 | | | |
| Ovary | 0.0 | Fetal Heart | 0.0 |
| Breast ca. MCF-7 | 20.3 | Heart Pool | 0.0 |
| Breast ca. MDA- | 65.1 | Lymph Node Pool | 1.4 |
| MB-231 | | | |
| Breast ca. BT 549 | 100.0 | Fetal Skeletal Muscle | 0.0 |
| Breast ca. T47D | 34.2 | Skeletal Muscle Pool | 1.6 |
| Breast ca. MDA-N | 36.3 | Spleen Pool | 3.4 |
| Breast Pool | 1.3 | Thymus pool | 4.7 |
| Trachea | 0.0 | CNS cancer | 7.8 |
| | | (glio/astro) U87-MG | |
| Lung | 0.0 | CNS cancer | 54.0 |
| | | (glio/astro) U-118-MG | |
| Fetal Lung | 5.0 | CNS cancer | 7.9 |
| | | (neuro; met) SK-N-AS | |
| Lung ca. NCI-N417 | 17.9 | CNS cancer (astro) | 22.4 |
| | | SF-539 | |
| Lung ca. LX-1 | 28.5 | CNS cancer (astro) | 19.2 |
| | | SNB-75 | |
| Lung ca. NCI-H146 | 74.7 | CNS cancer (glio) | 14.6 |
| | | SNB-19 | |
| Lung ca. SHP-77 | 40.6 | CNS cancer (glio) SF- | 3.0 |
| | | 295 | |
| Lung ca. A549 | 64.6 | Brain (Amygdala) | 0.0 |
| | | Pool | |
| Lung ca. NCI-H526 | 23.8 | Brain (cerebellum) | 0.0 |
| Lung ca. NCI-H23 | 63.7 | Brain (fetal) | 0.0 |
| Lung ca. NCI-H460 | 0.8 | Brain (Hippocampus) | 0.0 |
| | | Pool | |
| Lung ca. HOP-62 | 2.0 | Cerebral Cortex Pool | 0.0 |
| Lung ca. NCI-H522 | 34.4 | Brain (Substantia | 2.6 |
| | | nigra) Pool | |
| Liver | 0.0 | Brain (Thalamus) Pool | 9.3 |
| Fetal Liver | 0.0 | Brain (whole) | 2.5 |
| Liver ca. HepG2 | 11.4 | Spinal Cord Pool | 0.0 |
| Kidney Pool | 0.0 | Adrenal Gland | 0.0 |
| Fetal Kidney | 3.1 | Pituitary gland Pool | 1.4 |
| Renal ca. 786-0 | 20.0 | Salivary Gland | 0.0 |
| Renal ca. A498 | 3.6 | Thyroid (female) | 0.0 |
| Renal ca. ACHN | 18.9 | Pancreatic ca. | 20.4 |
| | | CAPAN2 | |
| Renal ca. UO-31 | 10.4 | Pancreas Pool | 1.3 |

TABLE FC
Panel 4D

| | Rel. Exp. (%) | | Rel. Exp. (%) |
|---|---|---|---|
| | Ag3357, Run | | Ag3357, Run |
| Tissue Name | 165231196 | Tissue Name | 165231196 |
| Secondary Th1 act | 9.0 | HUVEC IL-1beta | 9.5 |
| Secondary Th2 act | 43.2 | HUVEC IFN gamma | 6.3 |
| Secondary Tr1 act | 46.0 | HUVEC TNF alpha + | 7.3 |
| | | IFN gamma | |
| Secondary Th1 rest | 6.7 | HUVEC TNF alpha + | 25.3 |
| | | IL4 | |
| Secondary Th2 rest | 12.2 | HUVEC IL-11 | 13.1 |
| Secondary Tr1 rest | 1.9 | Lung Microvascular EC | 3.3 |
| | | none | |
| Primary Th1 act | 6.1 | Lung Microvascular EC | 7.1 |
| | | TNF alpha + IL-1beta | |
| Primary Th2 act | 21.8 | Microvascular Dermal | 10.9 |
| | | EC none | |
| Primary Tr1 act | 33.0 | Microsvasular Dermal | 7.3 |
| | | EC TNF alpha + IL-1beta | |
| Primary Th1 rest | 28.1 | Bronchial epithelium | 1.9 |
| | | TNF alpha + IL1beta | |
| Primary Th2 rest | 12.1 | Small airway epithelium | 3.6 |
| | | none | |
| Primary Tr1 rest | 29.7 | Small airway epithelium | 36.3 |
| | | TNF alpha + IL-1beta | |
| CD45RA CD4 | 28.5 | Coronery artery SMC rest | 0.0 |
| lymphocyte act | | | |
| CD45RO CD4 | 39.8 | Coronery artery SMC | 0.0 |
| lymphocyte act | | TNF alpha + IL-1beta | |
| CD8 lymphocyte act | 18.6 | Astrocytes rest | 1.4 |
| Secondary CD8 | 26.8 | Astrocytes TNF alpha + | 1.2 |
| lymphocyte rest | | IL-1beta | |
| Secondary CD8 | 19.2 | KU-812 (Basophil) rest | 18.2 |
| lymphocyte act | | | |
| CD4 lymphocyte none | 10.6 | KU-812 (Basophil) | 30.4 |
| | | PMA/ionomycin | |
| 2ry Th1/Th2/Tr1_anti- | 15.6 | CCD1106 | 18.3 |
| CD95 CH11 | | (Keratinocytes) none | |
| LAK cells rest | 0.0 | CCD1106 | 7.7 |
| | | (Keratinocytes) | |
| | | TNF alpha + IL-1beta | |
| LAK cells IL-2 | 42.6 | Liver cirrhosis | 25.7 |
| LAK cells IL-2 + IL-12 | 24.0 | Lupus kidney | 0.0 |
| LAK cells IL-2 + IFN | 24.8 | NCI-H292 none | 7.8 |
| gamma | | | |
| LAK cells IL-2 + IL-18 | 40.3 | NCI-H292 IL-4 | 26.4 |
| LAK cells | 0.0 | NCI-H292 IL-9 | 29.7 |
| PMA/ionomycin | | | |
| NK Cells IL-2 rest | 23.5 | NCI-H292 IL-13 | 20.7 |
| Two Way MLR 3 day | 13.7 | NCI-H292 IFN gamma | 27.9 |
| Two Way MLR 5 day | 13.2 | HPAEC none | 8.6 |
| Two Way MLR 7 day | 11.7 | HPAEC TNF alpha + IL- | 2.4 |
| | | 1beta | |
| PBMC rest | 0.0 | Lung fibroblast none | 5.5 |
| PBMC PWM | 52.1 | Lung fibroblast TNF | 2.2 |
| | | alpha + IL-1beta | |
| PBMC PHA-L | 14.6 | Lung fibroblast IL-4 | 0.0 |
| Ramos (B cell) none | 16.5 | Lung fibroblast IL-9 | 0.0 |
| Ramos (B cell) | 14.7 | Lung fibroblast IL-13 | 0.0 |
| ionomycin | | | |
| B lymphocytes PWM | 100.0 | Lung fibroblast IFN | 0.0 |
| | | gamma | |
| B lymphocytes CD40L | 10.4 | Dermal fibroblast | 40.1 |
| and IL-4 | | CCD1070 rest | |
| EOL-1 dbcAMP | 9.9 | Dermal fibroblast | 43.8 |
| | | CCD1070 TNF alpha | |
| EOL-1 dbcAMP | 13.2 | Dermal fibroblast | 23.5 |
| PMA/ionomycin | | CCD1070 IL-1beta | |
| Dendritic cells none | 4.7 | Dermal fibroblast IFN | 3.7 |
| | | gamma | |
| Dendritic cells LPS | 1.1 | Dermal fibroblast IL-4 | 4.6 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 28.1 |
| Macrophages rest | 4.3 | Lung | 59.0 |
| Macrophages LPS | 0.0 | Thymus | 0.0 |
| HUVEC none | 28.3 | Kidney | 10.0 |
| HUVEC starved | 25.3 | | |

CNS_neurodegeneration_v1.0 Summary: Ag3357—Expression of this gene is low/undetectable (CTs>35) across all of the samples on this panel (data not shown).

General_screening_panel_v1.4 Summary: Ag3357 This gene is primarily expressed in cancer cell lines, with highest expression in a breast cancer cell line BT 549(CT=32.8). This gene is expressed in the following cell lines but not the corresponding healthy tissue: gastric, brain, colon, lung, breast, ovarian cancer and melanomas. Thus, expression of this gene could be used as a diagnostic marker for the presence of these cancers. Furthermore, therapeutic inhibition using antibodies or small molecule drugs might be of use in the treatment of these cancers.

Panel 4D Summary: Ag3357 Highest expression of the CG58473-01 gene is seen in pokeweed mitogen-activated purified peripheral blood B lymphocytes (CT=33.2). In addition, no expression of the transcript is seen in PBMC that contain normal B cells, but the transcript is induced when PBMC are treated with the B cell selective pokeweed mitogen. The transcript is not seen in the B cell lymphoma cell line Ramos regardless of stimulation. Thus, the putative protein encoded by this gene could potentially be used diagnostically to identify activated B cells. Therefore, therapeutics that antagonize the function of this gene product may be useful as therapeutic drugs to reduce or eliminate the symptoms in patients with autoimmune and inflammatory diseases in which B cells play a part in the intiation or progression of the disease process, such as lupus erythematosus, Crohn's disease, ulcerative colitis, multiple sclerosis, chronic obstructive pulmonary disease, asthma, emphysema, rheumatoid arthritis, or psoriasis.

G. CG58470-01: UDP-N-Acetylhexosamine Pyrophosphorylase

Expression of gene CG58470-01 was assessed using the primer-probe set Ag5940, described in Table GA.

Table GA. Probe Name Ag5940

TABLE GA
Probe Name Ag5940

| | | | | SEQ |
|---|---|---|---|---|
| | | | Start | ID |
| Primers | Sequences | Length | Position | NO: |
| Forward | 5′-atatcctgaagctacaacagttagct-3′ | 26 | 422 | 387 |
| Probe | TET-5′-tggcaacaaatgcattattccatattacg-3′-TAMRA | 29 | 459 | 388 |
| Reverse | 5′-gagtgaactcgctggtcatg-3′ | 20 | 489 | 389 |

General_screening_panel_v1.5 Summary: Ag5940—Expression of this gene is low/undetectable (CTs>35) across all of the samples on this panel (data not shown).

Panel 5 Islet Summary: Ag5940—Expression of this gene is low/undetectable (CTs>35) across all of the samples on this panel (data not shown).

H. CG58593-01: Ubiquitin-52

Expression of gene CG58593-01 was assessed using the primer-probe set Ag3421, described in Table HA.

Table HA. Probe Name Ag3421

TABLE HA
Probe Name Ag3421

| | | | Start | SEQ ID |
|---|---|---|---|---|
| Primers | Sequences | Length | Position | NO: |
| Forward | 5′-atctgctgcaagtgctatgc-3′ | 20 | 291 | 390 |
| Probe | TET-5′-cggtgctatcaactgccacaagaaga-3′-TAMRA | 26 | 323 | 391 |
| Reverse | 5′-tgaccttcttcctggggtac-3′ | 20 | 371 | 392 |

CNS_neurodegeneration_v1.0 Summary: Ag3421—Expression of this gene is low/undetectable (CTs>35) across all of the samples on this panel (data not shown).

General_screening_panel_v1.4 Summary: Ag3421—Expression of this gene is low/undetectable (CTs>35) across all of the samples on this panel (data not shown).

Panel 4D Summary: Ag3421—Expression of this gene is low/undetectable (CTs>35) across all of the samples on this panel (data not shown).

I. CG57871-01: Tousled-Like Kinase

Expression of gene CG57871-01 was assessed using the primer-probe set Ag3351, described in Table IA. Results of the RTQ-PCR runs are shown in Tables IB and IC.

Table IA. Probe Name Ag3351

TABLE IA
Probe Name Ag3351

| | | | | |
|---|---|---|---|---|
| | | | Start | SEQ ID |
| Primers | Sequences | Length | Position | NO: |

| | | | | |
|---|---|---|---|---|
| Forward | 5′-gatcctcactgcaacattcttt-3′ | 22 | 346 | 393 |
| Probe | TET-5′-aatcccttaccgcgacgagtagaaca-3′-TAMRA | 26 | 372 | 394 |
| Revers | 5′-gcactgccatctaaaccataga-3′ | 22 | 403 | 395 |

TABLE IB
CNS_neurodegeneration_v1.0

| | Rel. Exp. | | |
|---|---|---|---|
| | (%) Ag3351, | | Rel. Exp.(%) |
| | Run | | Ag3351, Run |
| Tissue Name | 210141594 | Tissue Name | 210141594 |
| AD 1 Hippo | 10.4 | Control (Path) 3 | 3.0 |
| | | Temporal Ctx | |
| AD 2 Hippo | 33.4 | Control (Path) 4 | 65.1 |
| | | Temporal Ctx | |
| AD 3 Hippo | 5.5 | AD 1 Occipital | 20.2 |
| | | Ctx | |
| AD 4 Hippo | 8.4 | AD 2 Occipital | 0.0 |
| | | Ctx (Missing) | |
| AD 5 hippo | 100.0 | AD 3 Occipital | 3.8 |
| | | Ctx | |
| AD 6 Hippo | 33.4 | AD 4 Occipital | 45.1 |
| | | Ctx | |
| Control 2 Hippo | 29.9 | AD 5 Occipital | 15.2 |
| | | Ctx | |
| Control 4 Hippo | 6.7 | AD 6 Occipital | 46.7 |
| | | Ctx | |
| Control (Path) 3 | 3.7 | Control 1 Occipital | 2.7 |
| Hippo | | Ctx | |
| AD 1 Temporal Ctx | 16.8 | Control 2 Occipital | 52.5 |
| | | Ctx | |
| AD 2 Temporal Ctx | 45.1 | Control 3 Occipital | 45.4 |
| | | Ctx | |
| AD 3 Temporal Ctx | 6.9 | Control 4 Occipital | 6.3 |
| | | Ctx | |
| AD 4 Temporal Ctx | 54.0 | Control (Path) 1 | 79.0 |
| | | Occipital Ctx | |
| AD 5 Inf Temporal | 92.0 | Control (Path) 2 | 34.4 |
| Ctx | | Occipital Ctx | |
| AD 5 SupTemporal | 13.0 | Control (Path) 3 | 0.8 |
| Ctx | | Occipital Ctx | |
| AD 6 Inf Temporal | 48.6 | Control (Path) 4 | 40.6 |
| Ctx | | Occipital Ctx | |
| AD 6 Sup Temporal | 56.6 | Control 1 Parietal | 6.9 |
| Ctx | | Ctx | |
| Control 1 Temporal | 6.2 | Control 2 Parietal | 48.0 |
| Ctx | | Ctx | |
| Control 2 Temporal | 29.3 | Control 3 Parietal | 26.1 |
| Ctx | | Ctx | |
| Control 3 Temporal | 32.8 | Control (Path) 1 | 73.7 |
| Ctx | | Parietal Ctx | |
| Control 4 Temporal | 13.9 | Control (Path) 2 | 57.4 |
| Ctx | | Parietal Ctx | |
| Control (Path) 1 | 79.6 | Control (Path) 3 | 3.4 |
| Temporal Ctx | | Parietal Ctx | |
| Control (Path) 2 | 97.3 | Control (Path) 4 | 78.5 |
| Temporal Ctx | | Parietal Ctx | |

TABLE IC
Panel 4D

| | Rel. Exp. (%) | | Rel. Exp. (%) |
|---|---|---|---|
| | Ag3351, Run | | Ag3351, Run |
| Tissue Name | 165222896 | Tissue Name | 165222896 |
| Secondary Th1 act | 16.5 | HUVEC IL-1beta | 15.4 |
| Secondary Th2 act | 26.4 | HUVEC IFN gamma | 13.5 |
| Secondary Tr1 act | 23.3 | HUVEC TNF alpha + | 17.0 |
| | | IFN gamma | |
| Secondary Th1 rest | 6.0 | HUVEC TNF alpha + | 11.0 |
| | | IL4 | |
| Secondary Th2 rest | 10.7 | HUVEC IL-11 | 5.4 |
| Secondary Tr1 rest | 2.1 | Lung Microvascular EC | 12.4 |
| | | none | |
| Primary Th1 act | 19.2 | Lung Microvascular EC | 9.6 |
| | | TNF alpha + IL-1beta | |
| Primary Th2 act | 17.6 | Microvascular Dermal | 14.7 |
| | | EC none | |
| Primary Tr1 act | 36.1 | Microsvasular Dermal | 14.8 |
| | | EC TNF alpha + IL-1beta | |
| Primary Th1 rest | 55.5 | Bronchial epithelium | 14.1 |
| | | TNF alpha + IL1beta | |
| Primary Th2 rest | 43.8 | Small airway epithelium | 7.7 |
| | | none | |
| Primary Tr1 rest | 15.9 | Small airway epithelium | 50.3 |
| | | TNF alpha + IL-1beta | |
| CD45RA CD4 | 13.0 | Coronery artery SMC rest | 15.6 |
| lymphocyte act | | | |
| CD45RO CD4 | 21.0 | Coronery artery SMC | 6.1 |
| lymphocyte act | | TNF alpha + IL-1beta | |
| CD8 lymphocyte act | 12.9 | Astrocytes rest | 11.5 |
| Secondary CD8 | 14.9 | Astrocytes TNF alpha + | 11.8 |
| lymphocyte rest | | IL-1beta | |
| Secondary CD8 | 14.8 | KU-812 (Basophil) rest | 19.2 |
| lymphocyte act | | | |
| CD4 lymphocyte none | 10.7 | KU-812 (Basophil) | 54.0 |
| | | PMA/ionomycin | |
| 2ry Th1/Th2/Tr1_anti- | 12.7 | CCD1106 | 12.2 |
| CD95 CH11 | | (Keratinocytes) none | |
| LAK cells rest | 17.2 | CCD1106 | 9.0 |
| | | (Keratinocytes) | |
| | | TNF alpha + IL-1beta | |
| LAK cells IL-2 | 22.4 | Liver cirrhosis | 7.4 |
| LAK cells IL-2 + IL-12 | 20.4 | Lupus kidney | 3.4 |
| LAK cells IL-2 + IFN | 37.9 | NCI-H292 none | 47.6 |
| gamma | | | |
| LAK cells IL-2 + IL-18 | 18.6 | NCI-H292 IL-4 | 42.3 |
| LAK cells | 10.5 | NCI-H292 IL-9 | 30.4 |
| PMA/ionomycin | | | |
| NK Cells IL-2 rest | 17.8 | NCI-H292 IL-13 | 15.7 |
| Two Way MLR 3 day | 33.2 | NCI-H292 IFN gamma | 25.5 |
| Two Way MLR 5 day | 10.6 | HPAEC none | 13.5 |
| Two Way MLR 7 day | 9.9 | HPAEC TNF alpha + IL- | 17.7 |
| | | 1beta | |
| PBMC rest | 12.8 | Lung fibroblast none | 11.5 |
| PBMC PWM | 63.3 | Lung fibroblast TNF | 12.4 |
| | | alpha + IL-1beta | |
| PBMC PHA-L | 18.0 | Lung fibroblast IL-4 | 31.2 |
| Ramos (B cell) none | 14.0 | Lung fibroblast IL-9 | 22.2 |
| Ramos (B cell) | 77.9 | Lung fibroblast IL-13 | 27.4 |
| ionomycin | | | |
| B lymphocytes PWM | 100.0 | Lung fibroblast IFN | 44.8 |
| | | gamma | |
| B lymphocytes CD40L | 30.8 | Dermal fibroblast | 33.7 |
| and IL-4 | | CCD1070 rest | |
| EOL-1 dbcAMP | 11.3 | Dermal fibroblast | 50.0 |
| | | CCD1070 TNF alpha | |
| EOL-1 dbcAMP | 13.7 | Dermal fibroblast | 13.4 |
| PMA/ionomycin | | CCD1070 IL-1beta | |
| Dendritic cells none | 14.7 | Dermal fibroblast IFN | 14.3 |
| | | gamma | |
| Dendritic cells LPS | 19.8 | Dermal fibroblast IL-4 | 25.7 |
| Dendritic cells anti- | 14.2 | IBD Colitis 2 | 2.0 |
| CD40 | | | |
| Monocytes rest | 22.5 | IBD Crohn's | 3.2 |
| Monocytes LPS | 32.8 | Colon | 26.8 |
| Macrophages rest | 31.0 | Lung | 14.6 |
| Macrophages LPS | 30.8 | Thymus | 28.7 |
| HUVEC none | 18.3 | Kidney | 45.4 |
| HUVEC starved | 45.7 | | |

CNS_neurodegeneration_v1.0 Summary: Ag3351—This panel confirms the expression of this gene at low levels in the brain in an independent group of individuals. While no differential expression of this gene is detected between Alzheimer's diseased postmortem brains and those of non-demented controls, the widespread expression of this gene in the brain suggests that therapeutic modulation of the expression or function of this gene may be effective in the treatment of neurologic disorders such as Parkinson's disease, epilepsy, stroke and multiple sclerosis.

General_screening_panel_v1.4 Summary: Ag3351—Results from one experiment are not included. The amp plot indicates that there were experimental difficulties with this run.

Panel 4D Summary: Ag3351 The CG57871-01 gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

J. CG58590-01 and CG58590-02: PALS Guanylate Kinase

Expression of gene CG58590-01 and CG58590-02 was assessed using the primer-probe set Ag3380, described in Table JA. Results of the RTQ-PCR runs are shown in Tables JB, JC and JD. Please note that CG58590-02 represents a full-length physical clone of the CG58590-01 gene, validating the prediction of the gene sequence.

Table JA. Probe Name Ag3380

TABLE JA
Probe Name Ag3380

| | | | Start | SEQ ID |
|---|---|---|---|---|
| Primers | Sequences | Length | Position | NO: |
| Forward | 5′-tttgatacggcaattgtgaatt-3′ | 22 | 1931 | 396 |
| Probe | TET-5′-ccgatcttgataaagcctatcaggaa-3′-TAMRA | 26 | 1953 | 397 |
| Reverse | 5′-cccactgaggttcagtatcaag-3′ | 22 | 2000 | 398 |

TABLE JB
CNS_neurodegeneration_v1.0

| | | | Rel. Exp. |
|---|---|---|---|
| | Rel. Exp. (%) | | (%) Ag3380, |
| | Ag3380, Run | | Run |
| Tissue Name | 210153753 | Tissue Name | 210153753 |
| AD 1 Hippo | 12.9 | Control (Path) 3 | 4.7 |
| | | Temporal Ctx | |
| AD 2 Hippo | 27.7 | Control (Path) 4 | 24.3 |
| | | Temporal Ctx | |
| AD 3 Hippo | 4.8 | AD 1 Occipital | 15.6 |
| | | Ctx | |
| AD 4 Hippo | 7.7 | AD 2 Occipital | 0.0 |
| | | Ctx (Missing) | |
| AD 5 hippo | 100.0 | AD 3 Occipital | 7.5 |
| | | Ctx | |
| AD 6 Hippo | 64.2 | AD 4 Occipital | 19.1 |
| | | Ctx | |
| Control 2 Hippo | 25.5 | AD 5 Occipital | 29.5 |
| | | Ctx | |
| Control 4 Hippo | 9.9 | AD 6 Occipital | 40.1 |
| | | Ctx | |
| Control (Path) 3 | 8.4 | Control 1 Occipital | 4.2 |
| Hippo | | Ctx | |
| AD 1 Temporal Ctx | 17.6 | Control 2 Occipital | 65.5 |
| | | Ctx | |
| AD 2 Temporal Ctx | 25.3 | Control 3 Occipital | 13.4 |
| | | Ctx | |
| AD 3 Temporal Ctx | 4.9 | Control 4 Occipital | 6.4 |
| | | Ctx | |
| AD 4 Temporal Ctx | 17.4 | Control (Path) 1 | 78.5 |
| | | Occipital Ctx | |
| AD 5 Inf Temporal | 81.8 | Control (Path) 2 | 9.4 |
| Ctx | | Occipital Ctx | |
| AD 5 SupTemporal | 42.9 | Control (Path) 3 | 3.2 |
| Ctx | | Occipital Ctx | |
| AD 6 Inf Temporal | 48.6 | Control (Path) 4 | 9.9 |
| Ctx | | Occipital Ctx | |
| AD 6 Sup Temporal | 53.6 | Control 1 Parietal | 6.0 |
| Ctx | | Ctx | |
| Control 1 Temporal | 5.7 | Control 2 Parietal | 37.1 |
| Ctx | | Ctx | |
| Control 2 Temporal | 34.6 | Control 3 Parietal | 16.5 |
| Ctx | | Ctx | |
| Control 3 Temporal | 10.2 | Control (Path) 1 | 67.4 |
| Ctx | | Parietal Ctx | |
| Control 4 Temporal | 7.1 | Control (Path) 2 | 18.7 |
| Ctx | | Parietal Ctx | |
| Control (Path) 1 | 41.5 | Control (Path) 3 | 3.3 |
| Temporal Ctx | | Parietal Ctx | |
| Control (Path) 2 | 29.5 | Control (Path) 4 | 34.4 |
| Temporal Ctx | | Parietal Ctx | |

TABLE JC
General_screening_panel_v1.4

| | Rel. Exp. | | Rel. Exp. |
|---|---|---|---|
| | (%) Ag3380, | | (%) Ag3380, |
| | Run | | Run |
| Tissue Name | 217043276 | Tissue Name | 217043276 |
| Adipose | 9.0 | Renal ca. TK-10 | 25.5 |
| Melanoma* | 18.9 | Bladder | 15.9 |
| Hs688(A).T | | | |
| Melanoma* | 16.8 | Gastric ca. (liver met.) | 52.5 |
| Hs688(B).T | | NCI-N87 | |
| Melanoma* M14 | 14.9 | Gastric ca. KATO III | 34.6 |
| Melanoma* | 21.6 | Colon ca. SW-948 | 4.9 |
| LOXIMVI | | | |
| Melanoma* SK- | 27.0 | Colon ca. SW480 | 82.4 |
| MEL-5 | | | |
| Squamous cell | 28.7 | Colon ca.* (SW480 | 20.6 |
| carcinoma SCC-4 | | met) SW620 | |
| Testis Pool | 5.1 | Colon ca. HT29 | 9.2 |
| Prostate ca.* (bone | 59.9 | Colon ca. HCT-116 | 20.6 |
| met) PC-3 | | | |
| Prostate Pool | 8.6 | Colon ca. CaCo-2 | 22.8 |
| Placenta | 3.9 | Colon cancer tissue | 10.1 |
| Uterus Pool | 1.9 | Colon ca. SW1116 | 6.2 |
| Ovarian ca. | 32.5 | Colon ca. Colo-205 | 4.9 |
| OVCAR-3 | | | |
| Ovarian ca. SK- | 57.4 | Colon ca. SW-48 | 4.2 |
| OV-3 | | | |
| Ovarian ca. | 14.7 | Colon Pool | 11.4 |
| OVCAR-4 | | | |
| Ovarian ca. | 59.5 | Small Intestine Pool | 9.8 |
| OVCAR-5 | | | |
| Ovarian ca. | 13.1 | Stomach Pool | 7.4 |
| IGROV-1 | | | |
| Ovarian ca. | 19.2 | Bone Marrow Pool | 4.2 |
| OVCAR-8 | | | |
| Ovary | 5.9 | Fetal Heart | 6.3 |
| Breast ca. MCF-7 | 35.1 | Heart Pool | 4.9 |
| Breast ca. MDA- | 58.2 | Lymph Node Pool | 11.4 |
| MB-231 | | | |
| Breast ca. BT 549 | 26.8 | Fetal Skeletal Muscle | 3.3 |
| Breast ca. T47D | 100.0 | Skeletal Muscle Pool | 8.1 |
| Breast ca. MDA-N | 8.7 | Spleen Pool | 5.6 |
| Breast Pool | 10.4 | Thymus Pool | 6.3 |
| Trachea | 5.5 | CNS cancer | 39.2 |
| | | (glio/astro) U87-MG | |
| Lung | 3.8 | CNS cancer | 54.7 |
| | | (glio/astro) U-118-MG | |
| Fetal Lung | 11.8 | CNS cancer | 19.6 |
| | | (neuro; met) SK-N-AS | |
| Lung ca. NCI-N417 | 3.2 | CNS cancer (astro) | 12.2 |
| | | SF-539 | |
| Lung ca. LX-1 | 20.7 | CNS cancer (astro) | 29.7 |
| | | SNB-75 | |
| Lung ca. NCI-H146 | 3.8 | CNS cancer (glio) | 13.4 |
| | | SNB-19 | |
| Lung ca. SHP-77 | 17.9 | CNS cancer (glio) SF- | 28.9 |
| | | 295 | |
| Lung ca. A549 | 30.6 | Brain (Amygdala) | 11.8 |
| | | Pool | |
| Lung ca. NCI-H526 | 3.6 | Brain (cerebellum) | 6.0 |
| Lung ca. NCI-H23 | 29.3 | Brain (fetal) | 8.4 |
| Lung ca. NCI-H460 | 14.8 | Brain (Hippocampus) | 14.5 |
| | | Pool | |
| Lung ca. HOP-62 | 19.5 | Cerebral Cortex Pool | 16.2 |
| Lung ca. NCI-H522 | 28.7 | Brain (Substantia | 16.0 |
| | | nigra) Pool | |
| Liver | 0.4 | Brain (Thalamus) Pool | 22.7 |
| Fetal Liver | 11.9 | Brain (whole) | 5.9 |
| Liver ca. HepG2 | 12.9 | Spinal Cord Pool | 16.0 |
| Kidney Pool | 18.4 | Adrenal Gland | 5.1 |
| Fetal Kidney | 22.8 | Pituitary gland Pool | 3.8 |
| Renal ca. 786-0 | 28.5 | Salivary Gland | 2.1 |
| Renal ca. A498 | 5.0 | Thyroid (female) | 8.2 |
| Renal ca. ACHN | 22.4 | Pancreatic ca. | 51.4 |
| | | CAPAN 2 | |
| Renal ca. UO-31 | 36.9 | Pancreas Pool | 12.3 |

TABLE JD
Panel 4D

| | Rel. Exp. (%) | | Rel. Exp. (%) |
|---|---|---|---|
| | Ag3380, Run | | Ag3380, Run |
| Tissue Name | 165296532 | Tissue Name | 165296532 |
| Secondary Th1 act | 13.1 | HUVEC IL-1beta | 15.0 |
| Secondary Th2 act | 14.6 | HUVEC IFN gamma | 19.6 |
| Secondary Tr1 act | 15.2 | HUVEC TNF alpha + | 28.3 |
| | | IFN gamma | |
| Secondary Th1 rest | 4.6 | HUVEC TNF alpha + | 26.1 |
| | | IL4 | |
| Secondary Th2 rest | 4.7 | HUVEC IL-11 | 7.8 |
| Secondary Tr1 rest | 8.0 | Lung Microvascular EC | 25.5 |
| | | none | |
| Primary Th1 act | 14.9 | Lung Microvascular EC | 19.5 |
| | | TNF alpha + IL-1beta | |
| Primary Th2 act | 13.2 | Microvascular Dermal | 37.9 |
| | | EC none | |
| Primary Tr1 act | 20.7 | Microsvasular Dermal | 24.8 |
| | | EC TNF alpha + IL-1beta | |
| Primary Th1 rest | 35.6 | Bronchial epithelium | 37.1 |
| | | TNF alpha + IL1beta | |
| Primary Th2 rest | 24.0 | Small airway epithelium | 15.0 |
| | | none | |
| Primary Tr1 rest | 16.2 | Small airway epithelium | 100.0 |
| | | TNF alpha + IL-1beta | |
| CD45RA CD4 | 23.3 | Coronery artery SMC rest | 30.1 |
| lymphocyte act | | | |
| CD45RO CD4 | 18.2 | Coronery artery SMC | 13.6 |
| lymphocyte act | | TNF alpha + IL-1beta | |
| CD8 lymphocyte act | 7.4 | Astrocytes rest | 22.5 |
| Secondary CD8 | 13.4 | Astrocytes TNF alpha + | 21.2 |
| lymphocyte rest | | IL-1beta | |
| Secondary CD8 | 4.4 | KU-812 (Basophil) rest | 17.9 |
| lymphocyte act | | | |
| CD4 lymphocyte none | 8.0 | KU-812 (Basophil) | 68.3 |
| | | PMA/ionomycin | |
| 2ry Th1/Th2/Tr1_anti- | 10.7 | CCD1106 | 22.1 |
| CD95 CH11 | | (Keratinocytes) none | |
| LAK cells rest | 13.5 | CCD1106 | 9.2 |
| | | (Keratinocytes) | |
| | | TNF alpha + IL-1beta | |
| LAK cells IL-2 | 12.9 | Liver cirrhosis | 3.1 |
| LAK cells IL-2 + IL-12 | 13.2 | Lupus kidney | 2.9 |
| LAK cells IL-2 + IFN | 15.6 | NCI-H292 none | 48.6 |
| gamma | | | |
| LAK cells IL-2 + IL-18 | 17.0 | NCI-H292 IL-4 | 66.9 |
| LAK cells | 9.5 | NCI-H292 IL-9 | 59.5 |
| PMA/ionomycin | | | |
| NK Cells IL-2 rest | 7.0 | NCI-H292 IL-13 | 36.6 |
| Two Way MLR 3 day | 15.2 | NCI-H292 IFN gamma | 42.6 |
| Two Way MLR 5 day | 7.0 | HPAEC none | 14.3 |
| Two Way MLR 7 day | 9.6 | HPAEC TNF alpha + IL- | 25.9 |
| | | 1beta | |
| PBMC rest | 6.4 | Lung fibroblast none | 12.5 |
| PBMC PWM | 60.7 | Lung fibroblast TNF | 11.0 |
| | | alpha + IL-1beta | |
| PBMC PHA-L | 18.8 | Lung fibroblast IL-4 | 25.9 |
| Ramos (B cell) none | 31.9 | Lung fibroblast IL-9 | 20.6 |
| Ramos (B cell) | 94.0 | Lung fibroblast IL-13 | 18.8 |
| ionomycin | | | |
| B lymphocytes PWM | 42.9 | Lung fibroblast IFN | 23.3 |
| | | gamma | |
| B lymphocytes CD40L | 24.7 | Dermal fibroblast | 59.5 |
| and IL-4 | | CCD1070 rest | |
| EOL-1 dbcAMP | 12.9 | Dermal fibroblast | 64.2 |
| | | CCD1070 TNF alpha | |
| EOL-1 dbcAMP | 10.4 | Dermal fibroblast | 32.8 |
| PMA/ionomycin | | CCD1070 IL-1beta | |
| Dendritic cells none | 19.6 | Dermal fibroblast IFN | 10.7 |
| | | gamma | |
| Dendritic cells LPS | 10.7 | Dermal fibroblast IL-4 | 21.6 |
| Dendritic cells anti- | 18.8 | IBD Colitis 2 | 2.0 |
| CD40 | | | |
| Monocytes rest | 15.0 | IBD Crohn's | 3.6 |
| Monocytes LPS | 13.8 | Colon | 36.9 |
| Macrophages rest | 25.3 | Lung | 19.3 |
| Macrophages LPS | 8.1 | Thymus | 72.2 |
| HUVEC none | 19.9 | Kidney | 24.5 |
| HUVEC starved | 35.8 | | |

CNS_neurodegeneration_v1.0 Summary: Ag3380 This panel does not show differential expression of the CG58590-01 gene in Alzheimer's disease. However, this expression profile confirms the presence of this gene in the brain. Please see Panel 1.3D for discussion of utility of this gene in the central nervous system.

General_screening_panel_v1.4 Summary: Ag3380—This gene is expressed at low to moderate levels in all samples on this pattern. The highest level of expression is seen in breast cancer cell line T47D (CT=27.8). Based on expression in this panel, this gene may be involved in brain, colon, renal, lung, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

This gene product is also expressed in adipose, pancreas, adrenal, thyroid, pituitary, skeletal muscle, heart, and fetal liver. This widespread expression in tissues with metabolic function suggests that this gene product may be important for the pathogenesis, diagnosis, and/or treatment of metabolic and endocrine diseases, including obesity and Types 1 and 2 diabetes;. Furthermore, this gene is more highly expressed in fetal (CT=30.9) liver when compared to expression in the adult (CT>35) and may be useful for the differentiation of the fetal and adult sources of this tissue.

In addition, this gene is expressed at moderate levels in the all regions of the CNS examined. Therefore, this gene may play a role in central nervous system disorders such as Alzheimer's disease, Parkinson's disease, epilepsy, multiple sclerosis, schizophrenia and depression.

Panel 4D Summary: Ag3380—This gene is expressed from moderate to low levels across all of the samples on this panel. The highest expression is seen in small airway epithelium treated with TNFalpha and IL-1beta (CT=28.7). Interestingly, expression is much lower in untreated small airway epithelium (CT=31.5). There is also a significant difference between mononuclear cells treated with PWM (CT=29.5) and untreated cells (CT=32.7). Therefore, expression of this gene can be used to differentiate treated and untreated samples.

Expression of this gene is detected at a moderate level (CT=30.2) in normal colon (similar levels for colon are seen on panel 1.4 (CT=30.9), but is significantly lower in the IBD Colitis 2 (CT=34.4) and IBD Crohn's (CT=33.5) samples. Therefore, therapies designed with the protein encoded for by this gene may potentially modulate colon function and play a role in the identification and treatment of inflammatory or autoimmune diseases, which effect the colon including Crohn's disease and ulcerative colitis.

K. CG58572-01 and CG58572-02: Glucosamine-Phosphate N-Acetyltransferase

Expression of gene CG58572-01 and full length clone CG58572-02 was assessed using the primer-probe set Ag3375, described in Table KA. Results of the RTQ-PCR runs are shown in Tables KB, KC and KD.

Table KA. Probe Name Ag3375

TABLE KA
Probe Name Ag3375

| | | | Start | SEQ ID |
|---|---|---|---|---|
| Primers | Sequences | Length | Position | NO: |
| Forward | 5′-aagaagtggactggagtcagaa-3′ | 22 | 58 | 399 |
| Probe | TET-5′-tacattttctccagccatttccccaa-3′-TAMRA | 26 | 86 | 400 |
| Reverse | 5′-agcagtacaaagaggcctcaa-3′ | 21 | 135 | 401 |

TABLE KB
CNS_neurodegeneration_v1.0

| | Rel. Exp. | | |
|---|---|---|---|
| | (%) Ag3375, | | Rel. Exp. (%) |
| | Run | | Ag3375, Run |
| Tissue Name | 210154239 | Tissue Name | 210154239 |
| AD 1 Hippo | 17.1 | Control (Path) 3 | 4.8 |
| | | Temporal Ctx | |
| AD 2 Hippo | 19.3 | Control (Path) 4 | 27.5 |
| | | Temporal Ctx | |
| AD 3 Hippo | 7.4 | AD 1 Occipital Ctx | 11.5 |
| AD 4 Hippo | 4.5 | AD 2 Occipital Ctx | 0.0 |
| | | (Missing) | |
| AD 5 Hippo | 72.2 | AD 3 Occipital Ctx | 5.9 |
| AD 6 Hippo | 53.6 | AD 4 Occipital Ctx | 12.7 |
| Control 2 Hippo | 20.3 | AD 5 Occipital Ctx | 26.6 |
| Control 4 Hippo | 6.8 | AD 6 Occipital Ctx | 19.8 |
| Control (Path) 3 | 5.5 | Control 1 Occipital | 3.2 |
| Hippo | | Ctx | |
| AD 1 Temporal | 11.6 | Control 2 Occipital | 36.1 |
| Ctx | | Ctx | |
| AD 2 Temporal | 23.8 | Control 3 Occipital | 7.4 |
| Ctx | | Ctx | |
| AD 3 Temporal | 5.5 | Control 4 Occipital | 4.1 |
| Ctx | | Ctx | |
| AD 4 Temporal | 16.5 | Control (Path) 1 | 66.0 |
| Ctx | | Occipital Ctx | |
| AD 5 Inf Temporal | 100.0 | Control (Path) 2 | 8.2 |
| Ctx | | Occipital Ctx | |
| AD 5 Sup | 55.9 | Control (Path) 3 | 1.9 |
| Temporal Ctx | | Occipital Ctx | |
| AD 6 Inf Temporal | 37.9 | Control (Path) 4 | 12.2 |
| Ctx | | Occipital Ctx | |
| AD 6 Sup | 59.5 | Control 1 Parietal | 2.4 |
| Temporal Ctx | | Ctx | |
| Control 1 | 3.5 | Control 2 Parietal | 31.6 |
| Temporal Ctx | | Ctx | |
| Control 2 | 25.3 | Control 3 Parietal | 11.7 |
| Temporal Ctx | | Ctx | |
| Control 3 | 8.2 | Control (Path) 1 | 49.7 |
| Temporal Ctx | | Parietal Ctx | |
| Control 3 | 4.0 | Control (Path) 2 | 15.4 |
| Temporal Ctx | | Parietal Ctx | |
| Control (Path) 1 | 52.9 | Control (Path) 3 | 4.2 |
| Temporal Ctx | | Parietal Ctx | |
| Control (Path) 2 | 26.6 | Control (Path) 4 | 32.5 |
| Temporal Ctx | | Parietal Ctx | |

TABLE KC
Panel 1.3D

| | Rel. Exp. (%) | | Rel. Exp. (%) |
|---|---|---|---|
| | Ag3375, | | Ag3375, |
| Tissue Name | Run 165674233 | Tissue Name | Run 165674233 |
| Liver adenocarcinoma | 51.8 | Kidney (fetal) | 9.7 |
| Pancreas | 9.3 | Renal ca. 786-0 | 19.6 |
| Pancreatic ca. CAPAN 2 | 52.1 | Renal ca. A498 | 26.2 |
| Adrenal gland | 8.9 | Renal ca. RXF 393 | 15.7 |
| Thyroid | 6.3 | Renal ca. ACHN | 8.2 |
| Salivary gland | 18.3 | Renal ca. UO-31 | 35.4 |
| Pituitary gland | 15.1 | Renal ca. TK-10 | 9.8 |
| Brain (fetal) | 15.5 | Liver | 20.4 |
| Brain (whole) | 34.6 | Liver (fetal) | 16.5 |
| Brain (amygdala) | 16.0 | Liver ca. | 49.0 |
| | | (hepatoblast) HepG2 | |
| Brain (cerebellum) | 34.2 | Lung | 4.5 |
| Brain (hippocampus) | 12.1 | Lung (fetal) | 5.4 |
| Brain (substantia nigra) | 12.8 | Lung ca. (small cell) | 32.3 |
| | | LX-1 | |
| Brain (thalamus) | 17.9 | Lung ca. (small cell) | 17.3 |
| | | NCI-H69 | |
| Cerebral Cortex | 10.4 | Lung ca. (s.cell var.) | 30.1 |
| | | SHP-77 | |
| Spinal cord | 13.3 | Lung ca. (large | 66.4 |
| | | cell)NCI-H460 | |
| glio/astro U87-MG | 14.8 | Lung ca. (non-sm. | 19.1 |
| | | cell) A549 | |
| glio/astro U-118-MG | 95.3 | Lung ca. (non-s.cell) | 13.8 |
| | | NCI-H23 | |
| Astrocytoma SW1783 | 42.0 | Lung ca. (non-s.cell) | 18.7 |
| | | HOP-62 | |
| neuro*; met SK-N-AS | 47.0 | Lung ca. (non-s.cl) | 19.5 |
| | | NCI-H522 | |
| Astrocytoma SF-539 | 11.4 | Lung ca. (squam.) | 9.9 |
| | | SW 900 | |
| Astrocytoma SNB-75 | 15.6 | Lung ca. (squam.) | 19.6 |
| | | NCI-H596 | |
| glioma SNB-19 | 11.8 | Mammary gland | 14.6 |
| glioma U251 | 40.9 | Breast ca.* (pl.ef) | 81.2 |
| | | MCF-7 | |
| glioma SF-295 | 10.1 | Breast ca.* (pl.ef) | 91.4 |
| | | MDA-MB-231 | |
| Heart (fetal) | 1.3 | Breast ca.* (pl.ef) | 35.4 |
| | | T47D | |
| Heart | 4.7 | Breast ca. BT-549 | 97.9 |
| Skeletal muscle (fetal) | 1.2 | Breast ca. MDA-N | 14.8 |
| Skeletal muscle | 38.7 | Ovary | 1.6 |
| Bone marrow | 4.6 | Ovarian ca. | 39.2 |
| | | OVCAR-3 | |
| Thymus | 2.7 | Ovarian ca. | 23.0 |
| | | OVCAR-4 | |
| Spleen | 7.9 | Ovarian ca. | 13.8 |
| | | OVCAR-5 | |
| Lymph node | 13.0 | Ovarian ca. | 8.5 |
| | | OVCAR-8 | |
| Colorectal | 3.3 | Ovarian ca. IGROV-1 | 5.6 |
| Stomach | 27.7 | Ovarian ca.* | 44.8 |
| | | (ascites) SK-OV-3 | |
| Small intestine | 19.3 | Uterus | 19.5 |
| Colon ca. SW480 | 16.5 | Placenta | 2.6 |
| Colon ca.* | 29.1 | Prostate | 15.6 |
| SW620(SW480 met) | | | |
| Colon ca. HT29 | 13.8 | Prostate ca.* (bone | 56.6 |
| | | met)PC-3 | |
| Colon ca. HCT-116 | 27.7 | Testis | 40.6 |
| Colon ca. CaCo-2 | 17.4 | Melanoma | 5.5 |
| | | Hs688(A).T | |
| Colon ca. | 26.4 | Melanoma* (met) | 8.9 |
| tissue(ODO3866) | | Hs688(B).T | |
| Colon ca. HCC-2998 | 32.1 | Melanoma UACC- | 17.8 |
| | | 62 | |
| Gastric ca.* (liver met) | 100.0 | Melanoma M14 | 27.7 |
| NCI-N87 | | | |
| Bladder | 28.7 | Melanoma LOX | 6.6 |
| | | IMVI | |
| Trachea | 9.4 | Melanoma* (met) | 13.0 |
| | | SK-MEL-5 | |
| Kidney | 9.0 | Adipose | 8.0 |

TABLE KD
Panel 4D

| | Rel. Exp. (%) | | Rel. Exp. (%) |
|---|---|---|---|
| | Ag3375, Run | | Ag3375, Run |
| Tissue Name | 165296547 | Tissue Name | 165296547 |
| Secondary Th1 act | 14.6 | HUVEC IL-1beta | 24.5 |
| Secondary Th2 act | 13.0 | HUVEC IFN gamma | 24.5 |
| Secondary Tr1 act | 17.3 | HUVEC TNF alpha + | 24.0 |
| | | IFN gamma | |
| Secondary Th1 rest | 0.9 | HUVEC TNF alpha + | 23.2 |
| | | IL4 | |
| Secondary Th2 rest | 1.5 | HUVEC IL-11 | 12.1 |
| Secondary Tr1 rest | 2.9 | Lung Microvascular EC | 21.3 |
| | | none | |
| Primary Th1 act | 16.0 | Lung Microvascular EC | 24.1 |
| | | TNF alpha + IL-1beta | |
| Primary Th2 act | 12.1 | Microvascular Dermal | 27.4 |
| | | EC none | |
| Primary Tr1 act | 25.0 | Microsvasular Dermal | 24.0 |
| | | EC TNF alpha + IL-1beta | |
| Primary Th1 rest | 10.4 | Bronchial epithelium | 20.3 |
| | | TNF alpha + IL1beta | |
| Primary Th2 rest | 6.1 | Small airway epithelium | 11.3 |
| | | none | |
| Primary Tr1 rest | 9.0 | Small airway epithelium | 54.0 |
| | | TNF alpha + IL-1beta | |
| CD45RA CD4 | 14.6 | Coronery artery SMC rest | 23.5 |
| lymphocyte act | | | |
| CD45RO CD4 | 13.6 | Coronery artery SMC | 12.0 |
| lymphocyte act | | TNF alpha + IL-1beta | |
| CD8 lymphocyte act | 14.2 | Astrocytes rest | 5.3 |
| Secondary CD8 | 14.4 | Astrocytes TNF alpha + | 5.4 |
| lymphocyte rest | | IL-1beta | |
| Secondary CD8 | 5.8 | KU-812 (Basophil) rest | 19.5 |
| lymphocyte act | | | |
| CD4 lymphocyte none | 2.4 | KU-812 (Basophil) | 56.3 |
| | | PMA/ionomycin | |
| 2ry Th1/Th2/Tr1_anti- | 2.6 | CCD1106 | 26.6 |
| CD95 CH11 | | (Keratinocytes) none | |
| LAK cells rest | 5.1 | CCD1106 | 7.8 |
| | | (Keratinocytes) | |
| | | TNF alpha + IL-1beta | |
| LAK cells IL-2 | 10.7 | Liver cirrhosis | 2.6 |
| LAK cells IL-2 + IL-12 | 12.5 | Lupus kidney | 0.8 |
| LAK cells IL-2 + IFN | 20.2 | NCI-H292 none | 28.7 |
| gamma | | | |
| LAK cells IL-2 + IL-18 | 16.6 | NCI-H292 IL-4 | 54.7 |
| LAK cells | 12.5 | NCI-H292 IL-9 | 45.7 |
| PMA/ionomycin | | | |
| NK Cells IL-2 rest | 7.1 | NCI-H292 IL-13 | 24.3 |
| Two Way MLR 3 day | 6.8 | NCI-H292 IFN gamma | 33.2 |
| Two Way MLR 5 day | 8.9 | HPAEC none | 17.8 |
| Two Way MLR 7 day | 6.0 | HPAEC TNF alpha + IL- | 30.1 |
| | | 1beta | |
| PBMC rest | 0.8 | Lung fibroblast none | 10.2 |
| PBMC PWM | 42.3 | Lung fibroblast TNF | 6.3 |
| | | alpha + IL-1beta | |
| PBMC PHA-L | 11.6 | Lung fibroblast IL-4 | 27.2 |
| Ramos (B cell) none | 30.6 | Lung fibroblast IL-9 | 26.8 |
| Ramos (B cell) | 100.0 | Lung fibroblast IL-13 | 21.8 |
| ionomycin | | | |
| B lymphocytes PWM | 77.4 | Lung fibroblast IFN | 29.5 |
| | | gamma | |
| B lymphocytes CD40L | 12.2 | Dermal fibroblast | 42.3 |
| and IL-4 | | CCD1070 rest | |
| EOL-1 dbcAMP | 13.0 | Dermal fibroblast | 51.4 |
| | | CCD1070 TNF alpha | |
| EOL-1 dbcAMP | 6.9 | Dermal fibroblast | 22.5 |
| PMA/ionomycin | | CCD1070 IL-1beta | |
| Dendritic cells none | 4.5 | Dermal fibroblast IFN | 11.1 |
| | | gamma | |
| Dendritic cells LPS | 3.8 | Dermal fibroblast IL-4 | 19.5 |
| Dendritic cells anti- | 2.9 | IBD Colitis 2 | 0.7 |
| CD40 | | | |
| Monocytes rest | 2.2 | IBD Crohn's | 0.9 |
| Monocytes LPS | 1.3 | Colon | 7.6 |
| Macrophages rest | 6.6 | Lung | 6.2 |
| Macrophages LPS | 2.7 | Thymus | 9.4 |
| HUVEC none | 17.4 | Kidney | 4.2 |
| HUVEC starved | 37.4 | | |

CNS_neurodegeneration_v1.0 Summary: Ag3375 This panel does not show differential expression of the CG58572-01 gene in Alzheimer's disease. However, this expression profile confirms the presence of this gene in the brain. Please see Panel 1.3D for discussion of utility of this gene in the central nervous system.

Panel 1.3D Summary: Ag3375—This gene is expressed at moderate to low levels in all samples on this panel, with the highest expression in gastric cancer cell line NCI-N87 (CT=28.8). Based on expression in this panel, this gene may be involved in gastric, pancreatic, brain, colon, renal, lung, breast, ovarian and prostate cancer as well as melanomas. Thus, expression of this gene could be used as a diagnostic marker for the presence of these cancers. Furthermore, therapeutic modulation of the expression or function of this gene might be of use in the treatment of these cancers.

This gene product is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

In addition, this gene is expressed at moderate levels in the CNS. Therefore, this gene may play a role in central nervous system disorders such as Alzheimer's disease, Parkinson's disease, epilepsy, multiple sclerosis, schizophrenia and depression.

Panel 4D Summary: Ag3375 The CG58572-01 gene is ubiquitously expressed on this panel, with highest expression in the B cell line Ramos treated with ionomycin (CT=26.2). Significant levels of expression are also seen in pokeweed mitogen-activated B lymphocytes. Therefore, therapies that antagonize the function of this gene product may be useful as therapeutic drugs to reduce or eliminate the symptoms in patients with autoimmune and inflammatory diseases in which B cells play a part in the initiation or progression of the disease process, such as lupus erythematosus, Crohn's disease, ulcerative colitis, multiple sclerosis, chronic obstructive pulmonary disease, asthma, emphysema, rheumatoid arthritis, or psoriasis.

Interestingly, there is a difference between the levels of expression in resting and activated secondary T cells. The level in activated secondary T cells (CT=28.7-29.2) appears to be higher than in resting T cells (CT=31.3-33.1). Therefore, therapeutics designed with the protein encoded by this transcript could be important in the regulation of T cell function.

L. CG58564-01 and CG58564-02: Protein Tyrosine Phosphatase

Expression of gene CG58564-01 and full length clone CG58564-02 was assessed using the primer-probe sets Ag3023 and Ag3373, described in Tables LA and LB. Results of the RTQ-PCR runs are shown in Tables LC, LD, LE and LF.

Table LA. Probe Name Ag3023

TABLE LA
Probe Name Ag3023

| | | | Start | SEQ ID |
|---|---|---|---|---|
| Primers | Sequences | Length | Position | NO: |
| Forward | 5′-ctaatgctggatttgtccatca-3′ | 22 | 492 | 402 |
| Probe | TET-5′-tcaggaatatgaagccatctacctagca-3′-TAMRA | 28 | 517 | 403 |
| Reverse | 5′-tggagtggtgacatcatctgta-3′ | 22 | 555 | 404 |

Table LB. Probe Name Ag3373

TABLE LB
Probe Name Ag3373

| | | | Start | SEQ ID |
|---|---|---|---|---|
| Primers | Sequences | Length | Position | NO: |
| Forward | 5′-atttgtccatcaacttcaggaa-3′ | 22 | 502 | 405 |
| Probe | TET-5′-tgaagccatctacctagcaaaattaaca-3′-TAMRA | 28 | 526 | 406 |
| Reverse | 5′-tggagtggtgacatcatctgta-3′ | 22 | 555 | 407 |

TABLE LC
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3023, Run | Ag3373, Run | Tissue | Ag3023, Run | Ag3373, Run |
| Tissue Name | 209821074 | 210154071 | Name | 209821074 | 210154071 |
| AD 1 Hippo | 10.9 | 16.8 | Control | 9.1 | 8.0 |
| | | | (Path) 3 | | |
| | | | Temporal | | |
| | | | Ctx | | |
| AD 2 Hippo | 34.2 | 37.6 | Control | 40.6 | 65.5 |
| | | | (Path) 4 | | |
| | | | Temporal | | |
| | | | Ctx | | |
| AD 3 Hippo | 12.0 | 15.8 | AD 1 | 24.7 | 29.1 |
| | | | Occipital | | |
| | | | Ctx | | |
| AD 4 Hippo | 13.8 | 10.3 | AD 2 | 0.0 | 0.0 |
| | | | Occipital | | |
| | | | Ctx | | |
| | | | (Missing) | | |
| AD 5 hippo | 60.7 | 57.8 | AD 3 | 14.7 | 15.0 |
| | | | Occipital | | |
| | | | Ctx | | |
| AD 6 Hippo | 80.7 | 72.2 | AD 4 | 35.4 | 22.4 |
| | | | Occipital | | |
| | | | Ctx | | |
| Control 2 | 35.8 | 38.4 | AD 5 | 3.9 | 30.4 |
| Hippo | | | Occipital | | |
| | | | Ctx | | |
| Control 4 | 16.5 | 11.7 | AD 6 | 46.0 | 37.4 |
| Hippo | | | Occipital | | |
| | | | Ctx | | |
| Control (Path) | 13.1 | 15.4 | Control 1 | 9.9 | 10.7 |
| 3 Hippo | | | Occipital | | |
| | | | Ctx | | |
| AD 1 Temporal | 39.0 | 31.4 | Control 2 | 39.0 | 38.4 |
| Ctx | | | Occipital | | |
| | | | Ctx | | |
| AD 2 Temporal | 38.7 | 73.2 | Control 3 | 23.0 | 20.6 |
| Ctx | | | Occipital | | |
| | | | Ctx | | |
| AD 3 Temporal | 9.5 | 13.2 | Control 4 | 13.3 | 13.3 |
| Ctx | | | Occipital | | |
| | | | Ctx | | |
| AD 4 Temporal | 27.9 | 34.9 | Control | 80.1 | 76.3 |
| Ctx | | | (Path) 1 | | |
| | | | Occipital | | |
| | | | Ctx | | |
| AD 5 Inf | 59.0 | 100.0 | Control | 17.3 | 20.0 |
| Temporal Ctx | | | (Path) 2 | | |
| | | | Occipital | | |
| | | | Ctx | | |
| AD 5 | 33.2 | 44.1 | Control | 8.4 | 8.7 |
| SupTemporal | | | (Path) 3 | | |
| Ctx | | | Occipital | | |
| | | | Ctx | | |
| AD 6 Inf | 100.0 | 73.2 | Control | 21.2 | 20.6 |
| Temporal Ctx | | | (Path) 4 | | |
| | | | Occipital | | |
| | | | Ctx | | |
| AD 6 Sup | 79.6 | 80.1 | Control 1 | 12.1 | 16.3 |
| Temporal Ctx | | | Parietal Ctx | | |
| Control 1 | 10.2 | 13.7 | Control 2 | 48.0 | 40.9 |
| Temporal Ctx | | | Parietal Ctx | | |
| Control 2 | 41.2 | 31.9 | Control 3 | 17.9 | 16.3 |
| Temporal Ctx | | | Parietal Ctx | | |
| Control 3 | 20.3 | 20.0 | Control | 74.7 | 64.2 |
| Temporal Ctx | | | (Path) 1 | | |
| | | | Parietal Ctx | | |
| Control 4 | 9.7 | 9.9 | Control | 28.9 | 59.9 |
| Temporal Ctx | | | (Path) 2 | | |
| | | | Parietal Ctx | | |
| Control (Path) | 59.9 | 68.3 | Control | 10.2 | 9.0 |
| 1 Temporal Ctx | | | (Path) 3 | | |
| | | | Parietal Ctx | | |
| Control (Path) | 40.3 | 41.2 | Control | 44.8 | 43.8 |
| 2 Temporal Ctx | | | (Path) 4 | | |
| | | | Parietal Ctx | | |

TABLE LD
General_screening_panel_v1.4

| | Rel. | | Rel. |
|---|---|---|---|
| | Exp. (%) | | Exp. (%) |
| | Ag3373, | | Ag3373, |
| | Run | | Run |
| Tissue Name | 217043119 | Tissue Name | 217043119 |
| Adipose | 12.0 | Renal ca. TK-10 | 20.3 |
| Melanoma* | 30.8 | Bladder | 23.2 |
| Hs688(A).T | | | |
| Melanoma* | 69.3 | Gastric ca. (liver met.) | 25.3 |
| Hs688(B).T | | NCI-N87 | |
| Melanoma* M14 | 15.0 | Gastric ca. KATO III | 30.8 |
| Melanoma* | 26.6 | Colon ca. SW-948 | 9.7 |
| LOXIMVI | | | |
| Melanoma* SK- | 21.5 | Colon ca. SW480 | 35.1 |
| MEL-5 | | | |
| Squamous cell | 33.0 | Colon ca.* (SW480 | 13.9 |
| carcinoma SCC-4 | | met) SW620 | |
| Testis Pool | 19.8 | Colon ca. HT29 | 8.5 |
| Prostate ca.* (bone | 100.0 | Colon ca. HCT-116 | 36.9 |
| met) PC-3 | | | |
| Prostate Pool | 9.2 | Colon ca. CaCo-2 | 42.9 |
| Placenta | 3.8 | Colon cancer tissue | 9.0 |
| Uterus Pool | 7.4 | Colon ca. SW1116 | 5.8 |
| Ovarian ca. | 28.5 | Colon ca. Colo-205 | 4.3 |
| OVCAR-3 | | | |
| Ovarian ca. SK- | 40.3 | Colon ca. SW-48 | 4.2 |
| OV-3 | | | |
| Ovarian ca. | 20.0 | Colon Pool | 20.7 |
| OVCAR-4 | | | |
| Ovarian ca. | 35.1 | Small Intestine Pool | 12.2 |
| OVCAR-5 | | | |
| Ovarian ca. | 10.9 | Stomach Pool | 9.9 |
| IGROV-1 | | | |
| Ovarian ca. | 9.2 | Bone Marrow Pool | 11.6 |
| OVCAR-8 | | | |
| Ovary | 9.7 | Fetal Heart | 20.7 |
| Breast ca. MCF-7 | 37.6 | Heart Pool | 10.6 |
| Breast ca. MDA- | 37.1 | Lymph Node Pool | 17.9 |
| MB-231 | | | |
| Breast ca. BT 549 | 62.4 | Fetal Skeletal Muscle | 12.3 |
| Breast ca. T47D | 61.1 | Skeletal Muscle Pool | 16.0 |
| Breast ca. MDA-N | 10.0 | Spleen Pool | 11.6 |
| Breast Pool | 17.3 | Thymus Pool | 12.2 |
| Trachea | 12.0 | CNS cancer | 29.1 |
| | | (glio/astro) U87-MG | |
| Lung | 6.7 | CNS cancer | 69.3 |
| | | (glio/astro) U-118-MG | |
| Fetal Lung | 34.2 | CNS cancer | 34.9 |
| | | (neuro; met) SK-N-AS | |
| Lung ca. NCI-N417 | 5.4 | CNS cancer (astro) | 19.1 |
| | | SF-539 | |
| Lung ca. LX-1 | 17.2 | CNS cancer (astro) | 35.8 |
| | | SNB-75 | |
| Lung ca. NCI-H146 | 3.0 | CNS cancer (glio) | 11.3 |
| | | SNB-19 | |
| Lung ca. SHP-77 | 18.6 | CNS cancer (glio) SF- | 26.4 |
| | | 295 | |
| Lung ca. A549 | 29.1 | Brain (Amygdala) | 4.5 |
| | | Pool | |
| Lung ca. NCI-H526 | 4.6 | Brain (cerebellum) | 8.1 |
| Lung ca. NCI-H23 | 31.6 | Brain (fetal) | 13.2 |
| Lung ca. NCI-H460 | 18.2 | Brain (Hippocampus) | 5.3 |
| | | Pool | |
| Lung ca. HOP-62 | 14.1 | Cerebral Cortex Pool | 5.4 |
| Lung ca. NCI-H522 | 31.6 | Brain (Substantia | 4.8 |
| | | nigra) Pool | |
| Liver | 1.2 | Brain (Thalamus) Pool | 8.0 |
| Fetal Liver | 32.3 | Brain (whole) | 6.2 |
| Liver ca. HepG2 | 14.6 | Spinal Cord Pool | 6.6 |
| Kidney Pool | 22.1 | Adrenal Gland | 8.1 |
| Fetal Kidney | 26.1 | Pituitary gland Pool | 3.0 |
| Renal ca. 786-0 | 28.7 | Salivary Gland | 4.7 |
| Renal ca. A498 | 11.3 | Thyroid (female) | 4.4 |
| Renal ca. ACHN | 12.2 | Pancreatic ca. | 17.3 |
| | | CAPAN2 | |
| Renal ca. UO-31 | 24.1 | Pancreas Pool | 17.1 |

TABLE LE
Panel 1.3D

| | Rel. Exp. (%) | | Rel. Exp. (%) |
|---|---|---|---|
| | Ag3023, Run | | Ag3023, Run |
| Tissue Name | 167966931 | Tissue Name | 167966931 |
| Liver adenocarcinoma | 51.1 | Kidney (fetal) | 26.2 |
| Pancreas | 6.1 | Renal ca. 786-0 | 34.2 |
| Pancreatic ca. CAPAN 2 | 17.7 | Renal ca. A498 | 17.6 |
| Adrenal gland | 3.8 | Renal ca. RXF 393 | 17.2 |
| Thyroid | 3.0 | Renal ca. ACHN | 13.5 |
| Salivary gland | 3.9 | Renal ca. UO-31 | 0.0 |
| Pituitary gland | 3.6 | Renal ca. TK-10 | 23.0 |
| Brain (fetal) | 8.1 | Liver | 11.7 |
| Brain (whole) | 8.5 | Liver (fetal) | 8.0 |
| Brain (amygdala) | 6.7 | Liver ca. | 26.2 |
| | | (hepatoblast) HepG2 | |
| Brain (cerebellum) | 15.2 | Lung | 3.1 |
| Brain (hippocampus) | 5.4 | Lung (fetal) | 11.0 |
| Brain (substantia nigra) | 9.0 | Lung ca. (small cell) | 12.9 |
| | | LX-1 | |
| Brain (thalamus) | 4.2 | Lung ca. (small cell) | 9.9 |
| | | NCI-H69 | |
| Cerebral Cortex | 2.0 | Lung ca. (s.cell var.) | 67.8 |
| | | SHP-77 | |
| Spinal cord | 6.9 | Lung ca. (large | 3.4 |
| | | cell)NCI-H460 | |
| glio/astro U87-MG | 28.5 | Lung ca. (non-sm. | 45.1 |
| | | cell) A549 | |
| glio/astro U-118-MG | 46.7 | Lung ca. (non-s.cell) | 22.7 |
| | | NCI-H23 | |
| astrocytoma SW1783 | 40.6 | Lung ca. (non-s.cell) | 25.7 |
| | | HOP-62 | |
| neuro*; met SK-N-AS | 27.2 | Lung ca. (non-s.cl) | 38.2 |
| | | NCI-H522 | |
| astrocytoma SF-539 | 29.7 | Lung ca. (squam.) | 27.4 |
| | | SW 900 | |
| astrocytoma SNB-75 | 35.1 | Lung ca. (squam.) | 29.9 |
| | | NCI-H596 | |
| glioma SNB-19 | 15.6 | Mammary gland | 5.1 |
| glioma U251 | 37.9 | Breast ca.* (pl.ef) | 47.0 |
| | | MCF-7 | |
| glioma SF-295 | 18.4 | Breast ca.* (pl.ef) | 22.7 |
| | | MDA-MB-231 | |
| Heart (fetal) | 2.9 | Breast ca.* (pl.ef) | 86.5 |
| | | T47D | |
| Heart | 12.9 | Breast ca. BT-549 | 15.9 |
| Skeletal muscle (fetal) | 3.4 | Breast ca. MDA-N | 10.4 |
| Skeletal muscle | 36.3 | Ovary | 2.9 |
| Bone marrow | 4.5 | Ovarian ca. | 26.1 |
| | | OVCAR-3 | |
| Thymus | 14.3 | Ovarian ca. | 16.3 |
| | | OVCAR-4 | |
| Spleen | 8.7 | Ovarian ca. | 83.5 |
| | | OVCAR-5 | |
| Lymph node | 11.8 | Ovarian ca. | 9.3 |
| | | OVCAR-8 | |
| Colorectal | 10.4 | Ovarian ca. IGROV-1 | 12.0 |
| Stomach | 7.8 | Ovarian ca.* | 100.0 |
| | | (ascites) SK-OV-3 | |
| Small intestine | 5.1 | Uterus | 4.9 |
| Colon ca. SW480 | 19.3 | Placenta | 1.3 |
| Colon ca.* | 42.9 | Prostate | 3.9 |
| SW620(SW480 met) | | | |
| Colon ca. HT29 | 9.9 | Prostate ca.* (bone | 78.5 |
| | | met)PC-3 | |
| Colon ca. HCT-116 | 26.2 | Testis | 9.7 |
| Colon ca. CaCo-2 | 41.5 | Melanoma | 5.9 |
| | | Hs688(A).T | |
| Colon ca. | 6.3 | Melanoma* (met) | 14.2 |
| tissue(ODO3866) | | Hs688(B).T | |
| Colon ca. HCC-2998 | 16.0 | Melanoma UACC- | 14.0 |
| | | 62 | |
| Gastric ca.* (liver met) | 18.8 | Melanoma M14 | 5.7 |
| NCI-N87 | | | |
| Bladder | 30.6 | Melanoma LOX | 8.8 |
| | | IMVI | |
| Trachea | 3.2 | Melanoma* (met) | 14.7 |
| | | SK-MEL-5 | |
| Kidney | 9.6 | Adipose | 18.9 |

TABLE LF
Panel 4D

| | Rel. | Rel. | | Rel. | Rel. |
|---|---|---|---|---|---|
| | Exp. (%) | Exp. (%) | | Exp. (%) | Exp. (%) |
| | Ag3023, | Ag3373, | | Ag3023, | Ag3373, |
| | Run | Run | | Run | Run |
| Tissue Name | 164516146 | 165296617 | Tissue Name | 164516146 | 165296617 |
| Secondary Th1 act | 18.6 | 17.9 | HUVEC IL-1beta | 20.3 | 18.6 |
| Secondary Th2 act | 24.3 | 28.5 | HUVEC IFN | 25.3 | 22.7 |
| | | | gamma | | |
| Secondary Tr1 act | 22.8 | 21.8 | HUVEC TNF | 16.3 | 18.0 |
| | | | alpha + IFN | | |
| | | | gamma | | |
| Secondary Th1 rest | 7.5 | 6.8 | HUVEC TNF | 18.2 | 13.4 |
| | | | alpha + IL4 | | |
| Secondary Th2 rest | 11.6 | 9.5 | HUVEC IL-11 | 13.7 | 9.9 |
| Secondary Tr1 rest | 12.1 | 10.7 | Lung | 25.7 | 21.6 |
| | | | Microvascular EC | | |
| | | | none | | |
| Primary Th1 act | 20.7 | 16.5 | Lung | 26.2 | 18.3 |
| | | | Microvascular EC | | |
| | | | TNF alpha + IL- | | |
| | | | 1beta | | |
| Primary Th2 act | 20.2 | 19.3 | Microvascular | 27.5 | 21.3 |
| | | | Dermal EC none | | |
| Primary Tr1 act | 23.3 | 27.7 | Microsvasular | 20.7 | 19.9 |
| | | | Dermal EC | | |
| | | | TNF alpha + IL- | | |
| | | | 1beta | | |
| Primary Th1 rest | 51.1 | 51.4 | Bronchial | 13.0 | 16.3 |
| | | | epithelium | | |
| | | | TNF alpha + | | |
| | | | IL1beta | | |
| Primary Th2 rest | 26.2 | 29.5 | Small airway | 8.1 | 8.5 |
| | | | epithelium none | | |
| Primary Tr1 rest | 23.7 | 26.1 | Small airway | 50.3 | 39.8 |
| | | | epithelium | | |
| | | | TNF alpha + IL- | | |
| | | | 1beta | | |
| CD45RA CD4 | 14.6 | 11.0 | Coronery artery | 20.2 | 18.9 |
| lymphocyte act | | | SMC rest | | |
| CD45RO CD4 | 25.2 | 22.4 | Coronery artery | 12.0 | 9.8 |
| lymphocyte act | | | SMC TNF alpha + | | |
| | | | IL-1beta | | |
| CD8 lymphocyte | 20.4 | 15.8 | Astrocytes rest | 10.4 | 11.1 |
| act | | | | | |
| Secondary CD8 | 16.5 | 19.9 | Astrocytes | 11.7 | 9.8 |
| lymphocyte rest | | | TNF alpha + IL- | | |
| | | | 1beta | | |
| Secondary CD8 | 13.2 | 9.3 | KU-812 | 47.6 | 38.2 |
| lymphocyte act | | | (Basophil) rest | | |
| CD4 lymphocyte | 17.1 | 11.6 | KU-812 | 94.0 | 92.0 |
| none | | | (Basophil) | | |
| | | | PMA/ionomycin | | |
| 2ry | 18.3 | 16.6 | CCD1106 | 19.9 | 13.2 |
| Th1/Th2/Tr1_anti- | | | (Keratinocytes) | | |
| CD95 CH11 | | | none | | |
| LAK cells rest | 25.5 | 16.0 | CCD1106 | 6.0 | 4.8 |
| | | | (Keratinocytes) | | |
| | | | TNF alpha + IL- | | |
| | | | 1beta | | |
| LAK cells IL-2 | 27.2 | 22.5 | Liver cirrhosis | 3.1 | 2.7 |
| LAK cells IL- | 27.2 | 19.3 | Lupus kidney | 2.1 | 1.7 |
| 2 + IL-12 | | | | | |
| LAK cells IL- | 36.3 | 34.4 | NCI-H292 none | 30.1 | 18.9 |
| 2 + IFN gamma | | | | | |
| LAK cells IL-2 + | 35.1 | 29.7 | NCI-H292 IL-4 | 33.9 | 34.6 |
| IL-18 | | | | | |
| LAK cells | 12.4 | 11.0 | NCI-H292 IL-9 | 40.1 | 29.1 |
| PMA/ionomycin | | | | | |
| NK Cells IL-2 rest | 20.0 | 15.0 | NCI-H292 IL-13 | 16.2 | 14.2 |
| Two Way MLR 3 | 24.0 | 16.7 | NCI-H292 IFN | 16.6 | 18.4 |
| day | | | gamma | | |
| Two Way MLR 5 | 12.9 | 10.1 | HPAEC none | 13.6 | 13.5 |
| day | | | | | |
| Two Way MLR 7 | 11.4 | 9.5 | HPAEC TNF | 25.3 | 25.3 |
| day | | | alpha + IL-1beta | | |
| PBMC rest | 13.7 | 10.5 | Lung fibroblast | 11.4 | 14.2 |
| | | | none | | |
| PBMC PWM | 69.3 | 66.4 | Lung fibroblast | 6.1 | 7.2 |
| | | | TNF alpha + IL- | | |
| | | | 1beta | | |
| PBMC PHA-L | 22.8 | 17.7 | Lung fibroblast | 28.5 | 29.1 |
| | | | IL-4 | | |
| Ramos (B cell) | 24.1 | 19.3 | Lung fibroblast | 23.0 | 23.3 |
| none | | | IL-9 | | |
| Ramos (B cell) | 100.0 | 100.0 | Lung fibroblast | 20.6 | 18.9 |
| ionomycin | | | IL-13 | | |
| B lymphocytes | 71.7 | 74.2 | Lung fibroblast | 39.0 | 32.5 |
| PWM | | | IFN gamma | | |
| B lymphocytes | 29.1 | 28.7 | Dermal fibroblast | 33.9 | 31.0 |
| CD40L and IL-4 | | | CCD1070 rest | | |
| EOL-1 dbcAMP | 12.1 | 10.5 | Dermal fibroblast | 76.8 | 62.0 |
| | | | CCD1070 TNF | | |
| | | | alpha | | |
| EOL-1 dbcAMP | 14.5 | 10.9 | Dermal fibroblast | 20.3 | 13.9 |
| PMA/ionomycin | | | CCD1070 IL- | | |
| | | | 1beta | | |
| Dendritic cells | 13.2 | 14.8 | Dermal fibroblast | 14.2 | 9.5 |
| none | | | IFN gamma | | |
| Dendritic cells LPS | 11.7 | 8.3 | Dermal fibroblast | 26.4 | 20.4 |
| | | | IL-4 | | |
| Dendritic cells | 17.7 | 12.7 | IBD Colitis 2 | 2.6 | 2.2 |
| anti-CD40 | | | | | |
| Monocytes rest | 16.7 | 17.6 | IBD Crohn's | 2.0 | 1.9 |
| Monocytes LPS | 6.4 | 5.0 | Colon | 11.9 | 10.5 |
| Macrophages rest | 23.5 | 22.8 | Lung | 13.3 | 11.2 |
| Macrophages LPS | 9.9 | 7.1 | Thymus | 14.4 | 12.9 |
| HUVEC none | 20.6 | 17.9 | Kidney | 27.5 | 19.6 |
| HUVEC starved | 43.5 | 38.4 | | | |

CNS_neurodegeneration_v1.0 Summary: Ag3023/Ag3373 This panel does not show differential expression of the CG58564-01 gene in Alzheimer's disease. However, this expression profile confirms the presence of this gene in the brain. Please see Panel 1.3D for discussion of utility of this gene in the central nervous system.

General_screening_panel_v1.4 Summary: Ag3373 Highest expression of the CG58564-01 gene is seen in a prostate cancer cell line (CT=27). Overall, this gene is expressed at moderate levels in the cancer cell lines in this panel. A higher level of expression is observed in clusters of cell lines derived from prostate, brain, melanoma, colon, lung, breast and ovarian cancer when compared to expression in normal prostate, brain, colon, lung, breast and ovary. Thus, this gene could potentially be used as a diagnostic marker of cancer in these tissues. Furthermore, inhibition of the activity of this gene product using small molecule drugs may be effective in the treatment of cancer in these tissues.

Among tissues with metabolic function, this gene product has moderate levels of expression in adipose, heart, skeletal muscle, adrenal, pituitary, thyroid and pancreas. Thus, this gene product may be a small molecule target for the treatment of endocrine and metabolic diseases, including obesity and Types 1 and 2 diabetes.

In addition, this gene appears to be differentially expressed in fetal (CT value=29) vs adult liver (CT value=33) and may be useful for differentiation between the two sources of this tissue.

This gene is also expressed at moderate levels in all central nervous system samples present on this panel. Please see Panel 10.3D for discussion of utility of this gene in the central nervous system.

Panel 1.3D Summary: Ag3023 The CG58564-01 gene is ubiquitously expressed among the samples on this panel, with highest expression in an ovarian cancer cell line (CT=28.8). Overall, the expression of this gene shows good agreement with panel 1.4. A higher level of expression is observed in prostate, brain, melanoma, colon, lung, pancreatic, breast and ovarian cancer cell lines than the normal prostate, brain, colon, lung, pancreas, breast and ovary. Thus, expression of this gene could be used as a diagnostic marker of cancer in these tissues. Furthermore, inhibition of the activity of this gene product using small molecule drugs may be effective in the treatment of cancer in these tissues.

Among tissues with metabolic function, expression of this gene is widespread, as in the previous panel. Please see Panel 1.4 for discussion of utility of this gene in metabolic disease.

This gene represents a phosphatase that is also expressed at low to moderate levels across the CNS. Some phosphatases comprise a family of MAP kinase regulating enzymes, members of which are upregulated in brains subjected to insults such as ischemia and seizure activity. MAP kinases are kown to regulate neurotrophic and neurotoxic pathways. Consequently, agents that modulate the activity of this gene may have utility in attenuating the apoptotic and neurodegenerative processes following brain insults.

### REFERENCES

1. Wiessner C. The dual specificity phosphatase PAC-1 is transcriptionally induced in the rat brain following transient forebrain ischemia. Brain Res Mol Brain Res February 1995;28(2):353-6

2. Boschert U, Muda M, Camps M, Dickinson R, Arkinstall S. Induction of the dual specificity phosphatase PAC1 in rat brain following seizure activity. Neuroreport Sep. 29, 1997;8(14):3077-80

Panel 4D Summary: Ag3023/Ag3373 The CG585864-01 gene is expressed at high to moderate levels in a wide range of cell types and tissues of significance in the immune response in health and disease. Highest expression of this gene is seen in ionomycin treated Ramos B cells (CT=26.83). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

M. CG58564-03: Dual Specificity Phosphatase

Expression of gene CG58564-03 was assessed using the primer-probe sets Ag3023, Ag3373 and Ag5847, described in Tables MA, MB and MC. Results of the RTQ-PCR runs are shown in Tables MD, ME, MF, MG and NM.

Table MA. Probe Name Ag3023

TABLE MA
Probe Name Ag3023

| | | | Start | SEQ ID |
|---|---|---|---|---|
| Primers | Sequences | Length | Position | NO: |
| Forward | 5′-ctaatgctggatttgtccatca-3′ | 22 | 261 | 408 |
| Probe | TET-5′-tcaggaatatgaagccatctacctagca-3′-TAMRA | 28 | 230 | 409 |
| Reverse | 5′-tggagtggtgacatcatctgta-3′ | 22 | 198 | 410 |

Table MB. Probe Name Ag3373

TABLE MB
Probe Name Ag3373

| | | | Start | SEQ ID |
|---|---|---|---|---|
| Primers | Sequences | Length | Position | NO: |
| Forward | 5′-atttgtccatcaacttcaggaa-3′ | 22 | 251 | 411 |
| Probe | TET-5′-tgaagccatctacctagcaaaattaaca-3′-TAMRA | 28 | 221 | 412 |
| Reverse | 5′-tggagtggtgacatcatctgta-3′ | 22 | 198 | 413 |

Table MC. Probe Name Ag5847

TABLE MC
Probe Name Ag5847

| | | | Start | SEQ ID |
|---|---|---|---|---|
| Primers | Sequences | Length | Position | NO: |
| Forward | 5′-cattccaaatgtttctgtagt-3′ | 21 | 335 | 414 |
| Probe | TET-5′-ttcatagcagatgaatatgggcctaagaac-3′-TAMRA | 30 | 371 | 415 |
| Reverse | 5′-ccacagtgcaaggaagac-3′ | 18 | 457 | 416 |

TABLE MD
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3023, Run | Ag3373, Run | Tissue | Ag3023, Run | Ag3373, Run |
| Tissue Name | 209821074 | 210154071 | Name | 209821074 | 210154071 |
| AD 1 Hippo | 10.9 | 16.8 | Control | 9.1 | 8.0 |
| | | | (Path) 3 | | |
| | | | Temporal | | |
| | | | Ctx | | |
| AD 2 Hippo | 34.2 | 37.6 | Control | 40.6 | 65.5 |
| | | | (Path) 4 | | |
| | | | Temporal | | |
| | | | Ctx | | |
| AD 3 Hippo | 12.0 | 15.8 | AD 1 | 24.7 | 29.1 |
| | | | Occipital | | |
| | | | Ctx | | |
| AD 4 Hippo | 13.8 | 10.3 | AD 2 | 0.0 | 0.0 |
| | | | Occipital | | |
| | | | Ctx | | |
| | | | (Missing) | | |
| AD 5 hippo | 60.7 | 57.8 | AD 3 | 14.7 | 15.0 |
| | | | Occipital | | |
| | | | Ctx | | |
| AD 6 Hippo | 80.7 | 72.2 | AD 4 | 35.4 | 22.4 |
| | | | Occipital | | |
| | | | Ctx | | |
| Control 2 | 35.8 | 38.4 | AD 5 | 3.9 | 30.4 |
| Hippo | | | Occipital | | |
| | | | Ctx | | |
| Control 4 | 16.5 | 11.7 | AD 6 | 46.0 | 37.4 |
| Hippo | | | Occipital | | |
| | | | Ctx | | |
| Control (Path) | 13.1 | 15.4 | Control 1 | 9.9 | 10.7 |
| 3 Hippo | | | Occipital | | |
| | | | Ctx | | |
| AD 1 Temporal | 39.0 | 31.4 | Control 2 | 39.0 | 38.4 |
| Ctx | | | Occipital | | |
| | | | Ctx | | |
| AD 2 Temporal | 38.7 | 73.2 | Control 3 | 23.0 | 20.6 |
| Ctx | | | Occipital | | |
| | | | Ctx | | |
| AD 3 Temporal | 9.5 | 13.2 | Control 4 | 13.3 | 13.3 |
| Ctx | | | Occipital | | |
| | | | Ctx | | |
| AD 4 Temporal | 27.9 | 34.9 | Control | 80.1 | 76.3 |
| Ctx | | | (Path) 1 | | |
| | | | Occipital | | |
| | | | Ctx | | |
| AD 5 Inf | 59.0 | 100.0 | Control | 17.3 | 20.0 |
| Temporal Ctx | | | (Path) 2 | | |
| | | | Occipital | | |
| | | | Ctx | | |
| AD 5 | 33.2 | 44.1 | Control | 8.4 | 8.7 |
| SupTemporal | | | (Path) 3 | | |
| Ctx | | | Occipital | | |
| | | | Ctx | | |
| AD 6 Inf | 100.0 | 73.2 | Control | 21.2 | 20.6 |
| Temporal Ctx | | | (Path) 4 | | |
| | | | Occipital | | |
| | | | Ctx | | |
| AD 6 Sup | 79.6 | 80.1 | Control 1 | 12.1 | 16.3 |
| Temporal Ctx | | | Parietal Ctx | | |
| Control 1 | 10.2 | 13.7 | Control 2 | 48.0 | 40.9 |
| Temporal Ctx | | | Parietal Ctx | | |
| Control 2 | 41.2 | 31.9 | Control 3 | 17.9 | 16.3 |
| Temporal Ctx | | | Parietal Ctx | | |
| Control 3 | 20.3 | 20.0 | Control | 74.7 | 64.2 |
| Temporal Ctx | | | (Path) 1 | | |
| | | | Parietal Ctx | | |
| Control 4 | 9.7 | 9.9 | Control | 28.9 | 59.9 |
| Temporal Ctx | | | (Path) 2 | | |
| | | | Parietal Ctx | | |
| Control (Path) | 59.9 | 68.3 | Control | 10.2 | 9.0 |
| 1 Temporal Ctx | | | (Path) 3 | | |
| | | | Parietal Ctx | | |
| Control (Path) | 40.3 | 41.2 | Control | 44.8 | 43.8 |
| 2 Temporal Ctx | | | (Path) 4 | | |
| | | | Parietal Ctx | | |

TABLE ME
General_screening_panel_v1.4

| | Rel. Exp. (%) | | Rel. Exp. (%) |
|---|---|---|---|
| | Ag3373, Run | | Ag3373, Run |
| Tissue Name | 217043119 | Tissue Name | 217043119 |
| Adipose | 12.0 | Renal ca. TK-10 | 20.3 |
| Melanoma* | 30.8 | Bladder | 23.2 |
| Hs688(A).T | | | |
| Melanoma* | 69.3 | Gastric ca. (liver met.) | 25.3 |
| Hs688(B).T | | NCI-N87 | |
| Melanoma* M14 | 15.0 | Gastric ca. KATO III | 30.8 |
| Melanoma* | 26.6 | Colon ca. SW-948 | 9.7 |
| LOXIMVI | | | |
| Melanoma* SK- | 21.5 | Colon ca. SW480 | 35.1 |
| MEL-5 | | | |
| Squamous cell | 33.0 | Colon ca.* (SW480 | 13.9 |
| carcinoma SCC-4 | | met) SW620 | |
| Testis Pool | 19.8 | Colon ca. HT29 | 8.5 |
| Prostate ca.* (bone | 100.0 | Colon ca. HCT-116 | 36.9 |
| met) PC-3 | | | |
| Prostate Pool | 9.2 | Colon ca. CaCo-2 | 42.9 |
| Placenta | 3.8 | Colon cancer tissue | 9.0 |
| Uterus Pool | 7.4 | Colon ca. SW1116 | 5.8 |
| Ovarian ca. | 28.5 | Colon ca. Colo-205 | 4.3 |
| OVCAR-3 | | | |
| Ovarian ca. SK- | 40.3 | Colon ca. SW-48 | 4.2 |
| OV-3 | | | |
| Ovarian ca. | 20.0 | Colon Pool | 20.7 |
| OVCAR-4 | | | |
| Ovarian ca. | 35.1 | Small Intestine Pool | 12.2 |
| OVCAR-5 | | | |
| Ovarian ca. | 10.9 | Stomach Pool | 9.9 |
| IGROV-1 | | | |
| Ovarian ca. | 9.2 | Bone Marrow Pool | 11.6 |
| OVCAR-8 | | | |
| Ovary | 9.7 | Fetal Heart | 20.7 |
| Breast ca. MCF-7 | 37.6 | Heart Pool | 10.6 |
| Breast ca. MDA- | 37.1 | Lymph Node Pool | 17.9 |
| MB-231 | | | |
| Breast ca. BT 549 | 62.4 | Fetal Skeletal Muscle | 12.3 |
| Breast ca. T47D | 61.1 | Skeletal Muscle Pool | 16.0 |
| Breast ca. MDA-N | 10.0 | Spleen Pool | 11.6 |
| Breast Pool | 17.3 | Thymus Pool | 12.2 |
| Trachea | 12.0 | CNS cancer | 29.1 |
| | | (glio/astro) U87-MG | |
| Lung | 6.7 | CNS cancer | 69.3 |
| | | (glio/astro) U-118-MG | |
| Fetal Lung | 34.2 | CNS cancer | 34.9 |
| | | (neuro; met) SK-N-AS | |
| Lung ca. NCI-N417 | 5.4 | CNS cancer (astro) SF- | 19.1 |
| | | 539 | |
| Lung ca. LX-1 | 17.2 | CNS cancer (astro) | 35.8 |
| | | SNB-75 | |
| Lung ca. NCI-H146 | 3.0 | CNS cancer (glio) | 11.3 |
| | | SNB-19 | |
| Lung ca. SHP-77 | 18.6 | CNS cancer (glio) SF- | 26.4 |
| | | 295 | |
| Lung ca. A549 | 29.1 | Brain (Amygdala) Pool | 4.5 |
| Lung ca. NCI-H526 | 4.6 | Brain (cerebellum) | 8.1 |
| Lung ca. NCI-H23 | 31.6 | Brain (fetal) | 13.2 |
| Lung ca. NCI-H460 | 18.2 | Brain (Hippocampus) | 5.3 |
| | | Pool | |
| Lung ca. HOP-62 | 14.1 | Cerebral Cortex Pool | 5.4 |
| Lung ca. NCI-H522 | 31.6 | Brain (Substantia | 4.8 |
| | | nigra) Pool | |
| Liver | 1.2 | Brain (Thalamus) Pool | 8.0 |
| Fetal Liver | 32.3 | Brain (whole) | 6.2 |
| Liver ca. HepG2 | 14.6 | Spinal Cord Pool | 6.6 |
| Kidney Pool | 22.1 | Adrenal Gland | 8.1 |
| Fetal Kidney | 26.1 | Pituitary gland Pool | 3.0 |
| Renal ca. 786-0 | 28.7 | Salivary Gland | 4.7 |
| Renal ca. A498 | 11.3 | Thyroid (female) | 4.4 |
| Renal ca. ACHN | 12.2 | Pancreatic ca. | 17.3 |
| | | CAPAN2 | |
| Renal ca. UO-31 | 24.1 | Pancreas Pool | 17.1 |

TABLE MF
General_screening_panel_v1.5

| | Rel. Exp. (%) | | Rel. Exp. (%) |
|---|---|---|---|
| | Ag5847, Run | | Ag5847, Run |
| Tissue Name | 247590257 | Tissue Name | 247590257 |
| Adipose | 0.1 | Renal ca. TK-10 | 0.2 |
| Melanoma* | 0.1 | Bladder | 0.1 |
| Hs688(A).T | | | |
| Melanoma* | 0.1 | Gastric ca. (liver met.) | 0.2 |
| Hs688(B).T | | NCI-N87 | |
| Melanoma* M14 | 0.1 | Gastric ca. KATO III | 0.1 |
| Melanoma* | 0.1 | Colon ca. SW-948 | 0.1 |
| LOXIMVI | | | |
| Melanoma* SK- | 0.1 | Colon ca. SW480 | 0.2 |
| MEL-5 | | | |
| Squamous cell | 0.2 | Colon ca.* (SW480 | 1.8 |
| carcinoma SCC-4 | | met) SW620 | |
| Testis Pool | 0.1 | Colon ca. HT29 | 0.0 |
| Prostate ca.* (bone | 0.6 | Colon ca. HCT-116 | 0.2 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.2 | Colon ca. Colo-205 | 0.0 |
| OVCAR-3 | | | |
| Ovarian ca. SK- | 0.1 | Colon ca. SW-48 | 0.0 |
| OV-3 | | | |
| Ovarian ca. | 0.1 | Colon Pool | 0.1 |
| OVCAR-4 | | | |
| Ovarian ca. | 0.2 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.1 | Bone Marrow Pool | 0.0 |
| OVCAR-8 | | | |
| Ovary | 0.1 | Fetal Heart | 0.1 |
| Breast ca. MCF-7 | 0.3 | Heart Pool | 0.0 |
| Breast ca. MDA- | 0.2 | Lymph Node Pool | 0.1 |
| MB-231 | | | |
| Breast ca. BT 549 | 0.2 | Fetal Skeletal Muscle | 0.1 |
| Breast ca. T47D | 0.2 | Skeletal Muscle Pool | 0.1 |
| Breast ca. MDA-N | 0.1 | Spleen Pool | 0.1 |
| Breast Pool | 0.0 | Thymus Pool | 0.1 |
| Trachea | 0.1 | CNS cancer | 0.2 |
| | | (glio/astro) U87-MG | |
| Lung | 0.0 | CNS cancer | 0.5 |
| | | (glio/astro) U-118-MG | |
| Fetal Lung | 0.2 | CNS cancer | 0.2 |
| | | (neuro; met) SK-N-AS | |
| Lung ca. NCI-N417 | 0.0 | CNS cancer (astro) SF- | 0.1 |
| | | 539 | |
| Lung ca. LX-1 | 0.0 | CNS cancer (astro) | 0.2 |
| | | SNB-75 | |
| Lung ca. NCI-H146 | 0.0 | CNS cancer (glio) | 0.1 |
| | | SNB-19 | |
| Lung ca. SHP-77 | 0.1 | CNS cancer (glio) SF- | 0.2 |
| | | 295 | |
| Lung ca. A549 | 0.2 | Brain (Amygdala) Pool | 0.0 |
| Lung ca. NCI-H526 | 0.0 | Brain (cerebellum) | 0.0 |
| Lung ca. NCI-H23 | 0.1 | Brain (fetal) | 0.1 |
| Lung ca. NCI-H460 | 0.1 | Brain (Hippocampus) | 0.0 |
| | | Pool | |
| Lung ca. HOP-62 | 0.0 | Cerebral Cortex Pool | 0.0 |
| Lung ca. NCI-H522 | 0.1 | Brain (Substantia | 0.0 |
| | | nigra) Pool | |
| Liver | 0.0 | Brain (Thalamus) Pool | 0.0 |
| Fetal Liver | 0.1 | Brain (whole) | 0.0 |
| Liver ca. HepG2 | 0.1 | Spinal Cord Pool | 0.0 |
| Kidney Pool | 0.1 | Adrenal Gland | 0.0 |
| Fetal Kidney | 0.1 | Pituitary gland Pool | 0.0 |
| Renal ca. 786-0 | 0.2 | Salivary Gland | 100.0 |
| Renal ca. A498 | 0.1 | Thyroid (female) | 0.0 |
| Renal ca. ACHN | 0.1 | Pancreatic ca. | 0.1 |
| | | CAPAN2 | |
| Renal ca. UO-31 | 0.1 | Pancreas Pool | 0.0 |

TABLE MG
Panel 1.3D

| | Rel. Exp. (%) | | Rel. Exp. (%) |
|---|---|---|---|
| | Ag3023, Run | | Ag3023, Run |
| Tissue Name | 167966931 | Tissue Name | 167966931 |
| Liver adenocarcinoma | 51.1 | Kidney (fetal) | 26.2 |
| Pancreas | 6.1 | Renal ca. 786-0 | 34.2 |
| Pancreatic ca. CAPAN 2 | 17.7 | Renal ca. A498 | 17.6 |
| Adrenal gland | 3.8 | Renal ca. RXF 393 | 17.2 |
| Thyroid | 3.0 | Renal ca. ACHN | 13.5 |
| Salivary gland | 3.9 | Renal ca. UO-31 | 0.0 |
| Pituitary gland | 3.6 | Renal ca. TK-10 | 23.0 |
| Brain (fetal) | 8.1 | Liver | 11.7 |
| Brain (whole) | 8.5 | Liver (fetal) | 8.0 |
| Brain (amygdala) | 6.7 | Liver ca. | 26.2 |
| | | (hepatoblast) HepG2 | |
| Brain (cerebellum) | 15.2 | Lung | 3.1 |
| Brain (hippocampus) | 5.4 | Lung (fetal) | 11.0 |
| Brain (substantia nigra) | 9.0 | Lung ca. (small cell) | 12.9 |
| | | LX-1 | |
| Brain (thalamus) | 4.2 | Lung ca. (small cell) | 9.9 |
| | | NCI-H69 | |
| Cerebral Cortex | 2.0 | Lung ca. (s.cell var.) | 67.8 |
| | | SHP-77 | |
| Spinal cord | 6.9 | Lung ca. (large | 3.4 |
| | | cell)NCI-H460 | |
| Glio/astro U87-MG | 28.5 | Lung ca. (non-sm. | 45.1 |
| | | cell) A549 | |
| Glio/astro U-118-MG | 46.7 | Lung ca. (non-s.cell) | 22.7 |
| | | NCI-H23 | |
| astrocytoma SW1783 | 40.6 | Lung ca. (non-s.cell) | 25.7 |
| | | HOP-62 | |
| neuro*; met SK-N-AS | 27.2 | Lung ca. (non-s.cl) | 38.2 |
| | | NCI-H522 | |
| astrocytoma SF-539 | 29.7 | Lung ca. (squam.) | 27.4 |
| | | SW 900 | |
| astrocytoma SNB-75 | 35.1 | Lung ca. (squam.) | 29.9 |
| | | NCI-H596 | |
| glioma SNB-19 | 15.6 | Mammary gland | 5.1 |
| glioma U251 | 37.9 | Breast ca.* (pl.ef) | 47.0 |
| | | MCF-7 | |
| glioma SF-295 | 18.4 | Breast ca.* (pl.ef) | 22.7 |
| | | MDA-MB-231 | |
| Heart (fetal) | 2.9 | Breast ca.* (pl.ef) | 86.5 |
| | | T47D | |
| Heart | 12.9 | Breast ca. BT-549 | 15.9 |
| Skeletal muscle (fetal) | 3.4 | Breast ca. MDA-N | 10.4 |
| Skeletal muscle | 36.3 | Ovary | 2.9 |
| Bone marrow | 4.5 | Ovarian ca. | 26.1 |
| | | OVCAR-3 | |
| Thymus | 14.3 | Ovarian ca. | 16.3 |
| | | OVCAR-4 | |
| Spleen | 8.7 | Ovarian ca. | 83.5 |
| | | OVCAR-5 | |
| Lymph node | 11.8 | Ovarian ca. | 9.3 |
| | | OVCAR-8 | |
| Colorectal | 10.4 | Ovarian ca. IGROV-1 | 12.0 |
| Stomach | 7.8 | Ovarian ca.* | 100.0 |
| | | (ascites) SK-OV-3 | |
| Small intestine | 5.1 | Uterus | 4.9 |
| Colon ca. SW480 | 19.3 | Placenta | 1.3 |
| Colon ca.* | 42.9 | Prostate | 3.9 |
| SW620(SW480 met) | | | |
| Colon ca. HT29 | 9.9 | Prostate ca.* (bone | 78.5 |
| | | met)PC-3 | |
| Colon ca. HCT-116 | 26.2 | Testis | 9.7 |
| Colon ca. CaCo-2 | 41.5 | Melanoma | 5.9 |
| | | Hs688(A).T | |
| Colon ca. | 6.3 | Melanoma* (met) | 14.2 |
| tissue(ODO3866) | | Hs688(B).T | |
| Colon ca. HCC-2998 | 16.0 | Melanoma UACC- | 14.0 |
| | | 62 | |
| Gastric ca.* (liver met) | 18.8 | Melanoma M14 | 5.7 |
| NCI-N87 | | | |
| Bladder | 30.6 | Melanoma LOX | 8.8 |
| | | IMVI | |
| Trachea | 3.2 | Melanoma* (met) | 14.7 |
| | | SK-MEL-5 | |
| Kidney | 9.6 | Adipose | 18.9 |

TABLE MH
Panel 4D

| | Rel. | Rel. | | Rel. | Rel. |
|---|---|---|---|---|---|
| | Exp. (%) | Exp. (%) | | Exp. (%) | Exp. (%) |
| | Ag3023, | Ag3373, | | Ag3023, | Ag3373, |
| | Run | Run | | Run | Run |
| Tissue Name | 164516146 | 165296617 | Tissue Name | 164516146 | 165296617 |
| Secondary Th1 act | 18.6 | 17.9 | HUVEC IL-1beta | 20.3 | 18.6 |
| Secondary Th2 act | 24.3 | 28.5 | HUVEC IFN | 25.3 | 22.7 |
| | | | gamma | | |
| Secondary Tr1 act | 22.8 | 21.8 | HUVEC TNF | 16.3 | 18.0 |
| | | | alpha + IFN | | |
| | | | gamma | | |
| Secondary Th1 rest | 7.5 | 6.8 | HUVEC TNF | 18.2 | 13.4 |
| | | | alpha + IL4 | | |
| Secondary Th2 rest | 11.6 | 9.5 | HUVEC IL-11 | 13.7 | 9.9 |
| Secondary Tr1 rest | 12.1 | 10.7 | Lung | 25.7 | 21.6 |
| | | | Microvascular EC | | |
| | | | none | | |
| Primary Th1 act | 20.7 | 16.5 | Lung | 26.2 | 18.3 |
| | | | Microvascular EC | | |
| | | | TNF alpha + IL- | | |
| | | | 1beta | | |
| Primary Th2 act | 20.2 | 19.3 | Microvascular | 27.5 | 21.3 |
| | | | Dermal EC none | | |
| Primary Tr1 act | 23.3 | 27.7 | Microsvasular | 20.7 | 19.9 |
| | | | Dermal EC | | |
| | | | TNF alpha + IL- | | |
| | | | 1beta | | |
| Primary Th1 rest | 51.1 | 51.4 | Bronchial | 13.0 | 16.3 |
| | | | epithelium | | |
| | | | TNF alpha + | | |
| | | | IL1beta | | |
| Primary Th2 rest | 26.2 | 29.5 | Small airway | 8.1 | 8.5 |
| | | | epithelium none | | |
| Primary Tr1 rest | 23.7 | 26.1 | Small airway | 50.3 | 39.8 |
| | | | epithelium | | |
| | | | TNF alpha + IL- | | |
| | | | 1beta | | |
| CD45RA CD4 | 14.6 | 11.0 | Coronery artery | 20.2 | 18.9 |
| lymphocyte act | | | SMC rest | | |
| CD45RO CD4 | 25.2 | 22.4 | Coronery artery | 12.0 | 9.8 |
| lymphocyte act | | | SMC TNF alpha + | | |
| | | | IL-1beta | | |
| CD8 lymphocyte | 20.4 | 15.8 | Astrocytes rest | 10.4 | 11.1 |
| act | | | | | |
| Secondary CD8 | 16.5 | 19.9 | Astrocytes | 11.7 | 9.8 |
| lymphocyte rest | | | TNF alpha + IL- | | |
| | | | 1beta | | |
| Secondary CD8 | 13.2 | 9.3 | KU-812 | 47.6 | 38.2 |
| lymphocyte act | | | (Basophil) rest | | |
| CD4 lymphocyte | 17.1 | 11.6 | KU-812 | 94.0 | 92.0 |
| none | | | (Basophil) | | |
| | | | PMA/ionomycin | | |
| 2ry | 18.3 | 16.6 | CCD1106 | 19.9 | 13.2 |
| Th1/Th2/Tr1_anti- | | | (Keratinocytes) | | |
| CD95 CH11 | | | none | | |
| LAK cells rest | 25.5 | 16.0 | CCD1106 | 6.0 | 4.8 |
| | | | (Keratinocytes) | | |
| | | | TNF alpha + IL- | | |
| | | | 1beta | | |
| LAK cells IL-2 | 27.2 | 22.5 | Liver cirrhosis | 3.1 | 2.7 |
| LAK cells IL- | 27.2 | 19.3 | Lupus kidney | 2.1 | 1.7 |
| 2 + IL-12 | | | | | |
| LAK cells IL- | 36.3 | 34.4 | NCI-H292 none | 30.1 | 18.9 |
| 2 + IFN gamma | | | | | |
| LAK cells IL-2 + | 35.1 | 29.7 | NCI-H292 IL-4 | 33.9 | 34.6 |
| IL-18 | | | | | |
| LAK cells | 12.4 | 11.0 | NCI-H292 IL-9 | 40.1 | 29.1 |
| PMA/ionomycin | | | | | |
| NK Cells IL-2 rest | 20.0 | 15.0 | NCI-H292 IL-13 | 16.2 | 14.2 |
| Two Way MLR 3 | 24.0 | 16.7 | NCI-H292 IFN | 16.6 | 18.4 |
| day | | | gamma | | |
| Two Way MLR 5 | 12.9 | 10.1 | HPAEC none | 13.6 | 13.5 |
| day | | | | | |
| Two Way MLR 7 | 11.4 | 9.5 | HPAEC TNF | 25.3 | 25.3 |
| day | | | alpha + IL-1beta | | |
| PBMC rest | 13.7 | 10.5 | Lung fibroblast | 11.4 | 14.2 |
| | | | none | | |
| PBMC PWM | 69.3 | 66.4 | Lung fibroblast | 6.1 | 7.2 |
| | | | TNF alpha + IL- | | |
| | | | 1beta | | |
| PBMC PHA-L | 22.8 | 17.7 | Lung fibroblast | 28.5 | 29.1 |
| | | | IL-4 | | |
| Ramos (B cell) | 24.1 | 19.3 | Lung fibroblast | 23.0 | 23.3 |
| none | | | IL-9 | | |
| Ramos (B cell) | 100.0 | 100.0 | Lung fibroblast | 20.6 | 18.9 |
| ionomycin | | | IL-13 | | |
| B lymphocytes | 71.7 | 74.2 | Lung fibroblast | 39.0 | 32.5 |
| PWM | | | IFN gamma | | |
| B lymphocytes | 29.1 | 28.7 | Dermal fibroblast | 33.9 | 31.0 |
| CD40L and IL-4 | | | CCD1070 rest | | |
| EOL-1 dbcAMP | 12.1 | 10.5 | Dermal fibroblast | 76.8 | 62.0 |
| | | | CCD1070 TNF | | |
| | | | alpha | | |
| EOL-1 dbcAMP | 14.5 | 10.9 | Dermal fibroblast | 20.3 | 13.9 |
| PMA/ionomycin | | | CCD1070 IL- | | |
| | | | 1beta | | |
| Dendritic cells | 13.2 | 14.8 | Dermal fibroblast | 14.2 | 9.5 |
| none | | | IFN gamma | | |
| Dendritic cells LPS | 11.7 | 8.3 | Dermal fibroblast | 26.4 | 20.4 |
| | | | IL-4 | | |
| Dendritic cells | 17.7 | 12.7 | IBD Colitis 2 | 2.6 | 2.2 |
| anti-CD40 | | | | | |
| Monocytes rest | 16.7 | 17.6 | IBD Crohn's | 2.0 | 1.9 |
| Monocytes LPS | 6.4 | 5.0 | Colon | 11.9 | 10.5 |
| Macrophages rest | 23.5 | 22.8 | Lung | 13.3 | 11.2 |
| Macrophages LPS | 9.9 | 7.1 | Thymus | 14.4 | 12.9 |
| HUVEC none | 20.6 | 17.9 | Kidney | 27.5 | 19.6 |
| HUVEC starved | 43.5 | 38.4 | | | |

CNS_neurodegeneration_v1.0 Summary: Ag3023/Ag3373 This panel does not show differential expression of the CG56804-03 gene, a splice variant of CG56804-01, in Alzheimrer's disease. However, this expression profile confirms the presence of this gene in the brain. Please see Panel 1.3D for discussion of utility of this gene in the central nervous system. Ag5847—This primer pair recognizes only the splice variant CG58564-03. Expression of this variant is low/undetectable (CTs>35) across all of the samples on this panel (data not shown).

General_screening_panel_v1.4 Summary: Ag3373 Highest expression of the CG56804-03 gene is seen in a prostate cancer cell line (CT=27). Overall, this gene is expressed at moderate levels in the cancer cell lines in this panel. A higher level of expression is observed in clusters of cell lines derived from prostate, brain, melanoma, colon, lung, breast and ovarian cancer when compared to expression in normal prostate, brain, colon, lung, breast and ovary. Thus, this gene could potentially be used as a diagnostic marker of cancer in these tissues. Furthermore, inhibition of the activity of this gene product using small molecule drugs may be effective in the treatment of cancer in these tissues.

Among tissues with metabolic function, this gene product has moderate levels of expression in adipose, heart, skeletal muscle, adrenal, pituitary, thyroid and pancreas. Thus, this gene product may be a small molecule target for the treatment of endocrine and metabolic diseases, including obesity and Types 1 and 2 diabetes.

In addition, this gene appears to be differentially expressed in fetal (CT value=29) vs adult liver (CT value=33) and may be useful for differentiation between the two sources of this tissue.

This gene is also expressed at moderate levels in all central nervous system samples present on this panel. Please see Panel 1.3D for discussion of utility of this gene in the central nervous system.

General_screening_panel_v1.5 Summary: Ag5847—This primer pair, specific to this splice variant, CG58564-03. Expression of this variant is highest in salivary gland (CT=28.6). Therefore, expression of this gene can be used to differentiate this sample from others on the panel.

Panel 1.3D Summary: Ag3023 The CG56804-03 gene is ubiquitously expressed among the samples on this panel, with highest expression in an ovarian cancer cell line (CT=28.8). Overall, the expression of this gene shows good agreement with panel 1.4. A higher level of expression is observed in prostate, brain, melanoma, colon, lung, pancreatic, breast and ovarian cancer cell lines than the normal prostate, brain, colon, lung, pancreas, breast and ovary. Thus, expression of this gene could be used as a diagnostic marker of cancer in these tissues. Furthermore, inhibition of the activity of this gene product using small molecule drugs may be effective in the treatment of cancer in these tissues.

Among tissues with metabolic function, expression of this gene is widespread, as in the previous panel. Please see Panel 1.4 for discussion of utility of this gene in metabolic disease.

This gene represents a dual specificity phosphatase that is also expressed at low to moderate levels across the CNS. Dual-specificity phosphatases comprise a family of MAP kinase regulating enzymes, members of which are upregulated in brains subjected to insults such as ischemia and seizure activity. MAP kinases are kown to regulate neurotrophic and neurotoxic pathways. Consequently, agents that modulate the activity of this gene may have utility in attenuating the apoptotic and neurodegenerative processes following brain insults.

Panel 4.1D Summary: Ag5847—This primer pair recognizes a splice variant of CG58564-03. Expression of this variant is low/undetectable (CTs>35) across all of the samples on this panel (data not shown).

Panel 4D Summary: Ag3023/Ag3373 The CG56804-03 gene is expressed at high to moderate levels in a wide range of cell types and tissues of significance in the immune response in health and disease. Highest expression of this gene is seen in ionomycin treated Ramos 13 cells (CT=26.83). Therefore, targeting of this gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

N. CG58564-04: Dual Specificity Phosphatase

Expression of gene CG58564-04, a splice variant of CG58564-01, was assessed using the primer-probe sets Ag3023, Ag3373 and Ag5844, described in Tables NA, NB and NC. Results of the RTQ-PCR runs are shown in Tables ND, NE, NF and NG.

Table NA. Probe Name Ag3023

TABLE NA
Probe Name Ag3023

| | | | Start | SEQ ID |
|---|---|---|---|---|
| Primers | Sequences | Length | Position | NO: |
| Forward | 5′-ctaatgctggatttgtccatca-3′ | 22 | 190 | 417 |
| Probe | TET-5′-tcaggaatatgaagccatctacctagca-3′-TAMRA | 28 | 159 | 418 |
| Reverse | 5′-tggagtggtgacatcatctgta-3′ | 22 | 127 | 419 |

Table NB. Probe Name Ag3373

TABLE NB
Probe Name Ag3373

| | | | Start | SEQ ID |
|---|---|---|---|---|
| Primers | Sequences | Length | Position | NO: |
| Forward | 5′-atttgtccatcaacttcaggaa-3′ | 22 | 180 | 420 |
| Probe | TET-5′-tgaagccatctacctagcaaaattaaca-3′-TAMRA | 28 | 150 | 421 |
| Reverse | 5′-tggagtggtgacatcatctgta-3′ | 22 | 127 | 422 |

Table NC. Probe Name Ag5844

TABLE NC
Probe Name Ag5844

| | | | Start | SEQ ID |
|---|---|---|---|---|
| Primers | Sequences | Length | Position | NO: |
| Forward | 5′-ccttagtctaaataactgctg-3′ | 21 | 377 | 423 |
| Probe | TET-5′-agtttgcttcaatattttgtcgtatgcata-3′-TAMRA | 30 | 415 | 424 |
| Reverse | 5′-aggagtggacctaccctat-3′ | 19 | 552 | 425 |

TABLE ND
CNS_neurodegeneration_v1.0

| | Rel. Exp. (%) | Rel. Exp. (%) | | Rel. Exp. (%) | Rel. Exp. (%) |
|---|---|---|---|---|---|
| | Ag3023, Run | Ag3373, Run | Tissue | Ag3023, Run | Ag3373, Run |
| Tissue Name | 209821074 | 210154071 | Name | 209821074 | 210154071 |
| AD 1 Hippo | 10.9 | 16.8 | Control | 9.1 | 8.0 |
| | | | (Path) 3 | | |
| | | | Temporal | | |
| | | | Ctx | | |
| AD 2 Hippo | 34.2 | 37.6 | Control | 40.6 | 65.5 |
| | | | (Path) 4 | | |
| | | | Temporal | | |
| | | | Ctx | | |
| AD 3 Hippo | 12.0 | 15.8 | AD 1 | 24.7 | 29.1 |
| | | | Occipital | | |
| | | | Ctx | | |
| AD 4 Hippo | 13.8 | 10.3 | AD 2 | 0.0 | 0.0 |
| | | | Occipital | | |
| | | | Ctx | | |
| | | | (Missing) | | |
| AD 5 hippo | 60.7 | 57.8 | AD 3 | 14.7 | 15.0 |
| | | | Occipital | | |
| | | | Ctx | | |
| AD 6 Hippo | 80.7 | 72.2 | AD 4 | 35.4 | 22.4 |
| | | | Occipital | | |
| | | | Ctx | | |
| Control 2 | 35.8 | 38.4 | AD 5 | 3.9 | 30.4 |
| Hippo | | | Occipital | | |
| | | | Ctx | | |
| Control 4 | 16.5 | 11.7 | AD 6 | 46.0 | 37.4 |
| Hippo | | | Occipital | | |
| | | | Ctx | | |
| Control (Path) | 13.1 | 15.4 | Control 1 | 9.9 | 10.7 |
| 3 Hippo | | | Occipital | | |
| | | | Ctx | | |
| AD 1 Temporal | 39.0 | 31.4 | Control 2 | 39.0 | 38.4 |
| Ctx | | | Occipital | | |
| | | | Ctx | | |
| AD 2 Temporal | 38.7 | 73.2 | Control 3 | 23.0 | 20.6 |
| Ctx | | | Occipital | | |
| | | | Ctx | | |
| AD 3 Temporal | 9.5 | 13.2 | Control 4 | 13.3 | 13.3 |
| Ctx | | | Occipital | | |
| | | | Ctx | | |
| AD 4 Temporal | 27.9 | 34.9 | Control | 80.1 | 76.3 |
| Ctx | | | (Path) 1 | | |
| | | | Occipital | | |
| | | | Ctx | | |
| AD 5 Inf | 59.0 | 100.0 | Control | 17.3 | 20.0 |
| Temporal Ctx | | | (Path) 2 | | |
| | | | Occipital | | |
| | | | Ctx | | |
| AD 5 | 33.2 | 44.1 | Control | 8.4 | 8.7 |
| SupTemporal | | | (Path) 3 | | |
| Ctx | | | Occipital | | |
| | | | Ctx | | |
| AD 6 Inf | 100.0 | 73.2 | Control | 21.2 | 20.6 |
| Temporal Ctx | | | (Path) 4 | | |
| | | | Occipital | | |
| | | | Ctx | | |
| AD 6 Sup | 79.6 | 80.1 | Control 1 | 12.1 | 16.3 |
| Temporal Ctx | | | Parietal Ctx | | |
| Control 1 | 10.2 | 13.7 | Control 2 | 48.0 | 40.9 |
| Temporal Ctx | | | Parietal Ctx | | |
| Control 2 | 41.2 | 31.9 | Control 3 | 17.9 | 16.3 |
| Temporal Ctx | | | Parietal Ctx | | |
| Control 3 | 20.3 | 20.0 | Control | 74.7 | 64.2 |
| Temporal Ctx | | | (Path) 1 | | |
| | | | Parietal Ctx | | |
| Control 4 | 9.7 | 9.9 | Control | 28.9 | 59.9 |
| Temporal Ctx | | | (Path) 2 | | |
| | | | Parietal Ctx | | |
| Control (Path) | 59.9 | 68.3 | Control | 10.2 | 9.0 |
| 1 Temporal Ctx | | | (Path) 3 | | |
| | | | Parietal Ctx | | |
| Control (Path) | 40.3 | 41.2 | Control | 44.8 | 43.8 |
| 2 Temporal Ctx | | | (Path) 4 | | |
| | | | Parietal Ctx | | |

TABLE NE
General_screening_panel_v1.4

| | Rel. Exp. (%) | | Rel. Exp. (%) |
|---|---|---|---|
| | Ag3373, Run | | Ag3373, Run |
| Tissue Name | 217043119 | Tissue Name | 217043119 |
| Adipose | 12.0 | Renal ca. TK-10 | 20.3 |
| Melanoma* | 30.8 | Bladder | 23.2 |
| Hs688(A).T | | | |
| Melanoma* | 69.3 | Gastric ca. (liver met.) | 25.3 |
| Hs688(B).T | | NCI-N87 | |
| Melanoma* M14 | 15.0 | Gastric ca. KATO III | 30.8 |
| Melanoma* | 26.6 | Colon ca. SW-948 | 9.7 |
| LOXIMVI | | | |
| Melanoma* SK- | 21.5 | Colon ca. SW480 | 35.1 |
| MEL-5 | | | |
| Squamous cell | 33.0 | Colon ca.* (SW480 | 13.9 |
| carcinoma SCC-4 | | met) SW620 | |
| Testis Pool | 19.8 | Colon ca. HT29 | 8.5 |
| Prostate ca.* (bone | 100.0 | Colon ca. HCT-116 | 36.9 |
| met) PC-3 | | | |
| Prostate Pool | 9.2 | Colon ca. CaCo-2 | 42.9 |
| Placenta | 3.8 | Colon cancer tissue | 9.0 |
| Uterus Pool | 7.4 | Colon ca. SW1116 | 5.8 |
| Ovarian ca. | 28.5 | Colon ca. Colo-205 | 4.3 |
| OVCAR-3 | | | |
| Ovarian ca. SK- | 40.3 | Colon ca. SW-48 | 4.2 |
| OV-3 | | | |
| Ovarian ca. | 20.0 | Colon Pool | 20.7 |
| OVCAR-4 | | | |
| Ovarian ca. | 35.1 | Small Intestine Pool | 12.2 |
| OVCAR-5 | | | |
| Ovarian ca. | 10.9 | Stomach Pool | 9.9 |
| IGROV-1 | | | |
| Ovarian ca. | 9.2 | Bone Marrow Pool | 11.6 |
| OVCAR-8 | | | |
| Ovary | 9.7 | Fetal Heart | 20.7 |
| Breast ca. MCF-7 | 37.6 | Heart Pool | 10.6 |
| Breast ca. MDA- | 37.1 | Lymph Node Pool | 17.9 |
| MB-231 | | | |
| Breast ca. BT 549 | 62.4 | Fetal Skeletal Muscle | 12.3 |
| Breast ca. T47D | 61.1 | Skeletal Muscle Pool | 16.0 |
| Breast ca. MDA-N | 10.0 | Spleen Pool | 11.6 |
| Breast Pool | 17.3 | Thymus Pool | 12.2 |
| Trachea | 12.0 | CNS cancer | 29.1 |
| | | (glio/astro) U87-MG | |
| Lung | 6.7 | CNS cancer | 69.3 |
| | | (glio/astro) U-118-MG | |
| Fetal Lung | 34.2 | CNS cancer | 34.9 |
| | | (neuro; met) SK-N-AS | |
| Lung ca. NCI-N417 | 5.4 | CNS cancer (astro) SF- | 19.1 |
| | | 539 | |
| Lung ca. LX-1 | 17.2 | CNS cancer (astro) | 35.8 |
| | | SNB-75 | |
| Lung ca. NCI-H146 | 3.0 | CNS cancer (glio) | 11.3 |
| | | SNB-19 | |
| Lung ca. SHP-77 | 18.6 | CNS cancer (glio) SF- | 26.4 |
| | | 295 | |
| Lung ca. A549 | 29.1 | Brain (Amygdala) Pool | 4.5 |
| Lung ca. NCI-H526 | 4.6 | Brain (cerebellum) | 8.1 |
| Lung ca. NCI-H23 | 31.6 | Brain (fetal) | 13.2 |
| Lung ca. NCI-H460 | 18.2 | Brain (Hippocampus) | 5.3 |
| | | Pool | |
| Lung ca. HOP-62 | 14.1 | Cerebral Cortex Pool | 5.4 |
| Lung ca. NCI-H522 | 31.6 | Brain (Substantia | 4.8 |
| | | nigra) Pool | |
| Liver | 1.2 | Brain (Thalamus) Pool | 8.0 |
| Fetal Liver | 32.3 | Brain (whole) | 6.2 |
| Liver ca. HepG2 | 14.6 | Spinal Cord Pool | 6.6 |
| Kidney Pool | 22.1 | Adrenal Gland | 8.1 |
| Fetal Kidney | 26.1 | Pituitary gland Pool | 3.0 |
| Renal ca. 786-0 | 28.7 | Salivary Gland | 4.7 |
| Renal ca. A498 | 11.3 | Thyroid (female) | 4.4 |
| Renal ca. ACHN | 12.2 | Pancreatic ca. | 17.3 |
| | | CAPAN2 | |
| Renal ca. UO-31 | 24.1 | Pancreas Pool | 17.1 |

TABLE NF
Panel 1.3D

| | Rel. Exp. (%) | | Rel. Exp. (%) |
|---|---|---|---|
| | Ag3023, Run | | Ag3023, Run |
| Tissue Name | 167966931 | Tissue Name | 167966931 |
| Liver adenocarcinoma | 51.1 | Kidney (fetal) | 26.2 |
| Pancreas | 6.1 | Renal ca. 786-0 | 34.2 |
| Pancreatic ca. CAPAN 2 | 17.7 | Renal ca. A498 | 17.6 |
| Adrenal gland | 3.8 | Renal ca. RXF 393 | 17.2 |
| Thyroid | 3.0 | Renal ca. ACHN | 13.5 |
| Salivary gland | 3.9 | Renal ca. UO-31 | 0.0 |
| Pituitary gland | 3.6 | Renal ca. TK-10 | 23.0 |
| Brain (fetal) | 8.1 | Liver | 11.7 |
| Brain (whole) | 8.5 | Liver (fetal) | 8.0 |
| Brain (amygdala) | 6.7 | Liver ca. | 26.2 |
| | | (hepatoblast) HepG2 | |
| Brain (cerebellum) | 15.2 | Lung | 3.1 |
| Brain (hippocampus) | 5.4 | Lung (fetal) | 11.0 |
| Brain (substantia nigra) | 9.0 | Lung ca. (small cell) | 12.9 |
| | | LX-1 | |
| Brain (thalamus) | 4.2 | Lung ca. (small cell) | 9.9 |
| | | NCI-H69 | |
| Cerebral Cortex | 2.0 | Lung ca. (s.cell var.) | 67.8 |
| | | SHP-77 | |
| Spinal cord | 6.9 | Lung ca. (large | 3.4 |
| | | cell)NCI-H460 | |
| Glio/astro U87-MG | 28.5 | Lung ca. (non-sm. | 45.1 |
| | | cell) A549 | |
| Glio/astro U-118-MG | 46.7 | Lung ca. (non-s.cell) | 22.7 |
| | | NCI-H23 | |
| astrocytoma SW1783 | 40.6 | Lung ca. (non-s.cell) | 25.7 |
| | | HOP-62 | |
| neuro*; met SK-N-AS | 27.2 | Lung ca. (non-s.cl) | 38.2 |
| | | NCI-H522 | |
| astrocytoma SF-539 | 29.7 | Lung ca. (squam.) | 27.4 |
| | | SW 900 | |
| astrocytoma SNB-75 | 35.1 | Lung ca. (squam.) | 29.9 |
| | | NCI-H596 | |
| glioma SNB-19 | 15.6 | Mammary gland | 5.1 |
| glioma U251 | 37.9 | Breast ca.* (pl.ef) | 47.0 |
| | | MCF-7 | |
| glioma SF-295 | 18.4 | Breast ca.* (pl.ef) | 22.7 |
| | | MDA-MB-231 | |
| Heart (fetal) | 2.9 | Breast ca.* (pl.ef) | 86.5 |
| | | T47D | |
| Heart | 12.9 | Breast ca. BT-549 | 15.9 |
| Skeletal muscle (fetal) | 3.4 | Breast ca. MDA-N | 10.4 |
| Skeletal muscle | 36.3 | Ovary | 2.9 |
| Bone marrow | 4.5 | Ovarian ca. | 26.1 |
| | | OVCAR-3 | |
| Thymus | 14.3 | Ovarian ca. | 16.3 |
| | | OVCAR-4 | |
| Spleen | 8.7 | Ovarian ca. | 83.5 |
| | | OVCAR-5 | |
| Lymph node | 11.8 | Ovarian ca. | 9.3 |
| | | OVCAR-8 | |
| Colorectal | 10.4 | Ovarian ca. IGROV-1 | 12.0 |
| Stomach | 7.8 | Ovarian ca.* | 100.0 |
| | | (ascites) SK-OV-3 | |
| Small intestine | 5.1 | Uterus | 4.9 |
| Colon ca. SW480 | 19.3 | Placenta | 1.3 |
| Colon ca.* | 42.9 | Prostate | 3.9 |
| SW620(SW480 met) | | | |
| Colon ca. HT29 | 9.9 | Prostate ca.* (bone | 78.5 |
| | | met)PC-3 | |
| Colon ca. HCT-116 | 26.2 | Testis | 9.7 |
| Colon ca. CaCo-2 | 41.5 | Melanoma | 5.9 |
| | | Hs688(A).T | |
| Colon ca. | 6.3 | Melanoma* (met) | 14.2 |
| tissue(ODO3866) | | Hs688(B).T | |
| Colon ca. HCC-2998 | 16.0 | Melanoma UACC- | 14.0 |
| | | 62 | |
| Gastric ca.* (liver met) | 18.8 | Melanoma M14 | 5.7 |
| NCI-N87 | | | |
| Bladder | 30.6 | Melanoma LOX | 8.8 |
| | | IMVI | |
| Trachea | 3.2 | Melanoma* (met) | 14.7 |
| | | SK-MEL-5 | |
| Kidney | 9.6 | Adipose | 18.9 |

TABLE NG
Panel 4D

| | Rel. | Rel. | | Rel. | Rel. |
|---|---|---|---|---|---|
| | Exp. (%) | Exp. (%) | | Exp. (%) | Exp. (%) |
| | Ag3023, | Ag3373, | | Ag3023, | Ag3373, |
| | Run | Run | | Run | Run |
| Tissue Name | 164516146 | 165296617 | Tissue Name | 164516146 | 165296617 |
| Secondary Th1 act | 18.6 | 17.9 | HUVEC IL-1beta | 20.3 | 18.6 |
| Secondary Th2 act | 24.3 | 28.5 | HUVEC IFN | 25.3 | 22.7 |
| | | | gamma | | |
| Secondary Tr1 act | 22.8 | 21.8 | HUVEC TNF | 16.3 | 18.0 |
| | | | alpha + IFN | | |
| | | | gamma | | |
| Secondary Th1 rest | 7.5 | 6.8 | HUVEC TNF | 18.2 | 13.4 |
| | | | alpha + IL4 | | |
| Secondary Th2 rest | 11.6 | 9.5 | HUVEC IL-11 | 13.7 | 9.9 |
| Secondary Tr1 rest | 12.1 | 10.7 | Lung | 25.7 | 21.6 |
| | | | Microvascular EC | | |
| | | | none | | |
| Primary Th1 act | 20.7 | 16.5 | Lung | 26.2 | 18.3 |
| | | | Microvascular EC | | |
| | | | TNF alpha + IL- | | |
| | | | 1beta | | |
| Primary Th2 act | 20.2 | 19.3 | Microvascular | 27.5 | 21.3 |
| | | | Dermal EC none | | |
| Primary Tr1 act | 23.3 | 27.7 | Microsvasular | 20.7 | 19.9 |
| | | | Dermal EC | | |
| | | | TNF alpha + IL- | | |
| | | | 1beta | | |
| Primary Th1 rest | 51.1 | 51.4 | Bronchial | 13.0 | 16.3 |
| | | | epithelium | | |
| | | | TNF alpha + | | |
| | | | IL1beta | | |
| Primary Th2 rest | 26.2 | 29.5 | Small airway | 8.1 | 8.5 |
| | | | epithelium none | | |
| Primary Tr1 rest | 23.7 | 26.1 | Small airway | 50.3 | 39.8 |
| | | | epithelium | | |
| | | | TNF alpha + IL- | | |
| | | | 1beta | | |
| CD45RA CD4 | 14.6 | 11.0 | Coronery artery | 20.2 | 18.9 |
| lymphocyte act | | | SMC rest | | |
| CD45RO CD4 | 25.2 | 22.4 | Coronery artery | 12.0 | 9.8 |
| lymphocyte act | | | SMC TNF alpha + | | |
| | | | IL-1beta | | |
| CD8 lymphocyte | 20.4 | 15.8 | Astrocytes rest | 10.4 | 11.1 |
| act | | | | | |
| Secondary CD8 | 16.5 | 19.9 | Astrocytes | 11.7 | 9.8 |
| lymphocyte rest | | | TNF alpha + IL- | | |
| | | | 1beta | | |
| Secondary CD8 | 13.2 | 9.3 | KU-812 | 47.6 | 38.2 |
| lymphocyte act | | | (Basophil) rest | | |
| CD4 lymphocyte | 17.1 | 11.6 | KU-812 | 94.0 | 92.0 |
| none | | | (Basophil) | | |
| | | | PMA/ionomycin | | |
| 2ry | 18.3 | 16.6 | CCD1106 | 19.9 | 13.2 |
| Th1/Th2/Tr1_anti- | | | (Keratinocytes) | | |
| CD95 CH11 | | | none | | |
| LAK cells rest | 25.5 | 16.0 | CCD1106 | 6.0 | 4.8 |
| | | | (Keratinocytes) | | |
| | | | TNF alpha + IL- | | |
| | | | 1beta | | |
| LAK cells IL-2 | 27.2 | 22.5 | Liver cirrhosis | 3.1 | 2.7 |
| LAK cells IL- | 27.2 | 19.3 | Lupus kidney | 2.1 | 1.7 |
| 2 + IL-12 | | | | | |
| LAK cells IL- | 36.3 | 34.4 | NCI-H292 none | 30.1 | 18.9 |
| 2 + IFN gamma | | | | | |
| LAK cells IL-2 + | 35.1 | 29.7 | NCI-H292 IL-4 | 33.9 | 34.6 |
| IL-18 | | | | | |
| LAK cells | 12.4 | 11.0 | NCI-H292 IL-9 | 40.1 | 29.1 |
| PMA/ionomycin | | | | | |
| NK Cells IL-2 rest | 20.0 | 15.0 | NCI-H292 IL-13 | 16.2 | 14.2 |
| Two Way MLR 3 | 24.0 | 16.7 | NCI-H292 IFN | 16.6 | 18.4 |
| day | | | gamma | | |
| Two Way MLR 5 | 12.9 | 10.1 | HPAEC none | 13.6 | 13.5 |
| day | | | | | |
| Two Way MLR 7 | 11.4 | 9.5 | HPAEC TNF | 25.3 | 25.3 |
| day | | | alpha + IL-1beta | | |
| PBMC rest | 13.7 | 10.5 | Lung fibroblast | 11.4 | 14.2 |
| | | | none | | |
| PBMC PWM | 69.3 | 66.4 | Lung fibroblast | 6.1 | 7.2 |
| | | | TNF alpha + IL- | | |
| | | | 1beta | | |
| PBMC PHA-L | 22.8 | 17.7 | Lung fibroblast | 28.5 | 29.1 |
| | | | IL-4 | | |
| Ramos (B cell) | 24.1 | 19.3 | Lung fibroblast | 23.0 | 23.3 |
| none | | | IL-9 | | |
| Ramos (B cell) | 100.0 | 100.0 | Lung fibroblast | 20.6 | 18.9 |
| ionomycin | | | IL-13 | | |
| B lymphocytes | 71.7 | 74.2 | Lung fibroblast | 39.0 | 32.5 |
| PWM | | | IFN gamma | | |
| B lymphocytes | 29.1 | 28.7 | Dermal fibroblast | 33.9 | 31.0 |
| CD40L and IL-4 | | | CCD1070 rest | | |
| EOL-1 dbcAMP | 12.1 | 10.5 | Dermal fibroblast | 76.8 | 62.0 |
| | | | CCD1070 TNF | | |
| | | | alpha | | |
| EOL-1 dbcAMP | 14.5 | 10.9 | Dermal fibroblast | 20.3 | 13.9 |
| PMA/ionomycin | | | CCD1070 IL- | | |
| | | | 1beta | | |
| Dendritic cells | 13.2 | 14.8 | Dermal fibroblast | 14.2 | 9.5 |
| none | | | IFN gamma | | |
| Dendritic cells LPS | 11.7 | 8.3 | Dermal fibroblast | 26.4 | 20.4 |
| | | | IL-4 | | |
| Dendritic cells | 17.7 | 12.7 | IBD Colitis 2 | 2.6 | 2.2 |
| anti-CD40 | | | | | |
| Monocytes rest | 16.7 | 17.6 | IBD Crohn's | 2.0 | 1.9 |
| Monocytes LPS | 6.4 | 5.0 | Colon | 11.9 | 10.5 |
| Macrophages rest | 23.5 | 22.8 | Lung | 13.3 | 11.2 |
| Macrophages LPS | 9.9 | 7.1 | Thymus | 14.4 | 12.9 |
| HUVEC none | 20.6 | 17.9 | Kidney | 27.5 | 19.6 |
| HUVEC starved | 43.5 | 38.4 | | | |

CNS_neurodegeneration_v1.0 Summary: Ag3023/Ag3373 This panel does not show differential expression of the CG56804-04 gene in Alzheimer's disease. However, this expression profile confirms the presence of this gene in the brain. Please see Panel 1.3D for discussion of utility of this gene in the central nervous system. Ag5847—This primer pair recognizes a splice variant of CG58564-01 designated CG58564-04. Expression of this variant is low/undetectable (CTs>35) across all of the samples on this panel (data not shown).

General_screening_panel_v1.4 Summary: Ag3373 Highest expression of the CG56804-04 gene is seen in a prostate cancer cell line (CT=27). Overall, this gene is expressed at moderate levels in the cancer cell lines in this panel. A higher level of expression is observed in clusters of cell lines derived from prostate, brain, melanoma, colon, lung, breast and ovarian cancer when compared to expression in normal prostate, brain, colon, lung, breast and ovary. Thus, this gene could potentially be used as a diagnostic marker of cancer in these tissues. Furthermore, inhibition of the activity of this gene product using small molecule drugs may be effective in the treatment of cancer in these tissues.

Among tissues with metabolic function, this gene product has moderate levels of expression in adipose, heart, skeletal muscle, adrenal, pituitary, thyroid and pancreas. Thus, this gene product may be a small molecule target for the treatment of endocrine and metabolic diseases, including obesity and Types 1 and 2 diabetes.

In addition, this gene appears to be differentially expressed in fetal (CT value=29) vs adult liver (CT value=33) and may be useful for differentiation between the two sources of this tissue.

This gene is also expressed at moderate levels in all central nervous system samples present on this panel. Please see Panel 1.3D for discussion of utility of this gene in the central nervous system.

General_screening_panel_v1.5 Summary: Ag5844—This primer pair recognizes a splice variant of CG58564-01. Expression of this variant is low/undetectable (CTs>35) across all of the samples on this panel (data not shown).

Panel 1.3D Summary: Ag3023 The CG56804-04 gene is ubiquitously expressed among the samples on this panel, with highest expression in an ovarian cancer cell line (CT=28.8). Overall, the expression of this gene shows good agreement with panel 1.4. A higher level of expression is observed in prostate, brain, melanoma, colon, lung, pancreatic, breast and ovarian cancer cell lines than the normal prostate, brain, colon, lung, pancreas, breast and ovary. Thus, expression of this gene could be used as a diagnostic marker of cancer in these tissues. Furthermore, inhibition of the activity of this gene product using small molecule drugs may be effective in the treatment of cancer in these tissues.

Among tissues with metabolic function, expression of this gene is widespread, as in the previous panel. Please see Panel 1.4 for discussion of utility of this gene in metabolic disease.

This gene represents a dual specificity phosphatase that is also expressed at low to moderate levels across the CNS. Dual-specificity phosphatases comprise a family of MAP kinase regulating enzymes, members of which are upregulated in brains subjected to insults such as ischemia and seizure activity. MAP kinases are known to regulate neurotrophic and neurotoxic pathways. Consequently, agents that modulate the activity of this gene may have utility in attenuating the apoptotic and neurodegenerative processes following brain insults.

Panel 41.1D Summary: Ag5844—This primer pair recognizes a splice variant of CG58564-01. Expression of this variant is low/undetectable (CTs>35) across all of the samples on this panel (data not shown).

Panel 4D Summary: Ag3023/Ag3373 The CG56804-04 gene is expressed at high to moderate levels in a wide range of cell types and tissues of significance in the immune response in health and disease. Highest expression of this gene is seen in ionomycin treated Ramos cells (CT=26.83). Therefore, targeting of ghis gene product with a small molecule drug or antibody therapeutic may modulate the functions of cells of the immune system as well as resident tissue cells and lead to improvement of the symptoms of patients suffering from autoimmune and inflammatory diseases such as asthma, allergies, inflammatory bowel disease, lupus erythematosus, and arthritis, including osteoarthritis and rheumatoid arthritis.

O. CG57819-01: RPGR-Interacting Protein-1

Expression of gene CG57819-01 was assessed using the primer-probe set Ag3338, described in Table OA. Results of the RTQ-PCR runs are shown in Tables OB and OC.

Table OA. Probe Name Ag3338

TABLE OA
Probe Name Ag3338

| | | | Start | SEQ ID |
|---|---|---|---|---|
| Primers | Sequences | Length | Position | NO: |
| Forward | 5′-cccattcagcactgaaacag-3′ | 20 | 3021 | 426 |
| Probe | TET-5′-tcctgtaaatgacaaagaatcctctgaaca-3′-TAMRA | 30 | 3055 | 427 |
| Reverse | 5′-tgcttcactgacttcagaacct-3′ | 22 | 3085 | 428 |

TABLE OB
General_screening_panel_v1.4

| | Rel. Exp. (%) | | Rel. Exp. (%) |
|---|---|---|---|
| | Ag3338, Run | | Ag3338, Run |
| Tissue Name | 215773746 | Tissue Name | 215773746 |
| Adipose | 1.1 | Renal ca. TK-10 | 0.8 |
| Melanoma* | 0.0 | Bladder | 1.1 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.2 | Colon ca. SW480 | 0.4 |
| MEL-5 | | | |
| Squamous cell | 0.0 | Colon ca.* (SW480 | 0.0 |
| carcinoma SCC-4 | | met) SW620 | |
| Testis Pool | 100.0 | Colon ca. HT29 | 0.5 |
| Prostate ca.* (bone | 0.0 | Colon ca. HCT-116 | 0.2 |
| met) PC-3 | | | |
| Prostate Pool | 1.0 | Colon ca. CaCo-2 | 1.0 |
| Placenta | 0.0 | Colon cancer tissue | 0.9 |
| Uterus Pool | 0.0 | Colon ca. SW1116 | 0.2 |
| Ovarian ca. | 0.9 | Colon ca. Colo-205 | 0.2 |
| OVCAR-3 | | | |
| Ovarian ca. SK- | 0.0 | Colon ca. SW-48 | 0.0 |
| OV-3 | | | |
| Ovarian ca. | 1.2 | Colon Pool | 0.5 |
| OVCAR-4 | | | |
| Ovarian ca. | 3.5 | Small Intestine Pool | 0.3 |
| OVCAR-5 | | | |
| Ovarian ca. | 0.0 | Stomach Pool | 0.2 |
| IGROV-1 | | | |
| Ovarian ca. | 0.0 | Bone Marrow Pool | 0.0 |
| OVCAR-8 | | | |
| Ovary | 0.9 | Fetal Heart | 0.8 |
| Breast ca. MCF-7 | 1.9 | Heart Pool | 1.1 |
| Breast ca. MDA- | 1.2 | Lymph Node Pool | 1.4 |
| MB-231 | | | |
| Breast ca. BT 549 | 0.2 | Fetal Skeletal Muscle | 0.2 |
| Breast ca. T47D | 6.7 | Skeletal Muscle Pool | 0.0 |
| Breast ca. MDA-N | 0.0 | Spleen Pool | 1.4 |
| Breast Pool | 0.5 | Thymus Pool | 0.0 |
| Trachea | 0.9 | CNS cancer | 0.0 |
| | | (glio/astro) U87-MG | |
| Lung | 0.2 | CNS cancer | 0.0 |
| | | (glio/astro) U-118-MG | |
| Fetal Lung | 0.4 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.8 | CNS cancer (astro) | 0.0 |
| | | SNB-75 | |
| Lung ca. NCI-H146 | 0.5 | CNS cancer (glio) | 0.0 |
| | | SNB-19 | |
| Lung ca. SHP-77 | 0.1 | CNS cancer (glio) SF- | 0.2 |
| | | 295 | |
| Lung ca. A549 | 1.5 | Brain (Amygdala) Pool | 0.7 |
| Lung ca. NCI-H526 | 0.0 | Brain (cerebellum) | 0.6 |
| Lung ca. NCI-H23 | 1.5 | Brain (fetal) | 0.9 |
| Lung ca. NCI-H460 | 0.0 | Brain (Hippocampus) | 0.7 |
| | | Pool | |
| Lung ca. HOP-62 | 3.0 | Cerebral Cortex Pool | 0.2 |
| Lung ca. NCI-H522 | 0.0 | Brain (Substantia | 0.7 |
| | | nigra) Pool | |
| Liver | 0.4 | Brain (Thalamus) Pool | 1.3 |
| Fetal Liver | 0.5 | Brain (whole) | 0.0 |
| Liver ca. HepG2 | 0.2 | Spinal Cord Pool | 0.9 |
| Kidney Pool | 0.9 | Adrenal Gland | 0.0 |
| Fetal Kidney | 0.6 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 3.4 |
| | | CAPAN2 | |
| Renal ca. UO-31 | 0.0 | Pancreas Pool | 0.8 |

TABLE OC
Panel 4D

| | Rel. Exp. (%) | | Rel. Exp. (%) |
|---|---|---|---|
| | Ag3338, Run | | Ag3338, Run |
| Tissue Name | 165221737 | Tissue Name | 165221737 |
| Secondary Th1 act | 0.0 | HUVEC IL-1beta | 0.0 |
| Secondary Th2 act | 0.0 | HUVEC IFN gamma | 6.9 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 2.6 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 1.9 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 4.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 4.6 | CCD1106 | 0.0 |
| | | (Keratinocytes) | |
| | | TNF alpha + IL-1beta | |
| LAK cells IL-2 | 0.0 | Liver cirrhosis | 0.0 |
| LAK cells IL-2 + IL-12 | 0.0 | Lupus kidney | 2.4 |
| LAK cells IL-2 + IFN | 0.0 | NCI-H292 none | 0.0 |
| gamma | | | |
| LAK cells IL-2 + IL-18 | 0.0 | NCI-H292 IL-4 | 4.5 |
| LAK cells | 3.1 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 14.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 3.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 4.7 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 13.9 | Dermal fibroblast IL-4 | 0.0 |
| Dendritic cells anti- | 6.0 | IBD Colitis 2 | 0.0 |
| CD40 | | | |
| Monocytes rest | 100.0 | IBD Crohn's | 0.0 |
| Monocytes LPS | 0.0 | Colon | 15.2 |
| Macrophages rest | 1.3 | Lung | 4.0 |
| Macrophages LPS | 0.0 | Thymus | 0.0 |
| HUVEC none | 0.0 | Kidney | 3.1 |
| HUVEC starved | 0.0 | | |

CNS_neurodegeneration_v1.0 Summary: Ag3338—Expression of this gene is low/undetectable (CTs>35) across all of the samples on this panel (data not shown).

General_screening_panel_v1.4 Summary: Ag3338—Expression of this gene is highest in testis (CT=29.4). Therefore, expression of this gene could be used to distinguish this sample from others on the panel.

There is also low expression in pancreatic cancer cell line CAPAN2, lung cancer cell line HOP-62, breast cancer cell line T47D, and ovarian cancer cell line OVCAR-5. Thus, expression of this gene could be used to differentiate these samples from other samples on this panel.

Panel 4D Summary: Ag3338—Significant expression of this gene is seen only in resting monocytes (CT=32.3) Therefore, expression of this gene can be used to differentiate between this sample and others on this panel.

P. CG57789-01 and CG57789-02: RAS-Like Protein RRP22-like

Expression of gene CG57789-01 and variant CG57789-02 was assessed using the primer-probe set Ag3333, described in Table PA. Results of the RTQ-PCR runs are shown in Tables PB, PC and PD.

Table PA. Probe Name Ag3333

TABLE PA
Probe Name Ag3333

| | | | Start | SEQ ID |
|---|---|---|---|---|
| Primers | Sequences | Length | Position | NO: |
| Forward | 5′-tcgactttccacccatcag-3′ | 19 | 181 | 429 |
| Probe | TET-5′-cttccctgtcaatacgctccaggagt-3′-TAMRA | 26 | 203 | 430 |
| Reverse | 5′-aggatgtaggcgtggacact-3′ | 20 | 258 | 431 |

TABLE PB
CNS_neurodegeneration_v1.0

| | Rel. Exp. (%) Ag3333, | | Rel. Exp. (%) Ag3333, |
|---|---|---|---|
| Tissue Name | Run 210146459 | Tissue Name | Run 210146459 |
| AD 1 Hippo | 22.2 | Control (Path) 3 | 7.5 |
| | | Temporal Ctx | |
| AD 2 Hippo | 18.8 | Control (Path) 4 | 21.6 |
| | | Temporal Ctx | |
| AD 3 Hippo | 17.9 | AD 1 Occipital Ctx | 29.7 |
| AD 4 Hippo | 8.7 | AD 2 Occipital Ctx | 0.0 |
| | | (Missing) | |
| AD 5 Hippo | 100.0 | AD 3 Occipital Ctx | 15.8 |
| AD 6 Hippo | 42.9 | AD 4 Occipital Ctx | 24.7 |
| Control 2 Hippo | 25.9 | AD 5 Occipital Ctx | 90.1 |
| Control 4 Hippo | 12.1 | AD 6 Occipital Ctx | 16.3 |
| Control (Path) 3 | 13.4 | Control 1 Occipital | 4.2 |
| Hippo | | Ctx | |
| AD 1 Temporal | 21.3 | Control 2 Occipital | 74.7 |
| Ctx | | Ctx | |
| AD 2 Temporal | 29.1 | Control 3 Occipital | 14.5 |
| Ctx | | Ctx | |
| AD 3 Temporal | 13.3 | Control 4 Occipital | 4.5 |
| Ctx | | Ctx | |
| AD 4 Temporal | 15.8 | Control (Path) 1 | 47.3 |
| Ctx | | Occipital Ctx | |
| AD 5 Inf Temporal | 92.0 | Control (Path) 2 | 13.5 |
| Ctx | | Occipital Ctx | |
| AD 5 Sup | 43.2 | Control (Path) 3 | 4.1 |
| Temporal Ctx | | Occipital Ctx | |
| AD 6 Inf Temporal | 26.4 | Control (Path) 4 | 14.6 |
| Ctx | | Occipital Ctx | |
| AD 6 Sup | 31.6 | Control 1 Parietal | 7.6 |
| Temporal Ctx | | Ctx | |
| Control 1 | 5.8 | Control 2 Parietal | 39.2 |
| Temporal Ctx | | Ctx | |
| Control 2 | 51.8 | Control 3 Parietal | 21.9 |
| Temporal Ctx | | Ctx | |
| Control 3 | 14.5 | Control (Path) 1 | 56.3 |
| Temporal Ctx | | Parietal Ctx | |
| Control 3 | 8.1 | Control (Path) 2 | 20.2 |
| Temporal Ctx | | Parietal Ctx | |
| Control (Path) 1 | 39.2 | Control (Path) 3 | 6.2 |
| Temporal Ctx | | Parietal Ctx | |
| Control (Path) 2 | 40.9 | Control (Path) 4 | 24.5 |
| Temporal Ctx | | Parietal Ctx | |

TABLE PC
General_screening_panel_v1.4

| | Rel. Exp. (%) | | Rel. Exp. (%) |
|---|---|---|---|
| | Ag3333, Run | | Ag3333, Run |
| Tissue Name | 216516940 | Tissue Name | 216516940 |
| Adipose | 4.4 | Renal ca. TK-10 | 40.1 |
| Melanoma* | 0.9 | Bladder | 5.0 |
| Hs688(A).T | | | |
| Melanoma* | 1.8 | Gastric ca. (liver met.) | 4.5 |
| Hs688(B).T | | NCI-N87 | |
| Melanoma* M14 | 2.7 | Gastric ca. KATO III | 20.0 |
| Melanoma* | 0.3 | Colon ca. SW-948 | 0.0 |
| LOXIMVI | | | |
| Melanoma* SK- | 0.9 | Colon ca. SW480 | 100.0 |
| MEL-5 | | | |
| Squamous cell | 0.1 | Colon ca.* (SW480 | 33.0 |
| carcinoma SCC-4 | | met) SW620 | |
| Testis Pool | 2.1 | Colon ca. HT29 | 5.0 |
| Prostate ca.* (bone | 2.4 | Colon ca. HCT-116 | 0.1 |
| met) PC-3 | | | |
| Prostate Pool | 0.5 | Colon ca. CaCo-2 | 37.1 |
| Placenta | 5.7 | Colon cancer tissue | 2.3 |
| Uterus Pool | 0.3 | Colon ca. SW1116 | 16.2 |
| Ovarian ca. | 52.9 | Colon ca. Colo-205 | 0.2 |
| OVCAR-3 | | | |
| Ovarian ca. SK- | 0.6 | Colon ca. SW-48 | 0.2 |
| OV-3 | | | |
| Ovarian ca. | 17.9 | Colon Pool | 2.2 |
| OVCAR-4 | | | |
| Ovarian ca. | 4.5 | Small Intestine Pool | 1.0 |
| OVCAR-5 | | | |
| Ovarian ca. | 0.9 | Stomach Pool | 0.9 |
| IGROV-1 | | | |
| Ovarian ca. | 15.4 | Bone Marrow Pool | 1.8 |
| OVCAR-8 | | | |
| Ovary | 4.2 | Fetal Heart | 10.9 |
| Breast ca. MCF-7 | 0.7 | Heart Pool | 2.8 |
| Breast ca. MDA- | 0.4 | Lymph Node Pool | 4.4 |
| MB-231 | | | |
| Breast ca. BT 549 | 42.0 | Fetal Skeletal Muscle | 1.1 |
| Breast ca. T47D | 13.0 | Skeletal Muscle Pool | 46.7 |
| Breast ca. MDA-N | 0.1 | Spleen Pool | 0.0 |
| Breast Pool | 2.4 | Thymus Pool | 2.3 |
| Trachea | 2.4 | CNS cancer | 0.9 |
| | | (glio/astro) U87-MG | |
| Lung | 0.2 | CNS cancer | 0.3 |
| | | (glio/astro) U-118-MG | |
| Fetal Lung | 0.9 | CNS cancer | 69.7 |
| | | (neuro; met) SK-N-AS | |
| Lung ca. NCI-N417 | 17.1 | CNS cancer (astro) SF- | 2.2 |
| | | 539 | |
| Lung ca. LX-1 | 1.1 | CNS cancer (astro) | 15.9 |
| | | SNB-75 | |
| Lung ca. NCI-H146 | 14.5 | CNS cancer (glio) | 0.6 |
| | | SNB-19 | |
| Lung ca. SHP-77 | 37.6 | CNS cancer (glio) SF- | 6.0 |
| | | 295 | |
| Lung ca. A549 | 0.4 | Brain (Amygdala) Pool | 28.5 |
| Lung ca. NCI-H526 | 23.5 | Brain (cerebellum) | 29.1 |
| Lung ca. NCI-H23 | 8.2 | Brain (fetal) | 21.3 |
| Lung ca. NCI-H460 | 14.3 | Brain (Hippocampus) | 27.7 |
| | | Pool | |
| Lung ca. HOP-62 | 1.7 | Cerebral Cortex Pool | 36.1 |
| Lung ca. NCI-H522 | 86.5 | Brain (Substantia | 40.1 |
| | | nigra) Pool | |
| Liver | 1.6 | Brain (Thalamus) Pool | 37.6 |
| Fetal Liver | 0.7 | Brain (whole) | 59.5 |
| Liver ca. HepG2 | 6.2 | Spinal Cord Pool | 12.3 |
| Kidney Pool | 3.8 | Adrenal Gland | 4.7 |
| Fetal Kidney | 7.4 | Pituitary gland Pool | 3.7 |
| Renal ca. 786-0 | 0.2 | Salivary Gland | 48.0 |
| Renal ca. A498 | 20.9 | Thyroid (female) | 1.1 |
| Renal ca. ACHN | 8.5 | Pancreatic ca. | 0.0 |
| | | CAPAN2 | |
| Renal ca. UO-31 | 3.0 | Pancreas Pool | 4.0 |

TABLE PD
Panel 4D

| | Rel. Exp. (%) | | Rel. Exp. (%) |
|---|---|---|---|
| | Ag3333, Run | | Ag3333, Run |
| Tissue Name | 165084139 | Tissue Name | 165084139 |
| Secondary Th1 act | 0.8 | HUVEC IL-1beta | 0.0 |
| Secondary Th2 act | 3.0 | HUVEC IFN gamma | 0.5 |
| Secondary Tr1 act | 0.6 | HUVEC TNF alpha + | 0.8 |
| | | IFN gamma | |
| Secondary Th1 rest | 0.0 | HUVEC TNF alpha + | 0.0 |
| | | IL4 | |
| Secondary Th2 rest | 0.5 | HUVEC IL-11 | 0.3 |
| Secondary Tr1 rest | 0.0 | Lung Microvascular EC | 0.6 |
| | | none | |
| Primary Th1 act | 5.7 | Lung Microvascular EC | 0.4 |
| | | TNF alpha + IL-1beta | |
| Primary Th2 act | 9.8 | Microvascular Dermal | 0.0 |
| | | EC none | |
| Primary Tr1 act | 3.8 | Microsvasular Dermal | 0.4 |
| | | EC TNF alpha + IL-1beta | |
| Primary Th1 rest | 0.0 | Bronchial epithelium | 1.1 |
| | | TNF alpha + IL1beta | |
| Primary Th2 rest | 0.4 | Small airway epithelium | 1.9 |
| | | none | |
| Primary Tr1 rest | 0.6 | Small airway epithelium | 1.4 |
| | | TNF alpha + IL-1beta | |
| CD45RA CD4 | 4.1 | Coronery artery SMC rest | 1.7 |
| lymphocyte act | | | |
| CD45RO CD4 | 1.7 | Coronery artery SMC | 1.2 |
| lymphocyte act | | TNF alpha + IL-1beta | |
| CD8 lymphocyte act | 1.4 | Astrocytes rest | 100.0 |
| Secondary CD8 | 7.4 | Astrocytes TNF alpha + | 59.9 |
| lymphocyte rest | | IL-1beta | |
| Secondary CD8 | 0.0 | KU-812 (Basophil) rest | 2.0 |
| lymphocyte act | | | |
| CD4 lymphocyte none | 0.8 | KU-812 (Basophil) | 4.1 |
| | | PMA/ionomycin | |
| 2ry Th1/Th2/Tr1_anti- | 0.5 | CCD1106 | 12.5 |
| CD95 CH11 | | (Keratinocytes) none | |
| LAK cells rest | 0.5 | CCD1106 | 6.2 |
| | | (Keratinocytes) | |
| | | TNF alpha + IL-1beta | |
| LAK cells IL-2 | 0.3 | Liver cirrhosis | 0.9 |
| LAK cells IL-2 + IL-12 | 0.5 | Lupus kidney | 3.9 |
| LAK cells IL-2 + IFN | 0.0 | NCI-H292 none | 29.3 |
| gamma | | | |
| LAK cells IL-2 + IL-18 | 0.6 | NCI-H292 IL-4 | 39.5 |
| LAK cells | 0.3 | NCI-H292 IL-9 | 23.3 |
| PMA/ionomycin | | | |
| NK Cells IL-2 rest | 0.0 | NCI-H292 IL-13 | 21.9 |
| Two Way MLR 3 day | 0.8 | NCI-H292 IFN gamma | 14.5 |
| Two Way MLR 5 day | 0.9 | HPAEC none | 0.5 |
| Two Way MLR 7 day | 0.0 | HPAEC TNF alpha + IL- | 0.0 |
| | | 1beta | |
| PBMC rest | 0.0 | Lung fibroblast none | 4.5 |
| PBMC PWM | 8.1 | Lung fibroblast TNF | 2.2 |
| | | alpha + IL-1beta | |
| PBMC PHA-L | 11.6 | Lung fibroblast IL-4 | 12.9 |
| Ramos (B cell) none | 0.0 | Lung fibroblast IL-9 | 9.2 |
| Ramos (B cell) | 0.0 | Lung fibroblast IL-13 | 8.5 |
| ionomycin | | | |
| B lymphocytes PWM | 15.4 | Lung fibroblast IFN | 8.4 |
| | | gamma | |
| B lymphocytes CD40L | 2.1 | Dermal fibroblast | 40.6 |
| and IL-4 | | CCD1070 rest | |
| EOL-1 dbcAMP | 0.0 | Dermal fibroblast | 20.9 |
| | | CCD1070 TNF alpha | |
| EOL-1 dbcAMP | 0.0 | Dermal fibroblast | 19.3 |
| PMA/ionomycin | | CCD1070 IL-1beta | |
| Dendritic cells none | 0.0 | Dermal fibroblast IFN | 1.8 |
| | | gamma | |
| Dendritic cells LPS | 0.5 | Dermal fibroblast IL-4 | 3.8 |
| Dendritic cells anti- | 0.0 | IBD Colitis 2 | 0.0 |
| CD40 | | | |
| Monocytes rest | 0.0 | IBD Crohn's | 2.5 |
| Monocytes LPS | 0.0 | Colon | 4.2 |
| Macrophages rest | 0.0 | Lung | 9.1 |
| Macrophages LPS | 0.0 | Thymus | 11.3 |
| HUVEC none | 0.4 | Kidney | 2.6 |
| HUVEC starved | 0.6 | | |

CNS_neurodegeneration_v1.0 Summary: This panel confirms the expression of this gene in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3333 This gene is expressed at moderate to low levels in many of the samples on this panel, with the highest expression in colon cancer cell line SW480 (CT=27.8). Expression is significantly lower in SW680, a cell line derived from a metastasis of the primary tumor represented by SW480. Thus, expression of this gene could be used to differentiate between these two cell lines and potentially between primary colon cancer and its metastases.

Based on expression in this panel, this gene may be involved in gastric, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

This gene product is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes

This gene is expressed at low levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3333 The CG57789-01 gene is expressed at moderate to low levels in several samples on this panel, with the highest expression in resting astrocytes (CT=28.4). Moderate expression of this gene is seen in treated and untreated dermal and lung fibroblasts and the airway epithelial tumor line NCI-H292 cells. Thus, the transcript or the protein it encodes may be involved in pathological and inflammatory skin and lung conditions, including psoriasis, asthma, allergy, emphysema, and COPD.

Q. CG57758-01 and CG57758-02: Sodium/Lithium-Dependent Dicarboxylate Transporter

Expression of gene CG57758-01, a splice variant of CG57758-02, and CG57758-02 was assessed using the primer-probe sets Ag3326 and Ag3692, described in Tables QA and QB. Results of the RTQ-PCR runs are shown in Tables QC, QD, QE and QF.

Table QA. Probe Name Ag3326

TABLE QA
Probe Name Ag3326

| | | | Start | SEQ ID |
|---|---|---|---|---|
| Primers | Sequences | Length | Position | NO: |
| Forward | 5′-ccatttactggtgcacagaagt-3′ | 22 | 149 | 432 |
| Probe | TET-5′-atccctctggctgtcacctctctcat-3′-TAMRA | 26 | 172 | 433 |
| Reverse | 5′-ggagtccagaatctggaagagt-3′ | 22 | 216 | 434 |

Table QB. Probe Name Ag3692

TABLE QB
Probe Name Ag3692

| | | | Start | SEQ ID |
|---|---|---|---|---|
| Primers | Sequences | Length | Position | NO: |
| Forward | 5′-ccatttactggtgcacagaagt-3′ | 22 | 149 | 435 |
| Probe | TET-5′-atccctctggctgtcacctctctcat-3′-TAMRA | 26 | 172 | 436 |
| Reverse | 5′-ggagtccagaatctggaagagt-3′ | 22 | 216 | 437 |

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3326, | Ag3692, | Ag3692, | | Ag3326, | Ag3692, | Ag3692, |
| Tissue | Run | Run | Run | Tissue | Run | Run | Run |
| Name | 210144197 | 211145262 | 224337942 | Name | 210144197 | 211145262 | 224337942 |
| AD 1 | 2.1 | 4.3 | 1.0 | Control | 8.5 | 15.3 | 12.0 |
| Hippo | | | | (Path) 3 | | | |
| | | | | Temporal | | | |
| | | | | Ctx | | | |
| AD 2 | 20.9 | 28.3 | 25.0 | Control | 31.2 | 36.6 | 52.1 |
| Hippo | | | | (Path) 4 | | | |
| | | | | Temporal | | | |
| | | | | Ctx | | | |
| AD 3 | 0.0 | 0.9 | 0.6 | AD 1 | 2.7 | 3.0 | 0.0 |
| Hippo | | | | Occipital | | | |
| | | | | Ctx | | | |
| AD 4 | 2.1 | 7.1 | 2.6 | AD 2 | 0.0 | 0.0 | 0.0 |
| Hippo | | | | Occipital | | | |
| | | | | Ctx | | | |
| | | | | (Missing) | | | |
| AD 5 | 72.7 | 97.9 | 85.3 | AD 3 | 1.5 | 7.2 | 1.3 |
| hippo | | | | Occipital | | | |
| | | | | Ctx | | | |
| AD 6 | 13.7 | 18.3 | 5.5 | AD 4 | 71.7 | 35.6 | 30.6 |
| Hippo | | | | Occipital | | | |
| | | | | Ctx | | | |
| Control 2 | 14.5 | 20.2 | 15.2 | AD 5 | 25.3 | 31.9 | 12.4 |
| Hippo | | | | Occipital | | | |
| | | | | Ctx | | | |
| Control 4 | 11.7 | 7.4 | 5.1 | AD 6 | 17.2 | 19.1 | 11.2 |
| Hippo | | | | Occipital | | | |
| | | | | Ctx | | | |
| Control | 6.7 | 4.4 | 4.5 | Control 1 | 7.0 | 9.0 | 8.1 |
| (Path) 3 | | | | Occipital | | | |
| Hippo | | | | Ctx | | | |
| AD 1 | 4.0 | 1.7 | 2.8 | Control 2 | 33.2 | 44.8 | 26.1 |
| Temporal | | | | Occipital | | | |
| Ctx | | | | Ctx | | | |
| AD 2 | 80.7 | 50.7 | 37.4 | Control 3 | 30.1 | 37.6 | 21.9 |
| Temporal | | | | Occipital | | | |
| Ctx | | | | Ctx | | | |
| AD 3 | 3.6 | 0.0 | 1.1 | Control 4 | 16.3 | 12.6 | 8.2 |
| Temporal | | | | Occipital | | | |
| Ctx | | | | Ctx | | | |
| AD 4 | 19.5 | 30.6 | 15.2 | Control | 42.0 | 55.9 | 52.9 |
| Temporal | | | | (Path) 1 | | | |
| Ctx | | | | Occipital | | | |
| | | | | Ctx | | | |
| AD 5 Inf | 100.0 | 100.0 | 99.3 | Control | 6.7 | 13.0 | 7.7 |
| Temporal | | | | (Path) 2 | | | |
| Ctx | | | | Occipital | | | |
| | | | | Ctx | | | |
| AD 5 | 32.8 | 29.1 | 33.2 | Control | 8.7 | 6.6 | 5.4 |
| SupTemporal | | | | (Path) 3 | | | |
| Ctx | | | | Occipital | | | |
| | | | | Ctx | | | |
| AD 6 Inf | 27.7 | 21.3 | 26.6 | Control | 8.1 | 9.0 | 7.4 |
| Temporal | | | | (Path) 4 | | | |
| Ctx | | | | Occipital | | | |
| | | | | Ctx | | | |
| AD 6 Sup | 41.8 | 53.6 | 17.0 | Control 1 | 21.2 | 23.0 | 15.3 |
| Temporal | | | | Parietal | | | |
| Ctx | | | | Ctx | | | |
| Control 1 | 12.0 | 33.9 | 18.3 | Control 2 | 48.6 | 38.2 | 22.1 |
| Temporal | | | | Parietal | | | |
| Ctx | | | | Ctx | | | |
| Control 2 | 30.1 | 49.3 | 44.4 | Control 3 | 28.3 | 34.4 | 32.8 |
| Temporal | | | | Parietal | | | |
| Ctx | | | | Ctx | | | |
| Control 3 | 38.7 | 39.5 | 33.4 | Control | 78.5 | 97.3 | 100.0 |
| Temporal | | | | (Path) 1 | | | |
| Ctx | | | | Parietal | | | |
| | | | | Ctx | | | |
| Control 4 | 17.6 | 25.2 | 24.1 | Control | 50.7 | 50.7 | 37.9 |
| Temporal | | | | (Path) 2 | | | |
| Ctx | | | | Parietal | | | |
| | | | | Ctx | | | |
| Control | 69.7 | 70.7 | 49.7 | Control | 10.7 | 10.1 | 9.6 |
| (Path) 1 | | | | (Path) 3 | | | |
| Temporal | | | | Parietal | | | |
| Ctx | | | | Ctx | | | |
| Control | 35.4 | 50.7 | 33.4 | Control | 30.6 | 24.5 | 40.9 |
| (Path) 2 | | | | (Path) 4 | | | |
| Temporal | | | | Parietal | | | |
| Ctx | | | | Ctx | | | |

TABLE QD
General_screening_panel_v1.4

| | Rel. Exp. (%) | Rel. Exp. (%) | | Rel. Exp. (%) | Rel. Exp. (%) |
|---|---|---|---|---|---|
| | Ag3326, Run | Ag3692, Run | | Ag3326, Run | Ag3692, Run |
| Tissue Name | 215678613 | 217131191 | Tissue Name | 215678613 | 217131191 |
| Adipose | 0.0 | 0.0 | Renal ca. TK-10 | 11.4 | 12.0 |
| Melanoma* | 0.0 | 0.0 | Bladder | 0.0 | 0.1 |
| Hs688(A).T | | | | | |
| Melanoma* | 0.1 | 0.0 | Gastric ca. (liver | 0.0 | 0.0 |
| Hs688(B).T | | | met.) NCI-N87 | | |
| Melanoma* | 0.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. | 0.0 | 0.0 |
| SK-MEL-5 | | | SW480 | | |
| Squamous | 0.9 | 0.7 | Colon ca.* | 0.0 | 0.0 |
| cell | | | (SW480 met) | | |
| carcinoma | | | SW620 | | |
| SCC-4 | | | | | |
| Testis Pool | 0.1 | 0.2 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 0.0 | 0.0 | Colon ca. CaCo-2 | 0.0 | 0.0 |
| Placenta | 0.0 | 0.0 | Colon cancer | 0.1 | 0.0 |
| | | | tissue | | |
| Uterus Pool | 0.0 | 0.0 | Colon ca. | 0.0 | 0.0 |
| | | | SW1116 | | |
| Ovarian ca. | 0.0 | 0.0 | Colon ca. Colo- | 0.0 | 0.0 |
| OVCAR-3 | | | 205 | | |
| Ovarian ca. | 0.0 | 0.0 | Colon ca. SW-48 | 0.0 | 0.0 |
| SK-OV-3 | | | | | |
| Ovarian ca. | 0.1 | 0.0 | Colon Pool | 0.6 | 0.0 |
| OVCAR-4 | | | | | |
| Ovarian ca. | 0.0 | 0.0 | Small Intestine | 0.1 | 0.0 |
| OVCAR-5 | | | Pool | | |
| Ovarian ca. | 0.0 | 0.0 | Stomach Pool | 0.0 | 0.0 |
| IGROV-1 | | | | | |
| Ovarian ca. | 2.8 | 2.2 | Bone Marrow | 0.0 | 0.1 |
| OVCAR-8 | | | Pool | | |
| Ovary | 0.7 | 0.6 | Fetal Heart | 0.0 | 0.0 |
| Breast ca. | 0.0 | 0.0 | Heart Pool | 0.0 | 0.0 |
| MCF-7 | | | | | |
| Breast ca. | 0.0 | 0.0 | Lymph Node | 0.1 | 0.0 |
| MDA-MB- | | | Pool | | |
| 231 | | | | | |
| Breast ca. BT | 0.6 | 0.8 | Fetal Skeletal | 0.0 | 0.0 |
| 549 | | | Muscle | | |
| Breast ca. | 0.0 | 0.0 | Skeletal Muscle | 0.0 | 0.0 |
| T47D | | | Pool | | |
| Breast ca. | 0.0 | 0.0 | Spleen Pool | 0.4 | 0.2 |
| MDA-N | | | | | |
| Breast Pool | 0.0 | 0.1 | Thymus Pool | 0.0 | 0.0 |
| Trachea | 0.2 | 0.1 | CNS cancer | 0.0 | 0.0 |
| | | | (glio/astro) U87- | | |
| | | | MG | | |
| Lung | 0.0 | 0.0 | CNS cancer | 0.0 | 0.0 |
| | | | (glio/astro) U- | | |
| | | | 118-MG | | |
| Fetal Lung | 0.2 | 0.1 | CNS cancer | 0.0 | 0.0 |
| | | | (neuro; met) SK- | | |
| | | | N-AS | | |
| Lung ca. | 0.0 | 0.0 | CNS cancer | 0.0 | 0.0 |
| NCI-N417 | | | (astro) SF-539 | | |
| Lung ca. LX-1 | 0.0 | 0.0 | CNS cancer | 0.0 | 0.0 |
| | | | (astro) SNB-75 | | |
| Lung ca. | 0.0 | 0.0 | CNS cancer | 0.0 | 0.0 |
| NCI-H146 | | | (glio) SNB-19 | | |
| Lung ca. | 0.0 | 0.0 | CNS cancer | 0.1 | 0.1 |
| SHP-77 | | | (glio) SF-295 | | |
| Lung ca. | 0.0 | 0.1 | Brain | 0.4 | 0.4 |
| A549 | | | (Amygdala) Pool | | |
| Lung ca. | 2.0 | 0.0 | Brain | 1.4 | 1.0 |
| NCI-H526 | | | (cerebellum) | | |
| Lung ca. | 0.7 | 0.6 | Brain (fetal) | 0.7 | 0.4 |
| NCI-H23 | | | | | |
| Lung ca. | 0.0 | 0.0 | Brain | 0.5 | 0.7 |
| NCI-H460 | | | (Hippocampus) | | |
| | | | Pool | | |
| Lung ca. | 0.1 | 0.2 | Cerebral Cortex | 1.4 | 1.5 |
| HOP-62 | | | Pool | | |
| Lung ca. | 0.0 | 0.0 | Brain (Substantia | 1.4 | 1.4 |
| NCI-H522 | | | nigra) Pool | | |
| Liver | 28.7 | 24.1 | Brain | 1.1 | 0.9 |
| | | | (Thalamus) Pool | | |
| Fetal Liver | 100.0 | 100.0 | Brain (whole) | 4.1 | 3.7 |
| Liver ca. | 29.5 | 26.2 | Spinal Cord Pool | 0.1 | 0.2 |
| HepG2 | | | | | |
| Kidney Pool | 0.0 | 0.0 | Adrenal Gland | 2.6 | 1.9 |
| Fetal Kidney | 0.1 | 0.1 | Pituitary gland | 0.0 | 0.2 |
| | | | Pool | | |
| Renal ca. | 0.0 | 0.0 | Salivary Gland | 40.9 | 35.1 |
| 786-0 | | | | | |
| Renal ca. | 0.0 | 0.0 | Thyroid (female) | 0.0 | 0.0 |
| A498 | | | | | |
| Renal ca. | 0.0 | 0.0 | Pancreatic ca. | 0.5 | 0.8 |
| ACHN | | | CAPAN2 | | |
| Renal ca. | 0.0 | 0.0 | Pancreas Pool | 0.0 | 0.0 |
| UO-31 | | | | | |

TABLE QE
Panel 4.1D

| | Rel. Exp. (%) | | Rel. Exp. (%) |
|---|---|---|---|
| | Ag3692, Run | | Ag3692, Run |
| Tissue Name | 169987356 | Tissue Name | 169987356 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 11.3 |
| | | EC none | |
| Primary Tr1 act | 4.2 | Microsvasular Dermal | 0.0 |
| | | EC TNF alpha + IL-1beta | |
| Primary Th1 rest | 0.0 | Bronchial epithelium | 28.5 |
| | | TNF alpha + IL1beta | |
| Primary Th2 rest | 0.0 | Small airway epithelium | 5.7 |
| | | none | |
| Primary Tr1 rest | 0.0 | Small airway epithelium | 0.0 |
| | | TNF alpha + IL-1beta | |
| CD45RA CD4 | 3.9 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 3.6 |
| lymphocyte act | | | |
| CD4 lymphocyte none | 0.0 | KU-812 (Basophil) | 4.3 |
| | | PMA/ionomycin | |
| 2ry Th1/Th2/Tr1_anti- | 0.0 | CCD1106 | 10.7 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 94.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 3.2 | HPAEC TNF alpha + IL- | 0.0 |
| | | 1beta | |
| Two Way MLR 7 day | 0.0 | Lung fibroblast none | 0.0 |
| PBMC rest | 0.0 | Lung fibroblast TNF | 0.0 |
| | | alpha + IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 2.4 |
| HUVEC none | 0.0 | Kidney | 100.0 |
| HUVEC starved | 0.0 | | |

TABLE QF
Panel 5 Islet

| | Rel. Exp. (%) | | Rel. Exp. (%) |
|---|---|---|---|
| | Ag3326, Run | | Ag3326, Run |
| Tissue Name | 242385365 | Tissue Name | 242385365 |
| 97457_Patient- | 0.0 | 94709_Donor 2 AM - A_adipose | 0.2 |
| 02go_adipose | | | |
| 97476_Patient- | 0.0 | 94710_Donor 2 AM - B_adipose | 0.0 |
| 07sk_skeletal muscle | | | |
| 97477_Patient- | 0.0 | 94711_Donor 2 AM - C_adipose | 0.0 |
| 07ut_uterus | | | |
| 97478_Patient- | 0.0 | 94712_Donor 2 AD - A_adipose | 0.0 |
| 07pl_placenta | | | |
| 99167_Bayer Patient 1 | 0.3 | 94713_Donor 2 AD - B_adipose | 0.0 |
| 97482_Patient- | 0.0 | 94714_Donor 2 AD - C_adipose | 0.0 |
| 08ut_uterus | | | |
| 97483_Patient- | 0.0 | 94742_Donor 3 U - | 0.0 |
| 08pl_placenta | | A_Mesenchymal Stem Cells | |
| 97486_Patient- | 0.0 | 94743_Donor 3 U - | 0.0 |
| 09sk_skeletal muscle | | B_Mesenchymal Stem Cells | |
| 97487_Patient- | 0.0 | 94730_Donor 3 AM - A_adipose | 0.0 |
| 09ut_uterus | | | |
| 97488_Patient- | 0.0 | 94731_Donor 3 AM - B_adipose | 0.0 |
| 09pl_placenta | | | |
| 97492_Patient- | 0.0 | 94732_Donor 3 AM - C_adipose | 0.0 |
| 10ut_uterus | | | |
| 97493_Patient- | 0.0 | 94733_Donor 3 AD - A_adipose | 0.0 |
| 10pl_placenta | | | |
| 97495_Patient- | 0.0 | 94734_Donor 3 AD - B_adipose | 0.0 |
| 11go_adipose | | | |
| 97496_Patient- | 0.0 | 94735_Donor 3 AD - C_adipose | 0.0 |
| 11sk_skeletal muscle | | | |
| 97497_Patient- | 0.0 | 77138_Liver_HepG2untreated | 100.0 |
| 11ut_uterus | | | |
| 97498_Patient- | 0.0 | 73556_Heart_Cardiac stromal | 0.0 |
| 11pl_placenta | | cells (primary) | |
| 97500_Patient- | 0.1 | 81735_Small Intestine | 39.5 |
| 12go_adipose | | | |
| 97501_Patient- | 0.3 | 72409_Kidney_Proximal | 0.0 |
| 12sk_skeletal muscle | | Convoluted Tubule | |
| 97502_Patient- | 0.0 | 82685_Small | 0.0 |
| 12ut_uterus | | intestine_Duodenum | |
| 97503_Patient- | 0.0 | 90650_Adrenal_Adrenocortical | 0.0 |
| 12pl_placenta | | adenoma | |
| 94721_Donor 2 U - | 0.0 | 72410_Kidney_HRCE | 0.0 |
| A_Mesenchymal | | | |
| Stem Cells | | | |
| 94722_Donor 2 U - | 0.0 | 72411_Kidney_HRE | 0.0 |
| B_Mesenchymal | | | |
| Stem Cells | | | |
| 94723_Donor 2 U - | 0.0 | 73139_Uterus_Uterine smooth | 0.0 |
| C_Mesenchymal | | muscle cells | |
| Stem Cells | | | |

CNS_neurodegeneration_v1.0 Summary: Ag3326/Ag3692—Three experiments done with two primer pairs (same sequence) are in excellent agreement. This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3326/Ag3692 Two experiments with the smae probe and primer set produce results that are in excellent agreement. This gene is highly expressed in fetal liver (CT=26.5-27.0) and moderately expressed in adult liver (CT=28.5-28.8) and liver cancer cell line HepG2 (CT=28.4-28.8). This result agrees with the results seen in Panel 5 (expression in HepG2 (CT=29.2). These results are in agreement with published data that show a novel sodium dicarboxylate transporter in brain, choroid plexus kidney, intestine and liver. Thus, expression of this gene could be used to differentiate between these samples and other samples on this panel and as a marker for liver derived tissue.

This gene is expressed at low levels throughout the CNS, including in amygdala, substantia nigra, thalamus, cerebellum, and cerebral cortex. Therefore, this gene may play a role in central nervous system disorders such as Parkinson's disease, epilepsy, multiple sclerosis, schizophrenia and depression.

Low but significant levels of expression are also seen in the adrenal gland. Thus, this gene product may also be involved in metabolic disorders of this gland, including adrenoleukodystrophy and congenital adrenal hyperplasia.

### REFERENCES

1. Pajor A M, Gangula R, Yao X. Cloning and functional characterization of a high-affinity Na(+)/dicarboxylate cotransporter from mouse brain. Am J Physiol Cell Physiol May 2001;280(5):C1215-23.

2. Chen X Z, Shayakul C, Berger UV, Tian W, Hediger M A. Characterization of a rat Na+-dicarboxylate cotransporter. J Biol Chem Aug. 14, 1998;273(33):20972-81.

Panel 4.1D Summary: Ag3692 Significant expression of this gene is seen only in kidney and a liver cirrhosis sample (CTs=34.0). These results confirm that this gene is expressed in liver derived samples. The presence in the kidney is also in agreement with published results. Please see Panel 1.4. This gene product may be involved in maintaining or restoring normal function to the kidney during inflammation.

Panel 4D Summary: Ag3326 Results from one experiment are not included. The amp plot indicates that there were experimental difficulties with this run.

Panel 5 Islet Summary: Ag3326—The highest expression of this gene is in liver cancer cell line HepG2 (CT=29.2). There is also moderate expression in the small intestine (CT=30.5). These results compare well with previously published reports of sodium dicarboxylate transporter expression in mouse and rat (see discussion Panel 1.4).

R. CG57758-04 and CG57758-05: Sodium:Sulfate Symporter

Expression of gene CG57758-04 and CG57758-05, both splice variants of CG577584-01, was assessed using the primer-probe sets Ag3326, Ag3692 and Ag5818, described in Tables RA, RB and RC. Results of the RTQ-PCR runs are shown in Tables RD, RE, RF, RG and RH.

Table RA. Probe Name Ag3326

TABLE RA
Probe Name Ag3326

| | | | Start | SEQ ID |
|---|---|---|---|---|
| Primers | Sequences | Length | Position | NO: |
| Forward | 5′-ccatttactggtgcacagaagt-3′ | 22 | 138 | 438 |
| Probe | TET-5′-atccctctggctgtcacctctctcat-3′-TAMRA | 26 | 161 | 439 |
| Reverse | 5′-ggagtccagaatctggaagagt-3′ | 22 | 205 | 440 |

Table RB. Probe Name Ag3692

TABLE RB
Probe Name Ag3692

| | | | Start | SEQ ID |
|---|---|---|---|---|
| Primers | Sequences | Length | Position | NO: |
| Forward | 5′-ccatttactggtgcacagaagt-3′ | 22 | 138 | 441 |
| Probe | TET-5′-atccctctggctgtcacctctctcat-3′-TAMRA | 26 | 161 | 442 |
| Reverse | 5′-ggagtccagaatctggaagagt-3′ | 22 | 205 | 443 |

Table RC. Probe Name Ag5818

TABLE RC
Probe Name Ag5818

| | | | Start | SEQ ID |
|---|---|---|---|---|
| Primers | Sequences | Length | Position | NO: |
| Forward | 5′-ccatcaccttgatcttgtcc-3′ | 20 | 1341 | 444 |
| Probe | TET-5′-ttatgactcctgttttcaccatggaggca-3′-TAMRA | 29 | 1429 | 445 |
| Reverse | 5′-cagaagactccaattatgttca-3′ | 22 | 1458 | 446 |

TABLE RD
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag3326, | Ag3692, | Ag3692, | | Ag3326, | Ag3692, | Ag3692, |
| Tissue | Run | Run | Run | Tissue | Run | Run | Run |
| Name | 210144197 | 211145262 | 224337942 | Name | 210144197 | 211145262 | 224337942 |
| AD 1 | 2.1 | 4.3 | 1.0 | Control | 8.5 | 15.3 | 12.0 |
| Hippo | | | | (Path) 3 | | | |
| | | | | Temporal | | | |
| | | | | Ctx | | | |
| AD 2 | 20.9 | 28.3 | 25.0 | Control | 31.2 | 36.6 | 52.1 |
| Hippo | | | | (Path) 4 | | | |
| | | | | Temporal | | | |
| | | | | Ctx | | | |
| AD 3 | 0.0 | 0.9 | 0.6 | AD 1 | 2.7 | 3.0 | 0.0 |
| Hippo | | | | Occipital | | | |
| | | | | Ctx | | | |
| AD 4 | 2.1 | 7.1 | 2.6 | AD 2 | 0.0 | 0.0 | 0.0 |
| Hippo | | | | Occipital | | | |
| | | | | Ctx | | | |
| | | | | (Missing) | | | |
| AD 5 | 72.7 | 97.9 | 85.3 | AD 3 | 1.5 | 7.2 | 1.3 |
| hippo | | | | Occipital | | | |
| | | | | Ctx | | | |
| AD 6 | 13.7 | 18.3 | 5.5 | AD 4 | 71.7 | 35.6 | 30.6 |
| Hippo | | | | Occipital | | | |
| | | | | Ctx | | | |
| Control 2 | 14.5 | 20.2 | 15.2 | AD 5 | 25.3 | 31.9 | 12.4 |
| Hippo | | | | Occipital | | | |
| | | | | Ctx | | | |
| Control 4 | 11.7 | 7.4 | 5.1 | AD 6 | 17.2 | 19.1 | 11.2 |
| Hippo | | | | Occipital | | | |
| | | | | Ctx | | | |
| Control | 6.7 | 4.4 | 4.5 | Control 1 | 7.0 | 9.0 | 8.1 |
| (Path) 3 | | | | Occipital | | | |
| Hippo | | | | Ctx | | | |
| AD 1 | 4.0 | 1.7 | 2.8 | Control 2 | 33.2 | 44.8 | 26.1 |
| Temporal | | | | Occipital | | | |
| Ctx | | | | Ctx | | | |
| AD 2 | 80.7 | 50.7 | 37.4 | Control 3 | 30.1 | 37.6 | 21.9 |
| Temporal | | | | Occipital | | | |
| Ctx | | | | Ctx | | | |
| AD 3 | 3.6 | 0.0 | 1.1 | Control 4 | 16.3 | 12.6 | 8.2 |
| Temporal | | | | Occipital | | | |
| Ctx | | | | Ctx | | | |
| AD 4 | 19.5 | 30.6 | 15.2 | Control | 42.0 | 55.9 | 52.9 |
| Temporal | | | | (Path) 1 | | | |
| Ctx | | | | Occipital | | | |
| | | | | Ctx | | | |
| AD 5 Inf | 100.0 | 100.0 | 99.3 | Control | 6.7 | 13.0 | 7.7 |
| Temporal | | | | (Path) 2 | | | |
| Ctx | | | | Occipital | | | |
| | | | | Ctx | | | |
| AD 5 | 32.8 | 29.1 | 33.2 | Control | 8.7 | 6.6 | 5.4 |
| SupTemporal | | | | (Path) 3 | | | |
| Ctx | | | | Occipital | | | |
| | | | | Ctx | | | |
| AD 6 Inf | 27.7 | 21.3 | 26.6 | Control | 8.1 | 9.0 | 7.4 |
| Temporal | | | | (Path) 4 | | | |
| Ctx | | | | Occipital | | | |
| | | | | Ctx | | | |
| AD 6 Sup | 41.8 | 53.6 | 17.0 | Control 1 | 21.2 | 23.0 | 15.3 |
| Temporal | | | | Parietal | | | |
| Ctx | | | | Ctx | | | |
| Control 1 | 12.0 | 33.9 | 18.3 | Control 2 | 48.6 | 38.2 | 22.1 |
| Temporal | | | | Parietal | | | |
| Ctx | | | | Ctx | | | |
| Control 2 | 30.1 | 49.3 | 44.4 | Control 3 | 28.3 | 34.4 | 32.8 |
| Temporal | | | | Parietal | | | |
| Ctx | | | | Ctx | | | |
| Control 3 | 38.7 | 39.5 | 33.4 | Control | 78.5 | 97.3 | 100.0 |
| Temporal | | | | (Path) 1 | | | |
| Ctx | | | | Parietal | | | |
| | | | | Ctx | | | |
| Control 4 | 17.6 | 25.2 | 24.1 | Control | 50.7 | 50.7 | 37.9 |
| Temporal | | | | (Path) 2 | | | |
| Ctx | | | | Parietal | | | |
| | | | | Ctx | | | |
| Control | 69.7 | 70.7 | 49.7 | Control | 10.7 | 10.1 | 9.6 |
| (Path) 1 | | | | (Path) 3 | | | |
| Temporal | | | | Parietal | | | |
| Ctx | | | | Ctx | | | |
| Control | 35.4 | 50.7 | 33.4 | Control | 30.6 | 24.5 | 40.9 |
| (Path) 2 | | | | (Path) 4 | | | |
| Temporal | | | | Parietal | | | |
| Ctx | | | | Ctx | | | |

TABLE RE
General_screening_panel_v1.4

| | Rel. Exp. (%) | Rel. Exp. (%) | | Rel. Exp. (%) | Rel. Exp. (%) |
|---|---|---|---|---|---|
| | Ag3326, Run | Ag3692, Run | | Ag3326, Run | Ag3692, Run |
| Tissue Name | 215678613 | 217131191 | Tissue Name | 215678613 | 217131191 |
| Adipose | 0.0 | 0.0 | Renal ca. TK-10 | 11.4 | 12.0 |
| Melanoma* | 0.0 | 0.0 | Bladder | 0.0 | 0.1 |
| Hs688(A).T | | | | | |
| Melanoma* | 0.1 | 0.0 | Gastric ca. (liver | 0.0 | 0.0 |
| Hs688(B).T | | | met.) NCI-N87 | | |
| Melanoma* | 0.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. | 0.0 | 0.0 |
| SK-MEL-5 | | | SW480 | | |
| Squamous | 0.9 | 0.7 | Colon ca.* | 0.0 | 0.0 |
| cell | | | (SW480 met) | | |
| carcinoma | | | SW620 | | |
| SCC-4 | | | | | |
| Testis Pool | 0.1 | 0.2 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 0.0 | 0.0 | Colon ca. CaCo-2 | 0.0 | 0.0 |
| Placenta | 0.0 | 0.0 | Colon cancer | 0.1 | 0.0 |
| | | | tissue | | |
| Uterus Pool | 0.0 | 0.0 | Colon ca. | 0.0 | 0.0 |
| | | | SW1116 | | |
| Ovarian ca. | 0.0 | 0.0 | Colon ca. Colo- | 0.0 | 0.0 |
| OVCAR-3 | | | 205 | | |
| Ovarian ca. | 0.0 | 0.0 | Colon ca. SW-48 | 0.0 | 0.0 |
| SK-OV-3 | | | | | |
| Ovarian ca. | 0.1 | 0.0 | Colon Pool | 0.6 | 0.0 |
| OVCAR-4 | | | | | |
| Ovarian ca. | 0.0 | 0.0 | Small Intestine | 0.1 | 0.0 |
| OVCAR-5 | | | Pool | | |
| Ovarian ca. | 0.0 | 0.0 | Stomach Pool | 0.0 | 0.0 |
| IGROV-1 | | | | | |
| Ovarian ca. | 2.8 | 2.2 | Bone Marrow | 0.0 | 0.1 |
| OVCAR-8 | | | Pool | | |
| Ovary | 0.7 | 0.6 | Fetal Heart | 0.0 | 0.0 |
| Breast ca. | 0.0 | 0.0 | Heart Pool | 0.0 | 0.0 |
| MCF-7 | | | | | |
| Breast ca. | 0.0 | 0.0 | Lymph Node | 0.1 | 0.0 |
| MDA-MB- | | | Pool | | |
| 231 | | | | | |
| Breast ca. BT | 0.6 | 0.8 | Fetal Skeletal | 0.0 | 0.0 |
| 549 | | | Muscle | | |
| Breast ca. | 0.0 | 0.0 | Skeletal Muscle | 0.0 | 0.0 |
| T47D | | | Pool | | |
| Breast ca. | 0.0 | 0.0 | Spleen Pool | 0.4 | 0.2 |
| MDA-N | | | | | |
| Breast Pool | 0.0 | 0.1 | Thymus pool | 0.0 | 0.0 |
| Trachea | 0.2 | 0.1 | CNS cancer | 0.0 | 0.0 |
| | | | (glio/astro) U87- | | |
| | | | MG | | |
| Lung | 0.0 | 0.0 | CNS cancer | 0.0 | 0.0 |
| | | | (glio/astro) U- | | |
| | | | 118-MG | | |
| Fetal Lung | 0.2 | 0.1 | CNS cancer | 0.0 | 0.0 |
| | | | (neuro; met) SK- | | |
| | | | N-AS | | |
| Lung ca. | 0.0 | 0.0 | CNS cancer | 0.0 | 0.0 |
| NCI-N417 | | | (astro) SF-539 | | |
| Lung ca. LX-1 | 0.0 | 0.0 | CNS cancer | 0.0 | 0.0 |
| | | | (astro) SNB-75 | | |
| Lung ca. | 0.0 | 0.0 | CNS cancer | 0.0 | 0.0 |
| NCI-H146 | | | (glio) SNB-19 | | |
| Lung ca. | 0.0 | 0.0 | CNS cancer | 0.1 | 0.1 |
| SHP-77 | | | (glio) SF-295 | | |
| Lung ca. | 0.0 | 0.1 | Brain | 0.4 | 0.4 |
| A549 | | | (Amygdala) Pool | | |
| Lung ca. | 2.0 | 0.0 | Brain | 1.4 | 1.0 |
| NCI-H526 | | | (cerebellum) | | |
| Lung ca. | 0.7 | 0.6 | Brain (fetal) | 0.7 | 0.4 |
| NCI-H23 | | | | | |
| Lung ca. | 0.0 | 0.0 | Brain | 0.5 | 0.7 |
| NCI-H460 | | | (Hippocampus) | | |
| | | | Pool | | |
| Lung ca. | 0.1 | 0.2 | Cerebral Cortex | 1.4 | 1.5 |
| HOP-62 | | | Pool | | |
| Lung ca. | 0.0 | 0.0 | Brain (Substantia | 1.4 | 1.4 |
| NCI-H522 | | | nigra) Pool | | |
| Liver | 28.7 | 24.1 | Brain | 1.1 | 0.9 |
| | | | (Thalamus) Pool | | |
| Fetal Liver | 100.0 | 100.0 | Brain (whole) | 4.1 | 3.7 |
| Liver ca. | 29.5 | 26.2 | Spinal Cord Pool | 0.1 | 0.2 |
| HepG2 | | | | | |
| Kidney Pool | 0.0 | 0.0 | Adrenal Gland | 2.6 | 1.9 |
| Fetal Kidney | 0.1 | 0.1 | Pituitary gland | 0.0 | 0.2 |
| | | | Pool | | |
| Renal ca. | 0.0 | 0.0 | Salivary Gland | 40.9 | 35.1 |
| 786-0 | | | | | |
| Renal ca. | 0.0 | 0.0 | Thyroid (female) | 0.0 | 0.0 |
| A498 | | | | | |
| Renal ca. | 0.0 | 0.0 | Pancreatic ca. | 0.5 | 0.8 |
| ACHN | | | CAPAN2 | | |
| Renal ca. | 0.0 | 0.0 | Pancreas Pool | 0.0 | 0.0 |
| UO-31 | | | | | |

TABLE RF
General_screening_panel_v1.5

| | Rel. Exp. (%) | | Rel. Exp. (%) |
|---|---|---|---|
| | Ag5818, Run | | Ag5818, Run |
| Tissue Name | 245382899 | Tissue Name | 245382899 |
| Adipose | 0.0 | Renal ca. TK-10 | 13.4 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 1.4 | Colon ca.* (SW480 | 0.0 |
| carcinoma SCC-4 | | met) SW620 | |
| Testis Pool | 0.5 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.4 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.1 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 1.9 | Bone Marrow Pool | 0.0 |
| OVCAR-8 | | | |
| Ovary | 0.3 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.4 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.7 |
| Breast Pool | 0.0 | Thymus Pool | 0.0 |
| Trachea | 0.2 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.2 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.2 | Brain (Amygdala) Pool | 0.7 |
| Lung ca. NCI-H526 | 0.0 | Brain (cerebellum) | 1.1 |
| Lung ca. NCI-H23 | 1.5 | Brain (fetal) | 0.8 |
| Lung ca. NCI-H460 | 0.0 | Brain (Hippocampus) | 0.6 |
| | | Pool | |
| Lung ca. HOP-62 | 0.0 | Cerebral Cortex Pool | 1.7 |
| Lung ca. NCI-H522 | 0.0 | Brain (Substantia | 1.2 |
| | | nigra) Pool | |
| Liver | 40.3 | Brain (Thalamus) Pool | 1.3 |
| Fetal Liver | 100.0 | Brain (whole) | 5.6 |
| Liver ca. HepG2 | 33.2 | Spinal Cord Pool | 0.3 |
| Kidney Pool | 0.0 | Adrenal Gland | 6.0 |
| Fetal Kidney | 0.0 | Pituitary gland Pool | 0.2 |
| Renal ca. 786-0 | 0.0 | Salivary Gland | 67.4 |
| Renal ca. A498 | 0.0 | Thyroid (female) | 0.0 |
| Renal ca. ACHN | 0.0 | Pancreatic ca. | 0.7 |
| | | CAPAN2 | |
| Renal ca. UO-31 | 0.0 | Pancreas Pool | 0.0 |

TABLE RG
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3692, | Ag5818, | | Ag3692, | Ag5818, |
| | Run | Run | | Run | Run |
| Tissue Name | 169987356 | 246920287 | Tissue Name | 169987356 | 246920287 |
| Secondary Th1 act | 0.0 | 0.0 | HUVEC IL-1beta | 0.0 | 0.0 |
| Secondary Th2 act | 0.0 | 0.0 | HUVEC IFN | 0.0 | 0.0 |
| | | | gamma | | |
| Secondary Tr1 act | 0.0 | 0.0 | HUVEC TNF | 0.0 | 0.0 |
| | | | alpha + IFN | | |
| | | | gamma | | |
| Secondary Th1 rest | 0.0 | 0.0 | HUVEC TNF | 0.0 | 0.0 |
| | | | alpha + IL4 | | |
| Secondary Th2 rest | 0.0 | 0.0 | HUVEC IL-11 | 0.0 | 0.0 |
| Secondary Tr1 rest | 0.0 | 0.0 | Lung | 0.0 | 0.0 |
| | | | Microvascular EC | | |
| | | | none | | |
| Primary Th1 act | 0.0 | 0.0 | Lung | 0.0 | 0.0 |
| | | | Microvascular EC | | |
| | | | TNF alpha + IL- | | |
| | | | 1beta | | |
| Primary Th2 act | 0.0 | 0.0 | Microvascular | 11.3 | 0.0 |
| | | | Dermal EC none | | |
| Primary Tr1 act | 4.2 | 0.0 | Microsvasular | 0.0 | 0.0 |
| | | | Dermal EC | | |
| | | | TNF alpha + IL- | | |
| | | | 1beta | | |
| Primary Th1 rest | 0.0 | 0.0 | Bronchial | 28.5 | 0.0 |
| | | | epithelium | | |
| | | | TNF alpha + | | |
| | | | IL1beta | | |
| Primary Th2 rest | 0.0 | 0.0 | Small airway | 5.7 | 0.0 |
| | | | epithelium none | | |
| Primary Tr1 rest | 0.0 | 0.0 | Small airway | 0.0 | 0.0 |
| | | | epithelium | | |
| | | | TNF alpha + IL- | | |
| | | | 1beta | | |
| CD45RA CD4 | 3.9 | 0.0 | Coronery artery | 0.0 | 0.0 |
| lymphocyte act | | | SMC rest | | |
| CD45RO CD4 | 0.0 | 0.0 | Coronery artery | 0.0 | 0.0 |
| lymphocyte act | | | SMC TNF alpha + | | |
| | | | IL-1beta | | |
| CD8 lymphocyte | 0.0 | 0.0 | Astrocytes rest | 0.0 | 0.0 |
| act | | | | | |
| Secondary CD8 | 0.0 | 0.0 | Astrocytes | 0.0 | 0.0 |
| lymphocyte rest | | | TNF alpha + IL- | | |
| | | | 1beta | | |
| Secondary CD8 | 0.0 | 0.0 | KU-812 | 3.6 | 24.3 |
| lymphocyte act | | | (Basophil) rest | | |
| CD4 lymphocyte | 0.0 | 0.0 | KU-812 | 4.3 | 0.0 |
| none | | | (Basophil) | | |
| | | | PMA/ionomycin | | |
| 2ry | 0.0 | 0.0 | CCD1106 | 10.7 | 0.0 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.0 |
| | | | (Keratinocytes) | | |
| | | | TNF alpha + IL- | | |
| | | | 1beta | | |
| LAK cells IL-2 | 0.0 | 0.0 | Liver cirrhosis | 94.0 | 27.5 |
| LAK cells IL- | 0.0 | 0.0 | NCI-H292 none | 0.0 | 0.0 |
| 2 + IL-12 | | | | | |
| LAK cells IL- | 0.0 | 0.0 | NCI-H292 IL-4 | 0.0 | 0.0 |
| 2 + IFN gamma | | | | | |
| LAK cells IL-2 + | 0.0 | 0.0 | NCI-H292 IL-9 | 0.0 | 0.0 |
| IL-18 | | | | | |
| LAK cells | 0.0 | 0.0 | NCI-H292 IL-13 | 0.0 | 0.0 |
| PMA/ionomycin | | | | | |
| NK Cells IL-2 rest | 0.0 | 0.0 | NCI-H292 IFN | 0.0 | 0.0 |
| | | | gamma | | |
| Two Way MLR 3 | 0.0 | 0.0 | HPAEC none | 0.0 | 0.0 |
| day | | | | | |
| Two Way MLR 5 | 3.2 | 0.0 | HPAEC TNF | 0.0 | 0.0 |
| day | | | alpha + IL-1beta | | |
| Two Way MLR 7 | 0.0 | 0.0 | Lung fibroblast | 0.0 | 0.0 |
| day | | | none | | |
| PBMC rest | 0.0 | 0.0 | Lung fibroblast | 0.0 | 0.0 |
| | | | TNF alpha + IL- | | |
| | | | 1beta | | |
| PBMC PWM | 0.0 | 0.0 | Lung fibroblast | 0.0 | 0.0 |
| | | | IL-4 | | |
| PBMC PHA-L | 0.0 | 0.0 | Lung fibroblast | 0.0 | 0.0 |
| | | | IL-9 | | |
| Ramos (B cell) | 0.0 | 0.0 | Lung fibroblast | 0.0 | 0.0 |
| none | | | IL-13 | | |
| Ramos (B cell) | 0.0 | 0.0 | Lung fibroblast | 0.0 | 0.0 |
| ionomycin | | | IFN gamma | | |
| B lymphocytes | 0.0 | 0.0 | Dermal fibroblast | 0.0 | 0.0 |
| PWM | | | CCD1070 rest | | |
| B lymphocytes | 0.0 | 0.0 | Dermal fibroblast | 0.0 | 0.0 |
| CD40L and IL-4 | | | CCD1070 TNF | | |
| | | | alpha | | |
| EOL-1 dbcAMP | 0.0 | 0.0 | Dermal fibroblast | 0.0 | 0.0 |
| | | | CCD1070 IL- | | |
| | | | 1beta | | |
| EOL-1 dbcAMP | 0.0 | 0.0 | Dermal fibroblast | 0.0 | 0.0 |
| PMA/ionomycin | | | IFN gamma | | |
| Dendritic cells | 0.0 | 0.0 | Dermal fibroblast | 0.0 | 0.0 |
| none | | | IL-4 | | |
| Dendritic cells LPS | 0.0 | 0.0 | Dermal | 0.0 | 0.0 |
| | | | Fibroblasts rest | | |
| Dendritic cells | 0.0 | 0.0 | Neutrophils | 0.0 | 0.0 |
| anti-CD40 | | | TNFa + LPS | | |
| Monocytes rest | 0.0 | 0.0 | Neutrophils rest | 0.0 | 0.0 |
| Monocytes LPS | 0.0 | 0.0 | Colon | 0.0 | 0.0 |
| Macrophages rest | 0.0 | 0.0 | Lung | 0.0 | 0.0 |
| Macrophages LPS | 0.0 | 0.0 | Thymus | 2.4 | 0.0 |
| HUVEC none | 0.0 | 0.0 | Kidney | 100.0 | 100.0 |
| HUVEC starved | 0.0 | 0.0 | | | |

TABLE RH
Panel 5 Islet

| | Rel. Exp. (%) | | Rel. Exp. (%) |
|---|---|---|---|
| | Ag3326, Run | | Ag3326, Run |
| Tissue Name | 242385365 | Tissue Name | 242385365 |
| 97457_Patient- | 0.0 | 94709_Donor 2 AM - A_adipose | 0.2 |
| 02go_adipose | | | |
| 97476_Patient- | 0.0 | 94710_Donor 2 AM - B_adipose | 0.0 |
| 07sk_skeletal muscle | | | |
| 97477_Patient- | 0.0 | 94711_Donor 2 AM - C_adipose | 0.0 |
| 07ut_uterus | | | |
| 97478_Patient- | 0.0 | 94712_Donor 2 AD - A_adipose | 0.0 |
| 07pl_placenta | | | |
| 99167_Bayer Patient | 0.3 | 94713_Donor 2 AD - B_adipose | 0.0 |
| 1 | | | |
| 97482_Patient- | 0.0 | 94714_Donor 2 AD - C_adipose | 0.0 |
| 08ut_uterus | | | |
| 97483_Patient- | 0.0 | 94742_Donor 3 U - | 0.0 |
| 08pl_placenta | | A_Mesenchymal Stem Cells | |
| 97486_Patient- | 0.0 | 94743_Donor 3 U - | 0.0 |
| 09sk_skeletal muscle | | B_Mesenchymal Stem Cells | |
| 97487_Patient- | 0.0 | 94730_Donor 3 AM - A_adipose | 0.0 |
| 09ut_uterus | | | |
| 97488_Patient- | 0.0 | 94731_Donor 3 AM - B_adipose | 0.0 |
| 09pl_placenta | | | |
| 97492_Patient- | 0.0 | 94732_Donor 3 AM - C_adipose | 0.0 |
| 10ut_uterus | | | |
| 97493_Patient- | 0.0 | 94733_Donor 3 AD - A_adipose | 0.0 |
| 10pl_placenta | | | |
| 97495_Patient- | 0.0 | 94734_Donor 3 AD - B_adipose | 0.0 |
| 11go_adipose | | | |
| 97496_Patient- | 0.0 | 94735_Donor 3 AD - C_adipose | 0.0 |
| 11sk_skeletal muscle | | | |
| 97497_Patient- | 0.0 | 77138_Liver_HepG2untreated | 100.0 |
| 11ut_uterus | | | |
| 97498_Patient- | 0.0 | 73556_Heart_Cardiac stromal | 0.0 |
| 11pl_placenta | | cells (primary) | |
| 97500_Patient- | 0.1 | 81735_Small Intestine | 39.5 |
| 12go_adipose | | | |
| 97501_Patient- | 0.3 | 72409_Kidney_Proximal | 0.0 |
| 12sk_skeletal muscle | | Convoluted Tubule | |
| 97502_Patient- | 0.0 | 82685_Small | 0.0 |
| 12ut_uterus | | intestine_Duodenum | |
| 97503_Patient- | 0.0 | 90650_Adrenal_Adrenocortical | 0.0 |
| 12pl_placenta | | adenoma | |
| 94721_Donor 2 U - | 0.0 | 72410_Kidney_HRCE | 0.0 |
| A_Mesenchymal | | | |
| Stem Cells | | | |
| 94722_Donor 2 U - | 0.0 | 72411_Kidney_HRE | 0.0 |
| B_Mesenchymal | | | |
| Stem Cells | | | |
| 94723_Donor 2 U - | 0.0 | 73139_Uterus_Uterine smooth | 0.0 |
| C_Mesenchymal | | muscle cells | |
| Stem Cells | | | |

CNS_neurodegeneration_v1.0 Summary: Ag3326/Ag3692—Three experiments done with two primer pairs (same sequence) are in excellent agreement. This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders. Ag5818 Results from one experiment are not included. The amp plot indicates that there were experimental difficulties with this run.

General_screening_panel_v1.4 Summary: Ag3326/Ag3692 Two experiments with the same probe and primer set produce results that are in excellent agreement. This gene is highly expressed in fetal liver (CT=26.5-27.0) and moderately expressed in adult liver (CT=28.5-28.8) and liver cancer cell line HepG2 (CT=28.4-28.8). This result agrees with In the results seen in Panel 5 (expression in HepG2 (CT=29.2). These results are in agreement with published data that show a novel sodium dicarboxylate transporter in brain, choroid plexus kidney, intestine and liver. Thus, expression of this gene could be used to differentiate between these samples and other samples on this panel and as a marker for liver derived tissue.

This gene is expressed at low levels throughout the CNS, including in amygdala, substantia nigra, thalamus, cerebellum, and cerebral cortex. Therefore, this gene may play a role in central nervous system disorders such as Parkinson's disease, epilepsy, multiple sclerosis, schizophrenia and depression.

Low but significant levels of expression are also seen in the adrenal gland. Thus, this gene product may also be involved in metabolic disorders of this gland, including adrenoleukodystrophy and congenital adrenal hyperplasia.

### REFERENCES

1. Pajor A M, Gangula R, Yao X. Cloning and functional characterization of a high-affinity Na(+)/dicarboxylate cotransporter from mouse brain. Am J Physiol Cell Physiol May 2001;280(5):C1215-23.

2. Chen X Z, Shayakul C, Berger UV, Tian W, Hediger M A. Characterization of a rat Na+-dicarboxylate cotransporter. J Biol Chem Aug. 14, 1998;273(33):20972-81.

General_screening_panel_v1.5 Summary: Ag5818 Results using this primer pair are in excellent agreement with the results seen in panel 1.4. See Panel 1.4 for discussion. Panel 4.1D Summary: Ag3692 Significant expression of this gene is seen only in kidney and a liver cirrhosis sample (CTs=34.0). These results confirm that this gene is expressed in liver derived samples. The presence in the kidney is also in agreement with published results. Please see Panel 1.4. This gene product may be involved in maintaining or restoring normal function to the kidney during inflammation.

Panel 4D Summary: Ag3326 Results from one experiment are not included. The amp plot indicates that there were experimental difficulties with this run.

Panel 5 Islet Summary: Ag3326 The highest expression of this gene is in liver cancer cell line HepG2 (CT=29.2). There is also moderate expression in the small intestine (CT=30.5). These results compare well with previously published reports of sodium dicarboxylate transporter expression in mouse and rat (see discussion Panel 1.4).

S. CG57732-01 and CG57732-02 and CG57732-03: CA2+/Calmodulin-Dependent Protein Kinase IV Kinase

Expression of gene CG57732-01 and full length clones CG57732-02 and CG57732-03, was assessed using the primer-probe set Ag3317, described in Table SA. Results of the RTQ-PCR runs are shown in Tables SB, SC and SD. Please note CG57732-03 represents a splice variant of CG57732-01.

Table SA. Probe Name Ag3317

TABLE SA
Probe Name Ag3317

| | | | Start | SEQ ID |
|---|---|---|---|---|
| Primers | Sequences | Length | Position | NO: |
| Forward | 5′-ggcctacaacgaaagtgaaga-3′ | 21 | 451 | 447 |
| Probe | TET-5′-cagacactatgcaatgaaagtcctttcca-3′-TAMRA | 29 | 472 | 448 |
| Reverse | 5′-ggaaagccatactgcttcagta-3′ | 22 | 510 | 449 |

TABLE SB
CNS_neurodegeneration_v1.0

| | | | Rel. Exp. |
|---|---|---|---|
| | Rel. Exp. (%) | | (%) Ag3317, |
| | Ag3317, Run | | Run |
| Tissue Name | 210144081 | Tissue Name | 210144081 |
| AD 1 Hippo | 10.7 | Control (Path) 3 | 4.1 |
| | | Temporal Ctx | |
| AD 2 Hippo | 23.7 | Control (Path) 4 | 42.6 |
| | | Temporal Ctx | |
| AD 3 Hippo | 4.5 | AD 1 Occipital | 12.5 |
| | | Ctx | |
| AD 4 Hippo | 7.5 | AD 2 Occipital | 0.0 |
| | | Ctx (Missing) | |
| AD 5 hippo | 97.9 | AD 3 Occipital | 5.4 |
| | | Ctx | |
| AD 6 Hippo | 25.7 | AD 4 Occipital | 18.4 |
| | | Ctx | |
| Control 2 Hippo | 24.8 | AD 5 Occipital | 21.8 |
| | | Ctx | |
| Control 4 Hippo | 4.3 | AD 6 Occipital | 58.6 |
| | | Ctx | |
| Control (Path) 3 | 2.8 | Control 1 Occipital | 1.5 |
| Hippo | | Ctx | |
| AD 1 Temporal Ctx | 10.4 | Control 2 Occipital | 94.0 |
| | | Ctx | |
| AD 2 Temporal Ctx | 35.8 | Control 3 Occipital | 21.5 |
| | | Ctx | |
| AD 3 Temporal Ctx | 5.8 | Control 4 Occipital | 2.6 |
| | | Ctx | |
| AD 4 Temporal Ctx | 23.2 | Control (Path) 1 | 100.0 |
| | | Occipital Ctx | |
| AD 5 Inf Temporal | 88.9 | Control (Path) 2 | 13.8 |
| Ctx | | Occipital Ctx | |
| AD 5 Sup Temporal | 26.6 | Control (Path) 3 | 0.9 |
| Ctx | | Occipital Ctx | |
| AD 6 Inf Temporal | 39.5 | Control (Path) 4 | 19.6 |
| Ctx | | Occipital Ctx | |
| AD 6 Sup Temporal | 47.3 | Control 1 Parietal | 4.9 |
| Ctx | | Ctx | |
| Control 1 Temporal | 4.4 | Control 2 Parietal | 33.0 |
| Ctx | | Ctx | |
| Control 2 Temporal | 63.3 | Control 3 Parietal | 27.4 |
| Ctx | | Ctx | |
| Control 3 Temporal | 20.4 | Control (Path) 1 | 95.9 |
| Ctx | | Parietal Ctx | |
| Control 4 Temporal | 8.7 | Control (Path) 2 | 24.5 |
| Ctx | | Parietal Ctx | |
| Control (Path) 1 | 77.4 | Control (Path) 3 | 2.0 |
| Temporal Ctx | | Parietal Ctx | |
| Control (Path) 2 | 38.7 | Control (Path) 4 | 51.8 |
| Temporal Ctx | | Parietal Ctx | |

TABLE SC
General_screening_panel_v1.4

| | Rel. Exp. | | Rel. Exp. |
|---|---|---|---|
| | (%) Ag3317, | | (%) Ag3317, |
| | Run | | Run |
| Tissue Name | 215678602 | Tissue Name | 215678602 |
| Adipose | 2.4 | Renal ca. TK-10 | 14.2 |
| Melanoma* | 6.2 | Bladder | 10.5 |
| Hs688(A).T | | | |
| Melanoma* | 7.9 | Gastric ca. (liver met) | 22.2 |
| Hs688(B).T | | NCI-N87 | |
| Melanoma* M14 | 18.2 | Gastric ca. KATO | 23.0 |
| | | III | |
| Melanoma* | 9.4 | Colon ca. SW-948 | 11.1 |
| LOXIMVI | | | |
| Melanoma* SK- | 9.8 | Colon ca. SW480 | 20.9 |
| MEL-5 | | | |
| Squamous cell | 1.6 | Colon ca.* (SW | 21.6 |
| carcinoma SCC-4 | | 480 met) SW620 | |
| Testis Pool | 13.1 | Colon ca. HT29 | 11.3 |
| Prostate ca.* (bone | 6.4 | Colon ca. HCT-116 | 27.0 |
| met) PC-3 | | | |
| Prostate Pool | 3.1 | Colon ca. CaCo-2 | 1.6 |
| Placenta | 1.8 | Colon cancer tissue | 11.3 |
| Uterus Pool | 3.9 | Colon ca. SW1116 | 9.7 |
| Ovarian ca. | 11.6 | Colon ca. Colo-205 | 1.7 |
| OVCAR-3 | | | |
| Ovarian ca. SK- | 18.7 | Colon ca. SW-48 | 8.8 |
| OV-3 | | | |
| Ovarian ca. | 3.4 | Colon Pool | 17.1 |
| OVCAR-4 | | | |
| Ovarian ca. | 17.2 | Small Intestine Pool | 21.2 |
| OVCAR-5 | | | |
| Ovarian ca. | 6.2 | Stomach Pool | 5.3 |
| IGROV-1 | | | |
| Ovarian ca. | 4.7 | Bone Marrow Pool | 5.1 |
| OVCAR-8 | | | |
| Ovary | 2.9 | Fetal Heart | 6.8 |
| Breast ca. MCF-7 | 6.1 | Heart Pool | 5.4 |
| Breast ca. MDA- | 20.3 | Lymph Node Pool | 13.4 |
| MB-231 | | | |
| Breast ca. BT 549 | 7.4 | Fetal Skeletal Muscle | 2.6 |
| Breast ca. T47D | 37.9 | Skeletal Muscle Pool | 2.3 |
| Breast ca. MDA-N | 9.0 | Spleen Pool | 2.8 |
| Breast Pool | 12.0 | Thymus Pool | 9.0 |
| Trachea | 17.2 | CNS cancer | 66.4 |
| | | (glio/astro) U87-MG | |
| Lung | 0.7 | CNS cancer | 53.2 |
| | | (glio/astro) U-118-MG | |
| Fetal Lung | 6.0 | CNS cancer | 4.6 |
| | | (neuro; met) SK-N-AS | |
| Lung ca. NCI-N417 | 16.5 | CNS cancer (astro) | 17.2 |
| | | SF-539 | |
| Lung ca. LX-1 | 20.9 | CNS cancer (astro) | 21.5 |
| | | SNB-75 | |
| Lung ca. NCI-H146 | 7.0 | CNS cancer (glio) | 5.1 |
| | | SNB-19 | |
| Lung ca. SHP-77 | 23.0 | CNS cancer (glio) | 12.2 |
| | | SF-295 | |
| Lung ca. A549 | 23.7 | Brain (Amygdala) | 46.3 |
| | | Pool | |
| Lung ca. NCI-H526 | 4.4 | Brain (Cerebellum) | 92.7 |
| Lung ca. NCI-H23 | 5.8 | Brain (fetal) | 25.7 |
| Lung ca. NCI-H460 | 10.3 | Brain (Hippocampus) | 42.9 |
| | | Pool | |
| Lung ca. HOP-62 | 7.0 | Cerebral Cortex Pool | 100.0 |
| Lung ca. NCI-H522 | 2.9 | Brain (Substantia | 76.3 |
| | | nigra) Pool | |
| Liver | 0.1 | Brain (Thalamus) Pool | 63.7 |
| Fetal Liver | 1.3 | Brain (whole) | 56.6 |
| Liver ca. HepG2 | 1.4 | Spinal Cord Pool | 9.3 |
| Kidney Pool | 26.2 | Adrenal Gland | 16.2 |
| Fetal Kidney | 3.5 | Pituitary gland Pool | 16.4 |
| Renal ca. 786-0 | 26.4 | Salivary Gland | 13.4 |
| Renal ca. A498 | 14.2 | Thyroid (female) | 6.3 |
| Renal ca. ACHN | 33.2 | Pancreatic ca. | 2.6 |
| | | CAPAN2 | |
| Renal ca. UO-31 | 4.3 | Pancreas Pool | 19.6 |

TABLE SD
Panel 4D

| | Rel. Exp. (%) | | Rel. Exp. (%) |
|---|---|---|---|
| | Ag3317, Run | | Ag3317, Run |
| Tissue Name | 164683049 | Tissue Name | 164683049 |
| Secondary Th1 act | 21.6 | HUVEC IL-1beta | 3.8 |
| Secondary Th2 act | 23.2 | HUVEC IFN gamma | 12.5 |
| Secondary Tr1 act | 22.8 | HUVEC TNF alpha + | 2.9 |
| | | IFN gamma | |
| Secondary Th1 rest | 12.7 | HUVEC TNF alpha + | 9.0 |
| | | IL4 | |
| Secondary Th2 rest | 9.3 | HUVEC IL-11 | 4.0 |
| Secondary Tr1 rest | 33.7 | Lung Microvascular EC | 24.3 |
| | | none | |
| Primary Th1 act | 44.1 | Lung Microvascular EC | 11.3 |
| | | TNF alpha + IL-1beta | |
| Primary Th2 act | 49.3 | Microvascular Dermal | 41.5 |
| | | none | |
| Primary Tr1 act | 74.2 | Microsvasular Dermal EC | 17.2 |
| | | TNF alpha + IL-1beta | |
| Primary Th1 rest | 38.2 | Bronchial epithelium | 31.2 |
| | | TNF alpha + IL1beta | |
| Primary Th2 rest | 44.4 | Small airway epithelium | 8.0 |
| | | none | |
| Primary Tr1 rest | 50.0 | Small airway epithelium | 11.1 |
| | | TNF alpha + IL-1beta | |
| CD45RA CD4 | 41.2 | Coronery artery SMC rest | 20.6 |
| lymphocyte act | | | |
| CD45RO CD4 | 25.0 | Coronery artery SMC | 19.6 |
| lymphocyte act | | TNF alpha + IL-1beta | |
| CD8 lymphocyte act | 17.8 | Astrocytes rest | 14.7 |
| Secondary CD8 | 21.8 | Astrocytes TNF alpha + | 11.0 |
| lymphocyte rest | | IL-1beta | |
| Secondary CD8 | 7.4 | KU-812 (Basophil) rest | 2.1 |
| lymphocyte act | | | |
| CD4 lymphocyte none | 21.8 | KU-812 (Basophil) | 12.9 |
| | | PMA/ionomycin | |
| 2ry Th1/Th2/Tr1_anti- | 5.8 | CCD1106 | 30.6 |
| CD95 CH11 | | (Keratinocytes) none | |
| LAK cells rest | 51.1 | CCD1106 | 23.7 |
| | | (Keratinocytes) | |
| | | TNF alpha + IL-1beta | |
| LAK cells IL-2 | 7.0 | Liver cirrhosis | 0.8 |
| LAK cells IL-2 + IL-12 | 25.5 | Lupus kidney | 2.2 |
| LAK cells IL-2 + IFN | 35.4 | NCI-H292 none | 33.7 |
| gamma | | | |
| LAK cells IL-2 + IL-18 | 28.7 | NCI-H292 IL-4 | 43.5 |
| LAK cells | 20.6 | NCI-H292 IL-9 | 36.3 |
| PMA/ionomycin | | | |
| NK Cells IL-2 rest | 13.5 | NCI-H292 IL-13 | 35.6 |
| Two Way MLR 3 day | 33.0 | NCI-H292 IFN gamma | 24.3 |
| Two Way MLR 5 day | 9.6 | HPAEC none | 22.8 |
| Two Way MLR 7 day | 10.0 | HPAEC TNF alpha + IL- | 8.3 |
| | | 1beta | |
| PBMC rest | 12.0 | Lung fibroblast none | 11.8 |
| PBMC PWM | 24.7 | Lung fibroblast TNF | 1.2 |
| | | alpha + IL-1beta | |
| PBMC PHA-L | 32.5 | Lung fibroblast IL-4 | 19.2 |
| Ramos (B cell) none | 1.5 | Lung fibroblast IL-9 | 12.1 |
| Ramos (B cell) | 0.0 | Lung fibroblast IL-13 | 14.8 |
| ionomycin | | | |
| B lymphocytes PWM | 41.2 | Lung fibroblast IFN | 17.2 |
| | | gamma | |
| B lymphocytes CD40L | 14.5 | Dermal fibroblast | 100.0 |
| and IL-4 | | CCD1070 rest | |
| EOL-1 dbcAMP | 20.0 | Dermal fibroblast | 57.8 |
| | | CCD1070 TNF alpha | |
| EOL-1 dbcAMP | 60.3 | Dermal fibroblast | 14.2 |
| PMA/ionomycin | | CCD1070 IL-1beta | |
| Dendritic cells none | 55.5 | Dermal fibroblast IFN | 24.1 |
| | | gamma | |
| Dendritic cells LPS | 26.1 | Dermal fibroblast IL-4 | 39.0 |
| Dendritic cells anti- | 74.7 | IBD Colitis 2 | 1.6 |
| CD40 | | | |
| Monocytes rest | 48.0 | IBD Crohn's | 2.7 |
| Monocytes LPS | 15.4 | Colon | 19.1 |
| Macrophages rest | 98.6 | Lung | 14.4 |
| Macrophages LPS | 5.6 | Thymus | 10.5 |
| HUVEC none | 27.9 | Kidney | 100.0 |
| HUVEC starved | 27.0 | | |

CNS_neurodegeneration_v1.0 Summary: Ag3317—This panel does not show differential expression of this gene in Alzheimer's disease. However, this expression profile confirms the presence of this gene in the brain. Please see Panel 1.4 for discussion of utility of this gene in the central nervous system.

General_screening_panel_v1.4 Summary: Ag3317—There is low to moderate expression this gene across all samples on this panel. This gene is expressed at moderate levels throughout the CNS, including in amygdala, substantia nigra, thalamus, cerebellum, and cerebral cortex. Highest expression is observed in the cerebral cortex (CT=29.0). This gene encodes a calmodulin-dependent protein kinase IV homolog, which is known to play a role in. Ca2+ signaling in the CNS that controls neuronal growth, differentiation, and plasticity. Mice deficient in calmodulin-dependent protein kinase IV were found to have cerebellar defects. Therefore, this gene may play a role in central nervous system disorders such as Parkinson's disease, epilepsy, multiple sclerosis, schizophrenia and depression.

This gene product is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

Based on expression in this panel, this gene may be also be involved in gastric, pancreatic, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

### REFERENCES

1. Okuno S, Kitani T, Fujisawa H. Evidence for the existence of Ca2+/calmodulin-dependent protein kinase IV kinase isoforms in rat brain. J Biochem (Tokyo) June 1996;119(6):1176-81.

2. Ribar T J, Rodriguiz R M, Khiroug L, Wetsel W C, Augustine G J, Means A R. Cerebellar defects in Ca2+/calmodulin kinase IV-deficient mice. J Neurosci Nov. 15, 2000;20(2C2):RC 107.

Panel 4D Summary: Ag3317—This gene was found to have low expression across almost all the samples on this panel, with the highest level of expression seen in kidney and resting dermal Fibroblasts (CTs=32). Expression of Ca2+/calmodulin-dependent kinase type IV in thymocytes has been found in mice, where it plays a role in Ca2+-dependent gene transcription.

REFERENCE

1. Raman V, Blaeser F, Ho N, Engle D L, Williams C B, Chatila T A. Requirement for Ca2+/calmodulin-dependent kinase type IV/Gr in setting the thymocyte selection threshold. J Immunol Dec. 1, 2001;167(11):6270-8.

T. CG57709-01: Novel Mitochondrial Protein

Expression of gene CG57709-01 was assessed using the primer-probe set Ag3323, described in Table TA. Results of the RTQ-PCR runs are shown in Tables TB, TC and TD.

Table TA. Probe Name Ag3323

TABLE TA
Probe Name Ag3323

| | | | Start | SEQ ID |
|---|---|---|---|---|
| Primers | Sequences | Length | Position | NO: |
| Forward | 5′-atgtgcagaggatacgcatg-3′ | 20 | 589 | 450 |
| Probe | TET-5′-tgcaaaacaggaagacaaaggaaggg-3′-TAMRA | 26 | 626 | 451 |
| Reverse | 5′-tggttctggcattctagacg-3′ | 20 | 665 | 452 |

TABLE TB
CNS_neurodegeneration_v1.0

| | Rel. Exp. | | Rel. Exp. |
|---|---|---|---|
| | (%) Ag3323, | | (%) Ag3323, |
| | Run | | Run |
| Tissue Name | 210144152 | Tissue Name | 210144152 |
| AD 1 Hippo | 22.5 | Control (Path) 3 | 5.2 |
| | | Temporal Ctx | |
| AD 2 Hippo | 29.5 | Control (Path) 4 | 32.5 |
| | | Temporal Ctx | |
| AD 3 Hippo | 6.9 | AD 1 Occipital | 18.6 |
| | | Ctx | |
| AD 4 Hippo | 7.4 | AD 2 Occipital | 0.0 |
| | | Ctx (Missing) | |
| AD 5 hippo | 82.4 | AD 3 Occipital | 7.6 |
| | | Ctx | |
| AD 6 Hippo | 66.4 | AD 4 Occipital | 17.8 |
| | | Ctx | |
| Control 2 Hippo | 27.5 | AD 5 Occipital | 30.8 |
| | | Ctx | |
| Control 4 Hippo | 11.9 | AD 6 Occipital | 48.6 |
| | | Ctx | |
| Control (Path) 3 | 8.4 | Control 1 Occipital | 4.0 |
| Hippo | | Ctx | |
| AD 1 Temporal Ctx | 18.6 | Control 2 Occipital | 58.2 |
| | | Ctx | |
| AD 2 Temporal Ctx | 30.6 | Control 3 Occipital | 14.2 |
| | | Ctx | |
| AD 3 Temporal Ctx | 7.6 | Control 4 Occipital | 6.6 |
| | | Ctx | |
| AD 4 Temporal Ctx | 21.5 | Control (Path) 1 | 70.7 |
| | | Occipital Ctx | |
| AD 5 Inf Temporal | 100.0 | Control (Path) 2 | 12.6 |
| Ctx | | Occipital Ctx | |
| AD 5 Sup Temporal | 42.6 | Control (Path) 3 | 2.4 |
| Ctx | | Occipital Ctx | |
| AD 6 Inf Temporal | 48.6 | Control (Path) 4 | 14.6 |
| Ctx | | Occipital Ctx | |
| AD 6 Sup Temporal | 42.0 | Control 1 Parietal | 6.5 |
| Ctx | | Ctx | |
| Control 1 Temporal | 6.3 | Control 2 Parietal | 48.0 |
| Ctx | | Ctx | |
| Control 2 Temporal | 39.0 | Control 3 Parietal | 19.6 |
| Ctx | | Ctx | |
| Control 3 Temporal | 13.1 | Control (Path) 1 | 61.1 |
| Ctx | | Parietal Ctx | |
| Control 4 Temporal | 8.9 | Control (Path) 2 | 19.3 |
| Ctx | | Parietal Ctx | |
| Control (Path) 1 | 53.6 | Control (Path) 3 | 3.8 |
| Temporal Ctx | | Parietal Ctx | |
| Control (Path) 2 | 34.2 | Control (Path) 4 | 42.6 |
| Temporal Ctx | | Parietal Ctx | |

TABLE TC
Panel 1.3D

| | Rel. Exp. (%) | | Rel. Exp. (%) |
|---|---|---|---|
| | Ag3323, | | Ag3323, |
| Tissue Name | Run 165678151 | Tissue Name | Run 165678151 |
| Liver adenocarcinoma | 25.0 | Kidney (fetal) | 6.5 |
| Pancreas | 12.8 | Renal ca. 786-0 | 14.3 |
| Pancreatic ca. CAPAN 2 | 24.5 | Renal ca. A498 | 34.2 |
| Adrenal gland | 12.2 | Renal ca. RXF 393 | 14.2 |
| Thyroid | 6.9 | Renal ca. ACHN | 12.9 |
| Salivary gland | 14.0 | Renal ca. UO-31 | 48.6 |
| Pituitary gland | 10.1 | Renal ca. TK-10 | 7.2 |
| Brain (fetal) | 13.7 | Liver | 20.2 |
| Brain (whole) | 29.7 | Liver (fetal) | 22.1 |
| Brain (amygdala) | 21.3 | Liver ca. | 21.3 |
| | | (hepatoblast) HepG2 | |
| Brain (cerebellum) | 24.7 | Lung | 6.7 |
| Brain (hippocampus) | 25.7 | Lung (fetal) | 14.8 |
| Brain (substantia nigra) | 20.0 | Lung ca. (small cell) | 39.8 |
| | | LX-1 | |
| Brain (thalamus) | 27.2 | Lung ca. (small cell) | 25.0 |
| | | NCI-H69 | |
| Cerebral Cortex | 33.0 | Lung ca. (s.cell var.) | 42.3 |
| | | SHP-77 | |
| Spinal cord | 16.5 | Lung ca. (large | 25.7 |
| | | cell)NCI-H460 | |
| glio/astro U87-MG | 8.9 | Lung ca. (non-sm. | 12.0 |
| | | cell) A549 | |
| glio/astro U-118-MG | 100.0 | Lung ca. (non-s.cell) | 9.1 |
| | | NCI-H23 | |
| astrocytoma SW1783 | 14.6 | Lung ca. (non-s.cell) | 9.5 |
| | | HOP-62 | |
| neuro*; met SK-N-AS | 43.2 | Lung ca. (non-s.cl) | 10.7 |
| | | NCI-H522 | |
| astrocytoma SF-539 | 13.9 | Lung ca. (squam.) | 12.4 |
| | | SW 900 | |
| astrocytoma SNB-75 | 29.7 | Lung ca. (squam.) | 59.0 |
| | | NCI-H596 | |
| glioma SNB-19 | 13.5 | Mammary gland | 10.6 |
| glioma U251 | 43.8 | Breast ca.* (pl.ef) | 46.3 |
| | | MCF-7 | |
| glioma SF-295 | 17.7 | Breast ca.* (pl.ef) | 31.6 |
| | | MDA-MB-231 | |
| Heart (fetal) | 22.7 | Breast ca.* (pl.ef) | 15.1 |
| | | T47D | |
| Heart | 14.5 | Breast ca. BT-549 | 54.0 |
| Skeletal muscle (fetal) | 6.8 | Breast ca. MDA-N | 11.5 |
| Skeletal muscle | 55.5 | Ovary | 8.7 |
| Bone marrow | 10.7 | Ovarian ca. | 26.2 |
| | | OVCAR-3 | |
| Thymus | 5.5 | Ovarian ca. | 21.6 |
| | | OVCAR-4 | |
| Spleen | 13.3 | Ovarian ca. | 20.9 |
| | | OVCAR-5 | |
| Lymph node | 24.8 | Ovarian ca. | 12.6 |
| | | OVCAR-8 | |
| Colorectal | 8.8 | Ovarian ca. IGROV-1 | 4.4 |
| Stomach | 15.1 | Ovarian ca.* | 23.5 |
| | | (ascites) SK-OV-3 | |
| Small intestine | 28.3 | Uterus | 14.3 |
| Colon ca. SW480 | 27.5 | Placenta | 6.9 |
| Colon ca.* | 17.6 | Prostate | 9.5 |
| SW620(SW480 met) | | | |
| Colon ca. HT29 | 14.6 | Prostate ca.* (bone | 17.7 |
| | | met)PC-3 | |
| Colon ca. HCT-116 | 43.2 | Testis | 10.1 |
| Colon ca. CaCo-2 | 12.7 | Melanoma | 7.6 |
| | | Hs688(A).T | |
| Colon ca. | 22.5 | Melanoma* (met) | 6.6 |
| tissue(ODO3866) | | Hs688(B).T | |
| Colon ca. HCC-2998 | 25.2 | Melanoma UACC- | 19.6 |
| | | 62 | |
| Gastric ca.* (liver met) | 29.5 | Melanoma M14 | 39.2 |
| NCI-N87 | | | |
| Bladder | 6.1 | Melanoma LOX | 13.4 |
| | | IMVI | |
| Trachea | 13.2 | Melanoma* (met) | 21.2 |
| | | SK-MEL-5 | |
| Kidney | 15.6 | Adipose | 6.5 |

TABLE TD
Panel 4D

| | Rel. Exp. (%) | | Rel. Exp. (%) |
|---|---|---|---|
| | Ag3323, Run | | Ag3323, Run |
| Tissue Name | 165296416 | Tissue Name | 165296416 |
| Secondary Th1 act | 32.3 | HUVEC IL-1beta | 3.8 |
| Secondary Th2 act | 22.8 | HUVEC IFN gamma | 12.0 |
| Secondary Tr1 act | 29.9 | HUVEC TNF alpha + | 8.1 |
| | | IFN gamma | |
| Secondary Th1 rest | 3.8 | HUVEC TNF alpha + | 11.1 |
| | | IL4 | |
| Secondary Th2 rest | 4.3 | HUVEC IL-11 | 8.4 |
| Secondary Tr1 rest | 6.0 | Lung Microvascular EC | 7.6 |
| | | none | |
| Primary Th1 act | 33.0 | Lung Microvascular EC | 6.9 |
| | | TNF alpha + IL-1beta | |
| Primary Th2 act | 25.0 | Microvascular Dermal | 14.7 |
| | | EC none | |
| Primary Tr1 act | 40.1 | Microsvasular Dermal | 7.6 |
| | | EC TNF alpha + IL-1beta | |
| Primary Th1 rest | 17.8 | Bronchial epithelium | 17.3 |
| | | TNF alpha + IL1beta | |
| Primary Th2 rest | 11.6 | Small airway epithelium | 6.6 |
| | | none | |
| Primary Tr1 rest | 15.0 | Small airway epithelium | 18.4 |
| | | TNF alpha + IL-1beta | |
| CD45RA CD4 | 15.0 | Coronery artery SMC rest | 9.9 |
| lymphocyte act | | | |
| CD45RO CD4 | 24.7 | Coronery artery SMC | 6.5 |
| lymphocyte act | | TNF alpha + IL-1beta | |
| CD8 lymphocyte act | 19.3 | Astrocytes rest | 5.1 |
| Secondary CD8 | 22.7 | Astrocytes TNF alpha + | 3.9 |
| lymphocyte rest | | IL-1beta | |
| Secondary CD8 | 12.9 | KU-812 (Basophil) rest | 14.0 |
| lymphocyte act | | | |
| CD4 lymphocyte none | 2.9 | KU-812 (Basophil) | 22.1 |
| | | PMA/ionomycin | |
| 2ry Th1/Th2/Tr1_anti- | 5.4 | CCD1106 | 16.0 |
| CD95 CH11 | | (Keratinocytes) none | |
| LAK cells rest | 7.2 | CCD1106 | 8.1 |
| | | (Keratinocytes) | |
| | | TNF alpha + IL-1beta | |
| LAK cells IL-2 | 17.2 | Liver cirrhosis | 1.7 |
| LAK cells IL-2 + IL-12 | 15.1 | Lupus kidney | 1.0 |
| LAK cells IL-2 + IFN | 27.9 | NCI-H292 none | 30.1 |
| gamma | | | |
| LAK cells IL-2 + IL-18 | 17.7 | NCI-H292 IL-4 | 49.0 |
| LAK cells | 1.9 | NCI-H292 IL-9 | 33.2 |
| PMA/ionomycin | | | |
| NK Cells IL-2 rest | 8.4 | NCI-H292 IL-13 | 26.2 |
| Two Way MLR 3 day | 9.9 | NCI-H292 IFN gamma | 26.6 |
| Two Way MLR 5 day | 18.4 | HPAEC none | 11.7 |
| Two Way MLR 7 day | 8.9 | HPAEC TNF alpha + IL- | 7.5 |
| | | 1beta | |
| PBMC rest | 3.8 | Lung fibroblast none | 8.0 |
| PBMC PWM | 50.3 | Lung fibroblast TNF | 5.5 |
| | | alpha + IL-1beta | |
| PBMC PHA-L | 29.3 | Lung fibroblast IL-4 | 19.1 |
| Ramos (B cell) none | 33.9 | Lung fibroblast IL-9 | 15.3 |
| Ramos (B cell) | 83.5 | Lung fibroblast IL-13 | 11.4 |
| ionomycin | | | |
| B lymphocytes PWM | 100.0 | Lung fibroblast IFN | 16.5 |
| | | gamma | |
| B lymphocytes CD40L | 22.4 | Dermal fibroblast | 28.9 |
| and IL-4 | | CCD1070 rest | |
| EOL-1 dbcAMP | 10.5 | Dermal fibroblast | 31.2 |
| | | CCD1070 TNF alpha | |
| EOL-1 dbcAMP | 3.7 | Dermal fibroblast | 11.3 |
| PMA/ionomycin | | CCD1070 IL-1beta | |
| Dendritic cells none | 9.9 | Dermal fibroblast IFN | 5.2 |
| | | gamma | |
| Dendritic cells LPS | 6.3 | Dermal fibroblast IL-4 | 12.3 |
| Dendritic cells anti- | 7.3 | IBD Colitis 2 | 0.7 |
| CD40 | | | |
| Monocytes rest | 7.0 | IBD Crohn's | 1.0 |
| Monocytes LPS | 1.4 | Colon | 8.8 |
| Macrophages rest | 13.5 | Lung | 5.8 |
| Macrophages LPS | 3.5 | Thymus | 17.3 |
| HUVEC none | 11.9 | Kidney | 12.3 |
| HUVEC starved | 24.8 | | |

CNS_neurodegeneration_v1.0 Summary: Ag3323 This panel does not show differential expression of the CG57709-01 gene in Alzheimer's disease. However, this expression profile confirms the presence of this gene in the brain. Please see Panel 1.3D for discussion of utility of this gene in the central nervous system.

Panel 1.3D Summary: Ag3323—This gene is expressed at moderate levels in all samples on this panel, with highest expression in a brain cancer cell line. Expression is also seen in all the cancer cell lines on this panel. Thus, expression of this gene could be used to differentiate between this brain cancer cell line sample and other samples on this panel and as a marker for brain cancer.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at moderate to low levels in the CNS and may be a small molecule target for the treatment of neurologic diseases such as Alzheimer's disease, Parkinson's disease, epilepsy, schizophrenia, stroke and multiple sclerosis.

Panel 4D Summary: Ag3323—This gene is expressed at high to moderate levels in all samples on this panel, with highest expression in B lymphocytes stimulated with polkweed mitoger (CT=24.5). In addition, this gene is expressed at higher levels in ionomycin-activated Ramos B lymphocytes. The high levels of expression in activated B lymphocytes suggests that therapies that antagonize the function of this gene product may reduce or eliminate the symptoms in patients with autoimmune and inflammatory diseases in which B cells play a part in the initiation or progression of the disease process, such as lupus erythemratosus, Crohn's disease, ulcerative colitis, multiple sclerosis, chronic obstructive pulmonary disease, asthma, emphysema, rheumatoid arthritis, or psoriasis.

U. CG57700-01: Hydroxyacylglutathione Hydrolase (Glyoxalase II)

Expression of gene CG57700-01 was assessed using the primer-probe set Ag3311, described in Table UA. Results of the RTQ-PCR runs are shown in Table UB.

Table UA. Probe Name Ag3311

TABLE UA
Probe Name Ag3311

| | | | Start | |
|---|---|---|---|---|
| Primers | Sequences | Length | Position | SEQ ID NO: |
| Forward | 5′-acgcttagcaacctggagtt-3′ | 20 | 536 | 453 |
| Probe | TET-5′-accacgtgagagccaagctgtcct-3′-TAMRA | 24 | 582 | 454 |
| Reverse | 5′-gtcatcctcatccctcttctg-3′ | 21 | 611 | 456 |

TABLE UB
Panel 4D

| | Rel. Exp. (%) | | Rel. Exp. (%) |
|---|---|---|---|
| | Ag3311, Run | | Ag3311, Run |
| Tissue Name | 164682845 | Tissue Name | 164682845 |
| Secondary Th1 act | 10.2 | HUVEC IL-1beta | 0.0 |
| Secondary Th2 act | 3.8 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 1.6 | Microsvasular Dermal | 0.0 |
| | | EC TNF alpha + IL-1beta | |
| Primary Th1 rest | 0.0 | Bronchial epithelium | 5.1 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 4.2 |
| CD95 CH11 | | (Keratinocytes) none | |
| LAK cells rest | 0.0 | CCD1106 | 2.7 |
| | | (Keratinocytes) | |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 4.5 | NCI-H292 IFN gamma | 1.9 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 3.7 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 14.1 |
| Ramos (B cell) | 0.0 | Lung fibroblast IL-13 | 4.3 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 1.6 | Dermal fibroblast | 0.0 |
| PMA/ionomycin | | CCD1070 IL-1beta | |
| Dendritic cells none | 2.1 | Dermal fibroblast IFN | 0.0 |
| | | gamma | |
| Dendritic cells LPS | 0.0 | Dermal fibroblast IL-4 | 3.0 |
| Dendritic cells anti- | 2.5 | IBD Colitis 2 | 0.0 |
| CD40 | | | |
| Monocytes rest | 0.0 | IBD Crohn's | 0.0 |
| Monocytes LPS | 0.0 | Colon | 100.0 |
| Macrophages rest | 0.0 | Lung | 29.3 |
| Macrophages LPS | 0.0 | Thymus | 0.0 |
| HUVEC none | 0.0 | Kidney | 2.4 |
| HUVEC starved | 0.0 | | |

AI_comprehensive panel_v1.0 Summary: Ag3311—Expression of this gene is low/undetectable (CTs>35) across all of the samples on this panel (data not shown).

CNS_neurodegeneration_v1.0 Summary: Ag3311—Expression of this gene is low/undetectable (CTs>35) across all of the samples on this panel (data not shown).

General_screening_panel_v1.4 Summary: Ag3311—Expression of this gene is low/undetectable (CTs>35) across all of the samples on this panel (data not shown).

Panel 4D Summary: Ag3311—Significant expression of this gene is seen only in colon (CT=33.9). Therefore, expression of this gene can be used to distinguish between this sample and the others on the panel and between healthy and inflammed bowel. Since expression is not detectable in samples derived from Crohn's and colitis patients, therapeutic modulation of the expression or function of this gene may be useful in the treatment of inflammatory bowel disease.

V. CG58553-01: Vasolpressin Receptor

Expression of gene CG58553-01 was assessed using the primer-probe set Ag3372, described in Table VA. Results of the RTQ-PCR runs are shown in Tables VB and VC.

Table VA. Probe Name Ag3372

TABLE VA
Probe Name Ag3372

| | | | Start | SEQ ID |
|---|---|---|---|---|
| Primers | Sequences | Length | Position | NO: |
| Forward | 5′-cggatctggtcatcacaca-3′ | 19 | 1983 | 457 |
| Probe | TET-5′-ccacccacaacctcccaaggaact-3′-TAMRA | 24 | 2017 | 458 |
| Reverse | 5′-agcctcagaaggtcgagatg-3′ | 20 | 2041 | 459 |

TABLE VB
Panel 1.3D

| | Rel. Exp. (%) | | Rel. Exp. (%) |
|---|---|---|---|
| | Ag3372, | | Ag3372, |
| Tissue Name | Run 165524269 | Tissue Name | Run 165524269 |
| Liver adenocarcinoma | 0.7 | Kidney (fetal) | 0.0 |
| Pancreas | 0.2 | Renal ca. 786-0 | 0.0 |
| Pancreatic ca. CAPAN 2 | 0.0 | Renal ca. A498 | 0.1 |
| Adrenal gland | 0.0 | Renal ca. RXF 393 | 0.0 |
| Thyroid | 0.1 | Renal ca. ACHN | 0.0 |
| Salivary gland | 0.1 | Renal ca. UO-31 | 0.0 |
| Pituitary gland | 0.2 | Renal ca. TK-10 | 0.0 |
| Brain (fetal) | 0.0 | Liver | 2.1 |
| Brain (whole) | 0.3 | Liver (fetal) | 0.0 |
| Brain (amygdala) | 0.0 | Liver ca. | 0.2 |
| | | (hepatoblast) HepG2 | |
| Brain (cerebellum) | 0.1 | Lung | 2.4 |
| Brain (hippocampus) | 0.5 | Lung (fetal) | 0.2 |
| Brain (substantia nigra) | 0.2 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 0.1 |
| | | SHP-77 | |
| Spinal cord | 1.0 | Lung ca. (large | 0.0 |
| | | cell)NCI-H460 | |
| glio/astro U87-MG | 0.0 | Lung ca. (non-sm. | 0.1 |
| | | cell) A549 | |
| glio/astro U-118-MG | 0.0 | Lung ca. (non-s.cell) | 0.6 |
| | | NCI-H23 | |
| astrocytoma SW1783 | 0.0 | Lung ca. (non-s.cell) | 0.1 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.1 | Lung ca. (squam.) | 0.0 |
| | | NCI-H596 | |
| glioma SNB-19 | 0.4 | Mammary gland | 0.7 |
| glioma U251 | 0.2 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.1 |
| | | T47D | |
| Heart | 0.0 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 1.6 | Ovarian ca. | 0.2 |
| | | OVCAR-3 | |
| Thymus | 1.7 | Ovarian ca. | 0.0 |
| | | OVCAR-4 | |
| Spleen | 2.8 | Ovarian ca. | 0.2 |
| | | OVCAR-5 | |
| Lymph node | 5.5 | Ovarian ca. | 0.2 |
| | | OVCAR-8 | |
| Colorectal | 0.2 | Ovarian ca. IGROV-1 | 0.0 |
| Stomach | 1.2 | Ovarian ca.* | 0.0 |
| | | (ascites) SK-OV-3 | |
| Small intestine | 100.0 | Uterus | 0.0 |
| Colon ca. SW480 | 0.0 | Placenta | 0.8 |
| Colon ca.* | 0.0 | Prostate | 0.1 |
| SW620(SW480 met) | | | |
| Colon ca. HT29 | 0.0 | Prostate ca.* (bone | 0.0 |
| | | met)PC-3 | |
| Colon ca. HCT-116 | 0.0 | Testis | 1.4 |
| Colon ca. CaCo-2 | 0.3 | Melanoma | 0.0 |
| | | Hs688(A).T | |
| Colon ca. | 0.7 | Melanoma* (met) | 0.0 |
| tissue(ODO3866) | | Hs688(B).T | |
| Colon ca. HCC-2998 | 3.8 | Melanoma UACC- | 0.0 |
| | | 62 | |
| Gastric ca.* (liver met) | 1.0 | Melanoma M14 | 0.2 |
| NCI-N87 | | | |
| Bladder | 0.0 | Melanoma LOX | 0.2 |
| | | IMVI | |
| Trachea | 0.1 | Melanoma* (met) | 0.4 |
| | | SK-MEL-5 | |
| Kidney | 0.6 | Adipose | 1.3 |

TABLE VC
Panel 4D

| | Rel. Exp. (%) | | Rel. Exp. (%) |
|---|---|---|---|
| | Ag3372, Run | | Ag3372, Run |
| Tissue Name | 165296616 | Tissue Name | 165296616 |
| Secondary Th1 act | 1.4 | HUVEC IL-1beta | 0.0 |
| Secondary Th2 act | 1.4 | HUVEC IFN gamma | 0.0 |
| Secondary Tr1 act | 2.9 | HUVEC TNF alpha + | 0.0 |
| | | IFN gamma | |
| Secondary Th1 rest | 5.4 | HUVEC TNF alpha + | 0.0 |
| | | IL4 | |
| Secondary Th2 rest | 6.4 | HUVEC IL-11 | 0.0 |
| Secondary Tr1 rest | 12.0 | Lung Microvascular EC | 0.0 |
| | | none | |
| Primary Th1 act | 11.4 | Lung Microvascular EC | 0.0 |
| | | TNF alpha + IL-1beta | |
| Primary Th2 act | 18.9 | Microvascular Dermal | 0.0 |
| | | EC none | |
| Primary Tr1 act | 27.0 | Microsvasular Dermal | 0.0 |
| | | EC TNF alpha + IL-1beta | |
| Primary Th1 rest | 27.5 | Bronchial epithelium | 0.1 |
| | | TNF alpha + IL1beta | |
| Primary Th2 rest | 13.6 | Small airway epithelium | 0.0 |
| | | none | |
| Primary Tr1 rest | 32.8 | Small airway epithelium | 0.0 |
| | | TNF alpha + IL-1beta | |
| CD45RA CD4 | 3.0 | Coronery artery SMC rest | 0.0 |
| lymphocyte act | | | |
| CD45RO CD4 | 8.5 | Coronery artery SMC | 0.0 |
| lymphocyte act | | TNF alpha + IL-1beta | |
| CD8 lymphocyte act | 5.8 | Astrocytes rest | 0.0 |
| Secondary CD8 | 3.1 | Astrocytes TNF alpha + | 0.0 |
| lymphocyte rest | | IL-1beta | |
| Secondary CD8 | 2.9 | KU-812 (Basophil) rest | 0.0 |
| lymphocyte act | | | |
| CD4 lymphocyte none | 4.7 | KU-812 (Basophil) | 0.1 |
| | | PMA/ionomycin | |
| 2ry Th1/Th2/Tr1_anti- | 7.5 | CCD1106 | 0.0 |
| CD95 CH11 | | (Keratinocytes) none | |
| LAK cells rest | 1.8 | CCD1106 | 0.0 |
| | | (Keratinocytes) | |
| | | TNF alpha + IL-1beta | |
| LAK cells IL-2 | 5.8 | Liver cirrhosis | 1.5 |
| LAK cells IL-2 + IL-12 | 2.3 | Lupus kidney | 0.6 |
| LAK cells IL-2 + IFN | 5.5 | NCI-H292 none | 2.5 |
| gamma | | | |
| LAK cells IL-2 + IL-18 | 5.5 | NCI-H292 IL-4 | 1.8 |
| LAK cells | 2.7 | NCI-H292 IL-9 | 5.9 |
| PMA/ionomycin | | | |
| NK Cells IL-2 rest | 6.0 | NCI-H292 IL-13 | 2.3 |
| Two Way MLR 3 day | 2.1 | NCI-H292 IFN gamma | 3.0 |
| Two Way MLR 5 day | 0.9 | HPAEC none | 0.0 |
| Two Way MLR 7 day | 1.8 | HPAEC TNF alpha + IL- | 0.0 |
| | | 1beta | |
| PBMC rest | 1.5 | Lung fibroblast none | 0.0 |
| PBMC PWM | 5.6 | Lung fibroblast TNF | 0.0 |
| | | alpha + IL-1beta | |
| PBMC PHA-L | 0.9 | Lung fibroblast IL-4 | 0.0 |
| Ramos (B cell) none | 0.0 | Lung fibroblast IL-9 | 0.0 |
| Ramos (B cell) | 0.2 | Lung fibroblast IL-13 | 0.0 |
| ionomycin | | | |
| B lymphocytes PWM | 2.2 | Lung fibroblast IFN | 0.0 |
| | | gamma | |
| B lymphocytes CD40L | 3.7 | Dermal fibroblast | 0.0 |
| and IL-4 | | CCD1070 rest | |
| EOL-1 dbcAMP | 1.0 | Dermal fibroblast | 5.2 |
| | | CCD1070 TNF alpha | |
| EOL-1 dbcAMP | 0.4 | Dermal fibroblast | 0.0 |
| PMA/ionomycin | | CCD1070 IL-1beta | |
| Dendritic cells none | 0.2 | Dermal fibroblast IFN | 0.1 |
| | | gamma | |
| Dendritic cells LPS | 0.0 | Dermal fibroblast IL-4 | 0.0 |
| Dendritic cells anti- | 0.0 | IBD Colitis 2 | 0.4 |
| CD40 | | | |
| Monocytes rest | 0.3 | IBD Crohn's | 8.4 |
| Monocytes LPS | 0.2 | Colon | 100.0 |
| Macrophages rest | 0.7 | Lung | 0.9 |
| Macrophages LPS | 0.0 | Thymus | 8.1 |
| HUVEC none | 0.0 | Kidney | 6.8 |
| HUVEC starved | 0.0 | | |

Panel 1.3D Summary: Ag3372 Highest expression of the CG58553-01 gene is seen in the small intestine sample (CT=26.8). This gene encodes a novel vasopressin gene that plays a role in regulating electrolyte transport in the colon. Therefore, regulation of the transcript or the protein it encodes could be important in maintaining normal cellular homeostasis and in the treatment of Crohn's disease and ulcerative colitis.

Among tissues with metabolic function, this gene is expressed in liver and adipose. Thus, this gene product may be involved in disorders that affect these tissues, such as obesity and type II diabetes.

Low, but significant expression is also seen in the hippocampus. The hippocampus is critical for learning and memory. Thus, this gene product may have utility treating CNS disorders involving memory deficits, including Alzheimer's disease and aging.

### REFERENCES

1. Sato Y, Hanai H, Nogaki A, Hirasawa K, Kaneko E, Hayashi H, Suzuki Y. Role of the vasopressin V(1) receptor in regulating the epithelial functions of the guinea pig distal colon. Am J Physiol October 1999;277(4 Pt 1):G819-28.

Panel 4D Summary: Ag3372 In agreement with the results seen in panel 1.4, the highest level of expression of this gene is in the colon sample (CT=27.5). Interestingly, the expression is significantly lower in the IBD colitis 2 (CTh35) and IBD Crohn's (CT=30.9) samples. Therefore, alterations in the expression of this gene may be used in the treatment of Crohn's disease and ulcerative colitis.

In addition, the expression of the CG58553-01 gene in several preparations of T lymphocytes suggests that small molecule antagonists, therapeutic antibodies specific for this molecule, or the extracellular domain of this protein, may be useful to reduce or eliminate the symptoms of Crohn's disease, ulcerative colitis, multiple sclerosis, chronic obstructive pulmonary disease, asthma, emphysema, rheumatoid arthritis, lupus erythematosus, or psoriasis.

W. CG58626-01: Phospholipase

Expression of gene CG58626-01 was assessed using the primer-probe set Ag3386, described in Table WA. Results of the RTQ-PCR runs are shown in Tables WB, WC and WD.

Table WA. Probe Name Ag3386

TABLE WA
Probe Name Ag3386

| | | | Start | SEQ ID |
|---|---|---|---|---|
| Primers | Sequences | Length | Position | NO: |
| Forward | 5′-agtggcggtcaaaacttactct-3′ | 22 | 1386 | 460 |
| Probe | TET-5′-tggagacactgttgattccattactcctg-3′-TAMRA | 29 | 1411 | 461 |
| Reverse | 5′-ctgctgttcagcatatccctta-3′ | 22 | 1455 | 462 |

TABLE WB
CNS_neurodegeneration_v1.0

| | | | Rel. Exp. |
|---|---|---|---|
| | Rel. Exp. (%) | | (%) Ag3386, |
| | Ag3386, Run | | Run |
| Tissue Name | 210154893 | Tissue Name | 210154893 |
| AD 1 Hippo | 6.4 | Control (Path) 3 | 4.2 |
| | | Temporal Ctx | |
| AD 2 Hippo | 21.5 | Control (Path) 4 | 25.0 |
| | | Temporal Ctx | |
| AD 3 Hippo | 5.0 | AD 1 Occipital | 14.4 |
| | | Ctx | |
| AD 4 Hippo | 5.3 | AD 2 Occipital | 0.0 |
| | | Ctx (Missing) | |
| AD 5 hippo | 95.3 | AD 3 Occipital | 3.3 |
| | | Ctx | |
| AD 6 Hippo | 51.1 | AD 4 Occipital | 18.7 |
| | | Ctx | |
| Control 2 Hippo | 26.4 | AD 5 Occipital | 28.7 |
| | | Ctx | |
| Control 4 Hippo | 4.5 | AD 6 Occipital | 52.5 |
| | | Ctx | |
| Control (Path) 3 | 4.0 | Control 1 Occipital | 2.4 |
| Hippo | | Ctx | |
| AD 1 Temporal Ctx | 13.5 | Control 2 Occipital | 56.3 |
| | | Ctx | |
| AD 2 Temporal Ctx | 24.5 | Control 3 Occipital | 11.0 |
| | | Ctx | |
| AD 3 Temporal Ctx | 3.8 | Control 4 Occipital | 4.3 |
| | | Ctx | |
| AD 4 Temporal Ctx | 18.9 | Control (Path) 1 | 100.0 |
| | | Occipital Ctx | |
| AD 5 Inf Temporal | 95.9 | Control (Path) 2 | 8.8 |
| Ctx | | Occipital Ctx | |
| AD 5 Sup Temporal | 37.6 | Control (Path) 3 | 1.7 |
| Ctx | | Occipital Ctx | |
| AD 6 Inf Temporal | 52.5 | Control (Path) 4 | 12.3 |
| Ctx | | Occipital Ctx | |
| AD 6 Sup Temporal | 63.7 | Control 1 Parietal | 5.4 |
| Ctx | | Ctx | |
| Control 1 Temporal | 4.9 | Control 2 Parietal | 39.5 |
| Ctx | | Ctx | |
| Control 2 Temporal | 38.4 | Control 3 Parietal | 11.3 |
| Ctx | | Ctx | |
| Control 3 Temporal | 12.2 | Control (Path) 1 | 77.4 |
| Ctx | | Parietal Ctx | |
| Control 4 Temporal | 5.0 | Control (Path) 2 | 20.7 |
| Ctx | | Parietal Ctx | |
| Control (Path) 1 | 76.8 | Control (Path) 3 | 2.7 |
| Temporal Ctx | | Parietal Ctx | |
| Control (Path) 2 | 37.6 | Control (Path) 4 | 45.4 |
| Temporal Ctx | | Parietal Ctx | |

TABLE WC
General_screening_panel_v1.4

| | Rel. Exp. | | Rel. Exp. |
|---|---|---|---|
| | (%) Ag3386, | | (%) Ag3386, |
| | Run | | Run |
| Tissue Name | 217043839 | Tissue Name | 217043839 |
| Adipose | 12.2 | Renal ca. TK-10 | 10.7 |
| Melanoma* | 26.4 | Bladder | 18.4 |
| Hs688(A).T | | | |
| Melanoma* | 30.4 | Gastric ca. (liver met.) | 26.6 |
| Hs688(B).T | | NCI-N87 | |
| Melanoma* M14 | 13.1 | Gastric ca. KATO III | 0.0 |
| Melanoma* | 22.8 | Colon ca. SW-948 | 10.8 |
| LOXIMVI | | | |
| Melanoma* SK- | 22.7 | Colon ca. SW480 | 40.9 |
| MEL-5 | | | |
| Squamous cell | 11.2 | Colon ca.* (SW480 | 20.4 |
| carcinoma SCC-4 | | met) SW620 | |
| Testis Pool | 47.0 | Colon ca. HT29 | 5.2 |
| Prostate ca.* (bone | 80.1 | Colon ca. HCT-116 | 100.0 |
| met) PC-3 | | | |
| Prostate Pool | 7.1 | Colon ca. CaCo-2 | 13.8 |
| Placenta | 3.2 | Colon cancer tissue | 13.6 |
| Uterus Pool | 6.4 | Colon ca. SW1116 | 10.2 |
| Ovarian ca. | 22.8 | Colon ca. Colo-205 | 1.8 |
| OVCAR-3 | | | |
| Ovarian ca. SK- | 94.0 | Colon ca. SW-48 | 2.4 |
| OV-3 | | | |
| Ovarian ca. | 4.7 | Colon Pool | 27.7 |
| OVCAR-4 | | | |
| Ovarian ca. | 29.3 | Small Intestine Pool | 14.6 |
| OVCAR-5 | | | |
| Ovarian ca. | 12.7 | Stomach Pool | 12.2 |
| IGROV-1 | | | |
| Ovarian ca. | 11.1 | Bone Marrow Pool | 6.9 |
| OVCAR-8 | | | |
| Ovary | 11.6 | Fetal Heart | 8.1 |
| Breast ca. MCF-7 | 36.9 | Heart Pool | 6.3 |
| Breast ca. MDA- | 39.5 | Lymph Node Pool | 13.9 |
| MB-231 | | | |
| Breast ca. BT 549 | 28.5 | Fetal Skeletal Muscle | 3.6 |
| Breast ca. T47D | 52.9 | Skeletal Muscle Pool | 6.7 |
| Breast ca. MDA-N | 11.3 | Spleen Pool | 17.1 |
| Breast Pool | 28.1 | Thymus Pool | 26.1 |
| Trachea | 11.0 | CNS cancer | 33.2 |
| | | (glio/astro) U87-MG | |
| Lung | 6.0 | CNS cancer | 44.1 |
| | | (glio/astro) U-118-MG | |
| Fetal Lung | 39.2 | CNS cancer | 44.4 |
| | | (neuro; met) SK-N-AS | |
| Lung ca. NCI-N417 | 6.3 | CNS cancer (astro) | 10.4 |
| | | SF-539 | |
| Lung ca. LX-1 | 33.9 | CNS cancer (astro) | 27.7 |
| | | SNB-75 | |
| Lung ca. NCI-H146 | 14.3 | CNS cancer (glio) | 10.2 |
| | | SNB-19 | |
| Lung ca. SHP-77 | 73.2 | CNS cancer (glio) SF- | 28.7 |
| | | 295 | |
| Lung ca. A549 | 25.3 | Brain (Amygdala) | 23.2 |
| | | Pool | |
| Lung ca. NCI-H526 | 5.8 | Brain (cerebellum) | 19.8 |
| Lung ca. NCI-H23 | 30.1 | Brain (fetal) | 35.6 |
| Lung ca. NCI-H460 | 20.2 | Brain (Hippocampus) | 25.2 |
| | | Pool | |
| Lung ca. HOP-62 | 11.9 | Cerebral Cortex Pool | 39.2 |
| Lung ca. NCI-H522 | 20.7 | Brain (Substantia | 23.0 |
| | | nigra) Pool | |
| Liver | 0.7 | Brain (Thalamus) Pool | 45.7 |
| Fetal Liver | 29.5 | Brain (whole) | 24.0 |
| Liver ca. HepG2 | 10.1 | Spinal Cord Pool | 22.5 |
| Kidney Pool | 21.3 | Adrenal Gland | 8.5 |
| Fetal Kidney | 19.5 | Pituitary gland Pool | 7.0 |
| Renal ca. 786-0 | 15.9 | Salivary Gland | 1.9 |
| Renal ca. A498 | 3.5 | Thyroid (female) | 3.2 |
| Renal ca. ACHN | 8.0 | Pancreatic ca. | 3.7 |
| | | CAPAN2 | |
| Renal ca. UO-31 | 12.2 | Pancreas Pool | 18.2 |

TABLE WD
Panel 4D

| | Rel. Exp. (%) | | Rel. Exp. (%) |
|---|---|---|---|
| | Ag3386, Run | | Ag3386, Run |
| Tissue Name | 165296474 | Tissue Name | 165296474 |
| Secondary Th1 act | 30.4 | HUVEC IL-1beta | 2.0 |
| Secondary Th2 act | 35.6 | HUVEC IFN gamma | 3.3 |
| Secondary Tr1 act | 27.9 | HUVEC TNF alpha + | 3.8 |
| | | IFN gamma | |
| Secondary Th1 rest | 8.9 | HUVEC TNF alpha + | 3.3 |
| | | IL4 | |
| Secondary Th2 rest | 8.0 | HUVEC IL-11 | 1.5 |
| Secondary Tr1 rest | 11.3 | Lung Microvascular EC | 5.5 |
| | | none | |
| Primary Th1 act | 57.4 | Lung Microvascular EC | 4.8 |
| | | TNF alpha + IL-1beta | |
| Primary Th2 act | 36.9 | Microvascular Dermal | 3.7 |
| | | EC none | |
| Primary Tr1 act | 51.1 | Microsvasular Dermal | 3.3 |
| | | EC TNF alpha + IL-1beta | |
| Primary Th1 rest | 54.0 | Bronchial epithelium | 5.5 |
| | | TNF alpha + IL1beta | |
| Primary Th2 rest | 18.8 | Small airway epithelium | 2.1 |
| | | none | |
| Primary Tr1 rest | 24.7 | Small airway epithelium | 6.1 |
| | | TNF alpha + IL-1beta | |
| CD45RA CD4 | 12.4 | Coronery artery SMC rest | 4.6 |
| lymphocyte act | | | |
| CD45RO CD4 | 33.9 | Coronery artery SMC | 2.4 |
| lymphocyte act | | TNF alpha + IL-1beta | |
| CD8 lymphocyte act | 29.3 | Astrocytes rest | 3.0 |
| Secondary CD8 | 26.1 | Astrocytes TNF alpha + | 3.0 |
| lymphocyte rest | | IL-1beta | |
| Secondary CD8 | 20.7 | KU-812 (Basophil) rest | 12.1 |
| lymphocyte act | | | |
| CD4 lymphocyte none | 1.1 | KU-812 (Basophil) | 27.7 |
| | | PMA/ionomycin | |
| 2ry Th1/Th2/Tr1_anti- | 12.8 | CCD1106 | 2.7 |
| CD95 CH11 | | (Keratinocytes) none | |
| LAK cells rest | 12.0 | CCD1106 | 0.8 |
| | | (Keratinocytes) | |
| | | TNF alpha + IL-1beta | |
| LAK cells IL-2 | 24.3 | Liver cirrhosis | 0.3 |
| LAK cells IL-2 + IL-12 | 28.7 | Lupus kidney | 0.7 |
| LAK cells IL-2 + IFN | 42.0 | NCI-H292 none | 8.1 |
| gamma | | | |
| LAK cells IL-2 + IL-18 | 45.1 | NCI-H292 IL-4 | 9.5 |
| LAK cells | 8.8 | NCI-H292 IL-9 | 8.5 |
| PMA/ionomycin | | | |
| NK Cells IL-2 rest | 21.8 | NCI-H292 IL-13 | 4.5 |
| Two Way MLR 3 day | 18.7 | NCI-H292 IFN gamma | 3.6 |
| Two Way MLR 5 day | 11.0 | HPAEC none | 2.5 |
| Two Way MLR 7 day | 10.9 | HPAEC TNF alpha + IL- | 3.0 |
| | | 1beta | |
| PBMC rest | 4.5 | Lung fibroblast none | 4.6 |
| PBMC PWM | 66.0 | Lung fibroblast TNF | 3.3 |
| | | alpha + IL-1beta | |
| PBMC PHA-L | 17.9 | Lung fibroblast IL-4 | 12.1 |
| Ramos (B cell) none | 26.1 | Lung fibroblast IL-9 | 12.3 |
| Ramos (B cell) | 100.0 | Lung fibroblast IL-13 | 6.7 |
| ionomycin | | | |
| B lymphocytes PWM | 88.9 | Lung fibroblast IFN | 16.6 |
| | | gamma | |
| B lymphocytes CD40L | 49.3 | Dermal fibroblast | 8.2 |
| and IL-4 | | CCD1070 rest | |
| EOL-1 dbcAMP | 13.0 | Dermal fibroblast | 37.1 |
| | | CCD1070 TNF alpha | |
| EOL-1 dbcAMP | 9.5 | Dermal fibroblast | 4.4 |
| PMA/ionomycin | | CCD1070 IL-1beta | |
| Dendritic cells none | 8.9 | Dermal fibroblast IFN | 6.0 |
| | | gamma | |
| Dendritic cells LPS | 5.4 | Dermal fibroblast IL-4 | 12.1 |
| Dendritic cells anti- | 4.3 | IBD Colitis 2 | 0.7 |
| CD40 | | | |
| Monocytes rest | 4.7 | IBD Crohn's | 0.5 |
| Monocytes LPS | 2.6 | Colon | 3.4 |
| Macrophages rest | 8.8 | Lung | 4.9 |
| Macrophages LPS | 2.8 | Thymus | 4.1 |
| HUVEC none | 2.8 | Kidney | 13.0 |
| HUVEC starved | 6.4 | | |

CNS_neurodegeneration_v1.0 Summary: Ag3386 This panel confirms the expression of this gene at moderate to low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3386 This gene is moderately expressed in most of the samples on this panel. Based on expression in this panel, this gene may be involved in gastric, pancreatic, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

This gene product is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

In addition, this gene is expressed at moderate levels in the CNS. Therefore, this gene may play a role in central nervous system disorders such as Alzheimer's disease, Parkinson's disease, epilepsy, multiple sclerosis, schizophrenia and depression.

Panel 4D Summary: Ag3386 The CG58626-01 transcript is expressed ubiquitously in this panel. Highest expression of this transcript is seen in activated Ramos cells and activated B cells (CTs=27). The expression of this transcript in activated lymphoid cells when compared to non activated cells suggests that the CG58626-01 gene may be important for the diagnosis or pathogenesis of immune mediated diseases. Therefore, modulation of the expression and/or activity of this gene product might important for the treatment of autoimmune diseases, allergy, and delayed type hypersensitivity.

X. CG57597-01: Hypothetical Protein

Expression of gene CG57597-01 was assessed using the primer-probe set Ag3293, described in Table XA.

Table XA. Probe Name Ag3293

TABLE XA
Probe Name Ag3293

| | | | Start | SEQ ID |
|---|---|---|---|---|
| Primers | Sequences | Length | Position | NO: |
| Forward | 5′-cagaaacctgtgaactctgcat-3′ | 22 | 40 | 463 |
| Probe | TET-5′-atgcaccaccactcctggctaatttt-3′-TAMRA | 26 | 69 | 464 |
| Reverse | 5′-ataaaaggtttgagccggatt-3′ | 21 | 115 | 465 |

CNS_neurodegeneration_v1.0 Summary: Ag3293—Expression of this gene is low/undetectable (CTs>35) across all of the samples on this panel (data not shown).

General_screening_panel_v1.4 Summary: Ag3293—Expression of this gene is low/undetectable (CTs>35) across all of the samples on this panel (data not shown).

Panel 4D Summary: Ag3293—Expression of this gene is low/undetectable (CTs>35) across all of the samples on this panel (data not shown).

Y. CG57804-01: talin

Expression of gene CG57804-01 was assessed using the primer-probe set Ag3337, described in Table YA. Results of the RTQ-PCR runs are shown in Tables YB, YC and YD.

Table YA. Probe Name Ag3337

TABLE YA
Probe Name Ag3337

| | | | Start | SEQ ID |
|---|---|---|---|---|
| Primers | Sequences | Length | Position | NO: |
| Forward | 5′-ggatttcaagcccagatacaat-3′ | 22 | 781 | 466 |
| Probe | TET-5′-tggacctcatgtggaacataaacaca-3′-TAMRA | 26 | 804 | 467 |
| Reverse | 5′-ggcaggaattccttcagatc-3′ | 20 | 844 | 468 |

TABLE YB
CNS_neurodegeneration_v1.0

| | | | Rel. Exp. |
|---|---|---|---|
| | Rel. Exp. (%) | | (%) Ag3337, |
| | Ag3337, Run | | Run |
| Tissue Name | 210138775 | Tissue Name | 210138775 |
| AD 1 Hippo | 6.8 | Control (Path) 3 | 3.6 |
| | | Temporal Ctx | |
| AD 2 Hippo | 25.3 | Control (Path) 4 | 22.4 |
| | | Temporal Ctx | |
| AD 3 Hippo | 3.6 | AD 1 Occipital Ctx | 5.6 |
| AD 4 Hippo | 5.7 | AD 2 Occipital Ctx | 0.0 |
| | | (Missing) | |
| AD 5 Hippo | 78.5 | AD 3 Occipital Ctx | 2.2 |
| AD 6 Hippo | 27.5 | AD 4 Occipital Ctx | 14.7 |
| Control 2 Hippo | 27.4 | AD 5 Occipital Ctx | 44.1 |
| Control 4 Hippo | 8.1 | AD 6 Occipital Ctx | 16.6 |
| Control (Path) 3 | 4.3 | Control 1 Occipital | 1.6 |
| Hippo | | Ctx | |
| AD 1 Temporal | 7.6 | Control 2 Occipital | 67.8 |
| Ctx | | Ctx | |
| AD 2 Temporal | 24.5 | Control 3 Occipital | 11.9 |
| Ctx | | Ctx | |
| AD 3 Temporal | 3.3 | Control 4 Occipital | 3.0 |
| Ctx | | Ctx | |
| AD 4 Temporal | 15.3 | Control (Path) 1 | 89.5 |
| Ctx | | Occipital Ctx | |
| AD 5 Inf Temporal | 89.5 | Control (Path) 2 | 8.2 |
| Ctx | | Occipital Ctx | |
| AD 5 Sup Temporal | 35.8 | Control (Path) 3 | 0.6 |
| Ctx | | Occipital Ctx | |
| AD 6 Inf Temporal | 27.4 | Control (Path) 4 | 10.3 |
| Ctx | | Occipital Ctx | |
| AD 6 Sup | 32.8 | Control 1 Parietal | 3.6 |
| Temporal Ctx | | Ctx | |
| Control 1 | 3.4 | Control 2 Parietal | 23.7 |
| Temporal Ctx | | Ctx | |
| Control 2 | 47.6 | Control 3 Parietal | 14.1 |
| Temporal Ctx | | Ctx | |
| Control 3 | 12.4 | Control (Path) 1 | 100.0 |
| Temporal Ctx | | Parietal Ctx | |
| Control 3 | 5.8 | Control (Path) 2 | 21.9 |
| Temporal Ctx | | Parietal Ctx | |
| Control (Path) 1 | 64.2 | Control (Path) 3 | 2.0 |
| Temporal Ctx | | Parietal Ctx | |
| Control (Path) 2 | 42.0 | Control (Path) 4 | 39.2 |
| Temporal Ctx | | Parietal Ctx | |

TABLE YC
General_screening_panel_v1.4

| | Rel. Exp. | | Rel. Exp. |
|---|---|---|---|
| | (%) Ag3337, | | (%) Ag3337, |
| | Run | | Run |
| Tissue Name | 215773748 | Tissue Name | 215773748 |
| Adipose | 20.2 | Renal ca. TK-10 | 22.1 |
| Melanoma* | 58.6 | Bladder | 14.2 |
| Hs688(A).T | | | |
| Melanoma* | 22.8 | Gastric ca. (liver met.) | 16.2 |
| Hs688(B).T | | NCI-N87 | |
| Melanoma* M14 | 5.7 | Gastric ca. KATO | 100.0 |
| | | III | |
| Melanoma* | 5.5 | Colon ca. SW- | 16.3 |
| LOXIMVI | | 948 | |
| Melanoma* SK- | 3.4 | Colon ca. SW480 | 4.2 |
| MEL-5 | | | |
| Squamous cell | 4.4 | Colon ca.* (SW480 | 2.6 |
| carcinoma SCC-4 | | met) SW620 | |
| Testis Pool | 5.1 | Colon ca. HT29 | 0.7 |
| Prostate ca.* (bone | 6.4 | Colon ca. HCT-116 | 7.6 |
| met) PC-3 | | | |
| Prostate Pool | 3.4 | Colon ca. CaCo-2 | 81.8 |
| Placenta | 1.6 | Colon cancer tissue | 1.7 |
| Uterus Pool | 2.1 | Colon ca. SW1116 | 1.6 |
| Ovarian ca. | 8.9 | Colon ca. Colo-205 | 0.1 |
| OVCAR-3 | | | |
| Ovarian ca. SK- | 32.1 | Colon ca. SW-48 | 3.2 |
| OV-3 | | | |
| Ovarian ca. | 7.2 | Colon Pool | 8.0 |
| OVCAR-4 | | | |
| Ovarian ca. | 21.0 | Small Intestine Pool | 7.9 |
| OVCAR-5 | | | |
| Ovarian ca. | 23.5 | Stomach Pool | 5.7 |
| IGROV-1 | | | |
| Ovarian ca. | 5.4 | Bone Marrow Pool | 3.8 |
| OVCAR-8 | | | |
| Ovary | 11.7 | Fetal Heart | 24.8 |
| Breast ca. MCF-7 | 5.1 | Heart Pool | 10.2 |
| Breast ca. MDA- | 19.5 | Lymph Node Pool | 10.6 |
| MB-231 | | | |
| Breast ca. BT 549 | 11.7 | Fetal Skeletal Muscle | 30.1 |
| Breast ca. T47D | 30.8 | Skeletal Muscle Pool | 24.8 |
| Breast ca. MDA-N | 5.0 | Spleen Pool | 2.9 |
| Breast Pool | 6.9 | Thymus Pool | 0.0 |
| Trachea | 10.3 | CNS cancer | 10.7 |
| | | (glio/astro) U87-MG | |
| Lung | 2.2 | CNS cancer | 68.3 |
| | | (glio/astro) U-118-MG | |
| Fetal Lung | 10.8 | CNS cancer | 23.5 |
| | | (neuro; met) SK-N-AS | |
| Lung ca. NCI-N417 | 0.8 | CNS cancer (astro) | 21.5 |
| | | SF-539 | |
| Lung ca. LX-1 | 1.7 | CNS cancer (astro) | 40.3 |
| | | SNB-75 | |
| Lung ca. NCI-H146 | 0.4 | CNS cancer (glio) | 27.7 |
| | | SNB-19 | |
| Lung ca. SHP-77 | 11.9 | CNS cancer (glio) SF- | 38.2 |
| | | 295 | |
| Lung ca. A549 | 13.6 | Brain (Amygdala) | 28.7 |
| | | Pool | |
| Lung ca. NCI-H526 | 7.6 | Brain (cerebellum) | 38.7 |
| Lung ca. NCI-H23 | 10.2 | Brain (fetal) | 58.6 |
| Lung ca. NCI-H460 | 5.1 | Brain (Hippocampus) | 25.7 |
| | | Pool | |
| Lung ca. HOP-62 | 3.8 | Cerebral Cortex Pool | 59.0 |
| Lung ca. NCI-H522 | 10.1 | Brain (Substantia | 39.2 |
| | | nigra) Pool | |
| Liver | 0.3 | Brain (Thalamus) Pool | 51.4 |
| Fetal Liver | 15.3 | Brain (whole) | 58.2 |
| Liver ca. HepG2 | 53.2 | Spinal Cord Pool | 18.6 |
| Kidney Pool | 18.4 | Adrenal Gland | 11.1 |
| Fetal Kidney | 11.4 | Pituitary gland Pool | 4.7 |
| Renal ca. 786-0 | 31.0 | Salivary Gland | 14.0 |
| Renal ca. A498 | 0.7 | Thyroid (female) | 4.9 |
| Renal ca. ACHN | 20.3 | Pancreatic ca. | 7.5 |
| | | CAPAN2 | |
| Renal ca. UO-31 | 8.1 | Pancreas Pool | 9.4 |

TABLE YD
Panel 4D

| | Rel. Exp. (%) | | Rel. Exp. (%) |
|---|---|---|---|
| | Ag3337, Run | | Ag3337, Run |
| Tissue Name | 165725932 | Tissue Name | 165725932 |
| Secondary Th1 act | 0.0 | HUVEC IL-1beta | 0.7 |
| Secondary Th2 act | 0.0 | HUVEC IFN gamma | 3.9 |
| Secondary Tr1 act | 0.4 | HUVEC TNF alpha + | 0.3 |
| | | IFN gamma | |
| Secondary Th1 rest | 0.4 | HUVEC TNF alpha + | 0.6 |
| | | IL4 | |
| Secondary Th2 rest | 0.0 | HUVEC IL-11 | 0.3 |
| Secondary Tr1 rest | 0.3 | Lung Microvascular EC | 2.1 |
| | | none | |
| Primary Th1 act | 0.3 | Lung Microvascular EC | 5.1 |
| | | TNF alpha + IL-1beta | |
| Primary Th2 act | 1.3 | Microvascular Dermal | 16.4 |
| | | EC none | |
| Primary Tr1 act | 0.6 | Microsvasular Dermal | 9.8 |
| | | EC TNF alpha + IL-1beta | |
| Primary Th1 rest | 1.3 | Bronchial epithelium | 1.2 |
| | | TNF alpha + IL1beta | |
| Primary Th2 rest | 0.6 | Small airway epithelium | 1.3 |
| | | none | |
| Primary Tr1 rest | 0.3 | Small airway epithelium | 2.1 |
| | | TNF alpha + IL-1beta | |
| CD45RA CD4 | 18.7 | Coronery artery SMC rest | 9.9 |
| lymphocyte act | | | |
| CD45RO CD4 | 0.6 | Coronery artery SMC | 3.5 |
| lymphocyte act | | TNF alpha + IL-1beta | |
| CD8 lymphocyte act | 1.2 | Astrocytes rest | 100.0 |
| Secondary CD8 | 0.9 | Astrocytes TNF alpha + | 65.5 |
| lymphocyte rest | | IL-1beta | |
| Secondary CD8 | 0.2 | KU-812 (Basophil) rest | 11.7 |
| lymphocyte act | | | |
| CD4 lymphocyte none | 0.0 | KU-812 (Basophil) | 8.5 |
| | | PMA/ionomycin | |
| 2ry Th1/Th2/Tr1_anti- | 0.3 | CCD1106 | 2.0 |
| CD95 CH11 | | (Keratinocytes) none | |
| LAK cells rest | 4.0 | CCD1106 | 2.0 |
| | | (Keratinocytes) | |
| | | TNF alpha + IL-1beta | |
| LAK cells IL-2 | 1.2 | Liver cirrhosis | 3.6 |
| LAK cells IL-2 + IL-12 | 0.4 | Lupus Kidney | 13.6 |
| LAK cells IL-2 + IFN | 2.1 | NCI-H292 none | 11.0 |
| gamma | | | |
| LAK cells IL-2 + IL-18 | 1.2 | NCI-H292 IL-4 | 25.0 |
| LAK cells | 2.0 | NCI-H292 IL-9 | 15.6 |
| PMA/ionomycin | | | |
| NK Cells IL-2 rest | 0.0 | NCI-H292 IL-13 | 12.5 |
| Two Way MLR 3 day | 5.2 | NCI-H292 IFN gamma | 4.6 |
| Two Way MLR 5 day | 2.7 | HPAEC none | 1.5 |
| Two Way MLR 7 day | 1.8 | HPAEC TNF alpha + IL- | 2.5 |
| | | 1beta | |
| PBMC rest | 0.2 | Lung fibroblast none | 80.1 |
| PBMC PWM | 1.9 | Lung fibroblast TNF | 22.7 |
| | | alpha + IL-1beta | |
| PBMC PHA-L | 0.3 | Lung fibroblast IL-4 | 97.3 |
| Ramos (B cell) none | 2.4 | Lung fibroblast IL-9 | 47.6 |
| Ramos (B cell) | 2.1 | Lung fibroblast IL-13 | 81.8 |
| ionomycin | | | |
| B lymphocytes PWM | 0.7 | Lung fibroblast IFN | 50.7 |
| | | gamma | |
| B lymphocytes CD40L | 0.6 | Dermal fibroblast | 94.6 |
| and IL-4 | | CCD1070 rest | |
| EOL-1 dbcAMP | 4.9 | Dermal fibroblast | 43.2 |
| | | CCD1070 TNF alpha | |
| EOL-1 dbcAMP | 1.2 | Dermal fibroblast | 31.2 |
| PMA/ionomycin | | CCD1070 IL-1beta | |
| Dendritic cells none | 12.8 | Dermal fibroblast IFN | 14.2 |
| | | gamma | |
| Dendritic cells LPS | 1.3 | Dermal fibroblast IL-4 | 95.9 |
| Dendritic cells anti- | 11.4 | IBD Colitis 2 | 1.2 |
| CD40 | | | |
| Monocytes rest | 0.5 | IBD Crohn's | 9.1 |
| Monocytes LPS | 1.3 | Colon | 60.7 |
| Macrophages rest | 13.6 | Lung | 8.0 |
| Macrophages LPS | 2.8 | Thymus | 39.2 |
| HUVEC none | 1.0 | Kidney | 10.4 |
| HUVEC starved | 1.6 | | |

CNS_neurodegeneration_v1.0 Summary: Ag3337—This panel confirms the expression of this gene at low to moderate levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders

General_screening_panel_v1.4 Summary: Ag3337—This gene is expressed in almost all samples on this panel. This gene is expressed at moderate levels in the CNS. Therefore, this gene may play a role in central nervous system disorders such as Alzheimer's disease, Parkinson's disease, epilepsy, multiple sclerosis, schizophrenia and depression.

In addition, this gene is also expressed in adipose, pancreas, adrenal, thyroid, pituitary, skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

Panel 4D Summary: Ag3337 This gene is most highly expressed in resting astrocytes (CT=28.9). In addition, this gene is highly expressed in a cluster of treated and untreated samples derived from lung and dermal fibroblasts. Thus, therapeutic modulation of the expression or function of this gene may be effective in the treatment of pathological and inflammatory lung and skin diseases, such as psoriasis, asthma, emphysema, and allergies.

Z. CG57551-01: NAC-1 Like Gene

Expression of gene CG57551-01 was assessed using the primer-probe set Ag3282, described in Table ZA. Results of the RTQ-PCR runs are shown in Tables ZB, ZC and ZD.

Table ZA. Probe Name Ag3282

TABLE ZA
Probe Name Ag3282

| | | | Start | SEQ ID |
|---|---|---|---|---|
| Primers | Sequences | Length | Position | NO: |
| Forward | 5′-cagatcctcagcttctgctaca-3′ | 22 | 269 | 469 |
| Probe | TET-5′-accagttcctgctcatgtacacggct-3′-TAMRA | 26 | 318 | 470 |
| Reverse | 5′-atctcctggatctgcaggaa-3′ | 20 | 347 | 471 |

TABLE ZB
CNS_neurodegeneration_v1.0

| | Rel. Exp. (%) Ag3282, | | Rel. Exp. (%) Ag3282, |
|---|---|---|---|
| Tissue Name | Run 210060482 | Tissue Name | Run 210060482 |
| AD 1 Hippo | 22.8 | Control (Path) 3 | 9.7 |
| | | Temporal Ctx | |
| AD 2 Hippo | 49.0 | Control (Path) 4 | 24.3 |
| | | Temporal Ctx | |
| AD 3 Hippo | 11.5 | AD 1 Occipital | 16.5 |
| | | Ctx | |
| AD 4 Hippo | 12.3 | AD 2 Occipital | 0.0 |
| | | Ctx (Missing) | |
| AD 5 hippo | 66.9 | AD 3 Occipital | 10.2 |
| | | Ctx | |
| AD 6 Hippo | 59.9 | AD 4 Occipital | 18.2 |
| | | Ctx | |
| Control 2 Hippo | 49.3 | AD 5 Occipital | 9.5 |
| | | Ctx | |
| Control 4 Hippo | 18.7 | AD 6 Occipital | 41.5 |
| | | Ctx | |
| Control (Path) 3 | 6.3 | Control 1 Occipital | 6.8 |
| Hippo | | Ctx | |
| AD 1 Temporal Ctx | 19.2 | Control 2 Occipital | 91.4 |
| | | Ctx | |
| AD 2 Temporal Ctx | 40.3 | Control 3 Occipital | 16.3 |
| | | Ctx | |
| AD 3 Temporal Ctx | 14.3 | Control 4 Occipital | 12.2 |
| | | Ctx | |
| AD 4 Temporal Ctx | 18.3 | Control (Path) 1 | 100.0 |
| | | Occipital Ctx | |
| AD 5 Inf Temporal | 66.0 | Control (Path) 2 | 9.2 |
| Ctx | | Occipital Ctx | |
| AD 5 SupTemporal | 37.4 | Control (Path) 3 | 5.3 |
| Ctx | | Occipital Ctx | |
| AD 6 Inf Temporal | 36.1 | Control (Path) 4 | 15.8 |
| Ctx | | Occipital Ctx | |
| AD 6 Sup Temporal | 34.4 | Control 1 Parietal | 11.7 |
| Ctx | | Ctx | |
| Control 1 Temporal | 10.0 | Control 2 Parietal | 34.2 |
| Ctx | | Ctx | |
| Control 2 Temporal | 74.7 | Control 3 Parietal | 23.3 |
| Ctx | | Ctx | |
| Control 3 Temporal | 15.0 | Control (Path) 1 | 72.7 |
| Ctx | | Parietal Ctx | |
| Control 4 Temporal | 15.5 | Control (Path) 2 | 21.6 |
| Ctx | | Parietal Ctx | |
| Control (Path) 1 | 74.2 | Control (Path) 3 | 5.5 |
| Temporal Ctx | | Parietal Ctx | |
| Control (Path) 2 | 31.2 | Control (Path) 4 | 35.8 |
| Temporal Ctx | | Parietal Ctx | |

TABLE ZC
General_screening_panel_v1.4

| | Rel. Exp. (%) Ag3282, Run | | Rel. Exp. (%) Ag3282, Run |
|---|---|---|---|
| Tissue Name | 216512995 | Tissue Name | 216512995 |
| Adipose | 1.8 | Renal ca. TK-10 | 22.7 |
| Melanoma* | 16.3 | Bladder | 6.3 |
| Hs688(A).T | | | |
| Melanoma* | 25.0 | Gastric ca. (liver met.) | 47.0 |
| Hs688(B).T | | NCI-N87 | |
| Melanoma* M14 | 25.3 | Gastric ca. KATO III | 45.7 |
| Melanoma* | 21.6 | Colon ca. SW-948 | 19.3 |
| LOXIMVI | | | |
| Melanoma* SK- | 17.0 | Colon ca. SW480 | 50.3 |
| MEL-5 | | | |
| Squamous cell | 24.7 | Colon ca.* (SW480 | 25.9 |
| carcinoma SCC-4 | | met) SW620 | |
| Testis Pool | 6.1 | Colon ca. HT29 | 17.7 |
| Prostate ca.* (bone | 67.8 | Colon ca. HCT-116 | 100.0 |
| met) PC-3 | | | |
| Prostate Pool | 3.5 | Colon ca. CaCo-2 | 29.1 |
| Placenta | 9.6 | Colon cancer tissue | 14.0 |
| Uterus Pool | 0.6 | Colon ca. SW1116 | 12.7 |
| Ovarian ca. | 41.2 | Colon ca. Colo-205 | 7.6 |
| OVCAR-3 | | | |
| Ovarian ca. SK- | 65.5 | Colon ca. SW-48 | 5.8 |
| OV-3 | | | |
| Ovarian ca. | 35.8 | Colon Pool | 4.9 |
| OVCAR-4 | | | |
| Ovarian ca. | 37.6 | Small Intestine Pool | 2.4 |
| OVCAR-5 | | | |
| Ovarian ca. | 28.9 | Stomach Pool | 3.3 |
| IGROV-1 | | | |
| Ovarian ca. | 14.2 | Bone Marrow Pool | 1.5 |
| OVCAR-8 | | | |
| Ovary | 3.9 | Fetal Heart | 3.0 |
| Breast ca. MCF-7 | 42.3 | Heart Pool | 2.2 |
| Breast ca. MDA- | 69.7 | Lymph Node Pool | 5.6 |
| MB-231 | | | |
| Breast ca. BT 549 | 51.4 | Fetal Skeletal Muscle | 1.5 |
| Breast ca. T47D | 86.5 | Skeletal Muscle Pool | 0.0 |
| Breast ca. MDA-N | 26.4 | Spleen Pool | 2.8 |
| Breast Pool | 4.6 | Thymus Pool | 3.8 |
| Trachea | 8.7 | CNS cancer | 60.3 |
| | | (glio/astro) U87-MG | |
| Lung | 0.2 | CNS cancer | 100.0 |
| | | (glio/astro) U-118-MG | |
| Fetal Lung | 6.3 | CNS cancer | 47.3 |
| | | (neuro; met) SK-N-AS | |
| Lung ca. NCI-N417 | 8.4 | CNS cancer (astro) SF- | 22.8 |
| | | 539 | |
| Lung ca. LX-1 | 17.3 | CNS cancer (astro) | 47.3 |
| | | SNB-75 | |
| Lung ca. NCI-H146 | 15.3 | CNS cancer (glio) | 29.3 |
| | | SNB-19 | |
| Lung ca. SHP-77 | 16.5 | CNS cancer (glio) SF- | 49.3 |
| | | 295 | |
| Lung ca. A549 | 27.2 | Brain (Amygdala) Pool | 6.9 |
| Lung ca. NCI-H526 | 6.1 | Brain (cerebellum) | 15.1 |
| Lung ca. NCI-H23 | 25.9 | Brain (fetal) | 9.2 |
| Lung ca. NCI-H460 | 8.0 | Brain (Hippocampus) | 8.9 |
| | | Pool | |
| Lung ca. HOP-62 | 11.9 | Cerebral Cortex Pool | 13.4 |
| Lung ca. NCI-H522 | 21.9 | Brain (Substantia | 15.3 |
| | | nigra) Pool | |
| Liver | 1.7 | Brain (Thalamus) Pool | 11.5 |
| Fetal Liver | 9.8 | Brain (whole) | 12.6 |
| Liver ca. HepG2 | 18.2 | Spinal Cord Pool | 7.1 |
| Kidney Pool | 5.0 | Adrenal Gland | 5.3 |
| Fetal Kidney | 3.4 | Pituitary gland Pool | 1.9 |
| Renal ca. 786-0 | 42.0 | Salivary Gland | 4.0 |
| Renal ca. A498 | 16.7 | Thyroid (female) | 3.6 |
| Renal ca. ACHN | 13.9 | Pancreatic ca. | 15.5 |
| | | CAPAN2 | |
| Real ca. UO-31 | 17.4 | Pancreas Pool | 6.6 |

TABLE ZD
Panel 4D

| | Rel. Exp. (%) | | Rel. Exp. (%) |
|---|---|---|---|
| | Ag3282, Run | | Ag3282, Run |
| Tissue Name | 164634321 | Tissue Name | 164634321 |
| Secondary Th1 act | 52.9 | HUVEC IL-1beta | 13.6 |
| Secondary Th2 act | 67.8 | HUVEC IFN gamma | 42.9 |
| Secondary Tr1 act | 75.3 | HUVEC TNF alpha + | 37.1 |
| | | IFN gamma | |
| Secondary Th1 rest | 8.4 | HUVEC TNF alpha + | 42.6 |
| | | IL4 | |
| Secondary Th2 rest | 11.4 | HUVEC IL-11 | 25.9 |
| Secondary Tr1 rest | 12.2 | Lung Microvascular EC | 41.2 |
| | | none | |
| Primary Th1 act | 53.6 | Lung Microvascular EC | 36.3 |
| | | TNF alpha + IL-1beta | |
| Primary Th2 act | 44.4 | Microvascular Dermal | 50.3 |
| | | EC none | |
| Primary Tr1 act | 60.7 | Microsvasular Dermal | 33.0 |
| | | EC TNF alpha + IL-1beta | |
| Primary Th1 rest | 37.6 | Bronchial epithelium | 51.4 |
| | | TNF alpha + IL1beta | |
| Primary Th2 rest | 15.8 | Small airway epithelium | 23.3 |
| | | none | |
| Primary Tr1 rest | 18.3 | Small airway epithelium | 71.7 |
| | | TNF alpha + IL-1beta | |
| CD45RA CD4 | 33.0 | Coronery artery SMC rest | 43.5 |
| lymphocyte act | | | |
| CD45RO CD4 | 54.7 | Coronery artery SMC | 31.0 |
| lymphocyte act | | TNF alpha + IL-1beta | |
| CD8 lymphocyte act | 42.9 | Astrocytes rest | 38.4 |
| Secondary CD8 | 50.3 | Astrocytes TNF alpha + | 37.1 |
| lymphocyte rest | | IL-1beta | |
| Secondary CD8 | 32.5 | KU-812 (Basophil) rest | 36.1 |
| lymphocyte act | | | |
| CD4 lymphocyte none | 2.4 | KU-812 (Basophil) | 90.8 |
| | | PMA/ionomycin | |
| 2ry Th1/Th2/Tr1_anti- | 11.7 | CCD1106 | 64.2 |
| CD95 CH11 | | (Keratinocytes) none | |
| LAK cells rest | 18.9 | CCD1106 | 34.4 |
| | | (Keratinocytes) | |
| | | TNF alpha + IL-1beta | |
| LAK cells IL-2 | 41.2 | Liver cirrhosis | 2.9 |
| LAK cells IL-2 + IL-12 | 29.5 | Lupus kidney | 2.2 |
| LAK cells IL-2 + IFN | 36.3 | NCI-H292 none | 38.4 |
| gamma | | | |
| LAK cells IL-2 + IL-18 | 34.2 | NCI-H292 IL-4 | 66.9 |
| LAK cells | 11.8 | NCI-H292 IL-9 | 62.4 |
| PMA/ionomycin | | | |
| NK Cells IL-2 rest | 29.3 | NCI-H292 IL-13 | 65.1 |
| Two Way MLR 3 day | 21.9 | NCI-H292 IFN gamma | 48.3 |
| Two Way MLR 5 day | 27.7 | HPAEC none | 31.2 |
| Two Way MLR 7 day | 27.0 | HPAEC TNF alpha + IL- | 37.6 |
| | | 1beta | |
| PBMC rest | 6.5 | Lung fibroblast none | 35.6 |
| PBMC PWM | 89.5 | Lung fibroblast TNF | 20.7 |
| | | alpha + IL-1beta | |
| PBMC PHA-L | 53.6 | Lung fibroblast IL-4 | 63.3 |
| Ramos (B cell) none | 40.6 | Lung fibroblast IL-9 | 55.5 |
| Ramos (B cell) | 56.3 | Lung fibroblast IL-13 | 44.8 |
| ionomycin | | | |
| B lymphocytes PWM | 100.0 | Lung fibroblast IFN | 71.2 |
| | | gamma | |
| B lymphocytes CD40L | 41.2 | Dermal fibroblast | 78.5 |
| and IL-4 | | CCD1070 rest | |
| EOL-1 dbcAMP | 50.0 | Dermal fibroblast | 88.9 |
| | | CCD1070 TNF alpha | |
| EOL-1 dbcAMP | 46.3 | Dermal fibroblast | 49.7 |
| PMA/ionomycin | | CCD1070 IL-1beta | |
| Dendritic cells none | 33.2 | Dermal fibroblast IFN | 21.5 |
| | | gamma | |
| Dendritic cells LPS | 26.1 | Dermal fibroblast IL-4 | 43.8 |
| Dendritic cells anti- | 29.9 | IBD Colitis 2 | 1.2 |
| CD40 | | | |
| Monocytes rest | 17.1 | IBD Crohn's | 1.8 |
| Monocytes LPS | 14.0 | Colon | 15.4 |
| Macrophages rest | 59.0 | Lung | 16.6 |
| Macrophages LPS | 29.1 | Thymus | 15.6 |
| HUVEC none | 35.1 | Kidney | 18.9 |
| HUVEC starved | 62.0 | | |

CNS_neurodegeneration_v1.0 Summary: Ag3282—This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3282 Highest expression of this gene is seen in a brain cancer cell line (CT=24.3). This gene appears to be expressed more highly in the cancer cell lines than in the normal tissue samples on this panel and may be involved in cellular growth and proliferation. Based on this expression profile, this gene may be involved in gastric, pancreatic, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

This gene is also expressed at high levels in all regions of the CNS examined. Therefore, this gene may play a role in central nervous system disorders such as Alzheimer's disease, Parkinson's disease, epilepsy, multiple sclerosis, schizophrenia and depression.

In addition, this gene product is expressed in adipose, pancreas, adrenal, thyroid, pituitary, fetal skeletal muscle, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

Furthermore, this gene is more highly expressed in fetal skeletal muscle (CT=30.4) and liver (CT=27) when compared to expression in the adult skeletal muscle (CT>35) and liver (CT=30) may be useful for the differentiation of the fetal and adult sources of this tissue.

Panel 4D Summary: Ag3282 This gene is expressed at high to moderate levels in a wide range of cell types of significance in the immune response in health and disease. Highest expression is seen in polkweed mitogen stimulated B lymphocytes (CT=25.7). In addition, expression is seen in members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in Panel 1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AA. CG57411-01: Kelch-Like Protein KLHL3C

Expression of gene CG57411-01 was assessed using the primer-probe set Ag3229, described in Table AAA. Results of the RTQ-PCR runs are shown in Tables AAB, AAC, AAD and AAE.

Table AAA. Probe Name Ag3229

TABLE AAA
Probe Name Ag3229

| | | | Start | SEQ ID |
|---|---|---|---|---|
| Primers | Sequences | Length | Position | NO: |
| Forward | 5′-gcagcgagctctaccacat-3′ | 19 | 287 | 472 |
| Probe | TET-5′-aaggccttcgcgctgcagatctt-3′-TAMRA | 23 | 310 | 473 |
| Reverse | 5′-aagtcgtccttggagatgct-3′ | 20 | 364 | 474 |

TABLE AAB
CNS_neurodegeneration_v1.0

| | Rel. Exp. (%) Ag3229, | | Rel. Exp. (%) Ag3229, |
|---|---|---|---|
| Tissue Name | Run 209862301 | Tissue Name | Run 209862301 |
| AD 1 Hippo | 16.3 | Control (Path) 3 | 8.0 |
| | | Temporal Ctx | |
| AD 2 Hippo | 34.6 | Control (Path) 4 | 35.8 |
| | | Temporal Ctx | |
| AD 3 Hippo | 15.9 | AD 1 Occipital Ctx | 18.6 |
| AD 4 Hippo | 6.9 | AD 2 Occipital Ctx | 0.0 |
| | | (Missing) | |
| AD 5 Hippo | 100.0 | AD 3 Occipital Ctx | 11.7 |
| AD 6 Hippo | 35.4 | AD 4 Occipital Ctx | 17.7 |
| Control 2 Hippo | 31.2 | AD 5 Occipital Ctx | 49.7 |
| Control 4 Hippo | 12.1 | AD 6 Occipital Ctx | 14.2 |
| Control (Path) 3 | 6.2 | Control 1 Occipital | 3.3 |
| Hippo | | Ctx | |
| AD 1 Temporal | 21.6 | Control 2 Occipital | 69.3 |
| Ctx | | Ctx | |
| AD 2 Temporal | 33.0 | Control 3 Occipital | 26.6 |
| Ctx | | Ctx | |
| AD 3 Temporal | 14.1 | Control 4 Occipital | 7.5 |
| Ctx | | Ctx | |
| AD 4 Temporal | 16.8 | Control (Path) 1 | 72.2 |
| Ctx | | Occipital Ctx | |
| AD 5 Inf Temporal | 71.7 | Control (Path) 2 | 13.7 |
| Ctx | | Occipital Ctx | |
| AD 5 Sup | 32.3 | Control (Path) 3 | 6.3 |
| Temporal Ctx | | Occipital Ctx | |
| AD 6 Inf Temporal | 30.6 | Control (Path) 4 | 16.8 |
| Ctx | | Occipital Ctx | |
| AD 6 Sup | 33.9 | Control 1 Parietal | 8.6 |
| Temporal Ctx | | Ctx | |
| Control 1 | 4.4 | Control 2 Parietal | 39.8 |
| Temporal Ctx | | Ctx | |
| Control 2 | 56.6 | Control 3 Parietal | 21.5 |
| Temporal Ctx | | Ctx | |
| Control 3 | 19.6 | Control (Path) 1 | 66.4 |
| Temporal Ctx | | Parietal Ctx | |
| Control 3 | 14.2 | Control (Path) 2 | 26.8 |
| Temporal Ctx | | Parietal Ctx | |
| Control (Path) 1 | 62.0 | Control (Path) 3 | 5.2 |
| Temporal Ctx | | Parietal Ctx | |
| Control (Path) 2 | 36.1 | Control (Path) 4 | 54.3 |
| Temporal Ctx | | Parietal Ctx | |

TABLE AAC
General_screening_panel_v1.4

| | Rel. Exp. (%) Ag3229, Run | | Rel. Exp. (%) Ag3229, Run |
|---|---|---|---|
| Tissue Name | 214439727 | Tissue Name | 214439727 |
| Adipose | 6.0 | Renal ca. TK-10 | 20.4 |
| Melanoma* | 8.1 | Bladder | 6.7 |
| Hs688(A).T | | | |
| Melanoma* | 13.5 | Gastric ca. (liver met.) | 11.2 |
| Hs688(B).T | | NCI-N87 | |
| Melanoma* M14 | 2.1 | Gastric ca. KATO III | 59.5 |
| Melanoma* | 24.8 | Colon ca. SW-948 | 0.6 |
| LOXIMVI | | | |
| Melanoma* SK- | 20.7 | Colon ca. SW480 | 31.6 |
| MEL-5 | | | |
| Squamous cell | 6.7 | Colon ca.* (SW480 | 4.7 |
| carcinoma SCC-4 | | met) SW620 | |
| Testis Pool | 3.0 | Colon ca. HT29 | 2.7 |
| Prostate ca.* (bone | 17.8 | Colon ca. HCT-116 | 35.1 |
| met) PC-3 | | | |
| Prostate Pool | 8.5 | Colon ca. CaCo-2 | 2.5 |
| Placenta | 14.2 | Colon cancer tissue | 6.2 |
| Uterus Pool | 5.8 | Colon ca. SW1116 | 3.3 |
| Ovarian ca. | 40.9 | Colon ca. Colo-205 | 5.4 |
| OVCAR-3 | | | |
| Ovarian ca. SK- | 17.7 | Colon ca. SW-48 | 3.3 |
| OV-3 | | | |
| Ovarian ca. | 11.9 | Colon Pool | 25.0 |
| OVCAR-4 | | | |
| Ovarian ca. | 84.1 | Small Intestine Pool | 14.9 |
| OVCAR-5 | | | |
| Ovarian ca. | 2.0 | Stomach Pool | 6.4 |
| IGROV-1 | | | |
| Ovarian ca. | 8.1 | Bone Marrow Pool | 9.7 |
| OVCAR-8 | | | |
| Ovary | 8.7 | Fetal Heart | 1.7 |
| Breast ca. MCF-7 | 0.9 | Heart Pool | 10.7 |
| Breast ca. MDA- | 30.1 | Lymph Node Pool | 21.8 |
| MB-231 | | | |
| Breast ca. BT 549 | 8.1 | Fetal Skeletal Muscle | 4.2 |
| Breast ca. T47D | 100.0 | Skeletal Muscle Pool | 8.7 |
| Breast ca. MDA-N | 0.0 | Spleen Pool | 10.4 |
| Breast Pool | 22.4 | Thymus pool | 11.2 |
| Trachea | 10.4 | CNS cancer | 55.5 |
| | | (glio/astro) U87-MG | |
| Lung | 1.7 | CNS cancer | 44.8 |
| | | (glio/astro) U-118-MG | |
| Fetal Lung | 6.7 | CNS cancer | 5.8 |
| | | (neuro; met) SK-N-AS | |
| Lung ca. NCI-N417 | 11.7 | CNS cancer (astro) SF- | 0.4 |
| | | 539 | |
| Lung ca. LX-1 | 37.1 | CNS cancer (astro) | 5.0 |
| | | SNB-75 | |
| Lung ca. NCI-H146 | 6.2 | CNS cancer (glio) | 2.9 |
| | | SNB-19 | |
| Lung ca. SHP-77 | 61.1 | CNS cancer (glio) SF- | 39.0 |
| | | 295 | |
| Lung ca. A549 | 6.3 | Brain (Amygdala) Pool | 8.4 |
| Lung ca. NCI-H526 | 8.7 | Brain (cerebellum) | 22.2 |
| Lung ca. NCI-H23 | 6.3 | Brain (fetal) | 48.6 |
| Lung ca. NCI-H460 | 2.9 | Brain (Hippocampus) | 8.5 |
| | | Pool | |
| Lung ca. HOP-62 | 8.1 | Cerebral Cortex Pool | 20.7 |
| Lung ca. NCI-H522 | 0.5 | Brain (Substantia | 14.7 |
| | | nigra) Pool | |
| Liver | 0.3 | Brain (Thalamus) Pool | 13.8 |
| Fetal Liver | 1.8 | Brain (whole) | 11.5 |
| Liver ca. HepG2 | 0.2 | Spinal Cord Pool | 3.3 |
| Kidney Pool | 26.8 | Adrenal Gland | 29.9 |
| Fetal Kidney | 10.2 | Pituitary gland Pool | 10.7 |
| Renal ca. 786-0 | 7.5 | Salivary Gland | 4.1 |
| Renal ca. A498 | 4.0 | Thyroid (female) | 1.1 |
| Renal ca. ACHN | 11.9 | Pancreatic ca. | 1.0 |
| | | CAPAN2 | |
| Renal ca. UO-31 | 15.3 | Pancreas Pool | 28.7 |

TABLE AAD
Panel 2.2

| | Rel. Exp. (%) Ag3229, Run | | Rel. Exp. (%) Ag3229, Run |
|---|---|---|---|
| Tissue Name | 174442765 | Tissue Name | 174442765 |
| Normal Colon | 15.5 | Kidney Margin | 100.0 |
| | | (OD04348) | |
| Colon cancer | 31.9 | Kidney malignant | 10.7 |
| (OD06064) | | cancer (OD06204B) | |
| Colon Margin | 20.6 | Kidney normal | 11.6 |
| (OD06064) | | adjacent tissue | |
| | | (OD06204E) | |
| Colon cancer | 6.0 | Kidney Cancer | 38.4 |
| (OD06159) | | (OD04450-01) | |
| Colon Margin | 12.7 | Kidney Margin | 17.4 |
| (OD06159) | | (OD04450-03) | |
| Colon cancer | 3.7 | Kidney Cancer | 0.0 |
| (OD06297-04) | | 8120613 | |
| Colon Margin | 22.4 | Kidney Margin | 6.0 |
| (OD06297-05) | | 8120614 | |
| CC Gr.2 ascend colon | 6.5 | Kidney Cancer | 12.0 |
| (ODO3921) | | 9010320 | |
| CC Margin (ODO3921) | 10.5 | Kidney Margin | 9.9 |
| | | 9010321 | |
| Colon cancer metastasis | 8.6 | Kidney Cancer | 47.3 |
| (OD06104) | | 8120607 | |
| Lung Margin | 6.2 | Kidney Margin | 5.6 |
| (OD06104) | | 8120608 | |
| Colon mets to lung | 31.0 | Normal Uterus | 48.3 |
| (OD04451-01) | | | |
| Lung Margin | 39.5 | Uterine Cancer 064011 | 14.9 |
| (OD04451-02) | | | |
| Normal Prostate | 41.2 | Normal Thyroid | 2.6 |
| Prostate Cancer | 8.1 | Thyroid Cancer | 4.3 |
| (OD04410) | | 064010 | |
| Prostate Margin | 10.6 | Thyroid Cancer | 15.3 |
| (OD04410) | | A302152 | |
| Normal Ovary | 23.2 | Thyroid Margin | 2.7 |
| | | A302153 | |
| Ovarian cancer | 7.2 | Normal Breast | 46.0 |
| (OD06283-03) | | | |
| Ovarian Margin | 17.8 | Breast Cancer | 5.9 |
| (OD06283-07) | | (OD04566) | |
| Ovarian Cancer 064008 | 22.2 | Breast Cancer 1024 | 27.4 |
| Ovarian cancer | 9.0 | Breast Cancer | 19.5 |
| (OD06145) | | (OD04590-01) | |
| Ovarian Margin | 13.4 | Breast Cancer Mets | 13.5 |
| (OD06145) | | (OD04590-03) | |
| Ovarian cancer | 6.3 | Breast Cancer | 12.2 |
| (OD06455-03) | | Metastasis (OD04655- | |
| | | 05) | |
| Ovarian Margin | 12.9 | Breast Cancer 064006 | 8.1 |
| (OD06455-07) | | | |
| Normal Lung | 14.5 | Breast Cancer 9100266 | 3.0 |
| Invasive poor diff. lung | 5.0 | Breast Margin | 3.4 |
| adeno (ODO4945-01) | | 9100265 | |
| Lung Margin | 37.4 | Breast Cancer | 11.2 |
| (ODO4945-03) | | A209073 | |
| Lung Malignant Cancer | 9.6 | Breast Margin | 61.1 |
| (OD03126) | | A2090734 | |
| Lung Margin | 14.2 | Breast cancer | 4.7 |
| (OD03126) | | (OD06083) | |
| Lung Cancer | 4.9 | Breast cancer node | 12.7 |
| (OD05014A) | | metastasis (OD06083) | |
| Lung Margin | 39.0 | Normal Liver | 2.8 |
| (OD05014B) | | | |
| Lung cancer | 17.4 | Liver Cancer 1026 | 13.6 |
| (OD06081) | | | |
| Lung Margin | 32.3 | Liver Cancer 1025 | 12.9 |
| (OD06081) | | | |
| Lung Cancer | 4.2 | Liver Cancer 6004-T | 13.2 |
| (OD04237-01) | | | |
| Lung Margin | 24.7 | Liver Tissue 6004-N | 1.3 |
| (OD04237-02) | | | |
| Ocular Melanoma | 12.9 | Liver Cancer 6005-T | 43.2 |
| Metastasis | | | |
| Ocular Melanoma | 10.7 | Liver Tissue 6005-N | 4.8 |
| Margin (Liver) | | | |
| Melanoma Metastasis | 52.9 | Liver Cancer 064003 | 39.5 |
| Melanoma Margin | 21.0 | Normal Bladder | 9.3 |
| (Lung) | | | |
| Normal Kidney | 4.7 | Bladder Cancer 1023 | 5.8 |
| Kidney Ca, Nuclear | 40.3 | Bladder Cancer | 4.2 |
| grade 2 (OD04338) | | A302173 | |
| Kidney Margin | 7.5 | Normal Stomach | 31.4 |
| (OD04338) | | | |
| Kidney Ca Nuclear | 82.4 | Gastric Cancer | 1.2 |
| grade 1/2 (OD04339) | | 9060397 | |
| Kidney Margin | 13.2 | Stomach Margin | 7.1 |
| (OD04339) | | 9060396 | |
| Kidney Ca, Clear cell | 8.3 | Gastric Cancer | 7.4 |
| type (OD04340) | | 9060395 | |
| Kidney Margin | 24.7 | Stomach Margin | 10.9 |
| (OD04340) | | 9060394 | |
| Kidney Ca, Nuclear | 13.1 | Gastric Cancer 064005 | 10.4 |
| grade 3 (OD04348) | | | |

TABLE AAE
Panel 4D

| | Rel. Exp. (%) | | Rel. Exp. (%) |
|---|---|---|---|
| | Ag3229, Run | | Ag3229, Run |
| Tissue Name | 164389704 | Tissue Name | 164389704 |
| Secondary Th1 act | 3.4 | HUVEC IL-1beta | 20.0 |
| Secondary Th2 act | 4.8 | HUVEC IFN gamma | 32.5 |
| Secondary Tr1 act | 2.1 | HUVEC TNF alpha + | 26.6 |
| | | IFN gamma | |
| Secondary Th1 rest | 1.2 | HUVEC TNF alpha + | 35.1 |
| | | IL4 | |
| Secondary Th2 rest | 2.0 | HUVEC IL-11 | 17.6 |
| Secondary Tr1 rest | 4.5 | Lung Microvascular EC | 34.2 |
| | | none | |
| Primary Th1 act | 17.7 | Lung Microvascular EC | 49.0 |
| | | TNF alpha + IL-1beta | |
| Primary Th2 act | 5.3 | Microvascular Dermal | 30.6 |
| | | EC none | |
| Primary Tr1 act | 25.9 | Microsvasular Dermal | 38.7 |
| | | EC TNF alpha + IL-1beta | |
| Primary Th1 rest | 14.0 | Bronchial epithelium | 46.7 |
| | | TNF alpha + IL1beta | |
| Primary Th2 rest | 6.5 | Small airway epithelium | 22.1 |
| | | none | |
| Primary Tr1 rest | 22.1 | Small airway epithelium | 97.9 |
| | | TNF alpha + IL-1beta | |
| CD45RA CD4 | 12.7 | Coronery artery SMC rest | 31.2 |
| lymphocyte act | | | |
| CD45RO CD4 | 6.6 | Coronery artery SMC | 10.5 |
| lymphocyte act | | TNF alpha + IL-1beta | |
| CD8 lymphocyte act | 2.4 | Astrocytes rest | 7.5 |
| Secondary CD8 | 4.2 | Astrocytes TNF alpha + | 8.6 |
| lymphocyte rest | | IL-1beta | |
| Secondary CD8 | 0.0 | KU-812 (Basophil) rest | 0.8 |
| lymphocyte act | | | |
| CD4 lymphocyte none | 5.6 | KU-812 (Basophil) | 2.9 |
| | | PMA/ionomycin | |
| 2ry Th1/Th2/Tr1_anti- | 3.7 | CCD1106 | 6.2 |
| CD95 CH11 | | (Keratinocytes) none | |
| LAK cells rest | 5.9 | CCD1106 | 6.0 |
| | | (Keratinocytes) | |
| | | TNF alpha + IL-1beta | |
| LAK cells IL-2 | 3.0 | Liver cirrhosis | 15.5 |
| LAK cells IL-2 + IL-12 | 6.2 | Lupus kidney | 12.2 |
| LAK cells IL-2 + IFN | 10.7 | NCI-H292 none | 30.8 |
| gamma | | | |
| LAK cells IL-2 + IL-18 | 5.0 | NCI-H292 IL-4 | 49.7 |
| LAK cells | 4.0 | NCI-H292 IL-9 | 43.5 |
| PMA/ionomycin | | | |
| NK Cells IL-2 rest | 1.9 | NCI-H292 IL-13 | 31.6 |
| Two Way MLR 3 day | 9.0 | NCI-H292 IFN gamma | 17.7 |
| Two Way MLR 5 day | 3.3 | HPAEC none | 18.0 |
| Two Way MLR 7 day | 1.2 | HPAEC TNF alpha + IL- | 58.2 |
| | | 1beta | |
| PBMC rest | 0.8 | Lung fibroblast none | 40.6 |
| PBMC PWM | 10.7 | Lung fibroblast TNF | 11.0 |
| | | alpha + IL-1beta | |
| PBMC PHA-L | 10.2 | Lung fibroblast IL-4 | 100.0 |
| Ramos (B cell) none | 0.0 | Lung fibroblast IL-9 | 55.1 |
| Ramos (B cell) | 0.0 | Lung fibroblast IL-13 | 78.5 |
| ionomycin | | | |
| B lymphocytes PWM | 23.3 | Lung fibroblast IFN | 82.4 |
| | | gamma | |
| B lymphocytes CD40L | 18.6 | Dermal fibroblast | 45.4 |
| and IL-4 | | CCD1070 rest | |
| EOL-1 dbcAMP | 1.8 | Dermal fibroblast | 36.3 |
| | | CCD1070 TNF alpha | |
| EOL-1 dbcAMP | 2.0 | Dermal fibroblast | 23.8 |
| PMA/ionomycin | | CCD1070 IL-1beta | |
| Dendritic cells none | 5.9 | Dermal fibroblast IFN | 4.6 |
| | | gamma | |
| Dendritic cells LPS | 8.0 | Dermal fibroblast IL-4 | 16.6 |
| Dendritic cells anti- | 3.3 | IBD Colitis 2 | 6.2 |
| CD40 | | | |
| Monocytes rest | 4.2 | IBD Crohn's | 3.6 |
| Monocytes LPS | 0.9 | Colon | 30.1 |
| Macrophages rest | 3.1 | Lung | 19.9 |
| Macrophages LPS | 1.4 | Thymus | 20.6 |
| HUVEC none | 35.1 | Kidney | 20.9 |
| HUVEC starved | 58.2 | | |

CNS_neurodegeneration_v1.0 Summary: A3229—This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3229—Highest levels of expression of this gene are seen in breast cancer cell line T47D (CT=28.5). Based on expression in this panel, this gene may be involved in gastric, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

This gene product is also expressed in adipose, pancreas, adrenal, thyroid, pituitary, skeletal muscle, and heart. This widespread expression in tissues with metabolic function suggests, that this gene product may be important for the pathogenesis, diagnosis, and/or treatment of metabolic and endocrine diseases, including obesity and Types 1 and 2 diabetes.

In addition, this gene is expressed at low to moderate levels in all regions of the CNS examined. Therefore, this gene may play a role in central nervous system disorders such as Alzheimer's disease, Parkinson's disease, epilepsy, multiple sclerosis, schizophrenia and depression.

Panel 2.2 Summary: Ag3229 Highest expression of the CG57411-01 gene is seen in the kidney (CT=32.2). In addition, significant levels of expression are seen in samples derived from normal lung and breast. Expression in these normal tissues is also higher than in the corresponding malignant tissue. Thus, expression of this gene could be used to differentiate between these samples and other samples on this panel and as a marker to detect the presence of lung, breast and kidney cancer. Furthermore, therapeutic modulation of the expression or function of this gene may be effective in the treatment of lung, breast and kidney cancer.

Panel 4D Summary: Ag3229 Highest expression of the CG57411-01 gene is seen in IL-4 treated lung fibroblasts (CT=31.3). Significant levels of expression are seen in activated-NCI-H292 mucoepidermoid cells as well as untreated NCI-H292 cells. Moderate expression is also detected in IL-9, IL-13 and IFN gamma activated lung fibroblasts, human pulmonary aortic endothelial cells (treated and untreated), small airway epithelium (treated and untreated), treated bronchial epithelium and lung microvascular endothelial cells (treated and untreated). The expression of this gene in cells derived from or within the lung suggests that this gene may be involved in normal conditions as well as pathological and inflammatory lung disorders that include chronic obstructive pulmonary disease, asthma, allergy and emphysema. Moderate/low expression of this gene is also detected in treated and untreated HUVECs (endothelial cells) and coronary artery smooth muscle cells (treated and untreated) and normal tissues that include lung, colon, thymus and kidney. Expression in the various immune cell types and tissue samples suggests that therapeutic modulation of this gene product may ameliorate symptoms associated with infectious conditions as well as inflammatory and autoimmune disorders that include psoriasis, allergy, asthma, inflammatory bowel disease, rheumatoid arthritis and osteoarthritis.

AB. CG57399-01 and CG57399-03: Phospholipase ADRAB-B Precursor

Expression of gene CG57399-01 and variant CG57399-03 was assessed using the primer-probe sets Ag3952 and Ag3226, described in Tables ABA and ABB. Results of the RTQ-PCR runs are shown in Tables ABC and ABD.

TABLE ABA
Probe Name Ag3952

| | | | Start | SEQ ID |
|---|---|---|---|---|
| Primers | Sequences | Length | Position | NO: |
| Forward | 5′-ctgtgtccctgtgtcctgaa-3′ | 20 | 1633 | 475 |
| Probe | TET-5′-tcaacagaacttgctaccctcatcga-3′-TAMRA | 26 | 1666 | 476 |
| Reverse | 5′-gtgggtcttctcctgaaacttc-3′ | 22 | 1701 | 477 |

TABLE ABB
Probe Name Ag3226

| | | | Start | SEQ ID |
|---|---|---|---|---|
| Primers | Sequences | Length | Position | NO: |
| Forward | 5′-gatgatcctcaggtcactgtgt-3′ | 22 | 1617 | 478 |
| Probe | TET-5′-ccctgtgtcctgaagtttgatgataactca-3′-TAMRA | 30 | 1639 | 479 |
| Reverse | 5′-tcgatgagggtagcaagttct-3′ | 21 | 1671 | 480 |

TABLE ABC
General_screening_panel_v1.4

| | Rel. Exp. (%) Ag3952, Run | | Rel. Exp. (%) Ag3952, Run |
|---|---|---|---|
| Tissue Name | 213856126 | Tissue Name | 213856126 |
| Adipose | 9.0 | Renal ca. TK-10 | 15.0 |
| Melanoma* | 3.0 | Bladder | 22.7 |
| Hs688(A).T | | | |
| Melanoma* | 3.4 | Gastric ca. (liver met.) | 13.0 |
| Hs688(B).T | | NCI-N87 | |
| Melanoma* M14 | 0.9 | Gastric ca. KATO III | 75.3 |
| Melanoma* | 11.7 | Colon ca. SW-948 | 4.3 |
| LOXIMVI | | | |
| Melanoma* SK- | 1.5 | Colon ca. SW480 | 97.3 |
| MEL-5 | | | |
| Squamous cell | 8.7 | Colon ca.* (SW480 | 4.4 |
| carcinoma SCC-4 | | met) SW620 | |
| Testis Pool | 12.8 | Colon ca. HT29 | 0.4 |
| Prostate ca.* (bone | 10.5 | Colon ca. HCT-116 | 1.2 |
| met) PC-3 | | | |
| Prostate Pool | 12.9 | Colon ca. CaCo-2 | 60.7 |
| Placenta | 5.1 | Colon cancer tissue | 28.7 |
| Uterus Pool | 6.5 | Colon ca. SW1116 | 0.0 |
| Ovarian ca. | 7.3 | Colon ca. Colo-205 | 0.9 |
| OVCAR-3 | | | |
| Ovarian ca. SK- | 26.4 | Colon ca. SW-48 | 26.1 |
| OV-3 | | | |
| Ovarian ca. | 1.9 | Colon Pool | 18.8 |
| OVCAR-4 | | | |
| Ovarian ca. | 6.7 | Small Intestine Pool | 5.3 |
| OVCAR-5 | | | |
| Ovarian ca. | 9.2 | Stomach Pool | 7.9 |
| IGROV-1 | | | |
| Ovarian ca. | 4.2 | Bone Marrow Pool | 8.4 |
| OVCAR-8 | | | |
| Ovary | 10.0 | Fetal Heart | 1.2 |
| Breast ca. MCF-7 | 0.4 | Heart Pool | 5.7 |
| Breast ca. MDA- | 92.0 | Lymph Node Pool | 32.1 |
| MB-231 | | | |
| Breast ca. BT 549 | 5.5 | Fetal Skeletal Muscle | 1.2 |
| Breast ca. T47D | 2.5 | Skeletal Muscle Pool | 4.7 |
| Breast ca. MDA-N | 1.6 | Spleen Pool | 18.2 |
| Breast Pool | 19.6 | Thymus Pool | 19.3 |
| Trachea | 10.3 | CNS cancer | 38.2 |
| | | (glio/astro) U87-MG | |
| Lung | 1.2 | CNS cancer | 12.2 |
| | | (glio/astro) U-118-MG | |
| Fetal Lung | 8.3 | CNS cancer | 0.9 |
| | | (neuro; met) SK-N-AS | |
| Lung ca. NCI-N417 | 0.9 | CNS cancer (astro) SF- | 7.6 |
| | | 539 | |
| Lung ca. LX-1 | 27.2 | CNS cancer (astro) | 17.1 |
| | | SNB-75 | |
| Lung ca. NCI-H146 | 10.7 | CNS cancer (glio) | 6.8 |
| | | SNB-19 | |
| Lung ca. SHP-77 | 47.3 | CNS cancer (glio) SF- | 5.7 |
| | | 295 | |
| Lung ca. A549 | 5.1 | Brain (Amygdala) Pool | 7.0 |
| Lung ca. NCI-H526 | 0.0 | Brain (cerebellum) | 3.2 |
| Lung ca. NCI-H23 | 4.1 | Brain (fetal) | 19.3 |
| Lung ca. NCI-H460 | 0.5 | Brain (Hippocampus) | 13.1 |
| | | Pool | |
| Lung ca. HOP-62 | 2.7 | Cerebral Cortex Pool | 14.8 |
| Lung ca. NCI-H522 | 1.3 | Brain (Substantia | 6.3 |
| | | nigra) Pool | |
| Liver | 0.0 | Brain (Thalamus) Pool | 15.2 |
| Fetal Liver | 1.7 | Brain (whole) | 10.4 |
| Liver ca. HepG2 | 0.5 | Spinal Cord Pool | 5.3 |
| Kidney Pool | 21.2 | Adrenal Gland | 100.0 |
| Fetal Kidney | 1.6 | Pituitary gland Pool | 4.3 |
| Renal ca. 786-0 | 1.7 | Salivary Gland | 3.4 |
| Renal ca. A498 | 1.3 | Thyroid (female) | 14.5 |
| Renal ca. ACHN | 4.3 | Pancreatic ca. | 1.7 |
| | | CAPAN2 | |
| Renal ca. UO-31 | 17.4 | Pancreas Pool | 24.5 |

TABLE ABD
Panel 1.3D

| | Rel. Exp. (%) Ag3226, Run | | Rel. Exp. (%) Ag3226, Run |
|---|---|---|---|
| Tissue Name | 167994701 | Tissue Name | 167994701 |
| Liver adenocarcinoma | 2.5 | Kidney (fetal) | 16.3 |
| Pancreas | 0.0 | Renal ca. 786-0 | 0.9 |
| Pancreatic ca. CAPAN2 | 0.0 | Renal ca. A498 | 1.4 |
| Adrenal gland | 19.6 | Renal ca. RXF 393 | 3.4 |
| Thyroid | 16.3 | Renal ca. ACHN | 1.4 |
| Salivary gland | 0.0 | Renal ca. UO-31 | 2.8 |
| Pituitary gland | 1.9 | Renal ca. TK-10 | 4.4 |
| Brain (fetal) | 25.5 | Liver | 0.0 |
| Brain (whole) | 4.6 | Liver (fetal) | 1.1 |
| Brain (amygdala) | 6.7 | Liver ca. | 0.0 |
| | | (hepatoblast) HepG2 | |
| Brain (cerebellum) | 1.6 | Lung | 8.8 |
| Brain (hippocampus) | 22.2 | Lung (fetal) | 1.7 |
| Brain (substantia nigra) | 3.1 | Lung ca. (small cell) | 18.6 |
| | | LX-1 | |
| Brain (thalamus) | 3.2 | Lung ca. (small cell) | 4.2 |
| | | NCI-H69 | |
| Cerebral Cortex | 26.2 | Lung ca. (s.cell var.) | 100.0 |
| | | SHP-77 | |
| Spinal cord | 3.1 | Lung ca. (large | 0.0 |
| | | cell)NCI-H460 | |
| glio/astro U87-MG | 7.5 | Lung ca. (non-sm. | 6.7 |
| | | cell) A549 | |
| glio/astro U-118-MG | 4.2 | Lung ca. (non-s.cell) | 5.7 |
| | | NCI-H23 | |
| astrocytoma SW1783 | 1.2 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.9 |
| | | SW 900 | |
| astrocytoma SNB-75 | 4.3 | Lung ca. (squam.) | 3.7 |
| | | NCI-H596 | |
| glioma SNB-19 | 6.0 | Mammary gland | 6.3 |
| glioma U251 | 14.1 | Breast ca.* (pl.ef) | 0.0 |
| | | MCF-7 | |
| glioma SF-295 | 0.0 | Breast ca.* (pl.ef) | 45.4 |
| | | MDA-MB-231 | |
| Heart (fetal) | 1.4 | Breast ca.* (pl.ef) | 4.3 |
| | | T47D | |
| Heart | 1.0 | Breast ca. BT-549 | 7.1 |
| Skeletal muscle (fetal) | 0.7 | Breast ca. MDA-N | 0.0 |
| Skeletal muscle | 3.2 | Ovary | 10.9 |
| Bone marrow | 3.1 | Ovarian ca. | 0.0 |
| | | OVCAR-3 | |
| Thymus | 5.7 | Ovarian ca. | 2.4 |
| | | OVCAR-4 | |
| Spleen | 7.2 | Ovarian ca. | 5.2 |
| | | OVCAR-5 | |
| Lymph node | 0.0 | Ovarian ca. | 0.0 |
| | | OVCAR-8 | |
| Colorectal | 4.8 | Ovarian ca. IGROV-1 | 0.0 |
| Stomach | 5.1 | Ovarian ca.* | 3.0 |
| | | (ascites) SK-OV-3 | |
| Small intestine | 1.5 | Uterus | 5.8 |
| Colon ca. SW480 | 33.2 | Placenta | 0.0 |
| Colon ca.* | 8.8 | Prostate | 1.6 |
| SW620(SW480 met) | | | |
| Colon ca. HT29 | 0.0 | Prostate ca.* (bone | 2.6 |
| | | met)PC-3 | |
| Colon ca. HCT-116 | 0.0 | Testis | 7.4 |
| Colon ca. CaCo-2 | 35.4 | Melanoma | 0.0 |
| | | Hs688(A).T | |
| Colon ca. | 24.5 | Melanoma* (met) | 0.0 |
| tissue(ODO3866) | | Hs688(B).T | |
| Colon ca. HCC-2998 | 15.7 | Melanoma UACC- | 0.0 |
| | | 62 | |
| Gastric ca.* (liver met) | 6.4 | Melanoma M14 | 0.0 |
| NCI-N87 | | | |
| Bladder | 14.6 | Melanoma LOX | 0.0 |
| | | IMVI | |
| Trachea | 4.4 | Melanoma* (met) | 0.0 |
| | | SK-MEL-5 | |
| Kidney | 2.4 | Adipose | 17.3 |

General_screening_panel_v1.4 Summary: Ag3952 Highest expression of this gene is seen in the adrenal gland (CT=29). Thus, this gene product may be a treatment for Addison's disease and other adrenalopathies. This gene also has low levels of expression in adipose, heart, skeletal muscle, pituitary, thyroid, and pancreas. Therapeutic modulation of this gene product may be important for the diagnosis or treatment of endocrine or metabolic disease, including Types 1 and 2 diabetes, obesity and pancreatitis.

Expression of this gene is also seen in sample derived from colon, gastric, lung and breast cancers. Thus, expression of this gene could be used to differentiate between these samples and other samples on this panel and as a marker to detect the presence of these cancers. Furthermore, therapeutic modulation of the expression or function of this gene may be effective in the treatment of colon, gastric, lung and breast cancers.

Low but significant levels of expression are also seen for all regions of the CNS examined. Thus, this gene product may be useful for treatment of CNS disorders such as Alzheimner's disease, Parkinson's disease, stroke, epilepsy, schizophrenia and multiple sclerosis.

Panel 1.3D Summary: Ag3952 Highest expression of the CG57399-01 gene is seen in a lung cancer cell line (CT=32.5). Low but significant expression is also seen in cell lines derived from breast and colon cancers. Overall, expression is consistent with expression seen in Panel 1.4. Thus, expression of this gene could be used to differentiate between these samples and other samples on this panel and as a marker to detect the presence of these cancers. Furthermore, therapeutic modulation of the expression or function of this gene may be effective in the treatment of colon, gastric, lung and breast cancers.

Among metabolic tissues, significant levels of expression are seen in adipose and the adrenal gland. Thus, this gene product may be useful for treatment of obesity, Addisonl's disease and other adrenalopathies.

In addition, this gene is expressed in the hippocampus, and cerebral cortex. Both these regions of the brain undergo degeneration in Alzheimer's disease. Thus, therapeutic modulation of the expression or function of this gene may be effective in the treatment of this disease or any other neurodegenerative disorders.

AC. CG57399-02: Phospholipase Adrab-B Precursor

Expression of gene CG57399-02 was assessed using the primer-probe set Ag3952, described in Table ACA. Results of the RTQ-PCR runs are shown in Table ACB. Please note that this gene represents a variant of CG57399-01. This sequence however, only corresponds to probe and primer set Ag3952.

TABLE ACA
Probe Name Ag3952

| | | | | SEQ ID |
|---|---|---|---|---|
| Primers | Sequences | Length | Start Position | NO: |
| Forward | 5′-ctgtgtccctgtgtcctgaa-3′ | 20 | 578 | 481 |
| Probe | TET-5′-tcaacagaacttgctaccctcatcga-3′-TAMRA | 26 | 611 | 482 |
| Reverse | 5′-gtgggtcttctcctgaaacttc-3′ | 22 | 646 | 483 |

TABLE ACB
General_screening_panel_v1.4

| | Rel. Exp. (%) Ag3952, Run | | Rel. Exp. (%) Ag3952, Run |
|---|---|---|---|
| Tissue Name | 213856126 | Tissue Name | 213856126 |
| Adipose | 9.0 | Renal ca. TK-10 | 15.0 |
| Melanoma* | 3.0 | Bladder | 22.7 |
| Hs688(A).T | | | |
| Melanoma* | 3.4 | Gastric ca. (liver met.) | 13.0 |
| Hs688(B).T | | NCI-N87 | |
| Melanoma* M14 | 0.9 | Gastric ca. KATO III | 75.3 |
| Melanoma* | 11.7 | Colon ca. SW-948 | 4.3 |
| LOXIMVI | | | |
| Melanoma* SK- | 1.5 | Colon ca. SW480 | 97.3 |
| MEL-5 | | | |
| Squamous cell | 8.7 | Colon ca.* (SW480 | 4.4 |
| carcinoma SCC-4 | | met) SW620 | |
| Testis Pool | 12.8 | Colon ca. HT29 | 0.4 |
| Prostate ca.* (bone | 10.5 | Colon ca. HCT-116 | 1.2 |
| met) PC-3 | | | |
| Prostate Pool | 12.9 | Colon ca. CaCo-2 | 60.7 |
| Placenta | 5.1 | Colon cancer tissue | 28.7 |
| Uterus Pool | 6.5 | Colon ca. SW1116 | 0.0 |
| Ovarian ca. | 7.3 | Colon ca. Colo-205 | 0.9 |
| OVCAR-3 | | | |
| Ovarian ca. SK- | 26.4 | Colon ca. SW-48 | 26.1 |
| OV-3 | | | |
| Ovarian ca. | 1.9 | Colon Pool | 18.8 |
| OVCAR-4 | | | |
| Ovarian ca. | 6.7 | Small Intestine Pool | 5.3 |
| OVCAR-5 | | | |
| Ovarian ca. | 9.2 | Stomach Pool | 7.9 |
| IGROV-1 | | | |
| Ovarian ca. | 4.2 | Bone Marrow Pool | 8.4 |
| OVCAR-8 | | | |
| Ovary | 10.0 | Fetal Heart | 1.2 |
| Breast ca. MCF-7 | 0.4 | Heart Pool | 5.7 |
| Breast ca. MDA- | 92.0 | Lymph Node Pool | 32.1 |
| MB-231 | | | |
| Breast ca. BT 549 | 5.5 | Fetal Skeletal Muscle | 1.2 |
| Breast ca. T47D | 2.5 | Skeletal Muscle Pool | 4.7 |
| Breast ca. MDA-N | 1.6 | Spleen Pool | 18.2 |
| Breast Pool | 19.6 | Thymus Pool | 19.3 |
| Trachea | 10.3 | CNS cancer | 38.2 |
| | | (glio/astro) U87-MG | |
| Lung | 1.2 | CNS cancer | 12.2 |
| | | (glio/astro) U-118-MG | |
| Fetal Lung | 8.3 | CNS cancer | 0.9 |
| | | (neuro; met) SK-N-AS | |
| Lung ca. NCI-N417 | 0.9 | CNS cancer (astro) SF- | 7.6 |
| | | 539 | |
| Lung ca. LX-1 | 27.2 | CNS cancer (astro) | 17.1 |
| | | SNB-75 | |
| Lung ca. NCI-H146 | 10.7 | CNS cancer (glio) | 6.8 |
| | | SNB-19 | |
| Lung ca. SHP-77 | 47.3 | CNS cancer (glio) SF- | 5.7 |
| | | 295 | |
| Lung ca. A549 | 5.1 | Brain (Amygdala) Pool | 7.0 |
| Lung ca. NCI-H526 | 0.0 | Brain (cerebellum) | 3.2 |
| Lung ca. NCI-H23 | 4.1 | Brain (fetal) | 19.3 |
| Lung ca. NCI-H460 | 0.5 | Brain (Hippocampus) | 13.1 |
| | | Pool | |
| Lung ca. HOP-62 | 2.7 | Cerebral Cortex Pool | 14.8 |
| Lung ca. NCI-H522 | 1.3 | Brain (Substantia | 6.3 |
| | | nigra) Pool | |
| Liver | 0.0 | Brain (Thalamus) Pool | 15.2 |
| Fetal Liver | 1.7 | Brain (whole) | 10.4 |
| Liver ca. HepG2 | 0.5 | Spinal Cord Pool | 5.3 |
| Kidney Pool | 21.2 | Adrenal Gland | 100.0 |
| Fetal Kidney | 1.6 | Pituitary gland Pool | 4.3 |
| Renal ca. 786-0 | 1.7 | Salivary Gland | 3.4 |
| Renal ca. A498 | 1.3 | Thyroid (female) | 14.5 |
| Renal ca. ACHN | 4.3 | Pancreatic ca. | 1.7 |
| | | CAPAN2 | |
| Renal ca. UO-31 | 17.4 | Pancreas Pool | 24.5 |

General_screening_panel_v1.4 Summary: Ag3952 Highest expression of this gene is seen in the adrenal gland (CT=29). Thus, this gene product may be a treatment for Addison's disease and other adrenalopathies. This gene also has low levels of expression in adipose., heart, skeletal muscle, pituitary, thyroid, and pancreas. Therapeutic modulation of this gene product may be important for the diagnosis or treatment of endocrine or metabolic disease, including Types 1 and 2 diabetes, obesity and pancreatitis.

Expression of this gene is also seen in cell line samples derived from colon, gastric, lung and breast cancers. Thus, expression of this gene could be used to differentiate between these samples and other samples on this panel and as a marker to detect the presence of these cancers. Furthermore, therapeutic modulation of the expression or function of this gene may be effective in the treatment of colon, gastric, lung and breast cancers.

Low but significant levels of expression are also seen for all regions of the CNS examined. Thus, this gene product may be useful for treatment of CNS disorders such as Alzheimer's disease, Parkinson's disease, stroke, epilepsy, schizophrenia and multiple sclerosis.

AD. CG59311-01: ACYL-Coenzyme a Thioester Hydrolase bp.

Expression of gene CG59311-01, splice variant CG59311-02, and full length clone CG59311-03, was assessed using the primer-probe set Ag3541, described in Table ADA. Results of the RTQ-PCR runs are shown in Tables ADB and ADC.

Table ADA. Probe Name Ag3541

TABLE ADA
Probe Name Ag3541

| | | | Start | SEQ ID |
|---|---|---|---|---|
| Primer | Sequences | Length | Position | NO: |
| Forward | 5′-ctcactcaaaggcacaggtaga-3′ | 22 | 1199 | 484 |
| Probe | TET-5′-tggcagcaaattcaaactttcttcca-3′-TAMRA | 26 | 1225 | 485 |
| Reverse | 5′-tttgctgtgcttgacagatttt-3′ | 22 | 1269 | 486 |

TABLE ADB
General_screening_panel_v1.4

| | Rel. Exp. (%) Ag3541, Run | | Rel. Exp. (%) Ag3541, Run |
|---|---|---|---|
| Tissue Name | 217049294 | Tissue Name | 217049294 |
| Adipose | 0.0 | Renal ca. TK-10 | 6.0 |
| Melanoma* | 0.7 | Bladder | 3.7 |
| Hs688(A).T | | | |
| Melanoma* | 1.6 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 2.7 |
| MEL-5 | | | |
| Squamous cell | 0.0 | Colon ca.* (SW480 | 5.4 |
| carcinoma SCC-4 | | met) SW620 | |
| Testis Pool | 3.1 | Colon ca. HT29 | 0.0 |
| Prostate ca.* (bone | 1.4 | Colon ca. HCT-116 | 0.6 |
| met) PC-3 | | | |
| Prostate Pool | 2.3 | Colon ca. CaCo-2 | 0.6 |
| Placenta | 0.5 | Colon cancer tissue | 0.0 |
| Uterus Pool | 0.0 | Colon ca. SW1116 | 0.0 |
| Ovarian ca. | 2.9 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.9 | Colon Pool | 4.6 |
| OVCAR-4 | | | |
| Ovarian ca. | 27.0 | Small Intestine Pool | 6.6 |
| OVCAR-5 | | | |
| Ovarian ca. | 0.0 | Stomach Pool | 3.1 |
| IGROV-1 | | | |
| Ovarian ca. | 1.8 | Bone Marrow Pool | 1.4 |
| OVCAR-8 | | | |
| Ovary | 2.5 | Fetal Heart | 9.2 |
| Breast ca. MCF-7 | 2.4 | Heart Pool | 3.4 |
| Breast ca. MDA- | 8.0 | Lymph Node Pool | 3.9 |
| MB-231 | | | |
| Breast ca. BT 549 | 4.9 | Fetal Skeletal Muscle | 4.9 |
| Breast ca. T47D | 52.9 | Skeletal Muscle Pool | 13.5 |
| Breast ca. MDA-N | 0.0 | Spleen Pool | 0.0 |
| Breast Pool | 6.7 | Thymus pool | 4.7 |
| Trachea | 0.9 | CNS cancer | 0.9 |
| | | (glio/astro) U87-MG | |
| Lung | 1.7 | CNS cancer | 12.1 |
| | | (glio/astro) U-118-MG | |
| Fetal Lung | 2.2 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 4.2 | CNS cancer (astro) | 5.2 |
| | | SNB-75 | |
| Lung ca. NCI-H146 | 2.1 | CNS cancer (glio) | 0.0 |
| | | SNB-19 | |
| Lung ca. SHP-77 | 6.7 | CNS cancer (glio) SF- | 0.7 |
| | | 295 | |
| Lung ca. A549 | 0.0 | Brain (Amygdala) Pool | 4.2 |
| Lung ca. NCI-H526 | 0.0 | Brain (cerebellum) | 100.0 |
| Lung ca. NCI-H23 | 10.2 | Brain (fetal) | 14.7 |
| Lung ca. NCI-H460 | 3.4 | Brain (Hippocampus) | 9.0 |
| | | Pool | |
| Lung ca. HOP-62 | 0.0 | Cerebral Cortex Pool | 9.7 |
| Lung ca. NCI-H522 | 8.5 | Brain (Substantia | 3.5 |
| | | nigra) Pool | |
| Liver | 0.5 | Brain (Thalamus) Pool | 10.5 |
| Fetal Liver | 1.5 | Brain (whole) | 12.9 |
| Liver ca. HepG2 | 0.5 | Spinal Cord Pool | 7.6 |
| Kidney Pool | 9.8 | Adrenal Gland | 10.2 |
| Fetal Kidney | 7.9 | Pituitary gland Pool | 3.1 |
| Renal ca. 786-0 | 0.0 | Salivary Gland | 1.7 |
| Renal ca. A498 | 0.0 | Thyroid (female) | 0.9 |
| Renal ca. ACHN | 0.0 | Pancreatic ca. | 2.8 |
| | | CAPAN2 | |
| Renal ca. UO-31 | 3.3 | Pancreas Pool | 6.6 |

TABLE ADC
Panel 4D

| | Rel. Exp. (%) | | Rel. Exp. (%) |
|---|---|---|---|
| | Ag3541, Run | | Ag3541, Run |
| Tissue Name | 166447041 | Tissue Name | 166447041 |
| Secondary Th1 act | 2.7 | HUVEC IL-1beta | 0.0 |
| Secondary Th2 act | 4.1 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 2.1 |
| Secondary Tr1 rest | 0.0 | Lung Microvascular EC | 0.0 |
| | | none | |
| Primary Th1 act | 2.7 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 2.3 |
| lymphocyte act | | | |
| CD45RO CD4 | 1.7 | Coronery artery SMC | 0.0 |
| lymphocyte act | | TNF alpha + IL-1beta | |
| CD8 lymphocyte act | 0.0 | Astrocytes rest | 1.8 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 4.2 |
| lymphocyte act | | | |
| CD4 lymphocyte none | 0.0 | KU-812 (Basophil) | 1.4 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 2.8 | CCD1106 | 9.8 |
| | | (Keratinocytes) | |
| | | TNF alpha + IL-1beta | |
| LAK cells IL-2 | 0.0 | Liver cirrhosis | 22.2 |
| LAK cells IL-2 + IL-12 | 0.0 | Lupus kidney | 18.4 |
| LAK cells IL-2 + IFN | 0.0 | NCI-H292 none | 10.4 |
| gamma | | | |
| LAK cells IL-2 + IL-18 | 0.0 | NCI-H292 IL-4 | 7.2 |
| LAK cells | 0.0 | NCI-H292 IL-9 | 15.2 |
| PMA/ionomycin | | | |
| NK Cells IL-2 rest | 1.7 | NCI-H292 IL-13 | 3.1 |
| Two Way MLR 3 day | 0.0 | NCI-H292 IFN gamma | 7.3 |
| Two Way MLR 5 day | 5.3 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 1.7 |
| PBMC PWM | 0.0 | Lung fibroblast TNF | 5.7 |
| | | alpha + IL-1beta | |
| PBMC PHA-L | 2.0 | Lung fibroblast IL-4 | 0.0 |
| Ramos (B cell) none | 0.0 | Lung fibroblast IL-9 | 0.0 |
| Ramos (B cell) | 2.2 | Lung fibroblast IL-13 | 3.2 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 2.9 |
| and IL-4 | | CCD1070 rest | |
| EOL-1 dbcAMP | 0.0 | Dermal fibroblast | 2.9 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 3.5 | Dermal fibroblast IL-4 | 1.5 |
| Dendritic cells anti- | 0.0 | IBD Colitis 2 | 5.4 |
| CD40 | | | |
| Monocytes rest | 0.0 | IBD Crohn's | 0.0 |
| Monocytes LPS | 0.0 | Colon | 14.1 |
| Macrophages rest | 4.5 | Lung | 0.0 |
| Macrophages LPS | 2.1 | Thymus | 100.0 |
| HUVEC none | 0.0 | Kidney | 2.3 |
| HUVEC starved | 2.5 | | |

CNS_neurodegeneration_v1.0 Summary: Ag3541—Expression of this gene is low/undetectable (CTs>34.5) across all of the samples on this panel (data not shown).

General_screening_panel_v1.4 Summary: Ag3541 Significant expression of this gene is seen only in cerebellum, fetal brain, the breast cancer cell line T47D, and ovarian cancer cell line OVCAR-5 (CTs=32-35). Therefore, expression of this gene can be used to differentiate between these samples and others on this panel.

Panel 4D Summary: Ag3541—There is significant expression of this gene only in thymus (CT=33.8). Therefore, expression of this gene may be used to identify thymic tissue. Furthermore, drugs that inhibit the function of this protein may regulate T cell development in the thymus and reduce or eliminate the symptoms of T cell mediated autoimmune or inflammatory diseases, including asthma, allergies, inflammatory bowel disease, lupus erythematosus, or rheumatoid arthritis. Additionally, therapeutics designed against this putative protein may disrupt T cell development in the thymus and function as an immunosuppresant for tissue transplant.

AE. CG59309-01: Acyl-Coenzyme a Thioester Hydrolase

Expression of gene CG59309-01 was assessed using the primer-probe set Ag3540, described in Table AEA. Results of the RTQ-PCR runs are shown in Tables AEB, AEC, AED and AEE.

Table AEA. Probe Name Ag3540

TABLE AEA
Probe Name Ag3540

| | | | Start | SEQ ID |
|---|---|---|---|---|
| Primers | Sequences | Length | Position | NO: |
| Forward | 5′-ccacgttggctctagcttatta-3′ | 22 | 649 | 487 |
| Probe | TET-5′-tgaagatctccccaataacatggaca-3′-TAMRA | 26 | 677 | 488 |
| Reverse | 5′-ttcgaagtactccagggatatg-3′ | 22 | 704 | 489 |

TABLE AEB
CNS_neurodegeneration_v1.0

| | Rel. Exp. (%) Ag3540, | | Rel. Exp. (%) Ag3540, |
|---|---|---|---|
| Tissue Name | Run 210638385 | Tissue Name | Run 210638385 |
| AD 1 Hippo | 13.7 | Control (Path) 3 | 8.2 |
| | | Temporal Ctx | |
| AD 2 Hippo | 26.2 | Control (Path) 4 | 34.2 |
| | | Temporal Ctx | |
| AD 3 Hippo | 13.1 | AD 1 Occipital | 23.2 |
| | | Ctx | |
| AD 4 Hippo | 3.4 | AD 2 Occipital | 0.0 |
| | | Ctx (Missing) | |
| AD 5 hippo | 30.4 | AD 3 Occipital | 7.8 |
| | | Ctx | |
| AD 6 Hippo | 55.9 | AD 4 Occipital | 15.0 |
| | | Ctx | |
| Control 2 Hippo | 24.0 | AD 5 Occipital | 8.1 |
| | | Ctx | |
| Control 4 Hippo | 4.5 | AD 6 Occipital | 76.3 |
| | | Ctx | |
| Control (Path) 3 | 6.2 | Control 1 Occipital | 3.6 |
| Hippo | | Ctx | |
| AD 1 Temporal Ctx | 11.0 | Control 2 Occipital | 96.6 |
| | | Ctx | |
| AD 2 Temporal Ctx | 19.5 | Control 3 Occipital | 36.3 |
| | | Ctx | |
| AD 3 Temporal Ctx | 4.8 | Control 4 Occipital | 3.9 |
| | | Ctx | |
| AD 4 Temporal Ctx | 15.6 | Control (Path) 1 | 100.0 |
| | | Occipital Ctx | |
| AD 5 Inf Temporal | 36.9 | Control (Path) 2 | 7.6 |
| Ctx | | Occipital Ctx | |
| AD 5 SupTemporal | 27.4 | Control (Path) 3 | 1.6 |
| Ctx | | Occipital Ctx | |
| AD 6 Inf Temporal | 47.3 | Control (Path) 4 | 16.6 |
| Ctx | | Occipital Ctx | |
| AD 6 Sup Temporal | 64.2 | Control 1 Parietal | 8.7 |
| Ctx | | Ctx | |
| Control 1 Temporal | 7.0 | Control 2 Parietal | 20.7 |
| Ctx | | Ctx | |
| Control 2 Temporal | 53.2 | Control 3 Parietal | 27.2 |
| Ctx | | Ctx | |
| Control 3 Temporal | 19.9 | Control (Path) 1 | 88.9 |
| Ctx | | Parietal Ctx | |
| Control 4 Temporal | 10.5 | Control (Path) 2 | 10.8 |
| Ctx | | Parietal Ctx | |
| Control (Path) 1 | 68.3 | Control (Path) 3 | 10.1 |
| Temporal Ctx | | Parietal Ctx | |
| Control (Path) 2 | 25.3 | Control (Path) 4 | 47.6 |
| Temporal Ctx | | Parietal Ctx | |

TABLE AEC
General_screening_panel_v1.4

| | Rel. Exp. (%) Ag3540, Run | | Rel. Exp. (%) Ag3540, Run |
|---|---|---|---|
| Tissue Name | 217049291 | Tissue Name | 217049291 |
| Adipose | 1.3 | Renal ca. TK-10 | 0.1 |
| Melanoma* | 0.7 | Bladder | 1.1 |
| Hs688(A).T | | | |
| Melanoma* | 0.5 | Gastric ca. (liver met.) | 5.6 |
| Hs688(B).T | | NCI-N87 | |
| Melanoma* M14 | 0.2 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 10.3 |
| MEL-5 | | | |
| Squamous cell | 0.3 | Colon ca.* (SW480 | 2.8 |
| carcinoma SCC-4 | | met) SW620 | |
| Testis Pool | 0.3 | Colon ca. HT29 | 0.8 |
| Prostate ca.* (bone | 0.8 | Colon ca. HCT-116 | 0.0 |
| met) PC-3 | | | |
| Prostate Pool | 0.3 | Colon ca. CaCo-2 | 3.5 |
| Placenta | 1.4 | Colon cancer tissue | 1.4 |
| Uterus Pool | 0.1 | Colon ca. SW1116 | 0.0 |
| Ovarian ca. | 1.6 | Colon ca. Colo-205 | 3.3 |
| OVCAR-3 | | | |
| Ovarian ca. SK- | 3.6 | Colon ca. SW-48 | 1.7 |
| OV-3 | | | |
| Ovarian ca. | 0.4 | Colon Pool | 0.2 |
| OVCAR-4 | | | |
| Ovarian ca. | 23.7 | Small Intestine Pool | 0.3 |
| OVCAR-5 | | | |
| Ovarian ca. | 0.0 | Stomach Pool | 0.1 |
| IGROV-1 | | | |
| Ovarian ca. | 0.0 | Bone Marrow Pool | 0.2 |
| OVCAR-8 | | | |
| Ovary | 0.1 | Fetal Heart | 0.4 |
| Breast ca. MCF-7 | 0.0 | Heart Pool | 0.2 |
| Breast ca. MDA- | 2.5 | Lymph Node Pool | 0.3 |
| MB-231 | | | |
| Breast ca. BT 549 | 3.0 | Fetal Skeletal Muscle | 0.1 |
| Breast ca. T47D | 100.0 | Skeletal Muscle Pool | 0.4 |
| Breast ca. MDA-N | 0.0 | Spleen Pool | 0.2 |
| Breast Pool | 0.3 | Thymus Pool | 0.3 |
| Trachea | 0.4 | CNS cancer | 0.0 |
| | | (glio/astro) U87-MG | |
| Lung | 0.0 | CNS cancer | 0.3 |
| | | (glio/astro) U-118-MG | |
| Fetal Lung | 0.2 | CNS cancer | 1.0 |
| | | (neuro; met) SK-N-AS | |
| Lung ca. NCI-N417 | 0.0 | CNS cancer (astro) SF- | 0.6 |
| | | 539 | |
| Lung ca. LX-1 | 3.5 | CNS cancer (astro) | 3.1 |
| | | SNB-75 | |
| Lung ca. NCI-H146 | 0.0 | CNS cancer (glio) | 0.0 |
| | | SNB-19 | |
| Lung ca. SHP-77 | 0.1 | CNS cancer (glio) SF- | 0.2 |
| | | 295 | |
| Lung ca. A549 | 1.4 | Brain (Amygdala) Pool | 0.7 |
| Lung ca. NCI-H526 | 0.7 | Brain (cerebellum) | 2.1 |
| Lung ca. NCI-H23 | 1.3 | Brain (fetal) | 0.5 |
| Lung ca. NCI-H460 | 0.8 | Brain (Hippocampus) | 1.0 |
| | | Pool | |
| Lung ca. HOP-62 | 1.2 | Cerebral Cortex Pool | 0.9 |
| Lung ca. NCI-H522 | 0.0 | Brain (Substantia | 1.3 |
| | | nigra) Pool | |
| Liver | 2.6 | Brain (Thalamus) Pool | 1.1 |
| Fetal Liver | 0.8 | Brain (whole) | 1.4 |
| Liver ca. HepG2 | 0.1 | Spinal Cord Pool | 0.5 |
| Kidney Pool | 0.7 | Adrenal Gland | 0.8 |
| Fetal Kidney | 0.6 | Pituitary gland Pool | 0.1 |
| Renal ca. 786-0 | 0.0 | Salivary Gland | 0.2 |
| Renal ca. A498 | 0.0 | Thyroid (female) | 0.7 |
| Renal ca. ACHN | 0.0 | Pancreatic ca. | 9.4 |
| | | CAPAN2 | |
| Renal ca. UO-31 | 1.1 | Pancreas Pool | 0.9 |

TABLE AED
Panel 4D

| | Rel. Exp. (%) | | Rel. Exp. (%) |
|---|---|---|---|
| | Ag3540, Run | | Ag3540, Run |
| Tissue Name | 166447040 | Tissue Name | 166447040 |
| Secondary Th1 act | 4.8 | HUVEC IL-1beta | 1.7 |
| Secondary Th2 act | 10.2 | HUVEC IFN gamma | 0.9 |
| Secondary Tr1 act | 12.9 | HUVEC TNF alpha + | 1.5 |
| | | IFN gamma | |
| Secondary Th1 rest | 2.1 | HUVEC TNF alpha + | 0.8 |
| | | IL4 | |
| Secondary Th2 rest | 1.4 | HUVEC IL-11 | 1.5 |
| Secondary Tr1 rest | 1.6 | Lung Microvascular EC | 0.6 |
| | | none | |
| Primary Th1 act | 4.7 | Lung Microvascular EC | 0.8 |
| | | TNF alpha + IL-1beta | |
| Primary Th2 act | 6.8 | Microvascular Dermal | 1.5 |
| | | EC none | |
| Primary Tr1 act | 7.3 | Microsvasular Dermal | 0.8 |
| | | EC TNF alpha + IL-1beta | |
| Primary Th1 rest | 6.6 | Bronchial epithelium | 1.3 |
| 7 | | TNF alpha + IL1beta | |
| Primary Th2 rest | 2.6 | Small airway epithelium | 0.6 |
| | | none | |
| Primary Tr1 rest | 4.2 | Small airway epithelium | 0.0 |
| | | TNF alpha + IL-1beta | |
| CD45RA CD4 | 4.1 | Coronery artery SMC rest | 0.9 |
| lymphocyte act | | | |
| CD45RO CD4 | 10.9 | Coronery artery SMC | 0.0 |
| lymphocyte act | | TNF alpha + IL-1beta | |
| CD8 lymphocyte act | 6.6 | Astrocytes rest | 2.6 |
| Secondary CD8 | 17.0 | Astrocytes TNF alpha + | 2.1 |
| lymphocyte rest | | IL-1beta | |
| Secondary CD8 | 6.0 | KU-812 (Basophil) rest | 2.2 |
| lymphocyte act | | | |
| CD4 lymphocyte none | 2.0 | KU-812 (Basophil) | 10.2 |
| | | PMA/ionomycin | |
| 2ry Th1/Th2/Tr1_anti- | 2.4 | CCD1106 | 6.8 |
| CD95 CH11 | | (Keratinocytes) none | |
| LAK cells rest | 2.0 | CCD1106 | 25.7 |
| | | (Keratinocytes) | |
| | | TNF alpha + IL-1beta | |
| LAK cells IL-2 | 16.2 | Liver cirrhosis | 12.0 |
| LAK cells IL-2 + IL-12 | 12.8 | Lupus kidney | 5.1 |
| LAK cells IL-2 + IFN | 15.6 | NCI-H292 none | 44.8 |
| gamma | | | |
| LAK cells IL-2 + IL-18 | 7.4 | NCI-H292 IL-4 | 37.6 |
| LAK cells | 3.4 | NCI-H292 IL-9 | 41.2 |
| PMA/ionomycin | | | |
| NK Cells IL-2 rest | 9.0 | NCI-H292 IL-13 | 19.8 |
| Two Way MLR 3 day | 10.5 | NCI-H292 IFN gamma | 30.1 |
| Two Way MLR 5 day | 7.2 | HPAEC none | 1.2 |
| Two Way MLR 7 day | 8.9 | HPAEC TNF alpha + IL- | 3.3 |
| | | 1beta | |
| PBMC rest | 0.5 | Lung fibroblast none | 0.9 |
| PBMC PWM | 3.8 | Lung fibroblast TNF | 0.7 |
| | | alpha + IL-1beta | |
| PBMC PHA-L | 1.0 | Lung fibroblast IL-4 | 0.5 |
| Ramos (B cell) none | 0.0 | Lung fibroblast IL-9 | 0.0 |
| Ramos (B cell) | 0.0 | Lung fibroblast IL-13 | 0.9 |
| ionomycin | | | |
| B lymphocytes PWM | 10.3 | Lung fibroblast IFN | 1.2 |
| | | gamma | |
| B lymphocytes CD40L | 3.8 | Dermal fibroblast | 1.1 |
| and IL-4 | | CCD1070 rest | |
| EOL-1 dbcAMP | 0.0 | Dermal fibroblast | 18.9 |
| | | CCD1070 TNF alpha | |
| EOL-1 dbcAMP | 0.0 | Dermal fibroblast | 1.9 |
| PMA/ionomycin | | CCD1070 IL-1beta | |
| Dendritic cells none | 14.9 | Dermal fibroblast IFN | 0.0 |
| | | gamma | |
| Dendritic cells LPS | 8.9 | Dermal fibroblast IL-4 | 1.5 |
| Dendritic cells anti- | 7.9 | IBD Colitis 2 | 2.9 |
| CD40 | | | |
| Monocytes rest | 0.0 | IBD Crohn's | 1.9 |
| Monocytes LPS | 0.6 | Colon | 82.9 |
| Macrophages rest | 40.3 | Lung | 9.7 |
| Macrophages LPS | 6.1 | Thymus | 100.0 |
| HUVEC none | 1.1 | Kidney | 1.8 |
| HUVEC starved | 1.4 | | |

TABLE AEE
Panel 5 Islet

| | Rel. Exp. (%) | | Rel. Exp. (%) |
|---|---|---|---|
| | Ag3540, Run | | Ag3540, Run |
| Tissue Name | 242386396 | Tissue Name | 242386396 |
| 97457_Patient- | 3.3 | 94709_Donor 2 AM - A_adipose | 9.1 |
| 02go_adipose | | | |
| 97476_Patient- | 0.8 | 94710_Donor 2 AM - B_adipose | 1.6 |
| 07sk_skeletal muscle | | | |
| 97477_Patient- | 0.0 | 94711_Donor 2 AM - C_adipose | 1.4 |
| 07ut_uterus | | | |
| 97478_Patient- | 12.9 | 94712_Donor 2 AD - A_adipose | 2.8 |
| 07pl_placenta | | | |
| 99167_Bayer Patient 1 | 15.5 | 94713_Donor 2 AD - B_adipose | 5.8 |
| 97482_Patient- | 3.4 | 94714_Donor 2 AD - C_adipose | 4.2 |
| 08ut_uterus | | | |
| 97483_Patient- | 3.4 | 94742_Donor 3 U - | 3.0 |
| 08pl_placenta | | A_Mesenchymal Stem Cells | |
| 97486_Patient- | 100.0 | 94743_Donor 3 U - | 1.1 |
| 09sk_skeletal muscle | | B_Mesenchymal Stem Cells | |
| 97487_Patient- | 1.6 | 94730_Donor 3 AM - A_adipose | 4.3 |
| 09ut_uterus | | | |
| 97488_Patient- | 2.6 | 94731_Donor 3 AM - B_adipose | 2.0 |
| 09pl_placenta | | | |
| 97492_Patient- | 3.1 | 94732_Donor 3 AM - C_adipose | 2.0 |
| 10ut_uterus | | | |
| 97493_Patient- | 23.2 | 94733_Donor 3 AD - A_adipose | 10.7 |
| 10pl_placenta | | | |
| 97495_Patient- | 0.8 | 94734_Donor 3 AD - B_adipose | 3.0 |
| 11go_adipose | | | |
| 97496_Patient- | 0.0 | 94735_Donor 3 AD - C_adipose | 4.0 |
| 11sk_skeletal muscle | | | |
| 97497_Patient- | 2.5 | 77138_Liver_HepG2untreated | 0.7 |
| 11ut_uterus | | | |
| 97498_Patient- | 6.7 | 73556_Heart_Cardiac stromal | 0.0 |
| 11pl_placenta | | cells (primary) | |
| 97500_Patient- | 6.5 | 81735_Small Intestine | 4.8 |
| 12go_adipose | | | |
| 97501_Patient- | 4.5 | 72409_Kidney_Proximal | 0.7 |
| 12sk_skeletal muscle | | Convoluted Tubule | |
| 97502_Patient- | 6.7 | 82685_Small | 3.6 |
| 12ut_uterus | | intestine_Duodenum | |
| 97503_Patient- | 2.4 | 90650_Adrenal_Adrenocortical | 0.6 |
| 12pl_placenta | | adenoma | |
| 94721_Donor 2 U - | 2.2 | 72410_Kidney_HRCE | 8.0 |
| A_Mesenchymal | | | |
| Stem Cells | | | |
| 94722_Donor 2 U - | 0.6 | 72411_Kidney_HRE | 8.5 |
| B_Mesenchymal | | | |
| Stem Cells | | | |
| 94723_Donor 2 U - | 3.1 | 73139_Uterus_Uterine smooth | 0.0 |
| C_Mesenchymal | | muscle cells | |
| Stem Cells | | | |

CNS_neurodegeneration_v1.0 Summary: Ag3540—This panel confirms the expression of this gene at low levels in the brain in an independent group of individuals. However, no differential expression of this gene was detected between Alzheimer's diseased postmortem brains and those of non-demented controls in this experiment.

General_screening_panel_v1.4 Summary: Ag3540 This gene is most highly expressed in a breast cancer cell line (CT=27.1). Thus, expression of this gene could be used to differentiate between this sample and other samples on this panel and as a marker to detect the presence of breast cancer. Furthermore, therapeutic modulation of the expression or function of this gene may be effective in the treatment of breast cancer.

Among metabolic tissues, this gene, an acyl coA thioesterase homolog, has a low level of expression in adipose, adult and fetal liver, adrenal, thyroid and pancreas. Acyl CoA thioesterases have multiple roles in lipid homeostasis. Therefore, therapeutic modulation of this gene product may be a treatment for endocrine and metabolic disease, including Types 1 and 2 diabetes and obesity.

In addition, this gene is expressed in all CNS regions examined. Thus, therapeutic modulation of the expression or function of this gene may be effective in the treatment of neurologic disorders such as Alzheimer's disease, Parkinson's disease, epilepsy, stroke, schizophrenia and multiple sclerosis.

### REFERENCES

1. Hunt M C, Alexson S E. The role Acyl-CoA thioesterases play in mediating intracellular lipid metabolism. Prog Lipid Res. March 2002;41(2):99-130.

2. Hunt M C, Nousiainen S E, Huttunen M K, Orii K E, Svensson L T, Alexson S E. Peroxisome proliferator-induced long chain acyl-CoA thioesterases comprise a highly conserved novel multi-gene family involved in lipid metabolism. J. Biol. Chem. Nov. 26, 1999;274(48):34317-26.

Panel 4D Summary: Ag3540 Highest expression of the CG59309-01 gene is seen in the thymus and colon (CTs=31.5). Significant levels of expression are also seen in a cluter of treated and untreated samples derived from the NCI-H292 mucoepidermoid cell line. Thus, expression of this gene could be used as a marker for thymus and colon. Furthermore, therapeutic modulation of the expression or function of this gene may regulate T cell development in the thymus and reduce or eliminate the symptoms of T cell mediated autoimmune or inflammatory diseases, including asthma, allergies, inflammatory bowel disease, lupus erythematosus, or rheumatoid arthritis. Additionally, small molecule or antibody therapeutics designed against this putative protein may disrupt T cell development in the thymus and function as an immunosuppresant for tissue transplant.

Panel 5 Islet Summary: Ag3540 This gene has moderate expression in skeletal muscle, (highest expression CT=30.5). Acyl CoA thioesterases function in peroxisomal fatty acid oxidation. Therefore, therapeutic modulation of this homolog may increase fatty acid oxidation in muscle and be a treatment for Type 2 diabetes and obesity.

REFERENCES

1. Hunt M C, Solaas K, Kase B F, Alexson S E. Characterization of an acyl-coA thioesteirase that functions as a major regulator of peroxisomal lipid metabolism. J. Biol. Chem. Jan. 11, 2002;277(2):1128-38.

AF. CG57364-01: CG6896

Expression of gene CG57364-01 was assessed using the primer-probe sets Ag3218 and Ag3378, described in Tables AFA and AFB. Results of the RTQ-PCR runs are shown in Tables AFC, AFD, AFE and AFF.

Table AFA. Probe Name Ag3218

TABLE AFA
Probe Name Ag3218

| | | | Start | SEQ ID |
|---|---|---|---|---|
| Primers | Sequences | Length | Position | NO: |
| Forward | 5′-ctcctgaagcaggtcctctt-3′ | 20 | 249 | 490 |
| Probe | TET-5′-cctcccagtgttgtccttctggagg-3′-TAMRA | 25 | 270 | 491 |
| Reverse | 5′-gacttcttccaggtcatttcg-3′ | 21 | 303 | 492 |

Table AFB. Probe Name Ag3378

TABLE AFB
Probe Name Ag3378

| | | | Start | SEQ ID |
|---|---|---|---|---|
| Primers | Sequences | Length | Position | NO: |
| Forward | 5′-ctcctgaagcaggtcctctt-3′ | 20 | 249 | 493 |
| Probe | TET-5′-cctcccagtgttgtccttctggagg-3′-TAMRA | 25 | 270 | 494 |
| Reverse | 5′-gacttcttccaggtcatttcg-3′ | 21 | 303 | 495 |

TABLE AFC
CNS_neurodegeneration_v1.z0

| | Rel. Exp. (%) | Rel. Exp. (%) | | Rel. Exp. (%) | Rel. Exp. (%) |
|---|---|---|---|---|---|
| | Ag3218, Run | Ag3378, Run | Tissue | Ag3218, Run | Ag3378, Run |
| Tissue Name | 209861784 | 210154573 | Name | 209861784 | 210154573 |
| AD 1 Hippo | 37.6 | 30.4 | Control | 17.6 | 16.7 |
| | | | (Path) 3 | | |
| | | | Temporal | | |
| | | | Ctx | | |
| AD 2 Hippo | 31.0 | 37.6 | Control | 37.6 | 31.2 |
| | | | (Path) 4 | | |
| | | | Temporal | | |
| | | | Ctx | | |
| AD 3 Hippo | 34.2 | 21.5 | AD 1 | 56.3 | 40.3 |
| | | | Occipital | | |
| | | | Ctx | | |
| AD 4 Hippo | 40.6 | 25.3 | AD 2 | 0.0 | 0.0 |
| | | | Occipital | | |
| | | | Ctx | | |
| | | | (Missing) | | |
| AD 5 hippo | 100.0 | 69.3 | AD 3 | 43.2 | 24.1 |
| | | | Occipital | | |
| | | | Ctx | | |
| AD 6 Hippo | 62.9 | 55.9 | AD 4 | 80.1 | 24.3 |
| | | | Occipital | | |
| | | | Ctx | | |
| Control 2 | 55.1 | 52.9 | AD 5 | 17.9 | 25.2 |
| Hippo | | | Occipital | | |
| | | | Ctx | | |
| Control 4 | 35.4 | 39.5 | AD 6 | 66.9 | 55.5 |
| Hippo | | | Occipital | | |
| | | | Ctx | | |
| Control (Path) | 22.8 | 26.8 | Control 1 | 27.9 | 17.4 |
| 3 Hippo | | | Occipital | | |
| | | | Ctx | | |
| AD 1 Temporal | 40.3 | 28.3 | Control 2 | 94.0 | 64.6 |
| Ctx | | | Occipital | | |
| | | | Ctx | | |
| AD 2 Temporal | 83.5 | 94.6 | Control 3 | 43.5 | 40.6 |
| Ctx | | | Occipital | | |
| | | | Ctx | | |
| AD 3 Temporal | 30.8 | 24.5 | Control 4 | 20.3 | 22.5 |
| Ctx | | | Occipital | | |
| | | | Ctx | | |
| AD 4 Temporal | 61.1 | 26.8 | Control | 79.6 | 51.4 |
| Ctx | | | (Path) 1 | | |
| | | | Occipital | | |
| | | | Ctx | | |
| AD 5 Inf | 84.7 | 100.0 | Control | 34.4 | 24.7 |
| Temporal Ctx | | | (Path) 2 | | |
| | | | Occipital | | |
| | | | Ctx | | |
| AD 5 | 55.9 | 39.8 | Control | 25.2 | 16.2 |
| SupTemporal | | | (Path) 3 | | |
| Ctx | | | Occipital | | |
| | | | Ctx | | |
| AD 6 Inf | 47.0 | 46.0 | Control | 76.3 | 45.1 |
| Temporal Ctx | | | (Path) 4 | | |
| | | | Occipital | | |
| | | | Ctx | | |
| AD 6 Sup | 63.7 | 41.2 | Control 1 | 31.0 | 21.9 |
| Temporal Ctx | | | Parietal Ctx | | |
| Control 1 | 32.8 | 18.0 | Control 2 | 67.4 | 45.1 |
| Temporal Ctx | | | Parietal Ctx | | |
| Control 2 | 52.1 | 39.2 | Control 3 | 31.4 | 29.3 |
| Temporal Ctx | | | Parietal Ctx | | |
| Control 3 | 34.9 | 28.1 | Control | 48.6 | 58.6 |
| Temporal Ctx | | | (Path) 1 | | |
| | | | Parietal Ctx | | |
| Control 4 | 62.9 | 36.3 | Control | 46.3 | 27.0 |
| Temporal Ctx | | | (Path) 2 | | |
| | | | Parietal Ctx | | |
| Control (Path) | 75.8 | 50.0 | Control | 26.1 | 23.8 |
| 1 Temporal Ctx | | | (Path) 3 | | |
| | | | Parietal Ctx | | |
| Control (Path) | 56.6 | 41.8 | Control | 48.0 | 54.3 |
| 2 Temporal Ctx | | | (Path) 4 | | |
| | | | Parietal Ctx | | |

TABLE AFD
Panel 1.3D

| | Rel. | Rel. | | Rel. | Rel. |
|---|---|---|---|---|---|
| | Exp. (%) | Exp. (%) | | Exp. (%) | Exp. (%) |
| | Ag3218, | Ag3378, | | Ag3218, | Ag3378, |
| | Run | Run | | Run | Run |
| Tissue Name | 168013878 | 165674263 | Tissue Name | 168013878 | 165674263 |
| Liver | 10.7 | 20.2 | Kidney (fetal) | 48.3 | 13.9 |
| adenocarcinoma | | | | | |
| Pancreas | 10.8 | 13.1 | Renal ca. 786-0 | 15.6 | 10.4 |
| Pancreatic ca. | 9.6 | 5.4 | Renal ca. | 19.2 | 14.9 |
| CAPAN 2 | | | A498 | | |
| Adrenal gland | 5.1 | 18.4 | Renal ca. RXF | 39.0 | 33.2 |
| | | | 393 | | |
| Thyroid | 12.3 | 33.2 | Renal ca. | 12.1 | 11.3 |
| | | | ACHN | | |
| Salivary gland | 5.1 | 5.5 | Renal ca. UO- | 18.9 | 17.8 |
| | | | 31 | | |
| Pituitary gland | 21.5 | 74.7 | Renal ca. TK- | 20.0 | 10.1 |
| | | | 10 | | |
| Brain (fetal) | 19.5 | 36.1 | Liver | 18.0 | 8.7 |
| Brain (whole) | 22.1 | 29.9 | Liver (fetal) | 5.5 | 25.3 |
| Brain (amygdala) | 57.4 | 46.7 | Liver ca. | 14.2 | 14.1 |
| | | | (hepatoblast) | | |
| | | | HepG2 | | |
| Brain (cerebellum) | 25.2 | 23.5 | Lung | 14.1 | 18.7 |
| Brain | 28.1 | 85.9 | Lung (fetal) | 17.2 | 4.0 |
| (hippocampus) | | | | | |
| Brain (substantia | 11.5 | 16.7 | Lung ca. | 6.5 | 14.8 |
| nigra) | | | (small cell) | | |
| | | | LX-1 | | |
| Brain (thalamus) | 57.0 | 67.4 | Lung ca. | 20.6 | 4.8 |
| | | | (small cell) | | |
| | | | NCI-H69 | | |
| Cerebral Cortex | 75.8 | 36.9 | Lung ca. | 100.0 | 39.8 |
| | | | (s.cell var.) | | |
| | | | SHP-77 | | |
| Spinal cord | 9.7 | 13.2 | Lung ca. | 5.0 | 37.1 |
| | | | (large | | |
| | | | cell)NCI- | | |
| | | | H460 | | |
| glio/astro U87- | 22.8 | 13.6 | Lung ca. (non- | 27.7 | 13.6 |
| MG | | | sm. cell) A549 | | |
| glio/astro U-118- | 37.4 | 79.6 | Lung ca. (non- | 61.1 | 44.1 |
| MG | | | s.cell) NCI- | | |
| | | | H23 | | |
| astrocytoma | 29.9 | 14.9 | Lung ca. (non- | 29.9 | 13.7 |
| SW1783 | | | s.cell) HOP- | | |
| | | | 62 | | |
| neuro*; met SK- | 17.1 | 52.5 | Lung ca. (non- | 11.3 | 3.1 |
| N-AS | | | s.cl) NCI- | | |
| | | | H522 | | |
| astrocytoma SF- | 15.5 | 16.0 | Lung ca. | 23.2 | 13.5 |
| 539 | | | (squam.) SW | | |
| | | | 900 | | |
| astrocytoma SNB- | 43.8 | 50.0 | Lung ca. | 41.5 | 10.2 |
| 75 | | | (squam.) NCI- | | |
| | | | H596 | | |
| glioma SNB-19 | 17.9 | 26.2 | Mammary | 14.8 | 35.1 |
| | | | gland | | |
| glioma U251 | 47.6 | 39.0 | Breast ca.* | 48.6 | 39.0 |
| | | | (pl.ef) MCF-7 | | |
| glioma SF-295 | 12.3 | 10.7 | Breast ca.* | 25.9 | 60.7 |
| | | | (pl.ef) MDA- | | |
| | | | MB-231 | | |
| Heart (fetal) | 38.4 | 8.0 | Breast ca.* | 77.4 | 21.2 |
| | | | (pl.ef) T47D | | |
| Heart | 3.5 | 5.0 | Breast ca. BT- | 47.0 | 95.9 |
| | | | 549 | | |
| Skeletal muscle | 17.0 | 10.0 | Breast ca. | 16.6 | 7.3 |
| (fetal) | | | MDA-N | | |
| Skeletal muscle | 4.4 | 7.2 | Ovary | 10.1 | 4.7 |
| Bone marrow | 1.3 | 14.7 | Ovarian ca. | 36.3 | 31.2 |
| | | | OVCAR-3 | | |
| Thymus | 13.9 | 12.3 | Ovarian ca. | 33.0 | 20.7 |
| | | | OVCAR-4 | | |
| Spleen | 2.6 | 12.9 | Ovarian ca. | 42.6 | 15.7 |
| | | | OVCAR-5 | | |
| Lymph node | 1.7 | 15.9 | Ovarian ca. | 8.7 | 5.2 |
| | | | OVCAR-8 | | |
| Colorectal | 18.2 | 11.8 | Ovarian ca. | 11.3 | 15.1 |
| | | | IGROV-1 | | |
| Stomach | 14.8 | 33.7 | Ovarian ca.* | 43.5 | 17.0 |
| | | | (ascites) SK- | | |
| | | | OV-3 | | |
| Small intestine | 18.3 | 66.0 | Uterus | 10.5 | 21.8 |
| Colon ca. SW480 | 12.9 | 14.2 | Placenta | 2.6 | 15.0 |
| Colon ca.* | 17.0 | 14.2 | Prostate | 11.7 | 30.6 |
| SW620 (SW480 | | | | | |
| met) | | | | | |
| Colon ca. HT29 | 17.2 | 18.8 | Prostate ca.* | 35.4 | 40.3 |
| | | | (bone met) PC-3 | | |
| Colon ca. HCT- | 16.5 | 18.2 | Testis | 23.3 | 100.0 |
| 116 | | | | | |
| Colon ca. CaCo-2 | 20.9 | 7.4 | Melanoma | 5.0 | 1.4 |
| | | | Hs688(A).T | | |
| Colon ca. | 14.7 | 21.9 | Melanoma* | 6.0 | 3.5 |
| tissue (ODO3866) | | | (met) | | |
| | | | Hs688(B).T | | |
| Colon ca. HCC- | 22.1 | 13.1 | Melanoma | 14.3 | 12.2 |
| 2998 | | | UACC-62 | | |
| Gastric ca.* (liver | 48.6 | 82.4 | Melanoma | 3.1 | 8.2 |
| met) NCI-N87 | | | M14 | | |
| Bladder | 6.2 | 4.7 | Melanoma | 30.1 | 8.4 |
| | | | LOX IMVI | | |
| Trachea | 12.8 | 49.3 | Melanoma* | 21.8 | 13.1 |
| | | | (met) SK- | | |
| | | | MEL-5 | | |
| Kidney | 43.5 | 35.4 | Adipose | 9.2 | 3.0 |

TABLE AFE
Panel 2.2

| | Rel. Exp. (%) | | Rel. Exp. (%) |
|---|---|---|---|
| | Ag3218, Run | | Ag3218, Run |
| Tissue Name | 174416494 | Tissue Name | 174416494 |
| Normal Colon | 5.9 | Kidney Margin | 70.2 |
| | | (OD04348) | |
| Colon cancer | 5.6 | Kidney malignant | 3.9 |
| (OD06064) | | cancer (OD06204B) | |
| Colon Margin | 3.6 | Kidney normal | 6.7 |
| (OD06064) | | adjacent tissue | |
| | | (OD06204E) | |
| Colon cancer | 6.3 | Kidney Cancer | 15.1 |
| (OD06159) | | (OD04450-01) | |
| Colon Margin | 7.0 | Kidney Margin | 3.1 |
| (OD06159) | | (OD04450-03) | |
| Colon cancer | 2.6 | Kidney Cancer | 2.5 |
| (OD06297-04) | | 8120613 | |
| Colon Margin | 5.6 | Kidney Margin | 18.2 |
| (OD06297-05) | | 8120614 | |
| CC Gr.2 ascend colon | 20.0 | Kidney Cancer | 2.4 |
| (ODO3921) | | 9010320 | |
| CC Margin (ODO3921) | 13.7 | Kidney Margin | 4.4 |
| | | 9010321 | |
| Colon cancer metastasis | 0.0 | Kidney Cancer | 23.0 |
| (OD06104) | | 8120607 | |
| Lung Margin | 11.0 | Kidney Margin | 15.1 |
| (OD06104) | | 8120608 | |
| Colon mets to lung | 29.9 | Normal Uterus | 2.3 |
| (OD04451-01) | | | |
| Lung Margin | 0.3 | Uterine Cancer 064011 | 6.1 |
| (OD04451-02) | | | |
| Normal Prostate | 5.6 | Normal Thyroid | 6.6 |
| Prostate Cancer | 3.9 | Thyroid Cancer | 6.8 |
| (OD04410) | | 064010 | |
| Prostate Margin | 6.1 | Thyroid Cancer | 11.9 |
| (OD04410) | | A302152 | |
| Normal Ovary | 7.0 | Thyroid Margin | 7.7 |
| | | A302153 | |
| Ovarian cancer | 1.3 | Normal Breast | 3.4 |
| (OD06283-03) | | | |
| Ovarian Margin | 0.0 | Breast Cancer | 9.9 |
| (OD06283-07) | | (OD04566) | |
| Ovarian Cancer 064008 | 31.2 | Breast Cancer 1024 | 16.8 |
| Ovarian cancer | 3.5 | Breast Cancer | 100.0 |
| (OD06145) | | (OD04590-01) | |
| Ovarian Margin | 8.4 | Breast Cancer Mets | 26.2 |
| (OD06145) | | (OD04590-03) | |
| Ovarian cancer | 13.7 | Breast Cancer | 36.3 |
| (OD06455-03) | | Metastasis (OD04655- | |
| | | 05) | |
| Ovarian Margin | 1.1 | Breast Cancer 064006 | 5.4 |
| (OD06455-07) | | | |
| Normal Lung | 5.4 | Breast Cancer 9100266 | 12.8 |
| Invasive poor diff. lung | 14.5 | Breast Margin | 1.0 |
| adeno (ODO4945-01) | | 9100265 | |
| Lung Margin | 2.7 | Breast Cancer | 3.3 |
| (ODO4945-03) | | A209073 | |
| Lung Malignant Cancer | 1.8 | Breast Margin | 11.7 |
| (OD03126) | | A2090734 | |
| Lung Margin | 5.1 | Breast cancer | 6.9 |
| (OD03126) | | (OD06083) | |
| Lung Cancer | 12.8 | Breast cancer node | 10.7 |
| (OD05014A) | | metastasis (OD06083) | |
| Lung Margin | 3.3 | Normal Liver | 9.4 |
| (OD05014B) | | | |
| Lung cancer | 6.3 | Liver Cancer 1026 | 2.6 |
| (OD06081) | | | |
| Lung Margin | 2.7 | Liver Cancer 1025 | 9.7 |
| (OD06081) | | | |
| Lung Cancer | 12.9 | Liver Cancer 6004-T | 10.4 |
| (OD04237-01) | | | |
| Lung Margin | 6.4 | Liver Tissue 6004-N | 5.3 |
| (OD04237-02) | | | |
| Ocular Melanoma | 4.6 | Liver Cancer 6005-T | 4.2 |
| Metastasis | | | |
| Ocular Melanoma | 0.1 | Liver Tissue 6005-N | 11.5 |
| Margin (Liver) | | | |
| Melanoma Metastasis | 1.6 | Liver Cancer 064003 | 22.5 |
| Melanoma Margin | 4.6 | Normal Bladder | 6.1 |
| (Lung) | | | |
| Normal Kidney | 10.4 | Bladder Cancer 1023 | 10.8 |
| Kidney Ca, Nuclear | 14.6 | Bladder Cancer | 15.1 |
| grade 2 (OD04338) | | A302173 | |
| Kidney Margin | 10.5 | Normal Stomach | 15.0 |
| (OD04338) | | | |
| Kidney Ca Nuclear | 44.8 | Gastric Cancer | 7.1 |
| grade 1/2 (OD04339) | | 9060397 | |
| Kidney Margin | 17.7 | Stomach Margin | 10.4 |
| (OD04339) | | 9060396 | |
| Kidney Ca, Clear cell | 5.3 | Gastric Cancer | 8.4 |
| type (OD04340) | | 9060395 | |
| Kidney Margin | 25.3 | Stomach Margin | 10.4 |
| (OD04340) | | 9060394 | |
| Kidney Ca, Nuclear | 7.5 | Gastric Cancer 064005 | 7.7 |
| grade 3 (OD04348) | | | |

TABLE AFF
Panel 4D

| | Rel. | Rel. | | Rel. | Rel. |
|---|---|---|---|---|---|
| | Exp. (%) | Exp. (%) | | Exp. (%) | Exp. (%) |
| | Ag3218, | Ag3378, | | Ag3218, | Ag3378, |
| | Run | Run | | Run | Run |
| Tissue Name | 164682519 | 165296553 | Tissue Name | 164682519 | 165296553 |
| Secondary Th1 act | 18.2 | 25.7 | HUVEC IL-1beta | 14.5 | 12.9 |
| Secondary Th2 act | 39.0 | 26.6 | HUVEC IFN | 47.0 | 25.3 |
| | | | gamma | | |
| Secondary Tr1 act | 33.2 | 19.1 | HUVEC TNF | 43.5 | 45.1 |
| | | | alpha + IFN | | |
| | | | gamma | | |
| Secondary Th1 rest | 9.5 | 12.2 | HUVEC TNF | 37.1 | 48.0 |
| | | | alpha + IL4 | | |
| Secondary Th2 rest | 11.2 | 5.1 | HUVEC IL-11 | 43.5 | 18.0 |
| Secondary Tr1 rest | 22.7 | 8.0 | Lung | 16.8 | 61.6 |
| | | | Microvascular EC | | |
| | | | none | | |
| Primary Th1 act | 43.2 | 27.9 | Lung | 18.6 | 14.7 |
| | | | Microvascular EC | | |
| | | | TNF alpha + IL- | | |
| | | | 1beta | | |
| Primary Th2 act | 30.1 | 12.0 | Microvascular | 31.2 | 23.8 |
| | | | Dermal EC none | | |
| Primary Tr1 act | 24.7 | 14.4 | Microsvasular | 66.4 | 22.1 |
| | | | Dermal EC | | |
| | | | TNF alpha + IL- | | |
| | | | 1beta | | |
| Primary Th1 rest | 25.2 | 17.4 | Bronchial | 30.6 | 29.3 |
| | | | epithelium | | |
| | | | TNF alpha + | | |
| | | | IL-1beta | | |
| Primary Th2 rest | 15.5 | 7.5 | Small airway | 36.1 | 24.3 |
| | | | epithelium none | | |
| Primary Tr1 rest | 21.3 | 6.7 | Small airway | 76.3 | 62.9 |
| | | | epithelium | | |
| | | | TNF alpha + IL- | | |
| | | | 1beta | | |
| CD45RA CD4 | 35.4 | 16.6 | Coronery artery | 49.7 | 28.1 |
| lymphocyte act | | | SMC rest | | |
| CD45RO CD4 | 27.9 | 25.9 | Coronery artery | 25.3 | 23.7 |
| lymphocyte act | | | SMC TNF | | |
| | | | alpha + IL-1beta | | |
| CD8 lymphocyte | 21.0 | 14.8 | Astrocytes rest | 22.2 | 31.2 |
| act | | | | | |
| Secondary CD8 | 39.2 | 17.8 | Astrocytes | 26.1 | 25.0 |
| lymphocyte rest | | | TNF alpha + IL- | | |
| | | | 1beta | | |
| Secondary CD8 | 20.9 | 7.4 | KU-812 | 90.8 | 85.3 |
| lymphocyte act | | | (Basophil) rest | | |
| CD4 lymphocyte | 4.5 | 11.8 | KU-812 | 87.1 | 72.2 |
| none | | | (Basophil) | | |
| | | | PMA/ionomycin | | |
| 2ry | 2.6 | 10.0 | CCD1106 | 36.6 | 36.9 |
| Th1/Th2/Tr1_anti- | | | (Keratinocytes) | | |
| CD95 CH11 | | | none | | |
| LAK cells rest | 11.7 | 12.3 | CCD1106 | 33.4 | 20.4 |
| | | | (Keratinocytes) | | |
| | | | TNF alpha + IL- | | |
| | | | 1beta | | |
| LAK cells IL-2 | 6.8 | 27.5 | Liver cirrhosis | 25.5 | 19.9 |
| LAK cells IL- | 37.1 | 11.6 | Lupus kidney | 44.4 | 15.7 |
| 2 + IL-12 | | | | | |
| LAK cells IL- | 20.7 | 19.1 | NCI-H292 none | 79.6 | 64.6 |
| 2 + IFN gamma | | | | | |
| LAK cells IL-2 + | 21.9 | 14.7 | NCI-H292 IL-4 | 85.3 | 96.6 |
| IL-18 | | | | | |
| LAK cells | 4.7 | 3.3 | NCI-H292 IL-9 | 100.0 | 98.6 |
| PMA/ionomycin | | | | | |
| NK Cells IL-2 rest | 11.9 | 11.3 | NCI-H292 IL-13 | 68.8 | 50.7 |
| Two Way MLR 3 | 23.7 | 11.0 | NCI-H292 IFN | 80.1 | 56.6 |
| day | | | gamma | | |
| Two Way MLR 5 | 12.5 | 6.1 | HPAEC none | 38.4 | 27.2 |
| day | | | | | |
| Two Way MLR 7 | 12.3 | 8.7 | HPAEC TNF | 42.6 | 43.2 |
| day | | | alpha + IL-1beta | | |
| PBMC rest | 6.0 | 5.7 | Lung fibroblast | 31.2 | 21.3 |
| | | | none | | |
| PBMC PWM | 40.3 | 27.7 | Lung fibroblast | 14.7 | 24.5 |
| | | | TNF alpha + IL- | | |
| | | | 1beta | | |
| PBMC PHA-L | 37.9 | 17.7 | Lung fibroblast | 47.0 | 42.6 |
| | | | IL-4 | | |
| Ramos (B cell) | 11.7 | 14.9 | Lung fibroblast | 49.3 | 30.6 |
| none | | | IL-9 | | |
| Ramos (B cell) | 33.9 | 26.8 | Lung fibroblast | 36.6 | 42.6 |
| ionomycin | | | IL-13 | | |
| B lymphocytes | 33.7 | 40.9 | Lung fibroblast | 44.8 | 22.5 |
| PWM | | | IFN gamma | | |
| B lymphocytes | 34.4 | 18.3 | Dermal fibroblast | 33.7 | 47.3 |
| CD40L and IL-4 | | | CCD1070 rest | | |
| EOL-1 dbcAMP | 50.0 | 28.1 | Dermal fibroblast | 47.3 | 33.2 |
| | | | CCD1070 TNF | | |
| | | | alpha | | |
| EOL-1 dbcAMP | 44.1 | 32.1 | Dermal fibroblast | 50.0 | 34.6 |
| PMA/ionomycin | | | CCD1070 IL- | | |
| | | | 1beta | | |
| Dendritic cells | 33.9 | 19.6 | Dermal fibroblast | 24.0 | 34.4 |
| none | | | IFN gamma | | |
| Dendritic cells LPS | 21.9 | 10.2 | Dermal fibroblast | 24.3 | 32.8 |
| | | | IL-4 | | |
| Dendritic cells | 49.7 | 33.9 | IBD Colitis 2 | 6.0 | 11.7 |
| anti-CD40 | | | | | |
| Monocytes rest | 10.7 | 10.3 | IBD Crohn's | 25.3 | 26.1 |
| Monocytes LPS | 30.6 | 9.3 | Colon | 70.7 | 100.0 |
| Macrophages rest | 41.2 | 33.7 | Lung | 64.6 | 17.7 |
| Macrophages LPS | 20.0 | 7.5 | Thymus | 80.7 | 56.3 |
| HUVEC none | 26.8 | 29.5 | Kidney | 26.4 | 41.5 |
| HUVEC starved | 26.2 | 37.6 | | | |

CNS_neurodegeneration_v1.0 Summary: Ag3218/Ag3378—Two different experiments using probe/primer sets with the same sequence are in very good agreement. This panel confirms the expression of this gene at low levels to moderate levels in the brain in an independent group of individuals. However, no differential expression of this gene was detected, between Alzheimer's diseased postmortem brains and those of non-demented controls in this experiment. Please see Panel 1.3D for a discussion of the potential utility of this gene in treatment of central nervous system disorders.

Panel 1.3D Summary: Ag3218/Ag3378—Two different experiments using probe/primer sets with the same sequence are in good agreement. Highest expression is seen in testis and a lung cancer cell line (CTs=30-31). This panel confirms the expression of this gene at low levels in the brain. Therefore, this gene may play a role in central nervous system disorders such as Alzheimer's disease, Parkinson's disease, epilepsy, multiple sclerosis, schizophrenia and depression.

This gene product is also expressed in adipose, pancreas, thyroid, pituitary, heart, and liver. This widespread expression in tissues with metabolic function suggests that this gene product may be important for the pathogenesis, diagnosis, and/or treatment of metabolic and endocrine diseases, including obesity and Types 1 and 2 diabetes.

Based on expression in this panel, this gene may be involved in gastric, pancreatic, brain, colon, renal, lung, breast, ovarian and prostate cancer as well as melanomas. Thus, expression of this gene could be used as a diagnostic marker for the presence of these cancers. Furthermore, therapeutic inhibition using antibodies or small molecule drugs might be of use in the treatment of these cancers.

Panel 2.2 Summary: Ag3218—This gene is expressed at low to moderate levels in many samples on this panel, with the highest levels of expression in breast cancer sample OD04590-01 (CT=30.3). This gene is expressed in a cluster of breast cancer samples with no expression in normal breast (CTh35). Similarly, this gene is expressed in ovarian cancer samples at higher levels than the matched margin samples.

Interestingly, this gene is expressed at higher levels in kidney cancer margin samples than in the matched cancer samples.

This gene is homologous to a mouse myosin phosphatase targeting subunit (MYPT) which have been found to play a role in cell division. MYPT undergoes mitosis-specific phosphorylation which is reversed during cytokinesis.

### REFERENCES

1. Totsukawa G, Yamakita Y, Yamashiro S, Hosoya H, Hartshorne D J, Matsumura F. Activation of myosin phosphatase targeting subunit by mitosis-specific phosphorylation. J Cell Biol Feb. 22, 1999;144(4):735-44.

Panel 4D Summary: Ag3218/Ag3378—Two different experiments using probe/primer sets with the same sequence are in very good agreement. Highest expression is seen in the colon and a mucoepidermoid cell line (CTs=30-32). This gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AG. CG59241-01: Amiloride-Sensitive Sodium Channel

Expression of gene CG59241-01 was assessed using the primer-probe set Ag3407, described in Table AGA. Results of the RTQ-PCR runs are shown in Tables AGB, AGC and AGD.

Table AGA. Probe Name Ag3407

TABLE AGA
Probe Name Ag3407

| | | | Start | SEQ ID |
|---|---|---|---|---|
| Primers | Sequences | Length | Postion | NO: |
| Forward | 5′-gtcaccctctgcaacactaatg-3′ | 22 | 268 | 496 |
| Probe | TET-5′-ctgtcccagctcagctaccctgactt-3′-TAMRA | 26 | 298 | 497 |
| Reverse | 5′-tttcatccagtcccagcat-3′ | 19 | 340 | 498 |

TABLE AGB
CNS_neurodegeneration_v1.0

| | Rel. Exp. (%) Ag3407, | | Rel. Exp. (%) Ag3407, |
|---|---|---|---|
| Tissue Name | Run 210349883 | Tissue Name | Run 210349883 |
| AD 1 Hippo | 18.4 | Control (Path) 3 | 4.1 |
| | | Temporal Ctx | |
| AD 2 Hippo | 29.7 | Control (Path) 4 | 40.3 |
| | | Temporal Ctx | |
| AD 3 Hippo | 18.3 | AD 1 Occipital | 36.9 |
| | | Ctx | |
| AD 4 Hippo | 5.4 | AD 2 Occipital | 0.0 |
| | | Ctx (Missing) | |
| AD 5 hippo | 91.4 | AD 3 Occipital | 19.1 |
| | | Ctx | |
| AD 6 Hippo | 80.7 | AD 4 Occipital | 18.8 |
| | | Ctx | |
| Control 2 Hippo | 9.3 | AD 5 Occipital | 18.3 |
| | | Ctx | |
| Control 4 Hippo | 19.9 | AD 6 Occipital | 28.9 |
| | | Ctx | |
| Control (Path) 3 | 8.8 | Control 1 Occipital | 4.3 |
| Hippo | | Ctx | |
| AD 1 Temporal Ctx | 28.5 | Control 2 Occipital | 80.1 |
| | | Ctx | |
| AD 2 Temporal Ctx | 41.8 | Control 3 Occipital | 20.2 |
| | | Ctx | |
| AD 3 Temporal Ctx | 32.5 | Control 4 Occipital | 6.0 |
| | | Ctx | |
| AD 4 Temporal Ctx | 36.3 | Control (Path) 1 | 92.7 |
| | | Occipital Ctx | |
| AD 5 Inf Temporal | 100.0 | Control (Path) 2 | 25.3 |
| Ctx | | Occipital Ctx | |
| AD 5 SupTemporal | 56.6 | Control (Path) 3 | 3.0 |
| Ctx | | Occipital Ctx | |
| AD 6 Inf Temporal | 82.4 | Control (Path) 4 | 41.2 |
| Ctx | | Occipital Ctx | |
| AD 6 Sup Temporal | 44.1 | Control 1 Parietal | 21.9 |
| Ctx | | Ctx | |
| Control 1 Temporal | 15.3 | Control 2 Parietal | 79.0 |
| Ctx | | Ctx | |
| Control 2 Temporal | 24.1 | Control 3 Parietal | 22.2 |
| Ctx | | Ctx | |
| Control 3 Temporal | 34.6 | Control (Path) 1 | 77.9 |
| Ctx | | Parietal Ctx | |
| Control 4 Temporal | 12.0 | Control (Path) 2 | 47.6 |
| Ctx | | Parietal Ctx | |
| Control (Path) 1 | 53.6 | Control (Path) 3 | 6.2 |
| Temporal Ctx | | Parietal Ctx | |
| Control (Path) 2 | 56.6 | Control (Path) 4 | 67.4 |
| Temporal Ctx | | Parietal Ctx | |

TABLE AGC
General_screening_panel_v1.4

| | Rel. Exp. (%) | | Rel. Exp. (%) |
|---|---|---|---|
| | Ag3407, Run | | Ag3407, Run |
| Tissue Name | 216821458 | Tissue Name | 216821458 |
| Adipose | 0.2 | Renal ca. TK-10 | 16.6 |
| Melanoma* | 2.3 | Bladder | 0.3 |
| Hs688(A).T | | | |
| Melanoma* | 0.4 | Gastric ca. (liver met.) | 8.8 |
| Hs688(B).T | | NCI-N87 | |
| Melanoma* M14 | 2.0 | Gastric ca. KATO III | 0.7 |
| Melanoma* | 2.5 | Colon ca. SW-948 | 3.7 |
| LOXIMVI | | | |
| Melanoma* SK- | 8.7 | Colon ca. SW480 | 14.1 |
| MEL-5 | | | |
| Squamous cell | 1.2 | Colon ca.* (SW480 | 21.2 |
| carcinoma SCC-4 | | met) SW620 | |
| Testis Pool | 0.4 | Colon ca. HT29 | 10.7 |
| Prostate ca.* (bone | 4.4 | Colon ca. HCT-116 | 64.2 |
| met) PC-3 | | | |
| Prostate Pool | 2.3 | Colon ca. CaCo-2 | 32.3 |
| Placenta | 0.5 | Colon cancer tissue | 13.2 |
| Uterus Pool | 0.0 | Colon ca. SW1116 | 12.5 |
| Ovarian ca. | 8.4 | Colon ca. Colo-205 | 0.3 |
| OVCAR-3 | | | |
| Ovarian ca. SK- | 9.7 | Colon ca. SW-48 | 0.6 |
| OV-3 | | | |
| Ovarian ca. | 1.6 | Colon Pool | 2.8 |
| OVCAR-4 | | | |
| Ovarian ca. | 18.9 | Small Intestine Pool | 4.5 |
| OVCAR-5 | | | |
| Ovarian ca. | 4.9 | Stomach Pool | 1.4 |
| IGROV-1 | | | |
| Ovarian ca. | 5.9 | Bone Marrow Pool | 1.8 |
| OVCAR-8 | | | |
| Ovary | 2.0 | Fetal Heart | 2.4 |
| Breast ca. MCF-7 | 16.7 | Heart Pool | 0.3 |
| Breast ca. MDA- | 12.1 | Lymph Node Pool | 3.5 |
| MB-231 | | | |
| Breast ca. BT 549 | 22.7 | Fetal Skeletal Muscle | 1.9 |
| Breast ca. T47D | 27.4 | Skeletal Muscle Pool | 0.0 |
| Breast ca. MDA-N | 4.5 | Spleen Pool | 0.0 |
| Breast Pool | 2.9 | Thymus Pool | 2.1 |
| Trachea | 9.0 | CNS cancer | 0.9 |
| | | (glio/astro) U87-MG | |
| Lung | 0.0 | CNS cancer | 11.7 |
| | | (glio/astro) U-118-MG | |
| Fetal Lung | 10.8 | CNS cancer | 58.6 |
| | | (neuro; met) SK-N-AS | |
| Lung ca. NCI-N417 | 1.3 | CNS cancer (astro) SF- | 28.1 |
| | | 539 | |
| Lung ca. LX-1 | 21.8 | CNS cancer (astro) | 24.7 |
| | | SNB-75 | |
| Lung ca. NCI-H146 | 5.4 | CNS cancer (glio) | 7.3 |
| | | SNB-19 | |
| Lung ca. SHP-77 | 11.7 | CNS cancer (glio) SF- | 4.8 |
| | | 295 | |
| Lung ca. A549 | 8.0 | Brain (Amygdala) Pool | 3.9 |
| Lung ca. NCI-H526 | 0.0 | Brain (cerebellum) | 36.1 |
| Lung ca. NCI-H23 | 7.4 | Brain (fetal) | 100.0 |
| Lung ca. NCI-H460 | 5.4 | Brain (Hippocampus) | 5.6 |
| | | Pool | |
| Lung ca. HOP-62 | 2.9 | Cerebral Cortex Pool | 5.6 |
| Lung ca. NCI-H522 | 8.5 | Brain (Substantia | 7.1 |
| | | nigra) Pool | |
| Liver | 0.0 | Brain (Thalamus) Pool | 11.3 |
| Fetal Liver | 0.0 | Brain (whole) | 13.4 |
| Liver ca. HepG2 | 0.8 | Spinal Cord Pool | 12.7 |
| Kidney Pool | 2.1 | Adrenal Gland | 0.0 |
| Fetal Kidney | 3.7 | Pituitary gland Pool | 0.0 |
| Renal ca. 786-0 | 1.7 | Salivary Gland | 0.9 |
| Renal ca. A498 | 0.7 | Thyroid (female) | 0.0 |
| Renal ca. ACHN | 1.9 | Pancreatic ca. | 2.3 |
| | | CAPAN2 | |
| Renal ca. UO-31 | 0.2 | Pancreas Pool | 2.6 |

TABLE AGD
Panel 4D

| | Rel. Exp. (%) | | Rel. Exp. (%) |
|---|---|---|---|
| | Ag3407, Run | | Ag3407, Run |
| Tissue Name | 165296462 | Tissue Name | 165296462 |
| Secondary Th1 act | 7.9 | HUVEC IL-1beta | 0.0 |
| Secondary Th2 act | 17.1 | HUVEC IFN gamma | 0.0 |
| Secondary Tr1 act | 40.1 | HUVEC TNF alpha + | 0.0 |
| | | IFN gamma | |
| Secondary Th1 rest | 4.4 | HUVEC TNF alpha + | 0.0 |
| | | IL4 | |
| Secondary Th2 rest | 7.0 | HUVEC IL-11 | 0.0 |
| Secondary Tr1 rest | 11.7 | Lung Microvascular EC | 0.0 |
| | | none | |
| Primary Th1 act | 61.1 | Lung Microvascular EC | 0.0 |
| | | TNF alpha + IL-1beta | |
| Primary Th2 act | 69.3 | Microvascular Dermal | 0.0 |
| | | EC none | |
| Primary Tr1 act | 90.8 | Microsvasular Dermal | 0.0 |
| | | EC TNF alpha + IL-1beta | |
| Primary Th1 rest | 20.0 | Bronchial epithelium | 0.0 |
| | | TNF alpha + IL1beta | |
| Primary Th2 rest | 42.6 | Small airway epithelium | 3.0 |
| | | none | |
| Primary Tr1 rest | 52.5 | Small airway epithelium | 0.0 |
| | | TNF alpha + IL-1beta | |
| CD45RA CD4 | 2.8 | Coronery artery SMC rest | 3.6 |
| lymphocyte act | | | |
| CD45RO CD4 | 14.0 | Coronery artery SMC | 0.0 |
| lymphocyte act | | TNF alpha + IL-1beta | |
| CD8 lymphocyte act | 5.8 | Astrocytes rest | 11.6 |
| Secondary CD8 | 18.9 | Astrocytes TNF alpha + | 0.0 |
| lymphocyte rest | | IL-1beta | |
| Secondary CD8 | 22.2 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 4.5 | CCD1106 | 2.7 |
| CD95 CH11 | | (Keratinocytes) none | |
| LAK cells rest | 3.3 | CCD1106 | 0.0 |
| | | (Keratinocytes) | |
| | | TNF alpha + IL-1beta | |
| LAK cells IL-2 | 4.0 | Liver cirrhosis | 13.5 |
| LAK cells IL-2 + IL-12 | 5.7 | Lupus kidney | 4.1 |
| LAK cells IL-2 + IFN | 21.3 | NCI-H292 none | 9.0 |
| gamma | | | |
| LAK cells IL-2 + IL-18 | 6.7 | NCI-H292 IL-4 | 14.8 |
| LAK cells | 0.0 | NCI-H292 IL-9 | 3.5 |
| PMA/ionomycin | | | |
| NK Cells IL-2 rest | 0.0 | NCI-H292 IL-13 | 0.0 |
| Two Way MLR 3 day | 5.0 | NCI-H292 IFN gamma | 5.5 |
| Two Way MLR 5 day | 2.3 | HPAEC none | 0.0 |
| Two Way MLR 7 day | 8.2 | HPAEC TNF alpha + IL- | 0.0 |
| | | 1beta | |
| PBMC rest | 0.0 | Lung fibroblast none | 2.8 |
| PBMC PWM | 21.3 | Lung fibroblast TNF | 0.0 |
| | | alpha + IL-1beta | |
| PBMC PHA-L | 20.4 | Lung fibroblast IL-4 | 0.0 |
| Ramos (B cell) none | 0.0 | Lung fibroblast IL-9 | 0.0 |
| Ramos (B cell) | 2.8 | Lung fibroblast IL-13 | 0.0 |
| ionomycin | | | |
| B lymphocytes PWM | 100.0 | Lung fibroblast IFN | 1.4 |
| | | gamma | |
| B lymphocytes CD40L | 19.8 | Dermal fibroblast | 34.4 |
| and IL-4 | | CCD1070 rest | |
| EOL-1 dbcAMP | 2.6 | Dermal fibroblast | 68.8 |
| | | CCD1070 TNF alpha | |
| EOL-1 dbcAMP | 6.2 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 14.1 |
| Dendritic cells anti- | 6.0 | IBD Colitis 2 | 0.0 |
| CD40 | | | |
| Monocytes rest | 0.0 | IBD Crohn's | 0.0 |
| Monocytes LPS | 6.5 | Colon | 42.3 |
| Macrophages rest | 0.0 | Lung | 35.8 |
| Macrophages LPS | 0.0 | Thymus | 45.4 |
| HUVEC none | 0.0 | Kidney | 55.1 |
| HUVEC starved | 0.0 | | |

CNS_neurodegeneration_v1.0 Summary: Ag3407 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3407 Highest expression of the CG59241-01 gene is seen in fetal brain (CT=31.3). Furthermore, low to moderate levels of expression is also observed in CNS cancer cell lines (CTs=32-34). The CG59241-01 gene codes for a putative amiloride-sensitive sodium channel. A similar amiloride-sensitive sodium channel was shown to be highly expressed in malignant glioblastoma multiforme tumors and to be a charachteristic feature of malignant brain tumor cells (Ref. 1). Therefore, therapeutic modulation of the activity of the protein encoded by this gene may be beneficial in the treatment of CNS cancer. Significant expression is also seen in a cluster of cell lines derived from brain, colon, breast, and ovarian cancers. Therefore, therapeutic modulation of the activity of this gene or its protein product, through the use of small molecule drugs, protein therapeutics or antibodies, might be beneficial in the treatment of these cancers.

In addition, this gene is expressed at low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

### REFERENCES

1. Bubien J K, Keeton D A, Fuller C M, Gillespie G Y, Reddy A T, Mapstone T B, Benos D J. (1999) Malignant human gliomas express an amiloride-sensitive Na+ conductance. Am J Physiol 276(6 Pt 1):C1405-10

Panel 4D Summary: Ag3407 Highest expression Of the CG59241-01 gene is detected in PWM treated B lymphocytes (CT=32). Similar expression is also detected in primary activated Th1, Th2 and Tr1 cells, as well as TNF alpha treated dermal fibroblast CCD1070 cells (CTs=32). Therefore, expression of this gene can be used to distinguish these samples from other samples in the panel. Furthermore, this gene is expressed in activated lymphocytes. Likewise, no expression of this gene is seen in PBMC that contain normal B cells (CTr=40), but it is induced when PBMC are treated with the pokeweed mitogen or PHA-L (CTs=34). In addition, the transcript is not seen in the B cell lymphoma Ramos regardless of stimulation. Therefore, the gene product could potentially be used therapeutically in the treatment of Crohn's disease, ulcerative colitis, multiple sclerosis, chronic obstructive pulmonary disease, asthma, emphysema, rheumatoid arthritis, lupus erythematosus, psoriasis and in other diseases in which T cells and B cells are activated.

In addition, low expression of this gene is also observed in normal colon, lung, thymus and kidney tissues. The CG59241-01 gene encodes an amiloride-sensitive sodium channel, A similar channel, the amiloride-sensitive epithelial sodium channel (ENaC) constitutes the limiting step for sodium reabsorption in epithelial cells that line the distal nephron, distal colon, ducts of several exocrine glands and lung airways and plays an important role in pathophysiological and clinical conditions such as hypertension or lung edema. ENaC has been implicated in two genetic diseases, Liddle's syndrome and pseudoeiypoaldosteronism (PHA-1) (Ref. 1). Therefore, antibody or small molecule therapies designed with the protein encoded for by CG59241-01 gene could modulate kidney/colon/lung function and be important in the treatment of inflammatory or autoimmune diseases of these tissues in addition to hypertension, lung edema, Liddle's syndrom and PHA-1.

### REFERENCE

1. Hummler E. (11998) Reversal of convention: from man to experimental animal in elucidating the function of the renal amiloride-sensitive sodium channel. Exp Nephrol July-August 1998;6(4):265-71

AH. CG58602-01: FAD Binding Domain Containing Protein

Expression of gene CG58602-01 was assessed using the primer-probe set Ag3385, described in Table AHA. Results of the RTQ-PCR runs are shown in Tables AHB, AHC and AHD.

Table AHA. Probe Name Ag3385

TABLE AHA
Probe Name Ag3385

| | | | Start | SEQ ID |
|---|---|---|---|---|
| Primers | Sequences | Length | Position | NO: |
| Forward | 5′-tcatgaatccaggcaaagtg-3′ | 20 | 1427 | 499 |
| Probe | TET-5′-ttagcccacaagttccctgactacgg-3′-TAMRA | 26 | 1468 | 500 |
| Reverse | 5′-tggcatgaagaaaagttcca-3′ | 20 | 1503 | 501 |

TABLE AHB
CNS_neurodegeneration_v1.0

| | Rel. Exp. (%) Ag3385, | | Rel. Exp. (%) Ag3385, |
|---|---|---|---|
| Tissue Name | Run 210154892 | Tissue Name | Run 210154892 |
| AD 1 Hippo | 34.6 | Control (Path) 3 | 21.2 |
| | | Temporal Ctx | |
| AD 2 Hippo | 47.6 | Control (Path) 4 | 36.1 |
| | | Temporal Ctx | |
| AD 3 Hippo | 11.9 | AD 1 Occipital Ctx | 28.1 |
| AD 4 Hippo | 24.3 | AD 2 Occipital Ctx | 0.0 |
| | | (Missing) | |
| AD 5 Hippo | 56.3 | AD 3 Occipital Ctx | 15.0 |
| AD 6 Hippo | 63.3 | AD 4 Occipital Ctx | 34.9 |
| Control 2 Hippo | 42.6 | AD 5 Occipital Ctx | 52.1 |
| Control 4 Hippo | 24.7 | AD 6 Occipital Ctx | 25.3 |
| Control (Path) 3 | 23.3 | Control 1 Occipital | 14.3 |
| Hippo | | Ctx | |
| AD 1 Temporal | 23.8 | Control 2 Occipital | 69.3 |
| Ctx | | Ctx | |
| AD 2 Temporal | 73.7 | Control 3 Occipital | 29.5 |
| Ctx | | Ctx | |
| AD 3 Temporal | 7.3 | Control 4 Occipital | 14.9 |
| Ctx | | Ctx | |
| AD 4 Temporal | 39.0 | Control (Path) 1 | 68.3 |
| Ctx | | Occipital Ctx | |
| AD 5 Inf Temporal | 100.0 | Control (Path) 2 | 11.0 |
| Ctx | | Occipital Ctx | |
| AD 5 Sup | 55.5 | Control (Path) 3 | 8.9 |
| Temporal Ctx | | Occipital Ctx | |
| AD 6 Inf Temporal | 64.2 | Control (Path) 4 | 17.3 |
| Ctx | | Occipital Ctx | |
| AD 6 Sup | 54.0 | Control 1 Parietal | 32.8 |
| Temporal Ctx | | Ctx | |
| Control 1 | 23.8 | Control 2 Parietal | 62.0 |
| Temporal Ctx | | Ctx | |
| Control 2 | 50.3 | Control 3 Parietal | 33.4 |
| Temporal Ctx | | Ctx | |
| Control 3 | 38.4 | Control (Path) 1 | 70.7 |
| Temporal Ctx | | Parietal Ctx | |
| Control 3 | 19.2 | Control (Path) 2 | 31.4 |
| Temporal Ctx | | Parietal Ctx | |
| Control (Path) 1 | 56.6 | Control (Path) 3 | 20.9 |
| Temporal Ctx | | Parietal Ctx | |
| Control (Path) 2 | 47.6 | Control (Path) 4 | 43.2 |
| Temporal Ctx | | Parietal Ctx | |

TABLE AHC
General_screening_panel_v1.4

| | Rel. Exp. (%) | | Rel. Exp. (%) |
|---|---|---|---|
| | Ag3385, Run | | Ag3385, Run |
| Tissue Name | 217043538 | Tissue Name | 217043538 |
| Adipose | 2.4 | Renal ca. TK-10 | 3.5 |
| Melanoma* | 0.7 | Bladder | 6.6 |
| Hs688(A).T | | | |
| Melanoma* | 1.1 | Gastric ca. (liver met.) | 2.1 |
| Hs688(B).T | | NCI-N87 | |
| Melanoma* M14 | 0.9 | Gastric ca. KATO III | 0.9 |
| Melanoma* | 1.3 | Colon ca. SW-948 | 4.5 |
| LOXIMVI | | | |
| Melanoma* SK- | 2.2 | Colon ca. SW480 | 0.8 |
| MEL-5 | | | |
| Squamous cell | 0.1 | Colon ca.* (SW480 | 1.3 |
| carcinoma SCC-4 | | met) SW620 | |
| Testis Pool | 1.3 | Colon ca. HT29 | 0.6 |
| Prostate ca.* (bone | 5.8 | Colon ca. HCT-116 | 1.9 |
| met) PC-3 | | | |
| Prostate Pool | 4.0 | Colon ca. CaCo-2 | 28.5 |
| Placenta | 2.5 | Colon cancer tissue | 2.0 |
| Uterus Pool | 0.5 | Colon ca. SW1116 | 0.9 |
| Ovarian ca. | 1.1 | Colon ca. Colo-205 | 3.5 |
| OVCAR-3 | | | |
| Ovarian ca. SK- | 3.7 | Colon ca. SW-48 | 4.2 |
| OV-3 | | | |
| Ovarian ca. | 0.2 | Colon Pool | 3.0 |
| OVCAR-4 | | | |
| Ovarian ca. | 42.0 | Small Intestine Pool | 3.5 |
| OVCAR-5 | | | |
| Ovarian ca. | 8.0 | Stomach Pool | 1.8 |
| IGROV-1 | | | |
| Ovarian ca. | 2.7 | Bone Marrow Pool | 0.9 |
| OVCAR-8 | | | |
| Ovary | 3.3 | Fetal Heart | 12.9 |
| Breast ca. MCF-7 | 10.3 | Heart Pool | 8.3 |
| Breast ca. MDA- | 3.0 | Lymph Node Pool | 3.5 |
| MB-231 | | | |
| Breast ca. BT 549 | 1.3 | Fetal Skeletal Muscle | 2.6 |
| Breast ca. T47D | 100.0 | Skeletal Muscle Pool | 25.5 |
| Breast ca. MDA-N | 0.4 | Spleen Pool | 0.2 |
| Breast Pool | 3.1 | Thymus Pool | 2.7 |
| Trachea | 3.2 | CNS cancer | 4.0 |
| | | (glio/astro) U87-MG | |
| Lung | 2.9 | CNS cancer | 1.3 |
| | | (glio/astro) U-118-MG | |
| Fetal Lung | 3.0 | CNS cancer | 1.8 |
| | | (neuro; met) SK-N-AS | |
| Lung ca. NCI-N417 | 0.2 | CNS cancer (astro) SF- | 1.3 |
| | | 539 | |
| Lung ca. LX-1 | 1.1 | CNS cancer (astro) | 0.9 |
| | | SNB-75 | |
| Lung ca. NCI-H146 | 0.4 | CNS cancer (glio) | 5.0 |
| | | SNB-19 | |
| Lung ca. SHP-77 | 3.1 | CNS cancer (glio) SF- | 5.5 |
| | | 295 | |
| Lung ca. A549 | 4.3 | Brain (Amygdala) Pool | 5.5 |
| Lung ca. NCI-H526 | 0.4 | Brain (cerebellum) | 13.5 |
| Lung ca. NCI-H23 | 6.8 | Brain (fetal) | 5.6 |
| Lung ca. NCI-H460 | 1.5 | Brain Hippocampus) | 5.2 |
| | | Pool | |
| Lung ca. HOP-62 | 0.1 | Cerebral Cortex Pool | 7.1 |
| Lung ca. NCI-H522 | 3.6 | Brain (Substantia | 11.5 |
| | | nigra) Pool | |
| Liver | 13.6 | Brain (Thalamus) Pool | 7.2 |
| Fetal Liver | 12.0 | Brain (whole) | 7.2 |
| Liver ca. HepG2 | 2.7 | Spinal Cord Pool | 4.8 |
| Kidney Pool | 6.2 | Adrenal Gland | 6.0 |
| Fetal Kidney | 4.0 | Pituitary gland Pool | 1.7 |
| Renal ca. 786-0 | 0.2 | Salivary Gland | 6.6 |
| Renal ca. A498 | 1.4 | Thyroid (female) | 5.2 |
| Renal ca. ACHN | 0.8 | Pancreatic ca. | 3.5 |
| | | CAPAN2 | |
| Renal ca. UO-31 | 0.9 | Pancreas Pool | 4.4 |

TABLE AHD
Panel 4D

| | Rel. Exp. (%) | | Rel. Exp. (%) |
|---|---|---|---|
| | Ag3385, Run | | Ag3385, Run |
| Tissue Name | 165296471 | Tissue Name | 165296471 |
| Secondary Th1 act | 1.2 | HUVEC IL-1beta | 0.0 |
| Secondary Th2 act | 3.6 | HUVEC IFN gamma | 3.7 |
| Secondary Tr1 act | 2.6 | HUVEC TNF alpha + | 0.7 |
| | | IFN gamma | |
| Secondary Th1 rest | 0.4 | HUVEC TNF alpha + | 2.2 |
| | | IL4 | |
| Secondary Th2 rest | 0.9 | HUVEC IL-11 | 1.3 |
| Secondary Tr1 rest | 0.4 | Lung Microvascular EC | 3.2 |
| | | none | |
| Primary Th1 act | 1.1 | Lung Microvascular EC | 1.5 |
| | | TNF alpha + IL-1beta | |
| Primary Th2 act | 0.7 | Microvascular Dermal | 3.0 |
| | | EC none | |
| Primary Tr1 act | 0.0 | Microsvasular Dermal | 0.0 |
| | | EC TNF alpha + IL-1beta | |
| Primary Th1 rest | 1.1 | Bronchial epithelium | 0.6 |
| | | TNF alpha + IL-1beta | |
| Primary Th2 rest | 0.5 | Small airway epithelium | 0.7 |
| | | none | |
| Primary Tr1 rest | 0.6 | Small airway epithelium | 0.8 |
| | | TNF alpha + IL-1beta | |
| CD45RA CD4 | 2.0 | Coronery artery SMC rest | 0.5 |
| lymphocyte act | | | |
| CD45RO CD4 | 3.7 | Coronery artery SMC | 2.0 |
| lymphocyte act | | TNF alpha + IL-1beta | |
| CD8 lymphocyte act | 0.9 | Astrocytes rest | 1.5 |
| Secondary CD8 | 2.7 | Astrocytes TNF alpha + | 2.6 |
| lymphocyte rest | | IL-1beta | |
| Secondary CD8 | 0.0 | KU-812 (Basophil) rest | 3.0 |
| lymphocyte act | | | |
| CD4 lymphocyte none | 0.0 | KU-812 (Basophil) | 8.2 |
| | | PMA/ionomycin | |
| 2ry Th1/Th2/Tr1_anti- | 0.0 | CCD1106 | 3.3 |
| CD95 CH11 | | (Keratinocytes) none | |
| LAK cells rest | 9.4 | CCD1106 | 0.3 |
| | | (Keratinocytes) | |
| | | TNF alpha + IL-1beta | |
| LAK cells IL-2 | 0.8 | Liver cirrhosis | 7.9 |
| LAK cells IL-2 + IL-12 | 1.5 | Lupus kidney | 2.3 |
| LAK cells IL-2 + IFN | 3.7 | NCI-H292 none | 3.3 |
| gamma | | | |
| LAK cells IL-2 + IL-18 | 2.5 | NCI-H292 IL-4 | 8.4 |
| LAK cells | 2.0 | NCI-H292 IL-9 | 2.6 |
| PMA/ionomycin | | | |
| NK Cells IL-2 rest | 0.7 | NCI-H292 IL-13 | 2.9 |
| Two Way MLR 3 day | 4.6 | NCI-H292 IFN gamma | 1.8 |
| Two Way MLR 5 day | 2.8 | HPAEC none | 2.3 |
| Two Way MLR 7 day | 1.8 | HPAEC TNF alpha + IL- | 1.9 |
| | | 1beta | |
| PBMC rest | 0.6 | Lung fibroblast none | 1.5 |
| PBMC PWM | 11.0 | Lung fibroblast TNF | 0.7 |
| | | alpha + IL-1beta | |
| PBMC PHA-L | 2.3 | Lung fibroblast IL-4 | 1.6 |
| Ramos (B cell) none | 0.0 | Lung fibroblast IL-9 | 2.0 |
| Ramos (B cell) | 0.9 | Lung fibroblast IL-13 | 0.9 |
| ionomycin | | | |
| B lymphocytes PWM | 3.5 | Lung fibroblast IFN | 0.7 |
| | | gamma | |
| B lymphocytes CD40L | 5.4 | Dermal fibroblast | 1.6 |
| and IL-4 | | CCD1070 rest | |
| EOL-1 dbcAMP | 5.0 | Dermal fibroblast | 0.0 |
| | | CCD1070 TNF alpha | |
| EOL-1 dbcAMP | 1.2 | Dermal fibroblast | 2.3 |
| PMA/ionomycin | | CCD1070 IL-1beta | |
| Dendritic cells none | 15.5 | Dermal fibroblast IFN | 0.5 |
| | | gamma | |
| Dendritic cells LPS | 4.5 | Dermal fibroblast IL-4 | 0.4 |
| Dendritic cells anti- | 11.7 | IBD Colitis 2 | 0.3 |
| CD40 | | | |
| Monocytes rest | 8.7 | IBD Crohn's | 0.0 |
| Monocytes LPS | 0.6 | Colon | 5.1 |
| Macrophages rest | 13.5 | Lung | 6.7 |
| Macrophages LPS | 1.6 | Thymus | 100.0 |
| HUVEC none | 0.6 | Kidney | 11.3 |
| HUVEC starved | 1.7 | | |

CNS_neurodegeneration_v1.0 Summary: Ag3385 This panel confirms the expression of CG58602-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3385 Highest expression of the CG58602-01 gene is seen in a breast cancer cell line (CT=26.3). Significant expression is also seen in an ovarian cancer cell line. Thus, expression of this gene could be used to differentiate between these samples and other samples on this panel and as a marker to detect the presence of breast and ovarian cancers. Furthermore, therapeutic modulation of the expression or function of this gene may be effective in the treatment of breast and ovarian cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

Expression of this gene is higher in fetal skeletal muscle (CT=28.3) when compared to expression in adult skeletal muscle (CT=31.5). Thus, expression of this gene could be used to distinguish fetal from adult skeletal muscle.

In addition, this gene is expressed at high levels (CTs=29-30.4) in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3385 Highest expression of the CG58602-01 gene is seen in the thymus (CT=28). Thus, the putative protein encoded for by this gene could therefore play an important role in T cell development. Therefore, small molecule therapeutics designed against the proetin encoded by this gene could be utilized to modulate immune function (T cell development) and be important for organ transplant, AIDS treatment or post chemotherapy immune reconstitiution.

AI. CG58468-01: Serum Amyloid P Component

Expression of gene CG58468-01 was assessed using the primer-probe set Ag3356, described in Table AIA. Results of the RTQ-PCR runs are shown in Table AIB.

Table AIA. Probe Name Ag3356

TABLE AIA
Probe Name Ag3356

| | | | Start | SEQ ID |
|---|---|---|---|---|
| Primers | Sequences | Length | Position | NO: |
| Forward | 5′-aggcatttattttccctcaaga-3′ | 22 | 106 | 502 |
| Probe | TET-5′-agtctatgtgtccctgatccccaagg-3′-TAMRA | 26 | 137 | 503 |
| Reverse | 5′-gttttcaggcaaagcttgaagt-3′ | 22 | 181 | 504 |

TABLE AIB
General_screening_panel_v1.4

| | Rel. Exp. (%) | | Rel. Exp. (%) |
|---|---|---|---|
| | Ag3356, Run | | Ag3356, Run |
| Tissue Name | 216523476 | Tissue Name | 216523476 |
| Adipose | 2.2 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 1.7 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 100.0 |
| OVCAR-4 | | | |
| Ovarian ca. | 0.0 | Small Intestine Pool | 5.6 |
| OVCAR-5 | | | |
| Ovarian ca. | 0.0 | Stomach Pool | 0.0 |
| IGROV-1 | | | |
| Ovarian ca. | 0.0 | Bone Marrow Pool | 10.7 |
| OVCAR-8 | | | |
| Ovary | 0.0 | Fetal Heart | 0.0 |
| Breast ca. MCF-7 | 0.0 | Heart Pool | 2.6 |
| Breast ca. MDA- | 0.0 | Lymph Node Pool | 25.9 |
| MB-231 | | | |
| Breast ca. BT 549 | 0.0 | Fetal Skeletal Muscle | 2.1 |
| Breast ca. T47D | 0.0 | Skeletal Muscle Pool | 0.0 |
| Breast ca. MDA-N | 0.0 | Spleen Pool | 0.0 |
| Breast Pool | 19.6 | Thymus Pool | 0.0 |
| Trachea | 1.5 | CNS cancer | 0.0 |
| | | (glio/astro) U87-MG | |
| Lung | 0.0 | CNS cancer | 0.0 |
| | | (glio/astro) U-118-MG | |
| Fetal Lung | 5.0 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 38.7 | Brain (fetal) | 2.6 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 2.3 | Brain (Thalamus) Pool | 0.0 |
| Fetal Liver | 0.0 | Brain (whole) | 0.0 |
| Liver ca. HepG2 | 0.0 | Spinal Cord Pool | 2.1 |
| Kidney Pool | 19.1 | Adrenal Gland | 0.0 |
| Fetal Kidney | 0.0 | Pituitary gland Pool | 2.1 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 7.2 |

CNS_neurodegeneration_v1.0 Summary: Ag3356 Expression of the CG58468-01 gene is low/undetectable in all the samples on this panel. (CTs>35). (Data not shown.)

General_screening_panel_v1.4 Summary: Ag3356 Expression of the CG58468-01 gene is restricted to the colon (CT=34). Thus, expression of this gene could be used to differentiate between this sample and other samples on this panel.

Panel 4D Summary: Ag3356 Results from one experiment with the CG56003-01 gene are not included. The amp plot indicates that there were experimental difficulties with this run.

AJ. CG58183-01: N-Methyl-D-Aspartate Receptor

Expression of gene CG58183-01 was assessed using the primer-probe set Ag3355, described in Table AJA. Results of the RTQ-PCR runs are shown in Tables AJB, AJC and AJD.

Table AJA. Probe Name Ag3355

TABLE AJA
Probe Name Ag3355

| | | | Start | SEQ ID |
|---|---|---|---|---|
| Primers | Sequences | Length | Position | NO: |
| Forward | 5′-gctggccaactctgtctagac-3′ | 21 | 1617 | 505 |
| Probe | TET-5′-tgactcttccacattggacagccttt-3′-TAMRA | 26 | 1649 | 506 |
| Reverse | 5′-ttactgctatggaggctgctaa-3′ | 22 | 1675 | 507 |

TABLE AJB
CNS_neurodegeneration_v1.0

| | Rel. Exp. (%) Ag3355, | | Rel. Exp. (%) Ag3355, |
|---|---|---|---|
| Tissue Name | Run 210142850 | Tissue Name | Run 210142850 |
| AD 1 Hippo | 17.7 | Control (Path) 3 | 7.3 |
| | | Temporal Ctx | |
| AD 2 Hippo | 27.4 | Control (Path) 4 | 47.6 |
| | | Temporal Ctx | |
| AD 3 Hippo | 8.8 | AD 1 Occipital Ctx | 18.8 |
| AD 4 Hippo | 16.2 | AD 2 Occipital Ctx | 0.0 |
| | | (Missing) | |
| AD 5 Hippo | 53.6 | AD 3 Occipital Ctx | 3.0 |
| AD 6 Hippo | 51.4 | AD 4 Occipital Ctx | 27.2 |
| Control 2 Hippo | 41.8 | AD 5 Occipital Ctx | 55.1 |
| Control 4 Hippo | 10.4 | AD 6 Occipital Ctx | 6.3 |
| Control (Path) 3 | 4.5 | Control 1 Occipital | 2.8 |
| Hippo | | Ctx | |
| AD 1 Temporal | 18.3 | Control 2 Occipital | 39.0 |
| Ctx | | Ctx | |
| AD 2 Temporal | 48.0 | Control 3 Occipital | 18.2 |
| Ctx | | Ctx | |
| AD 3 Temporal | 5.7 | Control 4 Occipital | 3.4 |
| Ctx | | Ctx | |
| AD 4 Temporal | 15.2 | Control (Path) 1 | 81.8 |
| Ctx | | Occipital Ctx | |
| AD 5 Inf | 61.6 | Control (Path) 2 | 9.0 |
| Temporal Ctx | | Occipital Ctx | |
| AD 5 Sup | 69.3 | Control (Path) 3 | 0.0 |
| Temporal Ctx | | Occipital Ctx | |
| AD 6 Inf | 66.9 | Control (Path) 4 | 13.3 |
| Temporal Ctx | | Occipital Ctx | |
| AD 6 Sup | 62.9 | Control 1 Parietal | 6.6 |
| Temporal Ctx | | Ctx | |
| Control 1 | 8.5 | Control 2 Parietal | 74.7 |
| Temporal Ctx | | Ctx | |
| Control 2 | 66.9 | Control 3 Parietal | 21.0 |
| Temporal Ctx | | Ctx | |
| Control 3 | 34.9 | Control (Path) 1 | 100.0 |
| Temporal Ctx | | Parietal Ctx | |
| Control 3 | 7.0 | Control (Path) 2 | 21.9 |
| Temporal Ctx | | Parietal Ctx | |
| Control (Path) 1 | 90.1 | Control (Path) 3 | 6.0 |
| Temporal Ctx | | Parietal Ctx | |
| Control (Path) 2 | 74.7 | Control (Path) 4 | 50.7 |
| Temporal Ctx | | Parietal Ctx | |

TABLE AJC
General_screening_panel_v1.4

| | Rel. Exp. (%) | | Rel. Exp. (%) |
|---|---|---|---|
| | Ag3355, Run | | Ag3355, Run |
| Tissue Name | 216523475 | Tissue Name | 216523475 |
| Adipose | 0.0 | Renal ca. TK-10 | 0.0 |
| Melanoma* | 0.9 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.2 |
| MEL-5 | | | |
| Squamous cell | 0.0 | Colon ca.* (SW480 | 0.0 |
| carcinoma SCC-4 | | met) SW620 | |
| Testis Pool | 2.4 | Colon ca. HT29 | 0.0 |
| Prostate ca.* (bone | 0.0 | Colon ca. HCT-116 | 0.0 |
| met) PC-3 | | | |
| Prostate Pool | 2.1 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.4 | Colon ca. Colo-205 | 0.0 |
| OVCAR-3 | | | |
| Ovarian ca. SK- | 0.3 | Colon ca. SW-48 | 0.0 |
| OV-3 | | | |
| Ovarian ca. | 0.0 | Colon Pool | 2.2 |
| OVCAR-4 | | | |
| Ovarian ca. | 0.0 | Small Intestine Pool | 3.4 |
| OVCAR-5 | | | |
| Ovarian ca. | 0.0 | Stomach Pool | 2.4 |
| IGROV-1 | | | |
| Ovarian ca. | 0.0 | Bone Marrow Pool | 1.5 |
| OVCAR-8 | | | |
| Ovary | 4.4 | Fetal Heart | 1.9 |
| Breast ca. MCF-7 | 0.0 | Heart Pool | 3.6 |
| Breast ca. MDA- | 0.0 | Lymph Node Pool | 2.9 |
| MB-231 | | | |
| Breast ca. BT 549 | 0.0 | Fetal Skeletal Muscle | 1.8 |
| Breast ca. T47D | 0.0 | Skeletal Muscle Pool | 0.0 |
| Breast ca. MDA-N | 0.0 | Spleen Pool | 4.3 |
| Breast Pool | 5.7 | Thymus Pool | 4.7 |
| Trachea | 1.7 | CNS cancer | 0.0 |
| | | (glio/astro) U87-MG | |
| Lung | 1.6 | CNS cancer | 0.1 |
| | | (glio/astro) U-118-MG | |
| Fetal Lung | 0.0 | CNS cancer | 3.2 |
| | | (neuro; met) SK-N-AS | |
| Lung ca. NCI-N417 | 17.8 | CNS cancer (astro) SF- | 14.8 |
| | | 539 | |
| Lung ca. LX-1 | 0.0 | CNS cancer (astro) | 17.6 |
| | | SNB-75 | |
| Lung ca. NCI-H146 | 4.0 | CNS cancer (glio) | 0.0 |
| | | SNB-19 | |
| Lung ca. SHP-77 | 11.7 | CNS cancer (glio) SF- | 0.0 |
| | | 295 | |
| Lung ca. A549 | 0.0 | Brain (Amygdala) Pool | 27.7 |
| Lung ca. NCI-H526 | 0.3 | Brain (cerebellum) | 0.7 |
| Lung ca. NCI-H23 | 1.9 | Brain (fetal) | 100.0 |
| Lung ca. NCI-H460 | 0.3 | Brain (Hippocampus) | 33.0 |
| | | Pool | |
| Lung ca. HOP-62 | 0.3 | Cerebral Cortex Pool | 42.3 |
| Lung ca. NCI-H522 | 0.0 | Brain (Substantia | 43.8 |
| | | nigra) Pool | |
| Liver | 0.2 | Brain (Thalamus) Pool | 50.7 |
| Fetal Liver | 0.4 | Brain (whole) | 71.2 |
| Liver ca. HepG2 | 0.0 | Spinal Cord Pool | 15.0 |
| Kidney Pool | 1.4 | Adrenal Gland | 0.0 |
| Fetal Kidney | 7.2 | Pituitary gland Pool | 0.0 |
| Renal ca. 786-0 | 0.0 | Salivary Gland | 0.1 |
| Renal ca. A498 | 0.0 | Thyroid (female) | 0.1 |
| Renal ca. ACHN | 0.0 | Pancreatic ca. | 0.0 |
| | | CAPAN2 | |
| Renal ca. UO-31 | 0.0 | Pancreas Pool | 2.7 |

TABLE AJD
Panel 4D

| | Rel. Exp. (%) | | Rel. Exp. (%) |
|---|---|---|---|
| | Ag3355, Run | | Ag3355, Run |
| Tissue Name | 165241988 | Tissue Name | 165241988 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 11.8 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 57.8 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 67.4 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 39.8 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 12.7 |
| Macrophages rest | 0.0 | Lung | 15.2 |
| Macrophages LPS | 0.0 | Thymus | 100.0 |
| HUVEC none | 0.0 | Kidney | 73.2 |
| HUVEC starved | 0.0 | | |

CNS_neurodegeneration_v1.0 Summary: Ag3355 This panel confirms the expression of CG58183-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3355 Highest expression of CG58183-01 gene is detected in fetal brain (Ct=29.2). In addition, this gene is expressed at high levels in all regions of the central nervous system examined, including amygdala, hippocampus, substantia nigra, thalamus, cerebellum, cerebral cortex, and spinal cord (CTs=29-32). Therefore, this gene may play a role in central nervous system disorders such as Alzheimer's disease, Parkinson's disease, epilepsy, multiple sclerosis, schizophrenia and depression.

This gene codes for N-methyl-D-aspartate (NMDA) receptor 3A protein. In cats and rhodent models competitive NMDA receptor antagonists, such as D-(E)-4-(3-phosphonoprop-2-enyl)piperazine-2-carboxylic acid, which act at the neurotransmitter recognition site were shown to be effective in reducing ischaemic brain damage when administered prior to the onset of an ischaemic episode (Ref. 1). Therefore, therapeutic modulation of the activity of the protein encoded by this gene may be beneficial in the treatment of ischaemic brain.

Among tissues with metabolic or endocrine function, this gene is expressed at low levels in pancreas, heart, and the gastrointestinal tract. Therefore, therapeutic modulation of the activity of this gene may prove useful in the treatment of endocrine/metabolically related diseases, such as obesity and diabetes.

Furthermore, low to moderate expression of this gene is detected in lung cancer, and CNS3 cancer cell lines. Therefore, therapeutic modulation of the activity of this gene or its protein product, through the use of small molecule drugs, protein therapeutics or antibodies, might be beneficial in the treatment of lung cancer or CNS cancer.

### REFERENCES

1. McCulloch J. (1991) Ischaemic brain damage—prevention with competitive and non-comnpetitive antagonists of N-methyl-D-aspartate receptors. Arzneimittelforschung 41(3A):319-24.

Panel 4D Summary: Ag3355 Expression of the CG58183-01 gene is limited to a few samples, with highest expression in the thymus (CT=33.5). Thus, expression of this gene may be useful as a marker of thymic tissue. Low, but significant levels of expression are also seen in the kidney, in TNF-alpha and IL-1 beta treated astrocytes and in the PMA/ionomycin treated basophil cell line KU-812. Thus, this gene product may be involved in the normal homeostasis of this tissue. Therefore, agonistic antibodies or protein therapeutics may be important in the treatment of inflammatory or autoimmune diseases that affect the kidney, including lupus and glomerulonephritis. In addition, the expression of this transcript in astrocytes treated with TNF-a and IL-1 indicates that therapeutics designed against the protein encoded by this gene may be useful for the treatment of inflammatory CNS diseases such as multiple sclerosis.

AK. CG59315-01: connexin

Expression of gene CG59315-01 was assessed using the primer-probe set Ag3542, described in Table AKA. Results of the RTQ-PCR runs are shown in Tables AKB and AKC.

Table AKA. Probe Name Ag3542

TABLE AKA
Probe Name Ag3542

| | | | Start | SEQ ID |
|---|---|---|---|---|
| Primers | Sequences | Length | Position | NO: |
| Forward | 5′-ggacacctcccaacctagatc-3′ | 21 | 1024 | 508 |
| Probe | TET-5′-tacctgtcttccttccttgaggctgg-3′-TAMRA | 26 | 1046 | 509 |
| Reverse | 5′-ttgcattcttgtgtccatgag-3′ | 21 | 1081 | 510 |

TABLE AKB
General_screening_panel_v1.4

| | Rel. Exp. (%) | | Rel. Exp. (%) |
|---|---|---|---|
| | Ag3542, Run | | Ag3542, Run |
| Tissue Name | 217049297 | Tissue Name | 217049297 |
| Adipose | 17.3 | Renal ca. TK-10 | 6.8 |
| Melanoma* | 0.4 | Bladder | 2.5 |
| Hs688(A).T | | | |
| Melanoma* | 1.0 | Gastric ca. (liver met.) | 13.1 |
| Hs688(B).T | | NCI-N87 | |
| Melanoma* M14 | 12.2 | Gastric ca. KATO III | 12.2 |
| Melanoma* | 0.0 | Colon ca. SW-948 | 3.8 |
| LOXIMVI | | | |
| Melanoma* SK- | 0.7 | Colon ca. SW480 | 39.0 |
| MEL-5 | | | |
| Squamous cell | 2.0 | Colon ca.* (SW480 | 6.7 |
| carcinoma SCC-4 | | met) SW620 | |
| Testis Pool | 0.3 | Colon ca. HT29 | 2.3 |
| Prostate ca.* (bone | 0.0 | Colon ca. HCT-116 | 17.7 |
| met) PC-3 | | | |
| Prostate Pool | 0.0 | Colon ca. CaCo-2 | 2.8 |
| Placenta | 1.2 | Colon cancer tissue | 1.6 |
| Uterus Pool | 0.0 | Colon ca. SW1116 | 0.3 |
| Ovarian ca. | 6.3 | Colon ca. Colo-205 | 0.3 |
| OVCAR-3 | | | |
| Ovarian ca. SK- | 2.5 | Colon ca. SW-48 | 0.0 |
| OV-3 | | | |
| Ovarian ca. | 0.0 | Colon Pool | 1.7 |
| OVCAR-4 | | | |
| Ovarian ca. | 25.0 | Small Intestine Pool | 6.3 |
| OVCAR-5 | | | |
| Ovarian ca. | 6.4 | Stomach Pool | 5.4 |
| IGROV-1 | | | |
| Ovarian ca. | 0.0 | Bone Marrow Pool | 3.1 |
| OVCAR-8 | | | |
| Ovary | 0.6 | Fetal Heart | 1.7 |
| Breast ca. MCF-7 | 12.9 | Heart Pool | 1.5 |
| Breast ca. MDA- | 5.0 | Lymph Node Pool | 3.6 |
| MB-231 | | | |
| Breast ca. BT 549 | 8.7 | Fetal Skeletal Muscle | 0.0 |
| Breast ca. T47D | 100.0 | Skeletal Muscle Pool | 6.1 |
| Breast ca. MDA-N | 2.7 | Spleen Pool | 5.8 |
| Breast Pool | 4.9 | Thymus Pool | 3.0 |
| Trachea | 9.3 | CNS cancer | 1.0 |
| | | (glio/astro) U87-MG | |
| Lung | 0.0 | CNS cancer | 13.7 |
| | | (glio/astro) U-118-MG | |
| Fetal Lung | 3.2 | CNS cancer | 35.4 |
| | | (neuro; met) SK-N-AS | |
| Lung ca. NCI-N417 | 1.2 | CNS cancer (astro) SF- | 4.9 |
| | | 539 | |
| Lung ca. LX-1 | 11.7 | CNS cancer (astro) | 2.7 |
| | | SNB-75 | |
| Lung ca. NCI-H146 | 2.9 | CNS cancer (glio) | 1.4 |
| | | SNB-19 | |
| Lung ca. SHP-77 | 8.1 | CNS cancer (glio) SF- | 12.5 |
| | | 295 | |
| Lung ca. A549 | 10.8 | Brain (Amygdala) Pool | 0.4 |
| Lung ca. NCI-H526 | 2.1 | Brain (cerebellum) | 13.6 |
| Lung ca. NCI-H23 | 8.1 | Brain (fetal) | 6.9 |
| Lung ca. NCI-H460 | 0.8 | Brain (Hippocampus) | 1.5 |
| | | Pool | |
| Lung ca. HOP-62 | 10.2 | Cerebral Cortex Pool | 0.0 |
| Lung ca. NCI-H522 | 4.9 | Brain (Substantia | 0.2 |
| | | nigra) Pool | |
| Liver | 0.0 | Brain (Thalamus) Pool | 1.2 |
| Fetal Liver | 1.2 | Brain (whole) | 0.0 |
| Liver ca. HepG2 | 0.0 | Spinal Cord Pool | 0.4 |
| Kidney Pool | 3.1 | Adrenal Gland | 2.0 |
| Fetal Kidney | 0.0 | Pituitary gland Pool | 1.8 |
| Renal ca. 786-0 | 6.3 | Salivary Gland | 3.2 |
| Renal ca. A498 | 0.0 | Thyroid (female) | 3.8 |
| Renal ca. ACHN | 12.1 | Pancreatic ca. | 0.0 |
| | | CAPAN2 | |
| Renal ca. UO-31 | 3.2 | Pancreas Pool | 4.2 |

TABLE AKC
Panel 4D

| | Rel. Exp. (%) | | Rel. Exp. (%) |
|---|---|---|---|
| | Ag3542, Run | | Ag3542, Run |
| Tissue Name | 166453844 | Tissue Name | 166453844 |
| Secondary Th1 act | 3.9 | HUVEC IL-1beta | 0.0 |
| Secondary Th2 act | 5.4 | HUVEC IFN gamma | 2.4 |
| Secondary Tr1 act | 3.8 | HUVEC TNF alpha + | 0.4 |
| | | IFN gamma | |
| Secondary Th1 rest | 33.0 | HUVEC TNF alpha + | 0.0 |
| | | IL4 | |
| Secondary Th2 rest | 5.3 | HUVEC IL-11 | 1.4 |
| Secondary Tr1 rest | 14.8 | Lung Microvascular EC | 3.1 |
| | | none | |
| Primary Th1 act | 6.1 | Lung Microvascular EC | 1.9 |
| | | TNF alpha + IL-1beta | |
| Primary Th2 act | 0.6 | Microvascular Dermal | 1.9 |
| | | EC none | |
| Primary Tr1 act | 5.5 | Microsvasular Dermal | 0.0 |
| | | EC TNF alpha + IL-1beta | |
| Primary Th1 rest | 84.7 | Bronchial epithelium | 2.7 |
| | | CCD1070 TNF alpha | |
| EOL-1 dbcAMP | 11.7 | Dermal fibroblast | 0.0 |
| PMA/ionomycin | | CCD1070 IL-1beta | |
| Dendritic cells none | 6.3 | Dermal fibroblast IFN | 0.3 |
| | | gamma | |
| Dendritic cells LPS | 1.4 | Dermal fibroblast IL-4 | 1.4 |
| Dendritic cells anti- | 2.3 | IBD Colitis 2 | 4.0 |
| CD40 | | | |
| Monocytes rest | 53.2 | IBD Crohn's | 3.2 |
| Monocytes LPS | 19.2 | Colon | 100.0 |
| Macrophages rest | 0.6 | Lung | 11.1 |
| Macrophages LPS | 0.0 | Thymus | 2.7 |
| HUVEC none | 5.4 | Kidney | 7.7 |
| HUVEC starved | 4.3 | | |

CNS_neurodegeneration_v1.0 Summary: Ag3542 Expression of the CG59315-01 gene is low/undetectable in all the samples on this panel. (CTs>35). (Data not shown.)

General_screening_panel_v1.4 Summary: Ag3542 Expression of the CG59315-01 gene is highest in a breast cancer cell line (CT=31.3). Furthermore, there is significant expression in a cluster of cell lines derived from brain cancer, colon cancer and ovarian cancer. Therefore, expression of this gene could be used to differentiate between these samples and other samples on this panel and as a marker to detect the presence of colon, brain, ovarian, and breast cancer. Furthermore, therapeutic modulation of the expression or function of this gene may be effective in the treatment of colon, brain, ovarian, and breast cancers.

Low but significant levels of expression are also seen in the cerebellum. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among metabolic tissues, this gene is expressed at low levels in adipose. Therefore, this gene product may be useful in the treatment of obesity.

Panel 4D Summary: Ag3542 Expression of the CG59315-01 gene is highest in the normal colon (CT=30). Furthermore, expression is undetectable in colon samples from Crohn's and colitis patients. Thus, expression of this gene could be used to differentiate between normal and inflammed colon. This gene encodes a connexin homolog, a gap junction protein involved in intercellular communication.

The expression of this connexin-like protein in several of the resting and activated T lymphocyte preparations and in resting monocytes suggests that small molecule antagonists or therapeutic antibodies that block its function may also be useful in the treatment of a number of inflammatory and autoimmune diseases in which T cells and monocytes play a pivotal role. These include, but are not limited to, Crohn's disease, ulcerative colitis, multiple sclerosis, chronic obstructive pulmonary disease, asthma, emphysema, rheumatoid arthritis, lupus erythematosus, or psoriasis.

### REFERENCES

1. Kwak B R, Mulhaupt F, Veillard N, Gros D B, Mach F. Altered pattern of vascular connexin expression in atherosclerotic plaques. Arterioscler Thromb Vasc Biol Feb. 1, 2002;22(2):225-30

AL. CG59203-01: Lysozyme C-Like Protein

Expression of gene CG59203-01 was assessed using the primer-probe set Ag3392, described in Table ALA. Results of the RTQ-PCR runs are shown in Tables ALB and ALC.

Table ALA. Probe Name Ag3392

TABLE ALA
Probe Name Ag3392

| | | | Start | SEQ ID |
|---|---|---|---|---|
| Primers | Sequences | Length | Position | NO: |
| Forward | 5′-tgtgaggtttcctaaactggaa-3′ | 22 | 540 | 511 |
| Probe | TET-5′-ctttgcagcaacgccctagggttt-3′-TAMRA | 24 | 576 | 512 |
| Reverse | 5′-tgacacaggcatttggacat-3′ | 20 | 607 | 513 |

TABLE ALB
General_screening_panel_v1.4

| | Rel. Exp. (%) | | Rel. Exp. (%) |
|---|---|---|---|
| | Ag3392, Run | | Ag3392, Run |
| Tissue Name | 216821373 | Tissue Name | 216821373 |
| Adipose | 0.0 | Renal ca. TK-10 | 2.7 |
| Melanoma* | 0.0 | Bladder | 0.0 |
| Hs688(A).T | | | |
| Melanoma* | 0.0 | Gastric ca. (liver met.) | 0.7 |
| Hs688(B).T | | NCI-N87 | |
| Melanoma* M14 | 1.2 | Gastric ca. KATO III | 0.4 |
| Melanoma* | 0.0 | Colon ca. SW-948 | 0.6 |
| LOXIMVI | | | |
| Melanoma* SK- | 0.0 | Colon ca. SW480 | 2.0 |
| MEL-5 | | | |
| Squamous cell | 0.0 | Colon ca.* (SW480 | 4.7 |
| carcinoma SCC-4 | | met) SW620 | |
| Testis Pool | 100.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 1.2 |
| Placenta | 0.7 | Colon cancer tissue | 0.0 |
| Uterus Pool | 0.0 | Colon ca. SW1116 | 0.0 |
| Ovarian ca. | 0.4 | Colon ca. Colo-205 | 11.6 |
| OVCAR-3 | | | |
| Ovarian ca. SK- | 1.0 | Colon ca. SW-48 | 2.2 |
| OV-3 | | | |
| Ovarian ca. | 0.0 | Colon Pool | 1.1 |
| OVCAR-4 | | | |
| Ovarian ca. | 0.0 | Small Intestine Pool | 0.4 |
| OVCAR-5 | | | |
| Ovarian ca. | 0.0 | Stomach Pool | 0.0 |
| IGROV-1 | | | |
| Ovarian ca. | 0.0 | Bone Marrow Pool | 1.3 |
| OVCAR-8 | | | |
| Ovary | 0.0 | Fetal Heart | 0.0 |
| Breast ca. MCF-7 | 15.2 | Heart Pool | 1.1 |
| Breast ca. MDA- | 0.0 | Lymph Node Pool | 0.0 |
| MB-231 | | | |
| Breast ca. BT 549 | 1.7 | Fetal Skeletal Muscle | 0.0 |
| Breast ca. T47D | 0.0 | Skeletal Muscle Pool | 0.5 |
| Breast ca. MDA-N | 0.0 | Spleen Pool | 0.0 |
| Breast Pool | 0.0 | Thymus Pool | 0.9 |
| Trachea | 1.1 | CNS cancer | 1.4 |
| | | (glio/astro) U87-MG | |
| Lung | 0.0 | CNS cancer | 0.5 |
| | | (glio/astro) U-118-MG | |
| Fetal Lung | 0.8 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 5.4 | CNS cancer (astro) | 6.1 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 3.1 | Brain (Amygdala) Pool | 0.0 |
| Lung ca. NCI-H526 | 0.0 | Brain (cerebellum) | 0.0 |
| Lung ca. NCI-H23 | 0.9 | Brain (fetal) | 0.0 |
| Lung ca. NCI-H460 | 1.1 | Brain (Hippocampus) | 0.0 |
| | | Pool | |
| Lung ca. HOP-62 | 0.0 | Cerebral Cortex Pool | 0.5 |
| Lung ca. NCI-H522 | 0.0 | Brain (Substantia | 0.0 |
| | | nigra) Pool | |
| Liver | 1.5 | Brain (Thalamus) Pool | 0.9 |
| Fetal Liver | 1.2 | Brain (whole) | 0.0 |
| Liver ca. HepG2 | 18.3 | Spinal Cord Pool | 0.0 |
| Kidney Pool | 1.6 | Adrenal Gland | 0.0 |
| Fetal Kidney | 0.0 | Pituitary gland Pool | 0.0 |
| Renal ca. 786-0 | 0.3 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

TABLE ALC
Panel 4D

| | Rel. Exp. (%) | | Rel. Exp. (%) |
|---|---|---|---|
| | Ag3392, Run | | Ag3392, Run |
| Tissue Name | 165296470 | Tissue Name | 165296470 |
| Secondary Th1 act | 0.0 | HUVEC IL-1beta | 0.0 |
| Secondary Th2 act | 36.9 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 16.8 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 11.1 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 26.8 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 0.0 |
| Macropages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag3392 Expression of the CG59203-01 gene is low/undetectable in all samples on this panel (CTs>35). (Data not shown.)

General_screening_panel_v1.4 Summary: Ag3392 Highest expression of the CG59203-01 gene is seen in the testis. Thus, expression of this gene could be used as a marker of testicular tissue. Furthermore, therapeutic modulation of the expression or function of this gene may be effective in treating infertility or hypogonadism.

Panel 4D Summary: Ag3392 Significant expression of this gene is detected in a liver cirrhosis sample (CT=33.8). Furthermore, expression of this gene is not detected in normal liver in Panel 1.3D, suggesting that its expression is unique to liver cirrhosis. Therefore, therapeutic modulation of the expression or function of this gene may reduce or inhibit fibrosis that occurs in liver cirrhosis. In addition, expression of this gene could also be used for the diagnosis of liver cirrhosis.

AM. CG58662-01: Cytoplasmic Protein

Expression of gene CG58662-01 was assessed using the primer-probe set Ag3387, described in Table AMA. Results of the RTQ-PCR runs are shown in Tables AMB, AMC and AMD.

Table AMA. Probe Name Ag3387

TABLE AMA
Probe Name Ag3387

| | | | Start | SEQ ID |
|---|---|---|---|---|
| Primers | Sequences | Length | Position | NO: |
| Forward | 5′-aacctgcactcctccatga-3′ | 19 | 504 | 514 |
| Probe | TET-5′-agaccccagcagggtatcctctgag-3′-TAMRA | 25 | 532 | 515 |
| Reverse | 5′-ctctgtcagtgcccacatct-3′ | 20 | 564 | 516 |

TABLE AMB
CNS_neurodegeneration_v1.0

| | | | Rel. Exp. |
|---|---|---|---|
| | | | (%) |
| | Rel. Exp. (%) | | Ag3387, |
| | Ag3387, Run | | Run |
| Tissue Name | 210155038 | Tissue Name | 210155038 |
| AD 1 Hippo | 15.7 | Control (Path) 3 | 7.3 |
| | | Temporal Ctx | |
| AD 2 Hippo | 34.6 | Control (Path) 4 | 42.0 |
| | | Temporal Ctx | |
| AD 3 Hippo | 5.5 | AD 1 Occipital Ctx | 17.9 |
| AD 4 Hippo | 9.7 | AD 2 Occipital Ctx | 0.0 |
| | | (Missing) | |
| AD 5 Hippo | 95.3 | AD 3 Occipital Ctx | 5.4 |
| AD 6 Hippo | 33.9 | AD 4 Occipital Ctx | 19.6 |
| Control 2 Hippo | 41.5 | AD 5 Occipital Ctx | 61.1 |
| Control 4 Hippo | 9.0 | AD 6 Occipital Ctx | 19.5 |
| Control (Path) 3 | 6.7 | Control 1 Occipital | 5.8 |
| Hippo | | Ctx | |
| AD 1 Temporal | 11.2 | Control 2 Occipital | 83.5 |
| Ctx | | Ctx | |
| AD 2 Temporal | 37.6 | Control 3 Occipital | 19.3 |
| Ctx | | Ctx | |
| AD 3 Temporal | 4.0 | Control 4 Occipital | 5.0 |
| Ctx | | Ctx | |
| AD 4 Temporal | 21.5 | Control (Path) 1 | 88.9 |
| Ctx | | Occipital Ctx | |
| AD 5 Inf Temporal | 100.0 | Control (Path) 2 | 13.4 |
| Ctx | | Occipital Ctx | |
| AD 5 Sup | 37.9 | Control (Path) 3 | 5.8 |
| Temporal Ctx | | Occipital Ctx | |
| AD 6 Inf Temporal | 35.6 | Control (Path) 4 | 24.3 |
| Ctx | | Occipital Ctx | |
| AD 6 Sup | 39.2 | Control 1 Parietal | 9.4 |
| Temporal Ctx | | Ctx | |
| Control 1 | 6.7 | Control 2 Parietal | 44.4 |
| Temporal Ctx | | Ctx | |
| Control 2 | 65.5 | Control 3 Parietal | 28.5 |
| Temporal Ctx | | Ctx | |
| Control 3 | 19.3 | Control (Path) 1 | 90.8 |
| Temporal Ctx | | Parietal Ctx | |
| Control 3 | 11.4 | Control (Path) 2 | 25.7 |
| Temporal Ctx | | Parietal Ctx | |
| Control (Path) 1 | 83.5 | Control (Path) 3 | 5.6 |
| Temporal Ctx | | Parietal Ctx | |
| Control (Path) 2 | 56.6 | Control (Path) 4 | 56.6 |
| Temporal Ctx | | Parietal Ctx | |

TABLE AMC
General_screening_panel_v1.4

| | Rel. Exp. (%) | | Rel. Exp. (%) |
|---|---|---|---|
| | Ag3387, | | Ag3387, |
| Tissue Name | Run 217043912 | Tissue Name | Run 217043912 |
| Adipose | 8.2 | Renal ca. TK-10 | 66.4 |
| Melanoma* Hs688(A).T | 30.6 | Bladder | 11.2 |
| Melanoma* | 34.6 | Gastric ca. (liver met.) | 15.3 |
| Hs688(B).T | | NCI-N87 | |
| Melanoma* M14 | 27.0 | Gastric ca. KATO III | 20.6 |
| Melanoma* LOXIMVI | 17.6 | Colon ca. SW-948 | 1.1 |
| Melanoma* SK- | 25.3 | Colon ca. SW480 | 33.0 |
| MEL-5 | | | |
| Squamous cell | 5.7 | Colon ca.* (SW480 | 29.9 |
| carcinoma SCC-4 | | met) SW620 | |
| Testis Pool | 17.8 | Colon ca. HT29 | 8.8 |
| Prostate ca.* (bone | 27.7 | Colon ca. HCT-116 | 15.4 |
| met) PC-3 | | | |
| Prostate Pool | 16.0 | Colon ca. CaCo-2 | 13.1 |
| Placenta | 11.0 | Colon cancer tissue | 13.8 |
| Uterus Pool | 1.7 | Colon ca. SW1116 | 7.9 |
| Ovarian ca. | 17.0 | Colon ca. Colo-205 | 4.4 |
| OVCAR-3 | | | |
| Ovarian ca. SK- | 11.3 | Colon ca. SW-48 | 9.3 |
| OV-3 | | | |
| Ovarian ca. | 7.1 | Colon Pool | 20.4 |
| OVCAR-4 | | | |
| Ovarian ca. | 37.4 | Small Intestine Pool | 12.5 |
| OVCAR-5 | | | |
| Ovarian ca. | 23.7 | Stomach Pool | 12.1 |
| IGROV-1 | | | |
| Ovarian ca. | 16.8 | Bone Marrow Pool | 4.3 |
| OVCAR-8 | | | |
| Ovary | 20.0 | Fetal Heart | 18.7 |
| Breast ca. MCF-7 | 5.6 | Heart Pool | 12.3 |
| Breast ca. MDA- | 41.5 | Lymph Node Pool | 18.9 |
| MB-231 | | | |
| Breast ca. BT 549 | 55.1 | Fetal Skeletal Muscle | 8.9 |
| Breast ca. T47D | 63.3 | Skeletal Muscle Pool | 23.2 |
| Breast ca. MDA-N | 23.5 | Spleen Pool | 10.8 |
| Breast Pool | 25.5 | Thymus Pool | 18.0 |
| Trachea | 13.6 | CNS cancer | 62.4 |
| | | (glio/astro) U87-MG | |
| Lung | 5.3 | CNS cancer | 12.5 |
| | | (glio/astro) U-118-MG | |
| Fetal Lung | 32.5 | CNS cancer | 8.1 |
| | | (neuro; met) SK-N-AS | |
| Lung ca. NCI-N417 | 6.2 | CNS cancer (astro) SF- | 17.3 |
| | | 539 | |
| Lung ca. LX-1 | 33.7 | CNS cancer (astro) | 27.2 |
| | | SNB-75 | |
| Lung ca. NCI-H146 | 10.0 | CNS cancer (glio) | 25.9 |
| | | SNB-19 | |
| Lung ca. SHP-77 | 39.2 | CNS cancer (glio) SF- | 54.0 |
| | | 295 | |
| Lung ca. A549 | 43.8 | Brain (Amygdala) Pool | 47.6 |
| Lung ca. NCI-H526 | 6.5 | Brain (cerebellum) | 90.1 |
| Lung ca. NCI-H23 | 44.8 | Brain (fetal) | 56.6 |
| Lung ca. NCI-H460 | 23.8 | Brain (Hippocampus) Pool | 45.7 |
| Lung ca. HOP-62 | 53.6 | Cerebral Cortex Pool | 60.3 |
| Lung ca. NCI-H522 | 100.0 | Brain (Substantia | 61.6 |
| | | nigra) Pool | |
| Liver | 4.4 | Brain (Thalamus) Pool | 75.8 |
| Fetal Liver | 17.0 | Brain (whole) | 63.3 |
| Liver ca. HepG2 | 33.9 | Spinal Cord Pool | 24.1 |
| Kidney Pool | 37.1 | Adrenal Gland | 23.5 |
| Fetal Kidney | 16.4 | Pituitary gland Pool | 3.9 |
| Renal ca. 786-0 | 14.8 | Salivary Gland | 9.9 |
| Renal ca. A498 | 7.5 | Thyroid (female) | 29.1 |
| Renal ca. ACHN | 26.2 | Pancreatic ca. | 39.8 |
| | | CAPAN2 | |
| Renal ca. UO-31 | 81.8 | Pancreas Pool | 49.3 |

TABLE AMD
Panel 4D

| | Rel. Exp. (%) | | Rel. Exp. (%) |
|---|---|---|---|
| | Ag3387, Run | | Ag3387, Run |
| Tissue Name | 165296475 | Tissue Name | 165296475 |
| Secondary Th1 act | 24.1 | HUVEC IL-1beta | 12.1 |
| Secondary Th2 act | 29.9 | HUVEC IFN gamma | 27.0 |
| Secondary Tr1 act | 26.2 | HUVEC TNF alpha + | 19.1 |
| | | IFN gamma | |
| Secondary Th1 rest | 18.9 | HUVEC TNF alpha + | 16.0 |
| | | IL4 | |
| Secondary Th2 rest | 22.8 | HUVEC IL-11 | 16.4 |
| Secondary Tr1 rest | 28.1 | Lung Microvascular EC | 36.6 |
| | | none | |
| Primary Th1 act | 12.4 | Lung Microvascular EC | 27.2 |
| | | TNF alpha + IL-1beta | |
| Primary Th2 act | 20.9 | Microvascular Dermal | 43.5 |
| | | EC none | |
| Primary Tr1 act | 25.7 | Microsvasular Dermal | 23.2 |
| | | EC TNF alpha + IL-1beta | |
| Primary Th1 rest | 46.0 | Bronchial epithelium | 18.0 |
| | | TNF alpha + IL1beta | |
| Primary Th2 rest | 36.1 | Small airway epithelium | 3.9 |
| | | none | |
| Primary Tr1 rest | 37.6 | Small airway epithelium | 33.2 |
| | | TNF alpha + IL-1beta | |
| CD45RA CD4 | 5.7 | Coronery artery SMC rest | 19.6 |
| lymphocyte act | | | |
| CD45RO CD4 | 19.9 | Coronery artery SMC | 12.7 |
| lymphocyte act | | TNF alpha + IL-1beta | |
| CD8 lymphocyte act | 16.2 | Astrocytes rest | 27.9 |
| Secondary CD8 | 11.3 | Astrocytes TNF alpha + | 21.8 |
| lymphocyte rest | | IL-1beta | |
| Secondary CD8 | 12.3 | KU-812 (Basophil) rest | 7.6 |
| lymphocyte act | | | |
| CD4 lymphocyte none | 5.7 | KU-812 (Basophil) | 22.1 |
| | | PMA/ionomycin | |
| 2ry Th1/Th2/Tr1_anti- | 12.2 | CCD1106 | 12.0 |
| CD95 CH11 | | (Keratinocytes) none | |
| LAK cells rest | 19.2 | CCD1106 | 19.3 |
| | | (Keratinocytes) | |
| | | TNF alpha + IL-1beta | |
| LAK cells IL-2 | 25.9 | Liver cirrhosis | 9.4 |
| LAK cells IL-2 + IL-12 | 10.2 | Lupus kidney | 3.2 |
| LAK cells IL-2 + IFN | 26.4 | NCI-H292 none | 62.0 |
| gamma | | | |
| LAK cells IL-2 + IL-18 | 21.2 | NCI-H292 IL-4 | 71.2 |
| LAK cells | 4.2 | NCI-H292 IL-9 | 52.5 |
| PMA/ionomycin | | | |
| NK Cells IL-2 rest | 21.2 | NCI-H292 IL-13 | 24.8 |
| Two Way MLR 3 day | 30.6 | NCI-H292 IFN gamma | 33.7 |
| Two Way MLR 5 day | 26.2 | HPAEC none | 27.7 |
| Two Way MLR 7 day | 16.8 | HPAEC TNF alpha + IL- | 12.6 |
| | | 1beta | |
| PBMC rest | 10.7 | Lung fibroblast none | 25.3 |
| PBMC PWM | 21.3 | Lung fibroblast TNF | 12.1 |
| | | alpha + IL-1beta | |
| PBMC PHA-L | 25.5 | Lung fibroblast IL-4 | 48.0 |
| Ramos (B cell) none | 18.4 | Lung fibroblast IL-9 | 34.6 |
| Ramos (B cell) | 68.3 | Lung fibroblast IL-13 | 51.1 |
| ionomycin | | | |
| B lymphocytes PWM | 54.0 | Lung fibroblast IFN | 63.3 |
| | | gamma | |
| B lymphocytes CD40L | 0.0 | Dermal fibroblast | 20.4 |
| and IL-4 | | CCD1070 rest | |
| EOL-1 dbcAMP | 18.4 | Dermal fibroblast | 74.2 |
| | | CCD1070 TNF alpha | |
| EOL-1 dbcAMP | 3.1 | Dermal fibroblast | 4.6 |
| PMA/ionomycin | | CCD1070 IL-1beta | |
| Dendritic cells none | 28.1 | Dermal fibroblast IFN | 10.1 |
| | | gamma | |
| Dendritic cells LPS | 29.7 | Dermal fibroblast IL-4 | 23.8 |
| Dendritic cells anti- | 19.6 | IBD Colitis 2 | 1.4 |
| CD40 | | | |
| Monocytes rest | 17.2 | IBD Crohn's | 2.4 |
| Monocytes LPS | 1.6 | Colon | 21.8 |
| Macrophages rest | 30.8 | Lung | 16.6 |
| Macrophages LPS | 17.4 | Thymus | 100.0 |
| HUVEC none | 22.8 | Kidney | 48.0 |
| HUVEC starved | 28.5 | | |

CNS_neurodegeneration_v1.0 Summary: Ag3387 This panel does not show differential expression of the CG58662-01 gene in Alzheimer's disease. However, this expression profile confirms the presence of this gene in the brain. Please see Panel 1.3D for discussion of utility of this gene in the central nervous system.

General_screening_panel_v1.4 Summary: Ag3387 Expression of the CG58662-01 gene is ubiquitous in this panel, with highest expression in a lung cancer cell line (CT=29.5). In addition, this gene is expressed at higher levels in kidney cancer cell lines when compared to normal kidney expression. Thus, expression of this gene could be used to differentiate these samples from other samples and as a marker for these cancers. Furthermore, therapeutic modulation of the expression of function of this gene may be effective in the treatment of lung and kidney cancer.

Among metabolic tissues this gene is expressed at moderate to low levels in adipose, adrenal gland, pancreas, pituitary, and adult and fetal skeletal muscle, heart and liver. This widespread expression among these tissues suggests that this gene plays a role in normal metabolic and neuroendocrine function and that disregulated expression of this gene may contribute to neuroendocrine diseases or metabolic disorders, such as obesity and diabetes.

In addition, this gene is expressed at moderate to low levels in all CNS regions examinded and may be a small molecule target for the treatment of neurologic diseases, such as Alzheimer's disease, Parkinson's disease, schizophrenia, multiple sclerosis, stroke and epilepsy.

Panel 4D Summary: Ag3387 The CG58662-01 gene is expressed at high to moderate levels in a wide range of cell types of significance in the immune response in health and disease, with highest expression in the thymus (CT=31). In addition, expression is seen in members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AN. CG59371-01: Novel Cytoplasmic Protein

Expression of gene CG59371-01 was assessed using the primer-probe set Ag3558, described in Table ANA. Results of the RTQ-PCR runs are shown in Tables ANB, ANC, AND and ANE.

Table ANA. Probe Name Ag3558

TABLE ANA
Probe Name Ag3558

| | | | | SEQ |
|---|---|---|---|---|
| | | | Start | ID |
| Primers | Sequences | Length | Position | NO: |
| Forward | 5′-cttgaggctgagaaggagaag-3′ | 21 | 208 | 517 |
| Probe | TET-5′-tgcttatcaactcacagagaaggaca-3′-TAMRA | 26 | 231 | 518 |
| Reverse | 5′-gttggtctctcagtcgctgta-3′ | 21 | 263 | 519 |

TABLE ANB
General_screening_panel_v1.4

| | Rel. Exp. (%) | | Rel. Exp. (%) |
|---|---|---|---|
| | Ag3558, Run | | Ag3558, Run |
| Tissue Name | 213391281 | Tissue Name | 213391281 |
| Adipose | 0.4 | Renal ca. TK-10 | 14.3 |
| Melanoma* Hs688(A).T | 1.9 | Bladder | 5.7 |
| Melanoma* | 2.2 | Gastric ca. (liver met.) | 6.4 |
| Hs688(B).T | | NCI-N87 | |
| Melanoma* M14 | 41.5 | Gastric ca. KATO III | 82.9 |
| Melanoma* LOXIMVI | 30.1 | Colon ca. SW-948 | 14.8 |
| Melanoma* SK-MEL-5 | 23.8 | Colon ca. SW480 | 81.2 |
| Squamous cell | 34.6 | Colon ca.* (SW480 | 24.0 |
| carcinoma SCC-4 | | met) SW620 | |
| Testis Pool | 7.6 | Colon ca. HT29 | 20.6 |
| Prostate ca.* (bone met) PC-3 | 9.7 | Colon ca. HCT-116 | 61.6 |
| Prostate Pool | 0.1 | Colon ca. CaCo-2 | 17.7 |
| Placenta | 0.3 | Colon cancer tissue | 7.7 |
| Uterus Pool | 0.1 | Colon ca. SW1116 | 6.9 |
| Ovarian ca. | 15.3 | Colon ca. Colo-205 | 3.2 |
| OVCAR-3 | | | |
| Ovarian ca. SK-OV-3 | 62.0 | Colon ca. SW-48 | 9.3 |
| Ovarian ca. OVCAR-4 | 27.5 | Colon Pool | 0.3 |
| Ovarian ca. | 14.4 | Small Intestine Pool | 0.1 |
| OVCAR-5 | | | |
| Ovarian ca. | 5.8 | Stomach Pool | 2.0 |
| IGROV-1 | | | |
| Ovarian ca. | 2.5 | Bone Marrow Pool | 0.3 |
| OVCAR-8 | | | |
| Ovary | 0.2 | Fetal Heart | 3.3 |
| Breast ca. MCF-7 | 15.5 | Heart Pool | 0.0 |
| Breast ca. MDA- | 100.0 | Lymph Node Pool | 0.5 |
| MB-231 | | | |
| Breast ca. BT 549 | 72.7 | Fetal Skeletal Muscle | 0.7 |
| Breast ca. T47D | 17.1 | Skeletal Muscle Pool | 0.0 |
| Breast ca. MDA-N | 13.5 | Spleen Pool | 0.8 |
| Breast Pool | 0.2 | Thymus Pool | 7.2 |
| Trachea | 0.4 | CNS cancer | 17.6 |
| | | (glio/astro) U87-MG | |
| Lung | 0.0 | CNS cancer | 61.6 |
| | | (glio/astro) U-118-MG | |
| Fetal Lung | 2.5 | CNS cancer | 14.9 |
| | | (neuro; met) SK-N-AS | |
| Lung ca. NCI-N417 | 5.4 | CNS cancer (astro) SF- | 21.6 |
| | | 539 | |
| Lung ca. LX-1 | 27.2 | CNS cancer (astro) | 30.4 |
| | | SNB-75 | |
| Lung ca. NCI-H146 | 15.2 | CNS cancer (glio) | 4.6 |
| | | SNB-19 | |
| Lung ca. SHP-77 | 42.0 | CNS cancer (glio) SF- | 0.8 |
| | | 295 | |
| Lung ca. A549 | 28.5 | Brain (Amygdala) Pool | 0.1 |
| Lung ca. NCI-H526 | 6.4 | Brain (cerebellum) | 0.0 |
| Lung ca. NCI-H23 | 21.3 | Brain (fetal) | 1.1 |
| Lung ca. NCI-H460 | 0.7 | Brain (Hippocampus) Pool | 0.2 |
| Lung ca. HOP-62 | 4.7 | Cerebral Cortex Pool | 0.1 |
| Lung ca. NCI-H522 | 23.8 | Brain (Substantia | 0.1 |
| | | nigra) Pool | |
| Liver | 0.0 | Brain (Thalamus) Pool | 0.0 |
| Fetal Liver | 0.8 | Brain (whole) | 0.0 |
| Liver ca. HepG2 | 7.7 | Spinal Cord Pool | 0.1 |
| Kidney Pool | 0.1 | Adrenal Gland | 0.2 |
| Fetal Kidney | 4.1 | Pituitary gland Pool | 0.0 |
| Renal ca. 786-0 | 52.5 | Salivary Gland | 0.0 |
| Renal ca. A498 | 6.5 | Thyroid (female) | 0.2 |
| Renal ca. ACHN | 14.0 | Pancreatic ca. CAPAN2 | 43.2 |
| Renal ca. UO-31 | 20.2 | Pancreas Pool | 0.9 |

TABLE ANC
General_screening_panel_v.1.5

| | Rel. Exp. (%) | | Rel. Exp. (%) |
|---|---|---|---|
| | Ag3558, Run | | Ag3558, Run |
| Tissue Name | 248592792 | Tissue Name | 248592792 |
| Adipose | 0.4 | Renal ca. TK-10 | 16.6 |
| Melanoma* Hs688(A).T | 2.0 | Bladder | 3.3 |
| Melanoma* | 3.1 | Gastric ca. (liver met.) | 6.7 |
| Hs688(B).T | | NCI-N87 | |
| Melanoma* M14 | 34.9 | Gastric ca. KATO III | 93.3 |
| Melanoma* LOXIMVI | 26.4 | Colon ca. SW-948 | 13.0 |
| Melanoma* SK-MEL-5 | 29.5 | Colon ca. SW480 | 76.3 |
| Squamous cell | 33.0 | Colon ca.* (SW480 | 25.5 |
| carcinoma SCC-4 | | met) SW620 | |
| Testis Pool | 7.2 | Colon ca. HT29 | 18.8 |
| Prostate ca.* (bone met) PC-3 | 10.3 | Colon ca. HCT-116 | 55.5 |
| Prostate Pool | 0.1 | Colon ca. CaCo-2 | 23.5 |
| Placenta | 0.1 | Colon cancer tissue | 5.0 |
| Uterus Pool | 0.2 | Colon ca. SW1116 | 4.6 |
| Ovarian ca. | 29.5 | Colon ca. Colo-205 | 5.6 |
| OVCAR-3 | | | |
| Ovarian ca. SK- | 40.6 | Colon ca. SW-48 | 6.8 |
| OV-3 | | | |
| Ovarian ca. | 29.5 | Colon Pool | 0.2 |
| OVCAR-4 | | | |
| Ovarian ca. | 11.7 | Small Intestine Pool | 0.2 |
| OVCAR-5 | | | |
| Ovarian ca. | 4.6 | Stomach Pool | 0.2 |
| IGROV-1 | | | |
| Ovarian ca. | 5.2 | Bone Marrow Pool | 0.2 |
| OVCAR-8 | | | |
| Ovary | 0.2 | Fetal Heart | 2.0 |
| Breast ca. MCF-7 | 18.0 | Heart Pool | 0.0 |
| Breast ca. MDA- | 85.3 | Lymph Node Pool | 0.5 |
| MB-231 | | | |
| Breast ca. BT 549 | 100.0 | Fetal Skeletal Muscle | 0.8 |
| Breast ca. T47D | 23.3 | Skeletal Muscle Pool | 0.0 |
| Breast ca. MDA-N | 20.2 | Spleen Pool | 1.0 |
| Breast Pool | 0.3 | Thymus Pool | 3.7 |
| Trachea | 0.4 | CNS cancer | 15.4 |
| | | (glio/astro) U87-MG | |
| Lung | 0.0 | CNS cancer | 64.6 |
| | | (glio/astro) U-118-MG | |
| Fetal Lung | 3.5 | CNS cancer | 24.7 |
| | | (neuro; met) SK-N-AS | |
| Lung ca. NCI-N417 | 5.0 | CNS cancer (astro) SF- | 21.6 |
| | | 539 | |
| Lung ca. LX-1 | 32.1 | CNS cancer (astro) | 30.4 |
| | | SNB-75 | |
| Lung ca. NCI-H146 | 13.6 | CNS cancer (glio) | 4.8 |
| | | SNB-19 | |
| Lung ca. SHP-77 | 34.9 | CNS cancer (glio) SF- | 3.3 |
| | | 295 | |
| Lung ca. A549 | 35.4 | Brain (Amygdala) Pool | 0.0 |
| Lung ca. NCI-H526 | 3.4 | Brain (cerebellum) | 0.0 |
| Lung ca. NCI-H23 | 15.9 | Brain (fetal) | 0.7 |
| Lung ca. NCI-H460 | 0.4 | Brain (Hippocampus) Pool | 0.0 |
| Lung ca. HOP-62 | 4.5 | Cerebral Cortex Pool | 0.0 |
| Lung ca. NCI-H522 | 25.5 | Brain (Substantia | 0.0 |
| | | nigra) Pool | |
| Liver | 0.0 | Brain (Thalamus) Pool | 0.0 |
| Fetal Liver | 1.3 | Brain (whole) | 0.0 |
| Liver ca. HepG2 | 6.6 | Spinal Cord Pool | 0.0 |
| Kidney Pool | 0.1 | Adrenal Gland | 0.1 |
| Fetal Kidney | 4.6 | Pituitary gland Pool | 0.0 |
| Renal ca. 786-0 | 44.1 | Salivary Gland | 0.0 |
| Renal ca. A498 | 4.2 | Thyroid (female) | 0.1 |
| Renal ca. ACHN | 15.2 | Pancreatic ca. | 48.3 |
| | | CAPAN2 | |
| Renal ca. UO-31 | 20.4 | Pancreas Pool | 0.5 |

TABLE AND
Panel 2.2

| | Rel. Exp. (%) | Rel. Exp. (%) | | Rel. Exp. (%) | Rel. Exp. (%) |
|---|---|---|---|---|---|
| | Ag3558, Run | Ag3558, Run | | Ag3558, Run | Ag3558, Run |
| Tissue Name | 173762113 | 174924057 | Tissue Name | 173762113 | 174924057 |
| Normal Colon | 12.5 | 5.6 | Kidney Margin | 3.3 | 1.1 |
| | | | (OD04348) | | |
| Colon cancer | 100.0 | 100.0 | Kidney | 5.6 | 8.9 |
| (OD06064) | | | malignant | | |
| | | | cancer | | |
| | | | (OD06204B) | | |
| Colon Margin | 27.5 | 17.7 | Kidney normal | 0.6 | 0.3 |
| (OD06064) | | | adjacent tissue | | |
| | | | (OD06204E) | | |
| Colon cancer | 3.1 | 4.8 | Kidney Cancer | 6.3 | 2.8 |
| (OD06159) | | | (OD04450-01) | | |
| Colon Margin | 5.6 | 7.3 | Kidney Margin | 0.6 | 0.0 |
| (OD06159) | | | (OD04450-03) | | |
| Colon cancer | 11.5 | 16.6 | Kidney Cancer | 0.0 | 0.0 |
| (OD06297-04) | | | 8120613 | | |
| Colon Margin | 12.5 | 7.8 | Kidney Margin | 0.3 | 0.3 |
| (OD06297-05) | | | 8120614 | | |
| CC Gr.2 ascend | 6.9 | 5.7 | Kidney Cancer | 0.2 | 1.9 |
| colon | | | 9010320 | | |
| (ODO3921) | | | | | |
| CC Margin | 6.8 | 6.4 | Kidney Margin | 2.1 | 2.3 |
| (ODO3921) | | | 9010321 | | |
| Colon cancer | 6.8 | 4.9 | Kidney Cancer | 0.8 | 1.7 |
| metastasis | | | 8120607 | | |
| (OD06104) | | | | | |
| Lung Margin | 17.9 | 12.6 | Kidney Margin | 0.3 | 0.0 |
| (OD06104) | | | 8120608 | | |
| Colon mets to | 15.1 | 23.3 | Normal Uterus | 1.6 | 0.5 |
| lung | | | | | |
| (OD04451-01) | | | | | |
| Lung Margin | 2.5 | 1.6 | Uterine Cancer | 3.5 | 3.6 |
| (OD04451-02) | | | 064011 | | |
| Normal Prostate | 2.1 | 0.0 | Normal | 0.3 | 0.0 |
| | | | Thyroid | | |
| Prostate Cancer | 0.0 | 0.2 | Thyroid Cancer | 1.1 | 0.7 |
| (OD04410) | | | 064010 | | |
| Prostate Margin | 0.4 | 0.3 | Thyroid Cancer | 1.2 | 1.0 |
| (OD04410) | | | A302152 | | |
| Normal Ovary | 2.7 | 0.4 | Thyroid Margin | 0.3 | 0.3 |
| | | | A302153 | | |
| Ovarian cancer | 30.1 | 32.8 | Normal Breast | 2.7 | 1.9 |
| (OD06283-03) | | | | | |
| Ovarian Margin | 1.4 | 1.3 | Breast Cancer | 7.4 | 8.5 |
| (OD06283-07) | | | (OD04566) | | |
| Ovarian Cancer | 7.0 | 1.9 | Breast Cancer | 4.3 | 6.5 |
| 064008 | | | 1024 | | |
| Ovarian cancer | 1.2 | 1.9 | Breast Cancer | 11.8 | 13.9 |
| (OD06145) | | | (OD04590-01) | | |
| Ovarian Margin | 0.9 | 0.6 | Breast Cancer | 8.0 | 6.8 |
| (OD06145) | | | Mets | | |
| | | | (OD04590-03) | | |
| Ovarian cancer | 28.1 | 30.4 | Breast Cancer | 7.9 | 11.0 |
| (OD06455-03) | | | Metastasis | | |
| | | | (OD04655-05) | | |
| Ovarian Margin | 0.7 | 0.6 | Breast Cancer | 5.7 | 5.8 |
| (OD06455-07) | | | 064006 | | |
| Normal Lung | 1.4 | 1.3 | Breast Cancer | 0.7 | 0.3 |
| | | | 9100266 | | |
| Invasive poor | 25.0 | 20.3 | Breast Margin | 1.8 | 1.1 |
| diff. lung adeno | | | 9100265 | | |
| (ODO4945-01 | | | | | |
| Lung Margin | 1.0 | 1.7 | Breast Cancer | 2.5 | 1.2 |
| (ODO4945-03) | | | A209073 | | |
| Lung Malignant | 6.3 | 6.4 | Breast Margin | 3.0 | 1.2 |
| Cancer | | | A2090734 | | |
| (OD03126) | | | | | |
| Lung Margin | 1.3 | 1.0 | Breast cancer | 15.7 | 24.7 |
| (OD03126) | | | (OD06083) | | |
| Lung Cancer | 13.5 | 10.3 | Breast cancer | 16.5 | 15.0 |
| (OD05014A) | | | node metastasis | | |
| | | | (OD06083) | | |
| Lung Margin | 3.1 | 4.8 | Normal Liver | 0.0 | 0.0 |
| (OD05014B) | | | | | |
| Lung cancer | 38.4 | 28.7 | Liver Cancer | 1.2 | 0.0 |
| (OD06081) | | | 1026 | | |
| Lung Margin | 1.2 | 1.1 | Liver Cancer | 4.1 | 1.3 |
| (OD06081) | | | 1025 | | |
| Lung Cancer | 7.4 | 3.8 | Liver Cancer | 0.9 | 0.9 |
| (OD04237-01) | | | 6004-T | | |
| Lung Margin | 0.8 | 1.9 | Liver Tissue | 2.2 | 1.4 |
| (OD04237-02) | | | 6004-N | | |
| Ocular | 0.2 | 0.6 | Liver Cancer | 0.8 | 1.0 |
| Melanoma | | | 6005-T | | |
| Metastasis | | | | | |
| Ocular | 0.0 | 0.0 | Liver Tissue | 1.3 | 0.3 |
| Melanoma | | | 6005-N | | |
| Margin (Liver) | | | | | |
| Melanoma | 13.2 | 12.5 | Liver Cancer | 1.6 | 2.0 |
| Metastasis | | | 064003 | | |
| Melanoma | 1.6 | 1.1 | Normal | 4.4 | 4.4 |
| Margin (Lung) | | | Bladder | | |
| Normal Kidney | 0.3 | 0.0 | Bladder Cancer | 4.6 | 1.9 |
| | | | 1023 | | |
| Kidney Ca, | 0.0 | 1.0 | Bladder Cancer | 23.0 | 15.4 |
| Nuclear grade 2 | | | A302173 | | |
| (OD04338) | | | | | |
| Kidney Margin | 0.8 | 0.3 | Normal | 6.5 | 9.5 |
| (OD04338) | | | Stomach | | |
| Kidney Ca | 0.6 | 2.6 | Gastric Cancer | 2.9 | 3.2 |
| Nuclear grade | | | 9060397 | | |
| 1/2 (OD04339) | | | | | |
| Kidney Margin | 0.8 | 0.6 | Stomach | 4.9 | 1.0 |
| (OD04339) | | | Margin | | |
| | | | 9060396 | | |
| Kidney Ca, | 0.6 | 1.2 | Gastric Cancer | 6.3 | 4.6 |
| Clear cell type | | | 9060395 | | |
| (OD04340) | | | | | |
| Kidney Margin | 0.7 | 1.4 | Stomach | 3.5 | 3.0 |
| (OD04340) | | | Margin | | |
| | | | 9060394 | | |
| Kidney Ca, | 36.9 | 31.0 | Gastric Cancer | 22.1 | 20.0 |
| Nuclear grade 3 | | | 064005 | | |
| (OD04348) | | | | | |

TABLE ANE
Panel 4D

| | Rel. Exp. (%) | | Rel. Exp. (%) |
|---|---|---|---|
| | Ag3558, Run | | Ag3558, Run |
| Tissue Name | 166488678 | Tissue Name | 166488678 |
| Secondary Th1 act | 17.9 | HUVEC IL-1beta | 10.3 |
| Secondary Th2 act | 12.4 | HUVEC IFN gamma | 12.2 |
| Secondary Tr1 act | 12.3 | HUVEC TNF alpha + | 12.8 |
| | | IFN gamma | |
| Secondary Th1 rest | 1.7 | HUVEC TNF alpha + | 14.9 |
| | | IL4 | |
| Secondary Th2 rest | 2.6 | HUVEC IL-11 | 8.6 |
| Secondary Tr1 rest | 2.5 | Lung Microvascular EC | 5.2 |
| | | none | |
| Primary Th1 act | 9.1 | Lung Microvascular EC | 4.8 |
| | | TNF alpha + IL-1beta | |
| Primary Th2 act | 11.6 | Microvascular Dermal | 19.5 |
| | | EC none | |
| Primary Tr1 act | 11.4 | Microsvasular Dermal | 8.5 |
| | | EC TNF alpha + IL-1beta | |
| Primary Th1 rest | 32.8 | Bronchial epithelium | 1.0 |
| | | TNF alpha + IL1beta | |
| Primary Th2 rest | 10.4 | Small airway epithelium | 0.5 |
| | | none | |
| Primary Tr1 rest | 13.4 | Small airway epithelium | 5.8 |
| | | TNF alpha + IL-1beta | |
| CD45RA CD4 | 10.7 | Coronery artery SMC rest | 2.3 |
| lymphocyte act | | | |
| CD45RO CD4 | 17.2 | Coronery artery SMC | 1.3 |
| lymphocyte act | | TNF alpha + IL-1beta | |
| CD8 lymphocyte act | 12.2 | Astrocytes rest | 1.4 |
| Secondary CD8 | 11.7 | Astrocytes TNF alpha + | 0.7 |
| lymphocyte rest | | IL-1beta | |
| Secondary CD8 | 10.4 | KU-812 (Basophil) rest | 3.1 |
| lymphocyte act | | | |
| CD4 lymphocyte none | 0.1 | KU-812 (Basophil) | 6.6 |
| | | PMA/ionomycin | |
| 2ry Th1/Th2/Tr1_anti- | 6.0 | CCD1106 | 11.3 |
| CD95 CH11 | | (Keratinocytes) none | |
| LAK cells rest | 1.8 | CCD1106 | 2.6 |
| | | (Keratinocytes) | |
| | | TNF alpha + IL-1beta | |
| LAK cells IL-2 | 16.3 | Liver cirrhosis | 0.3 |
| LAK cells IL-2 + IL-12 | 9.7 | Lupus Kidney | 0.0 |
| LAK cells IL-2 + IFN | 19.9 | NCI-H292 none | 12.2 |
| gamma | | | |
| LAK cells IL-2 + IL-18 | 16.4 | NCI-H292 IL-4 | 29.7 |
| LAK cells | 0.7 | NCI-H292 IL-9 | 24.3 |
| PMA/ionomycin | | | |
| NK Cells IL-2 rest | 9.9 | NCI-H292 IL-13 | 16.4 |
| Two Way MLR 3 day | 1.5 | NCI-H292 IFN gamma | 16.0 |
| Two Way MLR 5 day | 7.4 | HPAEC none | 8.9 |
| Two Way MLR 7 day | 6.1 | HPAEC TNF alpha + IL- | 5.5 |
| | | 1beta | |
| PBMC rest | 0.1 | Lung fibroblast none | 2.2 |
| PBMC PWM | 44.4 | Lung fibroblast TNF | 2.3 |
| | | alpha + IL-1beta | |
| PBMC PHA-L | 23.3 | Lung fibroblast IL-4 | 0.8 |
| Ramos (B cell) none | 13.4 | Lung fibroblast IL-9 | 2.3 |
| Ramos (B cell) | 47.0 | Lung fibroblast IL-13 | 0.5 |
| ionomycin | | | |
| B lymphocytes PWM | 79.0 | Lung fibroblast IFN | 0.5 |
| | | gamma | |
| B lymphocytes CD40L | 16.2 | Dermal fibroblast | 48.6 |
| and IL-4 | | CCD1070 rest | |
| EOL-1 dbcAMP | 6.3 | Dermal fibroblast | 100.0 |
| | | CCD1070 TNF alpha | |
| EOL-1 dbcAMP | 4.5 | Dermal fibroblast | 25.5 |
| PMA/ionomycin | | CCD1070 IL-1beta | |
| Dendritic cells none | 1.1 | Dermal fibroblast IFN | 14.0 |
| | | gamma | |
| Dendritic cells LPS | 0.1 | Dermal fibroblast IL-4 | 14.9 |
| Dendritic cells anti- | 0.1 | IBD Colitis 2 | 0.5 |
| CD40 | | | |
| Monocytes rest | 0.0 | IBD Crohn's | 0.2 |
| Monocytes LPS | 0.0 | Colon | 1.7 |
| Macropages rest | 3.0 | Lung | 1.2 |
| Macrophages LPS | 0.4 | Thymus | 0.0 |
| HUVEC none | 18.7 | Kidney | 11.4 |
| HUVEC starved | 0.0 | | |

CNS_neurodegeneration_v1.0 Summary: Ag3558 Expression of the CG59371-0l gene is low/undetectable in all samples on this panel (CTs>35). (Data not shown.)

General_screening_panel_v1.4 Summary: Ag3558 Highest expression of the CG59371-01 gene is seen in a breast cancer cell line (CT=23.4). Overall, expression of this gene is significantly higher in cancer cell lines and fetal derived tissues than in samples derived from normal adult tissues. There are significant levels of expression in clusters of cell lines derived from pancreatic, brain, colon, gastric, renal, lung, ovarian, breast and melanoma cancers. Thus, expression of this gene in could be used to differentiate between the cancer derived samples and fetal tissues from other samples on this panel and as a marker to detect the presence of cancer. Furthermore, the much higher levels of expression in proliferative tissue suggest that this gene may be involved in cell proliferation. Therefore, therapeutic modulation of the expression or function of this gene may be effective in the treatment of these cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is a novel protein phosphatase expressed at moderate to low levels in all regions of the CNS examined. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

General_screening_panel_v1.5 Summary: Ag3558 Results from this experiment are in excellent agreement with results from Panel 1.4. Please see that panel for discussion of utility of this gene in cancer, metabolic disorders and the central nervous system.

Panel 2.2 Summary: Ag3558 Two experiments with the same probe and primer produce results that are in excellent agreement, with highest expression of the CG59371-01 gene in colon cancer (CTs=30). Furthermore, expression is higher in kidney, lung, ovary and colon cancers when compared to normal adjacent tissue. In addition, significant expression is also seen in gastric, breast, and bladder cancer. Thus, expression of this gene in could be used to differentiate between the cancer derived samples and other samples on this panel and as a marker to detect the presence of cancer. Furthermore, therapeutic modulation of the expression or function of this gene may be effective in the treatment of these cancers.

Panel 4D Summary: Ag3558 The CG59371-01 gene is widely expressed among the samples on this panel, with highest expression in dermal fibroblasts treated with TNF-alpha. Significant levels of expression are also seen in treated and untreated samples from skin, lung, T-cells and B-cells. Therefore, modulation of the expression or activity of the protein encoded by this transcript through the application of antibodies or peptides therapeutics may be beneficial for the treatment of lung inflammatory diseases such as asthma, and chronic obstructive pulmonary diseases, inflammatory skin diseases such as psoriasis, atopic dermatitis, ulcerative dermatitis, and ulcerative colitis, autoimmune diseases such as Crohn's disease, lupus erythematosus, rheumatoid arthritis and osteoarthritis and in other diseases in which T cells and B cells are activated.

AO. CG59346-01: Cortactin-Binding Protein 1

Expression of gene CG59346-01 was assessed using the primer-probe set Ag3550, described in Table AOA. Results of the RTQ-PCR runs are shown in Tables AOB, AOC and AOD.

Table AOA. Probe Name Ag3550

TABLE AOA
Probe Name Ag3550

| | | | | SEQ ID |
|---|---|---|---|---|
| Primers | Sequence | Length | Start Position | NO: |
| Forward | 5′-gccaacagagatgaacaaagag-3′ | 22 | 3459 | 520 |
| Probe | TET-5′-accgcctctccttctcccgctct-3′-TAMRA | 23 | 3508 | 521 |
| Reverse | 5′-ttggaaggctaaagacatctga-3′ | 22 | 3532 | 522 |

TABLE AOB
CNS_neurodegeneration_v1.0

| | | | Rel. Exp. |
|---|---|---|---|
| | | | (%) |
| | Rel. Exp. (%) | | Ag3550, |
| | Ag3550, Run | | Run |
| Tissue Name | 210641081 | Tissue Name | 210641081 |
| AD 1 Hippo | 12.8 | Control (Path) 3 | 5.1 |
| | | Temporal Ctx | |
| AD 2 Hippo | 38.7 | Control (Path) 4 | 40.3 |
| | | Temporal Ctx | |
| AD 3 Hippo | 10.4 | AD 1 Occipital Ctx | 18.8 |
| AD 4 Hippo | 15.8 | AD 2 Occipital Ctx | 0.0 |
| | | (Missing) | |
| AD 5 Hippo | 79.6 | AD 3 Occipital Ctx | 7.2 |
| AD 6 Hippo | 49.3 | AD 4 Occipital Ctx | 25.9 |
| Control 2 Hippo | 37.4 | AD 5 Occipital Ctx | 37.6 |
| Control 4 Hippo | 10.3 | AD 6 Occipital Ctx | 19.6 |
| Control (Path) 3 | 9.6 | Control 1 Occipital | 2.1 |
| Hippo | | Ctx | |
| AD 1 Temporal | 15.7 | Control 2 Occipital | 56.6 |
| Ctx | | Ctx | |
| AD 2 Temporal | 37.1 | Control 3 Occipital | 26.8 |
| Ctx | | Ctx | |
| AD 3 Temporal | 8.6 | Control 4 Occipital | 5.0 |
| Ctx | | Ctx | |
| AD 4 Temporal | 30.6 | Control (Path) 1 | 93.3 |
| Ctx | | Occipital Ctx | |
| AD 5 Inf Temporal | 66.9 | Control (Path) 2 | 14.6 |
| Ctx | | Occipital Ctx | |
| AD 5 Sup | 38.7 | Control (Path) 3 | 2.6 |
| Temporal Ctx | | Occipital Ctx | |
| AD 6 Inf Temporal | 45.4 | Control (Path) 4 | 23.8 |
| Ctx | | Occipital Ctx | |
| AD 6 Sup | 53.2 | Control 1 Parietal | 8.7 |
| Temporal Ctx | | Ctx | |
| Control 1 | 7.3 | Control 2 Parietal | 48.0 |
| Temporal Ctx | | Ctx | |
| Control 2 | 36.6 | Control 3 Parietal | 17.2 |
| Temporal Ctx | | Ctx | |
| Control 3 | 29.7 | Control (Path) 1 | 84.1 |
| Temporal Ctx | | Parietal Ctx | |
| Control 3 | 14.6 | Control (Path) 2 | 28.5 |
| Temporal Ctx | | Parietal Ctx | |
| Control (Path) 1 | 100.0 | Control (Path) 3 | 4.6 |
| Temporal Ctx | | Parietal Ctx | |
| Control (Path) 2 | 65.5 | Control (Path) 4 | 56.6 |
| Temporal Ctx | | Parietal Ctx | |

TABLE AOC
General_screening_panel_v1.4

| | Rel. Exp. (%) | | Rel. Exp. (%) |
|---|---|---|---|
| | Ag3550, Run | | Ag3550, Run |
| Tissue Name | 217048931 | Tissue Name | 217048931 |
| Adipose | 0.5 | Renal ca. TK-10 | 27.7 |
| Melanoma* Hs688(A).T | 1.4 | Bladder | 13.7 |
| Melanoma* | 1.6 | Gastric ca. (liver met.) | 25.0 |
| Hs688(B).T | | NCI-N87 | |
| Melanoma* M14 | 0.0 | Gastric ca. KATO III | 24.5 |
| Melanoma* | 0.0 | Colon ca. SW-948 | 1.4 |
| LOXIMVI | | | |
| Melanoma* SK-MEL-5 | 0.8 | Colon ca. SW480 | 8.8 |
| Squamous cell | 2.1 | Colon ca.* (SW480 | 7.4 |
| carcinoma SCC-4 | | met) SW620 | |
| Testis Pool | 2.0 | Colon ca. HT29 | 2.0 |
| Prostate ca.* (bone | 15.2 | Colon ca. HCT-116 | 7.1 |
| met) PC-3 | | | |
| Prostate Pool | 6.7 | Colon ca. CaCo-2 | 92.0 |
| Placenta | 18.9 | Colon cancer tissue | 6.0 |
| Uterus Pool | 0.0 | Colon ca. SW1116 | 1.8 |
| Ovarian ca. | 7.6 | Colon ca. Colo-205 | 3.0 |
| OVCAR-3 | | | |
| Ovarian ca. SK- | 14.9 | Colon ca. SW-48 | 3.1 |
| OV-3 | | | |
| Ovarian ca. | 3.4 | Colon Pool | 2.2 |
| OVCAR-4 | | | |
| Ovarian ca. | 24.5 | Small Intestine Pool | 5.0 |
| OVCAR-5 | | | |
| Ovarian ca. | 2.1 | Stomach Pool | 6.9 |
| IGROV-1 | | | |
| Ovarian ca. | 2.4 | Bone Marrow Pool | 0.2 |
| OVCAR-8 | | | |
| Ovary | 1.4 | Fetal Heart | 0.1 |
| Breast ca. MCF-7 | 34.6 | Heart Pool | 0.1 |
| Breast ca. MDA- | 8.2 | Lymph Node Pool | 2.8 |
| MB-231 | | | |
| Breast ca. BT 549 | 0.2 | Fetal Skeletal Muscle | 0.2 |
| Breast ca. T47D | 57.4 | Skeletal Muscle Pool | 0.0 |
| Breast ca. MDA-N | 0.0 | Spleen Pool | 1.8 |
| Breast Pool | 4.6 | Thymus Pool | 7.2 |
| Trachea | 14.1 | CNS cancer | 0.1 |
| | | (glio/astro) U87-MG | |
| Lung | 0.0 | CNS cancer | 0.1 |
| | | (glio/astro) U118-MG | |
| Fetal Lung | 19.5 | CNS cancer | 0.0 |
| | | (neuro; met) SK-N-AS | |
| Lung ca. NCI-N417 | 1.5 | CNS cancer (astro) SF- | 0.0 |
| | | 539 | |
| Lung ca. LX-1 | 24.7 | CNS cancer (astro) | 0.0 |
| | | SNB-75 | |
| Lung ca. NCI-H146 | 7.4 | CNS cancer (glio) | 2.1 |
| | | SNB-19 | |
| Lung ca. SHP-77 | 0.2 | CNS cancer (glio) SF- | 0.1 |
| | | 295 | |
| Lung ca. A549 | 26.1 | Brain (Amygdala) Pool | 22.1 |
| Lung ca. NCI-H526 | 13.9 | Brain (cerebellum) | 63.3 |
| Lung ca. NCI-H23 | 6.6 | Brain (fetal) | 100.0 |
| Lung ca. NCI-H460 | 11.1 | Brain (Hippocampus) Pool | 28.1 |
| Lung ca. HOP-62 | 0.2 | Cerebral Cortex Pool | 34.2 |
| Lung ca. NCI-H522 | 0.5 | Brain (Substantia | 26.2 |
| | | nigra) Pool | |
| Liver | 3.6 | Brain (Thalamus) Pool | 37.9 |
| Fetal Liver | 19.1 | Brain (whole) | 57.8 |
| Liver ca. HepG2 | 26.4 | Spinal Cord Pool | 2.8 |
| Kidney Pool | 0.3 | Adrenal Gland | 2.6 |
| Fetal Kidney | 11.7 | Pituitary gland Pool | 3.6 |
| Renal ca. 786-0 | 23.3 | Salivary Gland | 25.5 |
| Renal ca. A498 | 11.0 | Thyroid (female) | 6.9 |
| Renal ca. ACHN | 27.7 | Pancreatic ca. | 15.3 |
| | | CAPAN2 | |
| Renal ca. UO-31 | 12.7 | Pancreas Pool | 12.9 |

TABLE AOD
Panel 4D

| | Rel. Exp. (%) | | Rel. Exp. (%) |
|---|---|---|---|
| | Ag3550, Run | | Ag3550, Run |
| Tissue Name | 166453850 | Tissue Name | 166453850 |
| Secondary Th1 act | 0.2 | HUVEC IL-1beta | 0.0 |
| Secondary Th2 act | 0.1 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 9.5 |
| | | TNF alpha + IL1beta | |
| Primary Th2 rest | 0.0 | Small airway epithelium | 8.1 |
| | | none | |
| Primary Tr1 rest | 0.0 | Small airway epithelium | 27.2 |
| | | TNF alpha + IL-1beta | |
| CD45RA CD4 | 1.4 | Coronery artery SMC rest | 0.7 |
| lymphocyte act | | | |
| CD45RO CD4 | 0.0 | Coronery artery SMC | 1.0 |
| lymphocyte act | | TNF alpha + IL-1beta | |
| CD8 lymphocyte act | 0.0 | Astrocytes rest | 5.2 |
| Secondary CD8 | 0.0 | Astrocytes TNF alpha + | 5.9 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.1 |
| | | PMA/ionomycin | |
| 2ry Th1/Th2/Tr1_anti- | 0.0 | CCD1106 | 10.5 |
| CD95 CH11 | | (Keratinocytes) none | |
| LAK cells rest | 0.0 | CCD1106 | 23.0 |
| | | (Keratinocytes) | |
| | | TNF alpha + IL-1beta | |
| LAK cells IL-2 | 0.0 | Liver cirrhosis | 22.8 |
| LAK cells IL-2 + IL-12 | 0.1 | Lupus kidney | 22.5 |
| LAK cells IL-2 + IFN | 0.0 | NCI-H292 none | 66.0 |
| gamma | | | |
| LAK cells IL-2 + IL-18 | 0.0 | NCI-H292 IL-4 | 81.8 |
| LAK cells | 0.0 | NCI-H292 IL-9 | 69.7 |
| PMA/ionomycin | | | |
| NK Cells IL-2 rest | 0.0 | NCI-H292 IL-13 | 58.6 |
| Two Way MLR 3 day | 0.0 | NCI-H292 IFN gamma | 61.1 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 57.4 |
| PBMC PWM | 0.0 | Lung fibroblast TNF | 45.7 |
| | | alpha + IL-1beta | |
| PBMC PHA-L | 0.0 | Lung fibroblast IL-4 | 36.6 |
| Ramos (B cell) none | 0.0 | Lung fibroblast IL-9 | 33.9 |
| Ramos (B cell) | 0.0 | Lung fibroblast IL-13 | 17.2 |
| ionomycin | | | |
| B lymphocytes PWM | 2.4 | Lung fibroblast IFN | 34.9 |
| | | gamma | |
| B lymphocytes CD40L | 11.3 | Dermal fibroblast | 28.5 |
| and IL-4 | | CCD1070 rest | |
| EOL-1 dbcAMP | 0.0 | Dermal fibroblast | 11.3 |
| | | CCD1070 TNF alpha | |
| EOL-1 dbcAMP | 0.0 | Dermal fibroblast | 4.8 |
| PMA/ionomycin | | CCD1070 IL-1beta | |
| Dendritic cells none | 0.0 | Dermal fibroblast IFN | 0.3 |
| | | gamma | |
| Dendritic cells LPS | 0.0 | Dermal fibroblast IL-4 | 0.0 |
| Dendritic cells anti- | 0.3 | IBD Colitis 2 | 6.9 |
| CD40 | | | |
| Monocytes rest | 36.3 | IBD Crohn's | 2.0 |
| Monocytes LPS | 0.0 | Colon | 36.6 |
| Macrophages rest | 0.0 | Lung | 14.3 |
| Macrophages LPS | 0.0 | Thymus | 100.0 |
| HUVEC none | 0.0 | Kidney | 0.1 |
| HUVEC starved | 0.0 | | |

CNS_neurodegeneration_v1.0 Summary: Ag3550 This panel does not show differential expression of the CG59346-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3550 Highest expression of the CG59346-01 gene is seen in the brain. Expression of this gene is seen at high levels in the cerebellum, cerebral cortex, and thalamus and at moderate levels in the amygdala, hippocampus, and thalamus. This CG59346-01 gene encodes a homologue of Proline-rich synapse-associated protein-1/cortactin binding protein 1 (ProSAP1/CortBP1). ProSAP1 is PDZ-domain protein highly enriched in the postsynaptic density (PSD) and involved in in the assembly of the PSD during neuronal differentiation that may function with contactin, in the recruitment and activation of neural intracellular signaling pathways. Therefore, this gene may play a role in central nervous system disorders such as Alzheimer's disease, Parkinson's disease, epilepsy, multiple sclerosis, schizophrenia and depression.

In addition, moderate levels of expression are seen in colon, gastric, renal, pancreatic, lung, ovarian, breast and prostate cancer cell lines. Thus, expression of this gene could be used to detect the presence of cancer. Furthermore, therapeutic modulation of the expression or function of this gene may be effective in the treatment of these cancers.

Among tissues with metabolic function, this gene is expressed at moderate to low levels in pituitary, adipose, adrenal gland, pancreas, thyroid, and adult and fetal and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at higher levels in fetal lung and kidney (CTs=29) when compared to expression in adult lung and kidney (CTs=35-40). Thus, expression of this gene could be used to differentiate between the two sources of lung and kidney tissue.

### REFERENCES

1. Peles E, Nativ M, Lustig M, Grumet M, Schilling J, Martinez R, Plowman G D, Schlessinger J. Identification of a novel contactin-associated transmembrane receptor with multiple domains implicated in protein-protein interactions. EMBO J. Mar. 3, 1997;16(5):978-88.

2. Boeckers T M, Kreutz M R, Winter C, Zuschratter W, Smalla K H, Sanmarti-Vila L, Wex H, Langnaese K, Bockmann J, Garner C C, Gundelfinger E D. (1999) Proline-rich synapse-associated protein-1/cortactin binding protein 1 (ProSAP1/CortBP1) is a PDZ-domain protein highly enriched in the postsynaptic density. J Neurosci Aug. 1, 1999;19(15):6506-18.

Panel 4D Summary: Ag3550 Highest expression of the CG59346-01 gene is seen in thymus (CT=27). In addition, significant levels of expression are seen in IL-4, IL-9, IL-13 and IFN gamma activated-NCI-H292 mucoepidermoid cells as well as untreated NCI-H292 cells. Moderate/low expression is also detected in IL-4, IL-9, IL-13 and IFN gamma activated lung fibroblasts, small airway epithelium (treated and untreated), and treated bronchial epithelium. The expression of this gene in cells derived from or within the lung suggests, that this gene may be involved in normal conditions as well as pathological and inflammatory lung disorders that include chronic obstructive pulmonary disease, asthma, allergy and emphysema.

In addition, significant levels of expression are seen in treated and untreated dermal fibroblasts and keratinocytes, suggesting that modulation of the expression or function of this gene may also reduce symtptoms in inflammatory skin diseases such as psoriasis, atopic dermatitis, and ulcerative dermatitis.

AP. CG57814-01 and CG57814-02: Basic I 19 Protein

Expression of gene CG57814-01 and varian CG57814-02 was assessed using the primer-p robe set Ag791, described in Table APA.

Table APA. Probe Name Ag791

TABLE APA
Probe Name Ag791

| | | | Start | SEQ ID |
|---|---|---|---|---|
| Primers | Sequences | Length | Position | NO: |
| Forward | 5′-aaatgtgatgaccaaggttctg-3′ | 22 | 1290 | 523 |
| Probe | TET-5′-agcacacattatccagcgaaagcatg-3′-TAMRA | 26 | 1319 | 524 |
| Reverse | 5′-tgtcaaagaaacccttgttgtc-3′ | 22 | 1368 | 525 |

Panel 1.2 Summary: Ag791 Expression of the CG57814-01 gene is low/undetectable in all samples on this panel (CTs>35). (Data not shown.)

AQ. CG59327-01: Monocarboxylate Transporter 1 Like Protein

Expression of gene CG59327-01 was assessed using the primer-probe set Ag3548, described in Table AQA. Results of the RTQ-PCR runs are shown in Tables AQB and AQC.

Table AOA. Probe Name Ag3548

TABLE AQA
Probe Name Ag3548

| | | | Start | SEQ ID |
|---|---|---|---|---|
| Primers | Sequences | Length | Position | NO: |
| Forward | 5′-atttgcatacagcagctttgtc-3′ | 22 | 517 | 526 |
| Probe | TET-5′-ttcatctcccagaaatcgtcaatttg-3′-TAMRA | 26 | 549 | 527 |
| Reverse | 5′-accttcgtttgctccaataagt-3′ | 22 | 579 | 528 |

| | Rel. Exp. (%) | | Rel. Exp. (%) |
|---|---|---|---|
| | Ag3548, Run | | Ag3548, Run |
| Tissue Name | 217048438 | Tissue Name | 217048428 |
| Adipose | 0.0 | Renal ca. TK-10 | 3.6 |
| Melanoma* Hs688(A).T | 0.0 | Bladder | 0.0 |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK-MEL-5 | 0.0 | Colon ca. SW480 | 1.3 |
| Squamous cell | 0.0 | Colon ca.* (SW480 | 0.0 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 1.2 | Colon ca. SW1116 | 0.0 |
| Ovarian ca. | 0.0 | Colon ca. Colo-205 | 0.0 |
| OVCAR-3 | | | |
| Ovarian ca. SK-OV-3 | 0.0 | Colon ca. SW-48 | 0.0 |
| Ovarian ca. | 0.0 | Colon Pool | 2.2 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 1.9 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer | 0.0 |
| | | (glio/astro) U87-MG | |
| Lung | 2.4 | CNS cancer | 6.5 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 6.4 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 0.0 |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) Pool | 0.0 |
| Lung ca. HOP-62 | 0.0 | Cerebral Cortex Pool | 0.0 |
| Lung ca. NCI-H522 | 3.1 | Brain (Substantia | 0.0 |
| | | nigra) Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 3.4 | Pituitary gland Pool | 0.0 |
| Renal ca. 786-0 | 0.0 | Salivary Gland | 0.0 |
| Renal ca. A498 | 100.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

TABLE AQC
Panel 4D

| | Rel. Exp. (%) | | Rel. Exp. (%) |
|---|---|---|---|
| | Ag3548, Run | | Ag3548, Run |
| Tissue Name | 166453848 | Tissue Name | 166453848 |
| SecondaryTh1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.5 |
| Secondary Tr1 rest | 0.0 | Lung Microvascular EC | 0.0 |
| | | none | |
| PrimaryTh1 act | 0.0 | Lung Microvascular EC | 0.0 |
| | | TNF alpha + IL-1beta | |
| Primary Th2 act | 0.0 | Microvascular Dermal | 100.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.2 |
| | | TNF alpha + IL-1beta | |
| Primary Th2 rest | 0.0 | Small airway epithelium | 1.2 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.3 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.3 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 6.5 |
| LAK cells IL-2 + IL-12 | 0.0 | Lupus kidney | 0.7 |
| LAK cells IL-2 + IFN | 0.0 | NCI-H292 none | 1.8 |
| gamma | | | |
| LAK cells IL-2 + IL-18 | 0.0 | NCI-H292 IL-4 | 1.7 |
| LAK cells | 0.0 | NCI-H292 IL-9 | 0.2 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.5 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 2.0 |
| CD40 | | | |
| Monocytes rest | 0.0 | IBD Crohn's | 3.5 |
| Monocytes LPS | 0.0 | Colon | 1.0 |
| Macrophages rest | 0.0 | Lung | 2.3 |
| Macrophages LPS | 0.0 | Thymus | 0.7 |
| HUVEC none | 0.0 | Kidney | 0.0 |
| HUVEC starved | 0.4 | | |

CNS_neurodegeneration_v1.0 Summary: Ag3548 Expression of the CG59327-01 gene is low/undetectable in all the samples on the panel (CTs>35). (Data not shown.)

General_screening_panel_v1.4 Summary: Ag3548 Significant expression of the CG59327-01 gene is restricted to a sample derived from a kidney cancer cell line (CT=33.34). Thus, expression of this gene could be used to differentiate between this sample and other samples on this panel and as a marker to detect the presence of kidney cancer. Furthermore, therapeutic modulation of the expression or function of this gene may be effective in the treatment of kidney cancer.

Panel 4D Summary: Ag3548 Significant expression of the CG59327-01 gene is restricted to a samples derived from untreated microvascular dermal endothelial cells (CT=30.3). Thus, expression of this gene could be used as a marker of these cells.

AR. CG59494-01: Myelin P2

Expression of gene CG59494-01, which represents a full length physical clone, was assessed using the primer-probe set Ag3206, described in Table ARA. Results of the RTQ-PCR runs are shown in Tables ARB and ARC.

Table ARA. Probe Name Ag3206

TABLE ARA
Probe Name Ag3206

| | | | Start | SEQ ID |
|---|---|---|---|---|
| Primers | Sequences | Length | Position | NO: |
| Forward | 5′-agtgttgatgggaaaatgatga-3′ | 22 | 160 | 455 |
| Probe | TET-5′-ccataagaacagaaagttctttccaggaca-3′-TAMRA | 30 | 182 | 758 |
| Reverse | 5′-ccccagcttgaaggagatc-3′ | 19 | 216 | 759 |

TABLE ARB
Panel 1.3D

| | Rel. Exp. (%) | | Rel. Exp. (%) |
|---|---|---|---|
| | Ag3206, Run | | Ag3206, Run |
| Tissue Name | 165527079 | Tissue Name | 165527079 |
| Liver adenocarcinoma | 0.0 | Kidney (fetal) | 10.5 |
| Pancreas | 0.0 | Renal ca. 786-0 | 0.0 |
| Pancreatic ca. CAPAN 2 | 17.8 | Renal ca. A498 | 15.6 |
| Adrenal gland | 0.0 | Renal ca. RXF 393 | 0.0 |
| Thyroid | 0.0 | Renal ca. ACHN | 14.8 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 9.9 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 4.5 |
| Brain (thalamus) | 0.0 | Lung ca. (small cell) | 0.0 |
| | | NCI-H69 | |
| Cerebral Cortex | 0.0 | Lung ca. (s.cell var.) | 18.0 |
| | | SHP-77 | |
| Spinal cord | 33.4 | Lung ca. (large | 41.2 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s.cell) | 0.0 |
| | | NCI-H23 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 11.7 | Lung ca. (squam.) | 0.0 |
| | | NCI-H596 | |
| glioma SNB-19 | 0.0 | Mammary gland | 14.4 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (fetal) | 0.0 | Breast ca.* (pl.ef) | 0.0 |
| | | T47D | |
| Heart | 15.5 | Breast ca. BT-549 | 0.0 |
| Skeletal muscle (fetal) | 0.0 | Breast ca. MDA-N | 0.0 |
| Skeletal muscle | 0.0 | Ovary | 0.0 |
| Bone marrow | 0.0 | Ovarian ca. | 14.0 |
| | | OVCAR-3 | |
| Thymus | 0.0 | Ovarian ca. | 0.0 |
| | | OVCAR-4 | |
| Spleen | 0.0 | Ovarian ca. | 0.0 |
| | | OVCAR-5 | |
| Lymph node | 0.0 | Ovarian ca. | 0.0 |
| | | OVCAR-8 | |
| Colorectal | 0.0 | Ovarian ca. IGROV-1 | 11.6 |
| Stomach | 0.0 | Ovarian ca.* | 0.0 |
| | | (ascites) SK-OV-3 | |
| Small intestine | 0.0 | Uterus | 0.0 |
| Colon ca. SW480 | 0.0 | Placenta | 0.0 |
| Colon ca.* | 0.0 | Prostate | 0.0 |
| SW620(SW480 met) | | | |
| Colon ca. HT29 | 0.0 | Prostate ca.* (bone | 100.0 |
| | | met)PC-3 | |
| Colon ca. HCT-116 | 0.0 | Testis | 27.5 |
| Colon ca. CaCo-2 | 42.0 | Melanoma | 0.0 |
| | | Hs688(A).T | |
| Colon ca. | 0.0 | Melanoma* (met) | 0.0 |
| tissue(ODO3866) | | Hs688(B).T | |
| Colon ca. HCC-2998 | 0.0 | Melanoma UACC-62 | 0.0 |
| Gastric ca.* (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 0.0 |

TABLE ARC
Panel 4D

| | Rel. Exp. (%) | | Rel. Exp. (%) |
|---|---|---|---|
| | Ag3206, Run | | Ag3206, Run |
| Tissue Name | 164531735 | Tissue Name | 164531735 |
| Secondary Th1 act | 0.0 | HUVEC IL-1beta | 0.0 |
| Secondary Th2 act | 11.9 | HUVEC IFN gamma | 0.0 |
| Secondary Tr1 act | 0.0 | HUVEC TNF alpha + | 12.6 |
| | | IFN gamma | |
| Secondary Th1 rest | 11.9 | HUVEC TNF alpha + | 15.9 |
| | | IL4 | |
| Secondary Th2 rest | 0.0 | HUVEC IL-11 | 0.0 |
| Secondary Tr1 rest | 0.0 | Lung Microvascular EC | 75.8 |
| | | none | |
| Primary Th1 act | 0.0 | Lung Microvascular EC | 100.0 |
| | | TNF alpha + IL-1beta | |
| Primary Th2 act | 0.0 | Microvascular Dermal | 72.2 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 29.7 |
| LAK cells IL-2 + IL-12 | 0.0 | Lupus kidney | 0.0 |
| LAK cells IL-2 + IFN | 0.0 | NCI-H292 none | 97.3 |
| gamma | | | |
| LAK cells IL-2 + IL-18 | 0.0 | NCI-H292 IL-4 | 0.0 |
| LAK cells | 0.0 | NCI-H292 IL-9 | 43.8 |
| PMA/ionomycin | | | |
| NK Cells IL-2 rest | 7.8 | NCI-H292 IL-13 | 24.0 |
| Two Way MLR 3 day | 0.0 | NCI-H292 IFN gamma | 12.7 |
| Two Way MLR 5 day | 0.0 | HPAEC none | 14.3 |
| Two Way MLR 7 day | 0.0 | HPAEC TNF alpha + IL- | 33.2 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 16.2 |
| Ramos (B cell) | 0.0 | Lung fibroblast IL-13 | 15.9 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 15.0 |
| Dendritic cells anti- | 0.0 | IBD Colitis 2 | 27.4 |
| CD40 | | | |
| Monocytes rest | 0.0 | IBD Crohn's | 0.0 |
| Monocytes LPS | 0.0 | Colon | 6.7 |
| Macrophages rest | 0.0 | Lung | 0.0 |
| Macrophages LPS | 0.0 | Thymus | 0.0 |
| HUVEC none | 27.7 | Kidney | 0.0 |
| HUVEC starved | 20.0 | | |

Panel 1.3D Summary: Ag3206 Expression of the CG59494-01 gene is restricted to a sample derived from a prostate cancer cell line (CT=34.9). Thus, expression of this gene could be used to differentiate between this sample and other samples on this panel and as a marker to detect the presence of prostate cancer. Furthermore, therapeutic modulation of the expression or function of this gene may be effective in the treatment of prostate cancer.

Panel 4D Summary: Ag3206 Expression of the CG59494-01 gene is primarily restricted to a cluster of samples derived from microvasculature of the lung and the dermis suggesting a role for this gene in the maintenance of the integrity of the microvasculature. Therefore, therapeutics designed for this putative protein could be beneficial for the treatment of diseases associated with damaged microvasculature including heart diseases or inflammatory diseases, such as psoriasis, asthma, and chronic obstructive pulmonary diseases.

AS. CG59432-01 and CG59432-02: Chloride Channel

Expression of gene CG59432-01 and CG59432-02 was assessed using the primer-110 probe set Ag5938, described in Table ASA. Results of the RTQ-PCR runs are shown in Tables ASB and ASC. Please note that CG59432-02 represents a full-length physical clone of CG59432-01 gene, validating the prediction of the gene sequence.

Table ASA. Probe Name Ag5938

TABLE ASA
Probe Name Ag5938

| | | | | SEQ ID |
|---|---|---|---|---|
| Primers | Sequences | Length | Start Position | NO: |
| Forward | 5′-ttgtgtcagtctataccattaa-3′ | 22 | 626 | 529 |
| Probe | TET-5′-accagcttggcctctgtccagt-3′-TAMRA | 22 | 658 | 530 |
| Reverse | 5′-tcctggagttcagagtatatct-3′ | 22 | 710 | 531 |

TABLE ASB
General_screening_panel_v1.5

| | Rel. Exp. (%) | | Rel. Exp. (%) |
|---|---|---|---|
| | Ag5938, Run | | Ag5938, Run |
| Tissue Name | 248102142 | Tissue Name | 248102142 |
| Adipose | 6.3 | Renal ca. TK-10 | 2.7 |
| Melanoma* Hs688(A).T | 0.0 | Bladder | 17.6 |
| Melanoma* | 0.0 | Gastric ca. (liver met.) | 100.0 |
| Hs688(B).T | | NCI-N87 | |
| Melanoma* M14 | 0.0 | Gastric ca. KATO III | 8.2 |
| Melanoma* | 0.0 | Colon ca. SW-948 | 0.0 |
| LOXIMVI | | | |
| Melanoma* SK-MEL-5 | 0.0 | Colon ca. SW480 | 7.5 |
| Squamous cell | 21.5 | Colon ca.* (SW480 | 0.6 |
| carcinoma SCC-4 | | met) SW620 | |
| Testis Pool | 21.0 | Colon ca. HT29 | 7.1 |
| Prostate ca.* (bone | 0.0 | Colon ca. HCT-116 | 11.0 |
| met) PC-3 | | | |
| Prostate Pool | 6.9 | Colon ca. CaCo-2 | 25.2 |
| Placenta | 0.0 | Colon cancer tissue | 4.7 |
| Uterus Pool | 1.5 | Colon ca. SW1116 | 2.7 |
| Ovarian ca. | 20.2 | Colon ca. Colo-205 | 0.0 |
| OVCAR-3 | | | |
| Ovarian ca. SK-OV-3 | 0.0 | Colon ca. SW-48 | 0.0 |
| Ovarian ca. | 0.0 | Colon Pool | 5.7 |
| OVCAR-4 | | | |
| Ovarian ca. | 24.7 | Small Intestine Pool | 7.6 |
| OVCAR-5 | | | |
| Ovarian ca. | 0.0 | Stomach Pool | 3.2 |
| IGROV-1 | | | |
| Ovarian ca. | 2.8 | Bone Marrow Pool | 6.6 |
| OVCAR-8 | | | |
| Ovary | 0.0 | Fetal Heart | 1.0 |
| Breast ca. MCF-7 | 4.2 | Heart Pool | 4.3 |
| Breast ca. MDA- | 0.0 | Lymph Node Pool | 3.8 |
| MB-231 | | | |
| Breast ca. BT 549 | 0.0 | Fetal Skeletal Muscle | 59.9 |
| Breast ca. T47D | 0.0 | Skeletal Muscle Pool | 93.3 |
| Breast ca. MDA-N | 0.0 | Spleen Pool | 3.2 |
| Breast Pool | 10.4 | Thymus Pool | 3.6 |
| Trachea | 15.1 | CNS cancer | 0.0 |
| | | (glio/astro) U87-MG | |
| Lung | 0.6 | CNS cancer | 3.1 |
| | | (glio/astro) U-118-MG | |
| Fetal Lung | 31.6 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 3.2 | CNS cancer (glio) | 3.6 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 47.3 |
| Lung ca. NCI-H526 | 32.8 | Brain (cerebellum) | 15.3 |
| Lung ca. NCI-H23 | 0.0 | Brain (fetal) | 1.8 |
| Lung ca. NCI-H460 | 0.0 | Brain (Hippocampus) Pool | 15.8 |
| Lung ca. HOP-62 | 0.0 | Cerebral Cortex Pool | 27.0 |
| Lung ca. NCI-H522 | 0.0 | Brain (Substantia | 25.2 |
| | | nigra) Pool | |
| Liver | 0.0 | Brain (Thalamus) Pool | 29.1 |
| Fetal Liver | 7.0 | Brain (whole) | 9.5 |
| Liver ca. HepG2 | 0.0 | Spinal Cord Pool | 11.0 |
| Kidney Pool | 6.8 | Adrenal Gland | 8.4 |
| Fetal Kidney | 17.1 | Pituitary gland Pool | 6.3 |
| Renal ca. 786-0 | 0.0 | Salivary Gland | 4.8 |
| Renal ca. A498 | 0.0 | Thyroid (female) | 0.0 |
| Renal ca. ACHN | 0.0 | Pancreatic ca. | 1.4 |
| | | CAPAN2 | |
| Renal ca. UO-31 | 0.0 | Pancreas Pool | 4.5 |

TABLE ASC
Panel 5 Islet

| | Rel. Exp. (%) | | Rel. Exp. (%) |
|---|---|---|---|
| | Ag5938, Run | | Ag5938, Run |
| Tissue Name | 248045753 | Tissue Name | 248045753 |
| 97457_Patient- | 0.0 | 94709_Donor 2 AM - A_adipose | 0.0 |
| 02go_adipose | | | |
| 97476_Patient- | 0.0 | 94710_Donor 2 AM - B_adipose | 0.0 |
| 07sk_skeletal muscle | | | |
| 97477_Patient- | 0.0 | 94711_Donor 2 AM - C_adipose | 0.0 |
| 07ut_uterus | | | |
| 97478_Patient- | 1.1 | 94712_Donor 2 AD - A_adipose | 0.0 |
| 07pl_placenta | | | |
| 99167_Bayer Patient 1 | 0.0 | 94713_Donor 2 AD - B_adipose | 0.0 |
| 97482_Patient- | 0.0 | 94714_Donor 2 AD - C_adipose | 0.0 |
| 08ut_uterus | | | |
| 97483_Patient- | 0.0 | 94742_Donor 3 U - | 0.0 |
| 08pl_placenta | | A_Mesenchymal Stem Cells | |
| 97486_Patient- | 0.0 | 94743_Donor 3 U - | 0.0 |
| 09sk_skeletal muscle | | B_Mesenchymal Stem Cells | |
| 97487_Patient- | 0.0 | 94730_Donor 3 AM - A_adipose | 0.7 |
| 09ut_uterus | | | |
| 97488_Patient- | 0.5 | 94731_Donor 3 AM - B_adipose | 0.0 |
| 09pl_placenta | | | |
| 97492_Patient- | 0.0 | 94732_Donor 3 AM - C_adipose | 0.0 |
| 10ut_uterus | | | |
| 97493_Patient- | 0.4 | 94733_Donor 3 AD - A_adipose | 0.0 |
| 10pl_placenta | | | |
| 97495_Patient- | 1.0 | 94734_Donor 3 AD - B_adipose | 0.0 |
| 11go_adipose | | | |
| 97496_Patient- | 2.4 | 94735_Donor 3 AD - C_adipose | 0.0 |
| 11sk_skeletal muscle | | | |
| 97497_Patient- | 0.0 | 77138_Liver_HepG2untreated | 0.0 |
| 11ut_uterus | | | |
| 97498_Patient- | 0.0 | 73556_Heart_Cardiac stromal | 0.0 |
| 11pl_placenta | | cells (primary) | |
| 97500_Patient- | 0.7 | 81735_Small Intestine | 100.0 |
| 12go_adipose | | | |
| 97501_Patient- | 6.8 | 72409_Kidney_Proximal | 0.0 |
| 12sk_skeletal muscle | | Convoluted Tubule | |
| 97502_Patient- | 0.0 | 82685_Small | 4.6 |
| 12ut_uterus | | intestine_Duodenum | |
| 97503_Patient- | 0.0 | 90650_Adrenal_Adrenocortical | 0.0 |
| 12pl_placenta | | adenoma | |
| 94721_Donor 2 U - | 0.0 | 72410_Kidney_HRCE | 0.0 |
| A_Mesenchymal | | | |
| Stem Cells | | | |
| 94722_Donor 2 U - | 0.0 | 72411_Kidney_HRE | 0.0 |
| B_Mesenchymal | | | |
| Stem Cells | | | |
| 94723_Donor 2 U - | 6.5 | 73139_Uterus_Uterine smooth | 0.0 |
| C_Mesenchymal | | muscle cells | |
| Stem Cells | | | |

General_screening_panel_v1.5 Summary: Ag5938 Highest expression of the CG59432-01 gene is seen in a gastric cancer cell line (CT=32.5). Thus, expression of this gene could be used to differentiate between this sample and other samples on this panel. In addition, low expression of this gene is seen in colon cancer CaCo-2, lung cancer NCI-H526, ovarian cancer OVCAR-5, and squamous cell carcinoma SCC-4 cell lines. Therefore, therapeutic modulation of the activity of this gene or its protein product, through the use of small molecule drugs, protein therapeutics or antibodies, might be beneficial in the treatment of these cancers.

Significant expression is also detected in fetal skeletal muscle and adult skeletal muscle (CT=32.5). At least 50 disease-causing mutations in the skeletal muscle voltage-gated chloride channel gene (CLCN1), almost all of which originate from Caucasian families, have been identified. Therefore, therapeutic modulation of this gene product, a chloride channel homolog, may be a treatment for myotonia congenita and other muscle channelopathies.

In addition, this gene is expressed at low levels in most regions of the central nervous system examined, including amygdala, substantia nigra, thalamus, and cerebral cortex. Therefore, this gene may play a role in central nervous system disorders such as Alzheimer's disease, Parkinson's disease, epilepsy, multiple sclerosis, schizophrenia and depression.

### REFERENCES

1. Sasaki R, Ito N, Shimamura M, Murakami T, Kuzuhara S, Uchino M, Uyama E. A novel CLCN1 mutation: P480T in a Japanese family with Thomsen's myotonia congenita. Muscle Nerve. March 2001;24(3):357-63.

Panel 5 Islet Summary: Ag5938 Expression of the CG59432-01 is restricted to a sample from small intestine (CT=31.6). Thus, expression of this gene could be used to differentiate between this sample and other samples on this panel and as a marker for this tissue.

AT. CG59383-01: D6MM5E

Expression of gene CG59383-01 was assessed using the primer-probe set Ag3427, described in Table ATA. Results of the RTQ-PCR runs are shown in Tables ATB, ATC and ATD.

Table AJTA. Probe Name Ag3427

TABLE ATA
Probe Name Ag3427

| | | | Start | SEQ ID |
|---|---|---|---|---|
| Primers | Sequences | Length | Position | NO: |
| Forward | 5′-cagtggaacagaccaagaaca-3′ | 21 | 784 | 532 |
| Probe | TET-5′-tctttcttcacagtgtttcagcaaca-3′-TAMRA | 26 | 817 | 533 |
| Reverse | 5′-ggattatctctgggtctggaa-3′ | 21 | 844 | 534 |

TABLE ATB
CNS_neurodegeneration_v1.0

| | Rel. Exp. (%) | | Rel. Exp. (%) |
|---|---|---|---|
| | Ag3427, Run | | Ag3427, Run |
| Tissue Name | 210351187 | Tissue Name | 210351187 |
| AD 1 Hippo | 11.4 | Control (Path) 3 | 2.0 |
| | | Temporal Ctx | |
| AD 2 Hippo | 50.3 | Control (Path) 4 | 23.3 |
| | | Temporal Ctx | |
| AD 3 Hippo | 10.2 | AD 1 Occipital Ctx | 9.8 |
| AD 4 Hippo | 17.6 | AD 2 Occipital Ctx | 0.0 |
| | | (Missing) | |
| AD 5 Hippo | 59.5 | AD 3 Occipital Ctx | 0.0 |
| AD 6 Hippo | 42.9 | AD 4 Occipital Ctx | 17.0 |
| Control 2 Hippo | 21.2 | AD 5 Occipital Ctx | 25.3 |
| Control 4 Hippo | 6.1 | AD 6 Occipital Ctx | 29.9 |
| Control (Path) 3 | 15.1 | Control 1 Occipital | 3.0 |
| Hippo | | Ctx | |
| AD 1 Temporal | 22.7 | Control 2 Occipital | 32.3 |
| Ctx | | Ctx | |
| AD 2 Temporal | 45.1 | Control 3 Occipital | 26.2 |
| Ctx | | Ctx | |
| AD 3 Temporal | 6.0 | Control 4 Occipital | 8.4 |
| Ctx | | Ctx | |
| AD 4 Temporal | 39.0 | Control (Path) 1 | 84.1 |
| Ctx | | Occipital Ctx | |
| AD 5 Inf | 100.0 | Control (Path) 2 | 11.7 |
| Temporal Ctx | | Occipital Ctx | |
| AD 5 Sup | 53.6 | Control (Path) 3 | 0.9 |
| Temporal Ctx | | Occipital Ctx | |
| AD 6 Inf | 57.0 | Control (Path) 4 | 10.3 |
| Temporal Ctx | | Occipital Ctx | |
| AD 6 Sup | 58.2 | Control 1 Parietal | 6.4 |
| Temporal Ctx | | Ctx | |
| Control 1 | 18.8 | Control 2 Parietal | 48.6 |
| Temporal Ctx | | Ctx | |
| Control 2 | 51.1 | Control 3 Parietal | 38.7 |
| Temporal Ctx | | Ctx | |
| Control 3 | 16.5 | Control (Path) 1 | 54.7 |
| Temporal Ctx | | Parietal Ctx | |
| Control 3 | 5.5 | Control (Path) 2 | 8.4 |
| Temporal Ctx | | Parietal Ctx | |
| Control (Path) 1 | 82.9 | Control (Path) 3 | 1.4 |
| Temporal Ctx | | Parietal Ctx | |
| Control (Path) 2 | 33.0 | Control (Path) 4 | 16.2 |
| Temporal Ctx | | Parietal Ctx | |

TABLE ATC
General_screening_panel_v1.4

| | Rel. Exp. (%) | | Rel. Exp. (%) |
|---|---|---|---|
| | Ag3427, Run | | Ag3427, Run |
| Tissue Name | 216821480 | Tissue Name | 216821480 |
| Adipose | 0.5 | Renal ca. TK-10 | 8.4 |
| Melanoma* Hs688(A).T | 0.0 | Bladder | 1.7 |
| Melanoma* | 0.1 | Gastric ca. (liver met.) | 0.0 |
| Hs688(B).T | | NCI-N87 | |
| Melanoma* M14 | 0.2 | Gastric ca. KATO III | 0.3 |
| Melanoma* | 0.0 | Colon ca. SW-948 | 0.0 |
| LOXIMVI | | | |
| Melanoma* SK- | 0.2 | Colon ca. SW480 | 0.4 |
| MEL-5 | | | |
| Squamous cell | 14.5 | Colon ca.* (SW480 | 0.1 |
| carcinoma SCC-4 | | met) SW620 | |
| Testis Pool | 10.7 | Colon ca. HT29 | 0.0 |
| Prostate ca.* (bone | 0.0 | Colon ca. HCT-116 | 100.0 |
| met) PC-3 | | | |
| Prostate Pool | 0.0 | Colon ca. CaCo-2 | 0.1 |
| Placenta | 0.2 | Colon cancer tissue | 1.0 |
| Uterus Pool | 0.0 | Colon ca. SW1116 | 0.0 |
| Ovarian ca. | 2.9 | Colon ca. Colo-205 | 0.0 |
| OVCAR-3 | | | |
| Ovarian ca. SK-OV-3 | 50.7 | Colon ca. SW-48 | 0.0 |
| Ovarian ca. | 2.1 | Colon Pool | 0.4 |
| OVCAR-4 | | | |
| Ovarian ca. | 1.1 | Small Intestine Pool | 0.3 |
| OVCAR-5 | | | |
| Ovarian ca. | 9.7 | Stomach Pool | 0.9 |
| IGROV-1 | | | |
| Ovarian ca. | 13.7 | Bone Marrow Pool | 0.4 |
| OVCAR-8 | | | |
| Ovary | 0.2 | Fetal Heart | 0.0 |
| Breast ca. MCF-7 | 0.1 | Heart Pool | 0.0 |
| Breast ca. MDA- | 0.0 | Lymph Node Pool | 0.4 |
| MB-231 | | | |
| Breast ca. BT 549 | 0.0 | Fetal Skeletal Muscle | 0.2 |
| Breast ca. T47D | 2.1 | Skeletal Muscle Pool | 0.0 |
| Breast ca. MDA-N | 0.0 | Spleen Pool | 0.2 |
| Breast Pool | 1.7 | Thymus Pool | 1.4 |
| Trachea | 1.7 | CNS cancer | 0.0 |
| | | (glio/astro) U87-MG | |
| Lung | 0.0 | CNS cancer | 0.4 |
| | | (glio/astro) U-118-MG | |
| Fetal Lung | 0.4 | CNS cancer | 0.1 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.3 | CNS cancer (astro) | 0.8 |
| | | SNB-75 | |
| Lung ca. NCI-H146 | 0.0 | CNS cancer (glio) | 13.9 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 1.7 |
| Lung ca. NCI-H526 | 0.0 | Brain (cerebellum) | 0.9 |
| Lung ca. NCI-H23 | 0.2 | Brain (fetal) | 0.1 |
| Lung ca. NCI-H460 | 0.0 | Brain (Hippocampus) | 2.2 |
| | | Pool | |
| Lung ca. HOP-62 | 0.5 | Cerebral Cortex Pool | 1.7 |
| Lung ca. NCI-H522 | 0.1 | Brain (Substantia nigra) Pool | 1.0 |
| Liver | 0.0 | Brain (Thalamus) Pool | 2.9 |
| Fetal Liver | 0.1 | Brain (whole) | 1.7 |
| Liver ca. HepG2 | 0.0 | Spinal Cord Pool | 0.2 |
| Kidney Pool | 0.2 | Adrenal Gland | 0.4 |
| Fetal Kidney | 0.8 | Pituitary gland Pool | 1.3 |
| Renal ca. 786-0 | 0.1 | Salivary Gland | 0.6 |
| Renal ca. A498 | 0.1 | Thyroid (female) | 1.9 |
| Renal ca. ACHN | 0.0 | Pancreatic ca. | 0.1 |
| | | CAPAN2 | |
| Renal ca. UO-31 | 0.5 | Pancreas Pool | 2.3 |

TABLE ATD
Panel 4D

| | Rel. Exp. (%) | | Rel. Exp. (%) |
|---|---|---|---|
| | Ag3427, Run | | Ag3427, Run |
| Tissue Name | 166396769 | Tissue Name | 166396769 |
| Secondary Th1 act | 1.4 | HUVEC IL-1beta | 0.0 |
| Secondary Th2 act | 0.0 | HUVEC IFN gamma | 0.0 |
| Secondary Tr1 act | 0.8 | HUVEC TNF alpha + | 0.0 |
| | | IFN gamma | |
| Secondary Th1 rest | 1.0 | HUVEC TNF alpha + | 0.0 |
| | | IL4 | |
| Secondary Th2 rest | 0.0 | HUVEC IL-11 | 0.0 |
| Secondary Tr1 rest | 2.9 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 4.9 | Microsvasular Dermal | 0.0 |
| | | EC TNF alpha + IL-1beta | |
| Primary Th1 rest | 2.5 | Bronchial epithelium | 7.9 |
| | | TNF alpha + IL-1beta | |
| Primary Th2 rest | 2.0 | Small airway epithelium | 0.0 |
| | | none | |
| Primary Tr1 rest | 0.6 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.9 | Coronery artery SMC | 0.0 |
| lymphocyte act | | TNF alpha + IL-1beta | |
| CD8 lymphocyte act | 0.0 | Astrocytes rest | 5.0 |
| Secondary CD8 | 1.0 | Astrocytes TNF alpha + | 2.4 |
| lymphocyte rest | | IL-1beta | |
| Secondary CD8 | 0.8 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 2.0 | CCD1106 | 28.5 |
| CD95 CH11 | | (Keratinocytes) none | |
| LAK cells rest | 0.0 | CCD1106 | 100.0 |
| | | (Keratinocytes) | |
| | | TNF alpha + IL-1beta | |
| LAK cells IL-2 | 0.0 | Liver cirrhosis | 23.8 |
| LAK cells IL-2 + IL-12 | 1.6 | Lupus kidney | 3.4 |
| LAK cells IL-2 + IFN | 3.7 | NCI-H292 none | 1.8 |
| gamma | | | |
| LAK cells IL-2 + IL-18 | 0.9 | NCI-H292 IL-4 | 3.8 |
| LAK cells | 0.0 | NCI-H292 IL-9 | 2.0 |
| PMA/ionomycin | | | |
| NK Cells IL-2 rest | 0.0 | NCI-H292 IL-13 | 4.0 |
| Two Way MLR 3 day | 0.0 | NCI-H292 IFN gamma | 1.1 |
| Two Way MLR 5 day | 0.0 | HPAEC none | 0.0 |
| Two Way MLR 7 day | 0.9 | HPAEC TNF alpha + IL- | 0.0 |
| | | 1beta | |
| PBMC rest | 0.0 | Lung fibroblast none | 0.0 |
| PBMC PWM | 0.7 | Lung fibroblast TNF | 0.8 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.7 |
| Ramos (B cell) | 0.0 | Lung fibroblast IL-13 | 0.0 |
| ionomycin | | | |
| B lymphocytes PWM | 3.4 | Lung fibroblast IFN | 0.0 |
| | | gamma | |
| B lymphocytes CD40L | 4.0 | Dermal fibroblast | 0.9 |
| and IL-4 | | CCD1070 rest | |
| EOL-1 dbcAMP | 0.0 | Dermal fibroblast | 5.6 |
| | | CCD1070 TNF alpha | |
| EOL-1 dbcAMP | 1.1 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 1.6 |
| Dendritic cells anti- | 0.0 | IBD Colitis 2 | 5.9 |
| CD40 | | | |
| Monocytes rest | 0.0 | IBD Crohn's | 2.4 |
| Monocytes LPS | 0.0 | Colon | 4.1 |
| Macrophages rest | 0.0 | Lung | 1.7 |
| Macrophages LPS | 0.0 | Thymus | 12.4 |
| HUVEC none | 0.0 | Kidney | 10.2 |
| HUVEC starved | 0.0 | | |

CNS_neurodegeneration_v1.0 Summary: Ag3427 This panel confirms the expression of CG59383-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3427 Highest expression of the CG59383-01 gene is seen in a colon cancer cell line (CT=27.2). Significant expression is also seen in a cluster of samples derived from ovarian cancer cell lines. Thus, expression of this gene could be used to differentiate between these samples and other samples on this panel and as a marker for the presence of these cancers. Furthermore, therapeutic modulation of the expression or function of this gene may be effective in the treatment of ovarian or colon cancers.

This molecule is also expressed at low levels in all regions of the CNS examined. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among tissues with metabolic function, this gene is expressed at low levels in adipose and pancreas. This expression suggests that this gene product may play a role in normal neuroendocrine and metabolic and that disregulated expression of this gene may contribute to neuroendocrine disorders or metabolic diseases, such as obesity and diabetes

Panel 4D Summary: Ag3427 Highest expression of the CG59383-01 gene is seen in keratinocytes treated with the inflammatory cytokines TNF-alpha and IL-1 beta (CT=30.3). Therefore, modulation of the expression or activity of the protein encoded by this transcript through the application of small molecule therapeutics may be useful in the treatment of asthma, COPD, emphysema, psoriasis and wound healing.

AU. CG58526-01: Scramblase

Expression of gene CG358526-01 was assessed using the primer-probe set Ag3366, described in Table AUA. Results of the RTQ-PCR runs are shown in Table AUB.

Table AUA. Probe Name Ag3366

TABLE AUA
Probe Name Ag3366

| | | | Start | SEQ ID |
|---|---|---|---|---|
| Primers | Sequences | Length | Position | NO: |
| Forward | 5′-tgtcttcacaaatgctgacaat-3′ | 22 | 729 | 535 |
| Probe | TET-5′-ttcggaattcatgttcctgcagatct-3′-TAMRA | 26 | 751 | 536 |
| Reverse | 5′-gatcattgctgctttgactgtt-3′ | 22 | 783 | 537 |

TABLE AUB
General_screening_panel_v1.4

| | Rel. Exp. (%) | | Rel. Exp. (%) |
|---|---|---|---|
| | Ag3366, Run | | Ag3366, Run |
| Tissue Name | 217042585 | Tissue Name | 217042585 |
| Adipose | 0.0 | Renal ca. TK-10 | 0.0 |
| Melanoma* Hs688(A).T | 0.0 | Bladder | 0.0 |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK-MEL-5 | 0.0 | Colon ca. SW480 | 0.0 |
| Squamous cell | 0.0 | Colon ca.* (SW480 | 100.0 |
| carcinoma SCC-4 | | met) SW620 | |
| Testis Pool | 69.3 | Colon ca. HT29 | 0.0 |
| Prostate ca.* (bone | 0.0 | Colon ca. HCT-116 | 25.2 |
| met) PC-3 | | | |
| Prostate Pool | 0.0 | Colon ca. CaCo-2 | 43.2 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 0.0 | Stomach Pool | 49.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 3.9 |
| Breast Pool | 0.0 | Thymus Pool | 0.0 |
| Trachea | 0.0 | CNS cancer | 3.5 |
| | | (glio/astro) U87-MG | |
| Lung | 0.0 | CNS cancer | 0.0 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) SF- | 0.0 |
| | | 539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) Pool | 12.5 |
| Lung ca. NCI-H526 | 0.0 | Brain (cerebellum) | 0.0 |
| Lung ca. NCI-H23 | 0.0 | Brain (fetal) | 13.8 |
| Lung ca. NCI-H460 | 0.0 | Brain (Hippocampus) Pool | 15.0 |
| Lung ca. HOP-62 | 0.0 | Cerebral Cortex Pool | 2.6 |
| Lung ca. NCI-H522 | 0.0 | Brain (Substantia | 7.5 |
| | | nigra) Pool | |
| Liver | 0.0 | Brain (Thalamus) Pool | 15.7 |
| Fetal Liver | 0.0 | Brain (whole) | 15.2 |
| Liver ca. HepG2 | 0.0 | Spinal Cord Pool | 7.5 |
| Kidney Pool | 0.0 | Adrenal Gland | 0.0 |
| Fetal Kidney | 0.0 | Pituitary gland Pool | 22.8 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

CNS_neurodegeneration_v1.0 Summary: Ag3366 Expression of the CG58526-01 gene is low/undetectable in all samples on this panel (CTs>35). (Data not shown.) General_screening_panel_v1.4 Summary: Ag3366 Expression of the CG58526-01 gene is restricted to a sample derived from a colon cancer cell line (CT=34.5) and the testis. Thus, expression of this gene could be used to differentiate between these samples and other samples on this panel and as a marker to detect the presence of colon cancer. Furthermore, therapeutic modulation of the expression or function of this gene may be effective in the treatment of colon cancer.

Panel 4D Summary: Ag3366 Results from one experiment with the CG58526-01 gene are not included. The amp plot indicates that there were experimental difficulties with this run.

AV. CG57851-01: Sulfotransferase

Expression of gene CG57851-01 was assessed using the primer-probe set Ag3349, described in Table AVA. Results of the RTQ-PCR runs are shown in Tables AVB, AVC and AVD.

Table AVA. Probe Name Ag3349

TABLE AVA
Probe Name Ag3349

| | | | Start | SEQ ID |
|---|---|---|---|---|
| Primers | Sequences | Length | Position | NO: |
| Forward | 5′-acaaaatgatggcgatattgag-3′ | 22 | 237 | 538 |
| Probe | TET-5′-cgcttccattcaacttcaacaccct-3′-TAMRA | 25 | 270 | 539 |
| Reverse | 5′-tcattcttatccactccaggaa-3′ | 22 | 295 | 540 |

TABLE AVB
CNS_neurodegeneration_v1.0

| | Rel. Exp. (%) | | Rel. Exp. (%) |
|---|---|---|---|
| | Ag3349, Run | | Ag3349, Run |
| Tissue Name | 210141483 | Tissue Name | 210141483 |
| AD 1 Hippo | 32.8 | Control (Path) 3 | 0.0 |
| | | Temporal Ctx | |
| AD 2 Hippo | 61.6 | Control (Path) 4 | 48.6 |
| | | Temporal Ctx | |
| AD 3 Hippo | 18.0 | AD 1 Occipital | 10.5 |
| | | Ctx | |
| AD 4 Hippo | 8.9 | AD 2 Occipital | 0.0 |
| | | Ctx (Missing) | |
| AD 5 hippo | 18.0 | AD 3 Occipital | 0.0 |
| | | Ctx | |
| AD 6 Hippo | 11.7 | AD 4 Occipital | 0.0 |
| | | Ctx | |
| Control 2 Hippo | 27.4 | AD 5 Occipital | 8.6 |
| | | Ctx | |
| Control 4 Hippo | 17.9 | AD 6 Occipital | 0.0 |
| | | Ctx | |
| Control (Path) 3 | 12.7 | Control 1 | 0.0 |
| Hippo | | Occipital Ctx | |
| AD 1 Temporal Ctx | 14.8 | Control 2 | 0.0 |
| | | Occipital Ctx | |
| AD 2 Temporal Ctx | 8.7 | Control 3 | 51.4 |
| | | Occipital Ctx | |
| AD 3 Temporal Ctx | 8.2 | Control 4 | 5.6 |
| | | Occipital Ctx | |
| AD 4 Temporal Ctx | 10.4 | Control (Path) 1 | 100.0 |
| | | Occipital Ctx | |
| AD 5 Inf Temporal | 7.2 | Control (Path) 2 | 17.8 |
| Ctx | | Occipital Ctx | |
| AD 5 Sup Temporal | 7.4 | Control (Path) 3 | 0.0 |
| Ctx | | Occipital Ctx | |
| AD 6 Inf Temporal | 9.1 | Control (Path) 4 | 41.2 |
| Ctx | | Occipital Ctx | |
| AD 6 Sup Temporal | 27.9 | Control 1 Parietal | 3.3 |
| Ctx | | Ctx | |
| Control 1 Temporal | 9.2 | Control 2 Parietal | 70.7 |
| Ctx | | Ctx | |
| Control 2 Temporal | 25.9 | Control 3 Parietal | 14.3 |
| Ctx | | Ctx | |
| Control 3 Temporal | 13.4 | Control (Path) 1 | 35.8 |
| Ctx | | Parietal Ctx | |
| Control 4 Temporal | 3.7 | Control (Path) 2 | 17.7 |
| Ctx | | Parietal Ctx | |
| Control (Path) 1 | 53.2 | Control (Path) 3 | 0.0 |
| Temporal Ctx | | Parietal Ctx | |
| Control (Path) 2 | 51.1 | Control (Path) 4 | 52.1 |
| Temporal Ctx | | Parietal Ctx | |

TABLE AVC
General_screening_panel_v1.4

| | Rel. Exp. (%) | | Rel. Exp. (%) |
|---|---|---|---|
| | Ag3349, Run | | Ag3349, Run |
| Tissue Name | 215620671 | Tissue Name | 215620671 |
| Adipose | 3.6 | Renal ca. TK-10 | 1.9 |
| Melanoma* Hs688(A).T | 4.5 | Bladder | 35.4 |
| Melanoma* | 0.3 | Gastric ca. (liver met.) | 0.4 |
| Hs688(B).T | | NCI-N87 | |
| Melanoma* M14 | 0.0 | Gastric ca. KATO III | 0.7 |
| Melanoma* | 0.5 | Colon ca. SW-948 | 2.8 |
| LOXIMVI | | | |
| Melanoma* SK-MEL-5 | 8.0 | Colon ca. SW480 | 10.2 |
| Squamous cell | 0.0 | Colon ca.* (SW480 | 13.5 |
| carcinoma SCC-4 | | met) SW620 | |
| Testis Pool | 5.4 | Colon ca. HT29 | 0.0 |
| Prostate ca.* (bone | 0.8 | Colon ca. HCT-116 | 0.7 |
| met) PC-3 | | | |
| Prostate Pool | 15.1 | Colon ca. CaCo-2 | 3.0 |
| Placenta | 0.0 | Colon cancer tissue | 7.0 |
| Uterus Pool | 0.4 | Colon ca. SW1116 | 0.2 |
| Ovarian ca. | 0.9 | Colon ca. Colo-205 | 0.8 |
| OVCAR-3 | | | |
| Ovarian ca. SK- | 18.4 | Colon ca. SW-48 | 0.0 |
| OV-3 | | | |
| Ovarian ca. | 0.0 | Colon Pool | 5.3 |
| OVCAR-4 | | | |
| Ovarian ca. | 10.4 | Small Intestine Pool | 2.7 |
| OVCAR-5 | | | |
| Ovarian ca. | 0.3 | Stomach Pool | 5.8 |
| IGROV-1 | | | |
| Ovarian ca. | 1.3 | Bone Marrow Pool | 1.7 |
| OVCAR-8 | | | |
| Ovary | 5.0 | Fetal Heart | 2.0 |
| Breast ca. MCF-7 | 1.0 | Heart Pool | 2.4 |
| Breast ca. MDA- | 1.3 | Lymph Node Pool | 8.9 |
| MB-231 | | | |
| Breast ca. BT 549 | 0.4 | Fetal Skeletal Muscle | 2.7 |
| Breast ca. T47D | 9.0 | Skeletal Muscle Pool | 0.0 |
| Breast ca. MDA-N | 1.6 | Spleen Pool | 0.4 |
| Breast Pool | 10.5 | Thymus Pool | 10.5 |
| Trachea | 1.2 | CNS cancer | 11.4 |
| | | (glio/astro) U87-MG | |
| Lung | 1.3 | CNS cancer | 2.4 |
| | | (glio/astro) U-118-MG | |
| Fetal Lung | 7.6 | CNS cancer | 0.1 |
| | | (neuro; met) SK-N-AS | |
| Lung ca. NCI-N417 | 0.3 | CNS cancer (astro) SF- | 0.2 |
| | | 539 | |
| Lung ca. LX-1 | 100.0 | CNS cancer (astro) | 4.6 |
| | | SNB-75 | |
| Lung ca. NCI-H146 | 0.8 | CNS cancer (glio) | 3.1 |
| | | SNB-19 | |
| Lung ca. SHP-77 | 0.0 | CNS cancer (glio) SF- | 5.3 |
| | | 295 | |
| Lung ca. A549 | 0.8 | Brain (Amygdala) Pool | 0.4 |
| Lung ca. NCI-H526 | 0.0 | Brain (cerebellum) | 0.9 |
| Lung ca. NCI-H23 | 7.1 | Brain (fetal) | 1.6 |
| Lung ca. NCI-H460 | 0.8 | Brain (Hippocampus) Pool | 0.8 |
| Lung ca. HOP-62 | 2.6 | Cerebral Cortex Pool | 2.3 |
| Lung ca. NCI-H522 | 0.4 | Brain (Substantia | 2.2 |
| | | nigra) Pool | |
| Liver | 0.0 | Brain (Thalamus) Pool | 2.8 |
| Fetal Liver | 10.5 | Brain (whole) | 3.1 |
| Liver ca. HepG2 | 0.8 | Spinal Cord Pool | 4.1 |
| Kidney Pool | 7.9 | Adrenal Gland | 2.4 |
| Fetal Kidney | 47.3 | Pituitary gland Pool | 1.4 |
| Renal ca. 786-0 | 2.6 | Salivary Gland | 1.4 |
| Renal ca. A498 | 0.9 | Thyroid (female) | 0.9 |
| Renal ca. ACHN | 1.3 | Pancreatic ca. | 3.7 |
| | | CAPAN2 | |
| Renal ca. UO-31 | 2.8 | Pancreas Pool | 9.3 |

TABLE AVD
Panel 4D

| | Rel. Exp. (%) | | Rel. Exp. (%) |
|---|---|---|---|
| | Ag3349, Run | | Ag3349, Run |
| Tissue Name | 165222879 | Tissue Name | 165222879 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.8 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 2.3 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 2.3 |
| | | EC none | |
| Primary Tr1 act | 0.0 | Microsvasular Dermal | 0.6 |
| | | EC TNF alpha + IL-1beta | |
| Primary Th1 rest | 0.0 | Bronchial epithelium | 0.0 |
| | | TNF alpha + IL-1beta | |
| Primary Th2 rest | 0.7 | Small airway epithelium | 0.4 |
| | | none | |
| Primary Tr1 rest | 0.0 | Small airway epithelium | 2.1 |
| | | TNF alpha + IL-1beta | |
| CD45RA CD4 | 0.0 | Coronery artery SMC rest | 0.6 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 1.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.6 |
| CD95 CH11 | | (Keratinocytes) none | |
| LAK cells rest | 1.4 | CCD1106 | 0.0 |
| | | (Keratinocytes) | |
| | | TNF alpha + IL-1beta | |
| LAK cells IL-2 | 0.0 | Liver cirrhosis | 3.3 |
| LAK cells IL-2 + IL-12 | 0.0 | Lupus kidney | 5.8 |
| LAK cells IL-2 + IFN | 1.0 | NCI-H292 none | 0.6 |
| gamma | | | |
| LAK cells IL-2 + IL-18 | 0.0 | NCI-H292 IL-4 | 0.0 |
| LAK cells | 1.3 | NCI-H292 IL-9 | 1.5 |
| PMA/ionomycin | | | |
| NK Cells IL-2 rest | 0.0 | NCI-H292 IL-13 | 0.8 |
| Two Way MLR 3 day | 0.0 | NCI-H292 IFN gamma | 0.3 |
| Two Way MLR 5 day | 0.0 | HPAEC none | 0.7 |
| Two Way MLR 7 day | 1.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.6 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.9 |
| Ramos (B cell) | 0.6 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.6 |
| and IL-4 | | CCD1070 rest | |
| EOL-1 dbcAMP | 0.0 | Dermal fibroblast | 0.8 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 7.4 | Dermal fibroblast IFN | 0.0 |
| | | gamma | |
| Dendritic cells LPS | 2.1 | Dermal fibroblast IL-4 | 0.7 |
| Dendritic cells anti- | 2.9 | IBD Colitis 2 | 0.0 |
| CD40 | | | |
| Monocytes rest | 0.0 | IBD Crohn's | 0.0 |
| Monocytes LPS | 1.3 | Colon | 0.6 |
| Macrophages rest | 0.9 | Lung | 0.7 |
| Macrophages LPS | 0.2 | Thymus | 100.0 |
| HUVEC none | 0.0 | Kidney | 1.7 |
| HUVEC starved | 0.0 | | |

CNS_neurodegeneration_v1.0 Summary: Ag3349 This panel confirms the expression of CG57851-01 gene at low levels in the brains of an independent group of individuals. However, no differential expression of this gene was detected between Alzheimer's diseased postmortem brains and those of non-demented controls in this experiment. The expression of this gene in the brain suggests that therapeutic modulation of the expression or function of this gene may be useful in the treatment of neurologic disorders, such as Alzheimer's disease, Parkinson's disease, schizophrenia, multiple sclerosis, stroke and epilepsy.

General_screening_panel_v1.4 Summary: Ag3349 Highest expression of the CG57851-01 gene is seen in a lung cancer cell line (CT=30). Thus, expression of this gene may be used to differentiate between this sample and other samples on this panel and as a marker for lung cancer. This gene encodes a sulfotransferase homolog. Sulfotransferases are involved in the metabolism of drugs and endogenous compounds in the body and also synthesize the complex glycoproteins found on the cell surface of cancer cells. Therefore, therapeutic modulation of the expression or function of this gene may be effective in the treatment of lung cancer.

Among tissues with metabolic function, this gene is expressed at moderate to low levels in adipose and pancreas. This expression among these tissues suggests that this gene product may play a role in normal metabolic function and that disregulated expression of this gene may contribute to metabolic diseases, such as obesity and diabetes.

Panel 4D Summary: Ag3349 Highest expression of the CG57851-01 gene is seen in the thymus (CT=29.7). The putative protein encoded by this gene could therefore play an important role in T cell development. Small molecule therapeutics designed against the protein encoded by this gene could be utilized to modulate immune function (T cell development) and be important for organ transplant, AIDS treatment or post chemotherapy immune reconstitution.

Panel 5 Islet Summary: Ag3349 Expression of the CG57851-01 gene is low/undetectable in all samples on this panel (CTs>35). (Data not shown.)

AW. CG59258-01: KIAA1608 Protein

Expression of gene CG59258-01 was assessed using the primer-probe set Ag3520, described in Table AWA.

Table AWA. Probe Name Ag3520

TABLE AWA
Probe Name Ag3520

| | | | Start | SEQ ID |
|---|---|---|---|---|
| Primers | Sequences | Length | Position | NO: |
| Forward | 5′-cctcacagatgaggacacaga-3′ | 21 | 717 | 541 |
| Probe | TET-5′acttgcttgccaaagtcactcagcaa-3′-TAMRA | 26 | 752 | 542 |
| Reverse | 5′-tttctgagagccagacagacat-3′ | 22 | 781 | 543 |

CNS_neurodegeneration_v1.0 Summary: Ag3520 Expression of the CG59258-01 gene is low/undetectable in all samples on this panel (CTs>35). (Data not shown.)

General_screening_panel_v1.4 Summary: Ag3520 Expression of the CG59258-01 gene is low/undetectable in all samples on this panel (CTs>35). (Data not shown.)

Panel 4D Summary: Ag3520 Expression of the CG59258-01 gene is low/undetectable in all samples on this panel (CTs>35). (Data not shown.)

AX. CG59564-01: Sorting Nexin 6

Expression of gene CG59564-01 was assessed using the primer-probe set Ag3471, described in Table AXA. Results of the RTQ-PCR runs are shown in Tables AXB, AXC and AXD.

Table AXA. Probe Name Ag3471

TABLE AXA
Probe Name Ag3471

| | | | Start | SEQ ID |
|---|---|---|---|---|
| Primers | Sequences | Length | Position | NO: |
| Forward | 5′-gtgcctggcagacgattata-3′ | 20 | 820 | 544 |
| Probe | TET-5′-ctatctcagctgcgctgagcagtctg-3′-TAMRA | 26 | 843 | 545 |
| Reverse | 5′-gtccttagctggttgacttcct-3′ | 22 | 876 | 546 |

TABLE AXB
CNS_neurodegeneration_v1.0

| | Rel. Exp. (%) | | Rel. Exp. (%) |
|---|---|---|---|
| | Ag3471, Run | | Ag3471, Run |
| Tissue Name | 210376963 | Tissue Name | 210376963 |
| AD 1 Hippo | 10.8 | Control (Path) 3 | 2.4 |
| | | Temporal Ctx | |
| AD 2 Hippo | 27.0 | Control (Path) 4 | 28.5 |
| | | Temporal Ctx | |
| AD 3 Hippo | 5.9 | AD 1 Occipital Ctx | 10.3 |
| AD 4 Hippo | 10.8 | AD 2 Occipital Ctx | 0.0 |
| | | (Missing) | |
| AD 5 Hippo | 64.6 | AD 3 Occipital Ctx | 3.6 |
| AD 6 Hippo | 43.8 | AD 4 Occipital Ctx | 30.4 |
| Control 2 Hippo | 56.3 | AD 5 Occipital Ctx | 100.0 |
| Control 4 Hippo | 4.0 | AD 6 Occipital Ctx | 14.5 |
| Control (Path) 3 | 2.3 | Control 1 Occipital | 1.1 |
| Hippo | | Ctx | |
| AD 1 Temporal | 11.5 | Control 2 Occipital | 82.9 |
| Ctx | | Ctx | |
| AD 2 Temporal | 36.3 | Control 3 Occipital | 13.3 |
| Ctx | | Ctx | |
| AD 3 Temporal | 4.7 | Control 4 Occipital | 5.4 |
| Ctx | | Ctx | |
| AD 4 Temporal | 27.9 | Control (Path) 1 | 87.7 |
| Ctx | | Occipital Ctx | |
| AD 5 Inf | 85.9 | Control (Path) 2 | 10.3 |
| Temporal Ctx | | Occipital Ctx | |
| AD 5 Sup | 37.1 | Control (Path) 3 | 1.5 |
| Temporal Ctx | | Occipital Ctx | |
| AD 6 Inf | 46.3 | Control (Path) 4 | 12.2 |
| Temporal Ctx | | Occipital Ctx | |
| AD 6 Sup | 52.1 | Control 1 Parietal | 3.1 |
| Temporal Ctx | | Ctx | |
| Control 1 | 3.6 | Control 2 Parietal | 38.4 |
| Temporal Ctx | | Ctx | |
| Control 2 | 81.2 | Control 3 Parietal | 17.4 |
| Temporal Ctx | | Ctx | |
| Control 3 | 19.1 | Control (Path) 1 | 88.9 |
| Temporal Ctx | | Parietal Ctx | |
| Control 3 | 8.0 | Control (Path) 2 | 22.2 |
| Temporal Ctx | | Parietal Ctx | |
| Control (Path) 1 | 88.9 | Control (Path) 3 | 1.7 |
| Temporal Ctx | | Parietal Ctx | |
| Control (Path) 2 | 48.0 | Control (Path) 4 | 38.7 |
| Temporal Ctx | | Parietal Ctx | |

TABLE AXC
General_screening_panel_v1.4

| | Rel. Exp. (%) | | Rel. Exp. (%) |
|---|---|---|---|
| | Ag3471, Run | | Ag3471, Run |
| Tissue Name | 222691297 | Tissue Name | 222691297 |
| Adipose | 2.3 | Renal ca. TK-10 | 2.7 |
| Melanoma* Hs688(A).T | 3.1 | Bladder | 4.2 |
| Melanoma* | 4.2 | Gastric ca. (liver met.) | 6.3 |
| Hs688(B).T | | NCI-N87 | |
| Melanoma* M14 | 13.0 | Gastric ca. KATO III | 4.1 |
| Melanoma* | 0.7 | Colon ca. SW-948 | 0.7 |
| LOXIMVI | | | |
| Melanoma* SK-MEL-5 | 0.8 | Colon ca. SW480 | 2.6 |
| Squamous cell | 1.5 | Colon ca.* (SW480 | 4.5 |
| carcinoma SCC-4 | | met) SW620 | |
| Testis Pool | 6.2 | Colon ca. HT29 | 2.2 |
| Prostate ca.* (bone | 3.8 | Colon ca. HCT-116 | 3.9 |
| met) PC-3 | | | |
| Prostate Pool | 0.7 | Colon ca. CaCo-2 | 3.5 |
| Placenta | 2.3 | Colon cancer tissue | 1.0 |
| Uterus Pool | 0.7 | Colon ca. SW1116 | 1.9 |
| Ovarian ca. | 5.3 | Colon ca. Colo-205 | 0.6 |
| OVCAR-3 | | | |
| Ovarian ca. SK- | 2.1 | Colon ca. SW-48 | 2.3 |
| OV-3 | | | |
| Ovarian ca. | 2.8 | Colon Pool | 7.0 |
| OVCAR-4 | | | |
| Ovarian ca. | 5.8 | Small Intestine Pool | 5.8 |
| OVCAR-5 | | | |
| Ovarian ca. | 5.3 | Stomach Pool | 5.5 |
| IGROV-1 | | | |
| Ovarian ca. | 3.0 | Bone Marrow Pool | 3.5 |
| OVCAR-8 | | | |
| Ovary | 7.1 | Fetal Heart | 1.7 |
| Breast ca. MCF-7 | 2.2 | Heart Pool | 3.1 |
| Breast ca. MDA- | 2.4 | Lymph Node Pool | 9.5 |
| MB-231 | | | |
| Breast ca. BT 549 | 98.6 | Fetal Skeletal Muscle | 1.4 |
| Breast ca. T47D | 8.4 | Skeletal Muscle Pool | 4.2 |
| Breast ca. MDA-N | 4.5 | Spleen Pool | 1.8 |
| Breast Pool | 7.5 | Thymus Pool | 6.2 |
| Trachea | 2.7 | CNS cancer | 7.0 |
| | | (glio/astro) U87-MG | |
| Lung | 1.9 | CNS cancer | 4.9 |
| | | (glio/astro) U-118-MG | |
| Fetal Lung | 13.4 | CNS cancer | 11.3 |
| | | (neuro; met) SK-N-AS | |
| Lung ca. NCI-N417 | 2.2 | CNS cancer (astro) SF- | 1.9 |
| | | 539 | |
| Lung ca. LX-1 | 2.2 | CNS cancer (astro) | 23.8 |
| | | SNB-75 | |
| Lung ca. NCI-H146 | 2.1 | CNS cancer (glio) | 3.6 |
| | | SNB-19 | |
| Lung ca. SHP-77 | 3.7 | CNS cancer (glio) SF- | 11.3 |
| | | 295 | |
| Lung ca. A549 | 3.7 | Brain (Amygdala) Pool | 22.1 |
| Lung ca. NCI-H526 | 2.5 | Brain (cerebellum) | 42.9 |
| Lung ca. NCI-H23 | 17.6 | Brain (fetal) | 100.0 |
| Lung ca. NCI-H460 | 1.8 | Brain (Hippocampus) Pool | 26.6 |
| Lung ca. HOP-62 | 3.6 | Cerebral Cortex Pool | 33.0 |
| Lung ca. NCI-H522 | 5.7 | Brain (Substantia | 29.3 |
| | | nigra) Pool | |
| Liver | 0.1 | Brain (Thalamus) Pool | 37.4 |
| Fetal Liver | 1.3 | Brain (whole) | 55.1 |
| Liver ca. HepG2 | 1.3 | Spinal Cord Pool | 7.7 |
| Kidney Pool | 10.6 | Adrenal Gland | 1.5 |
| Fetal Kidney | 6.7 | Pituitary gland Pool | 0.6 |
| Renal ca. 786-0 | 0.9 | Salivary Gland | 0.8 |
| Renal ca. A498 | 0.8 | Thyroid (female) | 0.8 |
| Renal ca. ACHN | 2.1 | Pancreatic ca. | 3.0 |
| | | CAPAN2 | |
| Renal ca. UO-31 | 3.4 | Pancreas Pool | 7.4 |

TABLE AXD
Panel 4D

| | Rel. Exp. (%) | | Rel. Exp. (%) |
|---|---|---|---|
| | Ag3471, Run | | Ag3471, Run |
| Tissue Name | 166417126 | Tissue Name | 166417126 |
| Secondary Th1 act | 10.2 | HUVEC IL-1beta | 2.3 |
| Secondary Th2 act | 11.2 | HUVEC IFN gamma | 7.8 |
| Secondary Tr1 act | 19.2 | HUVEC TNF alpha + | 4.7 |
| | | IFN gamma | |
| Secondary Th1 rest | 28.7 | HUVEC TNF alpha + | 6.3 |
| | | IL4 | |
| Secondary Th2 rest | 18.4 | HUVEC IL-11 | 6.2 |
| Secondary Tr1 rest | 24.5 | Lung Microvascular EC | 6.4 |
| | | none | |
| Primary Th1 act | 8.7 | Lung Microvascular EC | 7.1 |
| | | TNF alpha + IL-1beta | |
| Primary Th2 act | 20.4 | Microvascular Dermal | 6.9 |
| | | EC none | |
| Primary Tr1 act | 31.0 | Microsvasular Dermal | 2.5 |
| | | EC TNF alpha + IL-1beta | |
| Primary Th1 rest | 45.7 | Bronchial epithelium | 2.6 |
| | | TNF alpha + IL1beta | |
| Primary Th2 rest | 23.3 | Small airway epithelium | 3.1 |
| | | none | |
| Primary Tr1 rest | 25.3 | Small airway epithelium | 3.3 |
| | | TNF alpha + IL-1beta | |
| CD45RA CD4 | 9.7 | Coronery artery SMC rest | 3.9 |
| lymphocyte act | | | |
| CD45RO CD4 | 23.2 | Coronery artery SMC | 4.6 |
| lymphocyte act | | TNF alpha + IL-1beta | |
| CD8 lymphocyte act | 9.0 | Astrocytes rest | 12.7 |
| Secondary CD8 | 24.8 | Astrocytes TNF alpha + | 18.9 |
| lymphocyte rest | | IL-1beta | |
| Secondary CD8 | 19.9 | KU-812 (Basophil) rest | 26.2 |
| lymphocyte act | | | |
| CD4 lymphocyte none | 10.6 | KU-812 (Basophil) | 50.0 |
| | | PMA/ionomycin | |
| 2ry Th1/Th2/Tr1_anti- | 22.7 | CCD1106 | 6.9 |
| CD95 CH11 | | (Keratinocytes) none | |
| LAK cells rest | 6.4 | CCD1106 | 10.9 |
| | | (Keratinocytes) | |
| | | TNF alpha + IL-1beta | |
| LAK cells IL-2 | 27.5 | Liver cirrhosis | 15.3 |
| LAK cells IL-2 + IL-12 | 21.3 | Lupus Kidney | 4.2 |
| LAK cells IL-2 + IFN | 27.4 | NCI-H292 none | 5.8 |
| gamma | | | |
| LAK cells IL-2 + IL-18 | 22.7 | NCI-H292 IL-4 | 7.2 |
| LAK cells | 8.2 | NCI-H292 IL-9 | 3.6 |
| PMA/ionomycin | | | |
| NK Cells IL-2 rest | 13.6 | NCI-H292 IL-13 | 3.7 |
| Two Way MLR 3 day | 23.7 | NCI-H292 IFN gamma | 3.2 |
| Two Way MLR 5 day | 5.6 | HPAEC none | 3.7 |
| Two Way MLR 7 day | 7.7 | HPAEC TNF alpha + IL- | 2.3 |
| | | 1beta | |
| PBMC rest | 6.1 | Lung fibroblast none | 18.3 |
| PBMC PWM | 7.3 | Lung fibroblast TNF | 20.6 |
| | | alpha + IL-1beta | |
| PBMC PHA-L | 7.1 | Lung fibroblast IL-4 | 16.6 |
| Ramos (B cell) none | 6.3 | Lung fibroblast IL-9 | 9.2 |
| Ramos (B cell) | 3.2 | Lung fibroblast IL-13 | 7.9 |
| ionomycin | | | |
| B lymphocytes PWM | 12.2 | Lung fibroblast IFN | 6.2 |
| | | gamma | |
| B lymphocytes CD40L | 11.1 | Dermal fibroblast | 11.4 |
| and IL-4 | | CCD1070 rest | |
| EOL-1 dbcAMP | 7.4 | Dermal fibroblast | 28.5 |
| | | CCD1070 TNF alpha | |
| EOL-1 dbcAMP | 12.5 | Dermal fibroblast | 7.2 |
| PMA/ionomycin | | CCD1070 IL-1beta | |
| Dendritic cells none | 13.4 | Dermal fibroblast IFN | 6.9 |
| | | gamma | |
| Dendritic cells LPS | 14.0 | Dermal Fibroblasts IL-4 | 12.8 |
| Dendritic cells anti- | 15.9 | IBD Colitis 2 | 1.7 |
| CD40 | | | |
| Monocytes rest | 21.3 | IBD Crohn's | 4.8 |
| Monocytes LPS | 11.4 | Colon | 100.0 |
| Macrophages rest | 23.5 | Lung | 12.6 |
| Macrophages LPS | 3.7 | Thymus | 8.9 |
| HUVEC none | 6.7 | Kidney | 34.4 |
| HUVEC starved | 11.0 | | |

CNS_neurodegeneration_v1.0 Summary: Ag3471 This panel does not show differential expression of the CG59564-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3471 The CG59564-01 gene, a sorting nexin homolog, shows highly brain preferential expression. Moderate levels of expression are seen in all brain regions examined, with highest expression in the fetal brain (CT=28.5). Thus, this gene would be useful for distinguishing brain tissue from non-neural tissue, and may be beneficial as a drug target in neurologic disease, such as Alzheimer's disease, Parkinson's disease, schizophrenia, multiple sclerosis, stroke and epilepsy.

Among tissues with metabolic function, this gene is expressed at low levels in pituitary, adipose, adrenal gland, pancreas, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at significant levels in a breast cancer cell line (CT=28.6). Thus, expression of this gene could be used to differentiate this sample from other samples on this panel and as a marker for breast cancer.

Panel 4D Summary: Ag3471 The CG59564-01 gene, a sorting nexin homolog, is most highly expressed in normal colon (CT=30). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AY. CG59553-01: Secretory Protein SEC8

Expression of gene CG59553-01 was assessed using the primer-probe set Ag3465, described in Table AYA. Results of the RTQ-PCR runs are shown in Tables AYB, AYC and AYD.

Table AYA. Probe Name Ag3465

TABLE AYA
Probe Name Ag3465

| | | | Start | SEQ ID |
|---|---|---|---|---|
| Primers | Sequences | Length | Position | NO: |
| Forward | 5′-ttcacagcaagaagatgaacct-3′ | 22 | 616 | 547 |
| Probe | TET-5′-tcatagatgaactacaccggcacctg-3′-TAMRA | 26 | 649 | 548 |
| Reverse | 5′-ctcggctagtcgatttgatgt-3′ | 21 | 676 | 549 |

TABLE AYB
CNS_neurodegeneration_v1.0

| | Rel. Exp. (%) | | Rel. Exp. (%) |
|---|---|---|---|
| | Ag3465, Run | | Ag3465, Run |
| Tissue Name | 210376516 | Tissue Name | 210376516 |
| AD 1 Hippo | 21.3 | Control (Path) 3 | 8.2 |
| | | Temporal Ctx | |
| AD 2 Hippo | 33.0 | Control (Path) 4 | 40.9 |
| | | Temporal Ctx | |
| AD 3 Hippo | 11.0 | AD 1 Occipital Ctx | 20.7 |
| AD 4 Hippo | 11.6 | AD 2 Occipital Ctx | 0.0 |
| | | (Missing) | |
| AD 5 Hippo | 87.7 | AD 3 Occipital Ctx | 10.3 |
| AD 6 Hippo | 46.7 | AD 4 Occipital Ctx | 24.8 |
| Control 2 Hippo | 29.7 | AD 5 Occipital Ctx | 40.6 |
| Control 4 Hippo | 20.4 | AD 6 Occipital Ctx | 25.3 |
| Control (Path) 3 | 14.2 | Control 1 Occipital | 6.7 |
| Hippo | | Ctx | |
| AD 1 Temporal | 21.3 | Control 2 Occipital | 59.9 |
| Ctx | | Ctx | |
| AD 2 Temporal | 38.7 | Control 3 Occipital | 21.8 |
| Ctx | | Ctx | |
| AD 3 Temporal | 8.2 | Control 4 Occipital | 11.8 |
| Ctx | | Ctx | |
| AD 4 Temporal | 30.8 | Control (Path) 1 | 79.6 |
| Ctx | | Occipital Ctx | |
| AD 5 Inf | 100.0 | Control (Path) 2 | 18.8 |
| Temporal Ctx | | Occipital Ctx | |
| AD 5 Sup | 58.2 | Control (Path) 3 | 4.0 |
| Temporal Ctx | | Occipital Ctx | |
| AD 6 Inf | 47.6 | Control (Path) 4 | 25.7 |
| Temporal Ctx | | Occipital Ctx | |
| AD 6 Sup | 52.1 | Control 1 Parietal | 14.2 |
| Temporal Ctx | | Ctx | |
| Control 1 | 11.8 | Control 2 Parietal | 56.6 |
| Temporal Ctx | | Ctx | |
| Control 2 | 42.0 | Control 3 Parietal | 23.8 |
| Temporal Ctx | | Ctx | |
| Control 3 | 22.8 | Control (Path) 1 | 75.3 |
| Temporal Ctx | | Parietal Ctx | |
| Control 3 | 14.0 | Control (Path) 2 | 29.7 |
| Temporal Ctx | | Parietal Ctx | |
| Control (Path) 1 | 64.6 | Control (Path) 3 | 8.5 |
| Temporal Ctx | | Parietal Ctx | |
| Control (Path) 2 | 47.0 | Control (Path) 4 | 52.9 |
| Temporal Ctx | | Parietal Ctx | |

TABLE AYC
General_screening_panel_v1.4

| | Rel. Exp. (%) | | Rel. Exp. (%) |
|---|---|---|---|
| | Ag3465, Run | | Ag3465, Run |
| Tissue Name | 217118990 | Tissue Name | 217118990 |
| Adipose | 13.1 | Renal ca. TK-10 | 60.7 |
| Melanoma* Hs688(A).T | 22.5 | Bladder | 37.9 |
| Melanoma* | 30.1 | Gastric ca. (liver met.) | 42.9 |
| Hs688(B).T | | NCI-N87 | |
| Melanoma* M14 | 54.7 | Gastric ca. KATO III | 48.0 |
| Melanoma* | 15.5 | Colon ca. SW-948 | 5.5 |
| LOXIMVI | | | |
| Melanoma* SK-MEL-5 | 42.0 | Colon ca. SW480 | 57.4 |
| Squamous cell | 8.7 | Colon ca.* (SW480 | 37.4 |
| carcinoma SCC-4 | | met) SW620 | |
| Testis Pool | 12.8 | Colon ca. HT29 | 25.0 |
| Prostate ca.* (bone | 63.7 | Colon ca. HCT-116 | 28.3 |
| met) PC-3 | | | |
| Prostate Pool | 13.0 | Colon ca. CaCo-2 | 46.7 |
| Placenta | 7.1 | Colon cancer tissue | 26.4 |
| Uterus Pool | 12.0 | Colon ca. SW1116 | 8.6 |
| Ovarian ca. | 37.4 | Colon ca. Colo-205 | 6.9 |
| OVCAR-3 | | | |
| Ovarian ca. SK- | 21.5 | Colon ca. SW-48 | 7.5 |
| OV-3 | | | |
| Ovarian ca. | 26.1 | Colon Pool | 21.8 |
| OVCAR-4 | | | |
| Ovarian ca. | 42.0 | Small Intestine Pool | 25.5 |
| OVCAR-5 | | | |
| Ovarian ca. | 23.5 | Stomach Pool | 15.4 |
| IGROV-1 | | | |
| Ovarian ca. | 24.7 | Bone Marrow Pool | 9.4 |
| OVCAR-8 | | | |
| Ovary | 14.0 | Fetal Heart | 6.9 |
| Breast ca. MCF-7 | 38.4 | Heart Pool | 11.1 |
| Breast ca. MDA- | 49.0 | Lymph Node Pool | 23.8 |
| MB-231 | | | |
| Breast ca. BT 549 | 45.4 | Fetal Skeletal Muscle | 11.9 |
| Breast ca. T47D | 74.7 | Skeletal Muscle Pool | 26.2 |
| Breast ca. MDA-N | 20.4 | Spleen Pool | 39.2 |
| Breast Pool | 22.8 | Thymus Pool | 39.8 |
| Trachea | 15.4 | CNS cancer | 100.0 |
| | | (glio/astro) U87-MG | |
| Lung | 6.8 | CNS cancer | 54.7 |
| | | (glio/astro) U-118-MG | |
| Fetal Lung | 41.5 | CNS cancer | 50.0 |
| | | (neuro; met) SK-N-AS | |
| Lung ca. NCI-N417 | 12.2 | CNS cancer (astro) SF- | 19.3 |
| | | 539 | |
| Lung ca. LX-1 | 26.1 | CNS cancer (astro) | 75.3 |
| | | SNB-75 | |
| Lung ca. NCI-H146 | 12.6 | CNS cancer (glio) | 23.8 |
| | | SNB-19 | |
| Lung ca. SHP-77 | 33.9 | CNS cancer (glio) SF- | 95.3 |
| | | 295 | |
| Lung ca. A549 | 43.8 | Brain (Amygdala) Pool | 11.6 |
| Lung ca. NCI-H526 | 7.6 | Brain (cerebellum) | 12.2 |
| Lung ca. NCI-H23 | 78.5 | Brain (fetal) | 32.5 |
| Lung ca. NCI-H460 | 25.0 | Brain (Hippocampus) Pool | 12.7 |
| Lung ca. HOP-62 | 28.5 | Cerebral Cortex Pool | 15.8 |
| Lung ca. NCI-H522 | 25.0 | Brain (Substantia | 11.7 |
| | | nigra) Pool | |
| Liver | 2.0 | Brain (Thalamus) Pool | 17.7 |
| Fetal Liver | 18.7 | Brain (whole) | 15.6 |
| Liver ca. HepG2 | 15.5 | Spinal Cord Pool | 12.5 |
| Kidney Pool | 36.6 | Adrenal Gland | 17.7 |
| Fetal Kidney | 26.8 | Pituitary gland Pool | 6.0 |
| Renal ca. 786-0 | 55.5 | Salivary Gland | 7.0 |
| Renal ca. A498 | 19.8 | Thyroid (female) | 6.6 |
| Renal ca. ACHN | 31.0 | Pancreatic ca. | 40.3 |
| | | CAPAN2 | |
| Renal ca. UO-31 | 48.0 | Pancreas Pool | 28.7 |

TABLE AYD
Panel 4D

| | Rel. Exp. (%) | | Rel. Exp. (%) |
|---|---|---|---|
| | Ag3465, Run | | Ag3465, Run |
| Tissue Name | 166417102 | Tissue Name | 166417102 |
| Secondary Th1 act | 22.1 | HUVEC IL-1beta | 14.6 |
| Secondary Th2 act | 33.9 | HUVEC IFN gamma | 17.8 |
| Secondary Tr1 act | 44.4 | HUVEC TNF alpha + | 10.6 |
| | | IFN gamma | |
| Secondary Th1 rest | 33.4 | HUVEC TNF alpha + | 8.3 |
| | | IL4 | |
| Secondary Th2 rest | 25.0 | HUVEC IL-11 | 8.2 |
| Secondary Tr1 rest | 29.7 | Lung Microvascular EC | 12.3 |
| | | none | |
| Primary Th1 act | 14.3 | Lung Microvascular EC | 14.8 |
| | | TNF alpha + IL-1beta | |
| Primary Th2 act | 41.2 | Microvascular Dermal | 15.5 |
| | | EC none | |
| Primary Tr1 act | 46.7 | Microsvasular Dermal | 14.7 |
| | | EC TNF alpha + IL-1beta | |
| Primary Th1 rest | 88.9 | Bronchial epithelium | 15.5 |
| | | TNF alpha + IL1beta | |
| Primary Th2 rest | 39.2 | Small airway epithelium | 14.0 |
| | | none | |
| Primary Tr1 rest | 31.0 | Small airway epithelium | 65.5 |
| | | TNF alpha + IL-1beta | |
| CD45RA CD4 | 20.6 | Coronery artery SMC rest | 18.3 |
| lymphocyte act | | | |
| CD45RO CD4 | 29.9 | Coronery artery SMC | 12.5 |
| lymphocyte act | | TNF alpha + IL-1beta | |
| CD8 lymphocyte act | 23.0 | Astrocytes rest | 28.7 |
| Secondary CD8 | 24.1 | Astrocytes TNF alpha + | 31.6 |
| lymphocyte rest | | IL-1beta | |
| Secondary CD8 | 18.7 | KU-812 (Basophil) rest | 19.8 |
| lymphocyte act | | | |
| CD4 lymphocyte none | 19.5 | KU-812 (Basophil) | 42.6 |
| | | PMA/ionomycin | |
| 2ry Th1/Th2/Tr1_anti- | 37.4 | CCD1106 | 21.8 |
| CD95 CH11 | | (Keratinocytes) none | |
| LAK cells rest | 17.1 | CCD1106 | 100.0 |
| | | (Keratinocytes) | |
| | | TNF alpha + IL-1beta | |
| LAK cells IL-2 | 35.8 | Liver cirrhosis | 16.5 |
| LAK cells IL-2 + IL-12 | 32.3 | Lupus kidney | 23.5 |
| LAK cells IL-2 + IFN | 38.4 | NCI-H292 none | 48.6 |
| gamma | | | |
| LAK cells IL-2 + IL-18 | 32.5 | NCI-H292 IL-4 | 45.1 |
| LAK cells | 12.0 | NCI-H292 IL-9 | 49.7 |
| PMA/ionomycin | | | |
| NK Cells IL-2 rest | 24.7 | NCI-H292 IL-13 | 26.4 |
| Two Way MLR 3 day | 31.4 | NCI-H292 IFN gamma | 25.3 |
| Two Way MLR 5 day | 19.6 | HPAEC none | 17.9 |
| Two Way MLR 7 day | 14.9 | HPAEC TNF alpha + IL- | 20.2 |
| | | 1beta | |
| PBMC rest | 18.4 | Lung fibroblast none | 39.2 |
| PBMC PWM | 18.7 | Lung fibroblast TNF | 32.8 |
| | | alpha + IL-1beta | |
| PBMC PHA-L | 10.2 | Lung fibroblast IL-4 | 28.3 |
| Ramos (B cell) none | 61.6 | Lung fibroblast IL-9 | 20.4 |
| Ramos (B cell) | 46.7 | Lung fibroblast IL-13 | 19.5 |
| ionomycin | | | |
| B lymphocytes PWM | 28.1 | Lung fibroblast IFN | 26.6 |
| | | gamma | |
| B lymphocytes CD40L | 44.8 | Dermal fibroblast | 26.8 |
| and IL-4 | | CCD1070 rest | |
| EOL-1 dbcAMP | 33.2 | Dermal fibroblast | 50.7 |
| | | CCD1070 TNF alpha | |
| EOL-1 dbcAMP | 25.5 | Dermal fibroblast | 18.4 |
| PMA/ionomycin | | CCD1070 IL-1beta | |
| Dendritic cells none | 30.1 | Dermal fibroblast IFN | 19.2 |
| | | gamma | |
| Dendritic cells LPS | 19.1 | Dermal fibroblast IL-4 | 34.6 |
| Dendritic cells anti- | 33.7 | IBD Colitis 2 | 9.0 |
| CD40 | | | |
| Monocytes rest | 25.0 | IBD Crohn's | 12.4 |
| Monocytes LPS | 16.3 | Colon | 56.3 |
| Macrophages rest | 44.4 | Lung | 16.4 |
| Macrophages LPS | 14.4 | Thymys | 49.3 |
| HUVEC none | 20.4 | Kidney | 52.1 |
| HUVEC starved | 37.1 | | |

CNS_neurodegeneration_v1.0 Summary: Ag3465 This panel does not show differential expression of the CG59553-01 gene in Alzheimer's disease. However, this expression profile confirms the presence of this gene in the brain. Please see Panel 1.4 for discussion of utility of this gene in the central nervous system.

General_screening_panel_v1.4 Summary: Ag3465 Highest expression of the CG59553-01 gene is seen in a brain cancer cell line (CTs=24). Expression of this gene is ubiquitous throughout this panel, with significant levels of expression in clusters of cell lines derived from brain, renal, colon, lung, breast, ovarian, and melanoma cancers. These high levels of expression in all the samples on this panel suggest a role for this gene in cell growth and proliferation.

This molecule is also expressed at high levels in all regions of the CNS examined. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

Panel 4D Summary: Ag3465 The CG59553-01 gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AZ. CG59435-01 and CG59435-02: Human Nedd1

Expression of gene CG59435-01 and CG59435-02 was assessed using the primer-probe set Ag3437, described in Table AZA. Results of the RTQ-PCR runs are shown in Tables AZB, AZC and AZD. Please note that CG59435-02 represents a full-length physical clone of the CG59435-01 gene, validating the prediction of the gene sequence.

Table AZA. Probe Name Ag3437

TABLE AZA
Probe Name Ag3437

| | | | Start | SEQ ID |
|---|---|---|---|---|
| Primers | Sequences | Length | Position | NO: |
| Forward | 5′-tggtgctgaaagtggaaatc-3′ | 20 | 1536 | 550 |
| Probe | TET-5′-cctctccatcatctaaccaaacaaga-3′-TAMRA | 26 | 1562 | 1551 |
| Reverse | 5′-tgggcttcaatttcattctct-3′ | 21 | 1611 | 552 |

TABLE AZB
CNS_neurodegeneration_v1.0

| | Rel. | | Rel. |
|---|---|---|---|
| | Exp. (%) | | Exp. (%) |
| | Ag3437, | | Ag3437, |
| | Run | | Run |
| Tissue Name | 210374394 | Tissue Name | 210374394 |
| AD 1 Hippo | 8.9 | Control (Path) 3 | 6.9 |
| | | Temporal Ctx | |
| AD 2 Hippo | 25.7 | Control (Path) 4 | 27.9 |
| | | Temporal Ctx | |
| AD 3 Hippo | 18.2 | AD 1 Occipital | 26.6 |
| | | Ctx | |
| AD 4 Hippo | 13.2 | AD 2 Occipital | 0.0 |
| | | Ctx (Missing) | |
| AD 5 hippo | 52.9 | AD 3 Occipital | 7.6 |
| | | Ctx | |
| AD 6 Hippo | 100.0 | AD 4 Occipital | 26.6 |
| | | Ctx | |
| Control 2 Hippo | 26.1 | AD 5 Occipital | 26.8 |
| | | Ctx | |
| Control 4 Hippo | 26.2 | AD 6 Occipital | 21.8 |
| | | Ctx | |
| Control (Path) 3 | 14.7 | Control 1 | 9.0 |
| Hippo | | Occipital Ctx | |
| AD 1 Temporal Ctx | 35.8 | Control 2 | 23.5 |
| | | Occipital Ctx | |
| AD 2 Temporal Ctx | 27.7 | Control 3 | 17.8 |
| | | Occipital Ctx | |
| AD 3 Temporal Ctx | 14.6 | Control 4 | 14.6 |
| | | Occipital Ctx | |
| AD 4 Temporal Ctx | 23.3 | Control (Path) 1 | 70.2 |
| | | Occipital Ctx | |
| AD 5 Inf Temporal | 65.5 | Control (Path) 2 | 12.9 |
| Ctx | | Occipital Ctx | |
| AD 5 SupTemporal | 47.0 | Control (Path) 3 | 4.9 |
| Ctx | | Occipital Ctx | |
| AD 6 Inf Temporal | 78.5 | Control (Path) 4 | 22.2 |
| Ctx | | Occipital Ctx | |
| AD 6 Sup Temporal | 92.0 | Control 1 Parietal | 13.3 |
| Ctx | | Ctx | |
| Control 1 Temporal | 10.9 | Control 2 Parietal | 50.0 |
| Ctx | | Ctx | |
| Control 2 Temporal | 23.5 | Control 3 Parietal | 13.1 |
| Ctx | | Ctx | |
| Control 3 Temporal | 17.9 | Control (Path) 1 | 35.4 |
| Ctx | | Parietal Ctx | |
| Control 4 Temporal | 12.8 | Control (Path) 2 | 26.6 |
| Ctx | | Parietal Ctx | |
| Control (Path) 1 | 37.4 | Control (Path) 3 | 5.4 |
| Temporal Ctx | | Parietal Ctx | |
| Control (Path) 2 | 44.4 | Control (Path) 4 | 29.3 |
| Temporal Ctx | | Parietal Ctx | |

TABLE AZC
General_screening_panel_v1.4

| | Rel. | | Rel. |
|---|---|---|---|
| | Exp. (%) | | Exp. (%) |
| | Ag3437, | | Ag3437, |
| | Run | | Run |
| Tissue Name | 217066730 | Tissue Name | 217066730 |
| Adipose | 10.0 | Renal ca. TK-10 | 24.0 |
| Melanoma* | 25.7 | Bladder | 18.6 |
| Hs688(A).T | | | |
| Melanoma* | 27.5 | Gastric ca. (liver met.) | 100.0 |
| Hs688(B).T | | NCI-N87 | |
| Melanoma* M14 | 34.9 | Gastric ca. KATO III | 60.3 |
| Melanoma* | 31.9 | Colon ca. SW-948 | 9.7 |
| LOXIMVI | | | |
| Melanoma* SK- | 8.7 | Colon ca. SW480 | 61.6 |
| MEL-5 | | | |
| Squamous cell | 24.8 | Colon ca.* (SW480 | 46.3 |
| carcinoma SCC-4 | | met) SW620 | |
| Testis Pool | 25.9 | Colon ca. HT29 | 22.7 |
| Prostate ca.* (bone | 84.1 | Colon ca. HCT-116 | 72.2 |
| met) PC-3 | | | |
| Prostate Pool | 12.3 | Colon ca. CaCo-2 | 32.8 |
| Placenta | 0.3 | Colon cancer tissue | 38.2 |
| Uterus Pool | 11.4 | Colon ca. SW1116 | 7.5 |
| Ovarian ca. | 33.2 | Colon ca. Colo-205 | 6.7 |
| OVCAR-3 | | | |
| Ovarian ca. SK- | 92.7 | Colon ca. SW-48 | 7.1 |
| OV-3 | | | |
| Ovarian ca. | 9.7 | Colon Pool | 27.2 |
| OVCAR-4 | | | |
| Ovarian ca. | 26.4 | Small Intestine Pool | 22.8 |
| OVCAR-5 | | | |
| Ovarian ca. | 16.5 | Stomach Pool | 12.3 |
| IGROV-1 | | | |
| Ovarian ca. | 6.5 | Bone Marrow Pool | 13.5 |
| OVCAR-8 | | | |
| Ovary | 7.6 | Fetal Heart | 21.9 |
| Breast ca. MCF-7 | 24.3 | Heart Pool | 11.7 |
| Breast ca. MDA- | 84.1 | Lymph Node Pool | 30.4 |
| MB-231 | | | |
| Breast ca. BT 549 | 68.3 | Fetal Skeletal Muscle | 15.2 |
| Breast ca. T47D | 52.1 | Skeletal Muscle Pool | 28.5 |
| Breast ca. MDA-N | 18.9 | Spleen Pool | 15.8 |
| Breast Pool | 26.6 | Thymus Pool | 21.2 |
| Trachea | 9.2 | CNS cancer | 14.0 |
| | | (glio/astro) U87-MG | |
| Lung | 4.9 | CNS cancer | 91.4 |
| | | (glio/astro) U-118-MG | |
| Fetal Lung | 49.0 | CNS cancer | 55.5 |
| | | (neuro; met) SK-N-AS | |
| Lung ca. NCI-N417 | 4.9 | CNS cancer (astro) | 14.5 |
| | | SF-539 | |
| Lung ca. LX-1 | 37.4 | CNS cancer (astro) | 33.0 |
| | | SNB-75 | |
| Lung ca. NCI-H146 | 6.8 | CNS cancer (glio) | 12.5 |
| | | SNB-19 | |
| Lung ca. SHP-77 | 51.4 | CNS cancer (glio) | 51.4 |
| | | SF-295 | |
| Lung ca. A549 | 33.9 | Brain (Amygdala) | 0.0 |
| | | Pool | |
| Lung ca. NCI-H526 | 8.4 | Brain (cerebellum) | 1.5 |
| Lung ca. NCI-H23 | 34.4 | Brain (fetal) | 6.2 |
| Lung ca. NCI-H460 | 98.6 | Brain (Hippocampus) | 3.0 |
| | | Pool | |
| Lung ca. HOP-62 | 15.2 | Cerebral Cortex Pool | 3.0 |
| Lung ca. NCI-H522 | 37.9 | Brain (Substantia | 3.6 |
| | | nigra) Pool | |
| Liver | 0.4 | Brain (Thalamus) Pool | 3.9 |
| Fetal Liver | 26.8 | Brain (whole) | 1.1 |
| Liver ca. HepG2 | 11.3 | Spinal Cord Pool | 5.0 |
| Kidney Pool | 23.2 | Adrenal Gland | 3.3 |
| Fetal Kidney | 42.9 | Pituitary gland Pool | 4.5 |
| Renal ca. 786-0 | 41.5 | Salivary Gland | 0.9 |
| Renal ca. A498 | 12.9 | Thyroid (female) | 4.2 |
| Renal ca. ACHN | 20.2 | Pancreatic ca. | 20.7 |
| | | CAPAN2 | |
| Renal ca. UO-31 | 29.7 | Pancreas Pool | 19.8 |

TABLE AZD
Panel 4.1D

| | Rel. Exp. (%) | | Rel. Exp. (%) |
|---|---|---|---|
| | Ag3437, Run | | Ag3437, Run |
| Tissue Name | 169839068 | Tissue Name | 169839068 |
| Secondary Th1 act | 44.8 | HUVEC IL-1beta | 23.2 |
| Secondary Th2 act | 57.8 | HUVEC IFN gamma | 26.2 |
| Secondary Tr1 act | 60.7 | HUVEC TNF alpha + | 18.6 |
| | | IFN gamma | |
| Secondary Th1 rest | 10.2 | HUVEC TNF alpha + | 16.7 |
| | | IL4 | |
| Secondary Th2 rest | 14.3 | HUVEC IL-11 | 10.9 |
| Secondary Tr1 rest | 13.9 | Lung Microvascular EC | 28.1 |
| | | none | |
| Primary Th1 act | 37.4 | Lung Microvascular EC | 25.3 |
| | | TNF alpha + IL-1beta | |
| Primary Th2 act | 34.9 | Microvascular Dermal | 19.9 |
| | | EC none | |
| Primary Tr1 act | 39.0 | Microsvasular Dermal | 17.3 |
| | | EC TNF alpha + IL-1beta | |
| Primary Th1 rest | 17.8 | Bronchial epithelium | 20.9 |
| | | TNF alpha + IL1beta | |
| Primary Th2 rest | 14.7 | Small airway epithelium | 4.9 |
| | | none | |
| Primary Tr1 rest | 23.2 | Small airway epithelium | 20.4 |
| | | TNF alpha + IL-1beta | |
| CD45RA CD4 | 39.0 | Coronery artery SMC rest | 10.7 |
| lymphocyte act | | | |
| CD45RO CD4 | 37.4 | Coronery artery SMC | 10.7 |
| lymphocyte act | | TNF alpha + IL-1beta | |
| CD8 lymphocyte act | 31.9 | Astrocytes rest | 9.2 |
| Secondary CD8 | 33.7 | Astrocytes TNF alpha + | 6.4 |
| lymphocyte rest | | IL-1beta | |
| Secondary CD8 | 21.8 | KU-812 (Basophil) rest | 36.6 |
| lymphocyte act | | | |
| CD4 lymphocyte none | 10.4 | KU-812 (Basophil) | 100.0 |
| | | PMA/ionomycin | |
| 2ry Th1/Th2/Tr1_anti- | 15.7 | CCD1106 | 21.9 |
| CD95 CH11 | | (Keratinocytes) none | |
| LAK cells rest | 21.8 | CCD1106 | 29.9 |
| | | (Keratinocytes) | |
| | | TNF alpha + IL-1beta | |
| LAK cells IL-2 | 24.1 | Liver cirrhosis | 6.0 |
| LAK cells IL-2 + IL-12 | 33.2 | NCI-H292 none | 13.0 |
| LAK cells IL-2 + IFN | 38.4 | NCI-H292 IL-4 | 25.2 |
| gamma | | | |
| LAK cells IL-2 + IL-18 | 33.9 | NCI-H292 IL-9 | 32.8 |
| LAK cells | 9.3 | NCI-H292 IL-13 | 26.2 |
| PMA/ionomycin | | | |
| NK Cells IL-2 rest | 27.9 | NCI-H292 IFN gamma | 37.9 |
| Two Way MLR 3 day | 23.2 | HPAEC none | 13.7 |
| Two Way MLR 5 day | 25.3 | HPAEC TNF alpha + IL- | 30.8 |
| | | 1beta | |
| Two Way MLR 7 day | 23.8 | Lung fibroblast none | 12.1 |
| PBMC rest | 9.1 | Lung fibroblast TNF | 9.5 |
| | | alpha + IL-1beta | |
| PBMC PWM | 25.9 | Lung fibroblast IL-4 | 11.7 |
| PBMC PHA-L | 27.7 | Lung fibroblast IL-9 | 19.3 |
| Ramos (B cell) none | 23.5 | Lung fibroblast IL-13 | 11.2 |
| Ramos (B cell) | 23.0 | Lung fibroblast IFN | 19.5 |
| ionomycin | | gamma | |
| B lymphocytes PWM | 36.3 | Dermal fibroblast | 66.9 |
| | | CCD1070 rest | |
| B lymphocytes CD40L | 21.5 | Dermal fibroblast | 70.2 |
| and IL-4 | | CCD1070 TNF alpha | |
| EOL-1 dbcAMP | 21.0 | Dermal fibroblast | 46.3 |
| | | CCD1070 IL-1beta | |
| EOL-1 dbcAMP | 19.8 | Dermal fibroblast IFN | 17.1 |
| PMA/ionomycin | | gamma | |
| Dendritic cells none | 10.2 | Dermal fibroblast IL-4 | 21.5 |
| Dendritic cells LPS | 10.8 | Dermal Fibroblasts rest | 8.9 |
| Dendritic cells anti- | 9.1 | Neutrophils TNFa + LPS | 0.5 |
| CD40 | | | |
| Monocytes rest | 10.1 | Neutrophils rest | 5.7 |
| Monocytes LPS | 11.6 | Colon | 5.5 |
| Macrophages rest | 13.9 | Lung | 10.7 |
| Macrophages LPS | 15.7 | Thymus | 39.2 |
| HUVEC none | 11.8 | Kidney | 8.8 |
| HUVEC starved | 18.7 | | |

CNS_neurodegeneration_v1.0 Summary: Ag3437 This panel confirms the expression of CG59435-01 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3437 The CG59435-01 is gene is ubiquitously expressed in this panel, with highest expression in a gastric cancer cell line (CT=26.5). In addition, significant levels of expression are evident in cell lines from brain cancer, colon cancer, ovarian cancer, breast cancer, prostate cancer and lung cancer. Thus, expression of this gene could be used as a marker to detect the presence of these cancers. Furthermore, therapeutic modulation of the expression or function of this gene may be effective in the treatment of these cancers.

In addition, this gene is expressed at moderate to low levels in pituitary, adipose, adrenal gland, pancreas, thyroid, and adult and fetal skeletal muscle, heart, and liver. This widespread expression among metabolic tissues suggests that this gene product may play a role in normal neuroendocrine and metabolic and that disregulated expression of this gene may contribute to neuroendocrine disorders or metabolic diseases, such as obesity and diabetes.

In addition, the CG59435-01 gene encodes a homologue of mouse NEDD1 protein. Nedd is an acronym of “neural precursor cell expressed developmentally and down-regulated” (Ref 1) The developmentally regulated mouse gene Nedd1 encodes a protein with similarities to the beta subunit of heterotrimeric GTP-binding proteins that has growth suppressing activity when overexpressed in various cultured cell types. Neddl mRNA is shown to be strongly expressed in early embryonic brain and may play a role in the differentiation-coupled growth arrest in neuronal cells (Ref. 2). The moderate to low levels (CT=30-0.33) in all regions of the central nervous system examined suggest that this gene product may also play a role in the differentiation-coupled growth arrest in neuronal cells. Furthermore, this gene may play a role in central nervous system disorders such as Alzheimer's disease, Parkinson's disease, epilepsy, multiple sclerosis, schizophrenia and depression.

### REFERENCES

1. Kumar S, Tomooka Y, Noda M. (1992) Identification of a set of genes with developmentally down-regulated expression in the mouse brain. Biochem Biophys Res Commun 185(3):1155-61

2. Kumar S, Matsuzaki T, Yoshida Y, Noda M. (1994) Molecular cloning and biological activity of a novel developmentally regulated gene encoding a protein with beta-transducin-like structure. J Biol Chem 269(15):11318-26.

Panel 4.1D Summary: Ag3437 The CG59435-01 is gene is ubiquitously expressed in this panel, with highest expression in the basophil cell line KU-812 treated with PMA/ionomycin (CT=27.9). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

BA. CG59439-01 and CG59439-02: Xenobiotic/Medium-Chain Fatty acid:CoA Ligase Form XL-III

Expression of gene CG59439-01 was assessed using the primer-probe set Ag3438, described in Table BAA. Results of the RTQ-PCR runs are shown in Table BAB. Please note that CG59439-02 represents a full-length physical clone of the CG59439-01 gene, validating the prediction of the gene sequence.

Table BAA. Probe Name Ag3438

TABLE BAA
Probe Name Ag3438

| | | | Start | SEQ ID |
|---|---|---|---|---|
| Primers | Sequences | Length | Position | NO: |
| Forward | 5′-accccattaaccacttttgg-3′ | 20 | 938 | 553 |
| Probe | TET-5′-tcatctatatatcgaatgattctgcagca-3′-TAMRA | 29 | 964 | 554 |
| Reverse | 5′-gaacctgatgctggtgaaatc-3′ | 21 | 994 | 555 |

TABLE BAB
Panel 4.1D

| | Rel. Exp. (%) | | Rel. Exp. (%) |
|---|---|---|---|
| | Ag3438, Run | | Ag3438, Run |
| Tissue Name | 198383568 | Tissue Name | 198383568 |
| Secondary Th1 act | 4.0 | HUVEC IL-1beta | 0.0 |
| Secondary Th2 act | 100.0 | HUVEC IFN gamma | 0.0 |
| Secondary Tr1 act | 25.9 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 9.4 |
| | | TNF alpha + IL1beta | |
| Primary Th2 rest | 0.0 | Small airway epithelium | 7.0 |
| | | none | |
| Primary Tr1 rest | 0.0 | Small airway epithelium | 13.8 |
| | | TNF alpha + IL-1beta | |
| CD45RA CD4 | 16.3 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 25.9 |
| lymphocyte act | | | |
| CD4 lymphocyte none | 12.9 | KU-812 (Basophil) | 10.5 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 6.9 | CCD1106 | 0.0 |
| | | (Keratinocytes) | |
| | | TNF alpha + IL-1beta | |
| LAK cells IL-2 | 6.1 | Liver cirrhosis | 20.4 |
| LAK cells IL-2 + IL-12 | 7.7 | NCI-H292 none | 0.0 |
| LAK cells IL-2 + IFN | 13.6 | NCI-H292 IL-4 | 6.7 |
| gamma | | | |
| LAK cells IL-2 + IL-18 | 14.2 | NCI-H292 IL-9 | 11.7 |
| LAK cells | 0.0 | NCI-H292 IL-13 | 25.9 |
| PMA/ionomycin | | | |
| NK Cells IL-2 rest | 0.0 | NCI-H292 IFN gamma | 17.2 |
| Two Way MLR 3 day | 14.9 | HPAEC none | 0.0 |
| Two Way MLR 5 day | 0.0 | HPAEC TNF alpha + IL- | 0.0 |
| | | 1beta | |
| Two Way MLR 7 day | 0.0 | Lung fibroblast none | 0.0 |
| PBMC rest | 4.4 | Lung fibroblast TNF | 0.0 |
| | | alpha + IL-1beta | |
| PBMC PWM | 2.7 | Lung fibroblast IL-4 | 0.0 |
| PBMC PHA-L | 21.2 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 11.1 | Dermal fibroblast | 0.0 |
| and IL-4 | | CCD1070 TNF alpha | |
| EOL-1 dbcAMP | 12.2 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 6.9 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 6.4 | Neutrophils rest | 4.8 |
| Monocytes LPS | 0.0 | Colon | 5.5 |
| Macrophages rest | 7.0 | Lung | 0.0 |
| Macrophages LPS | 0.0 | Thymus | 3.6 |
| HUVEC none | 0.0 | Kidney | 22.4 |
| HUVEC starved | 0.0 | | |

CNS_neurodegeneration_v1.0 Summary: Ag3438 Expression of the CG59439-02 gene is low/undetectable in all samples on this panel (CTs>35). (Data not shown.)

General_screening_panel_v1.4 Summary: Ag3438 Results from one experiment with the CG5;9439-02 gene are not included. The amp plot indicates that there were experimental difficulties with this run.

Panel 4.1D Summary: Ag3438 Expression of the CG59439-02 gene is restricted to a sample derived from chronically activated Th2 cells (CT=33).

Panel 41D Summary: Ag3438 Results from one experiment with the CG59439-02 gene are not included. The amp plot indicates that there were experimental difficulties with this run.

BB. CG59354-01 and CG59354-02 and CG59354-03: Phosducin-Like Protein

Expression of gene CG59354-01 and variant CG59354-02 was assessed using the primer-probe set Ag3553, described in Table BBA. Results of the RTQ-PCR runs are shown in Tables BBB, BBC and BBD. Please note that CG59354-03 represents a full-length physical clone of the CG59354-01 gene, validating the prediction of the gene sequence.

TABLE BBA
Probe Name Ag3553

| | | | Start | SEQ ID |
|---|---|---|---|---|
| Primers | Sequences | Length | Position | NO: |
| Forward | 5′-tctatttccaggtcgctatcct-3′ | 22 | 1778 | 556 |
| Probe | TET-5′-acgcacagatgtcagcaccaagactt-3′-TAMRA | 26 | 1822 | 557 |
| Reverse | 5′-ggaatttggattactcccagaa-3′ | 22 | 1852 | 558 |

TABLE BBB
CNS_neurodegeneration_v1.0

| | Rel. | | Rel. |
|---|---|---|---|
| | Exp. (%) | | Exp. (%) |
| | Ag3553, | | Ag3553, |
| | Run | | Run |
| Tissue Name | 210641082 | Tissue Name | 210641082 |
| AD 1 Hippo | 11.3 | Control (Path) 3 | 3.7 |
| | | Temporal Ctx | |
| AD 2 Hippo | 17.8 | Control (Path) 4 | 19.6 |
| | | Temporal Ctx | |
| AD 3 Hippo | 4.8 | AD 1 Occipital | 15.6 |
| | | Ctx | |
| AD 4 Hippo | 4.6 | AD 2 Occipital | 0.0 |
| | | Ctx (Missing) | |
| AD 5 hippo | 70.2 | AD 3 Occipital | 4.5 |
| | | Ctx | |
| AD 6 Hippo | 55.5 | AD 4 Occipital | 12.5 |
| | | Ctx | |
| Control 2 Hippo | 20.3 | AD 5 Occipital | 28.7 |
| | | Ctx | |
| Control 4 Hippo | 14.5 | AD 6 Occipital | 32.1 |
| | | Ctx | |
| Control (Path) 3 | 7.0 | Control 1 Occipital | 3.5 |
| Hippo | | Ctx | |
| AD 1 Temporal Ctx | 15.1 | Control 2 Occipital | 51.1 |
| | | Ctx | |
| AD 2 Temporal Ctx | 18.8 | Control 3 Occipital | 14.4 |
| | | Ctx | |
| AD 3 Temporal Ctx | 3.4 | Control 4 Occipital | 4.7 |
| | | Ctx | |
| AD 4 Temporal Ctx | 11.7 | Control (Path) 1 | 64.6 |
| | | Occipital Ctx | |
| AD 5 Inf Temporal | 100.0 | Control (Path) 2 | 8.0 |
| Ctx | | Occipital Ctx | |
| AD 5 SupTemporal | 50.7 | Control (Path) 3 | 3.9 |
| Ctx | | Occipital Ctx | |
| AD 6 Inf Temporal | 69.7 | Control (Path) 4 | 11.8 |
| Ctx | | Occipital Ctx | |
| AD 6 Sup Temporal | 66.9 | Control 1 Parietal | 7.0 |
| Ctx | | Ctx | |
| Control 1 Temporal | 4.8 | Control 2 Parietal | 41.2 |
| Ctx | | Ctx | |
| Control 2 Temporal | 26.6 | Control 3 Parietal | 12.0 |
| Ctx | | Ctx | |
| Control 3 Temporal | 9.1 | Control (Path) 1 | 62.0 |
| Ctx | | Parietal Ctx | |
| Control 4 Temporal | 7.5 | Control (Path) 2 | 21.6 |
| Ctx | | Parietal Ctx | |
| Control (Path) 1 | 44.8 | Control (Path) 3 | 3.5 |
| Temporal Ctx | | Parietal Ctx | |
| Control (Path) 2 | 24.8 | Control (Path) 4 | 41.8 |
| Temporal Ctx | | Parietal Ctx | |

TABLE BBC
General_screening_panel_v1.4

| | Rel. | | Rel. |
|---|---|---|---|
| | Exp. (%) | | Exp. (%) |
| | Ag3553, | | Ag3553, |
| | Run | | Run |
| Tissue Name | 217049381 | Tissue Name | 217049381 |
| Adipose | 5.2 | Renal ca. TK-10 | 40.6 |
| Melanoma* | 40.3 | Bladder | 29.9 |
| Hs688(A).T | | | |
| Melanoma* | 31.9 | Gastric ca. (liver met.) | 39.5 |
| Hs688(B).T | | NCI-N87 | |
| Melanoma* M14 | 41.5 | Gastric ca. KATO III | 55.9 |
| Melanoma* | 37.1 | Colon ca. SW-948 | 9.0 |
| LOXIMVI | | | |
| Melanoma* SK- | 25.9 | Colon ca. SW480 | 68.3 |
| MEL-5 | | | |
| Squamous cell | 17.9 | Colon ca.* (SW480 | 23.5 |
| carcinoma SCC-4 | | met) SW620 | |
| Testis Pool | 5.4 | Colon ca. HT29 | 20.0 |
| Prostate ca.* (bone | 31.4 | Colon ca. HCT-116 | 66.0 |
| met) PC-3 | | | |
| Prostate Pool | 9.0 | Colon ca. CaCo-2 | 32.8 |
| Placenta | 4.0 | Colon cancer tissue | 17.4 |
| Uterus Pool | 5.5 | Colon ca. SW1116 | 4.0 |
| Ovarian ca. | 40.1 | Colon ca. Colo-205 | 11.3 |
| OVCAR-3 | | | |
| Ovarian ca. SK- | 47.3 | Colon ca. SW-48 | 9.9 |
| OV-3 | | | |
| Ovarian ca. | 11.4 | Colon Pool | 18.4 |
| OVCAR-4 | | | |
| Ovarian ca. | 37.9 | Small Intestine Pool | 11.7 |
| OVCAR-5 | | | |
| Ovarian ca. | 25.0 | Stomach Pool | 14.9 |
| IGROV-1 | | | |
| Ovarian ca. | 16.6 | Bone Marrow Pool | 6.9 |
| OVCAR-8 | | | |
| Ovary | 10.2 | Fetal Heart | 4.6 |
| Breast ca. MCF-7 | 42.6 | Heart Pool | 6.6 |
| Breast ca. MDA- | 50.7 | Lymph Node Pool | 21.3 |
| MB-231 | | | |
| Breast ca. BT 549 | 81.8 | Fetal Skeletal Muscle | 3.8 |
| Breast ca. T47D | 85.9 | Skeletal Muscle Pool | 6.0 |
| Breast ca. MDA-N | 33.0 | Spleen Pool | 13.9 |
| Breast Pool | 17.3 | Thymus Pool | 11.1 |
| Trachea | 16.4 | CNS cancer | 44.4 |
| | | (glio/astro) U87-MG | |
| Lung | 5.3 | CNS cancer | 45.4 |
| | | (glio/astro) U-118-MG | |
| Fetal Lung | 33.7 | CNS cancer | 44.8 |
| | | (neuro; met) SK-N-AS | |
| Lung ca. NCI-N417 | 5.8 | CNS cancer (astro) | 31.0 |
| | | SF-539 | |
| Lung ca. LX-1 | 22.7 | CNS cancer (astro) | 100.0 |
| | | SNB-75 | |
| Lung ca. NCI-H146 | 16.7 | CNS cancer (glio) | 27.4 |
| | | SNB-19 | |
| Lung ca. SHP-77 | 59.5 | CNS cancer (glio) | 59.5 |
| | | SF-295 | |
| Lung ca. A549 | 41.5 | Brain (Amygdala) | 8.3 |
| | | Pool | |
| Lung ca. NCI-H526 | 5.4 | Brain (cerebellum) | 8.0 |
| Lung ca. NCI-H23 | 31.0 | Brain (fetal) | 23.8 |
| Lung ca. NCI-H460 | 33.4 | Brain (Hippocampus) | 10.7 |
| | | Pool | |
| Lung ca. HOP-62 | 21.9 | Cerebral Cortex Pool | 15.1 |
| Lung ca. NCI-H522 | 17.8 | Brain (Substantia | 11.0 |
| | | nigra) Pool | |
| Liver | 0.5 | Brain (Thalamus) Pool | 16.6 |
| Fetal Liver | 18.3 | Brain (whole) | 11.8 |
| Liver ca. HepG2 | 9.2 | Spinal Cord Pool | 11.7 |
| Kidney Pool | 29.5 | Adrenal Gland | 6.5 |
| Fetal Kidney | 16.2 | Pituitary gland Pool | 4.7 |
| Renal ca. 786-0 | 57.0 | Salivary Gland | 9.2 |
| Renal ca. A498 | 7.9 | Thyroid (female) | 8.3 |
| Renal ca. ACHN | 20.9 | Pancreatic ca. | 30.4 |
| | | CAPAN2 | |
| Renal ca. UO-31 | 29.1 | Pancreas Pool | 21.3 |

TABLE BBD
Panel 4D

| | Rel. Exp. (%) | | Rel. Exp. (%) |
|---|---|---|---|
| | Ag3553, Run | | Ag3553, Run |
| Tissue Name | 166487505 | Tissue Name | 166487505 |
| Secondary Th1 act | 45.4 | HUVEC IL-1beta | 17.0 |
| Secondary Th2 act | 42.9 | HUVEC IFN gamma | 39.2 |
| Secondary Tr1 act | 58.6 | HUVEC TNF alpha + | 27.4 |
| | | IFN gamma | |
| Secondary Th1 rest | 6.7 | HUVEC TNF alpha + | 31.4 |
| | | IL4 | |
| Secondary Th2 rest | 14.5 | HUVEC IL-11 | 23.7 |
| Secondary Tr1 rest | 15.0 | Lung Microvascular EC | 41.2 |
| | | none | |
| Primary Th1 act | 38.4 | Lung Microvascular EC | 28.3 |
| | | TNF alpha + IL-1beta | |
| Primary Th2 act | 24.5 | Microvascular Dermal | 59.0 |
| | | EC none | |
| Primary Tr1 act | 32.8 | Microsvasular Dermal | 23.5 |
| | | EC TNF alpha + IL-1beta | |
| Primary Th1 rest | 57.0 | Bronchial epithelium | 21.5 |
| | | TNF alpha + IL1beta | |
| Primary Th2 rest | 43.2 | Small airway epithelium | 5.8 |
| | | none | |
| Primary Tr1 rest | 39.0 | Small airway epithelium | 52.5 |
| | | TNF alpha + IL-1beta | |
| CD45RA CD4 | 25.5 | Coronery artery SMC rest | 14.3 |
| lymphocyte act | | | |
| CD45RO CD4 | 34.6 | Coronery artery SMC | 12.0 |
| lymphocyte act | | TNF alpha + IL-1beta | |
| CD8 lymphocyte act | 28.1 | Astrocytes rest | 18.2 |
| Secondary CD8 | 30.8 | Astrocytes TNF alpha + | 11.3 |
| lymphocyte rest | | IL-1beta | |
| Secondary CD8 | 21.9 | KU-812 (Basophil) rest | 19.6 |
| lymphocyte act | | | |
| CD4 lymphocyte none | 6.0 | KU-812 (Basophil) | 71.2 |
| | | PMA/ionomycin | |
| 2ry Th1/Th2/Tr1_anti- | 21.9 | CCD1106 | 31.6 |
| CD95 CH11 | | (Keratinocytes) none | |
| LAK cells rest | 24.8 | CCD1106 | 9.0 |
| | | (Keratinocytes) | |
| | | TNF alpha + IL-1beta | |
| LAK cells IL-2 | 32.8 | Liver cirrhosis | 4.1 |
| LAK cells IL-2 + IL-12 | 24.0 | Lupus kidney | 4.0 |
| LAK cells IL-2 + IFN | 37.6 | NCI-H292 none | 35.1 |
| gamma | | | |
| LAK cells IL-2 + IL-18 | 27.2 | NCI-H292 IL-4 | 39.2 |
| LAK cells | 16.8 | NCI-H292 IL-9 | 49.0 |
| PMA/ionomycin | | | |
| NK Cells IL-2 rest | 23.5 | NCI-H292 IL-13 | 28.7 |
| Two Way MLR 3 day | 21.5 | NCI-H292 IFN gamma | 29.5 |
| Two Way MLR 5 day | 16.3 | HPAEC none | 38.2 |
| Two Way MLR 7 day | 17.6 | HPAEC TNF alpha + IL- | 27.2 |
| | | 1beta | |
| PBMC rest | 8.0 | Lung fibroblast none | 9.4 |
| PBMC PWM | 91.4 | Lung fibroblast TNF | 11.1 |
| | | alpha + IL-1beta | |
| PBMC PHA-L | 33.9 | Lung fibroblast IL-4 | 34.6 |
| Ramos (B cell) none | 29.5 | Lung fibroblast IL-9 | 21.3 |
| Ramos (B cell) | 85.3 | Lung fibroblast IL-13 | 15.8 |
| ionomycin | | | |
| B lymphocytes PWM | 100.0 | Lung fibroblast IFN | 39.2 |
| | | gamma | |
| B lymphocytes CD40L | 33.0 | Dermal fibroblast | 45.1 |
| and IL-4 | | CCD1070 rest | |
| EOL-1 dbcAMP | 35.8 | Dermal fibroblast | 75.3 |
| | | CCD1070 TNF alpha | |
| EOL-1 dbcAMP | 46.7 | Dermal fibroblast | 37.4 |
| PMA/ionomycin | | CCD1070 IL-1beta | |
| Dendritic cells none | 14.3 | Dermal fibroblast IFN | 15.7 |
| | | gamma | |
| Dendritic cells LPS | 15.2 | Dermal fibroblast IL-4 | 25.9 |
| Dendritic cells anti- | 22.2 | IBD Colitis 2 | 1.4 |
| CD40 | | | |
| Monocytes rest | 10.4 | IBD Crohn's | 1.5 |
| Monocytes LPS | 16.7 | Colon | 16.7 |
| Macrophages rest | 27.9 | Lung | 12.2 |
| Macrophages LPS | 5.7 | Thymus | 28.7 |
| HUVEC none | 29.5 | Kidney | 28.5 |
| HUVEC starved | 67.4 | | |

CNS_neurodegeneration_v1.0 Summary: Ag3553 This panel confirms the expression of CG59354-03 gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3553 The CG59354-03 gene is ubiquitously expressed in this panel, with highest expression in a brain cancer cell line (CT=25.9). In addition, significant levels of expression are seen in cell lines derived from colon, breast, ovarian, renal, lung, prostate, and melanoma cancers. Furthermore, higher levels of expression are seen in fetal liver and lung (CTs=27-28) when compared to expression in the adult tissues (CTs=30-33). The high levels of expression in fetal tissue and cancer cell lines, both of which are highly proliferative, suggests that this gene product may be involved in cell growth and differentiation. Therefore, therapeutic modulation of the expression or function of this gene may be effective in the treatment of cancer.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases;, such as obesity and diabetes.

In addition, this gene is expressed at high levels in all regions of the central nervous system examined, including amygdala, hippocampus, substantia nigra, thalamus, cerebellum, cerebral cortex, and spinal cord. The CG59354-03 gene encodes a splice variant of phosphoducin-like protein (PHLP). PDCL is a putative modulator of heterotrimeric G proteins. It was initially isolated as the product of an ethanol-responsive gene in neural cell cultures (Ref. 1). PDCL shares extensive amino acid sequence homology with phosducin (PDC), a phosphoprotein expressed in retina and pineal gland that inhibits several G protein-coupled signaling pathways by binding to the beta-gamma subunits, of G proteins. Therefore, this gene may play a role in central nervous system disorders such as Alzheimer's disease, Parkinson's disease, epilepsy, multiple sclerosis, schizophrenia and depression.

### REFERENCES

1. Miles M F, Barhite S, Sganga M, Elliott M. (1993) Phosducin-like protein: an ethanol.-responsive potential modulator of guanine nucleotide-binding protein function. Proc Natl Acad Sci USA 90(22):10831-5

Panel 4D Summary: Ag3553 The CG59354-03 gene is ubiquitously expressed in this panel, with highest expression in B cells treated with polk-weed mitogen (CT=27.2). In addition, this gene is expressd at is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

BC. CG59319-01 and CG59319-02: Phosducin-Like Protein

Expression of gene CG59319-01 was assessed using the primer-probe set Ag3544, described in Table BCA. Results of the RTQ-PCR runs are shown in Tables BCB and BCC. Please note that CG59319-02 represents a full-length physical clone of the CG59319-01 gene, validating the prediction of the gene sequence.

Table BCA. Probe Name Ag3544

TABLE BCA
Probe Name Ag3544

| | | | Start | |
|---|---|---|---|---|
| Primers | Sequences | Length | Position | SEQ ID NO: |
| Forward | 5′-tacagatcaagcatcccaatgt-3′ | 22 | 347 | 559 |
| Probe | TET-5′-tggttaaccagcatcttagtcttctagca-3′-TAMRA | 29 | 375 | 560 |
| Reverse | 5′-ttcacgatggctttaacaaatt-3′ | 22 | 423 | 561 |

TABLE BCB
General_screening_panel_v1.4

| | Rel. | | Rel. |
|---|---|---|---|
| | Exp. (%) | | Exp. (%) |
| | Ag3544, | | Ag3544, |
| | Run | | Run |
| Tissue Name | 217048127 | Tissue Name | 217048127 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.7 |
| Hs688(B).T | | NCI-N87 | |
| Melanoma* M14 | 0.0 | Gastric ca. KATO III | 0.0 |
| Melanoma* | 0.0 | Colon ca. SW-948 | 0.2 |
| LOXIMVI | | | |
| Melanoma* SK- | 0.0 | Colon ca. SW480 | 0.0 |
| MEL-5 | | | |
| Squamous cell | 1.4 | Colon ca.* (SW480 | 0.0 |
| carcinoma SCC-4 | | met) SW620 | |
| Testis Pool | 100.0 | Colon ca. HT29 | 0.0 |
| Prostate ca.* (bone | 0.0 | Colon ca. HCT-116 | 0.3 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 1.2 | Colon ca. Colo-205 | 0.0 |
| OVCAR-3 | | | |
| Ovarian ca. SK- | 0.9 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 0.2 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.2 | Lymph Node Pool | 0.0 |
| MB-231 | | | |
| Breast ca. BT 549 | 0.0 | Fetal Skeletal Muscle | 0.0 |
| Breast ca. T47D | 0.3 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.5 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) | 0.3 |
| | | SF-539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) | 0.2 |
| | | SF-295 | |
| Lung ca. A549 | 0.0 | Brain (Amygdala) | 0.2 |
| | | Pool | |
| Lung ca. NCI-H526 | 1.0 | Brain (cerebellum) | 0.0 |
| Lung ca. NCI-H23 | 0.0 | Brain (fetal) | 0.6 |
| Lung ca. NCI-H460 | 0.0 | Brain (Hippocampus) | 0.0 |
| | | Pool | |
| Lung ca. HOP-62 | 0.0 | Cerebral Cortex Pool | 0.2 |
| Lung ca. NCI-H522 | 0.0 | Brain (Substantia | 0.0 |
| | | nigra) Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 1.6 | Salivary Gland | 0.0 |
| Renal ca. A498 | 0.0 | Thyroid (female) | 0.2 |
| Renal ca. ACHN | 0.0 | Pancreatic ca. | 0.0 |
| | | CAPAN2 | |
| Renal ca. UO-31 | 0.0 | Pancreas Pool | 0.0 |

TABLE BCC
Panel 4.1D

| | Rel. Exp. (%) | | Rel. Exp. (%) |
|---|---|---|---|
| | Ag3544, Run | | Ag3544, Run |
| Tissue Name | 169850546 | Tissue Name | 169850546 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 2.6 | Bronchial epithelium | 0.0 |
| | | TNF alpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 100.0 |
| lymphocyte act | | | |
| CD4 lymphocyte none | 0.0 | KU-812 (Basophil) | 61.1 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL- | 0.0 |
| | | 1beta | |
| Two Way MLR 7 day | 0.0 | Lung fibroblast none | 0.0 |
| PBMC rest | 0.0 | Lung fibroblast TNF | 0.0 |
| | | alpha + IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag3544 Expression of the CG59319-02 gene is low/undetectable in all samples on this panel (CTs>35). (Data not shown.)

General_screening_panel_v1.4 Summary: Ag3544 Expression of the CG59319-02 gene is restricted to a sample derived from the testis (CT=29.8). Thus, expression of this gene could be used to differentiate between this sample and other samples on this panel and as a marker of testicular tissue. Furthermore, therapeutic modulation of the expression or function of this gene may be useful in the treatment of male infertility or hypogonadism.

Panel 4.1D Summary: Ag3544 Expression of the CG59319-02 gene is restricted to samples derived from the basophil cell line KU-812 (CTs=32). Thus, expression of this gene could be used as a marker of this cell type. Furthermore, the specific pattern of expression of this gene suggests that therapeutic modulation of the expression or function of the protein encoded by this gene may block or inhibit inflammation or tissue damage due to basophil activation in response to asthma, allergies, hypersensitivity reactions, psoriasis, and viral infections.

BD. CG59576-01: Olfactory Receptor

Expression of gene CG59576-01 was assessed using the primer-probe set Ag3478, described in Table BDA. Results of the RTQ-PCR runs are shown in Table BDB.

Table BDA. Probe Name Ag3478

TABLE BDA
Probe Name Ag3478

| | | | Start | SEQ ID |
|---|---|---|---|---|
| Primers | Sequences | Length | Position | NO: |
| Forward | 5′-tggaagtgtagccctgatgtac-3′ | 22 | 708 | 562 |
| Probe | TET-5′-tgctcttctctgccaagtactccttt-3′-TAMRA | 26 | 731 | 563 |
| Reverse | 5′-aaacattaggctgatggttgtg-3′ | 22 | 765 | 564 |

TABLE BDB
Panel 4D

| | Rel. Exp. (%) | | Rel. Exp. (%) |
|---|---|---|---|
| | Ag3478, Run | | Ag3478, Run |
| Tissue Name | 166441540 | Tissue Name | 166441540 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 9.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 4.6 |
| | | PMA/ionomycin | |
| 2ry Th1/Th2/Tr1_anti- | 2.6 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 25.7 |
| CD40 | | | |
| Monocytes rest | 0.0 | IBD Crohn's | 24.3 |
| Monocytes LPS | 0.0 | Colon | 5.9 |
| Macrophages rest | 0.0 | Lung | 5.8 |
| Macrophages LPS | 0.0 | Thymus | 5.9 |
| HUVEC none | 0.0 | Kidney | 10.2 |
| HUVEC starved | 0.0 | | |

CNS_neurodegeneration_v1.0 Summary: Ag3478 Expression of the CG59576-01 gene is low/undetectable in all samples on this panel (CTs>35). (Data not shown.)

General_screening_panel_v1.4 Summary: Ag3478 Expression of the CG59576-01 gene is low/undetectable in all samples on this panel (CTs>35). (Data not shown.)

General_screening_panel_v1.5 Summary: Ag3478 Expression of the CG59576-01 gene is low/undetectable in all samples on this panel (CTs>35). (Data not shown.)

Panel 4D Summary: Ag3478 Expression of the CG59576-01 gene is restricted to a sample derived from liver cirrhosis (CT=32.3). Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

Panel 5 Islet Summary: Ag3478 Expression of the CG59576-01 gene is low/undetectable in all samples on this panel (CTs>35). (Data not shown.)

BE. CG59557-01: Olfactory Receptor

Expression of gene CG59557-01 was assessed using the primer-probe set Ag3470, described in Table BEA. Results of the RTQ-PCR runs are shown in Table BEB.

Table BEA. Probe Name Ag3470

TABLE BEA
Probe Name Ag3470

| | | | Start | SEQ ID |
|---|---|---|---|---|
| Primers | Sequences | Length | Position | NO: |
| Forward | 5′-ccaaccttctcagtgaacagaa-3′ | 22 | 413 | 565 |
| Probe | TET-5′-tctctttcataggttgcctcctgca-3′-TAMRA | 27 | 440 | 566 |
| Reverse | 5′-ccgagtgagtggaagaagtaca-3′ | 22 | 467 | 567 |

TABLE BEB
Panel 4D

| | Rel. Exp. (%) | | Rel. Exp. (%) |
|---|---|---|---|
| | Ag3470, Run | | Ag3470, Run |
| Tissue Name | 166417125 | Tissue Name | 166417125 |
| Secondary Th1 act | 0.0 | HUVEC IL-1beta | 0.0 |
| Secondary Th2 act | 3.2 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 3.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 2.8 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 2.9 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 2.2 |
| LAK cells IL-2 + IFN | 0.0 | NCI-H292 none | 0.0 |
| gamma | | | |
| LAK cells IL-2 + IL-18 | 3.2 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 5.8 | Lung fibroblast none | 0.0 |
| PBMC PWM | 2.7 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 6.5 | Lung fibroblast IFN | 0.0 |
| | | gamma | |
| B lymphocytes CD40L | 2.2 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 3.2 | Dermal fibroblast IFN | 0.0 |
| | | gamma | |
| Dendritic cells LPS | 20.9 | Dermal fibroblast IL-4 | 0.0 |
| Dendritic cells anti- | 0.0 | IBD Colitis 2 | 19.6 |
| CD40 | | | |
| Monocytes rest | 0.0 | IBD Crohn's | 4.9 |
| Monocytes LPS | 6.3 | Colon | 13.9 |
| Macrophages rest | 21.6 | Lung | 14.8 |
| Macrophages LPS | 0.0 | Thymus | 2.2 |
| HUVEC none | 0.0 | Kidney | 0.0 |
| HUVEC starved | 0.0 | | |

CNS_neurodegeneration_v1.0 Summary: Ag3470 Expression of the CG59557-01 gene is low/undetectable in all samples on this panel (CTs>35). (Data not shown.)

General_screening_panel_v1.4 Summary: Ag3470 Expression of the CG59557-01 gene is low/undetectable in all samples on this panel (CTs>35). (Data not shown.)

Panel 4D Summary: Ag3470 Expression of the CG59557-01 gene is detected in a liver cirrhosis sample (CT=32.2). Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

BF. CG59555-01: Olfactory Receptor

Expression of gene CG59555-01 was assessed using the primer-probe set Ag3467, described in Table BFA. Results of the RTQ-PCR runs are shown in Tables BFB, BFC and BFD.

Table BFA. Probe Name Ag3467

TABLE BFA
Probe Name Ag3467

| | | | Start | SEQ ID |
|---|---|---|---|---|
| Primers | Sequences | Length | Position | NO: |
| Forward | 5′-ggcaaggaaagtcattcctaa-3′ | 21 | 953 | 568 |
| Probe | TET-5′-tggtgtgacatttgactctccctcct-3′-TAMRA | 26 | 975 | 569 |
| Reverse | 5′-tggtaccaagattccaggagat-3′ | 22 | 1006 | 570 |

TABLE BFB
CNS_neurodegeneration_v1.0

| | Rel. | | Rel. |
|---|---|---|---|
| | Exp. (%) | | Exp. (%) |
| | Ag3467, | | Ag3467, |
| | Run | | Run |
| Tissue Name | 210376517 | Tissue Name | 210376517 |
| AD 1 Hippo | 4.6 | Control (Path) 3 | 14.8 |
| | | Temporal Ctx | |
| AD 2 Hippo | 29.7 | Control (Path) 4 | 15.2 |
| | | Temporal Ctx | |
| AD 3 Hippo | 10.7 | AD 1 Occipital | 14.5 |
| | | Ctx | |
| AD 4 Hippo | 28.9 | AD 2 Occipital | 0.0 |
| | | Ctx (Missing) | |
| AD 5 hippo | 21.6 | AD 3 Occipital | 14.5 |
| | | Ctx | |
| AD 6 Hippo | 100.0 | AD 4 Occipital | 14.8 |
| | | Ctx | |
| Control 2 Hippo | 8.8 | AD 5 Occipital | 13.7 |
| | | Ctx | |
| Control 4 Hippo | 35.6 | AD 6 Occipital | 10.3 |
| | | Ctx | |
| Control (Path) 3 | 21.9 | Control 1 Occipital | 18.3 |
| Hippo | | Ctx | |
| AD 1 Temporal Ctx | 28.3 | Control 2 Occipital | 7.9 |
| | | Ctx | |
| AD 2 Temporal Ctx | 37.4 | Control 3 Occipital | 16.2 |
| | | Ctx | |
| AD 3 Temporal Ctx | 7.4 | Control 4 Occipital | 24.0 |
| | | Ctx | |
| AD 4 Temporal Ctx | 28.3 | Control (Path) 1 | 28.3 |
| | | Occipital Ctx | |
| AD 5 Inf Temporal | 19.3 | Control (Path) 2 | 10.1 |
| Ctx | | Occipital Ctx | |
| AD 5 Sup Temporal | 33.4 | Control (Path) 3 | 12.6 |
| Ctx | | Occipital Ctx | |
| AD 6 Inf Temporal | 39.8 | Control (Path) 4 | 14.3 |
| Ctx | | Occipital Ctx | |
| AD 6 Sup Temporal | 83.5 | Control 1 Parietal | 8.7 |
| Ctx | | Ctx | |
| Control 1 Temporal | 14.4 | Control 2 Parietal | 22.2 |
| Ctx | | Ctx | |
| Control 2 Temporal | 13.6 | Control 3 Parietal | 9.8 |
| Ctx | | Ctx | |
| Control 3 Temporal | 11.8 | Control (Path) 1 | 33.2 |
| Ctx | | Parietal Ctx | |
| Control 4 Temporal | 16.0 | Control (Path) 2 | 12.4 |
| Ctx | | Parietal Ctx | |
| Control (Path) 1 | 24.3 | Control (Path) 3 | 19.6 |
| Temporal Ctx | | Parietal Ctx | |
| Control (Path) 2 | 6.1 | Control (Path) 4 | 12.9 |
| Temporal Ctx | | Parietal Ctx | |

TABLE BFC
General_screening_panel_v1.4

| | Rel. | | Rel. |
|---|---|---|---|
| | Exp. (%) | | Exp. (%) |
| | Ag3467, | | Ag3467, |
| | Run | | Run |
| Tissue Name | 217119371 | Tissue Name | 217119371 |
| Adipose | 11.6 | Renal ca. TK-10 | 14.6 |
| Melanoma* | 27.0 | Bladder | 14.1 |
| Hs688(A).T | | | |
| Melanoma* | 27.9 | Gastric ca. (liver met.) | 3.2 |
| Hs688(B).T | | NCI-N87 | |
| Melanoma* M14 | 11.2 | Gastric ca. KATO III | 2.1 |
| Melanoma* | 0.0 | Colon ca. SW-948 | 0.4 |
| LOXIMVI | | | |
| Melanoma* SK- | 0.0 | Colon ca. SW480 | 12.7 |
| MEL-5 | | | |
| Squamous cell | 1.8 | Colon ca.* (SW480 | 10.4 |
| carcinoma SCC-4 | | met) SW620 | |
| Testis Pool | 7.0 | Colon ca. HT29 | 1.7 |
| Prostate ca.* (bone | 15.2 | Colon ca. HCT-116 | 7.9 |
| met) PC-3 | | | |
| Prostate Pool | 9.7 | Colon ca. CaCo-2 | 5.3 |
| Placenta | 1.7 | Colon cancer tissue | 3.6 |
| Uterus Pool | 5.1 | Colon ca. SW1116 | 0.4 |
| Ovarian ca. | 4.1 | Colon ca. Colo-205 | 0.2 |
| OVCAR-3 | | | |
| Ovarian ca. SK- | 16.2 | Colon ca. SW-48 | 0.5 |
| OV-3 | | | |
| Ovarian ca. | 5.2 | Colon Pool | 20.6 |
| OVCAR-4 | | | |
| Ovarian ca. | 13.9 | Small Intestine Pool | 26.8 |
| OVCAR-5 | | | |
| Ovarian ca. | 0.0 | Stomach Pool | 19.5 |
| IGROV-1 | | | |
| Ovarian ca. | 9.5 | Bone Marrow Pool | 16.7 |
| OVCAR-8 | | | |
| Ovary | 8.4 | Fetal Heart | 23.5 |
| Breast ca. MCF-7 | 0.8 | Heart Pool | 11.2 |
| Breast ca. MDA- | 26.2 | Lymph Node Pool | 31.9 |
| MB-231 | | | |
| Breast ca. BT 549 | 0.0 | Fetal Skeletal Muscle | 7.1 |
| Breast ca. T47D | 18.2 | Skeletal Muscle Pool | 1.8 |
| Breast ca. MDA-N | 10.9 | Spleen Pool | 22.4 |
| Breast Pool | 26.6 | Thymus Pool | 25.9 |
| Trachea | 9.5 | CNS cancer | 0.4 |
| | | (glio/astro) U87-MG | |
| Lung | 12.4 | CNS cancer | 0.2 |
| | | (glio/astro) U-118-MG | |
| Fetal Lung | 100.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 11.6 | CNS cancer (astro) | 1.5 |
| | | SNB-75 | |
| Lung ca. NCI-H146 | 0.0 | CNS cancer (glio) | 1.1 |
| | | SNB-19 | |
| Lung ca. SHP-77 | 0.1 | CNS cancer (glio) | 20.0 |
| | | SF-295 | |
| Lung ca. A549 | 3.0 | Brain (Amygdala) | 1.9 |
| | | Pool | |
| Lung ca. NCI-H526 | 0.0 | Brain (cerebellum) | 1.0 |
| Lung ca. NCI-H23 | 13.9 | Brain (fetal) | 2.5 |
| Lung ca. NCI-H460 | 5.4 | Brain (Hippocampus) | 0.2 |
| | | Pool | |
| Lung ca. HOP-62 | 6.7 | Cerebral Cortex Pool | 1.3 |
| Lung ca. NCI-H522 | 0.0 | Brain (Substantia | 1.6 |
| | | nigra) Pool | |
| Liver | 0.1 | Brain (Thalamus) Pool | 2.5 |
| Fetal Liver | 6.6 | Brain (whole) | 1.1 |
| Liver ca. HepG2 | 1.4 | Spinal Cord Pool | 4.5 |
| Kidney Pool | 34.4 | Adrenal Gland | 6.3 |
| Fetal Kidney | 76.3 | Pituitary gland Pool | 4.5 |
| Renal ca. 786-0 | 28.1 | Salivary Gland | 1.8 |
| Renal ca. A498 | 12.1 | Thyroid (female) | 13.4 |
| Renal ca. ACHN | 23.0 | Pancreatic ca. | 1.0 |
| | | CAPAN2 | |
| Renal ca. UO-31 | 25.0 | Pancreas Pool | 27.2 |

TABLE BFD
Panel 4D

| | Rel. Exp. (%) | | Rel. Exp. (%) |
|---|---|---|---|
| | Ag3467, Run | | Ag3467, Run |
| Tissue Name | 166417105 | Tissue Name | 166417105 |
| Secondary Th1 act | 2.4 | HUVEC IL-1beta | 1.5 |
| Secondary Th2 act | 5.1 | HUVEC IFN gamma | 18.2 |
| Secondary Tr1 act | 7.2 | HUVEC TNF alpha + | 6.2 |
| | | IFN gamma | |
| Secondary Th1 rest | 18.2 | HUVEC TNF alpha + | 1.7 |
| | | IL4 | |
| Secondary Th2 rest | 14.6 | HUVEC IL-11 | 1.8 |
| Secondary Tr1 rest | 22.1 | Lung Microvascular EC | 0.6 |
| | | none | |
| Primary Th1 act | 1.3 | Lung Microvascular EC | 0.3 |
| | | TNF alpha + IL-1beta | |
| Primary Th2 act | 9.0 | Microvascular Dermal | 0.1 |
| | | EC none | |
| Primary Tr1 act | 7.2 | Microsvasular Dermal | 0.5 |
| | | EC TNF alpha + IL-1beta | |
| Primary Th1 rest | 100.0 | Bronchial epithelium | 1.5 |
| | | TNF alpha + IL1beta | |
| Primary Th2 rest | 38.7 | Small airway epithelium | 1.6 |
| | | none | |
| Primary Tr1 rest | 28.1 | Small airway epithelium | 6.6 |
| | | TNF alpha + IL-1beta | |
| CD45RA CD4 | 1.9 | Coronery artery SMC rest | 4.5 |
| lymphocyte act | | | |
| CD45RO CD4 | 8.7 | Coronery artery SMC | 5.1 |
| lymphocyte act | | TNF alpha + IL-1beta | |
| CD8 lymphocyte act | 4.0 | Astrocytes rest | 0.4 |
| Secondary CD8 | 7.5 | Astrocytes TNF alpha + | 1.7 |
| lymphocyte rest | | IL-1beta | |
| Secondary CD8 | 4.7 | KU-812 (Basophil) rest | 0.7 |
| lymphocyte act | | | |
| CD4 lymphocyte none | 9.3 | KU-812 (Basophil) | 3.2 |
| | | PMA/ionomycin | |
| 2ry Th1/Th2/Tr1_anti- | 52.5 | CCD1106 | 1.5 |
| CD95 CH11 | | (Keratinocytes) none | |
| LAK cells rest | 4.6 | CCD1106 | 16.4 |
| | | (Keratinocytes) | |
| | | TNF alpha + IL-1beta | |
| LAK cells IL-2 | 15.1 | Liver cirrhosis | 14.6 |
| LAK cells IL-2 + IL-12 | 6.5 | Lupus kidney | 37.6 |
| LAK cells IL-2 + IFN | 10.9 | NCI-H292 none | 4.6 |
| gamma | | | |
| LAK cells IL-2 + IL-18 | 6.9 | NCI-H292 IL-4 | 5.2 |
| LAK cells | 1.1 | NCI-H292 IL-9 | 5.6 |
| PMA/ionomycin | | | |
| NK Cells IL-2 rest | 3.1 | NCI-H292 IL-13 | 2.3 |
| Two Way MLR 3 day | 11.0 | NCI-H292 IFN gamma | 1.5 |
| Two Way MLR 5 day | 6.0 | HPAEC none | 3.6 |
| Two Way MLR 7 day | 6.7 | HPAEC TNF alpha + IL- | 10.5 |
| | | 1beta | |
| PBMC rest | 2.6 | Lung fibroblast none | 15.3 |
| PBMC PWM | 4.2 | Lung fibroblast TNF | 5.9 |
| | | alpha + IL-1beta | |
| PBMC PHA-L | 3.1 | Lung fibroblast IL-4 | 5.6 |
| Ramos (B cell) none | 0.0 | Lung fibroblast IL-9 | 5.6 |
| Ramos (B cell) | 0.1 | Lung fibroblast IL-13 | 5.1 |
| ionomycin | | | |
| B lymphocytes PWM | 6.0 | Lung fibroblast IFN | 7.9 |
| | | gamma | |
| B lymphocytes CD40L | 6.5 | Dermal fibroblast | 4.5 |
| and IL-4 | | CCD1070 rest | |
| EOL-1 dbcAMP | 0.0 | Dermal fibroblast | 21.6 |
| | | CCD1070 TNF alpha | |
| EOL-1 dbcAMP | 0.0 | Dermal fibroblast | 2.0 |
| PMA/ionomycin | | CCD1070 IL-1beta | |
| Dendritic cells none | 0.4 | Dermal fibroblast IFN | 3.5 |
| | | gamma | |
| Dendritic cells LPS | 0.1 | Dermal fibroblast IL-4 | 6.9 |
| Dendritic cells anti- | 0.1 | IBD Colitis 2 | 10.6 |
| CD40 | | | |
| Monocytes rest | 1.6 | IBD Crohn's | 1.8 |
| Monocytes LPS | 1.7 | Colon | 34.9 |
| Macrophages rest | 8.4 | Lung | 6.7 |
| Macrophages LPS | 1.2 | Thymus | 26.8 |
| HUVEC none | 2.6 | Kidney | 11.3 |
| HUVEC starved | 2.8 | | |

CNS_neurodegeneration_v1.0 Summary: Ag3467 The CG59555-01 gene encodes a putative GPCR. It is expressed at low to moderate levels in most of the samples used in this panel. This panel confirms the expression of CG59555-01 gene at low levels in the brains of an independent group of individuals. However, no differential expression of this gene was detected between Alzheimer's diseased postmortem brains and those of non-demented controls in this experiment. Please see Panel 10.4 for a discussion of the potential utility of this gene in treatment of central nervous system disorders.

General_screening_panel_v1.4 Summary: Ag3467 The CG59555-01 gene encodes a putative GPCR. It is expressed at low to moderate levels in large number of the samples used in this panel. Highest expression of this gene is detected in fetal lung (CT=28). Interestingly, this gene is expressed at much higher levels in fetal (CT=28) when compared to adult lung (CT=31). Therefore, expression of this gene can be used to distinguish fetal lung from adult lung and from other samples used in this panel. In addition, this gene is also expressed at much higher levels in fetal fetal liver (CT=32) as compared to adult liver (CT=38). Thus, expression of this gene can be used to distinguish fetal liver from adult liver.

Among tissues with metabolic or endocrine function, this gene is expressed at low to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases;, such as obesity and diabetes.

This gene is also expressed at low levels in all regions of the central nervous system examined, including amygdala, substantia nigra, thalamus, cerebellum, cerebral cortex, and spinal cord. Several neurotransmitter receptors are GPCRs, including the dopamine receptor family, the serotonin receptor family, the GABAB receptor, muscarinic acetylcholine receptors, and others; thus this GPCR may represent a novel neurotransmitter receptor. Targeting various neurotransmitter receptors (dopamine, serotonin) has proven to be an effective therapy in psychiatric illnesses such as schizophrenia, bipolar disorder, and depression. Furthermore, the cerebral cortex and hippocampus are regions of the brain that are known to be involved in Alzheimer's disease, seizure disorders, and in the normal process of memory formation. Therefore, this gene may play a role in central nervous system disorders such as Alzheimer's disease, Parkinson's disease, epilepsy, multiple sclerosis, schizophrenia and depression.

Panel 4D Summary: Ag3467 The CG59555-01 gene encodes a putative GPCR. Highest expression of this gene is detected in resting primary Th1 cells (CT=27). This gene is expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests; that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

BG. CG59551-01: Olfactory Receptor

Expression of gene CG59551-01 was assessed using the primer-probe set Ag3463, described in Table BGA. Results of the RTQ-PCR runs are shown in Tables BGB and BGC.

Table BGA. Probe Name Ag3463

TABLE BGA
Probe Name Ag3463

| | | | Start | SEQ ID |
|---|---|---|---|---|
| Primers | Sequences | Length | Position | NO: |
| Forward | 5′-ctatggtttccagatgtttcca-3′ | 22 | 78 | 571 |
| Probe | 5′-tagatgttccagctgcccatctctga-3′-TAMRA | 26 | 105 | 572 |
| Reverse | 5′-attqtqaqacacaqctqqattt-3′ | 22 | 132 | 573 |

TABLE BGB
General_screening_panel_v1.4

| | Rel. | | Rel. |
|---|---|---|---|
| | Exp. (%) | | Exp. (%) |
| | Ag3463, | | Ag3463, |
| | Run | | Run |
| Tissue Name | 217067349 | Tissue Name | 217067349 |
| Adipose | 0.0 | Renal ca. TK-10 | 9.0 |
| Melanoma* | 0.0 | Bladder | 11.2 |
| Hs688(A).T | | | |
| Melanoma* | 0.0 | Gastric ca. (liver met.) | 13.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 43.8 | Colon ca. SW480 | 11.6 |
| MEL-5 | | | |
| Squamous cell | 0.0 | Colon ca.* (SW480 | 0.0 |
| carcinoma SCC-4 | | met) SW620 | |
| Testis Pool | 85.9 | Colon ca. HT29 | 0.0 |
| Prostate ca.* (bone | 0.0 | Colon ca. HCT-116 | 10.7 |
| met) PC-3 | | | |
| Prostate Pool | 7.2 | Colon ca. CaCo-2 | 12.8 |
| Placenta | 12.8 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 100.0 | Colon ca. SW-48 | 0.0 |
| OV-3 | | | |
| Ovarian ca. | 11.9 | Colon Pool | 12.5 |
| OVCAR-4 | | | |
| Ovarian ca. | 11.3 | Small Intestine Pool | 0.0 |
| OVCAR-5 | | | |
| Ovarian ca. | 26.1 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 23.2 |
| MB-231 | | | |
| Breast ca. BT 549 | 20.3 | Fetal Skeletal Muscle | 82.9 |
| Breast ca. T47D | 0.0 | Skeletal Muscle Pool | 0.0 |
| Breast ca. MDA-N | 31.9 | Spleen Pool | 0.0 |
| Breast Pool | 12.5 | Thymus Pool | 0.0 |
| Trachea | 20.9 | CNS cancer | 0.0 |
| | | (glio/astro) U87-MG | |
| Lung | 0.0 | CNS cancer | 24.0 |
| | | (glio/astro) U-118-MG | |
| Fetal Lung | 23.7 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 38.2 |
| | | SNB-75 | |
| Lung ca. NCI-H146 | 0.0 | CNS cancer (glio) | 0.0 |
| | | SNB-19 | |
| Lung ca. SHP-77 | 8.8 | CNS cancer (glio) | 33.2 |
| | | SF-295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 57.4 | Brain (fetal) | 48.6 |
| Lung ca. NCI-H460 | 31.2 | Brain (Hippocampus) | 21.3 |
| | | Pool | |
| Lung ca. HOP-62 | 0.0 | Cerebral Cortex Pool | 35.6 |
| Lung ca. NCI-H522 | 14.4 | Brain (Substantia | 15.5 |
| | | nigra) Pool | |
| Liver | 0.0 | Brain (Thalamus) Pool | 14.1 |
| Fetal Liver | 0.0 | Brain (whole) | 0.0 |
| Liver ca. HepG2 | 5.0 | Spinal Cord Pool | 29.5 |
| Kidney Pool | 37.9 | Adrenal Gland | 14.8 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 12.2 |

TABLE BGC
Panel 4.1D

| | Rel. Exp. (%) | | Rel. Exp. (%) |
|---|---|---|---|
| | Ag3463, Run | | Ag3463, Run |
| Tissue Name | 169839351 | Tissue Name | 169839351 |
| Secondary Th1 act | 0.0 | HUVEC IL-1beta | 0.0 |
| Secondary Th2 act | 0.0 | HUVEC IFN gamma | 0.0 |
| Secondary Tr1 act | 1.3 | HUVEC TNF alpha + | 0.0 |
| | | IFN gamma | |
| Secondary Th1 rest | 2.6 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 1.4 |
| | | EC TNF alpha + IL-1beta | |
| Primary Th1 rest | 0.0 | Bronchial epithelium | 0.6 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 7.2 |
| lymphocyte act | | | |
| CD4 lymphocyte none | 0.0 | KU-812 (Basophil) | 100.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 1.1 |
| | | (Keratinocytes) | |
| | | TNF alpha + IL-1beta | |
| LAK cells IL-2 | 0.0 | Liver cirrhosis | 1.2 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 1.3 | HPAEC none | 1.2 |
| Two Way MLR 5 day | 0.0 | HPAEC TNF alpha + IL- | 0.0 |
| | | 1beta | |
| Two Way MLR 7 day | 0.0 | Lung fibroblast none | 1.0 |
| PBMC rest | 0.0 | Lung fibroblast TNF | 0.0 |
| | | alpha + IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 1.3 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 1.2 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 6.1 | Dermal fibroblast IL-4 | 0.0 |
| Dendritic cells LPS | 0.0 | Dermal Fibroblast rest | 0.0 |
| Dendritic cells anti- | 1.2 | Neutrophils TNFa + LPS | 0.0 |
| CD40 | | | |
| Monocytes rest | 0.0 | Neutrophils rest | 0.0 |
| Monocytes LPS | 0.0 | Colon | 0.5 |
| Macrophages rest | 4.1 | Lung | 0.0 |
| Macrophages LPS | 0.0 | Thymus | 0.0 |
| HUVEC none | 0.0 | Kidney | 2.3 |
| HUVEC starved | 0.0 | | |

CNS_neurodegeneration_v1.0 Summary: Ag3463

Expression of the CG59551-01 gene is low/undetectable in all the samples on this panel. (Data not shown.)

General_screening_panel_v1.4 Summary: Ag3463 The CG59551-01 gene encodes a putative GPCR. Highest expression of this gene is detected in an ovarian cancer cell line SK-OV-3 (CT=34). In addition, low expression of this gene is also observed in fetal skeletal muscle (CT=34.4), one of the lung cancer cell line (CT=34.9), and testis (CT=34.3). Thus, expression of this gene can be used to distinguish these sample from other samples used in this panel. In addition, therapeutic modulation of the activity of the GPCR encoded by this gene may be useful in the treatment of ovarian and lung cancer, fertility, hypogonadism, and muscle related diseases.

Panel 4.1D Summary: Ag3463 The CG59551-01 gene encodes a putative GPCR. Highest expression of this gene is seen in KU-812 cells treated with PMA/ionomycin (CT=30.86). Thus, expression of this gene can be used to distinguish this sample from other samples used in this panel. In addition, expression of this gene is high in KU-812 (basophils) cells treated with PMA/ionomycin (CT=30.86) as compared to resting KU-812 cells (CT=34.66). Therefore, expression of this gene can be used to distinguish resting from PMA/ionomycin treated-basophils. It is known that GPCR-type receptors are important in multiple physiological responses mediated by basophils (ref. 1). Therefore, antibody or small molecule therapies designed with the protein encoded for by this gene could block or inhibit inflammation or tissue damage due to basophil activation in response to asthma, allergies, hypersensitivity reactions, psoriasis, and viral infections.

### REFERENCES

1. Heinemann A., Hartnell A., Stubbs V. E., Murakami K., Soler D., LaRosa G., Askenase P. W., Williams T. J., Sabroe I. (2000) Basophil responses to chemokines are regulated by both sequential and cooperative receptor signaling. J. Immunol. 165: 7224-7233.

BH. CG759540-01: Olfactory Receptor

Expression of gene CG59540-01 was assessed using the primer-probe sets A03460 and Ag 1519, described in Tables BHA and BHB. Results of the RTQ-PCR runs are shown in Tables BHC, BHD and BHE.

Table BHA. Probe Name Ag3460

TABLE BHA
Probe Name Ag3460

| | | | Start | SEQ ID |
|---|---|---|---|---|
| Primers | Sequences | Length | Position | NO: |
| Forward | 5′-tcagtgcagagatggagatctt-3′ | 22 | 97 | 574 |
| Probe | TET-5′-tgcatcttctccctgttatatctcttca-3′-TAMRA | 28 | 126 | 575 |
| Reverse | 5′-gacagatgagtcccatgttcat-3′ | 22 | 171 | 576 |

Table BHB. Probe Name Ag1519

TABLE BHB
Probe Name Ag1519

| | | | Start | SEQ ID |
|---|---|---|---|---|
| Primers | Sequences | Length | Position | NO: |
| Forward | 5′-cctggccctcataaatctaatt-3′ | 22 | 503 | 577 |
| Probe | TET-5′-ctccttctaaggctgcccttctgtgg-3′-TAMRA | 26 | 525 | 578 |
| Reverse | 5′-acagacagaatttcaccgaaga-3′ | 22 | 571 | 579 |

TABLE BHC
Panel 1.2

| | Rel. | | Rel. |
|---|---|---|---|
| | Exp. (%) | | Exp. (%) |
| | Ag1519, | | Ag1519, |
| | Run | | Run |
| Tissue Name | 142098791 | Tissue Name | 142098791 |
| Endothelial cells | 0.0 | Renal ca. 786-0 | 32.1 |
| Heart (Fetal) | 1.3 | Renal ca. A498 | 10.7 |
| Pancreas | 1.5 | Renal ca. RXF 393 | 7.5 |
| Pancreatic ca. | 3.6 | Renal ca. ACHN | 10.2 |
| CAPAN 2 | | | |
| Adrenal Gland | 4.7 | Renal ca. UO-31 | 26.8 |
| Thyroid | 0.4 | Renal ca. TK-10 | 14.0 |
| Salivary gland | 27.7 | Liver | 7.2 |
| Pituitary gland | 0.0 | Liver (fetal) | 3.5 |
| Brain (fetal) | 0.0 | Liver ca. | 0.9 |
| | | (hepatoblast) HepG2 | |
| Brain (whole) | 0.0 | Lung | 0.0 |
| Brain (amygdala) | 0.0 | Lung (fetal) | 1.3 |
| Brain (cerebellum) | 0.0 | Lung ca. (small cell) | 22.8 |
| | | LX-1 | |
| Brain | 0.3 | Lung ca. (small cell) | 11.5 |
| (hippocampus) | | NCI-H69 | |
| Brain (thalamus) | 0.2 | Lung ca. (s. cell var.) | 0.0 |
| | | SHP-77 | |
| Cerebral Cortex | 0.3 | Lung ca. (large | 2.3 |
| | | cell)NCI-H460 | |
| Spinal cord | 0.0 | Lung ca. (non-sm. | 5.1 |
| | | cell) A549 | |
| glio/astro U87-MG | 0.4 | Lung ca. (non-s. cell) | 7.7 |
| | | NCI-H23 | |
| glio/astro | 0.9 | Lung ca. (non-s. cell) | 6.8 |
| U-118-MG | | HOP-62 | |
| astrocytoma | 0.0 | Lung ca. (non-s. cl) | 0.9 |
| SW1783 | | NCI-H522 | |
| neuro*; met SK-N- | 0.0 | Lung ca. (squam.) | 52.1 |
| AS | | SW 900 | |
| astrocytoma SF-539 | 0.0 | Lung ca. (squam.) | 4.1 |
| | | NCI-H596 | |
| astrocytoma | 0.0 | Mammary gland | 11.8 |
| SNB-75 | | | |
| glioma SNB-19 | 4.0 | Breast ca.* (pl. ef) | 11.3 |
| | | MCF-7 | |
| glioma U251 | 0.0 | Breast ca.* (pl. ef) | 1.4 |
| | | MDA-MB-231 | |
| glioma SF-295 | 2.8 | Breast ca.* (pl. ef) | 6.3 |
| | | T47D | |
| Heart | 13.9 | Breast ca. BT-549 | 0.0 |
| Skeletal Muscle | 0.2 | Breast ca. MDA-N | 12.5 |
| Bone marrow | 0.7 | Ovary | 1.5 |
| Thymus | 0.0 | Ovarian ca. | 12.4 |
| | | OVCAR-3 | |
| Spleen | 0.7 | Ovarian ca. | 23.8 |
| | | OVCAR-4 | |
| Lymph node | 0.0 | Ovarian ca. | 38.2 |
| | | OVCAR-5 | |
| Colorectal Tissue | 4.5 | Ovarian ca. | 35.6 |
| | | OVCAR-8 | |
| Stomach | 2.6 | Ovarian ca. IGROV-1 | 2.4 |
| Small intestine | 2.6 | Ovarian ca. (ascites) | 11.2 |
| | | SK-OV-3 | |
| Colon ca. SW480 | 3.1 | Uterus | 1.7 |
| Colon ca.* SW620 | 12.3 | Placenta | 0.8 |
| (SW480 met) | | | |
| Colon ca. HT29 | 12.3 | Prostate | 14.2 |
| Colon ca. HCT-116 | 14.3 | Prostate ca.* (bone | 12.6 |
| | | met) PC-3 | |
| Colon ca. CaCo-2 | 11.5 | Testis | 0.4 |
| Colon ca. Tissue | 5.7 | Melanoma | 6.5 |
| (ODO3866) | | Hs688(A).T | |
| Colon ca. | 100.0 | Melanoma* (met) | 12.2 |
| HCC-2998 | | Hs688(B).T | |
| Gastric ca.* (liver | 15.7 | Melanoma UACC- | 0.0 |
| met) NCI-N87 | | 62 | |
| Bladder | 95.3 | Melanoma M14 | 10.3 |
| Trachea | 1.0 | Melanoma LOX | 0.0 |
| | | IMVI | |
| Kidney | 55.9 | Melanoma* (met) | 0.0 |
| | | SK-MEL-5 | |
| Kidney (fetal) | 7.7 | | |

TABLE BHD
Panel 1.3D

| | Rel. Exp. (%) | | Rel. Exp. (%) |
|---|---|---|---|
| | Ag1519, | | Ag1519, |
| Tissue Name | Run 165529518 | Tissue Name | Run 165529518 |
| Liver adenocarcinoma | 0.0 | Kidney (fetal) | 0.0 |
| Pancreas | 38.7 | Renal ca. 786-0 | 8.1 |
| Pancreatic ca. CAPAN 2 | 7.9 | Renal ca. A498 | 0.0 |
| Adrenal gland | 29.9 | Renal ca. RXF 393 | 29.7 |
| Thyroid | 26.6 | Renal ca. ACHN | 13.4 |
| Salivary gland | 0.0 | Renal ca. UO-31 | 0.0 |
| Pituitary gland | 17.2 | Renal ca. TK-10 | 16.8 |
| Brain (fetal) | 0.0 | Liver | 0.0 |
| Brain (whole) | 0.0 | Liver (fetal) | 27.4 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 15.6 |
| Brain (substantia nigra) | 0.0 | Lung ca. (small cell) | 50.7 |
| | | LX-1 | |
| Brain (thalamus) | 0.0 | Lung ca. (small cell) | 0.0 |
| | | NCI-H69 | |
| Cerebral Cortex | 0.0 | Lung ca. (s. cell var.) | 25.0 |
| | | SHP-77 | |
| Spinal cord | 0.0 | Lung ca. (large | 26.1 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s. cell) | 0.0 |
| | | NCI-H23 | |
| astrocytoma SW1783 | 0.0 | Lung ca. (non-s. cell) | 27.4 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 18.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 27.0 |
| glioma U251 | 18.8 | Breast ca.* (pl. ef) | 27.0 |
| | | MCF-7 | |
| glioma SF-295 | 0.0 | Breast ca.* (pl. ef) | 45.7 |
| | | MDA-MB-231 | |
| Heart (fetal) | 16.4 | Breast ca.* (pl. ef) | 13.7 |
| | | T47D | |
| Heart | 0.0 | Breast ca. BT-549 | 0.0 |
| Skeletal muscle (fetal) | 0.0 | Breast ca. MDA-N | 13.8 |
| Skeletal muscle | 18.8 | Ovary | 0.0 |
| Bone marrow | 0.0 | Ovarian ca. | 0.0 |
| | | OVCAR-3 | |
| Thymus | 0.0 | Ovarian ca. | 11.4 |
| | | OVCAR-4 | |
| Spleen | 0.0 | Ovarian ca. | 2.6 |
| | | OVCAR-5 | |
| Lymph node | 34.4 | Ovarian ca. | 12.3 |
| | | OVCAR-8 | |
| Colorectal | 100.0 | Ovarian ca. IGROV-1 | 0.0 |
| Stomach | 0.0 | Ovarian ca.* | 33.7 |
| | | (ascites) SK-OV-3 | |
| Small intestine | 0.0 | Uterus | 0.0 |
| Colon ca. SW480 | 22.8 | Placenta | 17.6 |
| Colon ca.* | 10.0 | Prostate | 0.0 |
| SW620(SW480 met) | | | |
| Colon ca. HT29 | 16.7 | Prostate ca.* (bone | 0.0 |
| | | met)PC-3 | |
| Colon ca. HCT-116 | 16.8 | Testis | 0.0 |
| Colon ca. CaCo-2 | 15.6 | Melanoma | 15.1 |
| | | Hs688(A).T | |
| Colon ca. | 28.9 | Melanoma* (met) | 9.0 |
| tissue(ODO3866) | | Hs688(B).T | |
| Colon ca. HCC-2998 | 31.0 | Melanoma UACC- | 0.0 |
| | | 62 | |
| Gastric ca.* (liver met) | 36.6 | Melanoma M14 | 26.2 |
| NCI-N87 | | | |
| Bladder | 51.4 | Melanoma LOX | 0.0 |
| | | IMVI | |
| Trachea | 0.0 | Melanoma* (met) | 14.1 |
| | | SK-MEL-5 | |
| Kidney | 56.3 | Adipose | 0.0 |

TABLE BHE
Panel 2D

| | Rel. Exp. (%) | | Rel. Exp. (%) |
|---|---|---|---|
| | Ag1519, | | Ag1519, |
| Tissue Name | Run 145158010 | Tissue Name | Run 145158010 |
| Normal Colon | 81.8 | Kidney Margin | 5.0 |
| | | 8120608 | |
| CC Well to Mod Diff | 6.0 | Kidney Cancer | 0.0 |
| (ODO3866) | | 8120613 | |
| CC Margin (ODO3866) | 7.3 | Kidney Margin | 1.9 |
| | | 8120614 | |
| CC Gr.2 rectosigmoid | 5.8 | Kidney Cancer | 7.6 |
| (ODO3868) | | 9010320 | |
| CC Margin (ODO3868) | 0.0 | Kidney Margin | 5.8 |
| | | 9010321 | |
| CC Mod Diff | 18.6 | Normal Uterus | 4.2 |
| (ODO3920) | | | |
| CC Margin (ODO3920) | 10.6 | Uterus Cancer | 47.3 |
| | | 064011 | |
| CC Gr.2 ascend colon | 8.2 | Normal Thyroid | 21.6 |
| (ODO3921) | | | |
| CC Margin (ODO3921) | 4.8 | Thyroid Cancer | 42.3 |
| | | 064010 | |
| CC from Partial | 47.6 | Thyroid Cancer | 20.9 |
| Hepatectomy | | A302152 | |
| (ODO4309) Mets | | | |
| Liver Margin | 10.4 | Thyroid Margin | 59.5 |
| (ODO4309) | | A302153 | |
| Colon mets to lung | 12.2 | Normal Breast | 71.2 |
| (OD04451-01) | | | |
| Lung Margin (OD04451- | 6.5 | Breast Cancer | 15.7 |
| 02) | | (OD04566) | |
| Normal Prostate 6546-1 | 11.6 | Breast Cancer | 19.9 |
| | | (OD04590-01) | |
| Prostate Cancer | 31.6 | Breast Cancer Mets | 41.5 |
| (OD04410) | | (OD04590-03) | |
| Prostate Margin | 25.5 | Breast Cancer | 33.7 |
| (OD04410) | | Metastasis | |
| | | (OD04655-05) | |
| Prostate Cancer | 27.2 | Breast Cancer | 27.0 |
| (OD04720-01) | | 064006 | |
| Prostate Margin | 31.4 | Breast Cancer 1024 | 48.0 |
| (OD04720-02) | | | |
| Normal Lung 061010 | 25.2 | Breast Cancer | 3.3 |
| | | 9100266 | |
| Lung Met to Muscle | 6.2 | Breast Margin | 7.8 |
| (ODO4286) | | 9100265 | |
| Muscle Margin | 0.0 | Breast Cancer | 24.8 |
| (ODO4286) | | A209073 | |
| Lung Malignant Cancer | 39.0 | Breast Margin | 32.3 |
| (OD03126) | | A209073 | |
| Lung Margin (OD03126) | 12.0 | Normal Liver | 3.5 |
| Lung Cancer (OD04404) | 4.9 | Liver Cancer 064003 | 56.6 |
| Lung Margin (OD04404) | 27.9 | Liver Cancer 1025 | 7.2 |
| Lung Cancer (OD04565) | 11.9 | Liver Cancer 1026 | 1.8 |
| Lung Margin (OD04565) | 1.4 | Liver Cancer 6004-T | 6.0 |
| Lung Cancer (OD04237- | 52.5 | Liver Tissue 6004-N | 0.0 |
| 01) | | | |
| Lung Margin (OD04237- | 20.7 | Liver Cancer 6005-T | 5.6 |
| 02) | | | |
| Ocular Mel Met to Liver | 5.6 | Liver Tissue 6005-N | 0.0 |
| (ODO4310) | | | |
| Liver Margin | 2.2 | Normal Bladder | 24.0 |
| (ODO4310) | | | |
| Melanoma Mets to Lung | 0.0 | Bladder Cancer 1023 | 3.3 |
| (OD04321) | | | |
| Lung Margin (OD04321) | 24.7 | Bladder Cancer | 5.7 |
| | | A302173 | |
| Normal Kidney | 100.0 | Bladder Cancer | 2.9 |
| | | (OD04718-01) | |
| Kidney Ca, Nuclear | 34.4 | Bladder Normal | 0.0 |
| grade 2 (OD04338) | | Adjacent (OD04718- | |
| | | 03) | |
| Kidney Margin | 54.7 | Normal Ovary | 3.9 |
| (OD04338) | | | |
| Kidney Ca Nuclear | 81.8 | Ovarian Cancer | 7.2 |
| grade 1/2 (OD04339) | | 064008 | |
| Kidney Margin | 48.3 | Ovarian Cancer | 38.4 |
| (OD04339) | | (OD04768-07) | |
| Kidney Ca, Clear cell | 11.0 | Ovary Margin | 5.1 |
| type (OD04340) | | (OD04768-08) | |
| Kidney Margin | 56.6 | Normal Stomach | 11.4 |
| (OD04340) | | | |
| Kidney Ca, Nuclear | 3.4 | Gastric Cancer | 6.5 |
| grade 3 (OD04348) | | 9060358 | |
| Kidney Margin | 43.2 | Stomach Margin | 0.0 |
| (OD04348) | | 9060359 | |
| Kidney Cancer | 11.5 | Gastric Cancer | 6.7 |
| (OD04622-01) | | 9060395 | |
| Kidney Margin | 3.5 | Stomach Margin | 4.5 |
| (OD04622-03) | | 9060394 | |
| Kidney Cancer | 17.8 | Gastric Cancer | 0.0 |
| (OD04450-01) | | 9060397 | |
| Kidney Margin | 42.0 | Stomach Margin | 6.7 |
| (OD04450-03) | | 9060396 | |
| Kidney Cancer 8120607 | 0.0 | Gastric Cancer | 16.6 |
| | | 064005 | |

CNS_neurodegeneration_v1.0 Summary: Ag3460 Expression of the CG59540-01 gene is low/undetectable (CT values>35) across the samples in this panel.

General_screening_panel_v1.4 Summary: Ag3460 Expression of the CG59540-01 gene is low/undetectable (CT values>35) across the samples in this panel.

Panel 1.2 Summary: Ag1519 The expression of the CG59540-01 gene appears to be highest in a sample derived from a colon cancer cell line (HCC-2998) (CT=28.2). In addition, there is substantial expression associated with normal kidney and bladder. Thus, the expression of this gene could be used to distinguish these tissues from other tissues in the panel. In addition there was noted expression clustered in ovarian, renal and colon cancer cell lines. Therefore, therapeutic modulation of this gene, through the use of small molecule drugs, antibodies or protein therapeutics might be of use in the treatment of colon cancer, renal cancer or ovarian cancer.

Among tissues with metabolic function, there is moderate expression in fetal and adult heart, adrenal, and pancreas. This expression suggests that therapeutic modulation of the expression or function of the protein encoded by this gene may be useful in the treatment of diseases that involve these tissues, including obesity and diabetes.

In addition, there appears to be higher levels of expression in adult heart (CT=31) when compared to expression in fetal heart (CT=34.4). Thus, expression of this gene could be used to differentiate between adult and fetal heart tissue. Conversely, expression of this gene is higher in fetal lung (CT=34.5) than in adult lung (CT=40). Thus, expression of this gene could also be used to differentiate between adult and fetal lung.

Panel 1.3D Summary: Ag1519 Significant expression the CG59540-01 gene is limited to a sample derived from colorectal tissue (CT=34.3). Thus, expression of this gene could be used to differentiate between this sample and other samples on this panel, and between coloreclal tissue and other normal or malignant tissues.

Panel 2D Summary: Ag1519 The expression of the CG59540-01 gene in panel 2 appears to be highest in a samples derived from normal kidney tissue (CT=32). In addition there appears to be substantial difference in expression between normal kidney adjacent to cancer tissue and the cancer tissue itself. Thus, the expression of this gene could be used to distinguish normal kidney tissue from other samples in the panel. In addition, the expression of this gene could be used to distinguish normal kidney from malignant tissue. Moreover, therapeutic modulation of this gene, through the use of small molecule drugs, antibodies or protein therapeutics might be of use in the treatment of kidney cancer.

Panel 4D Summary: Ag3460 Expression of the CG59540-01 gene is low/undetectable (CT values>35) across the samples in this panel.

BI. CG59280-01 and CG59280-02: Olfactory Receptor

Expression of gene CG59280-01 and CG59280-02 was assessed using the primer-probe set Ag3527, described in Table BIA. Results of the RTQ-PCR runs are shown in Table BIB. Please note that CG59280-02 represents a full-length physical clone of the CG59280-01 gene, validating the prediction of the gene sequence.

Table BIA. Probe Name Ag3527

TABLE BIA
Probe Name Ag3527

| | | | Start | SEQ ID |
|---|---|---|---|---|
| Primers | Sequences | Length | Position | NO: |
| Forward | 5′-atggccattgataggtacgtt-3′ | 21 | 361 | 580 |
| Probe | TET-5′-catctgtaaccctctccgctacccaa-3′-TAMRA | 26 | 384 | 581 |
| Reverse | 5′-ccacagagagctgaacacaga-3′ | 21 | 428 | 582 |

TABLE BIB
Panel 4D

| | Rel. Exp. (%) | | Rel. Exp. (%) |
|---|---|---|---|
| | Ag3527, Run | | Ag3527, Run |
| Tissue Name | 166446354 | Tissue Name | 166446354 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 2.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 4.2 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 4.2 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 4.4 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 5.1 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 4.5 |
| Two Way MLR 7 day | 0.0 | HPAEC TNF alpha + IL- | 0.0 |
| | | 1beta | |
| PBMC rest | 3.1 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 4.5 | Dermal fibroblast IFN | 0.0 |
| | | gamma | |
| Dendritic cells LPS | 5.3 | Dermal fibroblast IL-4 | 0.0 |
| Dendritic cells anti- | 9.9 | IBD Colitis 2 | 10.8 |
| CD40 | | | |
| Monocytes rest | 0.0 | IBD Crohn's | 8.9 |
| Monocytes LPS | 27.0 | Colon | 0.0 |
| Macrophages rest | 9.5 | Lung | 4.6 |
| Macrophages LPS | 4.0 | Thymus | 0.0 |
| HUVEC none | 0.0 | Kidney | 0.0 |
| HUVEC starved | 0.0 | | |

CNS_neurodegeneration_v1.0 Summary: Ag3527 Expression of the CG59280-01 gene is low/undetectable (CT values>35) across the samples in this panel. (Data not shown.)

General_screening_panel_v1.4 Summary: Ag3527 Expression of the CG59280-01 gene is low/undetectable (CT values>35) across the samples in this panel. (Data not shown.) This gene encodes a G protein-coupled receptor (GPCR), a type of cell surface receptor involved in signal transduction. It is most similar to members of the odorant receptor subfamily of GPCRs. Based on analogy to other odorant receptor genes, we predict that expression of this gene may be highest in nasal epithelium, a sample not represented on this panel.

Panel 4D Summary: Ag3527 Highest expression of the CG59280-01 gene is seen in the liver cirrhosis sample (CT=31.81). Thus, expression of this gene could be used to differentiate between this sample from the other samples on this panel and as a marker to detect the presence of liver cirrhosis. Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

BJ. CG59568-01: GPCR

Expression of gene CG59568-01 was assessed using the primer-probe set Ag3474, described in Table BJA. Results of the RTQ-PCR runs are shown in Table BJB.

Table BJA. Probe Name Ag3474

TABLE BJA
Probe Name Ag3474

| | | | Start | SEQ ID |
|---|---|---|---|---|
| Primers | Sequences | Length | Position | NO: |
| Forward | 5′-ttacagcaatcaccatggtctt-3′ | 22 | 475 | 760 |
| Probe | TET-5′-accttctgtggaccctatgaaactg-3′-TAMRA | 26 | 510 | 761 |
| Reverse | 5′-gggtgaagtcacaaaagaagtg-3′ | 22 | 537 | 762 |

TABLE BJB
Panel 4D

| | Rel. Exp. (%) | | Rel. Exp. (%) |
|---|---|---|---|
| | Ag3474, Run | | Ag3474, Run |
| Tissue Name | 166417193 | Tissue Name | 166417193 |
| Secondary Th1 act | 7.1 | HUVEC IL-1beta | 0.0 |
| Secondary Th2 act | 0.0 | HUVEC IFN gamma | 0.0 |
| Secondary Tr1 act | 4.2 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 2.2 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 3.3 | Bronchial epithelium | 0.0 |
| | | TNF alpha + IL1beta | |
| Primary Th2 rest | 5.9 | Small airway epithelium | 0.0 |
| | | none | |
| Primary Tr1 rest | 0.0 | Small airway epithelium | 0.3 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 1.7 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 3.1 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.5 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 3.6 | Lupus kidney | 0.0 |
| LAK cells IL-2 + IFN | 3.9 | NCI-H292 none | 0.0 |
| gamma | | | |
| LAK cells IL-2 + IL-18 | 2.4 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 6.2 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.3 |
| ionomycin | | | |
| B lymphocytes PWM | 8.4 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 1.6 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 1.5 | Dermal fibroblast IL-4 | 0.0 |
| Dendritic cells anti- | 0.0 | IBD Colitis 2 | 11.9 |
| CD40 | | | |
| Monocytes rest | 0.0 | IBD Crohn's | 0.0 |
| Monocytes LPS | 5.8 | Colon | 1.5 |
| Macrophages rest | 3.4 | Lung | 0.0 |
| Macrophages LPS | 0.0 | Thymus | 3.8 |
| HUVEC none | 0.0 | Kidney | 6.7 |
| HUVEC starved | 0.0 | | |

CNS_neurodegeneration_v1.0 Summary: Ag3474 Expression of the CG59568-01 gene is low/undetectable (CT values>35) across the samples in this panel. (Data not shown.)

General_screening_panel_v1.4 Summary: Ag3474 Expression of the CG59568-01 gene is low/undetectable (CT values>35) across the samples in this panel. (Data not shown.) This gene encodes a G protein-coupled receptor (GPCR), a type of cell surface receptor involved in signal transduction. It is most similar to members of the odorant receptor subfamily of GPCRs. Based on analogy to other odorant receptor genes, we predict that expression of this gene may be highest in nasal epithelium, a sample not represented on this panel.

Panel 4D Summary: Ag3474 Highest expression of the CG59280-01 gene is seen in the liver cirrhosis sample (CT=31.37). Thus, expression of this gene could be used to differentiate between this sample from the other samples on this panel and as a marker to detect the presence of liver cirrhosis. Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

### REFERENCES

1. Mark M. D., Wittemann S., Herlitze S. (2000) G protein modulation of recombinant P/Q-type calcium channels by regulators of G protein signalling proteins. J. Physiol 528 Pt 1: 65-77.

BK. CG59224-01 and CG59216-01: GPCR

Expression of gene CG59224-01 and variant CG59216-01 was assessed using the primer-probe sets Ag3400 and Ag3405, described in Tables BKA and BKB. Results of the RTQ-PCR runs are shown in Table BKC.

Table BKA. Probe Name Ag3400

TABLE BKA
Probe Name Ag3400

| | | | Start | SEQ ID |
|---|---|---|---|---|
| Primers | Sequences | Length | Position | NO: |
| Forward | 5′-tctctgacctagggctgtctct-3′ | 22 | 225 | 584 |
| Probe | TET-5′-tcttccttacccatcactttgggact-3′-TAMRA | 26 | 248 | 585 |
| Reverse | 5′-catgaatttcatggacatcaaa-3′ | 22 | 281 | 586 |

Table BKB. Probe Name Ag3405

TABLE BKB
Probe Name Ag3405

| | | | Start | SEQ ID |
|---|---|---|---|---|
| Primers | Sequences | Length | Position | NO: |
| Forward | 5′-cacatctgtgctgtgcttatct-3′ | 22 | 746 | 587 |
| Probe | TET-5′-agtgctgccatgctccaccagttt-3′-TAMRA | 24 | 785 | 588 |
| Reverse | 5′-acgtggatcataggagacacat-3′ | 22 | 816 | 589 |

TABLE BKC
General_screening_panel_v1.4

| | Rel. Exp. (%) | Rel. Exp. (%) | | Rel. Exp. (%) | Rel. Exp. (%) |
|---|---|---|---|---|---|
| | Ag3400, Run | Ag3405, Run | | Ag3400, Run | Ag3405, Run |
| Tissue Name | 216822602 | 216838741 | Tissue Name | 216822602 | 216838741 |
| Adipose | 0.0 | 0.0 | Renal ca. TK-10 | 0.0 | 0.0 |
| Melanoma* | 0.0 | 0.0 | Bladder | 1.2 | 0.0 |
| Hs688(A).T | | | | | |
| Melanoma* | 0.0 | 0.0 | Gastric ca. (liver | 0.0 | 0.0 |
| Hs688(B).T | | | met.) NCI-N87 | | |
| Melanoma* | 0.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 0.0 | Colon ca. | 0.0 | 0.0 |
| SK-MEL-5 | | | SW480 | | |
| Squamous | 0.0 | 0.0 | Colon ca.* | 0.0 | 0.0 |
| cell | | | (SW480 met) | | |
| carcinoma | | | SW620 | | |
| SCC-4 | | | | | |
| Testis Pool | 0.0 | 0.0 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 3.2 | 1.8 | Colon ca. CaCo-2 | 0.0 | 0.0 |
| Placenta | 0.0 | 0.0 | Colon cancer | 0.0 | 0.0 |
| | | | tissue | | |
| Uterus Pool | 0.0 | 0.0 | Colon ca. | 0.0 | 0.0 |
| | | | SW1116 | | |
| Ovarian ca. | 0.0 | 0.0 | Colon ca. Colo- | 0.0 | 0.0 |
| OVCAR-3 | | | 205 | | |
| Ovarian ca. | 1.0 | 0.0 | Colon ca. SW-48 | 0.0 | 0.0 |
| SK-OV-3 | | | | | |
| Ovarian ca. | 0.0 | 0.0 | Colon Pool | 0.0 | 0.0 |
| OVCAR-4 | | | | | |
| Ovarian ca. | 0.0 | 0.0 | Small Intestine | 0.0 | 0.0 |
| OVCAR-5 | | | Pool | | |
| Ovarian ca. | 0.0 | 0.0 | Stomach Pool | 0.0 | 0.0 |
| IGROV-1 | | | | | |
| Ovarian ca. | 0.0 | 0.0 | Bone Marrow | 0.0 | 0.0 |
| OVCAR-8 | | | Pool | | |
| Ovary | 0.0 | 0.0 | Fetal Heart | 0.0 | 0.0 |
| Breast ca. | 0.0 | 0.0 | Heart Pool | 1.7 | 1.3 |
| MCF-7 | | | | | |
| Breast ca. | 0.0 | 0.0 | Lymph Node | 0.0 | 0.0 |
| MDA-MB- | | | Pool | | |
| 231 | | | | | |
| Breast ca. BT | 0.0 | 0.0 | Fetal Skeletal | 0.0 | 0.0 |
| 549 | | | Muscle | | |
| Breast ca. | 0.0 | 0.0 | Skeletal Muscle | 0.0 | 0.0 |
| T47D | | | Pool | | |
| Breast ca. | 0.0 | 0.5 | Spleen Pool | 0.0 | 0.0 |
| MDA-N | | | | | |
| Breast Pool | 0.0 | 0.5 | Thymus Pool | 0.0 | 0.5 |
| Trachea | 0.0 | 0.0 | CNS cancer | 0.0 | 0.0 |
| | | | (glio/astro) U87- | | |
| | | | MG | | |
| Lung | 0.0 | 0.0 | CNS cancer | 0.0 | 0.0 |
| | | | (glio/astro) U- | | |
| | | | 118-MG | | |
| Fetal Lung | 0.0 | 0.0 | CNS cancer | 0.0 | 0.0 |
| | | | (neuro; met) SK- | | |
| | | | N-AS | | |
| Lung ca. | 0.0 | 0.0 | CNS cancer | 0.0 | 0.0 |
| NCI-N417 | | | (astro) SF-539 | | |
| Lung ca. LX-1 | 0.0 | 0.0 | CNS cancer | 0.0 | 0.0 |
| | | | (astro) SNB-75 | | |
| Lung ca. | 2.5 | 3.3 | CNS cancer | 0.0 | 0.0 |
| NCI-H146 | | | (glio) SNB-19 | | |
| Lung ca. | 100.0 | 100.0 | CNS cancer | 0.0 | 0.0 |
| SHP-77 | | | (glio) SF-295 | | |
| Lung ca. | 0.0 | 0.0 | Brain | 0.0 | 0.0 |
| A549 | | | (Amygdala) Pool | | |
| Lung ca. | 0.0 | 0.0 | Brain | 0.0 | 0.0 |
| NCI-H526 | | | (cerebellum) | | |
| Lung ca. | 0.0 | 0.0 | Brain (fetal) | 0.0 | 0.0 |
| NCI-H23 | | | | | |
| Lung ca. | 0.0 | 0.0 | Brain | 0.0 | 0.0 |
| NCI-H460 | | | (Hippocampus) | | |
| | | | Pool | | |
| Lung ca. | 0.0 | 0.0 | Cerebral Cortex | 0.0 | 0.6 |
| HOP-62 | | | | | |
| Lung ca. | 0.0 | 0.0 | Brain (Substantia | 0.0 | 0.0 |
| NCI-H522 | | | nigra) Pool | | |
| Liver | 0.0 | 0.0 | Brain | 0.0 | 0.0 |
| | | | (Thalamus) Pool | | |
| Fetal Liver | 0.0 | 0.0 | Brain (whole) | 0.0 | 0.0 |
| Liver ca. | 0.0 | 0.0 | Spinal Cord Pool | 0.0 | 0.0 |
| HepG2 | | | | | |
| Kidney Pool | 0.0 | 0.4 | Adrenal Gland | 0.0 | 0.0 |
| Fetal Kidney | 10.8 | 2.6 | Pituitary gland | 0.0 | 0.0 |
| | | | Pool | | |
| Renal ca. | 0.0 | 0.0 | Salivary Gland | 0.0 | 0.0 |
| 786-0 | | | | | |
| Renal ca. | 0.0 | 0.0 | Thyroid (female) | 0.0 | 0.0 |
| A498 | | | | | |
| Renal ca. | 0.0 | 0.0 | Pancreatic ca. | 0.0 | 0.0 |
| ACHN | | | CAPAN2 | | |
| Renal ca. | 0.0 | 0.0 | Pancreas Pool | 0.0 | 1.2 |
| UO-31 | | | | | |

CNS_neurodegeneration_v1.0 Summary: Ag3400/Ag3405 Expression of the CG59224-01 gene is low/undetectable (CT values>35) across the samples in this panel. (Data not shown.)

General_screening_panel_v1.4 Summary: Ag3400/Ag3405 Two experiments with two different probe and primer sets produce results that are in excellent agreement, with significant expression of the CG59224-01 gene exclusively in a lung cancer cell line sample (CTs=30-33). Therefore, expression of this gene may be used to distinguish this sample from other samples on this panel and as a marker for lung cancer. Furthermore, therapeutic modulation of the activity of the GPCR encoded by this gene may be beneficial in the treatment of lung cancer.

Panel 4D Summary: Ag3400/Ag3405 Expression of the CG59224-01 gene is low/undetectable (CT values>35) across the samples in this panel. (Data not shown.) This gene encodes a G protein-coupled receptor (GPCR), a type of cell surface receptor involved in signal transduction. It is most similar to members of the odorant receptor subfamily of GPCRs. Based on analogy to other odorant receptor genes, we predict that expression of this gene may be highest in nasal epithelium, a sample not represented on this panel.

BL. CG59214-01 and CG59214-01: GPCR

Expression of gene CG59214-01 and CG59214-01 was assessed using the primer-probe sets Ag3398 and Ag3404, described in Tables BLA and BLB. Results of the RTQ-PCR runs are shown in Tables BLC and BLD.

Table BLA. Probe Name Ag3398

TABLE BLA
Probe Name Ag3398

| | | | Start | SEQ |
|---|---|---|---|---|
| Primers | Sequences | Length | Position | ID NO: |
| Forward | 5′-atacttgcatctcccacatctg-3′ | 22 | 724 | 590 |
| Probe | TET-5′-caccaatgattgggctatctatgatcca-3′-TAMRA | 28 | 766 | 591 |
| Reverse | 5′-tgaggaagcattctgtccatag-3′ | 22 | 797 | 592 |

Table BLB. Probe Name Ag3404

TABLE BLB
Probe Name Ag3404

| | | | Start | SEQ ID |
|---|---|---|---|---|
| Primers | Sequences | Length | Position | NO: |
| Forward | 5′-atacttgcatctcccacatctg-3′ | 22 | 724 | 593 |
| Probe | TET-5′-caccaatgattgggctatctatgatcca-3′-TAMRA | 28 | 766 | 594 |
| Reverse | 5′-tgaggaagcattctgtccatag-3′ | 22 | 797 | 595 |

TABLE BLC
General_screening_panel_v1.4

| | Rel. Exp. (%) | Rel. Exp. (%) | | Rel. Exp. (%) | Rel. Exp. (%) |
|---|---|---|---|---|---|
| | Ag3398, Run | Ag3404, Run | | Ag3398, Run | Ag3404, Run |
| Tissue Name | 216822567 | 216838380 | Tissue Name | 216822567 | 216838380 |
| Adipose | 0.0 | 0.0 | Renal ca. TK-10 | 0.0 | 0.0 |
| Melanoma* | 0.0 | 0.0 | Bladder | 0.0 | 0.0 |
| Hs688(A).T | | | | | |
| Melanoma* | 0.0 | 0.0 | Gastric ca. (liver | 0.0 | 0.0 |
| Hs688(B).T | | | met.) NCI-N87 | | |
| Melanoma* | 0.0 | 0.0 | Gastric ca. | 0.0 | 0.0 |
| M14 | | | KATO III | | |
| Melanoma* | 0.0 | 0.0 | Colon ca. SW- | 0.0 | 0.0 |
| LOXIMVI | | | 948 | | |
| Melanoma* | 0.0 | 88.9 | Colon ca. | 0.0 | 0.0 |
| SK-MEL-5 | | | SW480 | | |
| Squamous | 0.0 | 0.0 | Colon ca.* | 0.0 | 0.0 |
| cell | | | (SW480 met) | | |
| carcinoma | | | SW620 | | |
| SCC-4 | | | | | |
| Testis Pool | 0.0 | 0.0 | Colon ca. HT29 | 0.0 | 0.0 |
| Prostate ca.* | 0.0 | 0.0 | Colon ca. HCT- | 0.0 | 0.0 |
| (bone met) | | | 116 | | |
| PC-3 | | | | | |
| Prostate Pool | 0.0 | 0.0 | Colon ca. CaCo-2 | 0.0 | 0.0 |
| Placenta | 0.0 | 0.0 | Colon cancer | 0.0 | 0.0 |
| | | | tissue | | |
| Uterus Pool | 0.0 | 0.0 | Colon ca. | 0.0 | 0.0 |
| | | | SW1116 | | |
| Ovarian ca. | 0.0 | 0.0 | Colon ca. Colo- | 0.0 | 0.0 |
| OVCAR-3 | | | 205 | | |
| Ovarian ca. | 0.0 | 0.0 | Colon ca. SW-48 | 0.0 | 0.0 |
| SK-OV-3 | | | | | |
| Ovarian ca. | 0.0 | 0.0 | Colon Pool | 0.0 | 0.0 |
| OVCAR-4 | | | | | |
| Ovarian ca. | 0.0 | 0.0 | Small Intestine | 0.0 | 8.9 |
| OVCAR-5 | | | Pool | | |
| Ovarian ca. | 0.0 | 0.0 | Stomach Pool | 0.0 | 15.6 |
| IGROV-1 | | | | | |
| Ovarian ca. | 0.0 | 0.0 | Bone Marrow | 0.0 | 0.0 |
| OVCAR-8 | | | Pool | | |
| Ovary | 0.0 | 7.5 | Fetal Heart | 0.0 | 0.0 |
| Breast ca. | 0.0 | 0.0 | Heart Pool | 0.0 | 0.0 |
| MCF-7 | | | | | |
| Breast ca. | 0.0 | 0.0 | Lymph Node | 0.0 | 0.0 |
| MDA-MB- | | | Pool | | |
| 231 | | | | | |
| Breast ca. BT | 0.0 | 0.0 | Fetal Skeletal | 0.0 | 0.0 |
| 549 | | | Muscle | | |
| Breast ca. | 0.0 | 0.0 | Skeletal Muscle | 0.0 | 0.0 |
| T47D | | | Pool | | |
| Breast ca. | 6.9 | 0.0 | Spleen Pool | 0.0 | 0.0 |
| MDA-N | | | | | |
| Breast Pool | 0.0 | 0.0 | Thymus Pool | 0.0 | 0.0 |
| Trachea | 0.0 | 0.0 | CNS cancer | 0.0 | 0.0 |
| | | | (glio/astro) U87- | | |
| | | | MG | | |
| Lung | 0.0 | 0.0 | CNS cancer | 0.0 | 12.5 |
| | | | (glio/astro) U- | | |
| | | | 118-MG | | |
| Fetal Lung | 0.0 | 0.0 | CNS cancer | 0.0 | 0.0 |
| | | | (neuro; met) SK- | | |
| | | | N-AS | | |
| Lung ca. | 0.0 | 0.0 | CNS cancer | 0.0 | 0.0 |
| NCI-N417 | | | (astro) SF-539 | | |
| Lung ca. LX-1 | 0.0 | 0.0 | CNS cancer | 0.0 | 0.0 |
| | | | (astro) SNB-75 | | |
| Lung ca. | 0.0 | 0.0 | CNS cancer | 0.0 | 0.0 |
| NCI-H146 | | | (glio) SNB-19 | | |
| Lung ca. | 100.0 | 100.0 | CNS cancer | 0.0 | 0.0 |
| SHP-77 | | | (glio) SF-295 | | |
| Lung ca. | 0.0 | 0.0 | Brain | 0.0 | 0.0 |
| A549 | | | (Amygdala) Pool | | |
| Lung ca. | 0.0 | 0.0 | Brain | 0.0 | 0.0 |
| NCI-H526 | | | (cerebellum) | | |
| Lung ca. | 0.0 | 0.0 | Brain (fetal) | 0.0 | 0.0 |
| NCI-H23 | | | | | |
| Lung ca. | 0.0 | 0.0 | Brain | 0.0 | 0.0 |
| NCI-H460 | | | (Hippocampus) | | |
| | | | Pool | | |
| Lung ca. | 0.0 | 0.0 | Cerebral Cortex | 0.0 | 0.0 |
| HOP-62 | | | Pool | | |
| Lung ca. | 0.0 | 0.0 | Brain (Substantia | 0.0 | 0.0 |
| NCI-H522 | | | nigra) Pool | | |
| Liver | 0.0 | 0.0 | Brain | 4.3 | 0.0 |
| | | | (Thalamus) Pool | | |
| Fetal Liver | 0.0 | 0.0 | Brain (whole) | 0.0 | 0.0 |
| Liver ca. | 0.0 | 0.0 | Spinal Cord Pool | 0.0 | 0.0 |
| HepG2 | | | | | |
| Kidney Pool | 0.0 | 0.0 | Adrenal Gland | 0.0 | 0.0 |
| Fetal Kidney | 11.1 | 8.5 | Pituitary gland | 0.0 | 0.0 |
| | | | Pool | | |
| Renal ca. | 0.0 | 0.0 | Salivary Gland | 0.0 | 0.0 |
| 786-0 | | | | | |
| Renal ca. | 0.0 | 0.0 | Thyroid (female) | 0.0 | 0.0 |
| A498 | | | | | |
| Renal ca. | 0.0 | 0.0 | Pancreatic ca. | 0.0 | 0.0 |
| ACHN | | | CAPAN2 | | |
| Renal ca. | 0.0 | 0.0 | Pancreas Pool | 0.0 | 0.0 |
| UO-31 | | | | | |

TABLE BLD
Panel 4D

| | Rel. Exp. (%) | | Rel. Exp. (%) |
|---|---|---|---|
| | Ag3404, Run | | Ag3404, Run |
| Tissue Name | 165825947 | Tissue Name | 165825947 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 5.8 |
| | | PMA/ionomycin | |
| 2ry Th1/Th2/Tr1_anti- | 8.9 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 3.9 |
| Dendritic cells anti- | 0.0 | IBD Colitis 2 | 7.2 |
| CD40 | | | |
| Monocytes rest | 0.0 | IBD Crohn's | 4.0 |
| Monocytes LPS | 0.0 | Colon | 5.5 |
| Macrophages rest | 0.0 | Lung | 3.1 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag3398/Ag3404 Expression of the CG59222-01 gene is low/undetectable (CT values>35) across the samples in this panel. (Data not shown.)

General_screening_panel_v1.4 Summary: Ag3398/Ag3404 Two experiments with two different probe and primer sets produce results that are in excellent agreement, with significant expression of the CG59222-01 gene exclusively in a lung cancer cell line sample (CT=33.8). Therefore, expression of this gene may be used to this sample from other samples on this panel and as a marker for lung cancer. Furthermore, therapeutic modulation of the activity of the GPCR encoded by this gene may be beneficial in the treatment of lung cancer.

Panel 4D Summary: Ag3404 Highest expression of the CG59222-01 gene is seen in the liver cirrhosis sample (CT=32.65). Thus, expression of this gene could be used to differentiate between this sample from the other samples on this panel and as a marker to detect the presence of liver cirrhosis. Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis. Ag3398 Expression of CG59222-01 gene is low/undetectable (CTs>35) across all of the samples on this panel (Data not shown).

BM. CG159220-01: GPCR

Expression of gene CG59220-01 was assessed using the primer-probe set Ag3402, described in Table BMA. Results of the RTQ-PCR runs are shown in Tables BMB, BMC and BMD.

Table BMA. Probe Name Ag3402

TABLE BMA
Probe Name Ag3402

| | | | Start | SEQ ID |
|---|---|---|---|---|
| Primers | Sequences | Length | Position | NO: |
| Forward | 5′-ctccacacacccatgtacttct-3′ | 22 | 160 | 596 |
| Probe | TET-5′-cttggatctctgccattcctctgtca-3′-TAMRA | 26 | 201 | 597 |
| Reverse | 5′-aggaggttctccaacagcttag-3′ | 22 | 233 | 598 |

TABLE BMB
CNS_neurodegeneration_v1.0

| | Rel. | | Rel. |
|---|---|---|---|
| | Exp. (%) | | Exp. (%) |
| | Ag3402, | | Ag3402, |
| | Run | | Run |
| Tissue Name | 210349784 | Tissue Name | 210349784 |
| AD 1 Hippo | 9.0 | Control (Path) 3 | 18.6 |
| | | Temporal Ctx | |
| AD 2 Hippo | 46.3 | Control (Path) 4 | 56.6 |
| | | Temporal Ctx | |
| AD 3 Hippo | 8.8 | AD 1 Occipital Ctx | 21.6 |
| AD 4 Hippo | 24.1 | AD 2 Occipital Ctx | 0.0 |
| | | (Missing) | |
| AD 5 Hippo | 52.9 | AD 3 Occipital Ctx | 15.0 |
| AD 6 Hippo | 39.2 | AD 4 Occipital Ctx | 48.0 |
| Control 2 Hippo | 41.8 | AD 5 Occipital Ctx | 56.3 |
| Control 4 Hippo | 18.2 | AD 6 Occipital Ctx | 31.4 |
| Control (Path) 3 | 13.7 | Control 1 Occipital | 14.4 |
| Hippo | | Ctx | |
| AD 1 Temporal | 27.4 | Control 2 Occipital | 55.5 |
| Ctx | | Ctx | |
| AD 2 Temporal | 57.0 | Control 3 Occipital | 42.3 |
| Ctx | | Ctx | |
| AD 3 Temporal | 10.2 | Control 4 Occipital | 21.2 |
| Ctx | | Ctx | |
| AD 4 Temporal | 54.3 | Control (Path) 1 | 100.0 |
| Ctx | | Occipital Ctx | |
| AD 5 Inf Temporal | 42.9 | Control (Path) 2 | 27.7 |
| Ctx | | Occipital Ctx | |
| AD 5 Sup | 28.7 | Control (Path) 3 | 9.0 |
| Temporal Ctx | | Occipital Ctx | |
| AD 6 Inf Temporal | 41.8 | Control (Path) 4 | 34.2 |
| Ctx | | Occipital Ctx | |
| AD 6 Sup | 51.4 | Control 1 Parietal | 23.5 |
| Temporal Ctx | | Ctx | |
| Control 1 | 18.2 | Control 2 Parietal | 28.5 |
| Temporal Ctx | | Ctx | |
| Control 2 | 43.2 | Control 3 Parietal | 29.5 |
| Temporal Ctx | | Ctx | |
| Control 3 | 32.8 | Control (Path) 1 | 99.3 |
| Temporal Ctx | | Parietal Ctx | |
| Control 3 | 20.0 | Control (Path) 2 | 46.7 |
| Temporal Ctx | | Parietal Ctx | |
| Control (Path) 1 | 87.1 | Control (Path) 3 | 12.0 |
| Temporal Ctx | | Parietal Ctx | |
| Control (Path) 2 | 60.3 | Control (Path) 4 | 82.4 |
| Temporal Ctx | | Parietal Ctx | |

TABLE BMC
General_screening_panel_v1.4

| | Rel. | | Rel. |
|---|---|---|---|
| | Exp. (%) | | Exp. (%) |
| | Ag3402, | | Ag3402, |
| | Run | | Run |
| Tissue Name | 216823314 | Tissue Name | 216823314 |
| Adipose | 14.7 | Renal ca. TK-10 | 2.5 |
| Melanoma* | 10.1 | Bladder | 28.7 |
| Hs688(A).T | | | |
| Melanoma* | 1.4 | Gastric ca. (liver met.) | 17.9 |
| Hs688(B).T | | NCI-N87 | |
| Melanoma* M14 | 0.9 | Gastric ca. KATO III | 0.7 |
| Melanoma* | 0.8 | Colon ca. SW-948 | 0.0 |
| LOXIMVI | | | |
| Melanoma* SK- | 2.1 | Colon ca. SW480 | 1.7 |
| MEL-5 | | | |
| Squamous cell | 2.9 | Colon ca.* (SW480 | 1.8 |
| carcinoma SCC-4 | | met) SW620 | |
| Testis Pool | 25.9 | Colon ca. HT29 | 1.3 |
| Prostate ca.* (bone | 1.2 | Colon ca. HCT-116 | 0.0 |
| met) PC-3 | | | |
| Prostate Pool | 16.0 | Colon ca. CaCo-2 | 0.7 |
| Placenta | 1.3 | Colon cancer tissue | 7.4 |
| Uterus Pool | 15.6 | Colon ca. SW1116 | 0.0 |
| Ovarian ca. | 3.1 | Colon ca. Colo-205 | 0.0 |
| OVCAR-3 | | | |
| Ovarian ca. SK- | 6.0 | Colon ca. SW-48 | 0.0 |
| OV-3 | | | |
| Ovarian ca. | 0.6 | Colon Pool | 49.7 |
| OVCAR-4 | | | |
| Ovarian ca. | 2.9 | Small Intestine Pool | 53.6 |
| OVCAR-5 | | | |
| Ovarian ca. | 1.6 | Stomach Pool | 15.8 |
| IGROV-1 | | | |
| Ovarian ca. | 1.6 | Bone Marrow Pool | 14.5 |
| OVCAR-8 | | | |
| Ovary | 48.3 | Fetal Heart | 9.1 |
| Breast ca. MCF-7 | 0.6 | Heart Pool | 39.5 |
| Breast ca. MDA- | 0.0 | Lymph Node Pool | 39.2 |
| MB-231 | | | |
| Breast ca. BT 549 | 2.4 | Fetal Skeletal Muscle | 5.8 |
| Breast ca. T47D | 1.3 | Skeletal Muscle Pool | 47.6 |
| Breast ca. MDA-N | 0.4 | Spleen Pool | 22.2 |
| Breast Pool | 31.6 | Thymus Pool | 12.9 |
| Trachea | 5.9 | CNS cancer | 0.8 |
| | | (glio/astro) U87-MG | |
| Lung | 16.6 | CNS cancer | 3.4 |
| | | (glio/astro) U-118-MG | |
| Fetal Lung | 30.8 | CNS cancer | 1.5 |
| | | (neuro; met) SK-N-AS | |
| Lung ca. NCI-N417 | 0.0 | CNS cancer (astro) | 0.0 |
| | | SF-539 | |
| Lung ca. LX-1 | 9.7 | CNS cancer (astro) | 1.1 |
| | | SNB-75 | |
| Lung ca. NCI-H146 | 0.0 | CNS cancer (glio) | 1.1 |
| | | SNB-19 | |
| Lung ca. SHP-77 | 0.6 | CNS cancer (glio) SF- | 4.1 |
| | | 295 | |
| Lung ca. A549 | 0.6 | Brain (Amygdala) | 28.1 |
| | | Pool | |
| Lung ca. NCI-H526 | 0.0 | Brain (cerebellum) | 35.1 |
| Lung ca. NCI-H23 | 5.9 | Brain (fetal) | 12.3 |
| Lung ca. NCI-H460 | 0.9 | Brain (Hippocampus) | 39.8 |
| | | Pool | |
| Lung ca. HOP-62 | 1.6 | Cerebral Cortex Pool | 88.9 |
| Lung ca. NCI-H522 | 7.1 | Brain (Substantia | 43.2 |
| | | nigra) Pool | |
| Liver | 0.0 | Brain (Thalamus) Pool | 74.2 |
| Fetal Liver | 2.5 | Brain (whole) | 26.1 |
| Liver ca. HepG2 | 0.7 | Spinal Cord Pool | 100.0 |
| Kidney Pool | 51.8 | Adrenal Gland | 24.1 |
| Fetal Kidney | 16.7 | Pituitary gland Pool | 8.6 |
| Renal ca. 786-0 | 0.8 | Salivary Gland | 3.2 |
| Renal ca. A498 | 0.0 | Thyroid (female) | 8.9 |
| Renal ca. ACHN | 1.4 | Pancreatic ca. | 0.8 |
| | | CAPAN2 | |
| Renal ca. UO-31 | 1.5 | Pancreas Pool | 24.8 |

TABLE BMD
Panel 4D

| | Rel. Exp. (%) | | Rel. Exp. (%) |
|---|---|---|---|
| | Ag3402, Run | | Ag3402, Run |
| Tissue Name | 165825209 | Tissue Name | 165825209 |
| Secondary Th1 act | 0.0 | HUVEC IL-1beta | 3.5 |
| Secondary Th2 act | 0.0 | HUVEC IFN gamma | 6.5 |
| Secondary Tr1 act | 3.3 | HUVEC TNF alpha + | 15.4 |
| | | IFN gamma | |
| Secondary Th1 rest | 0.0 | HUVEC TNF alpha + | 2.9 |
| | | IL4 | |
| Secondary Th2 rest | 5.2 | HUVEC IL-11 | 9.0 |
| Secondary Tr1 rest | 0.0 | Lung Microvascular EC | 2.7 |
| | | none | |
| Primary Th1 act | 5.4 | Lung Microvascular EC | 0.0 |
| | | TNF alpha + IL-1beta | |
| Primary Th2 act | 0.0 | Microvascular Dermal | 3.0 |
| | | EC none | |
| Primary Tr1 act | 0.0 | Microsvasular Dermal | 10.7 |
| | | EC TNF alpha + IL-1beta | |
| Primary Th1 rest | 3.3 | Bronchial epithelium | 18.0 |
| | | TNF alpha + IL1beta | |
| Primary Th2 rest | 5.1 | Small airway epithelium | 0.0 |
| | | none | |
| Primary Tr1 rest | 0.0 | Small airway epithelium | 79.0 |
| | | TNF alpha + IL-1beta | |
| CD45RA CD4 | 2.7 | Coronery artery SMC rest | 0.0 |
| lymphocyte act | | | |
| CD45RO CD4 | 0.0 | Coronery artery SMC | 2.5 |
| lymphocyte act | | TNF alpha + IL-1beta | |
| CD8 lymphocyte act | 0.0 | Astrocytes rest | 11.3 |
| Secondary CD8 | 3.0 | Astrocytes TNF alpha + | 75.3 |
| lymphocyte rest | | IL-1beta | |
| Secondary CD8 | 0.0 | KU-812 (Basophil) rest | 2.6 |
| lymphocyte act | | | |
| CD4 lymphocyte none | 0.0 | KU-812 (Basophil) | 6.8 |
| | | PMA/ionomycin | |
| 2ry Th1/Th2/Tr1_anti- | 1.6 | CCD1106 | 0.0 |
| CD95 CH11 | | (Keratinocytes) none | |
| LAK cells rest | 0.0 | CCD1106 | 27.9 |
| | | (Keratinocytes) | |
| | | TNF alpha + IL-1beta | |
| LAK cells IL-2 | 5.1 | Liver cirrhosis | 90.8 |
| LAK cells IL-2 + IL-12 | 3.0 | Lupus kidney | 51.8 |
| LAK cells IL-2 + IFN | 4.5 | NCI-H292 none | 28.1 |
| gamma | | | |
| LAK cells IL-2 + IL-18 | 23.2 | NCI-H292 IL-4 | 10.6 |
| LAK cells | 0.0 | NCI-H292 IL-9 | 23.5 |
| PMA/ionomycin | | | |
| NK Cells IL-2 rest | 6.2 | NCI-H292 IL-13 | 4.8 |
| Two Way MLR 3 day | 12.1 | NCI-H292 IFN gamma | 10.3 |
| Two Way MLR 5 day | 3.1 | HPAEC none | 5.8 |
| Two Way MLR 7 day | 5.8 | HPAEC TNF alpha + IL- | 0.0 |
| | | 1beta | |
| PBMC rest | 0.0 | Lung fibroblast none | 3.7 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 6.4 |
| Ramos (B cell) | 0.0 | Lung fibroblast IL-13 | 3.2 |
| ionomycin | | | |
| B lymphocytes PWM | 2.6 | Lung fibroblast IFN | 10.5 |
| | | gamma | |
| B lymphocytes CD40L | 1.8 | Dermal fibroblast | 0.0 |
| and IL-4 | | CCD1070 rest | |
| EOL-1 dbcAMP | 0.0 | Dermal fibroblast | 13.7 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 6.5 | Dermal fibroblast IFN | 0.0 |
| | | gamma | |
| Dendritic cells LPS | 4.7 | Dermal fibroblast IL-4 | 6.3 |
| Dendritic cells anti- | 0.0 | IBD Colitis 2 | 27.0 |
| CD40 | | | |
| Monocytes rest | 24.8 | IBD Crohn's | 9.3 |
| Monocytes LPS | 3.1 | Colon | 100.0 |
| Macrophages rest | 4.0 | Lung | 24.7 |
| Macrophages LPS | 0.0 | Thymus | 59.5 |
| HUVEC none | 6.5 | Kidney | 27.0 |
| HUVEC starved | 41.2 | | |

CNS_neurodegeneration_v1.0 Summary: Ag3402 The CG59220-01 gene is expressed at low levels throughout the CNS, including in amygdala, substantia nigra, thalamus, cerebellum, cerebral cortex, and spinal cord. The GPCR family of receptors contains a large number of neurotransmitter receptors, including the dopamine, serotonin, a and b-adrenerlyic, acetylcholine muscarinic, histamine, peptide, and metabotropic glutamate receptors. GPCRs are excellent drug targets in various neurologic and psychiatric diseases. All antipsychotics have been shown to act at the dopamine D2 receptor; similarly novel antipsychotics also act at the serotonergic receptor, and often the muscarinic and adrenergic receptors as well. While the majority of antidepressants can be classified as selective serotonin reuptake inhibitors, blockade of the 5-HT1A and a2 adrenergic receptors increases the effects of these drugs. The GPCRs are also of use as drug targets in the treatment of stroke. Blockade of the glutamate receptors may decrease the neuronal death resulting from excitotoxicity; further more the purinergic receptors have also been implicated as drug targets in the treatment of cerebral ischemia. The b-adrenergic receptors have been implicated in the treatment of ADHD with Ritalin, while the a-adrenergic receptors have been implicated in memory. Therefore this gene may be of use as a small molecule target for the treatment of any of the described diseases.

General_screening_panel_v1.4 Summary: Ag3402 The CG59220-01 gene represents a novel G-protein coupled receptor (GPCR) with highest expression in spinal cord sample (CT=31.12) and moderate expression in other samples from brain. Please see Panel CNS_neurodegeneration_v1.0 for discussion of utility of this gene in the central nervous system.

Low levels of expression of the CG59220-01 gene are also observed in areas outside of the central nervous system such as the, adipose tissue, fetal and adult heart, skeletal muscle, adrenal gland, pituitary gland, and thyroid suggesting the possibility of a wider role in intercellular signaling. Therapeutic modulation of the expression or function of this gene may therefore be useful in the treatment of metabolic disorders, including obesity and diabetes.

Panel 4D Summary: Ag3402 The CG59220-01 gene represents a novel G-protein coupled receptor (GPCR) with highest expression in colon (CT=33.12). Thus expression of this gene can be used to distinguish these samples from other samples used in this panel. In addition, expression of this gene is low/undetectable (CT values>35) in samples derived from IBD colitis and IBS Crohn's. Therefore, expression of this gene can be used to distinguish normal colon sample from the IBD colitis and IBD Crohn's sample used in this panel.

BN. CG59218-01: GPCR

Expression of gene CG59218-01 was assessed using the primer-probe set Ag3401, described in Table BNA. Results of the RTQ-PCR runs are shown in Tables BNB.

Table BNA. Probe Name Ag3401

TABLE BNA
Probe Name Ag3401

| | | | Start | SEQ ID |
|---|---|---|---|---|
| Primers | Sequences | Length | Position | NO: |
| Forward | 5′-gctggctactaggtttctcctt-3′ | 22 | 447 | 599 |
| Probe | TET-5′-atcatcatgcctgtcatcctgaccag-3′-TAMRA | 26 | 470 | 600 |
| Reverse | 5′-ttgatgtgggtatcacagaatg-3′ | 22 | 504 | 601 |

TABLE BNB
Panel 4D

| | Rel. Exp. (%) | | Rel. Exp. (%) |
|---|---|---|---|
| | Ag3401, Run | | Ag3401, Run |
| Tissue Name | 165825154 | Tissue Name | 165825154 |
| Secondary Th1 act | 2.4 | HUVEC IL-1beta | 0.0 |
| Secondary Th2 act | 3.4 | HUVEC IFN gamma | 0.0 |
| Secondary Tr1 act | 8.4 | HUVEC TNF alpha + | 0.0 |
| | | IFN gamma | |
| Secondary Th1 rest | 3.1 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 3.1 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 8.1 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 3.3 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 8.1 | Astrocytes rest | 5.7 |
| Secondary CD8 | 0.0 | Astrocytes TNF alpha + | 0.0 |
| lymphocyte rest | | IL-1beta | |
| Secondary CD8 | 1.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 5.7 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 3.1 | Liver cirrhosis | 100.0 |
| LAK cells IL-2 + IL-12 | 3.2 | Lupus kidney | 0.0 |
| LAK cells IL-2 + IFN | 8.0 | NCI-H292 none | 0.0 |
| gamma | | | |
| LAK cells IL-2 + IL-18 | 4.3 | NCI-H292 IL-4 | 0.0 |
| LAK cells | 2.1 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 3.3 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 3.4 | Lung fibroblast IFN | 0.0 |
| | | gamma | |
| B lymphocytes CD40L | 0.0 | Dermal fibroblast | 2.5 |
| and IL-4 | | CCD1070 rest | |
| EOL-1 dbcAMP | 0.0 | Dermal fibroblast | 14.8 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 6.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 17.1 |
| CD40 | | | |
| Monocytes rest | 0.0 | IBD Crohn's | 0.0 |
| Monocytes LPS | 6.1 | Colon | 0.0 |
| Macrophages rest | 0.0 | Lung | 0.0 |
| Macrophages LPS | 0.0 | Thymus | 0.0 |
| HUVEC none | 0.0 | Kidney | 3.2 |
| HUVEC starved | 0.0 | | |

CNS_neurodegeneration_v1.0 Summary: Ag3401 Expression of the CG59218-01 gene is low/undetectable (CTs>35) across all of the samples on this panel (data not shown).

General_screening_panel_v1.4 Summary: Ag3401 Expression of the CG59218-01 gene is low/undetectable (CTs>35) across all of the samples on this panel (data not shown). This gene product is most similar to members of the odorant receptor subfamily of GPCRs. Based on analogy to other odorant receptor genes, we predict that expression of this gene may be highest in nasal epithelium, a sample not represented on this panel.

Panel 4D Summary: Ag3401 Highest expression of the CG59218-01 gene is seen in the liver cirrhosis sample (CT=33.03). Thus, expression of this gene could be used to differentiate between this sample from the other samples on this panel and as a marker to detect the presence of liver cirrhosis. Furthermore, expression of this gene is not detected in normal liver in Panel 1.4, suggesting that its expression is unique to liver cirrhosis. This gene encodes a putative GPCR; therefore, antibodies or small molecule therapeutics could reduce or inhibit fibrosis that occurs in liver cirrhosis. In addition, antibodies to this putative GPCR could also be used for the diagnosis of liver cirrhosis.

BO. CG59211-01: GPCR

Expression of gene CG59211-01 was assessed using the primer-probe set Ag3397, described in Table BOA. Results of the RTQ-PCR runs are shown in Table BOB.

TABLE BOA

Probe Name A3397

| | | | Start | SEQ ID |
|---|---|---|---|---|
| Primers | Sequences | Length | Position | NO: |
| Forward | 5′-tcacaggcatcctagacttgac-3′ | 22 | 645 | 602 |
| Probe | TET-5′-tcatgtcctacatgttgatactgaaagca-3′-TAMRA | 29 | 675 | 603 |
| Reverse | 5′-tttcttgatgctatgctcaaca-3′ | 22 | 705 | 604 |

TABLE BOB
General_screening_panel_v1.4

| | Rel. | | Rel. |
|---|---|---|---|
| | Exp. (%) | | Exp. (%) |
| | Ag3397, | | Ag3397, |
| | Run | | Run |
| Tissue Name | 216822307 | Tissue Name | 216822307 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.7 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 0.6 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.5 |
| Breast ca. MCF-7 | 0.0 | Heart Pool | 0.5 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.6 | Thymus Pool | 0.0 |
| Trachea | 0.0 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.0 | CNS cancer | 0.8 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 2.6 | CNS cancer (glio) | 0.0 |
| | | SNB-19 | |
| Lung ca. SHP-77 | 100.0 | CNS cancer (glio) SF- | 0.0 |
| | | 295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 0.0 | Brain (Thalamus) Pool | 0.0 |
| Fetal Liver | 0.0 | Brain (whole) | 0.9 |
| Liver ca. HepG2 | 0.0 | Spinal Cord Pool | 0.0 |
| Kidney Pool | 0.9 | Adrenal Gland | 0.0 |
| Fetal Kidney | 3.3 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

CNS_neurodegeneration_v1.0 Summary: Ag3397 Expression of the CG59211-01 gene is low/undetectable (CT values>35) across the samples in this panel. (Data not shown.) This gene encodes a G protein-coupled receptor (GPCR), a type of cell surface receptor involved in signal transduction. It is most similar to members of the odorant receptor subfamily of GPCRs. Based on analogy to other odorant receptor genes, we predict that expression of this gene may be highest in nasal epithelium, a sample not represented on this panel.

General_screening_panel_v1.4 Summary: Ag3397 Significant expression of the CG59211-01 gene is seen exclusively in one of the lung cancer sample (CT=32.29). Therefore, expression of this gene may be used to distinguish this sample from other samples on this panel and as a marker for lung cancer. There is an increasing awareness that some GPCRs can regulate proliferative signaling pathways and that chronic stimulation or mutational activation of receptors can lead to oncogenic transformation. Activating mutations in GPCRs are associated with several types of human tumors and some receptors exhibit potent oncogenic activity due to agonist overexpression (Whitehead et al., 2001). Therefore, therapeutic modulation of the activity of the GPCR encoded by this gene may be beneficial in the treatment of lung cancer.

### REFERENCES

1. Whitehead I P, Zohn I E, Der C J. (2001) Rho GTPase-dependent transformation by G protein-coupled receptors. Oncogene Mar. 26, 2001;20(13):1547-55

Panel 4D Summary: Ag3397 Expression of the CG59211-01 gene is low/undetectable (CT values>35) across the samples in this panel. (Data not shown.)

BP. CG59276-01: Dihydroorotate Dehydrogenase

Expression of gene CG59276-01 was assessed using the primer-probe set Ag3524, described in Table BPA. Results of the RTQ-PCR runs are shown in Tables BPB, BPC, BPD, BPE and BPF.

TABLE BPA

Probe Name Ag3524

| Start | SEQ ID | | | |
|---|---|---|---|---|
| Primers | Sequences | Length | Position | NO: |
| Forward | 5′-ttcaggcactgttctttgactt-3′ | 22 | 1439 | 605 |
| Probe | TET-5′-aacagattttgcaacactttccaagg-3′-TAMRA | 26 | 1472 | 606 |
| Reverse | 5′-tgagggagtggtaacactgtgt-3′ | 22 | 1498 | 607 |

TABLE BPB
CNS_neurodegeneration_v1.0

| | Rel. | | Rel. |
|---|---|---|---|
| | Exp. (%) | | Exp. (%) |
| | Ag3524, | | Ag3524, |
| | Run | | Run |
| Tissue Name | 206915926 | Tissue Name | 206915926 |
| AD 1 Hippo | 26.8 | Control (Path) 3 | 18.9 |
| | | Temporal Ctx | |
| AD 2 Hippo | 30.8 | Control (Path) 4 | 51.4 |
| | | Temporal Ctx | |
| AD 3 Hippo | 25.3 | AD 1 Occipital Ctx | 37.1 |
| AD 4 Hippo | 38.2 | AD 2 Occipital Ctx | 0.0 |
| | | (Missing) | |
| AD 5 Hippo | 77.4 | AD 3 Occipital Ctx | 16.5 |
| AD 6 Hippo | 83.5 | AD 4 Occipital Ctx | 40.9 |
| Control 2 Hippo | 43.8 | AD 5 Occipital Ctx | 33.9 |
| Control 4 Hippo | 20.6 | AD 6 Occipital Ctx | 17.0 |
| Control (Path) 3 | 17.6 | Control 1 Occipital | 6.3 |
| Hippo | | Ctx | |
| AD 1 Temporal | 37.1 | Control 2 Occipital | 74.7 |
| Ctx | | Ctx | |
| AD 2 Temporal | 31.2 | Control 3 Occipital | 22.1 |
| Ctx | | Ctx | |
| AD 3 Temporal | 6.5 | Control 4 Occipital | 25.7 |
| Ctx | | Ctx | |
| AD 4 Temporal | 79.6 | Control (Path) 1 | 89.5 |
| Ctx | | Occipital Ctx | |
| AD 5 Inf Temporal | 97.9 | Control (Path) 2 | 18.4 |
| Ctx | | Occipital Ctx | |
| AD 5 Sup | 56.3 | Control (Path) 3 | 11.2 |
| Temporal Ctx | | Occipital Ctx | |
| AD 6 Inf Temporal | 100.0 | Control (Path) 4 | 24.8 |
| Ctx | | Occipital Ctx | |
| AD 6 Sup | 66.9 | Control 1 Parietal | 26.8 |
| Temporal Ctx | | Ctx | |
| Control 1 | 10.6 | Control 2 Parietal | 68.3 |
| Temporal Ctx | | Ctx | |
| Control 2 | 13.4 | Control 3 Parietal | 28.1 |
| Temporal Ctx | | Ctx | |
| Control 3 | 25.9 | Control (Path) 1 | 58.6 |
| Temporal Ctx | | Parietal Ctx | |
| Control 3 | 36.6 | Control (Path) 2 | 51.4 |
| Temporal Ctx | | Parietal Ctx | |
| Control (Path) 1 | 80.7 | Control (Path) 3 | 6.9 |
| Temporal Ctx | | Parietal Ctx | |
| Control (Path) 2 | 76.8 | Control (Path) 4 | 54.7 |
| Temporal Ctx | | Parietal Ctx | |

TABLE BPC
General_screening_panel_v1.4

| | Rel. | | Rel. |
|---|---|---|---|
| | Exp. (%) | | Exp. (%) |
| | Ag3524, | | Ag3524, |
| | Run | | Run |
| Tissue Name | 213390931 | Tissue Name | 213390931 |
| Adipose | 6.2 | Renal ca. TK-10 | 9.3 |
| Melanoma* | 3.1 | Bladder | 22.1 |
| Hs688(A).T | | | |
| Melanoma* | 9.2 | Gastric ca. (liver met.) | 24.8 |
| Hs688(B).T | | NCI-N87 | |
| Melanoma* M14 | 0.9 | Gastric ca. KATO III | 0.0 |
| Melanoma* | 0.4 | Colon ca. SW-948 | 1.2 |
| LOXIMVI | | | |
| Melanoma* SK- | 0.7 | Colon ca. SW480 | 4.2 |
| MEL-5 | | | |
| Squamous cell | 0.0 | Colon ca.* (SW480 | 10.5 |
| carcinoma SCC-4 | | met) SW620 | |
| Testis Pool | 11.3 | Colon ca. HT29 | 1.1 |
| Prostate ca.* (bone | 3.1 | Colon ca. HCT-116 | 5.3 |
| met) PC-3 | | | |
| Prostate Pool | 8.7 | Colon ca. CaCo-2 | 8.5 |
| Placenta | 1.3 | Colon cancer tissue | 7.2 |
| Uterus Pool | 2.9 | Colon ca. SW1116 | 1.4 |
| Ovarian ca. | 6.9 | Colon ca. Colo-205 | 1.6 |
| OVCAR-3 | | | |
| Ovarian ca. SK- | 11.7 | Colon ca. SW-48 | 1.7 |
| OV-3 | | | |
| Ovarian ca. | 0.2 | Colon Pool | 12.6 |
| OVCAR-4 | | | |
| Ovarian ca. | 8.5 | Small Intestine Pool | 20.9 |
| OVCAR-5 | | | |
| Ovarian ca. | 2.5 | Stomach Pool | 13.2 |
| IGROV-1 | | | |
| Ovarian ca. | 4.1 | Bone Marrow Pool | 8.7 |
| OVCAR-8 | | | |
| Ovary | 15.3 | Fetal Heart | 9.5 |
| Breast ca. MCF-7 | 3.4 | Heart Pool | 11.3 |
| Breast ca. MDA- | 3.1 | Lymph Node Pool | 27.2 |
| MB-231 | | | |
| Breast ca. BT 549 | 23.5 | Fetal Skeletal Muscle | 9.1 |
| Breast ca. T47D | 19.5 | Skeletal Muscle Pool | 6.7 |
| Breast ca. MDA-N | 0.3 | Spleen Pool | 8.9 |
| Breast Pool | 25.2 | Thymus Pool | 18.2 |
| Trachea | 12.6 | CNS cancer | 8.7 |
| | | (glio/astro) U87-MG | |
| Lung | 13.3 | CNS cancer | 7.5 |
| | | (glio/astro) U-118-MG | |
| Fetal Lung | 41.8 | CNS cancer | 21.3 |
| | | (neuro; met) SK-N-AS | |
| Lung ca. NCI-N417 | 0.0 | CNS cancer (astro) | 2.4 |
| | | SF-539 | |
| Lung ca. LX-1 | 32.1 | CNS cancer (astro) | 97.9 |
| | | SNB-75 | |
| Lung ca. NCI-H146 | 1.4 | CNS cancer (glio) | 3.0 |
| | | SNB-19 | |
| Lung ca. SHP-77 | 2.3 | CNS cancer (glio) SF- | 26.1 |
| | | 295 | |
| Lung ca. A549 | 5.4 | Brain (Amygdala) | 4.1 |
| | | Pool | |
| Lung ca. NCI-H526 | 0.4 | Brain (cerebellum) | 33.0 |
| Lung ca. NCI-H23 | 100.0 | Brain (fetal) | 25.5 |
| Lung ca. NCI-H460 | 7.9 | Brain (Hippocampus) | 7.5 |
| | | Pool | |
| Lung ca. HOP-62 | 4.2 | Cerebral Cortex Pool | 7.7 |
| Lung ca. NCI-H522 | 1.4 | Brain (Substantia | 5.8 |
| | | nigra) Pool | |
| Liver | 1.4 | Brain (Thalamus) Pool | 11.7 |
| Fetal Liver | 9.3 | Brain (whole) | 9.5 |
| Liver ca. HepG2 | 5.2 | Spinal Cord Pool | 10.5 |
| Kidney Pool | 40.3 | Adrenal Gland | 9.1 |
| Fetal Kidney | 40.1 | Pituitary gland Pool | 2.0 |
| Renal ca. 786-0 | 12.9 | Salivary Gland | 8.3 |
| Renal ca. A498 | 3.4 | Thyroid (female) | 2.5 |
| Renal ca. ACHN | 5.4 | Pancreatic ca. | 9.3 |
| | | CAPAN2 | |
| Renal ca. UO-31 | 7.2 | Pancreas Pool | 20.9 |

TABLE BPD
Panel 2D

| | Rel. Exp. (%) | | Rel. Exp. (%) |
|---|---|---|---|
| | Ag3524, | | Ag3524, |
| Tissue Name | Run 169590472 | Tissue Name | Run 169590472 |
| Normal Colon | 28.1 | Kidney Margin | 1.8 |
| | | 8120608 | |
| CC Well to Mod Diff | 2.4 | Kidney Cancer | 2.7 |
| (ODO3866) | | 8120613 | |
| CC Margin (ODO3866) | 1.4 | Kidney Margin | 8.0 |
| | | 8120614 | |
| CC Gr.2 rectosigmoid | 3.5 | Kidney Cancer | 6.0 |
| (ODO3868) | | 9010320 | |
| CC Margin (ODO3868) | 1.1 | Kidney Margin | 11.7 |
| | | 9010321 | |
| CC Mod Diff | 9.9 | Normal Uterus | 4.8 |
| (ODO3920) | | | |
| CC Margin (ODO3920) | 6.0 | Uterus Cancer | 2.3 |
| | | 064011 | |
| CC Gr.2 ascend colon | 12.3 | Normal Thyroid | 6.6 |
| (ODO3921) | | | |
| CC Margin (ODO3921) | 3.1 | Thyroid Cancer | 0.5 |
| | | 064010 | |
| CC from Partial | 12.8 | Thyroid Cancer | 2.1 |
| Hepatectomy | | A302152 | |
| (ODO4309) Mets | | | |
| Liver Margin | 34.6 | Thyroid Margin | 9.2 |
| (ODO4309) | | A302153 | |
| Colon mets to lung | 12.2 | Normal Breast | 13.6 |
| (OD04451-01) | | | |
| Lung Margin (OD04451- | 2.9 | Breast Cancer | 6.4 |
| 02) | | (OD04566) | |
| Normal Prostate 6546-1 | 9.9 | Breast Cancer | 9.8 |
| | | (OD04590-01) | |
| Prostate Cancer | 16.6 | Breast Cancer Mets | 14.0 |
| (OD04410) | | (OD04590-03) | |
| Prostate Margin | 15.4 | Breast Cancer | 5.3 |
| (OD04410) | | Metastasis | |
| | | (OD04655-05) | |
| Prostate Cancer | 20.6 | Breast Cancer | 11.2 |
| (OD04720-01) | | 064006 | |
| Prostate Margin | 16.6 | Breast Cancer 1024 | 31.6 |
| (OD04720-02) | | | |
| Normal Lung 061010 | 27.2 | Breast Cancer | 8.0 |
| | | 9100266 | |
| Lung Met to Muscle | 3.5 | Breast Margin | 4.6 |
| (ODO4286) | | 9100265 | |
| Muscle Margin | 6.7 | Breast Cancer | 4.1 |
| (ODO4286) | | A209073 | |
| Lung Malignant Cancer | 3.7 | Breast Margin | 8.4 |
| (OD03126) | | A209073 | |
| Lung Margin (OD03126) | 10.8 | Normal Liver | 100.0 |
| Lung Cancer (OD04404) | 4.1 | Liver Cancer 064003 | 44.4 |
| Lung Margin (OD04404) | 4.3 | Liver Cancer 1025 | 47.0 |
| Lung Cancer (OD04565) | 0.9 | Liver Cancer 1026 | 0.9 |
| Lung Margin (OD04565) | 8.5 | Liver Cancer 6004-T | 59.5 |
| Lung Cancer (OD04237- | 13.2 | Liver Tissue 6004-N | 6.6 |
| 01) | | | |
| Lung Margin (OD04237- | 4.5 | Liver Cancer 6005-T | 1.5 |
| 02) | | | |
| Ocular Mel Met to Liver | 6.7 | Liver Tissue 6005-N | 0.5 |
| (ODO4310) | | | |
| Liver Margin | 6.7 | Normal Bladder | 21.5 |
| (ODO4310) | | | |
| Melanoma Mets to Lung | 3.9 | Bladder Cancer 1023 | 0.9 |
| (OD04321) | | | |
| Lung Margin (OD04321) | 4.7 | Bladder Cancer | 5.9 |
| | | A302173 | |
| Normal Kidney | 35.8 | Bladder Cancer | 5.5 |
| | | (OD04718-01) | |
| Kidney Ca, Nuclear | 18.0 | Bladder Normal | 6.5 |
| grade 2 (OD04338) | | Adjacent (OD04718- | |
| | | 03) | |
| Kidney Margin | 14.1 | Normal Ovary | 4.5 |
| (OD04338) | | | |
| Kidney Ca Nuclear | 13.7 | Ovarian Cancer | 3.7 |
| grade 1/2 (OD04339) | | 064008 | |
| Kidney Margin | 9.9 | Ovarian Cancer | 11.0 |
| (OD04339) | | (OD04768-07) | |
| Kidney Ca, Clear cell | 10.8 | Ovary Margin | 1.1 |
| type (OD04340) | | (OD04768-08) | |
| Kidney Margin | 12.7 | Normal Stomach | 7.6 |
| (OD04340) | | | |
| Kidney Ca, Nuclear | 0.5 | Gastric Cancer | 3.3 |
| grade 3 (OD04348) | | 9060358 | |
| Kidney Margin | 10.6 | Stomach Margin | 4.0 |
| (OD04348) | | 9060359 | |
| Kidney Cancer | 0.9 | Gastric Cancer | 8.2 |
| (OD04622-01) | | 9060395 | |
| Kidney Margin | 1.6 | Stomach Margin | 5.5 |
| (OD04622-03) | | 9060394 | |
| Kidney Cancer | 4.2 | Gastric Cancer | 3.8 |
| (OD04450-01) | | 9060397 | |
| Kidney Margin | 5.4 | Stomach Margin | 0.9 |
| (OD04450-03) | | 9060396 | |
| Kidney Cancer 8120607 | 1.0 | Gastric Cancer | 14.5 |
| | | 064005 | |

TABLE BPE
Panel 4D

| | Rel. Exp. (%) | | Rel. Exp. (%) |
|---|---|---|---|
| | Ag3524, Run | | Ag3524, Run |
| Tissue Name | 166445583 | Tissue Name | 166445583 |
| Secondary Th1 act | 13.9 | HUVEC IL-1beta | 2.4 |
| Secondary Th2 act | 17.1 | HUVEC IFN gamma | 16.4 |
| Secondary Tr1 act | 15.4 | HUVEC TNF alpha + | 4.8 |
| | | IFN gamma | |
| Secondary Th1 rest | 36.6 | HUVEC TNF alpha + | 3.8 |
| | | IL4 | |
| Secondary Th2 rest | 19.1 | HUVEC IL-11 | 6.1 |
| Secondary Tr1 rest | 28.7 | Lung Microvascular EC | 9.2 |
| | | none | |
| Primary Th1 act | 10.2 | Lung Microvascular EC | 5.0 |
| | | TNF alpha + IL-1beta | |
| Primary Th2 act | 18.2 | Microvascular Dermal | 14.5 |
| | | EC none | |
| Primary Tr1 act | 28.5 | Microsvasular Dermal | 10.4 |
| | | EC TNF alpha + IL-1beta | |
| Primary Th1 rest | 100.0 | Bronchial epithelium | 6.1 |
| | | TNF alpha + IL-1beta | |
| Primary Th2 rest | 42.3 | Small airway epithelium | 3.3 |
| | | none | |
| Primary Tr1 rest | 35.8 | Small airway epithelium | 29.3 |
| | | TNF alpha + IL-1beta | |
| CD45RA CD4 | 14.6 | Coronery artery SMC rest | 5.6 |
| lymphocyte act | | | |
| CD45RO CD4 | 28.7 | Coronery artery SMC | 3.4 |
| lymphocyte act | | TNF alpha + IL-1beta | |
| CD8 lymphocyte act | 17.7 | Astrocytes rest | 12.1 |
| Secondary CD8 | 23.7 | Astrocytes TNF alpha + | 11.5 |
| lymphocyte rest | | IL-1beta | |
| Secondary CD8 | 12.7 | KU-812 (Basophil) rest | 23.2 |
| lymphocyte act | | | |
| CD4 lymphocyte none | 22.2 | KU-812 (Basophil) | 37.1 |
| | | PMA/ionomycin | |
| 2ry Th1/Th2/Tr1_anti- | 39.5 | CCD1106 | 5.0 |
| CD95 CH11 | | (Keratinocytes) none | |
| LAK cells rest | 17.9 | CCD1106 | 41.5 |
| | | (Keratinocytes) | |
| | | TNF alpha + IL-1beta | |
| LAK cells IL-2 | 41.2 | Liver cirrhosis | 37.9 |
| LAK cells IL-2 + IL-12 | 31.0 | Lupus kidney | 21.8 |
| LAK cells IL-2 + IFN | 47.0 | NCI-H292 none | 25.0 |
| gamma | | | |
| LAK cells IL-2 + IL-18 | 44.8 | NCI-H292 IL-4 | 21.3 |
| LAK cells | 5.3 | NCI-H292 IL-9 | 21.6 |
| PMA/ionomycin | | | |
| NK Cells IL-2 rest | 17.8 | NCI-H292 IL-13 | 9.2 |
| Two Way MLR 3 day | 47.6 | NCI-H292 IFN gamma | 10.6 |
| Two Way MLR 5 day | 13.0 | HPAEC none | 16.5 |
| Two Way MLR 7 day | 18.8 | HPAEC TNF alpha + IL- | 4.0 |
| | | 1beta | |
| PBMC rest | 7.6 | Lung fibroblast none | 30.4 |
| PBMC PWM | 24.7 | Lung fibroblast TNF | 24.3 |
| | | alpha + IL-1beta | |
| PBMC PHA-L | 4.9 | Lung fibroblast IL-4 | 23.8 |
| Ramos (B cell) none | 19.5 | Lung fibroblast IL-9 | 14.6 |
| Ramos (B cell) | 10.7 | Lung fibroblast IL-13 | 17.1 |
| ionomycin | | | |
| B lymphocytes PWM | 22.8 | Lung fibroblast IFN | 24.1 |
| | | gamma | |
| B lymphocytes CD40L | 40.1 | Dermal fibroblast | 20.3 |
| and IL-4 | | CCD1070 rest | |
| EOL-1 dbcAMP | 15.9 | Dermal fibroblast | 22.4 |
| | | CCD1070 TNF alpha | |
| EOL-1 dbcAMP | 11.7 | Dermal fibroblast | 0.6 |
| PMA/ionomycin | | CCD1070 IL-1beta | |
| Dendritic cells none | 22.2 | Dermal fibroblast IFN | 10.6 |
| | | gamma | |
| Dendritic cells LPS | 14.5 | Dermal fibroblast IL-4 | 11.8 |
| Dendritic cells anti- | 23.5 | IBD Colitis 2 | 7.3 |
| CD40 | | | |
| Monocytes rest | 37.9 | IBD Crohn's | 8.0 |
| Monocytes LPS | 15.8 | Colon | 54.0 |
| Macrophages rest | 10.2 | Lung | 9.7 |
| Macrophages LPS | 5.0 | Thymus | 39.5 |
| HUVEC none | 17.3 | Kidney | 63.7 |
| HUVEC starved | 11.8 | | |

TABLE BPF
Panel 5 Islet

| | Rel. Exp. (%) | | Rel. Exp. (%) |
|---|---|---|---|
| | Ag3524, Run | | Ag3524, Run |
| Tissue Name | 242386392 | Tissue Name | 242386392 |
| 97457_Patient- | 66.0 | 94709_Donor 2 AM - A_adipose | 9.0 |
| 02go_adipose | | | |
| 97476_Patient- | 10.7 | 94710_Donor 2 AM - B_adipose | 9.8 |
| 07sk_skeletal muscle | | | |
| 97477_Patient- | 9.4 | 94711_Donor 2 AM - C_adipose | 8.1 |
| 07ut_uterus | | | |
| 97478_Patient- | 7.8 | 94712_Donor 2 AD - A_adipose | 17.8 |
| 07pl_placenta | | | |
| 99167_Bayer Patient | 10.2 | 94713_Donor 2 AD - B_adipose | 27.0 |
| 1 | | | |
| 97482_Patient- | 0.0 | 94714_Donor 2 AD - C_adipose | 43.5 |
| 08ut_uterus | | | |
| 97483_Patient- | 0.0 | 94742_Donor 3 U - | 0.0 |
| 08pl_placenta | | A_Mesenchymal Stem Cells | |
| 97486_Patient- | 0.0 | 94743_Donor 3 U - | 0.0 |
| 09sk_skeletal muscle | | B_Mesenchymal Stem Cells | |
| 97487_Patient- | 0.0 | 94730_Donor 3 AM - A_adipose | 16.8 |
| 09ut_uterus | | | |
| 97488_Patient- | 17.0 | 94731_Donor 3 AM - B_adipose | 7.8 |
| 09pl_placenta | | | |
| 97492_Patient- | 28.1 | 94732_Donor 3 AM - C_adipose | 18.0 |
| 10ut_uterus | | | |
| 97493_Patient- | 25.3 | 94733_Donor 3 AD - A_adipose | 0.0 |
| 10pl_placenta | | | |
| 97495_Patient- | 61.6 | 94734_Donor 3 AD - B_adipose | 0.0 |
| 11go_adipose | | | |
| 97496_Patient- | 28.5 | 94735_Donor 3 AD - C_adipose | 18.9 |
| 11sk_skeletal muscle | | | |
| 97497_Patient- | 16.2 | 77138_Liver_HepG2untreated | 15.8 |
| 11ut_uterus | | | |
| 97498_Patient- | 5.0 | 73556_Heart_Cardiac stromal | 9.7 |
| 11pl_placenta | | cells (primary) | |
| 97500_Patient- | 100.0 | 81735_Small Intestine | 62.9 |
| 12go_adipose | | | |
| 97501_Patient- | 42.6 | 72409_Kidney_Proximal | 24.0 |
| 12sk_skeletal muscle | | Convoluted Tubule | |
| 97502_Patient- | 41.8 | 82685_Small | 32.8 |
| 12ut_uterus | | intestine_Duodenum | |
| 97503_Patient- | 0.0 | 90650_Adrenal_Adrenocortical | 0.0 |
| 12pl_placenta | | adenoma | |
| 94721_Donor 2 U - | 0.0 | 72410_Kidney_HRCE | 39.8 |
| A_Mesenchymal | | | |
| Stem Cells | | | |
| 94722_Donor 2 U - | 0.0 | 72411_Kidney_HRE | 21.8 |
| B_Mesenchymal | | | |
| Stem Cells | | | |
| 94723_Donor 2 U - | 0.0 | 73139_Uterus_Uterine smooth | 8.1 |
| C_Mesenchymal | | muscle cells | |
| Stem Cells | | | |

CNS_neurodegeneration_v1.0 Summary: Ag3524 No differential expression of the CG59276-01 gene is detected between Alzheimer's diseased postmortem brains and those of non-demented controls in this experiment. However, as observed in panel 1.4 this gene is expressed at low levels throughout the CNS, including in amygdala, substantia nigra, thalamus, cerebellum, cerebral cortex, and spinal cord. Therefore, this gene may play a role in central nervous system disorders such as Parkinson's disease, epilepsy, multiple sclerosis, schizophrenia and depression.

General_screening_panel_v1.4 Summary: Ag3524 Expression of the CG59276-01 gene is highest in a sample derived from a brain and lung cancer cell lines (CTs=29). Thus, the expression of this gene could be used to distinguish these samples from the other samples in the panel. The CG59276-01 gene encodes a dihydroorotate dehydrogenase (DHODH) homolog. DHODH is an enzyme involved in the pathway for pyrimidine production. Drugs known to inhibit DHODH activity, such as brequinar sodium (Dup-785), have been shown to have anti-tumor activities (ref. 1). Therefore, therapeutic modulation of the activity of this gene encoded by this gene may be beneficial in the treatment of CNS and lung cancer. In addition, low to moderate expression of this gene is seen in all of the samples on this panel. Therefore, this gene may be playing an important role in cellular function.

This gene is expressed at low to moderate levels in a number of tissues with metabolic or endocrine function, including adipose, adrenal gland, gastrointestinal tract, pancreas, and skeletal muscle. Therefore, therapeutic modulation of the activity of this gene may prove useful in the treatment of endocrine/metabolically related diseases, such as obesity and diabetes.

Recently, it has been demonstrated that down regulation of DHODH mRNA using RNA interference (RNAi) may inhibit growth of Plasmodium falciparum (ref 2). REFERENCES

1. Braakhuis B J, van Dongen G A, Peters G J, van Walsum M, Snow G B (1990) Antitumor activity of brequinar sodium (Dup-785) against human head and neck squamous cell carcinoma xenografts. Cancer Lett 49(2):133-7.

2. McRobert L, McConkey G A.(2002) RNA interference (RNAi) inhibits growth of Plasmodium falciparum. Mol Biochem Parasitol 119(2):273-8

Panel 2D Summary: Ag3524 The expression of this gene appears to be highest in a sample derived from a normal liver tissue (CT=30.3). In addition, there appears to be substantial expression in other samples derived from liver cancers and breast cancers. Thus, the expression of this gene could be used to distinguish normal liver tissue from other samples in the panel. Moreover, therapeutic modulation of this gene, through the use of small molecule drugs, protein therapeutics or antibodies could be of benefit in the treatment of liver or breast cancer.

Panel 4D Summary: Ag3524 Highest expression of the CG59276-01 gene is detected in resting primary Th1 cells (CT=30.03). In addition, the expression of this gene is significantly reduced in activated primary Th1 cells, suggesting a regulatory role for this gene in T-cell activation. The CG59276-01 encodes a dihydroorotate dehydrogenase, an enzyme involved in the pathway for pyrimidine production. Recently, an inhibitor of this enzyme, leflunomide has been shown to be an effective treatment for rheumatoid arthritis (ref 1). Therefore, therapeutics designed with the protein encoded for by this transcript could be important in regulating T cell function and treating T cell mediated diseases such as asthma, rheumatoid arthritis, psoriasis, IBD, and systemic lupus erythematosus.

Overall, this gene is expressed at low to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### REFERENCES

1. Schattenkirchner M. (2000) The use of leflunomide in the treatment of rheumatoid arthritis: an experimental and clinical review. Immunopharmacology 47(2-3):291-3

Panel 5 Islet Summary: Ag3524 This gene has a low level of expression in adipose tissue (CTs=33-35). Thus, this gene product may be a small molecule drug for the treatment of obesity and obesity-related diseases, including Type 2 diabetes.

BQ. CG59268-01: K1AA2372

Expression of gene CG59268-01 was assessed using the primer-probe set Ag3523, described in Table BQA. Results of the RTQ-PCR runs are shown in Tables BQB and BQC.

TABLE BQA

Probe Name Ag3523

| | | | Start | SEQ ID |
|---|---|---|---|---|
| Primers | Sequences | Length | Position | NO: |
| Forward | 5′-tactcttttggcttgatggaaa-3′ | 22 | 556 | 608 |
| Probe | TET-5′-ccaacttctacgaccaggcagaaaa-3′-TAMRA | 25 | 578 | 609 |
| Reverse | 5′-gacaaagagttggtgcctcttt-3′ | 22 | 610 | 610 |

TABLE BQB
General_screening_panel_v1.4

| | Rel. Exp. | | Rel. Exp. |
|---|---|---|---|
| | (%) Ag3523, | | (%) Ag3523, |
| | Run | | Run |
| Tissue Name | 216874716 | Tissue Name | 216874716 |
| Adipose | 47.6 | Renal ca. TK-10 | 62.9 |
| Melanoma* | 4.0 | Bladder | 25.2 |
| Hs688(A).T | | | |
| Melanoma* | 0.0 | Gastric ca. (liver met.) | 40.1 |
| Hs688(B).T | | NCI-N87 | |
| Melanoma* M14 | 15.4 | Gastric ca. KATO III | 0.0 |
| Melanoma* | 0.8 | Colon ca. SW-948 | 0.0 |
| LOXIMVI | | | |
| Melanoma* SK- | 39.5 | Colon ca. SW480 | 9.7 |
| MEL-5 | | | |
| Squamous cell | 0.0 | Colon ca.* (SW480 | 10.2 |
| carcinoma SCC-4 | | met) SW620 | |
| Testis Pool | 35.8 | Colon ca. HT29 | 27.2 |
| Prostate ca.* (bone | 8.5 | Colon ca. HCT-116 | 52.5 |
| met) PC-3 | | | |
| Prostate Pool | 0.0 | Colon ca. CaCo-2 | 31.2 |
| Placenta | 0.0 | Colon cancer tissue | 14.1 |
| Uterus Pool | 0.0 | Colon ca. SW1116 | 0.0 |
| Ovarian ca. | 0.0 | Colon ca. Colo-205 | 12.2 |
| OVCAR-3 | | | |
| Ovarian ca. SK- | 16.3 | Colon ca. SW-48 | 0.0 |
| OV-3 | | | |
| Ovarian ca. | 10.3 | Colon Pool | 0.0 |
| OVCAR-4 | | | |
| Ovarian ca. | 47.6 | Small Intestine Pool | 3.9 |
| OVCAR-5 | | | |
| Ovarian ca. | 0.0 | Stomach Pool | 4.3 |
| IGROV-1 | | | |
| Ovarian ca. | 1.5 | Bone Marrow Pool | 0.0 |
| OVCAR-8 | | | |
| Ovary | 0.0 | Fetal Heart | 4.2 |
| Breast ca. MCF-7 | 6.0 | Heart Pool | 4.2 |
| Breast ca. MDA- | 8.8 | Lymph Node Pool | 0.0 |
| MB-231 | | | |
| Breast ca. BT 549 | 33.4 | Fetal Skeletal Muscle | 0.0 |
| Breast ca. T47D | 62.9 | Skeletal Muscle Pool | 5.1 |
| Breast ca. MDA-N | 8.8 | Spleen Pool | 0.0 |
| Breast Pool | 0.0 | Thymus Pool | 0.0 |
| Trachea | 0.0 | CNS cancer | 9.0 |
| | | (glio/astro) U87-MG | |
| Lung | 0.0 | CNS cancer | 33.2 |
| | | (glio/astro) U-118-MG | |
| Fetal Lung | 15.1 | CNS cancer | 23.5 |
| | | (neuro; met) SK-N-AS | |
| Lung ca. NCI-N417 | 3.5 | CNS cancer (astro) | 11.0 |
| | | SF-539 | |
| Lung ca. LX-1 | 3.7 | CNS cancer (astro) | 13.8 |
| | | SNB-75 | |
| Lung ca. NCI-H146 | 0.0 | CNS cancer (glio) | 20.7 |
| | | SNB-19 | |
| Lung ca. SHP-77 | 11.0 | CNS cancer (glio) SF- | 30.1 |
| | | 295 | |
| Lung ca. A549 | 6.3 | Brain (Amygdala) | 0.0 |
| | | Pool | |
| Lung ca. NCI-H526 | 0.0 | Brain (cerebellum) | 0.0 |
| Lung ca. NCI-H23 | 3.8 | Brain (fetal) | 4.5 |
| Lung ca. NCI-H460 | 13.5 | Brain (Hippocampus) | 4.6 |
| | | Pool | |
| Lung ca. HOP-62 | 4.0 | Cerebral Cortex Pool | 0.0 |
| Lung ca. NCI-H522 | 9.2 | Brain (Substantia | 4.4 |
| | | nigra) Pool | |
| Liver | 4.5 | Brain (Thalamus) Pool | 0.0 |
| Fetal Liver | 0.0 | Brain (whole) | 0.0 |
| Liver ca. HepG2 | 100.0 | Spinal Cord Pool | 0.0 |
| Kidney Pool | 6.5 | Adrenal Gland | 8.3 |
| Fetal Kidney | 17.9 | Pituitary gland Pool | 6.6 |
| Renal ca. 786-0 | 38.7 | Salivary Gland | 16.3 |
| Renal ca. A498 | 3.2 | Thyroid (female) | 0.0 |
| Renal ca. ACHN | 9.0 | Pancreatic ca. | 16.5 |
| | | CAPAN2 | |
| Renal ca. UO-31 | 0.0 | Pancreas Pool | 11.3 |

TABLE BQC
Panel 4D

| | Rel. Exp. (%) | | Rel. Exp. (%) |
|---|---|---|---|
| | Ag3523, Run | | Ag3523, Run |
| Tissue Name | 166407138 | Tissue Name | 166407138 |
| Secondary Th1 act | 11.3 | HUVEC IL-1beta | 1.5 |
| Secondary Th2 act | 2.7 | HUVEC IFN gamma | 0.0 |
| Secondary Tr1 act | 9.5 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 1.3 | HUVEC IL-11 | 5.1 |
| Secondary Tr1 rest | 1.7 | Lung Microvascular EC | 0.0 |
| | | none | |
| Primary Th1 act | 13.1 | Lung Microvascular EC | 0.0 |
| | | TNF alpha + IL-1beta | |
| Primary Th2 act | 10.8 | Microvascular Dermal | 1.0 |
| | | EC none | |
| Primary Tr1 act | 7.5 | Microsvasular Dermal | 1.6 |
| | | EC TNF alpha + IL-1beta | |
| Primary Th1 rest | 1.4 | Bronchial epithelium | 0.0 |
| | | TNF alpha + IL-1beta | |
| Primary Th2 rest | 0.0 | Small airway epithelium | 1.0 |
| | | none | |
| Primary Tr1 rest | 2.6 | Small airway epithelium | 4.4 |
| | | TNF alpha + IL-1beta | |
| CD45RA CD4 | 0.0 | Coronery artery SMC rest | 0.0 |
| lymphocyte act | | | |
| CD45RO CD4 | 8.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 1.5 | Astrocytes TNF alpha + | 0.0 |
| lymphocyte rest | | IL-1beta | |
| Secondary CD8 | 3.1 | KU-812 (Basophil) rest | 0.0 |
| lymphocyte act | | | |
| CD4 lymphocyte none | 1.0 | KU-812 (Basophil) | 8.1 |
| | | PMA/ionomycin | |
| 2ry Th1/Th2/Tr1_anti- | 3.7 | CCD1106 | 4.0 |
| CD95 CH11 | | (Keratinocytes) none | |
| LAK cells rest | 7.2 | CCD1106 | 2.2 |
| | | (Keratinocytes) | |
| | | TNF alpha + IL-1beta | |
| LAK cells IL-2 | 0.0 | Liver cirrhosis | 15.3 |
| LAK cells IL-2 + IL-12 | 8.9 | Lupus kidney | 5.0 |
| LAK cells IL-2 + IFN | 12.2 | NCI-H292 none | 1.0 |
| gamma | | | |
| LAK cells IL-2 + IL-18 | 4.7 | NCI-H292 IL-4 | 0.6 |
| LAK cells | 1.4 | NCI-H292 IL-9 | 1.6 |
| PMA/ionomycin | | | |
| NK Cells IL-2 rest | 7.9 | NCI-H292 IL-13 | 1.5 |
| Two Way MLR 3 day | 0.0 | NCI-H292 IFN gamma | 1.2 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 5.5 | Lung fibroblast none | 0.0 |
| PBMC PWM | 2.6 | Lung fibroblast TNF | 0.0 |
| | | alpha + IL-1beta | |
| PBMC PHA-L | 0.0 | Lung fibroblast IL-4 | 3.9 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 8.2 | Lung fibroblast IFN | 0.0 |
| | | gamma | |
| B lymphocytes CD40L | 1.5 | Dermal fibroblast | 0.0 |
| and IL-4 | | CCD1070 rest | |
| EOL-1 dbcAMP | 4.5 | Dermal fibroblast | 13.9 |
| | | CCD1070 TNF alpha | |
| EOL-1 dbcAMP | 10.8 | Dermal fibroblast | 0.4 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 11.5 | IBD Crohn's | 66.0 |
| Monocytes LPS | 6.4 | Colon | 100.0 |
| Macrophages rest | 4.5 | Lung | 0.0 |
| Macrophages LPS | 1.4 | Thymus | 38.4 |
| HUVEC none | 1.9 | Kidney | 0.0 |
| HUVEC starved | 1.6 | | |

CNS_neurodegeneration_v1.0 Summary: Ag3523 Expression of this gene is low/undetectable (CTs>35) across all of the samples on this panel (data not shown).

General_screening_panel_v1.4 Summary: Ag3523 Expression of the CG59268-01 gene is highest in sample derived from liver cancer cell line (CT=32.55). Therefore, expression of this gene may be used to distinguish liver cancers from the other samples on this panel. In addition, low levels of expression of this gene are also observed in one of the ovarian cancer, 2 of the breast cancer, 2 of the renal cancer, bladder, gastric cancer, 3 of the colon cancer, and 4 of the CNS cancer samples. Therefore, therapeutic modulation of the activity of this gene product may be beneficial in the treatment of these cancers.

Among the tissues with metabolic or endocrine function, this gene is expressed at low levels in adipose tissue sample. Adipose tissue has several crucial roles including (i) mobilization from stores of fatty acids as an energy source, (ii) catabolism of lipoproteins such as very-low-density lipoprotein and (iii) synthesis and release of hormonal signals such as leptin and interleukin-6 (Coppack et al., 2001). Therefore, therapeutic modulation of the activity of this gene may prove useful in the treatment of endocrine/metabolically related diseases, such as obesity, hyperlipidemia, and insulin resistance.

### REFERENCES

1. Coppack S W, Patel J N, Lawrence V J. (2001) Nutritional regulation of lipid metabolism in human adipose tissue. Exp Clin Endocrinol Diabetes; 109(Suppl 2):S202-S214

Panel 4D Summary: Ag3523 Expression of the CG59268-01 gene is highest in sample derived from colon (CT=31.56). Therefore, expression of this gene may be used to distinguish colon sample from the other samples on this panel. In addition, significant expression of this gene is also observed in IBD Crohn's sample (CT=32.16). Thus, expression of this gene in colon and Crohn's sample can be used to distinguish these two samples from IBD Colitis 2 sample. In addition, therapeutic modulation of the activity of this gene product may be beneficial in the treatment of IBD Crohn's disease.

BR. CG59549-01: H326 Like

Expression of gene CG59549-01 was assessed using the primer-probe set Ag3464, described in Table BRA. Results of the RTQ-PCR runs are shown in Tables BRB and BRC.

TABLE BRA

Probe Name Ag3464

| | | | Start | SEQ ID |
|---|---|---|---|---|
| Primers | Sequences | Length | Position | NO: |
| Forward | 5′-gtgcgtcacctgttacagaga-3′ | 21 | 1678 | 611 |
| Probe | TET-5′-ctcatcaacccggctggagagatcat-3′-TAMRA | 26 | 1700 | 612 |
| Reverse | 5′-ctcttcttcatctgggaactca-3′ | 22 | 1731 | 613 |

TABLE BRB
General_screening_panel_v1.4

| | Rel. Exp. | | Rel. Exp. |
|---|---|---|---|
| | (%) Ag3464, | | (%) Ag3464, |
| | Run | | Run |
| Tissue Name | 217067408 | Tissue Name | 217067408 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 1.6 |
| Hs688(B).T | | NCI-N87 | |
| Melanoma* M14 | 1.0 | Gastric ca. KATO III | 2.1 |
| Melanoma* | 0.0 | Colon ca. SW-948 | 0.0 |
| LOXIMVI | | | |
| Melanoma* SK- | 23.8 | Colon ca. SW480 | 2.6 |
| MEL-5 | | | |
| Squamous cell | 0.0 | Colon ca.* (SW480 | 0.0 |
| carcinoma SCC-4 | | met) SW620 | |
| Testis Pool | 22.8 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.3 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 1.0 | Colon ca. SW-48 | 0.0 |
| OV-3 | | | |
| Ovarian ca. | 0.0 | Colon Pool | 3.3 |
| OVCAR-4 | | | |
| Ovarian ca. | 0.0 | Small Intestine Pool | 0.9 |
| OVCAR-5 | | | |
| Ovarian ca. | 0.0 | Stomach Pool | 0.0 |
| IGROV-1 | | | |
| Ovarian ca. | 4.8 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 4.9 | Skeletal Muscle Pool | 0.0 |
| Breast ca. MDA-N | 1.1 | Spleen Pool | 0.0 |
| Breast Pool | 2.2 | Thymus Pool | 0.7 |
| Trachea | 0.0 | CNS cancer | 0.0 |
| | | (glio/astro) U87-MG | |
| Lung | 0.0 | CNS cancer | 20.4 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 2.0 | CNS cancer (glio) | 0.0 |
| | | SNB-19 | |
| Lung ca. SHP-77 | 0.7 | CNS cancer (glio) SF- | 100.0 |
| | | 295 | |
| Lung ca. A549 | 0.6 | Brain (Amygdala) | 1.1 |
| | | Pool | |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 2.6 | Cerebral Cortex Pool | 0.0 |
| Lung ca. NCI-H522 | 0.0 | Brain (Substantia | 0.8 |
| | | nigra) Pool | |
| Liver | 0.0 | Brain (Thalamus) Pool | 1.1 |
| Fetal Liver | 0.0 | Brain (whole) | 0.0 |
| Liver ca. HepG2 | 0.0 | Spinal Cord Pool | 0.8 |
| Kidney Pool | 0.0 | Adrenal Gland | 2.0 |
| Fetal Kidney | 3.2 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 1.8 |

TABLE BRC
Panel 4D

| | Rel. Exp. (%) | | Rel. Exp. (%) |
|---|---|---|---|
| | Ag3464, Run | | Ag3464, Run |
| Tissue Name | 166417099 | Tissue Name | 166417099 |
| Secondary Th1 act | 0.0 | HUVEC IL-1beta | 0.0 |
| Secondary Th2 act | 0.0 | HUVEC IFN gamma | 0.0 |
| Secondary Tr1 act | 2.5 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL-1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 12.0 |
| | | PMA/ionomycin | |
| 2ry Th1/Th2/Tr1_anti- | 5.9 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 10.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 3.2 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 7.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 0.0 | IBD Crohn's | 6.3 |
| Monocytes LPS | 0.0 | Colon | 27.4 |
| Macrophages rest | 0.0 | Lung | 3.8 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag3464 Expression of the CG59549-01 gene is low/undetectable (CTs>35) across all of the samples on this panel (data not shown).

General_screening_panel_v1.4 Summary: Ag3464 Expression of the CG59549-01 gene is highest in a CNS cancer (glio) SF-295 sample (CT=31.15). Thus, the expression of this gene could be used to distinguish this sample from the other samples in the panel. In addition, low to moderate expression of this gene is detected in a melanoma and a CNS cancer sample. Therefore, therapeutic modulation of this gene or its protein product may be beneficial in the treatment of melanoma and CNS cancer.

Panel 4D Summary: Ag3464 Low but significant expression of the CG59549-01 gene is detected exclusively in liver cirrhosis sample (CT=33.4). Therefore, expression of this gene may be used to distinguish liver cirrhosis from the other samples on this panel. Furthermore, expression of this gene is not detected in normal liver in Panel 1.3D, suggesting that its expression is unique to liver cirrhosis. Therefore, antibodies or small molecule therapeutics could reduce or inhibit fibrosis that occurs in liver cirrhosis. In addition, antibodies to this gene product could also be used for the diagnosis of liver cirrhosis.

BS. CG59641-01: Acetyl-Coa Carboxylase 2

Expression of gene CG59641-01 was assessed using the primer-probe set Ag3502, described in Table BSA. Results of the RTQ-PCR runs are shown in Table BSB.

TABLE BSA

Probe Name Ag3502

| | | | Start | SEQ ID |
|---|---|---|---|---|
| Primers | Sequences | Length | Position | NO: |
| Forward | 5′-ctccctacgtcaccaaggat-3′ | 20 | 5090 | 614 |
| Probe | TET-5′-aagcgattccaggcccagaccct-3′-TAMRA | 23 | 5122 | 615 |
| Reverse | 5′-ccgggaagtcatagatgtaggt-3′- | 22 | 5152 | 616 |

TABLE BSB
General_screening_panel_v1.4

| | Rel. Exp. | | Rel. Exp. |
|---|---|---|---|
| | (%) Ag3502, | | (%) Ag3502, |
| | Run | | Run |
| Tissue Name | 217131537 | Tissue Name | 217131537 |
| Adipose | 100.0 | Renal ca. TK-10 | 6.6 |
| Melanoma* | 1.5 | Bladder | 14.2 |
| Hs688(A).T | | | |
| Melanoma* | 1.4 | Gastric ca. (liver met.) | 11.6 |
| Hs688(B).T | | NCI-N87 | |
| Melanoma* M14 | 7.2 | Gastric ca. KATO III | 4.2 |
| Melanoma* | 0.0 | Colon ca. SW-948 | 0.5 |
| LOXIMVI | | | |
| Melanoma* SK- | 14.8 | Colon ca. SW480 | 7.8 |
| MEL-5 | | | |
| Squamous cell | 0.2 | Colon ca.* (SW480 | 5.8 |
| carcinoma SCC-4 | | met) SW620 | |
| Testis Pool | 10.0 | Colon ca. HT29 | 1.0 |
| Prostate ca.* (bone | 19.9 | Colon ca. HCT-116 | 5.8 |
| met) PC-3 | | | |
| Prostate Pool | 12.9 | Colon ca. CaCo-2 | 8.2 |
| Placenta | 1.9 | Colon cancer tissue | 7.6 |
| Uterus Pool | 9.2 | Colon ca. SW1116 | 2.0 |
| Ovarian ca. | 6.6 | Colon ca. Colo-205 | 9.0 |
| OVCAR-3 | | | |
| Ovarian ca. SK- | 12.2 | Colon ca. SW-48 | 1.7 |
| OV-3 | | | |
| Ovarian ca. | 1.9 | Colon Pool | 20.7 |
| OVCAR-4 | | | |
| Ovarian ca. | 8.5 | Small Intestine Pool | 27.9 |
| OVCAR-5 | | | |
| Ovarian ca. | 1.2 | Stomach Pool | 28.7 |
| IGROV-1 | | | |
| Ovarian ca. | 1.7 | Bone Marrow Pool | 8.0 |
| OVCAR-8 | | | |
| Ovary | 12.1 | Fetal Heart | 30.6 |
| Breast ca. MCF-7 | 63.7 | Heart Pool | 16.7 |
| Breast ca. MDA- | 4.5 | Lymph Node Pool | 18.8 |
| MB-231 | | | |
| Breast ca. BT 549 | 1.8 | Fetal Skeletal Muscle | 5.4 |
| Breast ca. T47D | 10.8 | Skeletal Muscle Pool | 66.0 |
| Breast ca. MDA-N | 2.3 | Spleen Pool | 14.9 |
| Breast Pool | 39.0 | Thymus Pool | 16.4 |
| Trachea | 15.3 | CNS cancer | 5.8 |
| | | (glio/astro) U87-MG | |
| Lung | 5.6 | CNS cancer | 9.1 |
| | | (glio/astro) U-118-MG | |
| Fetal Lung | 13.6 | CNS cancer | 5.0 |
| | | (neuro; met) SK-N-AS | |
| Lung ca. NCI-N417 | 2.3 | CNS cancer (astro) | 3.1 |
| | | SF-539 | |
| Lung ca. LX-1 | 6.4 | CNS cancer (astro) | 3.7 |
| | | SNB-75 | |
| Lung ca. NCI-H146 | 2.2 | CNS cancer (glio) | 1.3 |
| | | SNB-19 | |
| Lung ca. SHP-77 | 2.8 | CNS cancer (glio) | 8.1 |
| | | SF-295 | |
| Lung ca. A549 | 10.0 | Brain (Amygdala) | 6.2 |
| | | Pool | |
| Lung ca. NCI-H526 | 0.9 | Brain (cerebellum) | 13.0 |
| Lung ca. NCI-H23 | 26.6 | Brain (fetal) | 4.2 |
| Lung ca. NCI-H460 | 3.9 | Brain (Hippocampus) | 7.5 |
| | | Pool | |
| Lung ca. HOP-62 | 1.4 | Cerebral Cortex Pool | 8.4 |
| Lung ca. NCI-H522 | 13.5 | Brain (Substantia | 7.0 |
| | | nigra) Pool | |
| Liver | 23.2 | Brain (Thalamus) Pool | 10.2 |
| Fetal Liver | 11.7 | Brain (whole) | 8.8 |
| Liver ca. HepG2 | 6.6 | Spinal Cord Pool | 9.1 |
| Kidney Pool | 38.7 | Adrenal Gland | 50.0 |
| Fetal Kidney | 10.4 | Pituitary gland Pool | 7.4 |
| Renal ca. 786-0 | 1.5 | Salivary Gland | 11.3 |
| Renal ca. A498 | 1.4 | Thyroid (female) | 5.1 |
| Renal ca. ACHN | 3.0 | Pancreatic ca. | 1.7 |
| | | CAPAN2 | |
| Renal ca. UO-31 | 2.5 | Pancreas Pool | 17.0 |

General_screening_panel_v1.4 Summary: Ag3502 The CG59641-01 encodes an acetyl-CoA carboxylase 2 (ACC2) protein. Expression of this gene is highest in adipose tissue (CT=25.5). High levels of expression of this gene are also detected in other tissues with metabolic or endocrine function such as pancreas, adrenal gland, gastrointestinal tract, heart, skeletal muscle, and thyroid. Acetyl-coenzyme A (acetyl-CoA) carboxylase (ACC) catalyzes the synthesis of malonyl-CoA, a metabolite that plays a pivotal role in the synthesis and oxidation of fatty. Hence, ACC links fatty acid and carbohydrate metabolism through the shared intermediate acetyl-CoA, the product of pyruvate dehydrogenase. It has been shown recently that mutations in ACC2 gene lead to loss of body fat in a normal caloric intake in mouse (Abu-Elheiga et al., 2001). Therefore, therapeutic modulation of the activity of this gene may prove useful in the treatment of endocrine/metabolically related diseases, such as obesity and diabetes.

Low to moderate expression of this gene is also detected in most of the samples used in this panel suggesting the possibility of a wider role in intercellular signaling for this molecule.

Among tissues that originate in the central nervous system, this gene is expressed in all regions represented on this panel. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, significantly higher levels of expression are seen in a breast cancer cell line. Thus, expression of this gene could be used to differentiate between this sample and other samples on this panel and as a marker to detect the presence of breast cancer. Furthermore, therapeutic modulation of the expression or function of this gene may be effective in the treatment of breast cancer.

### REFERENCES

1. Abu-Elheiga L, Matzuk M M, Abo-Hashema K A, Wakil S J. (2001) Continuous fatty acid oxidation and reduced fat storage in mice lacking acetyl-CoA carboxylase 2. Science Mar. 30, 2001;291(5513):2613-6

BT. CG59630-01: Midnolin

Expression of gene CG59630-01 was assessed using the primer-probe set Ag3425, described in Table BTA. Results of the RTQ-PCR runs are shown in Tables BTB, BTC and BTD.

TABLE BTA

Probe Name Ag3425

| | | | | SEQ ID |
|---|---|---|---|---|
| Primers | Sequences | Length | Start Position | NO: |
| Forward | 5′-aagctgaccttggtacccac-3′ | 20 | 295 | 617 |
| Probe | TET-5′-ctcatgtctcaggcctcaaggcc-3′-TAMRA | 23 | 328 | 618 |
| Reverse | 5′-ctctcgagagcttgcatcac-3′ | 20 | 361 | 619 |

TABLE BTB
CNS_neurodegeneration_v1.0

| | Rel. Exp. | | Rel. Exp. |
|---|---|---|---|
| | (%) Ag3425, | | (%) Ag3425, |
| | Run | | Run |
| Tissue Name | 210350911 | Tissue Name | 210350911 |
| AD 1 Hippo | 18.6 | Control (Path) 3 | 14.0 |
| | | Temporal Ctx | |
| AD 2 Hippo | 44.1 | Control (Path) 4 | 25.0 |
| | | Temporal Ctx | |
| AD 3 Hippo | 11.3 | AD 1 Occipital Ctx | 13.6 |
| AD 4 Hippo | 19.3 | AD 2 Occipital Ctx | 0.0 |
| | | (Missing) | |
| AD 5 Hippo | 53.2 | AD 3 Occipital Ctx | 11.5 |
| AD 6 Hippo | 100.0 | AD 4 Occipital Ctx | 24.7 |
| Control 2 Hippo | 47.0 | AD 5 Occipital Ctx | 53.2 |
| Control 4 Hippo | 38.2 | AD 6 Occipital Ctx | 80.7 |
| Control (Path) 3 | 10.5 | Control 1 Occipital | 19.9 |
| Hippo | | Ctx | |
| AD 1 Temporal | 17.3 | Control 2 Occipital | 49.3 |
| Ctx | | Ctx | |
| AD 2 Temporal | 33.7 | Control 3 Occipital | 31.2 |
| Ctx | | Ctx | |
| AD 3 Temporal | 10.4 | Control 4 Occipital | 13.9 |
| Ctx | | Ctx | |
| AD 4 Temporal | 19.1 | Control (Path) 1 | 81.2 |
| Ctx | | Occipital Ctx | |
| AD 5 Inf Temporal | 49.3 | Control (Path) 2 | 11.0 |
| Ctx | | Occipital Ctx | |
| AD 5 Sup | 45.4 | Control (Path) 3 | 8.4 |
| Temporal Ctx | | Occipital Ctx | |
| AD 6 Inf Temporal | 97.3 | Control (Path) 4 | 19.6 |
| Ctx | | Occipital Ctx | |
| AD 6 Sup | 85.9 | Control 1 Parietal | 18.8 |
| Temporal Ctx | | Ctx | |
| Control 1 | 20.3 | Control 2 Parietal | 29.9 |
| Temporal Ctx | | Ctx | |
| Control 2 | 59.9 | Control 3 Parietal | 32.3 |
| Temporal Ctx | | Ctx | |
| Control 3 | 32.3 | Control (Path) 1 | 91.4 |
| Temporal Ctx | | Parietal Ctx | |
| Control 3 | 17.4 | Control (Path) 2 | 32.3 |
| Temporal Ctx | | Parietal Ctx | |
| Control (Path) 1 | 70.7 | Control (Path) 3 | 7.7 |
| Temporal Ctx | | Parietal Ctx | |
| Control (Path) 2 | 27.5 | Control (Path) 4 | 45.1 |
| Temporal Ctx | | Parietal Ctx | |

TABLE BTC
General_screening_panel_v1.4

| | Rel. Exp. | | Rel. Exp. |
|---|---|---|---|
| | (%) Ag3425, | | (%) Ag3425, |
| | Run | | Run |
| Tissue Name | 217049295 | Tissue Name | 217049295 |
| Adipose | 17.8 | Renal ca. TK-10 | 26.4 |
| Melanoma* | 15.5 | Bladder | 16.0 |
| Hs688(A).T | | | |
| Melanoma* | 19.5 | Gastric ca. (liver met.) | 26.6 |
| Hs688(B).T | | NCI-N87 | |
| Melanoma* M14 | 10.5 | Gastric ca. KATO III | 23.0 |
| Melanoma* | 15.7 | Colon ca. SW-948 | 11.6 |
| LOXIMVI | | | |
| Melanoma* SK- | 8.0 | Colon ca. SW480 | 27.5 |
| MEL-5 | | | |
| Squamous cell | 27.2 | Colon ca.* (SW480 | 15.3 |
| carcinoma SCC-4 | | met) SW620 | |
| Testis Pool | 5.6 | Colon ca. HT29 | 15.2 |
| Prostate ca.* (bone | 18.7 | Colon ca. HCT-116 | 40.6 |
| met) PC-3 | | | |
| Prostate Pool | 3.3 | Colon ca. CaCo-2 | 41.8 |
| Placenta | 16.8 | Colon cancer tissue | 19.6 |
| Uterus Pool | 2.6 | Colon ca. SW1116 | 9.5 |
| Ovarian ca. | 27.7 | Colon ca. Colo-205 | 41.5 |
| OVCAR-3 | | | |
| Ovarian ca. SK- | 58.2 | Colon ca. SW-48 | 6.8 |
| OV-3 | | | |
| Ovarian ca. | 4.0 | Colon Pool | 5.9 |
| OVCAR-4 | | | |
| Ovarian ca. | 27.2 | Small Intestine Pool | 6.6 |
| OVCAR-5 | | | |
| Ovarian ca. | 30.1 | Stomach Pool | 42.9 |
| IGROV-1 | | | |
| Ovarian ca. | 19.3 | Bone Marrow Pool | 2.7 |
| OVCAR-8 | | | |
| Ovary | 7.6 | Fetal Heart | 9.0 |
| Breast ca. MCF-7 | 37.1 | Heart Pool | 5.7 |
| Breast ca. MDA- | 15.3 | Lymph Node Pool | 6.5 |
| MB-231 | | | |
| Breast ca. BT 549 | 51.1 | Fetal Skeletal Muscle | 6.6 |
| Breast ca. T47D | 100.0 | Skeletal Muscle Pool | 17.4 |
| Breast ca. MDA-N | 8.3 | Spleen Pool | 11.2 |
| Breast Pool | 41.8 | Thymus Pool | 8.0 |
| Trachea | 15.9 | CNS cancer | 46.3 |
| | | (glio/astro) U87-MG | |
| Lung | 2.6 | CNS cancer | 20.7 |
| | | (glio/astro) U-118-MG | |
| Fetal Lung | 55.5 | CNS cancer | 29.1 |
| | | (neuro; met) SK-N-AS | |
| Lung ca. NCI-N417 | 43.5 | CNS cancer (astro) | 13.8 |
| | | SF-539 | |
| Lung ca. LX-1 | 23.5 | CNS cancer (astro) | 36.9 |
| | | SNB-75 | |
| Lung ca. NCI-H146 | 5.8 | CNS cancer (glio) | 29.1 |
| | | SNB-19 | |
| Lung ca. SHP-77 | 11.7 | CNS cancer (glio) | 73.7 |
| | | SF-295 | |
| Lung ca. A549 | 21.5 | Brain (Amygdala) | 5.3 |
| | | Pool | |
| Lung ca. NCI-H526 | 15.7 | Brain (cerebellum) | 14.6 |
| Lung ca. NCI-H23 | 12.9 | Brain (fetal) | 20.6 |
| Lung ca. NCI-H460 | 49.3 | Brain (Hippocampus) | 4.6 |
| | | Pool | |
| Lung ca. HOP-62 | 10.1 | Cerebral Cortex Pool | 4.9 |
| Lung ca. NCI-H522 | 16.2 | Brain (Substantia | 10.4 |
| | | nigra) Pool | |
| Liver | 1.3 | Brain (Thalamus) Pool | 6.1 |
| Fetal Liver | 9.3 | Brain (whole) | 45.1 |
| Liver ca. HepG2 | 29.7 | Spinal Cord Pool | 5.8 |
| Kidney Pool | 8.0 | Adrenal Gland | 13.6 |
| Fetal Kidney | 11.3 | Pituitary gland Pool | 6.8 |
| Renal ca. 786-0 | 23.0 | Salivary Gland | 4.6 |
| Renal ca. A498 | 18.2 | Thyroid (female) | 13.9 |
| Renal ca. ACHN | 13.6 | Pancreatic ca. | 7.6 |
| | | CAPAN2 | |
| Renal ca. UO-31 | 28.5 | Pancreas Pool | 14.0 |

TABLE BTD
Panel 4.1D

| | Rel. Exp. (%) | | Rel. Exp. (%) |
|---|---|---|---|
| | Ag3425, Run | | Ag3425, Run |
| Tissue Name | 169839020 | Tissue Name | 169839020 |
| Secondary Th1 act | 20.6 | HUVEC IL-1beta | 46.7 |
| Secondary Th2 act | 30.6 | HUVEC IFN gamma | 23.3 |
| Secondary Tr1 act | 32.1 | HUVEC TNF alpha + | 13.3 |
| | | IFN gamma | |
| Secondary Th1 rest | 18.2 | HUVEC TNF alpha + | 22.8 |
| | | IL4 | |
| Secondary Th2 rest | 23.8 | HUVEC IL-11 | 22.5 |
| Secondary Tr1 rest | 12.6 | Lung Microvascular EC | 37.9 |
| | | none | |
| Primary Th1 act | 2.8 | Lung Microvascular EC | 22.2 |
| | | TNF alpha + IL-1beta | |
| Primary Th2 act | 24.7 | Microvascular Dermal | 24.1 |
| | | EC none | |
| Primary Tr1 act | 20.0 | Microsvasular Dermal | 21.2 |
| | | EC TNF alpha + IL-1beta | |
| Primary Th1 rest | 18.6 | Bronchial epithelium | 26.6 |
| | | TNF alpha + IL-1beta | |
| Primary Th2 rest | 19.5 | Small airway epithelium | 23.5 |
| | | none | |
| Primary Tr1 rest | 22.4 | Small airway epithelium | 27.2 |
| | | TNF alpha + IL-1beta | |
| CD45RA CD4 | 17.7 | Coronery artery SMC rest | 22.1 |
| lymphocyte act | | | |
| CD45RO CD4 | 19.8 | Coronery artery SMC | 20.2 |
| lymphocyte act | | TNF alpha + IL-1beta | |
| CD8 lymphocyte act | 14.1 | Astrocytes rest | 24.7 |
| Secondary CD8 | 23.8 | Astrocytes TNF alpha + | 17.8 |
| lymphocyte rest | | IL-1beta | |
| Secondary CD8 | 12.2 | KU-812 (Basophil) rest | 16.5 |
| lymphocyte act | | | |
| CD4 lymphocyte none | 6.5 | KU-812 (Basophil) | 36.6 |
| | | PMA/ionomycin | |
| 2ry Th1/Th2/Tr1_anti- | 15.2 | CCD1106 | 40.1 |
| CD95 CH11 | | (Keratinocytes) none | |
| LAK cells rest | 34.2 | CCD1106 | 41.8 |
| | | (Keratinocytes) | |
| | | TNF alpha + IL-1beta | |
| LAK cells IL-2 | 24.5 | Liver cirrhosis | 20.0 |
| LAK cells IL-2 + IL-12 | 21.6 | NCI-H292 none | 41.2 |
| LAK cells IL-2 + IFN | 23.2 | NCI-H292 IL-4 | 58.6 |
| gamma | | | |
| LAK cells IL-2 + IL-18 | 22.2 | NCI-H292 IL-9 | 61.6 |
| LAK cells | 97.3 | NCI-H292 IL-13 | 47.6 |
| PMA/ionomycin | | | |
| NK Cells IL-2 rest | 28.7 | NCI-H292 IFN gamma | 54.0 |
| Two Way MLR 3 day | 32.5 | HPAEC none | 16.6 |
| Two Way MLR 5 day | 30.1 | HPAEC TNF alpha + IL- | 20.7 |
| | | 1beta | |
| Two Way MLR 7 day | 18.7 | Lung fibroblast none | 46.0 |
| PBMC rest | 27.9 | Lung fibroblast TNF | 20.2 |
| | | alpha + IL-1beta | |
| PBMC PWM | 25.3 | Lung fibroblast IL-4 | 47.0 |
| PBMC PHA-L | 22.7 | Lung fibroblast IL-9 | 52.1 |
| Ramos (B cell) none | 25.5 | Lung fibroblast IL-13 | 45.1 |
| Ramos (B cell) | 23.5 | Lung fibroblast IFN | 50.0 |
| ionomycin | | gamma | |
| B lymphocytes PWM | 17.8 | Dermal fibroblast | 28.3 |
| | | CCD1070 rest | |
| B lymphocytes CD40L | 23.8 | Dermal fibroblast | 37.1 |
| and IL-4 | | CCD1070 TNF alpha | |
| EOL-1 dbcAMP | 34.4 | Dermal fibroblast | 13.3 |
| | | CCD1070 IL-1beta | |
| EOL-1 dbcAMP | 87.7 | Dermal fibroblast IFN | 28.5 |
| PMA/ionomycin | | gamma | |
| Dendritic cells none | 32.3 | Dermal fibroblast IL-4 | 26.2 |
| Dendritic cells LPS | 21.5 | Dermal Fibroblasts rest | 26.6 |
| Dendritic cells anti- | 32.8 | Neutrophils TNFa + LPS | 33.7 |
| CD40 | | | |
| Monocytes rest | 54.3 | Neutrophils rest | 100.0 |
| Monocytes LPS | 93.3 | Colon | 18.4 |
| Macrophages rest | 23.8 | Lung | 28.7 |
| Macrophages LPS | 36.1 | Thymus | 28.1 |
| HUVEC none | 24.7 | Kidney | 14.0 |
| HUVEC starved | 38.4 | | |

CNS_neurodegeneration_v1.0 Summary: Ag3425 This panel confirms the expression of this gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3425 The CG59630-01 gene is a homologue of mouse midnoline (midbrain nucleolar protein). Its expression is moderate to high across all of the samples on this panel, with highest expression in a breast cancer cell line (CT=25.3). The widespread expression suggests that this gene may play an important role in cellular function. In mouse, the expression of this gene is developmentally regulated: it is strongly expressed at the mesencephalon (midbrain) of the embryo and is involved in regulation of genes related to neurogenesis in the nucleolus (Tsukahara et al., 2000). Based on the gene's expression in all CNS regions examined, this gene may therefore play a role in central nervous system disorders such as Alzheimer's disease, Parkinson's disease, epilepsy, multiple sclerosis, schizophrenia and depression.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

### REFERENCE

1. Tsukahara M, Suemori H, Noguchi S, Ji Z S, Tsunoo H. (2000) Novel nucleolar protein, midnolin, is expressed in the mesencephalon during mouse development. Gene Aug. 22, 2000;254(1-2):45-55

Panel 4.1D Summary: Ag3425 The CG59630-01 gene is a homologue of mouse midnoline (midbrain nucleolar protein). Its expression is moderate to high across all of the samples on this panel, with highest expression in resting neutrophils (CT=29.1). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

BU. CG59561-01: Cytosolic Acyl Coenzyme a Thioester Hydrolase

Expression of gene CG59561-01 was assessed using the primer-probe set Ag3424, described in Table BUA. Results of the RTQ-PCR runs are shown in Tables BUB, BUC and BUD.

TABLE BUA

Probe Name Ag3424

| | | | Start | SEQ ID |
|---|---|---|---|---|
| Primers | Sequences | Length | Position | NO: |
| Forward | 5′-aagctgaccaataaggccac-3′ | 20 | 345 | 620 |
| Probe | TET-5′-gtggacaaggtcctcgaagagcctc-3′-TAMRA | 25 | 396 | 621 |
| Reverse | 5′-ctgccggaaatacacaacag-3′ | 20 | 421 | 622 |

TABLE BUB
CNS_neurodegeneration_v1.0

| | Rel. Exp. | | Rel. Exp. (%) |
|---|---|---|---|
| | (%) Ag3424 | | (%) Ag3424 |
| | Run | | Run |
| Tissue Name | 210350585 | Tissue Name | 210350585 |
| AD 1 Hippo | 8.8 | Control (Path) 3 | 1.5 |
| | | Temporal Ctx | |
| AD 2 Hippo | 16.7 | Control (Path) 4 | 25.5 |
| | | Temporal Ctx | |
| AD 3 Hippo | 2.7 | AD 1 Occipital | 3.7 |
| | | Ctx | |
| AD 4 Hippo | 4.7 | AD 2 Occipital | 0.0 |
| | | Ctx (Missing) | |
| AD 5 hippo | 100.0 | AD 3 Occipital | 2.0 |
| | | Ctx | |
| AD 6 Hippo | 34.6 | AD 4 Occipital | 8.5 |
| | | Ctx | |
| Control 2 Hippo | 44.8 | AD 5 Occipital | 11.7 |
| | | Ctx | |
| Control 4 Hippo | 2.2 | AD 6 Occipital | 90.8 |
| | | Ctx | |
| Control (Path) 3 | 2.0 | Control 1 Occipital | 0.8 |
| Hippo | | Ctx | |
| AD 1 Temporal Ctx | 2.4 | Control 2 Occipital | 84.1 |
| | | Ctx | |
| AD 2 Temporal Ctx | 16.6 | Control 3 Occipital | 5.1 |
| | | Ctx | |
| AD 3 Temporal Ctx | 2.1 | Control 4 Occipital | 1.3 |
| | | Ctx | |
| AD 4 Temporal Ctx | 8.6 | Control (Path) 1 | 96.6 |
| | | Occipital Ctx | |
| AD 5 Inf Temporal | 62.9 | Control (Path) 2 | 5.7 |
| Ctx | | Occipital Ctx | |
| AD 5 SupTemporal | 28.3 | Control (Path) 3 | 0.5 |
| Ctx | | Occipital Ctx | |
| AD 6 Inf Temporal | 26.6 | Control (Path) 4 | 8.8 |
| Ctx | | Occipital Ctx | |
| AD 6 Sup Temporal | 22.1 | Control 1 Parietal | 2.0 |
| Ctx | | Ctx | |
| Control 1 Temporal | 1.2 | Control 2 Parietal | 22.1 |
| Ctx | | Ctx | |
| Control 2 Temporal | 65.1 | Control 3 Parietal | 19.9 |
| Ctx | | Ctx | |
| Control 3 Temporal | 8.7 | Control (Path) 1 | 94.0 |
| Ctx | | Parietal Ctx | |
| Control 4 Temporal | 2.4 | Control (Path) 2 | 14.0 |
| Ctx | | Parietal Ctx | |
| Control (Path) 1 | 61.1 | Control (Path) 3 | 0.9 |
| Temporal Ctx | | Parietal Ctx | |
| Control (Path) 2 | 28.3 | Control (Path) 4 | 39.0 |
| Temporal Ctx | | Parietal Ctx | |

TABLE BUC
Panel 4D

| | Rel. Exp. (%) | | Rel. Exp. (%) |
|---|---|---|---|
| | Ag3424, Run | | Ag3424, Run |
| Tissue Name | 166385382 | Tissue Name | 166385382 |
| Secondary Th1 act | 62.9 | HUVEC IL-1beta | 4.0 |
| Secondary Th2 act | 37.9 | HUVEC IFN gamma | 10.7 |
| Secondary Tr1 act | 68.8 | HUVEC TNF alpha + | 18.6 |
| | | IFN gamma | |
| Secondary Th1 rest | 4.2 | HUVEC TNF alpha + | 15.5 |
| | | IL4 | |
| Secondary Th2 rest | 12.1 | HUVEC IL-11 | 10.2 |
| Secondary Tr1 rest | 4.4 | Lung Microvascular EC | 28.7 |
| | | none | |
| Primary Th1 act | 66.9 | Lung Microvascular EC | 25.7 |
| | | TNF alpha + IL-1beta | |
| Primary Th2 act | 61.1 | Microvascular Dermal | 36.3 |
| | | EC none | |
| Primary Tr1 act | 43.2 | Microsvasular Dermal | 20.3 |
| | | EC TNF alpha + IL-1beta | |
| Primary Th1 rest | 30.1 | Bronchial epithelium | 25.5 |
| | | TNF alpha + IL-1beta | |
| Primary Th2 rest | 17.4 | Small airway epithelium | 19.5 |
| | | none | |
| Primary Tr1 rest | 15.9 | Small airway epithelium | 52.1 |
| | | TNF alpha + IL-1beta | |
| CD45RA CD4 | 14.8 | Coronery artery SMC rest | 14.8 |
| lymphocyte act | | | |
| CD45RO CD4 | 37.6 | Coronery artery SMC | 8.3 |
| lymphocyte act | | TNF alpha + IL-1beta | |
| CD8 lymphocyte act | 43.8 | Astrocytes rest | 13.9 |
| Secondary CD8 | 49.7 | Astrocytes TNF alpha + | 13.3 |
| lymphocyte rest | | IL-1beta | |
| Secondary CD8 | 24.1 | KU-812 (Basophil) rest | 23.8 |
| lymphocyte act | | | |
| CD4 lymphocyte none | 0.9 | KU-812 (Basophil) | 51.4 |
| | | PMA/ionomycin | |
| 2ry Th1/Th2/Tr1_anti- | 9.0 | CCD1106 | 62.4 |
| CD95 CH11 | | (Keratinocytes) none | |
| LAK cells rest | 33.0 | CCD1106 | 37.1 |
| | | (Keratinocytes) | |
| | | TNF alpha + IL-1beta | |
| LAK cells IL-2 | 27.7 | Liver cirrhosis | 3.5 |
| LAK cells IL-2 + IL-12 | 25.5 | Lupus kidney | 1.0 |
| LAK cells IL-2 + IFN | 35.6 | NCI-H292 none | 16.2 |
| gamma | | | |
| LAK cells IL-2 + IL-18 | 21.9 | NCI-H292 IL-4 | 23.5 |
| LAK cells | 3.6 | NCI-H292 IL-9 | 29.5 |
| PMA/ionomycin | | | |
| NK Cells IL-2 rest | 15.1 | NCI-H292 IL-13 | 13.4 |
| Two Way MLR 3 day | 9.4 | NCI-H292 IFN gamma | 20.6 |
| Two Way MLR 5 day | 17.4 | HPAEC none | 17.4 |
| Two Way MLR 7 day | 13.1 | HPAEC TNF alpha + IL- | 20.9 |
| | | 1beta | |
| PBMC rest | 0.8 | Lung fibroblast none | 37.9 |
| PBMC PWM | 88.9 | Lung fibroblast TNF | 34.9 |
| | | alpha + IL-1beta | |
| PBMC PHA-L | 52.5 | Lung fibroblast IL-4 | 69.7 |
| Ramos (B cell) none | 17.2 | Lung fibroblast IL-9 | 49.7 |
| Ramos (B cell) | 31.9 | Lung fibroblast IL-13 | 45.4 |
| ionomycin | | | |
| B lymphocytes PWM | 75.8 | Lung fibroblast IFN | 90.8 |
| | | gamma | |
| B lymphocytes CD40L | 9.9 | Dermal fibroblast | 54.3 |
| and IL-4 | | CCD1070 rest | |
| EOL-1 dbcAMP | 11.0 | Dermal fibroblast | 84.1 |
| | | CCD1070 TNF alpha | |
| EOL-1 dbcAMP | 8.1 | Dermal fibroblast | 30.6 |
| PMA/ionomycin | | CCD1070 IL-1beta | |
| Dendritic cells none | 38.2 | Dermal fibroblast IFN | 31.4 |
| | | gamma | |
| Dendritic cells LPS | 31.9 | Dermal fibroblast IL-4 | 45.4 |
| Dendritic cells anti- | 37.1 | IBD Colitis 2 | 2.5 |
| CD40 | | | |
| Monocytes rest | 0.4 | IBD Crohn's | 4.7 |
| Monocytes LPS | 0.6 | Colon | 45.4 |
| Macrophages rest | 12.6 | Lung | 16.6 |
| Macrophages LPS | 5.8 | Thymus | 100.0 |
| HUVEC none | 16.0 | Kidney | 20.6 |
| HUVEC starved | 27.4 | | |

TABLE BUD
Panel 5 Islet

| | Rel. Exp. (%) | | Rel. Exp. (%) |
|---|---|---|---|
| | Ag3424, Run | | Ag3424, Run |
| Tissue Name | 242385366 | Tissue Name | 242385366 |
| 97457_Patient- | 8.2 | 94709_Donor 2 AM - A_adipose | 6.7 |
| 02go_adipose | | | |
| 97476_Patient- | 3.9 | 94710_Donor 2 AM - B_adipose | 8.7 |
| 07sk_skeletal muscle | | | |
| 97477_Patient- | 7.4 | 94711_Donor 2 AM - C_adipose | 5.9 |
| 07ut_uterus | | | |
| 97478_Patient- | 7.9 | 94712_Donor 2 AD - A - | 8.3 |
| 07pl_placenta | | adipose | |
| 99167_Bayer Patient | 4.8 | 94713_Donor 2 AD - B_adipose | 5.4 |
| 1 | | | |
| 97482_Patient- | 4.1 | 94714_Donor 2 AD - C_adipose | 7.7 |
| 08ut_uterus | | | |
| 97483_Patient- | 2.4 | 94742_Donor 3 U - | 3.0 |
| 08pl_placenta | | A_Mesenchymal Stem Cells | |
| 97486_Patient- | 0.0 | 94743_Donor 3 U - | 9.9 |
| 09sk_skeletal muscle | | B_Mesenchymal Stem Cells | |
| 97487_Patient- | 5.3 | 94730_Donor 3 AM - A_adipose | 22.5 |
| 09ut_uterus | | | |
| 97488_Patient- | 2.3 | 94731_Donor 3 AM - B_adipose | 15.1 |
| 09pl_placenta | | | |
| 97492_Patient- | 4.8 | 94732_Donor 3 AM - C_adipose | 10.2 |
| 10ut_uterus | | | |
| 97493_Patient- | 6.6 | 94733_Donor 3 AD - A_adipose | 29.7 |
| 10pl_placenta | | | |
| 97495_Patient- | 0.9 | 94734_Donor 3 AD - B_adipose | 6.4 |
| 11go_adipose | | | |
| 97496_Patient- | 0.3 | 94735_Donor 3 AD - C_adipose | 34.9 |
| 11sk_skeletal muscle | | | |
| 97497_Patient- | 9.8 | 77138_Liver_HepG2untreated | 57.8 |
| 11ut_uterus | | | |
| 97498_Patient- | 3.0 | 73556_Heart_Cardiac stromal | 10.3 |
| 11pl_placenta | | cells (primary) | |
| 97500_Patient- | 1.7 | 81735_Small Intestine | 23.7 |
| 12go_adipose | | | |
| 97501_Patient- | 1.4 | 72409_Kidney_Proximal | 4.5 |
| 12sk_skeletal muscle | | Convoluted Tubule | |
| 97502_Patient- | 10.2 | 82685_Small | 1.2 |
| 12ut_uterus | | intestine_Duodenum | |
| 97503_Patient- | 3.2 | 90650_Adrenal_Adrenocortical | 1.4 |
| 12pl_placenta | | adenoma | |
| 94721_Donor 2 U - | 6.8 | 72410_Kidney_HRCE | 100.0 |
| A_Mesenchymal | | | |
| Stem Cells | | | |
| 94722_Donor 2 U - | 2.8 | 72411_Kidney_HRE | 69.7 |
| B_Mesenchymal | | | |
| Stem Cells | | | |
| 94723_Donor 2 U - | 5.1 | 73139_Uterus_Uterine smooth | 22.4 |
| C_Mesenchymal | | muscle cells | |
| Stem Cells | | | |

CNS_neurodegeneration_v1.0 Summary: Ag3424 This panel confirms the expression of the CG59561-01 gene at low levels in the brain in an independent group of individuals. However, no differential expression of this gene was detected between Alzheimer's diseased postmortem brains and those of non-demented controls in this experiment. This expression profile suggests that this gene may play a role in central nervous system disorders such as Alzheimer's disease, Parkinson's disease, epilepsy, multiple sclerosis, schizophrenia and depression.

General_screening_panel_v1.4 Summary: Ag3424 Results from one experiment with the CG59561-01 gene are not included. The amp plot indicates that there were experimental difficulties with this run. (Data not shown.)

Panel 4D Summary: Ag3424 The CG59561-01 gene encodes a protein homologous to cytosolic acyl coenzyme A thioester hydrolase (Brain acyl-CoA hydrolase, BACH). Among the tissue samples used in this panel, highest expression of this gene is detected in thymus (CT=29.6). In addition, expression of this gene is stimulated in activated primary and secondary—Th1, Th2 and Tr1 cells. Therefore, this gene product may play an important role in T cell development. Thus, therapeutics designed with the protein encoded for by this transcript could be important in regulating T cell function and treating T cell mediated diseases such as emphysema, asthma, arthritis, psoriasis, IBD, and systemic lupus erythematosus.

Interestingly, expression of this gene is also seen in activated PBMCs (CTs=30) as compared to resting PBMCs (CT=36) suggesting a role for this gene product in B-cell and T-cell proliferation. Therefore, small molecules that antagonize the function of this gene product may be useful as therapeutic drugs to reduce or eliminate the symptoms in patients with autoimmune and inflammatory diseases in which B cells play a part in the initiation or progression of the disease process, such as systemic lupus erythematosus, Crohn's disease, ulcerative colitis, multiple sclerosis, chronic obstructive pulmonary disease, asthma, emphysema, rheumatoid arthritis, or psoriasis.

Panel 5 Islet Summary: Ag3424 The CG59561-01 gene is expressed at low levels in adipose and placenta, with highest expression in the kidney (CT=30.8). As an enzyme involved in lipid homeostasis, therapeutic modulation of this gene product may be a treatment for obesity and obesity-related diseases, including Type 2 diabetes.

BV. CG59452-01: Cell Proliferation Related Protein Cap

Expression of gene CG59452-01 was assessed using the primer-probe set Ag3443, described in Table BVA. Results of the RTQ-PCR runs are shown in Tables BVB and BVC.

TABLE BVA

Probe Name Ag3443

| | | | Start | SEQ ID |
|---|---|---|---|---|
| Primers | Sequences | Length | Position | NO: |
| Forward | 5′-caggaatgtatccaggacttca-3′ | 22 | 387 | 623 |
| Probe | TET-5′-catctacaacaagcctggagatgaca-3′-TAMRA | 26 | 431 | 624 |
| Reverse | 5′-tttccagagcttctgccatt-3′ | 20 | 464 | 625 |

TABLE BVB
CNS_neurodegeneration_v1.0

| | Rel. Exp. | | Rel. Exp. |
|---|---|---|---|
| | (%) Ag3443, | | (%) Ag3443, |
| | Run | | Run |
| Tissue Name | 210374885 | Tissue Name | 210374885 |
| AD 1 Hippo | 10.2 | Control (Path) 3 | 6.2 |
| | | Temporal Ctx | |
| AD 2 Hippo | 28.3 | Control (Path) 4 | 27.0 |
| | | Temporal Ctx | |
| AD 3 Hippo | 8.0 | AD 1 Occipital Ctx | 13.6 |
| AD 4 Hippo | 4.8 | AD 2 Occipital Ctx | 0.0 |
| | | (Missing) | |
| AD 5 Hippo | 84.1 | AD 3 Occipital Ctx | 6.0 |
| AD 6 Hippo | 71.7 | AD 4 Occipital Ctx | 17.9 |
| Control 2 Hippo | 31.4 | AD 5 Occipital Ctx | 54.3 |
| Control 4 Hippo | 6.1 | AD 6 Occipital Ctx | 22.7 |
| Control (Path) 3 | 3.3 | Control 1 Occipital | 4.0 |
| Hippo | | Ctx | |
| AD 1 Temporal | 21.9 | Control 2 Occipital | 73.7 |
| Ctx | | Ctx | |
| AD 2 Temporal | 33.4 | Control 3 Occipital | 13.7 |
| Ctx | | Ctx | |
| AD 3 Temporal | 7.4 | Control 4 Occipital | 7.7 |
| Ctx | | Ctx | |
| AD 4 Temporal | 16.8 | Control (Path) 1 | 100.0 |
| Ctx | | Occipital Ctx | |
| AD 5 Inf Temporal | 74.7 | Control (Path) 2 | 11.0 |
| Ctx | | Occipital Ctx | |
| AD 5 Sup | 36.1 | Control (Path) 3 | 1.3 |
| Temporal Ctx | | Occipital Ctx | |
| AD 6 Inf Temporal | 79.0 | Control (Path) 4 | 15.3 |
| Ctx | | Occipital Ctx | |
| AD 6 Sup | 85.9 | Control 1 Parietal | 6.1 |
| Temporal Ctx | | Ctx | |
| Control 1 | 8.6 | Control 2 Parietal | 35.8 |
| Temporal Ctx | | Ctx | |
| Control 2 | 48.3 | Control 3 Parietal | 12.9 |
| Temporal Ctx | | Ctx | |
| Control 3 | 11.4 | Control (Path) 1 | 59.5 |
| Temporal Ctx | | Parietal Ctx | |
| Control 3 | 6.5 | Control (Path) 2 | 25.5 |
| Temporal Ctx | | Parietal Ctx | |
| Control (Path) 1 | 93.3 | Control (Path) 3 | 2.4 |
| Temporal Ctx | | Parietal Ctx | |
| Control (Path) 2 | 23.8 | Control (Path) 4 | 23.2 |
| Temporal Ctx | | Parietal Ctx | |

TABLE BVC
Panel 4D

| | Rel. Exp. (%) | | Rel. Exp. (%) |
|---|---|---|---|
| | Ag3443, Run | | Ag3443, Run |
| Tissue Name | 166397102 | Tissue Name | 166397102 |
| Secondary Th1 act | 22.7 | HUVEC IL-1beta | 18.2 |
| Secondary Th2 act | 25.7 | HUVEC IFN gamma | 18.6 |
| Secondary Tr1 act | 37.9 | HUVEC TNF alpha + | 16.6 |
| | | IFN gamma | |
| Secondary Th1 rest | 15.7 | HUVEC TNF alpha + | 17.0 |
| | | IL4 | |
| Secondary Th2 rest | 11.2 | HUVEC IL-11 | 9.3 |
| Secondary Tr1 rest | 11.5 | Lung Microvascular EC | 14.2 |
| | | none | |
| Primary Th1 act | 16.6 | Lung Microvascular EC | 12.9 |
| | | TNF alpha + IL-1beta | |
| Primary Th2 act | 29.9 | Microvascular Dermal | 17.7 |
| | | EC none | |
| Primary Tr1 act | 44.1 | Microsvasular Dermal | 17.1 |
| | | EC TNF alpha + IL-1beta | |
| Primary Th1 rest | 69.7 | Bronchial epithelium | 6.8 |
| | | TNF alpha + IL-1beta | |
| Primary Th2 rest | 40.9 | Small airway epithelium | 8.5 |
| | | none | |
| Primary Tr1 rest | 24.5 | Small airway epithelium | 40.9 |
| | | TNF alpha + IL-1beta | |
| CD45RA CD4 | 15.6 | Coronery artery SMC rest | 9.4 |
| lymphocyte act | | | |
| CD45RO CD4 | 30.4 | Coronery artery SMC | 8.5 |
| lymphocyte act | | TNF alpha + IL-1beta | |
| CD8 lymphocyte act | 19.3 | Astrocytes rest | 16.3 |
| Secondary CD8 | 27.7 | Astrocytes TNF alpha + | 30.4 |
| lymphocyte rest | | IL-1beta | |
| Secondary CD8 | 16.3 | KU-812 (Basophil) rest | 26.8 |
| lymphocyte act | | | |
| CD4 lymphocyte none | 12.4 | KU-812 (Basophil) | 80.7 |
| | | PMA/ionomycin | |
| 2ry Th1/Th2/Tr1_anti- | 17.3 | CCD1106 | 13.6 |
| CD95 CH11 | | (Keratinocytes) none | |
| LAK cells rest | 10.7 | CCD1106 | 100.0 |
| | | (Keratinocytes) | |
| | | TNF alpha + IL-1beta | |
| LAK cells IL-2 | 30.4 | Liver cirrhosis | 10.4 |
| LAK cells IL-2 + IL-12 | 24.5 | Lupus kidney | 16.5 |
| LAK cells IL-2 + IFN | 34.2 | NCI-H292 none | 22.4 |
| gamma | | | |
| LAK cells IL-2 + IL-18 | 20.9 | NCI-H292 IL-4 | 36.9 |
| LAK cells | 13.3 | NCI-H292 IL-9 | 27.5 |
| PMA/ionomycin | | | |
| NK Cells IL-2 rest | 15.6 | NCI-H292 IL-13 | 15.9 |
| Two Way MLR 3 day | 19.5 | NCI-H292 IFN gamma | 15.9 |
| Two Way MLR 5 day | 16.2 | HPAEC none | 9.3 |
| Two Way MLR 7 day | 15.1 | HPAEC TNF alpha + IL- | 18.9 |
| | | 1beta | |
| PBMC rest | 13.1 | Lung fibroblast none | 20.4 |
| PBMC PWM | 26.6 | Lung fibroblast TNF | 20.9 |
| | | alpha + IL-1beta | |
| PBMC PHA-L | 9.6 | Lung fibroblast IL-4 | 19.5 |
| Ramos (B cell) none | 39.2 | Lung fibroblast IL-9 | 12.2 |
| Ramos (B cell) | 37.9 | Lung fibroblast IL-13 | 10.5 |
| ionomycin | | | |
| B lymphocytes PWM | 31.4 | Lung fibroblast IFN | 29.1 |
| | | gamma | |
| B lymphocytes CD40L | 32.3 | Dermal fibroblast | 26.8 |
| and IL-4 | | CCD1070 rest | |
| EOL-1 dbcAMP | 18.7 | Dermal fibroblast | 46.3 |
| | | CCD1070 TNF alpha | |
| EOL-1 dbcAMP | 45.7 | Dermal fibroblast | 16.0 |
| PMA/ionomycin | | CCD1070 IL-1beta | |
| Dendritic cells none | 12.8 | Dermal fibroblast IFN | 7.5 |
| | | gamma | |
| Dendritic cells LPS | 9.6 | Dermal fibroblast IL-4 | 17.1 |
| Dendritic cells anti- | 14.6 | IBD Colitis 2 | 4.2 |
| CD40 | | | |
| Monocytes rest | 20.6 | IBD Crohn's | 2.9 |
| Monocytes LPS | 20.9 | Colon | 49.0 |
| Macrophages rest | 15.8 | Lung | 11.1 |
| Macrophages LPS | 12.9 | Thymus | 20.2 |
| HUVEC none | 24.3 | Kidney | 33.2 |
| HUVEC starved | 33.7 | | |

CNS_neurodegeneration_v1.0 Summary: Ag3443 This panel confirms the expression of the CG59452-01 gene at significant levels in the brain in an independent group of individuals. However, no differential expression of this gene was detected between Alzheimer's diseased postmortem brains and those of non-demented controls in this experiment. Expression of this gene in the brain suggests that it may play a role in central nervous system disorders other than Alzheimer's disease, such as Parkinson's disease, epilepsy, multiple sclerosis, schizophrenia and depression.

General_screening_panel_v1.4 Summary: Ag3443 The amp plot indicates that there were experimental difficulties with this run. (Data not shown).

Panel 4D Summary: Ag3443 Highest expression of the CG59452-01 gene is detected in TNFalpha+IL-1 beta treated keratinocytes and PMA/ionomycin treated KU-812 basophil cells (CTs=24.5). Thus, antibody or small molecule therapies designed with the protein encoded for by this gene could block or inhibit inflammation or tissue damage due to basophil activation in response to asthma, allergies, hypersensitivity reactions, psoriasis, and viral infections.

BW. CG59572-01 and CG59572-02: Pseudouridine Synthase 3

Expression of gene CG59572-01 and CG59572-02 was assessed using the primer-probe set Ag3476, described in Table BWA. Results of the RTQ-PCR runs are shown in Tables BWB, BWC and BWD. Please note that CG59572-02 represents a full-length physical clone of the CG59572-01 gene, validating the prediction of the gene sequence.

TABLE BWA

Probe Name Ag3476

| | | | Start | SEQ ID |
|---|---|---|---|---|
| Primers | Sequences | Length | Position | NO: |
| Forward | 5′-acctacaacaactgtgggctaa-3′ | 22 | 1070 | 626 |
| Probe | TET-5′-tcatgctgtcaaaactcacatgttgt-3′-TAMRA | 26 | 1092 | 627 |
| Reverse | 5′-ggaacagtgtccagtccttgta-3′ | 22 | 1127 | 628 |

TABLE BWB
CNS_neurodegeneration_v1.0

| | Rel. Exp. | | |
|---|---|---|---|
| | (%) Ag3476, | | Rel. Exp. (%) |
| | Run | | Ag3476, Run |
| Tissue Name | 210377171 | Tissue Name | 210377171 |
| AD 1 Hippo | 16.7 | Control (Path) 3 | 4.5 |
| | | Temporal Ctx | |
| AD 2 Hippo | 23.8 | Control (Path) 4 | 31.6 |
| | | Temporal Ctx | |
| AD 3 Hippo | 10.3 | AD 1 Occipital | 28.5 |
| | | Ctx | |
| AD 4 Hippo | 8.5 | AD 2 Occipital | 0.0 |
| | | Ctx (Missing) | |
| AD 5 hippo | 88.3 | AD 3 Occipital | 7.9 |
| | | Ctx | |
| AD 6 Hippo | 77.4 | AD 4 Occipital | 20.6 |
| | | Ctx | |
| Control 2 Hippo | 37.4 | AD 5 Occipital | 35.4 |
| | | Ctx | |
| Control 4 Hippo | 9.6 | AD 6 Occipital | 54.7 |
| | | Ctx | |
| Control (Path) 3 | 8.4 | Control 1 Occipital | 4.7 |
| Hippo | | Ctx | |
| AD 1 Temporal Ctx | 18.6 | Control 2 Occipital | 67.8 |
| | | Ctx | |
| AD 2 Temporal Ctx | 32.3 | Control 3 Occipital | 15.6 |
| | | Ctx | |
| AD 3 Temporal Ctx | 8.0 | Control 4 Occipital | 5.6 |
| | | Ctx | |
| AD 4 Temporal Ctx | 21.6 | Control (Path) 1 | 100.0 |
| | | Occipital Ctx | |
| AD 5 Inf Temporal | 84.1 | Control (Path) 2 | 7.8 |
| Ctx | | Occipital Ctx | |
| AD 5 SupTemporal | 41.5 | Control (Path) 3 | 5.9 |
| Ctx | | Occipital Ctx | |
| AD 6 Inf Temporal | 77.4 | Control (Path) 4 | 18.0 |
| Ctx | | Occipital Ctx | |
| AD 6 Sup Temporal | 88.3 | Control 1 Parietal | 8.1 |
| Ctx | | Ctx | |
| Control 1 Temporal | 5.4 | Control 2 Parietal | 47.0 |
| Ctx | | Ctx | |
| Control 2 Temporal | 40.1 | Control 3 Parietal | 18.2 |
| Ctx | | Ctx | |
| Control 3 Temporal | 22.1 | Control (Path) 1 | 71.7 |
| Ctx | | Parietal Ctx | |
| Control 4 Temporal | 5.1 | Control (Path) 2 | 20.6 |
| Ctx | | Parietal Ctx | |
| Control (Path) 1 | 55.1 | Control (Path) 3 | 5.3 |
| Temporal Ctx | | Parietal Ctx | |
| Control (Path) 2 | 31.6 | Control (Path) 4 | 46.3 |
| Temporal Ctx | | Parietal Ctx | |

TABLE BWC
General_screening_panel_v1.4

| | Rel. Exp. | | Rel. Exp. |
|---|---|---|---|
| | (%) Ag3476, | | (%) Ag3476, |
| | Run | | Run |
| Tissue Name | 217119004 | Tissue Name | 217119004 |
| Adipose | 5.7 | Renal ca. TK-10 | 13.9 |
| Melanoma* | 13.4 | Bladder | 16.3 |
| Hs688(A).T | | | |
| Melanoma* | 14.6 | Gastric ca. (liver met.) | 42.9 |
| Hs688(B).T | | NCI-N87 | |
| Melanoma* M14 | 18.6 | Gastric ca. KATO III | 92.0 |
| Melanoma* | 38.7 | Colon ca. SW-948 | 7.3 |
| LOXIMVI | | | |
| Melanoma* SK- | 27.0 | Colon ca. SW480 | 40.9 |
| MEL-5 | | | |
| Squamous cell | 9.9 | Colon ca.* (SW480 | 27.5 |
| carcinoma SCC-4 | | met) SW620 | |
| Testis Pool | 5.9 | Colon ca. HT29 | 24.0 |
| Prostate ca.* (bone | 34.4 | Colon ca. HCT-116 | 32.5 |
| met) PC-3 | | | |
| Prostate Pool | 5.6 | Colon ca. CaCo-2 | 52.1 |
| Placenta | 1.1 | Colon cancer tissue | 10.4 |
| Uterus Pool | 3.8 | Colon ca. SW1116 | 3.9 |
| Ovarian ca. | 15.4 | Colon ca. Colo-205 | 6.1 |
| OVCAR-3 | | | |
| Ovarian ca. SK- | 20.7 | Colon ca. SW-48 | 9.0 |
| OV-3 | | | |
| Ovarian ca. | 14.5 | Colon Pool | 13.6 |
| OVCAR-4 | | | |
| Ovarian ca. | 44.8 | Small Intestine Pool | 8.4 |
| OVCAR-5 | | | |
| Ovarian ca. | 18.4 | Stomach Pool | 7.0 |
| IGROV-1 | | | |
| Ovarian ca. | 10.3 | Bone Marrow Pool | 4.0 |
| OVCAR-8 | | | |
| Ovary | 7.0 | Fetal Heart | 6.3 |
| Breast ca. MCF-7 | 16.8 | Heart Pool | 5.4 |
| Breast ca. MDA- | 31.0 | Lymph Node Pool | 14.5 |
| MB-231 | | | |
| Breast ca. BT 549 | 51.1 | Fetal Skeletal Muscle | 4.2 |
| Breast ca. T47D | 100.0 | Skeletal Muscle Pool | 10.7 |
| Breast ca. MDA-N | 28.7 | Spleen Pool | 5.4 |
| Breast Pool | 11.0 | Thymus Pool | 8.2 |
| Trachea | 6.2 | CNS cancer | 11.9 |
| | | (glio/astro) U87-MG | |
| Lung | 2.0 | CNS cancer | 44.1 |
| | | (glio/astro) U-118-MG | |
| Fetal Lung | 15.5 | CNS cancer | 29.9 |
| | | (neuro; met) SK-N-AS | |
| Lung ca. NCI-N417 | 14.5 | CNS cancer (astro) | 13.6 |
| | | SF-539 | |
| Lung ca. LX-1 | 31.0 | CNS cancer (astro) | 43.8 |
| | | SNB-75 | |
| Lung ca. NCI-H146 | 9.3 | CNS cancer (glio) | 16.0 |
| | | SNB-19 | |
| Lung ca. SHP-77 | 36.6 | CNS cancer (glio) SF- | 44.8 |
| | | 295 | |
| Lung ca. A549 | 14.4 | Brain (Amygdala) | 3.6 |
| | | Pool | |
| Lung ca. NCI-H526 | 21.6 | Brain (cerebellum) | 7.8 |
| Lung ca. NCI-H23 | 23.0 | Brain (fetal) | 7.6 |
| Lung ca. NCI-H460 | 13.9 | Brain (Hippocampus) | 4.5 |
| | | Pool | |
| Lung ca. HOP-62 | 14.9 | Cerebral Cortex Pool | 6.3 |
| Lung ca. NCI-H522 | 38.7 | Brain (Substantia | 3.4 |
| | | nigra) Pool | |
| Liver | 1.9 | Brain (Thalamus) Pool | 6.9 |
| Fetal Liver | 17.2 | Brain (whole) | 3.8 |
| Liver ca. HepG2 | 18.4 | Spinal Cord Pool | 3.6 |
| Kidney Pool | 12.9 | Adrenal Gland | 3.8 |
| Fetal Kidney | 15.3 | Pituitary gland Pool | 3.2 |
| Renal ca. 786-0 | 13.4 | Salivary Gland | 1.8 |
| Renal ca. A498 | 7.1 | Thyroid (female) | 4.4 |
| Renal ca. ACHN | 10.4 | Pancreatic ca. | 27.7 |
| | | CAPAN2 | |
| Renal ca. UO-31 | 22.1 | Pancreas Pool | 14.7 |

TABLE BWD
Panel 4D

| | Rel. Exp. (%) | | Rel. Exp. (%) |
|---|---|---|---|
| | Ag3476, Run | | Ag3476, Run |
| Tissue Name | 166420471 | Tissue Name | 166420471 |
| Secondary Th1 act | 31.4 | HUVEC IL-1beta | 27.5 |
| Secondary Th2 act | 30.6 | HUVEC IFN gamma | 23.7 |
| Secondary Tr1 act | 40.3 | HUVEC TNF alpha + | 15.4 |
| | | IFN gamma | |
| Secondary Th1 rest | 11.6 | HUVEC TNF alpha + | 16.2 |
| | | IL4 | |
| Secondary Th2 rest | 9.3 | HUVEC IL-11 | 13.4 |
| Secondary Tr1 rest | 9.2 | Lung Microvascular EC | 12.3 |
| | | none | |
| Primary Th1 act | 23.5 | Lung Microvascular EC | 11.0 |
| | | TNF alpha + IL-1beta | |
| Primary Th2 act | 44.8 | Microvascular Dermal | 20.3 |
| | | EC none | |
| Primary Tr1 act | 55.5 | Microsvasular Dermal | 13.6 |
| | | EC TNF alpha + IL-1beta | |
| Primary Th1 rest | 48.6 | Bronchial epithelium | 7.8 |
| | | TNF alpha + IL1beta | |
| Primary Th2 rest | 24.7 | Small airway epithelium | 9.5 |
| | | none | |
| Primary Tr1 rest | 22.8 | Small airway epithelium | 48.0 |
| | | TNF alpha + IL-1beta | |
| CD45RA CD4 | 15.9 | Coronery artery SMC rest | 14.1 |
| lymphocyte act | | | |
| CD45RO CD4 | 45.1 | Coronery artery SMC | 9.8 |
| lymphocyte act | | TNF alpha + IL-1beta | |
| CD8 lymphocyte act | 26.2 | Astrocytes rest | 13.9 |
| Secondary CD8 | 46.3 | Astrocytes TNF alpha + | 14.2 |
| lymphocyte rest | | IL-1beta | |
| Secondary CD8 | 23.0 | KU-812 (Basophil) rest | 29.5 |
| lymphocyte act | | | |
| CD4 lymphocyte none | 6.3 | KU-812 (Basophil) | 55.1 |
| | | PMA/ionomycin | |
| 2ry Th1/Th2/Tr1_anti- | 15.8 | CCD1106 | 18.7 |
| CD95 CH11 | | (Keratinocytes) none | |
| LAK cells rest | 8.4 | CCD1106 | 100.0 |
| | | (Keratinocytes) | |
| | | TNF alpha + IL-1beta | |
| LAK cells IL-2 | 29.1 | Liver cirrhosis | 12.8 |
| LAK cells IL-2 + IL-12 | 35.8 | Lupus kidney | 7.9 |
| LAK cells IL-2 + IFN | 42.6 | NCI-H292 none | 44.4 |
| gamma | | | |
| LAK cells IL-2 + IL-18 | 28.7 | NCI-H292 IL-4 | 52.5 |
| LAK cells | 14.0 | NCI-H292 IL-9 | 55.5 |
| PMA/ionomycin | | | |
| NK Cells IL-2 rest | 19.9 | NCI-H292 IL-13 | 29.3 |
| Two Way MLR 3 day | 23.0 | NCI-H292 IFN gamma | 30.1 |
| Two Way MLR 5 day | 31.0 | HPAEC none | 10.7 |
| Two Way MLR 7 day | 18.0 | HPAEC TNF alpha + IL- | 13.1 |
| | | 1beta | |
| PBMC rest | 4.8 | Lung fibroblast none | 23.3 |
| PBMC PWM | 39.5 | Lung fibroblast TNF | 16.0 |
| | | alpha + IL-1beta | |
| PBMC PHA-L | 11.4 | Lung fibroblast IL-4 | 27.7 |
| Ramos (B cell) none | 18.2 | Lung fibroblast IL-9 | 22.5 |
| Ramos (B cell) | 16.2 | Lung fibroblast IL-13 | 19.6 |
| ionomycin | | | |
| B lymphocytes PWM | 56.3 | Lung fibroblast IFN | 48.3 |
| | | gamma | |
| B lymphocytes CD40L | 36.9 | Dermal fibroblast | 28.5 |
| and IL-4 | | CCD1070 rest | |
| EOL-1 dbcAMP | 23.5 | Dermal fibroblast | 38.7 |
| | | CCD1070 TNF alpha | |
| EOL-1 dbcAMP | 20.0 | Dermal fibroblast | 11.8 |
| PMA/ionomycin | | CCD1070 IL-1beta | |
| Dendritic cells none | 10.1 | Dermal fibroblast IFN | 9.9 |
| | | gamma | |
| Dendritic cells LPS | 9.7 | Dermal fibroblast IL-4 | 15.7 |
| Dendritic cells anti- | 10.2 | IBD Colitis 2 | 6.6 |
| CD40 | | | |
| Monocytes rest | 12.0 | IBD Crohn's | 6.2 |
| Monocytes LPS | 12.5 | Colon | 46.0 |
| Macrophages rest | 13.3 | Lung | 12.6 |
| Macrophages LPS | 7.4 | Thymus | 21.8 |
| HUVEC none | 24.3 | Kidney | 18.3 |
| HUVEC starved | 33.4 | | |

CNS_neurodegeneration_v1.0 Summary: Ag3476 This panel confirms the expression of the CG59572-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3476 Highest expression of the CG59572-01 gene is detected in a breast cancer cell line sample (CT=27.4). Furthermore, moderate to high expression of this gene is detected in CNS cancer, colon cancer, gastric cancer, pancreatic cancer, lung cancer, ovarian cancer, and prostate cancer. Therefore, therapeutic modulation of the activity of this gene or its protein product, through the use of small molecule drugs, protein therapeutics or antibodies, might be beneficial in the treatment of these cancers.

This gene is expressed at low to moderate levels throughout the CNS, including in amygdala, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

In addition, this gene is expressed at much higher levels in fetal lung and liver tissue (CTs=30) when compared to expression in the adult counterpart (CTs=33). Thus, expression of this gene may be used to differentiate between the fetal and adult source of these tissues.

Panel 4D Summary: Ag3476 Highest expression of the CG59572-01 gene is detected in TNFalpha+IL-1 beta treated keratinocytes (CT=27.2). Expression of this gene appears to be stimulated in activated secondary Th1, Th2 and Tr1 cells, PWM treated PBMCs, PWM treated 13-lymphocytes, IL-2/IL-2+IL-12/IL-2+IFN gamma/IL-2+IL-18 treated LAK cells, and TNFalpha+IL-1beta treated small airway epithelium (CTs=28-30). Thus, this gene may be important in the activation of T and B cells or the function of activated T and B cells. Therefore, small molecules that antagonize the function of this gene product may reduce or eliminate the symptoms in patients with autoimmune and inflammatory diseases in which B and T cells play a part in the initiation or progression of the disease process, such as systemic lupus erythematosus, Crohn's disease, ulcerative colitis, multiple sclerosis, chronic obstructive pulmonary disease, asthma, emphysema, rheumatoid arthritis, or psoriasis.

BX. CG59522-01: Myosin I

Expression of gene CG59522-01 was assessed using the primer-probe set Ag3456, described in Table BXA. Results of the RTQ-PCR runs are shown in Table BXB.

TABLE BXA

Probe Name 3456

| | | | Start | SEQ ID |
|---|---|---|---|---|
| Primers | Sequences | Length | Position | NO: |
| Forward | 5′-atgaactgcacttggagagaaa-3′ | 22 | 664 | 629 |
| Probe | TET-5′-aatttcacacaccagggagcaggact-3′-TAMRA | 26 | 699 | 630 |
| Reverse | 5′-ctctgctcatcactcacagtca-3′ | 22 | 730 | 631 |

TABLE BXB
Panel 4D

| | Rel. Exp. (%) | | Rel. Exp. (%) |
|---|---|---|---|
| | Ag3456, Run | | Ag3456, Run |
| Tissue Name | 166397214 | Tissue Name | 166397214 |
| Secondary Th1 act | 20.2 | HUVEC IL-1beta | 0.0 |
| Secondary Th2 act | 19.6 | HUVEC IFN gamma | 0.0 |
| Secondary Tr1 act | 35.4 | HUVEC TNF alpha + | 0.0 |
| | | IFN gamma | |
| Secondary Th1 rest | 34.2 | HUVEC TNF alpha + | 0.0 |
| | | IL4 | |
| Secondary Th2 rest | 17.3 | HUVEC IL-11 | 0.0 |
| Secondary Tr1 rest | 20.7 | Lung Microvascular EC | 0.0 |
| | | none | |
| Primary Th1 act | 10.6 | Lung Microvascular EC | 0.0 |
| | | TNF alpha + IL-1beta | |
| Primary Th2 act | 13.3 | Microvascular Dermal | 0.0 |
| | | EC none | |
| Primary Tr1 act | 25.2 | Microsvasular Dermal | 0.0 |
| | | EC TNF alpha + IL-1beta | |
| Primary Th1 rest | 100.0 | Bronchial epithelium | 0.0 |
| | | TNF alpha + IL1beta | |
| Primary Th2 rest | 35.1 | Small airway epithelium | 0.0 |
| | | none | |
| Primary Tr1 rest | 25.9 | Small airway epithelium | 0.0 |
| | | TNF alpha + IL-1beta | |
| CD45RA CD4 | 9.9 | Coronery artery SMC rest | 0.0 |
| lymphocyte act | | | |
| CD45RO CD4 | 28.9 | Coronery artery SMC | 0.0 |
| lymphocyte act | | TNF alpha + IL-1beta | |
| CD8 lymphocyte act | 27.5 | Astrocytes rest | 0.0 |
| Secondary CD8 | 15.6 | Astrocytes TNF alpha + | 0.0 |
| lymphocyte rest | | IL-1beta | |
| Secondary CD8 | 26.6 | KU-812 (Basophil) rest | 2.2 |
| lymphocyte act | | | |
| CD4 lymphocyte none | 5.0 | KU-812 (Basophil) | 5.2 |
| | | PMA/ionomycin | |
| 2ry Th1/Th2/Tr1_anti- | 31.2 | CCD1106 | 0.0 |
| CD95 CH11 | | (Keratinocytes) none | |
| LAK cells rest | 4.7 | CCD1106 | 1.0 |
| | | (Keratinocytes) | |
| | | TNF alpha + IL-1beta | |
| LAK cells IL-2 | 41.5 | Liver cirrhosis | 0.8 |
| LAK cells IL-2 + IL-12 | 22.4 | Lupus kidney | 0.4 |
| LAK cells IL-2 + IFN | 29.5 | NCI-H292 none | 0.3 |
| gamma | | | |
| LAK cells IL-2 + IL-18 | 25.7 | NCI-H292 IL-4 | 0.1 |
| LAK cells | 4.4 | NCI-H292 IL-9 | 0.0 |
| PMA/ionomycin | | | |
| NK Cells IL-2 rest | 22.2 | NCI-H292 IL-13 | 0.0 |
| Two Way MLR 3 day | 14.2 | NCI-H292 IFN gamma | 0.0 |
| Two Way MLR 5 day | 16.0 | HPAEC none | 0.0 |
| Two Way MLR 7 day | 17.2 | HPAEC TNF alpha + IL- | 0.0 |
| | | 1beta | |
| PBMC rest | 7.5 | Lung fibroblast none | 0.0 |
| PBMC PWM | 33.9 | Lung fibroblast TNF | 0.0 |
| | | alpha + IL-1beta | |
| PBMC PHA-L | 12.7 | Lung fibroblast IL-4 | 0.0 |
| Ramos (B cell) none | 4.7 | Lung fibroblast IL-9 | 0.0 |
| Ramos (B cell) | 8.5 | Lung fibroblast IL-13 | 0.1 |
| ionomycin | | | |
| B lymphocytes PWM | 25.5 | Lung fibroblast IFN | 0.0 |
| | | gamma | |
| B lymphocytes CD40L | 18.8 | Dermal fibroblast | 0.0 |
| and IL-4 | | CCD1070 rest | |
| EOL-1 dbcAMP | 17.3 | Dermal fibroblast | 43.5 |
| | | CCD1070 TNF alpha | |
| EOL-1 dbcAMP | 9.9 | Dermal fibroblast | 0.0 |
| PMA/ionomycin | | CCD1070 IL-1beta | |
| Dendritic cells none | 2.1 | Dermal fibroblast IFN | 0.0 |
| | | gamma | |
| Dendritic cells LPS | 16.4 | Dermal fibroblast IL-4 | 0.0 |
| Dendritic cells anti- | 1.6 | IBD Colitis 2 | 0.3 |
| CD40 | | | |
| Monocytes rest | 20.9 | IBD Crohn's | 0.1 |
| Monocytes LPS | 45.7 | Colon | 4.2 |
| Macrophages rest | 1.3 | Lung | 2.5 |
| Macrophages LPS | 16.3 | Thymus | 0.0 |
| HUVEC none | 0.0 | Kidney | 11.3 |
| HUVEC starved | 0.0 | | |

CNS_neurodegeneration_v1.0 Summary: Ag3456 Expression of CG59522-01 gene is low/undetectable (CTs>35) across all of the samples on this panel (data not shown).

General_screening_panel_v1.4 Summary: Ag3456 Expression of CG59522-01 gene is low/undetectable (CTs>35) across all of the samples on this panel (data not shown).

Panel 4D Summary: Ag3456 Highest expression of the CG59522-01 gene is detected in sample derived from resting primary Th1 cells (CT=29.8). Thus, expression of this gene can be used to distinguish this sample from other samples in this panel. This gene is also expressed at low but significant levels in T cells prepared under a number of conditions, LAK cells, macrophages and dendritic cells also express the transcript. The only non-hematopoietic cell type that expresses the transcript detected by this primer and probe at significant levels is dermal fibroblasts. Colon and kidney also express low levels of the transcript. Thus, this transcript or the protein it encodes could be used to detect hematopoietically-derived cells. Furthermore, therapeutics designed with the protein encoded by this transcript could be important in the regulation the function of antigen presenting cells (macrophages and dendritic cells) or T cells and be important in the treatment of asthma, emphysema, psoriasis, arthritis, and IBD. Therefore, therapeutics designed with the protein encoded for by this transcript could be important in regulating T cell function and treating T and B cell mediated diseases such as asthma, arthritis, psoriasis, IBD, and systemic lupus erythematosus.

BY. CG59520-01: Farnesyl Pyrophosphate Synthetase

Expression of gene CG59520-01 was assessed using the primer-probe set Ag5923, described in Table BYA. Results of the RTQ-PCR runs are shown in Tables BYB and BYC.

TABLE BYA

Probe Name Ag5923

| | | | | |
|---|---|---|---|---|
| | | | Start | SEQ ID |
| Primers | Sequences | Length | Position | NO: |

| | | | | |
|---|---|---|---|---|
| Forward | 5′-gaatgggaaaccagaaatcag-3′ | 21 | 5 | 632 |
| Probe | TET-5′-tttatgcccaagcaaagcaggatttc-3′-TAMRA | 26 | 29 | 633 |
| Reverse | 5′-accctaacgatctgggagtagt-3′ | 22 | 62 | 634 |

TABLE BYB
General_screening panel v1.5

| | Rel. Exp. | | Rel. Exp. |
|---|---|---|---|
| | (%) Ag5923, | | (%) Ag5923, |
| | Run | | Run |
| Tissue Name | 247608956 | Tissue Name | 247608956 |
| Adipose | 0.6 | Renal ca. TK-10 | 12.1 |
| Melanoma* | 1.9 | Bladder | 7.1 |
| Hs688(A).T | | | |
| Melanoma* | 2.9 | Gastric ca. (liver met.) | 26.2 |
| Hs688(B).T | | NCI-N87 | |
| Melanoma* M14 | 8.9 | Gastric ca. KATO III | 31.0 |
| Melanoma* | 3.9 | Colon ca. SW-948 | 6.8 |
| LOXIMVI | | | |
| Melanoma* SK- | 2.4 | Colon ca. SW480 | 15.3 |
| MEL-5 | | | |
| Squamous cell | 1.8 | Colon ca.* (SW480 | 8.2 |
| carcinoma SCC-4 | | met) SW620 | |
| Testis Pool | 15.1 | Colon ca. HT29 | 1.9 |
| Prostate ca.* (bone | 5.6 | Colon ca. HCT-116 | 9.3 |
| met) PC-3 | | | |
| Prostate Pool | 2.3 | Colon ca. CaCo-2 | 6.4 |
| Placenta | 2.3 | Colon cancer tissue | 7.3 |
| Uterus Pool | 0.0 | Colon ca. SW1116 | 7.1 |
| Ovarian ca. | 6.7 | Colon ca. Colo-205 | 9.5 |
| OVCAR-3 | | | |
| Ovarian ca. SK- | 14.8 | Colon ca. SW-48 | 3.8 |
| OV-3 | | | |
| Ovarian ca. | 4.2 | Colon Pool | 5.6 |
| OVCAR-4 | | | |
| Ovarian ca. | 23.0 | Small Intestine Pool | 7.0 |
| OVCAR-5 | | | |
| Ovarian ca. | 4.1 | Stomach Pool | 0.6 |
| IGROV-1 | | | |
| Ovarian ca. | 0.7 | Bone Marrow Pool | 2.1 |
| OVCAR-8 | | | |
| Ovary | 12.1 | Fetal Heart | 4.1 |
| Breast ca. MCF-7 | 4.4 | Heart Pool | 1.1 |
| Breast ca. MDA- | 6.8 | Lymph Node Pool | 7.3 |
| MB-231 | | | |
| Breast ca. BT 549 | 17.8 | Fetal Skeletal Muscle | 1.0 |
| Breast ca. T47D | 5.6 | Skeletal Muscle Pool | 5.3 |
| Breast ca. MDA-N | 2.5 | Spleen Pool | 2.0 |
| Breast Pool | 9.5 | Thymus Pool | 8.0 |
| Trachea | 14.1 | CNS cancer | 1.7 |
| | | (glio/astro) U87-MG | |
| Lung | 13.9 | CNS cancer | 10.2 |
| | | (glio/astro) U-118-MG | |
| Fetal Lung | 16.7 | CNS cancer | 2.8 |
| | | (neuro; met) SK-N-AS | |
| Lung ca. NCI-N417 | 1.7 | CNS cancer (astro) | 2.8 |
| | | SF-539 | |
| Lung ca. LX-1 | 12.8 | CNS cancer (astro) | 14.2 |
| | | SNB-75 | |
| Lung ca. NCI-H146 | 1.6 | CNS cancer (glio) | 3.9 |
| | | SNB-19 | |
| Lung ca. SHP-77 | 1.5 | CNS cancer (glio) SF- | 10.8 |
| | | 295 | |
| Lung ca. A549 | 11.0 | Brain (Amygdala) | 1.8 |
| | | Pool | |
| Lung ca. NCI-H526 | 0.5 | Brain (cerebellum) | 4.0 |
| Lung ca. NCI-H23 | 10.9 | Brain (fetal) | 4.6 |
| Lung ca. NCI-H460 | 4.1 | Brain (Hippocampus) | 3.3 |
| | | Pool | |
| Lung ca. HOP-62 | 2.3 | Cerebral Cortex Pool | 0.0 |
| Lung ca. NCI-H522 | 5.2 | Brain (Substantia | 3.2 |
| | | nigra) Pool | |
| Liver | 0.5 | Brain (Thalamus) Pool | 1.6 |
| Fetal Liver | 4.3 | Brain (whole) | 1.8 |
| Liver ca. HepG2 | 0.3 | Spinal Cord Pool | 1.7 |
| Kidney Pool | 13.6 | Adrenal Gland | 1.7 |
| Fetal Kidney | 7.1 | Pituitary gland Pool | 0.0 |
| Renal ca. 786-0 | 11.7 | Salivary Gland | 1.2 |
| Renal ca. A498 | 5.6 | Thyroid (female) | 0.5 |
| Renal ca. ACHN | 6.2 | Pancreatic ca. | 100.0 |
| | | CAPAN2 | |
| Renal ca. UO-31 | 14.0 | Pancreas Pool | 15.3 |

TABLE BYC
Panel 4.1D

| | Rel. Exp. (%) | | Rel. Exp. (%) |
|---|---|---|---|
| | Ag5923, Run | | Ag5923, Run |
| Tissue Name | 247579946 | Tissue Name | 247579946 |
| Secondary Th1 act | 14.6 | HUVEC IL-1beta | 5.3 |
| Secondary Th2 act | 36.6 | HUVEC IFN gamma | 12.1 |
| Secondary Tr1 act | 5.6 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 10.5 | HUVEC IL-11 | 6.7 |
| Secondary Tr1 rest | 0.0 | Lung Microvascular EC | 15.5 |
| | | none | |
| Primary Th1 act | 0.0 | Lung Microvascular EC | 2.4 |
| | | TNF alpha + IL-1beta | |
| Primary Th2 act | 11.1 | Microvascular Dermal | 0.0 |
| | | EC none | |
| Primary Tr1 act | 11.0 | Microsvasular Dermal | 5.0 |
| | | EC TNF alpha + IL-1beta | |
| Primary Th1 rest | 0.0 | Bronchial epithelium | 13.5 |
| | | TNF alpha + IL1beta | |
| Primary Th2 rest | 1.5 | Small airway epithelium | 7.2 |
| | | none | |
| Primary Tr1 rest | 1.3 | Small airway epithelium | 44.8 |
| | | TNF alpha + IL-1beta | |
| CD45RA CD4 | 13.5 | Coronery artery SMC rest | 1.6 |
| lymphocyte act | | | |
| CD45RO CD4 | 12.9 | Coronery artery SMC | 6.7 |
| lymphocyte act | | TNF alpha + IL-1beta | |
| CD8 lymphocyte act | 1.8 | Astrocytes rest | 1.5 |
| Secondary CD8 | 3.6 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 15.3 |
| | | PMA/ionomycin | |
| 2ry Th1/Th2/Tr1_anti- | 0.0 | CCD1106 | 21.5 |
| CD95 CH11 | | (Keratinocytes) none | |
| LAK cells rest | 15.9 | CCD1106 | 31.9 |
| | | (Keratinocytes) | |
| | | TNF alpha + IL-1beta | |
| LAK cells IL-2 | 3.5 | Liver cirrhosis | 4.0 |
| LAK cells IL-2 + IL-12 | 0.0 | NCI-H292 none | 61.1 |
| LAK cells IL-2 + IFN | 2.3 | NCI-H292 IL-4 | 100.0 |
| gamma | | | |
| LAK cells IL-2 + IL-18 | 0.0 | NCI-H292 IL-9 | 45.1 |
| LAK cells | 13.0 | NCI-H292 IL-13 | 47.0 |
| PMA/ionomycin | | | |
| NK Cells IL-2 rest | 37.1 | NCI-H292 IFN gamma | 14.5 |
| Two Way MLR 3 day | 4.9 | HAPEC none | 0.0 |
| Two Way MLR 5 day | 0.0 | HPAEC TNF alpha + IL- | 24.1 |
| | | 1beta | |
| Two Way MLR 7 day | 1.2 | Lung fibroblast none | 6.0 |
| PBMC rest | 0.0 | Lung fibroblast TNF | 3.7 |
| | | alpha + IL-1beta | |
| PBMC PWM | 0.0 | Lung fibroblast IL-4 | 1.0 |
| PBMC PHA-L | 0.0 | Lung fibroblast IL-9 | 8.7 |
| Ramos (B cell) none | 0.0 | Lung fibroblast IL-13 | 0.0 |
| Ramos (B cell) | 17.7 | Lung fibroblast IFN | 3.8 |
| ionomycin | | gamma | |
| B lymphocytes PWM | 11.4 | Dermal fibroblast | 5.8 |
| | | CCD1070 rest | |
| B lymphocytes CD40L | 23.2 | Dermal fibroblast | 21.3 |
| and IL-4 | | CCD1070 TNF alpha | |
| EOL-1 dbcAMP | 21.0 | Dermal fibroblast | 8.6 |
| | | CCD1070 IL-1beta | |
| EOL-1 dbcAMP | 3.5 | Dermal fibroblast IFN | 12.9 |
| PMA/ionomycin | | gamma | |
| Dendritic cells none | 11.7 | Dermal fibroblast IL-4 | 13.8 |
| Dendritic cells LPS | 8.4 | Dermal Fibroblasts rest | 0.0 |
| Dendritic cells anti- | 3.8 | Neutrophils TNFa + LPS | 1.5 |
| CD40 | | | |
| Monocytes rest | 0.0 | Neutrophils rest | 18.0 |
| Monocytes LPS | 57.8 | Colon | 0.0 |
| Macrophages rest | 1.7 | Lung | 0.0 |
| Macrophages LPS | 9.8 | Thymus | 8.8 |
| HUVEC none | 3.1 | Kidney | 4.0 |
| HUVEC starved | 2.5 | | |

CNS_neurodegeneration_v1.0 Summary: Ag5923 Expression of the CG59520-01 gene is low/undetectable (CTs>34.5) across all of the samples on this panel (data not shown).

General_screening_panel_v1.5 Summary: Ag5923 Highest expression of the CG59520-01 gene is detected in sample derived from a pancreatic cancer cell line (CT=31.5). Thus, expression of this gene can be used in distinguishing this sample from other samples from the panel and as a marker for pancreatic cancer. In addition low levels of expression of this gene are associated with samples derived from CNS, colon, gastric, renal, lung, breast, ovarian and melanoma cnacer cell lines. This gene encodes a farnesyl pyrophosphate synthetase, which is involved in cholesterol biosynthesis. It has been suggested that in several types of cancer, activation of p21 would be aided by continuous farnesylation due to stimulation of the cholesterol biosynthetic pathway in tumors (Rao, 1995). Therefore, therapeutic modulation of the activity of protein encoded by this gene may be beneficial in the treatment of these cancers.

In addition, low but significant levels of expression in the pancreas suggest that this gene product may be useful in the treatment of type II diabetes.

### REFERENCES

1. Rao K N. (1995) The significance of the cholesterol biosynthetic pathway in cell growth and carcinogenesis (review). Anticancer Res March-April 1995;15(2):309-14

Panel 4.1D Summary: Ag5923 High expression of the CG59520-01 gene is detected in sample derived from untreated and IL4 treated NCI-H292 cells (CTs=33). Thus, expression of this gene could be used to distinguish these samples from other samples from the panel. Also, therapeutic modulation of the activity of this gene product may be beneficial in the treatment asthma and emphysema.

Panel 5 Islet Summary: Ag5923 Expression of the CG59520-01 gene is low/undetectable (CTs>34.5) across all of the samples on this panel (data not shown).

BZ. CG-59704-01: Serine/Threonine Kinase

Expression of gene CG59704-01 was assessed using the primer-probe set Ag3509, described in Table BZA. Results of the RTQ-PCR runs are shown in Tables BZB, BZC and BZD.

TALE BZA

Probe Name Ag3509

| | | | Start | |
|---|---|---|---|---|
| Primers | Sequences | Length | Position | SEQ ID NO: |
| Forward | 5′-gacttccctcacctagcttctg-3′ | 22 | 4228 | 635 |
| Probe | TET-5′-actgcatgccaccactgctgagta-3′-TAMRA | 24 | 4257 | 636 |
| Reverse | 5′-caccaacctagcaaacaaacag-3′ | 22 | 4281 | 637 |

TABLE BZB
CNS_neurodegeneration_v1.0

| | | | Rel. Exp. |
|---|---|---|---|
| | Rel. Exp. (%) | | (%) Ag3509, |
| | Ag3509, Run | | Run |
| Tissue Name | 210499481 | Tissue Name | 210499481 |
| AD 1 Hippo | 20.2 | Control (Path) 3 | 24.1 |
| | | Temporal Ctx | |
| AD 2 Hippo | 20.2 | Control (Path) 4 | 20.7 |
| | | Temporal Ctx | |
| AD 3 Hippo | 23.3 | AD 1 Occipital Ctx | 16.4 |
| AD 4 Hippo | 13.5 | AD 2 Occipital Ctx | 0.0 |
| | | (Missing) | |
| AD 5 Hippo | 66.4 | AD 3 Occipital Ctx | 9.9 |
| AD 6 Hippo | 84.7 | AD 4 Occipital Ctx | 4.6 |
| Control 2 Hippo | 18.0 | AD 5 Occipital Ctx | 30.4 |
| Control 4 Hippo | 0.4 | AD 6 Occipital Ctx | 0.0 |
| Control (Path) 3 | 0.3 | Control 1 Occipital | 10.0 |
| Hippo | | Ctx | |
| AD 1 Temporal | 23.7 | Control 2 Occipital | 35.6 |
| Ctx | | Ctx | |
| AD 2 Temporal | 15.4 | Control 3 Occipital | 16.3 |
| Ctx | | Ctx | |
| AD 3 Temporal | 10.8 | Control 4 Occipital | 25.2 |
| Ctx | | Ctx | |
| AD 4 Temporal | 18.8 | Control (Path) 1 | 57.4 |
| Ctx | | Occipital Ctx | |
| AD 5 Inf Temporal | 100.0 | Control (Path) 2 | 8.1 |
| Ctx | | Occipital Ctx | |
| AD 5 Sup | 72.2 | Control (Path) 3 | 5.4 |
| Temporal Ctx | | Occipital Ctx | |
| AD 6 Inf Temporal | 21.9 | Control (Path) 4 | 30.4 |
| Ctx | | Occipital Ctx | |
| AD 6 Sup | 62.4 | Control 1 Parietal | 12.1 |
| Temporal Ctx | | Ctx | |
| Control 1 | 5.4 | Control 2 Parietal | 54.0 |
| Temporal Ctx | | Ctx | |
| Control 2 | 30.6 | Control 3 Parietal | 12.0 |
| Temporal Ctx | | Ctx | |
| Control 3 | 6.0 | Control (Path) 1 | 51.4 |
| Temporal Ctx | | Parietal Ctx | |
| Control 3 | 12.2 | Control (Path) 2 | 16.0 |
| Temporal Ctx | | Parietal Ctx | |
| Control (Path) 1 | 40.9 | Control (Path) 3 | 2.1 |
| Temporal Ctx | | Parietal Ctx | |
| Control (Path) 2 | 31.9 | Control (Path) 4 | 29.7 |
| Temporal Ctx | | Parietal Ctx | |

TABLE BZC
General_screening_panel_v1.4

| | Rel. Exp. | | Rel. Exp. |
|---|---|---|---|
| | (%) Ag3509, | | (%) Ag3509, |
| | Run | | Run |
| Tissue Name | 217240617 | Tissue Name | 217240617 |
| Adipose | 9.5 | Renal ca. TK-10 | 20.4 |
| Melanoma* | 8.7 | Bladder | 16.6 |
| Hs688(A).T | | | |
| Melanoma* | 4.5 | Gastric ca. (liver met.) | 37.6 |
| Hs688(B).T | | NCI-N87 | |
| Melanoma* M14 | 12.0 | Gastric ca. KATO III | 23.3 |
| Melanoma* | 3.1 | Colon ca. SW-948 | 6.9 |
| LOXIMVI | | | |
| Melanoma* SK- | 11.0 | Colon ca. SW480 | 35.8 |
| MEL-5 | | | |
| Squamous cell | 9.5 | Colon ca.* (SW480 | 24.7 |
| carcinoma SCC-4 | | met) SW620 | |
| Testis Pool | 5.4 | Colon ca. HT29 | 9.3 |
| Prostate ca.* (bone | 24.1 | Colon ca. HCT-116 | 62.4 |
| met) PC-3 | | | |
| Prostate Pool | 4.5 | Colon ca. CaCo-2 | 8.8 |
| Placenta | 1.9 | Colon cancer tissue | 10.9 |
| Uterus Pool | 1.8 | Colon ca. SW1116 | 3.7 |
| Ovarian ca. | 43.2 | Colon ca. Colo-205 | 1.2 |
| OVCAR-3 | | | |
| Ovarian ca. SK- | 42.3 | Colon ca. SW-48 | 2.8 |
| OV-3 | | | |
| Ovarian ca. | 1.1 | Colon Pool | 9.2 |
| OVCAR-4 | | | |
| Ovarian ca. | 19.1 | Small Intestine Pool | 8.2 |
| OVCAR-5 | | | |
| Ovarian ca. | 8.7 | Stomach Pool | 3.7 |
| IGROV-1 | | | |
| Ovarian ca. | 7.5 | Bone Marrow Pool | 4.6 |
| OVCAR-8 | | | |
| Ovary | 3.4 | Fetal Heart | 7.3 |
| Breast ca. MCF-7 | 20.7 | Heart Pool | 5.6 |
| Breast ca. MDA- | 32.8 | Lymph Node Pool | 10.1 |
| MB-231 | | | |
| Breast ca. BT 549 | 35.1 | Fetal Skeletal Muscle | 6.7 |
| Breast ca. T47D | 34.4 | Skeletal Muscle Pool | 3.6 |
| Breast ca. MDA-N | 18.6 | Spleen Pool | 7.7 |
| Breast Pool | 15.7 | Thymus Pool | 11.0 |
| Trachea | 6.8 | CNS cancer | 27.4 |
| | | (glio/astro) U87-MG | |
| Lung | 15.3 | CNS cancer | 45.4 |
| | | (glio/astro) U-118-MG | |
| Fetal Lung | 17.6 | CNS cancer | 16.3 |
| | | (neuro; met) SK-N-AS | |
| Lung ca. NCI-N417 | 11.8 | CNS cancer (astro) | 9.9 |
| | | SF-539 | |
| Lung ca. LX-1 | 29.5 | CNS cancer (astro) | 50.7 |
| | | SNB-75 | |
| Lung ca. NCI-H146 | 5.6 | CNS cancer (glio) | 7.0 |
| | | SNB-19 | |
| Lung ca. SHP-77 | 19.1 | CNS cancer (glio) SF- | 25.7 |
| | | 295 | |
| Lung ca. A549 | 17.2 | Brain (Amygdala) | 4.9 |
| | | Pool | |
| Lung ca. NCI-H526 | 4.2 | Brain (cerebellum) | 13.7 |
| Lung ca. NCI-H23 | 100.0 | Brain (fetal) | 14.8 |
| Lung ca. NCI-H460 | 27.2 | Brain (Hippocampus) | 3.7 |
| | | Pool | |
| Lung ca. HOP-62 | 16.2 | Cerebral Cortex Pool | 3.0 |
| Lung ca. NCI-H522 | 29.9 | Brain (Substantia | 1.7 |
| | | nigra) Pool | |
| Liver | 0.0 | Brain (Thalamus) Pool | 5.6 |
| Fetal Liver | 10.1 | Brain (whole) | 6.1 |
| Liver ca. HepG2 | 17.7 | Spinal Cord Pool | 2.0 |
| Kidney Pool | 26.1 | Adrenal Gland | 8.8 |
| Fetal Kidney | 24.3 | Pituitary gland Pool | 3.4 |
| Renal ca. 786-0 | 19.8 | Salivary Gland | 4.9 |
| Renal ca. A498 | 2.4 | Thyroid (female) | 3.4 |
| Renal ca. ACHN | 8.2 | Pancreatic ca. | 34.2 |
| | | CAPAN2 | |
| Renal ca. UO-31 | 9.5 | Pancreas Pool | 16.7 |

TABLE BZD
Panel 4D

| | Rel. Exp. (%) | | Rel. Exp. (%) |
|---|---|---|---|
| | Ag3509, Run | | Ag3509, Run |
| Tissue Name | 166407201 | Tissue Name | 166407201 |
| Secondary Th1 act | 50.0 | HUVEC IL-1beta | 4.7 |
| Secondary Th2 act | 45.4 | HUVEC IFN gamma | 4.8 |
| Secondary Tr1 act | 87.7 | HUVEC TNF alpha + | 10.0 |
| | | IFN gamma | |
| Secondary Th1 rest | 17.4 | HUVEC TNF alpha + | 19.9 |
| | | IL4 | |
| Secondary Th2 rest | 21.9 | HUVEC IL-11 | 13.4 |
| Secondary Tr1 rest | 32.5 | Lung Microvascular EC | 14.1 |
| | | none | |
| Primary Th1 act | 40.9 | Lung Microvascular EC | 24.8 |
| | | TNF alpha + IL-1beta | |
| Primary Th2 act | 83.5 | Microvascular Dermal | 22.1 |
| | | EC none | |
| Primary Tr1 act | 100.0 | Microsvasular Dermal | 12.2 |
| | | EC TNF alpha + IL-1beta | |
| Primary Th1 rest | 75.8 | Bronchial epithelium | 9.9 |
| | | TNF alpha + IL1beta | |
| Primary Th2 rest | 31.0 | Small airway epithelium | 7.7 |
| | | none | |
| Primary Tr1 rest | 41.5 | Small airway epithelium | 23.0 |
| | | TNF alpha + IL-1beta | |
| CD45RA CD4 | 21.9 | Coronery artery SMC rest | 7.6 |
| lymphocyte act | | | |
| CD45RO CD4 | 56.6 | Coronery artery SMC | 2.0 |
| lymphocyte act | | TNF alpha + IL-1beta | |
| CD8 lymphocyte act | 58.6 | Astrocytes rest | 25.5 |
| Secondary CD8 | 51.4 | Astrocytes TNF alpha + | 28.5 |
| lymphocyte rest | | IL-1beta | |
| Secondary CD8 | 52.1 | KU-812 (Basophil) rest | 25.5 |
| lymphocyte act | | | |
| CD4 lymphocyte none | 21.0 | KU-812 (Basophil) | 68.8 |
| | | PMA/ionomycin | |
| 2ry Th1/Th2/Tr1_anti- | 41.8 | CCD1106 | 17.4 |
| CD95 CH11 | | (Keratinocytes) none | |
| LAK cells rest | 27.9 | CCD1106 | 36.6 |
| | | (Keratinocytes) | |
| | | TNF alpha + IL-1beta | |
| LAK cells IL-2 | 58.6 | Liver cirrhosis | 57.8 |
| LAK cells IL-2 + IL-12 | 61.1 | Lupus kidney | 24.3 |
| LAK cells IL-2 + IFN | 85.3 | NCI-H292 none | 20.0 |
| gamma | | | |
| LAK cells IL-2 + IL-18 | 61.1 | NCI-H292 IL-4 | 21.6 |
| LAK cells | 49.3 | NCI-H292 IL-9 | 24.5 |
| PMA/ionomycin | | | |
| NK Cells IL-2 rest | 43.2 | NCI-H292 IL-13 | 24.8 |
| Two Way MLR 3 day | 50.0 | NCI-H292 IFN gamma | 14.8 |
| Two Way MLR 5 day | 39.5 | HPAEC none | 9.5 |
| Two Way MLR 7 day | 37.1 | HPAEC TNF alpha + IL- | 25.9 |
| | | 1beta | |
| PBMC rest | 15.1 | Lung fibroblast none | 19.6 |
| PBMC PWM | 44.8 | Lung fibroblast TNF | 21.3 |
| | | alpha + IL-1beta | |
| PBMC PHA-L | 14.8 | Lung fibroblast IL-4 | 18.8 |
| Ramos (B cell) none | 75.3 | Lung fibroblast IL-9 | 17.8 |
| Ramos (B cell) | 17.8 | Lung fibroblast IL-13 | 21.9 |
| ionomycin | | | |
| B lymphocytes PWM | 33.4 | Lung fibroblast IFN | 21.3 |
| | | gamma | |
| B lymphocytes CD40L | 42.3 | Dermal fibroblast | 46.7 |
| and IL-4 | | CCD1070 rest | |
| EOL-1 dbcAMP | 25.3 | Dermal fibroblast | 69.3 |
| | | CCD1070 TNF alpha | |
| EOL-1 dbcAMP | 18.9 | Dermal fibroblast | 20.3 |
| PMA/ionomycin | | CCD1070 IL-1beta | |
| Dendritic cells none | 10.7 | Dermal fibroblast IFN | 17.4 |
| | | gamma | |
| Dendritic cells LPS | 13.6 | Dermal fibroblast IL-4 | 21.8 |
| Dendritic cells anti- | 12.2 | IBD Colitis 2 | 7.1 |
| CD40 | | | |
| Monocytes rest | 10.0 | IBD Crohn's | 5.5 |
| Monocytes LPS | 25.3 | Colon | 53.6 |
| Macrophages rest | 29.3 | Lung | 5.6 |
| Macrophages LPS | 7.2 | Thymus | 27.5 |
| HUVEC none | 24.1 | Kidney | 51.4 |
| HUVEC starved | 33.9 | | |

CNS_neurodegeneration_v1.0 Summary: Ag3509 This panel confirms the expression of this gene at low levels in the brain in an independent group of individuals. However, no differential expression of this gene was detected between Alzheimer's diseased postmortem brains and those of non-demented controls in this experiment.

General_screening_panel_v1.4 Summary: Ag3509 Highest expression of the CG59704-01 gene is detected in a sample derived from a lung cancer cell line (CT=31.69). Thus, expression of this gene can be used in distinguishing this sample from other samples in this panel. Furthermore, moderate expression of this gene is associated with cell lines derived from pancreatic, brain, colon, gastric, renal, lung, breast and ovarian cancers. Therefore, therapeutic modulation of the activity of this gene or its protein product, through the use of small molecule drugs, or antibodies, might be beneficial in the treatment of these cancers.

Panel 4D Summary: Ag3509 Expression of the CG59704-01 gene is stimulated in T cells, LAK cells and B cells, with highest expression in primary activated Tr1 cells (CT=32). Therefore, therapeutics designed with the protein encoded for by this transcript could be important in regulating T and B cell function and treating T cell/B cell mediated diseases such as asthma, arthritis, psoriasis, IBD, allergies, hypersensitivity reactions, microbial and viral infections systemic lupus erythematosus, multiple sclerosis, chronic obstructive pulmonary disease and systemic lupus erythematosus.

Furthermore, expression of this gene is decreased in colon samples from patients with IBD colitis and Crohn's disease relative to normal colon. Therefore, therapeutic modulation of the activity of this gene product may be useful in the treatment of inflammatory bowel disease.

Panel 5 Islet Summary: Ag3509 Expression of this gene is low/undetectable (CTs>35) across all of the samples on this panel (data not shown).

CA. CG59628-01: Short-Chain Dehydrogenase Like Homo Sapiens

Expression of gene CG59628-01 was assessed using the primer-probe set Ag3500, described in Table CAA. Results of the RTQ-PCR runs are shown in Tables CAB and CAC.

TABLE CCA

Probe Name Ag3500

| | | | Start | SEQ ID |
|---|---|---|---|---|
| Primers | Sequences | Length | Position | NO: |
| Forward | 5′-gcagatgtggtgatgagtatga-3′ | 22 | 1159 | 638 |
| Probe | TET-5′-tcaggtaaactaaaaccaacaatggca-3′-TAMRA | 27 | 1207 | 639 |
| Reverse | 5′-atcttcaatttccctgacatga-3′ | 22 | 1235 | 640 |

TABLE CAB
General_screening_panel_v1.4

| | Rel. Exp. | | Rel. Exp. |
|---|---|---|---|
| | (%) Ag3500, | | (%) Ag3500, |
| | Run | | Run |
| Tissue Name | 217131378 | Tissue Name | 217131378 |
| Adipose | 25.0 | Renal ca. TK-10 | 82.4 |
| Melanoma* | 19.3 | Bladder | 15.6 |
| Hs688(A).T | | | |
| Melanoma* | 18.3 | Gastric ca. (liver met.) | 36.3 |
| Hs688(B).T | | NCI-N87 | |
| Melanoma* M14 | 33.9 | Gastric ca. KATO III | 51.1 |
| Melanoma* | 36.3 | Colon ca. SW-948 | 17.8 |
| LOXIMVI | | | |
| Melanoma* SK- | 61.1 | Colon ca. SW480 | 66.4 |
| MEL-5 | | | |
| Squamous cell | 17.0 | Colon ca.* (SW480 | 36.1 |
| carcinoma SCC-4 | | met) SW620 | |
| Testis Pool | 11.4 | Colon ca. HT29 | 31.6 |
| Prostate ca.* (bone | 35.1 | Colon ca. HCT-116 | 46.0 |
| met) PC-3 | | | |
| Prostate Pool | 3.9 | Colon ca. CaCo-2 | 58.6 |
| Placenta | 1.2 | Colon cancer tissue | 24.1 |
| Uterus Pool | 1.9 | Colon ca. SW1116 | 6.7 |
| Ovarian ca. | 12.2 | Colon ca. Colo-205 | 12.1 |
| OVCAR-3 | | | |
| Ovarian ca. SK- | 55.9 | Colon ca. SW-48 | 25.0 |
| OV-3 | | | |
| Ovarian ca. | 7.2 | Colon Pool | 16.2 |
| OVCAR-4 | | | |
| Ovarian ca. | 47.6 | Small Intestine Pool | 9.9 |
| OVCAR-5 | | | |
| Ovarian ca. | 23.8 | Stomach Pool | 6.2 |
| IGROV-1 | | | |
| Ovarian ca. | 16.5 | Bone Marrow Pool | 4.6 |
| OVCAR-8 | | | |
| Ovary | 31.2 | Fetal Heart | 27.0 |
| Breast ca. MCF-7 | 9.7 | Heart Pool | 12.9 |
| Breast ca. MDA- | 33.2 | Lymph Node Pool | 13.1 |
| MB-231 | | | |
| Breast ca. BT 549 | 42.0 | Fetal Skeletal Muscle | 13.7 |
| Breast ca. T47D | 90.8 | Skeletal Muscle Pool | 40.9 |
| Breast ca. MDA-N | 18.9 | Spleen Pool | 11.6 |
| Breast Pool | 14.1 | Thymus Pool | 13.4 |
| Trachea | 7.7 | CNS cancer | 27.0 |
| | | (glio/astro) U87-MG | |
| Lung | 8.1 | CNS cancer | 39.0 |
| | | (glio/astro) U-118-MG | |
| Fetal Lung | 27.7 | CNS cancer | 20.0 |
| | | (neuro; met) SK-N-AS | |
| Lung ca. NCI-N417 | 4.4 | CNS cancer (astro) | 15.0 |
| | | SF-539 | |
| Lung ca. LX-1 | 30.1 | CNS cancer (astro) | 100.0 |
| | | SNB-75 | |
| Lung ca. NCI-H146 | 1.7 | CNS cancer (glio) | 22.5 |
| | | SNB-19 | |
| Lung ca. SHP-77 | 27.4 | CNS cancer (glio) SF- | 20.6 |
| | | 295 | |
| Lung ca. A549 | 24.5 | Brain (Amygdala) | 4.5 |
| | | Pool | |
| Lung ca. NCI-H526 | 5.6 | Brain (cerebellum) | 1.8 |
| Lung ca. NCI-H23 | 23.8 | Brain (fetal) | 2.0 |
| Lung ca. NCI-H460 | 22.2 | Brain (Hippocampus) | 9.2 |
| | | Pool | |
| Lung ca. HOP-62 | 14.4 | Cerebral Cortex Pool | 11.7 |
| Lung ca. NCI-H522 | 24.0 | Brain (Substantia | 7.7 |
| | | nigra) Pool | |
| Liver | 1.2 | Brain (Thalamus) Pool | 10.1 |
| Fetal Liver | 28.7 | Brain (whole) | 2.8 |
| Liver ca. HepG2 | 36.6 | Spinal Cord Pool | 17.2 |
| Kidney Pool | 10.8 | Adrenal Gland | 18.6 |
| Fetal Kidney | 13.6 | Pituitary gland Pool | 2.4 |
| Renal ca. 786-0 | 44.1 | Salivary Gland | 1.7 |
| Renal ca. A498 | 13.7 | Thyroid (female) | 11.3 |
| Renal ca. ACHN | 19.1 | Pancreatic ca. | 34.9 |
| | | CAPAN2 | |
| Renal ca. UO-31 | 37.4 | Pancreas Pool | 22.8 |

TABLE CAC
Panel 4D

| | Rel. Exp. (%) | | Rel. Exp. (%) |
|---|---|---|---|
| | Ag3500, Run | | Ag3500, Run |
| Tissue Name | 166441942 | Tissue Name | 166441942 |
| Secondary Th1 act | 6.0 | HUVEC IL-1beta | 6.8 |
| Secondary Th2 act | 10.1 | HUVEC IFN gamma | 17.1 |
| Secondary Tr1 act | 14.0 | HUVEC TNF alpha + | 5.6 |
| | | IFN gamma | |
| Secondary Th1 rest | 4.1 | HUVEC TNF alpha + | 6.2 |
| | | IL4 | |
| Secondary Th2 rest | 2.4 | HUVEC IL-11 | 7.2 |
| Secondary Tr1 rest | 3.1 | Lung Microvascular EC | 6.0 |
| | | none | |
| Primary Th1 act | 1.7 | Lung Microvascular EC | 4.5 |
| | | TNF alpha + IL-1beta | |
| Primary Th2 act | 6.4 | Microvascular Dermal | 20.0 |
| | | EC none | |
| Primary Tr1 act | 10.5 | Microsvasular Dermal | 5.9 |
| | | EC TNF alpha + IL-1beta | |
| Primary Th1 rest | 20.3 | Bronchial epithelium | 6.8 |
| | | TNF alpha + IL1beta | |
| Primary Th2 rest | 9.8 | Small airway epithelium | 11.2 |
| | | none | |
| Primary Tr1 rest | 18.0 | Small airway epithelium | 50.7 |
| | | TNF alpha + IL-1beta | |
| CD45RA CD4 | 11.5 | Coronery artery SMC rest | 6.8 |
| lymphocyte act | | | |
| CD45RO CD4 | 8.5 | Coronery artery SMC | 5.7 |
| lymphocyte act | | TNF alpha + IL-1beta | |
| CD8 lymphocyte act | 13.2 | Astrocytes rest | 17.9 |
| Secondary CD8 | 6.9 | Astrocytes TNF alpha + | 26.2 |
| lymphocyte rest | | IL-1beta | |
| Secondary CD8 | 6.2 | KU-812 (Basophil) rest | 13.2 |
| lymphocyte act | | | |
| CD4 lymphocyte none | 2.1 | KU-812 (Basophil) | 23.0 |
| | | PMA/ionomycin | |
| 2ry Th1/Th2/Tr1_anti- | 3.2 | CCD1106 | 14.5 |
| CD95 CH11 | | (Keratinocytes) none | |
| LAK cells rest | 2.5 | CCD1106 | 68.8 |
| | | (Keratinocytes) | |
| | | TNF alpha + IL-1beta | |
| LAK cells IL-2 | 12.2 | Liver cirrhosis | 20.4 |
| LAK cells IL-2 + IL-12 | 9.8 | Lupus kidney | 13.5 |
| LAK cells IL-2 + IFN | 11.7 | NCI-H292 none | 47.0 |
| gamma | | | |
| LAK cells IL-2 + IL-18 | 8.0 | NCI-H292 IL-4 | 21.8 |
| LAK cells | 2.0 | NCI-H292 IL-9 | 29.5 |
| PMA/ionomycin | | | |
| NK Cells IL-2 rest | 6.3 | NCI-H292 IL-13 | 13.3 |
| Two Way MLR 3 day | 6.4 | NCI-H292 IFN gamma | 12.3 |
| Two Way MLR 5 day | 12.3 | HPAEC none | 10.5 |
| Two Way MLR 7 day | 6.3 | HPAEC TNF alpha + IL- | 7.3 |
| | | 1beta | |
| PBMC rest | 3.3 | Lung fibroblast none | 10.1 |
| PBMC PWM | 11.6 | Lung fibroblast TNF | 15.1 |
| | | alpha + IL-1beta | |
| PBMC PHA-L | 5.7 | Lung fibroblast IL-4 | 9.1 |
| Ramos (B cell) none | 6.1 | Lung fibroblast IL-9 | 9.3 |
| Ramos (B cell) | 8.3 | Lung fibroblast IL-13 | 8.1 |
| ionomycin | | | |
| B lymphocytes PWM | 27.4 | Lung fibroblast IFN | 24.5 |
| | | gamma | |
| B lymphocytes CD40L | 15.0 | Dermal fibroblast | 39.8 |
| and IL-4 | | CCD1070 rest | |
| EOL-1 dbcAMP | 20.7 | Dermal fibroblast | 47.3 |
| | | CCD1070 TNF alpha | |
| EOL-1 dbcAMP | 4.7 | Dermal fibroblast | 12.4 |
| PMA/ionomycin | | CCD1070 IL-1beta | |
| Dendritic cells none | 6.9 | Dermal fibroblast IFN | 11.4 |
| | | gamma | |
| Dendritic cells LPS | 11.6 | Dermal fibroblast IL-4 | 48.3 |
| Dendritic cells anti- | 3.8 | IBD Colitis 2 | 2.2 |
| CD40 | | | |
| Monocytes rest | 6.0 | IBD Crohn's | 8.4 |
| Monocytes LPS | 2.3 | Colon | 100.0 |
| Macrophages rest | 12.2 | Lung | 14.0 |
| Macrophages LPS | 5.4 | Thymus | 73.2 |
| HUVEC none | 19.2 | Kidney | 25.0 |
| HUVEC starved | 31.9 | | |

CNS_neurodegeneration_v1.0 Summary: Ag3500 Results from one experiment with the CG59628-01 gene are not included. The amp plot indicates that there were experimental difficulties with this run.

General_screening_panel_v1.4 Summary: Ag3500 Highest expression of the CG59628-01 gene is detected in a sample derived from a CNS cancer cell line (CT=31.1). Therefore, expression of this gene may be used to distinguish this sample from the other samples on this panel. In addition, significant expression of this gene is associated with samples derived from colon, ovarian, breast, renal, lung, melanoma, and brain cancer cell lines. Therefore, therapeutic modulation of the activity of this gene or its protein product might be beneficial in the treatment of these cancers.

Among tissues with metabolic function, this gene is expressed at low but significant levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This molecule is also expressed at low levels in the CNS, including the hippocampus, thalamus, substantia nigra and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3500 Highest expression of the CG59628-01 gene is detected in colon (CT=30.3). Therefore, expression of this gene may be used to distinguish colon from the other tissues on this panel. Furthermore, expression of this gene is decreased in colon samples from patients with IBD colitis and Crohn's disease relative to normal colon. Therefore, therapeutic modulation of the activity of the GPCR encoded by this gene may be useful in the treatment of inflammatory bowel disease.

CB. CG59671-02: Acetyl-Coenzyme A Synthetase

Expression of gene CG59671-02 was assessed using the primer-probe sets Ag3506 and Ag3581, described in Tables CBA and CBB. Results of the RTQ-PCR runs are shown in Tables CBC, CBD, CBE and CBF.

TABLE CBA

Probe Name Ag3506

| | | | | SEQ ID |
|---|---|---|---|---|
| Primers | Sequences | Length | Start Position | NO: |
| Forward | 5′-aggacacagctacgtggtgtat-3′ | 22 | 1072 | 641 |
| Probe | TET-5′-cctctctgcaatggtgccaccag-3′-TAMRA | 23 | 1097 | 642 |
| Reverse | 5′-gataaactggggtgctctcaa-3′ | 21 | 1128 | 643 |

TABLE CBB

Probe Name Ag3581

| | | | Start | SEQ ID |
|---|---|---|---|---|
| Primers | Sequences | Length | Position | NO: |
| Forward | 5′-aggacacagctacgtggtgtat-3′ | 22 | 1072 | 644 |
| Probe | TET-5′-cctctctgcaatggtgccaccag-3′-TAMRA | 23 | 1097 | 645 |
| Reverse | 5′-gataaactggggtgctctcaa-3′ | 21 | 1128 | 646t |

TABLE CBC
CNS_neurodegeneration_v1.0

| | Rel. Exp. (%) | Rel. Exp. (%) | | Rel. Exp. (%) | Rel. Exp. (%) |
|---|---|---|---|---|---|
| Tissue | Ag3506, Run | Ag3581, Run | Tissue | Ag3506, Run | Ag3581, Run |
| Name | 210497900 | 210643840 | Name | 210497900 | 210643840 |
| AD 1 Hippo | 30.1 | 36.9 | Control | 15.8 | 21.5 |
| | | | (Path) 3 | | |
| | | | Temporal | | |
| | | | Ctx | | |
| AD 2 Hippo | 87.7 | 89.5 | Control | 42.6 | 53.6 |
| | | | (Path) 4 | | |
| | | | Temporal | | |
| | | | Ctx | | |
| AD 3 Hippo | 12.5 | 12.5 | AD 1 | 35.6 | 31.4 |
| | | | Occipital | | |
| | | | Ctx | | |
| AD 4 Hippo | 29.1 | 38.4 | AD 2 | 0.0 | 0.0 |
| | | | Occipital | | |
| | | | Ctx | | |
| | | | (Missing) | | |
| AD 5 Hippo | 55.5 | 69.3 | AD 3 | 17.8 | 18.7 |
| | | | Occipital | | |
| | | | Ctx | | |
| AD 6 Hippo | 24.1 | 33.4 | AD 4 | 47.0 | 38.4 |
| | | | Occipital | | |
| | | | Ctx | | |
| Control 2 | 42.3 | 56.3 | AD 5 | 42.9 | 26.1 |
| Hippo | | | Occipital | | |
| | | | Ctx | | |
| Control 4 | 42.6 | 60.3 | AD 6 | 24.3 | 51.1 |
| Hippo | | | Occipital | | |
| | | | Ctx | | |
| Control | 19.8 | 23.0 | Control 1 | 20.3 | 16.2 |
| (Path) 3 | | | Occipital | | |
| Hippo | | | Ctx | | |
| AD 1 | 63.7 | 48.3 | Control 2 | 71.2 | 63.7 |
| Temporal | | | Occipital | | |
| Ctx | | | Ctx | | |
| AD 2 | 60.7 | 87.7 | Control 3 | 35.4 | 41.5 |
| Temporal | | | Occipital | | |
| Ctx | | | Ctx | | |
| AD 3 | 15.9 | 15.6 | Control 4 | 20.0 | 27.9 |
| Temporal | | | Occipital | | |
| Ctx | | | Ctx | | |
| AD 4 | 47.0 | 57.0 | Control | 81.2 | 100.0 |
| Temporal | | | (Path) 1 | | |
| Ctx | | | Occipital | | |
| | | | Ctx | | |
| AD 5 Inf | 68.3 | 72.2 | Control | 18.9 | 26.4 |
| Temporal | | | (Path) 2 | | |
| Ctx | | | Occipital | | |
| | | | Ctx | | |
| AD 5 Sup | 65.5 | 57.8 | Control | 15.7 | 10.1 |
| Temporal | | | (Path) 3 | | |
| Ctx | | | Occipital | | |
| | | | Ctx | | |
| AD 6 Inf | 29.5 | 35.6 | Control | 24.8 | 26.8 |
| Temporal | | | (Path) 4 | | |
| Ctx | | | Occipital | | |
| | | | Ctx | | |
| AD 6 Sup | 31.2 | 31.9 | Control 1 | 21.9 | 33.7 |
| Temporal | | | Parietal Ctx | | |
| Ctx | | | | | |
| Control 1 | 22.5 | 29.3 | Control 2 | 51.8 | 79.0 |
| Temporal | | | Parietal Ctx | | |
| Ctx | | | | | |
| Control 2 | 55.1 | 58.6 | Control 3 | 31.4 | 38.4 |
| Temporal | | | Parietal Ctx | | |
| Ctx | | | | | |
| Control 3 | 26.4 | 34.6 | Control | 100.0 | 93.3 |
| Temporal | | | (Path) 1 | | |
| Ctx | | | Parietal Ctx | | |
| Control 3 | 35.6 | 35.8 | Control | 51.1 | 57.4 |
| Temporal | | | (Path) 2 | | |
| Ctx | | | Parietal Ctx | | |
| Control | 62.9 | 73.7 | Control | 17.1 | 17.2 |
| (Path) 1 | | | (Path) 3 | | |
| Temporal | | | Parietal Ctx | | |
| Ctx | | | | | |
| Control | 54.0 | 55.1 | Control | 54.3 | 55.9 |
| (Path) 2 | | | (Path) 4 | | |
| Temporal | | | Parietal Ctx | | |
| Ctx | | | | | |

TABLE CBD
General_screening_panel_v1.4

| | Rel. Exp. (%) | Rel. Exp. (%) | | Rel. Exp. (%) | Rel. Exp. (%) |
|---|---|---|---|---|---|
| | Ag3506, Run | Ag3581, Run | | Ag3506, Run | Ag3581, Run |
| Tissue Name | 217236187 | 217423588 | Tissue Name | 217236187 | 217423588 |
| Adipose | 3.6 | 3.5 | Renal ca. TK-10 | 4.1 | 3.4 |
| Melanoma* | 1.8 | 1.0 | Bladder | 16.5 | 12.2 |
| Hs688(A).T | | | | | |
| Melanoma* | 0.6 | 0.6 | Gastric ca. (liver | 21.5 | 17.3 |
| Hs688(B).T | | | met.) NCI-N87 | | |
| Melanoma* | 100.0 | 30.4 | Gastric ca. | 63.3 | 53.2 |
| M14 | | | KATO III | | |
| Melanoma* | 0.0 | 0.0 | Colon ca. SW- | 8.0 | 5.0 |
| LOXIMVI | | | 948 | | |
| Melanoma* | 7.5 | 100.0 | Colon ca. | 20.7 | 16.6 |
| SK-MEL-5 | | | SW480 | | |
| Squamous | 10.9 | 6.3 | Colon ca.* | 0.0 | 0.1 |
| cell | | | (SW480 met) | | |
| carcinoma | | | SW620 | | |
| SCC-4 | | | | | |
| Testis Pool | 11.9 | 9.8 | Colon ca. HT29 | 24.7 | 15.7 |
| Prostate ca.* | 2.0 | 2.3 | Colon ca. HCT- | 0.0 | 0.0 |
| (bone met) | | | 116 | | |
| PC-3 | | | | | |
| Prostate Pool | 13.0 | 7.5 | Colon ca. CaCo-2 | 60.3 | 40.1 |
| Placenta | 87.1 | 50.3 | Colon cancer | 30.8 | 18.8 |
| | | | tissue | | |
| Uterus Pool | 7.0 | 4.5 | Colon ca. | 5.3 | 2.4 |
| | | | SW1116 | | |
| Ovarian ca. | 28.5 | 14.4 | Colon ca. Colo- | 1.9 | 1.9 |
| OVCAR-3 | | | 205 | | |
| Ovarian ca. | 7.7 | 4.2 | Colon ca. SW-48 | 33.2 | 16.8 |
| SK-OV-3 | | | | | |
| Ovarian ca. | 2.6 | 1.0 | Colon Pool | 14.3 | 15.1 |
| OVCAR-4 | | | | | |
| Ovarian ca. | 28.3 | 14.1 | Small Intestine | 15.0 | 7.4 |
| OVCAR-5 | | | Pool | | |
| Ovarian ca. | 0.5 | 0.2 | Stomach Pool | 8.3 | 4.8 |
| IGROV-1 | | | | | |
| Ovarian ca. | 1.4 | 1.1 | Bone Marrow | 7.3 | 4.7 |
| OVCAR-8 | | | Pool | | |
| Ovary | 7.9 | 7.3 | Fetal Heart | 16.0 | 12.8 |
| Breast ca. | 3.8 | 2.1 | Heart Pool | 13.1 | 10.1 |
| MCF-7 | | | | | |
| Breast ca. | 8.2 | 6.0 | Lymph Node | 16.3 | 9.2 |
| MDA-MB- | | | Pool | | |
| 231 | | | | | |
| Breast ca. BT | 1.6 | 1.2 | Fetal Skeletal | 6.9 | 3.7 |
| 549 | | | Muscle | | |
| Breast ca. | 62.9 | 31.2 | Skeletal Muscle | 18.9 | 15.1 |
| T47D | | | Pool | | |
| Breast ca. | 29.7 | 15.4 | Spleen Pool | 8.8 | 6.9 |
| MDA-N | | | | | |
| Breast Pool | 12.5 | 8.8 | Thymus Pool | 19.6 | 11.6 |
| Trachea | 12.4 | 8.2 | CNS cancer | 0.6 | 0.3 |
| | | | (glio/astro) U87- | | |
| | | | MG | | |
| Lung | 1.7 | 0.8 | CNS cancer | 0.4 | 0.2 |
| | | | (glio/astro) U- | | |
| | | | 118-MG | | |
| Fetal Lung | 29.7 | 18.0 | CNS cancer | 3.4 | 2.2 |
| | | | (neuro; met) SK- | | |
| | | | N-AS | | |
| Lung ca. | 0.1 | 0.0 | CNS cancer | 0.1 | 0.0 |
| NCI-N417 | | | (astro) SF-539 | | |
| Lung ca. LX-1 | 0.1 | 0.1 | CNS cancer | 10.4 | 2.1 |
| | | | (astro) SNB-75 | | |
| Lung ca. | 0.3 | 0.1 | CNS cancer | 1.0 | 0.2 |
| NCI-H146 | | | (glio) SNB-19 | | |
| Lung ca. | 7.8 | 5.0 | CNS cancer | 1.3 | 0.6 |
| SHP-77 | | | (glio) SF-295 | | |
| Lung ca. | 7.8 | 5.1 | Brain | 10.3 | 5.9 |
| A549 | | | (Amygdala) Pool | | |
| Lung ca. | 10.2 | 2.5 | Brain | 32.3 | 21.0 |
| NCI-H526 | | | (cerebellum) | | |
| Lung ca. | 3.4 | 3.2 | Brain (fetal) | 9.7 | 4.7 |
| NCI-H23 | | | | | |
| Lung ca. | 1.9 | 0.8 | Brain | 15.5 | 10.6 |
| NCI-H460 | | | (Hippocampus) | | |
| | | | Pool | | |
| Lung ca. | 0.9 | 0.6 | Cerebral Cortex | 20.7 | 14.5 |
| HOP-62 | | | Pool | | |
| Lung ca. | 0.1 | 0.1 | Brain (Substantia | 18.8 | 9.9 |
| NCI-H522 | | | nigra) Pool | | |
| Liver | 0.8 | 0.5 | Brain | 15.9 | 10.4 |
| | | | (Thalamus) Pool | | |
| Fetal Liver | 37.1 | 22.4 | Brain (whole) | 20.3 | 14.3 |
| Liver ca. | 1.8 | 0.8 | Spinal Cord Pool | 14.8 | 9.0 |
| HepG2 | | | | | |
| Kidney Pool | 18.7 | 14.9 | Adrenal Gland | 5.6 | 3.8 |
| Fetal Kidney | 6.2 | 6.0 | Pituitary gland | 1.6 | 1.0 |
| | | | Pool | | |
| Renal ca. | 0.3 | 0.1 | Salivary Gland | 15.3 | 8.6 |
| 786-0 | | | | | |
| Renal ca. | 1.8 | 1.1 | Thyroid (female) | 11.0 | 11.4 |
| A498 | | | | | |
| Renal ca. | 4.3 | 1.4 | Pancreatic ca. | 0.3 | 0.2 |
| ACHN | | | CAPAN2 | | |
| Renal ca. | 9.3 | 2.5 | Pancreas Pool | 16.7 | 16.5 |
| UO-31 | | | | | |

TABLE CBE
Panel 4.1D

| | Rel. Exp. (%) | | Rel. Exp. (%) |
|---|---|---|---|
| | Ag3581, Run | | Ag3581, Run |
| Tissue Name | 169910402 | Tissue Name | 169910402 |
| Secondary Th1 act | 36.6 | HUVEC IL-1beta | 4.0 |
| Secondary Th2 act | 38.2 | HUVEC IFN gamma | 19.5 |
| Secondary Tr1 act | 45.1 | HUVEC TNF alpha + | 5.3 |
| | | IFN gamma | |
| Secondary Th1 rest | 72.2 | HUVEC TNF alpha + | 2.6 |
| | | IL4 | |
| Secondary Th2 rest | 58.2 | HUVEC IL-11 | 11.5 |
| Secondary Tr1 rest | 79.0 | Lung Microvascular EC | 27.9 |
| | | none | |
| Primary Th1 act | 23.2 | Lung Microvascular EC | 28.1 |
| | | TNF alpha + IL-1beta | |
| Primary Th2 act | 48.6 | Microvascular Dermal | 7.9 |
| | | EC none | |
| Primary Tr1 act | 38.4 | Microsvasular Dermal | 10.6 |
| | | EC TNF alpha + IL-1beta | |
| Primary Th1 rest | 90.8 | Bronchial epithelium | 6.9 |
| | | TNF alpha + IL1beta | |
| Primary Th2 rest | 99.3 | Small airway epithelium | 1.7 |
| | | none | |
| Primary Tr1 rest | 90.8 | Small airway epithelium | 3.2 |
| | | TNF alpha + IL-1beta | |
| CD45RA CD4 | 15.2 | Coronery artery SMC rest | 0.3 |
| lymphocyte act | | | |
| CD45RO CD4 | 45.1 | Coronery artery SMC | 0.3 |
| lymphocyte act | | TNF alpha + IL-1beta | |
| CD8 lymphocyte act | 36.3 | Astrocytes rest | 0.0 |
| Secondary CD8 | 27.0 | Astrocytes TNF alpha + | 0.0 |
| lymphocyte rest | | IL-1beta | |
| Secondary CD8 | 41.2 | KU-812 (Basophil) rest | 100.0 |
| lymphocyte act | | | |
| CD4 lymphocyte none | 31.0 | KU-812 (Basophil) | 25.2 |
| | | PMA/ionomycin | |
| 2ry Th1/Th2/Tr1_anti- | 77.4 | CCD1106 | 13.0 |
| CD95 CH11 | | (Keratinocytes) none | |
| LAK cells rest | 40.6 | CCD1106 | 19.3 |
| | | (Keratinocytes) | |
| | | TNF alpha + IL-1beta | |
| LAK cells IL-2 | 66.0 | Liver cirrhosis | 8.9 |
| LAK cells IL-2 + IL-12 | 28.3 | NCI-H292 none | 0.5 |
| LAK cells IL-2 + IFN | 34.9 | NCI-H292 IL-4 | 0.8 |
| gamma | | | |
| LAK cells IL-2 + IL-18 | 32.1 | NCI-H292 IL-9 | 0.8 |
| LAK cells | 9.3 | NCI-H292 IL-13 | 1.2 |
| PMA/ionomycin | | | |
| NK Cells IL-2 rest | 82.4 | NCI-H292 IFN gamma | 3.0 |
| Two Way MLR 3 day | 59.9 | HPAEC none | 8.6 |
| Two Way MLR 5 day | 28.7 | HPAEC TNF alpha + IL- | 7.7 |
| | | 1beta | |
| Two Way MLR 7 day | 27.4 | Lung fibroblast none | 1.9 |
| PBMC rest | 45.1 | Lung fibroblast TNF | 0.1 |
| | | alpha + IL-1beta | |
| PBMC PWM | 19.5 | Lung fibroblast IL-4 | 1.2 |
| PBMC PHA-L | 28.1 | Lung fibroblast IL-9 | 0.3 |
| Ramos (B cell) none | 47.0 | Lung fibroblast IL-13 | 0.6 |
| Ramos (B cell) | 37.4 | Lung fibroblast IFN | 2.6 |
| ionomycin | | gamma | |
| B lymphocytes PWM | 16.4 | Dermal fibroblast | 3.6 |
| | | CCD1070 rest | |
| B lymphocytes CD40L | 48.6 | Dermal fibroblast | 73.7 |
| and IL-4 | | CCD1070 TNF alpha | |
| EOL-1 dbcAMP | 0.3 | Dermal fibroblast | 3.5 |
| | | CCD1070 IL-1beta | |
| EOL-1 dbcAMP | 0.0 | Dermal fibroblast IFN | 8.3 |
| PMA/ionomycin | | gamma | |
| Dendritic cells none | 24.0 | Dermal fibroblast IL-4 | 6.1 |
| Dendritic cells LPS | 6.5 | Dermal Fibroblasts rest | 23.0 |
| Dendritic cells anti- | 24.1 | Neutrophils TNFa + LPS | 2.1 |
| CD40 | | | |
| Monocytes rest | 44.8 | Neutrophils rest | 8.7 |
| Monocytes LPS | 10.7 | Colon | 69.3 |
| Macrophages rest | 38.7 | Lung | 27.7 |
| Macrophages LPS | 9.5 | Thymus | 75.3 |
| HUVEC none | 2.9 | Kidney | 84.7 |
| HUVEC starved | 3.3 | | |

TABLE CBF
Panel 4D

| | Rel. Exp. (%) | | Rel. Exp. (%) |
|---|---|---|---|
| | Ag3506, Run | | Ag3506, Run |
| Tissue Name | 166407188 | Tissue Name | 166407188 |
| Secondary Th1 act | 8.4 | HUVEC IL-1beta | 0.4 |
| Secondary Th2 act | 7.2 | HUVEC IFN gamma | 6.7 |
| Secondary Tr1 act | 11.4 | HUVEC TNF alpha + | 1.1 |
| | | IFN gamma | |
| Secondary Th1 rest | 31.6 | HUVEC TNF alpha + | 1.0 |
| | | IL4 | |
| Secondary Th2 rest | 13.7 | HUVEC IL-11 | 2.3 |
| Secondary Tr1 rest | 22.5 | Lung Microvascular EC | 3.8 |
| | | none | |
| Primary Th1 act | 3.9 | Lung Microvascular EC | 4.0 |
| | | TNF alpha + IL-1beta | |
| Primary Th2 act | 14.6 | Microvascular Dermal | 3.4 |
| | | EC none | |
| Primary Tr1 act | 14.3 | Microsvasular Dermal | 2.8 |
| | | EC TNF alpha + IL-1beta | |
| Primary Th1 rest | 69.7 | Bronchial epithelium | 0.9 |
| | | TNF alpha + IL1beta | |
| Primary Th2 rest | 44.1 | Small airway epithelium | 0.2 |
| | | none | |
| Primary Tr1 rest | 28.9 | Small airway epithelium | 1.2 |
| | | TNF alpha + IL-1beta | |
| CD45RA CD4 | 3.2 | Coronery artery SMC rest | 0.0 |
| lymphocyte act | | | |
| CD45RO CD4 | 12.3 | Coronery artery SMC | 0.3 |
| lymphocyte act | | TNF alpha + IL-1beta | |
| CD8 lymphocyte act | 8.7 | Astrocytes rest | 0.1 |
| Secondary CD8 | 9.5 | Astrocytes TNF alpha + | 0.1 |
| lymphocyte rest | | IL-1beta | |
| Secondary CD8 | 12.0 | KU-812 (Basophil) rest | 18.3 |
| lymphocyte act | | | |
| CD4 lymphocyte none | 9.6 | KU-812 (Basophil) | 14.9 |
| | | PMA/ionomycin | |
| 2ry Th1/Th2/Tr1_anti- | 23.8 | CCD1106 | 2.0 |
| CD95 CH11 | | (Keratinocytes) none | |
| LAK cells rest | 6.8 | CCD1106 | 11.7 |
| | | (Keratinocytes) | |
| | | TNF alpha + IL-1beta | |
| LAK cells IL-2 | 21.2 | Liver cirrhosis | 5.4 |
| LAK cells IL-2 + IL-12 | 8.5 | Lupus kidney | 12.2 |
| LAK cells IL-2 + IFN | 12.2 | NCI-H292 none | 0.2 |
| gamma | | | |
| LAK cells IL-2 + IL-18 | 12.0 | NCI-H292 IL-4 | 0.2 |
| LAK cells | 1.8 | NCI-H292 IL-9 | 0.2 |
| PMA/ionomycin | | | |
| NK Cells IL-2 rest | 16.0 | NCI-H292 IL-13 | 0.0 |
| Two Way MLR 3 day | 16.6 | NCI-H292 IFN gamma | 0.1 |
| Two Way MLR 5 day | 7.2 | HPAEC none | 2.9 |
| Two Way MLR 7 day | 9.6 | HPAEC TNF alpha + IL- | 2.8 |
| | | 1beta | |
| PBMC rest | 8.5 | Lung fibroblast none | 0.5 |
| PBMC PWM | 3.5 | Lung fibroblast TNF | 0.6 |
| | | alpha + IL-1beta | |
| PBMC PHA-L | 2.8 | Lung fibroblast IL-4 | 0.1 |
| Ramos (B cell) none | 11.3 | Lung fibroblast IL-9 | 0.1 |
| Ramos (B cell) | 5.6 | Lung fibroblast IL-13 | 0.0 |
| ionomycin | | | |
| B lymphocytes PWM | 6.2 | Lung fibroblast IFN | 0.5 |
| | | gamma | |
| B lymphocytes CD40L | 12.2 | Dermal fibroblast | 1.1 |
| and IL-4 | | CCD1070 rest | |
| EOL-1 dbcAMP | 0.1 | Dermal fibroblast | 21.8 |
| | | CCD1070 TNF alpha | |
| EOL-1 dbcAMP | 0.0 | Dermal fibroblast | 1.0 |
| PMA/ionomycin | | CCD1070 IL-1beta | |
| Dendritic cells none | 4.3 | Dermal fibroblast IFN | 2.1 |
| | | gamma | |
| Dendritic cells LPS | 1.4 | Dermal fibroblast IL-4 | 1.8 |
| Dendritic cells anti- | 4.6 | IBD Colitis 2 | 2.6 |
| CD40 | | | |
| Monocytes rest | 11.9 | IBD Crohn's | 5.5 |
| Monocytes LPS | 1.7 | Colon | 100.0 |
| Macrophages rest | 9.3 | Lung | 5.9 |
| Macrophages LPS | 1.6 | Thymus | 37.9 |
| HUVEC none | 0.9 | Kidney | 24.7 |
| HUVEC starved | 1.7 | | |

CNS_neurodegeneration_v1.0 Summary: Ag3506/Ag3581 This panel confirms the expression of the CG59671-02 gene at significant levels in the brain in an independent group of individuals. However, no differential expression of this gene was detected between Alzheimer's diseased postmortem brains and those of non-demented controls in this experiment.

This gene is expressed at moderate levels throughout the CNS, including in amygdala, substantia nigra, thalamus, cerebellum, cerebral cortex, and spinal cord as observed in panel 1.4. Therefore, this gene may play a role in other central nervous system disorders such as, Parkinson's disease, epilepsy, multiple sclerosis, schizophrenia and depression

General_screening_panel_v1.4 Summary: Ag3506/Ag3581 Two experiments produce results that are in very good agreement. Highest expression of the CG59671-02 gene is observed in samples derived from melanoma cell lines (CTs=23-35). Thus, expression of this gene can be used in distinguishing these samples from other samples in the panel. In addition, significant levels of expression of this gene are also associated with colon cancer, ovarian cancer, breast cancer, and lung cancer cell lines. Therefore, therapeutic modulation of the activity of this gene or its protein product might be beneficial in the treatment of these cancers.

This gene is also expressed at low to moderate levels in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as obesity and diabetes.

This gene is also expressed at high to moderate levels in all regions of the CNS examined. Please see Panel CNS_neurodegeneration_v1.0 for discussion of utility of this gene in the central nervous system.

Panel 4.1D Summary: Ag3581 Highest expression of the CG59671-02 gene is observed in the resting KU-812 sample (CT=29.18). In addition, high expression of this gene is detected in colon, lung, thymus and kidney. Therefore, therapies designed with the protein encoded for by this gene could be important in the treatment of inflammatory or autoimmune diseases that affect the kidney, lung and kidney including, asthma, allergies, lupus and glomerulonephritis. Expression of this gene is decreased in colon samples from patients with IBD colitis and Crohn's disease relative to normal colon. Therefore, therapeutic modulation of the activity of the protein encoded by this gene may also be useful in the treatment of inflammatory bowel disease.

Expression of this gene appears to be down-regulated in activated primary and secondary Th1, Th2, and Tr1 cells. Also, TNF alpha stimulates the expression of this gene in resting dermal fibroblasts. Therefore, therapeutics designed with the protein encoded by this transcript could be important in regulating T cell function and treating diseases such as asthma, arthritis, psoriasis, IBD, and systemic lupus erythematosus.

Panel 4D Summary: Ag3506 Highest expression of CG59671-02 is observed colon sample (CT=27.3). Overall, the expression pattern using this probe is in excellent agreement with results in panel 4.1 D for Ag3581. Please see that panel for discussion of utility of this gene in inflammation.

CC. CG56870-01: NDR3

Expression of gene CG56870-01 was assessed using the primer-probe set Ag2075, described in Table CCA. Results of the RTQ-PCR runs are shown in Tables CCB, CCC, CCD and CCE. Please note that CG56870-02 represents a full-length physical clone of the CG56870-01 gene, validating the prediction of the gene sequence.

Table CCA. Probe Name Ag2075

TABLE CCA
Probe Name Ag2075

| | | | Start | SEQ ID |
|---|---|---|---|---|
| Primers | Sequences | Length | Position | NO: |
| Forward | 5′-catggatgaacttcaggatgtt-3′ | 22 | 70 | 647 |
| Probe | TET-5′-cagctcacagagatcaaaccacttct-3′-TAMRA | 26 | 92 | 648 |
| Reverse | 5′-tgacagtcaaagtcctggaagt-3′ | 22 | 141 | 649 |

TABLE CCB
Panel 1.3D

| | Rel. Exp. (%) | | Rel. Exp. (%) |
|---|---|---|---|
| | Ag2075, | | Ag2075, |
| Tissue Name | Run 152355202 | Tissue Name | Run 152355202 |
| Liver adenocarcinoma | 11.9 | Kidney (fetal) | 1.4 |
| Pancreas | 0.8 | Renal ca. 786-0 | 3.5 |
| Pancreatic ca. CAPAN2 | 1.6 | Renal ca. A498 | 12.9 |
| Adrenal gland | 2.4 | Renal ca. RXF 393 | 1.5 |
| Thyroid | 1.8 | Renal ca. ACHN | 1.7 |
| Salivary gland | 1.2 | Renal ca. UO-31 | 6.8 |
| Pituitary gland | 2.4 | Renal ca. TK-10 | 2.8 |
| Brain (fetal) | 3.2 | Liver | 0.4 |
| Brain (whole) | 25.2 | Liver (fetal) | 0.8 |
| Brain (amygdala) | 14.1 | Liver ca. | 6.7 |
| | | (hepatoblast) HepG2 | |
| Brain (cerebellum) | 10.8 | Lung | 2.1 |
| Brain (hippocampus) | 39.8 | Lung (fetal) | 3.2 |
| Brain (substantia nigra) | 2.7 | Lung ca. (small cell) | 4.1 |
| | | LX-1 | |
| Brain (thalamus) | 12.4 | Lung ca. (small cell) | 3.8 |
| | | NCI-H69 | |
| Cerebral Cortex | 100.0 | Lung ca. (s.cell var.) | 3.8 |
| | | SHP-77 | |
| Spinal cord | 3.5 | Lung ca. (large | 0.6 |
| | | cell)NCI-H460 | |
| glio/astro U87-MG | 6.4 | Lung ca. (non-sm. | 3.0 |
| | | cell) A549 | |
| glio/astro U-118-MG | 10.4 | Lung ca. (non-s.cell) | 7.7 |
| | | NCI-H23 | |
| astrocytoma SW1783 | 5.1 | Lung ca. (non-s.cell) | 3.1 |
| | | HOP-62 | |
| neuro*; met SK-N-AS | 6.2 | Lung ca. (non-s.cl) | 6.3 |
| | | NCI-H522 | |
| astrocytoma SF-539 | 5.4 | Lung ca. (squam.) | 1.4 |
| | | SW 900 | |
| astrocytoma SNB-75 | 6.7 | Lung ca. (squam.) | 1.4 |
| | | NCI-H596 | |
| glioma SNB-19 | 3.1 | Mammary gland | 2.4 |
| glioma U251 | 2.0 | Breast ca.* (pl.ef) | 1.8 |
| | | MCF-7 | |
| glioma SF-295 | 3.4 | Breast ca.* (pl.ef) | 11.6 |
| | | MDA-MB-231 | |
| Heart (fetal) | 6.7 | Breast ca.* (pl.ef) | 9.0 |
| | | T47D | |
| Heart | 1.4 | Breast ca. BT-549 | 4.8 |
| Skeletal muscle (fetal) | 18.0 | Breast ca. MDA-N | 4.2 |
| Skeletal muscle | 0.7 | Ovary | 13.4 |
| Bone marrow | 0.9 | Ovarian ca. | 1.8 |
| | | OVCAR-3 | |
| Thymus | 0.9 | Ovarian ca. | 1.6 |
| | | OVCAR-4 | |
| Spleen | 3.7 | Ovarian ca. | 1.8 |
| | | OVCAR-5 | |
| Lymph node | 1.9 | Ovarian ca. | 4.9 |
| | | OVCAR-8 | |
| Colorectal | 3.3 | Ovarian ca. IGROV-1 | 1.6 |
| Stomach | 2.7 | Ovarian ca.* | 5.8 |
| | | (ascites) SK-OV-3 | |
| Small intestine | 2.5 | Uterus | 2.4 |
| Colon ca. SW480 | 10.4 | Placenta | 1.2 |
| Colon ca.* | 3.7 | Prostate | 6.0 |
| SW620(SW480 met) | | | |
| Colon ca. HT29 | 4.6 | Prostate ca.* (bone | 4.5 |
| | | met)PC-3 | |
| Colon ca. HCT-116 | 3.6 | Testis | 1.7 |
| Colon ca. CaCo-2 | 10.3 | Melanoma | 3.8 |
| | | Hs688(A).T | |
| Colon ca. | 3.2 | Melanoma* (met) | 4.4 |
| tissue(ODO3866) | | Hs688(B).T | |
| Colon ca. HCC-2998 | 2.6 | Melanoma UACC- | 1.4 |
| | | 62 | |
| Gastric ca.* (liver met) | 3.1 | Melanoma M14 | 1.8 |
| NCI-N87 | | | |
| Bladder | 0.8 | Melanoma LOX | 3.3 |
| | | IMVI | |
| Trachea | 2.0 | Melanoma* (met) | 2.4 |
| | | SK-MEL-5 | |
| Kidney | 1.3 | Adipose | 1.2 |

TABLE CCC
Panel 2.2

| | Rel. Exp. | | Rel. Exp. |
|---|---|---|---|
| | (%) Ag2075, | | (%) Ag2075, |
| | Run | | Run |
| Tissue Name | 174255357 | Tissue Name | 174255357 |
| Normal Colon | 27.7 | Kidney Margin | 46.3 |
| | | (OD04348) | |
| Colon cancer | 52.9 | Kidney malignant | 19.2 |
| (OD06064) | | cancer (OD06204B) | |
| Colon Margin | 30.1 | Kidney normal | 14.3 |
| (OD06064) | | adjacent tissue | |
| | | (OD06204E) | |
| Colon cancer | 5.5 | Kidney Cancer | 66.0 |
| (OD06159) | | (OD04450-01) | |
| Colon Margin | 21.0 | Kidney Margin | 19.8 |
| (OD06159) | | (OD04450-03) | |
| Colon cancer | 22.2 | Kidney Cancer | 3.0 |
| (OD06297-04) | | 8120613 | |
| Colon Margin | 41.8 | Kidney Margin | 11.0 |
| (OD06297-05) | | 8120614 | |
| CC Gr.2 ascend colon | 4.0 | Kidney Cancer | 6.8 |
| (ODO3921) | | 9010320 | |
| CC Margin | 6.7 | Kidney Margin | 9.7 |
| (OD03921) | | 9010321 | |
| Colon cancer metastasis | 11.1 | Kidney Cancer | 35.6 |
| (OD06104) | | 8120607 | |
| Lung Margin | 42.6 | Kidney Margin | 13.7 |
| (OD06104) | | 8120608 | |
| Colon mets to lung | 17.8 | Normal Uterus | 59.5 |
| (OD04451-01) | | | |
| Lung Margin | 9.3 | Uterine Cancer 064011 | 9.7 |
| (OD04451-02) | | | |
| Normal Prostate | 51.4 | Normal Thyroid | 8.8 |
| Prostate Cancer | 23.0 | Thyroid Cancer | 11.2 |
| (OD04410) | | 064010 | |
| Prostate Margin | 19.3 | Thyroid Cancer | 17.9 |
| (OD04410) | | A302152 | |
| Normal Ovary | 22.8 | Thyroid Margin | 5.5 |
| | | A302153 | |
| Ovarian cancer | 9.9 | Normal Breast | 33.2 |
| (OD06283-03) | | | |
| Ovarian Margin | 17.1 | Breast Cancer | 8.5 |
| (OD06283-07) | | (OD04566) | |
| Ovarian Cancer 064008 | 18.0 | Breast Cancer 1024 | 36.1 |
| Ovarian cancer | 6.6 | Breast Cancer | 18.4 |
| (OD06145) | | (OD04590-01) | |
| Ovarian Margin | 12.5 | Breast Cancer Mets | 31.9 |
| (OD06145) | | (OD04590-03) | |
| Ovarian cancer | 14.7 | Breast Cancer | 45.4 |
| (OD06455-03) | | Metastasis (OD0465505) | |
| Ovarian Margin | 21.8 | Breast Cancer 064006 | 11.5 |
| (OD06455-07) | | | |
| Normal Lung | 21.9 | Breast Cancer | 20.9 |
| Invasive poor diff. lung | 17.6 | 9100266 Breast | 35.1 |
| adeno (OD04945-01) | | Margin 9100265 | |
| Lung Margin | 12.2 | Breast Cancer | 9.7 |
| (ODO4945-03) | | A209073 | |
| Lung Malignant Cancer | 8.7 | Breast Margin | 22.2 |
| (OD03126) | | A2090734 | |
| Lung Margin | 7.4 | Breast cancer | 100.0 |
| (OD03126) | | (OD06083) | |
| Lung Cancer | 9.9 | Breast cancer node | 63.7 |
| (OD05014A) | | metastasis (OD06083) | |
| Lung Margin | 21.8 | Normal Liver | 9.9 |
| (OD05014B) | | | |
| Lung cancer | 5.1 | Liver Cancer 1026 | 5.6 |
| (OD06081) | | | |
| Lung Margin | 7.9 | Liver Cancer 1025 | 13.5 |
| (OD06081) | | | |
| Lung Cancer | 17.4 | Liver Cancer 6004-T | 4.8 |
| (OD04237-01) | | | |
| Lung Margin | 24.0 | Liver Tissue 6004-N | 9.3 |
| (OD04237-02) | | | |
| Ocular Melanoma | 9.7 | Liver Cancer 6005-T | 15.7 |
| Metastasis | | | |
| Ocular Melanoma | 4.6 | Liver Tissue 6005-N | 20.4 |
| Margin (Liver) | | | |
| Melanoma Metastasis | 19.8 | Liver Cancer 064003 | 10.7 |
| Melanoma Margin | 21.6 | Normal Bladder | 8.0 |
| (Lung) | | | |
| Normal Kidney | 11.0 | Bladder Cancer 1023 | 10.5 |
| Kidney Ca, Nuclear | 37.6 | Bladder Cancer | 17.3 |
| grade 2 (OD04338) | | A302173 | |
| Kidney Margin | 22.1 | Normal Stomach | 37.9 |
| (OD04338) | | | |
| Kidney Ca Nuclear | 21.6 | Gastric Cancer | 11.1 |
| grade 1/2 (OD04339) | | 9060397 | |
| Kidney Margin | 12.9 | Stomach Margin | 20.6 |
| (OD04339) | | 9060396 | |
| Kidney Ca, Clear | 6.5 | Gastric Cancer | 22.7 |
| cell type (OD04340) | | 9060395 | |
| Kidney Margin | 18.4 | Stomach Margin | 36.1 |
| (OD04340) | | 9060394 | |
| Kidney Ca, Nuclear | 12.9 | Gastric Cancer 064005 | 8.1 |
| grade 3 (OD04348) | | | |

TABLE CCD
Panel 3D

| | Rel. Exp. (%) | | Rel. Exp. (%) |
|---|---|---|---|
| | Ag2075, Run | | Ag2075, Run |
| Tissue Name | 164750734 | Tissue Name | 164750734 |
| Daoy-Medulloblastoma | 6.7 | Ca Ski-Cervical epidermoid | 50.3 |
| | | carcinoma (metastasis) | |
| TE671- | 9.0 | ES-2-Ovarian clear cell | 13.9 |
| Medulloblastoma | | carcinoma | |
| D283 Med- | 35.4 | Ramos-Stimulated with | 2.7 |
| Medulloblastoma | | PMA/ionomycin 6 h | |
| PFSK-1-Primitive | 15.4 | Ramos-Stimulated with | 3.3 |
| Neuroectodermal | | PMA/ionomycin 14 h | |
| XF-498-CNS | 4.4 | MEG-01-Chronic | 3.4 |
| | | myelogenous leukemia | |
| | | (megokaryoblast) | |
| SNB-78-Glioma | 23.3 | Raji-Burkitt's Lymphoma | 3.7 |
| SF-268-Glioblastoma | 13.6 | Daudi-Burkitt's lymphoma | 6.5 |
| T98G-Glioblastoma | 18.6 | U266-B-cell plasmacytoma | 8.4 |
| SK-N-SH- | 17.0 | CA46-Burkitt's lymphoma | 7.2 |
| Neuroblastoma | | | |
| (metastasis) | | | |
| SF-295-Glioblastoma | 8.4 | RL-non-Hodgkin's B-cell | 3.5 |
| | | lymphoma | |
| Cerebellum | 74.7 | JM1-pre-B-cell lymphoma | 3.1 |
| Cerebellum | 62.4 | Jurkat-T cell leukemia | 11.6 |
| NCI-H292- | 45.7 | TF-1-Erythroleukemia | 13.4 |
| Mucoepidermoid lung | | | |
| carcinoma | | | |
| DMS-114-Small cell | 10.9 | HUT 78-T-cell lymphoma | 10.0 |
| lung cancer | | | |
| DMS-79-Small cell | 100.0 | U937-Histiocytic lymphoma | 18.2 |
| lung cancer | | | |
| NCI-H146-Small cell | 30.6 | KU-812-Myelogenous | 6.0 |
| lung cancer | | leukemia | |
| NCI-H526-Small cell | 57.4 | 769-P-Clear cell renal | 11.3 |
| lung cancer | | carcinoma | |
| NCI-N417-Small cell | 15.2 | Caki-2-Clear cell renal | 10.4 |
| lung cancer | | carcinoma | |
| NCI-H82-Small cell | 36.9 | SW 839-Clear cell renal | 2.2 |
| lung cancer | | carcinoma | |
| NCI-H157-Squamous | 51.1 | G401-Wilms' tumor | 6.2 |
| cell lung cancer | | | |
| (metastasis) | | | |
| NCI-H1155-Large cell | 26.6 | Hs766T-Pancreatic | 42.9 |
| lung cancer | | carcinoma (LN metastasis) | |
| NCI-H1299-Large cell | 44.4 | CAPAN-1-Pancreatic | 5.0 |
| lung cancer | | adenocarcinoma (liver | |
| | | metastasis) | |
| NCI-H727-Lung | 47.0 | SU86.86-Pancreatic | 28.1 |
| carcinoid | | carcinoma (liver metastasis) | |
| NCI-UMC-11-Lung | 60.3 | BxPC-3-Pancreatic | 6.3 |
| carcinoid | | adenocarcinoma | |
| LX-1-Small cell lung | 23.5 | HPAC-Pancreatic | 7.4 |
| cancer | | adenocarcinoma | |
| Colo-205-Colon cancer | 29.5 | MIA PaCa-2-Pancreatic | 3.6 |
| | | carcinoma | |
| KM12-Colon cancer | 24.0 | CFPAC-1-Pancreatic ductal | 40.9 |
| | | adenocarcinoma | |
| KM20L2-Colon cancer | 8.4 | PANC-1-Pancreatic | 20.9 |
| | | epithelioid ductal carcinoma | |
| NCI-H716-Colon | 23.3 | T24-Bladder carcinma | 13.1 |
| cancer | | (transitional cell) | |
| SW-48-Colon | 27.0 | 5637-Bladder carcinoma | 11.0 |
| adenocarcinoma | | | |
| SW1116-Colon | 10.0 | HT-1197-Bladder | 9.2 |
| adenocarcinoma | | carcinoma | |
| LS 174T-Colon | 9.9 | UM-UC-3-Bladder | 7.2 |
| adenocarcinoma | | carcinma (transitional cell) | |
| SW-948-Colon | 1.1 | A204-Rhabdomyosarcoma | 7.0 |
| adenocarcinoma | | | |
| SW-480-Colon | 8.8 | HT-1080-Fibrosarcoma | 16.6 |
| adenocarcinoma | | | |
| NCI-SNU-5-Gastric | 7.2 | MG-63-Osteosarcoma | 16.7 |
| carcinoma | | | |
| KATO III-Gastric | 32.8 | SK-LMS-1- | 26.4 |
| carcinoma | | Leiomyosarcoma (vulva) | |
| NCI-SNU-16-Gastric | 13.6 | SJRH30- | 16.8 |
| carcinoma | | Rhabdomyosarcoma (met to | |
| | | bone marrow) | |
| NCI-SNU-1-Gastric | 16.0 | A431-Epidermoid | 9.8 |
| carcinoma | | carcinoma | |
| RF-1-Gastric | 2.1 | WM266-4-Melanoma | 12.0 |
| adenocarcinoma | | | |
| RF-48-Gastric | 4.0 | DU 145-Prostate carcinoma | 0.1 |
| adenocarcinoma | | (brain metastasis) | |
| MKN-45-Gastric | 28.7 | MDA-MB-468-Breast | 14.7 |
| carcinoma | | adenocarcinoma | |
| NCI-N87-Gastric | 13.3 | SCC-4-Squamous cell | 0.9 |
| carcinoma | | carcinoma of tongue | |
| OVCAR-5-Ovarian | 2.6 | SCC-9-Squamous cell | 0.3 |
| carcinoma | | carcinoma of tongue | |
| RL95-2-Uterine | 7.2 | SCC-15-Squamous cell | 0.5 |
| carcinoma | | carcinoma of tongue | |
| HelaS3-Cervical | 13.4 | CAL 27-Squamous cell | 21.2 |
| adenocarcinoma | | carcinoma of tongue | |

TABLE CCE
Panel 4D

| | Rel. Exp. (%) | | Rel. Exp. (%) |
|---|---|---|---|
| | Ag2075, Run | | Ag2075, Run |
| Tissue Name | 152787491 | Tissue Name | 152787491 |
| Secondary Th1 act | 46.3 | HUVEC IL-1beta | 25.0 |
| Secondary Th2 act | 39.2 | HUVEC IFN gamma | 33.0 |
| Secondary Tr1 act | 31.9 | HUVEC TNF alpha + | 13.1 |
| | | IFN gamma | |
| Secondary Th1 rest | 11.7 | HUVEC TNF alpha + | 20.7 |
| | | IL4 | |
| Secondary Th2 rest | 13.9 | HUVEC IL-11 | 20.0 |
| Secondary Tr1 rest | 21.6 | Lung Microvascular EC | 22.7 |
| | | none | |
| Primary Th1 act | 30.1 | Lung Microvascular EC | 15.0 |
| | | TNF alpha + IL-1beta | |
| Primary Th2 act | 39.2 | Microvascular Dermal | 26.8 |
| | | EC none | |
| Primary Tr1 act | 54.7 | Microsvasular Dermal | 16.6 |
| | | EC TNF alpha + IL-1beta | |
| Primary Th1 rest | 84.1 | Bronchial epithelium | 4.9 |
| | | TNF alpha + IL1beta | |
| Primary Th2 rest | 48.6 | Small airway epithelium | 19.9 |
| | | none | |
| Primary Tr1 rest | 39.0 | Small airway epithelium | 72.7 |
| | | TNF alpha + IL-1beta | |
| CD45RA CD4 | 21.8 | Coronery artery SMC rest | 27.9 |
| lymphocyte act | | | |
| CD45RO CD4 | 33.0 | Coronery artery SMC | 19.6 |
| lymphocyte act | | TNF alpha + IL-1beta | |
| CD8 lymphocyte act | 25.5 | Astrocytes rest | 26.4 |
| Secondary CD8 | 21.5 | Astrocytes TNF alpha + | 13.2 |
| lymphocyte rest | | IL-1beta | |
| Secondary CD8 | 29.3 | KU-812 (Basophil) rest | 6.3 |
| lymphocyte act | | | |
| CD4 lymphocyte none | 12.7 | KU-812 (Basophil) | 16.0 |
| | | PMA/ionomycin | |
| 2ry Th1/Th2/Tr1_anti- | 25.5 | CCD1106 | 46.0 |
| CD95 CH11 | | (Keratinocytes) none | |
| LAK cells rest | 26.1 | CCD1106 | 4.3 |
| | | (Keratinocytes) | |
| | | TNF alpha + IL-1beta | |
| LAK cells IL-2 | 30.6 | Liver cirrhosis | 2.7 |
| LAK cells IL-2 + IL-12 | 18.2 | Lupus kidney | 3.0 |
| LAK cells IL-2 + IFN | 31.0 | NCI-H292 none | 76.8 |
| gamma | | | |
| LAK cells IL-2 + IL-18 | 31.2 | NCI-H292 IL-4 | 94.6 |
| LAK cells | 9.5 | NCI-H292 IL-9 | 97.3 |
| PMA/ionomycin | | | |
| NK Cells IL-2 rest | 37.4 | NCI-H292 IL-13 | 59.5 |
| Two Way MLR 3 day | 24.0 | NCI-H292 IFN gamma | 51.8 |
| Two Way MLR 5 day | 23.0 | HPAEC none | 23.3 |
| Two Way MLR 7 day | 19.6 | HPAEC TNF alpha + IL- | 15.8 |
| | | 1beta | |
| PBMC rest | 11.4 | Lung fibroblast none | 18.4 |
| PBMC PWM | 72.2 | Lung fibroblast TNF | 12.0 |
| | | alpha + IL-1beta | |
| PBMC PHA-L | 33.9 | Lung fibroblast IL-4 | 35.8 |
| Ramos (B cell) none | 19.6 | Lung fibroblast IL-9 | 25.5 |
| Ramos (B cell) | 100.0 | Lung fibroblast IL-13 | 18.7 |
| ionomycin | | | |
| B lymphocytes PWM | 81.8 | Lung fibroblast IFN | 38.4 |
| | | gamma | |
| B lymphocytes CD40L | 42.3 | Dermal fibroblast | 48.0 |
| and IL-4 | | CCD1070 rest | |
| EOL-1 dbcAMP | 23.7 | Dermal fibroblast | 83.5 |
| | | CCD1070 TNF alpha | |
| EOL-1 dbcAMP | 13.5 | Dermal fibroblast | 13.6 |
| PMA/ionomycin | | CCD1070 IL-1beta | |
| Dendritic cells none | 20.9 | Dermal fibroblast IFN | 13.1 |
| | | gamma | |
| Dendritic cells LPS | 11.5 | Dermal fibroblast IL-4 | 36.6 |
| Dendritic cells anti- | 23.2 | IBD Colitis 2 | 1.8 |
| CD40 | | | |
| Monocytes rest | 19.2 | IBD Crohn's | 2.4 |
| Monocytes LPS | 6.5 | Colon | 26.8 |
| Macrophages rest | 36.1 | Lung | 21.3 |
| Macrophages LPS | 13.3 | Thymus | 41.5 |
| HUVEC none | 37.6 | Kidney | 24.3 |
| HUVEC starved | 58.6 | | |

Panel 1.3D Summary: Ag2075 Highest expression of the CG56870-01 gene is detected in the cerebral cortex (CT=24.2). Thus expression of this gene can be used in distinguishing this sample from other samples in the panel. Furthermore, significant expression of this gene is observed throughout the CNS, including in amygdala, substantia nigra, thalamus, cerebellum, cerebral cortex, and spinal cord. The CG56870-01 gene encodes an Ndr3 homolog which is a putative member of Ndr family. This family consists of proteins from different gene families: Ndr1/RTP/Drg1/NDRG1, Ndr2, and Ndr3 (PFAM: IPR004142). NDRG1 is a cytoplasmic protein involved in stress responses, hormone responses, cell growth, and differentiation. Mutation of this gene was reported to be causative for hereditary motor and sensory neuropathy-Lom. Recently, NDRG4, another memember of Ndr family, was shown to be expressed in neurons of the brain and spinal cord. Its expression was markedly decreased in the brain of Alzheimer's disease patient (Zhou et al., 2001). Therefore, this gene may play a role in central nervous system disorders such as Alzheimer's disease, Parkinson's disease, epilepsy, multiple sclerosis, schizophrenia and depression.

This gene also has moderate levels of expression in adipose, adrenal, thyroid, liver, heart, thyroid and skeletal muscle. Thus, this gene product may be important in the pathogenesis, diagnosis and/or treatment of metabolic and endocrine disease, including Types 1 and 2 diabetes and obesity.

In addition, there appears to be substantial expression in other samples derived from breast cancer cell lines, lung cancer cell lines, renal cancer cell lines and colon cancer cell lines. Thus, therapeutic modulation of this gene could be of benefit in the treatment of breast, lung, renal or colon cancer.

### REFERENCES

1. Zhou R H, Kokame K, Tsukamoto Y, Yutani C, Kato H, Miyata T. (2001) Characterization of the human NDRG gene family: a newly identified member, NDRG4, is specifically expressed in brain and heart. Genomics 73(1):86-97

Ag2075 The expression of this gene appears to be highest in a sample derived from a normal brain tissue. In addition, there appears to be substantial expression in other samples derived from breast cancer cell lines, lung cancer cell lines, renal cancer cell lines and colon cancer cell lines. Thus, the expression of this gene could be used to distinguish normal brain tissue from other samples in the panel. Moreover, therapeutic modulation of this gene could be of benefit in the treatment of breast, lung, renal or colon cancer.

Panel 2.2 Summary: Ag2075 Highest expression of CG56870-01 is detected in breast cancer sample (CT=29.89). Thus expression of this gene can be used in distinguishing this sample from other samples in the panel. In addition, there appears to be substantial expression in other samples derived from breast cancers, kidney cancers and colon cancers. Therefore, therapeutic modulation of this could be of benefit in the treatment of breast, kidney or colon cancer.

Panel 3D Summary: Ag2075 The expression of this gene appears to be highest in a sample derived from a lung cancer cell line (DMS-79)(CT=26.4). In addition, there appears to be substantial expression in other samples derived from pancreatic cancer cell lines, lung cancer cell lines, brain cancer cell lines and cervical cancer cell lines. Thus, the expression of this gene could be used to distinguish DMS-79 cells from other samples in the panel. Moreover, therapeutic modulation of this gene could be of benefit in the treatment of pancreatic, lung, brain or cervical cancer.

Panel 4D Summary: Ag2075 Expression of the CG56870-01 gene is ubiquitous througout this panel, with highest in samples derived from ionomycin treated Ramos (B cell) cells (CT=26.1). Furthermore, expression of this gene is also detected in PWM treated PBMC cells and PWM treated B lymphocytes. Therefore, therapeutic modulation of the express ion or function of this gene may reduce or eliminate the symptoms in patients with autoimmune and inflammatory diseases in which B cells play a part in the initiation or progression of the disease process, such as systemic lupus erythematosus, Crohn's disease, ulcerative colitis, multiple sclerosis, chronic obstructive pulmonary disease, asthma, emphysema, rheumatoid arthritis, or psoriasis.

CD. CG56870-04: N-myc Downstream-Regulated Gene 3

Expression of gene CG56870-04 was assessed using the primer-probe sets Ag5279 and Ag2075, described in Tables CDA and CDB. Results of the RTQ-PCR runs are shown in Tables CDC, CDD, CDE, CDF, CDG, CDH and CDI.

Table CDA. Probe Name Ag5279

TABLE CDA
Probe Name Ag5279

| | | | Start | SEQ ID |
|---|---|---|---|---|
| Primers | Sequences | Length | Position | NO: |
| Forward | 5′-aggctgtgatggcggact-3′ | 18 | 873 | 650 |
| Probe | TET-5′-ttcagcctgggaagttcaccgaggcc-3′-TAMRA | 26 | 912 | 651 |
| Reverse | 5′-gccgagtcatgctggcagat-3′20 | 973 | 652 | |

Table CDB. Probe Name Ag2075

TABLE CDB
Probe Name Ag2075

| | | | Start | SEQ ID |
|---|---|---|---|---|
| Primers | Sequences | Length | Position | NO: |
| Forward | 5′-catggatgaacttcaggatgtt-3′ | 22 | 70 | 653 |
| Probe | TET-5′-cagctcacagagatcaaaccacttct-3′-TAMRA | 26 | 92 | 654 |
| Reverse | 5′-tgacagtcaaagtcctggaagt-3′ | 22 | 141 | 655 |

TABLE CDC
CNS_neurodegeneration_v1.0

| | | | Rel. Exp. |
|---|---|---|---|
| | Rel. Exp. (%) | | (%) Ag5279, |
| | Ag5279, Run | | Run |
| Tissue Name | 230512909 | Tissue Name | 230512909 |
| AD 1 Hippo | 7.7 | Control (Path) 3 | 1.0 |
| | | Temporal Ctx | |
| AD 2 Hippo | 8.4 | Control (Path) 4 | 13.2 |
| | | Temporal Ctx | |
| AD 3 Hippo | 3.4 | AD 1 Occipital | 7.2 |
| | | Ctx | |
| AD 4 Hippo | 2.8 | AD 2 Occipital | 0.0 |
| | | Ctx (Missing) | |
| AD 5 hippo | 100.0 | AD 3 Occipital | 2.1 |
| | | Ctx | |
| AD 6 Hippo | 32.8 | AD 4 Occipital | 13.1 |
| | | Ctx | |
| Control 2 Hippo | 32.3 | AD 5 Occipital | 15.6 |
| | | Ctx | |
| Control 4 Hippo | 2.7 | AD 6 Occipital | 54.7 |
| | | Ctx | |
| Control (Path) 3 | 1.2 | Control 1 Occipital | 1.1 |
| Hippo | | Ctx | |
| AD 1 Temporal Ctx | 6.4 | Control 2 Occipital | 81.2 |
| | | Ctx | |
| AD 2 Temporal Ctx | 17.1 | Control 3 Occipital | 8.1 |
| | | Ctx | |
| AD 3 Temporal Ctx | 2.3 | Control 4 Occipital | 2.5 |
| | | Ctx | |
| AD 4 Temporal Ctx | 8.7 | Control (Path) 1 | 66.0 |
| | | Occipital Ctx | |
| AD 5 Inf Temporal | 79.0 | Control (Path) 2 | 5.6 |
| Ctx | | Occipital Ctx | |
| AD 5 SupTemporal | 6.4 | Control (Path) 3 | 1.5 |
| Ctx | | Occipital Ctx | |
| AD 6 Inf Temporal | 25.7 | Control (Path) 4 | 7.6 |
| Ctx | | Occipital Ctx | |
| AD 6 Sup Temporal | 24.1 | Control 1 Parietal | 2.4 |
| Ctx | | Ctx | |
| Control 1 Temporal | 2.0 | Control 2 Parietal | 10.7 |
| Ctx | | Ctx | |
| Control 2 Temporal | 41.2 | Control 3 Parietal | 16.6 |
| Ctx | | Ctx | |
| Control 3 Temporal | 4.6 | Control (Path) 1 | 75.3 |
| Ctx | | Parietal Ctx | |
| Control 4 Temporal | 2.7 | Control (Path) 2 | 13.7 |
| Ctx | | Parietal Ctx | |
| Control (Path) 1 | 35.4 | Control (Path) 3 | 1.6 |
| Temporal Ctx | | Parietal Ctx | |
| Control (Path) 2 | 27.7 | Control (Path) 4 | 19.3 |
| Temporal Ctx | | Parietal Ctx | |

TABLE CDD
General_screening_panel_v1.5

| | Rel. Exp. | | Rel. Exp. |
|---|---|---|---|
| | (%) Ag5279, | | (%) Ag5279, |
| | Run | | Run |
| Tissue Name | 230509998 | Tissue Name | 230509998 |
| Adipose | 1.3 | Renal ca. TK-10 | 9.2 |
| Melanoma* | 7.6 | Bladder | 2.4 |
| Hs688(A).T | | | |
| Melanoma* | 8.0 | Gastric ca. (liver met.) | 8.7 |
| Hs688(B).T | | NCI-N87 | |
| Melanoma* M14 | 12.2 | Gastric ca. KATO III | 21.8 |
| Melanoma* | 13.8 | Colon ca. SW-948 | 4.6 |
| LOXIMVI | | | |
| Melanoma* SK- | 8.2 | Colon ca. SW480 | 14.1 |
| MEL-5 | | | |
| Squamous cell | 6.5 | Colon ca.* (SW480 | 10.4 |
| carcinoma SCC-4 | | met) SW620 | |
| Testis Pool | 8.2 | Colon ca. HT29 | 12.3 |
| Prostate ca.* (bone | 2.3 | Colon ca. HCT-116 | 12.2 |
| met) PC-3 | | | |
| Prostate Pool | 6.2 | Colon ca. CaCo-2 | 17.8 |
| Placenta | 3.5 | Colon cancer tissue | 4.9 |
| Uterus Pool | 1.4 | Colon ca. SW1116 | 2.6 |
| Ovarian ca. | 8.8 | Colon ca. Colo-205 | 7.3 |
| OVCAR-3 | | | |
| Ovarian ca. SK- | 20.4 | Colon ca. SW-48 | 5.9 |
| OV-3 | | | |
| Ovarian ca. | 5.6 | Colon Pool | 5.1 |
| OVCAR-4 | | | |
| Ovarian ca. | 10.9 | Small Intestine Pool | 3.9 |
| OVCAR-5 | | | |
| Ovarian ca. | 6.0 | Stomach Pool | 2.3 |
| IGROV-1 | | | |
| Ovarian ca. | 5.5 | Bone Marrow Pool | 1.5 |
| OVCAR-8 | | | |
| Ovary | 5.7 | Fetal Heart | 7.5 |
| Breast ca. MCF-7 | 5.0 | Heart Pool | 3.3 |
| Breast ca. MDA- | 23.0 | Lymph Node Pool | 5.1 |
| MB-231 | | | |
| Breast ca. BT 549 | 21.9 | Fetal Skeletal Muscle | 3.1 |
| Breast ca. T47D | 10.6 | Skeletal Muscle Pool | 4.0 |
| Breast ca. MDA-N | 5.8 | Spleen Pool | 2.2 |
| Breast Pool | 5.9 | Thymus Pool | 2.9 |
| Trachea | 5.0 | CNS cancer | 20.4 |
| | | (glio/astro) U87-MG | |
| Lung | 2.5 | CNS cancer | 16.8 |
| | | (glio/astro) U-118-MG | |
| Fetal Lung | 8.7 | CNS cancer | 10.7 |
| | | (neuro; met) SK-N-AS | |
| Lung ca. NCI-N417 | 3.7 | CNS cancer (astro) | 7.0 |
| | | SF-539 | |
| Lung ca. LX-1 | 12.1 | CNS cancer (astro) | 21.3 |
| | | SNB-75 | |
| Lung ca. NCI-H146 | 5.8 | CNS cancer (glio) | 5.8 |
| | | SNB-19 | |
| Lung ca. SHP-77 | 7.9 | CNS cancer (glio) SF- | 12.2 |
| | | 295 | |
| Lung ca. A549 | 14.1 | Brain (Amygdala) | 19.3 |
| | | Pool | |
| Lung ca. NCI-H526 | 5.1 | Brain (cerebellum) | 100.0 |
| Lung ca. NCI-H23 | 11.2 | Brain (fetal) | 20.0 |
| Lung ca. NCI-H460 | 2.9 | Brain (Hippocampus) | 17.4 |
| | | Pool | |
| Lung ca. HOP-62 | 4.6 | Cerebral Cortex Pool | 23.7 |
| Lung ca. NCI-H522 | 13.6 | Brain (Substantia | 17.9 |
| | | nigra) Pool | |
| Liver | 0.5 | Brain (Thalamus) Pool | 34.2 |
| Fetal Liver | 3.2 | Brain (whole) | 46.3 |
| Liver ca. HepG2 | 5.3 | Spinal Cord Pool | 9.5 |
| Kidney Pool | 7.1 | Adrenal Gland | 11.8 |
| Fetal Kidney | 5.7 | Pituitary gland Pool | 2.9 |
| Renal ca. 786-0 | 6.3 | Salivary Gland | 3.5 |
| Renal ca. A498 | 11.0 | Thyroid (female) | 2.0 |
| Renal ca. ACHN | 6.0 | Pancreatic ca. | 5.6 |
| | | CAPAN2 | |
| Renal ca. UO-31 | 8.0 | Pancreas Pool | 6.2 |

TABLE CDE
Panel 1.3D

| | Rel. Exp. (%) | | Rel. Exp. (%) |
|---|---|---|---|
| | Ag2075, Run | | Ag2075, |
| Tissue Name | Run 152355202 | Tissue Name | Run 152355202 |
| Liver adenocarcinoma | 11.9 | Kidney (fetal) | 1.4 |
| Pancreas | 0.8 | Renal ca. 786-0 | 3.5 |
| Pancreatic ca. CAPAN 2 | 1.6 | Renal ca. A498 | 12.9 |
| Adrenal gland | 2.4 | Renal ca. RXF 393 | 1.5 |
| Thyroid | 1.8 | Renal ca. ACHN | 1.7 |
| Salivary gland | 1.2 | Renal ca. UO-31 | 6.8 |
| Pituitary gland | 2.4 | Renal ca. TK-10 | 2.8 |
| Brain (fetal) | 3.2 | Liver | 0.4 |
| Brain (whole) | 25.2 | Liver (fetal) | 0.8 |
| Brain (amygdala) | 14.1 | Liver ca. | 6.7 |
| | | (hepatoblast) HepG2 | |
| Brain (cerebellum) | 10.8 | Lung | 2.1 |
| Brain (hippocampus) | 39.8 | Lung (fetal) | 3.2 |
| Brain (substantia nigra) | 2.7 | Lung ca. (small cell) | 4.1 |
| | | LX-1 | |
| Brain (thalamus) | 12.4 | Lung ca. (small cell) | 3.8 |
| | | NCI-H69 | |
| Cerebral Cortex | 100.0 | Lung ca. (s.cell var.) | 3.8 |
| | | SHP-77 | |
| Spinal cord | 3.5 | Lung ca. (large | 0.6 |
| | | cell)NCI-H460 | |
| glio/astro U87-MG | 6.4 | Lung ca. (non-sm. | 3.0 |
| | | cell) A549 | |
| glio/astro U-118-MG | 10.4 | Lung ca. (non-s.cell) | 7.7 |
| | | NCI-H23 | |
| astrocytoma SW1783 | 5.1 | Lung ca. (non-s.cell) | 3.1 |
| | | HOP-62 | |
| neuro*; met SK-N-AS | 6.2 | Lung ca. (non-s.cl) | 6.3 |
| | | NCI-H522 | |
| astrocytoma SF-539 | 5.4 | Lung ca. (squam.) | 1.4 |
| | | SW 900 | |
| astrocytoma SNB-75 | 6.7 | Lung ca. (squam.) | 1.4 |
| | | NCI-H596 | |
| glioma SNB-19 | 3.1 | Mammary gland | 2.4 |
| glioma U251 | 2.0 | Breast ca.* (pl.ef) | 1.8 |
| | | MCF-7 | |
| glioma SF-295 | 3.4 | Breast ca.* (pl.ef) | 11.6 |
| | | MDA-MB-231 | |
| Heart (fetal) | 6.7 | Breast ca.* (pl.ef) | 9.0 |
| | | T47D | |
| Heart | 1.4 | Breast ca. BT-549 | 4.8 |
| Skeletal muscle (fetal) | 18.0 | Breast ca. MDA-N | 4.2 |
| Skeletal muscle | 0.7 | Ovary | 13.4 |
| Bone marrow | 0.9 | Ovarian ca. | 1.8 |
| | | OVCAR-3 | |
| Thymus | 0.9 | Ovarian ca. | 1.6 |
| | | OVCAR-4 | |
| Spleen | 3.7 | Ovarian ca. | 1.8 |
| | | OVCAR-5 | |
| Lymph node | 1.9 | Ovarian ca. | 4.9 |
| | | OVCAR-8 | |
| Colorectal | 3.3 | Ovarian ca. IGROV-1 | 1.6 |
| Stomach | 2.7 | Ovarian ca.* | 5.8 |
| | | (ascites) SK-OV-3 | |
| Small intestine | 2.5 | Uterus | 2.4 |
| Colon ca. SW480 | 10.4 | Placenta | 1.2 |
| Colon ca.* | 3.7 | Prostate | 6.0 |
| SW620(SW480 met) | | | |
| Colon ca. HT29 | 4.6 | Prostate ca.* (bone | 4.5 |
| | | met)PC-3 | |
| Colon ca. HCT-116 | 3.6 | Testis | 1.7 |
| Colon ca. CaCo-2 | 10.3 | Melanoma | 3.8 |
| | | Hs688(A).T | |
| Colon ca. | 3.2 | Melanoma* (met) | 4.4 |
| tissue(ODO3866) | | Hs688(B).T | |
| Colon ca. HCC-2998 | 2.6 | Melanoma UACC- | 1.4 |
| | | 62 | |
| Gastric ca.* (liver met) | 3.1 | Melanoma M14 | 1.8 |
| NCI-N87 | | | |
| Bladder | 0.8 | Melanoma LOX | 3.3 |
| | | IMVI | |
| Trachea | 2.0 | Melanoma* (met) | 2.4 |
| | | SK-MEL-5 | |
| Kidney | 1.3 | Adipose | 1.2 |

TABLE CDF
Panel 2.2

| | Rel. Exp. (%) | | Rel. Exp. (%) |
|---|---|---|---|
| | Ag2075, Run | | Ag2075, |
| Tissue Name | 174255357 | Tissue Name | Run 174255357 |
| Normal Colon | 27.7 | Kidney Margin | 46.3 |
| | | (OD04348) | |
| Colon cancer | 52.9 | Kidney malignant | 19.2 |
| (OD06064) | | cancer (OD06204B) | |
| Colon Margin | 30.1 | Kidney normal | 14.3 |
| (OD06064) | | adjacent tissue | |
| | | (OD06204E) | |
| Colon cancer | 5.5 | Kidney Cancer | 66.0 |
| (OD06159) | | (OD04450-01) | |
| Colon Margin | 21.0 | Kidney Margin | 19.8 |
| (OD06159) | | (OD04450-03) | |
| Colon cancer | 22.2 | Kidney Cancer | 3.0 |
| (OD06297-04) | | 8120613 | |
| Colon Margin | 41.8 | Kidney Margin | 11.0 |
| (OD06297-05) | | 8120614 | |
| CC Gr.2 ascend colon | 4.0 | Kidney Cancer | 6.8 |
| (ODO3921) | | 9010320 | |
| CC Margin (ODO3921) | 6.7 | Kidney Margin | 9.7 |
| | | 9010321 | |
| Colon cancer metastasis | 11.1 | Kidney Cancer | 35.6 |
| (OD06104) | | 8120607 | |
| Lung Margin | 42.6 | Kidney Margin | 13.7 |
| (OD06104) | | 8120608 | |
| Colon mets to lung | 17.8 | Normal Uterus | 59.5 |
| (OD04451-01) | | | |
| Lung Margin | 9.3 | Uterine Cancer 064011 | 9.7 |
| (OD04451-02) | | | |
| Normal Prostate | 51.4 | Normal Thyroid | 8.8 |
| Prostate Cancer | 23.0 | Thyroid Cancer | 11.2 |
| (OD04410) | | 064010 | |
| Prostate Margin | 19.3 | Thyroid Cancer | 17.9 |
| (OD04410) | | A302152 | |
| Normal Ovary | 22.8 | Thyroid Margin | 5.5 |
| | | A302153 | |
| Ovarian cancer | 9.9 | Normal Breast | 33.2 |
| (OD06283-03) | | | |
| Ovarian Margin | 17.1 | Breast Cancer | 8.5 |
| (OD06283-07) | | (OD04566) | |
| Ovarian Cancer 064008 | 18.0 | Breast Cancer 1024 | 36.1 |
| Ovarian cancer | 6.6 | Breast Cancer | 18.4 |
| (OD06145) | | (OD04590-01) | |
| Ovarian Margin | 12.5 | Breast Cancer Mets | 31.9 |
| (OD06145) | | (OD04590-03) | |
| Ovarian cancer | 14.7 | Breast Cancer | 45.4 |
| (OD06455-03) | | Metastasis (OD04655- | |
| | | 05) | |
| Ovarian Margin | 21.8 | Breast Cancer 064006 | 11.5 |
| (OD06455-07) | | | |
| Normal Lung | 21.9 | Breast Cancer 9100266 | 20.9 |
| Invasive poor diff. lung | 17.6 | Breast Margin | 35.1 |
| adeno (ODO4945-01) | | 9100265 | |
| Lung Margin | 12.2 | Breast Cancer | 9.7 |
| (ODO4945-03) | | A209073 | |
| Lung Malignant Cancer | 8.7 | Breast Margin | 22.2 |
| (OD03126) | | A2090734 | |
| Lung Margin | 7.4 | Breast cancer | 100.0 |
| (OD03126) | | (OD06083) | |
| Lung Cancer | 9.9 | Breast cancer node | 63.7 |
| (OD05014A) | | metastasis (OD06083) | |
| Lung Margin | 21.8 | Normal Liver | 9.9 |
| (OD05014B) | | | |
| Lung cancer | 5.1 | Liver Cancer 1026 | 5.6 |
| (OD06081) | | | |
| Lung Margin | 7.9 | Liver Cancer 1025 | 13.5 |
| (OD06081) | | | |
| Lung Cancer | 17.4 | Liver Cancer 6004-T | 4.8 |
| (OD04237-01) | | | |
| Lung Margin | 24.0 | Liver Tissue 6004-N | 9.3 |
| (OD04237-02) | | | |
| Ocular Melanoma | 9.7 | Liver Cancer 6005-T | 15.7 |
| Metastasis | | | |
| Ocular Melanoma | 4.6 | Liver Tissue 6005-N | 20.4 |
| Margin (Liver) | | | |
| Melanoma Metastasis | 19.8 | Liver Cancer 064003 | 10.7 |
| Melanoma Margin | 21.6 | Normal Bladder | 8.0 |
| (Lung) | | | |
| Normal Kidney | 11.0 | Bladder Cancer 1023 | 10.5 |
| Kidney Ca, Nuclear | 37.6 | Bladder Cancer | 17.3 |
| grade 2 (OD04338) | | A302173 | |
| Kidney Margin | 22.1 | Normal Stomach | 37.9 |
| (OD04338) | | | |
| Kidney Ca Nuclear | 21.6 | Gastric Cancer | 11.1 |
| grade 1/2 (OD04339) | | 9060397 | |
| Kidney Margin | 12.9 | Stomach Margin | 20.6 |
| (OD04339) | | 9060396 | |
| Kidney Ca, Clear cell | 6.5 | Gastric Cancer | 22.7 |
| type (OD04340) | | 9060395 | |
| Kidney Margin | 18.4 | Stomach Margin | 36.1 |
| (OD04340) | | 9060394 | |
| Kidney Ca, Nuclear | 12.9 | Gastric Cancer 064005 | 8.1 |
| grade 3 (OD04348) | | | |

TABLE CDG
Panel 3D

| | Rel. Exp. (%) | | Rel. Exp. (%) |
|---|---|---|---|
| | Ag2075, Run | | Ag2075, Run |
| Tissue Name | 164750734 | Tissue Name | 164750734 |
| Daoy-Medulloblastoma | 6.7 | Ca Ski-Cervical epidermoid | 50.3 |
| | | carcinoma (metastasis) | |
| TE671- | 9.0 | ES-2-Ovarian clear cell | 13.9 |
| Medulloblastoma | | carcinoma | |
| D283 Med- | 35.4 | Ramos-Stimulated with | 2.7 |
| Medulloblastoma | | PMA/ionomycin 6 h | |
| PFSK-1-Primitive | 15.4 | Ramos-Stimulated with | 3.3 |
| Neuroectodermal | | PMA/ionomycin 14 h | |
| XF-498-CNS | 4.4 | MEG-01-Chronic | 3.4 |
| | | myelogenous leukemia | |
| | | (megokaryoblast) | |
| SNB-78-Glioma | 23.3 | Raji-Burkitt's lymphoma | 3.7 |
| SF-268-Glioblastoma | 13.6 | Daudi-Burkitt's lymphoma | 6.5 |
| T98G-Glioblastoma | 18.6 | U266-B-cell plasmacytoma | 8.4 |
| SK-N-SH- | 17.0 | CA46-Burkitt's lymphoma | 7.2 |
| Neuroblastoma | | | |
| (metastasis) | | | |
| SF-295-Glioblastoma | 8.4 | RL-non-Hodgkin's B-cell | 3.5 |
| | | lymphoma | |
| Cerebellum | 74.7 | JM1-pre-B-cell lymphoma | 3.1 |
| Cerebellum | 62.4 | Jurkat-T cell leukemia | 11.6 |
| NCI-H292- | 45.7 | TF-1-Erythroleukemia | 13.4 |
| Mucoepidermoid lung | | | |
| carcinoma | | | |
| DMS-114-Small cell | 10.9 | HUT 78-T-cell lymphoma | 10.0 |
| lung cancer | | | |
| DMS-79-Small cell | 100.0 | U937-Histiocytic lymphoma | 18.2 |
| lung cancer | | | |
| NCI-H146-Small cell | 30.6 | KU-812-Myelogenous | 6.0 |
| lung cancer | | leukemia | |
| NCI-H526-Small cell | 57.4 | 769-P-Clear cell renal | 11.3 |
| lung cancer | | carcinoma | |
| NCI-N417-Small cell | 15.2 | Caki-2-Clear cell renal | 10.4 |
| lung cancer | | carcinoma | |
| NCI-H82-Small cell | 36.9 | SW 839-Clear cell renal | 2.2 |
| lung cancer | | carcinoma | |
| NCI-H157-Squamous | 51.1 | G401-Wilms' tumor | 6.2 |
| cell lung cancer | | | |
| (metastasis) | | | |
| NCI-H1155-Large cell | 26.6 | Hs766T-Pancreatic | 42.9 |
| lung cancer | | carcinoma (LN metastasis) | |
| NCI-H1299-Large cell | 44.4 | CAPAN-1-Pancreatic | 5.0 |
| lung cancer | | adenocarcinoma (liver | |
| | | metastasis) | |
| NCI-H727-Lung | 47.0 | SU86.86-Pancreatic | 28.1 |
| carcinoid | | carcinoma (liver metastasis) | |
| NCI-UMC-11-Lung | 60.3 | BxPC-3-Pancreatic | 6.3 |
| carcinoid | | adenocarcinoma | |
| LX-1-Small cell lung | 23.5 | HPAC-Pancreatic | 7.4 |
| cancer | | adenocarcinoma | |
| Colo-205-Colon cancer | 29.5 | MIA PaCa-2-Pancreatic | 3.6 |
| | | carcinoma | |
| KM12-Colon cancer | 24.0 | CFPAC-1-Pancreatic ductal | 40.9 |
| | | adenocarcinoma | |
| KM20L2-Colon cancer | 8.4 | PANC-1-Pancreatic | 20.9 |
| | | epithelioid ductal carcinoma | |
| NCI-H716-Colon | 23.3 | T24-Bladder carcinma | 13.1 |
| cancer | | (transitional cell) | |
| SW-48-Colon | 27.0 | 5637-Bladder carcinoma | 11.0 |
| adenocarcinoma | | | |
| SW1116-Colon | 10.0 | HT-1197-Bladder | 9.2 |
| adenocarcinoma | | carcinoma | |
| LS 174T-Colon | 9.9 | UM-UC-3-Bladder | 7.2 |
| adenocarcinoma | | carcinma (transitional cell) | |
| SW-948-Colon | 1.1 | A204-Rhabdomyosarcoma | 7.0 |
| adenocarcinoma | | | |
| SW-480-Colon | 8.8 | HT-1080-Fibrosarcoma | 16.6 |
| adenocarcinoma | | | |
| NCI-SNU-5-Gastric | 7.2 | MG-63-Osteosarcoma | 16.7 |
| carcinoma | | | |
| KATO III-Gastric | 32.8 | SK-LMS-1- | 26.4 |
| carcinoma | | Leiomyosarcoma (vulva) | |
| NCI-SNU-16-Gastric | 13.6 | SJRH30- | 16.8 |
| carcinoma | | Rhabdomyosarcoma (met to | |
| | | bone marrow) | |
| NCI-SNU-1-Gastric | 16.0 | A431-Epidermoid | 9.8 |
| carcinoma | | carcinoma | |
| RF-1-Gastric | 2.1 | WM266-4-Melanoma | 12.0 |
| adenocarcinoma | | | |
| RF-48-Gastric | 4.0 | DU 145-Prostate carcinoma | 0.1 |
| adenocarcinoma | | (brain metastasis) | |
| MKN-45-Gastric | 28.7 | MDA-MB-468-Breast | 14.7 |
| carcinoma | | adenocarcinoma | |
| NCI-N87-Gastric | 13.3 | SCC-4-Squamous cell | 0.9 |
| carcinoma | | carcinoma of tongue | |
| OVCAR-5-Ovarian | 2.6 | SCC-9-Squamous cell | 0.3 |
| carcinoma | | carcinoma of tongue | |
| RL95-2-Uterine | 7.2 | SCC-15-Squamous cell | 0.5 |
| carcinoma | | carcinoma of tongue | |
| HelaS3-Cervical | 13.4 | CAL 27-Squamous cell | 21.2 |
| adenocarcinoma | | carcinoma of tongue | |

TABLE CDH
Panel 4.1D

| | Rel. Exp. (%) | | Rel. Exp. (%) |
|---|---|---|---|
| | Ag5279, Run | | Ag5279, Run |
| Tissue Name | 230472927 | Tissue Name | 230472927 |
| Secondary Th1 act | 59.0 | HUVEC IL-1beta | 41.5 |
| Secondary Th2 act | 91.4 | HUVEC IFN gamma | 71.7 |
| Secondary Tr1 act | 27.5 | HUVEC TNF alpha + | 17.4 |
| | | IFN gamma | |
| Secondary Th1 rest | 7.5 | HUVEC TNF alpha + | 20.7 |
| | | IL4 | |
| Secondary Th2 rest | 8.4 | HUVEC IL-11 | 32.3 |
| Secondary Tr1 rest | 2.6 | Lung Microvascular EC | 57.4 |
| | | none | |
| Primary Th1 act | 26.8 | Lung Microvascular EC | 14.6 |
| | | TNF alpha + IL-1beta | |
| Primary Th2 act | 55.1 | Microvascular Dermal | 10.9 |
| | | EC none | |
| Primary Tr1 act | 55.5 | Microsvasular Dermal | 13.2 |
| | | EC TNF alpha + IL-1beta | |
| Primary Th1 rest | 3.2 | Bronchial epithelium | 17.4 |
| | | TNF alpha + IL1beta | |
| Primary Th2 rest | 13.7 | Small airway epithelium | 18.8 |
| | | none | |
| Primary Tr1 rest | 7.6 | Small airway epithelium | 56.3 |
| | | TNF alpha + IL-1beta | |
| CD45RA CD4 | 31.9 | Coronery artery SMC rest | 31.9 |
| lymphocyte act | | | |
| CD45RO CD4 | 48.3 | Coronery artery SMC | 37.1 |
| lymphocyte act | | TNF alpha + IL-1beta | |
| CD8 lymphocyte act | 21.2 | Astrocytes rest | 28.3 |
| Secondary CD8 | 36.1 | Astrocytes TNF alpha + | 15.8 |
| lymphocyte rest | | IL-1beta | |
| Secondary CD8 | 8.0 | KU-812 (Basophil) rest | 15.2 |
| lymphocyte act | | | |
| CD4 lymphocyte none | 8.4 | KU-812 (Basophil) | 17.0 |
| | | PMA/ionomycin | |
| 2ry Th1/Th2/Tr1_anti- | 8.9 | CCD1106 | 71.2 |
| CD95 CH11 | | (Keratinocytes) none | |
| LAK cells rest | 26.1 | CCD1106 | 42.0 |
| | | (Keratinocytes) | |
| | | TNF alpha + IL-1beta | |
| LAK cells IL-2 | 16.3 | Liver cirrhosis | 15.5 |
| LAK cells IL-2 + IL-12 | 2.0 | NCI-H292 none | 45.4 |
| LAK cells IL-2 + IFN | 12.5 | NCI-H292 IL-4 | 63.7 |
| gamma | | | |
| LAK cells IL-2 + IL-18 | 7.5 | NCI-H292 IL-9 | 70.7 |
| LAK cells | 21.9 | NCI-H292 IL-13 | 99.3 |
| PMA/ionomycin | | | |
| NK Cells IL-2 rest | 44.4 | NCI-H292 IFN gamma | 45.4 |
| Two Way MLR 3 day | 21.9 | HPAEC none | 25.9 |
| Two Way MLR 5 day | 13.5 | HPAEC TNF alpha + IL- | 34.9 |
| | | 1beta | |
| Two Way MLR 7 day | 11.3 | Lung fibroblast none | 37.9 |
| PBMC rest | 11.0 | Lung fibroblast TNF | 21.2 |
| | | alpha + IL-1beta | |
| PBMC PWM | 4.5 | Lung fibroblast IL-4 | 31.6 |
| PBMC PHA-L | 18.4 | Lung fibroblast IL-9 | 36.3 |
| Ramos (B cell) none | 14.0 | Lung fibroblast IL-13 | 9.0 |
| Ramos (B cell) | 42.9 | Lung fibroblast IFN | 58.6 |
| ionomycin | | gamma | |
| B lymphocytes PWM | 18.9 | Dermal fibroblast | 43.2 |
| | | CCD1070 rest | |
| B lymphocytes CD40L | 31.0 | Dermal fibroblast | 100.0 |
| and IL-4 | | CCD1070 TNF alpha | |
| EOL-1 dbcAMP | 37.9 | Dermal fibroblast | 23.5 |
| | | CCD1070 IL-1beta | |
| EOL-1 dbcAMP | 14.7 | Dermal fibroblast IFN | 31.4 |
| PMA/ionomycin | | gamma | |
| Dendritic cells none | 26.2 | Dermal fibroblast IL-4 | 45.4 |
| Dendritic cells LPS | 13.2 | Dermal Fibroblasts rest | 61.6 |
| Dendritic cells anti- | 17.0 | Neutrophils TNFA + LPS | 4.2 |
| CD40 | | | |
| Monocytes rest | 11.6 | Neutrophils rest | 31.4 |
| Monocytes LPS | 8.5 | Colon | 1.0 |
| Macrophages rest | 12.9 | Lung | 1.6 |
| Macrophages LPS | 2.6 | Thymus | 4.5 |
| HUVEC none | 36.1 | Kidney | 32.5 |
| HUVEC starved | 36.9 | | |

TABLE CDI
Panel 4D

| | Rel. Exp. (%) | | Rel. Exp. (%) |
|---|---|---|---|
| | Ag2075, Run | | Ag2075, Run |
| Tissue Name | 152787491 | Tissue Name | 152787491 |
| Secondary Th1 act | 46.3 | HUVEC IL-1beta | 25.0 |
| Secondary Th2 act | 39.2 | HUVEC IFN gamma | 33.0 |
| Secondary Tr1 act | 31.9 | HUVEC TNF alpha + | 13.1 |
| | | IFN gamma | |
| Secondary Th1 rest | 11.7 | HUVEC TNF alpha + | 20.7 |
| | | IL4 | |
| Secondary Th2 rest | 13.9 | HUVEC IL-11 | 20.0 |
| Secondary Tr1 rest | 21.6 | Lung Microvascular EC | 22.7 |
| | | none | |
| Primary Th1 act | 30.1 | Lung Microvascular EC | 15.0 |
| | | TNF alpha + IL-1beta | |
| Primary Th2 act | 39.2 | Microvascular Dermal | 26.8 |
| | | EC none | |
| Primary Tr1 act | 54.7 | Microsvasular Dermal | 16.6 |
| | | EC TNF alpha + IL-1beta | |
| Primary Th1 rest | 84.1 | Bronchial epithelium | 4.9 |
| | | TNF alpha + IL1beta | |
| Primary Th2 rest | 48.6 | Small airway epithelium | 19.9 |
| | | none | |
| Primary Tr1 rest | 39.0 | Small airway epithelium | 72.7 |
| | | TNF alpha + IL-1beta | |
| CD45RA CD4 | 21.8 | Coronery artery SMC rest | 27.9 |
| lymphocyte act | | | |
| CD45RO CD4 | 33.0 | Coronery artery SMC | 19.6 |
| lymphocyte act | | TNF alpha + IL-1beta | |
| CD8 lymphocyte act | 25.5 | Astrocytes rest | 26.4 |
| Secondary CD8 | 21.5 | Astrocytes TNF alpha + | 13.2 |
| lymphocyte rest | | IL-1beta | |
| Secondary CD8 | 29.3 | KU-812 (Basophil) rest | 6.3 |
| lymphocyte act | | | |
| CD4 lymphocyte none | 12.7 | KU-812 (Basophil) | 16.0 |
| | | PMA/ionomycin | |
| 2ry Th1/Th2/Tr1_anti- | 25.5 | CCD1106 | 46.0 |
| CD95 CH11 | | (Keratinocytes) none | |
| LAK cells rest | 26.1 | CCD1106 | 4.3 |
| | | (Keratinocytes) | |
| | | TNF alpha + IL-1beta | |
| LAK cells IL-2 | 30.6 | Liver cirrhosis | 2.7 |
| LAK cells IL-2 + IL-12 | 18.2 | Lupus kidney | 3.0 |
| LAK cells IL-2 + IFN | 31.0 | NCI-H292 none | 76.8 |
| gamma | | | |
| LAK cells IL-2 + IL-18 | 31.2 | NCI-H292 IL-4 | 94.6 |
| LAK cells | 9.5 | NCI-H292 IL-9 | 97.3 |
| PMA/ionomycin | | | |
| NK Cells IL-2 rest | 37.4 | NCI-H292 IL-13 | 59.5 |
| Two Way MLR 3 day | 24.0 | NCI-H292 IFN gamma | 51.8 |
| Two Way MLR 5 day | 23.0 | HPAEC none | 23.3 |
| Two Way MLR 7 day | 19.6 | HPAEC TNF alpha + IL- | 15.8 |
| | | 1beta | |
| PBMC rest | 11.4 | Lung fibroblast none | 18.4 |
| PBMC PWM | 72.2 | Lung fibroblast TNF | 12.0 |
| | | alpha + IL-1beta | |
| PBMC PHA-L | 33.9 | Lung fibroblast IL-4 | 35.8 |
| Ramos (B cell) none | 19.6 | Lung fibroblast IL-9 | 25.5 |
| Ramos (B cell) | 100.0 | Lung fibroblast IL-13 | 18.7 |
| ionomycin | | | |
| B lymphocytes PWM | 81.8 | Lung fibroblast IFN | 38.4 |
| | | gamma | |
| B lymphocytes CD40L | 42.3 | Dermal fibroblast | 48.0 |
| and IL-4 | | CCD1070 rest | |
| EOL-1 dbcAMP | 23.7 | Dermal fibroblast | 83.5 |
| | | CCD1070 TNF alpha | |
| EOL-1 dbcAMP | 13.5 | Dermal fibroblast | 13.6 |
| PMA/ionomycin | | CCD1070 IL-1beta | |
| Dendritic cells none | 20.9 | Dermal fibroblast IFN | 13.1 |
| | | gamma | |
| Dendritic cells LPS | 11.5 | Dermal fibroblast IL-4 | 36.6 |
| Dendritic cells anti- | 23.2 | IBD Colitis 2 | 1.8 |
| CD40 | | | |
| Monocytes rest | 19.2 | IBD Crohn's | 2.4 |
| Monocytes LPS | 6.5 | Colon | 26.8 |
| Macrophages rest | 36.1 | Lung | 21.3 |
| Macrophages LPS | 13.3 | Thymus | 41.5 |
| HUVEC none | 37.6 | Kidney | 24.3 |
| HUVEC starved | 58.6 | | |

CNS_neurodegeneration_v1.0 Summary: Ag5279 This panel confirms the expression of the CG56870-04 gene at low levels in the brain in an independent group of individuals. However, no differential expression of this gene was detected between Alzheimer's diseased postmortem brains and those of non-demented controls in this experiment. Please see Panel 1.5 for a discussion of the potential utility of this gene in treatment of central nervous system disorders.

General_screening_panel_v1.5 Summary: Ag5279 Highest expression of the CG56870-01 is detected in cerebral cortex (CT=25.02). Thus, expression of this gene can be used in distinguishing this sample from other samples in the panel. Furthermore, significant expression of this gene is observed throughout the CNS, including in amygdala, substantia nigra, thalamus, cerebellum, cerebral cortex, and spinal cord. The CG56870-01 gene encodes a Ndr3 protein homolog. The Ndr family is comprised of members from different gene families: Ndr1/RTP/Drg1/NDRG1, Ndr2, and Ndr3 (PFAM: IPR004142). NDRG1 is a cytoplasmic protein involved in stress responses, hormone responses, cell growth, and differentiation. Mutation of this gene was reported to be causative for hereditary motor and sensory neuropathy-Lom. Recently, NDRG4, another memember of Ndr family, was shown to be expressed in neurons of the brain and spinal cord. Its expression was markedly decreased in the brain of Alzheimer's disease patient (Zhou et al., 2001). Therefore, this gene may play a role in central nervous system disorders such as Alzheimer's disease, Parkinson's disease, epilepsy, multiple sclerosis, schizophrenia and depression.

Among metabolic tissues, this gene is moderately expressed in adipose, adrenal, heart, thyroid, liver, pancreas, pituitary, and skeletal muscle. Thus, this gene product may be important for the pathogenesis, diagnosis, and/or treatment of metabolic and endocrine disease, including Types 1 and 2 diabetes and obesity.

In addition, there appears to be substantial expression in other samples derived from brain cancer cell lines, colon cancer cell lines, breast cancer cell lines and ovarian cancer cell lines. Moreover, therapeutic modulation of this gene could be of benefit in the treatment of brain, colon, breast or ovarian cancer.

### REFERENCES

1. Zhou R H, Kokame K, Tsukamoto Y, Yutani C, Kato H, Miyata T. (2001) Characterization of the human NDRG gene family: a newly identified member, NDRG4, is specifically expressed in brain and heart. Genomics 73(1):86-97

Panel 1.3D Summary: Ag2075 Highest expression of the CG56870-01 gene is detected in the cerebral cortex (CT=24.2). This expression is consistent with expression in Panel 1.5. Please see that panel for discussion of utility of this gene in the central nervous system.

This gene also has moderate levels of expression in adipose, adrenal, thyroid, liver, heart, thyroid and skeletal muscle. Thus, this gene product may be important in the pathogenesis, diagnosis and/or treatment of metabolic and endocrine disease, including Types 1 and 2 diabetes and obesity.

In addition, there appears to be substantial expression in other samples derived from breast cancer cell lines, lung cancer cell lines, renal cancer cell lines and colon cancer cell lines. Thus, therapeutic modulation of this gene could be of benefit in the treatment of breast, lung, renal or colon cancer.

Panel 2.2 Summary: Ag2075 Highest expression of CG56870-01 is detected in breast cancer sample (CT=29.89). Thus expression of this gene can be used in distinguishing this sample from other samples in the panel. In addition, there appears to be substantial expression in other samples derived from breast cancers, kidney cancers and colon cancers. Therefore, therapeutic modulation of this gene could be of benefit in the treatment of breast, kidney or colon cancer.

Panel 3D Summary: Ag2075 The expression of this gene appears to be highest in a sample derived from a lung cancer cell line (DMS-79)(CT=26.4). In addition, there appears to be substantial expression in other samples derived from pancreatic cancer cell lines, lung cancer cell lines, brain cancer cell lines and cervical cancer cell lines. Thus, the expression of this gene could be used to distinguish DMS-79 cells from other samples in the panel. Moreover, therapeutic modulation of this gene could be of benefit in the treatment of pancreatic, lung, brain or cervical cancer.

Panel 4.1D Summary: Ag5279 Expression of the CG56870-01 gene is highest in samples derived from TNF alpha treated dermal fibroblast CCD1070 cells (CT=30.6). Expression of this gene is also prominent in activated secondary and primarey Th1, Th2 and Tr1 cells when compared expression in the corresponding resting cell lines. Thus, this gene may be involved in T lymphocyte function. Therefore, therapeutic modulation to the expression or function of this gene may be as anti-inflammatory therapeutics for T cell-mediated autoimmune and inflammatory diseases, such as asthma, athritis, psoriasis, IBD, and lupus.

Panel 4D Summary: Ag2075 Expression of the CG56870-01 gene is ubiquitous throughout this panel, with highest in samples derived from ionomycin treated Ramos (B cell) cells (CT=26.1). Furthermore, expression of this gene is also detected in PWM treated PBMC cells and PWM treated B lymphocytes. Therefore, therapeutic modulation of the expression or function of this gene may reduce or eliminate the symptoms in patients with autoimmune and inflammatory diseases in which B cells play a part in the initiation or progression of the disease process, such as systemic lupus erythematosus, Crohn's disease, ulcerative colitis, multiple sclerosis, chronic obstructive pulmonary disease, asthma, emphysema, rheumatoid arthritis, or psoriasis.

CE. CG!56870-05: N-myc Downstream-Regulated Gene 3

Expression of gene CG56870-05 was assessed using the primer-probe set Ag5265, described in Table CEA. Results of the RTQ-PCR runs are shown in Tables CEB and CEC.

Table CEA. Probe Name Ag5265

TABLE CEA
Probe Name Ag5265

| | | | Start | SEQ ID |
|---|---|---|---|---|
| Primers | Sequences | Length | Position | NO: |
| Forward | 5′-tgttcagctcacagagatcaaa-3′ | 22 | 18 | 656 |
| Probe | TET-5′-caagaaacttccaggactttgactgtca-3′-TAMRA | 28 | 65 | 657 |
| Reverse | 5′-catccattgtggggtactga-3′ | 20 | 96 | 658 |

TABLE CEB
CNS_neurodegeneration_v1.0

| | | | Rel. Exp. |
|---|---|---|---|
| | Rel. Exp. (%) | | (%) Ag5265, |
| | Ag5265, Run | | Run |
| Tissue Name | 230512714 | Tissue Name | 230512714 |
| AD 1 Hippo | 7.2 | Control (Path) 3 | 2.4 |
| | | Temporal Ctx | |
| AD 2 Hippo | 15.2 | Control (Path) 4 | 10.3 |
| | | Temporal Ctx | |
| AD 3 Hippo | 2.0 | AD 1 Occipital Ctx | 11.5 |
| AD 4 Hippo | 3.3 | AD 2 Occipital Ctx | 0.0 |
| | | (Missing) | |
| AD 5 Hippo | 59.9 | AD 3 Occipital Ctx | 7.0 |
| AD 6 Hippo | 22.1 | AD 4 Occipital Ctx | 6.9 |
| Control 2 Hippo | 14.5 | AD 5 Occipital Ctx | 54.7 |
| Control 4 Hippo | 2.6 | AD 6 Occipital Ctx | 12.2 |
| Control (Path) 3 | 3.9 | Control 1 Occipital | 1.2 |
| Hippo | | Ctx | |
| AD 1 Temporal | 12.7 | Control 2 Occipital | 60.3 |
| Ctx | | Ctx | |
| AD 2 Temporal | 16.0 | Control 3 Occipital | 8.2 |
| Ctx | | Ctx | |
| AD 3 Temporal | 5.5 | Control 4 Occipital | 1.7 |
| Ctx | | Ctx | |
| AD 4 Temporal | 13.9 | Control (Path) 1 | 53.6 |
| Ctx | | Occipital Ctx | |
| AD 5 Inf Temporal | 64.6 | Control (Path) 2 | 5.9 |
| Ctx | | Occipital Ctx | |
| AD 5 Sup | 28.9 | Control (Path) 3 | 2.2 |
| Temporal Ctx | | Occipital Ctx | |
| AD 6 Inf Temporal | 27.0 | Control (Path) 4 | 5.5 |
| Ctx | | Occipital Ctx | |
| AD 6 Sup | 100.0 | Control 1 Parietal | 3.0 |
| Temporal Ctx | | Ctx | |
| Control 1 | 2.6 | Control 2 Parietal | 19.6 |
| Temporal Ctx | | Ctx | |
| Control 2 | 38.2 | Control 3 Parietal | 18.8 |
| Temporal Ctx | | Ctx | |
| Control 3 | 9.7 | Control (Path) 1 | 56.6 |
| Temporal Ctx | | Parietal Ctx | |
| Control 3 | 1.5 | Control (Path) 2 | 14.2 |
| Temporal Ctx | | Parietal Ctx | |
| Control (Path) 1 | 31.4 | Control (Path) 3 | 2.6 |
| Temporal Ctx | | Parietal Ctx | |
| Control (Path) 2 | 18.3 | Control (Path) 4 | 25.0 |
| Temporal Ctx | | Parietal Ctx | |

TABLE CEC
General_screening_panel_v1.5

| | Rel. Exp. | | Rel. Exp. |
|---|---|---|---|
| | (%) Ag5265, | | (%) Ag5265, |
| | Run | | Run |
| Tissue Name | 232936652 | Tissue Name | 232936652 |
| Adipose | 1.1 | Renal ca. TK-10 | 14.8 |
| Melanoma* | 7.9 | Bladder | 2.5 |
| Hs688(A).T | | | |
| Melanoma* | 10.1 | Gastric ca. (liver met.) | 7.3 |
| Hs688(B).T | | NCI-N87 | |
| Melanorna* M14 | 12.4 | Gastric ca. KATO III | 17.8 |
| Melanoma* | 13.7 | Colon ca. SW-948 | 1.8 |
| LOXIMVI | | | |
| Melanoma* SK- | 9.9 | Colon ca. SW480 | 16.0 |
| MEL-5 | | | |
| Squamous cell | 8.3 | Colon ca.* (SW480 | 8.2 |
| carcinoma SCC-4 | | met) SW620 | |
| Testis Pool | 2.2 | Colon ca. HT29 | 6.7 |
| Prostate ca.* (bone | 18.0 | Colon ca. HCT-116 | 15.8 |
| met) PC-3 | | | |
| Prostate Pool | 4.4 | Colon ca. CaCo-2 | 25.5 |
| Placenta | 2.1 | Colon cancer tissue | 2.5 |
| Uterus Pool | 1.9 | Colon ca. SW1116 | 2.3 |
| Ovarian ca. | 9.0 | Colon ca. Colo-205 | 4.8 |
| OVCAR-3 | | | |
| Ovarian ca. SK- | 16.0 | Colon ca. SW-48 | 5.1 |
| OV-3 | | | |
| Ovarian ca. | 4.5 | Colon Pool | 2.8 |
| OVCAR-4 | | | |
| Ovarian ca. | 10.4 | Small Intestine Pool | 2.4 |
| OVCAR-5 | | | |
| Ovarian ca. | 12.0 | Stomach Pool | 1.8 |
| IGROV-1 | | | |
| Ovarian ca. | 4.5 | Bone Marrow Pool | 1.2 |
| OVCAR-8 | | | |
| Ovary | 2.8 | Fetal Heart | 2.3 |
| Breast ca. MCF-7 | 4.9 | Heart Pool | 1.5 |
| Breast ca. MDA- | 30.1 | Lymph Node Pool | 3.0 |
| MB-231 | | | |
| Breast ca. BT 549 | 18.9 | Fetal Skeletal Muscle | 0.9 |
| Breast ca. T47D | 4.0 | Skeletal Muscle Pool | 1.8 |
| Breast ca. MDA-N | 8.4 | Spleen Pool | 2.2 |
| Breast Pool | 3.6 | Thymus Pool | 1.9 |
| Trachea | 3.2 | CNS cancer | 18.3 |
| | | (glio/astro) U87-MG | |
| Lung | 1.7 | CNS cancer | 19.5 |
| | | (glio/astro) U-118-MG | |
| Fetal Lung | 4.8 | CNS cancer | 8.4 |
| | | (neuro; met) SK-N-AS | |
| Lung ca. NCI-N417 | 2.0 | CNS cancer (astro) | 8.8 |
| | | SF-539 | |
| Lung ca. LX-1 | 8.5 | CNS cancer (astro) | 29.1 |
| | | SNB-75 | |
| Lung ca. NCI-H146 | 9.2 | CNS cancer (glio) | 10.9 |
| | | SNB-19 | |
| Lung ca. SHP-77 | 8.2 | CNS cancer (glio) SF- | 17.2 |
| | | 295 | |
| Lung ca. A549 | 15.2 | Brain (Amygdala) | 14.0 |
| | | Pool | |
| Lung ca. NCI-H526 | 3.2 | Brain (cerebellum) | 100.0 |
| Lung ca. NCI-H23 | 16.2 | Brain (fetal) | 10.7 |
| Lung ca. NCI-H460 | 2.7 | Brain (Hippocampus) | 11.4 |
| | | Pool | |
| Lung ca. HOP-62 | 5.6 | Cerebral Cortex Pool | 15.5 |
| Lung ca. NCI-H522 | 13.0 | Brain (Substantia | 14.7 |
| | | nigra) Pool | |
| Liver | 0.9 | Brain (Thalamus) Pool | 17.9 |
| Fetal Liver | 2.4 | Brain (whole) | 25.9 |
| Liver ca. HepG2 | 6.2 | Spinal Cord Pool | 9.0 |
| Kidney Pool | 6.9 | Adrenal Gland | 5.0 |
| Fetal Kidney | 3.1 | Pituitary gland Pool | 1.5 |
| Renal ca. 786-0 | 9.0 | Salivary Gland | 2.3 |
| Renal ca. A498 | 7.6 | Thyroid (female) | 2.0 |
| Renal ca. ACHN | 6.0 | Pancreatic ca. | 6.5 |
| | | CAPAN2 | |
| Renal ca. UO-31 | 6.3 | Pancreas Pool | 3.2 |

CNS_neurodegeneration_v1.0 Summary: Ag5265 This panel confirms the expression of the CG56870-04 gene at low levels in the brain in an independent group of individuals. However, no differential expression of this gene was detected between Alzheimer's diseased postmortem brains and those of non-demented controls in this experiment. Please see Panel 1.5 for a discussion of the potential utility of this gene in treatment of central nervous system disorders.

General_screening_panel_v1.5 Summary: Ag5265 Highest expression of the CG56870-05 gene is detected in cerebral cortex (CT=28.86). Thus, expression of this gene can be used in distinguishing this sample from other samples in the panel. Furthermore, significant expression of this gene is observed throughout the CNS, including in amygdala, substantia nigra, thalamus, cerebellum, cerebral cortex, and spinal cord. The CG56870-05 gene encodes a putative Ndr3 protein. This family consists of proteins from different gene families: Ndr1/RTP/Drg1/NDRG1, Ndr2, and Ndr3 (PFAM: IPR004142). NDRG1 is a cytoplasmic protein involved in stress responses, hormone responses, cell growth, and differentiation. Mutation of this gene was reported to be causative for hereditary motor and sensory neuropathy-Lom. Recently, NDRG4, another memember of Ndr family, was shown to be expressed in neurons of the brain and spinal cord. Its expression was markedly decreased in the brain of Alzheimer's disease patient (Zhou et al., 2001). Therefore, this gene may play a role in central nervous system disorders such as Alzheimer's disease, Parkinson's disease, epilepsy, multiple sclerosis, schizophrenia and depression.

Among metabolic tissues, this gene has low levels of expression in heart, skeletal muscle, adrenal, thyroid, pancreas and pituitary. Therefore, this gene product may be important for the pathogenesis, diagnosis, and/or treatment of metabolic and endocrine disease, including Types 1 and 2 diabetes and obesity.

Overall, this gene is expressed in all the samples on this panel, with slightly higher levels of expression in the cancer cell lines compared to expression in the normal tissues samples.

Panel 4.1D Summary: Ag5265 Expression of this gene is low/undetectable (CTs>34.5) across all of the samples on this panel (data not shown).

sCF. CG59764-01: Ferritin Heavy Chain Like Protein

Expression of gene CG59764-01 was assessed using the primer-probe set Ag3578, described in Table CFA. Results of the RTQ-PCR runs are shown in Tables CFB and CFC.

Table CFA. Probe Name Ag3578

TABLE CFA
Probe Name Ag3578

| | | | Start | SEQ ID |
|---|---|---|---|---|
| Primers | Sequences | Length | Position | NO: |
| Forward | 5′-ctgcgacttcctggagaac-3′ | 19 | 430 | 659 |
| Probe | TET-5′-agcaggccaagaccatcaaagagct-3′-TAMRA | 25 | 462 | 660 |
| Reverse | 5′-tgtgcaggttgctcaggta-3′ | 19 | 494 | 661 |

TABLE CFB
CNS_neurodegeneration_v1.0

| | | | Rel. Exp. |
|---|---|---|---|
| | Rel. Exp. (%) | | (%) Ag3578, |
| | Ag3578, Run | | Run |
| Tissue Name | 210642348 | Tissue Name | 210642348 |
| AD 1 Hippo | 14.0 | Control (Path) 3 | 3.2 |
| | | Temporal Ctx | |
| AD 2 Hippo | 8.2 | Control (Path) 4 | 52.9 |
| | | Temporal Ctx | |
| AD 3 Hippo | 6.7 | AD 1 Occipital Ctx | 22.8 |
| AD 4 Hippo | 2.3 | AD 2 Occipital Ctx | 0.0 |
| | | (Missing) | |
| AD 5 Hippo | 83.5 | AD 3 Occipital Ctx | 7.3 |
| AD 6 Hippo | 29.1 | AD 4 Occipital Ctx | 14.9 |
| Control 2 Hippo | 1.6 | AD 5 Occipital Ctx | 11.4 |
| Control 4 Hippo | 3.1 | AD 6 Occipital Ctx | 19.8 |
| Control (Path) 3 | 8.0 | Control 1 Occipital | 9.0 |
| Hippo | | Ctx | |
| AD 1 Temporal | 19.5 | Control 2 Occipital | 29.5 |
| Ctx | | Ctx | |
| AD 2 Temporal | 22.8 | Control 3 Occipital | 24.0 |
| Ctx | | Ctx | |
| AD 3 Temporal | 17.0 | Control 4 Occipital | 7.4 |
| Ctx | | Ctx | |
| AD 4 Temporal | 16.8 | Control (Path) 1 | 38.4 |
| Ctx | | Occipital Ctx | |
| AD 5 Inf Temporal | 50.3 | Control (Path) 2 | 10.3 |
| Ctx | | Occipital Ctx | |
| AD 5 Sup | 40.6 | Control (Path) 3 | 0.0 |
| Temporal Ctx | | Occipital Ctx | |
| AD 6 Inf Temporal | 63.7 | Control (Path) 4 | 46.0 |
| Ctx | | Occipital Ctx | |
| AD 6 Sup | 100.0 | Control 1 Parietal | 9.2 |
| Temporal Ctx | | Ctx | |
| Control 1 | 9.2 | Control 2 Parietal | 44.1 |
| Temporal Ctx | | Ctx | |
| Control 2 | 13.3 | Control 3 Parietal | 21.0 |
| Temporal Ctx | | Ctx | |
| Control 3 | 21.3 | Control (Path) 1 | 20.2 |
| Temporal Ctx | | Parietal Ctx | |
| Control 3 | 14.2 | Control (Path) 2 | 16.7 |
| Temporal Ctx | | Parietal Ctx | |
| Control (Path) 1 | 35.1 | Control (Path) 3 | 0.0 |
| Temporal Ctx | | Parietal Ctx | |
| Control (Path) 2 | 25.3 | Control (Path) 4 | 45.4 |
| Temporal Ctx | | Parietal Ctx | |

TABLE CFC
General_screening_panel_v1.4

| | Rel. Exp. | | Rel. Exp. |
|---|---|---|---|
| | (%) Ag3578, | | (%) Ag3578, |
| | Run | | Run |
| Tissue Name | 217423081 | Tissue Name | 217423081 |
| Adipose | 8.6 | Renal ca. TK-10 | 19.9 |
| Melanoma* | 0.0 | Bladder | 0.0 |
| Hs688(A).T | | | |
| Melanoma* | 0.0 | Gastric ca. (liver met.) | 15.3 |
| Hs688(B).T | | NCI-N87 | |
| Melanoma* M14 | 0.0 | Gastric ca. KATO III | 16.4 |
| Melanoma* | 0.0 | Colon ca. SW-948 | 22.8 |
| LOXIMVI | | | |
| Melanoma* SK- | 0.0 | Colon ca. SW480 | 5.6 |
| MEL-5 | | | |
| Squamous cell | 8.3 | Colon ca.* (SW480 | 42.0 |
| carcinoma SCC-4 | | met) SW620 | |
| Testis Pool | 27.4 | Colon ca. HT29 | 2.6 |
| Prostate ca.* (bone | 11.0 | Colon ca. HCT-116 | 39.2 |
| met) PC-3 | | | |
| Prostate Pool | 0.0 | Colon ca. CaCo-2 | 13.5 |
| Placenta | 15.3 | Colon cancer tissue | 4.8 |
| Uterus Pool | 0.0 | Colon ca. SW1116 | 0.0 |
| Ovarian ca. | 0.0 | Colon ca. Colo-205 | 4.2 |
| OVCAR-3 | | | |
| Ovarian ca. SK- | 14.4 | Colon ca. SW-48 | 9.3 |
| OV-3 | | | |
| Ovarian ca. | 8.8 | Colon Pool | 27.5 |
| OVCAR-4 | | | |
| Ovarian ca. | 16.6 | Small Intestine Pool | 11.2 |
| OVCAR-5 | | | |
| Ovarian ca. | 13.0 | Stomach Pool | 11.3 |
| IGROV-1 | | | |
| Ovarian ca. | 4.3 | Bone Marrow Pool | 8.7 |
| OVCAR-8 | | | |
| Ovary | 0.0 | Fetal Heart | 17.7 |
| Breast ca. MCF-7 | 0.0 | Heart Pool | 9.9 |
| Breast ca. MDA- | 5.7 | Lymph Node Pool | 22.4 |
| MB-231 | | | |
| Breast ca. BT 549 | 47.3 | Fetal Skeletal Muscle | 10.2 |
| Breast ca. T47D | 8.0 | Skeletal Muscle Pool | 100.0 |
| Breast ca. MDA-N | 0.0 | Spleen Pool | 0.0 |
| Breast Pool | 7.6 | Thymus Pool | 3.5 |
| Trachea | 0.0 | CNS cancer | 0.0 |
| | | (glio/astro) U87-MG | |
| Lung | 3.1 | CNS cancer | 8.1 |
| | | (glio/astro) U-118-MG | |
| Fetal Lung | 7.1 | CNS cancer | 22.4 |
| | | (neuro; met) SK-N-AS | |
| Lung ca. NCI-N417 | 0.0 | CNS cancer (astro) | 8.2 |
| | | SF-539 | |
| Lung ca. LX-1 | 49.7 | CNS cancer (astro) | 25.2 |
| | | SNB-75 | |
| Lung ca. NCI-H146 | 0.0 | CNS cancer (glio) | 10.1 |
| | | SNB-19 | |
| Lung ca. SHP-77 | 0.0 | CNS cancer (glio) SF- | 24.5 |
| | | 295 | |
| Lung ca. A549 | 0.0 | Brain (Amygdala) | 7.8 |
| | | Pool | |
| Lung ca. NCI-H526 | 0.0 | Brain (cerebellum) | 20.3 |
| Lung ca. NCI-H23 | 11.9 | Brain (fetal) | 18.7 |
| Lung ca. NCI-H460 | 2.7 | Brain (Hippocampus) | 14.9 |
| | | Pool | |
| Lung ca. HOP-62 | 10.9 | Cerebral Cortex Pool | 26.2 |
| Lung ca. NCI-H522 | 0.0 | Brain (Substantia | 35.4 |
| | | nigra) Pool | |
| Liver | 0.0 | Brain (Thalamus) Pool | 19.1 |
| Fetal Liver | 0.0 | Brain (whole) | 17.3 |
| Liver ca. HepG2 | 3.2 | Spinal Cord Pool | 9.2 |
| Kidney Pool | 21.5 | Adrenal Gland | 3.7 |
| Fetal Kidney | 21.2 | Pituitary gland Pool | 0.0 |
| Renal ca. 786-0 | 11.7 | Salivary Gland | 0.0 |
| Renal ca. A498 | 9.2 | Thyroid (female) | 6.7 |
| Renal ca. ACHN | 11.5 | Pancreatic ca. | 48.3 |
| | | CAPAN2 | |
| Renal ca. UO-31 | 10.3 | Pancreas Pool | 5.5 |

CNS_neurodegeneration_v1.0 Summary: Ag3578 This panel confirms the expression of the CG59764-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3578 Highest expression of the CG59764-01 gene is detected in sample derived from skeletal muscle (CT=31.2). Thus expression of this gene can be used to distinguish skeletal muscle sample from other samples used in this panel. This gene is also expressed at low but significant levels in heart and adipose. Thus, this gene product may be useful in the treatment of metabolic disorders that involve these tissues, including obesity.

Significant expression of this gene is also associated with samples derived from breast cancer, pancreatic cancer, colon cancer and lung cancer cell lines. Therefore, therapeutic modulation of the activity of this gene or its protein product might be beneficial in the treatment of these cancers.

In addition, this gene is expressed at low levels throughout the CNS, including in amygdala, substantia nigra, thalamus, cerebellum, cerebral cortex, and spinal cord. The CG59764-01 gene encodes a homologue of ferritin heavy chain protein (H-feritin). It has been hypothesized that the up-regulation of the H-ferritin mRNA is part of a mechanism protecting the hippocampus, a seizure-prone area, against a possible overactivation during absence seizures (Lakaye et al., 2000). Therefore, therapeutic modulation of the expression or function of this gene may be useful in the treatment of seizure disorders, such as epilepsy. Furthermore, this gene may play a role in central nervous system disorders such as Alzheimer's disease, Parkinson's disease, multiple sclerosis, schizophrenia and depression.

### REFERENCES

1. Lakaye B, de Borman B, Minet A, Arckens L, Vergnes M, Marescaux C, Grisar T. (2000) Increased expression of mRNA encoding ferritin heavy chain in brain structures of a rat model of absence epilepsy. Exp Neurol 162(1):112-20.

Panel 4.1D Summary: Ag3578 Expression of this gene is low/undetectable (CTs>35) across all of the samples on this panel (data not shown).

CG. CG59710-01: P14

Expression of gene CG59710-01 was assessed using the primer-probe set Ag3512, described in Table CGA. Results of the RTQ-PCR runs are shown in Tables CGB and CGC.

Table CGA. Probe Name Ag3512

TABLE CGA
Probe Name Ag3512

| | | | Start | SEQ ID |
|---|---|---|---|---|
| Primers | Sequences | Length | Position | NO: |
| Forward | 5′-ctttgttctccagcacatctg-3′ | 21 | 211 | 662 |
| Probe | TET-5′-ctacatcatggccgagatctgcaatg-3′-TAMRA | 26 | 232 | 663 |
| Reverse | 5′-cctcgcatytttaggatctg-3′ | 20 | 290 | 664 |

TABLE CGB
CNS_neurodegeneration_v1.0

| | | | Rel. Exp. |
|---|---|---|---|
| | Rel. Exp. (%) | | (%) Ag3512, |
| | Ag3512, Run | | Run |
| Tissue Name | 211004862 | Tissue Name | 211004862 |
| AD 1 Hippo | 23.0 | Control (Path) 3 | 7.2 |
| | | Temporal Ctx | |
| AD 2 Hippo | 31.2 | Control (Path) 4 | 37.1 |
| | | Temporal Ctx | |
| AD 3 Hippo | 9.8 | AD 1 Occipital | 20.0 |
| | | Ctx | |
| AD 4 Hippo | 10.0 | AD 2 Occipital | 0.0 |
| | | Ctx (Missing) | |
| AD 5 Hippo | 85.9 | AD 3 Occipital | 9.0 |
| | | Ctx | |
| AD 6 Hippo | 56.6 | AD 4 Occipital | 21.3 |
| | | Ctx | |
| Control 2 Hippo | 41.5 | AD 5 Occipital | 15.2 |
| | | Ctx | |
| Control 4 Hippo | 21.6 | AD 6 Occipital | 49.0 |
| | | Ctx | |
| Control (Path) 3 | 12.5 | Control 1 Occipital | 6.0 |
| Hippo | | Ctx | |
| AD 1 Temporal Ctx | 31.9 | Control 2 Occipital | 77.4 |
| | | Ctx | |
| AD 2 Temporal Ctx | 41.2 | Control 3 Occipital | 22.1 |
| | | Ctx | |
| AD 3 Temporal Ctx | 11.3 | Control 4 Occipital | 9.8 |
| | | Ctx | |
| AD 4 Temporal Ctx | 21.0 | Control (Path) 1 | 77.9 |
| | | Occipital Ctx | |
| AD 5 Inf Temporal | 100.0 | Control (Path) 2 | 12.6 |
| Ctx | | Occipital Ctx | |
| AD 5 SupTemporal | 42.6 | Control (Path) 3 | 5.4 |
| Ctx | | Occipital Ctx | |
| AD 6 Inf Temporal | 35.1 | Control (Path) 4 | 21.6 |
| Ctx | | Occipital Ctx | |
| AD 6 Sup Temporal | 56.6 | Control 1 Parietal | 11.4 |
| Ctx | | Ctx | |
| Control 1 Temporal | 10.1 | Control 2 Parietal | 52.5 |
| Ctx | | Ctx | |
| Control 2 Temporal | 53.2 | Control 3 Parietal | 21.5 |
| Ctx | | Ctx | |
| Control 3 Temporal | 17.2 | Control (Path) 1 | 68.8 |
| Ctx | | Parietal Ctx | |
| Control 4 Temporal | 15.9 | Control (Path) 2 | 29.7 |
| Ctx | | Parietal Ctx | |
| Control (Path) 1 | 55.1 | Control (Path) 3 | 10.2 |
| Temporal Ctx | | Parietal Ctx | |
| Control (Path) 2 | 39.5 | Control (Path) 4 | 48.3 |
| Temporal Ctx | | Parietal Ctx | |

TABLE CGC
General_screening_panel_v1.4

| | Rel. Exp. | | Rel. Exp. |
|---|---|---|---|
| | (%) Ag3512, | | (%) Ag3512, |
| | Run | | Run |
| Tissue Name | 217240776 | Tissue Name | 217240776 |
| Adipose | 3.3 | Renal ca. TK-10 | 21.8 |
| Melanoma* | 9.7 | Bladder | 5.5 |
| Hs688(A).T | | | |
| Melanoma* | 10.5 | Gastric ca. (liver met.) | 37.1 |
| Hs688(B).T | | NCI-N87 | |
| Melanoma* M14 | 26.4 | Gastric ca. KATO III | 48.6 |
| Melanoma* | 35.4 | Colon ca. SW-948 | 7.2 |
| LOXIMVI | | | |
| Melanoma* SK- | 14.4 | Colon ca. SW480 | 62.4 |
| MEL-5 | | | |
| Squamous cell | 14.9 | Colon ca.* (SW480 | 29.7 |
| carcinoma SCC-4 | | met) SW620 | |
| Testis Pool | 6.7 | Colon ca. HT29 | 14.6 |
| Prostate ca.* (bone | 17.2 | Colon ca. HCT-116 | 24.1 |
| met) PC-3 | | | |
| Prostate Pool | 4.8 | Colon ca. CaCo-2 | 46.0 |
| Placenta | 4.4 | Colon cancer tissue | 13.9 |
| Uterus Pool | 3.1 | Colon ca. SW1116 | 2.1 |
| Ovarian ca. | 20.0 | Colon ca. Colo-205 | 10.8 |
| OVCAR-3 | | | |
| Ovarian ca. SK- | 31.6 | Colon ca. SW-48 | 11.9 |
| OV-3 | | | |
| Ovarian ca. | 19.6 | Colon Pool | 13.4 |
| OVCAR-4 | | | |
| Ovarian ca. | 30.8 | Small Intestine Pool | 7.0 |
| OVCAR-5 | | | |
| Ovarian ca. | 10.2 | Stomach Pool | 4.0 |
| IGROV-1 | | | |
| Ovarian ca. | 14.0 | Bone Marrow Pool | 2.4 |
| OVCAR-8 | | | |
| Ovary | 3.6 | Fetal Heart | 3.3 |
| Breast ca. MCF-7 | 18.2 | Heart Pool | 4.1 |
| Breast ca. MDA- | 33.7 | Lymph Node Pool | 7.5 |
| MB-231 | | | |
| Breast ca. BT 549 | 24.8 | Fetal Skeletal Muscle | 1.8 |
| Breast ca. T47D | 100.0 | Skeletal Muscle Pool | 6.3 |
| Breast ca. MDA-N | 24.8 | Spleen Pool | 10.8 |
| Breast Pool | 7.3 | Thymus Pool | 5.9 |
| Trachea | 2.8 | CNS cancer | 33.7 |
| | | (glio/astro) U87-MG | |
| Lung | 3.1 | CNS cancer | 30.1 |
| | | (glio/astro) U-118-MG | |
| Fetal Lung | 7.0 | CNS cancer | 13.3 |
| | | (neuro; met) SK-N-AS | |
| Lung ca. NCI-N417 | 7.3 | CNS cancer (astro) | 16.2 |
| | | SF-539 | |
| Lung ca. LX-1 | 17.6 | CNS cancer (astro) | 46.3 |
| | | SNB-75 | |
| Lung ca. NCI-H146 | 13.1 | CNS cancer (glio) | 10.6 |
| | | SNB-19 | |
| Lung ca. SHP-77 | 21.0 | CNS cancer (glio) SF- | 27.5 |
| | | 295 | |
| Lung ca. A549 | 25.7 | Brain (Amygdala) | 4.1 |
| | | Pool | |
| Lung ca. NCI-H526 | 15.0 | Brain (cerebellum) | 6.7 |
| Lung ca. NCI-H23 | 30.8 | Brain (fetal) | 6.7 |
| Lung ca. NCI-H460 | 9.3 | Brain (Hippocampus) | 4.4 |
| | | Pool | |
| Lung ca. HOP-62 | 6.7 | Cerebral Cortex Pool | 6.3 |
| Lung ca. NCI-H522 | 15.2 | Brain (Substantia | 6.1 |
| | | nigra) Pool | |
| Liver | 1.3 | Brain (Thalamus) Pool | 5.6 |
| Fetal Liver | 7.7 | Brain (whole) | 4.7 |
| Liver ca. HepG2 | 11.2 | Spinal Cord Pool | 4.5 |
| Kidney Pool | 12.9 | Adrenal Gland | 3.3 |
| Fetal Kidney | 4.2 | Pituitary gland Pool | 2.6 |
| Renal ca. 786-0 | 11.8 | Salivary Gland | 2.5 |
| Renal ca. A498 | 6.1 | Thyroid (female) | 3.5 |
| Renal ca. ACHN | 10.5 | Pancreatic ca. | 15.3 |
| | | CAPAN2 | |
| Renal ca. UO-31 | 18.2 | Pancreas Pool | 7.6 |

CNS_neurodegeneration_v1.0 Summary: Ag3512 This panel confirms the expression of the CG59710-01 gene at low levels in the brain in an independent group of individuals. However, no differential expression of this gene was detected between Alzheimer's diseased postmortem brains and those of non-demented controls in this experiment. However, as seen in panel 1.4, this gene is expressed at low levels throughout the CNS, including in amygdala, substantia nigra, thalamus, cerebellum, cerebral cortex, and spinal cord. Therefore, this gene may play a role in other central nervous system disorders such as Parkinson's disease, epilepsy, multiple sclerosis, schizophrenia and depression.

General_screening_panel_v1.4 Summary: Ag3512 Highest expression of the CG59710-01 gene is detected in a sample derived from a breast cancer cell line (CT=25.3). Therefore, expression of this gene could be used in distinguishing this sample from other samples in the panel. Overall, expression of this gene appears to be associated with the cancer cell lines suggesting a role for this gene product in cellular growth and proliferation. Specifically, significant expression of this gene is associated with CNS cancer, colon cancer, gastric cancer, renal cancer, lung cancer, breast cancer, ovarian cancer, and melanoma cancer cell lines. Therefore, therapeutic modulation of the activity of this gene or its protein product might be beneficial in the treatment of these cancers.

Panel 4.1D Summary: Ag3512 Results from one experiment with the CG59710-01 gene are not included. The amp plot indicates that there were experimental difficulties with this run.

CH. CG59754-02 and CG59754-01: Down Syndrome Cell Adhesion Molecule

Expression of gene CG59754-02 and variant CG59754-01 was assessed using the primer-probe set Ag1305, described in Table CHA.

Table CHA. Probe Name Ag1305

TABLE CHA
Probe Name Ag1305

| | | | Start | SEQ ID |
|---|---|---|---|---|
| Primers | Sequences | Length | Position | NO: |
| Forward | 5-′-gtgagcattgtgtctccagaa-3′ | 21 | 291 | 665 |
| Probe | TET-5′-tttattacctaccacggcgggctgta-3′-TAMRA | 26 | 321 | 666 |
| Reverse | 5′-tcctccttctgtacgtcagaga-3′ | 22 | 349 | 667 |

Panel 4D Summary: Ag1305 Expression of this gene is low/undetectable (CTs>35) across all of the samples on this panel (data not shown).

CI. CG59800-01: Heparan Sulfate D-Glucosaminyl 3-O-Sulfotransferase-3B

Expression of gene CG59800-01 was assessed using the primer-probe set Ag3589, described in Table CIA.

Table CIA. Probe Name Ag3589

TABLE CIA
Probe Name Ag3589

| | | | Start | SEQ ID |
|---|---|---|---|---|
| Primers | Sequences | Length | Position | NO: |
| Forward | 5′-tacatacctgccctgtccatac-3′ | 22 | 88 | 668 |
| Probe | TET-5′-ctacatacctgccccgtccatacctg-3′-TAMRA | 26 | 117 | 669 |
| Reverse | 5′-gtatggacggggcaggtat-3′ | 19 | 121 | 670 |

Results from Panels CNS_neurodegeneration_v1.0, 1.4, 2.2, and 4.1D are not included. The amp plots corresponding to these runs suggest that there were experimental difficulties with these runs.

CJ. CG59761-01: AXIN 1 (Axis Inhibition Protein 1) (Haxin)—Isoform1, Submitted to Study DDSMT on Mar. 21, 2001 by Cmiller; Clone Status=FIS; Novelty=Novel; ORF Start=97, ORF Stop=2833, Frame=1; 2949 bp.

Expression of gene CG59761-01 was assessed using the primer-probe set Ag3577, described in Table CJA. Results of the RTQ-PCR runs are shown in Tables CJB, CJC and CJD.

Table CJA. Probe Name Ag3577

TABLE CJA
Probe Name Ag3577

| | | | Start | SEQ ID |
|---|---|---|---|---|
| Primers | Sequences | Length | Position | NO: |
| Forward | 5′-atacttgaagtgggctgagtca-3′ | 22 | 486 | 671 |
| Probe | TET-5′-cattccctgctggatgaccaagatg-3′-TAMRA | 25 | 511 | 672 |
| Reverse | 5′-aggaaagtcctgaacaggctta-3′ | 22 | 539 | 673 |

TABLE CJB
CNS_neurodegeneration_v1.0

| | | | Rel. Exp. |
|---|---|---|---|
| | Rel. Exp. (%) | | (%) Ag3577, |
| | Ag3577, Run | | Run |
| Tissue Name | 210642177 | Tissue Name | 210642177 |
| AD 1 Hippo | 26.1 | Control (Path) 3 | 7.5 |
| | | Temporal Ctx | |
| AD 2 Hippo | 20.6 | Control (Path) 4 | 26.8 |
| | | Temporal Ctx | |
| AD 3 Hippo | 10.8 | AD 1 Occipital | 23.5 |
| | | Ctx | |
| AD 4 Hippo | 9.1 | AD 2 Occipital | 0.0 |
| | | Ctx (Missing) | |
| AD 5 hippo | 86.5 | AD 3 Occipital | 12.3 |
| | | Ctx | |
| AD 6 Hippo | 48.3 | AD 4 Occipital | 18.9 |
| | | Ctx | |
| Control 2 Hippo | 21.8 | AD 5 Occipital | 24.3 |
| | | Ctx | |
| Control 4 Hippo | 16.6 | AD 6 Occipital | 34.4 |
| | | Ctx | |
| Control (Path) 3 | 4.7 | Control 1 Occipital | 6.2 |
| Hippo | | Ctx | |
| AD 1 Temporal Ctx | 25.7 | Control 2 Occipital | 57.4 |
| | | Ctx | |
| AD 2 Temporal Ctx | 28.3 | Control 3 Occipital | 13.4 |
| | | Ctx | |
| AD 3 Temporal Ctx | 14.5 | Control 4 Occipital | 8.7 |
| | | Ctx | |
| AD 4 Temporal Ctx | 19.8 | Control (Path) 1 | 62.4 |
| | | Occipital Ctx | |
| AD 5 Inf Temporal | 100.0 | Control (Path) 2 | 10.5 |
| Ctx | | Occipital Ctx | |
| AD 5 SupTemporal | 44.1 | Control (Path) 3 | 5.3 |
| Ctx | | Occipital Ctx | |
| AD 6 Inf Temporal | 48.3 | Control (Path) 4 | 20.4 |
| Ctx | | Occipital Ctx | |
| AD 6 Sup Temporal | 47.0 | Control 1 Parietal | 15.9 |
| Ctx | | Ctx | |
| Control 1 Temporal | 11.6 | Control 2 Parietal | 54.3 |
| Ctx | | Ctx | |
| Control 2 Temporal | 35.1 | Control 3 Parietal | 15.5 |
| Ctx | | Ctx | |
| Control 3 Temporal | 14.6 | Control (Path) 1 | 43.5 |
| Ctx | | Parietal Ctx | |
| Control 4 Temporal | 12.9 | Control (Path) 2 | 21.3 |
| Ctx | | Parietal Ctx | |
| Control (Path) 1 | 47.0 | Control (Path) 3 | 7.0 |
| Temporal Ctx | | Parietal Ctx | |
| Control (Path) 2 | 28.5 | Control (Path) 4 | 39.0 |
| Temporal Ctx | | Parietal Ctx | |

TABLE CJC
General_screening_panel_v1.4

| | Rel. Exp. | | Rel. Exp. |
|---|---|---|---|
| | (%) Ag3577, | | (%) Ag3577, |
| | Run | | Run |
| Tissue Name | 217343282 | Tissue Name | 217343282 |
| Adipose | 2.8 | Renal ca. TK-10 | 35.4 |
| Melanoma* | 13.9 | Bladder | 13.3 |
| Hs688(A).T | | | |
| Melanoma* | 13.7 | Gastric ca. (liver met.) | 70.7 |
| Hs688(B).T | | NCI-N87 | |
| Melanoma* M14 | 28.5 | Gastric ca. KATO III | 100.0 |
| Melanoma* | 21.2 | Colon ca. SW-948 | 15.0 |
| LOXIMVI | | | |
| Melanoma* SK- | 24.3 | Colon ca. SW480 | 43.5 |
| MEL-5 | | | |
| Squamous cell | 23.2 | Colon ca.* (SW480 | 47.6 |
| carcinoma SCC-4 | | met) SW620 | |
| Testis Pool | 6.4 | Colon ca. HT29 | 26.2 |
| Prostate ca.* (bone | 32.3 | Colon ca. HCT-116 | 27.2 |
| met) PC-3 | | | |
| Prostate Pool | 5.1 | Colon ca. CaCo-2 | 35.4 |
| Placenta | 6.2 | Colon cancer tissue | 13.1 |
| Uterus Pool | 2.8 | Colon ca. SW1116 | 14.2 |
| Ovarian ca. | 12.5 | Colon ca. Colo-205 | 10.1 |
| OVCAR-3 | | | |
| Ovarian ca. SK- | 56.6 | Colon ca. SW-48 | 21.8 |
| OV-3 | | | |
| Ovarian ca. | 11.4 | Colon Pool | 10.7 |
| OVCAR-4 | | | |
| Ovarian ca. | 42.0 | Small Intestine Pool | 10.4 |
| OVCAR-5 | | | |
| Ovarian ca. | 12.9 | Stomach Pool | 5.6 |
| IGROV-1 | | | |
| Ovarian ca. | 13.1 | Bone Marrow Pool | 5.2 |
| OVCAR-8 | | | |
| Ovary | 7.3 | Fetal Heart | 3.5 |
| Breast ca. MCF-7 | 29.3 | Heart Pool | 4.0 |
| Breast ca. MDA- | 32.3 | Lymph Node Pool | 12.7 |
| MB-231 | | | |
| Breast ca. BT 549 | 30.1 | Fetal Skeletal Muscle | 3.6 |
| Breast ca. T47D | 75.3 | Skeletal Muscle Pool | 10.6 |
| Breast ca. MDA-N | 21.8 | Spleen Pool | 7.3 |
| Breast Pool | 12.3 | Thymus Pool | 13.9 |
| Trachea | 10.8 | CNS cancer | 10.7 |
| | | (glio/astro) U87-MG | |
| Lung | 1.8 | CNS cancer | 42.9 |
| | | (glio/astro) U-118-MG | |
| Fetal Lung | 16.0 | CNS cancer | 25.3 |
| | | (neuro; met) SK-N-AS | |
| Lung ca. NCI-N417 | 7.0 | CNS cancer (astro) | 6.0 |
| | | SF-539 | |
| Lung ca. LX-1 | 79.0 | CNS cancer (astro) | 34.2 |
| | | SNB-75 | |
| Lung ca. NCI-H146 | 12.3 | CNS cancer (glio) | 13.8 |
| | | SNB-19 | |
| Lung ca. SHP-77 | 29.1 | CNS cancer (glio) SF- | 28.1 |
| | | 295 | |
| Lung ca. A549 | 29.1 | Brain (Amygdala) | 5.3 |
| | | Pool | |
| Lung ca. NCI-H526 | 7.4 | Brain (cerebellum) | 34.9 |
| Lung ca. NCI-H23 | 28.3 | Brain (fetal) | 21.5 |
| Lung ca. NCI-H460 | 23.3 | Brain (Hippocampus) | 5.4 |
| | | Pool | |
| Lung ca. HOP-62 | 7.8 | Cerebral Cortex Pool | 6.3 |
| Lung ca. NCI-H522 | 28.5 | Brain (Substantia | 6.6 |
| | | nigra) Pool | |
| Liver | 0.9 | Brain (Thalamus) Pool | 8.1 |
| Fetal Liver | 12.5 | Brain (whole) | 11.6 |
| Liver ca. HepG2 | 26.8 | Spinal Cord Pool | 5.1 |
| Kidney Pool | 14.1 | Adrenal Gland | 10.1 |
| Fetal Kidney | 7.2 | Pituitary gland Pool | 0.0 |
| Renal ca. 786-0 | 13.6 | Salivary Gland | 5.9 |
| Renal ca. A498 | 7.9 | Thyroid (female) | 5.3 |
| Renal ca. ACHN | 22.1 | Pancreatic ca. | 30.8 |
| | | CAPAN2 | |
| Renal ca. UO-31 | 17.7 | Pancreas Pool | 13.9 |

TABLE CJD
Panel 4.1D

| | Rel. Exp. (%) | | Rel. Exp. (%) |
|---|---|---|---|
| | Ag3577, Run | | Ag3577, Run |
| Tissue Name | 169851850 | Tissue Name | 169851850 |
| Secondary Th1 act | 50.0 | HUVEC IL-1beta | 37.9 |
| Secondary Th2 act | 57.0 | HUVEC IFN gamma | 25.5 |
| Secondary Tr1 act | 71.2 | HUVEC TNF alpha + | 35.8 |
| | | IFN gamma | |
| Secondary Th1 rest | 29.5 | HUVEC TNF alpha + | 36.9 |
| | | IL4 | |
| Secondary Th2 rest | 58.2 | HUVEC IL-11 | 14.6 |
| Secondary Tr1 rest | 55.1 | Lung Microvascular EC | 48.0 |
| | | none | |
| Primary Th1 act | 62.9 | Lung Microvascular EC | 45.7 |
| | | TNF alpha + IL-1beta | |
| Primary Th2 act | 61.6 | Microvascular Dermal | 27.5 |
| | | EC none | |
| Primary Tr1 act | 60.7 | Microvascular Dermal | 24.1 |
| | | EC TNF alpha + IL-1beta | |
| Primary Th1 rest | 50.3 | Bronchial epithelium | 39.2 |
| | | TNF alpha + IL1beta | |
| Primary Th2 rest | 61.1 | Small airway epithelium | 20.4 |
| | | none | |
| Primary Tr1 rest | 85.3 | Small airway epithelium | 57.8 |
| | | TNF alpha + IL-1beta | |
| CD45RA CD4 | 47.6 | Coronery artery SMC rest | 13.9 |
| lymphocyte act | | | |
| CD45RO CD4 | 58.6 | Coronery artery SMC | 12.9 |
| lymphocyte act | | TNF alpha + IL-1beta | |
| CD8 lymphocyte act | 55.5 | Astrocytes rest | 16.8 |
| Secondary CD8 | 51.4 | Astrocytes TNF alpha + | 18.7 |
| lymphocyte rest | | IL-1beta | |
| Secondary CD8 | 31.0 | KU-812 (Basophil) rest | 37.1 |
| lymphocyte act | | | |
| CD4 lymphocyte none | 33.2 | KU-812 (Basophil) | 64.2 |
| | | PMA/ionomycin | |
| 2ry Th1/Th2/Tr1_anti- | 54.3 | CCD1106 | 48.0 |
| CD95 CH11 | | (Keratinocytes) none | |
| LAK cells rest | 52.1 | CCD1106 | 43.8 |
| | | (Keratinocytes) | |
| | | TNF alpha + IL-1beta | |
| LAK cells IL-2 | 50.3 | Liver cirrhosis | 7.5 |
| LAK cells IL-2 + IL-12 | 47.0 | NCI-H292 none | 30.6 |
| LAK cells IL-2 + IFN | 62.9 | NCI-H292 IL-4 | 60.3 |
| gamma | | | |
| LAK cells IL-2 + IL-18 | 61.1 | NCI-H292 IL-9 | 72.2 |
| LAK cells | 95.9 | NCI-H292 IL-13 | 57.8 |
| PMA/ionomycin | | | |
| NK Cells IL-2 rest | 100.0 | NCI-H292 IFN gamma | 85.9 |
| Two Way MLR 3 day | 74.7 | HPAEC none | 23.0 |
| Two Way MLR 5 day | 50.7 | HPAEC TNF alpha + IL- | 33.4 |
| | | 1beta | |
| Two Way MLR 7 day | 27.9 | Lung fibroblast none | 15.3 |
| PBMC rest | 58.2 | Lung fibroblast TNF | 17.4 |
| | | alpha + IL-1beta | |
| PBMC PWM | 46.7 | Lung fibroblast IL-4 | 23.7 |
| PBMC PHA-L | 29.9 | Lung fibroblast IL-9 | 29.3 |
| Ramos (B cell) none | 39.2 | Lung fibroblast IL-13 | 30.4 |
| Ramos (B cell) | 42.6 | Lung fibroblast IFN | 36.3 |
| ionomycin | | gamma | |
| B lymphocytes PWM | 31.9 | Dermal fibroblast | 47.3 |
| | | CCD1070 rest | |
| B lymphocytes CD40L | 49.7 | Dermal fibroblast | 94.6 |
| and IL-4 | | CCD1070 TNF alpha | |
| EOL-1 dbcAMP | 59.5 | Dermal fibroblast | 20.6 |
| | | CCD1070 IL-1beta | |
| EOL-1 dbcAMP | 55.1 | Dermal fibroblast IFN | 25.0 |
| PMA/ionomycin | | gamma | |
| Dendritic cells none | 47.6 | Dermal fibroblast IL-4 | 34.6 |
| Dendritic cells LPS | 41.2 | Dermal Fibroblasts rest | 22.4 |
| Dendritic cells anti- | 62.4 | Neutrophils TNFa + LPS | 10.2 |
| CD40 | | | |
| Monocytes rest | 50.3 | Neutrophils rest | 28.9 |
| Monocytes LPS | 63.7 | Colon | 20.2 |
| Macrophages rest | 46.0 | Lung | 21.9 |
| Macrophages LPS | 27.7 | Thymus | 57.0 |
| HUVEC none | 15.8 | Kidney | 15.8 |
| HUVEC starved | 29.9 | | |

CNS_neurodegeneration_v1.0 Summary: Ag3577 This panel confirms the expression of the CG59671-01 gene at low levels in the brain in an independent group of individuals. However, no differential expression of this gene was detected between Alzheimer's diseased postmortem brains and those of non-demented controls in this experiment. As seen in panel 1.4, this gene is expressed at low levels throughout the CNS, including in amygdala, substantia nigra, thalamus, cerebellum, cerebral cortex, and spinal cord. Therefore, this gene may play a role in other central nervous system disorders such as Parkinson's disease, epilepsy, multiple sclerosis, schizophrenia and depression.

General_screening_panel_v1.4 Summary: Ag3577 Highest expression of the CG59671-01 gene is detected in a gastric cancer cell line sample (CTs=27.3). In addition, significant expression of this gene is associated with clusters of cell lines derived from ovarian cancer, breast cancer, and gastric cancer. Therefore, expression of this gene might be used to differentiate between these samples and other samples on this panel and as a marker for these cancers. The CG59671-01 gene encodes an Axin 1 protein, which is known play an important role in Wnt signalling transduction pathway. The Wnt/Wingless signaling transduction pathway plays an important role in both embryonic development and tumorigenesis. Beta-Catenin, a key component of the Wnt signaling pathway, interacts with the TCF/LEF family of transcription factors and activates transcription of Wnt target genes. A number of proteins such as the tumor suppressor APC and Axin are also involved in the regulation of the Wnt signaling pathway. Furthermore, mutations in APC or beta-catenin have been found to be responsible for the genesis of human cancers (Akiyama T, 2000). Recently, Dahmen et al. (2001) have shown presence of a single somatic point mutation in exon 1 (Pro255Ser) and deletion of seven large of AXIN1 (12%) in 86 medulloblastoma (MB) samples and 11 MB cell lines. Therefore, AXIN1 may play a role as tumor suppressor gene in MBs. Furthermore, therapeutic modulation of the activity of this gene or its protein product might be beneficial in the treatment of these cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed in all regions of the CNS examined. Please see Panel CNS_neurodegeneration_v1.0 for discussion of utility of this gene in the central nervous system.

### REFERENCES

1. Akiyama T. (2000) Wnt/beta-catenin signaling. Cytokine Growth Factor Rev 11(4):273-82.

2. Dahmen R P, Koch A, Denkhaus D, Tonn J C, Sorensen N, Berthold F, Behrens J, Birchmeier W, Wiestler O D, Pietsch T. (2001) Deletions of AXIN1, a component of the WNT/wingless pathway, in sporadic medulloblastomas. Cancer Res Oct. 1, 2001;61(19):7039-43

Panel 4.1D Summary: Ag3577 Highest expression of the CG59671-01 gene is detected in resting NK Cells IL-2 cells (CTs=28.3). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CK. CG59708-01 and CG59708-02 and CG59708-03: Ubiquitin Carboxyl-Terminal Hydrolase 21

Expression of gene CG59708-01, full length clone CG59708-03 and variant CG59708-02 was assessed using the primer-probe set Ag3511, described in Table CKA. Results of the RTQ-PCR runs are shown in Tables CKB, CKC and CKD. Please note that CG59708-03 represents a full-length physical clone of the CG59708-01 gene, validating the prediction of the gene sequence.

Table CKA. Probe Name Ag3511

TABLE CKA
Probe Name Ag3511

| | | | Start | SEQ ID |
|---|---|---|---|---|
| Primers | Sequences | Length | Position | NO: |
| Forward | 5′-acccaaaagggtagtagaacga-3′ | 22 | 2431 | 674 |
| Probe | TET-5′-cccttctggaacagtttgcagataaa-3′-TAMRA | 26 | 2454 | 675 |
| Reverse | 5′-gccaccttcataatgctgatt-3′ | 21 | 2503 | 676 |

TABLE CKB
CNS_neurodegeneration_v1.0

| | | | Rel. Exp. |
|---|---|---|---|
| | Rel. Exp. (%) | | (%) Ag3511, |
| | Ag3511, Run | | Run |
| Tissue Name | 210499621 | Tissue Name | 210499621 |
| AD 1 Hippo | 8.2 | Control (Path) 3 | 4.1 |
| | | Temporal Ctx | |
| AD 2 Hippo | 17.9 | Control (Path) 4 | 32.1 |
| | | Temporal Ctx | |
| AD 3 Hippo | 6.0 | AD 1 Occipital Ctx | 17.7 |
| AD 4 Hippo | 4.1 | AD 2 Occipital Ctx | 0.0 |
| | | (Missing) | |
| AD 5 Hippo | 87.1 | AD 3 Occipital Ctx | 3.1 |
| AD 6 Hippo | 52.5 | AD 4 Occipital Ctx | 20.0 |
| Control 2 Hippo | 13.0 | AD 5 Occipital Ctx | 22.4 |
| Control 4 Hippo | 6.4 | AD 6 Occipital Ctx | 21.6 |
| Control (Path) 3 | 3.4 | Control 1 Occipital | 2.5 |
| Hippo | | Ctx | |
| AD 1 Temporal | 15.0 | Control 2 Occipital | 39.5 |
| Ctx | | Ctx | |
| AD 2 Temporal | 22.7 | Control 3 Occipital | 18.6 |
| Ctx | | Ctx | |
| AD 3 Temporal | 4.5 | Control 4 Occipital | 3.8 |
| Ctx | | Ctx | |
| AD 4 Temporal | 20.0 | Control (Path) 1 | 61.6 |
| Ctx | | Occipital Ctx | |
| AD 5 Inf Temporal | 100.0 | Control (Path) 2 | 10.4 |
| Ctx | | Occipital Ctx | |
| AD 5 Sup | 36.6 | Control (Path) 3 | 2.4 |
| Temporal Ctx | | Occipital Ctx | |
| AD 6 Inf Temporal | 46.7 | Control (Path) 4 | 14.1 |
| Ctx | | Occipital Ctx | |
| AD 6 Sup | 58.6 | Control 1 Parietal | 5.3 |
| Temporal Ctx | | Ctx | |
| Control 1 | 5.6 | Control 2 Parietal | 47.0 |
| Temporal Ctx | | Ctx | |
| Control 2 | 20.2 | Control 3 Parietal | 14.7 |
| Temporal Ctx | | Ctx | |
| Control 3 | 15.8 | Control (Path) 1 | 57.4 |
| Temporal Ctx | | Parietal Ctx | |
| Control 3 | 6.1 | Control (Path) 2 | 27.4 |
| Temporal Ctx | | Parietal Ctx | |
| Control (Path) 1 | 50.0 | Control (Path) 3 | 2.2 |
| Temporal Ctx | | Parietal Ctx | |
| Control (Path) 2 | 33.2 | Control (Path) 4 | 37.1 |
| Temporal Ctx | | Parietal Ctx | |

TABLE CKC
General_screening_panel_v1.4

| | Rel. Exp. | | Rel. Exp. |
|---|---|---|---|
| | (%) Ag3511, | | (%) Ag3511, |
| | Run | | Run |
| Tissue Name | 217240774 | Tissue Name | 217240774 |
| Adipose | 4.9 | Renal ca. TK-10 | 18.4 |
| Melanoma* | 15.9 | Bladder | 9.3 |
| Hs688(A).T | | | |
| Melanoma* | 12.8 | Gastric ca. (liver met.) | 24.3 |
| Hs688(B).T | | NCI-N87 | |
| Melanoma* M14 | 20.6 | Gastric ca. KATO III | 100.0 |
| Melanoma* | 9.1 | Colon ca. SW-948 | 4.7 |
| LOXIMVI | | | |
| Melanoma* SK- | 26.6 | Colon ca. SW480 | 63.7 |
| MEL-5 | | | |
| Squamous cell | 14.3 | Colon ca.*(SW480 | 26.1 |
| carcinoma SCC-4 | | met) SW620 | |
| Testis Pool | 6.3 | Colon ca. HT29 | 17.4 |
| Prostate ca.* (bone | 43.5 | Colon ca. HCT-116 | 24.3 |
| met) PC-3 | | | |
| Prostate Pool | 6.6 | Colon ca. CaCo-2 | 54.7 |
| Placenta | 0.7 | Colon cancer tissue | 6.5 |
| Uterus Pool | 4.4 | Colon ca. SW1116 | 4.8 |
| Ovarian ca. | 11.7 | Colon ca. Colo-205 | 2.0 |
| OVCAR-3 | | | |
| Ovarian ca. SK- | 45.1 | Colon ca. SW-48 | 4.0 |
| OV-3 | | | |
| Ovarian ca. | 17.9 | Colon Pool | 13.0 |
| OVCAR-4 | | | |
| Ovarian ca. | 26.1 | Small Intestine Pool | 14.1 |
| OVCAR-5 | | | |
| Ovarian ca. | 12.5 | Stomach Pool | 7.2 |
| IGROV-1 | | | |
| Ovarian ca. | 10.2 | Bone Marrow Pool | 6.5 |
| OVCAR-8 | | | |
| Ovary | 7.5 | Fetal Heart | 73.2 |
| Breast ca. MCF-7 | 9.7 | Heart Pool | 19.1 |
| Breast ca. MDA- | 47.0 | Lymph Node Pool | 14.5 |
| MB-231 | | | |
| Breast ca. BT 549 | 58.2 | Fetal Skeletal Muscle | 34.9 |
| Breast ca. T47D | 44.1 | Skeletal Muscle Pool | 45.1 |
| Breast ca. MDA-N | 12.9 | Spleen Pool | 9.8 |
| Breast Pool | 15.2 | Thymus Pool | 13.0 |
| Trachea | 7.1 | CNS cancer | 2.6 |
| | | (glio/astro) U87-MG | |
| Lung | 3.8 | CNS cancer | 33.4 |
| | | (glio/astro) U-118-MG | |
| Fetal Lung | 27.5 | CNS cancer | 8.5 |
| | | (neuro; met) SK-N-AS | |
| Lung ca. NCI-N417 | 2.5 | CNS cancer (astro) | 11.6 |
| | | SF-539 | |
| Lung ca. LX-1 | 24.0 | CNS cancer (astro) | 31.6 |
| | | SNB-75 | |
| Lung ca. NCI-H146 | 3.2 | CNS cancer (glio) | 12.1 |
| | | SNB-19 | |
| Lung ca. SHP-77 | 13.0 | CNS cancer (glio) SF- | 40.9 |
| | | 295 | |
| Lung ca. A549 | 18.9 | Brain (Amygdala) | 4.7 |
| | | Pool | |
| Lung ca. NCI-H526 | 20.6 | Brain (cerebellum) | 12.0 |
| Lung ca. NCI-H23 | 23.5 | Brain (fetal) | 10.7 |
| Lung ca. NCI-H460 | 10.5 | Brain (Hippocampus) | 3.8 |
| | | Pool | |
| Lung ca. HOP-62 | 10.2 | Cerebral Cortex Pool | 7.5 |
| Lung ca. NCI-H522 | 21.8 | Brain (Substantia | 3.2 |
| | | nigra) Pool | |
| Liver | 1.6 | Brain (Thalamus) Pool | 8.0 |
| Fetal Liver | 10.9 | Brain (whole) | 5.0 |
| Liver ca. HepG2 | 7.2 | Spinal Cord Pool | 3.8 |
| Kidney Pool | 16.3 | Adrenal Gland | 5.0 |
| Fetal Kidney | 16.3 | Pituitary gland Pool | 3.0 |
| Renal ca. 786-0 | 11.3 | Salivary Gland | 3.1 |
| Renal ca. A498 | 4.2 | Thyroid (female) | 3.1 |
| Renal ca. ACHN | 10.2 | Pancreatic ca. | 25.0 |
| | | CAPAN2 | |
| Renal ca. UO-31 | 17.1 | Pancreas Pool | 13.0 |

TABLE CKD
Panel 4D

| | Rel. Exp. (%) | | Rel. Exp. (%) |
|---|---|---|---|
| | Ag3511, Run | | Ag3511, Run |
| Tissue Name | 166407112 | Tissue Name | 166407112 |
| Secondary Th1 act | 26.6 | HUVEC IL-1beta | 14.8 |
| Secondary Th2 act | 31.6 | HUVEC IFN gamma | 16.8 |
| Secondary Tr1 act | 33.7 | HUVEC TNF alpha + | 9.7 |
| | | IFN gamma | |
| Secondary Th1 rest | 22.8 | HUVEC TNF alpha + | 9.1 |
| | | IL4 | |
| Secondary Th2 rest | 17.2 | HUVEC IL-11 | 9.9 |
| Secondary Tr1 rest | 20.3 | Lung Microvascular EC | 13.2 |
| | | none | |
| Primary Th1 act | 10.4 | Lung Microvascular EC | 8.3 |
| | | TNF alpha + IL-1beta | |
| Primary Th2 act | 17.2 | Microvascular Dermal | 22.7 |
| | | EC none | |
| Primary Tr1 act | 25.7 | Microsvasular Dermal | 11.9 |
| | | EC TNF alpha + IL-1beta | |
| Primary Th1 rest | 57.4 | Bronchial epithelium | 8.0 |
| | | TNF alpha + IL1beta | |
| Primary Th2 rest | 28.1 | Small airway epithelium | 3.7 |
| | | none | |
| Primary Tr1 rest | 15.6 | Small airway epithelium | 24.3 |
| | | TNF alpha + IL-1beta | |
| CD45RA CD4 | 11.0 | Coronery artery SMC rest | 11.9 |
| lymphocyte act | | | |
| CD45RO CD4 | 28.1 | Coronery artery SMC | 7.6 |
| lymphocyte act | | TNF alpha + IL-1beta | |
| CD8 lymphocyte act | 19.2 | Astrocytes rest | 10.6 |
| Secondary CD8 | 15.3 | Astrocytes TNF alpha + | 12.4 |
| lymphocyte rest | | IL-1beta | |
| Secondary CD8 | 20.3 | KU-812 (Basophil) rest | 20.2 |
| lymphocyte act | | | |
| CD4 lymphocyte none | 8.4 | KU-812 (Basophil) | 46.7 |
| | | PMA/ionomycin | |
| 2ry Th1/Th2/Tr1_anti- | 24.8 | CCD1106 | 12.6 |
| CD95 CH11 | | (Keratinocytes) none | |
| LAK cells rest | 12.2 | CCD1106 | 53.2 |
| | | (Keratinocytes) | |
| | | TNF alpha + IL-1beta | |
| LAK cells IL-2 | 49.3 | Liver cirrhosis | 7.7 |
| LAK cells IL-2 + IL-12 | 27.5 | Lupus kidney | 9.7 |
| LAK cells IL-2 + IFN | 47.6 | NCI-H292 none | 59.5 |
| gamma | | | |
| LAK cells IL-2 + IL-18 | 35.4 | NCI-H292 IL-4 | 100.0 |
| LAK cells | 3.6 | NCI-H292 IL-9 | 69.7 |
| PMA/ionomycin | | | |
| NK Cells IL-2 rest | 46.0 | NCI-H292 IL-13 | 46.7 |
| Two Way MLR 3 day | 24.7 | NCI-H292 IFN gamma | 36.6 |
| Two Way MLR 5 day | 22.1 | HPAEC none | 11.8 |
| Two Way MLR 7 day | 13.9 | HPAEC TNF alpha + IL- | 12.9 |
| | | 1beta | |
| PBMC rest | 13.0 | Lung fibroblast none | 13.3 |
| PBMC PWM | 17.3 | Lung fibroblast TNF | 31.9 |
| | | alpha + IL-1beta | |
| PBMC PHA-L | 11.7 | Lung fibroblast IL-4 | 9.6 |
| Ramos (B cell) none | 19.8 | Lung fibroblast IL-9 | 7.4 |
| Ramos (B cell) | 18.2 | Lung fibroblast IL-13 | 6.3 |
| ionomycin | | | |
| B lymphocytes PWM | 37.1 | Lung fibroblast IFN | 10.4 |
| | | gamma | |
| B lymphocytes CD40L | 34.6 | Dermal fibroblast | 23.7 |
| and IL-4 | | CCD1070 rest | |
| EOL-1 dbcAMP | 14.4 | Dermal fibroblast | 75.8 |
| | | CCD1070 TNF alpha | |
| EOL-1 dbcAMP | 21.6 | Dermal fibroblast | 18.7 |
| PMA/ionomycin | | CCD1070 IL-1beta | |
| Dendritic cells none | 10.8 | Dermal fibroblast IFN | 5.6 |
| | | gamma | |
| Dendritic cells LPS | 13.5 | Dermal fibroblast IL-4 | 11.3 |
| Dendritic cells anti- | 14.4 | IBD Colitis 2 | 5.3 |
| CD40 | | | |
| Monocytes rest | 11.6 | IBD Crohn's | 6.0 |
| Monocytes LPS | 7.6 | Colon | 52.9 |
| Macrophages rest | 20.3 | Lung | 9.7 |
| Macrophages LPS | 15.0 | Thymus | 16.3 |
| HUVEC none | 16.8 | Kidney | 19.9 |
| HUVEC starved | 29.9 | | |

CNS_neurodegeneration_v1.0 Summary: Ag3511 This panel confirms the expression of CG59708-01 gene at low levels in the brain in an independent group of individuals. However, no differential expression of this gene was detected between Alzheimer's diseased postmortem brains and those of non-demented controls in this experiment. However, as seen in panel 1.4, this gene is expressed at low levels throughout the CNS, including in amygdala, substantia nigra, thalamus, cerebellum, cerebral cortex, and spinal cord. Therefore, this gene may play a role in other central nervous system disorders such as Parkinson's disease, epilepsy, multiple sclerosis, schizophrenia and depression.

General_screening_panel_v1.4 Summary: Ag3511 Highest expression of the CG59708-01 is detected in a gastric cancer cell line sample (CT=27.1). Thus, expression of this gene can be used to distinguish this sample from other samples in this panel. In addition, high levels of expression of this gene are associated with breast cancer, ovarian cancer, and gastric cancer cell lines. Therefore, therapeutic modulation of the activity of this gene or its protein product might be beneficial in the treatment of these cancers.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at moderate to low levels in all regions of the CNS examined. Please see Panel CNS_neurodegeneration_v1.0 for discussion of utility of this gene in the central nervous system.

Panel 4D Summary: Ag3511 Highest expression of the CG59708-01 gene is detected in a IL-4 treated NCI-H292 sample (CT=26.4). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CL. CG59559-01: CPSase-Related

Expression of gene CG59559-01 was assessed using the primer-probe set Ag3469, described in Table CLA. Results of the RTQ-PCR runs are shown in Tables CLB, CLC and CLD.

Table CLA. Probe Name Ag3469

TABLE CLA
Probe Name Ag3469

| | | | Start | SEQ ID |
|---|---|---|---|---|
| Primers | Sequences | Length | Position | NO: |
| Forward | 5′-tccagtcggatcaattctattg-3′ | 22 | 1213 | 677 |
| Probe | TET-5′-attcagatgtcccctcatcagcccat-3′-TAMRA | 26 | 1237 | 678 |
| Reverse | 5′-aattgtcttcgacgaagaaacc-3′ | 22 | 1266 | 679 |

TABLE CLB
CNS_neurodegeneration_v1.0

| | | | Rel. Exp. |
|---|---|---|---|
| | Rel. Exp. (%) | | (%) Ag3469, |
| | Ag3469, Run | | Run |
| Tissue Name | 210376662 | Tissue Name | 210376662 |
| AD 1 Hippo | 23.5 | Control (Path) 3 | 13.9 |
| | | Temporal Ctx | |
| AD 2 Hippo | 31.0 | Control (Path) 4 | 35.6 |
| | | Temporal Ctx | |
| AD 3 Hippo | 17.1 | AD 1 Occipital Ctx | 8.1 |
| AD 4 Hippo | 23.0 | AD 2 Occipital Ctx | 0.0 |
| | | (Missing) | |
| AD 5 Hippo | 21.0 | AD 3 Occipital Ctx | 2.0 |
| AD 6 Hippo | 69.3 | AD 4 Occipital Ctx | 25.9 |
| Control 2 Hippo | 56.6 | AD 5 Occipital Ctx | 31.9 |
| Control 4 Hippo | 52.5 | AD 6 Occipital Ctx | 10.4 |
| Control (Path) 3 | 7.5 | Control 1 Occipital | 3.3 |
| Hippo | | Ctx | |
| AD 1 Temporal | 39.8 | Control 2 Occipital | 42.6 |
| Ctx | | Ctx | |
| AD 2 Temporal | 35.8 | Control 3 Occipital | 10.7 |
| Ctx | | Ctx | |
| AD 3 Temporal | 12.2 | Control 4 Occipital | 17.6 |
| Ctx | | Ctx | |
| AD 4 Temporal | 30.1 | Control (Path) 1 | 59.0 |
| Ctx | | Occipital Ctx | |
| AD 5 Inf Temporal | 37.6 | Control (Path) 2 | 14.6 |
| Ctx | | Occipital Ctx | |
| AD 5 Sup | 33.4 | Control (Path) 3 | 0.0 |
| Temporal Ctx | | Occipital Ctx | |
| AD 6 Inf Temporal | 52.1 | Control (Path) 4 | 10.6 |
| Ctx | | Occipital Ctx | |
| AD 6 Sup | 31.0 | Control 1 Parietal | 9.1 |
| Temporal Ctx | | Ctx | |
| Control 1 | 12.7 | Control 2 Parietal | 28.5 |
| Temporal Ctx | | Ctx | |
| Control 2 | 31.4 | Control 3 Parietal | 17.2 |
| Temporal Ctx | | Ctx | |
| Control 3 | 36.3 | Control (Path) 1 | 64.2 |
| Temporal Ctx | | Parietal Ctx | |
| Control 3 | 16.8 | Control (Path) 2 | 25.7 |
| Temporal Ctx | | Parietal Ctx | |
| Control (Path) 1 | 100.0 | Control (Path) 3 | 1.3 |
| Temporal Ctx | | Parietal Ctx | |
| Control (Path) 2 | 62.4 | Control (Path) 4 | 55.9 |
| Temporal Ctx | | Parietal Ctx | |

TABLE CLC
General_screening_panel_v1.4

| | Rel. Exp. | | Rel. Exp. |
|---|---|---|---|
| | (%) Ag3469, | | (%) Ag3469, |
| | Run | | Run |
| Tissue Name | 217119419 | Tissue Name | 217119419 |
| Adipose | 3.8 | Renal ca. TK-10 | 7.1 |
| Melanoma* | 9.2 | Bladder | 5.7 |
| Hs688(A).T | | | |
| Melanoma* | 4.7 | Gastric ca. (liver met.) | 4.9 |
| Hs688(B).T | | NCI-N87 | |
| Melanorna* M14 | 0.1 | Gastric ca. KATO III | 1.0 |
| Melanoma* | 0.8 | Colon ca. SW-948 | 0.8 |
| LOXIMVI | | | |
| Melanoma* SK- | 2.3 | Colon ca. SW480 | 0.0 |
| MEL-5 | | | |
| Squamous cell | 1.9 | Colon ca.* (SW480 | 5.1 |
| carcinoma SCC-4 | | met) SW620 | |
| Testis Pool | 5.1 | Colon ca. HT29 | 8.9 |
| Prostate ca.* (bone | 3.6 | Colon ca. HCT-116 | 0.7 |
| met) PC-3 | | | |
| Prostate Pool | 1.8 | Colon ca. CaCo-2 | 8.1 |
| Placenta | 1.4 | Colon cancer tissue | 8.2 |
| Uterus Pool | 2.7 | Colon ca. SW1116 | 0.3 |
| Ovarian ca. | 7.9 | Colon ca. Colo-205 | 1.6 |
| OVCAR-3 | | | |
| Ovarian ca. SK- | 11.3 | Colon ca. SW-48 | 0.9 |
| OV-3 | | | |
| Ovarian ca. | 0.8 | Colon Pool | 10.4 |
| OVCAR-4 | | | |
| Ovarian ca. | 9.2 | Small Intestine Pool | 3.6 |
| OVCAR-5 | | | |
| Ovarian ca. | 5.5 | Stomach Pool | 2.7 |
| IGROV-1 | | | |
| Ovarian ca. | 2.0 | Bone Marrow Pool | 6.3 |
| OVCAR-8 | | | |
| Ovary | 16.7 | Fetal Heart | 0.9 |
| Breast ca. MCF-7 | 31.6 | Heart Pool | 3.8 |
| Breast ca. MDA- | 4.0 | Lymph Node Pool | 11.9 |
| MB-231 | | | |
| Breast ca. BT 549 | 3.7 | Fetal Skeletal Muscle | 0.4 |
| Breast ca. T47D | 20.9 | Skeletal Muscle Pool | 0.5 |
| Breast ca. MDA-N | 0.1 | Spleen Pool | 10.7 |
| Breast Pool | 11.3 | Thymus Pool | 7.5 |
| Trachea | 3.8 | CNS cancer | 10.2 |
| | | (glio/astro) U87-MG | |
| Lung | 9.3 | CNS cancer | 1.3 |
| | | (glio/astro) U-118-MG | |
| Fetal Lung | 3.8 | CNS cancer | 0.1 |
| | | (neuro; met) SK-N-AS | |
| Lung ca. NCI-N417 | 0.0 | CNS cancer (astro) | 3.0 |
| | | SF-539 | |
| Lung ca. LX-1 | 13.8 | CNS cancer (astro) | 10.8 |
| | | SNB-75 | |
| Lung ca. NCI-H146 | 0.1 | CNS cancer (glio) | 4.9 |
| | | SNB-19 | |
| Lung ca. SHP-77 | 6.0 | CNS cancer (glio) SF- | 21.0 |
| | | 295 | |
| Lung ca. A549 | 100.0 | Brain (Amygdala) | 1.1 |
| | | Pool | |
| Lung ca. NCI-H526 | 0.0 | Brain (cerebellum) | 2.0 |
| Lung ca. NCI-H23 | 0.1 | Brain (fetal) | 1.2 |
| Lung ca. NCI-H460 | 8.9 | Brain (Hippocampus) | 1.1 |
| | | Pool | |
| Lung ca. HOP-62 | 45.4 | Cerabral Cortex Pool | 1.2 |
| Lung ca. NCI-H522 | 4.2 | Brain (Substantia | 1.3 |
| | | nigra) Pool | |
| Liver | 0.6 | Brain (Thalamus) Pool | 1.2 |
| Fetal Liver | 1.8 | Brain (whole) | 1.1 |
| Liver ca. HepG2 | 1.5 | Spinal Cord Pool | 1.9 |
| Kidney Pool | 8.8 | Adrenal Gland | 26.8 |
| Fetal Kidney | 3.7 | Pituitary gland Pool | 0.5 |
| Renal ca. 786-0 | 3.9 | Salivary Gland | 1.3 |
| Renal ca. A498 | 10.5 | Thyroid (female) | 0.7 |
| Renal ca. ACHN | 6.2 | Pancreatic ca. | 20.2 |
| | | CAPAN2 | |
| Renal ca. UO-31 | 6.3 | Pancreas Pool | 9.6 |

TABLE CLD
Panel 4.1D

| | Rel. Exp. (%) | | Rel. Exp. (%) |
|---|---|---|---|
| | Ag3469, Run | | Ag3469, Run |
| Tissue Name | 169839390 | Tissue Name | 169839390 |
| Secondary Th1 act | 19.2 | HUVEC IL-1beta | 1.3 |
| Secondary Th2 act | 25.2 | HUVEC IFN gamma | 6.2 |
| Secondary Tr1 act | 14.4 | HUVEC TNF alpha + | 1.3 |
| | | IFN gamma | |
| Secondary Th1 rest | 26.1 | HUVEC TNF alpha + | 1.1 |
| | | IL4 | |
| Secondary Th2 rest | 46.3 | HUVEC IL-11 | 1.1 |
| Secondary Tr1 rest | 36.6 | Lung Microvascular EC | 0.7 |
| | | none | |
| Primary Th1 act | 13.6 | Lung Microvascular EC | 0.4 |
| | | TNF alpha + IL-1beta | |
| Primary Th2 act | 27.7 | Microvascular Dermal | 0.1 |
| | | EC none | |
| Primary Tr1 act | 15.2 | Microsvasular Dermal | 0.0 |
| | | EC TNF alpha + IL-1beta | |
| Primary Th1 rest | 46.0 | Bronchial epithelium | 5.8 |
| | | TNF alpha + IL1beta | |
| Primary Th2 rest | 44.4 | Small airway epithelium | 2.0 |
| | | none | |
| Primary Tr1 rest | 45.1 | Small airway epithelium | 6.9 |
| | | TNF alpha + IL-1beta | |
| CD45RA CD4 | 25.2 | Coronery artery SMC rest | 3.1 |
| lymphocyte act | | | |
| CD45RO CD4 | 67.8 | Coronery artery SMC | 3.4 |
| lymphocyte act | | TNF alpha + IL-1beta | |
| CD8 lymphocyte act | 59.5 | Astrocytes rest | 1.4 |
| Secondary CD8 | 47.0 | Astrocytes TNF alpha + | 3.2 |
| lymphocyte rest | | IL-1beta | |
| Secondary CD8 | 38.4 | KU-812 (Basophil) rest | 3.0 |
| lymphocyte act | | | |
| CD4 lymphocyte none | 61.1 | KU-812 (Basophil) | 6.4 |
| | | PMA/ionomycin | |
| 2ry Th1/Th2/Tr1_anti- | 27.2 | CCD1106 | 3.8 |
| CD95 CH11 | | (Keratinocytes) none | |
| LAK cells rest | 50.7 | CCD1106 | 5.2 |
| | | (Keratinocytes) | |
| | | TNF alpha + IL-1beta | |
| LAK cells IL-2 | 36.9 | Liver cirrhosis | 3.0 |
| LAK cells IL-2 + IL-12 | 49.0 | NCI-H292 none | 5.0 |
| LAK cells IL-2 + IFN | 59.5 | NCI-H292 IL-4 | 5.5 |
| gamma | | | |
| LAK cells IL-2 + IL-18 | 58.2 | NCI-H292 IL-9 | 6.8 |
| LAK cells | 65.5 | NCI-H292 IL-13 | 6.7 |
| PMA/ionomycin | | | |
| NK Cells IL-2 rest | 47.0 | NCI-H292 IFN gamma | 8.0 |
| Two Way MLR 3 day | 54.0 | HPAEC none | 2.7 |
| Two Way MLR 5 day | 25.2 | HPAEC TNF alpha + IL- | 3.3 |
| | | 1beta | |
| Two Way MLR 7 day | 42.6 | Lung fibroblast none | 1.5 |
| PBMC rest | 23.5 | Lung fibroblast TNF | 1.1 |
| | | alpha + IL-1beta | |
| PBMC PWM | 37.4 | Lung fibroblast IL-4 | 0.7 |
| PBMC PHA-L | 59.5 | Lung fibroblast IL-9 | 3.1 |
| Ramos (B cell) none | 1.8 | Lung fibroblast IL-13 | 0.7 |
| Ramos (B cell) | 2.5 | Lung fibroblast IFN | 0.3 |
| ionomycin | | gamma | |
| B lymphocytes PWM | 26.8 | Dermal fibroblast | 7.7 |
| | | CCD1070 rest | |
| B lymphocytes CD40L | 100.0 | Dermal fibroblast | 36.9 |
| and IL-4 | | CCD1070 TNF alpha | |
| EOL-1 dbcAMP | 2.7 | Dermal fibroblast | 5.5 |
| | | CCD1070 IL-1beta | |
| EOL-1 dbcAMP | 0.5 | Dermal fibroblast IFN | 0.7 |
| PMA/ionomycin | | gamma | |
| Dendritic cells none | 10.4 | Dermal fibroblast IL-4 | 3.7 |
| Dendritic cells LPS | 3.2 | Dermal Fibroblasts rest | 0.2 |
| Dendritic cells anti- | 4.2 | Neutrophils TNFa + LPS | 0.8 |
| CD40 | | | |
| Monocytes rest | 1.7 | Neutrophils rest | 3.2 |
| Monocytes LPS | 2.6 | Colon | 4.4 |
| Macrophages rest | 5.6 | Lung | 5.0 |
| Macrophages LPS | 2.6 | Thymus | 6.9 |
| HUVEC none | 0.7 | Kidney | 2.0 |
| HUVEC starved | 1.6 | | |

CNS_neurodegeneration_v1.0 Summary: Ag3469 This panel confirms the expression of the CG59559-01 gene at low levels in the brain in an independent group of individuals. However, no differential expression of this gene was detected between Alzheimer's diseased postmortem brains and those of non-demented controls in this experiment. However, as seen in panel 1.4, this gene is expressed at low levels throughout the CNS, including in amygdala, substantia nigra, thalamus, cerebellum, cerebral cortex, and spinal cord. Therefore, this gene may play a role in other central nervous system disorders such as Parkinson's disease, epilepsy, multiple sclerosis, schizophrenia and depression.

General_screening_panel_v1.4 Summary: Ag3469 Highest expression of the CG59559-01 gene is detected in sample derived from a lung cancer cell line (CT=25.6). Thus, expression of this gene can be used to distinguish this sample from other samples used in this panel. Furthermore, significant expression of this gene is associated with pancreatic cancer, CNS cancer and breast cancer cell lines. Therefore, therapeutic modulation of the activity of this gene or its protein product might be beneficial in the treatment of these cancers.

Among tissues with metabolic function, this gene is expressed in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at low but significant levels in all regions of the CNS examined. Please see Panel CNS_neurodegeneration_v1.0 for discussion of utility of this gene in the central nervous system.

Panel 4.1D Summary: Ag3469 Highest expression of the CG59559-01 gene is detected in sample derived CD40L and IL-4 treated B lymphocytes (CT=27.2). Fur5hermore, this gene is expressed at significant levels in a wide range of cell types of significance in the immune response in health and disease. These cells include members of the T-cell, B-cell, endothelial cell, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CM. CG59669-01: Carbonyl Reductase

Expression of gene CG59669-01 was assessed using the primer-probe set Ag3505, described in Table CMA.

Table CMA. Probe Name Ag3505

TABLE CMA
Probe Name Ag3505

| | | | | SEQ ID |
|---|---|---|---|---|
| Primers | Sequences | Length | Start Position | NO: |
| Forward | 5′-ccgggtcccagaatctagt-3′ | 19 | 4 | 680 |
| Probe | TET-5′-cctacgccacggttttgaccacg-3′-TAMRA | 23 | 23 | 681 |
| Reverse | 5′-gacacacggaccacctgat-3′ | 19 | 74 | 682 |

CNS_neurodegeneration_v1.0 Summary: Ag3505 Expression of the CG59669-01 gene is low/undetectable (CTs>35) across all of the samples on this panel (data not shown).

General_screening_panel_v1.4 Summary: Ag3505 Results from one experiment with the CG59669-01 gene are not included. The amp plot indicates that there were experimental difficulties with this run.

Panel 4.1D Summary: Ag3505 Expression of the CG59669-01 gene is low/undetectable (CTs>35) across all of the samples on this panel due to a probable probe or chemistry failure (data not shown).

Panel 5 Islet Summary: Ag3505 Expression of the CG59669-01 gene is low/undetectable (CTs>35) across all of the samples on this panel (data not shown).

CN. CG59679-01: Carbonyl Reductase

Expression of gene CG59679-01 was assessed using the primer-probe set Ag3507, described in Table CNA.

Table CNA. Probe Name Ag3507

TABLE CNA
Probe Name Ag3507

| | | | Start | SEQ ID |
|---|---|---|---|---|
| Primers | Sequences | Length | Position | NO: |
| Forward | 5′-gactggagctaataagggcatt-3′ | 22 | 130 | 683 |
| Probe | TET-5′-tcgtgacctgtgtcagcaattctcag-3′-TAMRA | 26 | 166 | 684 |
| Reverse | 5′-gtgcagtgagcaccacatc-3′ | 19 | 194 | 685 |

CNS_neurodegeneration_v1.0 Summary: Ag3507 Expression of the CG59679-01 gene is low/undetectable (CTs>35) across all of the samples on this panel (data not shown).

General_screening_panel_v1.4 Summary: Ag3507 Expression of the CG59679-01 gene is low/undetectable (CTs>35) across all of the samples on this panel (data not shown).

Panel 4.1D Summary: Ag3507 Expression of the CG59679-01 gene is low/undetectable (CTs>35) across all of the samples on this panel (data not shown). The data suggest that there may have been experimental difficulties with this run.

Panel 5 Islet Summary: Ag3507 Expression of CG59679-01 gene is low/undetectable (CTs>35) across all of the samples on this panel (data not shown).

CO. CG59644-01: Putative Protein Phosphatase

Expression of gene CG59644-01 was assessed using the primer-probe set Ag3503, described in Table COA. Results of the RTQ-PCR runs are shown in Tables COB, COC and COD.

Table COA. Probe Name Ag3503

TABLE COA
Probe Name Ag3503

| | | | Start | SEQ ID |
|---|---|---|---|---|
| Primers | Sequences | Length | Position | NO: |
| Forward | 5′-tcgtacctagtaatcccattgg-3′ | 22 | 410 | 763 |
| Probe | TET-5′-ccacaagctactgtgagttgctgcaa-3′-TAMRA | 26 | 440 | 764 |
| Reverse | 5′-ctaccgagcaaagggactttat-3′ | 22 | 467 | 765 |

TABLE COB
CNS_neurodegeneration_v1.0

| | | | Rel. Exp. |
|---|---|---|---|
| | Rel. Exp. (%) | | (%) Ag3503, |
| | Ag3503, Run | | Run |
| Tissue Name | 210938272 | Tissue Name | 210938272 |
| AD 1 Hippo | 22.1 | Control (Path) 3 | 5.7 |
| | | Temporal Ctx | |
| AD 2 Hippo | 29.1 | Control (Path) 4 | 25.3 |
| | | Temporal Ctx | |
| AD 3 Hippo | 10.8 | AD 1 Occipital | 21.8 |
| | | Ctx | |
| AD 4 Hippo | 9.4 | AD 2 Occipital | 0.0 |
| | | Ctx (Missing) | |
| AD 5 hippo | 100.0 | AD 3 Occipital | 8.9 |
| | | Ctx | |
| AD 6 Hippo | 80.7 | AD 4 Occipital | 22.8 |
| | | Ctx | |
| Control 2 Hippo | 27.0 | AD 5 Occipital | 26.8 |
| | | Ctx | |
| Control 4 Hippo | 11.4 | AD 6 Occipital | 56.6 |
| | | Ctx | |
| Control (Path) 3 | 11.3 | Control 1 Occipital | 5.6 |
| Hippo | | Ctx | |
| AD 1 Temporal Ctx | 18.9 | Control 2 Occipital | 93.3 |
| | | Ctx | |
| AD 2 Temporal Ctx | 29.5 | Control 3 Occipital | 15.8 |
| | | Ctx | |
| AD 3 Temporal Ctx | 7.2 | Control 4 Occipital | 6.1 |
| | | Ctx | |
| AD 4 Temporal Ctx | 19.3 | Control (Path) 1 | 80.1 |
| | | Occipital Ctx | |
| AD 5 Inf Temporal | 73.2 | Control (Path) 2 | 7.5 |
| Ctx | | Occipital Ctx | |
| AD 5 Sup Temporal | 49.0 | Control (Path) 3 | 5.9 |
| Ctx | | Occipital Ctx | |
| AD 6 Inf Temporal | 68.8 | Control (Path) 4 | 14.5 |
| Ctx | | Occipital Ctx | |
| AD 6 Sup Temporal | 66.0 | Control 1 Parietal | 7.6 |
| Ctx | | Ctx | |
| Control 1 Temporal | 4.4 | Control 2 Parietal | 29.1 |
| Ctx | | Ctx | |
| Control 2 Temporal | 53.2 | Control 3 Parietal | 26.6 |
| Ctx | | Ctx | |
| Control 3 Temporal | 9.7 | Control (Path) 1 | 95.9 |
| Ctx | | Parietal Ctx | |
| Control 4 Temporal | 11.3 | Control (Path) 2 | 22.5 |
| Ctx | | Parietal Ctx | |
| Control (Path) 1 | 48.0 | Control (Path) 3 | 6.8 |
| Temporal Ctx | | Parietal Ctx | |
| Control (Path) 2 | 22.2 | Control (Path) 4 | 49.7 |
| Temporal Ctx | | Parietal Ctx | |

TABLE COC
General_screening_panel_v1.4

| | Rel. Exp. | | Rel. Exp. |
|---|---|---|---|
| | (%) Ag3503, | | (%) Ag3503, |
| | Run | | Run |
| Tissue Name | 217131685 | Tissue Name | 217131685 |
| Adipose | 12.9 | Renal ca. TK-10 | 26.8 |
| Melanoma* | 7.7 | Bladder | 22.1 |
| Hs688(A).T | | | |
| Melanoma* | 11.0 | Gastric ca. (liver met.) | 63.7 |
| Hs688(B).T | | NCI-N87 | |
| Melanoma* M14 | 25.0 | Gastric ca. KATO III | 66.0 |
| Melanoma* | 25.9 | Colon ca. SW-948 | 16.4 |
| LOXIMVI | | | |
| Melanoma* SK- | 69.3 | Colon ca. SW480 | 37.4 |
| MEL-5 | | | |
| Squamous cell | 18.9 | Colon ca.* (SW480 | 24.8 |
| carcinoma SCC-4 | | met) SW620 | |
| Testis Pool | 13.0 | Colon ca. HT29 | 13.7 |
| Prostate ca.* (bone | 51.8 | Colon ca. HCT-116 | 60.3 |
| met) PC-3 | | | |
| Prostate Pool | 6.9 | Colon ca. CaCo-2 | 25.7 |
| Placenta | 7.1 | Colon cancer tissue | 34.9 |
| Uterus Pool | 5.9 | Colon ca. SW1116 | 8.2 |
| Ovarian ca. | 14.0 | Colon ca. Colo-205 | 9.2 |
| OVCAR-3 | | | |
| Ovarian ca. SK- | 90.8 | Colon ca. SW-48 | 5.8 |
| OV-3 | | | |
| Ovarian ca. | 12.5 | Colon Pool | 17.4 |
| OVCAR-4 | | | |
| Ovarian ca. | 34.2 | Small Intestine Pool | 15.7 |
| OVCAR-5 | | | |
| Ovarian ca. | 33.0 | Stomach Pool | 6.6 |
| IGROV-1 | | | |
| Ovarian ca. | 17.6 | Bone Marrow Pool | 6.4 |
| OVCAR-8 | | | |
| Ovary | 7.8 | Fetal Heart | 12.1 |
| Breast ca. MCF-7 | 15.9 | Heart Pool | 17.7 |
| Breast ca. MDA- | 55.1 | Lymph Node Pool | 17.6 |
| MB-231 | | | |
| Breast ca. BT 549 | 19.8 | Fetal Skeletal Muscle | 4.8 |
| Breast ca. T47D | 66.9 | Skeletal Muscle Pool | 100.0 |
| Breast ca. MDA-N | 12.2 | Spleen Pool | 12.5 |
| Breast Pool | 17.6 | Thymus Pool | 14.2 |
| Trachea | 28.7 | CNS cancer | 42.9 |
| | | (glio/astro) U87-MG | |
| Lung | 3.0 | CNS cancer | 55.1 |
| | | (glio/astro) U-118-MG | |
| Fetal Lung | 18.8 | CNS cancer | 30.6 |
| | | (neuro; met) SK-N-AS | |
| Lung ca. NCI-N417 | 9.1 | CNS cancer (astro) | 12.2 |
| | | SF-539 | |
| Lung ca. LX-1 | 41.2 | CNS cancer (astro) | 22.4 |
| | | SNB-75 | |
| Lung ca. NCI-H146 | 7.0 | CNS cancer (glio) | 36.3 |
| | | SNB-19 | |
| Lung ca. SHP-77 | 26.8 | CNS cancer (glio) SF- | 50.7 |
| | | 295 | |
| Lung ca. A549 | 24.8 | Brain (Amygdala) | 12.4 |
| | | Pool | |
| Lung ca. NCI-H526 | 8.0 | Brain (cerebellum) | 14.6 |
| Lung ca. NCI-H23 | 26.6 | Brain (fetal) | 10.2 |
| Lung ca. NCI-H460 | 29.7 | Brain (Hippocampus) | 13.8 |
| | | Pool | |
| Lung ca. HOP-62 | 8.0 | Cerebral Cortex Pool | 17.2 |
| Lung ca. NCI-H522 | 19.9 | Brain (Substantia | 19.1 |
| | | nigra) Pool | |
| Liver | 2.9 | Brain (Thalamus) Pool | 18.4 |
| Fetal Liver | 10.4 | Brain (whole) | 15.7 |
| Liver ca. HepG2 | 14.2 | Spinal Cord Pool | 10.9 |
| Kidney Pool | 23.8 | Adrenal Gland | 25.7 |
| Fetal Kidney | 10.4 | Pituitary gland Pool | 3.9 |
| Renal ca. 786-0 | 12.3 | Salivary Gland | 11.1 |
| Renal ca. A498 | 3.7 | Thyroid (female) | 4.4 |
| Renal ca. ACHN | 27.9 | Pancreatic ca. | 15.0 |
| | | CAPAN2 | |
| Renal ca. UO-31 | 12.0 | Pancreas Pool | 17.1 |

TABLE COD
Panel 4D

| | Rel. Exp. (%) | | Rel. Exp. (%) |
|---|---|---|---|
| | Ag3503, Run | | Ag3503, Run |
| Tissue Name | 166441943 | Tissue Name | 166441943 |
| Secondary Th1 act | 54.3 | HUVEC IL-1beta | 20.4 |
| Secondary Th2 act | 52.5 | HUVEC IFN gamma | 14.6 |
| Secondary Tr1 act | 61.1 | HUVEC TNF alpha + | 16.0 |
| | | IFN gamma | |
| Secondary Th1 rest | 34.6 | HUVEC TNF alpha + | 24.8 |
| | | IL4 | |
| Secondary Th2 rest | 20.0 | HUVEC IL-11 | 6.6 |
| Secondary Tr1 rest | 23.0 | Lung Microvascular EC | 12.9 |
| | | none | |
| Primary Th1 act | 39.5 | Lung Microvascular EC | 17.4 |
| | | TNF alpha + IL-1beta | |
| Primary Th2 act | 59.9 | Microvascular Dermal | 21.6 |
| | | EC none | |
| Primary Tr1 act | 92.7 | Microsvasular Dermal | 20.2 |
| | | EC TNF alpha + IL-1beta | |
| Primary Th1 rest | 94.6 | Bronchial epithelium | 17.7 |
| | | TNF alpha + IL1beta | |
| Primary Th2 rest | 31.4 | Small airway epithelium | 7.9 |
| | | none | |
| Primary Tr1 rest | 32.8 | Small airway epithelium | 53.6 |
| | | TNF alpha + IL-1beta | |
| CD45RA CD4 | 44.1 | Coronery artery SMC rest | 7.7 |
| lymphocyte act | | | |
| CD45RO CD4 | 65.5 | Coronery artery SMC | 8.3 |
| lymphocyte act | | TNF alpha + IL-1beta | |
| CD8 lymphocyte act | 68.3 | Astrocytes rest | 11.7 |
| Secondary CD8 | 68.8 | Astrocytes TNF alpha + | 21.9 |
| lymphocyte rest | | IL-1beta | |
| Secondary CD8 | 40.1 | KU-812 (Basophil) rest | 12.1 |
| lymphocyte act | | | |
| CD4 lymphocyte none | 20.9 | KU-812 (Basophil) | 42.6 |
| | | PMA/ionomycin | |
| 2ry Th1/Th2/Tr1_anti- | 40.9 | CCD1106 | 21.0 |
| CD95 CH11 | | (Keratinocytes) none | |
| LAK cells rest | 22.7 | CCD1106 | 72.2 |
| | | (Keratinocytes) | |
| | | TNF alpha + IL-1beta | |
| LAK cells IL-2 | 46.7 | Liver cirrhosis | 12.8 |
| LAK cells IL-2 + IL-12 | 38.2 | Lupus Kidney | 16.7 |
| LAK cells IL-2 + IFN | 68.8 | NCI-H292 none | 33.0 |
| gamma | | | |
| LAK cells IL-2 + IL-18 | 57.8 | NCI-H292 IL-4 | 35.1 |
| LAK cells | 100.0 | NCI-H292 IL-9 | 35.4 |
| PMA/ionomycin | | | |
| NK Cells IL-2 rest | 32.8 | NCI-H292 IL-13 | 24.8 |
| Two Way MLR 3 day | 42.9 | NCI-H292 IFN gamma | 22.5 |
| Two Way MLR 5 day | 39.8 | HPAEC none | 11.2 |
| Two Way MLR 7 day | 33.7 | HPAEC TNF alpha + IL- | 27.9 |
| | | 1beta | |
| PBMC rest | 22.5 | Lung fibroblast none | 10.6 |
| PBMC PWM | 54.0 | Lung fibroblast TNF | 25.5 |
| | | alpha + IL-1beta | |
| PBMC PHA-L | 20.6 | Lung fibroblast IL-4 | 11.6 |
| Ramos (B cell) none | 56.3 | Lung fibroblast IL-9 | 5.4 |
| Ramos (B cell) | 38.4 | Lung fibroblast IL-13 | 8.5 |
| ionomycin | | | |
| B lymphocytes PWM | 44.8 | Lung fibroblast IFN | 11.7 |
| | | gamma | |
| B lymphocytes CD40L | 42.9 | Dermal fibroblast | 16.4 |
| and IL-4 | | CCD1070 rest | |
| EOL-1 dbcAMP | 18.2 | Dermal fibroblast | 56.3 |
| | | CCD1070 TNF alpha | |
| EOL-1 dbcAMP | 30.4 | Dermal fibroblast | 14.4 |
| PMA/ionomycin | | CCD1070 IL-1beta | |
| Dendritic cells none | 31.2 | Dermal fibroblast IFN | 14.5 |
| | | gamma | |
| Dendritic cells LPS | 40.9 | Dermal fibroblast IL-4 | 29.1 |
| Dendritic cells anti- | 31.2 | IBD Colitis 2 | 13.7 |
| CD40 | | | |
| Monocytes rest | 50.0 | IBD Crohn's | 11.6 |
| Monocytes LPS | 31.0 | Colon | 70.7 |
| Macrophages rest | 26.4 | Lung | 17.1 |
| Macrophages LPS | 28.9 | Thymus | 29.3 |
| HUVEC none | 22.2 | Kidney | 40.1 |
| HUVEC starved | 25.3 | | |

CNS_neurodegeneration_v1.0 Summary: Ag3503 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3503 Expression of the CG59644-01 gene is highest in adult skeletal muscle (CT=25.5). Interestingly, expression of this gene is much lower in fetal skeletal muscle (CT=29.9), suggesting that expression of this gene may be used to distinguish adult and fetal skeletal muscle.

The CG59644-01 gene encodes a protein with homology to protein phosphatases. This gene is expressed at high to moderate levels in the majority of samples on this panel. However, expression of this gene appears to be higher in cancer cell lines when compared to normal adult tissues. This observation is consistent with the potential role for this gene product in cell survival and proliferation.

In addition, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 4D Summary: Ag3503 This gene is expressed at high to moderate levels in a wide range of cell types of significance in the immune response in health and disease. These cells include T cells, B cells, endothelial cells, macrophages, monocytes, dendritic cells, basophils, eosinophils and peripheral blood mononuclear cells, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis., and osteoarthritis.

CP. CG59662-01: Cyclophilin

Expression of gene CG59662-01 was assessed using the primer-probe set Ag3504, described in Table CPA. Results of the RTQ-PCR runs are shown in Tables CPB and CPC.

Table CPA. Probe Name Ag3504

TABLE CPA
Probe Name Ag3504

| | | | Start | SEQ ID |
|---|---|---|---|---|
| Primers | Sequences | Length | Position | NO: |
| Forward | 5′-ggtcaaccacaccatgttctt-3′ | 21 | 22 | 686 |
| Probe | TET-5′-cttggaccacgtctcctttgagctg-3′-TAMRA | 25 | 67 | 687 |
| Reverse | 5′-tctttggaaacttttctgcaaa-3′ | 22 | 92 | 688 |

Table CPB. General_screening_panel_v1.4

TABLE CPB
General_screening_panel_v1.4

| | Ref. Exp. (%) | | Rel. Exp. (%) |
|---|---|---|---|
| | Ag3504, Run | | Ag3504, Run |
| Tissue Name | 217236170 | Tissue Name | 217236170 |
| Adipose | 0.5 | Renal ca. TK-10 | 0.4 |
| Melanoma* | 1.0 | Bladder | 0.6 |
| Hs688(A).T | | | |
| Melanoma* | 0.4 | Gastric ca. (liver met.) | 4.8 |
| Hs688(B).T | | NCI-N87 | |
| Melanoma* M14 | 100.0 | Gastric ca. KATO III | 14.2 |
| Melanoma* | 3.9 | Colon ca. SW-948 | 2.3 |
| LOXIMVI | | | |
| Melanoma* SK- | 4.9 | Colon ca. SW480 | 12.9 |
| MEL-5 | | | |
| Squamous Cell | 47.6 | Colon ca.* (SW480 | 0.3 |
| carcinoma SCC-4 | | met) SW620 | |
| Testis Pool | 2.7 | Colon ca. HT29 | 3.5 |
| Prostate ca.* (bone | 3.1 | Colon ca. HCT-116 | 1.1 |
| met) PC-3 | | | |
| Prostate Pool | 5.7 | Colon ca. CaCo-2 | 11.6 |
| Placenta | 5.0 | Colon cancer tissue | 7.6 |
| Uterus Pool | 2.2 | Colon ca. SW1116 | 1.5 |
| Ovarian ca. | 20.6 | Colon ca. Colo-205 | 11.7 |
| OVCAR-3 | | | |
| Ovarian ca. SK- | 6.3 | Colon ca. SW-48 | 6.2 |
| OV-3 | | | |
| Ovarian ca. | 1.5 | Colon Pool | 4.7 |
| OVCAR-4 | | | |
| Ovarian ca. | 10.2 | Small Intestine Pool | 2.5 |
| OVCAR-5 | | | |
| Ovarian ca. | 0.6 | Stomach Pool | 5.8 |
| IGROV-1 | | | |
| Ovarian ca. | 0.7 | Bone Marrow Pool | 1.2 |
| OVCAR-8 | | | |
| Ovary | 2.2 | Fetal Heart | 3.3 |
| Breast ca. MCF-7 | 2.9 | Heart Pool | 2.2 |
| Breast ca. MDA- | 4.0 | Lymph Node Pool | 11.8 |
| MB-231 | | | |
| Breast ca. BT 549 | 5.6 | Fetal Skeletal Muscle | 0.9 |
| Breast ca. T47D | 15.0 | Skeletal Muscle Pool | 32.3 |
| Breast ca. MDA-N | 17.7 | Spleen Pool | 2.0 |
| Breast Pool | 0.9 | Thymus Pool | 9.3 |
| Trachea | 2.0 | CNS cancer | 1.0 |
| | | (glio/astro) U87-MG | |
| Lung | 16.3 | CNS cancer | 3.3 |
| | | (glio/astro) U-118-MG | |
| Fetal Lung | 42.0 | CNS cancer | 6.2 |
| | | (neuro;met) SK-N-AS | |
| Lung ca. NCI-N417 | 3.6 | CNS cancer (astro) SF- | 2.3 |
| | | 539 | |
| Lung ca. LX-1 | 0.7 | CNS cancer (astro) | 12.7 |
| | | SNB-75 | |
| Lung ca. NCI-H146 | 5.1 | CNS cancer (glio) | 4.1 |
| | | SNB-19 | |
| Lung ca. SHP-77 | 17.6 | CNS cancer (glio) SF- | 0.7 |
| | | 295 | |
| Lung ca. A549 | 19.9 | Brain (Amygdala) Pool | 3.3 |
| Lung ca. NCI-H526 | 4.3 | Brain (cerebellum) | 2.3 |
| Lung ca. NCI-H23 | 3.3 | Brain (fetal) | 0.9 |
| Lung ca. NCI-H460 | 3.8 | Brain (Hippocampus) | 16.3 |
| | | Pool | |
| Lung ca. HOP-62 | 0.7 | Cerebral Cortex Pool | 0.5 |
| Lung ca. NCI-H522 | 1.5 | Brain (Substantia | 4.6 |
| | | nigra) Pool | |
| Liver | 1.2 | Brain (Thalamus) Pool | 9.0 |
| Fetal Liver | 17.2 | Brain (whole) | 1.4 |
| Liver ca. HepG2 | 1.2 | Spinal Cord Pool | 11.6 |
| Kidney Pool | 5.1 | Adrenal Gland | 0.3 |
| Fetal Kidney | 7.1 | Pituitary gland Pool | 3.9 |
| Renal ca. 786-0 | 1.2 | Salivary Gland | 1.2 |
| Renal ca. A498 | 2.4 | Thyroid (female) | 5.5 |
| Renal ca. ACHN | 1.2 | Pancreatic ca. | 0.6 |
| | | CAPAN2 | |
| Renal ca. UO-31 | 2.5 | Pancreas Pool | 8.8 |

CNS_neurodegeneration_v1.0 Summary: Ag3504 Expression of the CG59662-01 gene is low/undetectable (CTs>35) across all of the samples on this panel (data not shown).

General_screening_panel_v1.4 Summary: Ag3504 The CG59662-01 gene is expressed at low le,vels in the majority of samples on this panel, with highest expression in a melanoma cell line (CT=30). The CG59662-01 gene encodes a protein with homology to cyclophilin, a specific high-affinity binding protein for the immunosuppressant agent cyclosporin A.

Among tissues with metabolic or endocrine function, this gene is expressed at low levels in pancreas, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes. Interestingly, this gene is expressed at higher levels in fetal liver (CT=32.5) than in adult liver (CT=36.4), suggesting that expression of this gene can be used to distinguish fetal and adult liver.

In addition, this gene is expressed at low levels in some regions of the central nervous system, including amygdala, hippocampus, substantia nigra, thalamus, and spinal cord. Therefore, this gene may play a role in central nervous system disorders such as Alzheimer's disease, Parkinson's disease, epilepsy, multiple sclerosis, schizophrenia and depression.

Panel 4D Summary: Ag3504 Significant expression of this gene is detected in a liver cirrhosis sample (CT=34.4). Furthermore, expression of this gene is not detected at significant levels in normal adult liver in Panel 1.4, suggesting that its expression is unique to liver cirrhosis. This gene encodes a putative cyclophilin; therefore, small molecule therapeutics could reduce or inhibit fibrosis that occurs in liver cirrhosis. In addition, expression of this putative cyclophilin could also be used for the diagnosis of liver cirrhosis.

CQ. CG59773-01: Splice Variant Of Myomegalin

Expression of gene CG59773-01 was assessed using the primer-probe set Ag3580, described in Table CQA. Results of the RTQ-PCR runs are shown in Tables CQB, CQC and CQD.

TABLE DDA
Probe Name Ag3591

| | | | Start | SEQ ID |
|---|---|---|---|---|
| Primers | Sequences | Length | Position | NO: |
| Forward | 5′-cactgctccacttttgtcttg-3′ | 21 | 1195 | 728 |
| Probe | TET-5′-cataaaaggacccacacaggagaaaa-3′-TAMRA | 26 | 1216 | 729 |
| Reverse | 5′-cttttccacattctttgcattc-3′ | 22 | 1249 | 730 |

TABLE DGD
Panel 4.1D

| | Rel. Exp. (%) | | Rel. Exp. (%) |
|---|---|---|---|
| | Ag3635, Run | | Ag3635, Run |
| Tissue Name | 169960385 | Tissue Name | 169960385 |
| Secondary Th1 act | 26.1 | HUVEC IL-1beta | 12.5 |
| Secondary Th2 act | 19.1 | HUVEC IFN gamma | 34.6 |
| Secondary Tr1 act | 12.7 | HUVEC TNF alpha + | 3.2 |
| | | IFN gamma | |
| Secondary Th1 rest | 2.0 | HUVEC TNF alpha + | 5.3 |
| | | IL4 | |
| Secondary Th2 rest | 8.2 | HUVEC IL-11 | 29.3 |
| Secondary Tr1 rest | 4.8 | Lung Microvascular EC | 12.7 |
| | | none | |
| Primary Th1 act | 16.0 | Lung Microvascular EC | 3.0 |
| | | TNF alpha + IL-1beta | |
| Primary Th2 act | 17.8 | Microvascular Dermal | 6.0 |
| | | EC none | |
| Primary Tr1 act | 13.1 | Microsvasular Dermal | 4.3 |
| | | EC TNF alpha + IL-1beta | |
| Primary Th1 rest | 3.4 | Bronchial epithelium | 12.5 |
| | | TNF alpha + IL-1beta | |
| Primary Th2 rest | 6.3 | Small airway epithelium | 4.5 |
| | | none | |
| Primary Tr1 rest | 5.4 | Small airway epithelium | 11.0 |
| | | TNF alpha + IL-1beta | |
| CD45RA CD4 | 17.1 | Coronery artery SMC rest | 20.4 |
| lymphocyte act | | | |
| CD45RO CD4 | 44.1 | Coronery artery SMC | 21.8 |
| lymphocyte act | | TNF alpha + IL-1beta | |
| CD8 lymphocyte act | 23.3 | Astrocytes rest | 6.4 |
| Secondary CD8 | 42.9 | Astrocytes TNF alpha + | 9.9 |
| lymphocyte rest | | IL-1beta | |
| Secondary CD8 | 7.4 | KU-812 (Basophil) rest | 0.2 |
| lymphocyte act | | | |
| CD4 lymphocyte none | 11.5 | KU-812 (Basophil) | 0.8 |
| | | PMA/ionomycin | |
| 2ry Th1/Th2/Tr1_anti- | 2.4 | CCD1106 | 11.2 |
| CD95 CH11 | | (Keratinocytes) none | |
| LAK cells rest | 36.3 | CCD1106 | 22.8 |
| | | (Keratinocytes) | |
| | | TNF alpha + IL-1beta | |
| LAK cells IL-2 | 9.1 | Liver cirrhosis | 7.4 |
| LAK cells IL-2 + IL-12 | 22.8 | NCI-H292 none | 14.3 |
| LAK cells IL-2 + IFN | 21.8 | NCI-H292 IL-4 | 20.9 |
| gamma | | | |
| LAK cells IL-2 + IL-18 | 21.2 | NCI-H292 IL-9 | 26.1 |
| LAK cells | 21.0 | NCI-H292 IL-13 | 25.5 |
| PMA/ionomycin | | | |
| NK Cells IL-2 rest | 8.7 | NCI-H292 IFN gamma | 13.2 |
| Two Way MLR 3 day | 24.8 | HPAEC none | 32.5 |
| Two Way MLR 5 day | 18.7 | HPAEC TNF alpha + IL- | 14.3 |
| | | 1beta | |
| Two Way MLR 7 day | 21.6 | Lung fibroblast none | 1.3 |
| PBMC rest | 23.0 | Lung fibroblast TNF | 0.8 |
| | | alpha + IL-1beta | |
| PBMC PWM | 71.2 | Lung fibroblast IL-4 | 1.5 |
| PBMC PHA-L | 46.7 | Lung fibroblast IL-9 | 2.9 |
| Ramos (B cell) none | 1.8 | Lung fibroblast IL-13 | 1.4 |
| Ramos (B cell) | 1.5 | Lung fibroblast IFN | 1.2 |
| ionomycin | | gamma | |
| B lymphocytes PWM | 49.7 | Dermal fibroblast | 33.7 |
| | | CCD1070 rest | |
| B lymphocytes CD40L | 68.8 | Dermal fibroblast | 22.5 |
| and IL-4 | | CCD1070 TNF alpha | |
| EOL-1 dbcAMP | 22.7 | Dermal fibroblast | 10.1 |
| | | CCD1070 IL-1beta | |
| EOL-1 dbcAMP | 40.3 | Dermal fibroblast IFN | 6.1 |
| PMA/ionomycin | | gamma | |
| Dendritic cells none | 54.0 | Dermal fibroblast IL-4 | 10.4 |
| Dendritic cells LPS | 73.7 | Dermal Fibroblasts rest | 9.9 |
| Dendritic cells anti- | 100.0 | Neutrophils TNFa + LPS | 0.8 |
| CD40 | | | |
| Monocytes rest | 66.0 | Neutrophils rest | 2.8 |
| Monocytes LPS | 22.4 | Colon | 6.7 |
| Macrophages rest | 86.5 | Lung | 34.6 |
| Macrophages LPS | 13.4 | Thymus | 51.1 |
| HUVEC none | 8.6 | Kidney | 90.8 |
| HUVEC starved | 11.3 | | |

TABLE CQC
General_screening_panel_v1.4

| | Rel. | | Rel. |
|---|---|---|---|
| | Exp. | | Exp. |
| | (%) | | (%) |
| | Ag3580, | | Ag3580, |
| | Run | | Run |
| Tissue Name | 217423587 | Tissue Name | 217423587 |
| Adipose | 20.7 | Renal ca. TK-10 | 20.7 |
| Melanoma* | 76.8 | Bladder | 15.7 |
| Hs688(A).T | | | |
| Melanoma* | 38.7 | Gastric ca. (liver met.) | 50.0 |
| Hs688(B).T | | NCI-N87 | |
| Melanoma* M14 | 7.6 | Gastric ca. KATO III | 13.1 |
| Melanoma* | 81.2 | Colon ca. SW-948 | 5.8 |
| LOXIMVI | | | |
| Melanoma* SK- | 14.9 | Colon ca. SW480 | 12.9 |
| MEL-5 | | | |
| Squamous cell | 10.2 | Colon ca.* (SW480 | 2.6 |
| carcinoma SCC-4 | | met) SW620 | |
| Testis Pool | 4.5 | Colon ca. HT29 | 4.5 |
| Prostate ca.* (bone | 29.1 | Colon ca. HCT-116 | 15.0 |
| met) PC-3 | | | |
| Prostate Pool | 6.1 | Colon ca. CaCo-2 | 29.7 |
| Placenta | 8.2 | Colon cancer tissue | 8.4 |
| Uterus Pool | 7.7 | Colon ca. SW1116 | 2.5 |
| Ovarian ca. | 24.0 | Colon ca. Colo-205 | 2.9 |
| OVCAR-3 | | | |
| Ovarian ca. | 15.4 | Colon ca. SW-48 | 7.1 |
| SK-OV-3 | | | |
| Ovarian ca. | 17.0 | Colon Pool | 18.8 |
| OVCAR-4 | | | |
| Ovarian ca. | 19.8 | Small Intestine Pool | 18.0 |
| OVCAR-5 | | | |
| Ovarian ca. | 15.1 | Stomach Pool | 7.4 |
| IGROV-1 | | | |
| Ovarian ca. | 6.7 | Bone Marrow Pool | 9.9 |
| OVCAR-8 | | | |
| Ovary | 4.4 | Fetal Heart | 9.0 |
| Breast ca. MCF-7 | 28.9 | Heart Pool | 8.2 |
| Breast ca. MDA- | 39.8 | Lymph Node Pool | 22.8 |
| MB-231 | | | |
| Breast ca. BT 549 | 13.9 | Fetal Skeletal Muscle | 6.8 |
| Breast ca. T47D | 49.0 | Skeletal Muscle Pool | 22.7 |
| Breast ca. MDA-N | 7.6 | Spleen Pool | 12.1 |
| Breast Pool | 9.5 | Thymus Pool | 19.9 |
| Trachea | 15.0 | CNS cancer | 35.1 |
| | | (glio/astro) | |
| | | U87-MG | |
| Lung | 4.0 | CNS cancer | 28.9 |
| | | (glio/astro) | |
| | | U-118-MG | |
| Fetal Lung | 30.6 | CNS cancer | 3.4 |
| | | (neuro; met) | |
| | | SK-N-AS | |
| Lung ca. NCI-N417 | 5.4 | CNS cancer | 13.4 |
| | | (astro) | |
| | | SF-539 | |
| Lung ca. LX-1 | 6.6 | CNS cancer (astro) | 57.4 |
| | | SNB-75 | |
| Lung ca. NCI-H146 | 15.8 | CNS cancer (glio) | 9.0 |
| | | SNB-19 | |
| Lung ca. SHP-77 | 25.9 | CNS cancer (glio) | 49.7 |
| | | SF-295 | |
| Lung ca. A549 | 4.2 | Brain (Amygdala) | 15.9 |
| | | Pool | |
| Lung ca. NCI-H526 | 7.6 | Brain (cerebellum) | 100.0 |
| Lung ca. NCI-H23 | 50.0 | Brain (fetal) | 69.3 |
| Lung ca. NCI-H460 | 4.8 | Brain (Hippocampus) | 16.0 |
| | | Pool | |
| Lung ca. HOP-62 | 24.3 | Cerebral Cortex Pool | 26.1 |
| Lung ca. NCI-H522 | 4.5 | Brain | 22.7 |
| | | (Substantia nigra) | |
| | | Pool | |
| Liver | 4.9 | Brain (Thalamus) Pool | 37.4 |
| Fetal Liver | 12.0 | Brain (whole) | 33.4 |
| Liver ca. HepG2 | 11.1 | Spinal Cord Pool | 34.4 |
| Kidney Pool | 54.0 | Adrenal Gland | 5.2 |
| Fetal Kidney | 40.3 | Pituitary gland Pool | 9.7 |
| Renal ca. 786-0 | 13.9 | Salivary Gland | 3.4 |
| Renal ca. A498 | 15.0 | Thyroid (female) | 5.3 |
| Renal ca. ACHN | 1.6 | Pancreatic ca. | 16.4 |
| | | CAPAN2 | |
| Renal ca. UO-31 | 10.2 | Pancreas Pool | 17.2 |

TABLE CQD
Panel 4.1D

| | Rel. | | Rel. |
|---|---|---|---|
| | Exp. | | Exp. |
| | (%) | | (%) |
| | Ag3580, | | Ag3580, |
| | Run | | Run |
| Tissue Name | 169910382 | Tissue Name | 169910382 |
| Secondary Th1 act | 28.3 | HUVEC IL-1beta | 28.5 |
| Secondary Th2 act | 32.8 | HUVEC IFN gamma | 8.0 |
| Secondary Tr1 act | 34.2 | HUVEC TNF alpha + | 30.1 |
| | | IFN gamma | |
| Secondary Th1 rest | 23.7 | HUVEC TNF alpha + | 27.2 |
| | | IL4 | |
| Secondary Th2 rest | 26.4 | HUVEC IL-11 | 8.8 |
| Secondary Tr1 rest | 34.2 | Lung Microvascular | 42.9 |
| | | EC none | |
| Primary Th1 act | 11.2 | Lung Microvascular | 33.2 |
| | | EC TNFalpha + | |
| | | IL-1beta | |
| Primary Th2 act | 10.8 | Microvascular Dermal | 24.8 |
| | | EC none | |
| Primary Tr1 act | 11.0 | Microvascular Dermal | 20.7 |
| | | EC TNFalpha + | |
| | | IL-1beta | |
| Primary Th1 rest | 32.3 | Bronchial epithelium | 18.6 |
| | | TNFalpha + IL1beta | |
| Primary Th2 rest | 31.2 | Small airway | 8.2 |
| | | epithelium none | |
| Primary Tr1 rest | 24.1 | Small airway | 10.7 |
| | | epithelium | |
| | | TNFalpha + IL-beta | |
| CD45RA CD4 | 50.0 | Coronery artery | 10.7 |
| lymphocyte act | | SMC rest | |
| CD45RO CD4 | 25.5 | Coronery artery | 15.6 |
| lymphocyte act | | SMC TNFalpha + | |
| | | IL-1beta | |
| CD8 lymphocyte act | 18.8 | Astrocytes rest | 14.1 |
| Secondary CD8 | 16.3 | Astrocytes | 13.0 |
| lymphocyte rest | | TNFalpha + | |
| | | IL-1beta | |
| Secondary CD8 | 16.3 | KU-812 (Basophil) | 4.0 |
| lynphocyte act | | rest | |
| CD4 lymphocyte | 22.4 | KU-812 (Basophil) | 7.8 |
| none | | PMA/ionomycin | |
| 2ry Th1/Th2/ | 47.6 | CCD1106 | 24.3 |
| Tr1_anti- | | (Keratinocytes) | |
| CD95 CH11 | | none | |
| LAK cells rest | 24.1 | CCD1106 | 26.4 |
| | | (Keratinocytes) | |
| | | TNFalpha + IL-1beta | |
| LAK cells IL-2 | 21.8 | Liver cirrhosis | 12.0 |
| LAK cells IL-2 + | 23.2 | NCI-H292 none | 9.6 |
| IL-12 | | | |
| LAK cells IL-2 + | 29.1 | NCI-H292 IL-4 | 15.5 |
| IFN gamma | | | |
| LAK cells IL-2 + | 32.5 | NCI-H292 IL-9 | 26.2 |
| IL-18 | | | |
| LAK cells | 35.8 | NCI-H292 IL-13 | 15.3 |
| PMA/ionomycin | | | |
| NK Cells IL-2 rest | 26.1 | NCI-H292 IFN gamma | 14.0 |
| Two Way MLR | 35.8 | HPAEC none | 8.2 |
| 3 day | | | |
| Two Way MLR | 19.5 | HPAEC TNF alpha + | 31.9 |
| 5 day | | IL-1beta | |
| Two Way MLR | 46.0 | Lung fibroblast none | 51.8 |
| 7 day | | | |
| PBMC rest | 15.8 | Lung fibroblast | 44.8 |
| | | TNF alpha + | |
| | | IL-1beta | |
| PBMC PWM | 16.0 | Lung fibroblast IL-4 | 32.8 |
| PBMC PHA-L | 13.3 | Lung fibroblast IL-9 | 51.4 |
| Ramos (B cell) none | 16.8 | Lung fibroblast IL-13 | 29.9 |
| Ramos (B cell) | 15.9 | Lung fibroblast IFN | 27.7 |
| ionomycin | | gamma | |
| B lymphocytes | 14.4 | Dermal fibroblast | 38.4 |
| PMW | | CCD1070 rest | |
| B lymphocytes | 16.3 | Dermal fibroblast | 100.0 |
| CD40L and IL-4 | | CCD1070 TNF alpha | |
| EOL-1 dbcAMP | 5.6 | Dermal fibroblast | 48.3 |
| | | CCD1070 IL-1beta | |
| EOL-1 dbcAMP | 29.3 | Dermal fibroblast IFN | 11.0 |
| PMA/ionomycin | | gamma | |
| Dendritic cells none | 13.6 | Dermal fibroblast IL-4 | 22.7 |
| Dendritic cells LPS | 14.4 | Dermal Fibroblasts | 18.8 |
| | | rest | |
| Dendritic cells anti- | 13.5 | Neutrophils TNFa + | 1.7 |
| CD40 | | LPS | |
| Monocytes rest | 12.3 | Neutrophils rest | 2.6 |
| Monocytes LPS | 62.9 | Colon | 15.8 |
| Macrophages rest | 18.4 | Lung | 15.6 |
| Macrophages LPS | 15.3 | Thymus | 21.8 |
| HUVEC none | 12.6 | Kidney | 46.3 |
| HUVEC starved | 17.0 | | |

CNS_neurodegeneration_v1.0 Summary: Ag3580 Results from two experiments using the same probe/primer set are in excellent agreement. This panel confirms the expression of this gene at high to moderate levels in the brains of an independent group of individuals. This gene is found to be upregulated in the temporal cortex of Alzheimer's disease patients. Therefore, therapeutic modulation of this gene or its protein product may be used to decrease neuronal death and treat Alzheimer's disease.

General_screening_panel_v1.4 Summary: Ag3580 The CG59773-01 gene encodes a splice variant of the myomegalin protein, which is a component of the golgi/centrosome and interacts with a cyclic nucleotide phosphodiesterase (ref. 1). Expression of the CG59773-01 gene is highest in the cerebellum (CT=23.8). In addition, this gene is expressed at high levels in all other regions of the central nervous system examined, including amygdala, hippocampus, substantia nigra, thalamus, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

This gene is also expressed at very high levels in a number of melanoma cell lines. Therefore, therapeutic modulation of the activity of this gene or its protein product may be of benefit in the treatment of melanoma.

### References

1. Verde I, Pahlke G, Salanova M, Zhang G, Wang S, Coletti D, Onuffer J, Jin S L, Conti M. Myomegalin is a novel protein of the golgi/centrosome that interacts with a cyclic nucleotide phosphodiesterase. J Biol Chem Apr. 6, 2001;276(14): 11189-98

Panel 4.1D Summary: Ag3580 This gene is expressed at high to moderate levels in a wide range of cell types of significance in the immune response in health and disease. These cells include T cells, B cells, endothelial cells, macrophages, monocytes, dendritic cells, basophils, eosinophils and peripheral blood mononuclear cells, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, therapeutic modulation of the activity of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CR. CG57460-01: N-Acetyltransferase Camello 2

Expression of gene CG57460-01 was assessed using the primer-probe set Ag3273, described in Table CRA. Results of the RTQ-PCR runs are shown in Tables CRB, CRC and CRD.

TABLE CRA
Probe Name Ag3273

| | | | | SEQ |
|---|---|---|---|---|
| | | | Start | ID |
| Primers | Sequences | Length | Position | NO: |
| Forward | 5′-cgctactactacagccgcaa-3′ | 20 | 205 | 692 |
| Probe | TET-5′-gtgatccgcgcctacctggagtg-3′- | 23 | 226 | 693 |
| | TAMRA | | | |
| Reverse | 5′-gggcggcttcatgtagtact-3′ | 20 | 281 | 694 |

TABLE CRB
CNS_neurodegeneration_v1.0

| | Rel. Exp. (%) | Rel. Exp. (%) | | Rel. Exp. (%) | Rel. Exp. (%) |
|---|---|---|---|---|---|
| Tissue | Ag3273, Run | Ag3273, Run | Tissue | Ag3273, Run | Ag3273, Run |
| Name | 210038591 | 230512515 | Name | 210038591 | 230512515 |
| AD 1 Hippo | 15.7 | 18.6 | Control | 12.2 | 12.7 |
| | | | (Path) 3 | | |
| | | | Temporal | | |
| | | | Ctx | | |
| AD 2 Hippo | 28.9 | 23.2 | Control | 40.3 | 35.4 |
| | | | (Path) 4 | | |
| | | | Temporal | | |
| | | | Ctx | | |
| AD 3 Hippo | 11.0 | 11.0 | AD 1 | 20.9 | 18.8 |
| | | | Occipital | | |
| | | | Ctx | | |
| AD 4 Hippo | 10.2 | 13.8 | AD 2 | 0.0 | 0.0 |
| | | | Occipital | | |
| | | | Ctx | | |
| | | | (Missing) | | |
| AD 5 hippo | 100.0 | 100.0 | AD 3 | 15.2 | 13.1 |
| | | | Occipital | | |
| | | | Ctx | | |
| AD 6 Hippo | 21.8 | 22.4 | AD 4 | 22.1 | 23.2 |
| | | | Occipital | | |
| | | | Ctx | | |
| Control 2 | 27.0 | 27.9 | AD 5 | 18.4 | 18.4 |
| Hippo | | | Occipital | | |
| | | | Ctx | | |
| Control 4 | 22.5 | 21.8 | AD 6 | 46.3 | 48.3 |
| Hippo | | | Occipital | | |
| | | | Ctx | | |
| Control (Path) | 8.4 | 9.0 | Control 1 | 8.0 | 11.2 |
| 3 Hippo | | | Occipital | | |
| | | | Ctx | | |
| AD 1 Temporal | 16.5 | 15.1 | Control 2 | 80.1 | 82.4 |
| Ctx | | | Occipital | | |
| | | | Ctx | | |
| AD 2 Temporal | 29.3 | 26.2 | Control 3 | 26.2 | 27.2 |
| Ctx | | | Occipital | | |
| | | | Ctx | | |
| AD 3 Temporal | 10.9 | 12.9 | Control 4 | 14.8 | 14.1 |
| Ctx | | | Occipital | | |
| | | | Ctx | | |
| AD 4 Temporal | 22.5 | 20.3 | Control | 70.2 | 68.8 |
| Ctx | | | (Path) 1 | | |
| | | | Occipital | | |
| | | | Ctx | | |
| AD 5 Inf | 57.0 | 59.5 | Control | 21.6 | 20.6 |
| Temporal Ctx | | | (Path) 2 | | |
| | | | Occipital | | |
| | | | Ctx | | |
| AD 5 | 30.6 | 26.8 | Control | 8.8 | 7.4 |
| Sup Temporal | | | (Path) 3 | | |
| Ctx | | | Occipital | | |
| | | | Ctx | | |
| AD 6 Inf | 27.2 | 30.1 | Control | 36.1 | 34.2 |
| Temporal Ctx | | | (Path) 4 | | |
| | | | Occipital | | |
| | | | Ctx | | |
| AD 6 Sup | 34.2 | 37.4 | Control 1 | 14.1 | 13.7 |
| Temporal Ctx | | | Parietal Ctx | | |
| Control 1 | 12.2 | 11.1 | Control 2 | 33.9 | 36.9 |
| Temporal Ctx | | | Parietal Ctx | | |
| Control 2 | 43.2 | 39.8 | Control 3 | 33.0 | 28.7 |
| Temporal Ctx | | | Parietal Ctx | | |
| Control 3 | 16.7 | 18.3 | Control | 63.7 | 67.8 |
| Temporal Ctx | | | (Path) 1 | | |
| | | | Parietal Ctx | | |
| Control 4 | 18.3 | 19.8 | Control | 30.6 | 30.4 |
| Temporal Ctx | | | (Path) 2 | | |
| | | | Parietal Ctx | | |
| Control (Path) | 51.4 | 48.0 | Control | 8.1 | 9.7 |
| 1 Temporal Ctx | | | (Path) 3 | | |
| | | | Parietal Ctx | | |
| Control (Path) | 39.2 | 45.7 | Control | 64.2 | 59.5 |
| 2 Temporal Ctx | | | (Path) 4 | | |
| | | | Parietal Ctx | | |

TABLE CRC
General_screening_panel_v1.4

| | Rel. | | Rel. |
|---|---|---|---|
| | Exp. | | Exp. |
| | (%) | | (%) |
| | Ag3273, | | Ag3273, |
| | Run | | Run |
| Tissue Name | 215775405 | Tissue Name | 215775405 |
| Adipose | 7.1 | Renal ca. TK-10 | 4.9 |
| Melanoma* | 0.0 | Bladder | 0.9 |
| Hs688(A).T | | | |
| Melanoma* | 0.0 | Gastric ca. (liver met.) | 2.4 |
| Hs688(B).T | | NCI-N87 | |
| Melanoma* M14 | 17.6 | Gastric ca. KATO III | 0.0 |
| Melanoma* | 0.9 | Colon ca. SW-948 | 0.0 |
| LOXIMVI | | | |
| Melanoma* SK- | 4.2 | Colon ca. SW480 | 2.7 |
| MEL-5 | | | |
| Squamous cell | 0.2 | Colon ca.* (SW480 | 1.0 |
| carcinoma SCC-4 | | met) SW620 | |
| Testis Pool | 13.5 | Colon ca. HT29 | 0.1 |
| Prostate ca.* (bone | 2.7 | Colon ca. HCT-116 | 6.0 |
| met) PC-3 | | | |
| Prostate Pool | 0.2 | Colon ca. CaCo-2 | 2.0 |
| Placenta | 0.0 | Colon cancer tissue | 0.6 |
| Uterus Pool | 0.0 | Colon ca. SW1116 | 0.4 |
| Ovarian ca. | 5.4 | Colon ca. Colo-205 | 0.0 |
| OVCAR-3 | | | |
| Ovarian ca. | 6.8 | Colon ca. SW-48 | 0.0 |
| SK-OV-3 | | | |
| Ovarian ca. | 1.6 | Colon Pool | 0.4 |
| OVCAR-4 | | | |
| Ovarian ca. | 2.1 | Small Intestine Pool | 0.1 |
| OVCAR-5 | | | |
| Ovarian ca. | 7.7 | Stomach Pool | 0.3 |
| IGROV-1 | | | |
| Ovarian ca. | 9.6 | Bone Marrow Pool | 0.1 |
| OVCAR-8 | | | |
| Ovary | 0.8 | Fetal Heart | 100.0 |
| Breast ca. MCF-7 | 1.7 | Heart Pool | 0.2 |
| Breast ca. MDA- | 0.8 | Lymph Node Pool | 0.0 |
| MB-231 | | | |
| Breast ca. BT 549 | 7.7 | Fetal Skeletal Muscle | 0.3 |
| Breast ca. T47D | 9.3 | Skeletal Muscle Pool | 1.3 |
| Breast ca. MDA-N | 0.0 | Spleen Pool | 0.0 |
| Breast Pool | 0.4 | Thymus Pool | 0.4 |
| Trachea | 0.2 | CNS cancer | 0.0 |
| | | (glio/astro) | |
| | | U87-MG | |
| Lung | 0.0 | CNS cancer | 0.0 |
| | | (glio/astro) | |
| | | U-118-MG | |
| Fetal Lung | 0.3 | CNS cancer | 0.0 |
| | | (neuro; met) | |
| | | SK-N-AS | |
| Lung ca. NCI-N417 | 5.1 | CNS cancer | 0.2 |
| | | (astro) | |
| | | SF-539 | |
| Lung ca. LX-1 | 0.4 | CNS cancer (astro) | 0.6 |
| | | SNB-75 | |
| Lung ca. NCI-H146 | 12.2 | CNS cancer (glio) | 7.1 |
| | | SNB-19 | |
| Lung ca. SHP-77 | 2.4 | CNS cancer (glio) | 1.8 |
| | | SF-295 | |
| Lung ca. A549 | 3.4 | Brain (Amygdala) | 31.4 |
| | | Pool | |
| Lung ca. NCI-H526 | 12.0 | Brain (cerebellum) | 32.3 |
| Lung ca. NCI-H23 | 5.8 | Brain (fetal) | 9.3 |
| Lung ca. NCI-H460 | 2.3 | Brain (Hippocampus) | 22.5 |
| | | Pool | |
| Lung ca. HOP-62 | 1.0 | Cerebral Cortex Pool | 40.3 |
| Lung ca. NCI-H522 | 7.4 | Brain | 57.4 |
| | | (Substantia nigra) | |
| | | Pool | |
| Liver | 0.0 | Brain (Thalamus) Pool | 36.6 |
| Fetal Liver | 0.1 | Brain (whole) | 24.8 |
| Liver ca. HepG2 | 0.3 | Spinal Cord Pool | 20.2 |
| Kidney Pool | 0.2 | Adrenal Gland | 0.0 |
| Fetal Kidney | 0.7 | Pituitary gland Pool | 2.2 |
| Renal ca. 786-0 | 0.2 | Salivary Gland | 0.2 |
| Renal ca. A498 | 1.0 | Thyroid (female) | 1.1 |
| Renal ca. ACHN | 4.5 | Pancreatic ca. | 0.0 |
| | | CAPAN2 | |
| Renal ca. UO-31 | 3.7 | Pancreas Pool | 0.6 |

TABLE CRD
Panel 4D

| | Rel. Exp. (%) | | Rel. Exp. (%) |
|---|---|---|---|
| | Ag3273, Run | | Ag3273, Run |
| Tissue Name | 165338992 | Tissue Name | 165338992 |
| Secondary Th1 act | 1.0 | HUVEC IL-1beta | 0.0 |
| Secondary Th2 act | 2.5 | HUVEC IFN gamma | 0.0 |
| Secondary Tr1 act | 3.7 | HUVEC TNF alpha+ | 0.0 |
| | | IFN gamma | |
| Secondary Th1 rest | 5.0 | HUVEC TNF alpha+ | 0.0 |
| | | IL4 | |
| Secondary Th2 rest | 0.9 | HUVEC IL-11 | 0.0 |
| Secondary Th1 rest | 6.9 | Lung Microvascular EC | 0.0 |
| | | none | |
| Primary Th1 act | 6.2 | Lung Microvascular EC | 0.0 |
| | | TNFalpha + IL-1beta | |
| Primary Th2 act | 5.9 | Microvascular Dermal | 0.0 |
| | | EC none | |
| Primary Tr1 act | 9.5 | Microsvasular Dermal | 0.0 |
| | | EC TNFalpha + IL-1beta | |
| Primary Th1 rest | 3.1 | Bronchial epithelium | 0.0 |
| | | TNFalpha + IL-1beta | |
| Primary Th2 rest | 0.0 | Small airway epithelium | 2.9 |
| | | none | |
| Primary Tr1 rest | 0.0 | Small airway epithelium | 0.0 |
| | | TNFalpha + IL-1beta | |
| CD45RA CD4 | 0.0 | Coronery artery SMC rest | 1.8 |
| lymphocyte act | | | |
| CD45RO CD4 | 0.0 | Coronery artery SMC | 0.0 |
| lymphocyte act | | TNFalpha + IL-1beta | |
| CD8 lymphocyte act | 0.0 | Astrocytes rest | 16.5 |
| Secondary CD8 | 0.0 | Astrocytes TNFalpha + | 10.4 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 19.1 | CCD1106 | 0.0 |
| CD95 CH11 | | (Keratinocytes) none | |
| | | CCD1106 | |
| LAK cells rest | 0.0 | (Keratinocytes) | 0.0 |
| | | TNFalpha + IL-1beta | |
| LAK cells IL-2 | 1.2 | liver cirrhosis | 12.7 |
| LAK cells IL-2 + IL-12 | 2.5 | Lupus kidney | 3.9 |
| LAK cells IL-2 + IFN | 4.1 | NCI-H292 none | 10.8 |
| gamma | | | |
| LAK cells IL-2 + IL-18 | 0.0 | NCI-H292 IL-4 | 23.2 |
| LAK cells | 0.0 | NCI-H292 IL-9 | 22.4 |
| PMA/ionomycin | | | |
| NK Cells IL-2 rest | 0.0 | NCI-H292 IL-13 | 10.3 |
| Two Way MLR 3 day | 1.1 | NCI-H292 IFN gamma | 18.0 |
| Two Way MLR 5 day | 0.0 | AHPAEC none | 0.0 |
| Two Way MLR 7 day | 0.0 | HPAEC TNF alpha + IL- | 0.0 |
| | | 1 beta | |
| PBMC rest | 0.0 | Lung fibroblast none | 1.9 |
| PBMC PWM | 1.2 | Lung fibroblast TNF | 0.0 |
| | | alpha + IL-1 beta | |
| PBMC PHA-L | 0.0 | Lung fibroblast IL-4 | 3.8 |
| Ramos (B cell) none A | 0.0 | Lung fibroblast IL-9 | 0.5 |
| Ramos (B cell) | 0.0 | Lung fibroblast IL-13 | 5.7 |
| ionomycin | | | |
| B lymphocytes PWM | 3.5 | Lung fibroblast IFN | 2.9 |
| | | gamma | |
| B lymphocytes CD40L | 0.0 | Dermal fibroblast | 0.0 |
| and IL-4 | | CCD1070 rest | |
| EOL-1 dbcAMP | 100.0 | Dermal fibroblast | 0.0 |
| | | CCD1070 TNF alpha | 0.0 |
| EOL-1 dbcAMP | 25.9 | Dermal fibroblast | |
| PMA/ionomycin | | CCD1070 IL-1 beta | |
| Dendritic cells none | 3.8 | Dermal fibroblast IFN | 2.8 |
| | | gamma | |
| Dendritic cells LPS | 2.6 | Dermal fibroblast IL-4 | 4.8 |
| Dendritic cells anti- | 5.0 | IBD Colitis 2 | 0.0 |
| CD40 | | | |
| Monocytes rest | 0.0 | IBD Crohn's | 0.0 |
| Monocytes LPS | 0.0 | Colon | 37.4 |
| Macrophages rest | 4.2 | Lung | 8.4 |
| Macrophages LPS | 0.0 | Thymus | 40.9 |
| HUVEC none | 0.0 | Kidney | 6.3 |
| HUVEC starved | 0.0 | | |

CNS_neurodegeneration_v1.0 Summary: Ag3273 Two experiments with the same probe and primer set produce results that are in excellent agreement. This panel confirms the expression of this gene at low to moderate levels in the brains of an independent group of individuals. Expression of this gene is found to be down-regulated in the temporal cortex of Alzheimer's disease patients. Therefore, up-regulation of this gene or its protein product, or treatment with specific agonists for this protein, may be of use in reversing the dementia/memory loss associated with Alzheimer's disease and neuronal death.

General_screening_panel_v1.4 Summary: Ag3273 Highest expression of the CG57460-01 gene is seen in fetal heart (CT=28.6). In addition, this gene is expressed at much higher levels in fetal heart when compared to expression in the adult heart (CT=38). Thus, expression of this gene may be used to differentiate between the fetal and adult source of this tissue. In addition, the higher expression in fetal heart suggests that this protein product may be involved in the development of this organ. Therefore, therapeutic modulation of the expression or function of this gene may be useful in the treatment of heart disease.

This gene also shows highly specific brain expression. Please see Panel CNS_neurodegeneration for discussion of utility of this gene in the central nervous system.

In addition, expression of this gene appears to be upregulated in a number of cancer cell lines when compared to the normal tissues. Specifically, expression of this gene appears to be higher in ovarian, breast, lung and renal cancer cell lines when compared to their respective normal tissues. Therefore, therapeutic modulation of the activity of this gene or its protein may be of benefit in the treatment of ovarian, breast, lung and renal cancer. The CG57460-01 gene encodes a transmembrane protein with homology to N-acetyltransferase Camello 2, a protein involved in cellular adhesion (ref. 1).

### REFERENCES

1. Popsueva A E, Luchinskaya N N, Ludwig A V, Zinovjeva O Y, Poteryaev D A, Feigelman M M, Ponomarev M B, Berekelya L, Belyavsky A V. Overexpression of camello, a member of a novel protein family, reduces blastomere adhesion and inhibits gastrulation in Xenopus laevis. Dev Biol Jun. 15, 2001;234(2):483-96

Panel 4.1D Summary: Ag3273 Highest expression of the CG57460-01 is seen in eosinophils. In addition, differential expression is observed in the eosinophil cell line EOL-1 under resting conditions over that in EOL-1 cells stimulated by phorbol ester and ionomycin. Thus, this gene may be involved in eosinophil function. Therefore, therapeutic modulation of the expression or function of this gene may reduce eosinophil activation and be useful in the treatment of asthma and allergies.

In addition, significant expression in normal colon and thymus suggest a role for this gene in the normal homeostasis of these tissues. Therefore, therapeutic modulation of the expression or function of this gene may modulate immune function (T cell development) and be important for organ transplant, AIDS treatment or post chemotherapy immune reconstitution. Furthermore, since expression of this gene is decreased in colon samples from patients with IBD colitis and Crohn's disease relative to normal colon, therapeutic modulation of the activity of the protein encoded by this gene may be useful in the treatment of inflammatory bowel disease.

CS. CG57464-01

Expression of gene CG57464-01 was assessed using the primer-probe set Ag3248, described in Table CSA. Results of the RTQ-PCR runs are shown in Tables CSB, CSC, CSD and CSE.

TABLE CSA
Probe Name Ag3248

| | | | | SEQ |
|---|---|---|---|---|
| | | | Start | ID |
| Primers | Sequences | Length | Position | NO: |
| Forward | 5′-cctccctggtagaggtcaac-3′ | 20 | 929 | 695 |
| Probe | TET-5′-ctactcagtgcccagcagccaggt-3′- | 24 | 954 | 696 |
| | TAMRA | | | |
| Reverse | 5′-tgtctgcatgcagcctatg-3′ | 19 | 996 | 697 |

TABLE CSB
CNS_neurodegeneration_v1.0

| | Rel. Exp. (%) | Rel. Exp. (%) | | Rel. Exp. (%) | Rel. Exp. (%) |
|---|---|---|---|---|---|
| Tissue | Ag3248, Run | Ag3248, Run | Tissue | Ag3248, Run | Ag3248, Run |
| Name | 210037962 | 224063124 | Name | 210037962 | 224063124 |
| AD 1 Hippo | 26.4 | 4.8 | Control | 28.5 | 39.2 |
| | | | (Path) 3 | | |
| | | | Temporal | | |
| | | | Ctx | | |
| AD 2 Hippo | 73.2 | 71.7 | Control | 57.8 | 61.1 |
| | | | (Path) 4 | | |
| | | | Temporal | | |
| | | | Ctx | | |
| AD 3 Hippo | 21.6 | 5.6 | AD 1 | 19.1 | 21.2 |
| | | | Occipital | | |
| | | | Ctx | | |
| AD 4 Hippo | 28.3 | 28.9 | AD 2 | 0.0 | 0.0 |
| | | | Occipital | | |
| | | | Ctx | | |
| | | | (Missing) | | |
| AD 5 hippo | 85.3 | 100.0 | AD 3 | 13.0 | 15.9 |
| | | | Occipital | | |
| | | | Ctx | | |
| AD 6 Hippo | 70.2 | 72.7 | AD 4 | 37.4 | 28.7 |
| | | | Occipital | | |
| | | | Ctx | | |
| Control 2 | 64.6 | 49.3 | AD 5 | 33.9 | 85.3 |
| Hippo | | | Occipital | | |
| | | | Ctx | | |
| Control 4 | 55.5 | 64.2 | AD 6 | 58.6 | 14.4 |
| Hippo | | | Occipital | | |
| | | | Ctx | | |
| Control (Path) | 46.3 | 0.9 | Control 1 | 17.3 | 22.7 |
| 3 Hippo | | | Occipital | | |
| | | | Ctx | | |
| AD 1 Temporal | 28.9 | 24.1 | Control 2 | 89.5 | 81.2 |
| Ctx | | | Occipital | | |
| | | | Ctx | | |
| AD 2 Temporal | 57.4 | 62.4 | Control 3 | 68.3 | 62.0 |
| Ctx | | | Occipital | | |
| | | | Ctx | | |
| AD 3 Temporal | 18.9 | 16.7 | Control 4 | 29.3 | 34.2 |
| Ctx | | | Occipital | | |
| | | | Ctx | | |
| AD 4 Temporal | 42.3 | 29.9 | Control | 100.0 | 92.7 |
| Ctx | | | (Path) 1 | | |
| | | | Occipital | | |
| | | | Ctx | | |
| AD 5 Inf | 77.4 | 97.3 | Control | 36.3 | 15.1 |
| Temporal Ctx | | | (Path) 2 | | |
| | | | Occipital | | |
| | | | Ctx | | |
| AD 5 | 69.7 | 87.7 | Control | 32.5 | 25.2 |
| Sup Temporal | | | (Path) 3 | | |
| Ctx | | | Occipital | | |
| | | | Ctx | | |
| AD 6 Inf | 39.2 | 87.1 | Control | 70.2 | 66.9 |
| Temporal Ctx | | | (Path) 4 | | |
| | | | Occipital | | |
| | | | Ctx | | |
| AD 6 Sup | 73.2 | 70.7 | Control 1 | 24.8 | 32.3 |
| Temporal Ctx | | | Parietal Ctx | | |
| Control 1 | 25.3 | 26.1 | Control 2 | 70.7 | 94.0 |
| Temporal Ctx | | | Parietal Ctx | | |
| Control 2 | 43.5 | 77.4 | Control 3 | 59.0 | 0.0 |
| Temporal Ctx | | | Parietal Ctx | | |
| Control 3 | 74.2 | 49.3 | Control | 42.6 | 80.7 |
| Temporal Ctx | | | (Path) 1 | | |
| | | | Parietal Ctx | | |
| Control 4 | 45.7 | 68.8 | Control | 67.4 | 59.9 |
| Temporal Ctx | | | (Path) 2 | | |
| | | | Parietal Ctx | | |
| Control (Path) | 63.3 | 58.6 | Control | 25.3 | 24.1 |
| 1 Temporal Ctx | | | (Path) 3 | | |
| | | | Parietal Ctx | | |
| Control (Path) | 55.9 | 55.1 | Control | 78.5 | 79.0 |
| 2 Temporal Ctx | | | (Path) 4 | | |
| | | | Parietal Ctx | | |

TABLE CSC
General_screening_panel_v1.4

| | Rel. Exp. | | Rel. Exp. |
|---|---|---|---|
| | (%) Ag3248, | | (%) Ag3248, |
| | Run | | Run |
| Tissue Name | 214693634 | Tissue Name | 214693634 |
| Adipose | 1.5 | Renal ca. TK-10 | 3.6 |
| Melanoma* | 6.2 | Bladder | 7.6 |
| Hs688(A).T | | | |
| Melanoma* | 5.8 | Gastric ca. (liver met.) | 16.8 |
| Hs688(B).T | | NCI-N87 | |
| Melanoma* M14 | 4.9 | Gastric ca. KATO III | 14.5 |
| Melanoma* | 5.3 | Colon ca. SW-948 | 6.4 |
| LOXIMVI | | | |
| Melanoma* SK- | 4.3 | Colon ca. SW480 | 15.6 |
| MEL-5 | | | |
| Squamous cell | 3.3 | Colon ca.* (SW480 | 9.1 |
| carcinoma SCC-4 | | met) SW620 | |
| Testis Pool | 1.5 | Colon ca. HT29 | 7.1 |
| Prostate ca.* (bone | 2.3 | Colon ca. HCT-116 | 7.5 |
| met) PC-3 | | | |
| Prostate Pool | 3.1 | Colon ca. CaCo-2 | 8.7 |
| Placenta | 3.0 | Colon cancer tissue | 4.9 |
| Uterus Pool | 0.9 | Colon ca. SW1116 | 10.2 |
| Ovarian ca. | 24.0 | Colon ca. Colo-205 | 6.2 |
| OVCAR-3 | | | |
| Ovarian ca. SK- | 18.7 | Colon ca. SW-48 | 7.5 |
| OV-3 | | | |
| Ovarian ca. | 4.1 | Colon Pool | 4.2 |
| OVCAR-4 | | | |
| Ovarian ca. | 33.0 | Small Intestine Pool | 4.5 |
| OVCAR-5 | | | |
| Ovarian ca. | 16.0 | Stomach Pool | 2.7 |
| IGROV-1 | | | |
| Ovarian ca. | 20.9 | Bone Marrow Pool | 1.3 |
| OVCAR-8 | | | |
| Ovary | 3.1 | Fetal Heart | 3.2 |
| Breast ca. MCF-7 | 4.5 | Heart Pool | 2.6 |
| Breast ca. MDA- | 12.2 | Lymph Node Pool | 4.6 |
| MB-231 | | | |
| Breast ca. BT 549 | 12.0 | Fetal Skeletal Muscle | 1.2 |
| Breast ca. T47D | 100.0 | Skeletal Muscle Pool | 4.0 |
| Breast ca. MDA-N | 9.7 | Spleen Pool | 4.1 |
| Breast Pool | 5.2 | Thymus Pool | 5.3 |
| Trachea | 3.1 | CNS cancer | 12.1 |
| | | (glio/astro) U87-MG | |
| Lung | 0.4 | CNS cancer | 10.9 |
| | | (glio/astro) U-118-MG | |
| Fetal Lung | 4.4 | CNS cancer | 16.0 |
| | | (neuro; met) SK-N-AS | |
| Lung ca. NCI-N417 | 3.1 | CNS cancer (astro) | 6.2 |
| | | SF-539 | |
| Lung ca. LX-1 | 12.5 | CNS cancer (astro) | 15.3 |
| | | SNB-75 | |
| Lung ca. NCI-H146 | 5.1 | CNS cancer (glio) | 13.2 |
| | | SNB-19 | |
| Lung ca. SHP-77 | 1.5 | CNS cancer (glio) SF- | 25.9 |
| | | 295 | |
| Lung ca. A549 | 5.4 | Brain (Amygdala) | 4.7 |
| | | Pool | |
| Lung ca. NCI-H526 | 7.4 | Brain (cerebellum) | 8.5 |
| Lung ca. NCI-H23 | 4.4 | Brain (fetal) | 2.1 |
| Lung ca. NCI-H460 | 4.5 | Brain (Hippocampus) | 6.1 |
| | | Pool | |
| Lung ca. HOP-62 | 6.3 | Cerebral Cortex Pool | 6.0 |
| Lung ca. NCI-H522 | 5.5 | Brain (Substantia | 8.1 |
| | | nigra) Pool | |
| Liver | 3.2 | Brain (Thalamus) Pool | 7.8 |
| Fetal Liver | 2.3 | Brain (whole) | 3.4 |
| Liver ca. HepG2 | 5.4 | Spinal Cord Pool | 6.6 |
| Kidney Pool | 4.5 | Adrenal Gland | 3.0 |
| Fetal Kidney | 3.1 | Pituitary gland Pool | 3.4 |
| Renal ca. 786-0 | 7.4 | Salivary Gland | 4.3 |
| Renal ca. A498 | 5.8 | Thyroid (female) | 6.1 |
| Renal ca. ACHN | 5.6 | Pancreatic ca. | 11.3 |
| | | CAPAN2 | |
| Renal ca. UO-31 | 6.1 | Pancreas Pool | 5.2 |

TABLE CSD
Panel 2.2

| | Rel. Exp. (%) | | Rel. Exp. (%) |
|---|---|---|---|
| | Ag3248, Run | | Ag3248, |
| Tissue Name | 174441298 | Tissue Name | Run 174441298 |
| Normal Colon | 14.4 | Kidney Margin | 100.0 |
| | | (OD04348) | |
| Colon cancer | 6.3 | Kidney malignant | 6.3 |
| (OD06064) | | cancer (OD06204B) | |
| Colon Margin | 7.5 | Kidney normal | 9.3 |
| (OD06064) | | adjacent tissue | |
| | | (OD06204E) | |
| Colon cancer | 8.0 | Kidney Cancer | 57.8 |
| (OD06159) | | (OD04450-01) | |
| Colon Margin | 5.3 | Kidney Margin | 16.2 |
| (OD06159) | | (OD04450-03) | |
| Colon cancer | 2.4 | Kidney Cancer | 10.8 |
| (OD06297-04) | | 8120613 | |
| Colon Margin | 6.3 | Kidney Margin | 16.7 |
| (OD06297-05) | | 8120614 | |
| CC Gr.2 ascend colon | 14.1 | Kidney Cancer | 9.0 |
| (ODO3921) | | 9010320 | |
| CC Margin (ODO3921) | 12.8 | Kidney Margin | 19.5 |
| | | 9010321 | |
| Colon cancer metastasis | 4.4 | Kidney Cancer | 34.2 |
| (OD06104) | | 8120607 | |
| Lung Margin | 4.8 | Kidney Margin | 27.7 |
| (OD06104) | | 8120608 | |
| Colon mets to lung | 15.0 | Normal Uterus | 3.8 |
| (OD04451-01) | | | |
| Lung Margin | 6.7 | Uterine Cancer 064011 | 10.5 |
| (OD04451-02) | | | |
| Normal Prostate | 9.0 | Normal Thyroid | 4.6 |
| Prostate Cancer | 7.5 | Thyroid Cancer | 18.6 |
| (OD04410) | | 064010 | |
| Prostate Margin | 11.6 | Thyroid Cancer | 24.0 |
| (OD04410) | | A302152 | |
| Normal Ovary | 28.7 | Thyroid Margin | 13.3 |
| | | A302153 | |
| Ovarian cancer | 3.1 | Normal Breast | 4.0 |
| (OD06283-03) | | | |
| Ovarian Margin | 1.1 | Breast Cancer | 9.3 |
| (OD06283-07) | | (OD04566) | |
| Ovarian Cancer 064008 | 6.8 | Breast Cancer 1024 | 6.9 |
| Ovarian cancer | 8.6 | Breast Cancer | 86.5 |
| (OD06145) | | (OD04590-01) | |
| Ovarian Margin | 17.8 | Breast Cancer Mets | 22.4 |
| (OD06145) | | (OD04590-03) | |
| Ovarian cancer | 2.0 | Breast Cancer | 47.3 |
| (OD06455-03) | | Metastasis (OD04655- | |
| | | 05) | |
| Ovarian Margin | 2.5 | Breast Cancer 064006 | 9.8 |
| (OD06455-07) | | | |
| Normal Lung | 4.7 | Breast Cancer 9100266 | 5.8 |
| Invasive poor diff. lung | 9.0 | Breast Margin | 3.3 |
| adeno (ODO4945-01) | | 9100265 | |
| Lung Margin | 4.5 | Breast Cancer | 7.3 |
| (ODO4945-03) | | A209073 | |
| Lung Malignant Cancer | 10.6 | Breast Margin | 17.0 |
| (OD03126) | | A2090734 | |
| Lung Margin | 8.4 | Breast cancer | 8.6 |
| (OD03126) | | (OD06083) | |
| Lung Cancer | 8.7 | Breast cancer node | 8.2 |
| (OD05014A) | | metastasis (OD06083) | |
| Lung Margin | 8.6 | Normal Liver | 33.4 |
| (OD05014B) | | | |
| Lung cancer | 6.3 | Liver Cancer 1026 | 20.6 |
| (OD06081) | | | |
| Lung Margin | 4.4 | Liver Cancer 1025 | 29.1 |
| (OD06081) | | | |
| Lung Cancer | 2.7 | Liver Cancer 6004-T | 27.4 |
| (OD04237-01) | | | |
| Lung Margin | 16.7 | Liver Tissue 6004-N | 2.5 |
| (OD04237-02) | | | |
| Ocular Melanoma | 25.0 | Liver Cancer 6005-T | 26.2 |
| Metastasis | | | |
| Ocular Melanoma | 15.8 | Liver Tissue 6005-N | 58.2 |
| Margin (Liver) | | | |
| Melanoma Metastasis | 3.8 | Liver Cancer 064003 | 52.5 |
| Melanoma Margin | 2.6 | Normal Bladder | 10.7 |
| (Lung) | | | |
| Normal Kidney | 12.4 | Bladder Cancer 1023 | 6.8 |
| Kidney Ca, Nuclear | 32.3 | Bladder Cancer | 3.8 |
| grade 2 (OD04338) | | A302173 | |
| Kidney Margin | 7.1 | Normal Stomach | 16.8 |
| (OD04338) | | | |
| Kidney Ca Nuclear | 71.7 | Gastric Cancer | 9.2 |
| grade 1/2 (OD04339) | | 9060397 | |
| Kidney Margin | 14.6 | Stomach Margin | 10.4 |
| (OD04339) | | 9060396 | |
| Kidney Ca, Clear cell | 8.4 | Gastric Cancer | 1.7 |
| type (OD04340) | | 9060395 | |
| Kidney Margin | 31.2 | Stomach Margin | 7.5 |
| (OD04340) | | 9060394 | |
| Kidney Ca, Nuclear | 7.5 | Gastric Cancer 064005 | 10.7 |
| grade 3 (OD04348) | | | |

TABLE CSE
Panel 4D

| | Rel. Exp. (%) | | Rel. Exp. (%) |
|---|---|---|---|
| | Ag3248, Run | | Ag3248, Run |
| Tissue Name | 164390952 | Tissue Name | 164390952 |
| Secondary Th1 act | 15.3 | HUVEC IL-1beta | 1.7 |
| Secondary Th2 act | 16.5 | HUVEC IFN gamma | 16.0 |
| Secondary Tr1 act | 15.5 | HUVEC TNF alpha + | 7.8 |
| | | IFN gamma | |
| Secondary Th1 rest | 8.0 | HUVEC TNF alpha + | 9.2 |
| | | IL4 | |
| Secondary Th2 rest | 8.8 | HUVEC IL-11 | 12.8 |
| Secondary Tr1 rest | 12.8 | Lung Microvascular EC | 19.1 |
| | | none | |
| Primary Th1 act | 8.4 | Lung Microvascular EC | 13.8 |
| | | TNF alpha + IL-1beta | |
| Primary Th2 act | 9.3 | Microvascular Dermal | 18.0 |
| | | EC none | |
| Primary Tr1 act | 15.1 | Microsvasular Dermal | 6.9 |
| | | EC TNF alpha + IL-1beta | |
| Primary Th1 rest | 12.3 | Bronchial epithelium | 21.3 |
| | | TNF alpha + IL1beta | |
| Primary Th2 rest | 4.6 | Small airway epithelium | 13.5 |
| | | none | |
| Primary Tr1 rest | 5.6 | Small airway epithelium | 45.4 |
| | | TNF alpha + IL-1beta | |
| CD45RA CD4 | 12.1 | Coronery artery SMC rest | 12.9 |
| lymphocyte act | | | |
| CD45RO CD4 | 12.0 | Coronery artery SMC | 15.2 |
| lymphocyte act | | TNF alpha + IL-1beta | |
| CD8 lymphocyte act | 12.5 | Astrocytes rest | 18.6 |
| Secondary CD8 | 13.7 | Astrocytes TNF alpha + | 17.2 |
| lymphocyte rest | | IL-1beta | |
| Secondary CD8 | 9.9 | KU-812 (Basophil) rest | 8.7 |
| lymphocyte act | | | |
| CD4 lymphocyte none | 6.8 | KU-812 (Basophil) | 8.6 |
| | | PMA/ionomycin | |
| 2ry Th1/Th2/Tr1_anti- | 9.5 | CCD1106 | 10.9 |
| CD95 CH11 | | (Keratinocytes) none | |
| LAK cells rest | 15.2 | CCD1106 | 12.2 |
| | | (Keratinocytes) | |
| | | TNF alpha + IL-1beta | |
| LAK cells IL-2 | 13.6 | Liver cirrhosis | 7.4 |
| LAK cells IL-2 + IL-12 | 9.1 | Lupus kidney | 10.7 |
| LAK cells IL-2 + IFN | 13.9 | NCI-H292 none | 42.6 |
| gamma | | | |
| LAK cells IL-2 + IL-18 | 9.7 | NCI-H292 IL-4 | 48.3 |
| LAK cells | 1.5 | NCI-H292 IL-9 | 54.3 |
| PMA/ionomycin | | | |
| NK Cells IL-2 rest | 12.9 | NCI-H292 IL-13 | 37.1 |
| Two Way MLR 3 day | 19.8 | NCI-H292 IFN gamma | 48.3 |
| Two Way MLR 5 day | 6.3 | HPAEC none | 17.9 |
| Two Way MLR 7 day | 9.7 | HPAEC TNF alpha + IL- | 5.0 |
| | | 1beta | |
| PBMC rest | 10.7 | Lung fibroblast none | 21.5 |
| PBMC PWM | 27.0 | Lung fibroblast TNF | 27.9 |
| | | alpha + IL-1beta | |
| PBMC PHA-L | 12.0 | Lung fibroblast IL-4 | 31.9 |
| Ramos (B cell) none | 27.9 | Lung fibroblast IL-9 | 35.4 |
| Ramos (B cell) | 100.0 | Lung fibroblast IL-13 | 18.6 |
| ionomycin | | | |
| B lymphocytes PWM | 21.0 | Lung fibroblast IFN | 37.4 |
| | | gamma | |
| B lymphocytes CD40L | 8.9 | Dermal fibroblast | 17.7 |
| and IL-4 | | CCD1070 rest | |
| EOL-1 dbcAMP | 19.8 | Dermal fibroblast | 21.2 |
| | | CCD1070 TNF alpha | |
| EOL-1 dbcAMP | 1.4 | Dermal fibroblast | 8.4 |
| PMA/ionomycin | | CCD1070 IL-1beta | |
| Dendritic cells none | 18.3 | Dermal fibroblast IFN | 16.8 |
| | | gamma | |
| Dendritic cells LPS | 20.0 | Dermal fibroblast IL-4 | 27.7 |
| Dendritic cells anti- | 22.1 | IBD Colitis 2 | 3.0 |
| CD40 | | | |
| Monocytes rest | 12.6 | IBD Crohn's | 4.2 |
| Monocytes LPS | 2.6 | Colon | 28.5 |
| Macrophages rest | 20.6 | Lung | 12.0 |
| Macrophages LPS | 10.4 | Thymus | 53.6 |
| HUVEC none | 11.9 | Kidney | 20.0 |
| HUVEC starved | 15.6 | | |

CNS_neurodegeneration_v1.0 Summary: Ag3248 Results from two experiments using the same probe/primer set gave results that are in excellent agreement. This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3248 Expression of the CG57464-01 gene is highest in a breast cancer cell line (CT=27). This also gene appears to be overexpressed in ovarian and CNS cancer cell lines when compared to the normal tissue controls. Thus, therapeutic modulation of the activity of this gene or its protein may be of benefit in the treatment of breast, ovarian and CNS cancer.

In addition, this gene is expressed at low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 2.2 Summary: Ag3248 This gene is expressed at low to moderate levels in the majority of samples on this panel, with highest expression detected in a sample derived from normal kidney (CT=28.6). Expression of the CG57464-01 gene appears to be upregulated in a number of breast cancer samples when compared to normal breast. Thus, therapeutic modulation of the activity of this gene or its protein product may be of benefit in the treatment of breast cancer.

Panel 4D Summary: Ag3248 Expression of the CG57464-01 gene is highest in Ramos B cells treated with ionomycin (CT=29). Therefore, expression of this gene may be used as a marker of activated B cells. In addition, this gene is expressed at relatively high levels in lung fibroblasts as well as in the mucoepidermoid cell line NCI-H292 independent of treatment (CTs=30), suggesting that therapeutic modulation of the activity of this gene or its protein product may be of benefit in the treatment of asthma and emphysema.

This gene is also expressed at low to moderate levels in a wide range of other cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues.

This pattern is in agreement with the expression profile in General_screening_panel_v1.5 and also suggests a role for the gene product in cell survival and proliferation.

Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CT. CG57466-01: Acetylglucosaminyltransferase

Expression of gene CG57466-01 was assessed using the primer-probe set Ag3249, described in Table CTA. Results of the RTQ-PCR runs are shown in Tables CTB, CTC and CTD.

TABLE CTA
Probe Name Ag3249

| | | | Start | SEQ ID |
|---|---|---|---|---|
| Primers | Sequences | Length | Position | NO: |
| Forward | 5′-accactaactgctcagccaata-3′ | 22 | 156 | 698 |
| Probe | TET-5′-aacttgacccaccagccctggtt-3′-TAMRA | 23 | 180 | 699 |
| Reverse | 5′-tagaagagaaactgccggaact-3′ | 22 | 220 | 700 |

TABLE CTB
CNS_neurodegeneration_v1.0

| | Rel. | | Rel. |
|---|---|---|---|
| | Exp. (%) | | Exp. (%) |
| | Ag3249, | | Ag3249, |
| | Run | | Run |
| Tissue Name | 210037963 | Tissue Name | 210037963 |
| AD 1 Hippo | 8.4 | Control (Path) 3 | 5.6 |
| | | Temporal Ctx | |
| AD 2 Hippo | 41.5 | Control (Path) 4 | 27.2 |
| | | Temporal Ctx | |
| AD 3 Hippo | 14.0 | AD 1 Occipital Ctx | 6.6 |
| AD 4 Hippo | 26.1 | AD 2 Occipital Ctx | 0.0 |
| | | (Missing) | |
| AD 5 Hippo | 29.9 | AD 3 Occipital Ctx | 8.5 |
| AD 6 Hippo | 100.0 | AD 4 Occipital Ctx | 25.2 |
| Control 2 Hippo | 42.0 | AD 5 Occipital Ctx | 48.0 |
| Control 4 Hippo | 29.3 | AD 6 Occipital Ctx | 22.4 |
| Control (Path) 3 | 5.6 | Control 1 Occipital | 6.1 |
| Hippo | | Ctx | |
| AD 1 Temporal | 10.7 | Control 2 Occipital | 51.4 |
| Ctx | | Ctx | |
| AD 2 Temporal | 25.5 | Control 3 Occipital | 22.1 |
| Ctx | | Ctx | |
| AD 3 Temporal | 9.9 | Control 4 Occipital | 15.8 |
| Ctx | | Ctx | |
| AD 4 Temporal | 29.9 | Control (Path) 1 | 92.7 |
| Ctx | | Occipital Ctx | |
| AD 5 Inf Temporal | 25.5 | Control (Path) 2 | 18.7 |
| Ctx | | Occipital Ctx | |
| AD 5 Sup | 27.4 | Control (Path) 3 | 0.7 |
| Temporal Ctx | | Occipital Ctx | |
| AD 6 Inf Temporal | 87.1 | Control (Path) 4 | 29.9 |
| Ctx | | Occipital Ctx | |
| AD 6 Sup | 5.0 | Control 1 Parietal | 13.7 |
| Temporal Ctx | | Ctx | |
| Control 1 | 24.5 | Control 2 Parietal | 29.1 |
| Temporal Ctx | | Ctx | |
| Control 2 | 41.2 | Control 3 Parietal | 25.7 |
| Temporal Ctx | | Ctx | |
| Control 3 | 33.7 | Control (Path) 1 | 64.2 |
| Temporal Ctx | | Parietal Ctx | |
| Control 3 | 21.9 | Control (Path) 2 | 25.9 |
| Temporal Ctx | | Parietal Ctx | |
| Control (Path) 1 | 69.7 | Control (Path) 3 | 1.9 |
| Temporal Ctx | | Parietal Ctx | |
| Control (Path) 2 | 26.4 | Control (Path) 4 | 39.8 |
| Temporal Ctx | | Parietal Ctx | |

TABLE CTC
General_screening_panel_v1.4

| | Rel. | | Rel. |
|---|---|---|---|
| | Exp. (%) | | Exp. (%) |
| | Ag3249, | | Ag3249, |
| | Run | | Run |
| Tissue Name | 214693635 | Tissue Name | 214693635 |
| Adipose | 5.6 | Renal ca. TK-10 | 2.7 |
| Melanoma* | 0.4 | Bladder | 59.5 |
| Hs688(A).T | | | |
| Melanoma* | 0.5 | Gastric ca. (liver met.) | 78.5 |
| Hs688(B).T | | NCI-N87 | |
| Melanoma* M14 | 8.8 | Gastric ca. KATO III | 60.3 |
| Melanoma* | 0.1 | Colon ca. SW-948 | 0.5 |
| LOXIMVI | | | |
| Melanoma* SK- | 15.2 | Colon ca. SW480 | 4.4 |
| MEL-5 | | | |
| Squamous cell | 50.7 | Colon ca.* (SW480 | 0.9 |
| carcinoma SCC-4 | | met) SW620 | |
| Testis Pool | 1.7 | Colon ca. HT29 | 0.8 |
| Prostate ca.* (bone | 9.0 | Colon ca. HCT-116 | 13.2 |
| met) PC-3 | | | |
| Prostate Pool | 1.8 | Colon ca. CaCo-2 | 6.6 |
| Placenta | 10.9 | Colon cancer tissue | 26.6 |
| Uterus Pool | 0.9 | Colon ca. SW1116 | 3.6 |
| Ovarian ca. | 3.8 | Colon ca. Colo-205 | 0.1 |
| OVCAR-3 | | | |
| Ovarian ca. SK- | 6.1 | Colon ca. SW-48 | 0.0 |
| OV-3 | | | |
| Ovarian ca. | 1.3 | Colon Pool | 6.2 |
| OVCAR-4 | | | |
| Ovarian ca. | 22.1 | Small Intestine Pool | 11.3 |
| OVCAR-5 | | | |
| Ovarian ca. | 7.5 | Stomach Pool | 6.7 |
| IGROV-1 | | | |
| Ovarian ca. | 1.4 | Bone Marrow Pool | 2.3 |
| OVCAR-8 | | | |
| Ovary | 24.5 | Fetal Heart | 11.6 |
| Breast ca. MCF-7 | 0.7 | Heart Pool | 2.7 |
| Breast ca. MDA- | 1.4 | Lymph Node Pool | 7.1 |
| MB-231 | | | |
| Breast ca. BT 549 | 1.8 | Fetal Skeletal Muscle | 0.4 |
| Breast ca. T47D | 37.9 | Skeletal Muscle Pool | 0.8 |
| Breast ca. MDA-N | 100.0 | Spleen Pool | 13.3 |
| Breast Pool | 4.0 | Thymus Pool | 3.7 |
| Trachea | 65.5 | CNS cancer | 0.0 |
| | | (glio/astro) U87-MG | |
| Lung | 5.8 | CNS cancer | 0.7 |
| | | (glio/astro) U-118-MG | |
| Fetal Lung | 87.7 | CNS cancer | 21.3 |
| | | (neuro; met) SK-N-AS | |
| Lung ca. NCI-N417 | 0.0 | CNS cancer (astro) | 0.1 |
| | | SF-539 | |
| Lung ca. LX-1 | 29.5 | CNS cancer (astro) | 12.2 |
| | | SNB-75 | |
| Lung ca. NCI-H146 | 0.6 | CNS cancer (glio) | 6.5 |
| | | SNB-19 | |
| Lung ca. SHP-77 | 0.2 | CNS cancer (glio) SF- | 6.2 |
| | | 295 | |
| Lung ca. A549 | 12.9 | Brain (Amygdala) | 5.1 |
| | | Pool | |
| Lung ca. NCI-H526 | 0.0 | Brain (cerebellum) | 6.8 |
| Lung ca. NCI-H23 | 2.3 | Brain (fetal) | 4.5 |
| Lung ca. NCI-H460 | 0.2 | Brain (Hippocampus) | 6.7 |
| | | Pool | |
| Lung ca. HOP-62 | 5.0 | Cerebral Cortex Pool | 6.7 |
| Lung ca. NCI-H522 | 0.4 | Brain (Substantia | 7.6 |
| | | nigra) Pool | |
| Liver | 0.8 | Brain (Thalamus) Pool | 9.0 |
| Fetal Liver | 4.8 | Brain (whole) | 7.5 |
| Liver ca. HepG2 | 0.0 | Spinal Cord Pool | 4.6 |
| Kidney Pool | 11.7 | Adrenal Gland | 40.6 |
| Fetal Kidney | 4.5 | Pituitary gland Pool | 10.5 |
| Renal ca. 786-0 | 2.5 | Salivary Gland | 5.7 |
| Renal ca. A498 | 1.4 | Thyroid (female) | 5.8 |
| Renal ca. ACHN | 22.2 | Pancreatic ca. | 43.8 |
| | | CAPAN2 | |
| Renal ca. UO-31 | 32.8 | Pancreas Pool | 11.3 |

TABLE CTD
Panel 4D

| | Rel. Exp. (%) | | Rel. Exp. (%) |
|---|---|---|---|
| | Ag3249, Run | | Ag3249, Run |
| Tissue Name | 164390953 | Tissue Name | 164390953 |
| Secondary Th1 act | 0.2 | HUVEC IL-1beta | 0.4 |
| Secondary Th2 act | 0.0 | HUVEC IFN gamma | 1.0 |
| Secondary Tr1 act | 0.0 | HUVEC TNF alpha + | 0.5 |
| | | IFN gamma | |
| Secondary Th1 rest | 0.0 | HUVEC TNF alpha + | 0.6 |
| | | IL4 | |
| Secondary Th2 rest | 0.1 | HUVEC IL-11 | 0.9 |
| Secondary Tr1 rest | 0.0 | Lung Microvascular EC | 1.4 |
| | | none | |
| Primary Th1 act | 0.2 | Lung Microvascular EC | 4.5 |
| | | TNF alpha + IL-1beta | |
| Primary Th2 act | 0.0 | Microvascular Dermal | 0.9 |
| | | EC none | |
| Primary Tr1 act | 0.0 | Microsvasular Dermal | 7.7 |
| | | EC TNF alpha + IL-1beta | |
| Primary Th1 rest | 0.1 | Bronchial epithelium | 3.6 |
| | | TNF alpha + IL1beta | |
| Primary Th2 rest | 0.0 | Small airway epithelium | 0.2 |
| | | none | |
| Primary Tr1 rest | 0.1 | Small airway epithelium | 5.8 |
| | | TNF alpha + IL-1beta | |
| CD45RA CD4 | 0.5 | Coronery artery SMC rest | 0.0 |
| lymphocyte act | | | |
| CD45RO CD4 | 0.1 | Coronery artery SMC | 0.0 |
| lymphocyte act | | TNF alpha + IL-1beta | |
| CD8 lymphocyte act | 0.6 | Astrocytes rest | 0.8 |
| Secondary CD8 | 0.0 | Astrocytes TNF alpha + | 3.1 |
| lymphocyte rest | | IL-1beta | |
| Secondary CD8 | 0.1 | KU-812 (Basophil) rest | 2.7 |
| lymphocyte act | | | |
| CD4 lymphocyte none | 1.1 | KU-812 (Basophil) | 3.8 |
| | | PMA/ionomycin | |
| 2ry Th1/Th2/Tr1_anti- | 0.1 | CCD1106 | 1.6 |
| CD95 CH11 | | (Keratinocytes) none | |
| LAK cells rest | 1.9 | CCD1106 | 1.3 |
| | | (Keratinocytes) | |
| | | TNF alpha + IL-1beta | |
| LAK cells IL-2 | 5.0 | Liver cirrhosis | 3.2 |
| LAK cells IL-2 + IL-12 | 1.6 | Lupus kidney | 1.3 |
| LAK cells IL-2 + IFN | 5.0 | NCI-H292 none | 100.0 |
| gamma | | | |
| LAK cells IL-2 + IL-18 | 4.3 | NCI-H292 IL-4 | 87.7 |
| LAK cells | 6.1 | NCI-H292 IL-9 | 96.6 |
| PMA/ionomycin | | | |
| NK Cells IL-2 rest | 12.7 | NCI-H292 IL-13 | 54.0 |
| Two Way MLR 3 day | 0.6 | NCI-H292 IFN gamma | 25.2 |
| Two Way MLR 5 day | 1.6 | HPAEC none | 0.4 |
| Two Way MLR 7 day | 0.9 | HPAEC TNF alpha + IL- | 6.3 |
| | | 1beta | |
| PBMC rest | 9.4 | Lung fibroblast none | 0.1 |
| PBMC PWM | 3.7 | Lung fibroblast TNF | 0.3 |
| | | alpha + IL-1beta | |
| PBMC PHA-L | 0.7 | Lung fibroblast IL-4 | 0.7 |
| Ramos (B cell) none | 0.1 | Lung fibroblast IL-9 | 0.0 |
| Ramos (B cell) | 2.0 | Lung fibroblast IL-13 | 0.3 |
| ionomycin | | | |
| B lymphocytes PWM | 2.7 | Lung fibroblast IFN | 0.4 |
| | | gamma | |
| B lymphocytes CD40L | 5.8 | Dermal fibroblast | 0.1 |
| and IL-4 | | CCD1070 rest | |
| EOL-1 dbcAMP | 26.6 | Dermal fibroblast | 0.8 |
| | | CCD1070 TNF alpha | |
| EOL-1 dbcAMP | 8.4 | Dermal fibroblast | 0.0 |
| PMA/ionomycin | | CCD1070 IL-1beta | |
| Dendritic cells none | 3.2 | Dermal fibroblast IFN | 0.0 |
| | | gamma | |
| Dendritic cells LPS | 2.5 | Dermal fibroblast IL-4 | 0.0 |
| Dendritic cells anti- | 1.3 | IBD Colitis 2 | 1.4 |
| CD40 | | | |
| Monocytes rest | 0.4 | IBD Crohn's | 4.2 |
| Monocytes LPS | 10.5 | Colon | 48.6 |
| Macrophages rest | 1.1 | Lung | 19.5 |
| Macrophages LPS | 17.9 | Thymus | 2.5 |
| HUVEC none | 0.8 | Kidney | 3.9 |
| HUVEC starved | 1.1 | | |

CNS_neurodegeneration_v1.0 Summary: Ag3249 This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3249 The CG57466-01 gene encodes a protein with homology to beta-1,3-galactosyltransferases, which catalyze the formation of type I oligosaccharides (ref. 1). Expression of this gene is highest in a breast cancer cell line (CT=28.1). In addition, expression of this gene appears to be upregulated in pancreatic and gastric cancer cell lines when compared to their respective normal tissues. Thus, therapeutic modulation of the activity of this gene or its protein product may be of benefit in the treatment of breast, pancreatic and gastric cancer.

This gene also shows significant levels of expression in trachea, bladder and fetal lung. Interestingly, CG57466-01 gene expression is much higher in fetal lung (CT=28.3) than in adult lung (CT=32.2), suggesting that expression of this gene can be used to distinguish adult from fetal lung.

In addition, this gene is expressed at low levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Among tissues with metabolic or endocrine function, this gene is expressed at low to moderate levels in pancreas, adipose, adrenal gland, thyroid, pituitary gland, heart, fetal liver and the gastrointestinal tract. Therefore, therapeutic modulation of the activity of this gene may prove useful in the treatment of endocrine/metabolically related diseases, such as obesity and diabetes.

### REFERENCES

1. Shiraishi N, Natsume A, Togayachi A, Endo T, Akashima T, Yamada Y, Imai N, Nakagawa S, Koizumi S, Sekine S, Narimatsu H, Sasaki K. Identification and characterization of three novel beta 1,3-N-acetylglucosaminyltransferases structurally related to the beta 1,3-galactosyltransferase family. J Biol Chem Feb. 2, 2001;276(5):3498

Panel 4D Summary: Ag3249 This transcript is most highly expressed in a cluster of treated and untreated samples derived from the NCI-H292 cell line, a human airway epithelial cell line that produces mucins (CTs=30-32). Mucus overproduction is an important feature of bronchial asthma and chronic obstructive pulmonary disease samples. The transcript is also expressed at lower but still significant levels in small airway epithelium treated with IL-1 beta and TNF-alpha. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

CU. CG57468-01: Multidrug Resistance Protein 1

Expression of gene CG57468-01 was assessed using the primer-probe set Ag3250, described in Table CUA. Results of the RTQ-PCR runs are shown in Tables CUB.

TABLE CUA
Probe Name Ag3250

| | | | Start | SEQ ID |
|---|---|---|---|---|
| Primers | Sequences | Length | Position | NO: |
| Forward | 5′-agcaggggaaattttaacgat-3′ | 21 | 2391 | 701 |
| Probe | TET-5′-agacacttggccttcaaagccatgtt-3′-TAMRA | 26 | 2419 | 702 |
| Reverse | 5′-caaaccaggcaatatcctgata-3′ | 22 | 2446 | 703 |

TABLE CUB
General_screening_panel_v1.4

| | Rel. | | Rel. |
|---|---|---|---|
| | Exp. (%) | | Exp. (%) |
| | Ag3250, | | Ag3250, |
| | Run | | Run |
| Tissue Name | 214693636 | Tissue Name | 214693636 |
| Adipose | 0.1 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 11.9 |
| LOXIMVI | | | |
| Melanoma* SK- | 0.6 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 0.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 0.1 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.7 |
| Breast ca. MDA- | 0.0 | Lymph Node Pool | 0.8 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.3 | Spleen Pool | 0.0 |
| Breast Pool | 100.0 | Thymus Pool | 0.0 |
| Trachea | 0.0 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.1 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 9.5 | Brain (Thalamus) Pool | 0.0 |
| Fetal Liver | 21.0 | Brain (whole) | 0.0 |
| Liver ca. HepG2 | 0.0 | Spinal Cord Pool | 0.0 |
| Kidney Pool | 17.4 | Adrenal Gland | 0.0 |
| Fetal Kidney | 6.5 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

CNS_neurodegeneration v1.0 Summary: Ag3250 Expression of this gene is low/undetectable (CTs>35) across all of the samples on this panel (data not shown).

General_screening_panel_v1.4 Summary: Ag3250 Expression of the CG57468-01 gene is highest in normal breast (CT=23.8). In addition, this gene is highly expressed in fetal/adult kidney and fetal/adult liver (CTs=26-27). Thus, expression of this gene may be used to distinguish these tissues from the other samples on this panel. Strikingly, expression of this gene is much lower in breast, kidney, and liver cancer cell lines. Therapeutic modulation of the activity of this gene or its protein product may be of benefit in the treatment of these types of cancers.

Panel 4D Summary: Ag3250 Expression of this gene is low/undetectable (CTs>35) across all of the samples on this panel (data not shown).

CV. CG59609-01: Peptidyl-Prolyl Cis-Trans Isomerase A

Expression of gene CG59609-01 was assessed using the primer-probe set Ag3494, described in Table CVA. Results of the RTQ-PCR runs are shown in Tables CVB and CVC.

TABLE CVA

| phc,1 Probe Name Ag3494 | | | | |
|---|---|---|---|---|
| | | | Start | SEQ ID |
| Primers | Sequences | Length | Position | NO: |
| Forward | 5′-ccgttctatcagccatggt-3′ | 19 | 3 | 704 |
| Probe | TET-5′-ccccaccaggttcttagacatcatcg-3′-TAMRA | 26 | 25 | 705 |
| Reverse | 5′-aaggagacacgtcccaagag-3′ | 20 | 62 | 706 |

TABLE CVB
General_screening_panel_v1.4

| | Rel. | | Rel. |
|---|---|---|---|
| | Exp. (%) | | Exp. (%) |
| | Ag3494, | | Ag3494, |
| | Run | | Run |
| Tissue Name | 217215292 | Tissue Name | 217215292 |
| Adipose | 0.0 | Renal ca.TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 25.5 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 33.4 | Colon ca.* (SW480 | 0.0 |
| carcinoma SCC-4 | | met) SW620 | |
| Testis Pool | 100.0 | Colon ca. HT29 | 0.0 |
| Prostate ca.* (bone | 0.0 | Colon ca. HCT-116 | 0.0 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. SK- | 0.0 | Colon ca. SW-48 | 0.0 |
| OV-3 | | | |
| Ovarian ca. | 0.0 | Colon Pool | 0.0 |
| OVCAR-4 | | | |
| Ovarian ca. | 0.0 | Small Intestine Pool | 0.0 |
| OVCAR-5 | | | |
| Ovarian ca. | 79.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 41.8 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 86.5 | Spleen Pool | 0.0 |
| Breast Pool | 0.0 | Thymus Pool | 0.0 |
| Trachea | 0.0 | CNS cancer | 0.0 |
| | | (glio/astro) U87-MG | |
| Lung | 0.0 | CNS cancer | 0.0 |
| | | (glio/astro) U-118-MG | |
| Fetal Lung | 0.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 40.6 | CNS cancer (glio) SF- | 0.0 |
| | | 295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 47.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 0.0 |

TABLE CVC
Panel 4D

| | Rel. Exp. (%) | | Rel. Exp. (%) |
|---|---|---|---|
| | Ag3494, Run | | Ag3494, Run |
| Tissue Name | 166441744 | Tissue Name | 166441744 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + | 0.0 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 100.0 |
| LAK cells IL-2 + IL-12 | 5.6 | Lupus Kidney | 4.6 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 0.0 |
| PBMC PWM | 0.0 | Lung fibroblast TNF | 0.0 |
| | | alpha + IL-1beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 7.7 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 1.4 |
| PMA/ionomycin | | CCD1070 IL-1beta | |
| Dendritic cells none | 0.0 | Dermal fibroblast IFN | 0.0 |
| | | gamma | |
| Dendritic cells LPS | 0.0 | Dermal fibroblast IL-4 | 0.0 |
| Dendritic cells anti- | 0.0 | IBD Colitis 2 | 26.2 |
| CD40 | | | |
| Monocytes rest | 0.0 | IBD Crohn's | 13.8 |
| Monocytes LPS | 0.0 | Colon | 17.6 |
| Macrophages rest | 0.0 | Lung | 8.1 |
| Macrophages LPS | 0.0 | Thymus | 16.5 |
| HUVEC none | 0.0 | Kidney | 0.0 |
| HUVEC starved | 0.0 | | |

CNS_neurodegeneration_v1.0 Summary: Ag3494 Expression of this gene is low/undetectable (CTs>35) across all of the samples on this panel (data not shown).

General_screening_panel_v1.4 Summary: Ag3494 Expression of the CG59609-01 gene is highest in testis (CT=34.3). In addition, low but significant expression of this gene is detected in a breast cancer cell line and an ovarian cancer cell line. Thus, expression of this gene may be used to distinguish these samples from the other samples on this panel. Furthermore, therapeutic modulation of the activity of this gene may be of benefit in the treatment of fertility, breast cancer, and ovarian cancer.

Panel 4D Summary: Ag3494 Expression of the CG59609-01 gene is highest in a liver cirrhosis sample (CT=34.3). In addition, low but significant expression of this gene is detected in samples from thymus as well as from normal and IBD colon. Thus, expression of this gene may be used to distinguish these samples from the other samples on this panel. Furthermore, therapies designed with the protein encoded for by this gene may potentially modulate liver function and play a role in the identification and treatment of inflammatory or autoimmune diseases which effect the liver including liver cirrhosis and fibrosis.

CW. CG59613-01: Proliferating Cell Nuclear Antigen

Expression of gene CG59613-01 was assessed using the primer-probe set Ag3496, described in Table CWA. Results of the RTQ-PCR runs are shown in Tables CWB and

TABLE CWA
Probe Name Ag3496

| | | | Start | SEQ ID |
|---|---|---|---|---|
| Primers | Sequences | Length | Position | NO: |
| Forward | 5′-cacataccactgtgaccacaac-3′ | 22 | 238 | 707 |
| Probe | TET-5′-cctcaccagcatgtccaaaatgctaa-3′-TAMRA | 26 | 277 | 708 |
| Reverse | 5′-tgtcttcactgccattgttgta-3′ | 22 | 305 | 709 |

TABLE CWB
General_screening_panel_v1.4

| | Rel. | | Rel. |
|---|---|---|---|
| | Exp. (%) | | Exp. (%) |
| | Ag3496, | | Ag3496, |
| | Run | | Run |
| Tissue Name | 217217871 | Tissue Name | 217217871 |
| Adipose | 9.6 | Renal ca. TK-10 | 21.8 |
| Melanoma* | 25.2 | Bladder | 11.4 |
| Hs688(A).T | | | |
| Melanoma* | 20.4 | Gastric ca. (liver met.) | 3.9 |
| Hs688(B).T | | NCI-N87 | |
| Melanoma* M14 | 0.0 | Gastric ca. KATO III | 1.1 |
| Melanoma* | 0.0 | Colon ca. SW-948 | 0.0 |
| LOXIMVI | | | |
| Melanoma* SK- | 1.5 | Colon ca. SW480 | 1.7 |
| MEL-5 | | | |
| Squamous cell | 5.4 | Colon ca.* (SW480 | 2.0 |
| carcinoma SCC-4 | | met) SW620 | |
| Testis Pool | 56.3 | Colon ca. HT29 | 3.9 |
| Prostate ca.* (bone | 2.6 | Colon ca. HCT-116 | 12.9 |
| met) PC-3 | | | |
| Prostate Pool | 37.6 | Colon ca. CaCo-2 | 8.4 |
| Placenta | 0.0 | Colon cancer tissue | 1.4 |
| Uterus Pool | 21.5 | Colon ca. SW1116 | 0.0 |
| Ovarian ca. | 18.3 | Colon ca. Colo-205 | 0.0 |
| OVCAR-3 | | | |
| Ovarian ca. SK- | 1.9 | Colon ca. SW-48 | 0.0 |
| OV-3 | | | |
| Ovarian ca. | 8.0 | Colon Pool | 29.9 |
| OVCAR-4 | | | |
| Ovarian ca. | 2.9 | Small Intestine Pool | 57.8 |
| OVCAR-5 | | | |
| Ovarian ca. | 0.0 | Stomach Pool | 38.7 |
| IGROV-1 | | | |
| Ovarian ca. | 0.0 | Bone Marrow Pool | 6.3 |
| OVCAR-8 | | | |
| Ovary | 4.9 | Fetal Heart | 6.2 |
| Breast ca. MCF-7 | 4.3 | Heart Pool | 12.3 |
| Breast ca. MDA- | 6.4 | Lymph Node Pool | 64.6 |
| MB-231 | | | |
| Breast ca. BT 549 | 4.7 | Fetal Skeletal Muscle | 31.6 |
| Breast ca. T47D | 12.9 | Skeletal Muscle Pool | 0.0 |
| Breast ca. MDA-N | 0.0 | Spleen Pool | 1.7 |
| Breast Pool | 47.6 | Thymus Pool | 47.3 |
| Trachea | 26.1 | CNS cancer | 2.3 |
| | | (glio/astro) U87-MG | |
| Lung | 7.7 | CNS cancer | 22.5 |
| | | (glio/astro) U-118-MG | |
| Fetal Lung | 82.9 | CNS cancer | 0.9 |
| | | (neuro; met) SK-N-AS | |
| Lung ca. NCI-N417 | 0.0 | CNS cancer (astro) | 1.8 |
| | | SF-539 | |
| Lung ca. LX-1 | 0.0 | CNS cancer (astro) | 35.4 |
| | | SNB-75 | |
| Lung ca. NCI-H146 | 0.0 | CNS cancer (glio) | 0.0 |
| | | SNB-19 | |
| Lung ca. SHP-77 | 3.7 | CNS cancer (glio) SF- | 9.2 |
| | | 295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 46.0 | Brain (fetal) | 23.8 |
| Lung ca. NCI-H460 | 0.5 | Brain (Hippocampus) | 0.0 |
| | | Pool | |
| Lung ca. HOP-62 | 7.5 | Cerebral Cortex Pool | 1.6 |
| Lung ca. NCI-H522 | 2.1 | Brain (Substantia | 3.8 |
| | | nigra) Pool | |
| Liver | 0.0 | Brain (Thalamus) Pool | 3.2 |
| Fetal Liver | 7.9 | Brain (whole) | 4.2 |
| Liver ca. HepG2 | 0.0 | Spinal Cord Pool | 1.9 |
| Kidney Pool | 80.7 | Adrenal Gland | 1.7 |
| Fetal Kidney | 100.0 | Pituitary gland Pool | 0.7 |
| Renal ca. 786-0 | 13.3 | Salivary Gland | 6.2 |
| Renal ca. A498 | 0.0 | Thyroid (female) | 2.6 |
| Renal ca. ACHN | 23.8 | Pancreatic ca. | 5.5 |
| | | CAPAN2 | |
| Renal ca. UO-31 | 13.7 | Pancreas Pool | 48.6 |

TABLE CWC
Panel 4D

| | Rel. Exp. (%) | | Rel. Exp. (%) |
|---|---|---|---|
| | Ag3496, Run | | Ag3496, Run |
| Tissue Name | 166441888 | Tissue Name | 166441888 |
| Secondary Th1 act | 0.0 | HUVEC IL-1beta | 0.7 |
| Secondary Th2 act | 0.7 | HUVEC IFN gamma | 1.0 |
| Secondary Tr1 act | 0.0 | HUVEC TNF alpha + | 0.0 |
| | | IFN gamma | |
| Secondary Th1 rest | 0.7 | HUVEC TNF alpha + | 0.6 |
| | | IL4 | |
| Secondary Th2 rest | 1.2 | HUVEC IL-11 | 0.4 |
| Secondary Tr1 rest | 0.4 | Lung Microvascular EC | 1.0 |
| | | none | |
| Primary Th1 act | 0.3 | Lung Microvascular EC | 1.0 |
| | | TNF alpha + IL-1beta | |
| Primary Th2 act | 1.2 | Microvascular Dermal | 0.4 |
| | | EC none | |
| Primary Tr1 act | 0.8 | Microsvasular Dermal | 0.2 |
| | | EC TNF alpha + IL-1beta | |
| Primary Th1 rest | 1.5 | Bronchial epithelium | 5.2 |
| | | TNF alpha + IL1beta | |
| Primary Th2 rest | 1.0 | Small airway epithelium | 6.6 |
| | | none | |
| Primary Tr1 rest | 1.5 | Small airway epithelium | 100.0 |
| | | TNF alpha + IL-1beta | |
| CD45RA CD4 | 2.1 | Coronery artery SMC rest | 2.6 |
| lymphocyte act | | | |
| CD45RO CD4 | 0.9 | Coronery artery SMC | 1.5 |
| lymphocyte act | | TNF alpha + IL-1beta | |
| CD8 lymphocyte act | 2.1 | Astrocytes rest | 13.1 |
| Secondary CD8 | 0.0 | Astrocytes TNF alpha + | 7.4 |
| lymphocyte rest | | IL-1beta | |
| Secondary CD8 | 0.0 | KU-812 (Basophil) rest | 4.2 |
| lymphocyte act | | | |
| CD4 lymphocyte none | 1.8 | KU-812 (Basophil) | 5.2 |
| | | PMA/ionomycin | |
| 2ry Th1/Th2/Tr1_anti- | 0.7 | CCD1106 | 17.9 |
| CD95 CH11 | | (Keratinocytes) none | |
| LAK cells rest | 0.0 | CCD1106 | 82.4 |
| | | (Keratinocytes) | |
| | | TNF alpha + IL-1beta | |
| LAK cells IL-2 | 2.3 | Liver cirrhosis | 17.2 |
| LAK cells IL-2 + IL-12 | 1.7 | Lupus Kidney | 9.3 |
| LAK cells IL-2 + IFN | 2.3 | NCI-H292 none | 0.3 |
| gamma | | | |
| LAK cells IL-2 + IL-18 | 3.1 | NCI-H292 IL-4 | 0.8 |
| LAK cells | 0.2 | NCI-H292 IL-9 | 3.0 |
| PMA/ionomycin | | | |
| NK Cells IL-2 rest | 2.7 | NCI-H292 IL-13 | 1.9 |
| Two Way MLR 3 day | 3.3 | NCI-H292 IFN gamma | 0.5 |
| Two Way MLR 5 day | 0.0 | HPAEC none | 0.3 |
| Two Way MLR 7 day | 0.2 | HPAEC TNF alpha + IL- | 0.6 |
| | | 1beta | |
| PBMC rest | 1.0 | Lung fibroblast none | 12.3 |
| PBMC PWM | 1.7 | Lung fibroblast TNF | 2.9 |
| | | alpha + IL-1beta | |
| PBMC PHA-L | 0.7 | Lung fibroblast IL-4 | 11.0 |
| Ramos (B cell) none | 0.0 | Lung fibroblast IL-9 | 5.3 |
| Ramos (B cell) | 0.0 | Lung fibroblast IL-13 | 10.4 |
| ionomycin | | | |
| B lymphocytes PWM | 0.9 | Lung fibroblast IFN | 11.9 |
| | | gamma | |
| B lymphocytes CD40L | 2.8 | Dermal fibroblast | 11.7 |
| and IL-4 | | CCD1070 rest | |
| EOL-1 dbcAMP | 0.0 | Dermal fibroblast | 5.8 |
| | | CCD1070 TNF alpha | |
| EOL-1 dbcAMP | 0.9 | Dermal fibroblast | 2.3 |
| PMA/ionomycin | | CCD1070 IL-1beta | |
| Dendritic cells none | 0.1 | Dermal fibroblast IFN | 8.8 |
| | | gamma | |
| Dendritic cells LPS | 0.7 | Dermal fibroblast IL-4 | 11.3 |
| Dendritic cells anti- | 0.6 | IBD Colitis 2 | 2.6 |
| CD40 | | | |
| Monocytes rest | 0.7 | IBD Crohn's | 1.3 |
| Monocytes LPS | 0.0 | Colon | 11.4 |
| Macrophages rest | 0.0 | Lung | 1.9 |
| Macrophages LPS | 0.0 | Thymus | 3.8 |
| HUVEC none | 1.0 | Kidney | 7.2 |
| HUVEC starved | 1.4 | | |

CNS_neurodegeneration_v1.0 Summary: Ag3496 Expression of this gene is low/undetectable (CTs>35) across all of the samples on this panel (data not shown).

General_screening_panel_v1.4 Summary: Ag3496 Expression of the CG59613-01 gene is highest in fetal and adult kidney (CTs=31). This gene is also expressed at higher levels in fetal lung (CT=31.4) than in adult lung (CT=34.8), suggesting that expression of this gene can be used to distinguish adult and fetal lung and that this gene may play a role in lung development and regeneration. Differentially higher expression in fetal tissues also occurs in brain and skeletal muscle.

In general, expression of this gene is associated with normal tissues rather than cancer cell lines. Specifically, CG59613-01 gene expression is downregulated in pancreatic, colon, gastric, renal, lung, breast and prostate cancer cell lines when compared to their respective normal tissues. Therefore, therapeutic modulation of the activity of this gene may be of benefit in the treatment of these cancers.

Among tissues with metabolic or endocrine function, this gene is expressed at low levels in pancreas, adipose, adrenal gland, fetal skeletal muscle, and the gastrointestinal tract. Therefore, therapeutic modulation of the activity of this gene may prove useful in the treatment of endocrine/metabolically related diseases, such as obesity and diabetes.

Panel 4D Summary: Ag3496 Expression of the CG59613-01 gene is highest in small airway epithelium treated with TNF alpha and IL-1 beta (CT=29.4). In addition, this gene is substantially upregulated in keratinocytes treated with TNF alpha and IL-1 beta. Low expression of this gene is also seen in lung and dermal fibroblasts independent of treatment. Therefore, therapeutics designed with the protein encoded by the transcript may reduce or eliminate symptoms caused by inflammation of the lung and skin in chronic obstructive pulmonary disease, asthma, allergy, emphysema, and psoriasis.

CX. CG59619-01: Actin, Cytoplasmic 2

Expression of gene CG59619-01 was assessed using the primer-probe set Ag3498, described in Table CXA. Results of the RTQ-PCR runs are shown in Tables CXB and CXC.

TABLE CXA
Probe Name Ag3498

| | | | Start | SEQ ID |
|---|---|---|---|---|
| Primers | Sequences | Length | Position | NO: |
| Forward | 5′-tgatatggacatccccaaag-3′ | 20 | 860 | 710 |
| Probe | TET-5′-acctgtacgccaacacagtgctgtct-3′-TAMRA | 26 | 880 | 711 |
| Reverse | 5′-atctccttctgcatcctattgg-3′ | 22 | 934 | 712 |

TABLE CXB
General_screening_panel_v1.4

| | Rel. | | Rel. |
|---|---|---|---|
| | Exp. (%) | | Exp. (%) |
| | Ag3498, | | Ag3498, |
| | Run | | Run |
| Tissue Name | 217217873 | Tissue Name | 217217873 |
| Adipose | 4.7 | Renal ca. TK-10 | 0.0 |
| Melanoma* | 0.0 | Bladder | 3.3 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 55.1 | Colon ca. HT29 | 0.0 |
| Prostate ca.* (bone | 0.0 | Colon ca. HCT-116 | 8.7 |
| met) PC-3 | | | |
| Prostate Pool | 0.0 | Colon ca. CaCo-2 | 4.9 |
| Placenta | 0.0 | Colon cancer tissue | 0.0 |
| Uterus Pool | 0.0 | Colon ca. SW1116 | 0.0 |
| Ovarian ca. | 6.6 | Colon ca. Colo-205 | 0.0 |
| OVCAR-3 | | | |
| Ovarian ca. SK- | 9.5 | Colon ca. SW-48 | 0.0 |
| OV-3 | | | |
| Ovarian ca. | 0.0 | Colon Pool | 18.3 |
| OVCAR-4 | | | |
| Ovarian ca. | 17.9 | Small Intestine Pool | 26.2 |
| OVCAR-5 | | | |
| Ovarian ca. | 0.0 | Stomach Pool | 7.2 |
| IGROV-1 | | | |
| Ovarian ca. | 0.0 | Bone Marrow Pool | 0.0 |
| OVCAR-8 | | | |
| Ovary | 2.9 | Fetal Heart | 6.4 |
| Breast ca. MCF-7 | 0.0 | Heart Pool | 11.2 |
| Breast ca. MDA- | 0.0 | Lymph Node Pool | 34.2 |
| MB-231 | | | |
| Breast ca. BT 549 | 8.4 | Fetal Skeletal Muscle | 12.9 |
| Breast ca. T47D | 2.5 | Skeletal Muscle Pool | 10.8 |
| Breast ca. MDA-N | 0.0 | Spleen Pool | 4.7 |
| Breast Pool | 13.7 | Thymus Pool | 27.0 |
| Trachea | 5.4 | CNS cancer | 0.0 |
| | | (glio/astro) U87-MG | |
| Lung | 7.0 | CNS cancer | 2.2 |
| | | (glio/astro) U-118-MG | |
| Fetal Lung | 7.9 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 3.1 | CNS cancer (glio) | 0.0 |
| | | SNB-19 | |
| Lung ca. SHP-77 | 0.0 | CNS cancer (glio) SF- | 0.0 |
| | | 295 | |
| Lung ca. A549 | 0.0 | Brain (Amygdala) | 12.9 |
| | | Pool | |
| Lung ca. NCI-H526 | 0.0 | Brain (cerebellum) | 3.2 |
| Lung ca. NCI-H23 | 0.0 | Brain (fetal) | 12.9 |
| Lung ca. NCI-H460 | 0.0 | Brain (Hippocampus) | 6.0 |
| | | Pool | |
| Lung ca. HOP-62 | 0.0 | Cerebral Cortex Pool | 4.9 |
| Lung ca. NCI-H522 | 0.0 | Brain (Substantia | 26.6 |
| | | nigra) Pool | |
| Liver | 0.0 | Brain (Thalamus) Pool | 37.9 |
| Fetal Liver | 0.0 | Brain (whole) | 0.0 |
| Liver ca. HepG2 | 0.0 | Spinal Cord Pool | 0.0 |
| Kidney Pool | 100.0 | Adrenal Gland | 0.0 |
| Fetal Kidney | 4.4 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 6.5 |
| | | CAPAN2 | |
| Renal ca. UO-31 | 0.0 | Pancreas Pool | 6.3 |

TABLE CXC
Panel 4.1D

| | Rel. Exp. (%) | | Rel. Exp. (%) |
|---|---|---|---|
| | Ag3498, Run | | Ag3498, Run |
| Tissue Name | 169839576 | Tissue Name | 169839576 |
| Secondary Th1 act | 78.5 | HUVEC IL-1beta | 11.3 |
| Secondary Th2 act | 17.0 | HUVEC IFN gamma | 12.4 |
| Secondary Tr1 act | 17.9 | HUVEC TNF alpha + | 15.7 |
| | | IFN gamma | |
| Secondary Th1 rest | 5.2 | HUVEC TNF alpha + | 12.5 |
| | | IL4 | |
| Secondary Th2 rest | 7.0 | HUVEC IL-11 | 5.4 |
| Secondary Tr1 rest | 5.5 | Lung Microvascular EC | 17.2 |
| | | none | |
| Primary Th1 act | 14.8 | Lung Microvascular EC | 7.6 |
| | | TNF alpha + IL-1beta | |
| Primary Th2 act | 16.0 | Microvascular Dermal | 8.1 |
| | | EC none | |
| Primary Tr1 act | 13.3 | Microsvasular Dermal | 8.0 |
| | | EC TNF alpha + IL-1beta | |
| Primary Th1 rest | 8.2 | Bronchial epithelium | 5.3 |
| | | TNF alpha + IL 1beta | |
| Primary Th2 rest | 7.1 | Small airway epithelium | 4.3 |
| | | none | |
| Primary Tr1 rest | 8.1 | Small airway epithelium | 6.4 |
| | | TNF alpha + IL-1beta | |
| CD45RA CD4 | 15.8 | Coronery artery SMC rest | 5.8 |
| lymphocyte act | | | |
| CD45RO CD4 | 15.9 | Coronery artery SMC | 5.9 |
| lymphocyte act | | TNF alpha + IL-1beta | |
| CD8 lymphocyte act | 19.6 | Astrocytes rest | 6.2 |
| Secondary CD8 | 14.0 | Astrocytes TNF alpha + | 4.6 |
| lymphocyte rest | | IL-1beta | |
| Secondary CD8 | 9.7 | KU-812 (Basophil) rest | 34.4 |
| lymphocyte act | | | |
| CD4 lymphocyte none | 4.3 | KU-812 (Basophil) | 31.9 |
| | | PMA/ionomycin | |
| 2ry Th1/Th2/Tr1_anti- | 6.1 | CCD1106 | 28.9 |
| CD95 CH11 | | (Keratinocytes) none | |
| LAK cells rest | 12.6 | CCD1106 | 34.2 |
| | | (Keratinocytes) | |
| | | TNF alpha + IL-1beta | |
| LAK cells IL-2 | 9.9 | Liver cirrhosis | 1.4 |
| LAK cells IL-2 + IL-12 | 12.6 | NCI-H292 none | 9.3 |
| LAK cells IL-2 + IFN | 9.4 | NCI-H292 IL-4 | 13.1 |
| gamma | | | |
| LAK cells IL-2 + IL-18 | 12.5 | NCI-H292 IL-9 | 17.7 |
| LAK cells | 13.9 | NCI-H292 IL-13 | 13.6 |
| PMA/ionomycin | | | |
| NK Cells IL-2 rest | 15.5 | NCI-H292 IFN gamma | 22.2 |
| Two Way MLR 3 day | 13.0 | HPAEC none | 4.4 |
| Two Way MLR 5 day | 14.4 | HPAEC TNF alpha + IL- | 12.4 |
| | | 1beta | |
| Two Way MLR 7 day | 8.8 | Lung fibroblast none | 5.7 |
| PBMC rest | 6.6 | Lung fibroblast TNF | 14.6 |
| | | alpha + IL-1beta | |
| PBMC PWM | 16.8 | Lung fibroblast IL-4 | 8.8 |
| PBMC PHA-L | 15.1 | Lung fibroblast IL-9 | 13.0 |
| Ramos (B cell) none | 13.0 | Lung fibroblast IL-13 | 10.2 |
| Ramos (B cell) | 11.4 | Lung fibroblast IFN | 17.8 |
| ionomycin | | gamma | |
| B lymphocytes PWM | 13.7 | Dermal fibroblast | 9.8 |
| | | CCD1070 rest | |
| B lymphocytes CD40L | 13.8 | Dermal fibroblast | 10.8 |
| and IL-4 | | CCD1070 TNF alpha | |
| EOL-1 dbcAMP | 11.1 | Dermal fibroblast | 11.2 |
| | | CCD1070 IL-1beta | |
| EOL-1 dbcAMP | 100.0 | Dermal fibroblast IFN | 19.8 |
| PMA/ionomycin | | gamma | |
| Dendritic cells none | 14.4 | Dermal fibroblast IL-4 | 9.7 |
| Dendritic cells LPS | 9.8 | Dermal Fibroblast rest | 7.7 |
| Dendritic cells anti- | 13.2 | Neutrophils TNFa + LPS | 0.7 |
| CD40 | | | |
| Monocytes rest | 8.9 | Neutrophils rest | 1.1 |
| Monocytes LPS | 32.5 | Colon | 4.4 |
| Macrophages rest | 12.6 | Lung | 5.1 |
| Macrophages LPS | 17.3 | Thymus | 7.3 |
| HUVEC none | 6.2 | Kidney | 4.9 |
| HUVEC starved | 10.0 | | |

CNS_neurodegeneration_v1.0 Summary: Ag3498 Expression of this gene is low/undetectable (CTs>35) across all of the samples on this panel (data not shown).

General_screening_panel_v1.4 Summary: Ag3498 The CG59619-01 gene is only expressed at detectable levels in the adult kidney (CT=34.2). Thus, expression of this gene can be used to distinguish kidney from the other samples on this panel. In addition, expression of this gene is much lower in fetal kidney (CT=38.7), suggesting that this gene can be used to distinguish between the fetal and adult source of this tissue. Furthermore, this gene is not expressed at detectable levels in renal cancer cell lines. Therefore, therapeutic modulation of this gene may be of use in the treatment of renal cell carcinoma.

Panel 4.1D Summary: Ag3498 Expression of the CG59619-01 gene is highest in activated eosinophils (CT=25.7), displaying 10-fold upregulation when compared to the control eosinophils. Therefore, therapies designed with the protein encoded for by this gene could block or inhibit inflammation or tissue damage due to eosinophil activation in response to asthma, ulcerative colitis and parasitic diseases.

The CG59619-01 gene is expressed at moderate levels in the majority of samples on this panel, including T cells, B cells, endothelial cells, macrophages, monocytes, dendritic cells, basophils and peripheral blood mononuclear cells, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

CY. CG59621-01: Selenide, Water Dikinase 1

Expression of gene CG59621-01 was assessed using the primer-probe set Ag3764, described in Table CYA.

TABLE CYA
Probe Name Ag3764

| | | | Start | SEQ ID |
|---|---|---|---|---|
| Primers | Sequences | Length | Position | NO: |
| Forward | 5′-catgttcagcctcatgcat-3′ | 19 | 925 | 713 |
| Probe | TET-5′-agacctcaggcggccttctgatct-3′-TAMRA | 24 | 957 | 714 |
| Reverse | 5′-ctgcttgctgacatggtaaac-3′ | 21 | 981 | 715 |

General_screening_panel_v1.4 Summary: Ag3764 Results from one experiment with the CG59621-01 gene are not included. The amp plot indicates that there were experimental difficulties with this run.

Panel 4.1D Summary: Ag3764 Expression of this gene is low/undetectable (CTs>35) across all of the samples on this panel (data not shown).

CZ. CG59625-01: Glucose Transporter Type 3

Expression of gene CG59625-01 was assessed using the primer-probe set Ag3499, described in Table CZA. Results of the RTQ-PCR runs are shown in Tables CZB and CZC.

TABLE CZA
Probe Name Ag3499

| | | | Start | SEQ ID |
|---|---|---|---|---|
| Primers | Sequences | Length | Position | NO: |
| Forward | 5′-cttcccctctgctgcttactat-3′ | 22 | 1298 | 716 |
| Probe | TET-5′-ttttattatcttcaccggcttcctca-3′-TAMRA | 26 | 1334 | 717 |
| Reverse | 5′-gaaggtaaaggccaagaaggta-3′ | 22 | 1361 | 718 |

TABLE CZB
CNS_neurodegeneration_v1.0

| | Rel. | | Rel. |
|---|---|---|---|
| | Exp. (%) | | Exp. (%) |
| | Ag3499, | Ag3499, | |
| | Run | | Run |
| Tissue Name | 210935864 | Tissue Name | 210935864 |
| AD 1 Hippo | 5.8 | Control (Path) 3 | 4.9 |
| | | Temporal Ctx | |
| AD 2 Hippo | 25.9 | Control (Path) 4 | 32.3 |
| | | Temporal Ctx | |
| AD 3 Hippo | 5.8 | AD 1 Occipital | 11.6 |
| | | Ctx | |
| AD 4 Hippo | 1.5 | AD 2 Occipital | 0.0 |
| | | Ctx (Missing) | |
| AD 5 hippo | 100.0 | AD 3 Occipital | 5.1 |
| | | Ctx | |
| AD 6 Hippo | 29.7 | AD 4 Occipital | 4.5 |
| | | Ctx | |
| Control 2 Hippo | 31.9 | AD 5 Occipital | 39.2 |
| | | Ctx | |
| Control 4 Hippo | 0.0 | AD 6 Occipital | 72.2 |
| | | Ctx | |
| Control (Path) 3 | 6.6 | Control 1 Occipital | 13.5 |
| Hippo | | Ctx | |
| AD 1 Temporal Ctx | 7.0 | Control 2 Occipital | 82.4 |
| | | Ctx | |
| AD 2 Temporal Ctx | 49.3 | Control 3 Occipital | 8.8 |
| | | Ctx | |
| AD 3 Temporal Ctx | 2.9 | Control 4 Occipital | 0.0 |
| | | Ctx | |
| AD 4 Temporal Ctx | 5.6 | Control (Path) 1 | 64.6 |
| | | Occipital Ctx | |
| AD 5 Inf Temporal | 83.5 | Control (Path) 2 | 4.0 |
| Ctx | | Occipital Ctx | |
| AD 5 SupTemporal | 37.6 | Control (Path) 3 | 4.0 |
| Ctx | | Occipital Ctx | |
| AD 6 Inf Temporal | 23.0 | Control (Path) 4 | 13.2 |
| Ctx | | Occipital Ctx | |
| AD 6 Sup Temporal | 24.8 | Control 1 Parietal | 9.9 |
| Ctx | | Ctx | |
| Control 1 Temporal | 11.0 | Control 2 Parietal | 33.2 |
| Ctx | | Ctx | |
| Control 2 Temporal | 50.7 | Control 3 Parietal | 12.1 |
| Ctx | | Ctx | |
| Control 3 Temporal | 6.0 | Control (Path) 1 | 57.8 |
| Ctx | | Parietal Ctx | |
| Control 4 Temporal | 0.1 | Control (Path) 2 | 25.2 |
| Ctx | | Parietal Ctx | |
| Control (Path) 1 | 28.9 | Control (Path) 3 | 5.6 |
| Temporal Ctx | | Parietal Ctx | |
| Control (Path) 2 | 13.9 | Control (Path) 4 | 60.3 |
| Temporal Ctx | | Parietal Ctx | |

TABLE CZC
Panel 4D

| | Rel. Exp. (%) | | Rel. Exp. (%) |
|---|---|---|---|
| | Ag3499, Run | | Ag3499, Run |
| Tissue Name | 166441940 | Tissue Name | 166441940 |
| Secondary Th1 act | 12.1 | HUVEC IL-1beta | 0.1 |
| Secondary Th2 act | 3.2 | HUVEC IFN gamma | 3.2 |
| Secondary Tr1 act | 3.6 | HUVEC TNF alpha + | 0.1 |
| | | IFN gamma | |
| Secondary Th1 rest | 7.5 | HUVEC TNF alpha + | 0.0 |
| | | IL4 | |
| Secondary Th2 rest | 5.3 | HUVEC IL-11 | 0.9 |
| Secondary Tr1 rest | 6.0 | Lung Microvascular EC | 2.0 |
| | | none | |
| Primary Th1 act | 2.4 | Lung Microvascular EC | 1.2 |
| | | TNF alpha + IL-1beta | |
| Primary Th2 act | 7.5 | Microvascular Dermal | 0.1 |
| | | EC none | |
| Primary Tr1 act | 10.1 | Microsvasular Dermal | 0.1 |
| | | EC TNF alpha + IL-1beta | |
| Primary Th1 rest | 14.9 | Bronchial epithelium | 0.3 |
| | | TNF alpha + IL-1beta | |
| Primary Th2 rest | 5.1 | Small airway epithelium | 0.3 |
| | | none | |
| Primary Tr1 rest | 5.8 | Small airway epithelium | 1.9 |
| | | TNF alpha + IL1beta | |
| CD45RA CD4 | 2.9 | Coronery artery SMC rest | 2.4 |
| lymphocyte act | | | |
| CD45RO CD4 | 12.3 | Coronery artery SMC | 1.3 |
| lymphocyte act | | TNF alpha + IL-1beta | |
| CD8 lymphocyte act | 4.1 | Astrocytes rest | 0.3 |
| Secondary CD8 | 4.3 | Astrocytes TNF alpha + | 0.5 |
| lymphocyte rest | | IL-1beta | |
| Secondary CD8 | 2.9 | KU-812 (Basophil) rest | 0.0 |
| lymphocyte act | | | |
| CD4 lymphocyte none | 3.2 | KU-812 (Basophil) | 0.3 |
| | | PMA/ionomycin | |
| 2ry Th1/Th2/Tr1_anti- | 6.4 | CCD1106 | 1.9 |
| CD95 CH11 | | (Keratinocytes) none | |
| LAK cells rest | 8.9 | CCD1106 | 30.6 |
| | | (Keratinocytes) | |
| | | TNF alpha + IL-1beta | |
| LAK cells IL-2 | 14.1 | Liver cirrhosis | 19.2 |
| LAK cells IL-2 + IL-12 | 7.1 | Lupus Kidney | 1.5 |
| LAK cells IL-2 + IFN | 8.2 | NCI-H292 none | 0.5 |
| gamma | | | |
| LAK cells IL-2 + IL-18 | 6.3 | NCI-H292 IL-4 | 0.8 |
| LAK cells | 100.0 | NCI-H292 IL-9 | 0.2 |
| PMA/ionomycin | | | |
| NK Cells IL-2 rest | 4.8 | NCI-H292 IL-13 | 0.3 |
| Two Way MLR 3 day | 7.4 | NCI-H292 IFN gamma | 0.2 |
| Two Way MLR 5 day | 7.6 | HPAEC none | 1.7 |
| Two Way MLR 7 day | 3.8 | HPAEC TNF alpha + IL- | 1.5 |
| | | 1beta | |
| PBMC rest | 0.1 | Lung fibroblast none | 3.2 |
| PBMC PWM | 10.9 | Lung fibroblast TNF | 2.7 |
| | | alpha + IL-1beta | |
| PBMC PHA-L | 9.2 | Lung fibroblast IL-4 | 4.7 |
| Ramos (B cell) none | 0.3 | Lung fibroblast IL-9 | 2.4 |
| Ramos (B cell) | 0.2 | Lung fibroblast IL-13 | 3.0 |
| ionomycin | | | |
| B lymphocytes PWM | 3.8 | Lung fibroblast IFN | 5.5 |
| | | gamma | |
| B lymphocytes CD40L | 2.6 | Dermal fibroblast | 3.7 |
| and IL-4 | | CCD1070 rest | |
| EOL-1 dbcAMP | 0.1 | Dermal fibroblast | 15.1 |
| | | CCD1070 TNF alpha | |
| EOL-1 dbcAMP | 0.6 | Dermal fibroblast | 2.5 |
| PMA/ionomycin | | CCD1070 IL-1beta | |
| Dendritic cells none | 30.4 | Dermal fibroblast IFN | 0.1 |
| | | gamma | |
| Dendritic cells LPS | 14.2 | Dermal fibroblast IL-4 | 0.1 |
| Dendritic cells anti- | 15.1 | IBD Colitis 2 | 0.0 |
| CD40 | | | |
| Monocytes rest | 5.8 | IBD Crohn's | 1.1 |
| Monocytes LPS | 12.1 | Colon | 3.3 |
| Macrophages rest | 0.3 | Lung | 7.9 |
| Macrophages LPS | 12.9 | Thymus | 0.6 |
| HUVEC none | 0.1 | Kidney | 9.4 |
| HUVEC starved | 0.1 | | |

CNS_neurodegeneration_v1.0 Summary: Ag3499 This panel confirms the expression of this gene at moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

Panel 4D Summary: Ag3499 Expression of the CG59625-01 gene is highest in PMA/ionomycin-treated lymphokine activated killer (LAK) cells (CT=24.3). Since these cells are involved in tumor immunology and tumor cell clearance, as well as virally and bacterial infected cells, therapeutic modulation of this gene product may alter the functions of these cells and lead to improvement in cancer cell killing as well as host immunity to microbial and viral infections.

This gene is also expressed at high levels in stimulated keratinocytes, dendritic cells, monocytes and macrophages, suggesting that small molecule therapeutics designed against the CG59625-01 protein could reduce or inhibit inflammation in asthma, emphysema, allergy, psoriasis, arthritis, or any other condition in which localizalion/activation of these cell types is important.

This gene is also expressed at moderate levels in a number of other cell types of significance in the immune response in health and disease.

DA. CG59887-01 and CG59887-02: Amino Acid/Metabolite Permease

Expression of gene CG59887-01 and full length clone CG59887-02 was assessed using the primer-probe set Ag4715, described in Table DAA. Please note that CG59887-02 represents a full-length physical clone of the CG59887-02 gene, validating the prediction of the gene sequence.

TABLE DAA
Probe Name Ag4715

| | | | Start | SEQ ID |
|---|---|---|---|---|
| Primers | Sequences | Length | Position | NO: |
| Forward | 5′-cgtgttgtggtggttgtttt-3′ | 20 | 1362 | 719 |
| Probe | TET-5′-actgcgcacgcgccttaacaatg-3′-TAMRA | 23 | 1383 | 720 |
| Reverse | 5′-ggctagtggtcgagcaattt-3′ | 20 | 1426 | 721 |

General_screening_panel_v1.4 Summary: Ag4715 Expression of the CG59887-01 gene is low/undetectable in all samples on this panel (CTs>35). (Data not shown.) The amp plot indicates that there is a high probability of a probe failure.

DB. CG59857-01: Rhotekin

Expression of gene CG59857-01 was assessed using the primer-probe set Ag3622, described in Table DBA. Results of the RTQ-PCR runs are shown in Tables DBB, DBC and DBD.

TABLE DBA
Probe Name Ag3622

| | | | Start | SEQ ID |
|---|---|---|---|---|
| Primers | Sequences | Length | Position | NO: |
| Forward | 5′-acatcctggaggacctgaatat-3′ | 22 | 84 | 722 |
| Probe | TET-5′-ctctacattcggcagatggcactcag-3′-TAMRA | 26 | 107 | 723 |
| Reverse | 5′-ggatctcatggtctagcttcct-3′ | 22 | 155 | 724 |

TABLE DBB
CNS_neurodegeneration_v1.0

| | Rel. | | Rel. |
|---|---|---|---|
| | Exp. (%) | | Exp. (%) |
| | Ag3622, | | Ag3622, |
| | Run | | Run |
| Tissue Name | 211005293 | Tissue Name | 211005293 |
| AD 1 Hippo | 10.7 | Control (Path) 3 | 3.9 |
| | | Temporal Ctx | |
| AD 2 Hippo | 42.9 | Control (Path) 4 | 9.2 |
| | | Temporal Ctx | |
| AD 3 Hippo | 6.9 | AD 1 Occipital Ctx | 10.1 |
| AD 4 Hippo | 13.2 | AD 2 Occipital Ctx | 0.0 |
| | | (Missing) | |
| AD 5 Hippo | 40.6 | AD 3 Occipital Ctx | 7.1 |
| AD 6 Hippo | 38.4 | AD 4 Occipital Ctx | 23.3 |
| Control 2 Hippo | 29.9 | AD 5 Occipital Ctx | 28.5 |
| Control 4 Hippo | 13.6 | AD 6 Occipital Ctx | 0.1 |
| Control (Path) 3 | 0.8 | Control 1 Occipital | 4.9 |
| Hippo | | Ctx | |
| AD 1 Temporal | 17.8 | Control 2 Occipital | 51.4 |
| Ctx | | Ctx | |
| AD 2 Temporal | 27.7 | Control 3 Occipital | 12.2 |
| Ctx | | Ctx | |
| AD 3 Temporal | 6.3 | Control 4 Occipital | 8.2 |
| Ctx | | Ctx | |
| AD 4 Temporal | 19.5 | Control (Path) 1 | 58.6 |
| Ctx | | Occipital Ctx | |
| AD 5 Inf Temporal | 100.0 | Control (Path) 2 | 10.2 |
| Ctx | | Occipital Ctx | |
| AD 5 Sup | 47.3 | Control (Path) 3 | 5.7 |
| Temporal Ctx | | Occipital Ctx | |
| AD 6 Inf Temporal | 49.0 | Control (Path) 4 | 7.9 |
| Ctx | | Occipital Ctx | |
| AD 6 Sup | 29.7 | Control 1 Parietal | 7.6 |
| Temporal Ctx | | Ctx | |
| Control 1 | 5.6 | Control 2 Parietal | 44.4 |
| Temporal Ctx | | Ctx | |
| Control 2 | 34.9 | Control 3 Parietal | 16.2 |
| Temporal Ctx | | Ctx | |
| Control 3 | 13.7 | Control (Path) 1 | 32.1 |
| Temporal Ctx | | Parietal Ctx | |
| Control 3 | 8.2 | Control (Path) 2 | 15.6 |
| Temporal Ctx | | Parietal Ctx | |
| Control (Path) 1 | 18.8 | Control (Path) 3 | 2.4 |
| Temporal Ctx | | Parietal Ctx | |
| Control (Path) 2 | 16.0 | Control (Path) 4 | 17.6 |
| Temporal Ctx | | Parietal Ctx | |

TABLE DBC
General_screening_panel_v1.4

| | Rel. Exp. (%) | Rel. Exp. (%) | | Rel. Exp. (%) | Rel. Exp. (%) |
|---|---|---|---|---|---|
| | Ag3622, Run | Ag3622, Run | | Ag3622, Run | Ag3622, Run |
| Tissue Name | 218211380 | 218307304 | Tissue Name | 218211380 | 218307304 |
| Adipose | 0.7 | 1.1 | Renal ca. TK-10 | 4.7 | 4.0 |
| Melanoma* | 4.6 | 7.6 | Bladder | 10.2 | 3.5 |
| Hs688(A).T | | | | | |
| Melanoma* | 7.2 | 6.4 | Gastric ca. (liver | 6.0 | 9.0 |
| Hs688(B).T | | | met.) NCI-N87 | | |
| Melanoma* | 29.9 | 37.9 | Gastric ca. | 7.8 | 7.7 |
| M14 | | | KATO III | | |
| Melanoma* | 15.1 | 17.2 | Colon ca. SW- | 1.9 | 1.3 |
| LOXIMVI | | | 948 | | |
| Melanoma* | 19.3 | 22.7 | Colon ca. | 11.0 | 15.1 |
| SK-MEL-5 | | | SW480 | | |
| Squamous | 0.4 | 1.5 | Colon ca.* | 5.9 | 10.4 |
| cell | | | (SW480 met) | | |
| carcinoma | | | SW620 | | |
| SCC-4 | | | | | |
| Testis Pool | 2.0 | 1.7 | Colon ca. HT29 | 5.4 | 6.8 |
| Prostate ca.* | 3.3 | 3.4 | Colon ca. HCT- | 5.6 | 6.7 |
| (bone met) | | | 116 | | |
| PC-3 | | | | | |
| Prostate Pool | 0.8 | 2.3 | Colon ca. CaCo-2 | 4.2 | 3.2 |
| Placenta | 2.2 | 3.0 | Colon cancer | 2.3 | 3.5 |
| | | | tissue | | |
| Uterus Pool | 0.8 | 0.7 | Colon ca. | 1.1 | 2.9 |
| | | | SW1116 | | |
| Ovarian ca. | 19.5 | 18.7 | Colon ca. Colo- | 1.0 | 0.3 |
| OVCAR-3 | | | 205 | | |
| Ovarian ca. | 9.0 | 8.0 | Colon ca. SW-48 | 1.2 | 1.2 |
| SK-OV-3 | | | | | |
| Ovarian ca. | 1.1 | 2.2 | Colon Pool | 16.0 | 5.8 |
| OVCAR-4 | | | | | |
| Ovarian ca. | 6.4 | 9.0 | Small Intestine | 1.7 | 3.3 |
| OVCAR-5 | | | Pool | | |
| Ovarian ca. | 10.7 | 10.7 | Stomach Pool | 2.7 | 3.8 |
| IGROV-1 | | | | | |
| Ovarian ca. | 7.1 | 20.3 | Bone Marrow | 1.2 | 1.8 |
| OVCAR-8 | | | Pool | | |
| Ovary | 3.2 | 4.3 | Fetal Heart | 5.1 | 6.5 |
| Breast ca. | 1.1 | 1.4 | Heart Pool | 1.5 | 3.0 |
| MCF-7 | | | | | |
| Breast ca. | 8.8 | 10.9 | Lymph Node | 4.9 | 6.5 |
| MDA-MB- | | | Pool | | |
| 231 | | | | | |
| Breast ca. BT | 4.7 | 8.4 | Fetal Skeletal | 69.7 | 11.3 |
| 549 | | | Muscle | | |
| Breast ca. | 17.0 | 15.2 | Skeletal Muscle | 2.7 | 1.5 |
| T47D | | | Pool | | |
| Breast ca. | 23.8 | 21.0 | Spleen Pool | 2.8 | 1.8 |
| MDA-N | | | | | |
| Breast Pool | 4.4 | 5.0 | Thymus Pool | 2.8 | 5.9 |
| Trachea | 3.8 | 5.8 | CNS cancer | 17.7 | 25.0 |
| | | | (glio/astro) U87- | | |
| | | | MG | | |
| Lung | 0.7 | 0.8 | CNS cancer | 46.0 | 57.4 |
| | | | (glio/astro) U- | | |
| | | | 118-MG | | |
| Fetal Lung | 8.2 | 9.3 | CNS cancer | 26.8 | 35.6 |
| | | | (neuro; met) SK- | | |
| | | | N-AS | | |
| Lung ca. | 0.4 | 0.5 | CNS cancer | 9.7 | 9.8 |
| NCI-N417 | | | (astro) SF-539 | | |
| Lung ca. LX-1 | 9.3 | 10.7 | CNS cancer | 22.5 | 34.4 |
| | | | (astro) SNB-75 | | |
| Lung ca. | 8.4 | 6.7 | CNS cancer | 9.0 | 13.0 |
| NCI-H146 | | | (glio) SNB-19 | | |
| Lung ca. | 7.4 | 6.4 | CNS cancer | 33.2 | 37.1 |
| SHP-77 | | | (glio) SF-295 | | |
| Lung ca. | 13.3 | 23.3 | Brain | 86.5 | 82.9 |
| A549 | | | (Amygdala) Pool | | |
| Lung ca. | 1.3 | 1.4 | Brain | 22.5 | 30.8 |
| NCI-H526 | | | (cerebellum) | | |
| Lung ca. | 5.1 | 6.0 | Brain (fetal) | 5.1 | 6.5 |
| NCI-H23 | | | | | |
| Lung ca. | 5.2 | 4.9 | Brain | 45.4 | 52.1 |
| NCI-H460 | | | (Hippocampus) | | |
| | | | Pool | | |
| Lung ca. | 5.1 | 7.4 | Cerebral Cortex | 27.4 | 40.3 |
| HOP-62 | | | Pool | | |
| Lung ca. | 6.7 | 9.8 | Brain (Substantia | 46.7 | 54.7 |
| NCI-H522 | | | nigra) Pool | | |
| Liver | 0.5 | 0.9 | Brain | 51.4 | 90.1 |
| | | | (Thalamus) Pool | | |
| Fetal Liver | 1.7 | 1.5 | Brain (whole) | 23.8 | 32.8 |
| Liver ca. | 2.9 | 4.5 | Spinal Cord Pool | 100.0 | 100.0 |
| HepG2 | | | | | |
| Kidney Pool | 5.3 | 6.4 | Adrenal Gland | 1.5 | 3.5 |
| Fetal Kidney | 7.0 | 11.7 | Pituitary gland | 0.3 | 2.5 |
| | | | Pool | | |
| Renal ca. | 2.8 | 3.0 | Salivary Gland | 1.4 | 3.2 |
| 786-0 | | | | | |
| Renal ca. | 4.9 | 5.6 | Thyroid (female) | 2.8 | 2.1 |
| A498 | | | | | |
| Renal ca. | 4.2 | 4.0 | Pancreatic ca. | 1.7 | 0.6 |
| ACHN | | | CAPAN2 | | |
| Renal ca. | 10.4 | 7.7 | Pancreas Pool | 5.2 | 6.2 |
| UO-31 | | | | | |

TABLE DBD
Panel 4.1D

| | Rel. Exp. (%) | | Rel. Exp. (%) |
|---|---|---|---|
| | Ag3622, Run | | Ag3622, Run |
| Tissue Name | 169944131 | Tissue Name | 169944131 |
| Secondary Th1 act | 0.0 | HUVEC IL-1beta | 14.7 |
| Secondary Th2 act | 2.9 | HUVEC IFN gamma | 25.2 |
| Secondary Tr1 act | 7.3 | HUVEC TNF alpha + | 2.5 |
| | | IFN gamma | |
| Secondary Th1 rest | 0.0 | HUVEC TNF alpha + | 5.8 |
| | | IL4 | |
| Secondary Th2 rest | 0.0 | HUVEC IL-11 | 19.2 |
| Secondary Tr1 rest | 0.0 | Lung Microvascular EC | 49.0 |
| | | none | |
| Primary Th1 act | 6.5 | Lung Microvascular EC | 11.3 |
| | | TNF alpha + IL-1beta | |
| Primary Th2 act | 0.0 | Microvascular Dermal | 7.7 |
| | | EC none | |
| Primary Tr1 act | 0.0 | Microsvasular Dermal | 10.9 |
| | | EC TNF alpha + IL-1beta | |
| Primary Th1 rest | 0.0 | Bronchial epithelium | 21.5 |
| | | TNF alpha + IL1beta | |
| Primary Th2 rest | 0.0 | Small airway epithelium | 7.2 |
| | | none | |
| Primary Tr1 rest | 0.0 | Small airway epithelium | 27.9 |
| | | TNF alpha + IL-1beta | |
| CD45RA CD4 | 0.8 | Coronery artery SMC rest | 22.5 |
| lymphocyte act | | | |
| CD45RO CD4 | 3.5 | Coronery artery SMC | 31.4 |
| lymphocyte act | | TNF alpha + IL-1beta | |
| CD8 lymphocyte act | 1.8 | Astrocytes rest | 17.0 |
| Secondary CD8 | 1.6 | Astrocytes TNF alpha + | 25.3 |
| lymphocyte rest | | IL-1beta | |
| Secondary CD8 | 0.2 | KU-812 (Basophil) rest | 8.0 |
| lymphocyte act | | | |
| CD4 lymphocyte none | 0.0 | KU-812 (Basophil) | 3.4 |
| | | PMA/ionomycin | |
| 2ry Th1/Th2/Tr1_anti- | 4.2 | CCD1106 | 44.8 |
| CD95 CH11 | | (Keratinocytes) none | |
| LAK cells rest | 0.4 | CCD1106 | 61.6 |
| | | (Keratinocytes) | |
| | | TNF alpha + IL-1beta | |
| LAK cells IL-2 | 6.0 | Liver cirrhosis | 0.0 |
| LAK cells IL-2 + IL-12 | 0.0 | NCI-H292 none | 5.3 |
| LAK cells IL-2 + IFN | 1.5 | NCI-H292 IL-4 | 9.9 |
| gamma | | | |
| LAK cells IL-2 + IL-18 | 0.0 | NCI-H292 IL-9 | 14.4 |
| LAK cells | 0.0 | NCI-H292 IL-13 | 1.4 |
| PMA/ionomycin | | | |
| NK Cells IL-2 rest | 13.9 | NCI-H292 IFN gamma | 11.4 |
| Two Way MLR 3 day | 3.6 | HPAEC none | 20.9 |
| Two Way MLR 5 day | 3.8 | HPAEC TNF alpha + IL- | 10.2 |
| | | 1beta | |
| Two Way MLR 7 day | 0.0 | Lung fibroblast none | 38.2 |
| PBMC rest | 1.7 | Lung fibroblast TNF | 25.0 |
| | | alpha + IL-1beta | |
| PBMC PWM | 3.6 | Lung fibroblast IL-4 | 35.8 |
| PBMC PHA-L | 1.3 | Lung fibroblast IL-9 | 100.0 |
| Ramos (B cell) none | 0.2 | Lung fibroblast IL-13 | 69.7 |
| Ramos (B cell) | 1.1 | Lung fibroblast IFN | 58.2 |
| ionomycin | | gamma | |
| B lymphocytes PWM | 0.5 | Dermal fibroblast | 39.2 |
| | | CCD1070 rest | |
| B lymphocytes CD40L | 7.9 | Dermal fibroblast | 5.7 |
| and IL-4 | | CCD1070 TNF alpha | |
| EOL-1 dbcAMP | 0.0 | Dermal fibroblast | 17.4 |
| | | CCD1070 IL-1beta | |
| EOL-1 dbcAMP | 0.0 | Dermal fibroblast IFN | 31.2 |
| PMA/ionomycin | | gamma | |
| Dendritic cells none | 9.9 | Dermal fibroblast IL-4 | 44.8 |
| Dendritic cells LPS | 2.4 | Dermal Fibroblasts rest | 19.1 |
| Dendritic cells anti- | 24.7 | Neutrophils TNFa + LPS | 0.0 |
| CD40 | | | |
| Monocytes rest | 0.0 | Neutrophils rest | 0.0 |
| Monocytes LPS | 6.3 | Colon | 10.3 |
| Macrophages rest | 8.8 | Lung | 13.6 |
| Macrophages LPS | 3.4 | Thymus | 2.4 |
| HUVEC none | 11.1 | Kidney | 23.8 |
| HUVEC starved | 28.5 | | |

CNS_neurodegeneration_v1.0 Summary: Ag3622 This panel confirms the expression of the CG59857-01 gene at significant levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3622 Two experiments with the same probe and primer set show highest expression of the CG59857-01 gene in spinal cord samples (CTs=26-28). In addition, high levels of expression of this gene are seen in brain derived tissue, including samples from amygdala, hippocampus, substantia nigra, thalamus, cerebellum, cerebral cortex, and CNS cancer cell lines. Therefore, expression of this gene could be used to distinguish between brain derived samples and other samples used in this panel. Furthermore, this gene may play a role in central nervous system disorders such as Alzheinier's disease, Parkinson's disease, epilepsy, multiple sclerosis, schizophrenia and depression.

Significant expression is also detected in fetal skeletal muscle (CTs=27-31). Interestingly, this gene is expressed at much higher levels in fetal when compared to adult skeletal muscle (CTs=32-34). This observation suggests that expression of this gene can be used to distinguish fetal from adult skeletal muscle. In addition, the relative overexpression of this gene in fetal skeletal muscle suggests that the protein product may enhance muscular growth or development in the fetus and thus may also act in a regenerative capacity in the adult. Therefore, therapeutic modulation of the protein encoded by this gene could be useful in treatment of muscle related diseases. More specifically, treatment of weak or dystrophic muscle with the protein encoded by this gene could restore muscle mass or function.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

Panel 4.1D Summary: Ag3622 Highest expression of the CG59857-01 gene is seen in IL-9/IL-13 treated lung fibroblasts (CT=31). In addition, significant expression is seen in clusters of treated and untreated lung and dermal fibroblasts, epithelium and endothelium. Therefore, modulation of the gene product with a functional therapeutic may lead to the alteration of functions associated with these cell types and lead to improvement of the symptoms of patients suffering from autoimmune and inflammatory diseases such as asthma, allergies, and psoriasis.

DC. CG59855-01 and CG59855-02: ATP Synthase Subunit C

Expression of gene CG59855-01 and full length clone CG59855-02 was assessed using the primer-probe set Ag3621, described in Table DCA. Results of the RTQ-PCR runs are shown in Tables DCB and DCC. Please note that CG59855-02 represents a full-length physical clone of the CG59855-02 gene, validating the prediction of the gene sequence.

TABLE DCA
Probe Name Ag3621

| | | | Start | SEQ ID |
|---|---|---|---|---|
| Primers | Sequences | Length | Position | NO: |
| Forward | 5′-gggtctaatcaggcctgtgt-3′ | 20 | 73 | 725 |
| Probe | TET-5′-tgccttctccttgaatagcccagaga-3′-TAMRA | 26 | 94 | 726 |
| Reverse | 5′-ctgctgtaggaaggctgtttag-3′ | 22 | 126 | 727 |

TABLE DCB
General_screening_panel_v1.4

| | Rel. Exp. | | Rel. Exp. |
|---|---|---|---|
| | (%) Ag3621, | | (%) Ag3621, |
| | Run | | Run |
| Tissue Name | 217702346 | Tissue Name | 217702346 |
| Adipose | 0.0 | Renal ca. TK-10 | 0.0 |
| Melanoma* | 0.0 | Bladder | 7.5 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 35.1 | Colon ca. HT29 | 0.0 |
| Prostate ca.* (bone | 0.0 | Colon ca. HCT-116 | 0.0 |
| met) PC-3 | | | |
| Prostate Pool | 3.5 | Colon ca. CaCo-2 | 0.0 |
| Placenta | 2.9 | Colon cancer tissue | 0.0 |
| Uterus Pool | 5.9 | Colon ca. SW1116 | 0.0 |
| Ovarian ca. | 0.0 | Colon ca. Colo-205 | 0.0 |
| OVCAR-3 | | | |
| Ovarian ca. SK- | 47.6 | Colon ca. SW-48 | 0.0 |
| OV-3 | | | |
| Ovarian ca. | 0.0 | Colon Pool | 57.0 |
| OVCAR-4 | | | |
| Ovarian ca. | 0.0 | Small Intestine Pool | 42.9 |
| OVCAR-5 | | | |
| Ovarian ca. | 0.0 | Stomach Pool | 7.5 |
| IGROV-1 | | | |
| Ovarian ca. | 0.0 | Bone Marrow Pool | 7.0 |
| OVCAR-8 | | | |
| Ovary | 12.9 | Fetal Heart | 0.0 |
| Breast ca. MCF-7 | 0.0 | Heart Pool | 0.0 |
| Breast ca. MDA- | 0.0 | Lymph Node Pool | 8.1 |
| MB-231 | | | |
| Breast ca. BT 549 | 0.0 | Fetal Skeletal Muscle | 13.2 |
| Breast ca. T47D | 0.0 | Skeletal Muscle Pool | 0.0 |
| Breast ca. MDA-N | 0.0 | Spleen Pool | 27.4 |
| Breast Pool | 49.0 | Thymus Pool | 25.9 |
| Trachea | 46.3 | CNS cancer | 0.0 |
| | | (glio/astro) U87-MG | |
| Lung | 38.4 | CNS cancer | 0.0 |
| | | (glio/astro) U-118-MG | |
| Fetal Lung | 100.0 | CNS cancer | 0.0 |
| | | (neuro; met) SK-N-AS | |
| Lung ca. NCI-N417 | 0.0 | CNS cancer (astro) | 0.0 |
| | | SF-539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 0.0 | CNS cancer (glio) | 0.0 |
| | | SF-295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 6.7 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain (Substantia | 0.0 |
| | | nigra) Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 6.1 |
| Kidney Pool | 57.8 | Adrenal Gland | 0.0 |
| Fetal Kidney | 10.7 | Pituitary gland Pool | 12.4 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. | 0.0 |
| | | CAPAN2 | |
| Renal ca. UO-31 | 0.0 | Pancreas Pool | 63.3 |

TABLE DCC
Panel 4.1D

| | Rel. Exp. (%) | | Rel. Exp. (%) |
|---|---|---|---|
| | Ag3621, Run | | Ag3621, Run |
| Tissue Name | 169944096 | Tissue Name | 169944096 |
| Secondary Th1 act | 1.0 | HUVEC IL-1beta | 0.0 |
| Secondary Th2 act | 0.0 | HUVEC IFN gamma | 1.0 |
| Secondary Tr1 act | 16.5 | HUVEC TNF alpha + | 0.0 |
| | | IFN gamma | |
| Secondary Th1 rest | 7.5 | HUVEC TNF alpha + | 0.0 |
| | | IL4 | |
| Secondary Th2 rest | 2.6 | HUVEC IL-11 | 7.8 |
| Secondary Tr1 rest | 0.0 | Lung Microvascular EC | 1.0 |
| | | none | |
| Primary Th1 act | 0.0 | Lung Microvascular EC | 6.7 |
| | | TNF alpha + IL-1beta | |
| Primary Th2 act | 0.0 | Microvascular Dermal | 0.0 |
| | | EC none | |
| Primary Tr1 act | 0.0 | Microsvasular Dermal | 0.0 |
| | | EC TNF alpha + IL-1beta | |
| Primary Th1 rest | 0.0 | Bronchial epithelium | 0.0 |
| | | TNF alpha + IL-1beta | |
| Primary Th2 rest | 1.5 | Small airway epithelium | 0.0 |
| | | none | |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 1.1 |
| Secondary CD8 | 0.0 | Astrocytes TNF alpha + | 0.9 |
| lymphocyte rest | | IL-1beta | |
| Secondary CD8 | 0.0 | KU-812 (Basophil) rest | 0.0 |
| lymphocyte act | | | |
| CD4 lymphocyte none | 8.6 | KU-812 (Basophil) | 0.0 |
| | | PMA/ionomycin | |
| 2ry Th1/Th2/Tr1_anti- | 0.0 | CCD1106 | 0.0 |
| CD95 CH11 | | (Keratinocytes) none | |
| LAK cells rest | 0.0 | CCD1106 | 0.0 |
| | | (Keratinocytes) | |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 4.5 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 1.5 |
| LAK cells | 2.3 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 1.3 | HPAEC none | 2.9 |
| Two Way MLR 5 day | 0.0 | HPAEC TNF alpha + IL- | 2.3 |
| | | 1beta | |
| Two Way MLR 7 day | 0.9 | Lung fibroblast none | 5.1 |
| PBMC rest | 1.0 | Lung fibroblast TNF | 0.0 |
| | | alpha + IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 9.0 |
| | | CCD1070 rest | |
| B lymphocytes CD40L | 0.0 | Dermal fibroblast | 1.1 |
| and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 2.0 | Dermal fibroblast IL-4 | 2.8 |
| Dendritic cells LPS | 17.6 | Dermal Fibroblasts rest | 0.0 |
| Dendritic cells anti- | 2.7 | Neutrophils TNFa + LPS | 0.0 |
| CD40 | | | |
| Monocytes rest | 100.0 | Neutrophils rest | 20.0 |
| Monocytes LPS | 6.1 | Colon | 0.8 |
| Macrophages rest | 2.8 | Lung | 0.0 |
| Macrophages LPS | 0.0 | Thymus | 0.0 |
| HUVEC none | 0.0 | Kidney | 9.3 |
| HUVEC starved | 0.0 | | |

CNS_neurodegeneration_v1.0 Summary: Ag3621 Expression of the CG59855-01 gene is low/undetectable in all samples on this panel (CTs>35). (Data not shown.)

General_screening_panel_v1.4 Summary: Ag3621 Expression of the CG59855-01 gene is restricted to samples from fetal lung and adult pancrease(CTs=34.5-35). Thus, expression of this gene can be used to distinguish this sample from other samples in the panel.

The CG59855-01 gene encodes a homologue of ATP synthase subunit c, mitochondrial precursor. Subunit c is an intrinsic membrane component of ATP synthase, and in mammals it is encoded by two expressed nuclear genes, P1 and P2. Both genes encode the same mature c subunit, but the mitochondrial import pre-sequences in the precursors of subunit c are different (ref. 1). Each ATP synthase complex has multiple copies of subunit C. The mitochondrial ATP synthase uses energy derived from a proton gradient to synthesize ATP. The structure of this complex has been referred to as a ‘lollipop,’ as the soluble F1 catalytic unit is attached to the mitochondrial inner membrane via the F0 unit containing subunit c. F0 subunit C transports protons across the mitochondrial inner membrane to the F1-ATPase (ref. 2).

Subunit C of the F0 region of the ATP synthase complex of the inner mitochondrial membrane is found in high concentrations in lysosomes in late infantile neuronal ceroid lipofuscinosis (Batten's disease). Kominami et al. (1995, Ref 3) found marked delay of degradation of subunit C in patient fibroblasts with no significant differences between control and patient cells with regard to degradation of cytochrome oxidase subunit IV. Furthermore, accumulation of labeled subunit C in the mitochondrial fraction was detected before Lysosomal appearance of the radiolabeled subunit, suggesting to the authors a specific failure in the degradation of subunit C after its normal inclusion in mitochondria and its consequent accumulation in lysosomes. Jolly (1995, ref 4) reported that subunit C represents more than 50% of the accumulated metabolites in the ovine form of the disease and also accumulates significantly in late infantile and juvenile forms of the human disease and several other animal forms. The author suggested that the extreme hydrophobicity and lipophilicity of subunit C may be in part responsible.

### REFERENCES

1. Dyer M R, Walker J E. (1993) Sequences of members of the human gene family for the c subunit of mitochondrial ATP synthase. Biochem J 293 (Pt 1):51-64

2. OMIM 603192

3. Kominami E, Ezaki J, Wolfe L S. (1995) New insight into lysosomal protein storage disease: delayed catabolism of ATP synthase subunit c in Batten disease. Neuroctiem Res 20(11):1305-9

4. Jolly R D. (1995) Batten disease (ceroid-lipofuscinosis): the enigma of subunit c of mitochondrial ATP synthase accumulation. Neurochem Res 20(11):1301-4

Panel 4.1D Summary: Ag3621 Expression of the CG59855-01 gene is exclusively seen in resting monocytes (CT=32). Thus, expression of this gene can be used to distinguish this sample from other samples in the panel. In addition, expression of this gene in monocytes suggests a role for the gene product in their function as antigen-presenting cells. This suggests that antibodies or small molecule therapeutics that block the function of this protein nay be useful as anti-inflammatory therapeutics for the treatment of autoimmune and inflammatory diseases and for the treatment of immunosupressed individuals.

DD. CG59807-01: Nuclear Hormone Receptor/Zinc Finger

Expression of gene CG59807-01 was assessed using the primer-probe set Ag3591, described in Table DDA. Results of the RTQ-PCR runs are shown in Tables DDB and DDC.

TABLE DDA

Probe Name Ag3591

| | | | | |
|---|---|---|---|---|
| | | | Start | SEQ ID |
| Primers | Sequences | Length | Position | NO: |

| | | | | |
|---|---|---|---|---|
| Forward | 5′-cactgctccacttttgtcttg-3′ | 21 | 1195 | 728 |
| Probe | TET-5′-cataaaaggacccacacaggagaaaa-3′-TAMRA | 26 | 1216 | 729 |
| Reverse | 5′-cttttccacattctttgcattc-3′ | 22 | 1249 | 730 |

TABLE DDB
General_screening_panel_v1.4

| | Rel. Exp. | | Rel. Exp. |
|---|---|---|---|
| | (%) Ag3591, | | (%) Ag3591, |
| | Run | | Run |
| Tissue Name | 217479278 | Tissue Name | 217479278 |
| Adipose | 13.9 | Renal ca. TK-10 | 22.2 |
| Melanoma* | 18.6 | Bladder | 27.5 |
| Hs688(A).T | | | |
| Melanoma* | 17.6 | Gastric ca. (liver met.) | 100.0 |
| Hs688(B).T | | NCI-N87 | |
| Melanoma* M14 | 22.8 | Gastric ca. KATO III | 63.7 |
| Melanoma* | 12.5 | Colon ca. SW-948 | 4.9 |
| LOXIMVI | | | |
| Melanoma* SK- | 10.4 | Colon ca. SW480 | 41.2 |
| MEL-5 | | | |
| Squamous cell | 27.9 | Colon ca.* (SW480 | 17.1 |
| carcinoma SCC-4 | | met) SW620 | |
| Testis Pool | 14.9 | Colon ca. HT29 | 19.3 |
| Prostate ca.* (bone | 51.4 | Colon ca. HCT-116 | 52.9 |
| met) PC-3 | | | |
| Prostate Pool | 12.7 | Colon ca. CaCo-2 | 13.5 |
| Placenta | 8.7 | Colon cancer tissue | 7.4 |
| Uterus Pool | 5.0 | Colon ca. SW1116 | 11.3 |
| Ovarian ca. | 29.9 | Colon ca. Colo-205 | 8.3 |
| OVCAR-3 | | | |
| Ovarian ca. SK- | 49.7 | Colon ca. SW-48 | 3.2 |
| OV-3 | | | |
| Ovarian ca. | 9.1 | Colon Pool | 18.8 |
| OVCAR-4 | | | |
| Ovarian ca. | 14.6 | Small Intestine Pool | 19.2 |
| OVCAR-5 | | | |
| Ovarian ca. | 26.2 | Stomach Pool | 15.1 |
| IGROV-1 | | | |
| Ovarian ca. | 14.4 | Bone Marrow Pool | 7.5 |
| OVCAR-8 | | | |
| Ovary | 12.7 | Fetal Heart | 24.5 |
| Breast ca. MCF-7 | 9.5 | Heart Pool | 10.9 |
| Breast ca. MDA- | 42.0 | Lymph Node Pool | 33.9 |
| MB-231 | | | |
| Breast ca. BT 549 | 66.0 | Fetal Skeletal Muscle | 24.0 |
| Breast ca. T47D | 40.3 | Skeletal Muscle Pool | 19.9 |
| Breast ca. MDA-N | 13.7 | Spleen Pool | 30.4 |
| Breast Pool | 25.5 | Thymus Pool | 30.6 |
| Trachea | 14.5 | CNS cancer | 19.2 |
| | | (glio/astro) U87-MG | |
| Lung | 14.6 | CNS cancer | 51.1 |
| | | (glio/astro) U-118-MG | |
| Fetal Lung | 72.7 | CNS cancer | 49.0 |
| | | (neuro; met) SK-N-AS | |
| Lung ca. NCI-N417 | 2.6 | CNS cancer (astro) | 18.7 |
| | | SF-539 | |
| Lung ca. LX-1 | 21.2 | CNS cancer (astro) | 48.6 |
| | | SNB-75 | |
| Lung ca. NCI-H146 | 24.5 | CNS cancer (glio) | 26.6 |
| | | SNB-19 | |
| Lung ca. SHP-77 | 33.7 | CNS cancer (glio) | 80.1 |
| | | SF-295 | |
| Lung ca. A549 | 16.6 | Brain (Amygdala) | 10.6 |
| | | Pool | |
| Lung ca. NCI-H526 | 14.7 | Brain (cerebellum) | 65.5 |
| Lung ca. NCI-H23 | 35.4 | Brain (fetal) | 50.0 |
| Lung ca. NCI-H460 | 33.0 | Brain (Hippocampus) | 15.3 |
| | | Pool | |
| Lung ca. HOP-62 | 10.4 | Cerebral Cortex Pool | 18.6 |
| Lung ca. NCI-H522 | 30.6 | Brain (Substantia | 19.9 |
| | | nigra) Pool | |
| Liver | 2.0 | Brain (Thalamus) Pool | 26.6 |
| Fetal Liver | 21.6 | Brain (whole) | 25.9 |
| Liver ca. HepG2 | 16.4 | Spinal Cord Pool | 16.3 |
| Kidney Pool | 27.7 | Adrenal Gland | 25.0 |
| Fetal Kidney | 28.1 | Pituitary gland Pool | 8.2 |
| Renal ca. 786-0 | 25.2 | Salivary Gland | 7.0 |
| Renal ca. A498 | 12.9 | Thyroid (female) | 9.2 |
| Renal ca. ACHN | 23.5 | Pancreatic ca. | 12.5 |
| | | CAPAN2 | |
| Renal ca. UO-31 | 32.1 | Pancreas Pool | 16.8 |

TABLE DDC
Panel 4.1D

| | Rel. Exp. (%) | | Rel. Exp. (%) |
|---|---|---|---|
| | Ag3591, Run | | Ag3591, Run |
| Tissue Name | 169908857 | Tissue Name | 169908857 |
| Secondary Th1 act | 38.4 | HUVEC IL-1beta | 24.7 |
| Secondary Th2 act | 37.4 | HUVEC IFN gamma | 23.5 |
| Secondary Tr1 act | 48.6 | HUVEC TNF alpha + | 17.3 |
| | | IFN gamma | |
| Secondary Th1 rest | 22.7 | HUVEC TNF alpha + | 20.6 |
| | | IL4 | |
| Secondary Th2 rest | 34.9 | HUVEC IL-11 | 19.6 |
| Secondary Tr1 rest | 35.1 | Lung Microvascular EC | 36.6 |
| | | none | |
| Primary Th1 act | 47.0 | Lung Microvascular EC | 39.5 |
| | | TNF alpha + IL-1beta | |
| Primary Th2 act | 43.5 | Microvascular Dermal | 22.1 |
| | | EC none | |
| Primary Tr1 act | 50.3 | Microsvasular Dermal | 21.9 |
| | | EC TNF alpha + IL-1beta | |
| Primary Th1 rest | 43.5 | Bronchial epithelium | 35.4 |
| | | TNF alpha + IL-1beta | |
| Primary Th2 rest | 40.6 | Small airway epithelium | 14.0 |
| | | none | |
| Primary Tr1 rest | 51.1 | Small airway epithelium | 31.0 |
| | | TNF alpha + IL-1beta | |
| CD45RA CD4 | 22.2 | Coronery artery SMC rest | 14.5 |
| lymphocyte act | | | |
| CD45RO CD4 | 38.4 | Coronery artery SMC | 12.0 |
| lymphocyte act | | TNF alpha + IL-1beta | |
| CD8 lymphocyte act | 39.8 | Astrocytes rest | 24.1 |
| Secondary CD8 | 22.5 | Astrocytes TNF alpha + | 20.0 |
| lymphocyte rest | | IL-1beta | |
| Secondary CD8 | 31.0 | KU-812 (Basophil) rest | 57.8 |
| lymphocyte act | | | |
| CD4 lymphocyte none | 15.8 | KU-812 (Basophil) | 98.6 |
| | | PMA/ionomycin | |
| 2ry Th1/Th2/Tr1_anti- | 57.8 | CCD1106 | 28.7 |
| CD95 CH11 | | (Keratinocytes) none | |
| LAK cells rest | 28.9 | CCD1106 | 24.1 |
| | | (Keratinocytes) | |
| | | TNF alpha + IL-1beta | |
| LAK cells IL-2 | 44.4 | Liver cirrhosis | 8.6 |
| LAK cells IL-2 + IL-12 | 35.4 | NCI-H292 none | 40.1 |
| LAK cells IL-2 + IFN | 44.8 | NCI-H292 IL-4 | 80.7 |
| gamma | | | |
| LAK cells IL-2 + IL-18 | 52.5 | NCI-H292 IL-9 | 79.0 |
| LAK cells | 18.3 | NCI-H292 IL-13 | 100.0 |
| PMA/ionomycin | | | |
| NK Cells IL-2 rest | 44.1 | NCI-H292 IFN gamma | 100.0 |
| Two Way MLR 3 day | 42.0 | HPAEC none | 21.6 |
| Two Way MLR 5 day | 25.7 | HPAEC TNF alpha + IL- | 32.8 |
| | | 1beta | |
| Two Way MLR 7 day | 20.4 | Lung fibroblast none | 28.5 |
| PBMC rest | 11.2 | Lung fibroblast TNF | 13.9 |
| | | alpha + IL-1beta | |
| PBMC PWM | 21.3 | Lung fibroblast IL-4 | 28.1 |
| PBMC PHA-L | 24.7 | Lung fibroblast IL-9 | 49.3 |
| Ramos (B cell) none | 61.1 | Lung fibroblast IL-13 | 37.9 |
| Ramos (B cell) | 66.9 | Lung fibroblast IFN | 29.5 |
| ionomycin | | gamma | |
| B lymphocytes PWM | 24.1 | Dermal fibroblast | 29.9 |
| | | CCD1070 rest | |
| B lymphocytes CD40L | 56.6 | Dermal fibroblast | 45.1 |
| and IL-4 | | CCD1070 TNF alpha | |
| EOL-1 dbcAMP | 69.7 | Dermal fibroblast | 11.0 |
| | | CCD1070 IL-1beta | |
| EOL-1 dbcAMP | 54.0 | Dermal fibroblast IFN | 8.7 |
| PMA/ionomycin | | gamma | |
| Dendritic cells none | 26.6 | Dermal fibroblast IL-4 | 29.7 |
| Dendritic cells LPS | 10.2 | Dermal Fibroblast rest | 12.2 |
| Dendritic cells anti- | 23.2 | Neutrophils TNFa + LPS | 1.3 |
| CD40 | | | |
| Monocytes rest | 12.2 | Neutrophils rest | 3.3 |
| Monocytes LPS | 16.5 | Colon | 8.8 |
| Macrophages rest | 18.6 | Lung | 33.4 |
| Macrophages LPS | 8.1 | Thymus | 82.9 |
| HUVEC none | 17.4 | Kidney | 23.8 |
| HUVEC starved | 27.2 | | |

General_screening_panel_v1.4 Summary: Ag3591 Highest expression of the CG59807-01 gene is detected in the gastric cancer cell line (CT=28). In addition, high expression of this gene is seen in samples derived from CNS cancer, colon cancer, breast cancer, ovarian cancer, prostate cancer cell lines (CTs=28-31). Therefore, therapeutic modulation of the activity of this gene or its protein product, through the use of small molecule drugs, protein therapeutics or antibodies, might be beneficial in the treatment of these cancers.

In addition, expression of this gene is higher in fetal liver (CT=31) as compared to the corresponding adult tissues (CTs=34). Thus, expression of this gene can be used to distinguish between the fetal and adults source of this tissue.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

This gene is also expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag3591 Highest expression of the CG59807-01 gene is detected in treated mucoepidermoid NCI-H292 cells. In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

DE. CG59805-01: Nuclear Hormone Receptor/Zinc Finger

Expression of gene CG59805-01 was assessed using the primer-probe set Ag3590, described in Table DEA. Results of the RTQ-PCR runs are shown in Tables DEB, DEC and DED.

TABLE DEA
Probe Name Ag3590

| | | | Start | SEQ ID |
|---|---|---|---|---|
| Primers | Sequences | Length | Position | NO: |
| Forward | 5′-atgagtgcagtgaatgtggaa-3′ | 21 | 1620 | 731 |
| Probe | TET-5′-cttcagtcgcagctcgtccctcact-3′-TAMRA | 25 | 1645 | 732 |
| Reverse | 5′-atttctcccagtatgcatcctt-3′ | 22 | 1678 | 733 |

TABLE DEB
CNS_neurodegeneration_v1.0

| | Rel. Exp. | | Rel. Exp. |
|---|---|---|---|
| | (%) Ag3590, | | (%) Ag3590, |
| | Run | | Run |
| Tissue Name | 211006692 | Tissue Name | 211006692 |
| AD 1 Hippo | 16.7 | Control (Path) 3 | 5.8 |
| | | Temporal Ctx | |
| AD 2 Hippo | 22.1 | Control (Path) 4 | 42.6 |
| | | Temporal Ctx | |
| AD 3 Hippo | 8.2 | AD 1 Occipital Ctx | 17.7 |
| AD 4 Hippo | 7.9 | AD 2 Occipital Ctx | 0.0 |
| | | (Missing) | |
| AD 5 Hippo | 73.2 | AD 3 Occipital Ctx | 3.9 |
| AD 6 Hippo | 87.7 | AD 4 Occipital Ctx | 23.3 |
| Control 2 Hippo | 25.7 | AD 5 Occipital Ctx | 32.5 |
| Control 4 Hippo | 17.0 | AD 6 Occipital Ctx | 31.2 |
| Control (Path) 3 | 7.4 | Control 1 Occipital | 5.2 |
| Hippo | | Ctx | |
| AD 1 Temporal | 25.2 | Control 2 Occipital | 39.2 |
| Ctx | | Ctx | |
| AD 2 Temporal | 34.2 | Control 3 Occipital | 20.0 |
| Ctx | | Ctx | |
| AD 3 Temporal | 10.1 | Control 4 Occipital | 8.1 |
| Ctx | | Ctx | |
| AD 4 Temporal | 28.3 | Control (Path) 1 | 80.7 |
| Ctx | | Occipital Ctx | |
| AD 5 Inf Temporal | 71.7 | Control (Path) 2 | 11.1 |
| Ctx | | Occipital Ctx | |
| AD 5 Sup | 35.4 | Control (Path) 3 | 6.1 |
| Temporal Ctx | | Occipital Ctx | |
| AD 6 Inf Temporal | 98.6 | Control (Path) 4 | 17.9 |
| Ctx | | Occipital Ctx | |
| AD 6 Sup | 100.0 | Control 1 Parietal | 8.8 |
| Temporal Ctx | | Ctx | |
| Control 1 | 8.2 | Control 2 Parietal | 39.0 |
| Temporal Ctx | | Ctx | |
| Control 2 | 29.1 | Control 3 Parietal | 18.3 |
| Temporal Ctx | | Ctx | |
| Control 3 | 17.3 | Control (Path) 1 | 55.5 |
| Temporal Ctx | | Parietal Ctx | |
| Control 3 | 10.2 | Control (Path) 2 | 26.8 |
| Temporal Ctx | | Parietal Ctx | |
| Control (Path) 1 | 50.7 | Control (Path) 3 | 7.4 |
| Temporal Ctx | | Parietal Ctx | |
| Control (Path) 2 | 34.6 | Control (Path) 4 | 46.0 |
| Temporal Ctx | | Parietal Ctx | |

TABLE DEC
General_screening_panel_v1.4

| | Rel. Exp. | | Rel. Exp. |
|---|---|---|---|
| | (%) Ag3590, | | (%) Ag3590, |
| | Run | | Run |
| Tissue Name | 217474417 | Tissue Name | 217474417 |
| Adipose | 12.8 | Renal ca. TK-10 | 28.9 |
| Melanoma* | 33.7 | Bladder | 28.5 |
| Hs688(A).T | | | |
| Melanoma* | 25.3 | Gastric ca. (liver met.) | 82.4 |
| Hs688(B).T | | NCI-N87 | |
| Melanoma* M14 | 16.3 | Gastric ca. KATO III | 24.5 |
| Melanoma* | 19.6 | Colon ca. SW-948 | 5.8 |
| LOXIMVI | | | |
| Melanoma* SK- | 16.5 | Colon ca. SW480 | 28.1 |
| MEL-5 | | | |
| Squamous cell | 29.5 | Colon ca.* (SW480 | 10.7 |
| carcinoma SCC-4 | | met) SW620 | |
| Testis Pool | 16.0 | Colon ca. HT29 | 13.6 |
| Prostate ca.* (bone | 57.4 | Colon ca. HCT-116 | 26.2 |
| met) PC-3 | | | |
| Prostate Pool | 14.6 | Colon ca. CaCo-2 | 28.5 |
| Placenta | 7.7 | Colon cancer tissue | 13.0 |
| Uterus Pool | 6.0 | Colon ca. SW1116 | 4.8 |
| Ovarian ca. | 18.9 | Colon ca. Colo-205 | 3.4 |
| OVCAR-3 | | | |
| Ovarian ca. SK- | 38.2 | Colon ca. SW-48 | 2.3 |
| OV-3 | | | |
| Ovarian ca. | 15.4 | Colon Pool | 27.0 |
| OVCAR-4 | | | |
| Ovarian ca. | 17.4 | Small Intestine Pool | 24.1 |
| OVCAR-5 | | | |
| Ovarian ca. | 12.2 | Stomach Pool | 14.3 |
| IGROV-1 | | | |
| Ovarian ca. | 12.5 | Bone Marrow Pool | 11.7 |
| OVCAR-8 | | | |
| Ovary | 14.2 | Fetal Heart | 21.8 |
| Breast ca. MCF-7 | 29.1 | Heart Pool | 10.7 |
| Breast ca. MDA- | 28.3 | Lymph Node Pool | 29.5 |
| MB-231 | | | |
| Breast ca. BT 549 | 100.0 | Fetal Skeletal Muscle | 10.7 |
| Breast ca. T47D | 34.4 | Skeletal Muscle Pool | 14.7 |
| Breast ca. MDA-N | 12.5 | Spleen Pool | 26.2 |
| Breast Pool | 40.6 | Thymus Pool | 26.8 |
| Trachea | 16.3 | CNS cancer | 32.3 |
| | | (glio/astro) U87-MG | |
| Lung | 4.6 | CNS cancer | 57.0 |
| | | (glio/astro) U-118-MG | |
| Fetal Lung | 66.4 | CNS cancer | 33.2 |
| | | (neuro; met) SK-N-AS | |
| Lung ca. NCI-N417 | 2.0 | CNS cancer (astro) | 14.4 |
| | | SF-539 | |
| Lung ca. LX-1 | 15.9 | CNS cancer (astro) | 46.7 |
| | | SNB-75 | |
| Lung ca. NCI-H146 | 3.3 | CNS cancer (glio) | 14.9 |
| | | SNB-19 | |
| Lung ca. SHP-77 | 24.5 | CNS cancer (glio) | 82.9 |
| | | SF-295 | |
| Lung ca. A549 | 17.2 | Brain (Amygdala) | 7.2 |
| | | Pool | |
| Lung ca. NCI-H526 | 5.4 | Brain (cerebellum) | 13.2 |
| Lung ca. NCI-H23 | 26.4 | Brain (fetal) | 26.8 |
| Lung ca. NCI-H460 | 22.5 | Brain (Hippocampus) | 10.3 |
| | | Pool | |
| Lung ca. HOP-62 | 10.6 | Cerebral Cortex Pool | 14.6 |
| Lung ca. NCI-H522 | 18.9 | Brain (Substantia | 13.9 |
| | | nigra) Pool | |
| Liver | 1.6 | Brain (Thalamus) Pool | 18.2 |
| Fetal Liver | 26.1 | Brain (whole) | 16.5 |
| Liver ca. HepG2 | 19.8 | Spinal Cord Pool | 9.1 |
| Kidney Pool | 43.5 | Adrenal Gland | 30.8 |
| Fetal Kidney | 26.2 | Pituitary gland Pool | 7.3 |
| Renal ca. 786-0 | 26.1 | Salivary Gland | 7.7 |
| Renal ca. A498 | 19.8 | Thyroid (female) | 6.5 |
| Renal ca. ACHN | 14.6 | Pancreatic ca. | 13.3 |
| | | CAPAN2 | |
| Renal ca. UO-31 | 27.0 | Pancreas Pool | 25.2 |

TABLE DED
Panel 4.1D

| | Rel. Exp. (%) | | Rel. Exp. (%) |
|---|---|---|---|
| | Ag3590, Run | | Ag3590, Run |
| Tissue Name | 169908854 | Tissue Name | 169908854 |
| Secondary Th1 act | 33.9 | HUVEC IL-1beta | 31.0 |
| Secondary Th2 act | 42.6 | HUVEC IFN gamma | 30.6 |
| Secondary Tr1 act | 52.1 | HUVEC TNF alpha + | 18.9 |
| | | IFN gamma | |
| Secondary Th1 rest | 20.3 | HUVEC TNF alpha + | 18.2 |
| | | IL4 | |
| Secondary Th2 rest | 28.1 | HUVEC IL-11 | 17.4 |
| Secondary Tr1 rest | 26.6 | Lung Microvascular EC | 39.5 |
| | | none | |
| Primary Th1 act | 44.1 | Lung Microvascular EC | 54.0 |
| | | TNF alpha + IL-1beta | |
| Primary Th2 act | 38.7 | Microvascular Dermal | 25.9 |
| | | EC none | |
| Primary Tr1 act | 45.7 | Microsvasular Dermal | 36.6 |
| | | EC TNF alpha + IL-1beta | |
| Primary Th1 rest | 23.3 | Bronchial epithelium | 51.1 |
| | | TNF alpha + IL-1beta | |
| Primary Th2 rest | 27.7 | Small airway epithelium | 22.1 |
| | | none | |
| Primary Tr1 rest | 30.4 | Small airway epithelium | 33.0 |
| | | TNF alpha + IL-1beta | |
| CD45RA CD4 | 24.8 | Coronery artery SMC rest | 38.4 |
| lymphocyte act | | | |
| CD45RO CD4 | 42.9 | Coronery artery SMC | 29.1 |
| lymphocyte act | | TNF alpha + IL-1beta | |
| CD8 lymphocyte act | 39.2 | Astrocytes rest | 34.9 |
| Secondary CD8 | 32.8 | Astrocytes TNF alpha + | 26.2 |
| lymphocyte rest | | IL-1beta | |
| Secondary CD8 | 19.5 | KU-812 (Basophil) rest | 56.6 |
| lymphocyte act | | | |
| CD4 lymphocyte none | 37.4 | KU-812 (Basophil) | 100.0 |
| | | PMA/ionomycin | |
| 2ry Th1/Th2/Tr1_anti- | 39.8 | CCD1106 | 29.3 |
| CD95 CH11 | | (Keratinocytes) none | |
| LAK cells rest | 43.8 | CCD1106 | 34.9 |
| | | (Keratinocytes) | |
| | | TNF alpha + IL-1beta | |
| LAK cells IL-2 | 38.2 | Liver cirrhosis | 12.9 |
| LAK cells IL-2 + IL-12 | 39.5 | NCI-H292 none | 22.2 |
| LAK cells IL-2 + IFN | 50.3 | NCI-H292 IL-4 | 38.7 |
| gamma | | | |
| LAK cells IL-2 + IL-18 | 51.1 | NCI-H292 IL-9 | 41.2 |
| LAK cells | 50.0 | NCI-H292 IL-13 | 36.1 |
| PMA/ionomycin | | | |
| NK Cells IL-2 rest | 41.5 | NCI-H292 IFN gamma | 56.3 |
| Two Way MLR 3 day | 50.7 | HPAEC none | 19.3 |
| Two Way MLR 5 day | 28.5 | HPAEC TNF alpha + IL- | 50.3 |
| | | 1beta | |
| Two Way MLR 7 day | 19.5 | Lung fibroblast none | 37.6 |
| PBMC rest | 33.2 | Lung fibroblast TNF | 24.0 |
| | | alpha + IL-1beta | |
| PBMC PWM | 25.7 | Lung fibroblast IL-4 | 49.3 |
| PBMC PHA-L | 16.3 | Lung fibroblast IL-9 | 56.6 |
| Ramos (B cell) none | 42.3 | Lung fibroblast IL-13 | 43.5 |
| Ramos (B cell) | 33.4 | Lung fibroblast IFN | 42.9 |
| ionomycin | | gamma | |
| B lymphocytes PWM | 24.3 | Dermal fibroblast | 38.7 |
| | | CCD1070 rest | |
| B lymphocytes CD40L | 36.9 | Dermal fibroblast | 43.8 |
| and IL-4 | | CCD1070 TNF alpha | |
| EOL-1 dbcAMP | 40.3 | Dermal fibroblast | 22.8 |
| | | CCD1070 IL-1beta | |
| EOL-1 dbcAMP | 37.9 | Dermal fibroblast IFN | 12.3 |
| PMA/ionomycin | | gamma | |
| Dendritic cells none | 33.7 | Dermal fibroblast IL-4 | 55.1 |
| Dendritic cells LPS | 18.9 | Dermal Fibroblasts rest | 23.2 |
| Dendritic cells anti- | 26.8 | Neutrophils TNFa + LPS | 1.4 |
| CD40 | | | |
| Monocytes rest | 33.7 | Neutrophils rest | 16.5 |
| Monocytes LPS | 31.4 | Colon | 8.8 |
| Macrophages rest | 19.8 | Lung | 41.2 |
| Macrophages LPS | 6.6 | Thymus | 82.9 |
| HUVEC none | 16.0 | Kidney | 30.8 |
| HUVEC starved | 20.0 | | |

CNS_neurodegeneration_v1.0 Summary: Ag3590 This panel confirms the expression of the CG59805-01 gene at low levels in the brain in an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3590 Highest expression of the CG59805-01 gene is detected in one of the breast cancer cell line BT 549 (CT=26). In addition, expression of this gene is high in CNS cancer, gastric cancer, and prostate cancer cell lines. Therefore, expression of this gene can be used to distinguish these samples from other samples in this panel and it can be used as marker for detection of these cancers. Furthermore, therapeutic modulation of the activity of the protein encoded by this gene may be beneficial in the treatment of these cancers.

Among tissues with metabolic or endocrine function, this gene is expressed at high to moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases. such as obesity and diabetes.

In addtion, this gene is expressed at high levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

Panel 4.1D Summary: Ag3590 Highest expression of the CG59805-01 gene is detected in PMA/ionomycin treated Ku-812 (basophil) cells (CT=29). In addition, this gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

DF. CG59928-01: Novel Universal Stress (USP) Domain Containg Protein

Expression of gene CG59928-01 was assessed using the primer-probe set Ag3636, described in Table DFA. Please note that this sequence is represented by a full length clone.

TABLE DFA
Probe Name Ag3636

| | | | Start | SEQ ID |
|---|---|---|---|---|
| Primers | Sequences | Length | Position | NO: |
| Forward | 5′-gaagccttcgacaagctgat-3′ | 20 | 1268 | 734 |
| Probe | TET-5′-atcgatagagcacaggcccacctgtt-3′-TAMRA | 26 | 1301 | 735 |
| Reverse | 5′-gatgacttcctcggcaaaac-3′ | 20 | 1332 | 736 |

CNS_neurodegeneration_v1.0 Summary: Ag3636 Expression of the CG59928-01 gene is low/undetectable in all samples on this panel (CTs>35). (Data not shown.) The amp plot indicates that there is a high probability of a probe failure.

General_screening_panel_v1.4 Summary: Ag3636 Expression of the CG59928-01 gene is low/undetectable in all samples on this panel (CTs>35). (Data not shown.) The amp plot indicates that there is a high probability of a probe failure.

Panel 4.1D Summary: Ag3636 Expression of the CG59928-01 gene is low/undetectable in all samples on this panel (CTs>35). (Data not shown.) The amp plot indicates that there is a high probability of a probe failure.

DG. CG59947-01: Voltage-Gated Potassium Channel Protein KV3.3

Expression of gene CG59947-01 was assessed using the primer-probe set Ag3635, described in Table DGA. Results of the RTQ-PCR runs are shown in Tables DGB, DGC, DGD and DGE.

TABLE DGA
Probe Name Ag3635

| | | | Start | SEQ ID |
|---|---|---|---|---|
| Primers | Sequences | Length | Position | NO: |
| Forward | 5′-tacttcaagaacatccccattg-3′ | 22 | 1326 | 737 |
| Probe | TET-5′-ctgtggtcaccatgacgaccctg-3′-TAMRA | 23 | 1360 | 738 |
| Reverse | 5′-tcttggggtacatgtctccata-3′ | 22 | 1386 | 739 |

TABLE DGB
CNS_neurodegeneration_v1.0

| | Rel. Exp. | | Rel. Exp. |
|---|---|---|---|
| | (%) Ag3635, | | (%) Ag3635, |
| | Run | | Run |
| Tissue Name | 211020704 | Tissue Name | 211020704 |
| AD 1 Hippo | 8.7 | Control (Path) 3 | 2.8 |
| | | Temporal Ctx | |
| AD 2 Hippo | 14.3 | Control (Path) 4 | 23.8 |
| | | Temporal Ctx | |
| AD 3 Hippo | 5.1 | AD 1 Occipital | 0.9 |
| | | Ctx | |
| AD 4 Hippo | 4.8 | AD 2 Occipital | 0.0 |
| | | Ctx (Missing) | |
| AD 5 hippo | 100.0 | AD 3 Occipital | 4.2 |
| | | Ctx | |
| AD 6 Hippo | 23.7 | AD 4 Occipital | 13.0 |
| | | Ctx | |
| Control 2 Hippo | 22.4 | AD 5 Occipital | 18.4 |
| | | Ctx | |
| Control 4 Hippo | 4.9 | AD 6 Occipital | 47.3 |
| | | Ctx | |
| Control (Path) 3 | 2.7 | Control 1 Occipital | 2.0 |
| Hippo | | Ctx | |
| AD 1 Temporal Ctx | 6.8 | Control 2 Occipital | 99.3 |
| | | Ctx | |
| AD 2 Temporal Ctx | 18.3 | Control 3 Occipital | 11.3 |
| | | Ctx | |
| AD 3 Temporal Ctx | 4.6 | Control 4 Occipital | 2.6 |
| | | Ctx | |
| AD 4 Temporal Ctx | 11.5 | Control (Path) 1 | 80.7 |
| | | Occipital Ctx | |
| AD 5 Inf Temporal | 68.8 | Control (Path) 2 | 10.4 |
| Ctx | | Occipital Ctx | |
| AD 5 SupTemporal | 23.5 | Control (Path) 3 | 2.5 |
| Ctx | | Occipital Ctx | |
| AD 6 Inf Temporal | 21.6 | Control (Path) 4 | 18.4 |
| Ctx | | Occipital Ctx | |
| AD 6 Sup Temporal | 31.0 | Control 1 Parietal | 3.9 |
| Ctx | | Ctx | |
| Control 1 Temporal | 3.8 | Control 2 Parietal | 20.2 |
| Ctx | | Ctx | |
| Control 2 Temporal | 30.8 | Control 3 Parietal | 20.3 |
| Ctx | | Ctx | |
| Control 3 Temporal | 8.3 | Control (Path) 1 | 66.4 |
| Ctx | | Parietal Ctx | |
| Control 4 Temporal | 4.9 | Control (Path) 2 | 21.2 |
| Ctx | | Parietal Ctx | |
| Control (Path) 1 | 46.7 | Control (Path) 3 | 1.8 |
| Temporal Ctx | | Parietal Ctx | |
| Control (Path) 2 | 16.8 | Control (Path) 4 | 51.4 |
| Temporal Ctx | | Parietal Ctx | |

TABLE DGC
Panel 2.2

| | Rel. Exp. | | Rel. Exp. |
|---|---|---|---|
| | (%) Ag3635, | | (%) Ag3635, |
| | Run | | Run |
| Tissue Name | 173764364 | Tissue Name | 173764364 |
| Normal Colon | 5.4 | Kidney Margin | 100.0 |
| | | (OD04348) | |
| Colon cancer | 5.0 | Kidney malignant | 5.0 |
| (OD06064) | | cancer (OD06204B) | |
| Colon Margin | 3.0 | Kidney normal | 19.2 |
| (OD06064) | | adjacent tissue | |
| | | (OD06204E) | |
| Colon cancer | 1.0 | Kidney Cancer | 5.7 |
| (OD06159) | | (OD04450-01) | |
| Colon Margin | 2.8 | Kidney Margin | 23.3 |
| (OD06159) | | (OD04450-03) | |
| Colon cancer | 2.0 | Kidney Cancer | 1.2 |
| (OD06297-04) | | 8120613 | |
| Colon Margin | 5.0 | Kidney Margin | 23.5 |
| (OD06297-05) | | 8120614 | |
| CC Gr.2 ascend colon | 4.4 | Kidney Cancer | 3.6 |
| (ODO3921) | | 9010320 | |
| CC Margin | 0.7 | Kidney Margin | 12.9 |
| (ODD3921) | | 9010321 | |
| Colon cancer | 1.8 | Kidney Cancer | 13.3 |
| metastasis (OD06104) | | 8120607 | |
| Lung Margin | 0.6 | Kidney Margin | 16.3 |
| (OD06104) | | 8120608 | |
| Colon mets to lung | 1.6 | Normal Uterus | 6.3 |
| (OD04451-01) | | | |
| Lung Margin | 6.1 | Uterine Cancer | 20.3 |
| (OD04451-02) | | 064011 | |
| Normal Prostate | 8.2 | Normal Thyroid | 6.7 |
| Prostate Cancer | 2.2 | Thyroid Cancer | 4.8 |
| (OD04410) | | 064010 | |
| Prostate Margin | 3.4 | Thyroid Cancer | 33.7 |
| (OD04410) | | A302152 | |
| Normal Ovary | 5.4 | Thyroid Margin | 7.0 |
| | | A302153 | |
| Ovarian cancer | 9.9 | Normal Breast | 23.7 |
| (OD06283-03) | | | |
| Ovarian Margin | 5.6 | Breast Cancer | 3.2 |
| (OD06283-07) | | (OD04566) | |
| Ovarian Cancer | 8.0 | Breast Cancer 1024 | 63.3 |
| 064008 | | | |
| Ovarian cancer | 13.1 | Breast Cancer | 8.7 |
| (OD06145) | | (OD04590-01) | |
| Ovarian Margin | 15.7 | Breast Cancer Mets | 4.0 |
| (OD06145) | | (OD04590-03) | |
| Ovarian cancer | 24.7 | Breast Cancer | 78.5 |
| (OD06455-03) | | Metastasis | |
| | | (OD04655-05) | |
| Ovarian Margin | 3.5 | Breast Cancer | 16.3 |
| (OD06455-07) | | 064006 | |
| Normal Lung | 8.2 | Breast Cancer | 5.2 |
| | | 9100266 | |
| Invasive poor diff. | 5.4 | Breast Margin | 5.4 |
| lung adeno | | 9100265 | |
| (ODO4945-01) | | | |
| Lung Margin | 7.7 | Breast Cancer | 2.2 |
| (ODO4945-03) | | A209073 | |
| Lung Malignant | 3.7 | Breast Margin | 20.3 |
| Cancer (OD03126) | | A2090734 | |
| Lung Margin | 3.1 | Breast cancer | 9.1 |
| (OD03126) | | (OD06083) | |
| Lung Cancer | 8.8 | Breast cancer node | 7.6 |
| (OD05014A) | | metastasis | |
| | | (OD06083) | |
| Lung Margin | 12.6 | Normal Liver | 7.4 |
| (OD05014B) | | | |
| Lung cancer | 11.0 | Liver Cancer 1026 | 4.5 |
| (OD06081) | | | |
| Lung Margin | 6.7 | Liver Cancer 1025 | 9.7 |
| (OD06081) | | | |
| Lung Cancer | 1.0 | Liver Cancer | 7.4 |
| (OD04237-01) | | 6004-T | |
| Lung Margin | 10.4 | Liver Tissue | 7.4 |
| (OD04237-02) | | 6004-N | |
| Ocular Melanoma | 5.1 | Liver Cancer | 5.2 |
| Metastasis | | 6005-T | |
| Ocular Melanoma | 6.1 | Liver Tissue | 8.8 |
| Margin (Liver) | | 6005-N | |
| Melanoma Metastasis | 1.6 | Liver Cancer | 5.9 |
| | | 064003 | |
| Melanoma Margin | 5.1 | Normal Bladder | 6.1 |
| (Lung) | | | |
| Normal Kidney | 11.3 | Bladder Cancer | 4.6 |
| | | 1023 | |
| Kidney Ca, Nuclear | 51.8 | Bladder Cancer | 7.9 |
| grade 2 (OD04338) | | A302173 | |
| Kidney Margin | 4.7 | Normal Stomach | 8.6 |
| (OD04338) | | | |
| Kidney Ca Nuclear | 24.5 | Gastric Cancer | 1.2 |
| grade 1/2 (OD04339) | | 9060397 | |
| Kidney Margin | 21.5 | Stomach Margin | 4.3 |
| (OD04339) | | 9060396 | |
| Kidney Ca, Clear cell | 2.3 | Gastric Cancer | 1.9 |
| type (OD04340) | | 9060395 | |
| Kidney Margin | 14.8 | Stomach Margin | 6.8 |
| (OD04340) | | 9060394 | |
| Kidney Ca, Nuclear | 1.1 | Gastric Cancer | 3.1 |
| grade 3 (OD04348) | | 064005 | |

TABLE DGE
Panel CNS_1

| | Rel. Exp. (%) | | Rel. Exp. |
|---|---|---|---|
| | Ag3635, Run | | Ag3635, Run |
| Tissue Name | 171648701 | Tissue Name | 171648701 |
| BA4 Control | 16.7 | BA17 PSP | 27.0 |
| BA4 Control2 | 39.2 | BA17 PSP2 | 8.9 |
| BA4 | 5.0 | Sub Nigra Control | 1.1 |
| Alzheimer's2 | | | |
| BA4 Parkinson's | 28.5 | Sub Nigra Control2 | 16.5 |
| BA4 | 100.0 | Sub Nigra | 4.1 |
| Parkinson's2 | | Alzheimer's2 | |
| BA4 | 24.7 | Sub Nigra | 26.1 |
| Huntington's | | Parkinson's2 | |
| BA4 | 9.7 | Sub Nigra | 25.3 |
| Huntington's2 | | Huntington's | |
| BA4 PSP | 11.7 | Sub Nigra | 9.3 |
| | | Huntington's2 | |
| BA4 PSP2 | 36.6 | Sub Nigra PSP2 | 2.4 |
| BA4 Depression | 14.9 | Sub Nigra | 2.0 |
| | | Depression | |
| BA4 | 8.8 | Sub Nigra | 4.5 |
| Depression2 | | Depression2 | |
| BA7 Control | 25.3 | Glob Palladus | 3.3 |
| | | Control | |
| BA7 Control2 | 42.6 | Glob Palladus | 4.9 |
| | | Control2 | |
| BA7 | 4.5 | Glob Palladus | 4.5 |
| Alzheimer's2 | | Alzheimer's | |
| BA7 Parkinson's | 10.4 | Glob Palladus | 1.2 |
| | | Alzheimer's2 | |
| BA7 | 29.5 | Glob Palladus | 21.6 |
| Parkinson's2 | | Parkinson's | |
| BA7 | 25.3 | Glob Palladus | 2.0 |
| Huntington's | | Parkinson's2 | |
| BA7 | 12.9 | Glob Palladus PSP | 1.6 |
| Huntington's2 | | | |
| BA7 PSP | 30.6 | Glob Palladus PSP2 | 2.1 |
| BA7 PSP2 | 12.1 | Glob Palladus | 1.4 |
| | | Depression | |
| BA7 Depression | 10.1 | Temp Pole Control | 7.3 |
| BA9 Control | 15.0 | Temp Pole Control2 | 27.4 |
| BA9 Control2 | 47.0 | Temp Pole | 4.2 |
| | | Alzheimer's | |
| BA9 Alzheimer's | 4.7 | Temp Pole | 2.5 |
| | | Alzheimer's2 | |
| BA9 | 9.2 | Temp Pole | 15.1 |
| Alzheimer's2 | | Parkinson's | |
| BA9 Parkinson's | 28.9 | Temp Pole | 14.2 |
| | | Parkinson's2 | |
| BA9 | 34.4 | Temp Pole | 19.2 |
| Parkinson's2 | | Huntington's | |
| BA9 | 24.1 | Temp Pole PSP | 3.4 |
| Huntington's | | | |
| BA9 | 9.5 | Temp Pole PSP2 | 3.0 |
| Huntington's2 | | | |
| BA9 PSP | 10.2 | Temp Pole | 4.8 |
| | | Depression2 | |
| BA9 PSP2 | 6.2 | Cing Gyr Control | 20.9 |
| BA9 Depression | 5.6 | Cing Gyr Control2 | 25.9 |
| BA9 | 8.3 | Cing Gyr | 5.6 |
| Depression2 | | Alzheimer's | |
| BA17 Control | 33.2 | Cing Gyr | 6.3 |
| | | Alzheimer's2 | |
| BA17 Control2 | 64.6 | Cing Gyr | 9.1 |
| | | Parkinson's | |
| BA17 | 7.2 | Cing Gyr | 13.1 |
| Alzheimer's2 | | Parkinson's2 | |
| BA17 | 18.3 | Cing Gyr | 22.1 |
| Parkinson's | | Huntington's | |
| BA17 | 35.1 | Cing Gyr | 6.0 |
| Parkinson's2 | | Huntington's2 | |
| BA17 | 32.1 | Cing Gyr PSP | 6.5 |
| Huntington's | | | |
| BA17 | 8.8 | Cing Gyr PSP2 | 2.6 |
| Huntington's2 | | | |
| BA17 | 7.3 | Cing Gyr | 2.6 |
| Depression | | Depression | |
| BA17 | 25.7 | Cing Gyr | 6.1 |
| Depression2 | | Depression2 | |

CNS_neurodegeneration_v1.0 Summary: Ag3635 This panel confirms the expression of CG59947-01 gene at low levels in the brain in an independent group of individuals. However, no differential expression of this gene was detected between Alzheimer's diseased postmortem brains and those of non-demented controls in this experiment. This gene encodes a potassium channel protein homolog. The significant levels of expression in the brain may indicate a role for this protein in signal processing in the central nervous system.

### REFERENCES

1. Rudy B, Chow A, Lau D, Amarillo Y, Ozaita A, Saganich M, Moreno H, Nadal M S, Hernandez-Pineda R, Hernandez-Cruz A, Erisir A, Leonard C, Vega-Saenz de Miera E.

2. Contributions of Kv3 channels to neuronal excitability. Ann NY Acad Sci Apr. 30, 1999;868:304-43

General_screening_panel_v1.4 Summary: Ag3635 Results from one experiment with the CG59947-01 gene are not included. The amp plot indicates that there were experimental difficulties with this run.

Panel 2.2 Summary: Ag3635 Highest expression of the CG59447-01 gene is seen in normal kidney tissue adjacent to a tumor (CT=28). In addition, expression appears to be higher in normal kidney tissue than in the adjacent tumor in six out of nine matched pairs. Conversely expression appears to be higher in breast cancer than in matched normal breast tissue. Thus, expression of this gene could be used to differentiate between these samples and other samples on this panel and as a marker for kidney and breast cancers. Furthermore, therapeutic modulation of the expression or function of this protein may be effective in the treatment of breast and kidney cancer.

Panel 4.1D Summary: Ag3635 This gene is expressed at high to moderate levels in a wide range of cell types of significance in the immune response in health and disease, with highest expression in anti CD40 dendritic cells (CT=28.1). Other cells that express this protein include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel CNS_{—}1 Summary: Ag3635 Expression in this panel confirms expression of the CG59947-01 gene in the brain. Please see Panel CNS_neurodegeneration_v1.0 for discussion of utility of this gene in the central nervous system.

DH. CG59938-01: Arylsulfatase

Expression of gene CG59938-01 was assessed using the primer-probe set Ag3634, described in Table DHA.

TABLE DHA
Probe Name Ag3634

| | | | Start | SEQ ID |
|---|---|---|---|---|
| Primers | Sequences | Length | Position | NO: |
| Forward | 5′-agccaatgaaagaggagaaagt-3′ | 22 | 870 | 740 |
| Probe | TET-5′-cttccctcatgctgaaggaggcactt-3′-TAMRA | 26 | 894 | 741 |
| Reverse | 5′-cccttttgtacctttcaatgaa-3′ | 22 | 923 | 742 |

CNS_neurodegeneration_v1.0 Summary: Ag3634 Expression of the CG55938-01 gene is low/undetectable in all samples on this panel (CTs>35). (Data not shown.)

General_screening_panel_v1.4 Summary: Ag3634 Expression of the CG55938-01 gene is low/undetectable in all samples on this panel (CTs>35). (Data not shown.)

Panel 2.2 Summary: Ag3634 Expression of the CG55938-01 gene is low/undetectable in all samples on this panel (CTs>35). (Data not shown.)

Panel 4.1D Summary: Ag3634 Expression of the CG55938-01 gene is low/undetectable in all samples on this panel (CTs>35). (Data not shown.)

DI. CG!59746-01: Ubiquitin Carboxyl-Terminal Hydrolase

Expression of gene CG59746-01 was assessed using the primer-probe set Ag3574, described in Table DIA.

TABLE DIA
Probe Name Ag3574

| | | | Start | SEQ ID |
|---|---|---|---|---|
| Primers | Sequences | Length | Position | NO: |
| Forward | 5′-agcacaacacagaaggaaatca-3′ | 22 | 461 | 743 |
| Probe | TET-5′-tcattccacaaagttgatgagaaatca-3′-TAMRA | 27 | 491 | 744 |
| Reverse | 5′-gtcccacttccttttgctatct-3′ | 22 | 534 | 745 |

CNS_neurodegeneration_v1.0 Summary: Ag3574 Expression of the CG59746-01 gene is low/undetectable in all samples on this panel (CTs>35). (Data not shown.)

General_screening_panel_v1.4 Summary: Ag3574 Expression of the CG59746-01 gene is low/undetectable in all samples on this panel (CTs>35). (Data not shown.)

Panel 2.2 Summary: Ag3574 Expression of the CG59746-01 gene is low/undetectable in all samples on this panel (CTs>35). (Data not shown.)

Panel 4.1D Summary: Ag3574 Expression of the CG59746-01 gene is low/undetectable in all samples on this panel (CTs>35). (Data not shown.)

Panel CNS_{—}1 Summary: Ag3574 Expression of the CG59746-01 gene is low/undetectable in all samples on this panel (CTs>35). (Data not shown.)

DJ. CG8613-01: Inositol 1,4,5-Trisphosphate 3-Kinase Isoenzyme

Expression of gene CG88613-01 was assessed using the primer-probe set Ag3647, described in Table DJA. Results of the RTQ-PCR runs are shown in Tables DJB, DJC and DJD.

TABLE DJA
Probe Name Ag3647

| | | | Start | SEQ ID |
|---|---|---|---|---|
| Primer | Sequences | Length | Position | NO: |
| Forward | 5′-actggagcaggtgacaaaagt-3′ | 21 | 1731 | 766 |
| Probe | TET-5′-accacgtcatcctgcaaaagtacgtg-3′-TAMRA | 26 | 1775 | 767 |
| Reverse | 5′-cagagcttcacgaagttcttct-3′ | 22 | 1809 | 768 |

TABLE DJB
CNS_neurodegeneration_v1.0

| | Rel. Exp. | | Rel. Exp. |
|---|---|---|---|
| | (%) Ag3647, | | (%) Ag3647, |
| | Run | | Run |
| Tissue Name | 211019283 | Tissue Name | 211019283 |
| AD 1 Hippo | 44.8 | Control (Path) 3 | 16.3 |
| | | Temporal Ctx | |
| AD 2 Hippo | 28.1 | Control (Path) 4 | 20.7 |
| | | Temporal Ctx | |
| AD 3 Hippo | 10.2 | AD 1 Occipital | 30.1 |
| | | Ctx | |
| AD 4 Hippo | 10.7 | AD 2 Occipital | 0.0 |
| | | Ctx (Missing) | |
| AD 5 hippo | 98.6 | AD 3 Occipital | 17.1 |
| | | Ctx | |
| AD 6 Hippo | 75.3 | AD 4 Occipital | 17.9 |
| | | Ctx | |
| Control 2 Hippo | 33.4 | AD 5 Occipital | 39.8 |
| | | Ctx | |
| Control 4 Hippo | 29.1 | AD 6 Occipital | 42.6 |
| | | Ctx | |
| Control (Path) 3 | 14.9 | Control 1 Occipital | 8.0 |
| Hippo | | Ctx | |
| AD 1 Temporal Ctx | 19.5 | Control 2 Occipital | 67.4 |
| | | Ctx | |
| AD 2 Temporal Ctx | 29.1 | Control 3 Occipital | 29.3 |
| | | Ctx | |
| AD 3 Temporal Ctx | 14.2 | Control 4 Occipital | 16.6 |
| | | Ctx | |
| AD 4 Temporal Ctx | 23.8 | Control (Path) 1 | 57.4 |
| | | Occipital Ctx | |
| AD 5 Inf Temporal | 100.0 | Control (Path) 2 | 12.9 |
| Ctx | | Occipital Ctx | |
| AD 5 SupTemporal | 69.7 | Control (Path) 3 | 14.6 |
| Ctx | | Occipital Ctx | |
| AD 6 Inf Temporal | 73.2 | Control (Path) 4 | 12.5 |
| Ctx | | Occipital Ctx | |
| AD 6 Sup Temporal | 57.8 | Control 1 Parietal | 11.7 |
| Ctx | | Ctx | |
| Control 1 Temporal | 17.3 | Control 2 Parietal | 50.0 |
| Ctx | | Ctx | |
| Control 2 Temporal | 47.6 | Control 3 Parietal | 39.0 |
| Ctx | | Ctx | |
| Control 3 Temporal | 32.5 | Control (Path) 1 | 41.8 |
| Ctx | | Parietal Ctx | |
| Control 4 Temporal | 20.9 | Control (Path) 2 | 18.4 |
| Ctx | | Parietal Ctx | |
| Control (Path) 1 | 35.6 | Control (Path) 3 | 16.0 |
| Temporal Ctx | | Parietal Ctx | |
| Control (Path) 2 | 28.3 | Control (Path) 4 | 20.4 |
| Temporal Ctx | | Parietal Ctx | |

TABLE DJC
General_screening_panel_v1.4

| | Rel. Exp. | | Rel. Exp. |
|---|---|---|---|
| | (%) Ag3647, | | (%) Ag3647, |
| | Run | | Run |
| Tissue Name | 218342103 | Tissue Name | 218342103 |
| Adipose | 37.9 | Renal ca. TK-10 | 23.3 |
| Melanoma* | 15.7 | Bladder | 24.1 |
| Hs688(A).T | | | |
| Melanoma* | 20.2 | Gastric ca. (liver met.) | 100.0 |
| Hs688(B).T | | NCI-N87 | |
| Melanoma* M14 | 11.3 | Gastric ca. KATO III | 25.2 |
| Melanoma* | 8.5 | Colon ca. SW-948 | 11.2 |
| LOXIMVI | | | |
| Melanoma* SK- | 13.0 | Colon ca. SW480 | 38.7 |
| MEL-5 | | | |
| Squamous cell | 35.6 | Colon ca.* (SW480 | 11.7 |
| carcinoma SCC-4 | | met) SW620 | |
| Testis Pool | 6.4 | Colon ca. HT29 | 17.2 |
| Prostate ca.* (bone | 11.7 | Colon ca. HCT-116 | 24.1 |
| met) PC-3 | | | |
| Prostate Pool | 12.7 | Colon ca. CaCo-2 | 78.5 |
| Placenta | 31.6 | Colon cancer tissue | 37.1 |
| Uterus Pool | 4.5 | Colon ca. SW1116 | 8.0 |
| Ovarian ca. | 21.2 | Colon ca. Colo-205 | 4.6 |
| OVCAR-3 | | | |
| Ovarian ca. SK- | 22.2 | Colon ca. SW-48 | 9.3 |
| OV-3 | | | |
| Ovarian ca. | 22.2 | Colon Pool | 13.5 |
| OVCAR-4 | | | |
| Ovarian ca. | 32.5 | Small Intestine Pool | 13.0 |
| OVCAR-5 | | | |
| Ovarian ca. | 32.8 | Stomach Pool | 11.7 |
| IGROV-1 | | | |
| Ovarian ca. | 14.3 | Bone Marrow Pool | 5.7 |
| OVCAR-8 | | | |
| Ovary | 7.9 | Fetal Heart | 5.0 |
| Breast ca. MCF-7 | 50.0 | Heart Pool | 6.4 |
| Breast ca. MDA- | 25.7 | Lymph Node Pool | 11.2 |
| MB-231 | | | |
| Breast ca. BT 549 | 61.1 | Fetal Skeletal Muscle | 4.4 |
| Breast ca. T47D | 87.7 | Skeletal Muscle Pool | 11.6 |
| Breast ca. MDA-N | 7.4 | Spleen Pool | 6.0 |
| Breast Pool | 10.6 | Thymus Pool | 8.3 |
| Trachea | 24.5 | CNS cancer | 14.9 |
| | | (glio/astro) U87-MG | |
| Lung | 3.5 | CNS cancer | 16.6 |
| | | (glio/astro) U-118-MG | |
| Fetal Lung | 95.3 | CNS cancer | 15.7 |
| | | (neuro; met) SK-N-AS | |
| Lung ca. NCI-N417 | 3.9 | CNS cancer (astro) | 11.3 |
| | | SF-539 | |
| Lung ca. LX-1 | 16.4 | CNS cancer (astro) | 34.4 |
| | | SNB-75 | |
| Lung ca. NCI-H146 | 6.0 | CNS cancer (glio) | 48.0 |
| | | SNB-19 | |
| Lung ca. SHP-77 | 23.3 | CNS cancer (glio) | 45.4 |
| | | SF-295 | |
| Lung ca. A549 | 25.0 | Brain (Amygdala) | 12.6 |
| | | Pool | |
| Lung ca. NCI-H526 | 6.4 | Brain (cerebellum) | 37.1 |
| Lung ca. NCI-H23 | 10.2 | Brain (fetal) | 13.6 |
| Lung ca. NCI-H460 | 8.7 | Brain (Hippocampus) | 15.3 |
| | | Pool | |
| Lung ca. HOP-62 | 11.3 | Cerebral Cortex Pool | 15.6 |
| Lung ca. NCI-H522 | 16.8 | Brain (Substantia | 27.0 |
| | | nigra) Pool | |
| Liver | 2.5 | Brain (Thalamus) Pool | 17.0 |
| Fetal Liver | 6.6 | Brain (whole) | 8.9 |
| Liver ca. HepG2 | 16.3 | Spinal Cord Pool | 19.9 |
| Kidney Pool | 24.0 | Adrenal Gland | 9.9 |
| Fetal Kidney | 8.0 | Pituitary gland Pool | 6.3 |
| Renal ca. 786-0 | 11.4 | Salivary Gland | 7.2 |
| Renal ca. A498 | 21.2 | Thyroid (female) | 12.1 |
| Renal ca. ACHN | 10.2 | Pancreatic ca. | 35.1 |
| | | CAPAN2 | |
| Renal ca. UO-31 | 18.3 | Pancreas Pool | 23.3 |

TABLE DJD
Panel 4.1D

| | Rel. | Rel. | | Rel. | Rel. |
|---|---|---|---|---|---|
| | Exp. (%) | Exp. (%) | | Exp. (%) | Exp. (%) |
| | Ag3647, | Ag3647, | | Ag3647, | Ag3647, |
| | Run | Run | | Run | Run |
| Tissue Name | 169975750 | 197444046 | Tissue Name | 169975750 | 197444046 |
| Secondary Th1 act | 7.7 | 7.1 | HUVEC IL-1beta | 20.4 | 22.1 |
| Secondary Th2 act | 10.7 | 8.7 | HUVEC IFN | 21.5 | 21.6 |
| | | | gamma | | |
| Secondary Tr1 act | 9.1 | 7.9 | HUVEC TNF | 39.2 | 26.2 |
| | | | alpha + IFN | | |
| | | | gamma | | |
| Secondary Th1 rest | 4.2 | 2.7 | HUVEC TNF | 20.9 | 16.6 |
| | | | alpha + IL4 | | |
| Secondary Th2 rest | 6.0 | 6.2 | HUVEC IL-11 | 8.5 | 8.8 |
| Secondary Tr1 rest | 5.0 | 4.0 | Lung | 29.9 | 31.0 |
| | | | Microvascular EC | | |
| | | | none | | |
| Primary Th1 act | 8.1 | 6.3 | Lung | 37.4 | 24.1 |
| | | | Microvascular EC | | |
| | | | TNF alpha + IL- | | |
| | | | 1beta | | |
| Primary Th2 act | 9.0 | 9.5 | Microvascular | 17.9 | 12.4 |
| | | | Dermal EC none | | |
| Primary Tr1 act | 10.1 | 10.7 | Microsvasular | 20.0 | 15.6 |
| | | | Dermal EC | | |
| | | | TNF alpha + IL- | | |
| | | | 1beta | | |
| Primary Th1 rest | 6.0 | 2.3 | Bronchial | 41.5 | 34.6 |
| | | | epithelium | | |
| | | | TNF alpha + | | |
| | | | IL-1beta | | |
| Primary Th2 rest | 4.3 | 1.1 | Small airway | 37.4 | 33.2 |
| | | | epithelium none | | |
| Primary Tr1 rest | 7.4 | 5.4 | Small airway | 58.2 | 43.8 |
| | | | epithelium | | |
| | | | TNF alpha + IL- | | |
| | | | 1beta | | |
| CD45RA CD4 | 4.2 | 4.2 | Coronery artery | 6.3 | 8.0 |
| lymphocyte act | | | SMC rest | | |
| CD45RO CD4 | 7.1 | 5.7 | Coronery artery | 9.7 | 7.8 |
| lymphocyte act | | | SMC TNF alpha + | | |
| | | | IL-1beta | | |
| CD8 lymphocyte | 6.2 | 4.5 | Astrocytes rest | 11.7 | 4.2 |
| act | | | | | |
| Secondary CD8 | 9.7 | 8.5 | Astrocytes | 11.1 | 7.2 |
| lymphocyte rest | | | TNF alpha + IL- | | |
| | | | 1beta | | |
| Secondary CD8 | 6.5 | 3.7 | KU-812 | 10.0 | 7.9 |
| lymphocyte act | | | (Basophil) rest | | |
| CD4 lymphocyte | 1.8 | 1.7 | KU-812 | 14.4 | 13.3 |
| none | | | (Basophil) | | |
| | | | PMA/ionomycin | | |
| 2ry | 3.1 | 2.9 | CCD1106 | 42.0 | 46.7 |
| Th1/Th2/Tr1_anti- | | | (Keratinocytes) | | |
| CD95 CH11 | | | none | | |
| LAK cells rest | 9.9 | 5.9 | CCD1106 | 100.0 | 100.0 |
| | | | (Keratinocytes) | | |
| | | | TNF alpha + IL- | | |
| | | | 1beta | | |
| LAK cells IL-2 | 5.3 | 4.5 | Liver cirrhosis | 14.7 | 16.2 |
| LAK cells IL- | 9.2 | 3.2 | NCI-H292 none | 17.1 | 16.2 |
| 2 + IL-12 | | | | | |
| LAK cells IL- | 6.1 | 3.8 | NCI-H292 IL-4 | 19.1 | 25.0 |
| 2 + IFN gamma | | | | | |
| LAK cells IL-2 + | 11.3 | 5.5 | NCI-H292 IL-9 | 25.7 | 23.8 |
| IL-18 | | | | | |
| LAK cells | 20.9 | 16.2 | NCI-H292 IL-13 | 20.6 | 18.8 |
| PMA/ionomycin | | | | | |
| NK Cells IL-2 rest | 8.2 | 12.1 | NCI-H292 IFN | 42.6 | 28.3 |
| | | | gamma | | |
| Two Way MLR 3 | 9.2 | 11.4 | HPAEC none | 13.6 | 11.7 |
| day | | | | | |
| Two Way MLR 5 | 8.4 | 4.9 | HPAEC TNF | 20.3 | 22.1 |
| day | | | alpha + IL-1beta | | |
| Two Way MLR 7 | 8.7 | 5.8 | Lung fibroblast | 16.4 | 17.3 |
| day | | | none | | |
| PBMC rest | 3.0 | 2.2 | Lung fibroblast | 11.6 | 13.6 |
| | | | TNF alpha + IL- | | |
| | | | 1beta | | |
| PBMC PWM | 11.0 | 7.6 | Lung fibroblast | 14.6 | 28.3 |
| | | | IL-4 | | |
| PBMC PHA-L | 9.1 | 4.6 | Lung fibroblast | 16.3 | 23.8 |
| | | | IL-9 | | |
| Ramos (B cell) | 5.2 | 3.6 | Lung fibroblast | 14.7 | 12.9 |
| none | | | IL-13 | | |
| Ramos (B cell) | 5.1 | 4.9 | Lung fibroblast | 22.7 | 23.8 |
| ionomycin | | | IFN gamma | | |
| B lymphocytes | 4.1 | 3.8 | Dermal fibroblast | 12.0 | 13.6 |
| PWM | | | CCD1070 rest | | |
| B lymphocytes | 8.4 | 4.9 | Dermal fibroblast | 10.9 | 14.7 |
| CD40L and IL-4 | | | CCD1070 TNF | | |
| | | | alpha | | |
| EOL-1 dbcAMP | 7.3 | 5.5 | Dermal fibroblast | 10.0 | 4.4 |
| | | | CCD1070 IL-1beta | | |
| EOL-1 dbcAMP | 8.4 | 7.8 | Dermal fibroblast | 9.2 | 7.7 |
| PMA/ionomycin | | | IFN gamma | | |
| Dendritic cells | 7.5 | 8.0 | Dermal fibroblast | 9.7 | 7.5 |
| none | | | IL-4 | | |
| Dendritic cells LPS | 5.7 | 4.6 | Dermal | 10.1 | 8.5 |
| | | | Fibroblasts rest | | |
| Dendritic cells | 7.7 | 6.9 | Neutrophils | 11.4 | 5.8 |
| anti-CD40 | | | TNFa + LPS | | |
| Monocytes rest | 8.5 | 7.5 | Neutrophils rest | 3.0 | 3.0 |
| Monocytes LPS | 24.8 | 25.9 | Colon | 13.0 | 9.6 |
| Macrophages rest | 8.4 | 5.6 | Lung | 16.4 | 8.4 |
| Macrophages LPS | 12.2 | 5.9 | Thymus | 10.9 | 8.8 |
| HUVEC none | 8.7 | 12.8 | Kidney | 6.3 | 4.7 |
| HUVEC starved | 11.8 | 11.7 | | | |

CNS_neurodegeneration_v1.0 Summary: Ag3647 This panel confirms the expression of this gene at moderate levels in the brains of an independent group of individuals. However, no differential expression of this gene was detected between Alzheimer's diseased postmortem brains and those of non-demented controls in this experiment. Please see Panel 1.4 for a discussion of the potential utility of this gene in treatment of central nervous system disorders.

General_screening_panel_v1.4 Summary: Ag3647 Expression of the CG88613-01 gene is highest in a gastric cancer cell line (CT=28). Expression of this gene appears to be upregulated in a number of cancer cell lines when compared to normal tissues. Specifically, CG88613-01 gene expression is somewhat higher in breast and ovarian cancers when compared to their respective normal tissues. Thus, therapeutic modulation of the activity of this gene or its protein product, using small molecule drugs, antibodies or protein therapeutics, may be of benefit in the treatment of gastric, breast and ovarian cancer.

In addition, this gene is expressed at moderate levels in all regions of the central nervous system examined, including amygdala, hippocampus, substantia nigra, thalamus, cerebellum, cerebral cortex, and spinal cord. The CG88613-01 gene encodes a protein that is identical to a protein now known in the public domain as inositol 1,4,5-triphosphate 3-kinase C (ref. 1). Inositol 1,4,5-trisphosphate 3-kinase (ITPK) catalyzes the phosphorylation of Ins(1,4,5)P3 to Ins(1,4,5)P4, both of which are modulators of calcium homeostasis. Calcium is one of the most important intracellular messengers in the brain, being essential for neuronal development, synaptic transmission and plasticity, and the regulation of various metabolic pathways (ref. 2). Therefore, this gene may play a role in central nervous system disorders such as Alzheimer's disease, Parkinson's disease, epilepsy, multiple sclerosis, schizophrenia and depression. Furthermore, this gene is also expressed in tissues with metabolic or endocrine function, including pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

### REFERENCES

1. Dewaste V, Pouillon V, Moreau C, Shears S, Takazawa K, Erneux C. Cloning and expression of a cDNA encoding human inositol 1,4,5-trisphosphate 3-kinase C. Biochem J Dec. 1, 2000;352 Pt 2:343-51

2. Mattson M P, Chan S L. Dysregulation of cellular calcium homeostasis in Alzheimer's disease: bad genes and bad habits. J Mol Neurosci October 2001;17(2):205-24

Panel 4.1D Summary: Ag3647 Results from two experiments using the same probe/primer set are in excellent agreement. Expression of the CG88613-01 gene is highest in keratinocytes treated with the inflammatory cytokines TNF-a and IL-1b(CT=29.5). Therefore, modulation of the expression or activity of this protein through the application of small molecule therapeutics may be useful in the treatment of psoriasis and wound healing.

This gene is also expressed at moderate levels in small airway epithelial cells, bronchial epithelium, and lung microvascular endothelial cells. Endothelial cells are known to play important roles in inflammatory responses by altering the expression of surface proteins that are involved in activation and recruitment of effector inflammatory cells (ref. 1). Expression in small airway epithelial cells, bronchial epithelium, lung microvascular endothelial cells suggests that the protein encoded by this transcript may be involved in lung disorders including asthma, allergies, chronic obstructive pulmonary disease, and emphysema. This gene is homologoust o PI-3-kinase which is involved in cell survival and receptor signaling of a number of cells of importance in the immune response in health and disease, including lung pathologies. Therefore, Small molecule antagonists of this gene product may lead to amelioration of symptoms associated with asthma, allergies, chronic obstructive pulmonary disease, and emphysema.

This gene is expressed at low levels in the remainder of the samples on this panel, suggesting that the gene product may play an important role in homeostasis of a number of cell types.

### REFERENCES

1. Siddiqui R A, English D. Phosphatidylinositol 3′-kinase-mediated calcium mobilization regulates chemotaxis in phosphatidic acid-stimulated human neutrophils. Biochim Biophys Acta Jan. 3, 2000;1483(1):161-73

2. Condliffe A M, Cadwallader K A, Walker T R, Rintoul R C, Cowburn A S, Chilvers E R. Phosphoinositide 3-kinase: a critical signalling event in pulmonary cells. Respir Res 2000;1(1):24-9

DK. CG59993-01 and CG59993-02: Synaptotagmin II

Expression of gene CG59993-01 and variant CG59993-02 was assessed using the primer-probe set Ag3645, described in Table DKA. Results of the RTQ-PCR runs are shown in Tables DKB, DKC and DKD.

TABLE DKA

Probe Name Ag3645

| | | | Start | SEQ ID |
|---|---|---|---|---|
| Primers | Sequences | Length | Position | NO: |
| Forward | 5′-gaagaagaccctgaacccatac-3′ | 22 | 1056 | 746 |
| Probe | TET-5′-agctttgagatccccttcgagcagat-3′-TAMRA | 26 | 1093 | 747 |
| Reverse | 5′-tgaccactacctggactttctg-3′ | 22 | 1120 | 748 |

TABLE DKB
CNS_neurodegeneration_v1.0

| | Rel. Exp. | | Rel. Exp. |
|---|---|---|---|
| | (%) Ag3645, | | (%) Ag3645 |
| | Run | | Run |
| Tissue Name | 211019104 | Tissue Name | 211019104 |
| AD 1 Hippo | 0.4 | Control (Path) 3 | 0.2 |
| | | Temporal Ctx | |
| AD 2 Hippo | 0.2 | Control (Path) 4 | 7.2 |
| | | Temporal Ctx | |
| AD 3 Hippo | 0.0 | AD 1 Occipital | 8.7 |
| | | Ctx | |
| AD 4 Hippo | 0.1 | AD 2 Occipital | 0.0 |
| | | Ctx (Missing) | |
| AD 5 hippo | 100.0 | AD 3 Occipital | 0.6 |
| | | Ctx | |
| AD 6 Hippo | 1.5 | AD 4 Occipital | 10.7 |
| | | Ctx | |
| Control 2 Hippo | 0.9 | AD 5 Occipital | 19.2 |
| | | Ctx | |
| Control 4 Hippo | 0.1 | AD 6 Occipital | 57.8 |
| | | Ctx | |
| Control (Path) 3 | 0.0 | Control 1 Occipital | 0.3 |
| Hippo | | Ctx | |
| AD 1 Temporal Ctx | 0.8 | Control 2 Occipital | 81.8 |
| | | Ctx | |
| AD 2 Temporal Ctx | 2.8 | Control 3 Occipital | 11.0 |
| | | Ctx | |
| AD 3 Temporal Ctx | 0.3 | Control 4 Occipital | 0.5 |
| | | Ctx | |
| AD 4 Temporal Ctx | 2.6 | Control (Path) 1 | 29.1 |
| | | Occipital Ctx | |
| AD 5 Inf Temporal | 50.0 | Control (Path) 2 | 8.5 |
| Ctx | | Occipital Ctx | |
| AD 5 SupTemporal | 0.9 | Control (Path) 3 | 0.7 |
| Ctx | | Occipital Ctx | |
| AD 6 Inf Temporal | 2.8 | Control (Path) 4 | 12.8 |
| Ctx | | Occipital Ctx | |
| AD 6 Sup Temporal | 8.2 | Control 1 Parietal | 0.4 |
| Ctx | | Ctx | |
| Control 1 Temporal | 0.1 | Control 2 Parietal | 2.3 |
| Ctx | | Ctx | |
| Control 2 Temporal | 7.2 | Control 3 Parietal | 28.7 |
| Ctx | | Ctx | |
| Control 3 Temporal | 1.5 | Control (Path) 1 | 33.2 |
| Ctx | | Parietal Ctx | |
| Control 4 Temporal | 0.7 | Control (Path) 2 | 21.8 |
| Ctx | | Parietal Ctx | |
| Control (Path) 1 | 4.7 | Control (Path) 3 | 0.5 |
| Temporal Ctx | | Parietal Ctx | |
| Control (Path) 2 | 6.8 | Control (Path) 4 | 68.3 |
| Temporal Ctx | | Parietal Ctx | |

TABLE DKC
General_screening_panel_v1.4

| | Rel. Exp. | | Rel. Exp. |
|---|---|---|---|
| | (%) Ag3645, | | (%) Ag3645, |
| | Run | | Run |
| Tissue Name | 218341901 | Tissue Name | 218341901 |
| Adipose | 0.0 | Renal ca. TK-10 | 0.1 |
| Melanoma* | 0.1 | Bladder | 0.1 |
| Hs688(A).T | | | |
| Melanoma* | 0.1 | Gastric ca. (liver met.) | 0.0 |
| Hs688(B).T | | NCI-N87 | |
| Melanoma* M14 | 0.0 | Gastric ca. KATO III | 0.0 |
| Melanoma* | 0.0 | Colon ca. SW-948 | 0.0 |
| LOXIMVI | | | |
| Melanoma* SK- | 0.4 | Colon ca. SW480 | 0.0 |
| MEL-5 | | | |
| Squamous cell | 0.1 | Colon ca.* (SW480 | 0.0 |
| carcinoma SCC-4 | | met) SW620 | |
| Testis Pool | 1.0 | Colon ca. HT29 | 0.0 |
| Prostate ca.* (bone | 0.0 | Colon ca. HCT-116 | 0.0 |
| met) PC-3 | | | |
| Prostate Pool | 0.1 | Colon ca. CaCo-2 | 0.6 |
| Placenta | 0.0 | Colon cancer tissue | 0.0 |
| Uterus Pool | 0.0 | Colon ca. SW1116 | 0.0 |
| Ovarian ca. | 0.2 | Colon ca. Colo-205 | 0.0 |
| OVCAR-3 | | | |
| Ovarian ca. SK- | 0.0 | Colon ca. SW-48 | 0.2 |
| OV-3 | | | |
| Ovarian ca. | 0.2 | Colon Pool | 0.2 |
| OVCAR-4 | | | |
| Ovarian ca. | 0.1 | Small Intestine Pool | 0.1 |
| OVCAR-5 | | | |
| Ovarian ca. | 0.0 | Stomach Pool | 0.1 |
| IGROV-1 | | | |
| Ovarian ca. | 0.0 | Bone Marrow Pool | 0.0 |
| OVCAR-8 | | | |
| Ovary | 0.7 | Fetal Heart | 0.1 |
| Breast ca. MCF-7 | 0.0 | Heart Pool | 0.0 |
| Breast ca. MDA- | 1.4 | Lymph Node Pool | 1.0 |
| MB-231 | | | |
| Breast ca. BT 549 | 0.2 | Fetal Skeletal Muscle | 0.0 |
| Breast ca. T47D | 0.1 | Skeletal Muscle Pool | 0.0 |
| Breast ca. MDA-N | 0.0 | Spleen Pool | 0.1 |
| Breast Pool | 0.2 | Thymus Pool | 0.5 |
| Trachea | 0.2 | CNS cancer | 0.0 |
| | | (glio/astro) U87-MG | |
| Lung | 0.3 | CNS cancer | 0.0 |
| | | (glio/astro) U-118-MG | |
| Fetal Lung | 0.5 | CNS cancer | 0.2 |
| | | (neuro; met) SK-N-AS | |
| Lung ca. NCI-N417 | 0.5 | CNS cancer (astro) | 0.0 |
| | | SF-539 | |
| Lung ca. LX-1 | 0.0 | CNS cancer (astro) | 0.2 |
| | | SNB-75 | |
| Lung ca. NCI-H146 | 0.1 | CNS cancer (glio) | 0.0 |
| | | SNB-19 | |
| Lung ca. SHP-77 | 0.3 | CNS cancer (glio) | 0.1 |
| | | SF-295 | |
| Lung ca. A549 | 0.0 | Brain (Amygdala) | 5.9 |
| | | Pool | |
| Lung ca. NCI-H526 | 0.0 | Brain (cerebellum) | 100.0 |
| Lung ca. NCI-H23 | 0.2 | Brain (fetal) | 0.9 |
| Lung ca. NCI-H460 | 0.0 | Brain (Hippocampus) | 2.1 |
| | | Pool | |
| Lung ca. HOP-62 | 0.0 | Cerebral Cortex Pool | 19.3 |
| Lung ca. NCI-H522 | 0.0 | Brain (Substantia | 19.5 |
| | | nigra) Pool | |
| Liver | 0.0 | Brain (Thalamus) Pool | 14.1 |
| Fetal Liver | 0.0 | Brain (whole) | 10.0 |
| Liver ca. HepG2 | 0.0 | Spinal Cord Pool | 15.9 |
| Kidney Pool | 0.1 | Adrenal Gland | 0.9 |
| Fetal Kidney | 0.6 | Pituitary gland Pool | 0.0 |
| Renal ca. 786-0 | 0.0 | Salivary Gland | 0.0 |
| Renal ca. A498 | 0.0 | Thyroid (female) | 0.1 |
| Renal ca. ACHN | 0.0 | Pancreatic ca. | 0.0 |
| | | CAPAN2 | |
| Renal ca. UO-31 | 0.0 | Pancreas Pool | 0.8 |

TABLE DKD
Panel 4.1D

| | Rel. | | Rel. |
|---|---|---|---|
| | Exp. | | Exp. |
| | (%) | | (%) |
| | Ag3645, | | Ag3645, |
| | Run | | Run |
| Tissue Name | 169975206 | Tissue Name | 169975206 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular | 0.0 |
| | | EC none | |
| Primary Th1 act | 0.0 | Lung Microvascular | 0.0 |
| | | EC TNFalpha + | |
| | | IL-1beta | |
| Primary Th2 act | 0.0 | Microvascular Dermal | 0.0 |
| | | EC none | |
| Primary Tr1 act | 0.0 | Microvasular Dermal | 0.0 |
| | | EC TNFalpha + | |
| | | IL-1beta | |
| Primary Th1 rest | 0.0 | Bronchial epithelium | 0.0 |
| | | TNFalpha + IL1beta | |
| Primary Th2 rest | 0.0 | Small airway | 0.0 |
| | | epithelium none | |
| Primary Tr1 rest | 0.0 | Small airway | 0.0 |
| | | epithelium | |
| | | TNFalpha + IL-beta | |
| CD45RA CD4 | 0.0 | Coronery artery | 0.0 |
| lymphocyte act | | SMC rest | |
| CD45RO CD4 | 0.0 | Coronery artery | 0.0 |
| lymphocyte act | | SMC TNFalpha + | |
| | | IL-1beta | |
| CD8 lymphocyte act | 0.0 | Astrocytes rest | 0.0 |
| Secondary CD8 | 0.0 | Astrocytes | 100.0 |
| lymphocyte rest | | TNFalpha + | |
| | | IL-1beta | |
| Secondary CD8 | 0.0 | KU-812 (Basophil) | 0.0 |
| lymphocyte act | | rest | |
| CD4 lymphocyte | 0.0 | KU-812 (Basophil) | 0.0 |
| none | | PMA/ionomycin | |
| 2ry Th1/Th2/ | 0.0 | CCD1106 | 0.0 |
| Tr1_anti- | | (Keratinocytes) | |
| CD95 CH11 | | none | |
| LAK cells rest | 0.0 | CCD1106 | 0.0 |
| | | (Keratinocytes) | |
| | | TNFalpha + IL-1beta | |
| LAK cells IL-2 | 0.0 | Liver cirrhosis | 0.0 |
| LAK cells IL-2 + | 0.0 | NCI-H292 none | 15.0 |
| IL-12 | | | |
| LAK cells IL-2 + | 8.3 | NCI-H292 IL-4 | 0.0 |
| IFN gamma | | | |
| LAK cells IL-2 + | 0.0 | NCI-H292 IL-9 | 22.5 |
| IL-18 | | | |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR | 0.0 | HPAEC none | 0.0 |
| 3 day | | | |
| Two Way MLR | 0.0 | HPAEC TNF alpha + | 0.0 |
| 5 day | | IL-1beta | |
| Two Way MLR | 0.0 | Lung fibroblast none | 0.0 |
| 7 day | | | |
| PBMC rest | 0.0 | Lung fibroblast | 4.3 |
| | | TNF alpha + | |
| | | IL-1beta | |
| PBMC PWM | 0.0 | Lung fibroblast IL-4 | 0.0 |
| PBMC PHA-L | 12.9 | Lung fibroblast IL-9 | 0.0 |
| Ramos (B cell) none | 0.0 | Lung fibroblast IL-13 | 36.9 |
| Ramos (B cell) | 0.0 | Lung fibroblast IFN | 0.0 |
| ionomycin | | gamma | |
| B lymphocytes | 0.0 | Dermal fibroblast | 0.0 |
| PMW | | CCD1070 rest | |
| B lymphocytes | 0.0 | Dermal fibroblast | 0.0 |
| CD40L and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 48.6 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts | 0.0 |
| | | rest | |
| Dendritic cells anti- | 20.9 | Neutrophils TNFa + | 0.0 |
| CD40 | | LPS | |
| Monocytes rest | 0.0 | Neutrophils rest | 0.0 |
| Monocytes LPS | 0.0 | Colon | 25.7 |
| Macrophages rest | 0.0 | Lung | 28.7 |
| Macrophages LPS | 0.0 | Thymus | 43.2 |
| HUVEC none | 0.0 | Kidney | 25.9 |
| HUVEC starved | 0.0 | | |

CNS_nturodegeneration_v1.0 Summary: Ag3645 While no association between the expression of the CG59993-01 gene and the presence of Alzheimer's disease is detected in this panel, these results confirm the expression of this gene in areas that degenerate in Alzheimer's disease, including the cortex and hippocampus. Synaptotagmin expression is altered in the brain of Alzheimer's patients, possibly explaining impaired synaptogenesis and/or synaptosomal loss secondary to neuronal loss observed in the neurodegenerative disorder. It may also represent, reflect or account for the impaired neuronal transmission in Alzheimer's disease (AD), caused by deterioration of the exocytic machinery. Since the this gene is a homolog of synaptotagmin, agents that potentiate the expression or function of the protein encoded by the this gene may be useful in the treatment of Alzheimer's disease.

General_screening_panel_v1.4 Summary: Ag3645 The CG59993-01 gene is a homolog of synaptotagmin, and shows high to moderate expression across all brain regions with highest expression in the cerebellum (CT=26.4) Synaptotagmin is a presynaptic protein involved in synaptic vesicle release, making this an ideal drug target for diseases such as epilepsy, in which reduction of neurotransmission is beneficial. Selective inhibition of this gene or its protein product may therefore be useful in the treatment of seizure disorders. Furthermore, selective inhibition of neural transmission through antagonism of the protein encoded by this gene may show therapeutic benefit in psychiatric diseases where it is believed that inappropriate neural connections have been established, such as schizophrenia and bipolar disorder. In addition, antibodies against synaptotagmin may cause Lambert-Eaton myasthenic syndrome. Therefore, peptide fragments of the protein encoded by this gene may serve to block the action of these antibodies and treat Lambert-Eaton myasthenic syndrome.

Panel 4.1D Summary: Ag3645 Expression of the CG59993-01 gene is restricted to a sample derived from astrocytes treated with TNF-alpha and IL-1 beta (CT=33.9). This expression in samples related to the central nervous system is consistent with results of the previous panels and suggests that modulation of this protein could be beneficial in the treatment of CNS disease-associated inflammation or neurodegeneration, including mutliple sclerosis.

DL. CG59991-01: Ooplasm Specific Protein

Expression of gene CG59991-01 was assessed using the primer-probe set Ag3644, described in Table DLA. Results of the RTQ-PCR runs are shown in Tables DLB and DLC.

TABLE DLA
Probe Name Ag3644

| | | | Start | SEQ ID |
|---|---|---|---|---|
| Primers | Sequences | Length | Position | NO: |
| Forward | 5′-gggagtaatgcctctcagtga-3′ | 21 | 2294 | 749 |
| Probe | TET-5′-cttgagagtctcccagtgcgccct-3′-TAMRA | 24 | 2318 | 750 |
| Reverse | 5′-atgccacagtcctccagtatc-3′ | 21 | 2351 | 751 |

TABLE DLB
General_screening_panel_v1.4

| | Rel. | | Rel. |
|---|---|---|---|
| | Exp. | | Exp. |
| | (%) | | (%) |
| | Ag3644, | | Ag3644, |
| | Run | | Run |
| Tissue Name | 218306573 | Tissue Name | 218306573 |
| Adipose | 0.0 | Renal ca. TK-10 | 0.0 |
| Melanoma* | 0.0 | Bladder | 0.0 |
| Hs688(A).T | | | |
| Melanoma* | 0.0 | Gastric ca. (liver met.) | 0.0 |
| Hs688(B).T | | NCI-N87 | |
| Melanoma* M14 | 0.0 | Gastric ca. KATO III | 0.1 |
| Melanoma* | 0.0 | Colon ca. SW-948 | 0.0 |
| LOXIMVI | | | |
| Melanoma* SK- | 0.0 | Colon ca. SW480 | 0.0 |
| MEL-5 | | | |
| Squamous cell | 0.0 | Colon ca.* (SW480 | 0.0 |
| carcinoma SCC-4 | | met) SW620 | |
| Testis Pool | 0.5 | Colon ca. HT29 | 0.0 |
| Prostate ca.* (bone | 0.0 | Colon ca. HCT-116 | 0.1 |
| met) PC-3 | | | |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. | 0.0 | Colon ca. Colo-205 | 0.0 |
| OVCAR-3 | | | |
| Ovarian ca. | 0.0 | Colon ca. SW-48 | 0.0 |
| SK-OV-3 | | | |
| Ovarian ca. | 0.6 | Colon Pool | 0.0 |
| OVCAR-4 | | | |
| Ovarian ca. | 0.0 | Small Intestine Pool | 0.0 |
| OVCAR-5 | | | |
| Ovarian ca. | 0.0 | Stomach Pool | 0.0 |
| IGROV-1 | | | |
| Ovarian ca. | 0.0 | Bone Marrow Pool | 0.0 |
| OVCAR-8 | | | |
| Ovary | 0.0 | Fetal Heart | 0.0 |
| Breast ca. MCF-7 | 0.0 | Heart Pool | 0.0 |
| Breast ca. MDA- | 0.0 | Lymph Node Pool | 0.0 |
| MB-231 | | | |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 0.0 | Thymus Pool | 0.0 |
| Trachea | 0.0 | CNS cancer | 0.0 |
| | | (glio/astro) | |
| | | U87-MG | |
| Lung | 0.0 | CNS cancer | 0.0 |
| | | (glio/astro) | |
| | | U-118-MG | |
| Fetal Lung | 0.0 | CNS cancer | 0.0 |
| | | (neuro; met) | |
| | | SK-N-AS | |
| Lung ca. NCI-N417 | 0.0 | CNS cancer | 0.0 |
| | | (astro) | |
| | | SF-539 | |
| Lung ca. LX-1 | 0.0 | CNS cancer (astro) | 0.0 |
| | | SNB-75 | |
| Lung ca. NCI-H146 | 0.0 | CNS cancer (glio) | 0.0 |
| | | SNB-19 | |
| Lung ca. SHP-77 | 100.0 | CNS cancer (glio) | 0.3 |
| | | SF-295 | |
| Lung ca. A549 | 0.0 | Brain (Amygdala) | 0.0 |
| | | Pool | |
| Lung ca. NCI-H526 | 0.0 | Brain (cerebellum) | 0.0 |
| Lung ca. NCI-H23 | 0.0 | Brain (fetal) | 0.0 |
| Lung ca. NCI-H460 | 0.0 | Brain (Hippocampus) | 0.0 |
| | | Pool | |
| Lung ca. HOP-62 | 0.0 | Cerebral Cortex Pool | 0.0 |
| Lung ca. NCI-H522 | 0.0 | Brain | 0.0 |
| | | (Substantia nigra) | |
| | | Pool | |
| Liver | 0.0 | Brain (Thalamus) Pool | 0.0 |
| Fetal Liver | 0.0 | Brain (whole) | 0.0 |
| Liver ca. HepG2 | 0.0 | Spinal Cord Pool | 0.0 |
| Kidney Pool | 0.0 | Adrenal Gland | 0.0 |
| Fetal Kidney | 0.0 | Pituitary gland Pool | 0.0 |
| Renal ca. 786-0 | 0.0 | Salivary Gland | 0.0 |
| Renal ca. A498 | 0.0 | Thyroid (female) | 0.1 |
| Renal ca. ACHN | 0.0 | Pancreatic ca. | 0.0 |
| | | CAPAN2 | |
| Renal ca. UO-31 | 0.0 | Pancreas Pool | 0.1 |

TABLE DLC
Panel 4.1D

| | Rel. | | Rel. |
|---|---|---|---|
| | Exp. | | Exp. |
| | (%) | | (%) |
| | Ag3644, | | Ag3644, |
| | Run | | Run |
| Tissue Name | 169975188 | Tissue Name | 169975188 |
| Secondary Th1 act | 0.0 | HUVEC IL-beta | 0.0 |
| Secondary Th2 act | 0.0 | HUVEC IFN gamma | 0.0 |
| Secondary Tr1 act | 0.0 | HUVEC TNF alpha + | 0.0 |
| | | IFN gamma | |
| Secondary Th1 rest | 0.0 | HUVEC TNF alpha + | 0.0 |
| | | IL4 | |
| Secondary Th2 rest | 0.0 | HUVEC IL-11 | 0.0 |
| Secondary Tr1 rest | 0.0 | Lung Microvascular | 0.0 |
| | | EC none | |
| Primary Th1 act | 0.0 | Lung Microvascular | 0.0 |
| | | EC TNFalpha + | |
| | | IL-1beta | |
| Primary Th2 act | 0.0 | Microvascular Dermal | 0.0 |
| | | EC none | |
| Primary Tr1 act | 0.0 | Microvasular Dermal | 0.0 |
| | | EC TNFalpha + | |
| | | IL-1beta | |
| Primary Tr1 act | 0.0 | Microvascular Dermal | 0.0 |
| | | EC TNFalpha + | |
| | | IL-1beta | |
| Primary Th1 rest | 0.0 | Bronchial epithelium | 0.0 |
| | | TNFalpha + IL1beta | |
| Primary Th2 rest | 0.0 | Small airway | 0.0 |
| | | epithelium none | |
| Primary Tr1 rest | 0.0 | Small airway | 0.0 |
| | | epithelium | |
| | | TNFalpha + IL-beta | |
| CD45RA CD4 | 0.0 | Coronery artery | 0.0 |
| lymphocyte act | | SMC rest | |
| CD45RO CD4 | 0.0 | Coronery artery | 0.0 |
| lymphocyte act | | SMC TNFalpha + | |
| | | IL-1beta | |
| CD8 lymphocyte act | 0.0 | Astrocytes rest | 0.0 |
| Secondary CD8 | 0.0 | Astrocytes | 0.0 |
| lymphocyte rest | | TNFalpha + | |
| | | IL-1beta | |
| Secondary CD8 | 0.0 | KU-812 (Basophil) | 48.3 |
| lynphocyte act | | rest | |
| CD4 lymphocyte | 0.0 | KU-812 (Basophil) | 100.0 |
| none | | PMA/ionomycin | |
| 2ry Th1/Th2/ | 0.0 | CCD1106 | 0.0 |
| Tr1_anti- | | (Keratinocytes) | |
| CD95 CH11 | | none | |
| LAK cells rest | 0.0 | CCD1106 | 0.0 |
| | | (Keratinocytes) | |
| | | TNFalpha + IL-1beta | |
| LAK cells IL-2 | 0.0 | Liver cirrhosis | 0.0 |
| LAK cells IL-2 + | 0.0 | NCI-H292 none | 0.0 |
| IL-12 | | | |
| LAK cells IL-2 + | 0.0 | NCI-H292 IL-4 | 0.0 |
| IFN gamma | | | |
| LAK cells IL-2 + | 0.0 | NCI-H292 IL-9 | 0.0 |
| IL-18 | | | |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR | 0.0 | HPAEC none | 0.0 |
| 3 day | | | |
| Two Way MLR | 0.0 | HPAEC TNF alpha + | 0.0 |
| 5 day | | IL-1beta | |
| Two Way MLR | 0.0 | Lung fibroblast none | 0.0 |
| 7 day | | | |
| PBMC rest | 0.0 | Lung fibroblast | 0.0 |
| | | TNF alpha + | |
| | | IL-1beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes | 0.0 | Dermal fibroblast | 0.0 |
| PMW | | CCD1070 rest | |
| B lymphocytes | 0.0 | Dermal fibroblast | 0.0 |
| CD40L and IL-4 | | CCD1070 TNF alpha | |
| EOL-1 dbcAMP | 0.0 | Dermal fibroblast | 0.0 |
| | | CCD1070 IL-1beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts | 0.0 |
| | | rest | |
| Dendritic cells anti- | 0.0 | Neutrophils TNFa + | 0.0 |
| CD40 | | LPS | |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_{—}1.0 Summary: Ag3644 Expression of the CG59991-01 gene is low/undetectable in all samples on this panel (CTs>35). (Data not shown).

General_screening_panel_v1.4 Summary: Ag3644 Expression of the CG59991-01 gene is restricted to a sample derived from a lung cancer cell line (CT=27.2). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

Panel 4.1D Summary: Ag3644 Expression of the CG59991-01 gene is restricted to samples derived from the basophil cell line KU-812 (CTs=32). Thus, expression of this gene could be used as a marker of this cell type. Basophils release histamines and other biological modifiers in repose to allergens and play an important role in the pathology of asthma and hypersensitivity reactions. Therefore, the specific pattern of expression of this gene suggests that therapeutic modulation of the expression or function of the protein encoded by this gene may block or inhibit inflammation or tissue damage due to basophil activation in response to asthma, allergies, hypersensitivity reactions, psoriasis, and viral infections.

DM. CG59987-01 and CG59987-02: Rhophilin

Expression of gene CG59987-01 and full length clone CG59987-02 was assessed using the primer-probe set Ag3643, described in Table DMA. Results of the RTQ-PCR runs are shown in Tables DMB and DMC. Please note that CG59987-02 represents a full-length physical clone of the CG59987-01 gene, validating the prediction of the gene sequence.

Table DMA. Probe Name Ag3643

TABLE DMA
Probe Name Ag3643

| | | | Start | SEQ ID |
|---|---|---|---|---|
| Primers | Sequences | Length | Position | NO: |
| Forward | 5′-tactttcggaagggctgtaatc-3′ | 22 | 103 | 752 |
| Probe | TET-5′-cttgcacaaaccggccggagtaaatt-3′-TAMRA | 26 | 127 | 753 |
| Reverse | 5′-tgattcaaagcagctctttgat-3′ | 22 | 158 | 754 |

TABLE DMB
General_screening_panel_v1.4

| | Rel. | | Rel. |
|---|---|---|---|
| | Exp. | | Exp. |
| | (%) | | (%) |
| | Ag3643, | | Ag3643, |
| | Run | | Run |
| Tissue Name | 218306426 | Tissue Name | 218306426 |
| Adipose | 1.4 | Renal ca. TK-10 | 17.2 |
| Melanoma* | 0.5 | Bladder | 9.1 |
| Hs688(A).T | | | |
| Melanoma* | 0.6 | Gastric ca. (liver met.) | 46.7 |
| Hs688(B).T | | NCI-N87 | |
| Melanoma* M14 | 6.3 | Gastric ca. KATO III | 39.2 |
| Melanoma* | 1.7 | Colon ca. SW-948 | 16.6 |
| LOXIMVI | | | |
| Melanoma* SK- | 8.7 | Colon ca. SW480 | 7.0 |
| MEL-5 | | | |
| Squamous cell | 5.4 | Colon ca.* (SW480 | 0.4 |
| carcinoma SCC-4 | | met) SW620 | |
| Testis Pool | 0.5 | Colon ca. HT29 | 24.8 |
| Prostate ca.* (bone | 7.7 | Colon ca. HCT-116 | 26.8 |
| met) PC-3 | | | |
| Prostate Pool | 6.6 | Colon ca. CaCo-2 | 34.2 |
| Placenta | 3.3 | Colon cancer tissue | 13.0 |
| Uterus Pool | 0.4 | Colon ca. SW1116 | 4.5 |
| Ovarian ca. | 44.1 | Colon ca. Colo-205 | 5.5 |
| OVCAR-3 | | | |
| Ovarian ca. | 36.9 | Colon ca. SW-48 | 6.5 |
| SK-OV-3 | | | |
| Ovarian ca. | 58.6 | Colon Pool | 0.9 |
| OVCAR-4 | | | |
| Ovarian ca. | 50.7 | Small Intestine Pool | 1.9 |
| OVCAR-5 | | | |
| Ovarian ca. | 20.3 | Stomach Pool | 2.3 |
| IGROV-1 | | | |
| Ovarian ca. | 7.9 | Bone Marrow Pool | 0.3 |
| OVCAR-8 | | | |
| Ovary | 1.6 | Fetal Heart | 0.7 |
| Breast ca. MCF-7 | 17.4 | Heart Pool | 0.4 |
| Breast ca. MDA- | 13.7 | Lymph Node Pool | 1.0 |
| MB-231 | | | |
| Breast ca. BT 549 | 8.2 | Fetal Skeletal Muscle | 0.1 |
| Breast ca. T47D | 100.0 | Skeletal Muscle Pool | 0.2 |
| Breast ca. MDA-N | 4.7 | Spleen Pool | 0.2 |
| Breast Pool | 1.9 | Thymus Pool | 1.5 |
| Trachea | 8.4 | CNS cancer | 1.0 |
| | | (glio/astro) | |
| | | U87-MG | |
| Lung | 0.3 | CNS cancer | 1.3 |
| | | (glio/astro) | |
| | | U-118-MG | |
| Fetal Lung | 6.9 | CNS cancer | 5.2 |
| | | (neuro; met) | |
| | | SK-N-AS | |
| Lung ca. NCI-N417 | 0.9 | CNS cancer | 2.5 |
| | | (astro) | |
| | | SF-539 | |
| Lung ca. LX-1 | 0.2 | CNS cancer (astro) | 6.0 |
| | | SNB-75 | |
| Lung ca. NCI-H146 | 1.9 | CNS cancer (glio) | 21.6 |
| | | SNB-19 | |
| Lung ca. SHP-77 | 0.0 | CNS cancer (glio) | 14.7 |
| | | SF-295 | |
| Lung ca. A549 | 54.3 | Brain (Amygdala) | 1.0 |
| | | Pool | |
| Lung ca. NCI-H526 | 3.7 | Brain (cerebellum) | 5.2 |
| Lung ca. NCI-H23 | 3.2 | Brain (fetal) | 0.7 |
| Lung ca. NCI-H460 | 1.2 | Brain (Hippocampus) | 1.9 |
| | | Pool | |
| Lung ca. HOP-62 | 6.5 | Cerebral Cortex Pool | 2.3 |
| Lung ca. NCI-H522 | 3.7 | Brain | 2.1 |
| | | (Substantia nigra) | |
| | | Pool | |
| Liver | 1.5 | Brain (Thalamus) Pool | 2.7 |
| Fetal Liver | 5.6 | Brain (whole) | 3.5 |
| Liver ca. HepG2 | 7.0 | Spinal Cord Pool | 3.6 |
| Kidney Pool | 1.4 | Adrenal Gland | 0.2 |
| Fetal Kidney | 3.4 | Pituitary gland Pool | 1.3 |
| Renal ca. 786-0 | 12.2 | Salivary Gland | 6.7 |
| Renal ca. A498 | 8.8 | Thyroid (female) | 1.2 |
| Renal ca. ACHN | 9.1 | Pancreatic ca. | 21.3 |
| | | CAPAN2 | |
| Renal ca. UO-31 | 9.9 | Pancreas Pool | 12.2 |

TABLE CMD
Panel 4.1D

| | Rel. | | Rel. |
|---|---|---|---|
| | Exp. | | Exp. |
| | (%) | | (%) |
| | Ag3643, | | Ag3643, |
| | Run | | Run |
| Tissue Name | 169975145 | Tissue Name | 169975145 |
| Secondary Th1 act | 3.8 | HUVEC IL-1beta | 6.9 |
| Secondary Th2 act | 1.1 | HUVEC IFN gamma | 4.4 |
| Secondary Tr1 act | 0.9 | HUVEC TNF alpha + | 2.6 |
| | | IFN gamma | |
| Secondary Th1 rest | 0.5 | HUVEC TNF alpha + | 4.0 |
| | | IL4 | |
| Secondary Th2 rest | 0.9 | HUVEC IL-11 | 2.5 |
| Secondary Tr1 rest | 0.6 | Lung Microvascular | 4.3 |
| | | EC none | |
| Primary Th1 act | 1.8 | Lung Microvascular | 1.5 |
| | | EC TNFalpha + | |
| | | IL-1beta | |
| Primary Th2 act | 5.0 | Microvascular Dermal | 6.7 |
| | | EC none | |
| Primary Tr1 act | 3.4 | Microvascular Dermal | 7.7 |
| | | EC TNFalpha + | |
| | | IL-1beta | |
| Primary Th1 rest | 1.0 | Bronchial epithelium | 17.7 |
| | | TNFalpha + IL1beta | |
| Primary Th2 rest | 0.8 | Small airway | 13.7 |
| | | epithelium none | |
| Primary Tr1 rest | 0.9 | Small airway | 23.3 |
| | | epithelium | |
| | | TNFalpha + IL-beta | |
| CD45RA CD4 | 3.5 | Coronery artery | 6.7 |
| lymphocyte act | | SMC rest | |
| CD45RO CD4 | 3.0 | Coronery artery | 4.3 |
| lymphocyte act | | SMC TNFalpha + | |
| | | IL-1beta | |
| CD8 lymphocyte act | 6.2 | Astrocytes rest | 35.4 |
| Secondary CD8 | 3.4 | Astrocytes | 24.3 |
| lymphocyte rest | | TNFalpha + | |
| | | IL-1beta | |
| Secondary CD8 | 0.0 | KU-812 (Basophil) | 0.5 |
| lynphocyte act | | rest | |
| CD4 lymphocyte | 0.0 | KU-812 (Basophil) | 2.3 |
| none | | PMA/ionomycin | |
| 2ry Th1/Th2/ | 0.0 | CCD1106 | 39.8 |
| Tr1_anti- | | (Keratinocytes) | |
| CD95 CH11 | | none | |
| LAK cells rest | 0.7 | CCD1106 | 32.5 |
| | | (Keratinocytes) | |
| | | TNFalpha + IL-1beta | |
| LAK cells IL-2 | 0.4 | Liver cirrhosis | 41.2 |
| LAK cells IL-2 + | 1.3 | NCI-H292 none | 49.3 |
| IL-12 | | | |
| LAK cells IL-2 + | 2.2 | NCI-H292 IL-4 | 45.1 |
| IFN gamma | | | |
| LAK cells IL-2 + | 2.0 | NCI-H292 IL-9 | 100.0 |
| IL-18 | | | |
| LAK cells | 1.7 | NCI-H292 IL-13 | 51.8 |
| PMA/ionomycin | | | |
| NK Cells IL-2 rest | 0.5 | NCI-H292 IFN gamma | 53.6 |
| Two Way MLR | 0.4 | HPAEC none | 8.0 |
| 3 day | | | |
| Two Way MLR | 0.8 | HPAEC TNF alpha + | 6.1 |
| 5 day | | IL-1beta | |
| Two Way MLR | 1.5 | Long fibroblast none | 13.3 |
| 7 day | | | |
| PBMC rest | 0.6 | Lung fibroblast | 7.1 |
| | | TNF alpha + | |
| | | IL-1beta | |
| PBMC PWM | 4.3 | Lung fibroblast IL-4 | 3.6 |
| PBMC PHA-L | 3.6 | Lung fibroblast IL-9 | 8.5 |
| Ramos (B cell) none | 14.8 | Lung fibroblast IL-13 | 3.3 |
| Ramos (B cell) | 15.7 | Lung fibroblast IFN | 10.5 |
| ionomycin | | gamma | |
| B lymphocytes | 6.3 | Dermal fibroblast | 5.1 |
| PMW | | CCD1070 rest | |
| B lymphocytes | 6.5 | Dermal fibroblast | 5.6 |
| CD40L and IL-4 | | CCD1070 TNF alpha | |
| EOL-1 dbcAMP | 0.0 | Dermal fibroblast | 7.7 |
| | | CCD1070 IL-1beta | |
| EOL-1 dbcAMP | 0.0 | Dermal fibroblast IFN | 5.5 |
| PMA/ionomycin | | gamma | |
| Dendritic cells none | 0.5 | Dermal fibroblast IL-4 | 3.7 |
| Dendritic cells LPS | 0.0 | Dermal Fibroblasts | 1.5 |
| | | rest | |
| Dendritic cells anti- | 41.8 | Neutrophils TNFa + | 0.0 |
| CD40 | | LPS | |
| Monocytes rest | 100.0 | Neutrophils rest | 0.0 |
| Monocytes LPS | 77.4 | Colon | 34.6 |
| Macrophages rest | 62.4 | Lung | 4.3 |
| Macrophages LPS | 15.9 | Thymus | 9.9 |
| HUVEC none | 20.0 | Kidney | 51.4 |
| HUVEC starved | 30.6 | | |

CNS_neurodegeneration_v1.0 Summary: Ag3643 Results from one experiment with the CG59987-01 gene are not included. The amp plot indicates that there were experimental difficulties with this run.

General_screening_panel_v1.4 Summary: Ag3643 Expression of the CG59987-01 gene is highest in a breast cancer cell line (CT=25.3). In addition, significant levels of expression are seen in clusters of cell lines derived from brain, gastric, colon, lung, and ovarian cancers. In addition, expression overall appears to be higher in samples derived from cancer cell lines than in normal tissues. Thus, expression of this gene could be used as a marker to detect the presence of cancer. This gene encodes a homolog of rhophilin, a rho GTPase that is involved in a signaling pathway that regulates cell adhesion, among other functions. Therefore, therapeutic modulation of the expression or function of this gene may be effective in the treatment of these cancers.

Among tissues with metabolic function, this gene is expressed at moderate to low levels in pituitary, adipose, adrenal gland, pancreas, thyroid, skeletal muscle, and adult and fetal heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also expressed at moderate to low levels in the CNS and may be a small molecule target for the treatment of neurologic diseases.

Panel 4.1D Summary: Ag3643 Expression of the CG59987-01 gene is highest in NCI-H292 cells stimulated by IL-9(CT=29.2). The gene is also expressed in a cluster of treated and untreated NCI-H292 mucoepidermoid cell line samples. The transcript is also expressed at lower but still significant levels in both small airway and bronchial epithelium treated with IL-1 beta and TNF-alpha. In comparison, expression in the normal lung is relatively low. The expression of the transcript in activated normal epithelium as well as a cell line that is often used as a model for airway epithelium (NCI-H292 cells) suggests that this transcript may be important in the proliferation or activation of airway epithelium. Therefore, therapuetics designed with the protein encoded by this transcript could be important in the treatment of diseases which include lung airway inflammation such as asthma and COPD.

DN. CG59971-01 and CG59971-02: Leucine Rich Repeat Protein

Expression of gene CG59971-01 and variant CG59971-02 was assessed using the primer-probe set Ag3639, described in Table DNA. Results of the RTQ-PCR runs are shown in Tables DNB and DNC.

TABLE DNA
Probe Name Ag3639

| | | | Start | SEQ ID |
|---|---|---|---|---|
| Primers | Sequences | Length | Position | NO: |
| Forward | 5′ttctgaacttcagctacaat-3′ | 22 | 510 | 755 |
| Probe | TET-5′-cttagacagctccctgcgcctcttgt-3′-TAMRA | 26 | 543 | 756 |
| Reverse | 5′-acttgattgtggcttaggttca-3′ | 22 | 584 | 757 |

TABLE DNB
General_screening_panel_v1.4

| | Rel. | | Rel. |
|---|---|---|---|
| | Exp. | | Exp. |
| | (%) | | (%) |
| | Ag3639, | | Ag3639, |
| | Run | | Run |
| Tissue Name | 218234144 | Tissue Name | 218234144 |
| Adipose | 3.2 | Renal ca. TK-10 | 24.0 |
| Melanoma* | 17.9 | Bladder | 11.8 |
| Hs688(A).T | | | |
| Melanoma* | 12.6 | Gastric ca. (liver met.) | 35.6 |
| Hs688(B).T | | NCI-N87 | |
| Melanoma* M14 | 64.6 | Gastric ca. KATO III | 35.8 |
| Melanoma* | 15.5 | Colon ca. SW-948 | 8.0 |
| LOXIMVI | | | |
| Melanoma* SK- | 25.2 | Colon ca. SW480 | 55.5 |
| MEL-5 | | | |
| Squamous cell | 10.5 | Colon ca.* (SW480 | 32.8 |
| carcinoma SCC-4 | | met) SW620 | |
| Testis Pool | 8.9 | Colon ca. HT29 | 13.7 |
| Prostate ca.* (bone | 19.3 | Colon ca. HCT-116 | 34.9 |
| met) PC-3 | | | |
| Prostate Pool | 3.2 | Colon ca. CaCo-2 | 15.4 |
| Placenta | 16.3 | Colon cancer tissue | 14.3 |
| Uterus Pool | 1.0 | Colon ca. SW1116 | 7.9 |
| Ovarian ca. | 19.9 | Colon ca. Colo-205 | 13.3 |
| OVCAR-3 | | | |
| Ovarian ca. | 30.1 | Colon ca. SW-48 | 8.8 |
| SK-OV-3 | | | |
| Ovarian ca. | 14.8 | Colon Pool | 8.2 |
| OVCAR-4 | | | |
| Ovarian ca. | 60.7 | Small Intestine Pool | 7.7 |
| OVCAR-5 | | | |
| Ovarian ca. | 15.1 | Stomach Pool | 3.9 |
| IGROV-1 | | | |
| Ovarian ca. | 9.5 | Bone Marrow Pool | 2.7 |
| OVCAR-8 | | | |
| Ovary | 12.6 | Fetal Heart | 7.2 |
| Breast ca. MCF-7 | 54.0 | Heart Pool | 3.7 |
| Breast ca. MDA- | 31.2 | Lymph Node Pool | 10.4 |
| MB-231 | | | |
| Breast ca. BT 549 | 27.2 | Fetal Skeletal Muscle | 4.0 |
| Breast ca. T47D | 100.0 | Skeletal Muscle Pool | 3.6 |
| Breast ca. MDA-N | 20.4 | Spleen Pool | 8.4 |
| Breast Pool | 8.9 | Thymus Pool | 15.3 |
| Trachea | 10.3 | CNS cancer | 37.9 |
| | | (glio/astro) | |
| | | U87-MG | |
| Lung | 1.1 | CNS cancer | 26.2 |
| | | (glio/astro) | |
| | | U-118-MG | |
| Fetal Lung | 21.9 | CNS cancer | 22.4 |
| | | (neuro; met) | |
| | | SK-N-AS | |
| Lung ca. NCI-N417 | 4.5 | CNS cancer | 15.7 |
| | | (astro) | |
| | | SF-539 | |
| Lung ca. LX-1 | 32.8 | CNS cancer (astro) | 46.7 |
| | | SNB-75 | |
| Lung ca. NCI-H146 | 7.7 | CNS cancer (glio) | 12.2 |
| | | SNB-19 | |
| Lung ca. SHP-77 | 33.2 | CNS cancer (glio) | 31.4 |
| | | SF-295 | |
| Lung ca. A549 | 30.83 | Brain (Amygdala) | 7.9 |
| | | Pool | |
| Lung ca. NCI-H526 | 8.7 | Brain (cerebellum) | 27.4 |
| Lung ca. NCI-H23 | 23.2 | Brain (fetal) | 24.3 |
| Lung ca. NCI-H460 | 18.0 | Brain (Hippocampus) | 6.8 |
| | | Pool | |
| Lung ca. HOP-62 | 9.2 | Cerebral Cortex Pool | 8.0 |
| Lung ca. NCI-H522 | 22.5 | Brain | 8.3 |
| | | (Substantia nigra) | |
| | | Pool | |
| Liver | 0.8 | Brain (Thalamus) Pool | 10.7 |
| Fetal Liver | 8.8 | Brain (whole) | 13.8 |
| Liver ca. HepG2 | 19.3 | Spinal Cord Pool | 7.6 |
| Kidney Pool | 12.9 | Adrenal Gland | 14.1 |
| Fetal Kidney | 8.4 | Pituitary gland Pool | 6.6 |
| Renal ca. 786-0 | 20.0 | Salivary Gland | 3.0 |
| Renal ca. A498 | 5.2 | Thyroid (female) | 4.9 |
| Renal ca. ACHN | 34.4 | Pancreatic ca. | 20.9 |
| | | CAPAN2 | |
| Renal ca. UO-31 | 15.5 | Pancreas Pool | 10.7 |

TABLE DNC
Panel 4.1D

| | Rel. | | Rel. |
|---|---|---|---|
| | Exp. | | Exp. |
| | (%) | | (%) |
| | Ag3639, | | Ag3639, |
| | Run | | Run |
| Tissue Name | 169975065 | Tissue Name | 169975065 |
| Secondary Th1 act | 51.8 | HUVEC IL-beta | 27.0 |
| Secondary Th2 act | 68.8 | HUVEC IFN gamma | 31.2 |
| Secondary Tr1 act | 74.7 | HUVEC TNF alpha + | 28.9 |
| | | IFN gamma | |
| Secondary Th1 rest | 26.4 | HUVEC TNF alpha + | 26.4 |
| | | IL4 | |
| Secondary Th2 rest | 41.5 | HUVEC IL-11 | 22.4 |
| Secondary Tr1 rest | 24.1 | Lung Microvascular | 55.5 |
| | | EC none | |
| Primary Th1 act | 50.3 | Lung Microvascular | 36.1 |
| | | EC TNFalpha + | |
| | | IL-1beta | |
| Primary Th2 act | 75.8 | Microvascular Dermal | 23.5 |
| | | EC none | |
| Primary Tr1 act | 66.4 | Microvascular Dermal | 33.0 |
| | | EC TNFalpha + | |
| | | IL-1beta | |
| Primary Th1 rest | 19.8 | Bronchial epithelium | 22.8 |
| | | TNFalpha + IL1beta | |
| Primary Th2 rest | 33.9 | Small airway | 23.2 |
| | | epithelium none | |
| Primary Tr1 rest | 64.2 | Small airway | 37.1 |
| | | epithelium | |
| | | TNFalpha + IL-beta | |
| CD45RA CD4 | 35.4 | Coronery artery | 21.2 |
| lymphocyte act | | SMC rest | |
| CD45RO CD4 | 59.5 | Coronery artery | 13.4 |
| lymphocyte act | | SMC TNFalpha + | |
| | | IL-1beta | |
| CD8 lymphocyte act | 64.6 | Astrocytes rest | 23.3 |
| Secondary CD8 | 36.1 | Astrocytes | 25.2 |
| lymphocyte rest | | TNFalpha + | |
| | | IL-1beta | |
| Secondary CD8 | 45.1 | KU-812 (Basophil) | 22.4 |
| lynphocyte act | | rest | |
| CD4 lymphocyte | 20.0 | KU-812 (Basophil) | 32.8 |
| none | | PMA/ionomycin | |
| 2ry Th1/Th2/ | 44.1 | CCD1106 | 61.6 |
| Tr1_anti- | | (Keratinocytes) | |
| CD95 CH11 | | none | |
| LAK cells rest | 53.2 | CCD1106 | 49.0 |
| | | (Keratinocytes) | |
| | | TNFalpha + IL-1beta | |
| LAK cells IL-2 | 57.4 | Liver cirrhosis | 8.1 |
| LAK cells IL-2 + | 54.0 | NCI-H292 none | 46.7 |
| IL-12 | | | |
| LAK cells IL-2 + | 59.0 | NCI-H292 IL-4 | 45.1 |
| IFN gamma | | | |
| LAK cells IL-2 + | 61.6 | NCI-H292 IL-9 | 58.6 |
| IL-18 | | | |
| LAK cells | 39.0 | NCI-H292 IL-13 | 35.6 |
| PMA/ionomycin | | | |
| NK Cells IL-2 rest | 60.7 | NCI-H292 IFN gamma | 28.9 |
| Two Way MLR | 55.1 | HPAEC none | 18.3 |
| 3 day | | | |
| Two Way MLR | 36.6 | HPAEC TNF alpha + | 44.8 |
| 5 day | | IL-1beta | |
| Two Way MLR | 36.6 | Lung fibroblast none | 28.3 |
| 7 day | | | |
| PBMC rest | 22.2 | Lung fibroblast | 20.2 |
| | | TNF alpha + | |
| | | IL-1beta | |
| PBMC PWM | 62.4 | Lung fibroblast IL-4 | 29.1 |
| PBMC PHA-L | 42.9 | Lung fibroblast IL-9 | 50.7 |
| Ramos (B cell) none | 43.5 | Lung fibroblast IL-13 | 40.3 |
| Ramos (B cell) | 46.3 | Lung fibroblast IFN | 37.4 |
| ionomycin | | gamma | |
| B lymphocytes | 40.9 | Dermal fibroblast | 60.3 |
| PMW | | CCD1070 rest | |
| B lymphocytes | 78.5 | Dermal fibroblast | 92.7 |
| CD40L and IL-4 | | CCD1070 TNF alpha | |
| EOL-1 dbcAMP | 34.2 | Dermal fibroblast | 22.7 |
| | | CCD1070 IL-1beta | |
| EOL-1 dbcAMP | 62.0 | Dermal fibroblast IFN | 24.0 |
| PMA/ionomycin | | gamma | |
| Dendritic cells none | 65.1 | Dermal fibroblast IL-4 | 35.6 |
| Dendritic cells LPS | 25.3 | Dermal Fibroblasts | 21.8 |
| | | rest | |
| Dendritic cells anti- | 41.8 | Neutrophils TNFa + | 1.7 |
| CD40 | | LPS | |
| Monocytes rest | 100.0 | Neutrophils rest | 18.3 |
| Monocytes LPS | 77.4 | Colon | 11.2 |
| Macrophages rest | 62.4 | Lung | 17.7 |
| Macrophages LPS | 15.9 | Thymus | 81.8 |
| HUVEC none | 20.0 | Kidney | 18.6 |
| HUVEC starved | 30.6 | | |

CNS_neurodegeneration_v1.0 Summary: Ag3639 Results from one experiment with the CG59971-01 gene are not included. The amp plot indicates that there were experimental difficulties with this run.

General_screening_panel_v1.4 Summary: Ag3639 Expression of the CG59971-02 gene is ubiquitous in this panel, with highest expression in a breast cancer cell line (CT=26.6). Overall, expression of this gene appears to be higher in samples derived from cancer cell lines than in normal tissues. This widespread expression suggests that this gene product is involved in cell growth and prolideration. Thus, expression of this gene could be used as a marker to detect the presence of cancer. Furthermore, therapeutic modulation of the expression or function of this gene may be useful in the treatment of cancer.

In addition, this gene is expressed at much higher levels in fetal lung and liver (CTs=29-30) when compared to expression in the adult counterpart (CTs=33). Thus, expression of this gene may be used to differentiate between the fetal and adult sources of these tissue.

Among tissues with metabolic function, this gene is expressed at moderate to low levels in pituitary, adipose, adrenal gland, pancreas, thyroid, and adult and fetal skeletal muscle, heart, and liver. This widespread expression among these tissues suggests that this gene product may play a role in normal neuroendocrine and metabolic and that disregulated expression of this gene may contribute to neuroendocrine disorders or metabolic diseases, such as obesity and diabetes.

This gene is also highly expressed in the brain, with highest expression in the cerebellum (CT=28.5), with moderate expression in other CNS regions as well including, amygdala, hippocampus, cerebral cortex, substantia nigra and thalamus. This gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and may play significant but distinct roles in neural development and in the adult nervous system of humans (Ref. 1). In Drosophilia, the LRR region of axon guidance proteins has been shown to be critical for their function (especially in axon this gene shows high expression in the brain, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

### REFERENCES

1. Battye R., Stevens A., Perry R. L., Jacobs J. R. (2001) Repellent signaling by Slit requires the leucine-rich repeats. J. Neurosci. 21: 4290-4298.

Panel 4.1D Summary: Ag3639 The CG59971-01 gene is expressed at high to moderate levels in a wide range of cell types of significance in the immune response in health and disease. Highest expression of the gene is seen in resting monocytes (CT=28.6). Significant levels of expression are also seen in members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### Example D

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV5a has one SNP variant, whose variant positions for its nucleotide and amino acid sequences is numbered according to SEQ ID NOs:13 and 14, respectively. The nucleotide sequence of the NOV5a variant differs as shown in Table SNP1.

TABLE SNP1
NOV5a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13376274 | 143 | A | T | 47 | Gln | Leu |

NOV9a has eight SNP variants, whose variant positions for its nucleotide and amino acid sequences is numbered according to SEQ ID NOs:21 and 22, respectively. The nucleotide sequence of the NOV9a variant differs as shown in Table SNP2.

TABLE SNP2
NOV9a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13376043 | 230 | G | T | 72 | Glu | End |
| 13376044 | 341 | G | A | 109 | Gly | Arg |
| 13376045 | 441 | A | C | 142 | Gln | Pro |
| 13376046 | 532 | C | T | 172 | His | His |
| 13376050 | 1680 | T | C | 555 | Leu | Ser |
| 13376O49 | 1762 | G | T | 582 | Leu | Phe |
| 13376048 | 1818 | C | T | 601 | Ser | Leu |
| 13376047 | 1900 | A | G | 628 | Thr | Thr |

NOV14a has five SNP variants, whose variant positions for its nucleotide and amino acid sequences is numbered according to SEQ ID NOs:43 and 44, respectively. The nucleotide sequence of the NOV14a variant differs as shown in Table SNP3.

TABLE SNP3
NOV14a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13376438 | 1307 | T | C | 431 | Val | Ala |
| 13376437 | 1571 | A | G | 519 | His | Arg |
| 13376436 | 1625 | T | C | 537 | Val | Ala |
| 13376435 | 1646 | T | C | 544 | Val | Ala |
| 13376434 | 1667 | T | C | 551 | Ile | Thr |

NOV15a has twelve SNP variants, whose variant positions for its nucleotide and amino acid sequences is numbered according to SEQ ID NOs:53 and 54, respectively. The nucleotide sequence of the NOV14a variant differs as shown in Table SNP4.

TABLE SNP4
NOV15a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13376083 | 154 | A | G | 45 | Pro | Pro |
| 13376082 | 194 | T | C | 59 | Ser | Pro |
| 13376081 | 253 | G | A | 78 | Arg | Arg |
| 13376080 | 280 | G | A | 87 | Gln | Gln |
| 13376079 | 327 | C | T | 103 | Ala | Val |
| 13376078 | 338 | C | T | 107 | Pro | Ser |
| 13376077 | 366 | T | C | 116 | Ile | Thr |
| 13376076 | 502 | A | G | 161 | Lys | Lys |
| 13376069 | 1069 | A | G | 350 | Pro | Pro |
| 13376072 | 1137 | A | G | 373 | Glu | Gly |
| 13376071 | 1264 | T | C | 415 | Leu | Leu |
| 13376070 | 1367 | T | C | 450 | Ser | Pro |

NOV17a has four SNP variants, whose variant positions for its nucleotide and amino acid sequences is numbered according to SEQ ID NOs:61 and 62, respectively. The nucleotide sequence of the NOV17a variant differs as shown in Table SNP5.

TABLE SNP5
NOV17a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377433 | 175 | T | C | 55 | His | His |
| 13377432 | 223 | C | G | 71 | Pro | Pro |
| 13377431 | 538 | G | A | 176 | Thr | Thr |
| 13377430 | 680 | T | C | 224 | Phe | Leu |

NOV19a has one SNP variant, whose variant positions for its nucleotide and amino acid sequences is numbered according to SEQ ID NOs:71 and 72, respectively. The nucleotide sequence of the NOV19a variant differs as shown in Table SNP6.

TABLE SNP6
NOV19a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377434 | 1777 | T | A | 586 | Thr | Thr |

NOV21a has one SNP variant, whose variant positions for its nucleotide and amino acid sequences is numbered according to SEQ ID NOs:75 and 76, respectively. The nucleotide sequence of the NOV21a variant differs as shown in Table SNP7.

TABLE SNP7
NOV21a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377435 | 7503 | T | A | 2482 | Ala | Ala |

NOV38a has two SNP variants, whose variant positions for its nucleotide and amino acid sequences is numbered according to SEQ ID NOs:123 and 124, respectively. The nucleotide sequence of the NOV38a variant differs as shown in Table SNP8.

TABLE SNP8

NOV38a variants.

| | |
|---|---|
| Nucleotides | Amino Acids |

| | | | | | | |
|---|---|---|---|---|---|---|
| Variant | Position | Initial | Modified | Position | Initial | Modified |

| | | | | | | |
|---|---|---|---|---|---|---|
| 13377439 | 801 | G | A | 232 | Ser | Ser |
| 13377441 | 1595 | C | G | 497 | Pro | Arg |

NOV39a has three SNP variants, whose variant positions for its nucleotide and amino acid sequences is numbered according to SEQ ID NOs:125 and 126, respectively. The nucleotide sequence of the NOV39a variant differs as shown in Table SNP9.

TABLE SNP9
NOV39a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13375670 | 183 | C | G | 57 | His | Gln |
| 13375669 | 187 | C | T | 59 | Leu | Phe |
| 13377389 | 1385 | A | G | 458 | His | Arg |

NOV46a has four SNP variants, whose variant positions for its nucleotide and amino acid sequences is numbered according to SEQ ID NOs:143 and 144, respectively. The nucleotide sequence of the NOV46a variant differs as shown in Table SNP10.

TABLE SNP10
NOV46a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377442 | 177 | T | C | 27 | Val | Ala |
| 13377443 | 590 | A | G | 165 | Thr | Ala |
| 13377444 | 799 | A | G | 234 | Gln | Gln |
| 13377445 | 977 | T | C | 294 | Tyr | His |

NOV49a has two SNP variants, whose variant positions for its nucleotide and amino acid sequences is numbered according to SEQ ID NOs:151 and 152, respectively. The nucleotide sequence of the NOV49a variant differs as shown in Table SNP11.

TABLE SNP11
NOV49a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377450 | 119 | A | G | 7 | Arg | Gly |
| 13377448 | 1556 | G | A | 486 | Ala | Thr |

NOV50a has one SNP variant, whose variant positions for its nucleotide and amino acid sequences is numbered according to SEQ ID NOs:153 and 154, respectively. The nucleotide sequence of the NOV50a variant differs as shown in Table SNP 12.

TABLE SNP12
NOV50a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377451 | 2371 | G | A | 791 | Ala | Thr |

NOV51a has five SNP variants, whose variant positions for its nucleotide and amino acid sequences is numbered according to SEQ ID NOs:155 and 156, respectively. The nucleotide sequence of the NOV51a variant differs as shown in Table SNP13.

TABLE SNP13
NOV51a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13374492 | 765 | G | A | 243 | Ala | Thr |
| 13374491 | 924 | T | C | 296 | Phe | Leu |
| 13377453 | 1028 | C | T | 330 | Pro | Pro |
| 13377454 | 1052 | A | C | 338 | Ala | Ala |
| 13377455 | 1205 | C | T | 389 | His | His |

NOV52a has one SNP variant, whose variant positions for its nucleotide and amino acid sequences is numbered according to SEQ ID NOs:157 and 158, respectively. The nucleotide sequence of the NOV52a variant differs as shown in Table SNP14.

TABLE SNP14
NOV52a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377458 | 221 | C | T | 37 | Arg | Trp |

NOV55a has one SNP variant, whose variant positions for its nucleotide and amino acid sequences is numbered according to SEQ ID NOs:163 and 164, respectively. The nucleotide sequence of the NOV55a variant differs as shown in Table SNP15.

TABLE SNP15
NOV55a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377462 | 514 | C | T | 165 | Arg | Trp |
| 13377461 | 993 | T | C | 324 | Ser | Ser |

NOV60a has one SNP variant, whose variant positions for its nucleotide and amino acid sequences is numbered according to SEQ ID NOs:183 and 184, respectively. The nucleotide sequence of the NOV55a variant differs as shown in Table SNP16.

TABLE SNP16
NOV60a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377463 | 453 | T | C | 111 | Gly | Gly |

NOV65a has one SNP variant, whose variant positions for its nucleotide and amino acid sequences is numbered according to SEQ ID NOs:195 and 196, respectively. The nucleotide sequence of the NOV65a variant differs as shown in Table SNP17.

TABLE SNP17
NOV65a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377464 | 75 | C | A | 25 | Gln | Lys |

NOV68a has one SNP variant, whose variant positions for its nucleotide and amino acid sequences is numbered according to SEQ ID NOs:201 and 202, respectively. The nucleotide sequence of the NOV68a variant differs as shown in Table SNP 18.

TABLE SNP18
NOV68a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377465 | 438 | C | G | 145 | Gly | Gly |

NOV72a has one SNP variant, whose variant positions for its nucleotide and amino acid sequences is numbered according to SEQ ID NOs:209 and 210, respectively. The nucleotide sequence of the NOV72a variant differs as shown in Table SNP19.

TABLE SNP19
NOV72a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377466 | 839 | C | T | 271 | Pro | Ser |

NOV80a has four SNP variants, whose variant positions for its nucleotide and amino acid sequences is numbered according to SEQ ID NOs:225 and 226, respectively. The nucleotide sequence of the NOV80a variant differs as shown in Table SNP20.

TABLE SNP20
NOV80a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377471 | 166 | G | T | 46 | Ala | Ser |
| 13377470 | 482 | C | T | 151 | Ala | Val |
| 13377469 | 685 | A | G | 219 | Thr | Ala |
| 13377468 | 1410 | G | C | 460 | Glu | Asp |

NOV81a has four SNP variants, whose variant positions for its nucleotide and amino acid sequences is numbered according to SEQ ID NOs:229 and 230, respectively. The nucleotide sequence of the NOV81a variant differs as shown in Table SNP21.

TABLE SNP21
NOV81a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377472 | 285 | C | T | 91 | His | Tyr |
| 13377473 | 553 | A | G | 180 | His | Arg |
| 13377474 | 554 | C | T | 180 | His | His |
| 13377475 | 2581 | A | G | 856 | Gln | Arg |

NOV89a has two SNP variants, whose variant positions for its nucleotide and amino acid sequences is numbered according to SEQ ID NOs:249 and 250, respectively. The nucleotide sequence of the NOV89a variant differs as shown in Table SNP22.

TABLE SNP22
NOV89a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377477 | 425 | A | G | 119 | Met | Val |
| 13377478 | 1162 | C | A | 364 | Val | Val |

NOV94a has one SNP variants, whose variant positions for its nucleotide and amino acid sequences is numbered according to SEQ ID NOs:269 and 270, respectively. The nucleotide sequence of the NOV94a variant differs as shown in Table SNP23.

TABLE SNP23
NOV94a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377479 | 1005 | T | C | 303 | Asp | Asp |

NOV96a has two SNP variants, whose variant positions for its nucleotide and amino acid sequences is numbered according to SEQ ID NOs:273 and 274, respectively. The nucleotide sequence of the NOV96a variant differs as shown in Table SNP24.

TABLE SNP24
NOV96a variants.

| | |
|---|---|
| Nucleotides | Amino Acids |

| | | | | | | |
|---|---|---|---|---|---|---|
| | Posi- | | Modi- | Posi- | | Modi- |
| Variant | tion | Initial | fied | tion | Initial | fied |

| | | | | | | |
|---|---|---|---|---|---|---|
| 13377480 | 150 | A | G | 45 | Lys | Arg |
| 13377482 | 2221 | A | G | 735 | Ser | Ser |

NOV99a has one SNP variant, whose variant positions for its nucleotide and amino acid sequences is numbered according to SEQ ID NOs:283 and 284, respectively. The nucleotide sequence of the NOV99a variant differs as shown in Table SNP25.

TABLE SNP25

NOV99a variants.

| | |
|---|---|
| Nucleotides | Amino Acids |

| | | | | | | |
|---|---|---|---|---|---|---|
| Posi- | | Modi- | Posi- | | Modi- | |
| Variant | tion | Initial | fied | tion | Initial | fied |

| | | | | | | |
|---|---|---|---|---|---|---|
| 13377485 | 274 | T | C | 78 | Thr | Thr |

NOV105a has three SNP variants, whose variant positions for its nucleotide and amino acid sequences is numbered according to SEQ ID NOs:299 and 300, respectively. The nucleotide sequence of the NOV105a variant differs as shown in Table SNP26.

TABLE SNP26

NOV105a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377488 | 453 | C | T | 145 | Ile | Ile |
| 13377487 | 828 | T | G | 270 | Thr | Thr |
| 13377486 | 924 | A | G | 302 | Thr | Thr |

NOV113a has three SNP variants, whose variant positions for its nucleotide and amino acid sequences is numbered according to SEQ ID NOs:315 and 316, respectively. The nucleotide sequence of the NOV113a variant differs as shown in Table SNP27.

TABLE SNP27

NOV113a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377490 | 340 | G | A | 100 | Ala | Thr |
| 13377491 | 659 | C | T | 206 | Pro | Leu |
| 13377492 | 726 | C | T | 228 | Arg | Arg |
| 13377493 | 915 | T | C | 291 | Ala | Ala |
| 13377494 | 1058 | T | C | 339 | Ile | Thr |
| 13377495 | 1088 | T | C | 349 | Leu | Pro |

NOV114a has two SNP variants, whose variant positions for its nucleotide and amino acid sequences is numbered according to SEQ ID NOs:319 and 320, respectively. The nucleotide sequence of the NOV114a variant differs as shown in Table SNP28.

TABLE SNP28

NOV114a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13375633 | 185 | T | C | 54 | Val | Ala |
| 13375632 | 689 | A | G | 222 | Gln Arg | |

NOV116a has two SNP variants, whose variant positions for its nucleotide and amino acid sequences is numbered according to SEQ ID NOs:325 and 326, respectively. The nucleotide sequence of the NOV116a variant differs as shown in Table SNP29.

TABLE SNP29

NOV116a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13374815 | 203 | A | G | 63 | Thr | Ala |
| 13374814 | 384 | T | C | 123 | Leu | Pro |

NOV117a has three SNP variants, whose variant positions for its nucleotide and amino acid sequences is numbered according to SEQ ID NOs:329 and 330, respectively. The nucleotide sequence of the NOV117a variant differs as shown in Table SNP30.

TABLE SNP30

NOV117a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377507 | 453 | A | G | 149 | Pro | Pro |
| 13377506 | 755 | A | T | 250 | Glu | Val |
| 13377505 | 1128 | G | A | 374 | Lys | Lys |

NOV124a has six SNP variants, whose variant positions for its nucleotide and amino acid sequences is numbered according to SEQ ID NOs:343 and 344, respectively. The nucleotide sequence of the NOV124a variant differs as shown in Table SNP31.

TABLE SNIP31
NOV124a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377511 | 186 | C | T | 39 | Ser | Ser |
| 13377512 | 372 | C | T | 101 | Gly | Gly |
| 13377513 | 970 | G | T | 301 | Asp | Tyr |
| 13377514 | 1051 | G | A | 328 | Val | Met |
| 13377515 | 1266 | C | T | 399 | Ile | Ile |
| 13377516 | 1304 | A | G | 412 | Asp | Gly |

NOV126a has two SNP variants, whose variant positions for its nucleotide and amino acid sequences is numbered according to SEQ ID NOs:349 and 350, respectively. The nucleotide sequence of the NOV126a variant differs as shown in Table SNP32.

TABLE SNP32

NOV126a variants.

| | |
|---|---|
| Nucleotides | Amino Acids |

| | Posi- | | Modi- | Posi- | | Modi- |
|---|---|---|---|---|---|---|
| Variant | tion | Initial | fied | tion | Initial | fied |
| 13377517 | 747 | T | C | 234 | Cys | Cys |

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising an amino acid sequence selected from the group consisting of:
a) a mature form of the amino acid sequence selected from the group consisting of SEQ ID NO: 2n, wherein n is an integer between 1 and 178;
b) a variant of a mature form of the amino acid sequence selected from the group consisting of SEQ ID NO: 2n, wherein n is an integer between 1 and 178, wherein any amino acid in the mature form is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 178;
d) a variant of the amino acid sequence selected from the group consisting of SEQ ID NO: 2n, wherein n is an integer between 1 and 178 wherein any amino acid specified in the chosen sequence is changed to a different amino acid, provided that no more than 15% of the amino acid residues in the sequence are so changed; and
e) a fragment of any of a) through d).

2. The polypeptide of claim 1 that is a naturally occurring allelic variant of the sequence selected from the group consisting of SEQ ID NO: 2n, wherein n is an integer between 1 and 178.

3. The polypeptide of claim 2, wherein said allelic variant comprises an amino acid sequence that is the translation of a nucleic acid sequence differing by a single nucleotide from a nucleic acid sequence selected from the group consisting of SEQ ID NO: 2n, wherein n is an integer between 1 and 178.

4. The polypeptide of claim 1 that is a variant polypeptide described therein, wherein any amino acid specified in the chosen sequence is changed to provide a conservative substitution.

5. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

6. A method for determining the presence of or predisposition to a disease associated with altered levels of the polypeptide of claim 1 in a first mammalian subject, the method comprising:
a) measuring the level of expression of the polypeptide in a sample from the first mammalian subject; and
b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

7. A method of identifying an agent that binds to the polypeptide of claim 1, the method comprising:
(a) introducing said polypeptide to said agent; and
(b) determining whether said agent binds to said polypeptide.

8. The method of claim 7 wherein the agent is a cellular receptor or a downstream effector.

9. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition devoid of the substance, the substance is identified as a potential therapeutic agent.

10. A method for screening for a modulator of activity or of latency or predisposition to a pathology associated with the polypeptide of claim 1, said method comprising:
a) administering a test compound to a test animal at increased risk for a pathology associated with the polypeptide of claim 1, wherein said test animal recombinantly expresses the polypeptide of claim 1;
b) measuring the activity of said polypeptide in said test animal after administering the compound of step (a); and
c) comparing the activity of said protein in said test animal with the activity of said polypeptide in a control animal not administered said polypeptide, wherein a change in the activity of said polypeptide in said test animal relative to said control animal indicates the test compound is a modulator of latency of, or predisposition to, a pathology associated with the polypeptide of claim 1.

11. The method of claim 10, wherein said test animal is a recombinant test animal that expresses a
test protein transgene or expresses said transgene under the control of a promoter at an increased level relative to a wild-type test animal, and wherein said promoter is not the native gene promoter of said transgene.

12. A method for modulating the activity of the polypeptide of claim 1, the method comprising introducing a cell sample expressing the polypeptide of said claim with a compound that binds to said polypeptide in an amount sufficient to modulate the activity of the polypeptide.

13. An isolated nucleic acid molecule comprising a nucleic acid sequence encoding a polypeptide comprising an amino acid sequence selected from the group consisting of:
a) a mature form of the amino acid sequence given SEQ ID NO: 2n, wherein n is an integer between 1 and 178;
b) a variant of a mature form of the amino acid sequence selected from the group consisting of SEQ ID NO: 2n, wherein n is an integer between 1 and 178 wherein any amino acid in the mature form of the chosen sequence is changed to a different amino acid, provided that no more than 15% of the amino acid residues in the sequence of the mature form are so changed;
c) the amino acid sequence selected from the group consisting of SEQ ID NO: 2n, wherein n is an integer between 1 and 178;
d) a variant of the amino acid sequence selected from the group consisting of SEQ ID NO: 2n, wherein n is an integer between 1 and 178, in which any amino acid specified in the chosen sequence is changed to a different amino acid, provided that no more than 15% of the amino acid residues in the sequence are so changed;
e) a nucleic acid fragment encoding at least a portion of a polypeptide comprising the amino acid sequence selected from the group consisting of SEQ ID NO: 2n, wherein n is an integer between 1 and 178 or any variant of said polypeptide wherein any amino acid of the chosen sequence is changed to a different amino acid, provided that no more than 10% of the amino acid residues in the sequence are so changed; and
f) the complement of any of said nucleic acid molecules.

14. The nucleic acid molecule of claim 13, wherein the nucleic acid molecule comprises the nucleotide sequence of a naturally occurring allelic nucleic acid variant.

15. The nucleic acid molecule of claim 13 that encodes a variant polypeptide, wherein the variant polypeptide has the polypeptide sequence of a naturally occurring polypeptide variant.

16. The nucleic acid molecule of claim 13, wherein the nucleic acid molecule differs by a single nucleotide from a nucleic acid sequence selected from the group consisting of SEQ ID NO: 2n−1, wherein n is an integer between 1 and 178.

17. The nucleic acid molecule of claim 13, wherein said nucleic acid molecule comprises a nucleotide sequence selected from the group consisting of
a) the nucleotide sequence selected from the group consisting of SEQ ID NO: 2n−1, wherein n is an integer between 1 and 178;
b) a nucleotide sequence wherein one or more nucleotides in the nucleotide sequence selected from the group consisting of SEQ ID NO: 2n−1, wherein n is an integer between 1 and 178 is changed from that selected from the group consisting of the chosen sequence to a different nucleotide provided that no more than 15% of the nucleotides are so changed;
c) a nucleic acid fragment of the sequence selected from the group consisting of SEQ ID NO: 2n−1, wherein n is an integer between 1 and 178; and
d) a nucleic acid fragment wherein one or more nucleotides in the nucleotide sequence selected from the group consisting of SEQ ID NO: 2n−1, wherein n is an integer between 1 and 178 is changed from that selected from the group consisting of the chosen sequence to a different nucleotide provided that no more than 15% of the nucleotides are so changed.

18. The nucleic acid molecule of claim 13, wherein said nucleic acid molecule hybridizes under stringent conditions to the nucleotide sequence selected from the group consisting of SEQ ID NO: 2n−1, wherein n is an integer between 1 and 178, or a complement of said nucleotide sequence.

19. The nucleic acid molecule of claim 13, wherein the sequence is changed such that no more than 15% of the nucleotides in the coding sequence differ from the nucleotide sequence selected from the group consisting of SEQ ID NO: 2n−1, wherein n is an integer between 1 and 178 or a fragment thereof.

20. A vector comprising the nucleic acid molecule of claim 19.

21. The vector of claim 20, further comprising a promoter operably linked to said nucleic acid molecule.

22. A cell comprising the vector of claim 20.

23. A method for determining the presence or amount of the nucleic acid molecule of claim 13 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

24. The method of claim 23 wherein presence or amount of the nucleic acid molecule is used as a marker for cell or tissue type.

25. The method of claim 24 wherein the cell or tissue type is cancerous.

26. A method for determining the presence of or predisposition to a disease associated with altered levels of the nucleic acid molecule of claim 13 in a first mammalian subject, the method comprising:
a) measuring the amount of the nucleic acid in a sample from the first mammalian subject; and
b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.
